(19) United States
(12) Patent Application Publication
(10) Pub. No.: US 2026/0272945 A1
(43) Pub. Date: Sep. 17, 2026

(54) FUSED PYRIMIDINE COMPOUNDS AS INHIBITORS OF MENIN

(71) Applicant: Biomea Fusion, Inc., Redwood City, CA (US)

(72) Inventors: Yeyu CAO, Foster City, CA (US); James T. PALMER, Warrandyte (AU); David SPERANDIO, San Carlos, CA (US); Thorsten A. KIRSCHBERG, San Carlos, CA (US); Thomas BUTLER, Redwood City, CA (US); Xiaodong WANG, Millbrae, CA (US); Solomon B. UNGASHE, Sunnyvale, CA (US); Nan-Horng LIN, Vernon Hills, IL (US); Ravindra B. UPASANI, San Jose, CA (US); Amna Trinity-Turjuman ADAM, Mountain View, CA (US); Neil Howard SQUIRES, San Francisco, CA (US); Thu PHAN, Fremont, CA (US); Yongli SU, Foster City, CA (US); Satish Goud PUPPALI, Sunnyvale, CA (US)

(21) Appl. No.: 19/489,148

(22) PCT Filed: May 31, 2024
(86) PCT No.: PCT/US2024/032133

§ 371 Date: Dec. 1, 2025

Related U.S. Application Data
(60) Provisional application No. 63/505,848, filed on Jun. 2, 2023.
(60) Provisional application No. 63/515,973, filed on Jul. 27, 2023.
(60) Provisional application No. 63/606,856, filed on Dec. 6, 2023.

(51) Int. Cl. A61K 31/541; A61K 31/519; A61K 31/5377; A61K 31/5386; A61P 35/00; A61P 35/02; C07D 471/04; C07D 487/04; C07D 519/00

(52) U.S. Cl. CPC A61K 31/541; A61K 31/519; A61K 31/5377; A61K 31/5386; A61P 35/00; A61P 35/02; C07D 471/04; C07D 487/04; C07D 519/00

(57) ABSTRACT

Disclosed herein are heterocyclic compounds that inhibit the binding of menin and MLL or MLL fusion proteins. Also described are specific covalent inhibitors of a menin or menin-MLL interaction. Also disclosed are pharmaceutical compositions that include the compounds described herein. Methods of using the menin or menin-MLL irreversible inhibitors are disclosed, alone or in combination with other therapeutic agents, for the treatment of autoimmune diseases or conditions, heteroimmune diseases or conditions, cancer, including lymphoma, leukemia and other diseases or conditions dependent on menin or menin-MLL interaction.

### CROSS-REFERENCE

This application claims the benefit of U.S. Provisional Application Nos. 63/505,848, filed Jun. 2, 2023, 63/515,973, filed Jul. 27, 2023, and 63/606,856, filed Dec. 6, 2023, the contents of which are hereby incorporated by reference in their entireties.

### FIELD

Described herein are compounds, methods of making such compounds, pharmaceutical compositions, and medicaments containing such compounds, and methods of using such compounds and compositions to inhibit the activity of menin.

### BACKGROUND

The Histone-lysine N-methyltransferase 2 (KMT2) family of proteins, which currently consists of at least 5 members, methylate lysine 4 on the histone H3 tails at important regulatory regions in the genome and thereby impart crucial functions through the modulation of chromatin structures and DNA accessibility (Morera, Lübbert, and Jung., Clin. Epigenetics 8, 57-(2016)). These enzymes are known to play an important role in the regulation of gene expression during early development and hematopoiesis (Rao & Dou, Nat.Rev. Cancer 15, 334-346 (2015)).

The human KMT2 family was initially named the mixed-lineage leukaemia (MLL) family, owing to the role of the first-found member in this disease, KMT2A which is still commonly referred to as MLL1 or MLL in routine clinical practice.

KMT2A (MLL1) is frequently found to be cytogenetically targeted in several types of leukemia (e.g., ALL and AML), and in those cases where balanced chromosomal translocations are found, these typically target KMT2A (MLL1) and one of over 80 translocation partner genes that have been described to date (Winters and Bernt, Front. Pediatr. 5, 4 (2017)). These chromosomal anomalies often result in the formation of fusion genes that encode fusion proteins which are believed to be causally related to the onset and/or progression of the disease. Inhibition of menin may be a promising strategy for treating MLL related diseases, including leukemia.

M-525 is a highly potent, irreversible small molecule inhibitor of the menin-MLL protein-protein interaction. It forms a covalent bond with Cys329 residue in menin. M-525 demonstrates high cellular specificity over non-MLL leukemia cells and is >30 times more potent that the corresponding reversible inhibitors. [see S. Xu et al. Angewandte Chemie International Ed. 57 (6), 1601-1605 (2017)].

### SUMMARY

Described herein are inhibitors of menin. Also described herein are specific heterocyclic inhibitors of menin. In some embodiments, the inhibitors of menin are irreversible inhibitors. In some embodiments, the inhibitors of menin are covalent inhibitors. In some embodiments, the inhibitors of menin are reversible inhibitors.

Described herein are inhibitors of menin and/or menin-MLL interaction. Also described herein are specific heterocyclic inhibitors of menin or menin-MLL or MLL fusion protein interaction. In some embodiments, the inhibitors are irreversible inhibitors. In some embodiments, the inhibitors are covalent inhibitors. In some embodiments, the inhibitors are reversible inhibitors.

Also, described herein are covalent inhibitors of menin. Also described herein are specific heterocyclic irreversible inhibitors of menin.

Also, described herein are irreversible inhibitors of menin and/or menin-MLL interaction. Also described herein are specific heterocyclic irreversible inhibitors of menin and/or menin-MLL or MLL fusion proteins interaction.

Also described herein are methods for synthesizing such covalent inhibitors, methods for using such covalent inhibitors in the treatment of diseases (including diseases wherein inhibition of menin provides therapeutic benefit to a patient having the disease). Further described are pharmaceutical compositions that include an inhibitor of menin. Further described are pharmaceutical compositions that include an inhibitor of menin and/or menin-MLL interaction. Further described are pharmaceutical compositions that include an inhibitor of menin. Specifically, described herein are compounds and methods of use thereof to inhibit interaction of menin with MLL oncoproteins (e.g., MLL1, MLL2, MLL-fusion oncoproteins).

Specifically described herein are irreversible inhibitors of menin and/or menin-MLL interaction that form a covalent bond with a cysteine residue on menin. Further described herein are irreversible inhibitors of menin and/or menin-MLL interaction that form a covalent bond with a Cys329 residue on menin. Also described are pharmaceutical formulations that include an irreversible inhibitor of menin.

Thus, in some embodiments, provided herein are compounds according to Formula (L-I) having the structure:

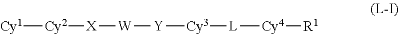

or a stereoisomer thereof; or a pharmaceutically acceptable salt thereof,
wherein:
Cy¹ is substituted or unsubstituted

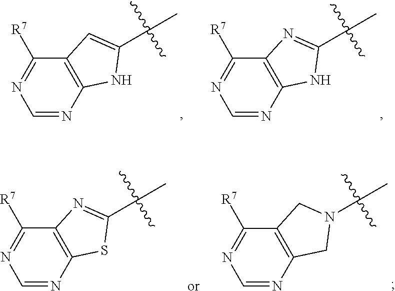

and the substitution on Cy¹ is C₁-C₄ alkyl, CN, or halo;
Cy² is substituted or unsubstituted cycloalkylene, substituted or unsubstituted heterocycloalkylene, substituted or unsubstituted arylene, or substituted or unsubstituted heteroarylene;
X is —NR^{3a}—, —C(R^{3b})₂—, or —O—;
W is —C(R^{3b})₂—, —C(O)—, —S(O)—, or —S(O)₂—;
Y is absent, —NR^{3a}—, —C(R^{3b})₂—, or —O—;
or X—W—Y is —N(H)—, —O—, or —S(O)₂—N(H)—C(R^{3b})₂—;
Cy³ is substituted or unsubstituted cycloalkylene, substituted or unsubstituted heterocycloalkylene, substituted or unsubstituted arylene, or substituted or unsubstituted heteroarylene;
L is a single bond, substituted or unsubstituted —N(H)—, —C(F)₂—O—, or substituted or unsubstituted C₁₋₄ alkylene; the substitution on alkylene is C₁-C₄ alkyl, CN, or halo; and the substitution on-N(H)— is C₁-C₄ alkyl;
i)R¹ is —B—C(R^{6a})═C(R^{6b})—C(O)—R^{6c}, —B—C(R^{6a})═C(R^{6b})—S(O)—R^{6c}, —B—C(R^{6a})—C(R^{6b})—S(O)₂—R^{6c}, —B—C(R^{6a})—C(R^{6b})—P(O)—R^{6a}R^{6b}; or —B—C(R^{6a})—C(R^{6b})—P(O)—OR^{6a}OR^{6b}; B is substituted or unsubstituted C₁₋₄ alkylene; R^{6c} is substituted or unsubstituted alkoxy, substituted or unsubstituted amino, or substituted or unsubstituted heterocycloalkyl, having 1-2 heteroatoms independently selected from nitrogen, oxygen, and sulfur; and Cy⁴ is absent, substituted or unsubstituted cycloalkylene, substituted or unsubstituted heterocycloalkylene, substituted or unsubstituted arylene, or substituted or unsubstituted heteroarylene; each R^{6a} and R^{6b} is independently H, CN, halo, or substituted or unsubstituted C₁₋₆ alkyl; and the substitution on C₁₋₆ alkyl is 1, 2, or 3 groups independently selected from halo, alkoxy, alkylamino, dialkylamino, or heterocycloalkyl;
ii)R¹ is —C(O)—C(R^{6a})—C(R^{6b})R^{6c}, —S(O)—C(R^{6a})═C(R^{6b})R^{6c}, —S(O)₂—C(R^{6a})═C(R^{6b})R^{6c}, —NR^{3c}—C(O)—C(R^{6a})—C(R^{6b})R^{6c}, —NR^{3c}—S(O)—C(R^{6a})—C(R^{6b})R^{6c}, or —NR^{3c}—S(O)₂—C(R^{6a})—C(R^{6b})R^{6c}, and Cy⁴ is absent;
iii)R¹ is —C(O)—C(R^{6a})—C(R^{6b})R^{6c}, —S(O)—C(R^{6a})═C(R^{6b})R^{6c}, —S(O)₂—C(R^{6a})═C(R^{6b})R^{6c}, —NR^{3c}—C(O)—C(R^{6a})—C(R^{6b})R^{6c}, —NR^{3c}—S(O)—C(R^{6a})—C(R^{6b})R^{6c}, or —NR^{3c}—S(O)₂—C(R^{6a})═C(R^{6b})R^{6c}; Cy⁴ is absent, substituted or unsubstituted cycloalkylene, substituted or unsubstituted heterocycloalkylene, substituted or unsubstituted arylene, or substituted or unsubstituted heteroarylene; and R^{6a} is alkyl, substituted with halo, alkoxy, alkylamino, dialkylamino, or heterocycloalkyl;
iv)R¹ is —C(O)—C(R^{6a})═C(R^{6b})R^{6c}, —S(O)—C(R^{6a})—C(R^{6b})R^{6c}, —S(O)₂—C(R^{6a})═C(R^{6b})R^{6c}, —NR^{3c}—C(O)—C(R^{6a})═C(R^{6b})R^{6c}, —NR^{3c}—S(O)—C(R^{6a})═C(R^{6b})R^{6c}, or —NR^{3c}—S(O)₂—C(R^{6a})═C(R^{6b})R^{6c}; Cy⁴ is substituted or unsubstituted azetidinylene, substituted or unsubstituted pyrrolidinylene, substituted or unsubstituted piperidinylene, or substituted or unsubstituted piperazinylene; and the substitution on each azetidinylene, pyrrolidinylene, piperidinylene, and piperazinylene is 1, 2, or 3 groups independently selected from halo, CN, hydroxy, substituted or unsubstituted C₁-C₄ alkyl, and substituted or unsubstituted C₁-C₄ alkoxy
or
v)R¹ is

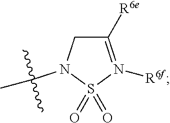

Cy⁴ is absent, substituted or unsubstituted azetidinylene, substituted or unsubstituted pyrrolidinylene, substituted or unsubstituted piperidinylene, or substituted or unsubstituted piperazinylene;
each R^{3a}, R^{3b}, and R^{3c} is independently H or substituted or unsubstituted C₁₋₄ alkyl; and the substitution on each azetidinylene, pyrrolidinylene, piperidinylene, and piperazinylene is 1, 2, or 3 groups independently selected from halo, CN, hydroxy, substituted or unsubstituted C₁-C₄ alkyl, and substituted or unsubstituted C₁-C₄ alkoxy
each R^{6a} and R^{6b} is independently H, CN, halo, or substituted or unsubstituted C₁₋₆ alkyl; or R^{6a} and R^{6b} are joined together to form a bond;
R^{6c} is substituted or unsubstituted alkoxy, substituted or unsubstituted amino, substituted or unsubstituted heterocycloalkyl, having 1-2 heteroatoms independently selected from nitrogen, oxygen, and sulfur; each R^{6e} and R^{6f} is independently H, CN, halo, or C₁₋₆ alkyl; and the substitution on heterocycloalkyl is 1, 2, or 3 groups independently selected from C₁-C₄ alkyl, hydroxy, CN, C₁-C₄ alkoxy, and halo; and each substitution on amino and alkoxy is independently C₁-C₄ alkyl; and
R⁷ is a 4-10 membered substituted or unsubstituted heterocycloalkyl ring having 1-2 heteroatoms independently selected from nitrogen, oxygen, and sulfur, substituted or unsubstituted phenyl, an 8-10 membered substituted or unsubstituted bicyclic aryl ring, and a 5-6 membered substituted or unsubstituted heteroaryl ring having 1-4 heteroatoms independently selected from nitrogen, oxygen, and sulfur; and the substitution on the phenyl, and the bicyclic aryl is 1, 2, or 3 groups independently selected from C₁-C₄ alkyl, hydroxy, CN, C₁-C₄ alkoxy; and wherein the substitution on each alkyl is 1, 2, or 3 groups independently selected from halo, CN, hydroxy, and C₁-C₄ alkoxy; the substitution on each alkoxy is 1, 2, or 3 groups independently selected from halo, CN, and substituted or unsubstituted C₁-C₄ alkyl; the substitution on each cycloalkyl is 1, 2, or 3 groups independently selected from halo, CN, hydroxy, substituted or unsubstituted C₁-C₄ alkyl, and substituted or unsubstituted C₁-C₄ alkoxy; the substitution on each heterocycloalkyl is 1, 2, or 3 groups independently selected from halo, CN, hydroxy, substituted or unsubstituted C₁-C₄ alkyl, and substituted or unsubstituted C₁-C₄ alkoxy; the substitution on each aryl is 1, 2, or 3 groups independently selected from halo, haloalkyl, amino, dialkylamino, amido, CN, hydroxy, substituted or unsubstituted C₁-C₄ alkyl, and substituted or unsubstituted C₁-C₄ alkoxy; the substitution on each heteroaryl is 1-3 groups independently selected from halo, haloalkyl, amino, dialkylamino, amido, CN, hydroxy, substituted or unsubstituted C₁-C₄ alkyl, substituted or unsubstituted C₁-C₄ alkoxy, and oxo;
provided the compound is according to formula LXIa, LXIb, LXII, LXIII, LXIV, LXV, or LXVI;
or the compound is any one of the compounds listed in Table 1G, 1H, 1J, and IK.

In another aspect, provided herein is a compound according to Formula (L-I) having the structure:

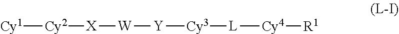

or a stereoisomer thereof; or a pharmaceutically acceptable salt thereof,
wherein:
Cy¹ is substituted or unsubstituted

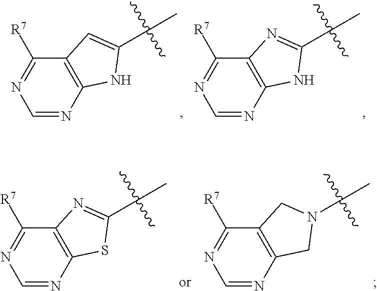

Cy² is substituted or unsubstituted

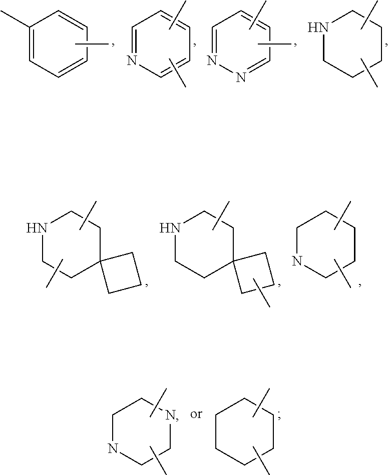

X is —NR^{3a}—, —C(R^{3b})₂—, or —O—;
W is —C(R^{3b})₂—, —C(O)—, —S(O)—, or —S(O)₂—;
Y is absent, —NR^{3a}—, —C(R^{3b})₂—, or —O—;
or X—W—Y is —N(H)—, —O—, or —S(O)₂—N(H)—C(R^{3b})₂—;
Cy³ is substituted or unsubstituted phenylene, pyridylene, pyrimidinylene, substituted or unsubstituted azetidinylene, substituted or unsubstituted pyrrolidinylene, substituted or unsubstituted piperidinylene, or substituted or unsubstituted piperazinylene;
L is a single bond, substituted or unsubstituted —N(H)—, —C(F)₂—O—, or substituted or unsubstituted C₁₋₄ alkylene;
Cy⁴ is absent, substituted or unsubstituted azetidinylene, substituted or unsubstituted pyrrolidinylene, substituted or unsubstituted piperidinylene, or substituted or unsubstituted piperazinylene;
R¹ is —C(O)—B—C(R^{6a})═C(R^{6b})—C(O)—R^{6c}, —S(O)—B—C(R^{6a})═C(R^{6b})—C(O)—R^{6c}, —S(O)₂—B—C(R^{6a})═C(R^{6b})—C(O)—R^{6c}, —B—C(R^{6a})—C(R^{6b})—C(O)—R^{6c}, —B—C(R^{6a})—C(R^{6b})—S(O)—R^{6c}, —B—C(R^{6a})—C(R^{6b})—S(O)₂—R^{6c}, —B—C(R^{6a})═C(R^{6b})—P(O)—R^{6a}R^{6b}, or —B—C(R^{6a})—C(R^{6b})—P(O)—OR^{6a}OR^{6b}, B is substituted or unsubstituted C₁₋₄ alkylene, substituted or unsubstituted cycloalkyl, or substituted or unsubstituted heterocycloalkyl;
each R^{3a}, R^{3b}, and R^{3c} is independently H or substituted or unsubstituted C₁₋₄ alkyl;
each R^{6a} and R^{6b} is independently H, CN, halo, or C₁₋₆ alkyl; or R^{6a} and R⁶ are joined together to form a bond; R^{6c} is substituted or unsubstituted alkoxy, substituted or unsubstituted amino, substituted or unsubstituted heterocycloalkyl, having 1-2 heteroatoms independently selected from nitrogen, oxygen, and sulfur;
R⁷ is H, an optionally substituted group selected from alkyl, a 4-10 membered heterocycloalkyl ring having 1-2 heteroatoms independently selected from nitrogen, oxygen, and sulfur, phenyl, an 8-10 membered bicyclic aryl ring, and a 5-6 membered heteroaryl ring having 1-4 heteroatoms independently selected from nitrogen, oxygen, and sulfur.

In another aspect, provided herein is a compound according to Formula (L-I) having the structure:

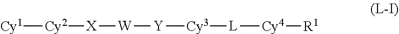

or a stereoisomer thereof; or a pharmaceutically acceptable salt thereof, wherein
Cy¹ is substituted or unsubstituted

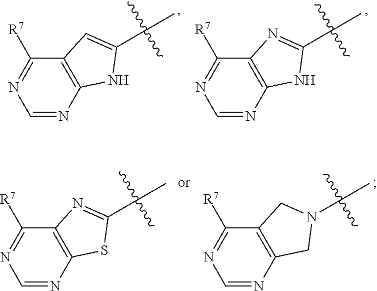

and the substitution on Cy¹ is C₁-C₄ alkyl, CN, or halo;
Cy² is substituted or unsubstituted

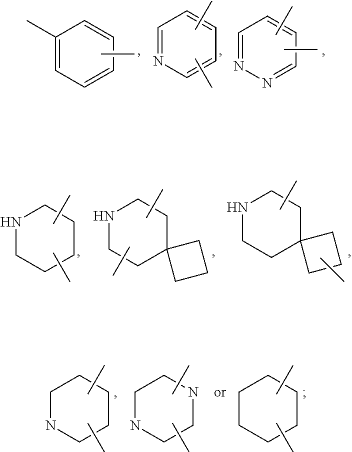

and the substitution on Cy² is selected from 1, 2, or 3 groups independently selected from C₁-C₄ alkyl, hydroxy, CN, C₁-C₄ alkoxy, and halo;
X is —NR^{3a}—, —C(R^{3b})₂—, or —O—;
W is —C(R^{3b})₂—, —C(O)—, —S(O)—, or —S(O)₂—;
Y is absent, —NR^{3a}—, —C(R^{3b})₂—, or —O—;
or X—W—Y is —N(H)—, —O—, or —S(O)₂—N(H)—C(R^{3b})₂—;
Cy³ is substituted or unsubstituted phenylene, pyridylene, pyrimidinylene, substituted or unsubstituted azetidinylene, substituted or unsubstituted pyrrolidinylene, substituted or unsubstituted piperidinylene, or substituted or unsubstituted piperazinylene; and the substitution on Cy³ is selected from 1, 2, or 3 groups independently selected from C₁-C₄ alkyl, hydroxy, CN, C₁-C₄ alkoxy, and halo;
L is a single bond, substituted or unsubstituted —N(H)—, —C(F)₂—O—, or substituted or unsubstituted C₁₋₄ alkylene; the substitution on alkylene is C₁-C₄ alkyl, CN, or halo; and the substitution on —N(H)— is C₁-C₄ alkyl;
i)R¹ is —B-C(R^{6a})═C(R^{6b})—C(O)—R^{6c}, —B—C(R^{6a})—C(R^{6b})—S(O)—R^{6c}, —B—C(R^{6a})—C(R^{6b})—S(O)₂—R^{6c}, —B—C(R^{6a})═C(R^{6b})—P(O)—R^{6a}R^{6b}; or —B—C(R^{6a})═C(R^{6b})—P(O)—OR^{6a}OR^{6b}; B is substituted or unsubstituted C₁₋₄ alkylene; R^{6c} is substituted or unsubstituted alkoxy, substituted or unsubstituted amino, or substituted or unsubstituted heterocycloalkyl, having 1-2 heteroatoms independently selected from nitrogen, oxygen, and sulfur; and Cy⁴ is absent, substituted or unsubstituted azetidinylene, substituted or unsubstituted pyrrolidinylene, substituted or unsubstituted piperidinylene, or substituted or unsubstituted piperazinylene; and the substitution on Cy⁴, and heterocycloalkyl is independently selected from 1, 2, or 3 groups independently selected from C₁-C₄ alkyl, hydroxy, CN, C₁-C₄ alkoxy, and halo; and the substitution on amino is C₁-C₄ alkyl or C₁-C₄ haloalkyl;
ii)R¹ is —C(O)—C(R^{6a})═C(R^{6b})R^{6c}, —S(O)—C(R^{6a})═C(R^{6b})R^{6c}, —S(O)₂—C(R^{6a})—C(R^{6b})R^{6c}, —NR^{3c}—C(O)—C(R^{6a})—C(R^{6b})R^{6c}, —NR^{3c}—S(O)—C(R^{6a})═C(R^{6b})R^{6c}, or —NR^{3c}—S(O)₂—C(R^{6a})—C(R^{6b})R^{6c}; and Cy⁴ is absent;
iii)R¹ is —C(O)—C(R^{6a})═C(R^{6b})R^{6c}, —S(O)—C(R^{6a})═C(R^{6b})R^{6c}, —S(O)₂—C(R^{6a})═C(R^{6b})R^{6c}, —NR^{3c}—C(O)—C(R^{6a})═C(R^{6b})R^{6c}, —NR^{3c}—S(O)—C(R^{6a})—C(R^{6b})R^{6c}, or —NR^{3c}—S(O)₂—C(R^{6a})═C(R^{6b})R^{6c}; Cy⁴ is substituted or unsubstituted azetidinylene, substituted or unsubstituted pyrrolidinylene, substituted or unsubstituted piperidinylene, or substituted or unsubstituted piperazinylene; and R^{6a} is alkyl, substituted with halo, alkoxy, alkylamino, dialkylamino, or heterocycloalkyl;
iv)R¹ is —C(O)—C(R^{6a})═C(R^{6b})R^{6c}, —S(O)—C(R^{6a})—C(R^{6b})R^{6c}, —S(O)₂—C(R^{6a})—C(R^{6b})R^{6c}, —NR^{3c}—C(O)—C(R^{6a})—C(R^{6b})R^{6c}, —NR^{3c}—S(O)—C(R^{6a})—C(R^{6b})R^{6c}, or —NR^{3c}—S(O)₂—C(R^{6a})═C(R^{6b})R^{6c}, Cy⁴ is substituted or unsubstituted azetidinylene, substituted or unsubstituted pyrrolidinylene, substituted or unsubstituted piperidinylene, or substituted or unsubstituted piperazinylene; and the compound is any one of compounds listed in 1e;
or
v)R¹ is

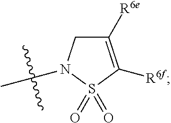

Cy⁴ is absent, substituted or unsubstituted azetidinylene, substituted or unsubstituted pyrrolidinylene, substituted or unsubstituted piperidinylene, or substituted or unsubstituted piperazinylene; each R^{3a}, R^{3b}, and R^{3c} is independently H or substituted or unsubstituted C₁₋₄ alkyl;
each R^{6a} and R^{6b} is independently H, CN, halo, or C₁₋₆ alkyl; or R^{6a} and R^{6b} are joined together to form a bond; R^{6c} is substituted or unsubstituted alkoxy, substituted or unsubstituted amino, substituted or unsubstituted heterocycloalkyl, having 1-2 heteroatoms independently selected from nitrogen, oxygen, and sulfur; each R^{6e} and Ref is independently H, CN, halo, or C₁₋₆ alkyl; and the substitution on heterocycloalkyl is independently selected from 1, 2, or 3 groups independently selected from C₁-C₄ alkyl, hydroxy, CN, C₁-C₄ alkoxy, and halo; and the substitution on amino and alkoxy is C₁-C₄ alkyl; and
R⁷ is a 4-10 membered heterocycloalkyl ring having 1-2 heteroatoms independently selected from nitrogen, oxygen, and sulfur, phenyl, an 8-10 membered bicyclic aryl ring, and a 5-6 membered heteroaryl ring having 1-4 heteroatoms independently selected from nitrogen, oxygen, and sulfur; and the substitution on heterocycloalkyl, phenyl, bicyclic aryl, and heteroaryl is independently selected from 1, 2, or 3 groups independently selected from C₁-C₄ alkyl, hydroxy, CN, C₁-C₄ alkoxy.

In another aspect, provided herein is a compound according to Formula (L-I); and wherein R³ª is H or substituted or unsubstituted C₁₋₄ alkyl; and each of R^{3b}, and R^{3c} is independently H, OH, substituted or unsubstituted C₁₋₄ alkyl, substituted or unsubstituted C₁₋₄ alkoxy.

In another aspect, provided herein is a compound according to Formula (L-I); and wherein Cy⁴ is substituted or unsubstituted heterocycloalkylene, and the heterocycloalkylene includes bicyclic, fused and bridged heterocycloalkyls. In one embodiment, Cy⁴ is a bridged heterocycloalkylene.

In another aspect, provided herein is a compound according to Formula (L-I); and wherein Cy⁴ is

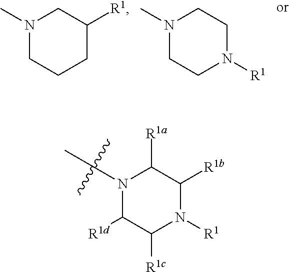

and wherein at least one pair of R^{1a} and R^{1c}, R^{1a} and R^{1d}, R^{1b} and R^{1c}, or R^{1b} and R^{1d} is joined together to form heterocycloalkyl, and the rest of R^{1a}, R^{1b}, R^{1c}, and R^{1d} are each H.

In another aspect, provided herein is a compound according to Formula (L-I) having the structure:

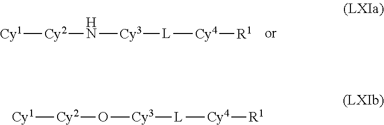

or a stereoisomer thereof; or a pharmaceutically acceptable salt thereof, wherein Cy¹, Cy², Cy³, Cy⁴, L, and R¹ are as described herein.

In another aspect, provided herein is a compound according to Formula (L-I) having the structure:

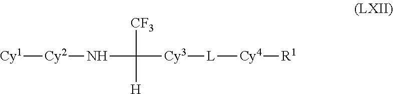

or a pharmaceutically acceptable salt thereof, wherein Cy¹, Cy², Cy³, Cy⁴, L, and R¹ are as described herein.

In another aspect, provided herein is a compound according to Formula (L-I) having the structure:

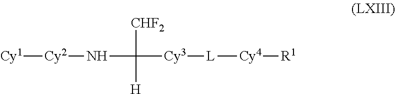

or a pharmaceutically acceptable salt thereof, wherein Cy¹, Cy², Cy³, Cy⁴, L, and R¹ are as described herein.

In another aspect, provided herein is a compound according to Formula (L-I) having the structure:

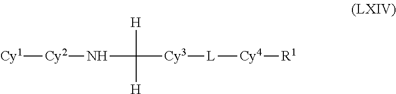

or a stereoisomer thereof; or a pharmaceutically acceptable salt thereof, wherein Cy¹, Cy², Cy³, Cy⁴, L, and R¹ are as described herein.

In another aspect, provided herein is a compound according to Formula (L-I) having the structure:

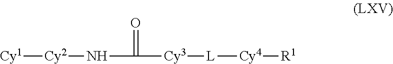

or a stereoisomer thereof; or a pharmaceutically acceptable salt thereof, wherein Cy¹, Cy², Cy³, Cy⁴, L, and R¹ are as described herein.

In another aspect, provided herein is a compound according to Formula (L-I) having the structure:

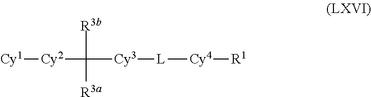

or a stereoisomer thereof; or a pharmaceutically acceptable salt thereof, wherein Cy¹, Cy², Cy³, Cy⁴, L, R¹, and R^{3a} are as described herein; and R^{3b} is OH, or substituted or unsubstituted C₁₋₄ alkoxy.

In one embodiment, R^{3b} is OH, or substituted or unsubstituted C₁₋₄ alkoxy; and R^{3a} is H, or substituted or unsubstituted C₁₋₄ alkyl. In one embodiment, R^{3b} is OH, or substituted or unsubstituted C₁₋₄ alkoxy; and R^{3a} is H, or C₁₋₄ alkyl, or halo C₁₋₄ alkyl. In one embodiment, R^{3b} is OH, or OMe; and R^{3a} is H, or Me, or CF₃.

In a particular embodiment, the compound is according to formula LXIa, LXIb, LXII, LXIII, LXIV, LXV, or LXVI. In another particular embodiment, the compound is according to formula LXV, and R^{6a} substituted alkyl. In a more particular embodiment, the compound is according to formula LXV, and R^{6a} methyl substituted with amino, alkylamino, dialkylamino, or heterocycloalkyl.

In certain particular embodiments, Cy¹ is

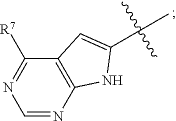

and R⁷ is as described herein.

In certain particular embodiments, R⁷ is

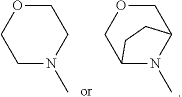

In certain particular embodiments, Cy² is phenylene.

In certain particular embodiments, Cy³ is pyridylene or pyrimidinylene. In certain particular embodiments, Cy³ is 2-pyridylene or 5-pyrimidinylene. In certain particular embodiments, Cy³ is piperidinylene. In certain particular embodiments, Cy³ is 4-piperidinylene. In certain particular embodiments, Cy³ is 1-piperidinylene.

In certain particular embodiments, L is a single bond. In certain particular embodiments, L is —CH₂—.

In some particular embodiments, Cy⁴ is absent.

In some particular embodiments, Cy⁴ is

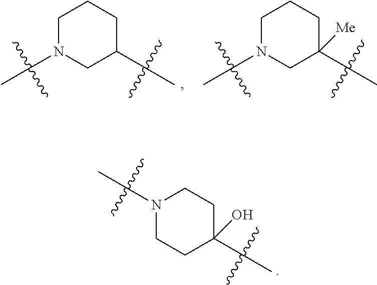 [SMILES: *C1CCCN(*)C1 ; *N1CCCC(*)(C)C1 ; *N1CCC(*)(O)CC1]

In some particular embodiments, Cy⁴ is

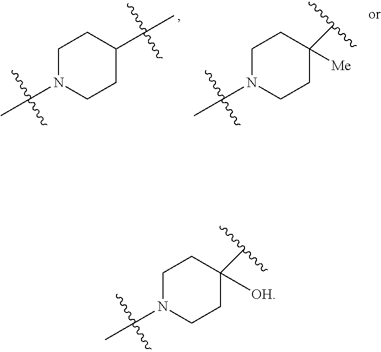 [SMILES: "or" ; "," ; *C1CCN(*)CC1 ; *N1CCC(*)(C)CC1 ; *N1CCC(*)(O)CC1]

In some particular embodiments, Cy⁴-R¹ is

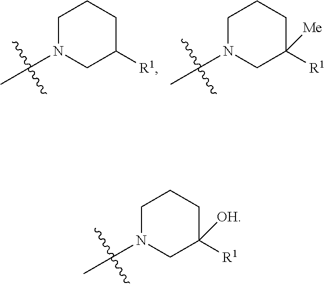

In some particular embodiments, Cy⁴ is

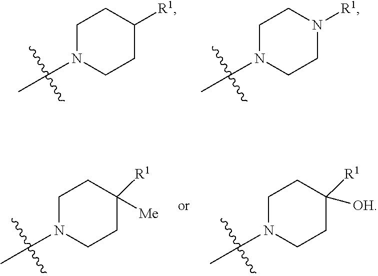

In some particular embodiments, Cy⁴ is

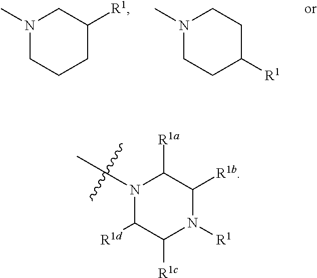

In some particular embodiments, —Cy³-L-Cy⁴-R¹ is

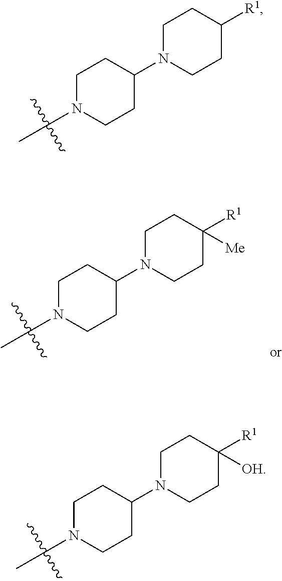

In some particular embodiments, —Cy³-L-Cy⁴-R¹ is

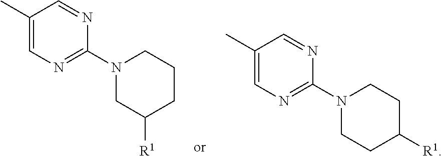

In some particular embodiments, —Cy³-L-Cy⁴-R¹ is

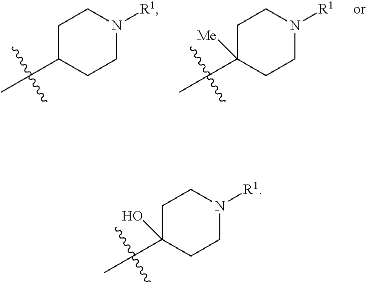

In certain embodiments, the compound is any one of compounds listed in Table 1E.

In certain embodiments, the compound is any one of compounds listed in Table 1C.

In certain embodiments, the compound is any one of compounds listed in Table 1D.

In certain embodiments, the compound is any one of compounds listed in Table 1F.

In certain embodiments, the compound is any one of compounds listed in Table 1G.

In certain embodiments, the compound is any one of compounds listed in Table 1H.

In certain embodiments, the compound is any one of compounds listed in Table 1J.

In certain embodiments, the compound is any one of compounds listed in Table 1K.

In certain embodiments, when R^{6a} and R^{6b} are joined together to form a bond, they form, or otherwise indicate, a triple bond between the adjacent atoms.

In some embodiments, provided herein are methods for preventing, treating or ameliorating in a mammal a disease or condition that is causally related to the aberrant activity of a menin and/or menin-MLL interaction in vivo, which comprises administering to the mammal an effective disease-treating or condition-treating amount of any of the compounds listed herein.

In some embodiments, the target site is a cavity in which the compound or the moiety binds to the MLL site on the menin. In some embodiments, the active site is MEN1 at the MLL binding site.

In some embodiments, the disease or condition is an autoimmune disease, a heteroimmune disease, a cancer, mastocytosis, osteoporosis or bone resorption disorder, or an inflammatory disease.

In some embodiments, the compounds provided herein may also serve as an anti-tumor agents through off-target activity by impacting other protein-protein interactions as well as kinases.

In some embodiments, provided herein are pharmaceutical compositions comprising a therapeutically effective amount of a compound of Formula (I) and a pharmaceutically acceptable excipient. In some embodiments, the pharmaceutical composition comprising the compound of Formula (I) is formulated for a route of administration selected from oral administration, parenteral administration, buccal administration, nasal administration, topical administration, or rectal administration. In some embodiments, provided herein are methods for treating an autoimmune disease or condition comprising administering to a patient in need a therapeutically effective amount of a compound of Formula (I). In some embodiments the autoimmune disease is selected from rheumatoid arthritis or lupus. In some embodiments, provided herein is a method for treating a heteroimmune disease or condition comprising administering to a patient in need a therapeutically effective amount of a compound of Formula (I). In some embodiments provided herein is a method for treating a cancer comprising administering to a patient in need a therapeutically effective amount of a compound of Formula (I). In some embodiments, the cancer is a myeloid line of blood cells. In some embodiments, the cancer is a lymphoid line of blood cell. In some embodiments, the cancer is a B-cell proliferative disorder. In some embodiments, the cancer is a lymphoid disorder.

In some embodiments the myeloid line of blood cells is acute myeloid leukemia. In some embodiments the lymphoid disorder is acute lymphoblastic leukemia. In some embodiments the B-cell proliferative disorder is diffuse large B cell lymphoma, follicular lymphoma, or chronic lymphocytic leukemia. In some embodiments the cancer (soft tissue) is glioblastoma or pancreatic cancer. In some embodiments the cancer is renal cell carcinoma.

In some embodiments, provided herein is a method for treating mastocytosis comprising administering to a patient in need a therapeutically effective amount of a compound of Formula (I).

In some embodiments, provided herein is a method for treating osteoporosis or bone resorption disorders comprising administering to a patient in need a therapeutically effective amount of a compound of Formula (I).

In some embodiments, provided herein is a method for treating an inflammatory disease or condition comprising administering to a patient in need a therapeutically effective amount of a compound of Formula (I).

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In some embodiments, provided herein are pharmaceutical compositions, which include a therapeutically effective amount of at least one of any of the compounds herein, or a pharmaceutically acceptable salt, pharmaceutically active metabolite, pharmaceutically acceptable prodrug, or pharmaceutically acceptable solvate. In certain embodiments, compositions provided herein further include a pharmaceutically acceptable diluent, excipient and/or binder.

Pharmaceutical compositions formulated for administration by an appropriate route and means containing effective concentrations of one or more of the compounds provided herein, or pharmaceutically effective derivatives thereof, that deliver amounts effective for the treatment, prevention, or amelioration of one or more symptoms of diseases, disorders or conditions that are modulated or otherwise affected by menin or menin-MLL activity, or in which menin or menin-MLL activity is implicated, are provided. The effective amounts and concentrations are effective for ameliorating any of the symptoms of any of the diseases, disorders or conditions disclosed herein.

In certain embodiments, provided herein is a pharmaceutical composition containing: i) a physiologically acceptable carrier, diluent, and/or excipient; and ii) one or more compounds provided herein.

In some embodiments, provided herein are methods for treating a patient by administering a compound provided herein. In some embodiments, provided herein is a method of inhibiting the activity of menin or menin-MLL, or of treating a disease, disorder, or condition, which would benefit from inhibition of menin or menin-MLL activity, in a patient, which includes administering to the patient a therapeutically effective amount of at least one of any of the compounds herein, or pharmaceutically acceptable salt, pharmaceutically active metabolite, pharmaceutically acceptable prodrug, or pharmaceutically acceptable solvate.

In some embodiments, provided herein is the use of a compound disclosed herein for inhibiting menin or menin-MLL activity or for the treatment of a disease, disorder, or condition, which would benefit from inhibition of menin or menin-MLL activity.

In some embodiments, compounds provided herein are administered to a human.

In some embodiments, compounds provided herein are orally administered.

In some embodiments, compounds provided herein are used for the formulation of a medicament for the inhibition of menin or menin-MLL activity. In some embodiments, compounds provided herein are used for the formulation of a medicament for the inhibition of menin or menin-MLL activity.

Articles of manufacture including packaging material, a compound or composition or pharmaceutically acceptable derivative thereof provided herein, which is effective for inhibiting the activity of menin or menin-MLL, within the packaging material, and a label that indicates that the compound or composition, or pharmaceutically acceptable salt, pharmaceutically active metabolite, pharmaceutically acceptable prodrug, or pharmaceutically acceptable solvate thereof, is used for inhibiting the activity of menin or menin-MLL, are provided.

In some embodiments, provided herein is a method for inhibiting menin or menin-MLL activity in a subject in need thereof by administering to the subject thereof a composition containing a therapeutically effective amount of at least one compound having the structure of Formula (I). In some embodiments, the subject in need is suffering from an autoimmune disease, e.g., inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitis, antiphospholipid antibody syndrome, aplastic anemia, autoimmune hepatitis, coeliac disease, Goodpasture's syndrome, idiopathic thrombocytopenia purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, or vulvodynia.

In some embodiments, the subject in need is suffering from a heteroimmune condition or disease, e.g., graft versus host disease, transplantation, transfusion, anaphylaxis, allergy, type I hypersensitivity, allergic conjunctivitis, allergic rhinitis, or atopic dermatitis.

In certain embodiments, the subject in need is suffering from an inflammatory disease, e.g., asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In some embodiments, the subject in need is suffering from a cancer. In some embodiments, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenström macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, where the subject is suffering from a cancer, an anti-cancer agent is administered to the subject in addition to one of the above-mentioned compounds.

In some embodiments, the subject in need is suffering from a thromboembolic disorder, e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In some embodiments, provided herein is a method for treating an autoimmune disease by administering to a subject in need thereof a composition containing a therapeutically effective amount of at least one compound having the structure of Formula (L-I), (L-II), and (I). In some embodiments, the autoimmune disease is arthritis. In some embodiments, the autoimmune disease is lupus. In some embodiments, the autoimmune disease is inflammatory bowel disease (including Crohn's disease and ulcerative colitis), rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitis, antiphospholipid antibody syndrome, aplastic anemia, autoimmune hepatitis, coeliac disease, Goodpasture's syndrome, idiopathic thrombocytopenia purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, or vulvodynia.

In some embodiments, provided herein is a method for treating a heteroimmune condition or disease by administering to a subject in need thereof a composition containing a therapeutically effective amount of at least one compound having the structure Formula (L-I), (L-II), and (I). In some embodiments, the heteroimmune condition or disease is graft versus host disease, transplantation, transfusion, anaphylaxis, allergy, type I hypersensitivity, allergic conjunctivitis, allergic rhinitis, or atopic dermatitis.

In some embodiments, provided herein is a method for treating an inflammatory disease by administering to a subject in need thereof a composition containing a therapeutically effective amount of at least one compound having the structure of Formula (L-I), (L-II), and (I). In some embodiments, the inflammatory disease is asthma, inflammatory bowel disease (including Crohn's disease and ulcerative colitis), appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In some embodiments, provided herein is a method for treating a cancer by administering to a subject in need thereof a composition containing a therapeutically effective amount of at least one compound having the structure of Formula (L-I), (L-II), and (I). In some embodiments, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenström macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, where the subject is suffering from a cancer, an anti-cancer agent is administered to the subject in addition to one of the above-mentioned compounds.

In some embodiments, provided herein is a method for treating a thromboembolic disorder by administering to a subject in need thereof a composition containing a therapeutically effective amount of at least one compound having the structure of Formula (L-I), (L-II), and (I). In some embodiments, the thromboembolic disorder is myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In some embodiments are methods for treating inflammation comprising administering to the mammal at least once an effective amount of at least one compound having the structure of Formula (L-I), (L-II), and (I).

In some embodiments, provided herein are methods for the treatment of cancer comprising administering to the mammal at least once an effective amount of at least one compound having the structure of Formula (L-I), (L-II), and (I). The type of cancer may include, but is not limited to, pancreatic cancer and other solid or hematological tumors.

In some embodiments, provided herein are methods for treating respiratory diseases comprising administering to the mammal at least once an effective amount of at least one compound having the structure Formula (L-I), (L-II), and (I). In some embodiments, the respiratory disease is asthma. In some embodiments, the respiratory disease includes, but is not limited to, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, and seasonal asthma.

In some embodiments, provided herein are methods for preventing rheumatoid arthritis and osteoarthritis comprising administering to the mammal at least once an effective amount of at least one compound having the structure of Formula (L-I), (L-II), and (I).

In some embodiments, provided herein are methods for treating inflammatory responses of the skin comprising administering to the mammal at least once an effective amount of at least one compound having the structure of Formula (L-I), (L-II), and (I). Such inflammatory responses of the skin include, by way of example, dermatitis, contact dermatitis, eczema, urticaria, rosacea, and scarring. In another aspect are methods for reducing psoriatic lesions in the skin, joints, or other tissues or organs, comprising administering to the mammal an effective amount of a first compound having the structure of Formula (L-I), (L-II), and (I)

In certain embodiments, provided herein are methods for treating the following diseases or conditions comprising administering to the mammal a compound provided herein. In some embodiments, the disease or condition is ALL (Acute Lymphoblastic Lymphoma), DLBCL (Diffuse Large B-Cell Lymphoma), FL (Follicular Lymphoma), RCC(Renal Cell Carcinoma), Childhoon Medulloblastoma, Glioblastoma, Pancreatic tumor or cancer, Liver cancer (Hepatocellular Carcinoma), Prostate Cancer (Myc), Triple Negative Breast (Myc), AML (Acute Myeloid Leukemia), or MDS (Myelo Dyslplastic Syndrome). In some embodiments, the disease or condition is Early-onset Dystonia. In yet some embodiments, the disease or condition is Kabuki Syndrome.

In some embodiments, the disease or condition is p53 driven tumor. RUNX2 signaling pathway is one of survival signals specific to p53 defective cancer cells. RUNX2 recruits the menin/MLL1 epigenetic complex to induce the expression of MYC. Using small molecule irreversible inhibitors of the menin/MLL1 complex, targeting RUNX2/menin/MLL1/MYC axis is a feasible strategy for killing p53 defective cancer cells (Shih, et al., A RUNX2-Mediated Epigenetic Regulation of the Survival of p53 Defective Cancer Cells. PLOS Genetics, https://doi.org/10.1371/journal.pgen.1005884, 2016).

In some embodiments, the disease or condition is MYC driven tumor. MYC is documented to be involved broadly in many cancers, in which its expression is estimated to be elevated or deregulated in up to 70% of human cancers. High levels of MYC expression have been linked to aggressive human prostate cancer and triple negative breast cancer (Gurel et al., Mod Pathol. 2008 September; 21 (9): 1156-67; Palaskas et al., Cancer Res. 2011 Aug. 1; 71 (15): 5164-74). Experimental models of Myc-mediated tumorigenesis suggest that established tumors are addicted to Myc and that deregulated expression of Myc result in an addiction not only to Myc but also to nutrients. These Myc-induced changes provide a unique opportunity for new therapeutic strategies. Notwithstanding the fact that normal proliferating cells (stem cell compartments and immune cells) also use MYC for renewal, many studies have focused on targeting Myc for cancer therapeutics. Strategies have emerged to inhibit MYC expression, to interrupt Myc-Max dimerization, to inhibit Myc-Max DNA binding, and to interfere with key Myc target genes (Dang et al. Cell. 2012, 149 (1): 22-35).

In another aspect, provided are methods for treating diabetes mellitus comprising administering to the mammal at least once an effective amount of a compound of present invention. In some embodiments, the disease or condition is type 1 diabetes mellitus. In some embodiments, the disease or condition is stage 2 type 1 diabetes mellitus. In some embodiments, the methods delay progression to stage 3 type 1 diabetes mellitus. In some embodiments, the disease or condition is stage 3 type 1 diabetes mellitus. In some embodiments, the methods achieve long-term glycemic control, for instance in stage 3 type 1 diabetes mellitus. In some embodiments, the methods reduce insulin dependency, for instance in stage 3 type 1 diabetes mellitus. In some embodiments, the disease or condition is type 2 diabetes mellitus. In some embodiments, the disease or condition is newly diagnosed type 2 diabetes mellitus. In some embodiments, the disease or condition is gestational diabetes mellitus. In some embodiments, the disease or condition is maturity onset diabetes of the young. In some embodiments, the disease or condition is steroid induced diabetes. In some embodiments, the disease or condition is prediabetes. In some embodiments, the disease or condition is diabetes in a patient at risk for hypoglycemia. In some embodiments, the methods reduce diabetic ketoacidosis. In some embodiments, the methods reduce glucose excursions. In some embodiments, the methods reduce diabetic kidney disease. In some embodiments, the disease or condition is double diabetes.

In any of the aforementioned embodiments are some embodiments in which administration is enteral, parenteral, or both, and wherein (a) an effective amount of a provided compound is systemically administered to the mammal; (b) an effective amount of a provided compound is administered orally to the mammal; (c) an effective amount of a provided compound is intravenously administered to the mammal; (d) an effective amount of a provided compound is administered by inhalation; (e) an effective amount of a provided compound is administered by nasal administration; or (f) an effective amount of a provided compound is administered by injection to the mammal; (g) an effective amount of a provided compound is is administered topically (dermal) to the mammal; (h) an effective amount of a provided compound is administered by ophthalmic administration; or (i) an effective amount of a provided compound is administered rectally to the mammal.

In any of the aforementioned embodiments are some embodiments comprising single administrations of an effective amount of a provided compound, including some embodiments in which (i) a provided compound is administered once; (ii) a provided compound is administered to the mammal multiple times over the span of one day; (iii) continually; or (iv) continuously.

In any of the aforementioned embodiments are some embodiments comprising multiple administrations of an effective amount of a provided compound, including some embodiments in which (i) a provided compound is administered in a single dose; (ii) the time between multiple administrations is every 6 hours; (iii) a provided compound is administered to the mammal every 8 hours. In some embodiments, the method comprises a drug holiday, wherein the administration of the compound is temporarily suspended, or the dose of the compound being administered is temporarily reduced; at the end of the drug holiday, dosing of the compound is resumed. The length of the drug holiday can vary from 2 days to 1 year.

In any of the aforementioned embodiments involving the treatment of proliferative disorders, including cancer, are some embodiments comprising administering at least one additional agent selected from the group consisting of alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, Paclitaxel™, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase irreversible inhibitors such as irinotecan or topotecan, tyrosine kinase irreversible inhibitors such as gefinitinib or imatinib, or agents to treat signs or symptoms induced by such therapy including allopurinol, filgrastim, granisetron/ondansetron/palonosetron, dronabinol.

In some embodiments, the compounds of Formula (L-I), (L-II), and (I) are covalent inhibitors of menin activity. In certain embodiments, such covalent inhibitors have an IC₅₀ below 10 microM in enzyme assay. In some embodiments, a menin inhibitor has an IC₅₀ of less than 1 microM, and in some embodiments, less than 0.25 microM.

Other objects, features, and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### DETAILED DESCRIPTION OF THE PREFERRED EMBODIMENTS

### Certain Terminology

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. Use of the term “including” as well as other forms, such as “include”, “includes,” and “included,” is not limiting. Definition of standard chemistry terms may be found in reference works, including Carey and Sundberg “ADVANCED ORGANIC CHEMISTRY 4TH ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In some embodiments, an alkyl comprises five to fifteen carbon atoms (e.g., C₅-C₁₅ alkyl). In certain embodiments, an alkyl comprises five to eight carbon atoms (e.g., C₅-C₈ alkyl). The alkyl is attached to the rest of the molecule by a single bond, for example, methyl (Me), ethyl (Et), n-propyl (n-pr), 1-methylethyl (iso-propyl or i-Pr), n-butyl (n-Bu), n-pentyl, 1,1-dimethylethyl (t-butyl, or t-Bu), 3-methylhexyl, 2-methylhexyl, and the like. Unless stated otherwise specifically in the specification, an alkyl group is optionally substituted as defined and described below and herein.

The alkyl group could also be a “lower alkyl” having 1 to 6 carbon atoms.

As used herein, C₁-Cₓ includes C₁-C₂, C₁-C₃ . . . C₁-Cₓ

“Alkenyl” refers to a straight or branched hydrocarbon chain radical group consisting solely of carbon and hydrogen atoms, containing at least one double bond, and having from two to twelve carbon atoms. In certain embodiments, an alkenyl comprises two to eight carbon atoms. In some embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted as defined and described below and herein.

“Alkynyl” refers to a straight or branched hydrocarbon chain radical group consisting solely of carbon and hydrogen atoms, containing at least one triple bond, having from two to twelve carbon atoms. In certain embodiments, an alkynyl comprises two to eight carbon atoms. In some embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted as defined and described below and herein.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. Unless stated otherwise specifically in the specification, an alkylene chain is optionally substituted as defined and described below and herein.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, n-butenylene, and the like. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain is optionally substituted as defined and described below and herein. “Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl (Ph), fluorenyl, and naphthyl. Unless stated otherwise specifically in the specification, the term “aryl” or the prefix “ar-” (such as in “aralkyl”) is meant to include aryl radicals optionally substituted as defined and described below and herein.

“Aralkyl” refers to a radical of the formula —R^{c}-aryl where R^{c} is an alkylene chain as defined above, for example, benzyl, diphenylmethyl and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —Re-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In some embodiments, a carbocyclyl comprises five to seven carbon atoms. The carbocyclyl is attached to the rest of the molecule by a single bond. Carbocyclyl is optionally saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted as defined and described below and herein. “Halo” or “halogen” refers to bromo, chloro, fluoro or iodo substituents.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In some embodiments in which two or more hydrogen atoms are replaced with halogen atoms, the halogen atoms are not all the same as one another.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical is optionally substituted as defined above for an alkyl group.

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three to 14 ring atoms, such as three, four, five, six, seven, eight, nine, or more than nine atoms. A “non-aromatic heterocycle” or “heterocycloalkyl” can be saturated or unsaturated. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, azetidine. oxetane, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

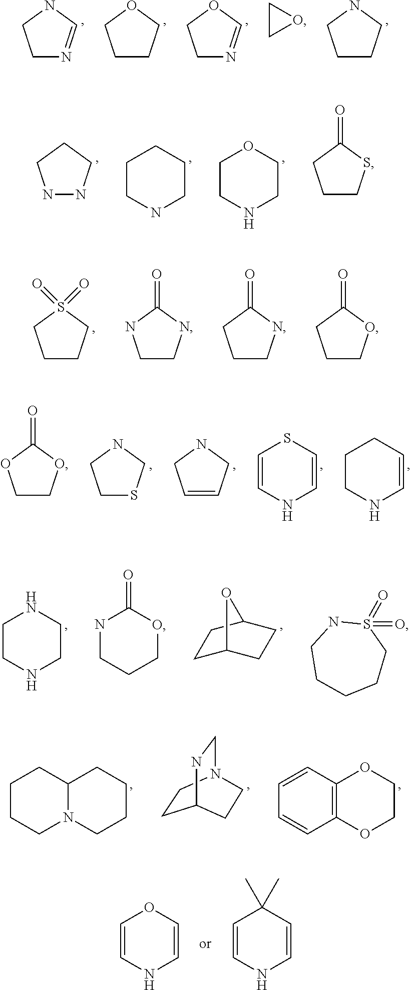

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group). The term heterocycloalkyl also includes fused, bicyclics, tricyclics, bridged, spiro and other ring forms.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. In some embodiments, heteroaryl rings have five, six, seven, eight, nine, or more than nine ring atoms. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl,isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted as defined and described below and herein.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Sulfanyl” refers to the —S-radical.

“Sulfinyl” refers to the —S(═O)— radical.

“Sulfonyl” refers to the —S(═O)₂— radical.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Imino” refers to the ═NH radical.

“Thioxo” refers to the ═S radical.

An “alkoxy” group refers to a (alkyl)O— group, where alkyl is as defined herein.

An “aryloxy” group refers to an (aryl)O— group, where aryl is as defined herein.

“Carbocyclylalkyl” means an alkyl radical, as defined herein, substituted with a carbocyclyl group. “Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

As used herein, the terms “heteroalkyl” “heteroalkenyl” and “heteroalkynyl” include optionally substituted alkyl, alkenyl and alkynyl radicals in which one or more skeletal chain atoms is a heteroatom, e.g., oxygen, nitrogen, sulfur, silicon, boron, phosphorus or combinations thereof. The heteroatom(s) may be placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃) 3, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond,” “direct bond” or “single bond” refers to a chemical bond between two atoms, or two moieties when the atoms joined by the bond are considered to be part of larger substructure.

An “isocyanato” group refers to a —NCO group.

An “isothiocyanato” group refers to a —NCS group.

The term “moiety” refers to a specific segment or functional group of a molecule. Chemical moieties are often recognized chemical entities embedded in or appended to a molecule.

A “thioalkoxy” or “alkylthio” group refers to a —S-alkyl group.

A “alkylthioalkyl” group refers to an alkyl group substituted with a —S-alkyl group.

As used herein, the term “acyloxy” refers to a group of formula RC(═O)O—.

“Carboxy” means a-C(O) OH radical.

As used herein, the term “acetyl” refers to a group of formula —C(═O) CH₃. “Acyl” refers to the group-C(O)R.

As used herein, the term “trihalomethanesulfonyl” refers to a group of formula X₃CS(═O)₂—where X is a halogen.

“Cyanoalkyl” means an alkyl radical, as defined herein, substituted with at least one cyano group.

As used herein, the term “N-sulfonamido” or “sulfonylamino” refers to a group of formula RS(═O) 2NH—.

As used herein, the term “O-carbamyl” refers to a group of formula —OC(═O)NR₂.

As used herein, the term “N-carbamyl” refers to a group of formula ROC(═O)NH—.

As used herein, the term “O-thiocarbamyl” refers to a group of formula —OC(═S) NR₂.

As used herein, “N-thiocarbamyl” refers to a group of formula ROC(═S)NH—.

As used herein, the term “C-amido” refers to a group of formula —C(═O)NR₂.

“Aminocarbonyl” refers to a —CONH₂ radical.

As used herein, the term “N-amido” refers to a group of formula RC(═O)NH—.

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

An “alkenyloxy” group refers to a (alkenyl)O— group, where alkenyl is as defined herein.

The term “alkylamine” refers to the —N(alkyl),Hy group, where x and y are selected from among x=1, y=1 and x=2, y=0. When x=2, the alkyl groups, taken together with the N atom to which they are attached, can optionally form a cyclic ring system.

“Alkylaminoalkyl” refers to an alkyl radical, as defined herein, substituted with an alkylamine, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3rd Ed., John Wiley & Sons, New York, NY, 1999, which is incorporated herein by reference in its entirety.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

As described herein, compounds provided herein may be “optionally substituted”. In general, the term “substituted,” whether preceded by the term “optionally” or not, means that one or more hydrogens of a designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents provided herein are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)⁰⁻⁴R^{o}; —(CH₂)₀₋₄OR^{o}; —O(CH₂)₀₋₄R^{o}, —O—(CH₂)₀₋₄C(O) OR^{o}; —(CH₂)₀₋₄ CH(OR^{o})₂; —(CH₂)₀₋₄SR^{o}; —(CH₂)₀₋₄Ph, which may be substituted with R^{o}; —(CH₂)₀₋₄ C(CH₂)₀₋₁Ph which may be substituted with R^{o}; —CH—CHPh, which may be substituted with R^{o}; —(CH₂)₀₋₄O(CH₂)₀₋₁-pyridyl which may be substituted with R^{o}; —NO₂; —CN; —N₃); (CH₂)₀₋₄N(R^{o})₂; —(CH₂)₀₋₄N(R^{o})C(O)R^{o}; —N(R^{6c} (S)R^{o}; —(CH₂)₀₋₄ N(R^{o})C(O)NR₂^{o}; —N(R^{o})C(S)NR₂^{o}; —(CH₂)₀₋₄N(R^{o})(O)OR^{o};
N(R^{o})N(R^{o})C(O)R^{o}; —N(R^{o}N(R^{o})C(O)NR₂^{o}; —N(R^{o})N(R^{o})C(O)OR^{o}; —(CH₂)₀₋₄C(O)R^{o}; —C(S)R^{o}; —(CH₂)₀₋₄ C(O) OR^{o}; —(CH₂)₀₋₄C(O) SR^{o}; —(CH₂)₀₋₄C(O)OSiR₃^{o}; —(CH₂)₀₋₄OC(O)R^{o}; —OC(O)(CH₂)₀₋₄SR—, —SC(S)SR^{o}; —(CH₂)₀₋₄SC(O)R^{o}; —(CH₂)₀₋₄C(O) NR₂^{o}; —C(S) NR₂^{o}; —C(S) SR^{o}; —(CH₂)₀₋₄OC(O) NR₂^{o}; —C(O) N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR₂^{o}; —(CH₂)₀₋₄S(O)R^{o}; —N(R^{o}S(O)₂NR₂^{o}; —N(R^{o})S(O)—R^{o}; —N(OR^{o})R^{o}; —C(NH) NR₂^{o}; —P(O)₂R^{o}; —P(O)R₂^{o}; —OP(O)R₂^{o}; —OP(O)(OR^{o})₂; SiR₃^{o}; —(C₁₋₄ straight or branched alkylene)O—) N(R^{o})₂; or —(C₁₋₄ straight or branched) alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•})-(haloR^{•}, —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}, —NO₂, —SiR₃^{•}, —OSiR₃^{•}, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃₀—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃₀—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•})-(haloR^{•}, —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include-Rt, —NR₂^{†}, —C(O)R^{†}, —C(O)OR^{†}, —C(O)C(O)R^{†}, —C(O)CH₂C(O)R^{†}, —S(O)₂R^{†}, —S(O)—NR*₂, —C(S)NR₂^{†}, —C(NH)NR₂^{†}, or —N(R^{†})S(O)₂R^{†}; wherein each R^{t} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{•}-(haloR^{•}), —OH, —OR^{•}, —O(haloR^{•}), —CN, —C(O)OH, —C(O)OR, —NH₂, —NHR^{•}, —NR₂^{•}, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “nucleophile” or “nucleophilic” refers to an electron rich compound, or moiety thereof.

The term “electrophile”, or “electrophilic” refers to an electron poor or electron deficient molecule, or moiety thereof. Examples of electrophiles include, but in no way are limited to, Michael acceptor moieties.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

As used herein, “amelioration” of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

“Bioavailability” refers to the percentage of the weight of compounds disclosed herein, such as, compounds of any of formula described herein or Formula (L-I), (L-II), and (I) dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which compounds disclosed herein, such as, compounds of any of formula described herein or Formula (L-I), (L-II), and (I) are absorbed into the general circulation when the pharmaceutical composition is taken orally as compared to intravenous injection.

“Blood plasma concentration” refers to the concentration of compounds disclosed herein, such as, compounds of any of formula described herein or Formula (L-I), (L-II), and (I) in the plasma component of blood of a subject. It is understood that the plasma concentration of compounds of any of formula described herein or Formula (L-I), (L-II), and (I) may vary significantly between subjects, due to variability with respect to metabolism and/or possible interactions with other therapeutic agents. In accordance with some embodiments disclosed herein, the blood plasma concentration of the compounds of any of formula described herein or Formula (L-I), (L-II), and (I) may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound of any of formula described herein or Formula (L-I), (L-II), and (I) may vary from subject to subject.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound of any of Formula (L-I), (L-II), and (I), age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The term “identical,” as used herein, refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using comparison algorithms or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence. By way of example only, two or more polypeptide sequences are identical when the amino acid residues are the same, while two or more polypeptide sequences are “substantially identical” if the amino acid residues are about 60% identical, about 65% identical, about 70% identical, about 75% identical, about 80% identical, about 85% identical, about 90% identical, or about 95% identical over a specified region. The identity can exist over a region that is at least about 75-100 amino acids in length, over a region that is about 50 amino acids in length, or, where not specified, across the entire sequence of a polypeptide sequence. In addition, by way of example only, two or more polynucleotide sequences are identical when the nucleic acid residues are the same, while two or more polynucleotide sequences are “substantially identical” if the nucleic acid residues are about 60% identical, about 65% identical, about 70% identical, about 75% identical, about 80% identical, about 85% identical, about 90% identical, or about 95% identical over a specified region. The identity can exist over a region that is at least about 75-100 nucleic acids in length, over a region that is about 50 nucleic acids in length, or, where not specified, across the entire sequence of a polynucleotide sequence.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

The term “irreversible inhibitor,” or “covalent inhibitor,” as used herein, refers to a compound that, upon contact with a target protein (e.g., menin) causes the formation of a stable covalent bond with or within the protein. In certain embodiments, one or more of the target protein's biological activities (e.g., phosphotransferase activity) is diminished or abolished notwithstanding the subsequent presence or absence of the irreversible inhibitor. In certain embodiments, the covalent bond is not reversible under physiological conditions. In certain embodiments, the covalent bond is not reversible in vivo. In contrast, a reversible inhibitor compound upon contact with a target protein does not cause the formation of a new covalent bond with or within the protein and therefore can associate and dissociate from the target poteen.

The term “irreversible inhibitor of menin” as used herein, refers to an inhibitor of menin that can form a covalent bond with an amino acid residue of menin. In one embodiment, the irreversible inhibitor of menin can form a covalent bond with a Cys residue of menin; in particular embodiments, the irreversible inhibitor can form a covalent bond with a Cys 329 residue (or a homolog thereof) of menin.

The term “prophylactically effective amount,” as used herein, refers that amount of a composition applied to a patient that will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

As used herein, the term “selective binding compound” refers to a compound that selectively binds to any portion of one or more target proteins.

As used herein, the term “selectively binds” refers to the ability of a selective binding compound to bind to a target protein, such as, for example, menin, with greater affinity than it binds to a non-target protein. In certain embodiments, specific binding refers to binding to a target with an affinity that is at least 10, 50, 100, 250, 500, 1000 or more times greater than the affinity for a non-target.

As used herein, the term “selective modulator” refers to a compound that selectively modulates a target activity relative to a non-target activity. In certain embodiments, specific modulater refers to modulating a target activity at least 10, 50, 100, 250, 500, 1000 times more than a non-target activity.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

The term “subject” or “patient” as used herein, refers to an animal which is the object of treatment, observation, or experiment. By way of example only, a subject may be, but is not limited to, a mammal including, but not limited to, a human.

As used herein, the term “target activity” refers to a biological activity capable of being modulated by a selective modulator. Certain exemplary target activities include, but are not limited to, binding affinity, signal transduction, enzymatic activity, tumor growth, inflammation, or inflammation-related processes, and amelioration of one or more symptoms associated with a disease or condition.

As used herein, the term “target protein” refers to a molecule or a portion of a protein capable of being bound by a selective binding compound. In certain embodiments, a target protein is menin.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the IC₅₀ refers to an amount, concentration, or dosage of a particular test compound that achieves a 50% inhibition of a maximal response, such as inhibition of menin or menin-MLL, in an assay that measures such response.

As used herein, EC₅₀ refers to a dosage, concentration, or amount of a particular test compound that elicits a dose-dependent response at 50% of maximal expression of a particular response that is induced, provoked or potentiated by the particular test compound.

Methods described herein include administering to a subject in need a composition containing a therapeutically effective amount of one or more menin or menin-MLL inhibitor compounds described herein.

In some embodiments, methods described herein can be used to treat an autoimmune disease, which includes, but is not limited to, rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitis, antiphospholipid antibody syndrome, aplastic anemia, autoimmune hepatitis, coeliac disease, Goodpasture's syndrome, idiopathic thrombocytopenia purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, and vulvodynia.

In some embodiments, methods described herein can be used to treat heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

In some embodiments, methods described herein can be used to treat an inflammatory disease, which includes, but is not limited to asthma, inflammatory bowel disease, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, and vulvitis.

In some embodiments, methods described herein can be used to treat a cancer, e.g., B-cell proliferative disorders, which include, but are not limited to diffuse large B cell lymphoma, follicular lymphoma, chronic lymphocytic lymphoma, chronic lymphocytic leukemia, B-cell prolymphocytic leukemia, lymphoplasmacytic lymphoma/Waldenström macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, and lymphomatoid granulomatosis.

In some embodiments, methods described herein can be used to treat thromboembolic disorders, which include, but are not limited to myocardial infarct, angina pectoris (including unstable angina), reocclusions or restenoses after angioplasty or aortocoronary bypass, stroke, transitory ischemia, peripheral arterial occlusive disorders, pulmonary embolisms, and deep venous thromboses.

Symptoms, diagnostic tests, and prognostic tests for each of the above-mentioned conditions are known in the art. See, e.g., Harrison's Principles of Internal Medicine©,” 16th ed., 2004, The McGraw-Hill Companies, Inc. Dey et al. (2006), Cytojournal 3 (24), and the “Revised European American Lymphoma” (REAL) classification system (see, e.g., the website maintained by the National Cancer Institute).

A number of animal models of are useful for establishing a range of therapeutically effective doses of menin inhibitor compounds for treating any of the foregoing diseases.

For example, dosing of menin inhibitor compounds for treating an autoimmune disease can be assessed in a mouse model of rheumatoid arthritis. In this model, arthritis is induced in Balb/c mice by administering anti-collagen antibodies and lipopolysaccharide. See Nandakumar et al. (2003), Am. J. Pathol 163:1827-1837.

In another example, dosing of menin irreversible inhibitors for the treatment of B-cell proliferative disorders can be examined in, e.g., a human-to-mouse xenograft model in which human B-cell lymphoma cells (e.g. Ramos cells) are implanted into immunodeficient mice (e.g., “nude” mice) as described in, e.g., Pagel et al. (2005), Clin Cancer Res 11 (13): 4857-4866.

Animal models for treatment of thromboembolic disorders are also known.

The therapeutic efficacy of a provided compound for one of the foregoing diseases can be optimized during a course of treatment. For example, a subject being treated can undergo a diagnostic evaluation to correlate the relief of disease symptoms or pathologies to inhibition of in vivo menin activity achieved by administering a given dose of a menin inhibitor.

### Compounds

In the following description of menin inhibitor compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employedUnless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

Menin inhibitor compounds can be used for the manufacture of a medicament for treating any of the foregoing conditions (e.g., autoimmune diseases, inflammatory diseases, allergy disorders, B-cell proliferative disorders, Myeloid cell proliferative disorder, Lymphoid cell proliferative disorder, or thromboembolic disorders).

In some embodiments, the menin inhibitor compound used for the methods described herein inhibits menin activity with an in vitro IC₅₀ of less than about 10 μM (e.g., less than about 1 μM, less than about 0.5 μM, less than about 0.4 μM, less than about 0.3 μM, less than about 0.1 μM, less than about 0.08 μM, less than about 0.06 μM, less than about 0.05 μM, less than about 0.04 μM, less than about 0.03 μM, less than about 0.02 μM, less than about 0.01 μM, less than about 0.008 μM, less than about 0.006 μM, less than about 0.005 μM, less than about 0.004 μM, less than about 0.003 μM, less than about 0.002 μM, less than about 0.001 μM, less than about 0.00099 μM, less than about 0.00098 μM, less than about 0.00097 μM, less than about 0.00096 μM, less than about 0.00095 μM, less than about 0.00094 μM, less than about 0.00093 μM, less than about 0.00092 μM, or less than about 0.00090 μM).

In some embodiments, the menin inhibitor compound selectively inhibits an activated form of its target menin.

Also described herein are methods for synthesizing such irreversible inhibitors, methods for using such irreversible inhibitors in the treatment of diseases (including diseases wherein inhibition of menin interaction provides therapeutic benefit to a patient having the disease). Further described are pharmaceutical compositions that include an inhibitor of menin interaction. Specifically, described herein are compounds and methods of use thereof to inhibit interaction of menin with MLL oncoproteins (e.g., MLL1, MLL2, MLL-fusion oncoproteins).

Specifically described herein are irreversible inhibitors of menin interaction that form a covalent bond with a cysteine residue on menin. Further described herein are irreversible inhibitors of menin interaction that form a covalent bond with a Cys329 residue on menin. Also described are pharmaceutical formulations that include a irreversible inhibitor of menin.

The menin inhibitor compounds described herein are selective for menin having a cysteine residue in an amino acid sequence position of the menin protein that is homologous to the amino acid sequence position of cysteine 329 in menin. Irreversible inhibitor compounds described herein include a Michael acceptor moiety.

Generally, a reversible or irreversible inhibitor compound of menin used in the methods described herein is identified or characterized in an in vitro assay, e.g., an acellular biochemical assay or a cellular functional assay. Such assays are useful to determine an in vitro IC₅₀ for a reversible or irreversible menin inhibitor compound.

Further, covalent complex formation between menin and a candidate irreversible menin inhibitor is a useful indicator of irreversible inhibition of menin that can be readily determined by a number of methods known in the art (e.g., mass spectrometry). For example, some irreversible menin-inhibitor compounds can form a covalent bond with Cys 329 of menin (e.g., via a Michael reaction). See S. Xu et al. Angewandte Chemie International Ed. 57 (6), 1601-1605 (2017) (incorporated by reference in its entirety).

Described herein are compounds of any of Formulae (L-I), (L-II), and (I). Also described herein are pharmaceutically acceptable salts, pharmaceutically acceptable solvates, pharmaceutically active metabolites, and pharmaceutically acceptable prodrugs of such compounds. Pharmaceutical compositions that include at least one such compound or a pharmaceutically acceptable salt, pharmaceutically acceptable solvate, pharmaceutically active metabolite or pharmaceutically acceptable prodrug of such compound, are provided. In some embodiments, when compounds disclosed herein contain an oxidizable nitrogen atom, the nitrogen atom can be converted to an N-oxide by methods well known in the art. In certain embodiments, isomers and chemically protected forms of compounds having a structure represented by any of Formula (L-I), (L-II), and (I) are also provided.

In some embodiments, provided herein are menin or menin-MLL covalent inhibitors according to compounds of formula (L-I). In some embodiments, provided herein are menin covalent inhibitors according to compounds of formula (L-I). In some embodiments, provided herein are menin covalent inhibitors according to compounds of formula (L-I). In some embodiments, provided herein are menin covalent inhibitors according to compounds of formula (L-I).

In some embodiments, provided herein is a compound according to Formula (L-I) having the structure:

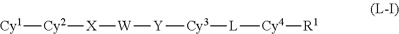

or a stereoisomer thereof; or a pharmaceutically acceptable salt thereof,
wherein:
Cy¹ is substituted or unsubstituted

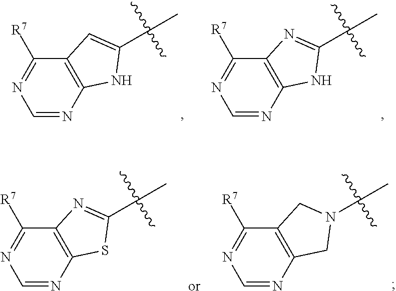

and the substitution on Cy¹ is C₁-C₄ alkyl, CN, or halo;
Cy² is substituted or unsubstituted cycloalkylene, substituted or unsubstituted heterocycloalkylene, substituted or unsubstituted arylene, or substituted or unsubstituted heteroarylene;
X is —NR^{3a}—, —C(R^{3b})₂—, or —O—;
W is —C(R^{3b})₂—, —C(O)—, —S(O)—, or —S(O)₂—;
Y is absent, —NR^{3a}—, —C(R^{3b})₂—, or —O—;
or X—W—Y is —N(H)—, —O—, or —S(O)₂—N(H)—C(R^{3b})₂—;
Cy³ is substituted or unsubstituted cycloalkylene, substituted or unsubstituted heterocycloalkylene, substituted or unsubstituted arylene, or substituted or unsubstituted heteroarylene;
L is a single bond, substituted or unsubstituted —N(H)—, —C(F)₂—O—, or substituted or unsubstituted C₁₋₄ alkylene; the substitution on alkylene is C₁-C₄ alkyl, CN, or halo; and the substitution on-N(H)— is C₁-C₄ alkyl;
i)R¹ is —B-C(R^{6a})—C(R^{6b})—C(O)—R^{6c}, —B—C(R^{6a})═C(R^{6b})—S(O)—R^{6c}, —B—C(R^{6a})═C(R^{6b})—S(O)₂—R^{6c}, —B—C(R^{6a})—C(R^{6b})—P(O)—R^{6a}R^{6b}; or —B—C(R^{6a})—C(R^{6b})—P(O)—OR^{6a}OR^{6b}, B is substituted or unsubstituted C₁₋₄ alkylene; R^{6c} is substituted or unsubstituted alkoxy, substituted or unsubstituted amino, or substituted or unsubstituted heterocycloalkyl, having 1-2 heteroatoms independently selected from nitrogen, oxygen, and sulfur; and Cy⁴ is absent, substituted or unsubstituted cycloalkylene, substituted or unsubstituted heterocycloalkylene, substituted or unsubstituted arylene, or substituted or unsubstituted heteroarylene; each R^{6a} and R^{6b} is independently H, CN, halo, or substituted or unsubstituted C₁₋₆ alkyl; and the substitution on C₁₋₆ alkyl is 1, 2, or 3 groups independently selected from halo, alkoxy, alkylamino, dialkylamino, and heterocycloalkyl;
ii)R¹ is —C(O)—C(R^{6a})═C(R^{6b})R^{6c}, —S(O)—C(R^{6a})═C(R^{6b})R^{6c}, —S(O)₂—C(R^{6a})═C(R^{6b})R^{6c}, —NR^{3c}—C(O)—C(R^{6a})═C(R^{6b})R^{6c}, —NR^{3c}—S(O)—C(R^{6a})═C(R^{6b})R^{6c}, or —NR^{3c}—S(O)₂—C(R^{6a})═C(R^{6b})R^{6c}; and Cy⁴ is absent;

iii)R¹ is —C(O)—C(R^{6a})═C(R^{6b})R^{6c}, —S(O)—C(R^{6a})═C(R^{6b})R⁶, —S(O)₂—C(R^{6a})═C(R^{6b})R^{6c}, —NR^{3c}—C(O)—C(R^{6a})═C(R^{6b})R^{6c}, —NR^{3c}—S(O)—C(R^{6a})—C(R^{6b})R^{6c}, or —NR^{3c}—S(O)₂—C(R^{6a})═C(R^{6b})R^{6c}; Cy⁴ is absent, substituted or unsubstituted cycloalkylene, substituted or unsubstituted heterocycloalkylene, substituted or unsubstituted arylene, or substituted or unsubstituted heteroarylene; and R^{6a} is alkyl, substituted with halo, alkoxy, alkylamino, dialkylamino, or heterocycloalkyl;
iv)R¹ is —C(O)—C(R^{6a})—C(R^{6b})R^{6c}, —S(O)—C(R^{6a})═C(R^{6b})R^{6c}, —S(O)₂—C(R^{6a})—C(R^{6b})R^{6c}, —NR^{3c}—C(O)—C(R^{6a})—C(R^{6b})R^{6c}, —NR^{3c}—S(O)—C(R^{6a})—C(R^{6b})R^{6c}, or —NR^{3c}—S(O)₂—C(R^{6a})—C(R^{6b})R^{6c}; Cy⁴ is substituted or unsubstituted azetidinylene, substituted or unsubstituted pyrrolidinylene, substituted or unsubstituted piperidinylene, or substituted or unsubstituted piperazinylene; and the substitution on each azetidinylene, pyrrolidinylene, piperidinylene, and piperazinylene is 1, 2, or 3 groups independently selected from halo, CN, hydroxy, substituted or unsubstituted C₁-C₄ alkyl, and substituted or unsubstituted C₁-C₄ alkoxy
or
v)R¹ is

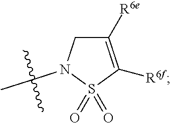

Cy⁴ is absent, substituted or unsubstituted azetidinylene, substituted or unsubstituted pyrrolidinylene, substituted or unsubstituted piperidinylene, or substituted or unsubstituted piperazinylene;
each R^{3a}, R^{3b}, and R^{3c} is independently H or substituted or unsubstituted C₁₋₄ alkyl; and the substitution on each azetidinylene, pyrrolidinylene, piperidinylene, and piperazinylene is 1, 2, or 3 groups independently selected from halo, CN, hydroxy, substituted or unsubstituted C₁-C₄ alkyl, and substituted or unsubstituted C₁-C₄ alkoxy
each R^{6a} and R^{6b} is independently H, CN, halo, or substituted or unsubstituted C₁₋₆ alkyl; or R^{6a} and R^{6b} are joined together to form a bond;
R^{6c} is substituted or unsubstituted alkoxy, substituted or unsubstituted amino, substituted or unsubstituted heterocycloalkyl, having 1-2 heteroatoms independently selected from nitrogen, oxygen, and sulfur; each R^{6e} and R^{6f} is independently H, CN, halo, and C₁₋₆ alkyl; and the substitution on heterocycloalkyl is 1, 2, or 3 groups independently selected from C₁-C₄ alkyl, hydroxy, CN, C₁-C₄ alkoxy, and halo; and each substitution on amino and alkoxy is independently C₁-C₄ alkyl; and
R⁷ is a 4-10 membered substituted or unsubstituted heterocycloalkyl ring having 1-2 heteroatoms independently selected from nitrogen, oxygen, and sulfur, substituted or unsubstituted phenyl, an 8-10 membered substituted or unsubstituted bicyclic aryl ring, and a 5-6 membered substituted or unsubstituted heteroaryl ring having 1-4 heteroatoms independently selected from nitrogen, oxygen, and sulfur; and the substitution on the phenyl, and the bicyclic aryl is 1, 2, or 3 groups independently selected from C₁-C₄ alkyl, hydroxy, CN, C₁-C₄ alkoxy; and wherein the substitution on each alkyl is 1, 2, or 3 groups independently selected from halo, CN, hydroxy, and C₁-C₄ alkoxy; the substitution on each alkoxy is 1, 2, or 3 groups independently selected from halo, CN, and substituted or unsubstituted C₁-C₄ alkyl; the substitution on each cycloalkyl is 1, 2, or 3 groups independently selected from halo, CN, hydroxy, substituted or unsubstituted C₁-C₄ alkyl, and substituted or unsubstituted C₁-C₄ alkoxy; the substitution on each heterocycloalkyl is 1, 2, or 3 groups independently selected from halo, CN, hydroxy, substituted or unsubstituted C₁-C₄ alkyl, and substituted or unsubstituted C₁-C₄ alkoxy; the substitution on each aryl is 1, 2, or 3 groups independently selected from halo, haloalkyl, amino, dialkylamino, amido, CN, hydroxy, substituted or unsubstituted C₁-C₄ alkyl, and substituted or unsubstituted C₁-C₄ alkoxy; the substitution on each heteroaryl is 1-3 groups independently selected from halo, haloalkyl, amino, dialkylamino, amido, CN, hydroxy, substituted or unsubstituted C₁-C₄ alkyl, substituted or unsubstituted C₁-C₄ alkoxy, and oxo;
provided the compound is according to formula LXIa, LXIb, LXII, LXIII, LXIV, LXV, or LXVI;
or the compound is any one of the compounds listed in Table 1G, 1H, 1J, and 1K.

In some embodiments, provided herein is a compound according to Formula (L-I) having the structure:

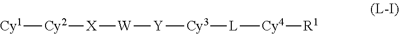

or a stereoisomer thereof; or a pharmaceutically acceptable salt thereof,
wherein:
Cy¹ is substituted or unsubstituted

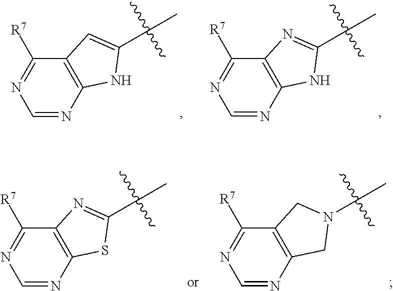

Cy² is substituted or unsubstituted

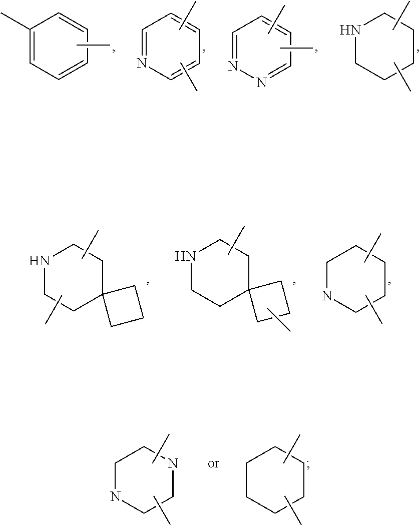

X is —NR^{3a}—, —C(R^{3b})₂—, or —O—;
W is —C(R^{3b})₂—, —C(O)—, —S(O)—, or —S(O)₂—;
Y is absent, —NR^{3a}—, —C(R^{3b})₂—, or —O—;
or X—W—Y is —N(H)—, —O—, or —S(O)₂—N(H)—C(R^{3b})₂—;
Cy³ is substituted or unsubstituted phenylene, pyridylene, pyrimidinylene, substituted or unsubstituted azetidinylene, substituted or unsubstituted pyrrolidinylene, substituted or unsubstituted piperidinylene, or substituted or unsubstituted piperazinylene;
L is a single bond, substituted or unsubstituted —N(H)—, —C(F)₂—O—, or substituted or unsubstituted C₁₋₄ alkylene;
Cy⁴ is absent, substituted or unsubstituted azetidinylene, substituted or unsubstituted pyrrolidinylene, substituted or unsubstituted piperidinylene, or substituted or unsubstituted piperazinylene;
R¹ is —C(O)—B—C(R^{6a})═C(R^{6b})—C(O)—R^{6c}, —S(O)—B—C(R^{6a})═C(R^{6b})—C(O)—R^{6c}, —S(O)₂—B—C(R^{6a})═C(R^{6b})—C(O)—R^{6c}, —B—C(R^{6a})═C(R^{6b})—C(O)—R^{6c}, —B—C(R^{6a})═C(R^{6b})—S(O)—R^{6c}, —B—C(R^{6a})—C(R^{6b})—S(O)₂—R^{6c}, —B—C(R^{6a})—C(R^{6b})—P(O)—R^{6a}R^{6b}; or —B—C(R^{6a})—C(R^{6b})—P(O)—OR^{6a}OR^{6b}; B is substituted or unsubstituted C₁₋₄ alkylene, substituted or unsubstituted cycloalkyl, or substituted or unsubstituted heterocycloalkyl; each R^{3a}, R^{3b}, and R^{3c} is independently H or substituted or unsubstituted C₁₋₄ alkyl;
each R^{6a} and R^{6b} is independently H, CN, halo, or C₁₋₆ alkyl; or R^{6a} and R^{6b} are joined together to form a bond; R^{6c} is hydroxy, substituted or unsubstituted alkoxy, substituted or unsubstituted amino, substituted or unsubstituted heterocycloalkyl, having 1-2 heteroatoms independently selected from nitrogen, oxygen, and sulfur;
R⁷ is H, an optionally substituted group selected from alkyl, a 4-10 membered heterocycloalkyl ring having 1-2 heteroatoms independently selected from nitrogen, oxygen, and sulfur, phenyl, an 8-10 membered bicyclic aryl ring, and a 5-6 membered heteroaryl ring having 1-4 heteroatoms independently selected from nitrogen, oxygen, and sulfur.

In some embodiments, provided herein is a compound according to formula (L-II):

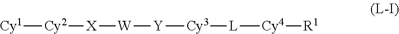

or a stereoisomer thereof; or a pharmaceutically acceptable salt thereof,
wherein:
Cy¹ is substituted or unsubstituted

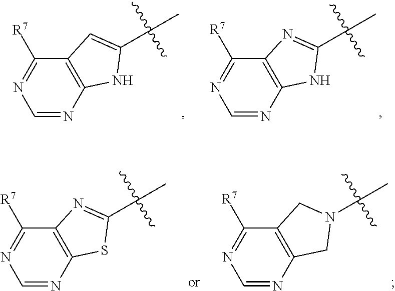

Cy² is substituted or unsubstituted

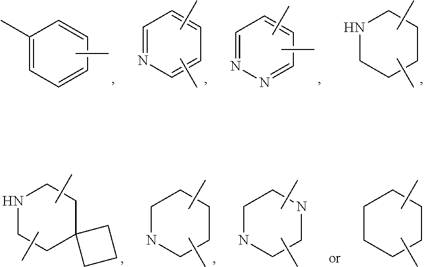

X is —NR^{3a}—, —C(R^{3b})₂—, or —O—;
W is —C(R^{3b})₂—, —C(O)—, —S(O)—, or —S(O)₂—;
Y is absent, —NR^{3a}—, —C(R^{3b})₂—, or —O—;
or X—W—Y is —N(H)—, —O—, or —S(O)₂—N(H)—C(R^{3b})₂—;
Cy³ is substituted or unsubstituted phenylene, pyridylene, pyrimidinylene, or piperidinylene;
L is a single bond, substituted or unsubstituted —N(H)—, —C(F)₂—O—, substituted or unsubstituted C₁₋₄ alkylene, substituted or unsubstituted C₂₋₄ alkenylene, substituted or unsubstituted C₂₋₄ alkynylene, —C(O)—, substituted or unsubstituted cyclopropylene, substituted or unsubstituted cyclobutylene;
Cy⁴ is absent, substituted or unsubstituted azetidinylene, substituted or unsubstituted pyrrolidinylene, substituted or unsubstituted piperidinylene, or substituted or unsubstituted piperazinylene; R¹ is —C(O)—B—C(R^{6a})—C(R^{6b})—C(O)—R^{6c}, —S(O)—B—C(R^{6a})—C(R^{6b})—C(O)—R^{6c}, —S(O)₂—B—C(R^{6a})═C(R^{6b})—C(O)—R^{6c}, —B—C(R^{6a})═C(R^{6b})—C(O)—R^{6c}, —B—C(R^{6a})—C(R^{6b})—S(O)—R^{6c}; —B-C(R^{6a})═C(R^{6b})—S(O)₂—R^{6c}; B is substituted or unsubstituted C₁₋₄ alkylene;
R¹ is —C(O)—B—C(R^{6a})═C(R^{6b})—C(O)—R^{6c}, —S(O)—B—C(R^{6a})—C(R^{6b})—C(O)—R^{6c}, —S(O)₂—B—C(R^{6a})═C(R^{6b})—C(O)—R^{6c}, —B—C(R^{6a})═C(R^{6b})—C(O)—R^{6c}, —B—C(R^{6a})—C(R^{6b})—S(O)—R^{6c}, —B—C(R^{6a})—C(R^{6b})—S(O)₂—R^{6c}, —B—C(R^{6a})—C(R^{6b})—P(O)—R^{6a}R^{6b}; or —B—C(R^{6a})═C(R^{6b})—P(O)—OR^{6a}OR^{6b}; B is substituted or unsubstituted C₁₋₄ alkylene, substituted or unsubstituted cycloalkyl, or substituted or unsubstituted heterocycloalkyl; each R^{3a}, R^{3b}, and R^{3c} is independently H or substituted or unsubstituted C₁₋₄ alkyl;
each R^{6a} and R^{6b} is independently H, CN, halo, or C₁₋₆ alkyl; or R^{6a} and R^{6b} are joined together to form a bond; R^{6c} is hydroxy, substituted or unsubstituted alkoxy, substituted or unsubstituted amino, substituted or unsubstituted heterocycloalkyl, having 1-2 heteroatoms independently selected from nitrogen, oxygen, and sulfur;
R⁷ is H, an optionally substituted group selected from alkyl, a 4-10 membered heterocycloalkyl ring having 1-2 heteroatoms independently selected from nitrogen, oxygen, and sulfur, phenyl, an 8-10 membered bicyclic aryl ring, and a 5-6 membered heteroaryl ring having 1-4 heteroatoms independently selected from nitrogen, oxygen, and sulfur.

In another aspect, provided herein is a compound according to Formula (L-I) having the structure:

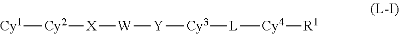

or a stereoisomer thereof; or a pharmaceutically acceptable salt thereof,
wherein
Cy¹ is substituted or unsubstituted

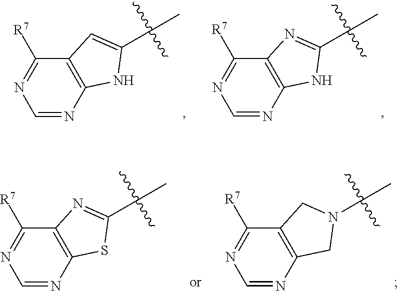

and the substitution on Cy¹ is C₁-C₄ alkyl, CN, or halo;
Cy² is substituted or unsubstituted

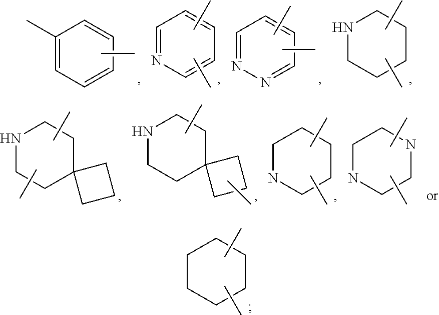

and the substitution on Cy² is selected from 1, 2, or 3 groups independently selected from C₁-C₄ alkyl, hydroxy, CN, C₁-C₄ alkoxy, and halo;
X is —NR^{3a}—, —C(R^{3b})₂—, or —O—;
W is —C(R^{3b})₂—, —C(O)—, —S(O)—, or —S(O)₂—;
Y is absent, —NR^{3a}—, —C(R^{3b})₂—, or —O—;
or X—W—Y is —N(H)—, —O—, or —S(O)₂—N(H)—C(R^{3b})₂—;
Cy³ is substituted or unsubstituted phenylene, pyridylene, pyrimidinylene, substituted or unsubstituted azetidinylene, substituted or unsubstituted pyrrolidinylene, substituted or unsubstituted piperidinylene, or substituted or unsubstituted piperazinylene; and the substitution on Cy³ is selected from 1, 2, or 3 groups independently selected from C₁-C₄ alkyl, hydroxy, CN, C₁-C₄ alkoxy, and halo;
L is a single bond, substituted or unsubstituted —N(H)—, —C(F)₂—O—, or substituted or unsubstituted C₁₋₄ alkylene; the substitution on alkylene is C₁-C₄ alkyl, CN, or halo; and the substitution on-N(H)— is C₁-C₄ alkyl;
i)R¹ is —B-C(R^{6a})═C(R^{6b})—C(O)—R^{6c}, —B—C(R^{6a})═C(R^{6b})—S(O)—R^{6c}, —B—C(R^{6a})═C(R^{6b})—S(O)₂—R^{6c}, —B—C(R^{6a})—C(R^{6b})—P(O)—R^{6a}R^{6b}; or —B—C(R^{6a})═C(R^{6b})—P(O)—OR^{6a}OR^{6b}; B is substituted or unsubstituted C₁₋₄ alkylene; R^{6c} is substituted or unsubstituted alkoxy, substituted or unsubstituted amino, or substituted or unsubstituted heterocycloalkyl, having 1-2 heteroatoms independently selected from nitrogen, oxygen, and sulfur; and Cy⁴ is absent, substituted or unsubstituted azetidinylene, substituted or unsubstituted pyrrolidinylene, substituted or unsubstituted piperidinylene, or substituted or unsubstituted piperazinylene; and the substitution on Cy⁴, and heterocycloalkyl is independently selected from 1, 2, or 3 groups independently selected from C₁-C₄ alkyl, hydroxy, CN, C₁-C₄ alkoxy, and halo; and the substitution on amino is C₁-C₄ alkyl or C₁-C₄ haloalkyl;
ii)R¹ is —C(O)—C(R^{6a})—C(R^{6b})R^{6c}, —S(O)—C(R^{6a})═C(R^{6b})R^{6c}, —S(O)₂—C(R^{6a})═C(R^{6b})R^{6c}, —NR^{3c}—C(O)—C(R^{6a})═C(R^{6b})R^{6c}, —NR^{3c}—S(O)—C(R^{6a})═C(R^{6b})R^{6c}, or —NR^{3c}—S(O)₂—C(R^{6a})—C(R^{6b})R^{6c}; and Cy⁴ is absent;
iii)R¹ is —C(O)—C(R^{6a})—C(R^{6b})R^{6c}, —S(O)—C(R^{6a})═C(R^{6b})R^{6c}, —S(O)₂—C(R^{6a})═C(R^{6b})R^{6c}, —NR^{3c}—C(O)—C(R^{6a})═C(R^{6b})R^{6c}, —NR^{3c}—S(O)—C(R^{6a})═C(R^{6b})R^{6c}, or —NR^{3c}—S(O)₂—C(R^{6a})═C(R^{6b})R^{6c}; Cy⁴ is substituted or unsubstituted azetidinylene, substituted or unsubstituted pyrrolidinylene, substituted or unsubstituted piperidinylene, or substituted or unsubstituted piperazinylene; and R^{6a} is alkyl, substituted with halo, alkoxy, alkylamino, dialkylamino, or heterocycloalkyl;
iv)R¹ is —C(O)—C(R^{6a})═C(R^{6b})R^{6c}, —S(O)—C(R^{6a})—C(R^{6b})R^{6c}, —S(O)₂—C(R^{6a})═C(R^{6b})R^{6c}, —NR^{3c}—C(O)—C(R^{6a})═C(R^{6b})R^{6e}, —NR^{3c}—S(O)—C(R^{6a})═C(R^{6b})R^{6c}, or —NR^{3c}—S(O)₂—C(R^{6a})═C(R^{6b})R^{6c}; Cy⁴ is substituted or unsubstituted azetidinylene, substituted or unsubstituted pyrrolidinylene, substituted or unsubstituted piperidinylene, or substituted or unsubstituted piperazinylene; and the compound is any one of compounds listed in Table 1e;
or
v)R¹ is

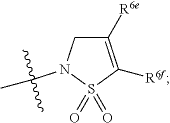

Cy⁴ is absent, substituted or unsubstituted azetidinylene, substituted or unsubstituted pyrrolidinylene, substituted or unsubstituted piperidinylene, or substituted or unsubstituted piperazinylene; each R^{3a}, R^{3b}, and R^{3c} is independently H or substituted or unsubstituted C₁₋₄ alkyl;
each R^{6a} and R^{6b} is independently H, CN, halo, or C₁₋₆ alkyl; or R^{6a} and R^{6b} are joined together to form a bond; R^{6c} is hydroxy, substituted or unsubstituted alkoxy, substituted or unsubstituted amino, substituted or unsubstituted heterocycloalkyl, having 1-2 heteroatoms independently selected from nitrogen, oxygen, and sulfur; each R^{6e} and R^{6f} is independently H, CN, halo, or C₁₋₆ alkyl; and the substitution on heterocycloalkyl is independently selected from 1, 2, or 3 groups independently selected from C₁-C₄ alkyl, hydroxy, CN, C₁-C₄ alkoxy, and halo; and the substitution on amino and alkoxy is C₁-C₄ alkyl; and
R⁷ is a 4-10 membered heterocycloalkyl ring having 1-2 heteroatoms independently selected from nitrogen, oxygen, and sulfur, phenyl, an 8-10 membered bicyclic aryl ring, and a 5-6 membered heteroaryl ring having 1-4 heteroatoms independently selected from nitrogen, oxygen, and sulfur; and the substitution on heterocycloalkyl, phenyl, bicyclic aryl, and heteroaryl is independently selected from 1, 2, or 3 groups independently selected from C₁-C₄ alkyl, hydroxy, CN, C₁-C₄ alkoxy.

In some embodiments, R¹ is —B-C(R^{6a})═C(R^{6b})—C(O)—R^{6c}, —B—C(R^{6a})—C(R^{6b})—S(O)—R^{6c}, or —B—C(R^{6a})—C(R^{6b})—S(O)₂—R^{6c}; B is substituted or unsubstituted C₁₋₄ alkylene; R^{6c} is substituted or unsubstituted alkoxy, substituted or unsubstituted amino, or substituted or unsubstituted heterocycloalkyl, having 1-2 heteroatoms independently selected from nitrogen, oxygen, and sulfur; and Cy⁴ is absent, substituted or unsubstituted azetidinylene, substituted or unsubstituted pyrrolidinylene, substituted or unsubstituted piperidinylene, or substituted or unsubstituted piperazinylene; and the substitution on Cy³, alkylene, alkoxy, is selected from 1, 2, or 3 groups independently selected from C₁-C₄ alkyl, hydroxy, CN, C₁-C₄ alkoxy, and halo; and the substitution on amino is C₁-C₄ alkyl.

In some embodiments, R¹ is —C(O)—C(R^{6a})—C(R^{6b})—C(O)—R^{6c}, —S(O)—C(R^{6a})═C(R^{6b})—C(O)—R^{6c}, or —S(O)₂—C(R^{6a})—C(R^{6b}); and Cy⁴ is absent.

In some embodiments, R¹ is —C(O)—C(R^{6a})═C(R^{6b})R^{6c}, —S(O)—C(R^{6a})—C(R^{6b})R^{6c}, —S(O)₂—C(R^{6a})—C(R^{6b})R^{6c}, —NR^{3c}—C(O)—C(R^{6a})—C(R^{6b})R^{6c}, —NR^{3c}—S(O)—C(R^{6a})—C(R^{6b})R^{6c}, or —NR^{3c}—S(O)₂—C(R^{6a})—C(R^{6b})R^{6c}; and Cy⁴ is absent.

In some embodiments, R¹ is —C(O)—C(R^{6a})—C(R^{6b})R^{6c}, —S(O)—C(R^{6a})—C(R^{6b})R^{6c}, —S(O)₂—C(R^{6a})═C(R^{6b})R^{6c}, —NR^{3c}—C(O)—C(R^{6a})—C(R^{6b})R^{6c}, —NR^{3c}—S(O)—C(R^{6a})═C(R^{6b})R^{6c}, or —NR^{3c}—S(O)₂—C(R^{6a})—C(R^{6b})R^{6c}; Cy⁴ is substituted or unsubstituted heterocycloalkylene; and R^{6a} is alkyl, substituted with halo, substituted or unsubstituted alkoxy, substituted or unsubstituted amino, or substituted or unsubstituted heterocycloalkyl.

In some embodiments, R¹ is —C(O)—C(R^{6a})═C(R^{6b})R^{6c}, —S(O)—C(R^{6a})—C(R^{6b})R^{6c}, —S(O)₂—C(R^{6a})═C(R^{6b})R^{6c}, —NR^{3c}—C(O)—C(R^{6a})═C(R^{6b})R^{6c}, —NR^{3c}—S(O)—C(R^{6a})—C(R^{6b})R^{6c}, or —NR₃₀—S(O)₂—C(R^{6a})—C(R^{6b})R^{6c}; Cy⁴ is substituted or unsubstituted azetidinylene, substituted or unsubstituted pyrrolidinylene, substituted or unsubstituted piperidinylene, or substituted or unsubstituted piperazinylene; and R^{6a} is alkyl, substituted with halo, alkoxy, alkylamino, dialkylamino, or substituted or unsubstituted heterocycloalkyl. In one embodiment, the substitution on heterocycloalkyl is selected from alkyl, hydroxy or halo. In one embodiment, the substitution on azetidinylene, pyrrolidinylene, piperidinylene, pyrrolidinylene; and hetercycloalkyl is selected from 1, 2, or 3 groups independently selected from C₁-C₄ alkyl, hydroxy, CN, C₁-C₄ alkoxy, and halo.

In some embodiments, —C(O)—C(R^{6a})═C(R^{6b})R^{6c}, —S(O)—C(R^{6a})═C(R^{6b})R^{6c}, —S(O)₂—C(R^{6a})—C(R^{6b})R^{6c}, —NR^{3c}—C(O)—C(R^{6a})═C(R^{6b})R^{6c}, —NR^{3c}—S(O)—C(R^{6a})═C(R^{6b})R^{6c}, or —NR^{3c}—S(O)₂—C(R^{6a})═C(R^{6b})R^{6c}; Cy⁴ is substituted or unsubstituted heterocycloalkylene.

In some embodiments, —C(O)—C(R^{6a})═C(R^{6b})R^{6c}, —S(O)—C(R^{6a})═C(R^{6b})R^{6c}, —S(O)₂—C(R^{6a})—C(R^{6b})R^{6c}, —NR^{3c}—C(O)—C(R^{6a})═C(R^{6b})R^{6c}, —NR^{3c}—S(O)—C(R^{6a})—C(R^{6b})R^{6c}, or —NR^{3c}—S(O)₂—C(R^{6a})═C(R^{6b})R^{6c}; Cy⁴ is substituted or unsubstituted azetidinylene, substituted or unsubstituted pyrrolidinylene, substituted or unsubstituted piperidinylene, or substituted or unsubstituted piperazinylene. In some embodiments, the compound is any one of compounds listed in Table 1e. In some embodiments, the substitution is independently selected from 1, 2, or 3 of C₁-C₄ alkyl, amino, alkylamino, dialkylamino, hydroxy, C₁-C₄ hydroxyalkyl, CN, C₁-C₄ alkoxy, and halo.

In some embodiments, R¹ is

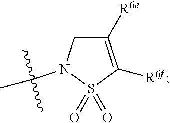

and Cy⁴ is absent, substituted or unsubstituted azetidinylene, substituted or unsubstituted pyrrolidinylene, substituted or unsubstituted piperidinylene, or substituted or unsubstituted piperazinylene; and the substitution on each of Cy³, alkylene, alkoxy, is selected from 1, 2, or 3 groups independently selected from C₁-C₄ alkyl, hydroxy, CN, C₁-C₄ alkoxy, and halo; and the substitution on amino is C₁-C₄ alkyl. In some embodiments, the substitution on Cy³, alkylene, is selected from 1, 2, or 3 groups independently selected from C₁-C₄ alkyl, hydroxy, CN, C₁-C₄ alkoxy, and halo; and the substitution on amino is C₁-C₄ alkyl.

In another aspect, provided herein is a compound according to Formula (L-I); and wherein the compound is according to formula LXIa, LXIb, LXII, LXIII, LXIV, LXV, or LXVI; and wherein the formula LXI, LXII, LXIII, LXIV, LXV, and LXVI are as described herein.

In particular embodiments, the compound is according to formula (I):

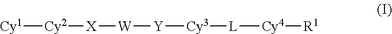

or a stereoisomer thereof; or a pharmaceutically acceptable salt thereof,
wherein:
Cy¹ is substituted or unsubstituted

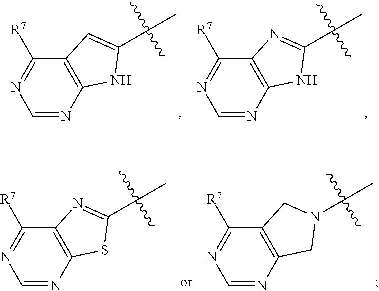

Cy² is substituted or unsubstituted phenylene;
X is —NR^{3a}—, —C(R^{3b})₂—, or —O—;
Y is a single bond, —NR^{3a}—, —C(R^{3b})₂—, or —O—;
W is —C(O)—, —S(O)—, or —S(O)₂—;
Cy³ is substituted or unsubstituted phenylene, or pyridylene;
L is a single bond, or —CH₂—;
Cy⁴ is absent, substituted or unsubstituted azetidinylene, substituted or unsubstituted pyrrolidinylene, substituted or unsubstituted piperidinylene, or substituted or unsubstituted piperazinylene;
R¹ is —C(O)—B—C(R^{6a})═C(R^{6b})—C(O)—R^{6c}, —S(O)—B—C(R^{6a})═C(R^{6b})—C(O)—R^{6c}, —S(O)₂—B—C(R^{6a})—C(R^{6b})—C(O)—R^{6c}, —B—C(R^{6a}) ═C(R^{6b})—C(O)—R^{6c}, —B—C(R^{6a})—C(R^{6b})—S(O)—R^{6c}; —B-C(R^{6a})—C(R^{6b})—S(O)₂—R^{6c}; B is substituted or unsubstituted C₁₋₄ alkylene;
each R^{3a}, R^{3b}, and R^{3c} is independently H or substituted or unsubstituted C₁₋₄ alkyl;
each R^{6a} and R^{6b} is independently H, CN, halo, or C₁₋₆ alkyl; or R^{6a} and R^{6b} are joined together to form a bond; R^{6c} is substituted or unsubstituted alkoxy, substituted or unsubstituted amino, substituted or unsubstituted heterocycloalkyl, having 1-2 heteroatoms independently selected from nitrogen, oxygen, and sulfur;
R⁷ is H, an optionally substituted group selected from alkyl, a 4-10 membered heterocycloalkyl ring having 1-2 heteroatoms independently selected from nitrogen, oxygen, and sulfur, phenyl, an 8-10 membered bicyclic aryl ring, and a 5-6 membered heteroaryl ring having 1-4 heteroatoms independently selected from nitrogen, oxygen, and sulfur.

In some embodiments, each R^{3a}, R^{3b}, and R^{3c} is independently H or substituted or unsubstituted C₁₋₄ alkyl; and the substitution on alkyl is selected from 1, 2, or 3 groups selected from halo, alkoxy, or CN. In some embodiments, each R^{3a}, R^{3b}, and R^{3c} is independently H, CH₂Cl, CH₂F, CHF₂, CF₃, OMe, or CN.

In certain embodiments, with respect to any of the formulas described herein, Cy¹ is substituted or unsubstituted

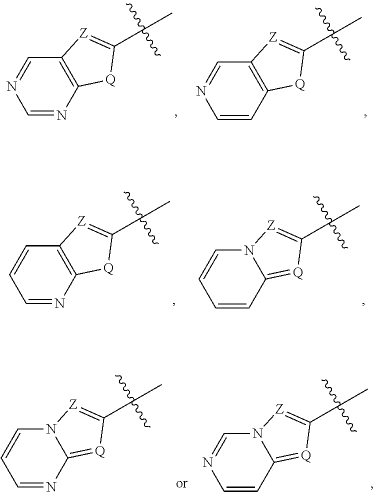

wherein Q is N, —N(H)—, —O—, or —S—; Z is —CR^{5a}═ or —N═; and R^{5a} is H, C₁₋₆ alkyl, C₁₋₆ haloalkyl, halo, or CN;
and Cy², Cy³, Cy⁴, L, W, X, Y, and R¹ are as described herein.

In certain embodiments, with respect to any of the formulas described herein, Cy¹ is substituted or unsubstituted

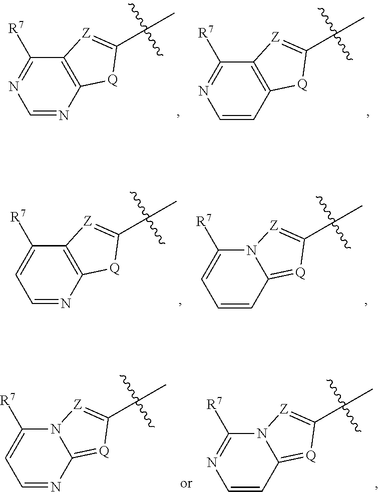

wherein Q is N, —N(H)—, —O—, or —S—; Z is —CR^{5a}═ or —N═; and R^{5a} is H, C₁₋₆ alkyl, C₁₋₆ haloalkyl, halo, or CN; and Cy², Cy³, Cy⁴, L, W, X, Y, and R¹ are as described herein.

In certain embodiments, the compound is according to Formula (LX-I):

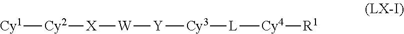

or a pharmaceutically acceptable salt thereof,
wherein:
Cy¹, Cy², X, W, Y, Cy³, L, Cy⁴ and R¹ are as described herein.

In one embodiment, R^{3a} is H or substituted or unsubstituted C₁₋₄ alkyl; and each of R^{3b}, and R^{3c} is independently H, OH, substituted or unsubstituted C₁₋₄ alkoxy.

In one embodiment, Cy⁴ is substituted or unsubstituted heterocycloalkyl, and the heterocycloalkyl includes bicyclic, fused and bridged heterocycloalkyls. In one embodiment, Cy⁴ is a bridged heterocycloalkyl.

In another aspect, provided herein is a compound according to Formula (L-I) having the structure:

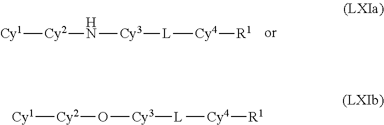

or a stereoisomer thereof; or a pharmaceutically acceptable salt thereof, wherein Cy¹, Cy², Cy³, Cy⁴, L, and R¹ are as described herein.

In another aspect, provided herein is a compound according to Formula (L-I) having the structure:

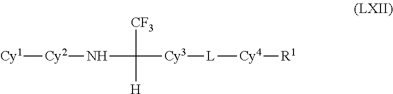

or a pharmaceutically acceptable salt thereof, wherein Cy¹, Cy², Cy³, Cy⁴, L, and R¹ are as described herein.

In another aspect, provided herein is a compound according to Formula (L-I) having the structure:

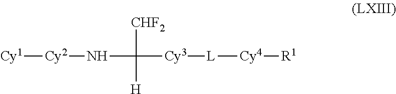

or a pharmaceutically acceptable salt thereof, wherein Cy¹, Cy², Cy³, Cy⁴, L, and R¹ are as described herein.

In another aspect, provided herein is a compound according to Formula (L-I) having the structure:

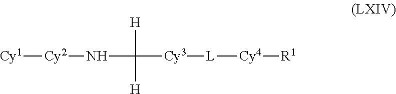

or a stereoisomer thereof; or a pharmaceutically acceptable salt thereof, wherein Cy¹, Cy², Cy³, Cy⁴, L, and R¹ are as described herein.

In another aspect, provided herein is a compound according to Formula (L-I) having the structure:

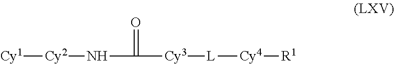

or a stereoisomer thereof; or a pharmaceutically acceptable salt thereof, wherein Cy¹, Cy², Cy³, Cy⁴, L, and R¹ are as described herein.

In another aspect, provided herein is a compound according to Formula (L-I) having the structure:

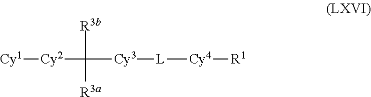

or a stereoisomer thereof; or a pharmaceutically acceptable salt thereof, wherein Cy¹, Cy², Cy³, Cy⁴, L, R¹, and R^{3a} are as described herein; and R^{3b} is OH, or substituted or unsubstituted C₁₋₄ alkoxy.

In a particular embodiment, the compound is according to formula LXIa, LXIb, LXII, LXIII, LXIV, LXV, or LXVI. In another particular embodiment, the compound is according to formula LXV, and R^{6a} substituted alkyl. In a more particular embodiment, the compound is according to formula LXV, and R^{6a} methyl substituted with amino, alkylamino, dialkylamino, or heterocycloalkyl.

In certain particular embodiments, Cy¹ is

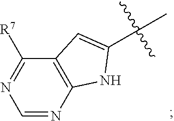

and R⁷ is as described herein.

In some embodiments, Cy¹ is substituted or unsubstituted

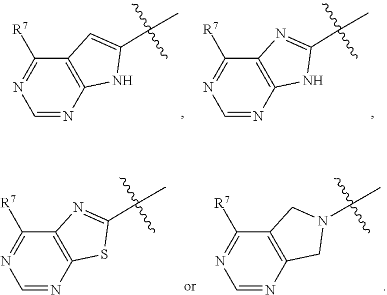

In some embodiments, Cy¹ is substituted or unsubstituted

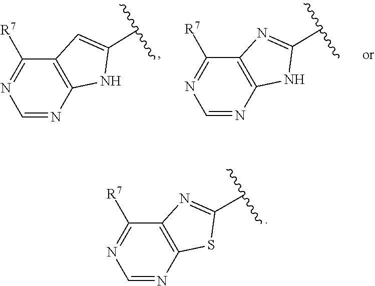

In some embodiments, Cy¹ is substituted or unsubstituted

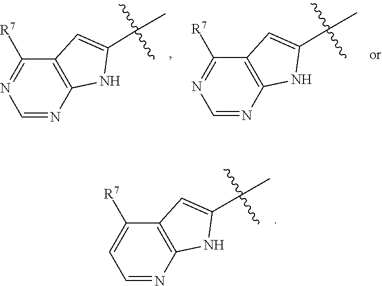

In some embodiments, Cy¹ is substituted or unsubstituted

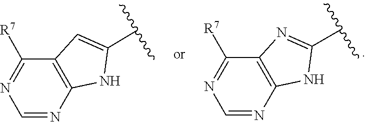

In some embodiments, Cy¹ is substituted or unsubstituted

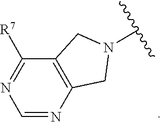

In some embodiments, Cy¹ is substituted or unsubstituted

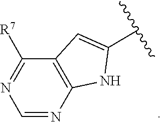

In some embodiments, Cy¹ is substituted or unsubstituted

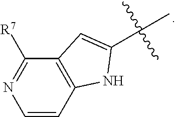

In some embodiments, Cy¹ is substituted or unsubstituted

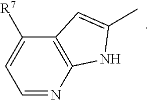

In some embodiments, Cy¹ is substituted or unsubstituted

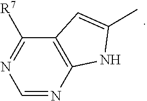

In some embodiments, Cy¹ is substituted and the substitution is C₁-C₄ alkyl, CN, or halo. In a particular embodiment, the substitution is C₁-C₄ alkyl. In a more particular embodiment, Cy¹ is unsubstituted.

In some embodiments, the compound is according to formula (II):

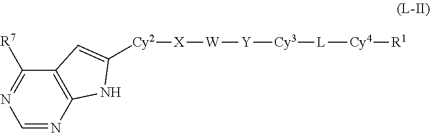

or a stereoisomer thereof; or a pharmaceutically acceptable salt thereof; wherein R¹, R⁷, Cy², Cy³, Cy⁴, X, W, and Y are as described for formula (L-I).

In some embodiments, the compound is according to formula (II):

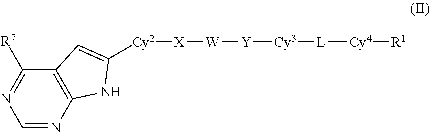

or a stereoisomer thereof; or a pharmaceutically acceptable salt thereof,
wherein:
Cy² is substituted or unsubstituted

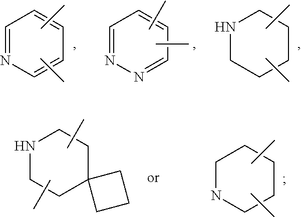

X is —NR^{3a}—, —C(R^{3b})₂—, or —O—;
W is —C(R^{3b})₂—, —C(O)—, —S(O)—, or —S(O)₂—;
Y is absent, —NR^{3a}—, —C(R^{3b})₂—, or —O—;
Cy³ is substituted or unsubstituted phenylene, pyridylene, pyrimidinylene, or piperidinylene;
L is a single bond, substituted or unsubstituted —N(H)—, or substituted or unsubstituted C₁₋₄ alkylene;
Cy⁴ is absent, substituted or unsubstituted azetidinylene, substituted or unsubstituted pyrrolidinylene, substituted or unsubstituted piperidinylene, or substituted or unsubstituted piperazinylene;
R¹ is —C(O)—B—C(R^{6a})—C(R^{6b})—C(O)—R^{6c}, —S(O)—B—C(R^{6a})═C(R^{6b})—C(O)—R^{6c}, —S(O)₂—B—C(R^{6a})═C(R^{6b})—C(O)—R^{6c}, —B—C(R^{6a})=C(R^{6b})—C(O)—R^{6c}, —B—C(R^{6a})═C(R^{6b})—S(O)—R^{6c}; —B-C(R^{6a}) ═C(R^{6b})—S(O)₂—R^{6c}; B is substituted or unsubstituted C₁₋₄ alkylene;
each R^{3a}, R^{3b}, and R^{3c} is independently H or substituted or unsubstituted C₁₋₄ alkyl;
each R^{6a} and R^{6b} is independently H, CN, halo, or C₁₋₆ alkyl; or R^{6a} and R^{6b} are joined together to form a bond; R^{6c} is substituted or unsubstituted alkoxy, substituted or unsubstituted amino, substituted or unsubstituted heterocycloalkyl, having 1-2 heteroatoms independently selected from nitrogen, oxygen, and sulfur;
R⁷ is H, an optionally substituted group selected from alkyl, a 4-10 membered heterocycloalkyl ring having 1-2 heteroatoms independently selected from nitrogen, oxygen, and sulfur, phenyl, an 8-10 membered bicyclic aryl ring, and a 5-6 membered heteroaryl ring having 1-4 heteroatoms independently selected from nitrogen, oxygen, and sulfur.

In some embodiments, Cy² is substituted or unsubstituted

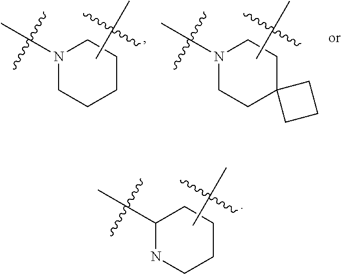

In some embodiments, Cy² is substituted or unsubstituted

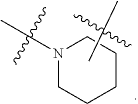

In some embodiments, Cy² is substituted or unsubstituted

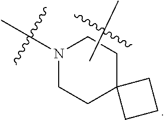

In some embodiments, Cy² is substituted or unsubstituted

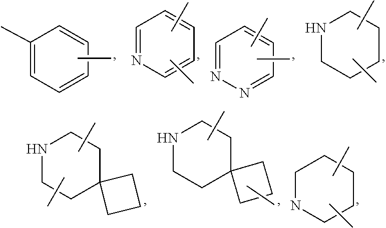

In some embodiments, Cy² is substituted or unsubstituted

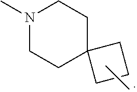

In some embodiments, Cy² is substituted or unsubstituted phenylene, pyridylene, or pyrazinyl.

In some embodiments, Cy² is substituted or unsubstituted phenylene.

In some embodiments, Cy² is substituted or unsubstituted

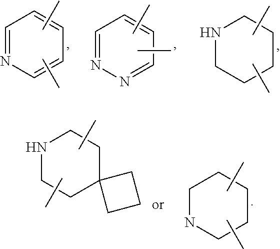

When a bond is depicted between the atoms of one or more rings, it can be bonded to any atom of the ring with a free valence, for instance substituting for a hydrogen atom. When there are two or more rings, fused, spiro, bridging, or otherwise, the bond can be bonded to any atom of any ring with a free valence, for instance substituting for a hydrogen atom, unless specified otherwise.

In some embodiments, Cy² is substituted or unsubstituted

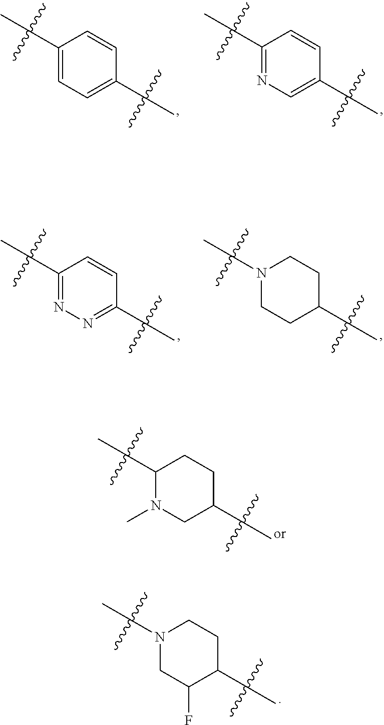

In some embodiments, Cy² is substituted or unsubstituted

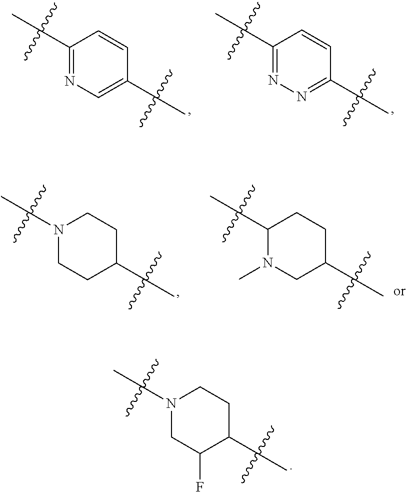

In some embodiments, Cy² is substituted or unsubstituted

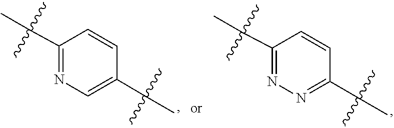 [SMILES: *c1ccc(*)nc1 ; *c1ccc(*)nn1]

In some embodiments, Cy² is substituted or unsubstituted

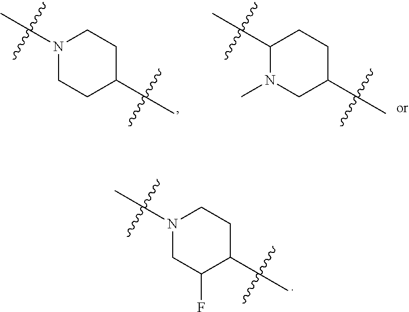

In some embodiments, Cy² is substituted or unsubstituted

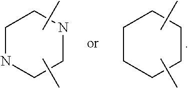

In some embodiments, the substitution on Cy² is independently selected from 1, 2, or 3 of C₁-C₄ alkyl, hydroxy, CN, C₁-C₄ alkoxy, and halo. In particular embodiments, Cy² is substituted or unsubstituted phenylene, or substituted or unsubstituted pyridinyl; and the substitution is independently selected from 1, 2, or 3 of C₁-C₄ alkyl, hydroxy, CN, C₁-C₄ alkoxy, and halo.

In some embodiments, Cy² is substituted or unsubstituted phenylene,

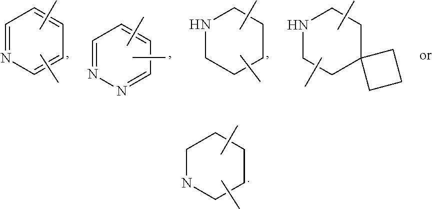

In some embodiments, X is N(H) or C(H)₂. In some embodiments, X is C(H)₂. In some embodiments, X is N(H). In some embodiments, W is C(O), C(H)₂, or C(H)(CF₃). In some embodiments, W is C(O). In some embodiments, W is C(H)₂. In some embodiments, W is C(H)(CF₃). In some embodiments, Y is absent, N(H), or C(H)₂. In some embodiments, Y is C(H) 2.

In some embodiments, Y is N(H). In some embodiments, Y is absent.

In some embodiments, —X—W—Y—is —N(H)—C(O)—N(H)—, —N(H)—C(O)—CH₂—, —CH₂—C(O)—N(H)—, —N(H)—S(O)—N(H)—, —N(H)—S(O)—CH₂—, —CH₂—S(O)—N(H)—, —N(H)—S(O)₂—N(H)—, —N(H)—S(O)₂—CH₂—, —CH₂—S(O)₂—N(H)—, —N(H)—C(O)—, —N(H)—C(H)₂—, —C(H)₂—N(H)₂—, or —N(H)—C(H)(CF₃)—C(H)₂—.

In some embodiments, —X—W—Y—is —N(H)—C(O)—N(H)—. In some embodiments, —X—W—Y—is —N(H)—C(O)—CH₂—. In some embodiments, —X—W—Y—is —N(H)—C(O)—. In some embodiments, —X—W—Y—is —N(H)—C(H)(CF₃)—C(H)₂—. In some embodiments, —X—W—Y—is —N(H)—, or —S(O)₂—N(H)—C(R^{3b})₂—.

In some embodiments, —X—W—Y—is —N(H)—. In some embodiments, —X—W—Y—is —S(O)₂—N(H)—C(H)₂—. In some embodiments, —X—W—Y—is —N(H)—C(O)—, —C(O)—N(H)—, —S(O)₂—N(H)—, —N(H)—C(O)—N(H)—, —N(H)—S(O)₂—CH₂—, —N(H)—S(O)₂—C(Me)H—, —N(H)—S(O)₂—C(Me)₂-, —N(H)—C(H)₂—, —N(Me)—S(O)₂—CH₂—, —N(Me)—C(O)—, —N(H)—C(O)—CH₂—, or —N(H)—C(O)—C(Me)₂—. In some embodiments, —X—W—Y—is —N(H)—C(CF₃)H—, In some embodiments, —X—W—Y—is —N(H)—C(CHF₂) H—. In some embodiments, —X—W—Y—is —O—CH₂—, or —O—C(Me)H—.

In certain embodiments, Cy¹-Cy²—X—W—Y-Cy³-L-Cy⁴-R¹ is

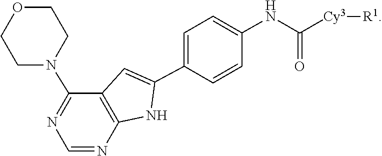

In certain embodiments, the compound is according to Formula (LX-IIIa) or (LX-IIIb):

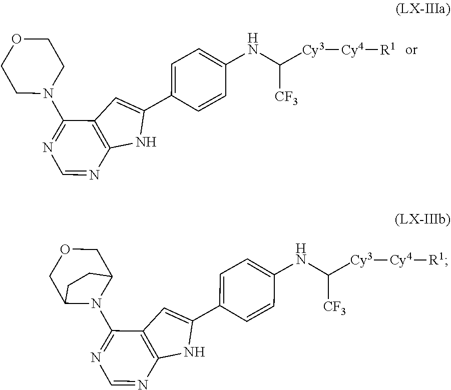

and wherein Cy³, Cy⁴, and R¹ is as described herein.

In certain embodiments, the compound is according to Formula (LX-IVa) or (LX-IVb):

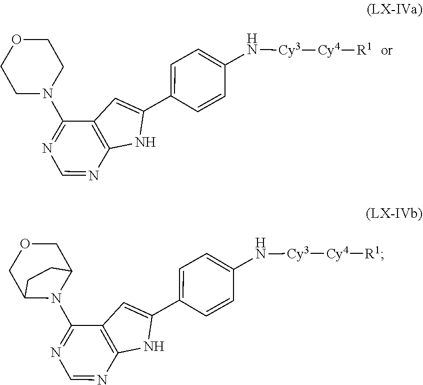

and wherein Cy³, Cy⁴, and R¹ is as described herein.

In certain embodiments, Cy³ is as described herein. In certain embodiments, Cy³ is substituted or unsubstituted heterocycloalkyl. In certain embodiments, Cy³ is substituted or unsubstituted azetidinyl, pyrrolidinyl, piperidinyl, or piperazinyl. In certain embodiments, Cy³ is substituted or unsubstituted piperidinyl. In certain embodiments, Cy³ is absent.

In certain embodiments, Cy³ is absent; and Cy⁴ is substituted or unsubstituted azetidinyl, pyrrolidinyl, piperidinyl, or piperazinyl. In certain embodiments, Cy³ is absent; and Cy⁴ is substituted or unsubstituted piperidinyl. In certain embodiments, Cy³ is absent; and Cy⁴ is substituted or unsubstituted piperidin-3-yl. In certain embodiments, Cy³ is absent; and Cy⁴ is substituted or unsubstituted piperidin-4-yl.

In certain embodiments, Cy³ is azetidinyl; and Cy⁴ is substituted or unsubstituted azetidinyl, pyrrolidinyl, piperidinyl, or piperazinyl. In certain embodiments, Cy³ is azetidinyl; and Cy⁴ is substituted or unsubstituted piperidinyl. In certain embodiments, Cy³ is azetidinyl; and Cy⁴ is substituted or unsubstituted piperidin-3-yl. In certain embodiments, Cy³ is azetidinyl; and Cy⁴ is substituted or unsubstituted piperidin-4-yl.

In certain embodiments, Cy³ is pyrrolidinyl; and Cy⁴ is substituted or unsubstituted azetidinyl, pyrrolidinyl, piperidinyl, or piperazinyl. In certain embodiments, Cy³ is pyrrolidinyl; and Cy⁴ is substituted or unsubstituted piperidinyl. In certain embodiments, Cy³ is pyrrolidinyl; and Cy⁴ is substituted or unsubstituted piperidin-3-yl. In certain embodiments, Cy³ is pyrrolidinyl; and Cy⁴ is substituted or unsubstituted piperidin-4-yl.

In certain embodiments, Cy³ is piperidinyl; and Cy⁴ is substituted or unsubstituted azetidinyl, pyrrolidinyl, piperidinyl, or piperazinyl. In certain embodiments, Cy³ is piperidinyl; and Cy⁴ is substituted or unsubstituted piperidinyl. In certain embodiments, Cy³ is piperidinyl; and Cy⁴ is substituted or unsubstituted piperidin-3-yl. In certain embodiments, Cy³ is piperidinyl; and Cy⁴ is substituted or unsubstituted piperidin-4-yl. In certain embodiments, Cy³ is piperidinyl; and Cy⁴ is substituted or unsubstituted azetidinyl. In certain embodiments, Cy³ is piperidinyl; and Cy⁴ is substituted or unsubstituted pyrrolidinyl.

In certain embodiments, Cy³ is substituted or unsubstituted pyridyl, pyrimidinyl, or pyridazine; and Cy⁴ is substituted or unsubstituted azetidinyl, pyrrolidinyl, piperidinyl, or piperazinyl. In certain embodiments, Cy³ is substituted or unsubstituted pyridyl, or pyrimidinyl, and Cy⁴ is substituted or unsubstituted piperidinyl. In certain embodiments, Cy³ is substituted or unsubstituted pyridyl, or pyrimidinyl, and Cy⁴ is substituted or unsubstituted piperazinyl.

In some embodiments, Cy³ is substituted or unsubstituted phenyl, pyridyl, pyrimidinyl, pyrrolidinyl, piperidinyl, or piperazinyl; and the substitution is independently selected from 1, 2, or 3 of C₁-C₄ alkyl, amino, alkylamino, dialkylamino, hydroxy, C₁-C₄ hydroxyalkyl, CN, C₁-C₄ alkoxy, and halo.

In some embodiments, Cy³ is substituted or unsubstituted phenyl, pyridyl, or pyrimidinyl.

In some embodiments, Cy³ is substituted or unsubstituted piperidinyl. In some embodiments, Cy³ is substituted or unsubstituted pyridyl, or pyrimidinyl, or piperidinyl. In some embodiments, Cy³ is substituted or unsubstituted phenyl, or pyridyl. In some embodiments, Cy³ is substituted or unsubstituted phenyl. In some embodiments, Cy³ is substituted or unsubstituted

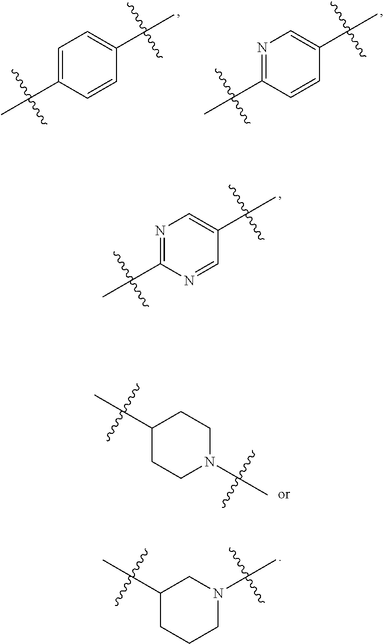 [SMILES: *c1ccc(*)cc1 ; *c1ccc(*)nc1 ; *c1cnc(*)nc1 ; *C1CCN(*)CC1 ; *C1CCCN(*)C1]

In some embodiments, Cy³ is substituted or unsubstituted

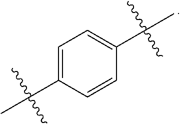 [SMILES: *c1ccc(*)cc1]

In some embodiments, Cy³ is substituted or unsubstituted

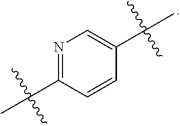 [SMILES: *c1ccc(*)nc1]

In some embodiments, Cy³ is substituted or unsubstituted

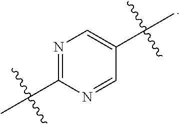 [SMILES: *c1cnc(*)nc1]

In some embodiments, Cy³ is substituted or unsubstituted

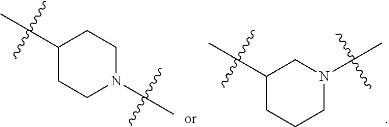 [SMILES: *C1CCN(*)CC1 ; *C1CCCN(*)C1]

In certain embodiments, Cy³ is substituted or unsubstituted

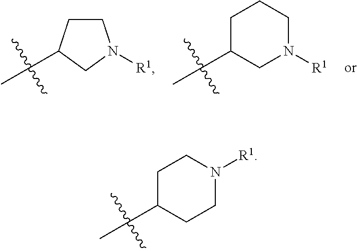

In certain embodiments, Cy³ is substituted or unsubstituted

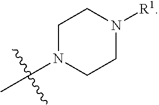

In certain embodiments, Cy³ is substituted or unsubstituted

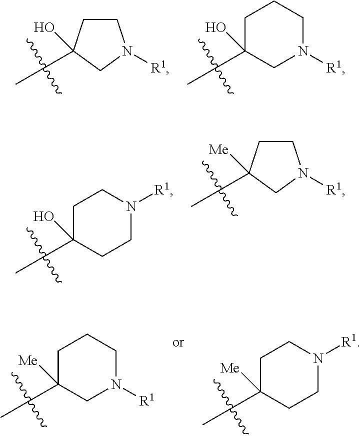

In certain embodiments, Cy³ is substituted or unsubstituted spiro heterocycloalkyl.

In certain embodiments, Cy³ is substituted or unsubstituted

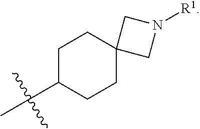

In certain embodiments, the substitution is C₁-C₄ alkyl, or hydroxy.

In certain embodiments, Cy³ is

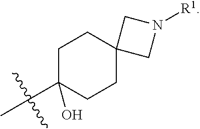

In certain particular embodiments, Cy² is phenyl.

In certain particular embodiments, Cy³ is pyridyl or pyrimidinyl. In certain particular embodiments, Cy³ is 2-pyridyl or 5-pyrimidinyl. In certain particular embodiments, Cy³ is piperidinyl. In certain particular embodiments, Cy³ is 4-piperidinyl. In certain particular embodiments, Cy³ is 1-piperidinyl.

In certain particular embodiments, L is a single bond. In certain particular embodiments, L is —CH₂—.

In certain embodiments, Cy³ is substituted or unsubstituted azetidinylene, substituted or unsubstituted pyrrolidinylene, substituted or unsubstituted piperidinylene, or substituted or unsubstituted piperazinylene.

In certain embodiments, Cy³ is pyrrolidinyl, or piperidinyl, unsubstituted or substituted with alkyl, or —OH.

In certain embodiments, Cy³ is pyrrolidinyl, or piperidinyl, unsubstituted or substituted with Me, Et, i-Pr, or —OH.

In some embodiments, L is a single bond, or substituted or unsubstituted C₁₋₄ alkylenyl. In some embodiments, L is substituted or unsubstituted —N(H)—. In some embodiments, L is a single bond. In some embodiments, L is substituted or unsubstituted C₁₋₄ alkylenyl. In some embodiments, L is —CH₂—CH₂—. In some embodiments, L is —CH₂—. In some embodiments, L is —C(F₂)—O—. In some embodiments, L is —N(H)—.

In some particular embodiments, Cy¹-Cy²—X—W—Y-Cy³-L-Cy⁴- is

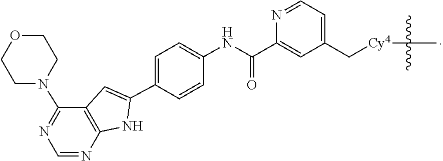

In some particular embodiments, Cy¹-Cy²—X—W—Y-Cy³-L-Cy⁴- is

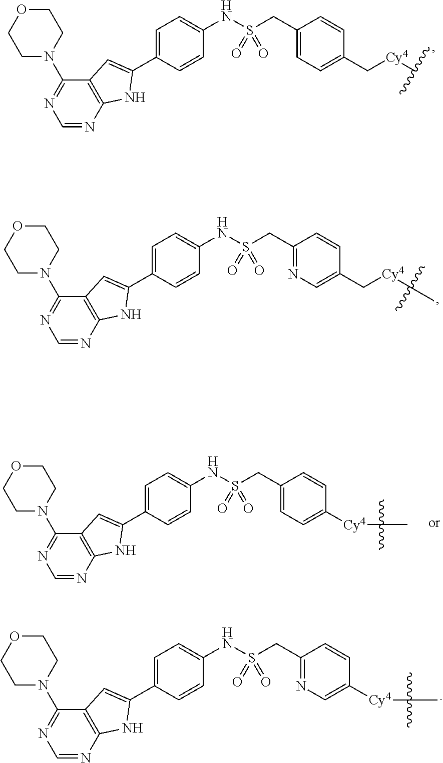

In certain embodiments, Cy⁴ is as described herein. In certain embodiments, Cy⁴ is substituted or unsubstituted heterocycloalkyl. In certain embodiments, Cy⁴ is substituted or unsubstituted azetidinyl, pyrrolidinyl, piperidinyl, or piperazinyl. In certain embodiments, Cy⁴ is substituted or unsubstituted piperidinyl.

In some embodiments, Cy⁴ is substituted or unsubstituted azetidinylene, substituted or unsubstituted pyrrolidinylene, substituted or unsubstituted piperidinylene, or substituted or unsubstituted piperazinylene. In some embodiments, Cy⁴ is unsubstituted azetidinylene, unsubstituted pyrrolidinylene, unsubstituted piperidinylene, or unsubstituted piperazinylene.

In some embodiments, Cy⁴ is azetidinylene, pyrrolidinylene, piperidinylene, or piperazinylene, each substituted with one or more alkylene, halo, or alkoxy. In some embodiments, Cy⁴ is azetidinylene, pyrrolidinylene, piperidinylene, or piperazinylene, each substituted with Me, Et, F, (F)₂, or Cl. In some embodiments, Cy⁴ is absent.

In some embodiments, Cy⁴ is

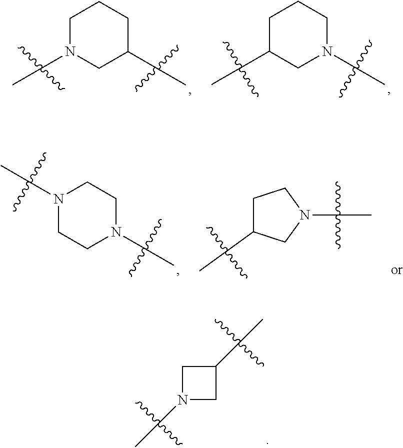 [SMILES: *C1CCCN(*)C1 ; *C1CCCN(*)C1 ; *N1CCN(*)CC1 ; *C1CCN(*)C1 ; *C1CN(*)C1]

In some embodiments, Cy⁴ is

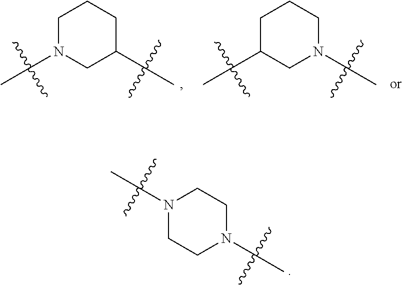 [SMILES: *C1CCCN(*)C1 ; *C1CCCN(*)C1 ; *N1CCN(*)CC1]

In some embodiments, Cy⁴ is

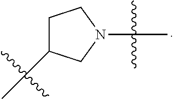 [SMILES: *C1CCN(*)C1]

In some embodiments, Cy⁴ is

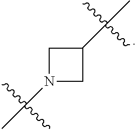 [SMILES: *C1CN(*)C1]

In some embodiments, Cy⁴ is

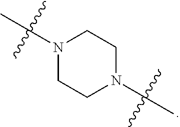 [SMILES: *N1CCN(*)CC1]

In some particular embodiments, Cy⁴ is

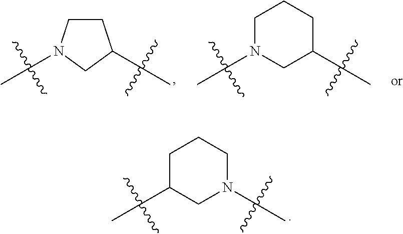 [SMILES: *C1CCN(*)C1 ; *C1CCCN(*)C1 ; *C1CCCN(*)C1]

In some particular embodiments, Cy⁴-R¹ is:

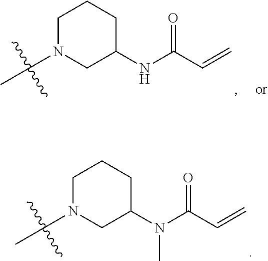 [SMILES: *N1CCCC(NC(=O)C=C)C1 ; *N1CCCC(N(C)C(=O)C=C)C1]

In some particular embodiments, Cy⁴ is substituted or unsubstituted

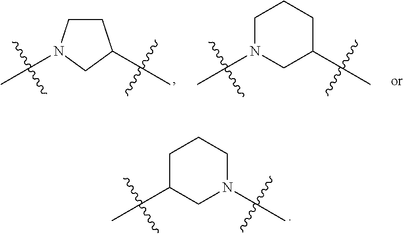 [SMILES: *C1CCN(*)C1 ; *C1CCCN(*)C1 ; *C1CCCN(*)C1]

In some particular embodiments, Cy⁴ is as described herein and the substation is Me, Et, i-Pr, halo or dihalo. In some particular embodiments, Cy⁴ is as described herein and the substation is Me, Et, i-Pr, Cl, F, or difluoro.

In certain embodiments, Cy⁴ is substituted or unsubstituted azetidinylene, substituted or unsubstituted pyrrolidinylene, substituted or unsubstituted piperidinylene, or substituted or unsubstituted piperazinylene; and the substitution is independently selected from 1, 2, or 3 of C₁-C₄ alkyl, amino, alkylamino, dialkylamino, hydroxy, C₁-C₄ hydroxyalkyl, CN, C₁-C₄ alkoxy, and halo. In certain embodiments, Cy⁴ is unsubstituted azetidinylene, unsubstituted pyrrolidinylene, unsubstituted piperidinylene, or unsubstituted piperazinylene. In certain embodiments, Cy⁴ is azetidinylene, pyrrolidinylene, piperidinylene, or piperazinylene, each substituted with one or more alkyl, halo, or alkoxy. In certain embodiments, Cy⁴ is azetidinylene, pyrrolidinylene, piperidinylene, or piperazinylene, each substituted with Me, Et, F, (F)₂, or Cl.

In some particular embodiments, Cy⁴ is

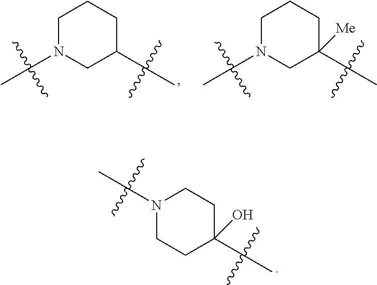 [SMILES: *C1CCCN(*)C1 ; *N1CCCC(*)(C)C1 ; *N1CCC(*)(O)CC1]

In some particular embodiments, Cy⁴ is

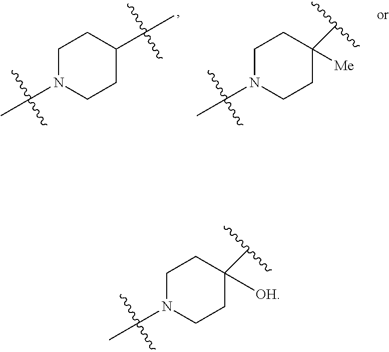 [SMILES: "or" ; *C1CCN(*)CC1 ; *N1CCC(*)(C)CC1 ; *N1CCC(*)(O)CC1]

In some particular embodiments, Cy⁴-R¹ is

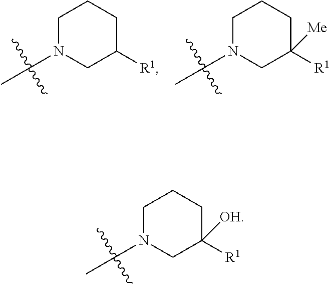

In some particular embodiments, Cy⁴ is

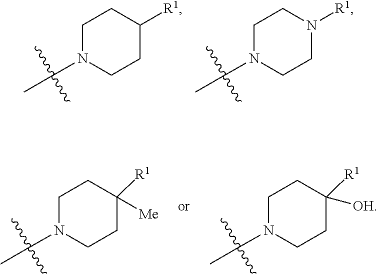

In some particular embodiments, Cy⁴ is

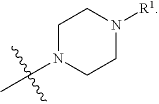

In certain embodiments, Cy⁴ is substituted or unsubstituted heterocycloalkyl; and the substitution is independently selected from 1, 2, or 3 of C₁-C₄ alkyl, amino, alkylamino, dialkylamino, hydroxy, C₁-C₄ hydroxyalkyl, CN, C₁-C₄ alkoxy, and halo.

In certain embodiments, Cy⁴ is substituted or unsubstituted azetidinylene, substituted or unsubstituted pyrrolidinylene, substituted or unsubstituted piperidinylene, substituted or unsubstituted piperazinylene, substituted or unsubstituted diazabicycloheptanyl, or substituted or unsubstituted diazabicyclooctanyl; and the substitution is independently selected from 1, 2, or 3 of C₁-C₄ alkyl, amino, alkylamino, dialkylamino, hydroxy, C₁-C₄ hydroxyalkyl, CN, C₁-C₄ alkoxy, and halo.

In certain embodiments, Cy⁴ is unsubstituted azetidinylene, unsubstituted pyrrolidinylene, unsubstituted piperidinylene, or unsubstituted piperazinylene.

In certain embodiments, Cy⁴ is azetidinylene, pyrrolidinylene, piperidinylene, or piperazinylene, each substituted with one or more alkyl, halo, or alkoxy.

In certain embodiments, Cy⁴ is azetidinylene, pyrrolidinylene, piperidinylene, or piperazinylene, each substituted with Me, Et, F, (F)₂, or Cl.

In certain embodiments, Cy⁴ is diazabicycloheptanyl or diazabicyclooctanyl, each unsubstituted or substituted with one or more alkyl, halo, or alkoxy.

In certain embodiments, Cy⁴ is

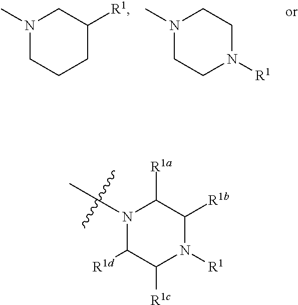

and wherein at least one pair of R^{1a} and R^{1c}, R^{1a} and R^{1d}, R^{1b} and R^{1c}, or R^{1b} and R^{1d} is joined together to form heterocycloalkyl, and the rest of R^{1a}, R^{1b}, R^{1c}, and R^{1d} are each H.

In certain embodiments, Cy⁴ is

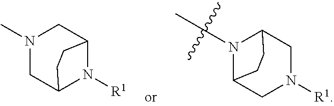

In some embodiments, —X—W—Y—is —NH—C(O), —NH—, or —NH—CH(CF₃)—.

In some embodiments, Cy³ is substituted or unsubstituted phenylene, or pyridylene.

In some embodiments, L is —CH₂—.

In some embodiments, Cy¹ is substituted or unsubstituted

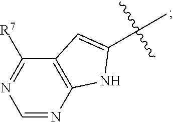

—X—W—Y—is —NH—C(O); Cy³ is substituted or unsubstituted phenylene, or pyridylene; and L is —CH₂—; and Cy² is substituted or unsubstituted phenylene,

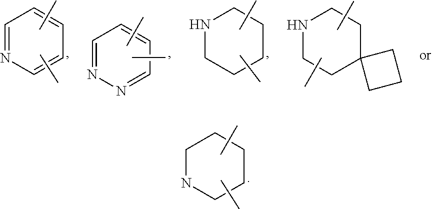

In some particular embodiments, R⁷-Cy¹-Cy²—X—W—Y-Cy³ is:

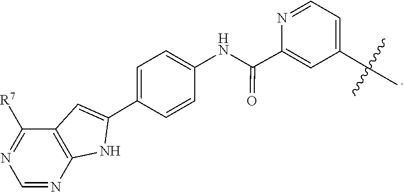

In some particular embodiments, R⁷-Cy¹-Cy²—X—W—Y-Cy³ is:

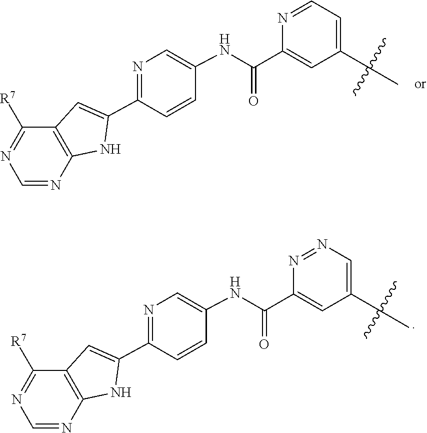

In some particular embodiments, R⁷-Cy¹-Cy²—X—W—Y-Cy³ is:

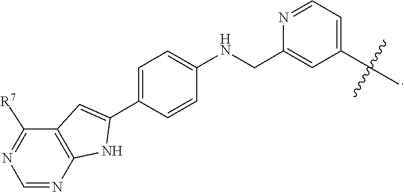

In some particular embodiments, R⁷-Cy¹-Cy²—X—W—Y-Cy³ is:

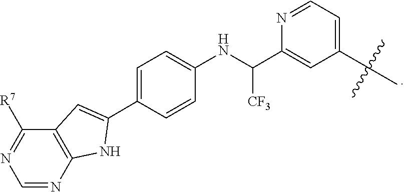

In some particular embodiments, R⁷-Cy¹-Cy²—X—W—Y-Cy³ is:

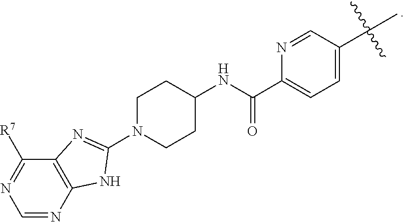

In some particular embodiments, R⁷-Cy¹-Cy²—X—W—Y-Cy³ is:

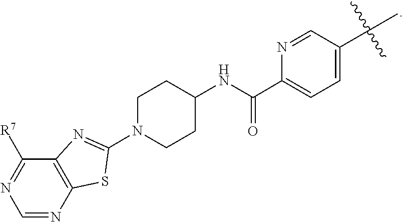

In some particular embodiments, R⁷-Cy¹-Cy²—X—W—Y-Cy³ is:

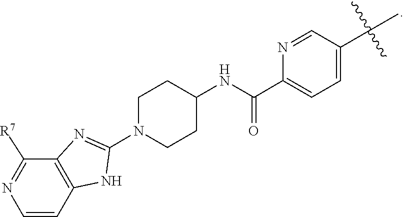

In some particular embodiments, R⁷-Cy¹-Cy²—X—W—Y-Cy³ is:

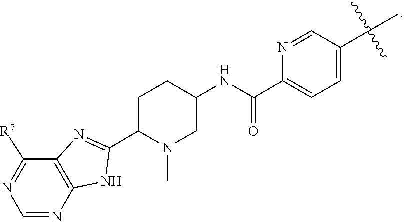

In some particular embodiments, R⁷-Cy¹-Cy²—X—W—Y-Cy³ is:

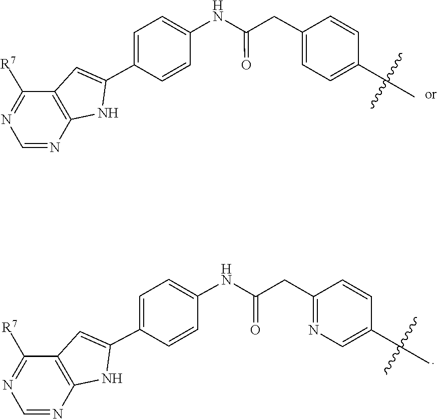

In some particular embodiments, R⁷-Cy¹-Cy²—X—W—Y-Cy³ is:

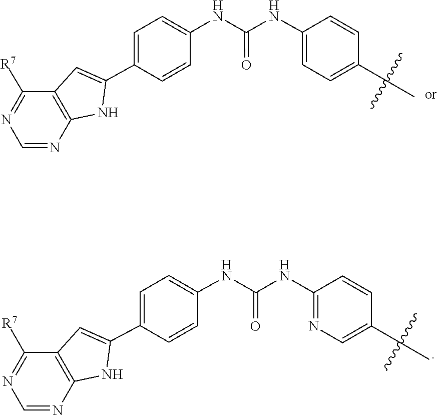

In some particular embodiments, R⁷-Cy¹-Cy²—X—W—Y-Cy³ is:

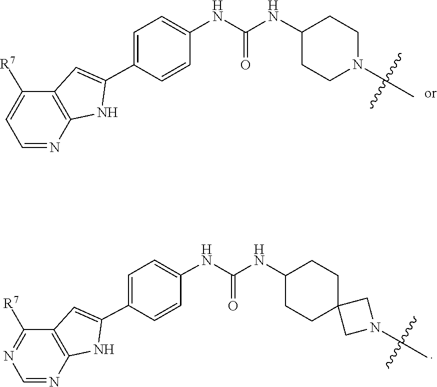

In some particular embodiments, R⁷-Cy¹-Cy²—X—W—Y-Cy³ is:

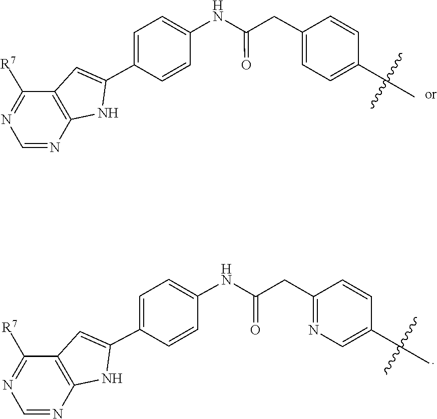

In some particular embodiments, R⁷-Cy¹-Cy²—X—W—Y-Cy³ is:

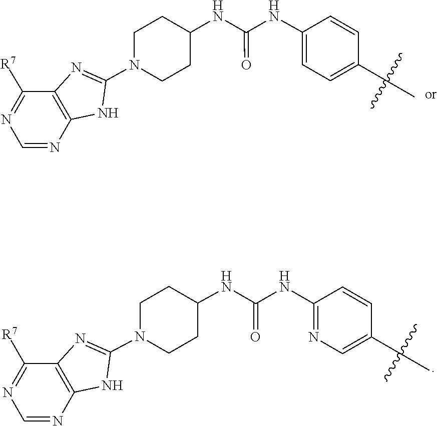

In some particular embodiments, R⁷-Cy¹-Cy²—X—W—Y-Cy³ is:

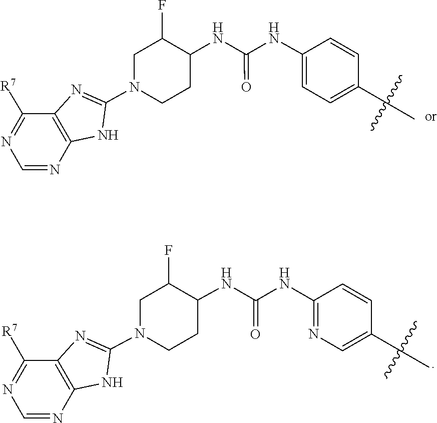

In some particular embodiments, R⁷-Cy¹-Cy²—X—W—Y-Cy³ is:

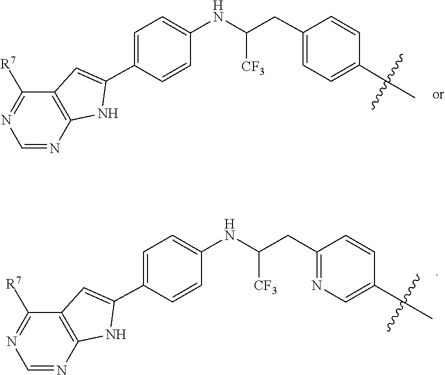

In some particular embodiments, R⁷-Cy¹-Cy²—X—W—Y-Cy³ is:

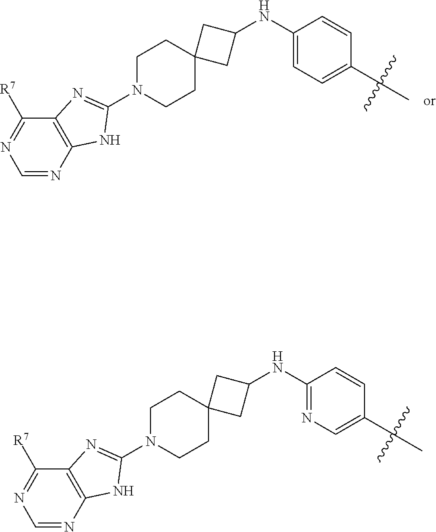

In some particular embodiments, R⁷-Cy¹-Cy²—X—W—Y-Cy³ is:

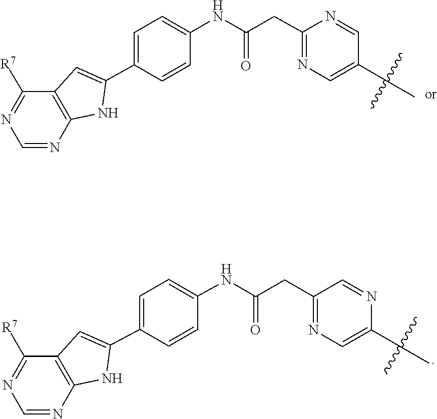

In some particular embodiments, R⁷-Cy¹-Cy²—X—W—Y-Cy³ is:

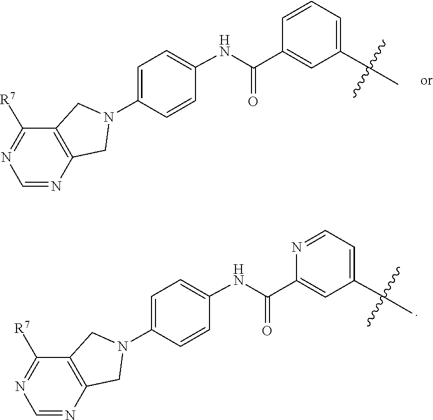

In some particular embodiments, R⁷-Cy¹-Cy²—X—W—Y-Cy³ is:

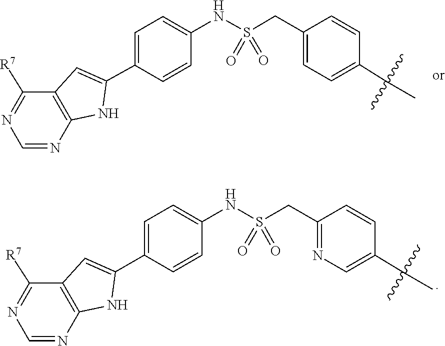

In some embodiments, R⁷ is 4-10 membered heterocycloalkyl ring having 1-2 heteroatoms independently selected from nitrogen, oxygen, or sulfur substituted with Me, Et, or i-Pr. In some embodiments, R⁷ is substituted with methyl. In some embodiments, R⁷ is substituted with cyclopropyl. In some embodiments, R⁷ is fused with cyclopropyl.

In some embodiments, R⁷ is a 5-6 membered heteroaryl ring having 1-4 heteroatoms independently selected from nitrogen, oxygen, and sulfur.

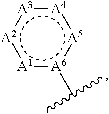

In some embodiments, R⁷ is wherein each A¹, A², A³, A⁴, and A⁵ is independently CR⁸R⁹, NR₈, CR⁸, N, O, or SO₂; A⁶ is CR⁸, C, or N; each R⁸ and R⁹ is independently H or alkyl, and any pair of R⁸ or R⁹ may join to form one or more additional rings including fused, bridged, and bicyclic rings; the R⁷ ring comprises zero, one, or two double bonds, or is aromatic. In some embodiments, when A³ is O and A⁶ is N, then at least one of A¹, A², A⁴, and A⁵ is other than-CH₂—. In some embodiments, A³ is NR₈ and A⁶ is N. In some embodiments, A³ is O and A⁶ is CR⁸. In some embodiments, A³ is CR⁸ and A⁶ is N. In some embodiments, one or more of A¹, A², A³, A⁴, and A⁵ is CF₂. In some embodiments, A¹ is CF₂. In some embodiments, A³ is CF₂. In some embodiments, one or more of A¹, A², A³, A⁴, and AS is CF₂. In some embodiments, A¹ is SO₂. In some embodiments, A³ is SO₂.

In some embodiments, R⁷ is pyrrolidinyl, piperidinyl, piperazinyl, or morpholinyl.

In some embodiments, R⁷ is morpholinyl.

In some embodiments, R⁷ is substituted or unsubstituted heteroaryl.

In some embodiments, R⁷ is substituted or unsubstituted pyridyl or pyrimidyl.

In some embodiments, R⁷ is unsubstituted pyridyl.

In some embodiments, R⁷ is pyridyl substituted with halo, hydroxyl, CN, substituted or unsubstituted C₁₋₆ alkyl, substituted or unsubstituted amino, or substituted or unsubstituted alkoxy.

In some embodiments, R⁷ is pyridyl substituted with Me, Et, i-Pr, OH, Cl, F, CF₃, CN, or NH₂.

In some embodiments, R⁷ is pyridyl substituted with Me, Et, i-Pr, Cl, F, CF₃, or CN.

In some embodiments, R⁷ is substituted or unsubstituted pyrrolyl, pyrazolyl, imidazolyl, oxazolyl, triazolyl, thiazolyl, oxadiazolyl, or thiadiazolyl.

In some embodiments, R⁷ is substituted or unsubstituted imidazolyl.

In some embodiments, R⁷ is imidazoyl substituted with Me, Et, i-Pr, Cl, F, CF₃, or CN.

In some embodiments, R⁷ is imidazoyl substituted with Me.

In some embodiments, R⁷ is substituted or unsubstituted alkyl.

In some embodiments, R⁷ is Me, Et, n-Pr, i-Pr, or n-Bu.

In some embodiments, R⁷ is Me, or Et substituted with halo or hydroxyl.

In some embodiments, R⁷ is Me, or Et substituted with Cl or F.

In some embodiments, R⁷ is Mc, or CF₃.

In some embodiments, R⁷ is 4-10 membered heterocycloalkyl ring; and the heterocyclic ring is a spiro ring. In some embodiments, the spiro ring is 6:3, 6:4, or 6:5 spiro ring; wherein 6:3 spiro ring is a spiro ring where one ring is 6-membered and other is 3-membered ring; 6:4 spiro ring is a spiro ring where one ring is 6-membered and other is 4-membered ring; and 6:5 spiro ring is a spiro ring where one ring is 6-membered and other is 5-membered ring.

In some embodiments, R⁷ is

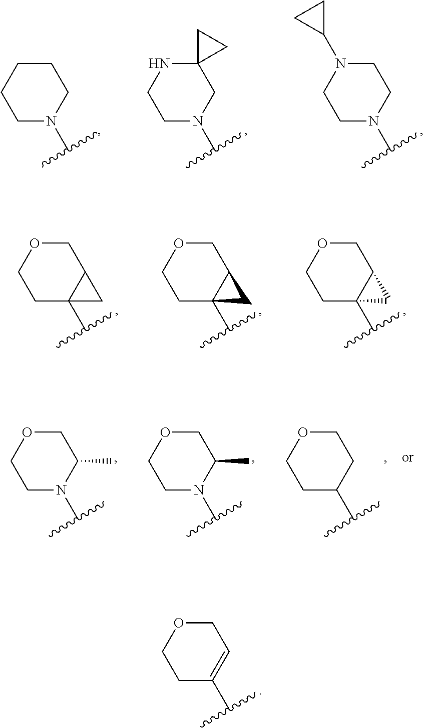

In some embodiments, R⁷ is a spiro ring, and the spiro ring is:

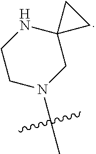 [SMILES: *N1CCNC2(CC2)C1]

In some embodiments, R⁷ is 4-10 membered heterocycloalkyl ring; and the heterocyclic ring is a fused ring. In some embodiments, the fused ring is 6:3, 6:4, or 6:5 ring.

In some embodiments, R⁷ is a fused ring, and the fused ring is:

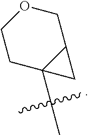 [SMILES: *C12CCOCC1C2]

In some embodiments, R⁷ is 4-10 membered heterocycloalkyl ring; and the heterocyclic ring is a partially unsaturated ring. In some embodiments, the partially saturated ring has a double bond. In some embodiments, R⁷ is an partially saturated ring, and the partially saturated ring is:

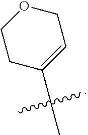 [SMILES: *C1=CCOCC1]

In some embodiments, R⁷ is

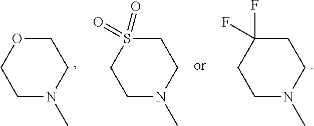

In some embodiments, R⁷ is

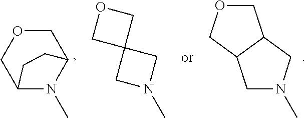

In some embodiments, R⁷ is

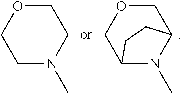

In some embodiments, R⁷ is

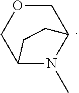

In some embodiments, R⁷ is morpholinyl, piperidinyl, thiomorpholinyl; each unsubstituted or substituted with Me, Et, F, (F)₂, (O)₂, or Cl.

In some particular embodiments, L is absent.

In some particular embodiments, L is —CH₂—, —NH—, —C(D)₂-, —C(F₂)—O—, or —CH₂—CH₂—.

In some embodiments, when present the substituted of substituted or unsubstituted group is the group independently substituted with one or more of alkyl, hydroxyl, alkoxy, trifluroalkyl, trifluoroalkoxy, or halo

In some embodiments, when present substituted Cy¹, Cy², Cy³, or Cy⁴ is Cy¹, Cy², Cy³, or Cy⁴ independently substituted with one or more of alkyl, hydroxyl, alkoxy, trifluroalkyl, trifluoroalkoxy, or halo.

In some embodiments, when present substituted Cy¹, Cy², Cy³, or Cy⁴ is Cy¹, Cy², Cy³, or Cy⁴ independently substituted with one or more of Me, Et, i-Pr, CF₃, OMe, OEt, OCF₃, F, or Cl.

In one embodiment, R^{3c} is H. In another embodiment, R^{3c} is Me.

In some particular embodiments, Cy¹-Cy²—X—W—Y-Cy³ is:

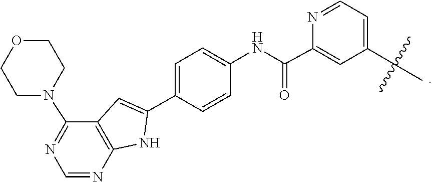 [SMILES: *c1ccnc(C(=O)Nc2ccc(-c3cc4c(N5CCOCC5)ncnc4[nH]3)cc2)c1]

In some particular embodiments, Cy¹-Cy²—X—W—Y-Cy³ is:

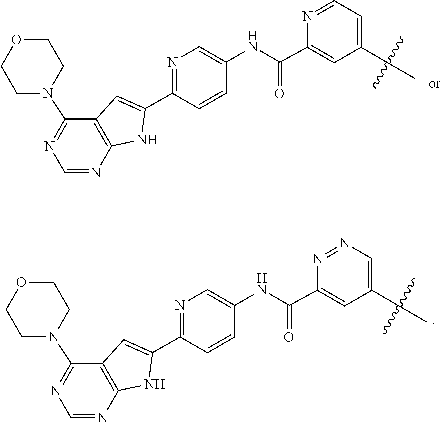 [SMILES: *c1ccnc(C(=O)Nc2ccc(-c3cc4c(N5CCOCC5)ncnc4[nH]3)nc2)c1 ; *c1cnnc(C(=O)Nc2ccc(-c3cc4c(N5CCOCC5)ncnc4[nH]3)nc2)c1]

In some particular embodiments, Cy¹-Cy²—X—W—Y-Cy³ is:

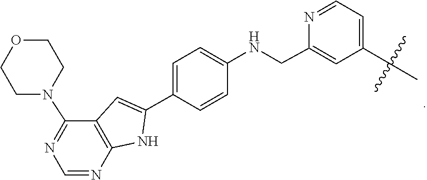 [SMILES: *c1ccnc(CNc2ccc(-c3cc4c(N5CCOCC5)ncnc4[nH]3)cc2)c1]

In some particular embodiments, Cy¹-Cy²—X—W—Y-Cy³ is:

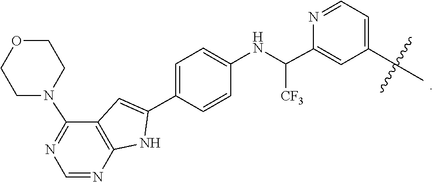 [SMILES: *c1ccnc(C(Nc2ccc(-c3cc4c(N5CCOCC5)ncnc4[nH]3)cc2)C(F)(F)F)c1]

In some particular embodiments, Cy¹-Cy²—X—W—Y-Cy³ is:

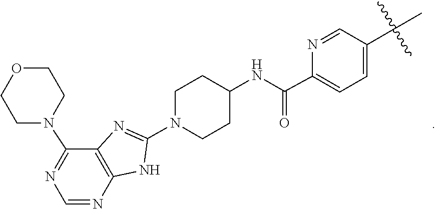 [SMILES: *c1ccc(C(=O)NC2CCN(c3nc4c(N5CCOCC5)ncnc4[nH]3)CC2)nc1]

In some particular embodiments, Cy¹-Cy²—X—W—Y-Cy³ is:

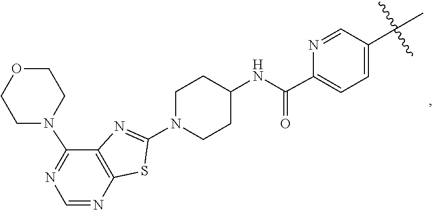 [SMILES: *c1ccc(C(=O)NC2CCN(c3nc4c(N5CCOCC5)ncnc4s3)CC2)nc1]

In some particular embodiments, Cy¹-Cy²—X—W—Y-Cy³ is:

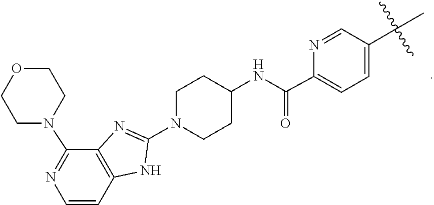 [SMILES: *c1ccc(C(=O)NC2CCN(c3nc4c(N5CCOCC5)nccc4[nH]3)CC2)nc1]

In some particular embodiments, Cy¹-Cy²—X—W—Y-Cy³ is:

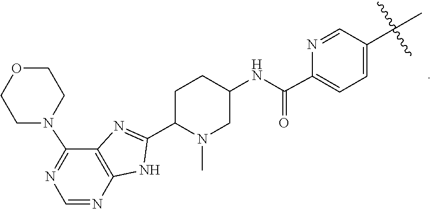 [SMILES: *c1ccc(C(=O)NC2CCC(c3nc4c(N5CCOCC5)ncnc4[nH]3)N(C)C2)nc1]

In some particular embodiments, Cy¹-Cy²—X—W—Y-Cy³ is:

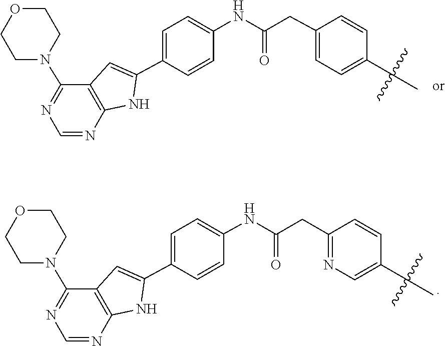 [SMILES: *c1ccc(CC(=O)Nc2ccc(-c3cc4c(N5CCOCC5)ncnc4[nH]3)cc2)cc1 ; *c1ccc(CC(=O)Nc2ccc(-c3cc4c(N5CCOCC5)ncnc4[nH]3)cc2)nc1]

In some particular embodiments, Cy¹-Cy²—X—W—Y-Cy³ is:

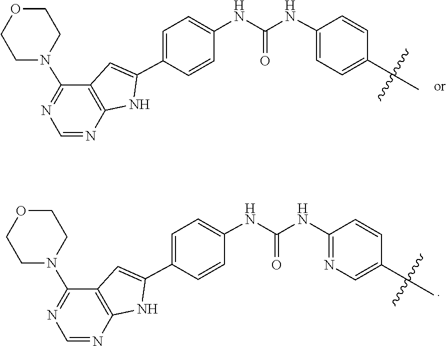 [SMILES: *c1ccc(NC(=O)Nc2ccc(-c3cc4c(N5CCOCC5)ncnc4[nH]3)cc2)cc1 ; *c1ccc(NC(=O)Nc2ccc(-c3cc4c(N5CCOCC5)ncnc4[nH]3)cc2)nc1]

In some particular embodiments, Cy¹-Cy²—X—W—Y-Cy³ is:

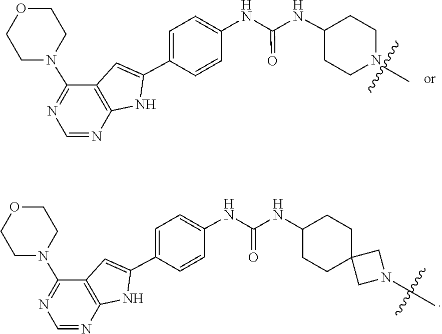

In some particular embodiments, Cy¹-Cy²—X—W—Y-Cy³ is:

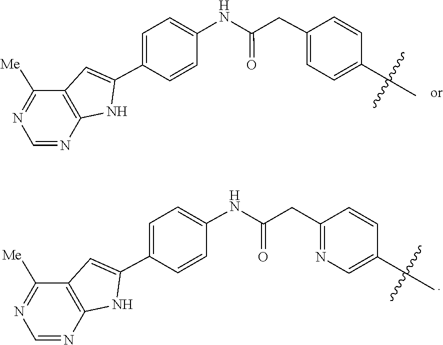 [SMILES: *c1ccc(CC(=O)Nc2ccc(-c3cc4c(C)ncnc4[nH]3)cc2)cc1 ; *c1ccc(CC(=O)Nc2ccc(-c3cc4c(C)ncnc4[nH]3)cc2)nc1]

In some particular embodiments, Cy¹-Cy²—X—W—Y-Cy³ is:

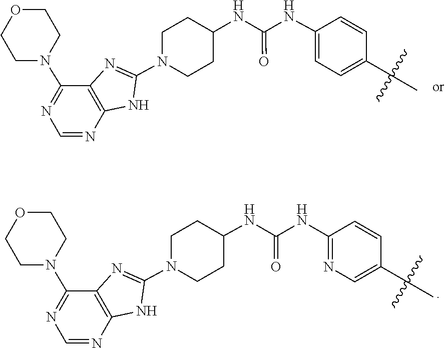 [SMILES: *c1ccc(NC(=O)NC2CCN(c3nc4c(N5CCOCC5)ncnc4[nH]3)CC2)cc1 ; *c1ccc(NC(=O)NC2CCN(c3nc4c(N5CCOCC5)ncnc4[nH]3)CC2)nc1]

In some particular embodiments, Cy¹-Cy²—X—W—Y-Cy³ is:

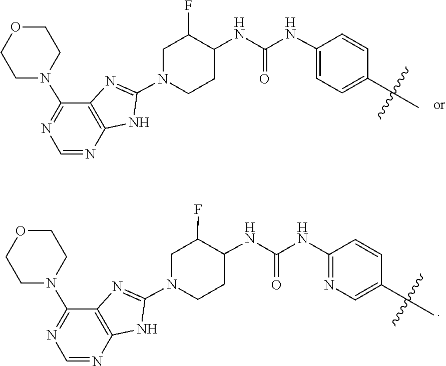 [SMILES: *c1ccc(NC(=O)NC2CCN(c3nc4c(N5CCOCC5)ncnc4[nH]3)CC2F)cc1 ; *c1ccc(NC(=O)NC2CCN(c3nc4c(N5CCOCC5)ncnc4[nH]3)CC2F)nc1]

In some particular embodiments, Cy¹-Cy²—X—W—Y-Cy³ is:

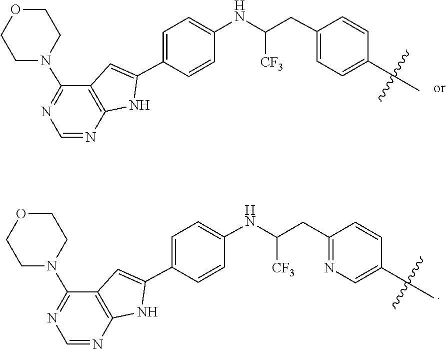 [SMILES: *c1ccc(CC(Nc2ccc(-c3cc4c(N5CCOCC5)ncnc4[nH]3)cc2)C(F)(F)F)cc1 ; *c1ccc(CC(Nc2ccc(-c3cc4c(N5CCOCC5)ncnc4[nH]3)cc2)C(F)(F)F)nc1]

In some particular embodiments, Cy¹-Cy²—X—W—Y-Cy³ is:

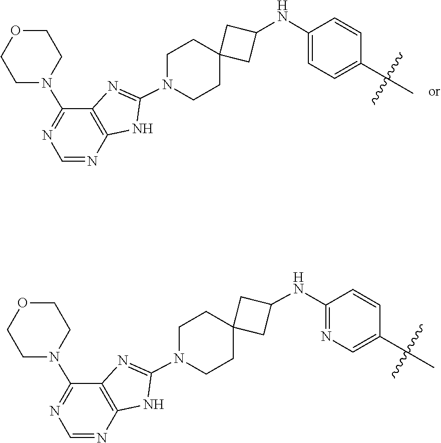 [SMILES: *c1ccc(NC2CC3(CCN(c4nc5c(N6CCOCC6)ncnc5[nH]4)CC3)C2)cc1 ; *c1ccc(NC2CC3(CCN(c4nc5c(N6CCOCC6)ncnc5[nH]4)CC3)C2)nc1]

In some particular embodiments, Cy¹-Cy²—X—W—Y-Cy³ is:

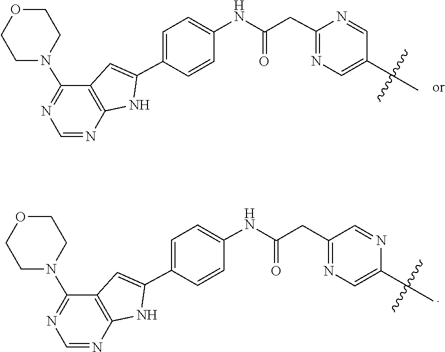 [SMILES: *c1cnc(CC(=O)Nc2ccc(-c3cc4c(N5CCOCC5)ncnc4[nH]3)cc2)nc1 ; *c1cnc(CC(=O)Nc2ccc(-c3cc4c(N5CCOCC5)ncnc4[nH]3)cc2)cn1]

In some particular embodiments, Cy¹-Cy²—X—W—Y-Cy³ is:

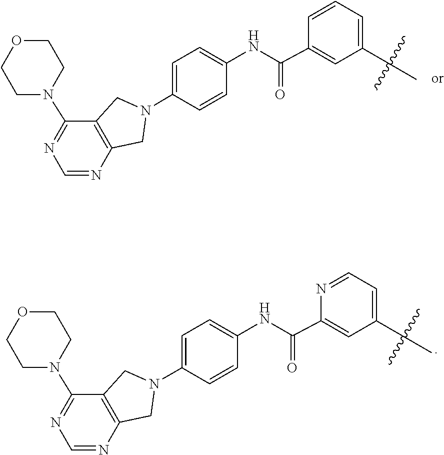 [SMILES: *c1cccc(C(=O)Nc2ccc(N3Cc4ncnc(N5CCOCC5)c4C3)cc2)c1 ; *c1ccnc(C(=O)Nc2ccc(N3Cc4ncnc(N5CCOCC5)c4C3)cc2)c1]

In some particular embodiments, Cy¹-Cy²—X—W—Y-Cy³ is:

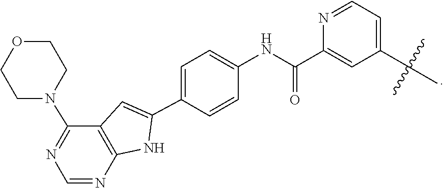 [SMILES: *c1ccnc(C(=O)Nc2ccc(-c3cc4c(N5CCOCC5)ncnc4[nH]3)cc2)c1]

In some particular embodiments, Cy¹-Cy²—X—W—Y-Cy³ is:

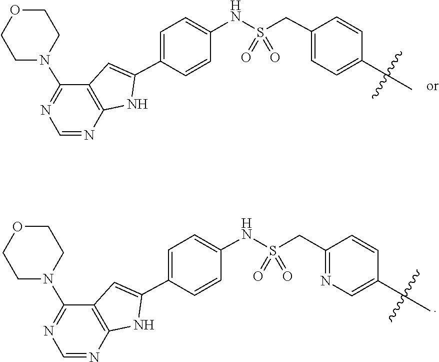 [SMILES: *c1ccc(CS(=O)(=O)Nc2ccc(-c3cc4c(N5CCOCC5)ncnc4[nH]3)cc2)cc1 ; *c1ccc(CS(=O)(=O)Nc2ccc(-c3cc4c(N5CCOCC5)ncnc4[nH]3)cc2)nc1]

In some particular embodiments, Cy⁴ is absent.

In some particular embodiments, L-Cy⁴-R¹ is:

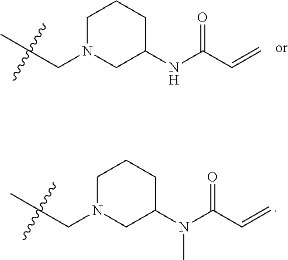 [SMILES: *CN1CCCC(NC(=O)C=C)C1 ; *CN1CCCC(N(C)C(=O)C=C)C1]

In some particular embodiments, L-Cy⁴-R¹ is:

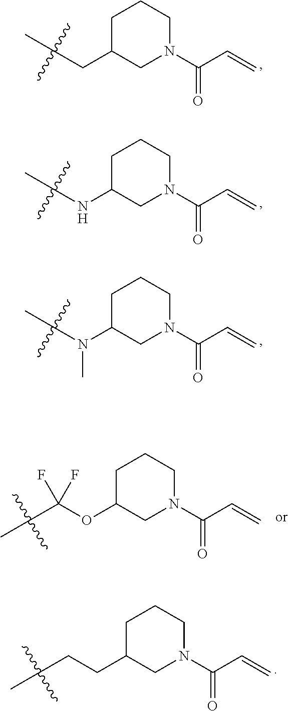 [SMILES: *CC1CCCN(C(=O)C=C)C1 ; *NC1CCCN(C(=O)C=C)C1 ; *N(C)C1CCCN(C(=O)C=C)C1 ; *C(F)(F)OC1CCCN(C(=O)C=C)C1 ; *CCC1CCCN(C(=O)C=C)C1]

In some particular embodiments, L-Cy⁴-R¹ is:

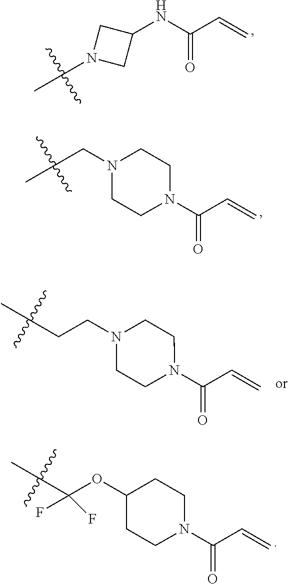

In some particular embodiments, L-Cy⁴-R¹ is:

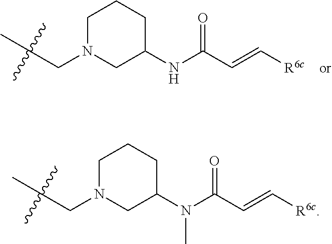

In some particular embodiments, L-Cy⁴-R¹ is:

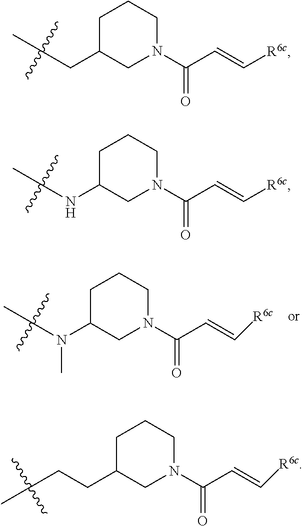

In some particular embodiments, L-Cy⁴-R¹ is:

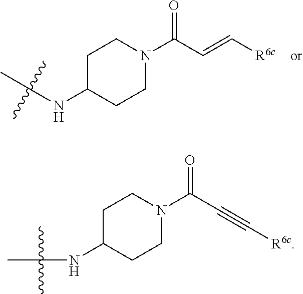

In some particular embodiments, L-Cy⁴-R¹ is:

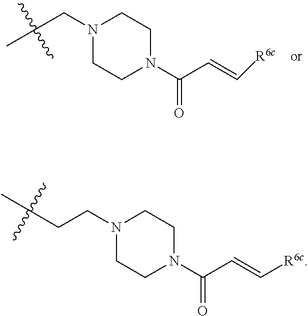

In some particular embodiments, L-Cy⁴-R¹ is:

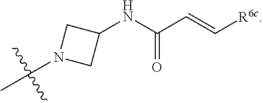

In some particular embodiments, L-Cy⁴-R¹ is:

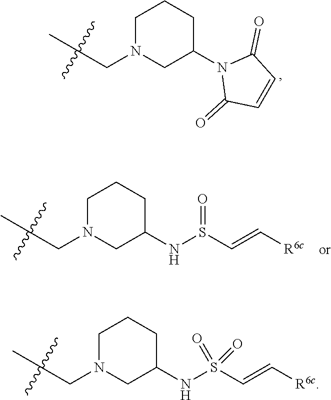

In certain embodiments, Cy¹-Cy²—X—W—Y-Cy³-L-Cy⁴-R¹ is

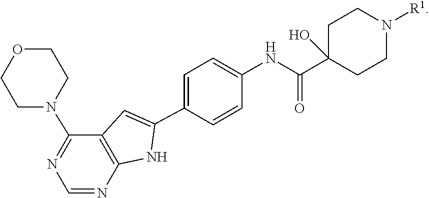

In another aspect, provided herein is a compound according to Formula (L-I) having the structure:

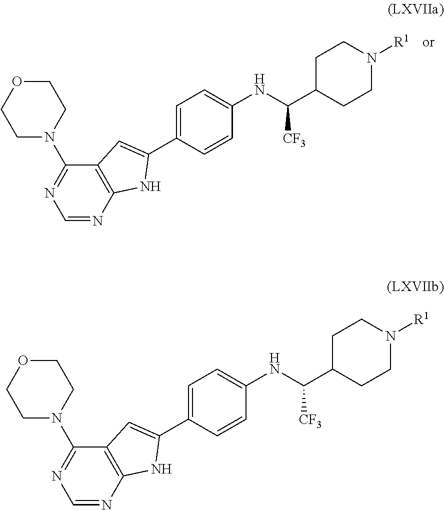

or a stereoisomer thereof; or a pharmaceutically acceptable salt thereof, wherein R¹ is as described herein.

In another aspect, provided herein is a compound according to Formula (L-I) having the structure:

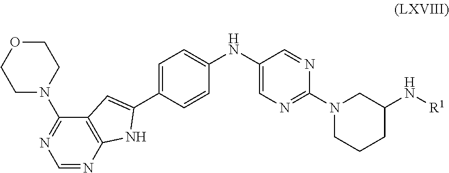

or a stereoisomer thereof; or a pharmaceutically acceptable salt thereof, wherein R¹ is as described herein.

In certain embodiments, the compound is according to formula (LXIXa), (LXIXb), or (LXIXc):

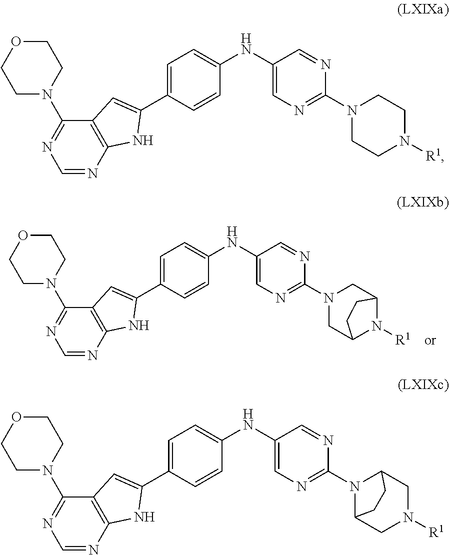

or a stereoisomer thereof; or a pharmaceutically acceptable salt thereof, wherein R¹ is as described herein.

In another aspect, provided herein is a compound according to Formula (L-I) having the structure:

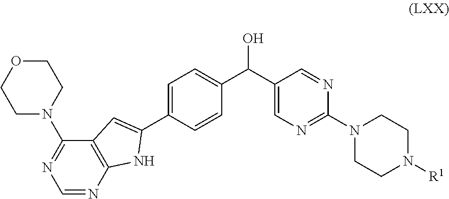

or a stereoisomer thereof; or a pharmaceutically acceptable salt thereof, wherein R¹ is as described herein.

In another aspect, provided herein is a compound according to Formula (L-I) having the structure:

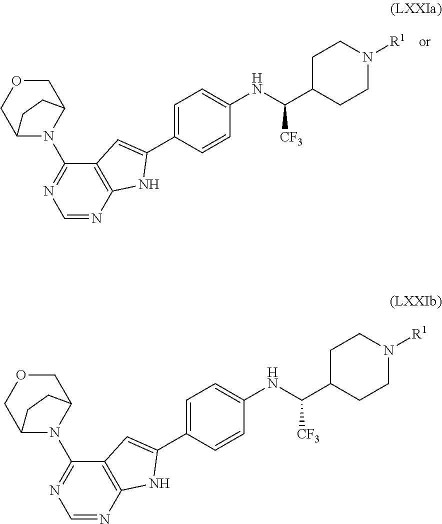

or a stereoisomer thereof; or a pharmaceutically acceptable salt thereof, wherein R¹ is as described herein.

In another aspect, provided herein is a compound according to Formula (L-I) having the structure:

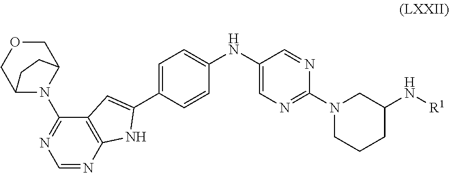

or a stereoisomer thereof; or a pharmaceutically acceptable salt thereof, wherein R¹ is as described herein.

In another aspect, provided herein is a compound according to Formula (L-I) having the structure:

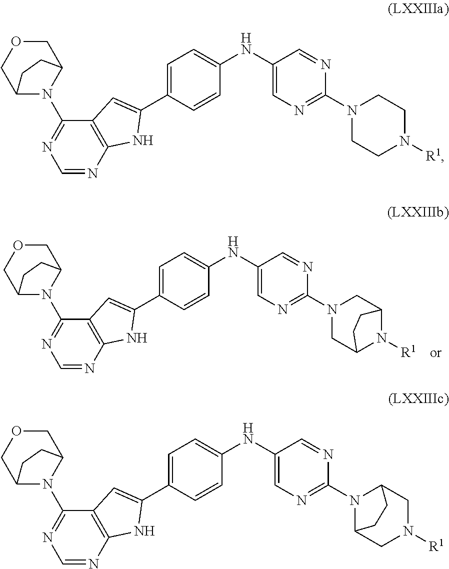

or a stereoisomer thereof; or a pharmaceutically acceptable salt thereof, wherein R¹ is as described herein.

In another aspect, provided herein is a compound according to Formula (L-I) having the structure:

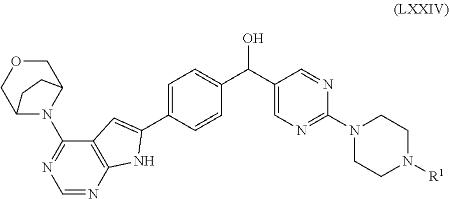

or a stereoisomer thereof; or a pharmaceutically acceptable salt thereof, wherein R¹ is as described herein.

In certain embodiments, the compound is according to formula (LXXVa) or (LXXVb)

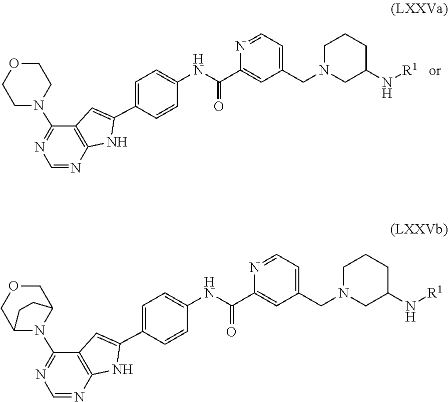

or a stereoisomer thereof; or a pharmaceutically acceptable salt thereof, wherein R¹ is as described herein.

In certain embodiments, the compound is according to formula (LXXVIa), (LXXVIb), (LXXVIc), (LXXVId), (LXXVIe), or (LXXVIf)

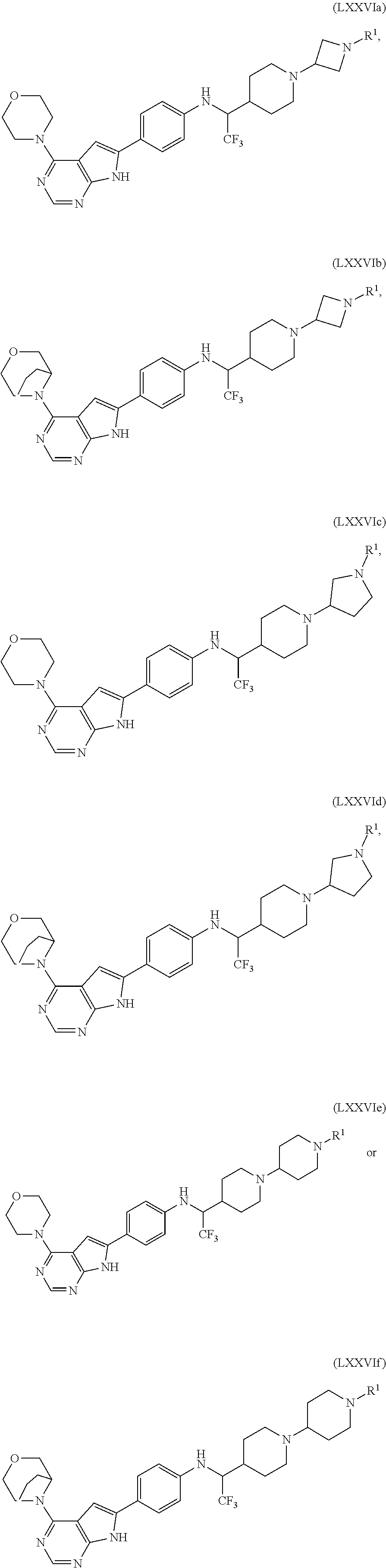

or a stereoisomer thereof; or a pharmaceutically acceptable salt thereof, wherein R¹ is as described herein

In certain embodiments, the compound is according to formula (LXXVIIa), (LXXVIIb), (LXXVIIc), (LXXVIId), (LXXVIIe), or (LXXVIIf)

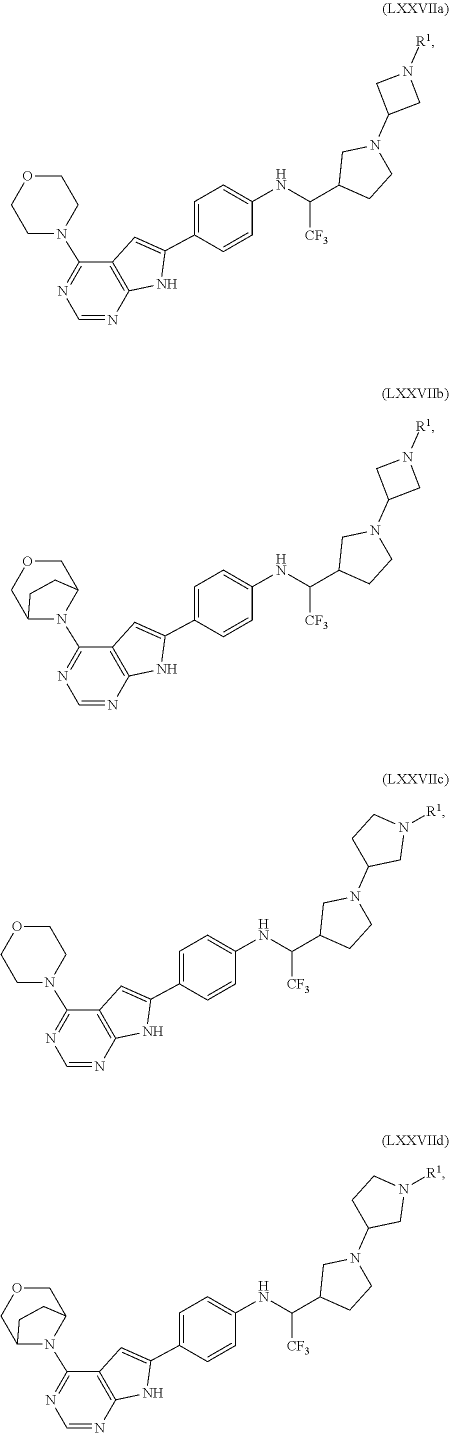

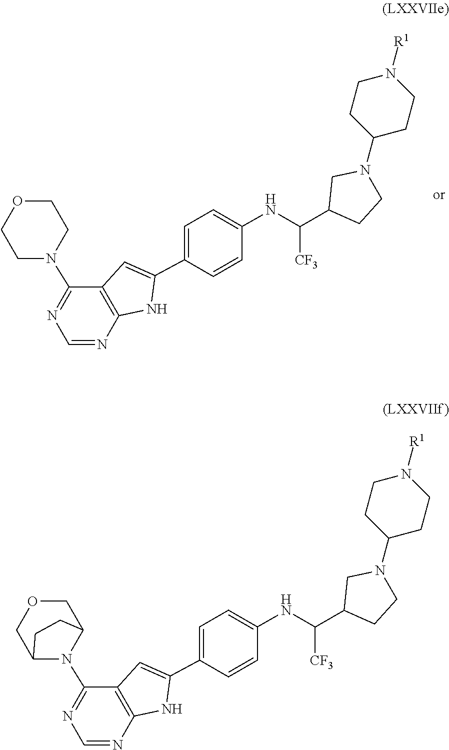

or a stereoisomer thereof; or a pharmaceutically acceptable salt thereof, wherein R¹ is as described herein.

In certain embodiments, the compound is according to formula (LXXVIII)

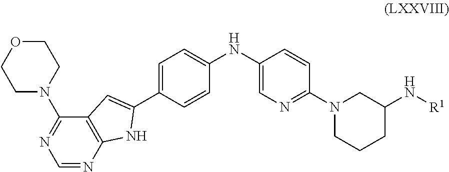

or a stereoisomer thereof; or a pharmaceutically acceptable salt thereof, wherein R¹ is as described herein.

In certain embodiments, R¹ is —C(O)—C(R^{6a})═C(R^{6b})—C(O)—R^{6c}, —S(O)—C(R^{6a})—C(R^{6b})—C(O)—R^{6c}, or —S(O)₂—C(R^{6a})—C(R^{6b}); and Cy⁴ is absent.

In certain embodiments, R¹ is —C(O)—B—C(R^{6a})═C(R^{6b})—C(O)—R^{6c}, —S(O)—B—C(R^{6a})—C(R^{6b})—C(O)—R^{6c}, or —S(O)₂—B—C(R^{6a})═C(R^{6b})—C(O)—R^{6c}. In certain embodiments, R¹ is —B-C(R^{6a})—C(R^{6b})—P(O)—R^{6a}R^{6b}; or —B—C(R^{6a})═C(R^{6b})—P(O)—OR^{6a}OR^{6b}.

In certain embodiments, B is a group as defined herein and not to be mistaken with boron.

In certain embodiments, R¹ is —B-C(R^{6a})═C(R^{6b})—P(O)—R^{6a}R^{6b}.

In certain embodiments, R¹ is B is substituted or unsubstituted C₁₋₄ alkylene.

In certain embodiments, R¹ is B is substituted or unsubstituted cycloalkyl, or substituted or unsubstituted heterocycloalkyl

In certain embodiments, R¹ is —B-C(R^{6a})═C(R^{6b})—C(O)—R^{6c}, —B—C(R^{6a})═C(R^{6b})—S(O)—R^{6c}; —B-C(R^{6a})═C(R^{6b})—S(O)₂—R^{6c}; B is substituted or unsubstituted C₁₋₄ alkylene;

In certain embodiments, R¹ is —B-C(R^{6a})═C(R^{6b})—C(O)—R^{6c}.

In certain embodiments, B is substituted or unsubstituted C₁₋₂ alkylene.

In certain embodiments, B is —CH₂—, or —CH₂—CH₂—.

In certain embodiments, R¹ is —CH₂—C(R^{6a})═C(R^{6b})—C(O)—R^{6c}.

In certain embodiments, each R^{6a} and R^{6b} is independently H, CN, halo, or C₁₋₆ alkyl.

In certain embodiments, each of R^{6a}, and R^{6b} is H.

In certain embodiments, R^{6a} is F.

In certain embodiments, R^{6b} is F.

In certain embodiments, R^{6a} and R^{6b} are joined together to form a bond.

In certain embodiments, R^{6a} is Me, Et, or i-Pr.

In certain embodiments, R^{6a} is substituted alkyl. In certain embodiments, the substitution on alkyl is selected from halo, CN, substituted or unsubstituted alkoxy, substituted or unsubstituted amino, and substituted or unsubstituted heterocycloalkyl. In certain embodiments, the substitution on alkoxy, and heterocycloalkyl is selected from 1, 2, or 3 of C₁-C₄ alkyl, amino, alkylamino, dialkylamino, hydroxy, C₁-C₄ hydroxyalkyl, CN, C₁-C₄ alkoxy, and halo. In certain embodiments, the substitution on amino is selected from 1, or 2 of C₁-C₄ alkyl. In certain embodiments, the substitution on amino is selected from 1, or 2 of haloC₁—C₄ alkyl.

In certain embodiments, R^{6a} is —CH₂—CN, —CH₂—F, —CH₂—S(O)₂Me, —CH₂-morpholin-1-yl, —CH₂-pyrrolidin-1-yl, —CH₂-piperidin-1-yl, —CH₂—OMe, —CH₂—NHMe, —CH₂—NMe₂, —CH₂—N(H)Ac, CF₃, Cl, or F.

In certain embodiments, R¹ is —CH₂—C≡C—C(O)—R^{6c}. In certain embodiments, R¹ is —CH₂—CH═CH—C(O)—R^{6c}.

In certain embodiments, Cy¹-Cy²—X—W—Y-Cy³-L-Cy⁴-R¹ is

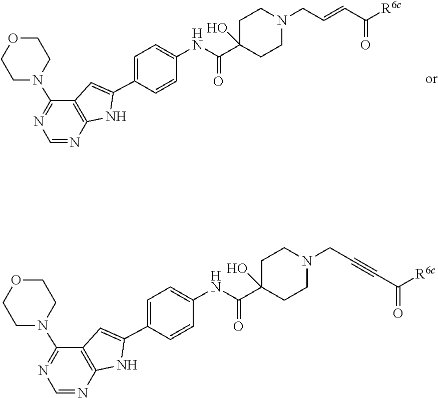

In certain embodiments, the compound is according to formula LIa, LIb, LIc, LId, LIe or LIf:

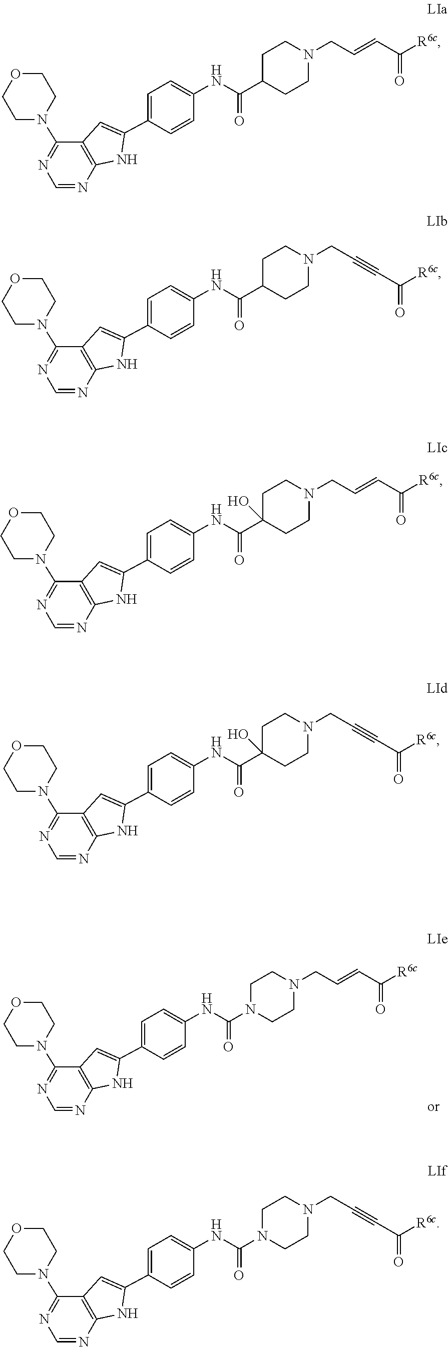

In certain embodiments, R^{6c} is substituted or unsubstituted alkoxy. In certain embodiments, R^{6c} is substituted alkoxy; and the substitution is selected from C₁-C₄ alkyl, and halo. In certain embodiments, R^{6c} is substituted alkoxy; and the substitution is selected from C₁-C₄ alkyl.

In certain embodiments, R^{6c} is OMe, OEt, O-i-Pr, or O-t-Bu.

In certain embodiments, R^{6c} is OMe, OEt, O-i-Pr, or O-t-Bu.

In certain embodiments, Roc is heterocycloalkyl; unsubstituted or substituted with Me, Et, F, (F)₂, or Cl.

In certain embodiments, R^{6c} is substituted or unsubstituted heterocycloalkyl.

In certain embodiments, R^{6c} is substituted or unsubstituted azetidinyl, pyrrolidinyl, piperidinyl, or azepinyl.

In certain embodiments, R^{6c} is azetidin-1-yl, pyrrolidin-1-yl, piperidin-1-yl, or azepin-1-yl. In certain embodiments, R^{6c} is azetidin-1-yl, pyrrolidin-1-yl, piperidin-1-yl, or azepin-1-yl; each unsubstituted or substituted with Me, Et, F, (F)₂, or Cl. In certain embodiments, piperidin-1-yl or azetidine-1-yl; each unsubstituted or substituted with Me, Et, F, (F)₂, or Cl.

In certain embodiments, R^{6c} is piperidin-1-yl.

In certain embodiments, R^{6c} is substituted or unsubstituted amino.

In certain embodiments, R^{6c} is substituted amino. In certain embodiments, R^{6c} is substituted amino; and the substitution is C₁-C₄ alkyl or C₁-C₄ haloalkyl. In certain embodiments, R^{6c} is —N(Me) CF₃ or —N(Me) CH₂—CF₃.

In certain embodiments, R^{6c} is dialkylamino.

In certain embodiments, R^{6c} is dimethylamino, diethylamino, N-isopropyl-N-methylamino, or N-isopropyl-N-ethylamino.

In certain embodiments, R^{6c} is dimethylamino.

In particular embodiments, R^{6c} is —O-t-Bu, —NMe₂, or N(Me)CF₃. In further particular embodiments, R^{6c} is heterocycloalkyl. In further particular embodiments, R^{6c} is heterocycloalkyl, substituted with 1 or 2 halo, alkyl, haloalkyl, or alkoxy. In further particular embodiments, R^{6c} is 1-azetidinyl, 1-pyrrolidinyl, 1-piperidinyl, or 1-piperazinyl. In further particular embodiments, R^{6c} is 1-azetidinyl, 1-pyrrolidinyl, 1-piperidinyl, or 1-piperazinyl, and each of which substituted independently with 1 or 2 halo, alkyl, haloalkyl, or alkoxy. In further particular embodiments, R^{6c} is 1-azetidinyl, substituted with F or difluoro.

In certain embodiments, R^{6c} is —CH₂—OH. In certain embodiments, R^{6c} is —OH.

In certain embodiments, Cy¹-Cy²—X—W—Y-Cy³-L-Cy⁴-R¹ is

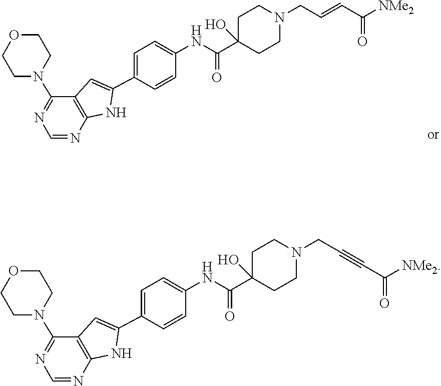 [SMILES: CN(C)C(=O)/C=C/CN1CCC(O)(C(=O)Nc2ccc(-c3cc4c(N5CCOCC5)ncnc4[nH]3)cc2)CC1 ; CN(C)C(=O)C#CCN1CCC(O)(C(=O)Nc2ccc(-c3cc4c(N5CCOCC5)ncnc4[nH]3)cc2)CC1]

In certain embodiments, the compound is according to formula LIIa, LIIb, LIIc, LIId, LIIe or LIIf:

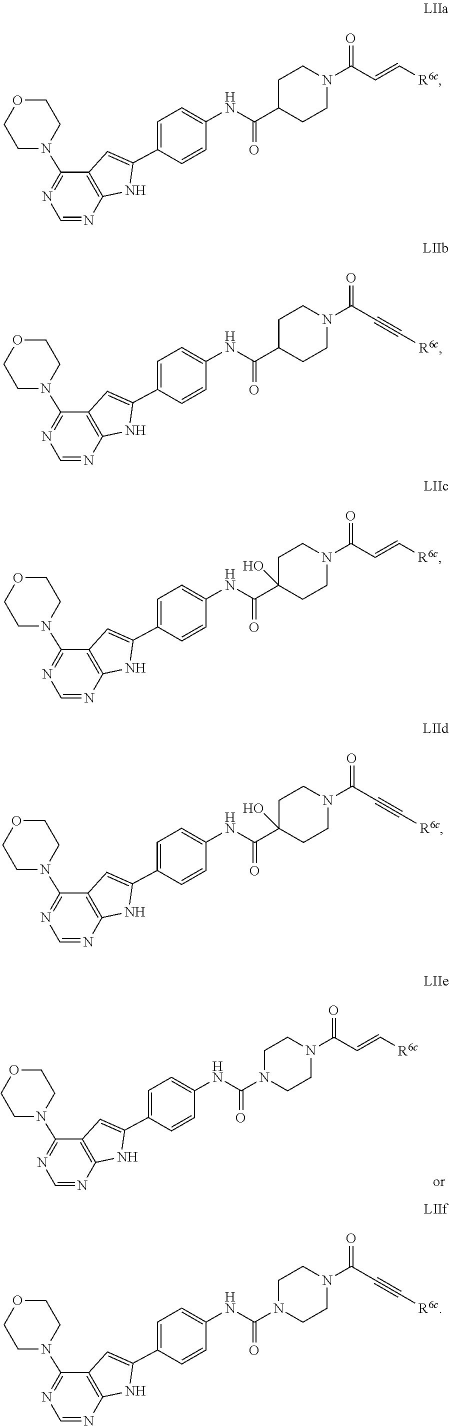

In certain embodiments, R^{6c} is H, or C₁-C₄ alkyl substituted with heterocycloalkyl; unsubstituted or substituted with Me, Et, F, (F)₂, or Cl.

In certain embodiments, R^{6c} is H.

In certain embodiments, Cy¹-Cy²—X—W—Y-Cy³-L-Cy⁴-R¹ is

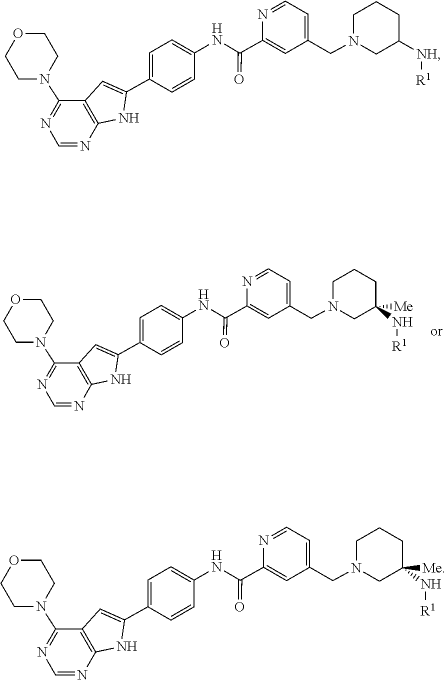

In certain embodiments, Cy¹-Cy²—X—W—Y-Cy³-L-Cy⁴-R¹ is

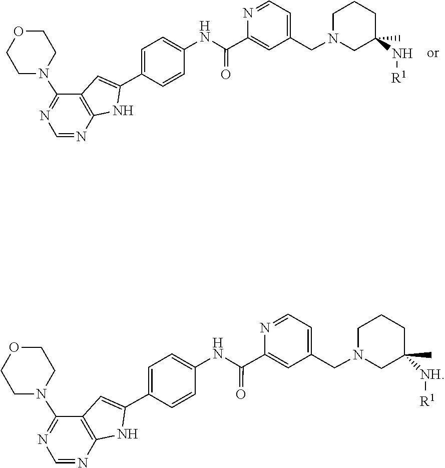

In certain embodiments, Cy¹-Cy²—X—W—Y-Cy³-L-Cy⁴-R¹ is

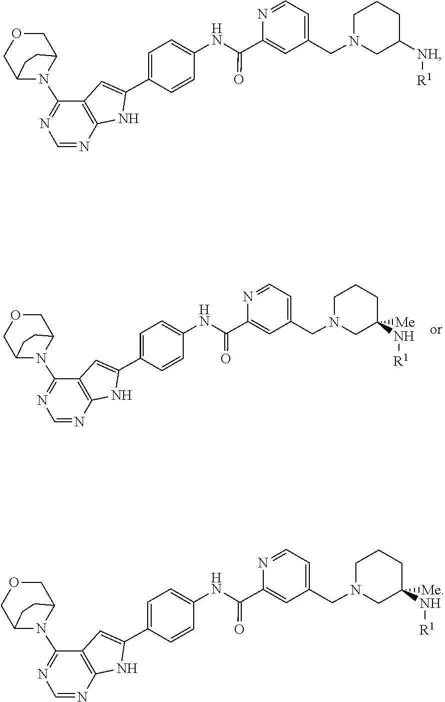

In certain embodiments, Cy¹-Cy²—X—W—Y-Cy³-L-Cy⁴-R¹ is

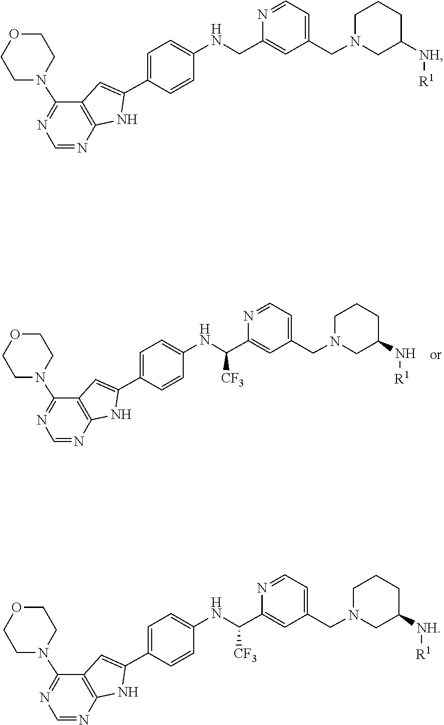

In certain embodiments, Cy¹-Cy²—X—W—Y-Cy³-L-Cy⁴-R¹ is

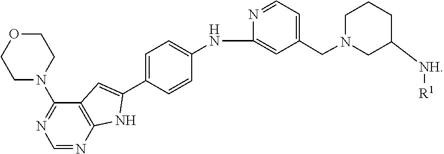

In certain embodiments, Cy¹-Cy²—X—W—Y-Cy³-L-Cy⁴-R¹ is

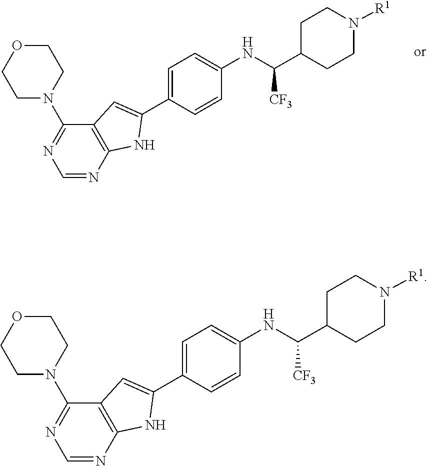

In certain embodiments, Cy¹-Cy²—X—W—Y-Cy³-L-Cy⁴-R¹ is

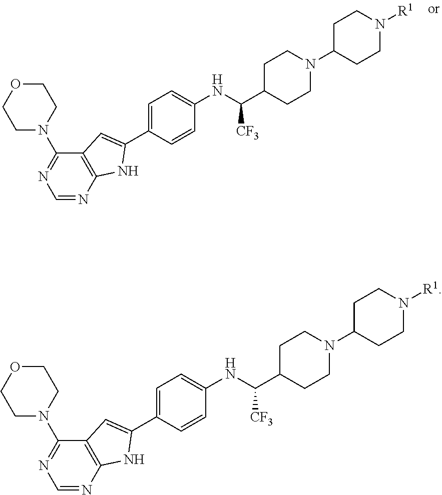

In certain embodiments, Cy¹-Cy²—X—W—Y-Cy³-L-Cy⁴-R¹ is

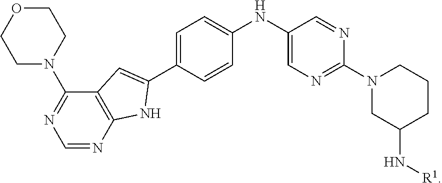

In certain particular embodiments, R⁷ is

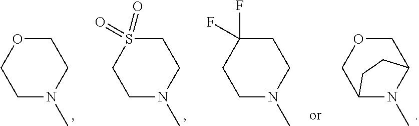

In more particular embodiments, R⁷ is

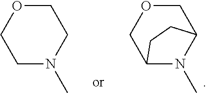

In particular embodiments, R¹ is —S(O)₂—CH═CH₂, —NH—C(O)—C≡CH, —C(O)—CH═CH₂, —C(O)—C≡CH, —NH—C(O)—CH═CH₂, —C(O)—C≡C-Me, or —NH—S(O)₂—CH═CH₂.

In particular embodiments, R¹ is —C(O)—CH—CH—CH₂OH, or —C(O)—CH—CH—OH.

In more particular embodiments, R¹ is —NH—C(O)—C(Cl)=CH₂, —NH—C(O)—C(F)—CH₂, —NH—C(O)—C(CH₂—NMe₂)=CH₂, —NH—S(O)₂—C(Me)=CH₂, —NH—C(O)—C(CH₂-morpholin-1-yl)=CH₂, or —NH—C(O)—C(CH₂—OMe)=CH₂. In more particular embodiments, R¹ is —NH—C(O)—C(CH₂-morpholin-1-yl)=CH₂.

In particular embodiments, R¹ is —NH—C(O)—C≡C—CH₂—NMe₂, —NH—C(O)—C(H)═C(H)—CH₂—NMe₂, —NH—C(O)—C(H)═C(H)—CH₂-(3,3-difluoroazetidin-1-yl), —NH—C(O)—C≡C-Me, —NH—C(O)—C(CH₂—SO₂Me)—CH₂, —NH—C(O)—C(CH₂—CH₂—NMe₂)=CH₂, —NH—C(O)—C(CH₂-(3,3-difluoroazetidin-1-yl))=CH₂, —NH—C(O)—C(CH₂-(3-F-3-Me-azetidin-1-yl))=CH₂, —NH—C(O)—C(CH₂—N(Me) (t-Bu))=CH₂, —NH—C(O)—C≡C—CH₂-(3,3-difluoroazetidin-1-yl), —NH—C(O)—C(CH₂-(3-F-azetidin-1-yl))=CH₂, —NH—C(O)—C(CH₂-(3-F-3-hydroxymethyl-azetidin-1-yl))—CH₂, or —NH—C(O)—C(H)═C(H)—CH₂—OMe.

In particular embodiments, R¹ is —C(O)—C≡C—CH₂—NMe₂, —C(O)—C(H)═C(H)—CH₂—NMe₂, —C(O)—C(H)═C(H)—CH₂-(3,3-difluoroazetidin-1-yl), —C(O)—C≡C-Me, —C(O)—C(CH₂—SO₂Me)=CH₂, —C(O)—C(CH₂—CH₂—NMe₂)=CH₂, —C(O)—C(CH₂-(3,3-difluoroazetidin-1-yl))=CH₂, —C(O)—C(CH₂-(3-F-3-Me-azetidin-1-yl))=CH₂, —C(O)—C(CH₂—N(Me) (t-Bu))=CH₂, —C(O)—C≡C—CH₂-(3,3-difluoroazetidin-1-yl), —C(O)—C(CH₂-(3-F-azetidin-1-yl))=CH₂, —C(O)—C(CH₂-(3-F-3-hydroxymethyl-azetidin-1-yl))=CH₂, —C(O)—C(H)═C(H)—CH₂—OMe, or —C(O)—C≡C—CH₂—OMe.

In certain embodiments, R¹ is —CH₂—C≡C—C(O)—NMe₂. In certain embodiments, R¹ is C(O)—C≡C—CH₂—OMe.

In certain embodiments, R¹ is

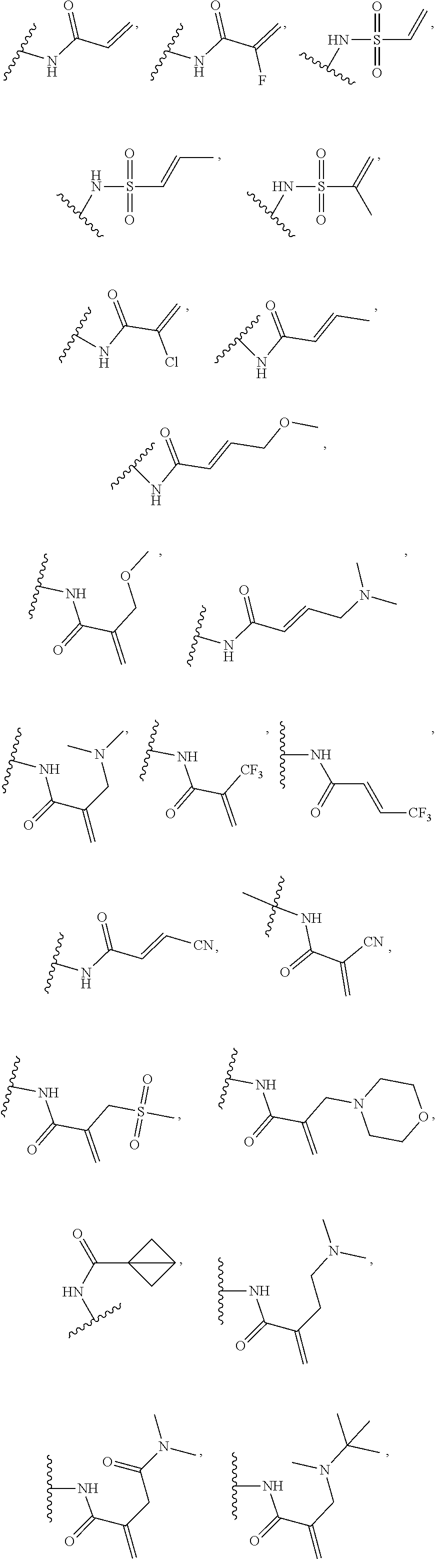

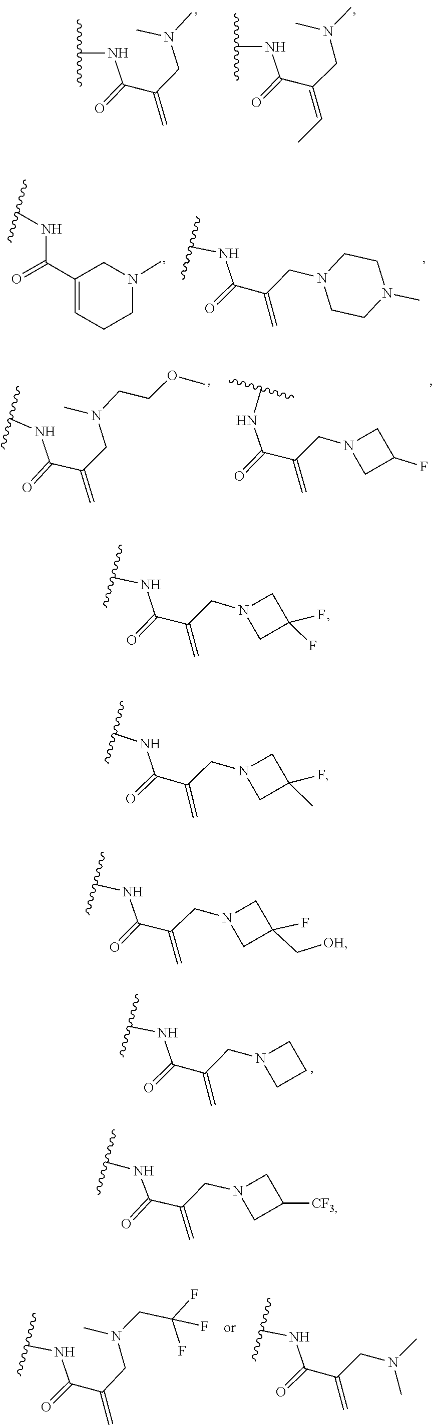

In particular embodiments, R¹ is

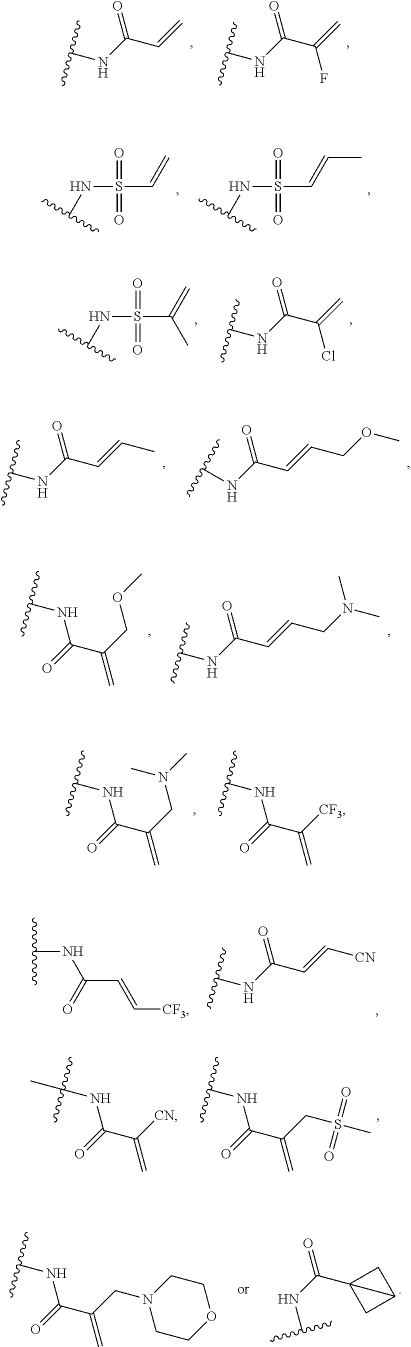

In certain embodiments, R¹ is

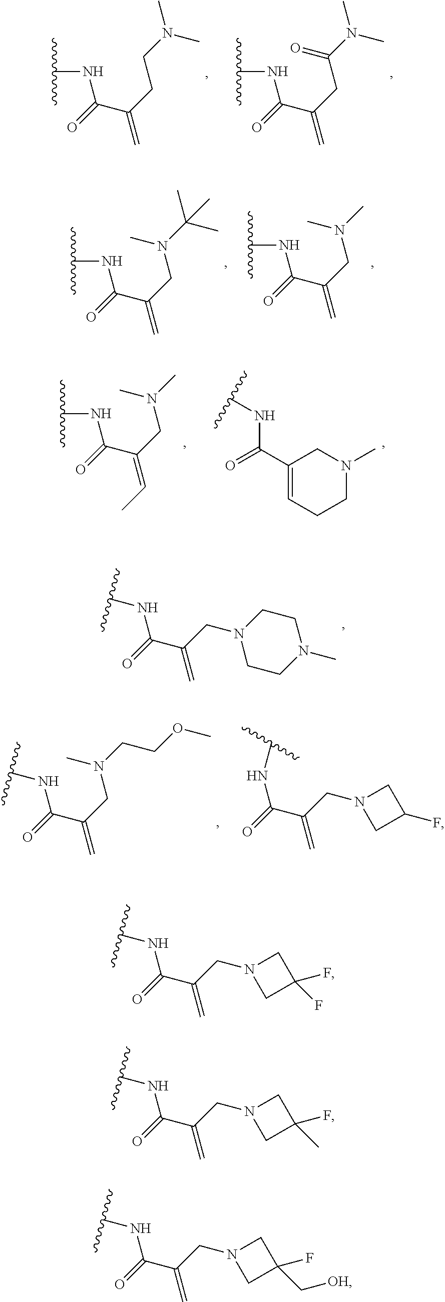

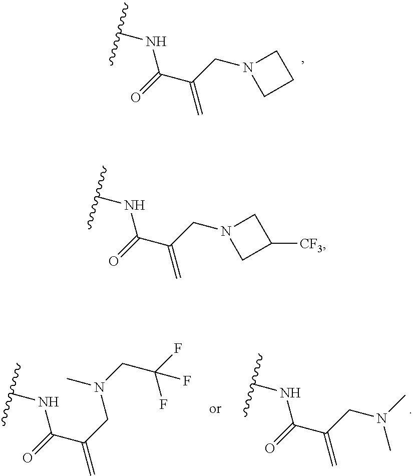

In certain embodiments, R¹ is

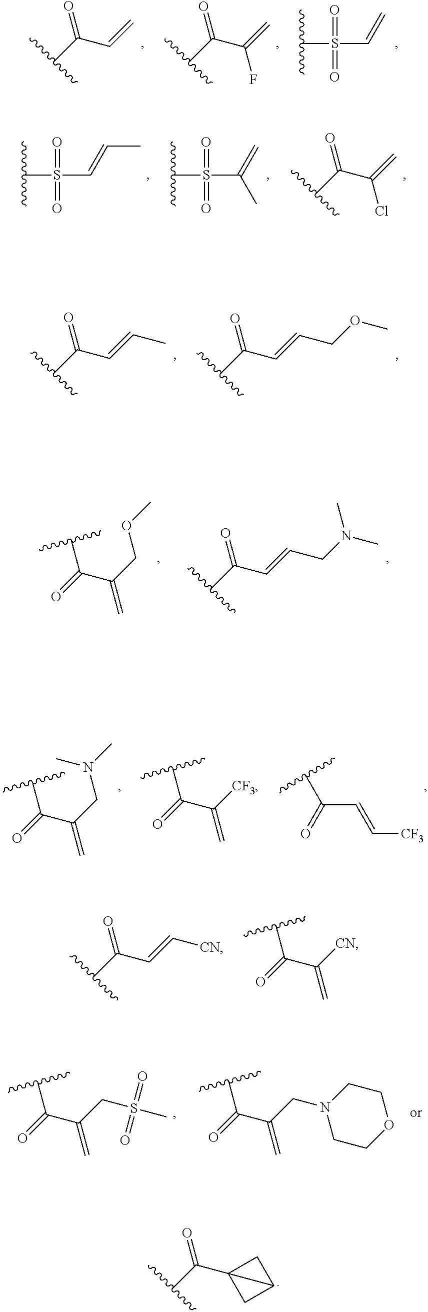

In certain embodiments, R¹ is

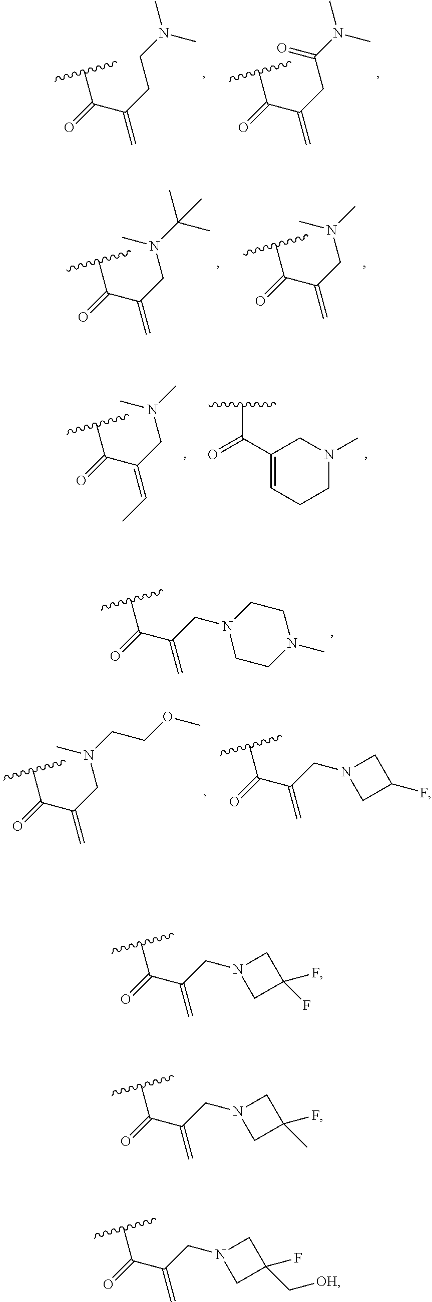

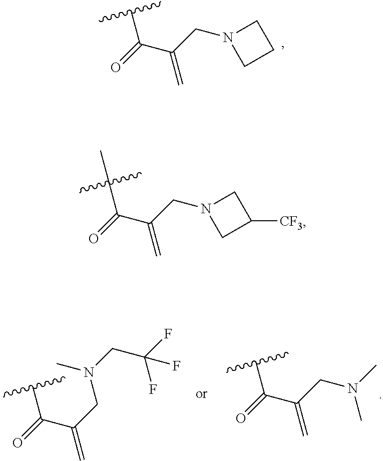

In particular embodiments, R¹ is —CH₂—C≡C—C(O)—NMe₂, or —CH₂—C(H)═C(H)—C(O)—(3,3-difluoroazetidin-1-yl).

In certain embodiments, R¹ is

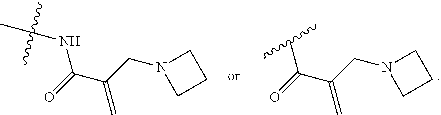

In certain embodiments, R¹ is —C(O)—C≡C—CH₂OMe, C(O)—C≡C—CH₂OH, C(O)—C≡C—C(Me)HOH.

In certain embodiments, R¹ is

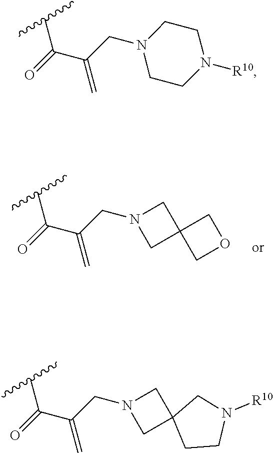

and R¹⁰ is —C(O)R¹¹ or —S(O)₂R¹¹; and R¹¹ is substituted or unsubstituted alkyl, substituted or unsubstituted aryl, or substituted or unsubstituted heteroaryl.

In certain embodiments, R¹¹ is Me, Et, i-Pr, Ph, or pyridyl,

In certain embodiments, R¹¹ is —CH₂—NH—C(O) Me, —C(Me)H—NH—C(O) Me, or —C(i-Pr)H—NH—C(O)Me.

In certain embodiments, the compound is any one of compounds listed in Table 1G. In certain embodiments, the compound is any one of compounds listed in Table 1H. In certain embodiments, the compound is any one of compounds listed in Table 1J. In certain embodiments, the compound is any one of compounds listed in Table 1K.

In certain embodiments, the compound is

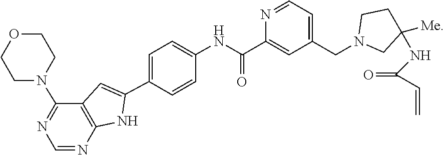 [SMILES: C=CC(=O)NC1(C)CCN(Cc2ccnc(C(=O)Nc3ccc(-c4cc5c(N6CCOCC6)ncnc5[nH]4)cc3)c2)C1]

In certain embodiments, the compound is

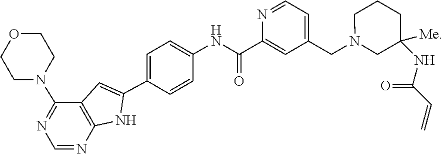 [SMILES: C=CC(=O)NC1(C)CCCN(Cc2ccnc(C(=O)Nc3ccc(-c4cc5c(N6CCOCC6)ncnc5[nH]4)cc3)c2)C1]

In certain embodiments, the compound is

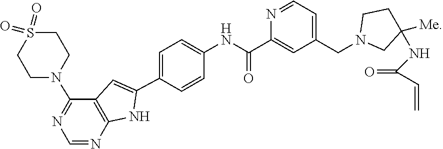 [SMILES: C=CC(=O)NC1(C)CCN(Cc2ccnc(C(=O)Nc3ccc(-c4cc5c(N6CCS(=O)(=O)CC6)ncnc5[nH]4)cc3)c2)C1]

In certain embodiments, the compound is

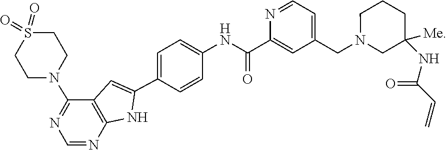 [SMILES: C=CC(=O)NC1(C)CCCN(Cc2ccnc(C(=O)Nc3ccc(-c4cc5c(N6CCS(=O)(=O)CC6)ncnc5[nH]4)cc3)c2)C1]

In certain embodiments, the compound is

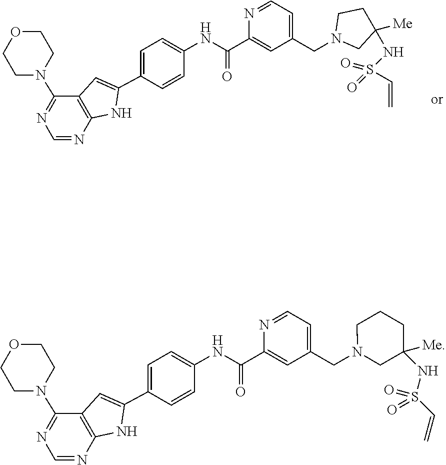 [SMILES: C=CS(=O)(=O)NC1(C)CCN(Cc2ccnc(C(=O)Nc3ccc(-c4cc5c(N6CCOCC6)ncnc5[nH]4)cc3)c2)C1 ; C=CS(=O)(=O)NC1(C)CCCN(Cc2ccnc(C(=O)Nc3ccc(-c4cc5c(N6CCOCC6)ncnc5[nH]4)cc3)c2)C1]

In certain embodiments, the compound is

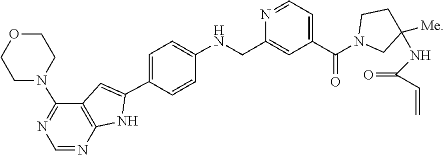 [SMILES: C=CC(=O)NC1(C)CCN(C(=O)c2ccnc(CNc3ccc(-c4cc5c(N6CCOCC6)ncnc5[nH]4)cc3)c2)C1]

In certain embodiments, the compound is

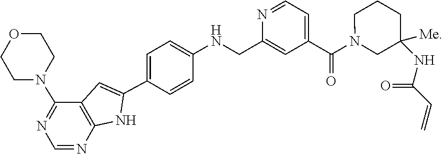 [SMILES: C=CC(=O)NC1(C)CCCN(C(=O)c2ccnc(CNc3ccc(-c4cc5c(N6CCOCC6)ncnc5[nH]4)cc3)c2)C1]

In certain embodiments, the compound is

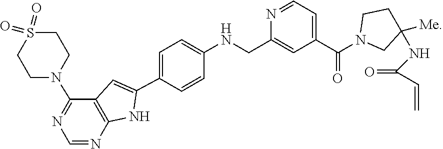 [SMILES: C=CC(=O)NC1(C)CCN(C(=O)c2ccnc(CNc3ccc(-c4cc5c(N6CCS(=O)(=O)CC6)ncnc5[nH]4)cc3)c2)C1]

In certain embodiments, the compound is

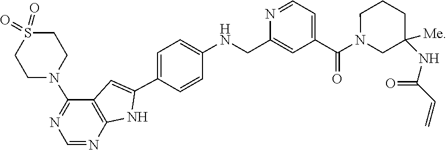 [SMILES: C=CC(=O)NC1(C)CCCN(C(=O)c2ccnc(CNc3ccc(-c4cc5c(N6CCS(=O)(=O)CC6)ncnc5[nH]4)cc3)c2)C1]

In certain embodiments, the compound is

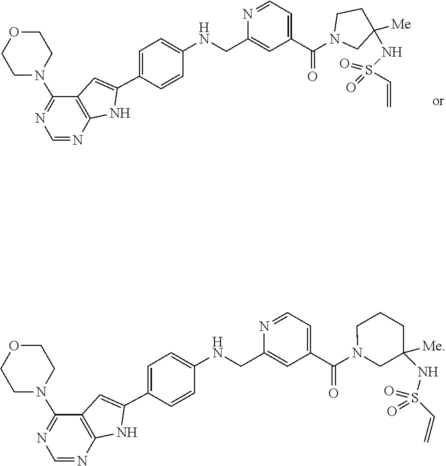 [SMILES: C=CS(=O)(=O)NC1(C)CCN(C(=O)c2ccnc(CNc3ccc(-c4cc5c(N6CCOCC6)ncnc5[nH]4)cc3)c2)C1 ; C=CS(=O)(=O)NC1(C)CCCN(C(=O)c2ccnc(CNc3ccc(-c4cc5c(N6CCOCC6)ncnc5[nH]4)cc3)c2)C1]

In certain embodiments, the compound is:

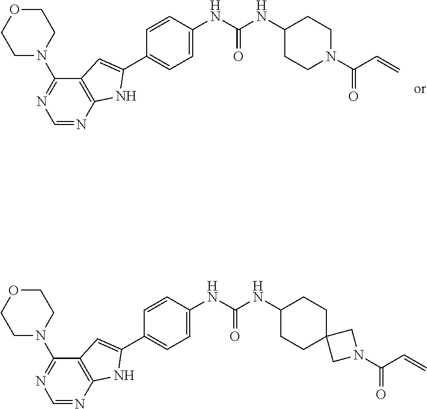 [SMILES: C=CC(=O)N1CCC(NC(=O)Nc2ccc(-c3cc4c(N5CCOCC5)ncnc4[nH]3)cc2)CC1 ; C=CC(=O)N1CC2(CCC(NC(=O)Nc3ccc(-c4cc5c(N6CCOCC6)ncnc5[nH]4)cc3)CC2)C1]

or a pharmaceutically acceptable salt thereof.

In certain embodiments, the compound is:

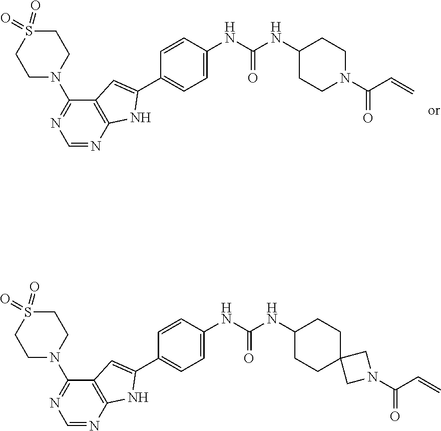 [SMILES: C=CC(=O)N1CCC(NC(=O)Nc2ccc(-c3cc4c(N5CCS(=O)(=O)CC5)ncnc4[nH]3)cc2)CC1 ; C=CC(=O)N1CC2(CCC(NC(=O)Nc3ccc(-c4cc5c(N6CCS(=O)(=O)CC6)ncnc5[nH]4)cc3)CC2)C1]

or a pharmaceutically acceptable salt thereof.

In certain embodiments, R¹ is —C(O)—C≡C—CH₂—NMe₂. In certain embodiments, R¹ is —C(O)—C≡C—CH₂-(3,3-difluoroazetidine-1-yl).

In certain embodiments, the compound is according to Formula (LX-I), or (LXI)-(LXVIII), and Cy¹ is as described herein. In certain embodiments, the compound is according to Formula (LX-I), or (LXI)-(LXVIII), and Cy¹ is

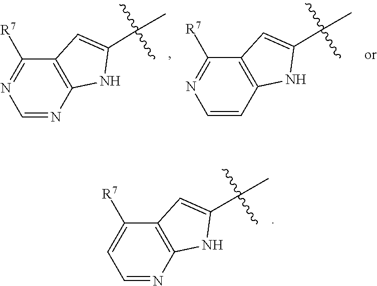

In certain embodiments, Cy¹ is

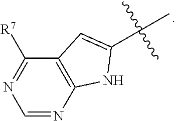

In certain embodiments, R⁷ is as described herein. In certain embodiments, the compound is according to Formula (LX-I), or (LXI)-(LXVIII), and R⁷ is

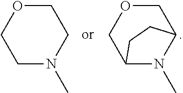

In certain embodiments, Cy² is as described herein. In certain embodiments, the compound is according to Formula (LX-I), or (LXI)-(LXVIII), and Cy² is substituted or unsubstituted Ph. In certain embodiments, the compound is according to Formula (LX-I), or (LXI)-(LXVIII), and Cy² is unsubstituted Ph.

In certain embodiments, the compound is according to Formula (LX-I), or (LXI)-(LXVIII), and Cy³ is as described herein. In certain embodiments, the compound is according to Formula (LX-I), or (LXI)-(LXVIII), and Cy³ is substituted or unsubstituted pyridylene. In certain embodiments, the compound is according to Formula (LX-I), or (LXI)-(LXVIII), and Cy³ is unsubstituted pyridylene. In certain embodiments, the compound is according to Formula (LX-I), or (LXI)-(LXVIII), and Cy³ is substituted or unsubstituted 2-pyridylene. In certain embodiments, the compound is according to Formula (LX-I), or (LXI)-(LXVIII), and Cy³ is unsubstituted 2-pyridylene.

In certain embodiments, the compound is according to Formula (LX-I), or (LXI)-(LXVIII), and L is as described herein. In certain embodiments, the compound is according to Formula (LX-I), or (LXI)-(LXVIII), and L is substituted or unsubstituted methylene. In certain embodiments, the compound is according to Formula (LX-I), or (LXI)-(LXVIII), and L is —CH₂—, CHMe-, or C(Me)₂—. In certain embodiments, the compound is according to Formula (LX-I), or (LXI)-(LXVIII), and L is —CH₂—.

In certain embodiments, the compound is according to Formula (LX-I), or (LXI)-(LXVIII), and Cy⁴ is as described herein. In certain embodiments, the compound is according to Formula (LX-I), or (LXI)-(LXVIII), and Cy⁴ is substituted or unsubstituted heterocycloalkyl. In certain embodiments, the compound is according to Formula (LX-I), or (LXI)-(LXVIII), and Cy⁴ is substituted or unsubstituted azetidinylene, pyrrolidinylene, piperidinylene, or piperazinylene, In certain embodiments, the compound is according to Formula (LX-I), or (LXI)-(LXVIII), and Cy³ is unsubstituted piperidinylene.

In certain embodiments, the compound is according to Formula (LX-I), or (LXI)-(LXVIII), and —X—W—Y- is as described herein. In certain embodiments, the compound is according to Formula (LX-I), or (LXI)-(LXVIII), and —X—W—Y—is —NH—C(H)Me-. In certain embodiments, the compound is according to Formula (LX-I), or (LXI)-(LXVIII), and —X—W—Y—is —NH—C(H)CF₃—. In certain embodiments, the compound is according to Formula (LX-I), or (LXI)-(LXVIII), and —X—W—Y—is —NH—W—Y—is —NH—C(O)—.

In certain embodiments, the compound is according to Formula (LX-I), or (LXI)-(LXVIII), and —X—

In certain embodiments, the compound is according to Formula (LX-IIa) or (LX-IIb):

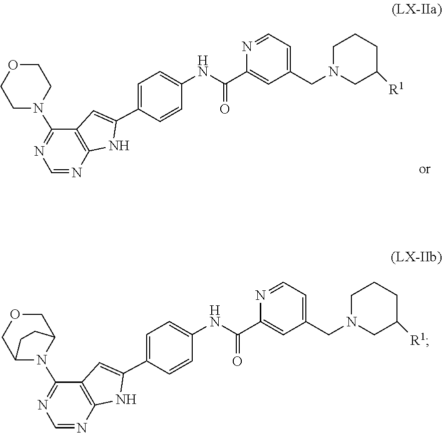

and wherein R¹ is as described above.

In certain embodiments, R¹ is as described herein. In certain embodiments, R¹ is —C(O)—CH—CH₂. In certain embodiments, R¹ is —C(O)—C≡C-Me.

In certain embodiments, R¹ is

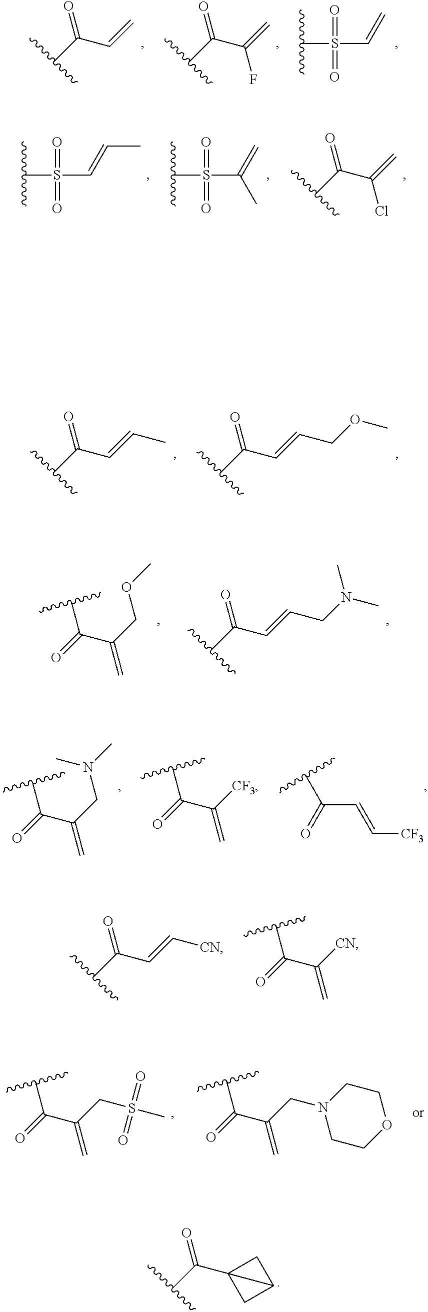

In certain embodiments, R¹ is

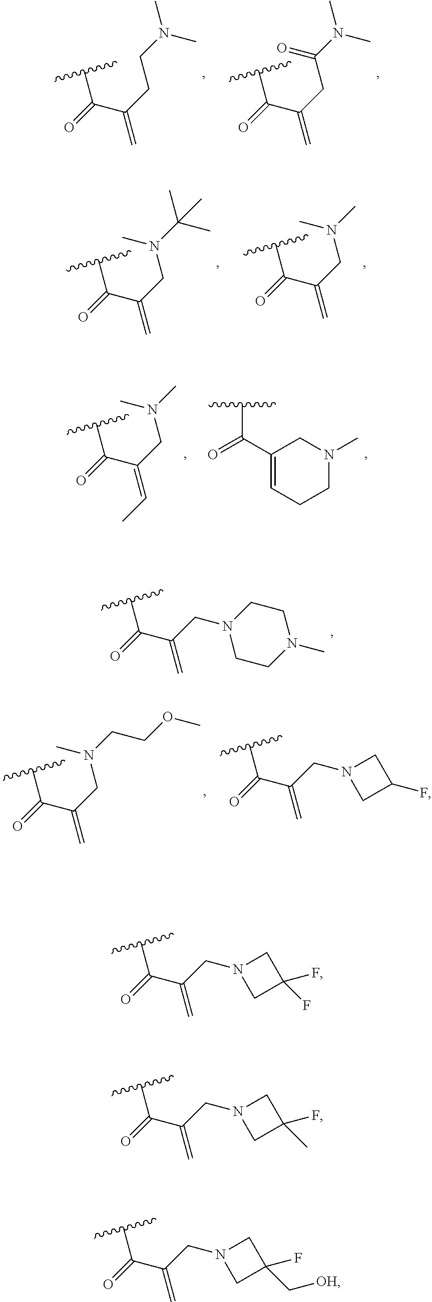

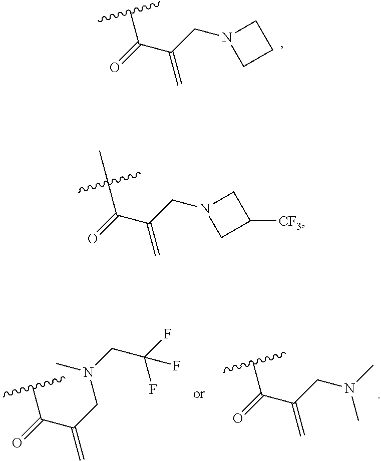

In certain embodiments, R¹ is —CH₂—C≡C—C(O)—NMe₂.

In certain embodiments, the substitution on each alkyl is 1, 2, or 3 groups independently selected from halo, CN, hydroxy, and alkoxy. In certain embodiments, the substitution on each alkoxy is 1, 2, or 3 groups independently selected from halo, CN, and substituted or unsubstituted alkyl. In certain embodiments, the substitution on each cycloalkyl is 1, 2, or 3 groups independently selected from halo, CN, hydroxy, substituted or unsubstituted alkyl, and substituted or unsubstituted alkoxy. In certain embodiments, the substitution on each heterocycloalkyl is 1, 2, or 3 groups independently selected from halo, CN, hydroxy, substituted or unsubstituted alkyl, and substituted or unsubstituted alkoxy. In certain embodiments, the substitution on each aryl is independently 1-3 groups independently selected from halo, haloalkyl, amino, dialkylamino, amido, CN, hydroxy, substituted or unsubstituted alkyl, and substituted or unsubstituted alkoxy. In certain embodiments, the substitution on each heteroaryl is independently 1-3 groups independently selected from halo, haloalkyl, amino, dialkylamino, amido, CN, hydroxy, substituted or unsubstituted alkyl, substituted or unsubstituted alkoxy, and oxo.

In certain embodiments, the compound according to formula (L-I):

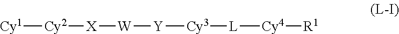

or a stereoisomer thereof; or a pharmaceutically acceptable salt thereof,
wherein:
Cy¹ is substituted or unsubstituted

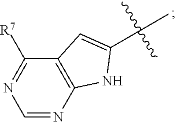

and the substitution on Cy¹ is C₁-C₄ alkyl, CN, or halo;
each of Cy² and Cy³ is independently substituted or unsubstituted cycloalkylene, substituted or unsubstituted heterocycloalkylene, substituted or unsubstituted arylene, or substituted or unsubstituted heteroarylene; Cy⁴ is absent, substituted or unsubstituted cycloalkylene, substituted or unsubstituted heterocycloalkylene, substituted or unsubstituted arylene, or substituted or unsubstituted heteroarylene; L is a single bond, or substituted or unsubstituted —CH₂—; and the substitution on CH₂ is Me, Et, OH, OMe, OEt, or O-i-Pr.R^{3a} is H or substituted or unsubstituted C₁₋₄ alkyl; and each of R^{3b}, and R^{3c} is independently H, OH, substituted or unsubstituted C₁₋₄ alkyl, substituted or unsubstituted C₁₋₄ alkoxy; R⁷ is a 4-10 membered substituted or unsubstituted heterocycloalkyl ring having 1-2 heteroatoms independently selected from nitrogen, oxygen, and sulfur, substituted or unsubstituted phenyl, an 8-10 membered substituted or unsubstituted bicyclic aryl ring, and a 5-6 membered substituted or unsubstituted heteroaryl ring having 1-4 heteroatoms independently selected from nitrogen, oxygen, and sulfur; and the substitution on the phenyl, and the bicyclic aryl is 1, 2, or 3 groups independently selected from C₁-C₄ alkyl, hydroxy, CN, C₁-C₄ alkoxy;
i) X—W—Y is —NH—, —O—, —NH—C(R^{3a}) (R^{3b})—, —S(O)₂—or —NH—C(O)—; and R¹ is —B-C(R^{6a})—C(R^{6b})—C(O)—R^{6c}, —B—C(R^{6a})═C(R^{6b})—S(O)—R^{6c}, —B—C(R^{6a})═C(R^{6b})—S(O)₂—R^{6c}, —B—C(R^{6a})—C(R^{6b})—P(O)—R^{6a}R^{6b}, or —B—C(R^{6a})═C(R^{6b})—P(O)—OR^{6a}OR^{6b}; B is substituted or unsubstituted C₁₋₄ alkylene; R^{6c} is substituted or unsubstituted alkoxy, substituted or unsubstituted amino, or substituted or unsubstituted heterocycloalkyl, having 1-2 heteroatoms independently selected from nitrogen, oxygen, and sulfur; each R^{6a} and R^{6b} is independently H, CN, halo, or substituted or unsubstituted C₁₋₆ alkyl; and the substitution on C₁₋₆ alkyl is 1, 2, or 3 groups independently selected from halo, alkoxy, alkylamino, dialkylamino, or heterocycloalkyl;
ii) X—W—Y is —NH—, —O—, —NH—C(R^{3a}) (R^{3b})—, or —S(O)₂—; and R¹ is —C(O)—C(R^{6a})═C(R^{6b})R⁶, —S(O)—C(R^{6a})═C(R^{6b})R^{6c}, —S(O)₂—C(R^{6a})═C(R^{6b})R^{6c}, —NR^{3c}—C(O)—C(R^{6a})═C(R^{6b})R^{6c}, —NR^{3c}—S(O)—C(R^{6a})═C(R^{6b})R^{6c} or —NR^{3c}—S(O)₂—C(R^{6a})═C(R^{6b})R^{6c}; and each R^{6a}, R^{6b}, and R^{6c} is independently H, CN, halo, or substituted or unsubstituted C₁₋₆ alkyl; or R^{6a} and R^{6b} are joined together to form a bond, and R^{6c} is independently H, CN, halo, or substituted or unsubstituted C₁₋₆ alkyl; and wherein at least one of R^{3a} and R^{3b} is substituted alkyl;
iii) X—W—Y is —NH—C(O)—; and R¹ is —C(O)—C(R^{6a})═C(R^{6b})R^{6c}, —S(O)—C(R^{6a})═C(R^{6b})R^{6e}, —S(O)₂—C(R^{6a})═C(R^{6b}) R^{6c}, —NR^{3c}—C(O)—C(R^{6a})—C(R^{6b})R^{6c}, —NR^{3c}—S(O)—C(R^{6a})—C(R^{6b})R^{6c}, or —NR^{3c}—S(O)₂—C(R^{6a})═C(R^{6b})R^{6c}; and R^{6a} is alkyl, substituted with alkoxy, alkylamino, dialkylamino, or substituted or unsubstituted heterocycloalkyl, and each Rob, and R^{6c} is independently H, CN, halo, or substituted or unsubstituted C₁₋₆ alkyl; or
iv) X—W—Y is —NH—C(O)—; and R¹ is —C(O)—C(R^{6a})—C(R^{6b})R^{6c}, —S(O)—C(R^{6a})═C(R^{6b})R^{6c}, —S(O)₂—C(R^{6a})═C(R^{6b}) R^{6c}, —NR^{3c}—C(O)—C(R^{6a})═C(R^{6b})R^{6c}, —NR^{3c}—S(O)—C(R^{6a})═C(R^{6b})R^{6c}, or —NR^{3c}—S(O)₂—C(R^{6a})—C(R^{6b})R^{6c}; each R^{6a}, R^{6b}, and R^{6c} is independently H, CN, halo, or substituted or unsubstituted C₁₋₆ alkyl; or R^{6a} and R^{6b} are joined together to form a bond, and R^{6c} is independently H, CN, halo, or substituted or unsubstituted C₁₋₆ alkyl; and Cy⁴ is substituted or unsubstituted heterocycloalkyl, and the heterocycloalkyl is a bridged heterocycloalkyl;
and wherein the substitution on each alkyl is 1, 2, or 3 groups independently selected from halo, CN, hydroxy, and C₁-C₄ alkoxy; the substitution on alkylene is 1, 2, or 3 groups independently selected from C₁-C₄ alkyl, CN, hydroxy, and halo; the substitution on —N(H)—is C₁-C₄ alkyl; the substitution on each alkoxy is 1, 2, or 3 groups independently selected from halo, CN, and substituted or unsubstituted C₁-C₄ alkyl; the substitution on each cycloalkyl is 1, 2, or 3 groups independently selected from halo, CN, hydroxy, substituted or unsubstituted C₁-C₄ alkyl, and substituted or unsubstituted C₁-C₄ alkoxy; the substitution on each heterocycloalkyl is 1, 2, or 3 groups independently selected from halo, CN, hydroxy, substituted or unsubstituted C₁-C₄ alkyl, and substituted or unsubstituted C₁-C₄ alkoxy; the substitution on each aryl is 1, 2, or 3 groups independently selected from halo, haloalkyl, amino, dialkylamino, amido, CN, hydroxy, substituted or unsubstituted C₁-C₄ alkyl, and substituted or unsubstituted C₁-C₄ alkoxy; the substitution on each heteroaryl is 1-3 groups independently selected from halo, haloalkyl, amino, dialkylamino, amido, CN, hydroxy, substituted or unsubstituted C₁-C₄ alkyl, substituted or unsubstituted C₁-C₄ alkoxy, and oxo; or the compound is any one of the compounds listed in Table 1G, 1H, 1J, and 1K;

In one embodiment, the compound is other than:

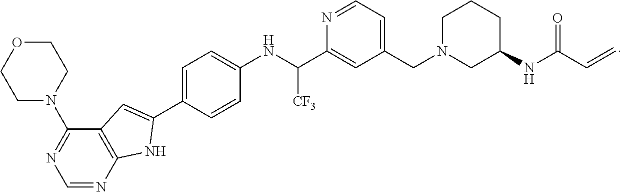 [SMILES: C=CC(=O)N[C@@H]1CCCN(Cc2ccnc(C(Nc3ccc(-c4cc5c(N6CCOCC6)ncnc5[nH]4)cc3)C(F)(F)F)c2)C1]

In certain embodiments, X—W—Y is —NH—, —O—, —NH—C(R^{3a}) (R^{3b})—, or —S(O)₂—; and R¹ is —C(O)—C(R^{6a})═C(R^{6b})R^{6c}, —S(O)—C(R^{6a})═C(R^{6b})R^{6c}, —S(O)₂—C(R^{6a})—C(R^{6b})R^{6c}, —NR^{3c}—C(O)—C(R^{6a})—C(R^{6b})R^{6c}, —NR^{3c}—S(O)—C(R^{6a})—C(R^{6b})R^{6c}, or —NR^{3c}—S(O)₂—C(R^{6a})═C(R^{6b})R^{6c}; and each R^{6a}, R^{6b}, and R^{6c} is independently H, CN, halo, or substituted or unsubstituted C₁₋₆ alkyl; or R^{6a} and R^{6b} are joined together to form a bond, and R^{6c} is independently H, CN, halo, or substituted or unsubstituted C₁₋₆ alkyl; and wherein at least one of R^{3a} and R^{3b} is substituted alkyl. In certain embodiments, —X—W—Y—is —NH—, —O—, —NH—C(R^{3a}) (R^{3b})—, or —S(O)₂—. In certain embodiments, —X—W—Y—is —NH—C(O)—. In certain embodiments, —X—W—Y—is —N(H)—or —O—. In certain embodiments, —X—W—Y—is —NH—C(R^{3a}) (R^{3b})—; and one of R^{3a} and R^{3b} is CF₃. In certain embodiments, —X—W—Y- is —N(H)—C(H)(CF₃)—. In certain embodiments, the compound is according to formula LXIa, LXIb, LXII, LXIII, LXIV, LXV, or LXVI, or a stereoisomer thereof; or a pharmaceutically acceptable salt thereof.

In certain embodiments, Cy² is substituted or unsubstituted phenylene, or substituted or unsubstituted pyridinylene; and each substitution is independently selected from 1, 2, or 3 of C₁-C₄ alkyl, hydroxy, CN, C₁-C₄ alkoxy, and halo. In certain embodiments, Cy² is unsubstituted phenylene.

In certain embodiments, Cy³ is substituted or unsubstituted phenylene, pyridylene, pyrimidinylene, pyrrolidinylene, piperidinylene, or piperazinylene; and each substitution is independently selected from 1, 2, or 3 of C₁-C₄ alkyl, amino, alkylamino, dialkylamino, hydroxy, C₁-C₄ hydroxyalkyl, CN, C₁-C₄ alkoxy, and halo.

In certain embodiments, L is a single bond. In certain embodiments, L is —CH₂—, or —C(Me) H—. In certain embodiments, Cy⁴ is absent.

In certain embodiments, Cy⁴ is substituted or unsubstituted heterocycloalkylene; and each substitution is independently selected from 1, 2, or 3 of C₁-C₄ alkyl, amino, alkylamino, dialkylamino, hydroxy, C₁-C₄ hydroxyalkyl, CN, C₁-C₄ alkoxy, and halo. In certain embodiments, Cy⁴ is substituted or unsubstituted azetidinylene, substituted or unsubstituted pyrrolidinylene, substituted or unsubstituted piperidinylene, substituted or unsubstituted piperazinylene, substituted or unsubstituted diazabicycloheptanylene, or substituted or unsubstituted diazabicyclooctanylene; and each substitution is independently selected from 1, 2, or 3 of C₁-C₄ alkyl, amino, alkylamino, dialkylamino, hydroxy, C₁-C₄ hydroxyalkyl, CN, C₁-C₄ alkoxy, and halo. In certain embodiments, Cy⁴ is unsubstituted azetidinylene, unsubstituted pyrrolidinylene, unsubstituted piperidinylene, or unsubstituted piperazinylene. In certain embodiments, Cy⁴ is azetidinylene, pyrrolidinylene, piperidinylene, or piperazinylene, each substituted with one or more alkyl, halo, or alkoxy. In certain embodiments, Cy⁴ is azetidinylene, pyrrolidinylene, piperidinylene, or piperazinylene, each substituted with one or more Me, Et, F, (F)₂, or Cl. In certain embodiments, Cy⁴ is diazabicycloheptanylene or diazabicyclooctanylene, each unsubstituted or substituted with one or more alkyl, halo, or alkoxy.

In certain embodiments, R⁷ is

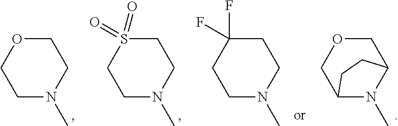

In certain embodiments, the compound is according to formula (LXVIIa) or LXVIIb), or a stereoisomer thereof; or a pharmaceutically acceptable salt thereof. In certain embodiments, the compound is according to formula (LXVIII), or a stereoisomer thereof; or a pharmaceutically acceptable salt thereof. In certain embodiments, the compound is according to formula (LXIXa), (LXIXb), or (LXIXc), or a stereoisomer thereof; or a pharmaceutically acceptable salt thereof. In certain embodiments, the compound is according to formula (LXX), or a stereoisomer thereof; or a pharmaceutically acceptable salt thereof. In certain embodiments, the compound is according to formula (LXXIa) or (LXXIb), or a stereoisomer thereof; or a pharmaceutically acceptable salt thereof. In certain embodiments, the compound is according to formula (LXXIIIa), (LXXIIIb), or (LXXIIIc), or a stereoisomer thereof; or a pharmaceutically acceptable salt thereof. In certain embodiments, the compound is according to formula (LXXIV), or a stereoisomer thereof; or a pharmaceutically acceptable salt thereof. In certain embodiments, the compound is according to formula (LXXVa) or (LXXVb), or a stereoisomer thereof; or a pharmaceutically acceptable salt thereof. In certain embodiments, the compound is according to formula (LXXVIa), (LXXVIb), (LXXVIc), (LXXVId), (LXXVIe), or (LXXVIf), or a stereoisomer thereof; or a pharmaceutically acceptable salt thereof. In certain embodiments, the compound is according to formula (LXXVIIa), (LXXVIIb), (LXXVIIc), (LXXVIId), (LXXVIIe), or (LXXVIIf), or a stereoisomer thereof; or a pharmaceutically acceptable salt thereof. In certain embodiments, the compound is according to formula (LXXVIII) or a stereoisomer thereof; or a pharmaceutically acceptable salt thereof.

In certain embodiments, R¹ is —S(O)₂—CH═CH₂, —NH—C(O)—C≡CH, —C(O)—CH═CH₂, —C(O)—C≡CH, —NH—C(O)—CH═CH₂, —C(O)—C≡C-Me, or —NH—S(O)₂—CH═CH₂. In certain embodiments, R¹ is —C(O)—CH═CH—CH₂OH, or —C(O)—CH═CH—OH. In certain embodiments, R¹ is —NH—C(O)—C(Cl)=CH₂, —NH—C(O)—C(F)=CH₂, —NH—C(O)—C(CH₂—NMe₂)=CH₂, —NH—S(O)₂—C(Me)=CH₂, —NH—C(O)—C(CH₂-morpholin-1-yl)=CH₂, or —NH—C(O)—C(CH₂—OMe)=CH₂. In certain embodiments, R¹ is —NH—C(O)—C(CH₂-morpholin-1-yl)=CH₂. In certain embodiments, R¹ is —NH—C(O)—C≡C—CH₂—NMe₂, —NH—C(O)—C(H)—C(H)—CH₂—NMe₂, —NH—C(O)—C(H)═C(H)—CH₂-(3,3-difluoroazetidin-1-yl), —NH—C(O)—C≡C-Me, —NH—C(O)—C(CH₂—SO₂Me)=CH₂, —NH—C(O)—C(CH₂—CH₂—NMe₂)=CH₂, —NH—C(O)—C(CH₂-(3,3-difluoroazetidin-1-yl))—CH₂, —NH—C(O)—C(CH₂-(3-F-3-Me-azetidin-1-yl))—CH₂, —NH—C(O)—C(CH₂—N(Me) (t-Bu))=CH₂, —NH—C(O)—C≡C—CH₂-(3,3-difluoroazetidin-1-yl), —NH—C(O)—C(CH₂-(3-F-azetidin-1-yl))=CH₂, —NH—C(O)—C(CH₂-(3-F-3-hydroxymethyl-azetidin-1-yl))=CH₂, or —NH—C(O)—C(H)═C(H)—CH₂—OMe. In certain embodiments, R¹ is —C(O)—C≡C—CH₂—NMe₂, —C(O)—C(H)═C(H)—CH₂—NMe₂, —C(O)—C(H)═C(H)—CH₂-(3,3-difluoroazetidin-1-yl), —C(O)—C≡C-Me, —C(O)—C(CH₂—SO₂Me)=CH₂, —C(O)—C(CH₂—CH₂—NMe₂)=CH₂, —C(O)—C(CH₂-(3,3-difluoroazetidin-1-yl))=CH₂, —C(O)—C(CH₂-(3-F-3-Me-azetidin-1-yl))=CH₂, —C(O)—C(CH₂—N(Me) (t-Bu))=CH₂, —C(O)—C≡C—CH₂-(3,3-difluoroazetidin-1-yl), —C(O)—C(CH₂-(3-F-azetidin-1-yl))=CH₂, —C(O)—C(CH₂-(3-F-3-hydroxymethyl-azetidin-1-yl))=CH₂, —C(O)—C(H)═C(H)—CH₂—OMe, or —C(O)—C≡C—CH₂—OMe. In certain embodiments, R¹ is —CH₂—C≡C—C(O)—NMe₂ or C(O)—C≡C—CH₂—OMe. In certain embodiments, R¹ is —CH₂—C≡C—C(O)—NMe₂, —CH₂—C≡C—C(O)-NEt₂, or —CH₂—C(H)═C(H)—C(O)—(3,3-difluoroazetidin-1-yl). In certain embodiments, R¹ is —C(O)—C≡C—CH₂OMe, C(O)—C≡C—CH₂OH, C(O)—C≡C—C(Me) HOH.

In certain embodiments, R¹ is

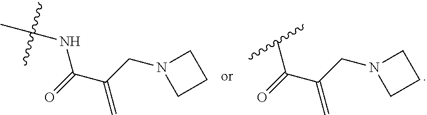

In certain embodiments, R¹ is

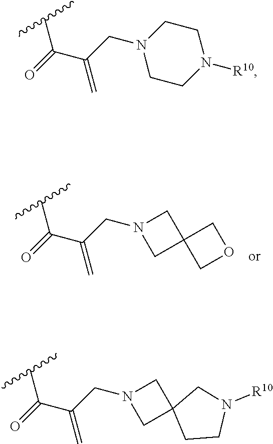

and R¹⁰ is —C(O)R¹¹ or —S(O)₂R¹¹; and R¹¹ is substituted or unsubstituted alkyl, substituted or unsubstituted aryl, or substituted or unsubstituted heteroaryl. In certain embodiments, R¹¹ is Me, Et, i-Pr, Ph, or pyridyl. In certain embodiments, R¹¹ is —CH₂—NH—C(O) Me, —C(Mc) H—NH—C(O)Me, or —C(i-Pr) H—NH—C(O)Mc.

In a particular embodiment, the compound is Compound ID 1102:

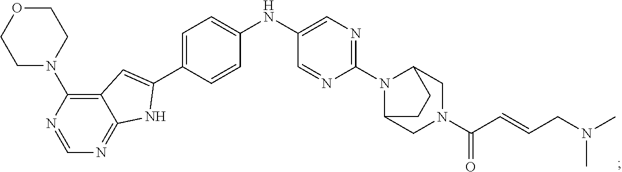 [SMILES: CN(C)C/C=C/C(=O)N1CC2CCC(C1)N2c1ncc(Nc2ccc(-c3cc4c(N5CCOCC5)ncnc4[nH]3)cc2)cn1]

or a stereoisomer thereof; or a pharmaceutically acceptable salt thereof.

In certain embodiments, the compound is any one of compounds listed below:

| ID | Name |
|---|---|
| 310 | 1-[4-hydroxy-4-[[4-(4-morpholino-7H-pyrrolo[2,3-d]pyrimidin-6-yl)anilino]methyl]-1- |
| | piperidyl]prop-2-en-1-one |
| 314 | 1-[4-[2,2,2-trifluoro-1-[4-(4-morpholino-7H-pyrrolo[2,3-d]pyrimidin-6-yl)anilino]ethyl]-1- |
| | piperidyl]prop-2-en-1-one |
| 401 | N-[4-[4-(4,4-difluoro-1-piperidyl)-7H-pyrrolo[2,3-d]pyrimidin-6-yl]phenyl]-4-[[(3R)-3-(prop-2- |
| | enoylamino)-1-piperidyl]methyl]pyridine-2-carboxamide |
| 402 | N-[4-[4-(1,1-dioxo-1,4-thiazinan-4-yl)-7H-pyrrolo[2,3-d]pyrimidin-6-yl]phenyl]-4-[[(3R)-3- |
| | (prop-2-enoylamino)-1-piperidyl]methyl]pyridine-2-carboxamide |
| 407 | 4-[[(3R)-3-methyl-3-(prop-2-enoylamino)-1-piperidyl]methyl]-N-[4-(4-morpholino-7H- |
| | pyrrolo[2,3-d]pyrimidin-6-yl)phenyl]pyridine-2-carboxamide |
| 419 | N-[4-(4-morpholino-7H-pyrrolo[2,3-d]pyrimidin-6-yl)phenyl]-2-[[(3R)-3-(prop-2-enoylamino)- |
| | 1-piperidyl]methyl]pyridine-4-carboxamide |
| 429 | N-[4-[4-(3-oxa-8-azabicyclo[3.2.1]octan-8-yl)-7H-pyrrolo[2,3-d]pyrimidin-6-yl]phenyl]-4- |
| | [[(3R)-3-(prop-2-enoylamino)-1-piperidyl]methyl]pyridine-2-carboxamide |
| 451 | 4-└└(3R)-3-(2-chloroprop-2-enoylamino)-1-piperidyl┘methyl┘-N-└4-(4-morpholino-7H- |
| | pyrrolo[2,3-d]pyrimidin-6-yl)phenyl]pyridine-2-carboxamide |
| 601 | 4-[[(3R)-3-[4-(dimethylamino)but-2-ynoylamino]-1-piperidyl]methyl]-N-[4-(4-morpholino-7H- |
| | pyrrolo[2,3-d]pyrimidin-6-yl)phenyl]pyridine-2-carboxamide |
| 602 | 4-[[(3R)-3-[[(E)-4-(dimethylamino)but-2-enoyl]amino]-3-methyl-1-piperidyl]methyl]-N-[4-(4- |
| | morpholino-7H-pyrrolo[2,3-d]pyrimidin-6-yl)phenyl]pyridine-2-carboxamide |
| 603 | 4-[[(3R)-3-[[(E)-4-(3,3-difluoroazetidin-1-yl)but-2-enoyl]amino]-3-methyl-1-piperidyl]methyl]- |
| | N-[4-(4-morpholino-7H-pyrrolo[2,3-d]pyrimidin-6-yl)phenyl]pyridine-2-carboxamide |
| 604 | 4-[[(3R)-3-[4-(dimethylamino)but-2-ynoylamino]-3-methyl-1-piperidyl]methyl]-N-[4-(4- |
| | morpholino-7H-pyrrolo[2,3-d]pyrimidin-6-yl)phenyl]pyridine-2-carboxamide |
| 605 | 4-[[(3R)-3-methyl-3-(prop-2-enoylamino)-1-piperidyl]methyl]-N-[4-[4-(3-oxa-8- |
| | azabicyclo[3.2.1]octan-8-yl)-7H-pyrrolo[2,3-d]pyrimidin-6-yl]phenyl]pyridine-2-carboxamide |
| 606 | N-[4-(4-morpholino-7H-pyrrolo[2,3-d]pyrimidin-6-yl)phenyl]-5-prop-2-enoyl-3,3a,4,6,7,7a- |
| | hexahydro-1H-pyrrolo[3,4-c]pyridine-2-carboxamide |
| 607 | 1-[1-[(E)-4-(dimethylamino)but-2-enoyl]-4-piperidyl]-N-[4-[4-(3-oxa-8-azabicyclo[3.2.1]octan- |
| | 8-yl)-7H-pyrrolo[2,3-d]pyrimidin-6-yl]phenyl]piperidine-4-carboxamide |
| 608 | N-[5-(4-morpholino-7H-pyrrolo[2,3-d]pyrimidin-6-yl)pyrimidin-2-yl]-4-[(3R)-3-(prop-2- |
| | enoylamino)-1-piperidyl]methyl]pyridine-2-carboxamide |
| 609 | N-[4-[4-(3-oxa-8-azabicyclo[3.2.1]octan-8-yl)-7H-pyrrolo[2,3-d]pyrimidin-6-yl]phenyl]-1-(1- |
| | prop-2-enoyl-4-piperidyl)piperidine-4-carboxamide |
| 610 | 1-(1-but-2-ynoyl-4-piperidyl)-N-[4-[4-(3-oxa-8-azabicyclo[3.2.1]octan-8-yl)-7H-pyrrolo[2,3- |
| | d]pyrimidin-6-yl]phenyl]piperidine-4-carboxamide |
| 611 | 4-[[(3R)-3-[2-(methylsulfonylmethyl)prop-2-enoylamino]-1-piperidyl]methyl]-N-[4-(4- |
| | morpholino-7H-pyrrolo[2,3-d]pyrimidin-6-yl)phenyl]pyridine-2-carboxamide |
| 612 | 4-[[(3R)-3-[[4-(dimethylamino)-2-methylene-butanoyl]amino]-1-piperidyl]methyl]-N-[4-(4- |
| | morpholino-7H-pyrrolo[2,3-d]pyrimidin-6-yl)phenyl]pyridine-2-carboxamide |
| 613 | 4-[[(3R)-3-[2-[(3,3-difluoroazetidin-1-yl)methyl]prop-2-enoylamino]-1-piperidyl]methyl]-N-[4- |
| | (4-morpholino-7H-pyrrolo[2,3-d]pyrimidin-6-yl)phenyl]pyridine-2-carboxamide |
| 614 | 4-[[(3R)-3-[2-[(3-fluoro-3-methyl-azetidin-1-yl)methyl]prop-2-enoylamino]-1- |
| | piperidyl]methyl]-N-[4-(4-morpholino-7H-pyrrolo[2,3-d]pyrimidin-6-yl)phenyl]pyridine-2- |
| | carboxamide |
| 615 | 4-[[(3R)-3-[2-[[tert-butyl(methyl)amino]methyl]prop-2-enoylamino]-1-piperidyl]methyl]-N-[4- |
| | (4-morpholino-7H-pyrrolo[2,3-d]pyrimidin-6-yl)phenyl]pyridine-2-carboxamide |
| 616 | 1-[4-[4-[2,2,2-trifluoro-1-[4-(4-morpholino-7H-pyrrolo[2,3-d]pyrimidin-6-yl)anilino]ethyl]-1- |
| | piperidyl]-1-piperidyl]prop-2-en-1-one |
| 617 | 2-fluoro-N-[4-[4-(3-oxa-8-azabicyclo[3.2.1]octan-8-yl)-7H-pyrrolo[2,3-d]pyrimidin-6- |
| | yl]phenyl]-5-[[(3R)-3-(prop-2-enoylamino)-1-piperidyl]methyl]benzamide |
| 618 | 4-[[(3R)-3-[4-(3,3-difluoroazetidin-1-yl)but-2-ynoylamino]-1-piperidyl]methyl]-N-[4-(4- |
| | morpholino-7H-pyrrolo[2,3-d]pyrimidin-6-yl)phenyl]pyridine-2-carboxamide |
| 619 | 1-[4-[4-[[4-(4-morpholino-7H-pyrrolo[2,3-d]pyrimidin-6-yl)anilino]methyl]-1-piperidyl]-1- |
| | piperidyl]prop-2-en-1-one |
| 620 | (E)-1-(3,3-difluoroazetidin-1-yl)-4-[4-hydroxy-4-[[4-[4-(3-oxa-8-azabicyclo[3.2.1]octan-8-yl)- |
| | 7H-pyrrolo[2,3-d]pyrimidin-6-yl]anilino]methyl]-1-piperidyl]but-2-en-1-one |
| 621 | 4-[[(3R)-3-[2-[(3-fluoroazetidin-1-yl)methyl]prop-2-enoylamino]-1-piperidyl]methyl]-N-[4-(4- |
| | morpholino-7H-pyrrolo[2,3-d]pyrimidin-6-yl)phenyl]pyridine-2-carboxamide |
| 622 | N-[4-(4-morpholino-7H-pyrrolo[2,3-d]pyrimidin-6-yl)phenyl]-4-[[3-(prop-2- |
| | enoylamino)azetidin-1-yl]methyl]pyridine-2-carboxamide |
| 623 | N-[(3R)-1-[5-[4-(4-morpholino-7H-pyrrolo[2,3-d]pyrimidin-6-yl)anilino]pyrimidin-2-yl]-3- |
| | piperidyl]prop-2-enamide |
| 624 | 1-[4-fluoro-4-[[4-[4-(3-oxa-8-azabicyclo[3.2.1]octan-8-yl)-7H-pyrrolo[2,3-d]pyrimidin-6- |
| | yl]anilino]methyl]-1-piperidyl]prop-2-en-1-one |
| 625 | N-[(3R)-1-[[2-[1-[4-[4-(3-oxa-8-azabicyclo[3.2.1]octan-8-yl)-7H-pyrrolo[2,3-d]pyrimidin-6- |
| | yl┐anilino┐ethyl┐-4-pyridyl┐methyl┐-3-piperidyl┐prop-2-enamide |
| 626 | 1-[4-[(1R)-2,2,2-trifluoro-1-[4-(4-morpholino-7H-pyrrolo[2,3-d]pyrimidin-6-yl)anilino]ethyl]-1- |
| | piperidyl]prop-2-en-1-one |
| 627 | 1-[4-[(1S)-2,2,2-trifluoro-1-[4-(4-morpholino-7H-pyrrolo[2,3-d]pyrimidin-6-yl)anilino]ethyl]-1- |
| | piperidyl]prop-2-en-1-one |
| 628 | 4-[[(3R)-3-[2-[[3-fluoro-3-(hydroxymethyl)azetidin-1-yl]methyl]prop-2-enoylamino]-1- |
| | piperidyl]methyl]-N-[4-(4-morpholino-7H-pyrrolo[2,3-d]pyrimidin-6-yl)phenyl]pyridine-2- |
| | carboxamide |
| 629 | N-[(3R)-1-[[6-amino-5-[[4-(4-morpholino-7H-pyrrolo[2,3-d]pyrimidin-6-yl)phenoxy]methyl]-3- |
| | pyridyl]methyl]-3-piperidyl]prop-2-enamide |
| 701 | N,N-dimethyl-4-[4-[[4-(4-morpholino-7H-pyrrolo[2,3-d]pyrimidin-6-yl)anilino]methyl]-1- |
| | piperidyl]but-2-ynamide |
| 702 | 4-[4-hydroxy-4-[[4-[4-(3-oxa-8-azabicyclo[3.2.1]octan-8-yl)-7H-pyrrolo[2,3-d]pyrimidin-6- |
| | yl┘anilino┘methyl┘-1-piperidyl┘-N,N-dimethyl-but-2-ynamide |
| 703 | N,N-dimethyl-4-[4-[2,2,2-trifluoro-1-[4-[4-(3-oxa-8-azabicyclo[3.2.1]octan-8-yl)-7H- |
| | pyrrolo[2,3-d]pyrimidin-6-yl]anilino]ethyl]-1-piperidyl]but-2-ynamide |
| 704 | N,N-dimethyl-4-[4-[(1R)-2,2,2-trifluoro-1-[4-(4-morpholino-7H-pyrrolo[2,3-d]pyrimidin-6- |
| | yl)anilino]ethyl]-1-piperidyl]but-2-ynamide |
| 705 | N,N-dimethyl-4-[4-[(1S)-2,2,2-trifluoro-1-[4-(4-morpholino-7H-pyrrolo[2,3-d]pyrimidin-6- |
| | yl)anilino]ethyl]-1-piperidyl]but-2-ynamide |
| 706 | N,N-dimethyl-4-[3-[4-(4-morpholino-7H-pyrrolo[2,3-d]pyrimidin-6-yl)phenyl]-2,4-dioxo-1,3,7- |
| | triazaspiro[4.4]nonan-7-yl]but-2-ynamide |
| 801 | N-[[4-[[(3R)-3-(1,1-dioxo-1,2-thiazolidin-2-yl)-1-piperidyl]methyl]-2-pyridyl]methyl]-4-(4- |
| | morpholino-7H-pyrrolo[2,3-d]pyrimidin-6-yl)aniline |
| 901 | 4-[[(3R)-3-(2-fluoroprop-2-enoylamino)-1-piperidyl]methyl]-N-[4-[4-(3-oxa-8- |
| | azabicyclo[3.2.1]octan-8-yl)-7H-pyrrolo[2,3-d]pyrimidin-6-yl]phenyl]pyridine-2-carboxamide |
| 902 | N-[4-(4-morpholino-7H-pyrrolo[2,3-d]pyrimidin-6-yl)phenyl]-4-[[(3R)-3-[2-[[3- |
| | (trifluoromethyl)azetidin-1-yl]methyl]prop-2-enoylamino]-1-piperidyl]methyl]pyridine-2- |
| | carboxamide |
| 903 | N-[4-[4-(3-oxa-8-azabicyclo[3.2.1]octan-8-yl)-7H-pyrrolo[2,3-d]pyrimidin-6-yl]phenyl]-4-[[3- |
| | (prop-2-enoylamino)azetidin-1-yl]methyl]pyridine-2-carboxamide |
| 904 | 2-(azetidin-1-ylmethyl)-N-[(3R)-1-[[2-[(1R)-1-[4-[4-(3-oxa-8-azabicyclo[3.2.1]octan-8-yl)-7H- |
| | pyrrolo[2,3-d]pyrimidin-6-yl]anilino]ethyl]-4-pyridyl]methyl]-3-piperidyl]prop-2-enamide |
| 905 | 2-(azetidin-1-ylmethyl)-N-[(3R)-1-[[2-[(1S)-1-[4-[4-(3-oxa-8-azabicyclo[3.2.1]octan-8-yl)-7H- |
| | pyrrolo[2,3-d]pyrimidin-6-yl]anilino]ethyl]-4-pyridyl]methyl]-3-piperidyl]prop-2-enamide |
| 906 | 1-[4-[5-[4-(4-morpholino-7H-pyrrolo[2,3-d]pyrimidin-6-yl)anilino]pyrimidin-2-yl]piperazin-1- |
| | yl]prop-2-en-1-one |
| 907 | (E)-1-(3,3-difluoroazetidin-1-yl)-4-[4-[5-[4-(4-morpholino-7H-pyrrolo[2,3-d]pyrimidin-6- |
| | yl)anilino]pyrimidin-2-yl]piperazin-1-yl]but-2-en-1-one |
| 908 | 1-[4-[4-hydroxy-4-[[4-(4-morpholino-7H-pyrrolo[2,3-d]pyrimidin-6-yl)anilino]methyl]-1- |
| | piperidyl]-1-piperidyl]prop-2-en-1-one |
| 909 | 1-[3-[4-[2,2,2-trifluoro-1-[4-(4-morpholino-7H-pyrrolo[2,3-d]pyrimidin-6-yl)anilino]ethyl]-1- |
| | piperidyl]azetidin-1-yl]prop-2-en-1-one |
| 910 | N-[1-[5-[4-(4-morpholino-7H-pyrrolo[2,3-d]pyrimidin-6-yl)anilino]pyrimidin-2-yl]-4- |
| | piperidyl]prop-2-enamide |
| 911 | N-[(3S)-1-[5-[4-(4-morpholino-7H-pyrrolo[2,3-d]pyrimidin-6-yl)anilino]pyrimidin-2- |
| | yl]pyrrolidin-3-yl]prop-2-enamide |
| 912 | N-[(3S)-1-[5-[4-(4-morpholino-7H-pyrrolo[2,3-d]pyrimidin-6-yl)anilino]pyrimidin-2-yl]-3- |
| | piperidyl]prop-2-enamide |
| 913 | N-[1-[5-[4-(4-morpholino-7H-pyrrolo[2,3-d]pyrimidin-6-yl)anilino]pyrimidin-2-yl]azetidin-3- |
| | yl]prop-2-enamide |
| 914 | N-[(3R)-1-[4-methyl-5-[4-(4-morpholino-7H-pyrrolo[2,3-d]pyrimidin-6-yl)anilino]pyrimidin-2- |
| | yl]-3-piperidyl]prop-2-enamide |
| 915 | 2-(azetidin-1-ylmethyl)-N-[(3R)-1-[5-[4-(4-morpholino-7H-pyrrolo[2,3-d]pyrimidin-6- |
| | yl)anilino]pyrimidin-2-yl]-3-piperidyl]prop-2-enamide |
| 916 | 4-[[(3R)-3-[2-(azetidin-1-ylmethyl)prop-2-enoylamino]-1-piperidyl]methyl]-N-[4-(4- |
| | morpholino-7H-pyrrolo[2,3-d]pyrimidin-6-yl)phenyl]pyridine-2-carboxamide |
| 917 | N-[5-(4-morpholino-7H-pyrrolo[2,3-d]pyrimidin-6-yl)pyrazin-2-yl]-4-[[(3R)-3-(prop-2- |
| | enoylamino)-1-piperidyl]methyl]pyridine-2-carboxamide |
| 918 | N-[2-(4-morpholino-7H-pyrrolo[2,3-d]pyrimidin-6-yl)pyrimidin-5-yl]-4-[[(3R)-3-(prop-2- |
| | enoylamino)-1-piperidyl]methyl]pyridine-2-carboxamide |
| 919 | 2-┌(3,3-difluoroazetidin-1-yl)methyl┐-1-┌4-┌2,2,2-trifluoro-1-┌4-┌4-(3-oxa-8- |
| | azabicyclo[3.2.1]octan-8-yl)-7H-pyrrolo[2,3-d]pyrimidin-6-yl]anilino]ethyl]-1-piperidyl]prop-2- |
| | en-1-one |
| 920 | N-[(3R)-1-[5-[4-(4-morpholino-7H-pyrrolo[2,3-d]pyrimidin-6-yl)anilino]pyrimidin-2- |
| | yl]pyrrolidin-3-yl]prop-2-enamide |
| 921 | N-[(3R)-1-[5-[4-[4-(3-oxa-8-azabicyclo[3.2.1]octan-8-yl)-7H-pyrrolo[2,3-d]pyrimidin-6- |
| | yl]anilino]pyrimidin-2-yl]-3-piperidyl]prop-2-enamide |
| 922 | N-[(3R)-1-[5-[2-methyl-4-(4-morpholino-7H-pyrrolo[2,3-d]pyrimidin-6-yl)anilino]pyrimidin-2- |
| | yl]-3-piperidyl]prop-2-enamide |
| 923 | N-[(3R)-1-[[2-[5-[4-[4-(3-oxa-8-azabicyclo[3.2.1]octan-8-yl)-7H-pyrrolo[2,3-d]pyrimidin-6- |
| | yl┐phenyl┐-1H-imidazol-2-yl┐-4-pyridyl┐methyl┐-3-piperidyl┐prop-2-enamide |
| 924 | 2-(azetidin-1-ylmethyl)-1-[4-[4-[2,2,2-trifluoro-1-[4-(4-morpholino-7H-pyrrolo[2,3-d]pyrimidin- |
| | 6-yl)anilino]ethyl]-1-piperidyl]-1-piperidyl]prop-2-en-1-one |
| 925 | 1-[4-[(1R)-2,2,2-trifluoro-1-[4-[4-(3-oxa-8-azabicyclo[3.2.1]octan-8-yl)-7H-pyrrolo[2,3- |
| | d]pyrimidin-6-yl]anilino]ethyl]-1-piperidyl]prop-2-en-1-one |
| 926 | 1-[4-[(1S)-2,2,2-trifluoro-1-[4-[4-(3-oxa-8-azabicyclo[3.2.1]octan-8-yl)-7H-pyrrolo[2,3- |
| | d]pyrimidin-6-yl]anilino]ethyl]-1-piperidyl]prop-2-en-1-one |
| 927 | 1-[4-[2,2,2-trifluoro-1-[4-[4-(3-oxa-8-azabicyclo[3.2.1]octan-8-yl)-7H-pyrrolo[2,3-d]pyrimidin- |
| | 6-yl]anilino]ethyl]-1-piperidyl]prop-2-en-1-one |
| 928 | N-[(3R)-1-[5-[4-(4-morpholino-7H-pyrrolo[2,3-d]pyrimidin-6-yl)anilino]-2-pyridyl]-3- |
| | piperidyl]prop-2-enamide |
| 929 | 1-[4-[5-[4-[4-(3-oxa-8-azabicyclo[3.2.1]octan-8-yl)-7H-pyrrolo[2,3-d]pyrimidin-6- |
| | yl]anilino]pyrimidin-2-yl]piperazin-1-yl]prop-2-en-1-one |
| 930 | (E)-4-(dimethylamino)-1-[4-[5-[4-(4-morpholino-7H-pyrrolo[2,3-d]pyrimidin-6- |
| | yl)anilino]pyrimidin-2-yl]piperazin-1-yl]but-2-en-1-one |
| 931 | (E)-4-(azetidin-1-yl)-1-[4-[5-[4-(4-morpholino-7H-pyrrolo[2,3-d]pyrimidin-6- |
| | yl)anilino]pyrimidin-2-yl]piperazin-1-yl]but-2-en-1-one |
| 932 | 1-[4-[5-[4-(4-morpholino-7H-pyrrolo[2,3-d]pyrimidin-6-yl)anilino]-2-pyridyl]piperazin-1- |
| | yl]prop-2-en-1-one |
| 933 | N-[(3R)-1-[5-[4-(4-morpholino-7H-pyrrolo[2,3-d]pyrimidin-6-yl)anilino]pyrazin-2-yl]-3- |
| | piperidyl]prop-2-enamide |
| 934 | N-[4-(4-morpholino-7H-pyrrolo[2,3-d]pyrimidin-6-yl)phenyl]-4-[[3-[(E)-prop-1-enyl]sulfonyl- |
| | 1-piperidyl]methyl]pyridine-2-carboxamide |
| 935 | 1-[4-[5-[4-(4-morpholino-7H-pyrrolo[2,3-d]pyrimidin-6-yl)anilino]pyrazin-2-yl]piperazin-1- |
| | yl]prop-2-en-1-one |
| 936 | 1-[4-[3-[2,2,2-trifluoro-1-[4-(4-morpholino-7H-pyrrolo[2,3-d]pyrimidin-6- |
| | yl)anilino┘ethyl┘azetidin-1-yl┘-1-piperidyl┘prop-2-en-1-one |
| 937 | 1-[4-[5-[4-(4-morpholino-1H-pyrrolo[3,2-c]pyridin-2-yl)anilino]pyrimidin-2-yl]piperazin-1- |
| | yl]prop-2-en-1-one |
| 938 | 1-[4-[2-[4-(4-morpholino-7H-pyrrolo[2,3-d]pyrimidin-6-yl)anilino]pyrimidin-5-yl]piperazin-1- |
| | yl]prop-2-en-1-one |
| 939 | 1-[3-[4-[(1R)-2,2,2-trifluoro-1-[4-(4-morpholino-7H-pyrrolo[2,3-d]pyrimidin-6- |
| | yl)anilino]ethyl]-1-piperidyl]pyrrolidin-1-yl]prop-2-en-1-one |
| 940 | 1-[4-[4-[2,2,2-trifluoro-1-[4-(4-morpholino-1H-pyrrolo[3,2-c]pyridin-2-yl)anilino]ethyl]-1- |
| | piperidyl]-1-piperidyl]prop-2-en-1-one |
| 941 | 1-[3-[4-[(1S)-2,2,2-trifluoro-1-[4-(4-morpholino-7H-pyrrolo[2,3-d]pyrimidin-6-yl)anilino]ethyl]- |
| | 1-piperidyl]pyrrolidin-1-yl]prop-2-en-1-one |
| 942 | 1-[(3S)-3-[(1R)-2,2,2-trifluoro-1-[4-(4-morpholino-7H-pyrrolo[2,3-d]pyrimidin-6- |
| | yl)anilino]ethyl]-1-piperidyl]prop-2-en-1-one |
| 943 | 1-[(3R)-3-[(1R)-2,2,2-trifluoro-1-[4-(4-morpholino-7H-pyrrolo[2,3-d]pyrimidin-6- |
| | yl)anilino]ethyl]-1-piperidyl]prop-2-en-1-one |
| 944 | 1-[4-[2,2,2-trifluoro-1-[4-(4-morpholino-1H-pyrrolo[3,2-c]pyridin-2-yl)anilino]ethyl]-1- |
| | piperidyl]prop-2-en-1-one |
| 945 | 1-[4-[6-[4-(4-morpholino-7H-pyrrolo[2,3-d]pyrimidin-6-yl)anilino]pyridazin-3-yl]piperazin-1- |
| | yl]prop-2-en-1-one |
| 946 | 1-[(3S)-3-[(1S)-2,2,2-trifluoro-1-[4-(4-morpholino-7H-pyrrolo[2,3-d]pyrimidin-6- |
| | yl)anilino]ethyl]-1-piperidyl]prop-2-en-1-one |
| 947 | 1-[(3R)-3-[(1S)-2,2,2-trifluoro-1-[4-(4-morpholino-7H-pyrrolo[2,3-d]pyrimidin-6- |
| | yl)anilino]ethyl]-1-piperidyl]prop-2-en-1-one |
| 948 | 1-[4-[(3S)-3-[(1S)-2,2,2-trifluoro-1-[4-(4-morpholino-7H-pyrrolo[2,3-d]pyrimidin-6- |
| | yl)anilino]ethyl]pyrrolidin-1-yl]-1-piperidyl]prop-2-en-1-one |
| 949 | 1-[4-[(1R)-2,2,2-trifluoro-1-[4-(4-morpholino-1H-pyrrolo[3,2-c]pyridin-2-yl)anilino]ethyl]-1- |
| | piperidyl]prop-2-en-1-one |
| 950 | 1-[4-[(1S)-2,2,2-trifluoro-1-[4-(4-morpholino-1H-pyrrolo[3,2-c]pyridin-2-yl)anilino]ethyl]-1- |
| | piperidyl]prop-2-en-1-one |
| 951 | 4-hydroxy-1-[4-[5-[4-(4-morpholino-7H-pyrrolo[2,3-d]pyrimidin-6-yl)anilino]pyrimidin-2- |
| | yl]piperazin-1-yl]but-2-yn-1-one |
| 952 | 1-[(3S)-3-[(1R)-2,2,2-trifluoro-1-[4-(4-morpholino-7H-pyrrolo[2,3-d]pyrimidin-6- |
| | yl)anilino]ethyl]pyrrolidin-1-yl]prop-2-en-1-one |
| 953 | N-[(3R)-1-[[2-[2-[4-[4-(3-oxa-8-azabicyclo[3.2.1]octan-8-yl)-7H-pyrrolo[2,3-d]pyrimidin-6- |
| | yl\|phenyl\|-1H-imidazol-5-yl]-4-pyridyl\|methyl\|-3-piperidyl\|prop-2-enamide |
| 954 | N-[(3R)-1-[6-[4-(4-morpholino-7H-pyrrolo[2,3-d]pyrimidin-6-yl)anilino]pyridazin-3-yl]-3- |
| | piperidyl]prop-2-enamide |
| 955 | 1-[4-[5-[4-(4-morpholino-1H-imidazo[4,5-c]pyridin-2-yl)anilino]pyrimidin-2-yl]piperazin-1- |
| | yl]prop-2-en-1-one |
| 956 | (E)-4-(dimethylamino)-1-[4-[5-[4-(4-morpholino-1H-pyrrolo[3,2-c]pyridin-2- |
| | yl)anilino]pyrimidin-2-yl]piperazin-1-yl]but-2-en-1-one |
| 957 | N-methyl-N-[(3R)-1-[[5-[4-(4-morpholino-7H-pyrrolo[2,3-d]pyrimidin-6-yl)anilino]pyrimidin- |
| | 2-yl]methyl]-3-piperidyl]prop-2-enamide |
| 958 | 1-[(3S)-3-[4-[(1S)-2,2,2-trifluoro-1-[4-(4-morpholino-7H-pyrrolo[2,3-d]pyrimidin-6- |
| | yl)anilino]ethyl]-1-piperidyl]pyrrolidin-1-yl]prop-2-en-1-one |
| 959 | 1-[(3R)-3-[4-[(1S)-2,2,2-trifluoro-1-[4-(4-morpholino-7H-pyrrolo[2,3-d]pyrimidin-6- |
| | yl)anilino]ethyl]-1-piperidyl]pyrrolidin-1-yl]prop-2-en-1-one |
| 960 | 1-[4-[[5-[4-(4-morpholino-7H-pyrrolo[2,3-d]pyrimidin-6-yl)anilino]pyrimidin-2- |
| | yl]methyl]piperazin-1-yl]prop-2-en-1-one |
| 961 | N-[(3R)-1-[[5-[4-(4-morpholino-7H-pyrrolo[2,3-d]pyrimidin-6-yl)anilino]pyrimidin-2- |
| | yl\|methyl]-3-piperidyl]prop-2-enamide |
| 962 | 1-[(3S)-3-[(1S)-2,2,2-trifluoro-1-[4-(4-morpholino-7H-pyrrolo[2,3-d]pyrimidin-6- |
| | yl)anilino]ethyl]pyrrolidin-1-yl]prop-2-en-1-one |
| 963 | 1-[(3R)-3-[(3S)-3-[2,2,2-trifluoro-1-[4-(4-morpholino-7H-pyrrolo[2,3-d]pyrimidin-6- |
| | yl)anilino]ethyl]pyrrolidin-1-yl]pyrrolidin-1-yl]prop-2-en-1-one |
| 964 | 1-[(3S)-3-[4-[(1R)-2,2,2-trifluoro-1-[4-(4-morpholino-7H-pyrrolo[2,3-d]pyrimidin-6- |
| | yl)anilino]ethyl]-1-piperidyl]pyrrolidin-1-yl]prop-2-en-1-one |
| 965 | 1-[(3R)-3-[4-[(1R)-2,2,2-trifluoro-1-[4-(4-morpholino-7H-pyrrolo[2,3-d]pyrimidin-6- |
| | yl)anilino]ethyl]-1-piperidyl]pyrrolidin-1-yl]prop-2-en-1-one |
| 966 | N-[(3R)-3-methyl-1-[[5-[4-(4-morpholino-7H-pyrrolo[2,3-d]pyrimidin-6-yl)anilino]pyrimidin-2- |
| | yl]methyl]-3-piperidyl]prop-2-enamide |
| 967 | 1-[4-[(3R)-3-[(1R)-2,2,2-trifluoro-1-[4-(4-morpholino-7H-pyrrolo[2,3-d]pyrimidin-6- |
| | yl)anilino]ethyl]pyrrolidin-1-yl]-1-piperidyl]prop-2-en-1-one |
| 968 | (E)-4-(azetidin-1-yl)-1-[4-[5-[4-(4-morpholino-1H-imidazo[4,5-c]pyridin-2- |
| | yl)anilino]pyrimidin-2-yl]piperazin-1-yl]but-2-en-1-one;2,2,2-trifluoroacetic acid |
| 969 | 1-[4-[5-[4-[4-(4,4-difluoro-1-piperidyl)-7H-pyrrolo[2,3-d]pyrimidin-6-yl]anilino]pyrimidin-2- |
| | yl]piperazin-1-yl]prop-2-en-1-one |
| 970 | 1-[4-[5-[4-[4-(1,1-dioxo-1,4-thiazinan-4-yl)-7H-pyrrolo[2,3-d]pyrimidin-6-yl]anilino]pyrimidin- |
| | 2-yl┘piperazin-1-yl┘prop-2-en-1-one |
| 971 | 4-methoxy-1-[4-[5-[4-(4-morpholino-7H-pyrrolo[2,3-d]pyrimidin-6-yl)anilino]pyrimidin-2- |
| | yl]piperazin-1-yl]but-2-yn-1-one |
| 972 | 4-hydroxy-1-[4-[(1S)-2,2,2-trifluoro-1-[4-(4-morpholino-1H-pyrrolo[3,2-c]pyridin-2- |
| | yl)anilino]ethyl]-1-piperidyl]but-2-yn-1-one |
| 973 | N-[4-(4-morpholino-7H-pyrrolo[2,3-d]pyrimidin-6-yl)phenyl]-2-[3-[(E)-prop-1-enyl]sulfonyl-1- |
| | piperidyl]pyrimidin-5-amine |
| 1001 | (E)-4-(azetidin-1-yl)-1-[4-[3-[2,2,2-trifluoro-1-[4-(4-morpholino-7H-pyrrolo[2,3-d]pyrimidin-6- |
| | yl)anilino]ethyl]azetidin-1-yl]-1-piperidyl]but-2-en-1-one |
| 1002 | 4-(dimethylamino)-N-methyl-N-[(3R)-1-[5-[4-(4-morpholino-7H-pyrrolo[2,3-d]pyrimidin-6- |
| | yl)anilino]pyrimidin-2-yl]-3-piperidyl]but-2-ynamide |
| 1003 | 1-[4-[1-[4-[4-(4,4-difluoro-1-piperidyl)-7H-pyrrolo[2,3-d]pyrimidin-6-yl]anilino]-2,2,2- |
| | trifluoro-ethyl]-1-piperidyl]prop-2-en-1-one |
| 1004 | 1-[4-[1-[4-[4-(1,1-dioxo-1,4-thiazinan-4-yl)-7H-pyrrolo[2,3-d]pyrimidin-6-yl]anilino]-2,2,2- |
| | trifluoro-ethyl]-1-piperidyl]prop-2-en-1-one |
| 1005 | 1-[4-[5-[4-[4-(4,4-difluoro-1-piperidyl)-1H-pyrrolo[3,2-c]pyridin-2-yl]anilino]pyrimidin-2- |
| | yl]piperazin-1-yl]prop-2-en-1-one |
| 1006 | 1-[4-[(3R)-3-[(1R)-2,2,2-trifluoro-1-[4-(4-morpholino-7H-pyrrolo[2,3-d]pyrimidin-6- |
| | yl)anilino]ethyl]pyrrolidin-1-yl]-1-piperidyl]prop-2-en-1-one |
| 1007 | (E)-4-(dimethylamino)-1-[4-[5-[4-[4-(3-oxa-8-azabicyclo[3.2.1]octan-8-yl)-1H-imidazo[4,5- |
| | c]pyridin-2-yl]anilino]pyrimidin-2-yl]piperazin-1-yl]but-2-en-1-one |
| 1008 | 2-(azetidin-1-ylmethyl)-N-methyl-N-[(3R)-1-[5-[4-(4-morpholino-7H-pyrrolo[2,3-d]pyrimidin- |
| | 6-yl)anilino]pyrimidin-2-yl]-3-piperidyl]prop-2-enamide |
| 1009 | 2-(azetidin-1-ylmethyl)-N-methyl-N-[(3R)-1-[5-[4-(4-morpholino-7H-pyrrolo[2,3-d]pyrimidin- |
| | 6-yl)anilino]-2-pyridyl]-3-piperidyl]prop-2-enamide |
| 1010 | 1-[4-[(3S)-3-[(1R)-2,2,2-trifluoro-1-[4-(4-morpholino-7H-pyrrolo[2,3-d]pyrimidin-6- |
| | yl)anilino]ethyl]pyrrolidin-1-yl]-1-piperidyl]prop-2-en-1-one |
| 1011 | N-[4-(5-cyano-4-morpholino-1H-pyrrolo[2,3-b]pyridin-2-yl)phenyl]-4-[[(3R)-3-(prop-2- |
| | enoylamino)-1-piperidyl]methyl]pyridine-2-carboxamide |
| 1012 | 4-hydroxy-1-[4-[5-[4-(4-morpholino-1H-pyrrolo[3,2-c]pyridin-2-yl)anilino]pyrimidin-2- |
| | yl]piperazin-1-yl]but-2-yn-1-one |
| 1013 | N-[4-(4-morpholinoquinazolin-7-yl)phenyl]-4-[[(3R)-3-(prop-2-enoylamino)-1- |
| | piperidyl]methyl]pyridine-2-carboxamide |
| 1014 | 2-(azetidin-1-ylmethyl)-1-[4-[[5-[4-(4-morpholino-7H-pyrrolo[2,3-d]pyrimidin-6- |
| | yl)anilino┘pyrimidin-2-yl┘methyl┘piperazin-1-yl┘prop-2-en-1-one |
| 1015 | 1-[7-[2,2,2-trifluoro-1-[4-(4-morpholino-7H-pyrrolo[2,3-d]pyrimidin-6-yl)anilino]ethyl]-2- |
| | azaspiro[3.5]nonan-2-yl]prop-2-en-1-one |
| 1016 | N-[4-(4-morpholino-1H-pyrrolo[2,3-b]pyridin-2-yl)phenyl]-4-[[(3R)-3-(prop-2-enoylamino)-1- |
| | piperidyl]methyl]pyridine-2-carboxamide |
| 1017 | 1-[3-[4-[(1S)-2,2,2-trifluoro-1-[4-[4-(3-oxa-8-azabicyclo[3.2.1]octan-8-yl)-7H-pyrrolo[2,3- |
| | d]pyrimidin-6-yl]anilino]ethyl]-1-piperidyl]pyrrolidin-1-yl]but-2-yn-1-one |
| 1018 | 1-[(3S)-3-[(3S)-3-[2,2,2-trifluoro-1-[4-(4-morpholino-7H-pyrrolo[2,3-d]pyrimidin-6- |
| | yl)anilino]ethyl]pyrrolidin-1-yl]pyrrolidin-1-yl]prop-2-en-1-one |
| 1019 | 2-(azetidin-1-ylmethyl)-N-[(3R)-1-[5-[4-(4-morpholino-7H-pyrrolo[2,3-d]pyrimidin-6- |
| | yl)anilino]-2-pyridyl]-3-piperidyl]prop-2-enamide |
| 1020 | 1-[3-[4-[(1S)-2,2,2-trifluoro-1-[4-[4-(3-oxa-8-azabicyclo[3.2.1]octan-8-yl)-7H-pyrrolo[2,3- |
| | d]pyrimidin-6-yl]anilino]ethyl]-1-piperidyl]pyrrolidin-1-yl]prop-2-en-1-one |
| 1021 | 4-[[(3R)-3-(prop-2-enoylamino)-1-piperidyl]methyl]-N-[4-[4-(1H-pyrazol-3-yl)-7H-pyrrolo[2,3- |
| | d]pyrimidin-6-yl]phenyl]pyridine-2-carboxamide |
| 1022 | 1-[4-[5-[4-[4-(1,1-dioxo-1,4-thiazinan-4-yl)-1H-pyrrolo[3,2-c]pyridin-2-yl]anilino]pyrimidin-2- |
| | yl]piperazin-1-yl]prop-2-en-1-one |
| 1023 | 1-[4-[5-[4-(2-methyl-4-morpholino-7H-pyrrolo[2,3-d]pyrimidin-6-yl)anilino]pyrimidin-2- |
| | yl]piperazin-1-yl]prop-2-en-1-one |
| 1024 | 1-[4-[5-[4-(2-cyclopropyl-4-morpholino-7H-pyrrolo[2,3-d]pyrimidin-6-yl)anilino]pyrimidin-2- |
| | yl]piperazin-1-yl]prop-2-en-1-one |
| 1026 | 1-[3-[4-[(1S)-2,2,2-trifluoro-1-[4-[4-[(1S,4S)-2-oxa-5-azabicyclo[2.2.1]heptan-5-yl]-7H- |
| | pyrrolo[2,3-d]pyrimidin-6-yl]anilino]ethyl]-1-piperidyl]pyrrolidin-1-yl]but-2-yn-1-one |
| 1027 | 1-[3-[4-[(1S)-2,2,2-trifluoro-1-[4-[4-[(1S,4S)-2-oxa-5-azabicyclo[2.2.1]heptan-5-yl]-7H- |
| | pyrrolo[2,3-d]pyrimidin-6-yl]anilino]ethyl]-1-piperidyl]pyrrolidin-1-yl]prop-2-en-1-one |
| 1028 | 1-[3-[4-[(1S)-2,2,2-trifluoro-1-[4-(4-morpholino-1H-pyrrolo[3,2-c]pyridin-2-yl)anilino]ethyl]-1- |
| | piperidyl]pyrrolidin-1-yl]prop-2-en-1-one |
| 1029 | 1-[3-[4-[(1S)-2,2,2-trifluoro-1-[4-(4-morpholino-1H-pyrrolo[3,2-c]pyridin-2-yl)anilino]ethyl]-1- |
| | piperidyl]pyrrolidin-1-yl]but-2-yn-1-one |
| 1031 | 1-[4-[5-[4-(4-morpholino-7H-pyrrolo[2,3-d]pyrimidin-6-yl)benzoyl]pyrimidin-2-yl]piperazin-1- |
| | yl]prop-2-en-1-one |
| 1032 | N-[(3R)-1-[[2-[5-(4-morpholino-7H-pyrrolo[2,3-d]pyrimidin-6-yl)-1H-benzimidazol-2-yl]-4- |
| | pyridyl]methyl]-3-piperidyl]prop-2-enamide |
| 1033 | 1-[4-[5-[hydroxy-[4-(4-morpholino-7H-pyrrolo[2,3-d]pyrimidin-6-yl)phenyl]methyl]pyrimidin- |
| | 2-yl\|piperazin-1-yl\|prop-2-en-1-one |
| 1034 | 1-[4-[5-[4-(2-amino-4-morpholino-7H-pyrrolo[2,3-d]pyrimidin-6-yl)anilino]pyrimidin-2- |
| | yl]piperazin-1-yl]prop-2-en-1-one |
| 1035 | 1-[4-[5-[4-(2-methoxy-4-morpholino-7H-pyrrolo[2,3-d]pyrimidin-6-yl)anilino]pyrimidin-2- |
| | yl]piperazin-1-yl]prop-2-en-1-one |
| 1036 | 4-methoxy-1-[4-[5-[4-[4-(3-oxa-8-azabicyclo[3.2.1]octan-8-yl)-7H-pyrrolo[2,3-d]pyrimidin-6- |
| | yl]anilino]pyrimidin-2-yl]piperazin-1-yl]but-2-yn-1-one |
| 1037 | 4-[[(3R)-3-(4-hydroxybut-2-ynoylamino)-1-piperidyl]methyl]-N-[4-(4-morpholino-7H- |
| | pyrrolo[2,3-d]pyrimidin-6-yl)phenyl]pyridine-2-carboxamide |
| 1038 | 4-hydroxy-1-[4-[5-[4-[4-(3-oxa-8-azabicyclo[3.2.1]octan-8-yl)-7H-pyrrolo[2,3-d]pyrimidin-6- |
| | yl]anilino]pyrimidin-2-yl]piperazin-1-yl]but-2-yn-1-one |
| 1039 | 4-methoxy-1-[4-[(1R)-2,2,2-trifluoro-1-[4-[4-(3-oxa-8-azabicyclo[3.2.1]octan-8-yl)-7H- |
| | pyrrolo[2,3-d]pyrimidin-6-yl]anilino]ethyl]-1-piperidyl]but-2-yn-1-one |
| 1040 | 1-[4-[5-[[4-(4-morpholino-7H-pyrrolo[2,3-d]pyrimidin-6-yl)phenyl]methyl]pyrimidin-2- |
| | yl]piperazin-1-yl]prop-2-en-1-one |
| 1041 | 1-[4-[2,2,2-trifluoro-1-[4-(2-methyl-4-morpholino-7H-pyrrolo[2,3-d]pyrimidin-6- |
| | yl)anilino]ethyl]-1-piperidyl]prop-2-en-1-one |
| 1042 | N-[4-(4-morpholinopyrido[2,3-d]pyrimidin-7-yl)phenyl]-4-[[(3R)-3-(prop-2-enoylamino)-1- |
| | piperidyl]methyl]pyridine-2-carboxamide |
| 1044 | 1-[3-[4-[(1R)-1-[4-[4-(1,1-dioxo-1,4-thiazinan-4-yl)-1H-pyrrolo[3,2-c]pyridin-2-yl]anilino]- |
| | 2,2,2-trifluoro-ethyl]-1-piperidyl]pyrrolidin-1-yl]-4-methoxy-but-2-yn-1-one |
| 1045 | 1-[4-[(1S)-1-[4-[4-(1,1-dioxo-1,4-thiazinan-4-yl)-1H-pyrrolo[3,2-c]pyridin-2-yl]anilino]-2,2,2- |
| | trifluoro-ethyl]-1-piperidyl]-4-hydroxy-but-2-yn-1-one |
| 1046 | 4-hydroxy-1-[4-[(1S)-2,2,2-trifluoro-1-[4-(4-morpholino-7H-pyrrolo[2,3-d]pyrimidin-6- |
| | yl)anilino]ethyl]-1-piperidyl]pent-2-yn-1-one |
| 1047 | 4-hydroxy-1-[4-[(1S)-2,2,2-trifluoro-1-[4-(4-morpholino-7H-pyrrolo[2,3-d]pyrimidin-6- |
| | yl)anilino]ethyl]-1-piperidyl]but-2-yn-1-one |
| 1048 | 4-oxo-4-[4-oxo-4-[4-[(1S)-2,2,2-trifluoro-1-[4-(4-morpholino-7H-pyrrolo[2,3-d]pyrimidin-6- |
| | yl)anilino]ethyl]-1-piperidyl]but-2-ynoxy]butanoic acid |
| 1049 | 4-hydroxy-4-methyl-1-[4-[(1S)-2,2,2-trifluoro-1-[4-(4-morpholino-7H-pyrrolo[2,3-d]pyrimidin- |
| | 6-yl)anilino]ethyl]-1-piperidyl]pent-2-yn-1-one |
| 1050 | 2-(azetidin-1-ylmethyl)-N-[(3R)-1-[5-[4-[4-(3-oxa-8-azabicyclo[3.2.1]octan-8-yl)-7H- |
| | pyrrolo[2,3-d]pyrimidin-6-yl]anilino]pyrimidin-2-yl]-3-piperidyl]prop-2-enamide |
| 1051 | 4-hydroxy-N-[(3R)-1-[5-[4-(4-morpholino-7H-pyrrolo[2,3-d]pyrimidin-6-yl)anilino]pyrimidin- |
| | 2-yl┘-3-piperidyl┘but-2-ynamide |
| 1052 | N-[4-(4-morpholino-7H-pyrrolo[2,3-d]pyrimidin-6-yl)phenyl]-4-[[rac-(3R)-3-[2-[(4- |
| | benzoylpiperazin-1-yl)methyl]prop-2-enoylamino]-1-piperidyl]methyl]pyridine-2-carboxamide |
| 1053 | N-[4-(4-morpholino-7H-pyrrolo[2,3-d]pyrimidin-6-yl)phenyl]-4-[[rac-(3R)-3-[2-[[4-(pyridine-3- |
| | carbonyl)piperazin-1-yl]methyl]prop-2-enoylamino]-1-piperidyl]methyl]pyridine-2-carboxamide |
| 1054 | N-[4-(4-morpholino-7H-pyrrolo[2,3-d]pyrimidin-6-yl)phenyl]-4-[[rac-(3R)-3-[2-[(4- |
| | acetylpiperazin-1-yl)methyl]prop-2-enoylamino]-1-piperidyl]methyl]pyridine-2-carboxamide |
| 1055 | 3-[4-[(1S)-2,2,2-trifluoro-1-[4-(4-morpholino-7H-pyrrolo[2,3-d]pyrimidin-6-yl)anilino]ethyl]-1- |
| | piperidyl]propanoic acid |
| 1056 | 1-[4-[5-[4-[2-(methylamino)-4-morpholino-7H-pyrrolo[2,3-d]pyrimidin-6-yl]anilino]pyrimidin- |
| | 2-yl]piperazin-1-yl]prop-2-en-1-one |
| 1057 | 1-[3-[4-[(1R)-2,2,2-trifluoro-1-[4-[4-(3-oxa-8-azabicyclo[3.2.1]octan-8-yl)-1H-pyrrolo[3,2- |
| | c]pyridin-2-yl]anilino]ethyl]-1-piperidyl]pyrrolidin-1-yl]prop-2-en-1-one |
| 1058 | N-[4-(4-morpholino-7H-pyrrolo[2,3-d]pyrimidin-6-yl)phenyl]-4-[[rac-(3R)-3-[2-[[4-[rac-(2S)-2- |
| | acetamido-3-methyl-butanoyl]piperazin-1-yl]methyl]prop-2-enoylamino]-1- |
| | piperidyl]methyl]pyridine-2-carboxamide |
| 1059 | N-[4-(4-morpholino-7H-pyrrolo[2,3-d]pyrimidin-6-yl)phenyl]-4-[[rac-(3R)-3-[2-[[4-[rac-(2S)-2- |
| | acetamidopropanoyl]piperazin-1-yl]methyl]prop-2-enoylamino]-1-piperidyl]methyl]pyridine-2- |
| | carboxamide |
| 1060 | N-[4-(4-morpholino-7H-pyrrolo[2,3-d]pyrimidin-6-yl)phenyl]-4-[[rac-(3R)-3-[2-[(1,1-dioxo-1,4- |
| | thiazinan-4-yl)methyl]prop-2-enoylamino]-1-piperidyl]methyl]pyridine-2-carboxamide |
| 1061 | 4-hydroxy-4-methyl-N-[(3R)-1-[5-[4-(4-morpholino-7H-pyrrolo[2,3-d]pyrimidin-6- |
| | yl)anilino]pyrimidin-2-yl]-3-piperidyl]pent-2-ynamide |
| 1062 | 1-┌4-┌5-┌4-(4-morpholino-7H-pyrrolo┌2,3-d┐pyrimidin-6-yl)phenyl┐sulfonylpyrimidin-2- |
| | yl]piperazin-1-yl]prop-2-en-1-one |
| 1064 | N-[4-(4-morpholino-7H-pyrrolo[2,3-d]pyrimidin-6-yl)phenyl]-4-[[rac-(3R)-3-[2-[[4-(2- |
| | acetamidoacetyl)piperazin-1-yl]methyl]prop-2-enoylamino]-1-piperidyl]methyl]pyridine-2- |
| | carboxamide |
| 1065 | N-[4-(4-morpholino-7H-pyrrolo[2,3-d]pyrimidin-6-yl)phenyl]-4-[[rac-(3R)-3-[2-[(4-methyl-3- |
| | oxo-piperazin-1-yl)methyl]prop-2-enoylamino]-1-piperidyl]methyl]pyridine-2-carboxamide |
| 1066 | N,N-diethyl-4-[4-[(1S)-2,2,2-trifluoro-1-[4-(4-morpholino-7H-pyrrolo[2,3-d]pyrimidin-6- |
| | yl)anilino]ethyl]-1-piperidyl]but-2-ynamide |
| 1067 | 1-[4-[5-[4-(4-morpholino-7H-pyrrolo[2,3-d]pyrimidin-6-yl)phenoxy]pyrimidin-2-yl]piperazin-1- |
| | yl]prop-2-en-1-one |
| 1068 | N-[4-(4-morpholino-7H-pyrrolo[2,3-d]pyrimidin-6-yl)phenyl]-4-[[rac-(3R)-3-[2-[(4- |
| | methylsulfonylpiperazin-1-yl)methyl]prop-2-enoylamino]-1-piperidyl]methyl]pyridine-2- |
| | carboxamide |
| 1069 | N-[4-(4-morpholino-7H-pyrrolo[2,3-d]pyrimidin-6-yl)phenyl]-4-[[rac-(3R)-3-[2-[[3- |
| | (trifluoromethyl)pyrrolidin-1-yl]methyl]prop-2-enoylamino]-1-piperidyl]methyl]pyridine-2- |
| | carboxamide |
| 1070 | N-[4-(4-morpholino-7H-pyrrolo[2,3-d]pyrimidin-6-yl)phenyl]-4-[[rac-(3R)-3-[2-[[4-[rac-(2R)-2- |
| | acetamido-3-methyl-butanoyl]piperazin-1-yl]methyl]prop-2-enoylamino]-1- |
| | piperidyl]methyl]pyridine-2-carboxamide |
| 1071 | N-[4-(4-morpholino-7H-pyrrolo[2,3-d]pyrimidin-6-yl)phenyl]-4-[[rac-(3R)-3-[2-[[4-[rac-(2R)-2- |
| | acetamidopropanoyl]piperazin-1-yl]methyl]prop-2-enoylamino]-1-piperidyl]methyl]pyridine-2- |
| | carboxamide |
| 1072 | 1-[4-[5-[(R)-hydroxy-[4-(4-morpholino-7H-pyrrolo[2,3-d]pyrimidin-6- |
| | yl)phenyl]methyl]pyrimidin-2-yl]piperazin-1-yl]prop-2-en-1-one |
| 1073 | 1-[4-[5-[methoxy-[4-(4-morpholino-7H-pyrrolo[2,3-d]pyrimidin-6-yl)phenyl]methyl]pyrimidin- |
| | 2-yl]piperazin-1-yl]prop-2-en-1-one |
| 1074 | 2-(azetidin-1-ylmethyl)-N-[(3R)-1-[5-[hydroxy-[4-(4-morpholino-7H-pyrrolo[2,3-d]pyrimidin-6- |
| | yl)phenyl]methyl]pyrimidin-2-yl]-3-piperidyl]prop-2-enamide |
| 1075 | N-[4-(4-morpholino-7H-pyrrolo[2,3-d]pyrimidin-6-yl)phenyl]-4-[[rac-(3R)-3-[2-[(4,4-difluoro- |
| | 1-piperidyl)methyl]prop-2-enoylamino]-1-piperidyl]methyl]pyridine-2-carboxamide |
| 1076 | N-[4-(4-morpholino-7H-pyrrolo[2,3-d]pyrimidin-6-yl)phenyl]-4-[[rac-(3R)-3-[2-(2-oxa-6- |
| | azaspiro[3.3]heptan-6-ylmethyl)prop-2-enoylamino]-1-piperidyl]methyl]pyridine-2-carboxamide |
| 1077 | N-[4-(4-morpholino-7H-pyrrolo[2,3-d]pyrimidin-6-yl)phenyl]-4-[[rac-(3R)-3-[2-[[7-(pyridine-3- |
| | carbonyl)-2,7-diazaspiro[3.4]octan-2-yl]methyl]prop-2-enoylamino]-1- |
| | piperidyl]methyl]pyridine-2-carboxamide |
| 1078 | N-[4-(4-morpholino-7H-pyrrolo[2,3-d]pyrimidin-6-yl)phenyl]-4-[[rac-(3R)-3-[2-[(7-acetyl-2,7- |
| | diazaspiro[3.4]octan-2-yl)methyl]prop-2-enoylamino]-1-piperidyl]methyl]pyridine-2- |
| | carboxamide |
| 1079 | N-[(3R)-1-[[1-[[4-(4-morpholino-7H-pyrrolo[2,3-d]pyrimidin-6-yl)phenyl]methyl]-2-oxo-4- |
| | pyridyl]methyl]-3-piperidyl]prop-2-enamide |
| 1080 | 1-┌4-┌5-┌hydroxy-┌4-(4-morpholino-1H-pyrrolo┌3,2-c┐pyridin-2-yl)phenyl┐methyl┐pyrimidin-2- |
| | yl]piperazin-1-yl]prop-2-en-1-one |
| 1081 | N,N-diethyl-4-[4-[(1R)-2,2,2-trifluoro-1-[4-(4-morpholino-7H-pyrrolo[2,3-d]pyrimidin-6- |
| | yl)anilino]ethyl]-1-piperidyl]but-2-ynamide |
| 1082 | N-[(3R)-1-[5-[4-(4-morpholino-7H-pyrrolo[2,3-d]pyrimidin-6-yl)anilino]pyrimidin-2-yl]-3- |
| | piperidyl]-2-(2-oxa-6-azaspiro[3.3]heptan-6-ylmethyl)prop-2-enamide |
| 1083 | 1-[3-[5-[4-(4-morpholino-7H-pyrrolo[2,3-d]pyrimidin-6-yl)anilino]pyrimidin-2-yl]-3,8- |
| | diazabicyclo[3.2.1]octan-8-yl]prop-2-en-1-one |
| 1084 | 2-(azetidin-1-ylmethyl)-1-[7-[2,2,2-trifluoro-1-[4-(4-morpholino-7H-pyrrolo[2,3-d]pyrimidin-6- |
| | yl)anilino]ethyl]-2-azaspiro[3.5]nonan-2-yl]prop-2-en-1-one |
| 1085 | 1-[4-[5-[(S)-hydroxy-[4-(4-morpholino-7H-pyrrolo[2,3-d]pyrimidin-6- |
| | yl)phenyl]methyl]pyrimidin-2-yl]piperazin-1-yl]prop-2-en-1-one |
| 1086 | N,N-diethyl-4-└4-└5-└hydroxy-└4-(4-morpholino-7H-pyrrolo└2,3-d┘pyrimidin-6- |
| | yl)pheny]methyl]pyrimidin-2-yl]piperazin-1-yl]but-2-ynamide |
| 1087 | 1-[8-[5-[4-(4-morpholino-7H-pyrrolo[2,3-d]pyrimidin-6-yl)anilino]pyrimidin-2-yl]-3,8- |
| | diazabicyclo[3.2.1]octan-3-yl]prop-2-en-1-one |
| 1088 | 2-[[4-(pyridine-3-carbonyl)piperazin-1-yl]methyl]-N-[rac-(3R)-1-[5-[4-(4-morpholino-7H- |
| | pyrrolo[2,3-d]pyrimidin-6-yl)anilino]pyrimidin-2-yl]-3-piperidyl]prop-2-enamide |
| 1089 | 2-[(4-acetylpiperazin-1-yl)methyl]-N-[rac-(3R)-1-[5-[4-(4-morpholino-7H-pyrrolo[2,3- |
| | d]pyrimidin-6-yl)anilino]pyrimidin-2-yl]-3-piperidyl]prop-2-enamide |
| 1091 | 2-(azetidin-1-ylmethyl)-1-[3-[5-[4-(4-morpholino-7H-pyrrolo[2,3-d]pyrimidin-6- |
| | yl)anilino]pyrimidin-2-yl]-3,8-diazabicyclo[3.2.1]octan-8-yl]prop-2-en-1-one |
| 1092 | 1-[8-[5-[hydroxy-[4-(4-morpholino-7H-pyrrolo[2,3-d]pyrimidin-6-yl)phenyl]methyl]pyrimidin- |
| | 2-yl]-3,8-diazabicyclo[3.2.1]octan-3-yl]prop-2-en-1-one |
| 1093 | 1-[3-[5-[hydroxy-[4-(4-morpholino-7H-pyrrolo[2,3-d]pyrimidin-6-yl)phenyl]methyl]pyrimidin- |
| | 2-yl]-3,8-diazabicyclo[3.2.1]octan-8-yl]prop-2-en-1-one |
| 1094 | N-[4-(4-morpholino-7H-pyrrolo[2,3-d]pyrimidin-6-yl)phenyl]-4-[[rac-(3R)-3-[2-[[rac-(2R,6S)- |
| | 2,6-dimethyl-1-piperidyl]methyl]prop-2-enoylamino]-1-piperidyl]methyl]pyridine-2- |
| | carboxamide |
| 1095 | N-[4-(4-morpholino-7H-pyrrolo[2,3-d]pyrimidin-6-yl)phenyl]-4-[[rac-(3R)-3-[2-(8- |
| | azabicyclo[3.2.1]octan-8-ylmethyl)prop-2-enoylamino]-1-piperidyl]methyl]pyridine-2- |
| | carboxamide |
| 1096 | N-[4-(4-morpholino-7H-pyrrolo[2,3-d]pyrimidin-6-yl)phenyl]-4-[[rac-(3R)-3-[[rac-(3R)-3- |
| | amino-2-methylene-butanoyl]amino]-1-piperidyl]methyl]pyridine-2-carboxamide |
| 1097 | N-[4-(4-morpholino-7H-pyrrolo[2,3-d]pyrimidin-6-yl)phenyl]-4-[[rac-(3R)-3-[[rac-(3R)-3- |
| | (dimethylamino)-2-methylene-butanoyl]amino]-1-piperidyl]methyl]pyridine-2-carboxamide |
| 1098 | 4-ethoxy-N-┌(3R)-1-┌5-┌4-(4-morpholino-7H-pyrrolo┌2,3-d┐pyrimidin-6-yl)anilino┐pyrimidin-2- |
| | yl]-3-piperidyl]but-2-ynamide |
| 1099 | (E)-4-(dimethylamino)-1-[8-[5-[hydroxy-[4-(4-morpholino-7H-pyrrolo[2,3-d]pyrimidin-6- |
| | yl)phenyl]methyl]pyrimidin-2-yl]-3,8-diazabicyclo[3.2.1]octan-3-yl]but-2-en-1-one |
| 1100 | 2-(azetidin-1-ylmethyl)-N-[(3R)-3-methyl-1-[5-[4-(4-morpholino-7H-pyrrolo[2,3-d]pyrimidin-6- |
| | yl)anilino]pyrimidin-2-yl]-3-piperidyl]prop-2-enamide |
| 1101 | 2-(azetidin-1-ylmethyl)-N-[(3R)-1-[5-[hydroxy-[4-(4-morpholino-7H-pyrrolo[2,3-d]pyrimidin-6- |
| | yl)phenyl]methyl]pyrimidin-2-yl]-3-methyl-3-piperidyl]prop-2-enamide |
| 1102 | (E)-4-(dimethylamino)-1-[8-[5-[4-(4-morpholino-7H-pyrrolo[2,3-d]pyrimidin-6- |
| | yl)anilino]pyrimidin-2-yl]-3,8-diazabicyclo[3.2.1]octan-3-yl]but-2-en-1-one |
| 1103 | 1-[4-[5-[2,2,2-trifluoro-1-hydroxy-1-[4-(4-morpholino-7H-pyrrolo[2,3-d]pyrimidin-6- |
| | yl)phenyl]ethyl]pyrimidin-2-yl]piperazin-1-yl]prop-2-en-1-one |

In certain embodiments, the compound is Compound #626, 705, 906, 915, 971, 1033, or 1036. In certain embodiments, the compound is Compound #915, 1033, 1036, 1087, 1092, 1094, 1095, 1097, 1098, or 1102.

Embodiments of the compounds of Formula (I) display improved potency against menin with IC₅₀ values of as low as less than 1 nM or less than 0.1 nM, and/or high occupancy of active site of menin (e.g., more than 50%, 70% or 90% occupancy) at low dosages of below 5 mg/kg (e.g., at or below 3 mg/kg) when administered in vivo (e.g., in rats).

In some embodiments, provided herein is a pharmaceutical composition comprising a compound according to formula (L-I), (L-II), or (I).

In some embodiments, provided herein is a pharmaceutical composition comprising a therapeutically effective amount of a compound of formula (L-I), (L-II), or (I), and a pharmaceutically acceptable excipient.

In some embodiments, the pharmaceutical composition is formulated for a route of administration selected from oral administration, parenteral administration, buccal administration, nasal administration, topical administration, or rectal administration.

In some embodiments, provided herein are methods for treating an autoimmune disease or condition comprising administering to a patient in need the pharmaceutical composition provided herein.

In some embodiments, the autoimmune disease is selected from rheumatoid arthritis or lupus.

In some embodiments, provided herein are methods for treating a heteroimmune disease or condition comprising administering to a patient in need the pharmaceutical composition provided herein.

In some embodiments, provided herein are methods for treating a cancer comprising administering to a patient in need the pharmaceutical composition provided herein.

In some embodiments, the cancer is a B-cell proliferative disorder.

In some embodiments, the B-cell proliferative disorder is diffuse large B cell lymphoma, follicular lymphoma, or chronic lymphocytic leukemia. In some embodiments, the disorder is myeloid leukemia. In some embodiments, the disorder is acute myeloid leukemia (AML). In some embodiments, the B-cell proliferative disorder is lymphoid leukemia. In some embodiments, the disorder is acute lymphocytic leukemia (ALL). In some embodiments, the disorder is soft tissue tumors. In some embodiments, the tumor is glioblastoma. In some embodiments, the tumor is pancreatic tumor. In some embodiments, the disorder is renal cell cancer.

In some embodiments, the disorder is KRas mutated solid tumors.

In some embodiments, the disorder is multiple myeloma.

In some embodiments, the disorder is Triple-Hit Lymphoma (THL).

In some embodiments, the disorder is Double Expresser Lymphoma (DEL). In some embodiments, the disorder is DLBCL.

In some embodiments, provided herein are methods for treating mastocytosis comprising administering to a patient in need the pharmaceutical composition provided herein.

In some embodiments, provided herein are methods for treating osteoporosis or bone resorption disorders comprising administering to a patient in need the pharmaceutical composition provided herein.

In some embodiments, provided herein are methods for treating an inflammatory disease or condition comprising administering to a patient in need the pharmaceutical composition provided herein.

In some embodiments, provided herein are methods for treating lupus comprising administering to a subject in need thereof a composition containing a therapeutically effective amount of a compound of formula (L-I), (L-II), or (I) that is inhibitor of menin or menin-MLL interaction.

In some embodiments, provided herein are methods for treating a heteroimmune disease or condition comprising administering to a subject in need thereof a composition containing a therapeutically effective amount of a compound of formula (L-I), (L-II), or (I) that is inhibitor of menin or menin-MLL interaction.

In some embodiments provided herein are methods for treating diffuse large B cell lymphoma, follicular lymphoma or chronic lymphocytic leukemia comprising administering to a subject in need thereof a composition containing a therapeutically effective amount of a compound of formula (L-I), (L-II), or (I) that is inhibitor of the menin or menin-MLL interaction.

In some embodiments, provided herein are methods for treating mastocytosis, comprising administering to a subject in need thereof a composition containing a therapeutically effective amount of a compound of formula (L-I), (L-II), or (I) that is inhibitor of menin or menin-MLL interaction.

In some embodiments, provided herein are methods for treating osteoporosis or bone resorption disorders comprising administering to a subject in need thereof a composition containing a therapeutically effective amount of a compound of formula (L-I), (L-II), or (I) that is inhibitor of menin or menin-MLL interaction.

In some embodiments, provided herein are methods for treating an inflammatory disease or condition comprising administering to a subject in need thereof a composition containing a therapeutically effective amount of a compound of formula (L-I), (L-II), or (I) that is inhibitor of menin-MLL interaction.

In some embodiments, provided herein is a pharmaceutical composition comprising a pharmaceutically acceptable carrier and a pharmaceutically effective amount of a compound according to any one of the formulas described herein. In some embodiments, the compound is according to any one of Formula (L-I), (L-II), or (I).

In some embodiments, the pharmaceutical composition is formulated for a route of administration selected from oral administration, parenteral administration, buccal administration, nasal administration, topical administration, or rectal administration.

In some embodiments, the carrier is a parenteral carrier.

In some embodiments, the carrier is an oral carrier.

In some embodiments, the carrier is a topical carrier.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

Further representative embodiments of compounds of Formula (L-I), include compounds listed in Table 1C-1F, or a pharmaceutically acceptable salt, solvate, hydrate, or stereoisomer thereof.

In some embodiments, provided herein are methods for preventing, treating or ameliorating in a mammal a disease or condition that is causally related to the aberrant activity of a menin-MLL interaction in vivo, which comprises administering to the mammal an effective disease-treating or condition-treating amount of any of the compounds listed in Table 1E.

Throughout the specification, groups and substituents thereof can be chosen by one skilled in the field to provide stable moieties and compounds.

In some embodiments, the compounds of Formula (L-I), (L-II), and (I) inhibit menin. In some embodiments, the compounds of Formula (L-I), (L-II), and (I) are used to treat patients suffering from menin mediated conditions or diseases, including, but not limited to, cancer, autoimmune and other inflammatory diseases.

In some embodiments, the compounds of Formula (L-I), (L-II), and (I) inhibit menin-MLL. In some embodiments, the compounds of Formula (L-I), (L-II), and (I) are used to treat patients suffering from menin, menin-MLL-dependent or menin-MLL interaction mediated conditions or diseases, including, but not limited to, cancer, autoimmune and other inflammatory diseases.

In some embodiments, the compounds of Formula (L-I), (L-II), and (I) inhibit menin-MLL interaction. In some embodiments, the compounds of Formula (L-I), (L-II), and (I) are used to treat patients suffering from menin activity or menin-MLL interaction-dependent or menin-MLL interaction mediated conditions or diseases, including, but not limited to, cancer, autoimmune and other inflammatory diseases.

### Preparation of Compounds

Compounds of any of formula described herein or Formula (L-I), (L-II), and (I) may be synthesized using standard synthetic reactions known to those of skill in the art or using methods known in the art. The reactions can be employed in a linear sequence to provide the compounds or they may be used to synthesize fragments which are subsequently joined by the methods known in the art. Exemplary methods are provided in the Examples herein.

Described herein are compounds that inhibit the activity of menin or menin-MLL, and processes for their preparation. Also described herein are pharmaceutically acceptable salts, pharmaceutically acceptable solvates, pharmaceutically active metabolites, and pharmaceutically acceptable prodrugs of such compounds. Pharmaceutical compositions that include at least one such compound or a pharmaceutically acceptable salt, pharmaceutically acceptable solvate, pharmaceutically active metabolite or pharmaceutically acceptable prodrug of such compound, are provided.

The starting material used for the synthesis of the compounds described herein may be synthesized or can be obtained from commercial sources, such as, but not limited to, Aldrich Chemical Co. (Milwaukee, Wisconsin), Bachem (Torrance, California), or Sigma Chemical Co. (St. Louis, Mo.). The compounds described herein, and other related compounds having different substituents can be synthesized using techniques and materials known to those of skill in the art, such as described, for example, in March, ADVANCED ORGANIC CHEMISTRY 4th Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989). (all of which are incorporated by reference in their entirety). Additional methods for the synthesis of compounds described herein may be found in International Patent Publication No. WO 01/01982901, Arnold et al. Bioorganic & Medicinal Chemistry Letters 10 (2000) 2167-2170; Burchat et al. Bioorganic & Medicinal Chemistry Letters 12 (2002) 1687-1690. General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein.

The products of the reactions may be isolated and purified, if desired, using conventional techniques, including, but not limited to, filtration, distillation, crystallization, chromatography, and the like. Such materials may be characterized using conventional means, including physical constants and spectral data.

Compounds described herein may be prepared as a single isomer or a mixture of isomers.

In some embodiments, representative compounds of Formula (I) are prepared according to synthetic schemes depicted herein.

### Further Forms of Compounds

Compounds disclosed herein have a structure of Formula (L-I), (L-II), and (I). It is understood that when reference is made to compounds described herein, it is meant to include compounds of any of formula described herein or Formula (L-I), (L-II), and (I) as well as to all of the specific compounds that fall within the scope of these generic formulae, unless otherwise indicated.

Compounds described herein may possess one or more stereocenters and each center may exist in the R or S configuration. Compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

Diastereomeric mixtures can be separated into their individual diastereomers on the basis of their physical chemical differences by methods known, for example, by chromatography and/or fractional crystallization. In some embodiments, enantiomers can be separated by chiral chromatographic columns. In some embodiments, enantiomers can be separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

Methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, compounds described herein can exist in unsolvated as well as solvated forms with pharmaceutically acceptable solvents such as water, ethanol, and the like. Solvated forms of compounds presented herein are also considered to be disclosed herein.

Compounds of any of formula described herein or Formula (L-I), (L-II), and (I) in unoxidized form can be prepared from N-oxides of compounds of any of formula described herein or Formula (L-I), (L-II), and (I) by treating with a reducing agent, such as, but not limited to, sulfur, sulfur dioxide, triphenyl phosphine, lithium borohydride, sodium borohydride, phosphorus trichloride, tribromide, or the like in a suitable inert organic solvent, such as, but not limited to, acetonitrile, ethanol, aqueous dioxane, or the like at 0 to 80° C.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically, or therapeutically active form of the compound. In certain embodiments, a prodrug is enzymatically metabolized by one or more steps or processes to the biologically, pharmaceutically, or therapeutically active form of the compound. To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985).

Prodrug forms of the herein described compounds, wherein the prodrug is metabolized in vivo to produce a derivative as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. Prodrugs may be designed as reversible drug derivatives, for use as modifiers to enhance drug transport to site-specific tissues. In some embodiments, the design of a prodrug increases the effective water solubility. See, e.g., Fedorak et al., Am. J. Physiol., 269: G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Sites on the aromatic ring portion of compounds of any of formula described herein or Formula (L-I), (L-II), and (I) can be susceptible to various metabolic reactions, therefore incorporation of appropriate substituents on the aromatic ring structures, such as, by way of example only, halogens can reduce, minimize or eliminate this metabolic pathway.

Compounds described herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

In additional or some embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like.

The corresponding counterions of the pharmaceutically acceptable salts may be analyzed and identified using various methods including, but not limited to, ion exchange chromatography, ion chromatography, capillary electrophoresis, inductively coupled plasma, atomic absorption spectroscopy, mass spectrometry, or any combination thereof.

The salts are recovered by using at least one of the following techniques: filtration, precipitation with a non-solvent followed by filtration, evaporation of the solvent, or, in the case of aqueous solutions, lyophilization.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and they may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

It should be understood that a reference to a salt includes the solvent addition forms or crystal forms thereof, particularly solvates or polymorphs. Solvates contain either stoichiometric or non-stoichiometric amounts of a solvent, and are often formed during the process of crystallization with pharmaceutically acceptable solvents such as water, ethanol, and the like. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

Compounds described herein may be in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The screening and characterization of the pharmaceutically acceptable salts, polymorphs, and/or solvates may be accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UVIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

Throughout the specification, groups and substituents thereof can be chosen by one skilled in the field to provide stable moieties and compounds.

### Pharmaceutical Composition/Formulation

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

A pharmaceutical composition, as used herein, refers to a mixture of a compound described herein, such as, for example, compounds of any of formula described herein or Formula (L-I), (L-II), and (I) with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, compositions may also include one or more salts in an amount required to bring osmolality of the composition into an acceptable range. Such salts include those having sodium, potassium or ammonium cations, and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The pharmaceutical compositions described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical compositions described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound described herein, such as, for example, a compound of any of formula described herein or Formula (L-I), (L-II), and (I) as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite, and tocopherol. In certain embodiments, antioxidants enhance chemical stability where required.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v, polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of any of formula described herein or Formula (L-I), (L-II), and (I) and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizcers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per ml, dl, or 1 of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. As used herein, the term “subject” is used to mean an animal, preferably a mammal, including a human or non-human. The terms patient and subject may be used interchangeably.

Moreover, the pharmaceutical compositions described herein, which include a compound of any of formula described herein or Formula (L-I), (L-II), and (I) can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose: or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In some embodiments, the pharmaceutical composition is in the form of a powder. In some embodiments, the pharmaceutical composition is in the form of a tablet, including but not limited to, a fast-melt tablet. Additionally, pharmaceutical compositions described herein may be administered as a single capsule or in multiple capsule dosage form. In some embodiments, the pharmaceutical composition is administered in two, or three, or four, capsules or tablets.

In some embodiments, solid dosage forms, e.g., tablets, effervescent tablets, and capsules, are prepared by mixing particles of a compound of any of formula described herein or Formula (L-I), (L-II), and (I) with one or more pharmaceutical excipients to form a bulk blend composition. When referring to these bulk blend compositions as homogeneous, it is meant that the particles of the compound of any of formula described herein or Formula (L-I), (L-II), and (I) are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some embodiments, using standard coating procedures, such as those described in Remington's Pharmaceutical Sciences, 20th Edition (2000), a film coating is provided around the formulation of the compound of any of Formula (L-I), (L-II), and (I). In some embodiments, some or all of the particles of the compound of any of formula described herein or Formula (L-I), (L-II), and (I) are coated. In some embodiments, some or all of the particles of the compound of any of Formula (L-I), (L-II), and (I), are microencapsulated. In still some embodiments, the particles of the compound of any of formula described herein or Formula (L-I), (L-II), and (I) are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound of any of formula described herein or Formula (L-I), (L-II), and (I) from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In some embodiments, one or more layers of the pharmaceutical composition are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In some embodiments, the compressed tablets will include a film surrounding the final compressed tablet. In some embodiments, the film coating can provide a delayed release of the compound of any of formula described herein or Formula (L-I), (L-II), and (I) from the formulation. In some embodiments, the film coating aids in patient compliance (e.g., Opadry® coatings or sugar coating). Film coatings including Opadry® typically range from about 1% to about 3% of the tablet weight. In some embodiments, the compressed tablets include one or more excipients.

A capsule may be prepared, for example, by placing the bulk blend of the formulation of the compound of any of Formula (L-I), (L-II), and (I), described above, inside of a capsule. In some embodiments, the formulations (non-aqueous suspensions and solutions) are placed in a soft gelatin capsule. In some embodiments, the formulations are placed in standard gelatin capsules or non-gelatin capsules such as capsules comprising HPMC. In some embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of the compound of any of formula described herein or Formula (L-I), (L-II), and (I) and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In some embodiments, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with compounds of any of formula described herein or Formula (L-I), (L-II), and (I) which sufficiently isolate the compound of any of formula described herein or Formula (L-I), (L-II), and (I) from other non-compatible excipients. Materials compatible with compounds of any of formula described herein or Formula (L-I), (L-II), and (I) are those that delay the release of the compounds of any of Formula (L-I), (L-II), and (I), in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In some embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In some embodiments, the microencapsulating material useful for delaying the release of the pharmaceutical compositions is from the USP or the National Formulary (NF). In some embodiments, the microencapsulation material is Klucel. In some embodiments, the microencapsulation material is methocel.

Microencapsulated compounds of any of formula described herein or Formula (L-I), (L-II), and (I) may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In some embodiments, the particles of compounds of any of formula described herein or Formula (L-I), (L-II), and (I) are microencapsulated prior to being formulated into one of the above forms. In still some embodiments, some or most of the particles are coated prior to being further formulated by using standard coating procedures, such as those described in Remington's Pharmaceutical Sciences, 20th Edition (2000).

In some embodiments, the solid dosage formulations of the compounds of any of formula described herein or Formula (L-I), (L-II), and (I) are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In some embodiments, a powder including the formulations with a compound of any of Formula (I)—(XIc), described herein, may be formulated to include one or more pharmaceutical excipients and flavors. Such a powder may be prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still some embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid, and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the formulations described herein, which include a compound of Formula (I), are solid dispersions. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl 2004/0013734, each of which is specifically incorporated by reference. In some embodiments, the formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions are known in the art and include, but are not limited to, for example, U.S. Pat. Nos. 4,151,273, 5,281,420, and 6,083,518, each of which is specifically incorporated by reference.

The pharmaceutical solid oral dosage forms including formulations described herein, which include a compound of any of formula described herein or Formula (L-I), (L-II), and (I) can be further formulated to provide a controlled release of the compound of Formula (I). Controlled release refers to the release of the compound of any of formula described herein or Formula (L-I), (L-II), and (I) from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In some embodiments, the polymers and compatible mixtures thereof, and some of their properties, include, but are not limited to:
Shellac, also called purified lac, a refined product obtained from the resinous secretion of an insect. This coating dissolves in media of pH>7;
Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS, and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;
Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;
Poly Vinyl Acetate Phthalate (PVAP). PVAP dissolves in pH>5, and it is much less permeable to water vapor gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate, and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In some embodiments, the formulations described herein, which include a compound of Formula (I), are delivered using a pulsatile dosage form. A pulsatile dosage form is capable of providing one or more immediate release pulses at predetermined time points after a controlled lag time or at specific sites. Pulsatile dosage forms including the formulations described herein, which include a compound of any of formula described herein or Formula (L-I), (L-II), and (I) may be administered using a variety of pulsatile formulations known in the art. For example, such formulations include, but are not limited to, those described in U.S. Pat. Nos. 5,011,692, 5,017,381, 5,229,135, and 5,840,329, each of which is specifically incorporated by reference. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284, all of which are specifically incorporated by reference. In some embodiments, the controlled release dosage form is pulsatile release solid oral dosage form including at least two groups of particles, (i.e. multiparticulate) each containing the formulation described herein. The first group of particles provides a substantially immediate dose of the compound of any of formula described herein or Formula (L-I), (L-II), and (I) upon ingestion by a mammal. The first group of particles can be either uncoated or include a coating and/or sealant. The second group of particles includes coated particles, which includes from about 2% to about 75%, from about 2.5% to about 70%, or from about 40% to about 70%, by weight of the total dose of the compound of any of formula described herein or Formula (L-I), (L-II), and (I) in said formulation, in admixture with one or more binders. The coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the formulation that includes a compound of any of Formula (I).

Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014, and 6,932,983, each of which is specifically incorporated by reference.

In some embodiments, pharmaceutical compositions are provided that include particles of the compounds of any of Formula (L-I), (L-II), and (I), described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition to the particles of compound of Formula (I), the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In some embodiments, an aqueous suspension can be re-suspended into a homogenous suspension by physical agitation lasting less than 1 minute. In some embodiments, an aqueous suspension can be re-suspended into a homogenous suspension by physical agitation lasting less than 45 seconds. In yet some embodiments, an aqueous suspension can be re-suspended into a homogenous suspension by physical agitation lasting less than 30 seconds. In still some embodiments, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions® described herein are known in the art and include, for example, hydrophilic polymers, electrolytes, Tween 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In some embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In some embodiments, the aqueous liquid dispersion can comprise a sweetening agent or flavoring agent in a concentration ranging from about 0.001% to about 1.0% the volume of the aqueous dispersion. In some embodiments, the aqueous liquid dispersion can comprise a sweetening agent or flavoring agent in a concentration ranging from about 0.005% to about 0.5% the volume of the aqueous dispersion. In yet some embodiments, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical compositions described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Intranasal Formulations

Intranasal formulations are known in the art and are described in, for example, U.S. Pat. Nos. 4,476,116, 5,116,817, and 6,391,452, each of which is specifically incorporated by reference. Formulations that include a compound of any of formula described herein or Formula (L-I), (L-II), and (I) which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. The nasal dosage form should be isotonic with nasal secretions.

For administration by inhalation, the compounds of any of Formula (L-I), (L-II), and (I), described herein may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

### Buccal Formulations

Buccal formulations that include compounds of any of formula described herein or Formula (L-I), (L-II), and (I) may be administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery of the compound of any of Formula (L-I), (L-II), and (I), is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with the compound of any of Formula (L-I), (L-II), and (I), and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

### Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

The transdermal dosage forms described herein may incorporate certain pharmaceutically acceptable excipients which are conventional in the art. In some embodiments, the transdermal formulations described herein include at least three components: (1) a formulation of a compound of any of Formula (I); (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In some embodiments, the transdermal formulations described herein can maintain a saturated or supersaturated state to promote diffusion into the skin.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of the compounds of any of Formula (L-I), (L-II), and (I). The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

Formulations that include a compound of any of Formula (L-I), (L-II), and (I), suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions, or emulsions in oily or aqueous vehicles, and may contain formulatory agents such as suspending, stabilizing and/or dispersing agents. Pharmaceutical compositions for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

### Formulations

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams, or ointments. Such pharmaceutical compounds can contain solubilizers, stabilizers, tonicity enhancing agents, buffers, and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

The compounds described herein can be used in the preparation of medicaments for the inhibition of menin or a homolog thereof, or for the treatment of diseases or conditions that would benefit, at least in part, from inhibition of menin or a homolog thereof. In addition, a method for treating any of the diseases or conditions described herein in a subject in need of such treatment, involves administration of pharmaceutical compositions containing at least one compound of any of Formula (L-I), (L-II), and (I), described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%- 100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Combination Treatments

The menin or menin-MLL inhibitor compositions described herein can also be used in combination with other well known therapeutic reagents that are selected for their therapeutic value for the condition to be treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In certain instances, it may be appropriate to administer at least one menin or menin-MLL inhibitor compound described herein in combination with another therapeutic agent. By way of example only, if one of the side effects experienced by a patient upon receiving one of the menin or menin-MLL inhibitor compounds described herein is nausea, then it may be appropriate to administer an anti-nausea agent in combination with the initial therapeutic agent. Or, by way of example only, the therapeutic effectiveness of one of the compounds described herein may be enhanced by administration of an adjuvant (i.e., by itself the adjuvant may have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature Combination treatment further includes periodic treatments that start and stop at various times to assist with the clinical management of the patient.

For combination therapies described herein, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

In any case, the multiple therapeutic agents (one of which is a compound of Formula (L-I), (L-II), and (I), described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; the use of multiple therapeutic combinations are also envisioned.

It is understood that the dosage regimen to treat, prevent, or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life, and kinetic profile of the pharmaceutical agent. Circadian variation of the target molecule concentration may also determine the optimal dose interval.

In addition, the compounds described herein also may be used in combination with procedures that may provide additional or synergistic benefit to the patient. By way of example only, patients are expected to find therapeutic and/or prophylactic benefit in the methods described herein, wherein pharmaceutical composition of a compound dislcosed herein and/or combinations with other therapeutics are combined with genetic testing to determine whether that individual is a carrier of a mutant gene that is known to be correlated with certain diseases or conditions.

The compounds described herein and combination therapies can be administered before, during or after the occurrence of a disease or condition, and the timing of administering the composition containing a compound can vary. Thus, for example, the compounds can be used as a prophylactic and can be administered continuously to subjects with a propensity to develop conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, within the first 6 hours of the onset of the symptoms, or within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. A compound should be administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria. For example, the compound or a formulation containing the compound can be administered for at least 2 weeks, between about 1 month to about 5 years, or from about 1 month to about 3 years. Exemplary Therapeutic Agents for Use in Combination with a Menin or Menin-MLL inhibitor Compound

Where the subject is suffering from or at risk of suffering from an autoimmune disease, an inflammatory disease, or an allergy disease, a menin or menin-MLL inhibitor compound can be used in with one or more of the following therapeutic agents in any combination: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific irreversible inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

Where the subject is suffering from or at risk of suffering from a B-cell proliferative disorder (e.g., plasma cell myeloma), the subjected can be treated with a menin or menin-MLL inhibitor compound in any combination with one or more other anti-cancer agents. In some embodiments, one or more of the anti-cancer agents are proapoptotic agents. Examples of anti-cancer agents include, but are not limited to, any of the following: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™ Compounds that have the basic taxane skeleton as a common structure feature, have also been shown to have the ability to arrest cells in the G2-M phases due to stabilized microtubules and may be useful for treating cancer in combination with the compounds described herein.

Other anti-cancer agents that can be employed in combination with an menin or menin-MLL inhibitor compound include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rlL2), interferon α-2a; interferon α-2b; interferon α-n1; interferon α-n3; interferon β-la; interferon γ-lb; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with a menin or menin-MLL inhibitor compound include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis irreversible inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase irreversible inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase irreversible inhibitors; gemcitabine; glutathione irreversible inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin irreversible inhibitors; matrix metalloproteinase irreversible inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase irreversible inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome irreversible inhibitors; protein A-based immune modulator; protein kinase C inhibitor; protein kinase C irreversible inhibitors, microalgal; protein tyrosine phosphatase irreversible inhibitors; purine nucleoside phosphorylase irreversible inhibitors; purpurins; pyrazoloacridine; pyridoxylated hemoglobin polyoxyethylerie conjugate; raf antagonists; raltitrexed; ramosetron; ras farnesyl protein transferase irreversible inhibitors; ras irreversible inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction irreversible inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division irreversible inhibitors; stipiamide; stromelysin irreversible inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase irreversible inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentine; toremifene; totipotent stem cell factor; translation irreversible inhibitors; tretinoin; triacetyluridine; triciribine; trimetrexate; triptorelin; tropisetron; turosteride; tyrosine kinase irreversible inhibitors; tyrphostins; UBC irreversible inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with a menin or menin-MLL inhibitor compound include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products useful in combination with a menin or menin-MLL inhibitor compound include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination a menin or menin-MLL inhibitor compound include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to, folic acid analog (e.g., methotrexate), or pyrimidine analogs (e.g., fluorouracil, floxouridine, Cytarabine), purine analogs (e.g., mercaptopurine, thioguanine, pentostatin.

Examples of hormones and antagonists useful in combination with a menin or menin-MLL inhibitor compound include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with an a menin or menin-MLL inhibitor compound include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP—XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C(also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR- 182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39·HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR—OY-007 (National Health Research Institutes), and SSR-250411 (Sanofi).

Where the subject is suffering from or at risk of suffering from a thromboembolic disorder (e.g., stroke), the subject can be treated with a menin or menin-MLL inhibitor compound in any combination with one or more other anti-thromboembolic agents. Examples of anti-thromboembolic agents include, but are not limited any of the following: thrombolytic agents (e.g., alteplase anistreplase, streptokinase, urokinase, or tissue plasminogen activator), heparin, tinzaparin, warfarin, dabigatran (e.g., dabigatran etexilate), factor Xa irreversible inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIBR 1048.

Where the subject is suffering from or at risk of suffering from an autoimmune disease or an inflammatory disease, a menin or menin-MLL inhibitor compound can be used in with one or more of second therapeutic agents in any combination.

A menin or menin-MLL inhibitor compound can be used in with one or more of second therapeutic agents in any combination. In certain embodiments, the second agent is an alpha-glucosidase agonists. In certain embodiments, the second agent is miglitol or acarbose. In certain embodiments, the second agent is an amylin analog. In certain embodiments, the second agent is pramlintide. In certain embodiments, the second agent is dipeptidyl peptidase 4 inhibitor. In certain embodiments, the second agent is selected from sitagliptin, linagliptin, saxagliptin, and alogliptin. In certain embodiments, the second agent is an incretin mimetic. In certain embodiments, the second agent is selected from liraglutide, semaglutine, dulaglutide, tirzepatide, exenatide, albiglutide, and lixisenatide. In certain embodiments, the second agent is insulin. In certain embodiments, the second agent is selected from insulin, insulin degludec, insulin glargine, insulin detemir, insulin lispro, insulin isophane, insulin aspart, insulin glulisine, and insulin zinc. In certain embodiments, the second agent is a meglitinide. In certain embodiments, the second agent is selected from repaglinide and nateglinide. In certain embodiments, the second agent is teplizumab. In certain embodiments, the second agent is a non-sulfonylurea. In certain embodiments, the second agent is metformin. In certain embodiments, the second agent is an SGLT-2 inhibitor. In certain embodiments, the second agent is empagliflozin, canagliflozin, dapagliflozin, ertugliflozin, or bexagliflozin. In certain embodiments, the second agent is a sulfonylurea. In certain embodiments, the second agent is glimepiride, glipizide, tolazamide, tolbutamide, glyburide, or chlorpropamide. In certain embodiments, the second agent is a thiazolidinedione. In certain embodiments, the second agent is pioglitazone or rosiglitazone.

### Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease, disorder, or condition that would benefit by inhibition of menin, or in which menin is a mediator or contributor to the symptoms or cause.

For example, the container(s) can include one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit typically may include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

The examples below as well as throughout the application, the following abbreviations have the following meanings. If not defined, the terms have their generally accepted meanings.
aq aqueous
AIBN=2,2′-Azobis(2-methylpropionitrile)
Boc=tert-butyloxycarbonyl
t-BuOH=tertiary butanol
DCE 1,2-dichloroethane
DCM=dichloromethane
DIAD=diisopropyl azodicarboxylate
DIEA or DIPEA=N,N-diisopropylethylamine
DMAC=dimethylacetamide
DMAP=dimethylaminopyridine
DMF=dimethylformamide
DMSO=dimethylsulfoxide
ESI=electron spray ionization
EA=ethyl acetate
g=gram
HCl=hydrogen chloride
HPLC=high performance liquid chromatography
hr hour
¹H NMR=proton nuclear magnetic resonance
IPA=isopropyl alcohol
KOAc=potassium acetate
LC-MS=liquid chromatography mass spectroscopy
M molar
MeCN
MeOH=
acetonitrile
methanol
mg=milligram
min=minute
ml=milliliter
mM=millimolar
mmol=millimole
m.p.=melting point
MS=mass spectrometry
m/z=mass-to-charge ratio
N normal
NBS=N-bromosuccinimide
NIS═N-iodosuccinimide
nM=nanomolar
nm=nanometer
Pd(dppf)Cl₂═[1,1′-Bis(diphenylphosphino) ferrocene]dichloropalladium (II)
PE=petroleum ether
PyBOP=benzotriazol-1-yl-oxytripyrrolidinophosphonium hexafluorophosphate
quant.=quantitative
RP=reverse phase
rt or r.t.=room temperature
Sat.=saturated
TEA=triethylamine
TFA=trifluoroacetic acid
μL=microliter
μM=Micromolar

### Synthesis of Intermediates

### Synthesis of Intermediates 3A and 5A

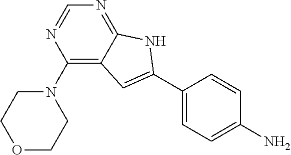 [SMILES: Nc1ccc(-c2cc3c(N4CCOCC4)ncnc3[nH]2)cc1]

### General Procedure for Preparation of Intermediate 3A

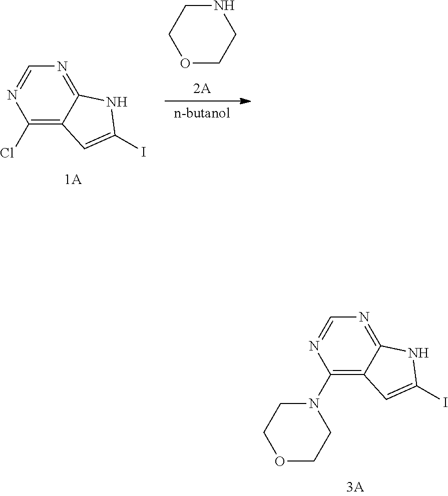 [SMILES: C1COCCN1 ; Clc1ncnc2[nH]c(I)cc12 ; Ic1cc2c(N3CCOCC3)ncnc2[nH]1]

A solution of morpholine (3.12 g, 35.7 mmol, 3.15 mL, 2 eq) and Intermediate 1A (5.00 g, 17.8 mmol, 1 eq) in n-butanol (25.0 mL) was heated at 100° C. for 12 h. The color of the solution became white. TLC (Dichloromethane/Methanol=10/1, R_{f}=0.60) indicated the starting material was consumed completely. The reaction mixture was diluted with H₂O (200.0 mL) and extracted with EtOAc (100.0 mL×3). The combined organic layers were washed with brine (100.0 mL), dried over Na₂SO₄, filtered, and concentrated under reduced pressure to give a residue. The compound was used for the next step without further purification. Intermediate 3A (5.08 g, 15.3 mmol, 86.0% yield) was obtained as a white solid

### General Procedure for Preparation of Intermediate 5A

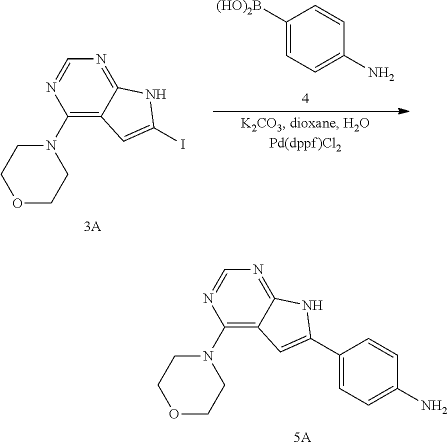 [SMILES: Ic1cc2c(N3CCOCC3)ncnc2[nH]1 ; Nc1ccc(B(O)O)cc1 ; Nc1ccc(-c2cc3c(N4CCOCC4)ncnc3[nH]2)cc1]

A solution of (4-aminophenyl) boronic acid (2.49 g, 18.1 mmol, 1.5 eq), Intermediate 3A (4.00 g, 12.1 mmol, 1 eq) and K₂CO₃ (10.0 g, 72.7 mmol, 6 eq) in dioxane (20.0 mL) and H₂O (4.00 mL) was degassed with argon 30 min. Cyclopentyl(diphenyl)phosphane; dichloropalladium; iron (886.5 mg, 1.21 mmol, 0.1 eq) was added to the reactor. The mixture was refluxed at 100° C. for 12 h. The color of the solution become black. TLC(Dichloromethane/Methanol=10/1, R_{f}=0.57) indicated the starting material was consumed completely. The reaction mixture was diluted with H₂O (300.0 mL) and extracted with EtOAc (300.0 mL×3). The combined organic layers were washed with brine (300.0 mL), dried over Na₂SO₄, filtered and concentrated under reduced pressure to give a residue. The residue was purified by column chromatography (SiO₂, Petroleum ether/Ethyl acetate=50/1 to 5/1) Intermediate 5A (3.00 g, 10.1 mmol, 83.8% yield) was obtained as a white solid.

### General Procedure for Preparation of

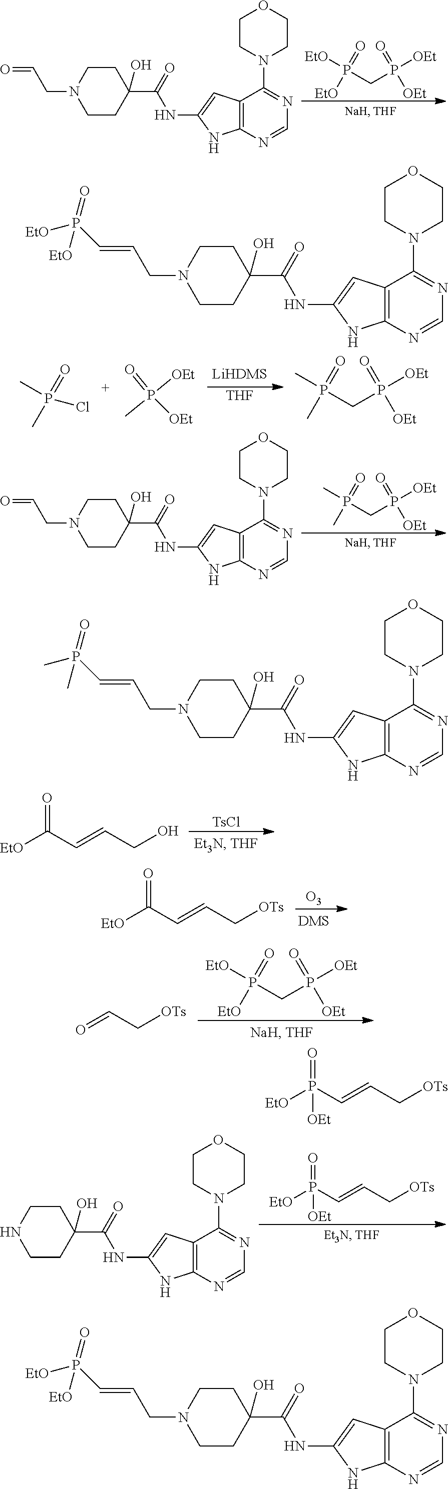

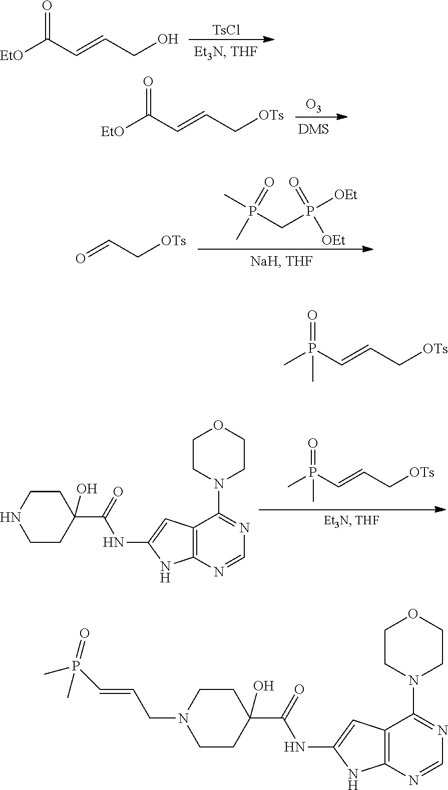

### Intermediate Acid

### Synthesis of (R)-4-((3-((tert-butoxycarbonyl)amino)piperidin-1-yl)methyl)picolinic acid Lithium salt (2)

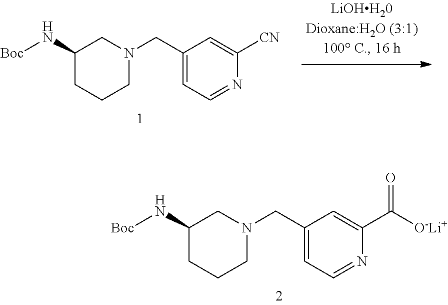

To a stirred solution of tert-butyl N-[(3R)-1-[(2-cyanopyridin-4-yl)methyl]piperidin-3-yl]carbamate 1 (4.00 g, 12.6 mmol) in 1,4-dioxane (30 mL) and water (10 mL) was added LiOH·H₂O (6.35 g, 151.2 mmol) portion wise at room temperature. The reaction mixture was stirred at 100° C. for 16 h. After completion of reaction (TLC monitoring), solvent was concentrated under reduced pressure to get crude residue. Crude was dissolved in 25% IPA: CHCl₃ (200 mL) and solid inorganic salt was filtered out. The filtrated was concentrated under reduced pressure to get desired product as an off-white solid 2 (4.5 g, quantitative). The acid intermediate was used as Li′ salt for next reaction.

¹H NMR (400 MHz, DMSO d₆): δ 8.39-8.37 (d, J=4.8 Hz, 1H), 7.90 (s, 1H), 7.38-7.37 (d, J=4.4 Hz, 1H), 6.74-6.72 (d, J=8 Hz, 1H), 3.57 (s, 2H), 2.74-2.71 (m, 1H), 2.57-2.49 (m, 1H), 1.91-1.46 (m, 5H), 1.35 (s, 9H), 1.28 (m, 1H) and 1.10 (m, 1H). LCMS=[M+H]⁺: 336.14, Purity=95%.

### Intermediate Alcohol

### Synthesis of 1-(tert-butyl) 4-methyl 4-(hydroxymethyl)piperidine-1,4-dicarboxylate (2)

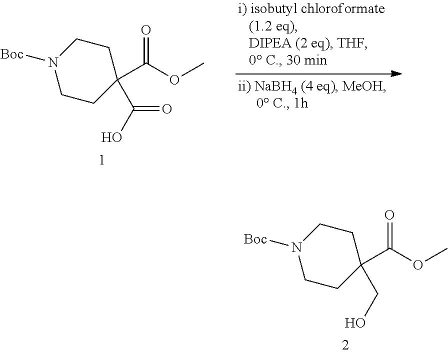 [SMILES: COC(=O)C1(C(=O)O)CCN(C(=O)OC(C)(C)C)CC1 ; COC(=O)C1(CO)CCN(C(=O)OC(C)(C)C)CC1]

To an ice-cold stirred solution of 1-(tert-butoxycarbonyl)-4-(methoxycarbonyl)piperidine-4-carboxylic acid 1 (1 g, 3.48 mmol) and DIPEA (1.2 mL, 2 eq., 6.96 mmol) in THF (10 mL) was added isobutyl chloroformate (0.54 mL, 1.2 eq., 4.17 mmol) in drop wise manner. The resulting reaction mixture was stirred for next 30 minutes. After confirmation of mixed anhydride (by TLC) formation, added MeOH (3.0 mL), followed by portion wise addition of NaBH₄ (527 mg, 4 eq., 13.9 mmol) at the same temperature. The resulting reaction mixture was stirred for another one hour at room temperature. After completion of the reaction (monitoring by TLC), the reaction mixture was quenched with sat. NaHCO₃ and solvent was partially evaporated under reduced pressure. The residue was partitioned between EtOAc and water. The combined organic layer was washed with brine, dried over Na₂SO₄, filtered and concentrated to dryness. The crude was purified over flash column chromatography using eluent (2-7% MeOH in DCM) to obtain 1-(tert-butyl) 4-methyl 4-(hydroxymethyl)piperidine-1,4-dicarboxylate 2 (750 mg, 84%) as oil.

¹H-NMR (400 MHz, DMSO-d₆): δ 4.93 (t, J=6 Hz, 1H), 3.71-3.67 (m, 1H), 3.62 (s, 3H), 3.41 (d, J=5.6 Hz, 2H), 2.83 (brs, 2H), 1.90-1.86 (m, 2H), 1.37 (s, 9H), 1.29-1.35 (m, 2H).

### Intermediate X: (R)—N-(4-(4-morpholino-7H-pyrrolo[2,3-d]pyrimidin-6-yl)phenyl)-4-((3-(2-(piperazin-1-ylmethyl)acrylamido)piperidin-1-yl)methyl)picolinamide

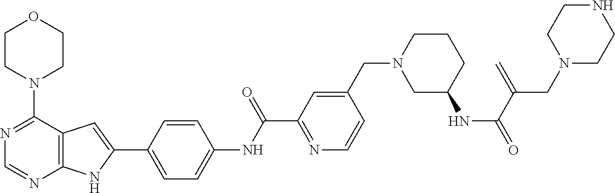 [SMILES: C=C(CN1CCNCC1)C(=O)N[C@@H]1CCCN(Cc2ccnc(C(=O)Nc3ccc(-c4cc5c(N6CCOCC6)ncnc5[nH]4)cc3)c2)C1]

Step 1:2-((4-(tert-Butoxycarbnyl)piperazin-1-yl)methyl)acrylic acid

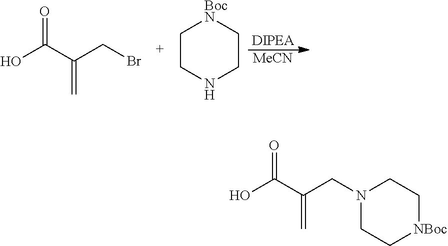

A solution of 2-(bromomethyl)prop-2-enoic acid (425 mg, 2.58 mmol), tert-butyl piperazine-1-carboxylate (624 mg, 3.35 mmol) and N,N-diisopropylethylamine (1.3 mL, 7.73 mmol) in acetonitrile (6 mL) was stirred at 35° C. for 20 hours, providing a solution of 2-((4-(tert-butoxycarbnyl)piperazin-1 -yl)methyl)acrylic acid (696 mg, 2.57 mmol, 99%) in acetonitrile. The solution (0.35 M) was used in the next step without workup.

LCMS=[M+H]⁺: 271.20.

Step 2: tert-butyl(R)-4-(2-((1-((2-((4-(4-morpholino-7H-pyrrolo[2,3-d]pyrimidin-6-yl)phenyl)carbamoyl)pyridin-4-yl)methyl)piperidin-3-yl)carbamoyl) allyl)piperazine-1-carboxylate

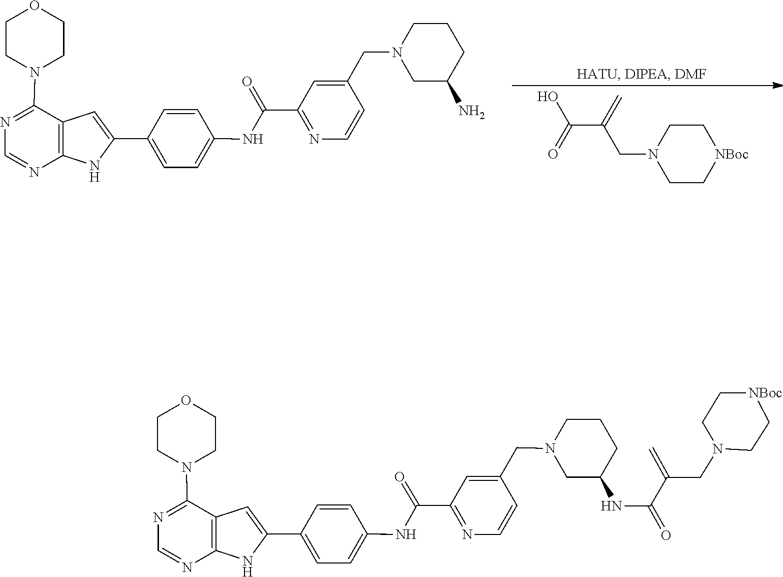 [SMILES: N[C@@H]1CCCN(Cc2ccnc(C(=O)Nc3ccc(-c4cc5c(N6CCOCC6)ncnc5[nH]4)cc3)c2)C1 ; C=C(CN1CCN(C(=O)OC(C)(C)C)CC1)C(=O)O ; C=C(CN1CCN(C(=O)OC(C)(C)C)CC1)C(=O)N[C@@H]1CCCN(Cc2ccnc(C(=O)Nc3ccc(-c4cc5c(N6CCOCC6)ncnc5[nH]4)cc3)c2)C1]

A solution of 2-((4-(tert-butoxycarbnyl)piperazin-1-yl)methyl)acrylic acid (0.41 mL, 0.145 mmol), (0.35 M, reaction mixture without workup) was added to a solution of ( ) (7-azabenzotriazol-1-yl)-N,N,N′,N′-tetramethyluronium hexafluorophosphate (60 mg, 0.157 mmol) and N,N-diisopropylethylamine (0.13 mL, 0.723 mmol) in N,N-dimethylformamide (3 mL). After 5 minutes (R)-4-((3-aminopiperidin-1-yl)methyl)-N-(4-(4-morpholino-7H-pyrrolo[2,3-d]pyrimidin-6-yl)phenyl)picolinamide trihydrochloride (75 mg, 0.121 mmol) was added. After 1 hour the mixture was diluted with ethyl acetate (30 mL) and washed with 5% lithium chloride (3×10 mL), saturated sodium bicarbonate (10 mL), saturated ammonium chloride (10 mL). The organic phase was dried over sodium sulfate and the solvent was removed under reduced pressure. The residue was subjected to flash chromatography (0-100% 20% methanol in ethyl acetate/hexanes). The fractions containing product were combined and the solvent was removed under reduced pressure, providing tert-butyl(R)-4-(2-((1-((2-((4-(4-morpholino-7H-pyrrolo[2,3-d]pyrimidin-6-yl)phenyl)carbamoyl)pyridin-4-yl)methyl)piperidin-3-yl)carbamoyl) allyl)piperazine-1-carboxylate (51 mg, 0.0667 mmol, 55%).

LCMS=[M+H]⁺: 765.50.

Step 3: (R)—N-(4-(4-morpholino-7H-pyrrolo[2,3-d]pyrimidin-6-yl)phenyl)-4-((3-(2-(piperazin-1-ylmethyl)acrylamido)piperidin-1-yl)methyl)picolinamide

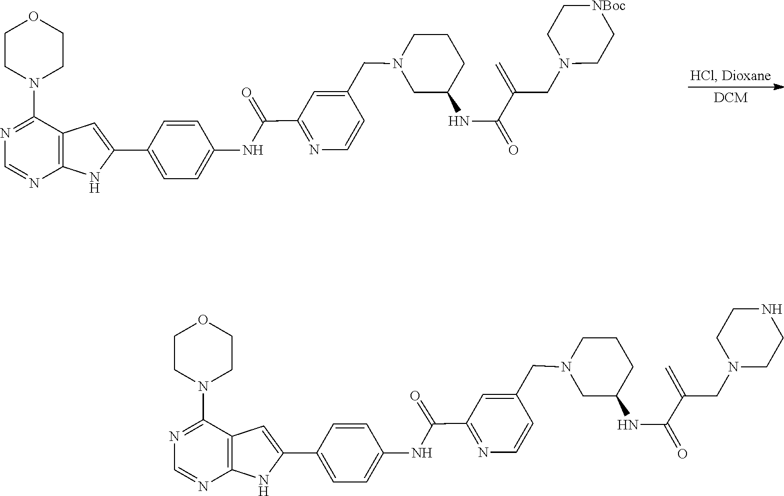 [SMILES: C=C(CN1CCN(C(=O)OC(C)(C)C)CC1)C(=O)N[C@@H]1CCCN(Cc2ccnc(C(=O)Nc3ccc(-c4cc5c(N6CCOCC6)ncnc5[nH]4)cc3)c2)C1 ; C=C(CN1CCNCC1)C(=O)N[C@@H]1CCCN(Cc2ccnc(C(=O)Nc3ccc(-c4cc5c(N6CCOCC6)ncnc5[nH]4)cc3)c2)C1]

A solution of hydrochloric acid in 1,4-dioxane (4N, 0.50 mL, 2.00 mmol) was added to a solution of tert-butyl(R)-4-(2-((1-((2-((4-(4-morpholino-7H-pyrrolo[2,3-d]pyrimidin-6-yl)phenyl)carbamoyl)pyridin-4-yl)methyl)piperidin-3-yl)carbamoyl) allyl)piperazine-1-carboxylate (51 mg, 0.0667 mmol) in dichloromethane (5 mL). After 40 minutes the volatiles were removed under reduced pressure, providing (R)—N-(4-(4 -morpholino-7H-pyrrolo[2,3-d]pyrimidin-6-yl)phenyl)-4-((3-(2-(piperazin-1-ylmethyl)acrylamido)piperidin-1-yl)methyl)picolinamide trihydrochloride (51 mg, 0.0659 mmol 98%).

LCMS=[M+H]⁺: 665.40.

The compounds ID 201-506 were prepared according to the methods described in WO2023235618.

### Example 1

### Compound 601

### Preparation of (R)-4-((3-(4-(dimethylamino)but-2-ynamido)piperidin-1-yl)methyl)-N-(4-(4-morpholino-7H-pyrrolo[2,3-d]pyrimidin-6-yl)phenyl)picolinamide (Compound 601)

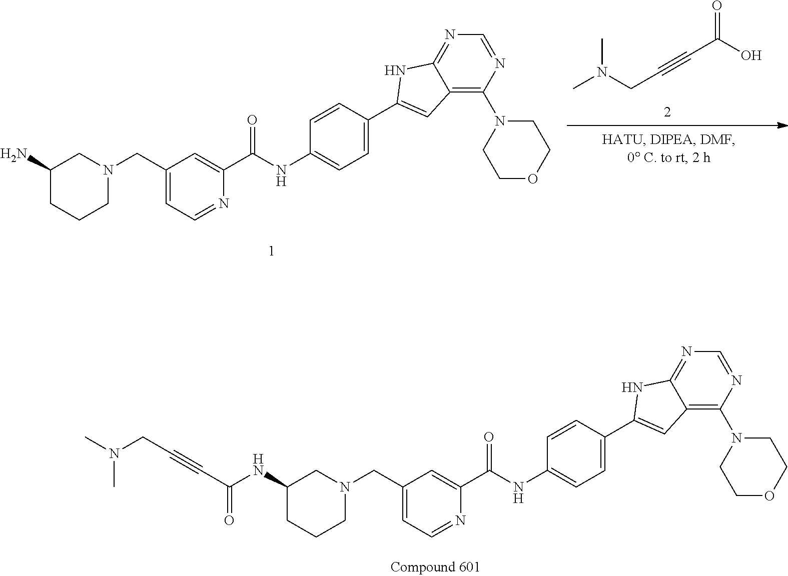

To an ice cold stirred solution of N-[p-(4-morpholino-1H-1,5,7-triazainden-2-yl)phenyl]-4-{[(R)-3-amino-1-piperidyl]methyl}-2-pyridinecarboxamide 1 (300 mg, 0.585 mmol) and 4-(dimethylamino)-2-butynoic acid 2 (112 mg, 0.878 mmol) in DMF (10 mL) were added DIPEA (0.3 mL, 1.76 mmol) and HATU (334 mg, 0.878 mmol) sequentially under N₂ atmosphere. The reaction mixture was stirred at RT for 2 h. After completion of reaction (monitoring by TLC and LCMS), the reaction mass was poured into ice cold water (50 mL) and extracted with 25% IPA in CHCl₃ (3×50 mL). The combined organic layer was dried over Na₂SO₄, filtered and concentrated under reduced pressure. The crude was purified by RP-HPLC [5-95% 0.1% TFA in Water/ACN; Column: Atlantis T3 (19*250) 10 μ] to get the desired Compound 601 (90 mg, 25%).

¹H NMR (400 MHz, DMSO-d₆): δ 12.41 (s, 1H), 10.84 (s, 1H), 10.33-10.04 (m, 1H), 8.85 (d, J=4.8 Hz, 2H), 8.35 (br, s, 1H), 8.23 (d, J=8.8 H), 7.93 (d, J=8.8 Hz, 2H), 7.76 (br, s, 1H), 7.22 (s, 1H), 4.54 (s, 2H), 4.29 (s, 2H), 3.92-3.90 (m, 4H), 3.78-3.50 (m, 9H), 2.83 (s, 6H), 1.87 (m, 2H), 1.67-1.64 (m, 1H), 1.41-1.39 (m, 1H).

LCMS=[M+H]⁺: 622.45; Purity=99.69%.

### Example 2

### Compound 602

### Preparation of (R, E)-4-((3-(4-(dimethylamino)but-2-enamido)-3-methylpiperidin-1-yl)methyl)-N-(4-(4-morpholino-7H-pyrrolo[2,3-d]pyrimidin-6-yl)phenyl)picolinamide (Compound 602)

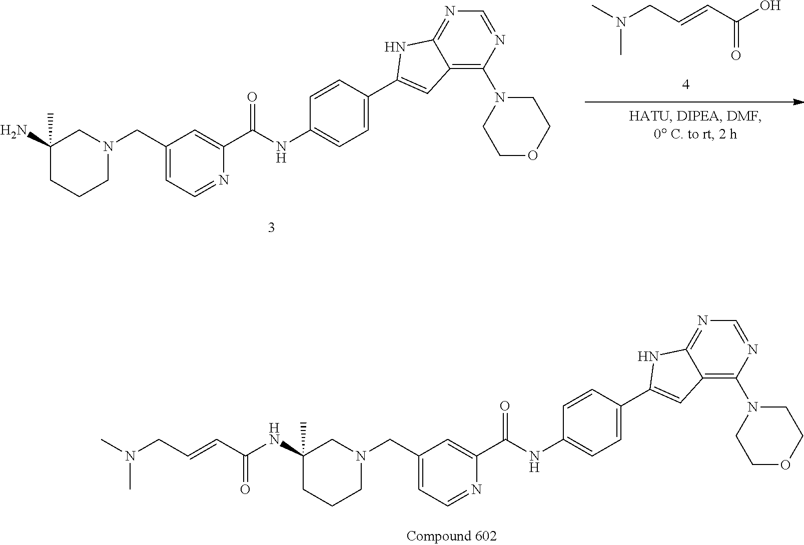

To a stirred solution of (R)-4-((3-amino-3-methylpiperidin-1-yl)methyl)-N-(4-(4-morpholino-7H-pyrrolo[2,3-d]pyrimidin-6-yl)phenyl)picolinamide 3 (300 mg, 0.570 mmol) and (E)-4-(dimethylamino)-2-butenoic acid 4 (95.6 mg, 0.741 mmol) in DMF (5 mL) were added DIPEA (0.3 mL, 1.71 mmol) and HATU (433 mg, 1.14 mmol) sequentially at 0° C. and the reaction mixture was stirred at RT for 2 h. After completion of reaction (TLC and LCMS monitoring), the reaction mixture was poured into ice cold water (50 mL) and extracted with 25% IPA in CHCl₃ (3×50 mL). The combined organic layer was washed with ice cold brine solution (2×50 mL), dried over Na₂SO₄, filtered and concentrated under reduced pressure. The crude was purified by RP-HPLC [5-95% 5 mM ammonium bicarbonate in water/ACN; Column: X Bridge C8 (19*250) 5μ] to get Compound 602 (43 mg, 12%).

¹H NMR (400 MHz, DMSO-d₆): δ 12.21 (s, 1H), 10.74 (s, 1H), 8.65 (d, J=4.8 Hz, 1H), 8.18 (s, 1H), 8.12 (s, 1H), 8.01-7.99 (m, 2H), 7.92-7.90 (m, 2H), 7.61 (d, J=4.8 Hz, 1H), 7.37 (s, 1H), 7.16 (s, 1H), 6.49-6.42 (m, 1H), 6.15-6.12 (m, 1H), 3.89-3.87 (m, 4H), 3.76-3.74 (m, 4H), 3.68-3.64 (m, 1H), 3.57-3.53 (m, 1H), 2.95 (d, J=6 Hz, 2H), 2.72-2.69 (m, 1H), 2.43-2.39 (m, 2H), 2.27-2.23 (m, 1H), 2.12 (s, 6H), 1.93-1.90 (m, 1H), 1.59-1.53 (m, 2H), 1.37-1.34 (m, 4H).

LCMS=[M+H]⁺: 638.45; Purity=99.23%.

### Example 3

### Compound 603

### Preparation of (R,E)-4-((3-(4-(3,3-difluoroazetidin-1-yl)but-2-enamido)-3-methylpiperidin-1-yl)methyl)-N-(4-(4-morpholino-7H-pyrrolo[2,3-d]pyrimidin-6-yl)phenyl)picolinamide (Compound 603)

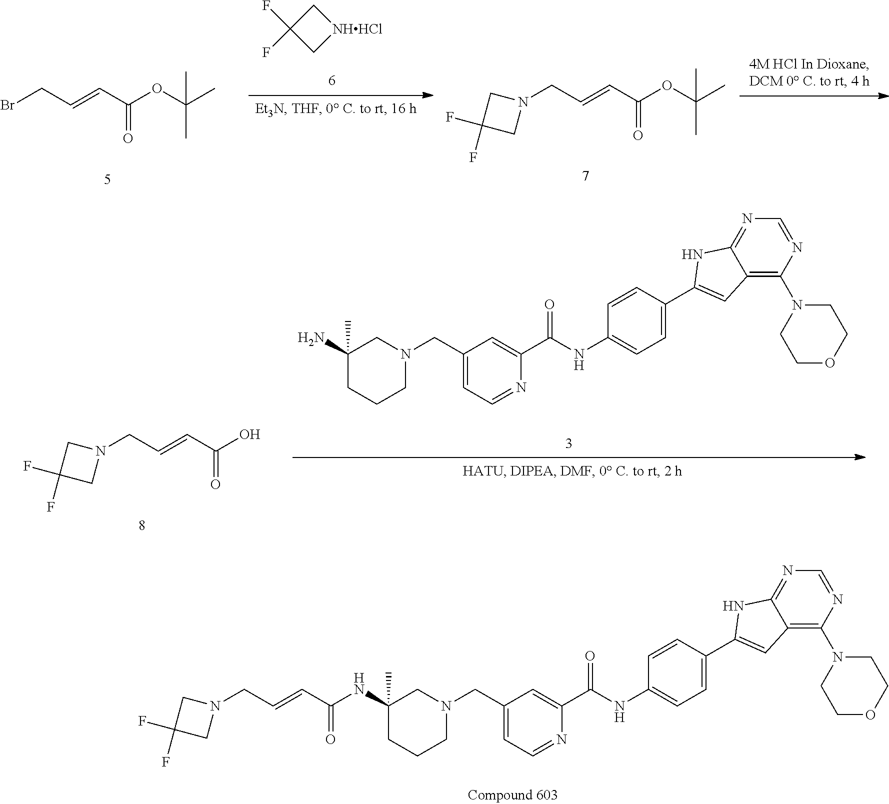

### Synthesis of tert-butyl(E)-4-(3,3-difluoroazetidin-1-yl)but-2-enoate (7)

To a stirred solution of tert-butyl(E)-4-bromobut-2-enoate 5 (1.5 g, 7.24 mmol) and 3,3-difluoroazetidine hydrogen chloride 6 (1.13 g, 8.69 mmol) in THF (15 mL) was added Et₃N(3.01 mL, 21.7 mmol) at 0° C. and the reaction mixture was stirred at RT for 16 h. After completion of reaction (TLC monitoring), the reaction mass was poured into ice cold water (50 mL) and extracted with DCM (3×50 mL). The combined organic layer was washed with brine solution (2×50 mL), dried over Na₂SO₄, filtered and concentrated under reduced pressure. The crude was purified by column chromatography using 20-25% EtOAc in heptane to get the title compound as a light yellow liquid 7 (800 mg, 47%).

LCMS=[M+H]⁺: 234.12; Purity=98%.

### Synthesis of (E)-4-(3,3-difluoroazetidin-1-yl)but-2-enoic acid (8)

To a stirred solution of tert-butyl(E)-4-(3,3-difluoroazetidin-1-yl)but-2-enoate 7 (750 mg, 3.22 mmol) in DCM (5 mL) was added 4N HCl in 1,4-dioxane (5 mL) at 0° C. and the reaction was stirred at RT for 4 h. After completion of reaction (TLC and LCMS monitoring), the reaction mixture was concentrated under reduced pressure. The crude was triturated with Et₂O and dried under reduced pressure to get the title compound as a brown solid 8 (700 mg; HCl salt), crude was used in next step directly without further purification. LCMS=[M+H]⁺: 178.06; Purity=95%.

### Synthesis of (R,E)-4-((3-(4-(3,3-difluoroazetidin-1-yl)but-2-enamido)-3-methylpiperidin-1-yl)methyl)-N-(4-(4-morpholino-7H-pyrrolo[2,3-d]pyrimidin-6-yl)phenyl)picolinamide (Compound 603)

To a stirred solution of N-[p-(4-morpholino-1H-1,5,7-triazainden-2-yl)phenyl]-4-{[(R)-3-amino-3-methyl-1-piperidyl]methyl}-2-pyridinecarboxamide 3 (300 mg, 0.570 mmol) and (E)-4-(3,3-difluoro-1-azetidinyl)-2-butenoic acid 8 (131 mg, 0.741 mmol) in DMF (5 mL) were added DIPEA (0.3 mL, 1.71 mmol) and HATU (433 mg, 1.14 mmol) sequentially at 0° C. and the reaction mixture was stirred at RT for 2 h. After completion of reaction (TLC and LCMS monitoring), the reaction mixture was poured into ice cold water (50 mL) and extracted with 25% IPA in CHCl₃ (3×50 mL). The combined organic layer was washed with ice cold brine solution (2×50 mL), dried over Na₂SO₄, filtered and concentrated under reduced pressure. The crude was purified by RP-HPLC [5-95% 5 mM ammonium bicarbonate in water/ACN; Column: X Bridge C8 (19*250) 5μ] to get Compound 603 (40 mg, 10%).

¹H NMR (400 MHz, DMSO-d₆): δ 12.20 (s, 1H), 10.73 (s, 1H), 8.66 (d, J=4.9 Hz, 1H), 8.18 (s, 1H), 8.12 (s, 1H), 8.01-7.99 (m, 2H), 7.91-7.89 (m, 2H), 7.60 (d, J=4.9 Hz, 1H), 7.39 (s, 1H), 7.15 (d, J=1.5 Hz, 1H), 6.44-6.38 (m, 1H), 6.15-6.11 (m, 1H), 3.89-3.87 (m, 4H), 3.76-3.74 (m, 4H), 3.68-3.53 (m, 6H), 3.33-3.28 (m, 2H), 2.69 (s, 1H), 2.42-2.37 (m, 2H), 2.28-2.25 (m, 1H), 1.93-1.88 (m, 1H), 1.62-1.48 (m, 2H), 1.42-1.38 (m, 1H), 1.33 (s, 3H).

LCMS=[M+H]⁺: 686.47; Purity=99.93%.

### Example 4

### Compound 604

### Preparation of (R)-4-((3-(4-(dimethylamino)but-2-ynamido)-3-methylpiperidin-1-yl)methyl)-N-(4-(4-morpholino-7H-pyrrolo[2,3-d]pyrimidin-6-yl)phenyl)picolinamideynamide (Compound 604)

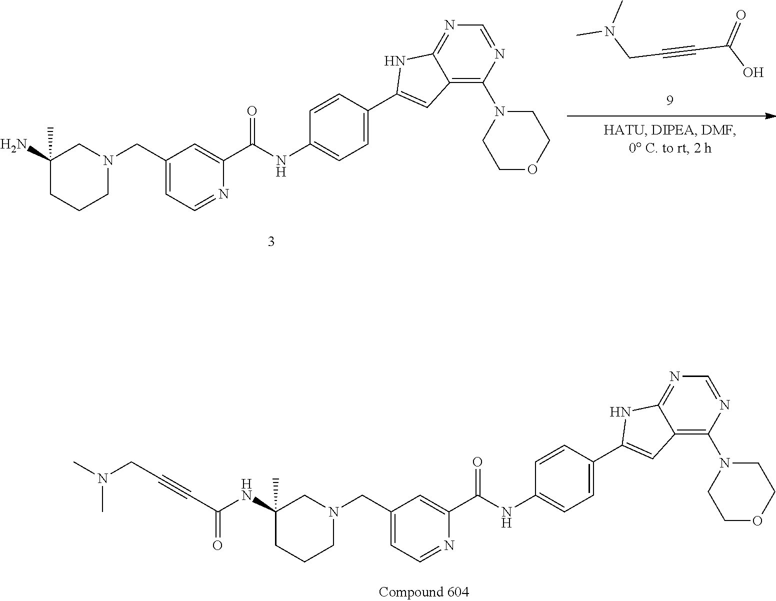

To a stirred solution of N-[p-(4-morpholino-1H-1,5,7-triazainden-2-yl)phenyl]-4-{[(R)-3-amino-3-methyl-1-piperidyl]methyl}-2-pyridinecarboxamide 3 (300 mg, 0.570 mmol) and 4-(dimethylamino)-2-butynoic acid 9 (94.2 mg, 0.741 mmol) in DMF (5 mL) were added DIPEA (0.3 mL, 1.71 mmol) and HATU (433 mg, 1.14 mmol) sequentially at 0° C. The resulting reaction mixture was stirred at RT for 2 h. After completion of reaction (TLC and LCMS monitoring), the reaction mixture was poured into ice cold water (50 mL) and extracted with 25% IPA in CHCl₃ (3×50 mL). The combined organic layer was washed with ice cold brine solution (2×50 mL), dried over Na₂SO₄, filtered and concentrated under reduced pressure. The crude was purified by RP-HPLC [5-95% 5 mM ammonium bicarbonate in water/ACN; Column: X Bridge C8 (19*250) 5μ] to get Compound 604 (25 mg, 7%).

¹H NMR (400 MHz, DMSO-d₆): δ 12.20 (s, 1H), 10.74 (s, 1H), 8.66 (d, J=4.8 Hz, 1H), 8.18 (s, 1H), 8.13 (s, 1H), 8.01-7.99 (m, 2H), 7.92-7.90 (m, 2H), 7.81 (brs, 1H), 7.60 (d, J=4.8 Hz, 1H), 7.16 (d, J=1.2 Hz, 1H), 3.89-3.87 (m, 4H), 3.76-3.74 (m, 4H), 3.69-3.65 (m, 1H), 3.57-3.53 (m, 1H), 3.36 (s, 2H), 2.72-2.70 (m, 1H), 2.41-2.40 (m, 1H), 2.28-2.25 (m, 2H), 2.20 (s, 6H), 1.92-1.89 (m, 1H), 1.61-1.46 (m, 2H), 1.38-1.31 (m, 1H), 1.23 (s, 3H).

LCMS=[M+H]⁺: 637.47; Purity=99.87%.

### Example 5

### Compound 605

### Preparation of N-(4-(4-(3-oxa-8-azabicyclo[3.2.1]octan-8-yl)-7H-pyrrolo[2,3-d]pyrimidin-6-yl)phenyl)-4-(((R)-3-acrylamido-3-methylpiperidin-1-yl)methyl)picolinamide (Compound 605)

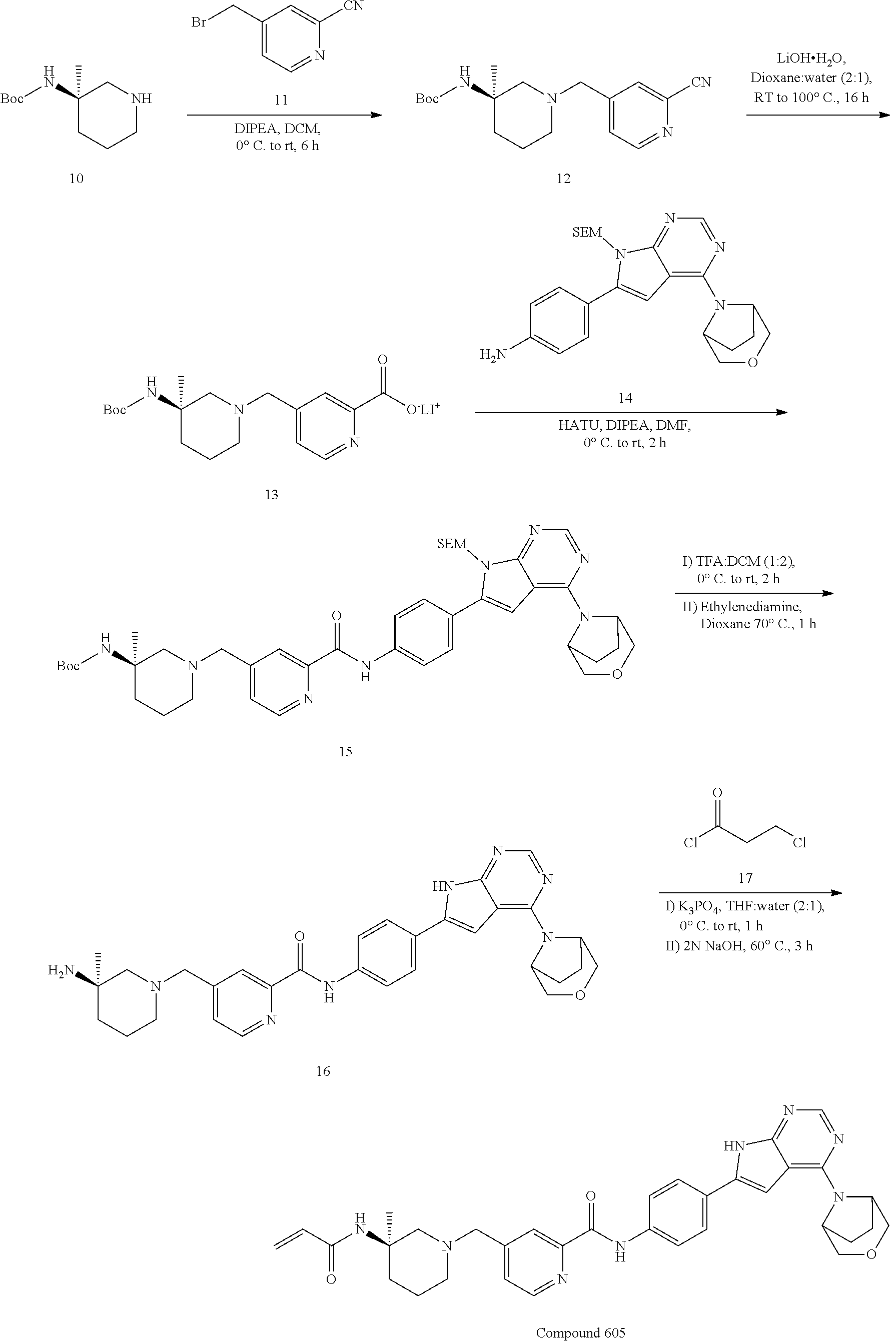

### Synthesis of tert-butyl(R)-(1-((2-cyanopyridin-4-yl)methyl)-3-methylpipcridin-3-yl)carbamate (12)

To a stirred solution of (R)-3-methyl-3-piperidylamino-tert-butylformylate 10 (400 mg, 1.87 mmol) and 4-(bromomethyl)-2-pyridinecarbonitrile 11 (736 mg, 3.73 mmol) in DCM (15 mL) was added DIPEA (0.965 mL, 5.6 mmol) at 0° C. and stirred at RT for 6 h. After completion of reaction (TLC and LCMS monitoring), the reaction mixture was diluted with water (50 mL) and extracted with DCM (3×100 mL). The combined organic layer was dried over Na₂SO₄, filtered and concentrated under reduced pressure. The crude was purified by column chromatography using eluents 20-25% EtOAc in heptane to get the title compound as a brown oil 12 (600 mg, 97%).

LCMS=[M+H]⁺: 331.29; Purity=97%.

### Synthesis of lithium (R)-4-((3-((tert-butoxycarbonyl)amino)-3-methylpiperidin-1-yl)methyl)picolinate (13)

To a stirred solution of tert-butyl(R)-(1-((2-cyanopyridin-4-yl)methyl)-3-methylpiperidin-3-yl)carbamate 12 (600 mg, 1.82 mmol) in 1,4-dioxane (6 mL) was added LiOH·H₂O (914 mg, 21.8 mmol) dissolved in water (3 mL) at RT. The resulting reaction mixture was stirred at 100° C. for 16 h. After completion of reaction (TLC and LCMS monitoring), the reaction mixture was concentrated under reduced pressure. The crude was dissolved in 25% IPA in CHCl₃ (50 mL) and stirred for 15 min at RT. The reaction mixture was filtered through sintered funnel and filtrate was concentrated under reduced pressure to get the title compound as a light yellow sticky solid 13 (550 mg; as Li salt), crude which was used in next reaction directly without further purification.

LCMS=[M+H]⁺: 350.37; Purity=91%.

### Synthesis of tert-butyl((3R)-1-((2-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)carbamoyl)pyridin-4-yl)methyl)-3-methylpiperidin-3-yl)carbamate (15)

To a stirred solution of lithium 4-{[(R)-3-(tert-butoxycarbonylamino)-3-methyl-1-piperidyl]methyl}-2-pyridinecarboxylate 13 (300 mg, 0.844 mmol) and p-[4-(3-oxa-8-azabicyclo[3.2.1]oct-8-yl)-1-{[2-(trimethylsilyl)ethoxy]methyl}-1H-1,5,7-triazainden-2-yl]aniline 14 (381 mg, 0.844 mmol) in DMF (6 mL) were added DIPEA (0.43 mL, 2.53 mmol) and HATU (642 mg, 1.69 mmol) sequentially at 0° C. and the reaction mixture was stirred at RT for 2 h. After completion of reaction (TLC and LCMS monitoring), the reaction mixture was poured into ice cold water (50 mL) and the solid precipitate was filtered through sintered funnel, washed with Et₂O and dried under vacuum. The crude was purified by flash chromatography (silica gel 100-200 M, 12 g SNAP) using 45-50% EtOAc in heptane to get the title compound as a yellow solid 15 (350 mg, 53%).

LCMS=[M+H]⁺: 784.47; Purity=80%.

### Synthesis of N-(4-(4-(3-oxa-8-azabicyclo[3.2.1]octan-8-yl)-7H-pyrrolo[2,3-d]pyrimidin-6-yl)phenyl)-4-(((R)-3-amino-3-methylpiperidin-1-yl)methyl)picolinamide (16)

To a stirred solution of 2-[p-(4-{[(R)-3-(tert-butoxycarbonylamino)-3-methyl-1-piperidyl]methyl}-2-pyridylcarbonylamino)phenyl]-4-(3-oxa-8-azabicyclo[3.2.1]oct-8-yl)-1-{[2-(trimethylsilyl)ethoxy]methyl}- 1H-1,5,7-triazaindene 15 (350 mg, 0.447 mmol) in DCM (6 mL) was added TFA (3 mL) dropwise at 0° C. and the reaction mixture was stirred at RT for 2 h. After completion of reaction (TLC monitoring), the reaction mixture was concentrated under reduced pressure. The crude was dissolved in 1,4-dioxane (5 mL) and 1,2-ethanediamine (0.1 mL, 1.34 mmol) was added at RT and the reaction mixture was stirred at 70° C. for 1 h. After completion of reaction (LCMS monitoring), the reaction mixture was diluted with 25% IPA in CHCl₃ (100 mL) and washed with ice cold water (2×50 mL). The organic layer was dried over Na₂SO₄, filtered and concentrated under reduced pressure to get the title compound as a white solid 16 (250 mg), crude which was used for next reaction as such.

LCMS=[M+H]⁺: 553.54; Purity=77%.

### Synthesis of N-(4-(4-(3-oxa-8-azabicyclo[3.2.1]octan-8-yl)-7H-pyrrolo[2,3-d]pyrimidin-6-yl)phenyl)-4-(((R)-3-acrylamido-3-methylpiperidin-1-yl)methyl)picolinamide (Compound 605)

To a stirred solution of N-(p-{4-(3-oxa-8-azabicyclo[3.2.1]oct-8-yl)-1H-1,5,7-triazainden-2-yl}phenyl)-4-{[(R)-3-amino-3-methyl-1-piperidyl]methyl}-2-pyridinecarboxamide 16 (250 mg, 0.452 mmol) in THF (5 mL) was added K₃PO₄ (192 mg, 0.905 mmol) dissolved in water (2.5 mL) at 0° C. and stirred for 5 min. Then, 3-chloropropionyl chloride 17 (68.9 mg, 0.543 mmol) was added to the reaction at 0° C. and stirred at RT for 1 h. After completion of SM (monitoring by TLC), 2N aq. NaOH (2.5 mL, 5.43 mmol) was added dropwise at 0° C. and stirred at 60° C. for 3 h. After complete elimination of chloro (LCMS monitoring), the resulting reaction mixture was poured into ice cold water (50 mL) and extracted with 2-MeTHF (3×50 mL). The combined organic layer was dried over Na₂SO₄, filtered and concentrated under reduced pressure. The crude was purified by flash column chromatography (silica gel 100-200 M, 12 g SNAP) using 5-6% MeOH in DCM to get Compound 605 (41 mg, 15%).

¹H NMR (400 MHz, DMSO-d₆): δ 12.18 (s, 1H), 10.73 (s, 1H), 8.66 (d, J=4.8 Hz, 1H), 8.17 (s, 1H), 8.12 (s, 1H), 8.00-7.98 (m, 2H), 7.93-7.91 (m, 2H), 7.61 (d, J=4.4 Hz, 1H), 7.44 (s, 1H), 7.12 (s, 1H), 6.36-6.30 (m, 1H), 6.01 (dd, J=17.2 Hz, 2 Hz, 1H), 5.51 (dd, J=10 Hz, 2 Hz, 1H), 4.92 (s, 2H), 3.72-3.54 (m, 6H), 2.71-2.64 (m, 1H), 2.48-2.35 (m, 2H), 2.30-2.25 (m, 1H), 2.07-1.92 (m, 5H), 1.64-1.48 (m, 2H), 1.42-1.39 (m, 1H), 1.31 (s, 3H).

LCMS=[M+H]: 607.48; Purity=95.06%.

### Example 6

### Compound 606

### Preparation of 5-acryloyl-N-(4-(4-morpholino-7H-pyrrolo[2,3-d]pyrimidin-6-yl)phenyl)octahydro-2H-pyrrolo[3,4-c]pyridine-2-carboxamide (Compound 606)

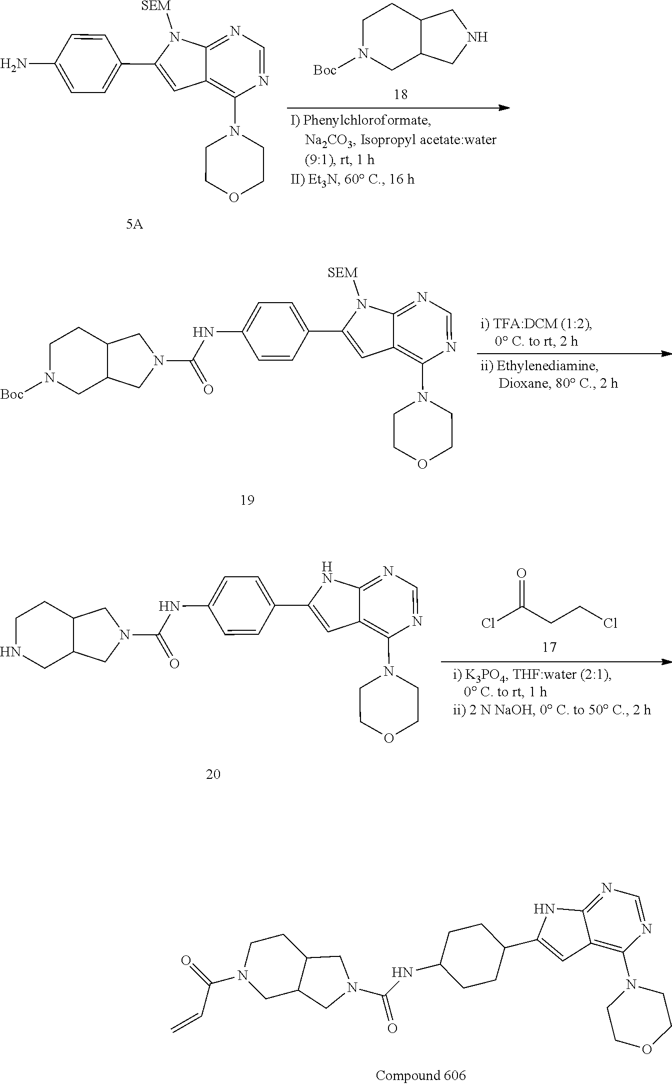 [SMILES: C[Si](C)(C)CCOCn1c(-c2ccc(N)cc2)cc2c(N3CCOCC3)ncnc21 ; CC(C)(C)OC(=O)N1CCC2CNCC2C1 ; CC(C)(C)OC(=O)N1CCC2CN(C(=O)Nc3ccc(-c4cc5c(N6CCOCC6)ncnc5n4COCC[Si](C)(C)C)cc3)CC2C1 ; O=C(Nc1ccc(-c2cc3c(N4CCOCC4)ncnc3[nH]2)cc1)N1CC2CCNCC2C1 ; O=C(Cl)CCCl ; C=CC(=O)N1CCC2CN(C(=O)NC3CCC(c4cc5c(N6CCOCC6)ncnc5[nH]4)CC3)CC2C1]

### Synthesis of tert-butyl 2-((4-(4-morpholino-7-((2-(trimethylsilyl)ethoxy)methyl)-7H-pyrrolo[2,3-d]pyrimidin-6-yl)phenyl)carbamoyl)octahydro-5H-pyrrolo[3,4-c]pyridine-5-carboxylate (19)

To a stirred solution of p-(4-morpholino-1-{[2-(trimethylsilyl)ethoxy]methyl}-1H-1,5,7-triazainden-2-yl) aniline 5A (1 g, 2.35 mmol) in isopropyl acetate (9 mL) and water (1 mL) were added Na₂CO₃ (274 mg, 2.58 mmol) and phenyl chloroformate (0.35 mL, 2.58 mmol) sequentially at 0° C. The resulting reaction mixture was stirred at RT for 1 h. After completion of reaction (TLC monitoring), tert-butyl octahydro-1H-2,5-diazaindene-5-carboxylate 18 (532 mg, 2.35 mmol) and Et₃N(0.33 mL, 2.35 mmol) were added to the reaction at RT and stirred at 60° C. for 16 h. After completion of reaction (TLC and LCMS monitoring), the reaction mixture was poured into ice cold water (50 mL) and extracted with EtOAc (3×50 mL). The combined organic layer was washed with brine solution (2×50 mL), dried over Na₂SO₄, filtered and concentrated under reduced pressure. The crude was purified by flash chromatography using 30-40% EtOAc in heptane to get the desired product as a light brown solid 19 (1.2 g, 75%).

LCMS=[M+H]⁺: 678.49; Purity=82%.

### Synthesis of N-(4-(4-morpholino-7H-pyrrolo[2,3-d]pyrimidin-6-yl)phenyl)octahydro-2H-pyrrolo[3,4-c]pyridine-2-carboxamide (20)

To a stirred solution of tert-butyl 2-((4-(4-morpholino-7-((2-(trimethylsilyl)ethoxy)methyl)-7H-pyrrolo[2,3-d]pyrimidin-6-yl)phenyl)carbamoyl)octahydro-5H-pyrrolo[3,4-c]pyridine-5-carboxylate 19 (1.1 g, 1.62 mmol) in DCM (20 mL) was added TFA (10 mL) dropwise at 0° C. The reaction mixture was stirred at RT for 2 h. After completion of reaction (TLC monitoring), the reaction mixture was concentrated under reduced pressure. The crude was dissolved in 1,4-dioxane (10 mL) and 1,2-ethanediamine (0.33 mL, 4.87 mmol) was added at RT and stirred at 80° C. for 2 h. After completion of reaction (LCMS monitoring), the reaction mixture was diluted with 25% IPA in CHCl₃ (100 mL) and washed with ice cold water (3×50 mL). The combined organic layer was dried over Na₂SO₄, filtered and concentrated under reduced pressure to get the desired compound as an off white solid 20 (700 mg), crude was used in next step directly without purification. LCMS=[M+H]⁺: 448.23

### Synthesis of 5-acryloyl-N-(4-(4-morpholino-7H-pyrrolo[2,3-d]pyrimidin-6-yl)phenyl)octahydro-2H-pyrrolo[3,4-c]pyridine-2-carboxamide (Compound 606)

To a stirred solution of N-(4-(4-morpholino-7H-pyrrolo[2,3-d]pyrimidin-6-yl)phenyl)octahydro-2H-pyrrolo[3,4-c]pyridine-2-carboxamide 20 (700 mg, 1.56 mmol) in THF (10 mL) was added K₃PO₄ (664 mg, 3.13 mmol) dissolved in water (5 mL) at 0° C., followed by 3-chloropropionyl chloride 17 (0.18 mL, 1.88 mmol) dissolved in THF (2 mL) was added drop wise to the reaction at 0° C. The reaction mixture was stirred at RT for 1 h. After completion of SM (TLC and LCMS monitoring), 2N aq. NaOH (9 mL, 18.8 mmol) was added at 0° C. and stirred at 50° C. for 2 h. After completion of reaction (monitored by TLC and LCMS), the reaction mixture was poured into ice cold water (50 mL) and extracted with 2-MeTHF (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get Compound 606 (31 mg, 4%).

¹H NMR (400 MHz, DMSO-d₆): δ 12.07 (s, 1H), 8.25 (s, 1H), 8.15 (s, 1H), 7.77-7.75 (m, 2H), 7.59-7.57 (m, 2H), 7.04 (s, 1H), 6.83-6.77 (m, 1H), 6.15-6.09 (m, 1H), 5.71-5.66 (m, 1H), 3.95-3.92 (m, 1H), 3.87-3.85 (m, 4H), 3.75-3.73 (m, 4H), 3.69-3.61 (m, 1H), 3.55-3.45 (m, 3H), 3.16-3.08 (m, 1H), 2.44-2.38 (m, 4H), 1.73-1.66 (m, 1H), 1.40-1.34 (m, 1H).

LCMS=[M+H]⁺: 502.37; Purity=99.62%.

### Example 7

### Compound 609

### Preparation of N-(4-(4-(3-oxa-8-azabicyclo[3.2.1]octan-8-yl)-7H-pyrrolo[2,3-d]pyrimidin-6-yl)phenyl)-1′-acryloyl-[1,4′-bipiperidine]-4-carboxamide (Compound 609)

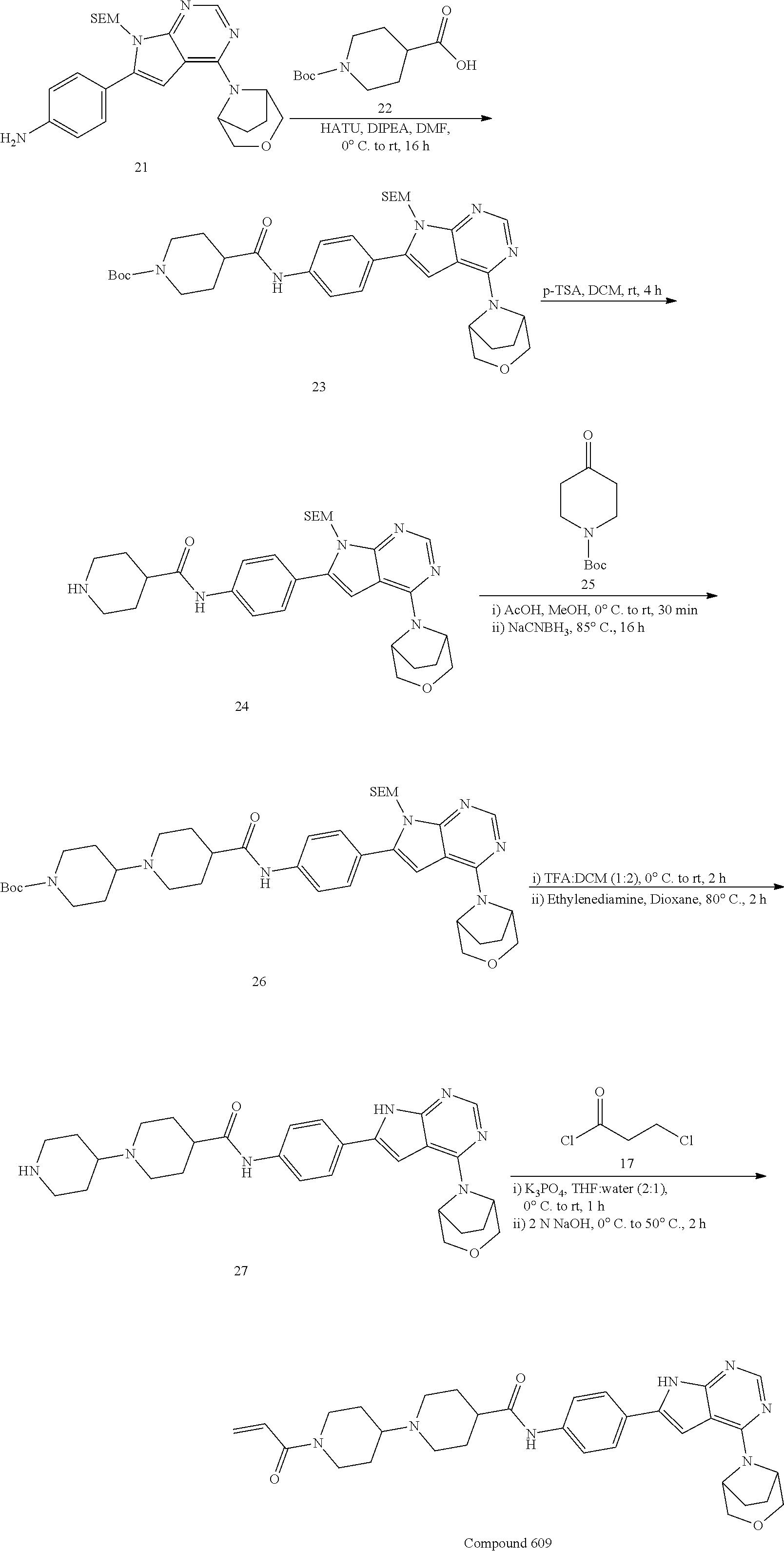

### Synthesis of tert-butyl 4-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)carbamoyl)piperidine-1-carboxylate (23)

To a stirred solution of 4-(4-(3-oxa-8-azabicyclo[3.2.1]octan-8-yl)-7-((2-(trimethylsilyl)ethoxy)methyl)-7H-pyrrolo[2,3-d]pyrimidin-6-yl) aniline 21 (1.5 g, 3.32 mmol) and1-tert-butoxycarbonyl-4-piperidinecarboxylic acid 22 (761 mg, 3.32 mmol) in DMF (25 mL) were added DIPEA (1.74 mL, 9.96 mmol) and HATU (1.89 g, 4.98 mmol) sequentially at 0° C. and the reaction mixture was stirred at RT for 16 h. After completion of reaction (monitoring by TLC), the reaction mass was poured into ice water (100 mL) and extracted with EtOAc (3×100 mL). The combined organic layer was dried over Na₂SO₄, filtered and concentrated under reduced pressure. The crude was purified by flash chromatography (silica gel 100-200 M, 40 g SNAP) using 30-40% EtOAc in heptane to get the title compound as a yellow solid 23 (1.5 g, 58%). LCMS=[M+H]⁺: 663.44; Purity=83%.

### Synthesis of N-(4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)piperidine-4-carboxamide (24)

To a stirred solution of tert-butyl 4-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)carbamoyl)piperidine-1-carboxylate 23 (1.5 g, 2.26 mmol) in DCM (20 mL) was added p-TsOH·H₂O (1.72 g, 9.06 mmol) at RT and stirred for 4 h. After completion of reaction (monitoring by TLC), the reaction mixture was quenched with sat. NaHCO₃ to pH~8-9 and extracted with 25% IPA in CHCl₃ (3×100 mL). The combined organic layer was dried over Na₂SO₄, filtered and concentrated under reduced pressure to get the title compound as a yellow solid 24 (1.2 g; crude), which was used in next step directly without further purification.

LCMS=[M+H]⁺: 563.36; Purity=67%.

### Synthesis of tert-butyl 4-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)carbamoyl)-[1,4′-bipiperidine]-1′-carboxylate (26)

To a stirred solution of N-(4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)piperidine-4-carboxamide 24 (1 g, 1.77 mmol) and tert-butyl 4-oxo-1-piperidinecarboxylate 25 (708 mg, 3.56 mmol) in MeOH (20 mL) was added acetic acid (0.2 mL, 3.56 mmol) at 0° C. under N₂ atmosphere and stirred for 30 min. After that, NaCNBH₃ (212 mg, 3.56 mmol) was added and stirred at 85° C. for 16 h. After completion of reaction (TLC and LCMS monitoring), the reaction mixture was quenched with sat. NaHCO₃ and extracted with 25% IPA in CHCl₃ (3×50 mL). The combined organic layer was dried over Na₂SO₄, filtered and concentrated under reduced pressure. The crude was purified by flash chromatography (silica gel 100-200 M, 12 g SNAP) using 5-6% MeOH in DCM to get the title compound as a yellow solid 26 (900 mg, 68%).

LCMS=[M+H]⁺: 746.49; Purity=91%.

### Synthesis of N-(4-(4-(3-oxa-8-azabicyclo[3.2.1]octan-8-yl)-7H-pyrrolo[2,3-d]pyrimidin-6-yl)phenyl)-[1,4′-bipiperidine]-4-carboxamide (27)

To a stirred solution of tert-butyl 4-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)carbamoyl)-[1,4′-bipiperidine]-1′-carboxylate 26 (650 mg, 0.87 mmol) in DCM (7 mL) was added TFA (3.5 mL) dropwise at 0° C. and the reaction mixture was stirred at RT for 2 h. After completion of reaction (TLC monitoring), the reaction mixture was concentrated under reduced pressure. The crude was dissolved in 1,4-dioxane (10 mL) and 1,2-ethanediamine (0.12 mL, 1.75 mmol) was added at RT and the reaction mixture was stirred at 80° C. for 2 h. After completion of reaction (LCMS monitoring), the reaction mixture was diluted with 25% IPA in CHCl₃ (100 mL) and washed with ice cold water (2×50 mL). The organic layer was dried over Na₂SO₄, filtered and concentrated under reduced pressure to get the title compound as an off white solid 27 (440 mg; crude), which was used in next LCMS=[M+H]⁺: 516.47; Purity=78%.

### Synthesis of N-(4-(4-(3-oxa-8-azabicyclo[3.2.1]octan-8-yl)-7H-pyrrolo[2,3-d]pyrimidin-6-yl)phenyl)-1′-acryloyl-[1,4′-bipiperidine]-4-carboxamide (Compound 609)

To a stirred solution of N-(4-(4-(3-oxa-8-azabicyclo[3.2.1]octan-8-yl)-7H-pyrrolo[2,3-d]pyrimidin-6-yl)phenyl)-[1,4′-bipiperidine]-4-carboxamide 27 (300 mg, 0.582 mmol) in THF (6 mL) was added K₃PO₄ (247 mg, 1.16 mmol) dissolved in water (3 mL) at 0° C., followed by 3-chloropropionyl chloride 17 (88.6 mg, 0.698 mmol) was added to this reaction mixture at 0° C. and the reaction mixture was stirred at RT for 1 h. After completion of SM (monitoring by TLC), 2N aq. NaOH (3.5 mL, 6.98 mmol) was added and the reaction mixture was stirred at 50° C. for 2 h. After completion of reaction (LCMS monitoring), the reaction mixture was quenched with ice cold water (50 mL) and extracted with 2-MeTHF (3×50 mL). The combined organic layer was dried over Na₂SO₄, filtered and concentrated under reduced pressure. The crude was purified by flash chromatography (silica gel 100-200 M, 12 g SNAP) using 5-10% MeOH in DCM to get Compound 609 (78 mg, 23%).

¹H NMR (400 MHz, DMSO-d₆): δ 12.11 (s, 1H), 9.92 (s, 1H), 8.15 (s, 1H), 7.83 (d, J=8.8 Hz, 2H), 7.65 (d, J=8.8 Hz, 2H), 7.04 (s, 1H), 6.83-6.76 (m, 1H), 6.07 (dd, J=17 Hz, 2 Hz, 1H), 5.64 (dd, J=10.4 Hz, 2 Hz, 1H), 4.90 (brs, 2H), 4.45-4.42 (m, 1H), 4.08-4.05 (m, 1H), 3.70-3.67 (m, 2H), 3.62-3.60 (m, 2H), 3.01 (t, J=12.8 Hz, 1H), 2.91-2.89 (m, 2H), 2.66-2.57 (m, 2H), 2.30-2.26 (m, 1H), 2.16 (t, J=10.8 Hz, 2H), 2.03-1.95 (m, 4H), 1.78-1.75 (m, 4H), 1.65-1.57 (m, 2H), 1.34-1.23 (m, 2H).

LCMS=[M+H]⁺: 570.56; Purity=98.96%.

### Example 8

### Compound 610

### Preparation of N-(4-(4-(3-oxa-8-azabicyclo[3.2.1]octan-8-yl)-7H-pyrrolo[2,3-d]pyrimidin-6-yl)phenyl)-1′-(but-2-ynoyl)-[1,4′-bipiperidine]-4-carboxamide (Compound 610)

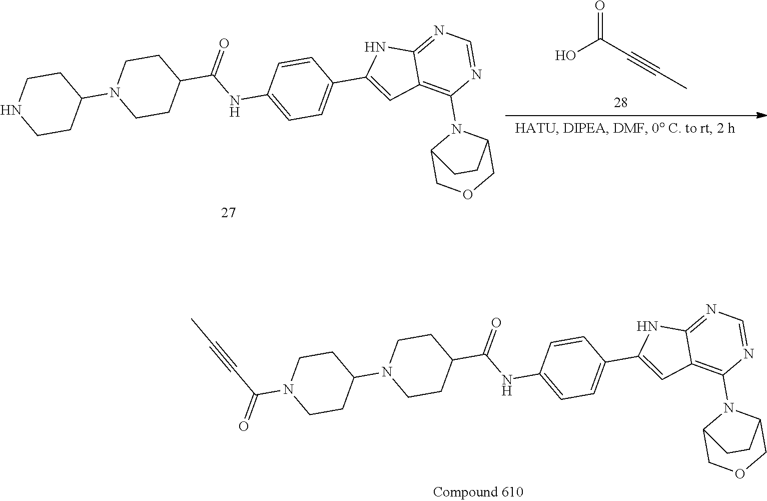

To a stirred solution of N-(p-{4-(3-oxa-8-azabicyclo[3.2.1]oct-8-yl)-1H-1,5,7-triazainden-2-yl}phenyl)-[1,4′-bipiperidyl]-4-carboxamide 27 (250 mg, 0.485 mmol) and 2-butynoic acid 28 (61.1 mg, 0.727 mmol) in DMF (5 mL) were added DIPEA (0.269 mL, 1.45 mmol) and HATU (369 mg, 0.970 mmol) sequentially at 0° C. and the reaction mixture was stirred at RT for 2 h. After completion of reaction (TLC monitoring), the reaction mixture was poured into ice cold water (50 mL) and extracted with 25% IPA in CHCl₃ (3×50 mL). The combined organic layer was dried over Na₂SO₄, filtered and concentrated under reduced pressure. The crude was purified by flash chromatography (silica gel 100-200 M, 12 g SNAP) using 5-10% MeOH in DCM to Compound 610 (120 mg, 42%).

¹H NMR (400 MHz, DMSO-d₆): δ 12.12 (s, 1H), 9.92 (s, 1H), 8.15 (s, 1H), 7.83 (d, J=8.4 Hz, 2H), 7.65 (d, J=8.8 Hz, 2H), 7.04 (s, 1H), 4.90 (brs, 2H), 4.32-4.25 (m, 2H), 3.70-3.68 (m, 2H), 3.63-3.60 (m, 2H), 3.09 (t, J=12 Hz, 1H), 2.91-2.88 (m, 2H), 2.67-2.61 (m, 2H), 2.33-2.29 (m, 1H), 2.18-2.15 (m, 2H), 2.03-1.95 (m, 7H), 1.79-1.75 (m, 4H), 1.67-1.58 (m, 2H), 1.38-1.30 (m, 1H), 1.27-1.21 (m, 1H).

LCMS=[M+H]⁺: 582.58; Purity=98.98%.

### Example 9

### Compound 607

### Preparation of (E)-N-(4-(4-(3-oxa-8-azabicyclo[3.2.1]octan-8-yl)-7H-pyrrolo[2,3-d]pyrimidin-6-yl)phenyl)-1′-(4-(dimethylamino)but-2-enoyl)-[1,4′-bipiperidine]-4-carboxamide (Compound 607)

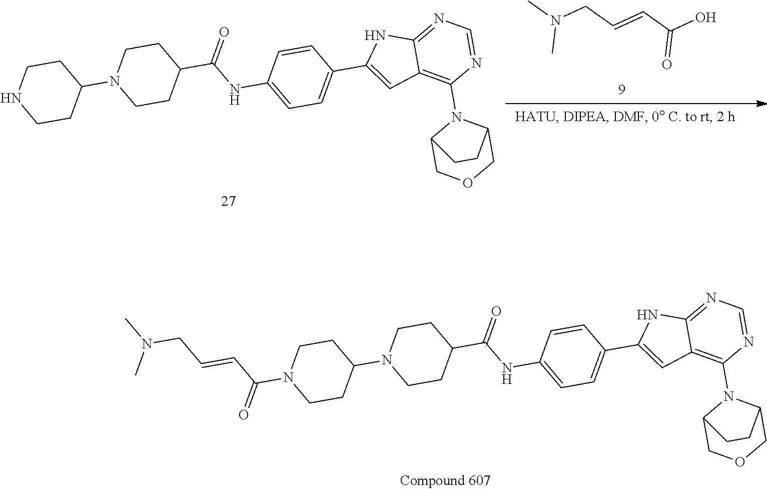

To a stirred solution of N-(p-{4-(3-oxa-8-azabicyclo[3.2.1]oct-8-yl)-1H-1,5,7-triazainden-2-yl}phenyl)-[1,4′-bipiperidyl]-4-carboxamide 27 (250 mg, 0.485 mmol) and (E)-4-(dimethylamino)but-2-enoic acid 9 (94 mg, 0.727 mmol) in DMF (10 mL) were added DIPEA (0.423 mL, 2.42 mmol) and HATU (369 mg, 0.970 mmol) sequentially at 0° C. under N₂ atmosphere. The reaction mixture was stirred at RT for 2 h. After completion of reaction (TLC monitoring), the reaction mixture was poured into ice cold water (50 mL) and extracted with 25% IPA in CHCl₃ (3×50 mL). The combined organic layer was dried over Na₂SO₄, filtered and concentrated under reduced pressure. The crude was purified by RP-HPLC [5-95% 0.1% TFA in Water/ACN; Column: Atlantis T3 (19*250) 10 μ] to get Compound 607 (87 mg, 28%).

¹H NMR (400 MHz, DMSO-d₆): δ 12.46 (s, 1H), 10.18 (s, 1H), 8.23 (s, 1H), 7.88 (d, J=8.8 Hz, 2H), 7.67 (d, J=8.4 Hz, 2H), 7.21-7.18 (m, 1H), 6.95-6.92 (m, 1H), 6.62-6.55 (m, 1H), 4.95 (brs, 2H), 4.59-4.56 (m, 1H), 4.19-4.06 (m, 1H), 3.88-3.85 (m, 2H), 3.73-3.64 (m, 4H), 3.13-2.97 (m, 3H), 2.77 (s, 6H), 2.62-2.58 (m, 4H), 2.11-2.02 (m, 9H), 1.96-1.866 (m, 2H), 1.59-1.51 (m, 2H).

LCMS=[M+H]⁺: 627.52; Purity=99.91%.

### Example 10

### Compound 608

### Preparation of (R)-4-((3-acrylamidopiperidin-1-yl)methyl)-N-(5-(4-morpholino-7H-pyrrolo[2,3-d]pyrimidin-6-yl)pyrimidin-2-yl)picolinamide (Compound 608)

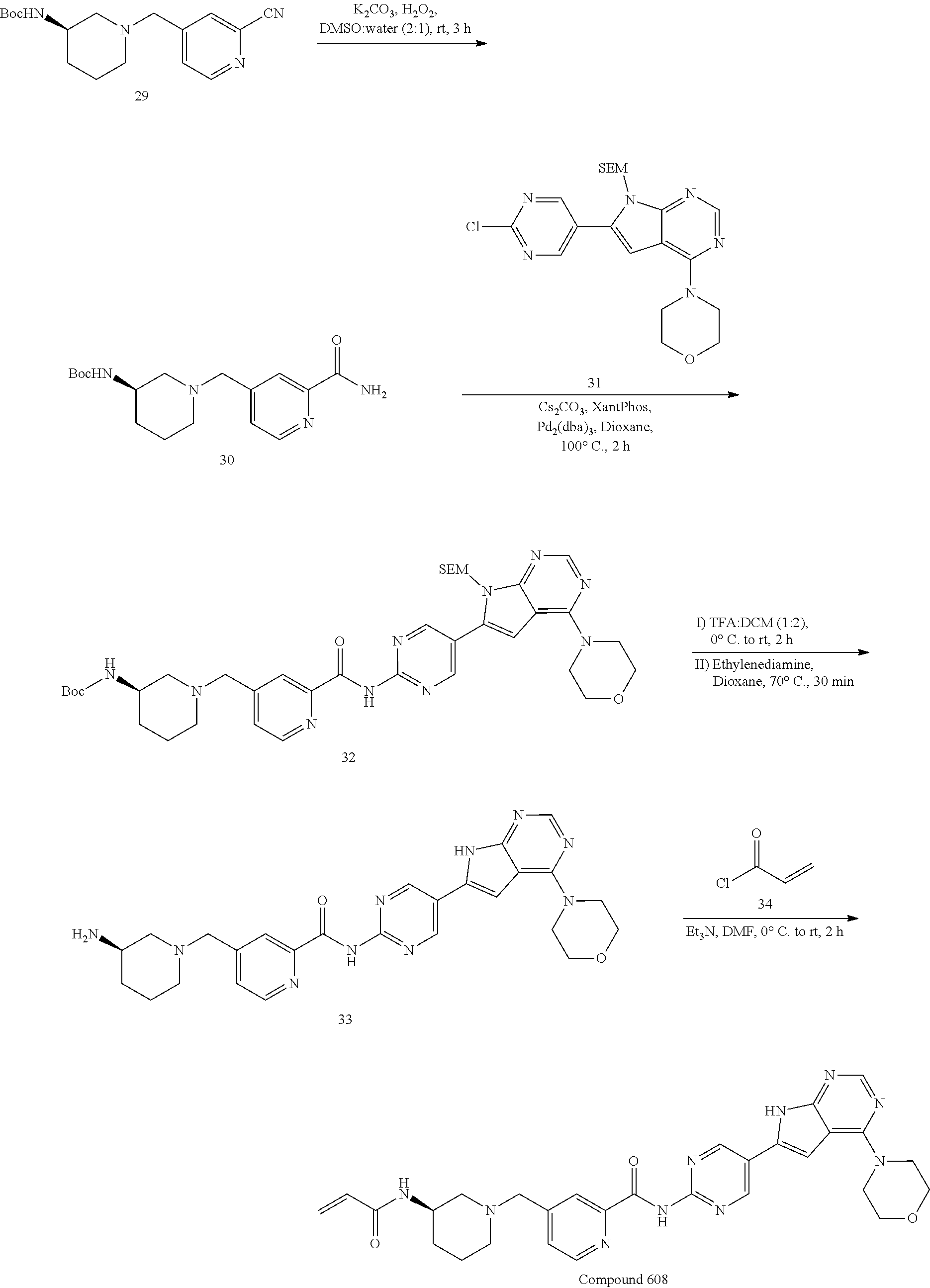

### Synthesis of tert-butyl(R)-(1-((2-carbamoylpyridin-4-yl)methyl)piperidin-3-yl)carbamate (30)

To a stirred solution of (R)-1-[(2-carbamoyl-4-pyridyl)methyl]-3-piperidylamino-tert-butylformylate 29 (1.2 g, 3.59 mmol) in DMSO (10 mL) and H₂O (5 mL) were added K₂CO₃ (874 mg, 6.32 mmol) and H₂O₂ (0.5 mL, 6.32 mmol; 30% w/w in H₂O) at RT under N₂ atmosphere. The reaction mixture was stirred at RT for 3 h. After completion of reaction (TLC monitoring), excess H₂O₂ was quenched with sat. Na₂S203 and extracted with EtOAc (2×100 mL). The combined organic layer was washed with brine (2×50 mL), dried over Na₂SO₄, filtered and concentrated under reduced pressure. The crude was purified by flash chromatography (silica gel 100-200 M, 12 g SNAP) using 40-50% EtOAc in heptane to get the desired product as a brown solid 30 (1.2 g, 49%).

LCMS=[M+H]⁺: 334.4; Purity=96.48%.

### Synthesis of tert-butyl(R)-(1-((2-((5-(4-morpholino-7-((2-(trimethylsilyl)ethoxy)methyl)-7H-pyrrolo[2,3-d]pyrimidin-6-yl)pyrimidin-2-yl)carbamoyl)pyridin-4-yl)methyl)piperidin-3-yl)carbamate (32)

To a stirred solution of tert-butyl(R)-(1-((2-carbamoylpyridin-4-yl)methyl)piperidin-3-yl)carbamate

30 (1.2 g, 3.59 mmol) and (2-{[2-(2-chloro-5-primidinyl)-4-morpholino-1H-1,5,7-triazainden-1-yl]methoxy}ethyl)tris(methyl)silane 31 (1.92 g, 4.31 mmol) in 1,4-dioxane (15 mL) was added Cs₂CO₃ (2.92 g, 8.97 mmol) at RT. The reaction mixture was degassed with N₂ gas for 20 min, followed by XantPhos (415 mg, 0.718 mmol) and Pd₂(dba)₃ (290 mg, 0.359 mmol) were added and the reaction mixture was stirred at 100° C. for 2 h. After completion of reaction (TLC and LCMS monitoring), the reaction mixture was diluted with water (100 mL) and extracted with EtOAc (2×100 mL). The combined organic layer was washed with brine solution (2×50 mL), dried over Na₂SO₄, filtered and concentrated under reduced pressure. The crude was purified by column chromatography using 60-70% EtOAc in heptane to get the title compound as a pale yellow solid 32 (1.3 g, 48%).

LCMS=[M+H]⁺: 745.46; Purity=76.45%.

### Synthesis of (R)-4-((3-aminopiperidin-1-yl)methyl)-N-(5-(4-morpholino-7H-pyrrolo[2,3-d]pyrimidin-6-yl)pyrimidin-2-yl)picolinamide (33)

To a stirred solution of tert-butyl(R)-(1-((2-((5-(4-morpholino-7-((2-(trimethylsilyl)ethoxy)methyl)-7H-pyrrolo[2,3-d]pyrimidin-6-yl)pyrimidin-2-yl)carbamoyl)pyridin-4-yl)methyl)piperidin-3-yl)carbamate 32 (1.3 g, 1.75 mmol) in DCM (12 mL) was added TFA (6 mL) drop wise at 0° C. and the reaction mixture was stirred at RT for 2 h. After completion of reaction (monitoring by TLC), the reaction mixture was concentrated under reduce pressure. The crude was dissolved in 1,4-dioxane (10 mL) and 1,2-ethanediamine (0.175 mL, 2.62 mmol) was added at RT and the reaction mixture was stirred at 70° C. for 30 min. After completion of reaction (monitoring by LCMS), the reaction mixture was diluted with 25% IPA in CHCl₃ (100 mL) and washed with ice cold water (2×50 mL). The combined organic layer was dried over Na₂SO₄, filtered and concentrated under reduced pressure to get the title compound as a yellow solid 33 (900 mg), crude was used in next step directly without further purification.

LCMS=[M+H]⁺: 515.31; Purity=63%.

### Synthesis of (R)-4-((3-acrylamidopiperidin-1-yl)methyl)-N-(5-(4-morpholino-7H-pyrrolo[2,3-d]pyrimidin-6-yl)pyrimidin-2-yl)picolinamide (Compound 608)

To a stirred solution of (R)-4-((3-aminopiperidin-1-yl)methyl)-N-(5-(4-morpholino-7H-pyrrolo[2,3-d]pyrimidin-6-yl)pyrimidin-2-yl)picolinamide 33 (300 mg, 0.583 mmol) in DMF (5 mL) was added Et₃N(0.244 mL, 1.75 mmol) at 0° C. and stirred for 10 min under N₂ atmosphere, followed by acryl chloride 34 (70.7 μL, 0.874 mmol) was added and the reaction mixture was stirred at RT for 1 h. After completion of reaction (TLC and LCMS monitoring), the reaction mixture was poured into ice cold water (50 mL) and extracted with 25% IPA in CHCl₃ (3×50 mL). The combined organic layer was washed with ice cold brine (2×50 mL), dried over Na₂SO₄, filtered and concentrated under reduce pressure. The crude was purified by RP-HPLC [5-95% 0.1% TFA in water/ACN; Column: Atlantis T3 (19*250) 10μ] to get Compound 608 (66 mg, 20%).

¹H NMR (400 MHz, DMSO-d₆): δ 12.60 (s, 1H), 9.27 (s, 2H), 8.87 (d, J=4.4 Hz, 1H), 8.41 (s, 1H), 8.25 (s, 1H), 7.85-7.83 (m, 1H), 8.35-8.32 (m, 1H), 7.47 (s, 1H), 6.23-6.01 (m, 2H), 5.66-5.63 (m, 1H), 4.04-4.02 (m, 1H), 3.92-3.91 (m, 4H), 3.78-3.66 (m, 8H), 3.38-3.30 (m, 2H), 2.95-2.93 (m, 1H), 1.90-1.86 (m, 2H), 1.73-1.63 (m, 1H), 1.45-1.39 (m, 1H).

LCMS=[M+H]⁺: 569.39; Purity=99.91%.

### Synthesis of 4-(6-(2-chloropyrimidin-5-yl)-7-((2-(trimethylsilyl)ethoxy)methyl)-7H-pyrrolo[2,3-d]pyrimidin-4-yl) morpholine (31)

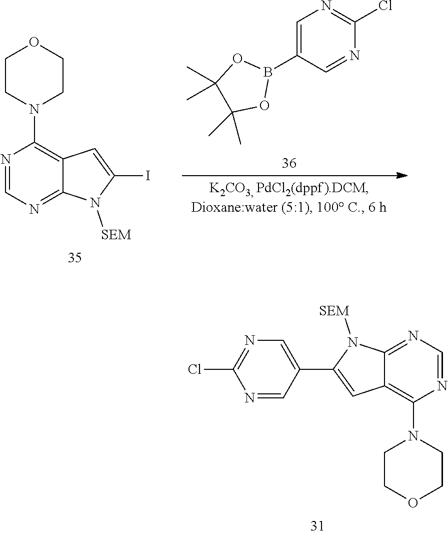 [SMILES: C[Si](C)(C)CCOCn1c(I)cc2c(N3CCOCC3)ncnc21 ; CC1(C)OB(c2cnc(Cl)nc2)OC1(C)C ; C[Si](C)(C)CCOCn1c(-c2cnc(Cl)nc2)cc2c(N3CCOCC3)ncnc21]

To a stirred solution of {2-[(2-iodo-4-morpholino-1H-1,5,7-triazainden-1-yl)methoxy]ethyl}tris(methyl) silane 35 (2.5 g, 5.43 mmol) and 2-(2-chloro-5-pyrimidinyl)-4,4,5,5-tetramethyl-1,3,2-dioxaborolane 36 (1.57 g, 6.52 mmol) in 1,4-dioxane (20 mL) and water (4 mL) was added K₂CO₃ (2.25 g, 16.29 mmol) at RT. The resulting reaction mixture was degassed with N₂ gas for 15 min, followed by PdCl₂(dppf).DCM (443 mg, 0.543 mmol) was added and the reaction mixture was stirred at 100° C. for 6 h. After the completion of reaction (TLC monitoring), the reaction mixture was filtered through Celite pad and washed with EtOAc. The filtrate was diluted with water (100 mL) and extracted with EtOAc (2×100 mL). The combined organic layer was washed with brine (2×100 mL), dried over Na₂SO₄, filtered and evaporated under reduced pressure. The crude was purified by flash chromatography using 40-50% EtOAc in heptane to get the desired product as a yellow solid 31 (2 g, 82%), LCMS=[M+H]⁺: 447.22; Purity=98.60%.

### Example 11

### Compound 701

Preparation of N,N-dimethyl-4-(4-(((4-(4-morpholino-7H-pyrrolo[2,3-d]pyrimidin-6-yl)phenyl)amino)methyl)piperidin-1-yl)but-2-ynamide (Compound 701)

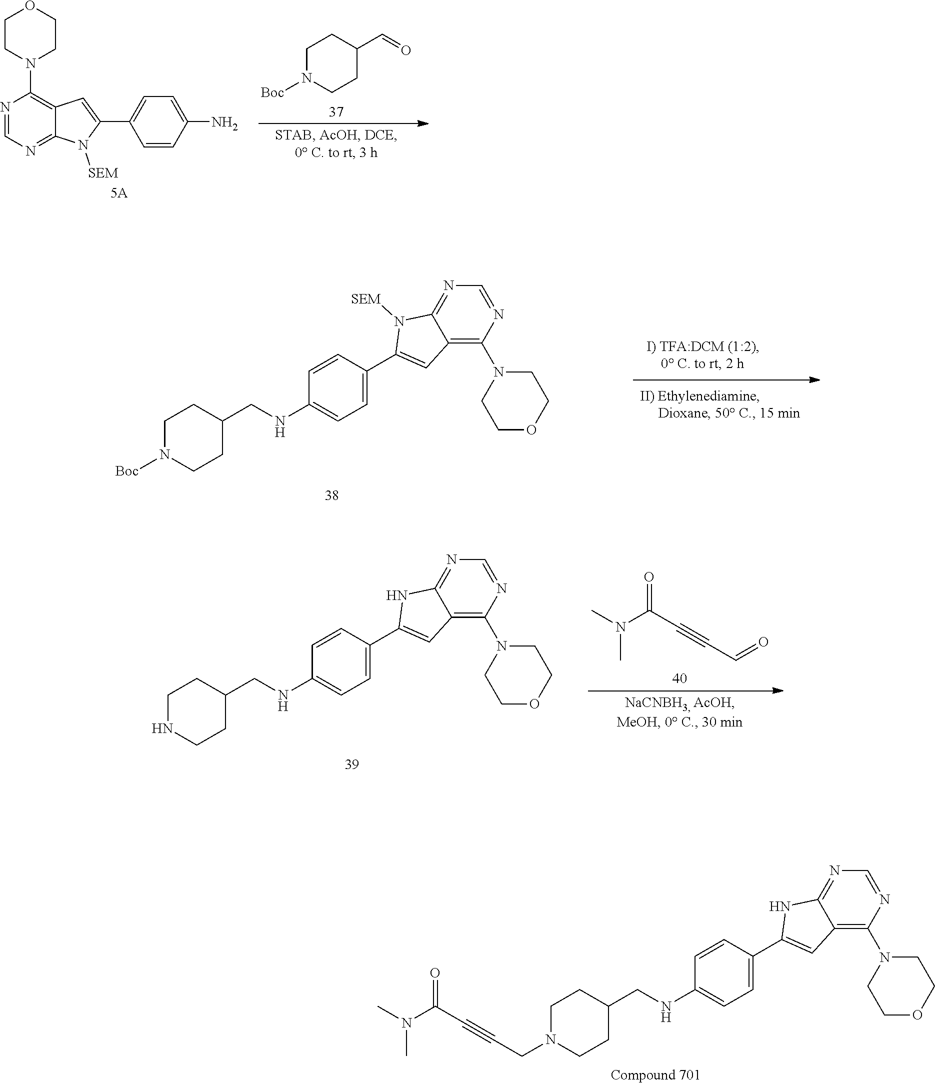

### Synthesis of tert-butyl 4-{[p-(4-morpholino-1-{[2-(trimethylsilyl)ethoxy]methyl}-1H-1,5,7-triazainden-2-yl)phenylamino]methyl}-1-piperidinecarboxylate (38)

To a stirred solution of 4-(4-morpholino-7-((2-(trimethylsilyl)ethoxy)methyl)-7H-pyrrolo[2,3-d]pyrimidin-6-yl) aniline 5A (2 g, 4.7 mmol) in DCE (20 mL) were added tert-butyl 4-formyl-1-piperidinecarboxylate 37 (1.8 g, 8.46 mmol) and AcOH (cat) sequentially at 0° C. and stirred for 30 min. Then, STAB (1.99 g, 9.4 mmol) was added to the reaction mixture at 0° C. The reaction mixture was stirred at RT for 3 h. After completion of reaction (TLC and LCMS monitoring), the reaction mass was quenched with sat. NaHCO₃ (50 mL) and extracted with DCM (3×50 mL). The combined organic layer was dried over Na₂SO₄, filtered and concentrated under reduced pressure. The crude was purified by flash column chromatography (silica gel 100-200 M, 12 g SNAP) using 10-15% EtOAc in heptane to get the desired compound as a light green solid 38 (1.1 g, 32%).

LCMS: [M+H]⁺: 623.58; Purity=85%.

### Synthesis of 4-(4-morpholino-7H-pyrrolo[2,3-d]pyrimidin-6-yl)-N-(piperidin-4-ylmethyl) aniline (39)

To a stirred solution of tert-butyl 4-{[p-(4-morpholino-1-{[2-(trimethylsilyl)ethoxy]methyl}-1H-1,5,7-triazainden-2-yl)phenylamino]methyl}-1-piperidinecarboxylate 38 (500 mg, 0.803 mmol) in DCM (6 mL) was added TFA (3 mL) dropwise at 0° C. and the reaction mixture was stirred at RT for 2 h. After completion of reaction (monitoring by TLC and LCMS), the reaction mixture was concentrated under reduce pressure. The crude was dissolved in 1,4-dioxane (8 mL) followed by 1,2-ethanediamine (53.6 μL, 0.803 mmol) was added and the reaction mixture was stirred at 70° C. for 1 h. After completion of reaction (monitoring by LCMS), the reaction mixture was diluted with 25% IPA in CHCl₃ (100 mL) and washed with brine (2×50 mL). The organic layer was dried over Na₂SO₄, and filtered and concentrated under reduced pressure to get the title compound as a light yellow solid 39 (345 mg; crude) which was used in next step directly without further purification. LCMS: [M+H]⁺: 393.32; Purity=67%.

### Synthesis of N,N-dimethyl-4-(4-{[p-(4-morpholino-1H-1,5,7-triazainden-2-yl)phenylamino]methyl}-1-piperidyl)-2-butynamide (Compound 701)

To a stirred solution of 4-(4-morpholino-7H-pyrrolo[2,3-d]pyrimidin-6-yl)-N-(piperidin-4-ylmethyl) aniline 39 (300 μm, 0.764 mmol) in MeOH (5 mL) were added N,N-dimethyl-3-formylpropiolamide 40 (191 mg, 1.53 mmol) and AcOH (43.7 μL, 0.764 mmol) respectively at 0° C. The reaction mixture was stirred at RT for 15 min. Then, the reaction mixture was again cooled to 0° C. and followed by NaBH₃CN(96.1 mg, 1.53 mmol) dissolved in MeOH (0.5 mL) was added and the reaction mixture was stirred at RT for 2 h. After completion of reaction (TLC and LCMS monitoring), the reaction mixture was quenched with sat. NaHCO₃ (10 mL) extracted with 25% IPA in CHCl₃ (3×50 mL). The combined organic layer was dried over Na₂SO₄, filtered and concentrated under reduced pressure. The crude was purified by flash chromatography (silica gel 100-200 M, 12 g SNAP) using 5-6.5% MeOH in DCM to get Compound 701 (45 mg, 11.5%).

¹H NMR (400 MHz, DMSO-d₆): δ 11.92 (s, 1H), 8.12 (s, 1H), 7.61 (d, J=8.4 Hz, 2H), 6.82 (d, J=2 Hz, 1H), 6.60 (d, J=8.8 Hz, 2H), 5.90 (t, J=5.2 Hz, 1H), 3.84-3.82 (m, 4H), 3.74-3.73 (m, 4H), 3.51 (brs, 2H), 3.14 (s, 3H), 2.94 (t, J=6 Hz, 2H), 2.84-2.81 (m, 5H), 2.14 (t, J=5.3 Hz, 2H), 1.78 (d, J=11.6 Hz, 2H), 1.52 (brs, 1H), 1.10-1.07 (m, 2H).

LCMS: [M+H]⁺: 501.6; Purity=98%.

### Example 12

### Compound 616

### Preparation of 1-(4-(2,2,2-trifluoro-1-((4-(4-morpholino-7H-pyrrolo[2,3-d]pyrimidin-6-yl)phenyl)amino)ethyl)-[1,4′-bipiperidin]-1′-yl)prop-2-en-1-one (Compound 616)

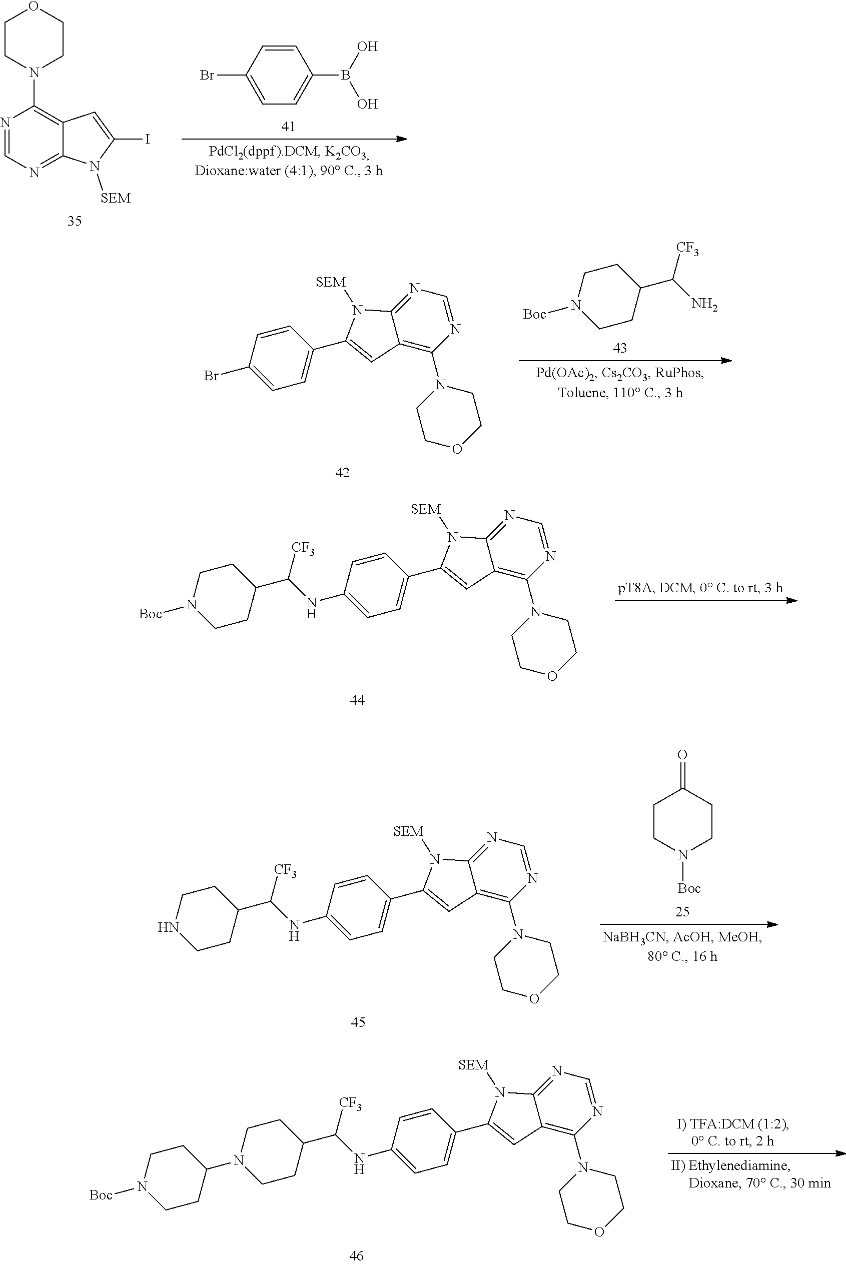

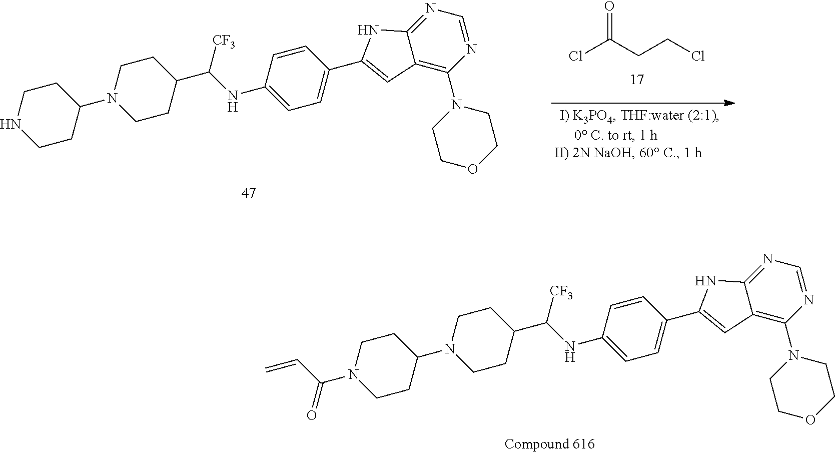 [SMILES: FC(F)(F)C(Nc1ccc(-c2cc3c(N4CCOCC4)ncnc3[nH]2)cc1)C1CCN(C2CCNCC2)CC1 ; O=C(Cl)CCCl ; C=CC(=O)N1CCC(N2CCC(C(Nc3ccc(-c4cc5c(N6CCOCC6)ncnc5[nH]4)cc3)C(F)(F)F)CC2)CC1]

### Synthesis of 4-(6-(4-bromophenyl)-7-((2-(trimethylsilyl)ethoxy)methyl)-7H-pyrrolo[2,3-d]pyrimidin-4-yl) morpholine (42)

To a stirred solution of {2-[(2-iodo-4-morpholino-1H-1,5,7-triazainden-1-yl)methoxy]ethyl}tris(methyl) silane 35 (5 g, 10.9 mmol) in 1,4-dioxane (40 mL) and water (10 mL) were added (p-bromophenyl)boranediol 41 (4.36 g, 21.7 mmol) and K₂CO₃ (4.5 g, 32.6 mmol) at RT. The resulting reaction mixture was degassed with argon gas for 15 min. Then, PdCl₂(dppf).DCM (886 mg, 1.09 mmol) was added and stirred at 90° C. for 3 h. After the completion of reaction (TLC monitoring), the reaction mixture was diluted with water (100 mL) and extracted with EtOAc (3×100 mL). The combined organic layer was washed with brine solution (2×50 mL), dried over Na₂SO₄, filtered and evaporated under reduced pressure. The crude was purified by flash column chromatography (silica gel 100-200 M, 40 g SNAP) using 30-35% EtOAc in heptane to get the title compound as a yellow solid 42 (4 g, 75%),

LCMS=[M+H]⁺: 489.26; Purity=88%.

### Synthesis of tert-butyl 4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl) piperidine-1-carboxylate (44)

To a solution of (2-{[2-(p-bromophenyl)-4-morpholino-1H-1,5,7-triazainden-1-stirred yl]methoxy}ethyl)tris(methyl)silane 42 (2 g, 4.09 mmol) and tert-butyl 4-(1-amino-2,2,2-trifluoroethyl)-1-piperidinecarboxylate 43 (1.61 g, 5.72 mmol) in toluene (30 mL) was added Cs₂CO₃ (3.99 g, 12.3 mmol). The resulting reaction mixture was degassed with N₂ gas for 10 min. Then, RuPhos (381 mg, 0.817 mmol) and Pd(OAc)₂ (91.7 mg, 0.409 mmol) was added to the reaction mixture and stirred at 110° C. for 3 h. After completion of reaction (TLC and LCMS monitoring), the reaction mixture was diluted with water (50 mL) and extracted with EtOAc (3×50 mL). The combined organic layer was washed with brine solution (2×50 mL), dried over anhydrous Na₂SO₄, filtered and concentrated under reduced pressure. The crude was purified by flash chromatography (silica gel 100-200 M, 24 g SNAP) using 30-40% EtOAc in heptane to get the title compound as a yellow solid 44 (2.5 g, 88%).

LCMS: [M+H]⁺: 691.56; Purity=71%.

### Synthesis of 4-(4-morpholino-7-((2-(trimethylsilyl)ethoxy)methyl)-7H-pyrrolo[2,3-d]pyrimidin-6-yl)-N-(2,2,2-trifluoro-1-(piperidin-4-yl)ethyl) aniline (45)

To an ice cold stirred solution of tert-butyl 4-{2,2,2-trifluoro-1-[p-(4-morpholino-1-{[2-(trimethylsilyl)ethoxy]methyl}-1H-1,5,7-triazainden-2-yl)phenylamino]ethyl}-1-piperidinecarboxylate 44 (1 g, 1.45 mmol) in DCM (20 mL) was added p-TSA·H₂O (1.1 g, 5.79 mmol) and the reaction mixture was stirred at RT for 3 h. After completion of the reaction (monitoring by TLC), the reaction mixture was quenched with saturated NaHCO₃ (50 mL) and extracted with DCM (2×100 mL). The organic layer was dried over Na₂SO₄, filtered and concentrated under reduced pressure to get the title compound as a yellow solid 45 (750 mg; crude) which was used in next step directly without further purification.

LCMS: [M+H]⁺: 591.44; Purity=71%.

### Synthesis of tert-butyl 4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)-[1,4′-bipiperidine]-1′-carboxylate (46)

To a stirred solution of [2,2,2-trifluoro-1-(4-piperidyl)ethyl][p-(4-morpholino-1-{[2-(trimethylsilyl)ethoxy]methyl}-1H-1,5,7-triazainden-2-yl)phenyl]amine 45 (700 mg, 1.18 mmol) and tert-butyl 4-oxo-1-piperidinecarboxylate 25 (590 mg, 2.96 mmol) in MeOH (15 mL) was added AcOH (0.136 mL, 2.37 mmol) at 0° C. under N₂ atmosphere and stirred for 30 min. After that, NaCNBH₃ (142 mg, 2.37 mmol) was added and stirred at 80° C. for 16 h. After completion of reaction (TLC and LCMS monitoring), the reaction mixture was quenched with saturated NaHCO₃ and extracted with 25% IPA in CHCl₃ (3×50 mL). The combined organic layer was dried over Na₂SO₄, filtered and concentrated under reduced pressure. The crude was purified by flash chromatography (silica gel 100-200 M, 12 g SNAP) using 8-10% MeOH in DCM to get the title compound as a yellow sticky solid 46 (900 mg, 78%).

LCMS: [M+H]⁺: 774.63; Purity=83%.

### Synthesis of N-(1-([1,4′-bipiperidin]-4-yl)-2,2,2-trifluoroethyl)-4-(4-morpholino-7H-pyrrolo[2,3-d]pyrimidin-6-yl) aniline (47)

To a stirred solution of tert-butyl 4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)-[1,4′-bipiperidine]-1′-carboxylate 46 (1 g, 1.29 mmol) in DCM (10 mL) was added TFA (5 mL) dropwise at 0° C. and the reaction mixture was stirred at RT for 2 h. After completion of reaction (TLC monitoring), the reaction mixture was concentrated under reduced pressure. The crude was dissolved in 1,4-dioxane (10 mL), followed by 1,2-ethanediamine (0.18 mL, 2.58 mmol) was added and stirred at 70° C. for 30 min. After completion of reaction (LCMS monitoring), the reaction mixture was poured into ice cold water (50 mL) and extracted with 25% IPA in CHCl₃ (3×50 mL). The combined organic layer was dried over Na₂SO₄, filtered and concentrated under reduced pressure to get the title compound as a green solid 47 (700 mg; crude) which was used in next step directly without further purification.

LCMS: [M+H]⁺: 544.50

### Synthesis of 1-(4-(2,2,2-trifluoro-1-((4-(4-morpholino-7H-pyrrolo[2,3-d]pyrimidin-6-yl)phenyl)amino)ethyl)-[1,4′-bipiperidin]-1′-yl)prop-2-en-1-one (Compound 616)

To a stirred solution of N-(1-([1,4′-bipiperidin]-4-yl)-2,2,2-trifluoroethyl)-4-(4-morpholino-7H-pyrrolo[2,3-d]pyrimidin-6-yl) aniline 47 (500 mg, 0.920 mmol) in THF (6 mL) was added K₃PO₄ (390 mg, 1.84 mmol) dissolved in water (3 mL) at 0° C., followed by 3-chloropropionyl chloride 17 (140 mg, 1.1 mmol) was added and the reaction mixture was stirred at RT for 1 h. Then, 2N aq. NaOH (5.5 mL, 11 mmol) was added dropwise at 0° C. and stirred at 60° C. for 1 h. After completion of reaction (LCMS monitoring), the reaction mixture was poured into ice cold water (50 mL) and extracted with 2-MeTHF (3×50 mL). The combined organic layer was dried over Na₂SO₄, filtered and concentrated under reduced pressure. The crude was purified by flash chromatography (silica gel 100-200 M, 12 g SNAP) using 8-10% MeOH in DCM to get Compound 616 (40 mg, 8%).

¹H NMR (400 MHz, DMSO-d₆): δ 11.95 (s, 1H), 8.13 (s, 1H), 7.63 (d, J=8.4 Hz, 2H), 6.87-6.81 (m, 3H), 6.79-6.75 (m, 1H), 6.06 (d, J=14.4 Hz, 2H), 5.63 (d, J=12 Hz, 1H), 4.43-4.40 (m, 1H), 4.30-4.27 (m, 1H), 4.05-4.00 (m, 1H), 3.85-3.83 (m, 4H), 3.74-3.73 (m, 4H), 3.02-2.96 (m, 1H), 2.89-2.84 (m, 2H), 2.61-2.57 (m, 2H), 2.19-2.17 (m, 2H), 1.90-1.62 (m, 5H), 1.53-1.39 (m, 2H), 1.33-1.23 (m, 2H).

LCMS=[M+H]⁺: 598.47; Purity=95.36%.

### Example 13

### Compound 617

### Preparation of N-(4-(4-(3-oxa-8-azabicyclo[3.2.1]octan-8-yl)-7H-pyrrolo[2,3-d]pyrimidin-6-yl)phenyl)-5-(((R)-3-acrylamidopiperidin-1-yl)methyl)-2-fluorobenzamide (Compound 617)

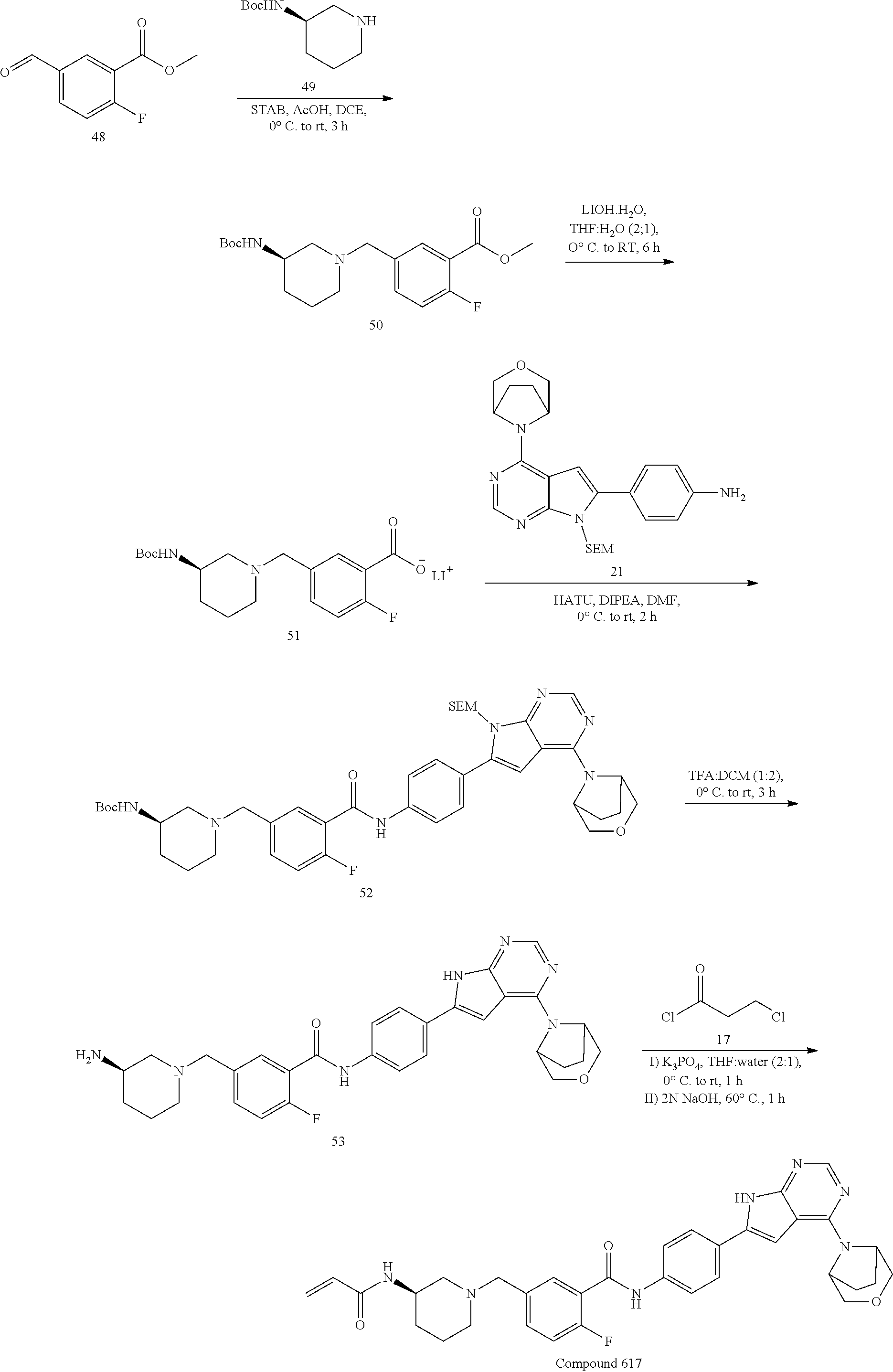

### Synthesis of methyl (R)-5-((3-((tert-butoxycarbonyl)amino)piperidin-1-yl)methyl)-2-fluorobenzoate (50)

To a stirred solution of methyl 2-fluoro-5-formylbenzoate 48 (800 mg, 4.39 mmol) and tert-butyl(R)-3-piperidylcarbamate 49 (880 mg, 4.39 mmol) in DCM (15 mL) was added acetic acid (0.5 mL, 8.78 mmol) at 0° C. and stirred for 20 min. Then, STAB (1.86 g, 8.78 mmol) was added and stirred at RT for 3 h. After completion of reaction (TLC and LCMS monitoring), the reaction mixture was quenched with saturated NaHCO₃ and extracted with 25% IPA in CHCl₃ (3×50 mL). The combined organic layer was dried over Na₂SO₄, filtered and concentrated under reduced pressure. The crude was purified by flash column chromatography (silica gel 100-200 M, 12 g SNAP) using 40-50% EtOAc in heptane to get the desired compound as a pale yellow solid 50 (1 g, 62%)

LCMS: [M+H]⁺: 367.28; Purity=82%.

### Synthesis of (R)-5-((3-((tert-butoxycarbonyl)amino)piperidin-1-yl)methyl)-2-fluorobenzoate (51)

To a stirred solution of methyl 5-({(R)-3-[(tert-butyl) (oxycarbonylamino)]-1-piperidyl}methyl)-2-fluorobenzoate 50 (1 g, 2.73 mmol) in THF (10 mL) was added LiOH·H₂O (573 mg, 13.6 mmol) dissolved with H₂O (5 mL) dropwise at 0° C. The resulting reaction mixture was stirred at RT for 6 h. After completion of reaction (TLC monitoring), the reaction mixture was concentrated under reduced pressure. The crude was dissolved in 25% IPA in CHCl₃ (50 mL) and stirred for few minute, then filtered through sintered funnel. The filtrate was concentrated under reduced pressure to get the desired compound as a pale yellow solid 51 (900 mg; as Li⁺ salt), crude was used in the next step directly without any purification.

LCMS: [M+H]⁺353.31; Purity=89%.

### Synthesis of tert-butyl((3R)-1-(3-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)carbamoyl)-4-fluorobenzyl)piperidin-3-yl)carbamate (52)

To an ice cold stirred solution of lithium 5-({(R)-3-[(tert-butyl) (oxycarbonylamino)]-1-piperidyl}methyl)-2-fluorobenzoate 51 (500 mg, 1.4 mmol) and p-[4-(3-oxa-8-azabicyclo[3.2.1]oct-8-yl)-1-{[2-(trimethylsilyl)ethoxy]methyl}-1H-1,5,7-triazainden-2-yl]aniline 21 (630 mg, 1.4 mmol) in DMF (10 mL) were added DIPEA (0.721 mL, 4.19 mmol) and HATU (1.06 g, 2.79 mmol) respectively under N₂ atmosphere. The resulting reaction mixture was stirred at RT for 2 h. After completion of reaction (TLC monitoring), the reaction mixture was poured into ice cold water (50 mL) and extracted with EtOAc (3×50 mL). The combined organic layer was dried over Na₂SO₄, filtered and concentrated under reduced pressure. The crude was purified by flash chromatography (silica gel 100-200 M, 12 g SNAP) using 3-5% MeOH in DCM to get the title compound as a pale yellow solid 52 (500 mg, 54%)

LCMS=[M+H]′: 786.56; Purity=70%.

### Synthesis of N-(4-(4-(3-oxa-8-azabicyclo[3.2.1]octan-8-yl)-7H-pyrrolo[2,3-d]pyrimidin-6-yl)phenyl)-5-(((R)-3-aminopiperidin-1-yl)methyl)-2-fluorobenzamide (53)

To a stirred solution of (R)-1-[(3-{N-p-[4-(3-oxa-8-azabicyclo[3.2.1]oct-8-yl)-1-{[2-(trimethylsilyl)ethoxy]methyl}-1H-1,5,7-triazainden-2-yl]phenylcarbamoyl}-4-fluorophenyl)methyl]-3-piperidyl 2-methyl-2-propanecarbamate 52 (400 mg, 0.509 mmol) in DCM (4 mL) was added TFA (2 mL) dropwise at 0° C. The reaction mixture stirred at RT for 2 h. After completion of reaction (TLC monitoring), the reaction mixture was concentrated under reduced pressure. The crude was dissolved in 1,4-dioxane (4 mL), followed by 1,2-ethanediamine (0.1 mL, 1.53 mmol) was added to this reaction mixture at RT and stirred at 70° C. for 1 h. After completion of reaction (LCMS monitoring), the reaction mixture was diluted with 25% IPA in CHCl₃ (50 mL) and washed with water (2×50 mL). The organic layer was dried over Na₂SO₄, filtered and concentrated under reduced pressure to get the desired compound as a pale yellow solid 53 (250 mg; crude) which was used in next step directly without further purification.

LCMS=[M−H]: 554.47; Purity=65%.

### Synthesis of N-(4-(4-(3-oxa-8-azabicyclo[3.2.1]octan-8-yl)-7H-pyrrolo[2,3-d]pyrimidin-6-yl)phenyl)-5-(((R)-3-acrylamidopiperidin-1-yl)methyl)-2-fluorobenzamide (Compound 617)

To a stirred solution of N-(p-{4-(3-oxa-8-azabicyclo[3.2.1]oct-8-yl)-1H-1,5,7-triazainden-2-yl}phenyl) 5-{[(R)-3-amino-1-piperidyl]methyl}-2-fluorobenzamide 53 (230 mg, 0.414 mmol) in THF (5 mL) was added K₃PO₄ (351 mg, 1.66 mmol) was dissolved in water (2.5 mL) at 0° C. Then, 3-chloropropionyl chloride 17 (78.8 mg, 0.621 mmol) was added to the reaction mixture and stirred at RT for 1 h. After completion of reaction (TLC monitoring), 2N aq. NaOH (2.5 mL, 4.97 mmol) was added dropwise at 0° C. and the resulting reaction mixture was stirred at 60° C. for 2 h. After completion of reaction (LCMS monitoring), the reaction mixture was poured into ice cold water (50 mL) and extracted with 2-MeTHF (3×50 mL). The combined organic layer was dried over Na₂SO₄, filtered and concentrated under reduced pressure. The crude was purified by RP-HPLC [5-95% 5 mM Ammonium acetate in water/ACN; Column: X select phenyl Hexyl column) to get Compound 617 (44 mg, 17%).

¹H NMR (400 MHz, DMSO-d₆): δ 12.18 (s, 1H), 10.48 (s, 1H), 8.17 (s, 1H), 7.96 (d, J=8.4 Hz, 1H), 7.91 (d, J=8.8 Hz, 2H), 7.77 (d, J=8.8 Hz, 2H), 7.57-7.56 (m, 1H), 7.52-7.49 (m, 1H), 7.30 (t, J=8.4 Hz, 1H), 7.10 (brs, 1H), 6.25-6.18 (m, 1H), 6.04 (dd, J=17.2 Hz, 2 Hz, 1H), 5.54 (dd, J=10 Hz, 2.4 Hz, 1H), 4.91 (brs, 2H), 3.80-3.78 (m, 1H), 3.71-3.69 (m, 2H), 3.63-3.61 (m, 2H), 3.56-3.47 (m, 2H), 2.76-2.74 (m, 1H), 2.62-2.61 (m, 1H), 2.04-1.99 (m, 5H), 1.87-1.82 (m, 1H), 1.75-1.66 (m, 2H), 1.51-1.45 (m, 1H), 1.23-1.18 (m, 1H). LCMS=[M+H]⁺: 610.46; Purity=96.34%.

### Example 14

### Compound 618

### Preparation of (R)-4-((3-(4-(3,3-difluoroazetidin-1-yl)but-2-ynamido)piperidin-1-yl)methyl)-N-(4-(4-morpholino-7H-pyrrolo[2,3-d]pyrimidin-6-yl)phenyl)picolinamide (Compound 618)

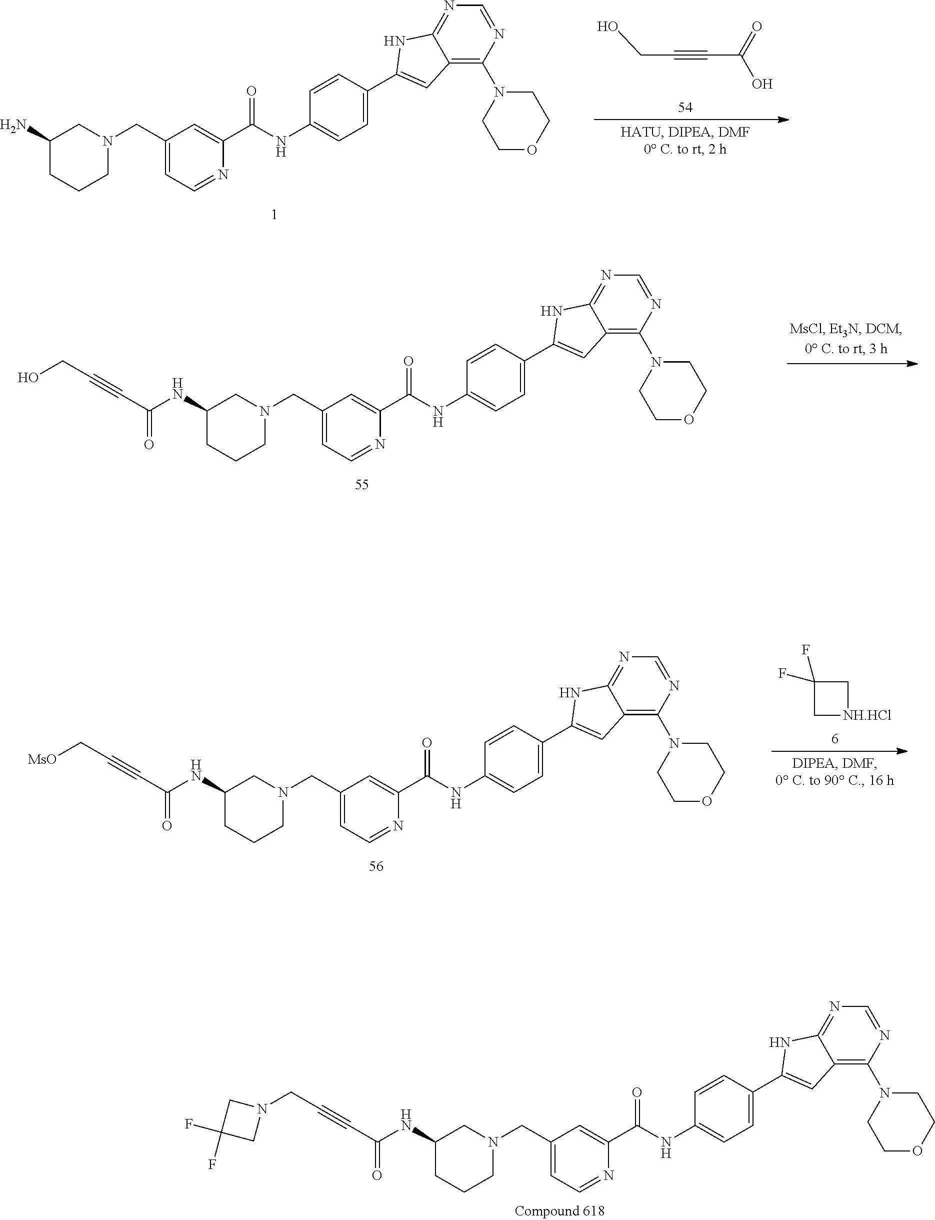

### Synthesis of (R)-4-((3-(4-hydroxybut-2-ynamido)piperidin-1-yl)methyl)-N-(4-(4-morpholino-7H-pyrrolo[2,3-d]pyrimidin-6-yl)phenyl)picolinamide (55)

To a stirred solution of N-[p-(4-morpholino-1H-1,5,7-triazainden-2-yl)phenyl]-4-{[(R)-3-amino-1-piperidyl]methyl}-2-pyridinecarboxamide 1 (400 mg, 0.780 mmol) and 4-hydroxy-2-butynoic acid 54 (93.7 mg, 0.936 mmol) in DMF (5 mL) were added DIPEA (0.4 mL, 2.34 mmol) and HATU (445 mg, 1.17 mmol) sequentially at 0° C. The resulting reaction mixture was stirred at RT for 2 h. After completion of reaction (TLC and LCMS monitoring), the reaction mixture was poured into ice-cold water (50 mL) and extracted with EtOAc (3×50 mL). The combined organic layer was washed with ice cold brine (2×50 mL), dried over Na₂SO₄, filtered and concentrated under reduced pressure to get the title compound as a light yellow solid 55 (400 mg; crude) which was used in the next step without purification.

LCMS=[M+H]⁺: 595.35; Purity=77%.

### Synthesis of (R)-4-((1-((2-((4-(4-morpholino-7H-pyrrolo[2,3-d]pyrimidin-6-yl)phenyl)carbamoyl)pyridin-4-yl)methyl)piperidin-3-yl)amino)-4-oxobut-2-yn-1-yl methanesulfonate (56)

To a stirred solution of (R)-4-((3-(4-hydroxybut-2-ynamido)piperidin-1-yl)methyl)-N-(4-(4-morpholino-7H-pyrrolo[2,3-d]pyrimidin-6-yl)phenyl)picolinamide 55 (400 mg, 0.673 mmol) in DCM (10 mL) were added Et₃N(0.23 mL, 1.68 mmol) and MsCl (0.078.1 mL, 1.01 mmol) at 0° C. under N₂ atmosphere. The resulting reaction mixture was stirred at RT for 3 h. After completion of reaction (TLC monitoring), the reaction mixture was quenched with NaHCO₃ (50 mL) and extracted with DCM (3×50 mL). The combined organic layer was dried over Na₂SO₄, filtered and concentrated under reduced pressure to get the desired compound as a yellow solid 56 (400 mg; crude) which was used in next step without any purification.

LCMS=[M+H]⁺: 673.46

### Synthesis of (R)-4-((3-(4-(3,3-difluoroazetidin-1-yl)but-2-ynamido)piperidin-1-yl)methyl)-N-(4-(4-morpholino-7H-pyrrolo[2,3-d]pyrimidin-6-yl)phenyl)picolinamide (Compound 618)

To a stirred solution of (R)-4-((1-((2-((4-(4-morpholino-7H-pyrrolo[2,3-d]pyrimidin-6-yl)phenyl)carbamoyl)pyridin-4-yl)methyl)piperidin-3-yl)amino)-4-oxobut-2-yn-1-yl methanesulfonate 56 (400 mg, 0.595 mmol) DMF (5 mL) were added 3,3-difluoroazetidine hydrogen chloride 6 (92.4 mg, 0.713 mmol) abd DIPEA (0.31 mL, 1.78 mmol) at 0° C. The resulting reaction mixture was stirred at 90° C. for 16 h. After completion of reaction (TLC monitoring), the reaction mass was poured into ice cold water (50 mL) and extracted with EtOAc (3×50 mL). The combined organic layer was washed with ice cold brine (2×50 mL), dried over Na₂SO₄, filtered and evaporated under reduced pressure. The crude was purified by RP-HPLC [5-95% 5 mM ABC in Water/ACN; Column: X BRIDGE C8 (19*250) 10μ] to get Compound 618 (7 mg, 1.67%). ¹H NMR (400 MHz, DMSO-d₆): δ 12.20 (s, 1H), 10.74 (s, 1H), 8.89 (d, J=4.8 Hz, 1H), 8.57 (d, J=7.6 Hz, 1H), 8.18 (s, 1H), 8.10 (s, 1H), 7.99 (d, J=8.8 Hz, 2H), 7.91 (d, J=8.8 Hz, 2H), 7.61 (d, J=4.0 Hz, 1H), 7.16 (s, 1H), 3.90-3.87 (m, 4H), 3.80 (m, 1H), 3.76-3.74 (m, 4H), 3.68-3.62 (m, 6H), 3.57 (s, 2H), 2.78-2.77 (m, 1H), 2.65-2.63 (m, 1H), 2.00-1.95 (m, 1H), 1.92-1.87 (m, 1H), 1.76-1.72 (m, 2H), 1.55-1.45 (m, 1H), 1.26-1.17 (m, 1H).

LCMS=[M+H]⁺: 670.42; Purity=95.37%.

### Example 15

### Compound 619

### Preparation of 1-(4-(((4-(4-morpholino-7H-pyrrolo[2,3-d]pyrimidin-6-yl)phenyl)amino)methyl)-[1,4′-bipiperidin]-1′-yl)prop-2-en-1-one (Compound 619)

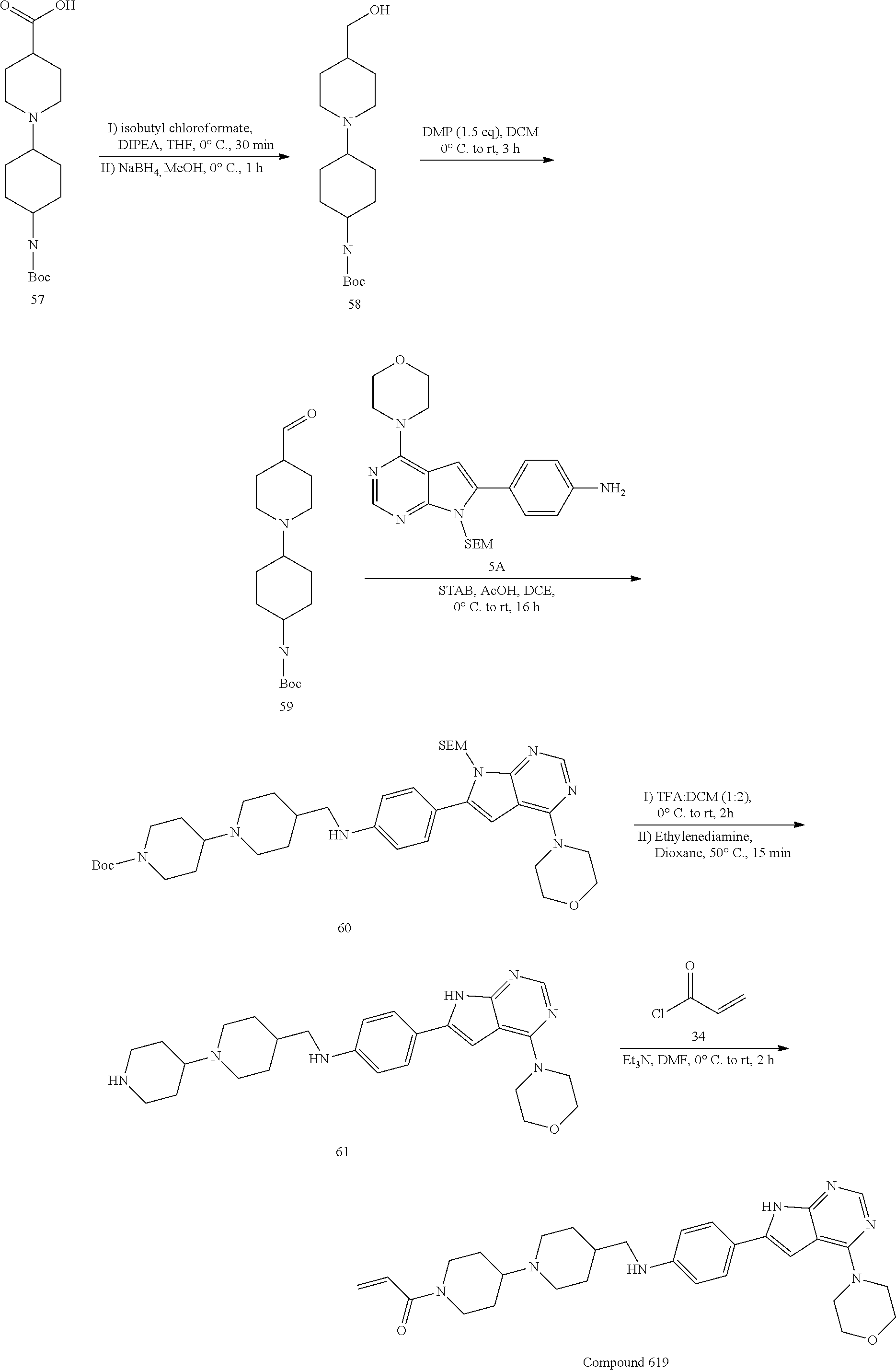

### Synthesis of tert-butyl 4-(hydroxyl methyl)-[1,4′-bipiperidine]-1′-carboxylate (58)

To a stirred solution of l′-tert-butoxycarbonyl-[1,4′-bipiperidyl]-4-carboxylic acid 57 (1 g, 3.2 mmol) in THF (16 mL) was added DIPEA (1.12 mL, 6.4 mmol) at 0° C. and stirred for 10 min. Then, isobutyl chloroformate (0.41 mL, 3.2 mmol) was added and stirred for 30 min at 0° C. After that, NaBH₄ (484 mg, 12.8 mmol) was dissolved in MeOH (3 mL) added dropwise to the reaction mixture at 0° C. and stirred at RT for 1 h. After completion of reaction (TLC and LCMS monitoring), the reaction mixture was quenched with sat. NH₄Cl and extracted with DCM (3×50 mL). The combined organic layer was washed with brine solution (2×50 mL), dried over Na₂SO₄, filtered and concentrated under reduced pressure to get the title compound as yellow liquid 58 (800 mg; crude) which was used in next step without any purification.

### Synthesis of tert-butyl 4-formyl-[1,4′-bipiperidine]-1′-carboxylate (59)

To a stirred solution of tert-butyl 4-(hydroxy methyl)-[1,4′-bipiperidyl]-1′-carboxylate 58 (800 mg, 2.68 mmol) in DCM (15 mL) was added DMP (1.71 g, 4.02 mmol) portion wise at 0° C. The reaction mixture was stirred at RT for 2 h. After completion reaction (monitoring by TLC), the reaction mixture was quenched with sat. NaHCO₃ and extracted with DCM (3×50 mL). The combined organic layer was washed with brine (2×50 mL), dried over Na₂SO₄, filtered and concentrated under reduced pressure to get the title compound as brown liquid 59 (700 mg; crude) which was forwarded to next step without any purification.

### Synthesis of tert-butyl 4-(((4-(4-morpholino-7-((2-(trimethylsilyl)ethoxy)methyl)-7H-pyrrolo[2,3-d]pyrimidin-6-yl)phenyl)amino)methyl)-[1,4′-bipiperidine]-1′-carboxylate (60)

To a stirred solution of p-(4-morpholino-1-{[2-(trimethylsilyl)ethoxy]methyl}-1H-1,5,7-triazainden-2-yl) aniline 5A (600 mg, 1.41 mmol) and tert-butyl 4-formyl-[1,4′-bipiperidyl]-1′-carboxylate 59 (501 mg, 1.69 mmol) in DCE (10 mL) was added acetic acid (0.161 mL, 2.82 mmol) at 0° C. and stirred at RT for 30 min. Then, STAB (896 mg, 4.23 mmol) was added at 0° C. and the reaction mixture was stirred at RT for 16 h. After completion of reaction (TLC and LCMS monitoring), the reaction mixture was quenched with sat. NaHCO₃ (50 mL) and extracted with 25% IPA in CHCl₃ (3×50 mL). The combined organic layer was dried over Na₂SO₄, filtered and concentrated under reduced pressure. The crude was purified by flash column chromatography (silica gel 100-200 M, 12 g SNAP) using 5-10% MeOH in DCM to obtain desired compound as a brown solid 60 (400 mg).

LCMS=[M+H]⁺: 706.68; Purity=64%.

### Synthesis of N-([1,4′-bipiperidin]-4-ylmethyl)-4-(4-morpholino-7H-pyrrolo[2,3-d]pyrimidin-6-yl) aniline (61)

To a stirred solution of tert-butyl 4-{[p-(4-morpholino-1-{[2-(trimethylsilyl)ethoxy]methyl}-1H-1,5,7-triazainden-2-yl)phenylamino]methyl}-[1,4′-bipiperidyl]-1′-carboxylate 60 (320 mg, 0.453 mmol) in DCM (4 mL) was added TFA (2 mL) dropwise at 0° C. The resulting reaction mixture was stirred at RT for 2 h. After completion of reaction (LCMS monitoring), the reaction mixture was concentrated under reduced pressure. The crude was dissolved in 1,4-dioxane (3 mL) followed by, 1,2-ethanediamine (90.8 μL, 1.36 mmol) was added to the reaction mixture at RT and stirred at 70° C. for 1 h. After completion of reaction (LCMS monitoring), the reaction mixture was diluted with water (50 mL) and extracted with 25% IPA in CHCl₃ (3×50 mL). The combined organic layer was dried over Na₂SO₄, filtered and concentrated under reduced pressure to get the title compound as a pale yellow solid 61 (200 mg; crude) which was used in next step directly without further purification.

LCMS=[M+H]⁺: 476.44; Purity=50%.

### Synthesis of 1-(4-(((4-(4-morpholino-7H-pyrrolo[2,3-d]pyrimidin-6-yl)phenyl)amino)methyl)-[1,4′-bipiperidin]-1′-yl)prop-2-en-1-one (Compound 619)

To a stirred solution of N-([1,4′-bipiperidin]-4-ylmethyl)-4-(4-morpholino-7H-pyrrolo[2,3-d]pyrimidin-6-yl) aniline 61 (200 mg, 0.420 mmol) in DMF (5 mL) was added Et₃N(0.176 mL, 1.26 mmol) at 0° C. and stirred for 15 min under N₂ atmosphere. Then, acryl chloride 34 (27.2 μL, 0.336 mmol) dissolved in DMF (1 mL) was added dropwise to the reaction at 0° C. and stirred at RT for 2 h. After completion of reaction (TLC and LCMS monitoring), the reaction mixture was poured into ice cold water (50 mL) and extracted with EtOAc (3×50 mL). The combined organic layer was washed with ice cold brine (2×50 mL), dried over Na₂SO₄, filtered and concentrated under reduce pressure. The crude was purified by RP-HPLC [5-95% 0.1% TFA in water/ACN; Column: Symmetry C18 (19*300) 7μ] to get Compound 619 (16 mg, 7%).

¹H NMR (400 MHz, DMSO-d₆): δ 12.34 (s, 1H), 9.11 (s, 1H), 8.22 (s, 1H), 7.65 (d, J=8.4 Hz, 2H), 6.97 (brs, 1H), 6.85-6.79 (m, 1H), 6.64 (d, J=8.8 Hz, 2H), 6.11 (dd, J=16.8 Hz, 2.0 Hz, 1H), 5.70 (dd, J=10.4 Hz, 2.4 Hz, 1H), 4.58-4.55 (m, 1H), 4.22-4.20 (m, 1H), 3.88-3.87 (m, 4H), 3.78-3.76 (m, 4H), 3.54-3.39 (m, 3H), 3.16-2.94 (m, 5H), 2.64-2.61 (m, 1H), 2.14-1.98 (m, 4H), 1.84-1.82 (m, 1H), 1.52-1.45 (m, 2H), 1.42-1.36 (m, 2H). LCMS=[M+H]⁺: 530.49; Purity=96.84%.

### Example 16

### Compound 620

### Preparation of (E)-4-(4-(((4-(4-(3-oxa-8-azabicyclo[3.2.1]octan-8-yl)-7H-pyrrolo[2,3-d]pyrimidin-6-yl)phenyl)amino)methyl)-4-hydroxypiperidin-1-yl)-1-(3,3-difluoroazetidin-1-yl)but-2-en-1-one (Compound 620)

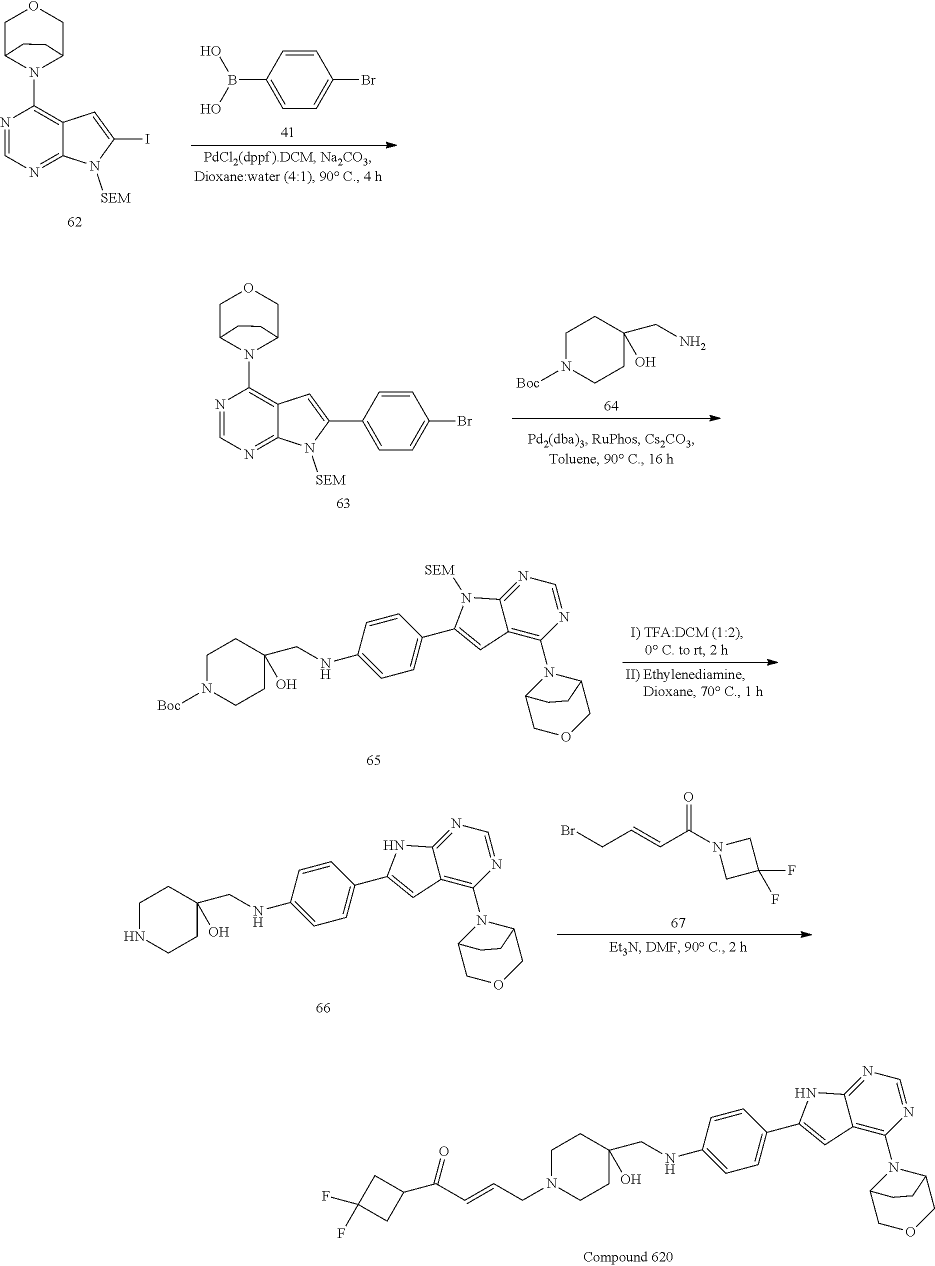

### Synthesis of (1R,5S)-8-(6-(4-bromophenyl)-7-((2-(trimethylsilyl)ethoxy)methyl)-7H-pyrrolo[2,3-d]pyrimidin-4-yl)-3-oxa-8-azabicyclo[3.2.1]octane (63)

To a stirred solution of (1R,5S)-8-(6-iodo-7-((2-(trimethylsilyl)ethoxy)methyl)-7H-pyrrolo[2,3-d]pyrimidin-4-yl)-3-oxa-8-azabicyclo[3.2.1]octane 62 (2 g, 4.11 mmol) and (4-bromophenyl) boronic acid 41 (991 mg, 4.93 mmol) in 1,4-dioxane (20 mL) was added Na₂CO₃ (1.31 g, 12.3 mmol) dissolved in water (5 mL). The resulting reaction mixture was degassed with N₂ gas for 15 min, followed by PdCl₂(dppf).DCM (336 mg, 0.411 mmol) was added to the reaction mixture and stirred at 90° C. for 4 h. After completion of reaction (TLC and LCMS monitoring), the reaction mixture was filtered through Celite pad and washed with EtOAc. The filtrate was diluted with water (50 mL) and extracted with EtOAc (3×50 mL). The combined organic layer was dried over Na₂SO₄, filtered and evaporated under reduced pressure. The crude was purified by column chromatography (silica gel 100-200 M, 24 g SNAP) using 10-15% EtOAc in heptane to get the desired compound as an off white solid 63 (1.2 g, 38%).

¹H-NMR (400 MHz, DMSO-d₆): δ8.28 (s, 1H), 7.90-7.68 (m, 4H), 7.01 (s, 1H), 5.60 (s, 2H), 4.91 (s, 2H), 3.70-3.58 (m, 6H), 2.03-1.98 (m, 4H), 0.855-0.815 (t, J=8 Hz, 2H), −0.090 (s, 9H).

LCMS: [M+H]⁺: 515.5, Purity=67%.

### Synthesis of tert-butyl 4-(((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)methyl)-4-hydroxypiperidine-1-carboxylate (65)

To a stirred solution of (1R,5S)-8-(6-(4-bromophenyl)-7-((2-(trimethylsilyl)ethoxy)methyl)-7H-pyrrolo[2,3-d]pyrimidin-4-yl)-3-oxa-8-azabicyclo[3.2.1]octane 63 (1.2 g, 2.33 mmol) and tert-butyl 4-(aminomethyl)-4-hydroxy-1-piperidinecarboxylate 64 (804 mg, 3.49 mmol) in toluene (15 mL) was added Cs₂CO₃ (2.28 g, 6.98 mmol). The resulting reaction mixture was degassed with N₂ gas for 10 min followed by, RuPhos (217 mg, 0.466 mmol) and Pd₂(dba)₃ (213 mg, 0.233 mmol) were added to the reaction mixture and stirred at 90° C. for 16 h. After the completion of reaction (TLC and LCMS monitoring), the reaction mixture was diluted with water (50 mL) and extracted with EtOAc (3×50 mL). The combined organic layer was dried over Na₂SO₄, filtered and evaporated under reduced pressure. The crude was purified by flash chromatography (silica gel 100-200 M, 12 g SNAP) using 50-55% EtOAc in heptane to get the desired compound as an off white solid 65 (850 mg, 27%).

LCMS: [M+H]⁺: 665.50; Purity=49%.

### Synthesis of 4-(((4-(4-(3-oxa-8-azabicyclo[3.2.1]octan-8-yl)-7H-pyrrolo[2,3-d]pyrimidin-6-yl)phenyl)amino)methyl)piperidin-4-ol (66)

To a stirred solution of tert-butyl 4-(((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)methyl)-4-hydroxypiperidine-1-carboxylate 65 (850 mg, 1.28 mmol) in DCM (10 mL) was added TFA (5 mL) dropwise at 0° C. The reaction mixture was stirred at RT for 2 h. After completion of reaction (TLC and LCMS monitoring), the reaction mixture was concentrated under reduced pressure. The crude was dissolved in 1,4-dioxane (6 mL) and 1,2-ethanediamine (0.128 mL, 1.92 mmol) was added to the reaction mixture and stirred at 70° C. for 1 h. After completion of reaction (LCMS monitoring), the reaction mixture was diluted with water (50 mL) and extracted with 25% IPA in CHCl₃ (3×50 mL). The combined organic layer was dried over Na₂SO₄, filtered and concentrated under reduced pressure to get the title compound as a yellow solid 66 (620 mg; crude) which was used in next step directly without further purification.

LCMS: [M+H]⁺: 435.35; Purity=55%.

### Synthesis of (E)-4-(4-(((4-(4-(3-oxa-8-azabicyclo[3.2.1]octan-8-yl)-7H-pyrrolo[2,3-d]pyrimidin-6-yl)phenyl)amino)methyl)-4-hydroxypiperidin-1-yl)-1-(3,3-difluoroazetidin-1-yl)but-2-en-1-one (Compound 620)

To a stirred solution of 4-(((4-(4-(3-oxa-8-azabicyclo[3.2.1]octan-8-yl)-7H-pyrrolo[2,3-d]pyrimidin-6-yl)phenyl)amino)methyl)piperidin-4-ol 66 (160 mg, 0.368 mmol) and (E)-4-bromo-1-(3,3-difluoro-1-azetidinyl)-2-buten-1-one 67 (106 mg, 0.442 mmol) in DMF (5 mL) was added Et₃N(0.16 mL, 1.1 mmol) at 0° C. The reaction mixture was stirred at 90° C. for 2 h. After completion of reaction (TLC and LCMS monitoring), the reaction mixture was diluted with ice cold water (50 mL) and extracted with 25% IPA in CHCl₃ (3×50 mL). The combined organic layer was dried over Na₂SO₄, filtered and evaporated under reduced pressure. The crude was purified by RP-HPLC [5-95% 0.1% TFA in water/ACN; Column: X select phenyl hexyl (19*250) 5μ] to get Compound 620 (22 mg, 10%).

¹H NMR (400 MHz, DMSO-d₆): δ 11.89 (s, 1H), 8.11 (s, 1H), 7.63 (d, J=8.8 Hz, 2H), 6.79 (d, J=1.6 Hz, 1H), 6.68 (d, J=8.4 Hz, 2H), 6.65-6.62 (m, 1H), 6.11 (d, J=15.2 Hz, 1H), 5.57 (t, J=5.6 Hz, 1H), 4.86 (brs, 2H), 4.70-4.59 (m, 2H), 4.36-4.27 (m, 3H), 3.69 (d, J=10.8 Hz, 2H), 3.60 (d, J=10.8 Hz, 2H), 3.09 (d, J=6 Hz, 2H), 3.01 (d, J=5.6 Hz, 2H), 2.34-2.30 (m, 2H), 2.02-1.94 (m, 4H), 1.62-1.52 (m, 4H) [2 protons are may merged in solvent peak].

LCMS=[M+H]⁺: 594.50; Purity=99.14%.

### Example 17

### Compound 702

### Preparation of 4-(4-(((4-(4-(3-oxa-8-azabicyclo[3.2.1]octan-8-yl)-7H-pyrrolo[2,3-d]pyrimidin-6-yl)phenyl)amino)methyl)-4-hydroxypiperidin-1-yl)-N,N-dimethylbut-2-ynamide (Compound 702)

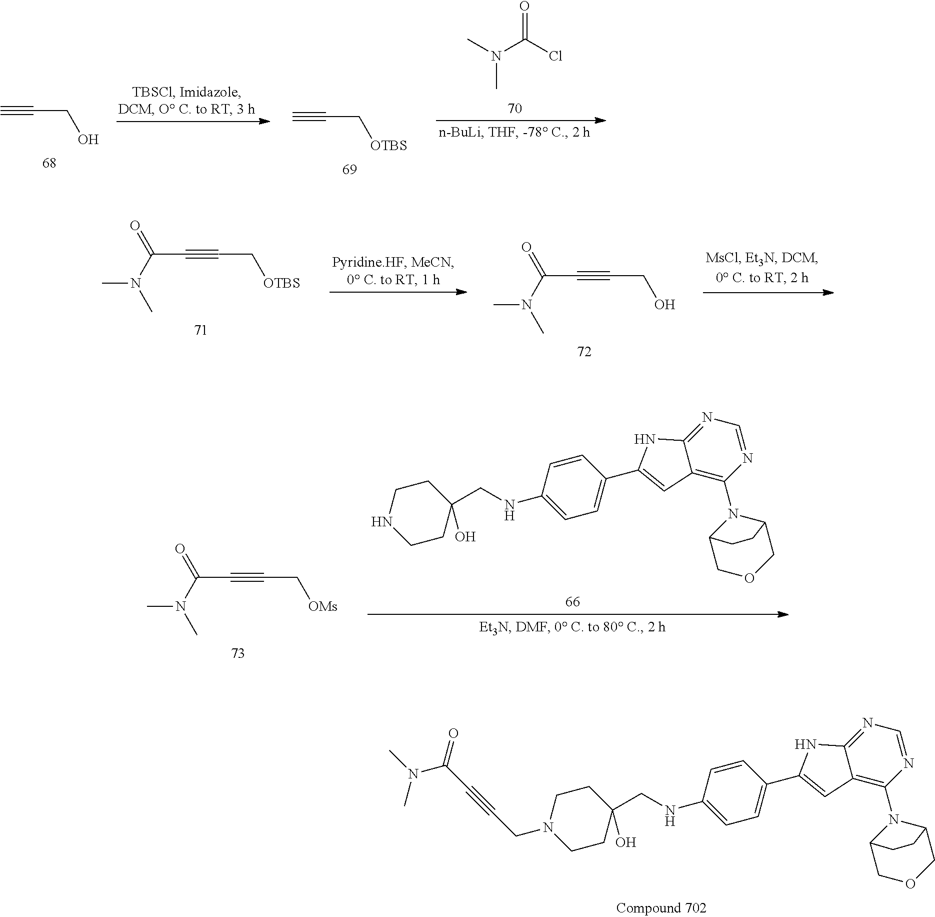

### Synthesis of (tert-butyl) bis(methyl) (2-propynyloxy) silane (69)

To an ice cold stirred solution of 2-propyn-1-ol 68 (10 g, 178 mmol) in DCM (100 mL) were added imidazole (24.3 g, 357 mmol) and TBSCl (12 g, 70.5 mmol) at under N₂ atmosphere. The reaction mixture was stirred at RT for 3 h. After completion of reaction (TLC monitoring), the reaction mixture was quenched by addition of water (150 mL) and extracted with DCM (2×100 mL). The combined organic layer was dried over Na₂SO₄, filtered and concentrated under reduced pressure. The crude was purified by flash column chromatography (silica gel 100-200 M, 120 g SNAP) using 0-5% EtOAc in heptane to get the desired compound as yellow liquid 69 (12 g, 40%) ¹H NMR (400 MHz, CDCl₃): δ 4.31 (s, 2H), 8.11 (s, 1H), 3.88 (s, 1H), 0.89 (s, 9H), 0.12 (s, 6H).

### Synthesis of N,N-dimethyl-4-[(tert-butyl) bis(methyl) siloxy]-2-butynamide (71)

To an ice cold stirred solution of (tert-butyl) bis(methyl) (2-propynyloxy) silane 69 (2 g, 11.7 mmol) in THF (25 mL) was added n-BuLi (5.16 mL, 12.9 mmol: 2.5M in hexane) dropwise at −78° C. under N₂ atmosphere. The resulting reaction mixture was stirred at same temperature for 30 min. Then, N,N-dimethylchloroformylamine 70 (1.52 g, 14.1 mmol) was added to the reaction mixture and stirred at RT for 2 h. After completion of reaction (TLC monitoring), the reaction mixture was quenched with sat. NH₄Cl (50 mL) and extracted with EtOAc (2×100 mL). The combined organic layer was dried over Na₂SO₄, filtered and concentrated under reduced pressure. The crude was purified by flash chromatography (silica gel 100-200 M, 40 g SNAP) using 25-30% EtOAc in Heptane to get the desired compound as brown oil 71 (2.3 g, 80%). ¹H NMR (400 MHz, CDCl₃): δ 4.46 (s, 2H), 3.20 (s, 3H), 2.97 (s, 3H), 0.90 (s, 9H), 0.12 (s, 6H).

LCMS: [M+H]⁺: 242.30; Purity=98.70%.

### Synthesis of N,N-dimethyl-4-hydroxy-2-butynamide (72)

To a stirred solution of N,N-dimethyl-4-[(tert-butyl) bis(methyl) siloxy]-2-butynamide 71 (2.3 g, 9.53 mmol) in ACN(25 mL) was added pyridine.HF (4.3 mL, 47.6 mmol) dropwise at 0° C. The resulting reaction mixture was stirred at RT for 1 h. After completion of reaction (TLC and LCMS monitoring), the resulting reaction mixture was quenched with sat. NaHCO₃ and extracted with DCM (3×50 mL). The combined organic layer was dried over Na₂SO₄, filtered and evaporated under reduced pressure to get the desired compound as yellow oil 72 (1.9 g; crude) which was used in next step directly without further purification. ¹H NMR (400 MHz, CDCl₃): $4.43 (s, 2H), 3.21 (s, 3H), 2.98 (s, 3H).

LCMS: [M+H]⁺: 128.02; Purity=98%.

### Synthesis of N,N-dimethyl-4-(mesyloxy)-2-butynamide (73)

To an ice cold stirred solution of N,N-dimethyl-4-hydroxy-2-butynamide 72 (800 mg, 6.29 mmol) in DCM (10 mL) were added Et₃N(4.36 mL, 31.5 mmol) and MsCl (1.46 mL, 18.9 mmol) under N₂ atmosphere. The resulting reaction mixture was stirred at RT for 2 h. After completion of reaction (TLC and LCMS monitoring), the reaction mixture was diluted with ice cold water (50 mL) and extracted with DCM (3×50 mL). The combined organic layer was dried over Na₂SO₄, filtered and evaporated under reduced pressure. The crude was purified by flash chromatography (silica gel 100-200 M, 12 g SNAP) using 60-65% EtOAc in heptane to get the desired compound as yellow oil 72 (800 mg, 26%).

¹H NMR (400 MHz, CDCl₃): δ 4.98 (s, 2H), 3.19 (s, 3H), 3.14 (s, 3H), and 2.98 (s, 3H).

LCMS: [M+H]: 206.14; Purity=99.39%.

### Synthesis of N,N-dimethyl-4-{4-hydroxy-4-[(p-{4-(3-oxa-8-azabicyclo[3.2.1]oct-8-yl)-1H-1,5,7-triazainden-2-yl}phenylamino)methyl]-1-piperidyl}-2-butynamide (Compound 702)

To a stirred solution of 4-[(p-{4-(3-oxa-8-azabicyclo[3.2.1]oct-8-yl)-1H-1,5,7-triazainden-2-yl}phenylamino)methyl]-4-piperidinol 66 (200 mg, 0.460 mmol) and N,N-dimethyl-4-(mesyloxy)-2-butynamide 72 (94.5 mg, 0.460 mmol) in DMF (5 mL) was added Et₃N(0.2 mL, 1.38 mmol) at 0° C. under N₂ atmosphere. The resulting reaction mixture was stirred at 80° C. for 2 h. After completion of reaction (TLC and LCMS monitoring), the reaction mixture was diluted with ice cold water (50 mL) and extracted with 25% IPA in CHCl₃ (3×50 mL). The combined organic layer was dried over Na₂SO₄, filtered and evaporated under reduced pressure. The crude was purified by RP-HPLC [5-95% 0.1% TFA in water/ACN; Column: symmetry C18 (19*300) 7μ] to get Compound 702 (20 mg, 8%).

¹H NMR (400 MHz, DMSO-d₆): δ 12.55 (brs, 1H), 10.09 (brs, 1H), 8.25 (s, 1H), 7.67 (d, J=8.4 Hz, 2H), 7.05 (s, 1H), 6.72 (d, J=8.4 Hz, 2H), 5.94-5.90 (m, 1H), 4.96 (s, 2H), 4.41 (s, 2H), 3.76-3.67 (m, 5H), 3.43-3.38 (m, 2H), 3.29-3.20 (m, 2H), 3.16 (s, 4H), 2.87 (s, 3H), 2.12-2.05 (m, 4H), 1.90-1.78 (m, 4H). LCMS=[M+H]⁺: 544.53; Purity=99.74%.

### Example 18

### Compound 703

### Preparation of 4-(4-(1-((4-(4-(3-oxa-8-azabicyclo[3.2.1]octan-8-yl)-7H-pyrrolo]2,3-d]pyrimidin-6-yl)phenyl)amino)-2,2,2-trifluoroethyl)piperidin-1-yl)-N,N-dimethylbut-2-ynamide (Compound 703)

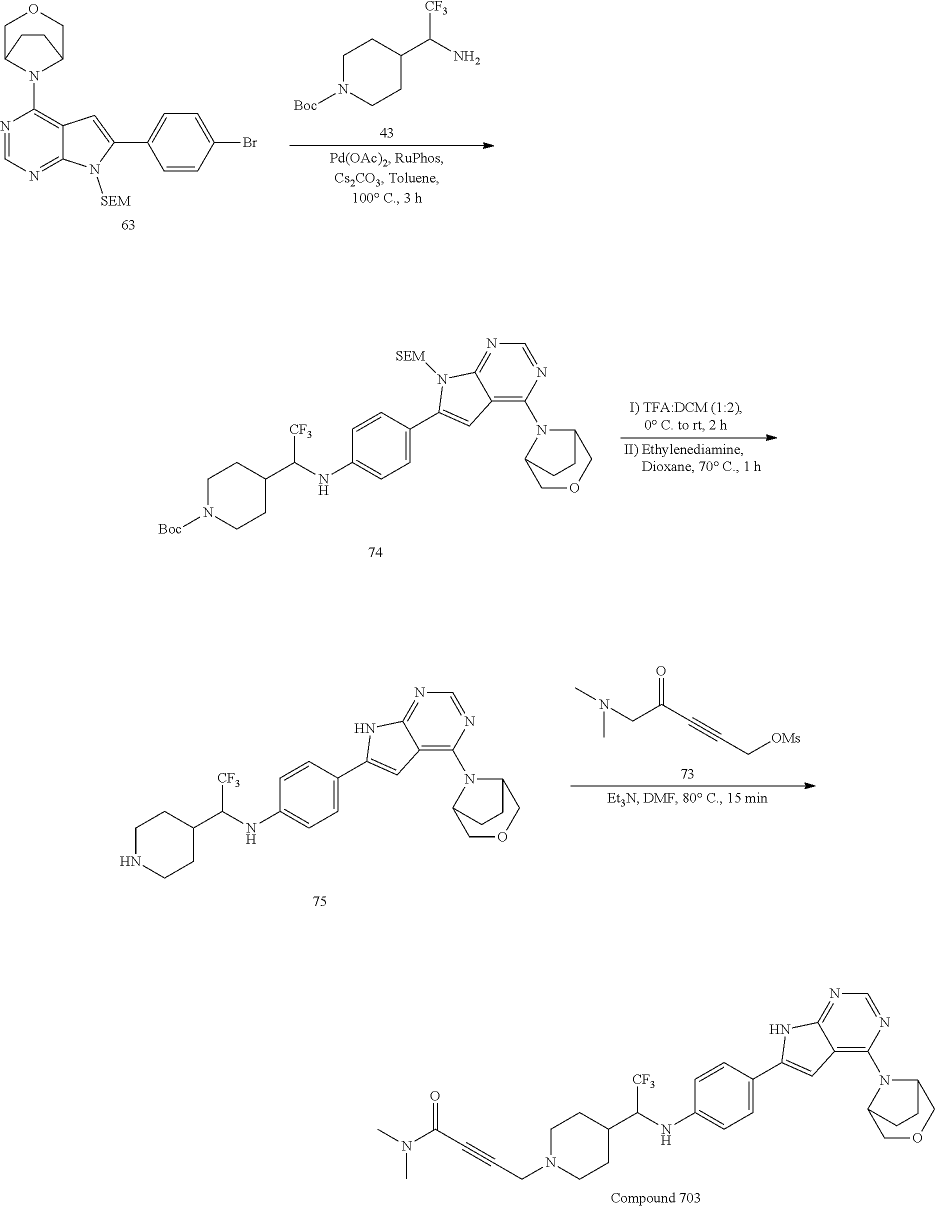 [SMILES: C[Si](C)(C)CCOCn1c(-c2ccc(Br)cc2)cc2c(N3C4CCC3COC4)ncnc21 ; CC(C)(C)OC(=O)N1CCC(C(N)C(F)(F)F)CC1 ; CC(C)(C)OC(=O)N1CCC(C(Nc2ccc(-c3cc4c(N5C6CCC5COC6)ncnc4n3COCC[Si](C)(C)C)cc2)C(F)(F)F)CC1 ; FC(F)(F)C(Nc1ccc(-c2cc3c(N4C5CCC4COC5)ncnc3[nH]2)cc1)C1CCNCC1 ; CN(C)CC(=O)C#CCOS(C)(=O)=O ; CN(C)C(=O)C#CCN1CCC(C(Nc2ccc(-c3cc4c(N5C6CCC5COC6)ncnc4[nH]3)cc2)C(F)(F)F)CC1]

### Synthesis of tert-butyl 4-(1-((4-(4-(3-oxa-8-azabicyclo[3.2.1]octan-8-yl)-7-((2-(trimethylsilyl)ethoxy)methyl)-7H-pyrroslo[2,3-d]pyrimidin-6-yl)phenyl)amino)-2,2,2-trifluoroethyl)piperidine-1-carboxylate (74)

To a stirred solution of [2-({2-(p-bromophenyl)-4-(3-oxa-8-azabicyclo[3.2.1]oct-8-yl)-1H-1,5,7-triazainden-1-yl}methoxy)ethyl]tris(methyl) silane 63 (500 mg, 0.970 mmol) and tert-butyl 4-(1-amino-2,2,2-trifluoroethyl)-1-piperidinecarboxylate 43 (383 mg, 1.36 mmol) in toluene (10 mL) was added Cs₂CO₃ (948 mg, 2.91 mmol) at RT. The resulting reaction mixture was degassed with N₂ gas for 10 min. After that, RuPhos (90.5 mg, 0.194 mmol) and Pd(OAc)₂ (21.8 mg, 0.097 mmol) were added to the reaction mixture and stirred at 100° C. for 3 h. After completion of reaction (TLC and LCMS monitoring), the reaction mixture was quenched with water (50 mL) and extracted with EtOAc (3×50 mL). The combined organic layer was washed with brine solution (2×50 mL), dried over Na₂SO₄, filtered and concentrated under reduced pressure. The crude was purified by flash chromatography (silica gel 100-200 M, 12 g SNAP) using 25-30% EtOAc in heptane to get the title compound as a yellow solid 74 (700 mg, 50%).

LCMS=[M+H]⁺: 717.92; Purity=55%.

### Synthesis of 4-(4-(3-oxa-8-azabicyclo[3.2.1]octan-8-yl)-7H-pyrrolo[2,3-d]pyrimidin-6-yl)-N-(2,2,2-trifluoro-1-(piperidin-4-yl)ethyl) aniline (75)

To a stirred solution of tert-butyl 4-(2,2,2-trifluoro-1-{p-[4-(3-oxa-8-azabicyclo[3.2.1]oct-8-yl)-1-{[2-(trimethylsilyl)ethoxy]methyl}-1H-1,5,7-triazainden-2-yl]phenylamino}ethyl)-1-piperidinecarboxylate 74 (700 mg, 0.976 mmol) in DCM (10 mL) was added TFA (5 mL) dropwise at 0° C. and stirred at RT for 2 h. After completion of reaction (TLC and LCMS monitoring), the reaction mixture was concentrated under reduced pressure and triturated with Et₂O and dried under reduced pressure. The crude was dissolved in 1,4-dioxane (10 mL), followed by, 1,2-ethanediamine (0.32 mL, 4.88 mmol) was added and the reaction mixture was stirred at 70° C. for 1 h. After completion of the reaction (monitoring by LCMS), the reaction was diluted with water (20 mL) and extracted with 25% IPA in CHCl₃ (3×50 mL), dried over Na₂SO₄, filtered and concentrated under reduced pressure to get the title compound as a yellow semi solid 75 (400 mg; crude) which was used in next step directly without further purification.

LCMS=[M+H]⁺: 487.54; Purity=55%.

### Synthesis of 4-(4-(1-((4-(4-(3-oxa-8-azabicyclo[3.2.1]octan-8-yl)-7H-pyrrolo[2,3-d]pyrimidin-6-yl)phenyl)amino)-2,2,2-trifluoroethyl)piperidin-1-yl)-N,N-dimethylbut-2-ynamide (Compound 703)

To a stirred solution of [2,2,2-trifluoro-1-(4-piperidyl)ethyl](p-{4-(3-oxa-8-azabicyclo[3.2.1]oct-8-yl)-1H-1,5,7-triazainden-2-yl}phenyl) amine 75 (350 mg, 0.719 mmol) in DMF (5 mL) were added N,N-dimethyl-4-(mesyloxy)-2-butynamide 73 (177 mg, 0.863 mmol) and Et₃N(0.301 mL, 2.16 mmol) at RT and the reaction mixture was stirred at 80° C. for 15 min. After completion of reaction (monitoring by TLC and LCMS), the reaction mixture was quenched with ice cold water (10 mL) and extracted with EtOAc (3×50 mL). The combined organic layer was washed with ice cold brine (2×50 mL), dried over Na₂SO₄, filtered and concentrated under reduced pressure. The crude was purified by RP-HPLC [5-95% 5 mM ammonium bicarbonate in Water/ACN; Column: X Select Phenyl Hexyl (19*250) 5μ] to obtain Compound 703 (100 mg, 23%).

¹H NMR (400 MHz, DMSO-d₆): δ 11.95 (s, 1H), 8.13 (s, 1H), 7.65 (d, J=8.8 Hz, 2H), 6.86-6.88 (m, 3H), 6.08 (d, J=8.8 Hz, 1H), 4.87 (s, 2H), 4.30-4.26 (m, 1H), 3.69 (d, J=10.4 Hz, 2H), 3.61 (d, J=10.4 Hz, 2H), 3.49 (brs, 2H), 3.14 (s, 3H), 2.84 (s, 5H), 2.18-2.12 (m, 2H), 2.03-2.01 (m, 2H), 1.97-1.94 (m, 2H), 1.79-1.75 (m, 3H), 1.48-1.42 (m, 2H).

LCMS=[M+H]⁺: 596.67; Purity=96.95%.

### Example 19

### Compound 622

### Preparation of 4-((3-acrylamidoazetidin-1-yl)methyl)-N-(4-(4-morpholino-7H-pyrrolo[2,3-d]pyrimidin-6-yl)phenyl)picolinamide (Compound 622)

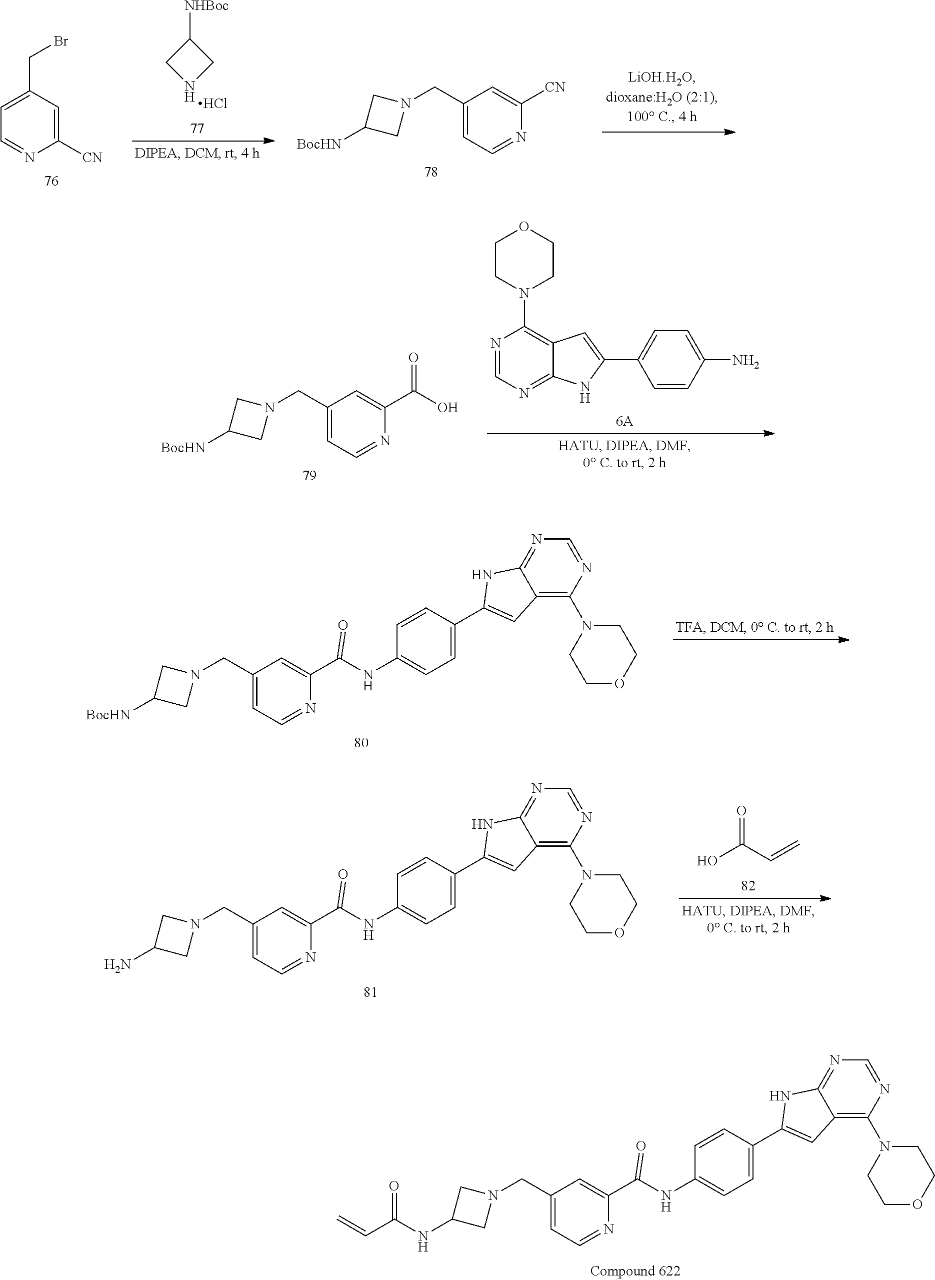

### Synthesis of tert-butyl(1-((2-cyanopyridin-4-yl)methyl)azetidin-3-yl)carbamate (78)

To a stirred solution of tert-butyl 3-azetidinylcarbamate 77 (2 g, 11.6 mmol) in DCM (40 mL) were added 4-(bromomethyl)-2-pyridinecarbonitrile 76 (3.43 g, 17.4 mmol) and DIPEA (6.07 mL, 34.8 mmol) sequentially and the reaction mixture was stirred at RT for 4 h. After completion of reaction (TLC monitoring), the reaction mixture was diluted with ice-cold water (50 mL) and extracted with DCM (2×100 mL). The combined organic layer was washed with brine solution (2×50 mL), dried over Na₂SO₄, filtered and evaporated under reduced pressure. The crude was purified by flash chromatography (silica gel 100-200 M, 40 g SNAP) using 30-40% EtOAc in heptane to get the title compound as brown viscous liquid 78 (1.2 g, 35%).

LCMS: [M+H]⁺: 289.09; Purity=78%.

### Synthesis of 4-((3-((tert-butoxycarbonyl)amino) azetidin-1-yl)methyl)picolinic acid (79)

To a stirred solution of 1-[(2-cyano-4-pyridyl)methyl]-3-azetidinyl 2-methyl-2-propanecarbamate 78 (1.2 g, 4.16 mmol) in 1,4-dioxane (20 mL) was added LiOH·H₂O (2.1 g, 49.9 mmol) dissolved in water (10 mL) at RT. The resulting reaction mixture was stirred at 100° C. for 4 h. After completion of reaction (LCMS monitoring), the reaction mixture was concentrated under reduced pressure. The crude was dissolved in 25% IPA in CHCl₃ (50 mL) and stirred for few minutes then filtered through sintered funnel. The filtrate was concentrated under reduced pressure to get the title compound as an off white solid 79 (1 g; as Lit salt) crude, which was used in next step directly without any purification.

LCMS: [M+H]⁺: 308.40; Purity=60%.

### Synthesis of tert-butyl(1-((2-((4-(4-morpholino-7H-pyrrolo[2,3-d]pyrimidin-6-yl)phenyl)carbamoyl)pyridin-4-yl)methyl)azetidin-3-yl)carbamate (80)

To a stirred solution of 4-(4-morpholino-7H-pyrrolo[2,3-d]pyrimidin-6-yl) aniline 6A (500 mg, 1.69 mmol) and 4-((3-((tert-butoxycarbonyl)amino) azetidin-1-yl)methyl)picolinic acid 79 (636 mg, 2.03 mmol) in DMF (10 mL) were added DIPEA (0.9 mL, 5.08 mmol) and HATU (1.29 g, 3.39 mmol) respectively at 0° C. The reaction mixture was stirred at RT for 2 h. After completion of reaction (monitoring by TLC), the reaction mixture was poured into ice cold water (50 mL) and extracted with 25% IPA in CHCl₃ (3×50 mL). The combined organic layer was over dried over Na₂SO₄, filtered and concentrated under reduced pressure. The crude was purified by flash chromatography (silica gel 100-200 M, 12 g SNAP) using 35-40% EtOAc in heptane to get the desired product as brown viscous liquid 80 (700 mg, 70%).

LCMS: [M+H]⁺: 715.65; Purity=65%

### Synthesis of 4-((3-aminoazetidin-1-yl)methyl)-N-(4-(4-morpholino-7H-pyrrolo[2,3-d]pyrimidin-6-yl)phenyl)picolinamide (81)

To a stirred solution of tert-butyl(1-((2-((4-(4-morpholino-7-((2-(trimethylsilyl)ethoxy)methyl)-7H-pyrrolo[2,3-d]pyrimidin-6-yl)phenyl)carbamoyl)pyridin-4-yl)methyl)azetidin-3-yl)carbamate 80 (700 mg, 1.2 mmol) in DCM (10 mL) was added TFA (5 mL) dropwise at 0° C. The reaction mixture was stirred at RT for 2 h. After completion of reaction (LCMS monitoring), the reaction mixture was quenched with NaHCO₃ and extracted with 25% IPA in CHCl₃ (3×50 mL). The combined organic layer was dried over Na₂SO₄, filtered and concentrated under reduced pressure to get the title compound as an off white solid 81 (450 mg; crude) which was used in next step directly without further purification.

LCMS: [M+H]⁺: 485.39; Purity=50%

### Synthesis of 4-((3-acrylamidoazetidin-1-yl)methyl)-N-(4-(4-morpholino-7H-pyrrolo[2,3-d]pyrimidin-6-yl)phenyl)picolinamide (Compound 622)

To a stirred solution of 4-((3-aminoazetidin-1-yl)methyl)-N-(4-(4-morpholino-7H-pyrrolo[2,3-d]pyrimidin-6-yl)phenyl)picolinamide 81 (300 mg, 0.619 mmol) and acrylic acid 82 (67 mg, 0.929 mmol) in DMF (5 mL) were added DIPEA (0.32 mL, 1.86 mmol) and HATU (471 mg, 1.24 mmol) sequentially at 0° C. and the reaction mixture was stirred at RT for 2 h. After completion of reaction (monitoring by TLC), the reaction mixture was poured into ice cold water (50 mL) and extracted with 25% IPA in CHCl₃ (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5u)] to get Compound 622 (8 mg, 2.4%).

¹H NMR (400 MHz, DMSO-d₆): δ 12.20 (s, 1H), 10.73 (s, 1H), 8.67 (d, J=4.8 Hz, 1H), 8.61 (d, J=7.6 Hz, 1H), 8.18 (s, 1H), 8.08 (brs, 1H), 7.99 (d, J=8.8 Hz, 2H), 7.91 (d, J=8.8 Hz, 2H), 7.58 (d, J=4.8 Hz, 1H), 7.16 (d, J=2.0 Hz, 1H), 6.25-6.18 (m, 1H), 6.09 (dd, J=17.2 Hz, 2.4 Hz, 1H), 5.61 (dd, J=10 Hz, 2 Hz, 1H), 4.43-4.38 (m, 1H), 3.90-3.87 (m, 4H), 3.76-3.74 (m, 6H), 3.66-3.58 (m, 2H), 3.11-3.08 (m, 2H).

LCMS: [M+H]⁺: 539.33; Purity=97.29%.

### Example 20

### Compound 623

### Preparation of (R)—N-(1-(5-((4-(4-morpholino-7H-pyrrolo[2,3-d]pyrimidin-6-yl)phenyl)amino)pyrimidin-2-yl)piperidin-3-yl)acrylamide (Compound 623)

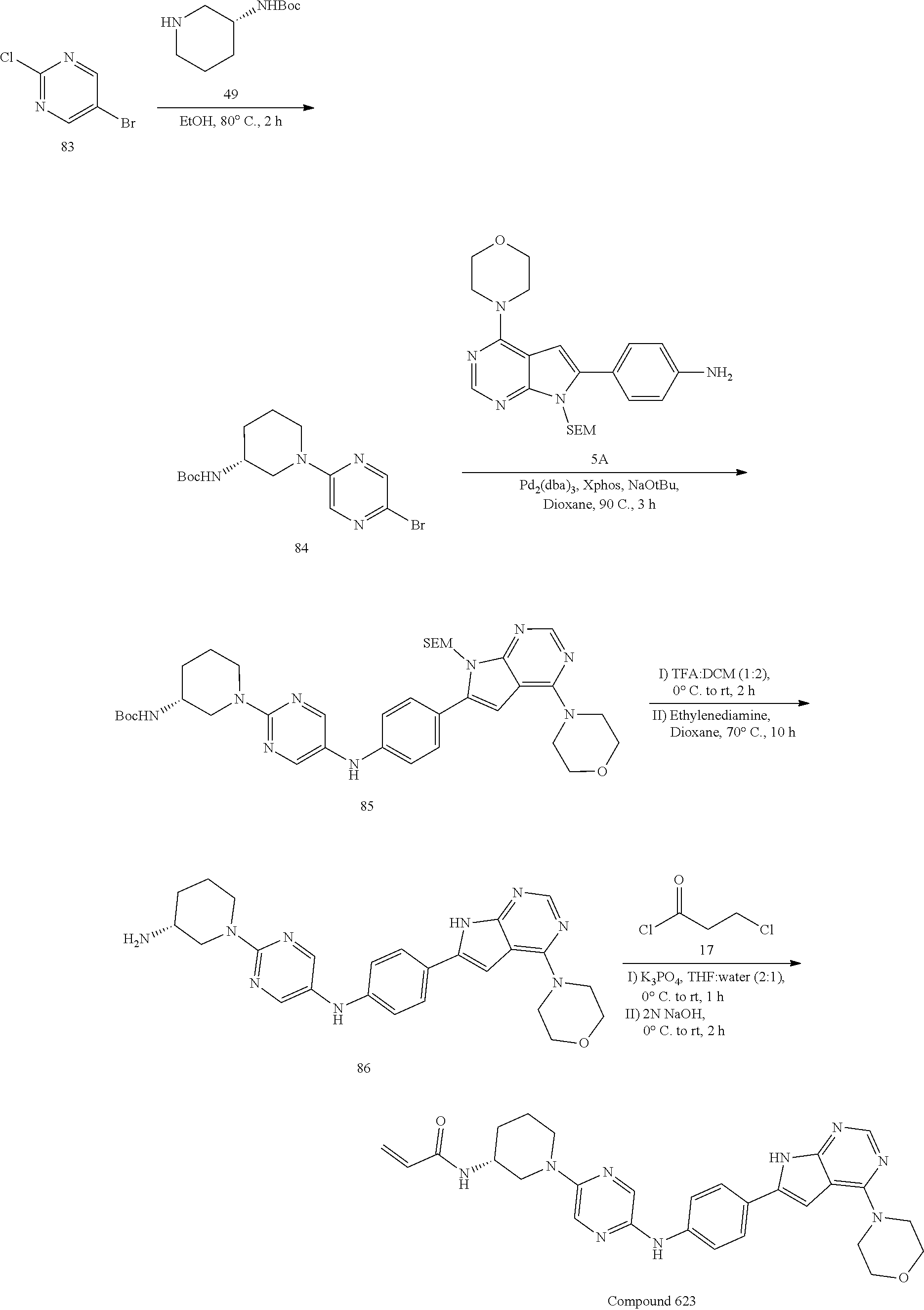

### Synthesis of tert-butyl(R)-(1-(5-bromopyrimidin-2-yl)piperidin-3-yl)carbamate (84)

To a stirred solution of 5-bromo-2-chloropyrimidine 83 (500 mg, 2.58 mmol) in EtOH (10 mL) was added and (R)-3-piperidylamino-tert-butylformylate 49 (1.04 g, 5.17 mmol) and the reaction mixture was stirred at 80° C. for 2 h. After completion of reaction (TLC monitoring), the reaction mixture was concentrated under reduced pressure. The crude was diluted with water (50 mL) and extracted with EtOAc (3×50 mL). The combined organic layer was dried over Na₂SO₄, filtered and evaporated under reduced pressure to get the title compound as a brown solid 84 (700 mg, 76%).

¹H NMR (400 MHz, DMSO-d₆) § 8.42 (s, 2H), 6.91 (d, J=7.6 Hz, 1H), 4.42 (d, J=12.4 Hz, 1H), 4.29 (d, J=12 Hz, 1H), 2.93 (t, J=9.6 Hz 1H), 2.83-2.80 (m, 1H), 1.85-1.81 (m, 1H), 1.77-1.70 (m, 1H), 1.40-1.36 (m, 12H).

LCMS=[M+H]⁺: 357.24; Purity=93%.

### Synthesis of tert-butyl(R)-(1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl)carbamate (85)

To a stirred solution of tert-butyl[(R)-1-(5-bromo-2-pyrimidinyl)-3-piperidyl]carbamate 84 (800 mg, 2.24 mmol) and p-(4-morpholino-1-{[2-(trimethylsilyl)ethoxy]methyl}-1H-1,5,7-triazainden-2-yl) aniline 5A (953 mg, 2.24 mmol) in 1,4-dioxane (12 mL) was added NaO Bu (323 mg, 3.36 mmol) at RT. The reaction mixture was degassed with N₂ gas for 10 min. After that, Xphos (107 mg, 224 mmol) and Pd₂(dba)₃ (181 mg, 0.224 mmol) were added to the reaction mixture and stirred at 90° C. for 3 h. After the completion of reaction (TLC and LCMS monitoring), the reaction mixture was diluted with water (50 mL) and extracted with EtOAc (3×50 mL). The combined organic layer was dried over Na₂SO₄, filtered and concentrated under reduced pressure. The crude was purified by flash chromatography (silica gel 100-200 M, 12 g SNAP) using 50-55% EtOAc in heptane to get the title compound as an off white solid 85 (800 mg, 35%).

LCMS=[M+H]⁺: 702.45; Purity=86%.

### Synthesis of (R)-2-(3-aminopiperidin-1-yl)-N-(4-(4-morpholino-7H-pyrrolo[2,3-d]pyrimidin-6-yl)phenyl)pyrimidin-5-amine (86)

To a stirred solution of tert-butyl[(R)-1-{5-[p-(4-morpholino-1-{[2-(trimethylsilyl)ethoxy]methyl}-1H-1,5,7-triazainden-2-yl)phenylamino]-2-pyrimidinyl}-3-piperidyl]carbamate 58 (800 mg, 1.14 mmol) in DCM (5 mL) was added TFA (2.5 mL) dropwise at 0° C. The reaction mixture was stirred at RT for 2 h. After completion of SM (TLC monitoring), the reaction mixture was concentrated under reduced pressure. The crude was dissolved in 1,4-dioxane (10 mL), followed by 1,2-ethanediamine (0.23 mL, 3.42 mmol) was added to the reaction mixture and stirred at 70° C. for 1 h. After completion of reaction (LCMS monitoring), the reaction mixture diluted with water (50 mL) and extracted with 25% IPA in CHCl₃ (3×50 mL). The combined organic layer was dried over Na₂SO₄, filtered and concentrated under reduced pressure to get the desired compound as an off white solid 86 (400 mg; crude) which was used in next step directly without further purification.

LCMS=[M+H]⁺: 472.32; Purity=79%.

### Synthesis of (R)—N-(1-(5-((4-(4-morpholino-7H-pyrrolo[2,3-d]pyrimidin-6-yl)phenyl)amino)pyrimidin-2-yl) piperidin-3-yl)acrylamide (Compound 623)

To a stirred solution of 2-[(R)-3-amino-1-piperidyl]-5-[p-(4-morpholino-1H-1,5,7-triazainden-2-yl)phenylamino]pyrimidine 86 (400 mg, 0.848 mmol) in THF (10 mL) was added K₃PO₄ (360 mg, 1.7 mmol) dissolved in water (4 mL) at 0° C. Then, 3-chloropropionyl chloride 17 (97.2 μL, 1.02 mmol) dissolved in THF (1 mL) was added to the reaction mixture and stirred at RT for 1 h. After consumed of SM (TLC monitoring), 2N aq. NaOH (5 mL, 10.2 mmol) was added dropwise at 0° C. and stirred at RT for next 2 h. After completion of reaction (LCMS monitoring), the reaction mixture was diluted with water (50 mL) and extracted with 2-MeTHF (3×50 mL). The combined organic layer was dried over Na₂SO₄, filtered and concentrated under reduced pressure. The crude residue was purified by RP-HPLC [5-95% 0.1% TFA in water/ACN; Column: X select phenyl hexyl column (19*250) 5μ] to get Compound 623 (105 mg, 23%).

¹H NMR (400 MHz, DMSO-d₆): δ 12.47 (brs, 1H), 8.29 (s, 2H), 8.24 (s, 1H), 8.10 (d, J=8.0 Hz, 1H), 7.95 (brs, 1H), 7.71 (d, J=8.8 Hz, 2H), 7.07 (brs, 1H), 6.82 (d, J=8.4 Hz, 2H), 6.28-6.21 (m, 1H), 6.12 (dd, J=16.8 Hz. 2.4 Hz, 1H), 5.59 (dd, J=10 Hz, 2.4 Hz, 1H), 4.46-4.42 (m, 1H), 4.31-4.28 (m, 1H), 4.04-3.97 (m, 4H), 3.91-3.98 (m, 5H), 3.10-3.05 (m, 1H), 2.95-2.89 (m, 1H), 1.93-1.89 (m, 1H), 1.80-1.77 (m, 1H), 1.51-1.46 (m, 2H).

LCMS=[M+H]⁺: 526.47; Purity=99.58%.

### Example 21

### Compound 624

### Preparation of 1-(4-(((4-(4-(3-oxa-8-azabicyclo[3.2.1]octan-8-yl)-7H-pyrrolo[2,3-d]pyrimidin-6-yl)phenyl)amino)methyl)-4-fluoropiperidin-1-yl)prop-2-en-1-one (Compound 624)

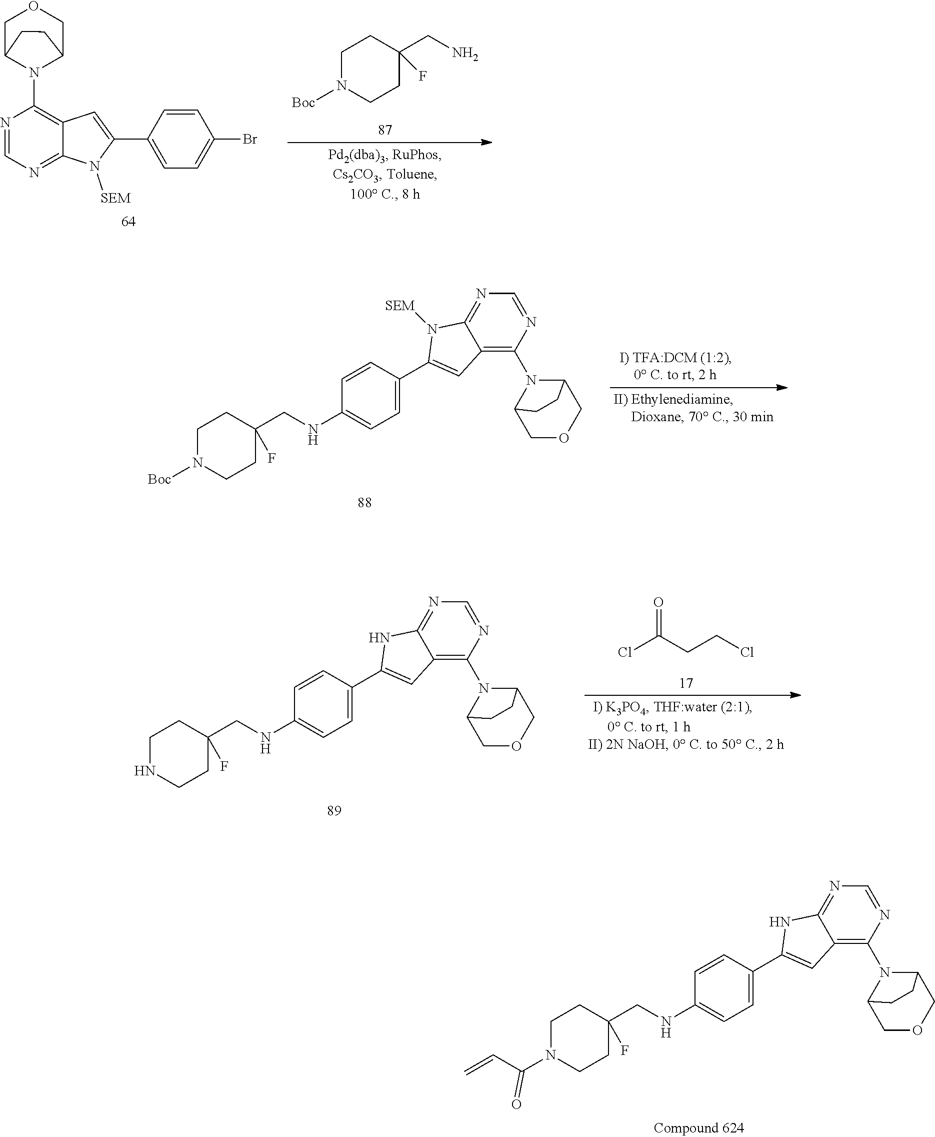 [SMILES: C[Si](C)(C)CCOCn1c(-c2ccc(Br)cc2)cc2c(N3C4CCC3COC4)ncnc21 ; CC(C)(C)OC(=O)N1CCC(F)(CN)CC1 ; CC(C)(C)OC(=O)N1CCC(F)(CNc2ccc(-c3cc4c(N5C6CCC5COC6)ncnc4n3COCC[Si](C)(C)C)cc2)CC1 ; FC1(CNc2ccc(-c3cc4c(N5C6CCC5COC6)ncnc4[nH]3)cc2)CCNCC1 ; O=C(Cl)CCCl ; C=CC(=O)N1CCC(F)(CNc2ccc(-c3cc4c(N5C6CCC5COC6)ncnc4[nH]3)cc2)CC1]

### Synthesis of tert-butyl 4-(((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)methyl)-4-fluoropiperidine-1-carboxylate (88)

To a stirred solution of (1R,5S)-8-(6-(4-bromophenyl)-7-((2-(trimethylsilyl)ethoxy)methyl)-7H-pyrrolo[2,3-d]pyrimidin-4-yl)-3-oxa-8-azabicyclo[3.2.1]octane 64 (500 mg, 0.970 mmol) and tert-butyl 4-(aminomethyl)-4-fluoropiperidine-1-carboxylate 87 (338 mg, 1.45 mmol) in toluene (10 mL) was added Cs₂CO₃ (948 mg, 2.91 mmol) and the resulting reaction mixture was degassed with N₂ gas for 10 min. After that, RuPhos (90.5 mg, 0.194 mmol) and Pd₂(dba)₃ (78.5 mg, 0.097 mmol) were added sequentially to the reaction mixture and stirred at 100° C. for 8 h. After completion of reaction (TLC and LCMS monitoring), the reaction mixture was diluted with water (50 mL) and extracted with EtOAc (3×50 mL). The combined organic layer was dried over Na₂SO₄, filtered and concentrated under reduced pressure. The crude was purified by flash chromatography (silica gel 100-200 M, 12 g SNAP) using 45-50% EtOAc in heptane to get the desired compound as a yellow sticky solid 88 (700 mg, 33%) LCMS=[M+H]⁺: 666.37

### Synthesis of 4-(4-(3-oxa-8-azabicyclo[3.2.1]octan-8-yl)-7H-pyrrolo[2,3-d]pyrimidin-6-yl)-N-((4-fluoropiperidin-4-yl)methyl) aniline (89)

To a stirred solution of tert-butyl 4-(((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)methyl)-4-fluoropiperidine-1-carboxylate 88 (650 mg, 0.975 mmol) in DCM (6 mL) was added TFA (3 mL) dropwise at 0° C. and the reaction mixture was stirred at RT for 2 h. After completion of reaction (TLC monitoring), the reaction mixture was concentrated under reduced pressure. The crude was dissolved in 1,4-dioxane (5 mL) followed by, 1,2-ethanediamine (0.13 mL, 1.95 mmol) was added and the reaction mixture was stirred at 70° C. for 30 min. After completion of reaction (LCMS monitoring), the reaction mixture was diluted with water (50 mL) and extracted with 25% IPA in CHCl₃ (3×50 mL). The combined organic layer was dried over Na₂SO₄, filtered and concentrated under reduced pressure to get the desired compound as a brown solid 89 (400 mg; crude) which was used in next step directly without further purification.

LCMS=[M+H]⁺: 436.24

### Synthesis of 1-(4-(((4-(4-(3-oxa-8-azabicyclo[3.2.1]octan-8-yl)-7H-pyrrolo[2,3-d]pyrimidin-6-yl)phenyl)amino)methyl)-4-fluoropiperidin-1-yl)prop-2-en-1-one (Compound 624)

To a stirred solution of 4-(4-(3-oxa-8-azabicyclo[3.2.1]octan-8-yl)-7H-pyrrolo[2,3-d]pyrimidin-6-yl)-N-((4-fluoropiperidin-4-yl)methyl) aniline 89 (400 mg, 0.916 mmol) in THF (8 mL) was added K₃PO₄ (389 mg, 1.83 mmol) dissolved in water (4 mL) at 0° C. Then, 3-chloropropionyl chloride 17 (140 mg, 1.1 mmol) was added dropwise to the reaction mixture at 0° C. and stirred at RT for 1 h. Then, 2M aq. NaOH (5.5 mL, 11 mmol) was added dropwise to the reaction mixture and stirred at 50° C. for 2 h. After completion of reaction (TLC and LCMS monitoring), the reaction mixture was poured into ice cold water (50 mL) and extracted with 2-MeTHF (3×50 mL). The combined organic layer was dried over Na₂SO₄, filtered and concentrated under reduced pressure. The crude was purified by flash column chromatography (silica gel 100-200 M, 12 g SNAP) using 7-10% MeOH in DCM to get the title compound. To enrich the purity, the compound was again purified by RP-HPLC [5-95% 5 mM ammonium bicarbonate in Water/ACN; Column: X SCLECT PHENYL HEXYL (19*250) 5μ] to get Compound 624 (20 mg, 4.5%) ¹H NMR (400 MHz, DMSO-d₆): δ 11.90 (s, 1H), 8.12 (s, 1H), 7.62 (d, J=8.4 Hz, 2H), 6.86-6.79 (m, 2H), 6.72 (d, J=8.8 Hz, 2H), 6.10 (dd, J=16.4 Hz, 2.4 Hz, 1H), 5.99 (t, J=6.4 Hz, 1H), 5.67 (dd, J=10.4 Hz, 2.4 Hz, 1H), 4.86 (brs, 2H), 4.30-4.27 (m, 1H), 3.98-3.95 (m, 1H), 3.69 (d, J=10.8 Hz, 2H), 3.60 (d, J=10.4 Hz, 2H), 3.37-3.36 (m, 2H), 3.27-3.24 (m, 1H), 2.91 (t, J=12.4 Hz, 1H), 2.04-1.96 (m, 4H), 1.94-1.85 (m, 2H), 1.77-1.59 (m, 2H).

LCMS=[M+H]⁺: 491.44; Purity=98.45%.

### Example 22

### Compound 625

### Preparation of N-((3R)-1-((2-(1-((4-(4-(3-oxa-8-azabicyclo[3.2.1]octan-8-yl)-7H-pyrrolo]2,3-d]pyrimidin-6-yl)phenyl)amino)ethyl)pyridin-4-yl)methyl)piperidin-3-yl)acrylamide (Compound 625)

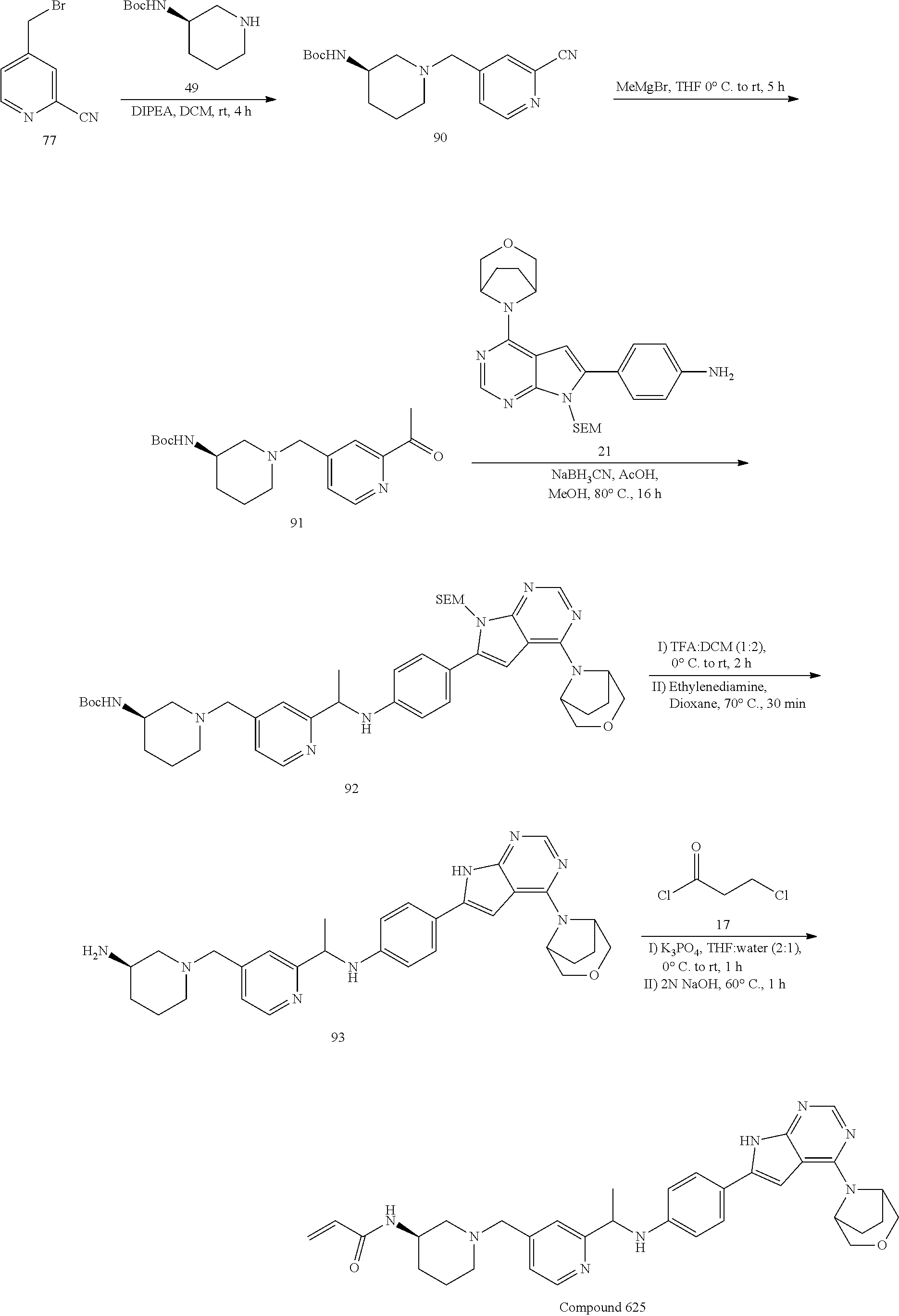

### Synthesis of tert-butyl(R)-(1-((2-cyanopyridin-4-yl)methyl)piperidin-3-yl)carbamate (90)

To a stirred solution of 4-(bromomethyl)-2-pyridinecarbonitrile 77 (10 g, 25.4 mmol) in DCM (50 mL) were added (R)-3-piperidylamino-tert-butylformylate 49 (5.08 g, 25.4 mmol) and DIPEA (13.3 mL, 76.1 mmol) at RT and stirred for 4 h. After completion of reaction (TLC and LCMS monitoring), the reaction mixture was diluted with ice cold water (100 mL) and extracted with DCM (2×100 mL). The combined organic layer was washed with brine solution (2×50 mL), dried over Na₂SO₄, filtered and concentrated under reduced pressure. The crude was purified by flash chromatography (silica gel 100-200 M, 80 g SNAP) using 30-35% EtOAc in heptane to get the title compound as brown oil 90 (7 g, 89%).

LCMS=[M+H]⁺: 317.27; Purity=95%.

### Synthesis of tert-butyl(R)-(1-((2-acetylpyridin-4-yl)methyl)piperidin-3-yl)carbamate (91)

To a stirred solution of tert-butyl(R)-(1-((2-cyanopyridin-4-yl)methyl)piperidin-3-yl)carbamate 90 (5 g, 15.8 mmol) in THF (40 mL) was added MeMgBr (21.1 mL, 63.2 mmol; 3M in Et₂₀) dropwise at 0° C. The reaction mixture was stirred at RT for 5 h. After completion of reaction (TLC monitoring), the reaction mixture was cooled to 0° C., quenched with ice cold water (100 mL) and extracted with EtOAc (3×100 mL). The combined organic was washed with brine solution (2×50 mL), dried over Na₂SO₄ and evaporated under reduced pressure. The crude was purified by flash chromatography (silica gel 100-200 M, 40 g SNAP) using 25-30% EtOAc in heptane to get the title compound as a yellow solid 91 (1.8 g, 30%).

LCMS=[M+H]⁺: 334.32; Purity=94%.

### Synthesis of tert-butyl((3R)-1-((2-(1-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)ethyl)pyridin-4-yl)methyl)piperidin-3-yl)carbamate (92)

To a stirred solution of tert-butyl(R)-(1-((2-acetylpyridin-4-yl)methyl)piperidin-3-yl)carbamate 91 (1 g, 3 mmol) in MeOH (10 mL) were added 4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl) aniline 21 (1.76 g, 3.9 mmol) and acetic acid (0.3 mL, 6 mmol) sequentially at 0° C. and stirred at RT for 2 h. After that, NaCNBH₃ (359 mg, 6 mmol) was added to the reaction and stirred at 80° C. for 16 h. After completion of reaction (TLC monitoring), the reaction mixture was evaporated under reduced pressure. The crude was poured into ice cold water (20 mL) and extracted with EtOAc (3×50 mL). The combined organic layer was washed with brine solution (50 mL), dried over Na₂SO₄, filtered and evaporated under reduced pressure. The crude was purified by flash chromatography (silica gel 100-200 M, 12 g, SNAP) using 40-45% EtOAc in heptane to get the title compound as a light yellow solid 92 (750 mg, 21%).

LCMS=[M+H]⁺: 769.70; Purity=66%.

### Synthesis of (3R)-1-((2-(1-((4-(4-(3-oxa-8-azabicyclo[3.2.1]octan-8-yl)-7H-pyrrolo[2,3-d]pyrimidin-6-yl)phenyl)amino)ethyl)pyridin-4-yl)methyl)piperidin-3-amine (93)

To an ice cold stirred solution of tert-butyl((3R)-1-((2-(1-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)ethyl)pyridin-4-yl)methyl)piperidin-3-yl)carbamate 92 (750 mg, 0.644 mmol) in DCM (10 mL) was added TFA (5 mL) dropwise at 0° C. and stirred at RT for 2 h. After completion of reaction (TLC monitoring), the reaction mixture was concentrated under reduced pressure. The crude was triturated with Et₂O, decant and dried under reduced pressure. The crude was dissolved in 1,4-dioxane (5 mL), followed by 1,2-ethanediamine (86 μL, 1.29 mmol) was added at RT and stirred at 70° C. for 30 min. After completion of reaction (LCMS monitoring), the reaction mixture was poured into ice cold water (50 mL) and extracted with 25% IPA in CHCl₃ (3×50 mL). The combined organic layer was dried over Na₂SO₄, filtered and concentrated to get the title compound as a brown sticky solid 93 (260 mg; crude) which was used in next step without any purification.

LCMS=[M+H]⁺: 539.44; Purity=61%.

### Synthesis of (N-((3R)-1-((2-(1-((4-(4-(3-oxa-8-azabicyclo[3.2.1]octan-8-yl)-7H-pyrrolo[2,3-d]pyrimidin-6-yl)phenyl)amino)ethyl)pyridin-4-yl)methyl)piperidin-3-yl)acrylamide (Compound 625)

To a stirred solution of (3R)-1-((2-(1-((4-(4-(3-oxa-8-azabicyclo[3.2.1]octan-8-yl)-7H-pyrrolo[2,3-d]pyrimidin-6-yl)phenyl)amino)ethyl)pyridin-4-yl)methyl)piperidin-3-amine 93 (279 mg, 0.518 mmol) in THF (6 mL) was added K₃PO₄ (220 mg, 1.04 mmol) dissolved in water (3 mL) at 0° C. Then, 3-chloropropionyl chloride 17 (59.3 μL, 0.621 mmol) was added to the reaction mixture and stirred at RT for 1 h. Then, 2N aq. NaOH (3 mL, 6.21 mmol) was added dropwise at 0° C. and the reaction mixture was stirred at 60° C. for 1 h. After completion of reaction (TLC and LCMS monitoring), the reaction mixture was poured into ice cold water (50 mL) and extracted with 2-MeTHF (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get Compound 625 (60 mg, 19%). ¹H NMR (400 MHz, DMSO-d₆): δ 11.85 (s, 1H), 8.43 (t, J=4 Hz, 1H), 8.10 (s, 1H), 7.93-7.90 (m, 1H), 7.54 (d, J=7.6 Hz, 2H), 7.34 (d, J=3.2 Hz, 1H), 7.14 (dd, J=16 Hz, 4 Hz, 1H), 6.75 (brs, 1H), 6.56-6.51 (m, 2H), 6.45-6.37 (m, 1H), 6.25-6.15 (m, 1H), 6.07-6.02 (m, 1H), 5.54 (dd, J=9.6 Hz, 2.4 Hz, 1H), 4.84 (brs, 2H), 4.58-4.53 (m, 1H), 3.89-3.73 (m, 1H), 3.68-3.63 (m, 2H), 3.59-3.56 (m, 2H), 3.51-3.47 (m, 1H), 3.41-3.35 (m, 1H), 2.73-2.69 (m, 1H), 2.40-2.37 (m, 1H), 2.07-2.00 (m, 2H), 1.99-1.98 (m, 2H), 1.84-1.78 (m, 1H), 1.70-1.59 (m, 2H), 1.47-1.44 (m, 4H), 1.34-1.28 (m, 1H), 1.10-1.07 (m, 1H).

LCMS=[M+H]⁺: 593.39; Purity=97.33%.

### Example 23

### Compound 626 and Compound 627

Chiral separation of Compound 314 resulted in both enantiomers (R)-1-(4-(2,2,2-trifluoro-1-((4-(4-morpholino-7H-pyrrolo[2,3-d]pyrimidin-6-yl)phenyl)amino)ethyl)piperidin-1-yl)prop-2-en-1-one (Compound 626) and(S)-1-(4-(2,2,2-trifluoro-1-((4-(4-morpholino-7H-pyrrolo[2,3-d]pyrimidin-6-yl)phenyl)amino)ethyl)piperidin-1-yl)prop-2-en-1-one (Compound 627).

Note: Chirality arbitrarily assigned

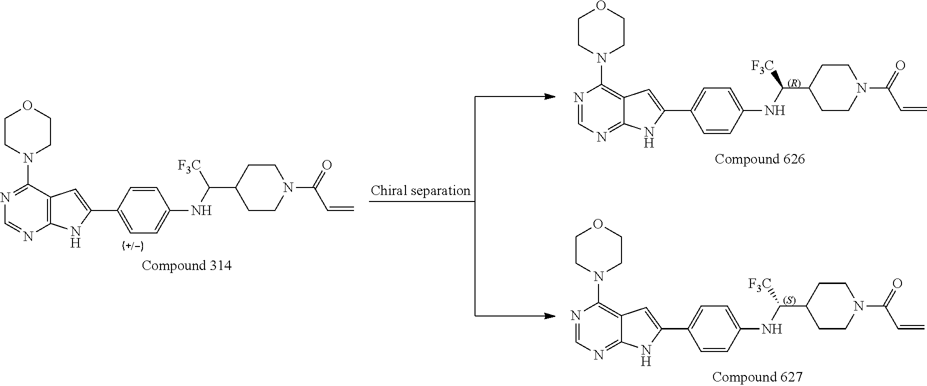

Methods for SFC purification: Column: Chiralcel OJ-H (250×30 mm),5u Mobile Phase: i) CO₂ ii) 0.2% TEA in MeOH; Flow rate: Isocratic 100 mL/Min, wavelength: 334 Analytical data of Compound 626:

¹H NMR (400 MHz, DMSO-d₆): δ 11.95 (s, 1H), 8.13 (s, 1H), 7.64 (d, J=8.8 Hz, 2H), 6.88-6.75 (m, 4H), 6.10-6.05 (m, 2H), 5.64-5.62 (m, 1H), 4.49-4.38 (m, 2H), 4.14-4.07 (m, 1H), 3.85-3.83 (m, 4H), 3.74-3.72 (m, 4H), 3.04 (q, J=12.4 Hz, 1H), 2.66-2.63 (m, 1H), 2.09 (m, 1H), 1.80-1.74 (m, 2H) and 1.42-1.31 (m, 2H).

LCMS: [M+H]⁺: 515.32; Purity=98.35%.

Analytical data of Compound 627:
¹H NMR (400 MHz, DMSO-d₆): δ 11.96 (s, 1H), 8.13 (s, 1H), 7.64 (d, J=8.8 Hz, 2H), 6.88-6.82 (m, 3H), 6.79-6.75 (m, 1H), 6.10-6.05 (m, 2H), 5.66-5.62 (m, 1H), 4.46-4.34 (m, 2H), 4.10-4.07 (m, 1H), 3.84 (m, 4H), 3.73 (m, 4H), 3.04 (q, J=12.4 Hz, 1H), 2.66-2.63 (m, 1H), 2.09 (m, 1H), 1.77-1.74 (m, 2H) and 1.42-1.38 (m, 2H). LCMS: [M+H]⁺: 515.34; Purity=97.45%.

### Example 24

### Compound 801

### Preparation of (R)-2-(1-((2-(((4-(4-morpholino-7H-pyrrolo[2,3-d]pyrimidin-6-yl)phenyl)amino)methyl)pyridin-4-yl)methyl)piperidin-3-yl) isothiazolidine 1,1-dioxide (Compound 801)

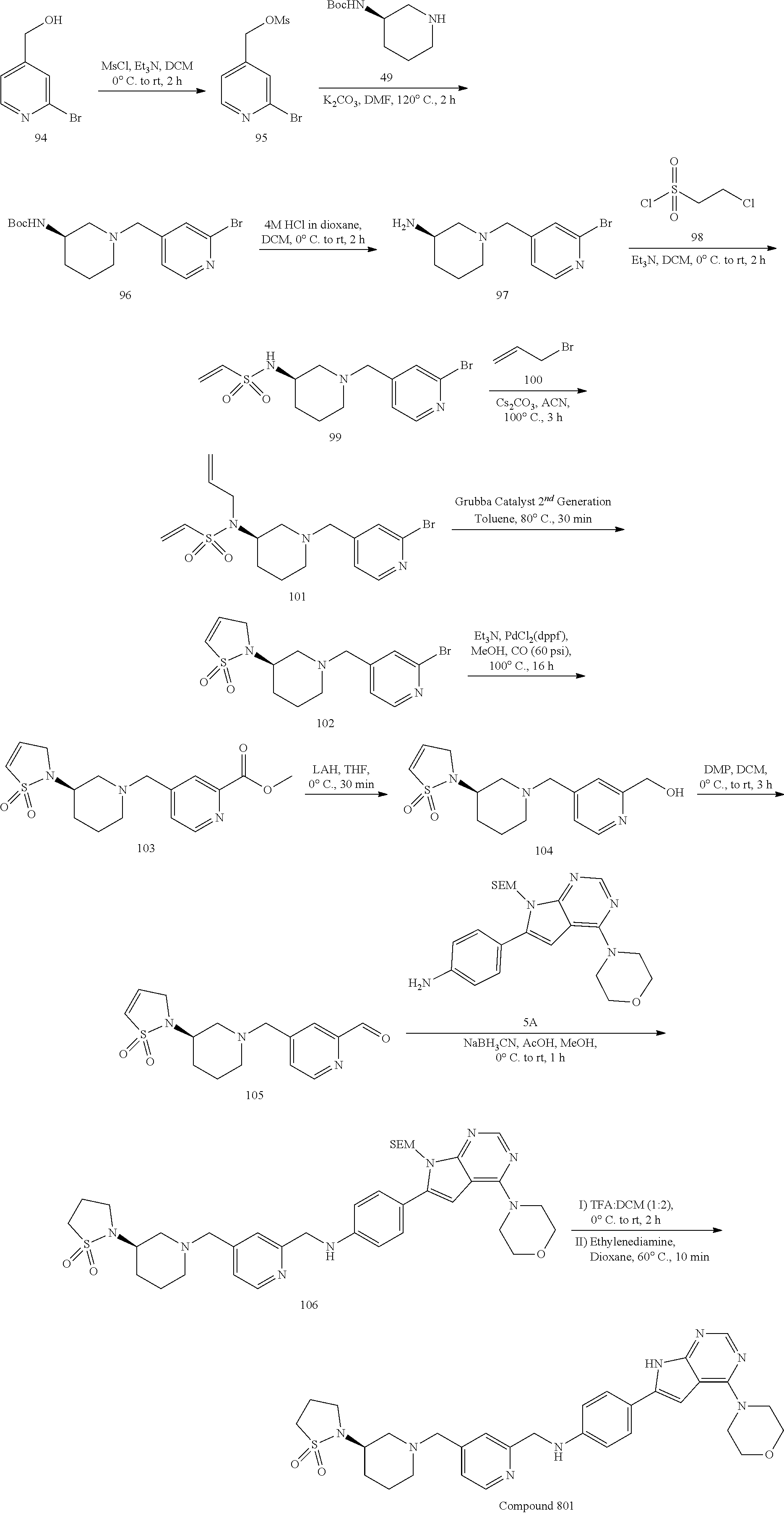

### Synthesis of (2-bromopyridin-4-yl)methyl methanesulfonate (95)

To a stirred solution of (2-bromopyridin-4-yl)methanol 94 (10 g, 53.2 mmol) in DCM (100 mL) were added TEA (18.4 mL, 133 mmol) and MsCl (6.17 mL, 79.8 mmol) at 0° C. under N₂ atmosphere. The resulting reaction mixture was stirred at RT for 2 h. After completion of reaction (TLC monitoring), the reaction mixture was diluted with DCM (100 mL) and washed with sat. NaHCO₃ (2×50 mL). The combined organic layer was dried over Na₂SO₄, filtered and concentrated under reduced pressure to get the title compound as a brown semi solid 95 (13 g; crude) which was used in next step directly without further purification.

LCMS=[M+H]⁺: 265.94; Purity=72%.

### Synthesis of tert-butyl(R)-(1-((2-bromopyridin-4-yl)methyl)piperidin-3-yl)carbamate (96)

To a stirred solution of (2-bromopyridin-4-yl)methyl methanesulfonate 95 (12 g, 45.1 mmol) and tert-butyl (R)-piperidin-3-ylcarbamate 49 (18.1 g, 90.2 mmol) in DMF (100 mL) was added K₂CO₃ (31.2 g, 225 mmol) at RT and stirred at 120° C. for 2 h. After completion of reaction (TLC monitoring), the reaction mixture was poured into ice cold water (200 mL) and extracted with EtOAc (3×100 mL). The combined organic layer was washed with ice cold brine solution (3×100 mL), dried over Na₂SO₄, filtered and evaporated under reduced pressure. The crude was purified by flash chromatography (silica gel 100-200 M, 120 g SNAP) using 20-30% EtOAc in heptane to get the title compound as a sticky liquid 96 (14.5 g, 86%) LCMS=[M+H]⁺: 370.18; Purity=99%.

### Synthesis of (R)-1-((2-bromopyridin-4-yl)methyl)piperidin-3-amine (97)

To a stirred solution of tert-butyl(R)-(1-((2-bromopyridin-4-yl)methyl)piperidin-3-yl)carbamate 96 (14.5 g, 39.2 mmol) in DCM (100 mL) was added 4M HCl in dioxane (50 mL) slowly at 0° C. and the reaction mixture was stirred at RT for 2 h. After completion of reaction (TLC monitoring), the reaction mixture was concentrated under reduced pressure. The crude was basified with sat. NaHCO₃ to pH~8-9 and extracted with 10% MeOH in DCM (3×100 mL). The combined organic layer was dried over Na₂SO₄, filtered and concentrated under reduced pressure to get the title compound as a brown semi solid 97 (8 g; crude) which was used in next step directly without further purification.

LCMS=[M+H]⁺: 270.17; Purity=85%.

### Synthesis of (R)—N-(1-((2-bromopyridin-4-yl)methyl)piperidin-3-yl) ethenesulfonamide (99)

To a stirred solution of (R)-1-((2-bromopyridin-4-yl)methyl)piperidin-3-amine 97 (11 g, 40.7 mmol) in DCM (2 mL) were added Et₃N(34.3 mL, 244 mmol) and 2-chloroethane-1-sulfonyl chloride 98 (4.71 mL, 44.8 mmol) dropwise at 0° C. and the reaction mixture was stirred at RT for 2 h. After completion of reaction (TLC monitoring), the reaction mixture was poured into ice cold water (100 mL) and extracted with DCM (3×100 mL). The combined organic layer was washed with brine (2×50 mL), dried over Na₂SO₄, filtered and concentrated under reduced pressure to get desired title compound as a brown semi solid 99 (12.5 g; crude) which was used in next step directly without further purification.

LCMS=[M+H]⁺: 360.15; Purity=65%.

### Synthesis of (R)—N-allyl-N-(1-((2-bromopyridin-4-yl)methyl)piperidin-3-yl) ethenesulfonamide (101)

To a stirred solution of (R)—N-(1-((2-bromopyridin-4-yl)methyl)piperidin-3-yl) ethenesulfonamide 99 (8 g, 22.2 mmol) in ACN(100 mL) were added Cs₂CO₃ (14.5 g, 44.4 mmol) and allyl bromide 100 (1.89 mL, 22.2 mmol) at 0° C. The resulting reaction mixture was stirred at 100° C. for 3 h. After completion of reaction (monitoring by TLC), the reaction mixture was diluted with water (100 mL) and extracted with EtOAc (3×100 mL). The combined organic layer was dried over Na₂SO₄, filtered and concentrated under reduced pressure. The crude compound was purified by flash chromatography (silica gel 100-200 M, 80 g SNAP) using 20-25% EtOAc in heptane to get the title compound as a sticky liquid 101 (6 g, 67%) LCMS=[M+H]⁺: 400.06; Purity=90%.

### Synthesis of (R)-2-(1-((2-bromopyridin-4-yl)methyl)piperidin-3-yl)-2,3-dihydroisothiazole 1,1-dioxide (102)

A solution of (R)—N-allyl-N-(1-((2-bromopyridin-4-yl)methyl)piperidin-3-yl) ethenesulfonamide 101 (6 g, 15 mmol) in toluene (600 mL) was purged with N₂ gas for 10 min. Then, Grubbs Catalyst 2nd Generation (6.36 g, 7.49 mmol) was added to the reaction and stirred at 80° C. for 30 min. After completion of reaction (monitoring by TLC), the reaction mixture was diluted with water (100 mL) and extracted with EtOAc (2×100 mL). The combined organic layer dried over Na₂SO₄, filtered and concentrated under reduced pressure. The crude was purified over the flash chromatography (silica gel 100-200 M, 40 g SNAP) using 60-80% EtOAc in heptane to get the title compound as a brown semi-solid 102 (3.8 g, 57%) LCMS=[M+H]⁺: 374.07; Purity=84%.

### Synthesis of methyl (R)-4-((3-(1,1-dioxidoisothiazol-2 (3H)-yl)piperidin-1-yl)methyl)picolinate (103)

To a stirred solution of (R)-2-(1-((2-bromopyridin-4-yl)methyl)piperidin-3-yl)-2,3-dihydroisothiazole 1,1-dioxide 102 (3.8 g, 10.2 mmol) in MeOH (50 mL) was added Et₃N(2.85 mL, 20.4 mmol) and the reaction mixture was degassed with N₂ gas for 10 minute. Then, PdCl₂ (dppf) (747 mg, 1.02 mmol) was added to the reaction mixture and stirred at 100° C. for 16 h under 60 psi pressure of CO gas. After completion of reaction (TLC and LCMS monitoring), the reaction mass was filtered through Celite pad and washed with MeOH. The filtrate was evaporated under reduced pressure. The crude was diluted with water (100 mL) and extracted with EtOAc (3×100 mL). The combined organic layer was dried over Na₂SO₄, filtered and concentrated under reduced pressure. The crude was purified by flash chromatography (silica gel 100-200 M, 40 g SNAP) using 2-5% MeOH in DCM to get the title compound as a brown sticky solid 103 (800 mg, 16%) LCMS=[M+H]⁺: 352.13; Purity=77%.

### Synthesis of (R)-2-(1-((2-(hydroxymethyl)pyridin-4-yl)methyl)piperidin-3-yl)-2,3-dihydroisothiazole 1,1-dioxide (104)

To a stirred solution of methyl (R)-4-((3-(1,1-dioxidoisothiazol-2 (3H)-yl)piperidin-1-yl)methyl)picolinate 103 (800 mg, 2.47 mmol) in THF (10 mL) was added LiAlH₄ (1.85 mL, 3.71 mmol; 2 M in THF) dropwise at 0° C. under N₂ atmosphere and stirred for 30 min at 0° C. After completion of reaction (TLC monitoring), the reaction mixture was quenched with sat. NH₄Cl (10 mL) and extracted with EtOAc (3×50 mL). The combined organic layer was washed with brine (2×50 mL), dried over Na₂SO₄, filtered and concentrated under reduced pressure to get the title compound as a light orange sticky solid 103 (450 mg; crude) which was used in next step directly without further purification.

LCMS=[M+H]⁺: 324.13; Purity=56%.

### Synthesis of (R)-4-((3-(1,1-dioxidoisothiazol-2 (3H)-yl)piperidin-1-yl)methyl)picolinaldehyde (105)

To a stirred solution of (R)-2-(1-((2-(hydroxymethyl)pyridin-4-yl)methyl)piperidin-3-yl)-2,3-dihydroisothiazole 1,1-dioxide (450 mg, 1.39 mmol) in DCM (10 mL) was added DMP (920 mg, 2.08 mmol) at 0° C. The reaction mixture stirred at RT for 3 h. After completion reaction (monitoring by TLC), the reaction mixture was quenched with sat. NaHCO₃ and extracted with DCM (3×50 mL). The combined organic layer was washed with brine (2×50 mL), dried over Na₂SO₄, filtered and concentrated under reduced pressure to get title compound as brown liquid 105 (460 mg; crude) which was used in next step directly without further purification.

### Synthesis of (R)-2-(1-((2-(((4-(4-morpholino-7-((2-(trimethylsilyl)ethoxy)methyl)-7H-pyrrolo[2,3-d]pyrimidin-6-yl)phenyl)amino)methyl)pyridin-4-yl)methyl)piperidin-3-yl) isothiazolidine 1,1-dioxide (106)

To a stirred solution of (R)-4-((3-(1,1-dioxidoisothiazol-2 (3H)-yl)piperidin-1-yl)methyl)picolinaldehyde 105 (453 mg, 1.41 mmol) in MeOH (10 mL) were added and 4-(4-morpholino-7-((2-(trimethylsilyl)ethoxy)methyl)-7H-pyrrolo[2,3-d]pyrimidin-6-yl) aniline 5A (400 mg, 0.940 mmol) and AcOH (cat) at 0° C. and stirred at RT for 20 min. Then, NaBH₃CN(112 mg, 1.88 mmol) was added to the reaction mixture at 0° C. and stirred at RT for 1 h. After completion of reaction (LCMS monitoring), the reaction mixture was quenched with sat. NaHCO₃ (50 mL) and extracted with DCM (3×50 mL). The combined organic layer was dried over Na₂SO₄, filtered and concentrated under reduce pressure. The crude was purified by flash chromatography (silica gel 100-200 M, 12 g SNAP) using 2-5% MeOH in DCM to get the title compound as a brown solid 106 (600 mg, 68%)

LCMS=[M+H]⁺: 733.65; Purity=78%.

### Synthesis of (R)-2-(1-((2-(((4-(4-morpholino-7H-pyrrolo[2,3-d]pyrimidin-6-yl)phenyl)amino)methyl)pyridin-4-yl)methyl)piperidin-3-yl) isothiazolidine 1,1-dioxide (Compound 801)

To a stirred solution of (R)-2-(1-((2-(((4-(4-morpholino-7-((2-(trimethylsilyl)ethoxy)methyl)-7H-pyrrolo[2,3-d]pyrimidin-6-yl)phenyl)amino)methyl)pyridin-4-yl)methyl)piperidin-3-yl) isothiazolidine 1,1-dioxide 106 (300 mg, 0.409 mmol) in DCM (2 mL) was added TFA (1 mL) dropwise at 0° C. and stirred at RT for 2 h. After completion of reaction (TLC and LCMS monitoring), the reaction mixture was concentrated under reduced pressure. The crude was dissolved in 1,4-dioxane (5 mL) and 1,2-ethanediamine (82 μL, 1.23 mmol) was added and stirred at 60° C. for 10 min. After completion of reaction (LCMS monitoring), the reaction mixture was diluted with water (50 mL) and extracted with 25% IPA in CHCl₃ (3×50 mL). The combined organic layer was dried over Na₂SO₄, filtered and concentrated under reduced pressure. The crude was purified by RP-HPLC [5-95% 5 mM Ammonium bicarbonate in Water/ACN; Column: X Select CSH C18 (19*250) 10μ] to get Compound 801 (30 mg, 12%).

¹H NMR (400 MHz, DMSO-d₆): δ 11.91 (s, 1H), 8.46 (d, J=5.2 Hz, 1H), 8.11 (s, 1H) 7.60 (d, J=8.8 Hz, 2H), 7.31 (s, 1H), 7.17 (d, J=4.4 Hz, 1H), 6.83 (d, J=1.6 Hz, 1H), 6.62 (d, J=8.8 Hz, 2H), 6.57 (t, J=6.4 Hz, 1H), 4.39 (d, J=6 Hz, 2H) 3.84-3.81 (m, 4H), 3.73-3.71 (m, 4H), 3.52-3.43 (m, 2H), 3.20-3.09 (m, 4H), 2.78 (d, J=16.4 Hz, 1H), 2.59-2.56 (m, 2H), 2.16-2.12 (m, 2H), 2.10-2.04 (m, 1H), 1.83-1.78 (m, 1H) 1.74-1.70 (m, 1H), 1.61-1.59 (m, 1H), 1.44-1.37 (m, 2H).

LCMS=[M+H]⁺: 603.33; Purity=99%.

### Example 25

### Compound 629

### Preparation of (R)—N-(1-((6-amino-5-((4-(4-morpholino-7H-pyrrolo[2,3-d]pyrimidin-6-yl)phenoxy)methyl)pyridin-3-yl)methyl)piperidin-3-yl)acrylamide (Compound 629)

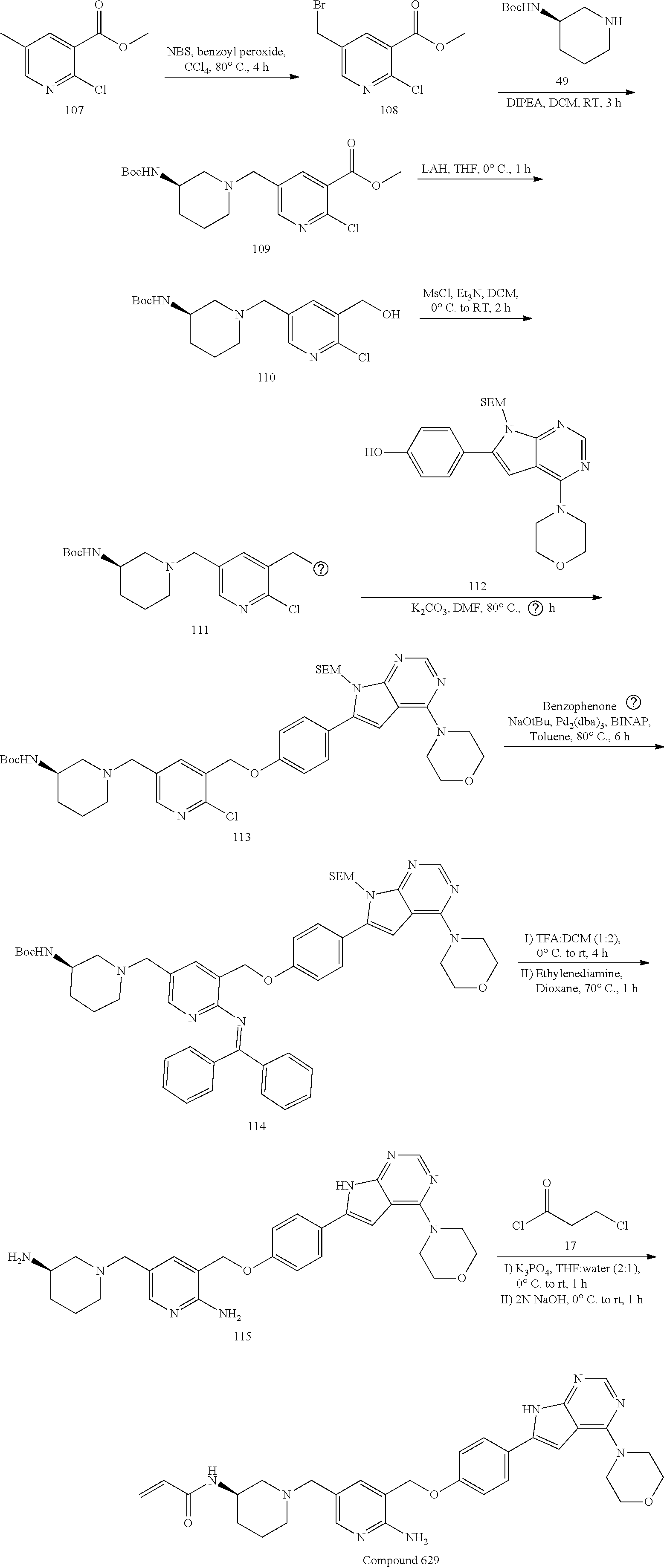

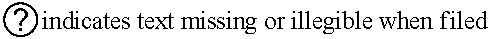

### Synthesis of methyl 5-(bromomethyl)-2-chloronicotinate (108)

To a stirred solution of methyl 2-chloro-5-methylnicotinate 107 (2.5 g, 13.5 mmol) in CCl₄ (25 mL) were added benzoyl peroxide (326 mg, 1.35 mmol) and NBS (2.16 g, 12.1 mmol) at RT under nitrogen atmosphere. The resulting reaction mixture was stirred at 80° C. for 4 h. After completion of reaction (TLC monitoring), the reaction mixture was concentrated under reduce pressure. The crude was purified flash chromatography (silica-gel 100-200 M, 40 g SNAP) using 7-10% EtOAc in heptane to get the title compound as yellow liquid 108 (1.8 g, 97%).

LCMS=[M+H]⁺: 265.92; Purity=77%.

### Synthesis of methyl (R)-5-((3-((tert-butoxycarbonyl)amino)piperidin-1-yl)methyl)-2-chloronicotinate (109)

To a stirred solution of methyl 5-(bromomethyl)-2-chloronicotinate 108 (2 g, 7.56 mmol) and (R)-3-piperidylamino-tert-butylformylate 49 (2.27 g, 11.3 mmol) in DCM (25 mL) was added DIPEA (3.91 mL, 22.7 mmol) at RT under nitrogen atmosphere and stirred for 3 h. After completion of reaction (TLC and LCMS monitoring), the resulting reaction mixture was diluted with water (30 mL) and extracted with DCM (3×30 mL). The combined organic layer was dried over Na₂SO₄, filtered and concentrated under reduced pressure. The crude was purified by flash chromatography (silica gel 100-200 M, 12 g SNAP) to get the title compound as a light yellow semi solid 109 (1.6 g, 55%).

LCMS=[M+H]⁺: 384.36; Purity=96%.

### Synthesis of tert-butyl(R)-(1-((6-chloro-5-(hydroxymethyl)pyridin-3-yl)methyl)piperidin-3-yl)carbamate (110)

To a stirred solution of methyl 5-({(R)-3-[(tert-butyl) (oxycarbonylamino)]-1-piperidyl}methyl)-2-chloronicotinate 109 (1.8 g, 4.69 mmol) in THF (50 mL) was added LiAlH₄ (2.8 mL, 5.63 mmol; 2M in THF) dropwise at −78° C. under nitrogen atmosphere and stirred for 2 h. After completion of reaction (TLC monitoring), the reaction mixture was quenched with ice cold water (2 mL), 15% aq. NaOH (2 mL), cold water (6 mL) at 0° C. and extracted with 25% IPA in CHCl₃ (3×50 mL). The combined organic layer was washed with brine (1×50 mL), dried over Na₂SO₄, filtered and concentrated under reduced pressure to get the title compound as a light yellow semi solid 110 (1.8 g; crude) which was used in next step directly without further purification.

LCMS=[M+H]⁺: 356.22; Purity=94%.

### Synthesis f (R)-(5-((3-((tert-butoxycarbonyl)amino)piperidin-1-yl)methyl)-2-chloropyridin-3-yl) methyl methanesulfonate (111)

To a stirred solution of (R)-1-{[6-chloro-5-(hydroxymethyl)-3-pyridyl]methyl}-3-piperidyl 2-methyl-2-propanecarbamate 110 (1 g, 2.81 mmol) in DCM (20 mL) were added Et₃N(1.17 mL, 8.43 mmol) and MsCl (0.32 mL, 4.22 mmol) at 0° C. The resulting reaction mixture was stirred at RT for 2 h. After completion of reaction (monitoring by TLC), the reaction mixture was quenched by water (50 mL) and extracted with DCM (3×50 mL). The combined organic layer was dried over Na₂SO₄, filtered and concentrated under reduced pressure to get the title compound as a pale yellow semi solid 111 (1.2 g; crude) which was used in next step directly without further purification.

LCMS=[M+H]⁺: 434.20; Purity=80%.

### Synthesis of tert-butyl(R)-(1-((6-chloro-5-((4-(4-morpholino-7-((2-(trimethylsilyl)ethoxy)methyl)-7H-pyrrolo[2,3-d]pyrimidin-6-yl)phenoxy)methyl)pyridin-3-yl)methyl)piperidin-3-yl)carbamate (113)

To a stirred solution of tert-butyl[(R)-1-({6-chloro-5-[(mesyloxy)methyl]-3-pyridyl}methyl)-3-piperidyl]carbamate 111 (1.57 g, 3.61 mmol) and p-(4-morpholino-1-{[2-(trimethylsilyl)ethoxy]methyl}-1H-1,5,7-triazainden-2-yl)phenol 112 (1.4 g, 3.28 mmol) in DMF (20 mL) was added K₂CO₃ (1.36 g, 9.85 mmol) at RT and the reaction mixture was stirred at 80° C. for 16 h. After completion of reaction of reaction (monitored by TLC and LCMS), the reaction mixture was cooled at RT, poured into ice cold water (50 mL) and extracted with EtOAc (3×50 mL). The combined organic layer was washed with brine (2×50 mL), and dried over Na₂SO₄, filtered and concentrated under reduced pressure. The crude was purified by flash chromatography (over neutral alumina) using 15-17% EtOAc in heptane to get the title compound as a light yellow solid 113 (1.4 g, 55%).

LCMS=[M+H]⁺: 764.64; Purity=71%.

### Synthesis of tert-butyl(R)-(1-((6-((diphenylmethylene)amino)-5-((4-(4-morpholino-7-((2-(trimethylsilyl)ethoxy)methyl)-7H-pyrrolo[2,3-d]pyrimidin-6-yl)phenoxy)methyl)pyridin-3-yl)methyl)piperidin-3-yl)carbamate (114)

To a stirred solution of tert-butyl[(R)-1-[(6-chloro-5-{[p-(4-morpholino-1-{[2-(trimethylsilyl)ethoxy]methyl}-1H-1,5,7-triazainden-2-yl)phenoxy]methyl}-3-pyridyl)methyl]-3-piperidyl]carbamate 113 (1.1 g, 1.44 mmol) and diphenylmethanimine (522 mg, 2.88 mmol) in toluene (15 mL) was added NaO′Bu (277 mg, 2.88 mmol) at RT. The resulting reaction mixture was degassed with argon gas for 15 min. After that, (+/−) BINAP (179 mg, 0.288 mmol) and Pd₂(dba)₃ (116 mg, 0.144 mmol) were added to the reaction and stirred at 80° C. for 6 h. After the completion of reaction (TLC monitoring), the reaction mixture was quenched with water (50 mL and extracted with EtOAc (3×50 mL). The combined organic layer was washed with brine (2×50 mL), dried over Na₂SO₄, filtered and evaporated under reduced pressure. The crude was purified by flash column chromatography (Neutral alumina, 40 g SNAP) using 15-20% EtOAc in heptane to get the title compound as a light yellow solid 114 (610 mg, 46%).

LCMS=[M+H]⁺: 909.80; Purity=83%.

### Synthesis (R)-5-((3-aminopiperidin-1-yl)methyl)-3-((4-(4-morpholino-7H-pyrrolo[2,3-d]pyrimidin-6-yl)phenoxy)methyl)pyridin-2-amine (115)

To a stirred solution of tert-butyl[(R)-1-{[6-(benzhydrylideneamino)-5-{[p-(4-morpholino-1-{[2-(trimethylsilyl)ethoxy]methyl}-1H-1,5,7-triazainden-2-yl)phenoxy]methyl}-3-pyridyl]methyl}-3-piperidyl]carbamate 114 (300 mg, 0.330 mmol) in DCM (6 mL) was added TFA (3 mL) dropwise at 0° C. and the reaction mixture was stirred at RT for 4 h. After the completion of reaction (TLC monitoring), the reaction mixture was evaporated under reduced pressure. The crude residue was triturated with Et₂₀ and dried over reduced pressure. Then the crude compound was dissolve in 1,4-dioxane (6 mL)) followed by, 1,2-ethanediamine (51.6 μL, 0.771 mmol) was added at RT and the reaction mixture was stirred at 70° C. for 1 h. After completion of reaction (LCMS monitoring), the reaction mixture was basified with sat. NaHCO₃ to PH~7-8 and extracted with 25% IPA in CHCl₃ (3×50 mL). The combined organic layer was dried over Na₂SO₄, filtered and evaporated under reduced pressure to get the title compound as a light yellow solid 115 (180 mg; crude) which was used in next step directly without further purification.

LCMS=[M+H]⁺: 515.46; Purity=73%

### Synthesis of (R)—N-(1-((6-amino-5-((4-(4-morpholino-7H-pyrrolo]2,3-d]pyrimidin-6-yl)phenoxy)methyl)pyridin-3-yl)methyl)piperidin-3-yl)acrylamide (Compound 629)

To a stirred solution of 5-{[(R)-3-amino-1-piperidyl]methyl}-3-{[p-(4-morpholino-1H-1,5,7-triazainden-2-yl)phenoxy]methyl}-2-pyridinamine 115 (210 mg, 0.408 mmol) in THF (4 mL) was added K₃PO₄ (173 mg, 0.816 mmol), dissolve in water (2 mL) at 0° C. Then, 3-chloropropionyl chloride 17 (62.2 mg, 0.490 mmol) dissolved in THF (1 mL) was added dropwise to the reaction mixture at 0° C. and stirred at RT for 1 h. After completion of SM (monitoring by TLC), 2N aq. NaOH (2.4 ml, 4.8 mmol) was added to the reaction mixture and stirred at RT for 1 h. After the completion of reaction (TLC and LCMS monitoring), the reaction mixture was diluted with water (20 mL) and extracted with 25% IPA in CHCl₃ (3×50 mL). The combined organic was dried over Na₂SO₄, filtered and concentrated under reduce pressure. The crude was purified by RP-HPLC [5-95% 5 mM Ammonium Bicarbonate in Water/ACN; Column: X Select Phenyl Hexyl (19*250) 5u)] to get Compound 629 (32 mg, 13%).

¹H NMR (400 MHz, DMSO-d₆): δ 12.14 (s, 1H), 8.17 (s, 1H), 7.94 (d, J=8.4 Hz, 1H), 7.86 (d, J=8.8 Hz, 2H), 7.81 (d, J=2.4 Hz, 1H), 7.48 (d, J=2 Hz, 1H), 7.11 (d, J=8.8 Hz, 2H), 7.06 (d, J=2 Hz, 1H), 6.25-6.18 (m, 1H), 6.05 (dd, J=17.2 Hz, 2.4 Hz, 1H), 5.85 (s, 2H), 5.54 (dd, J=10 Hz, 2.4 Hz, 1H), 4.99 (s, 2H), 3.88-3.86 (m, 4H), 3.76-3.73 (m, 5H), 2.73-2.71 (m, 1H), 2.61-2.58 (m, 3H), 1.90-1.79 (m, 1H), 1.77-1.69 (m, 2H), 1.65-1.61 (m, 1H), 1.45-1.41 (m, 1H), 1.19-1.12 (m, 1H),

LCMS=[M+H]: 569.39; Purity=99.36%.

### Example 26

### Compound 704 and Compound 705

Chiral separation of B028-212 resulted in both enantiomers (R)—N,N-dimethyl-4-(4-(2,2,2-trifluoro-1-((4-(4-morpholino-7H-pyrrolo[2,3-d]pyrimidin-6-yl)phenyl)amino)ethyl)piperidin-1-yl)but-2-ynamide (Compound 704) and(S)—N,N-dimethyl-4-(4-(2,2,2-trifluoro-1-((4-(4-morpholino-7H-pyrrolo[2,3-d]pyrimidin-6-yl)phenyl)amino)ethyl)piperidin-1-yl)but-2-ynamide (Compound 705)

Note: Chirality arbitrarily assigned

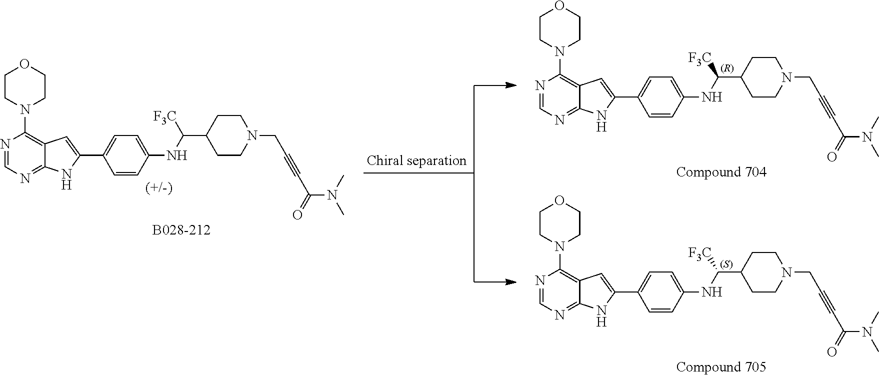

Methods for SFC purification: Column: Chiralpak AS-H (21×250 mm), 5u Mobile Phase: i) CO₂ ii) 0.2% TEA in MeOH; Flow rate: Isocratic 70 mL/Min, wavelength: 335

Analytical data of Compound 704:

¹H NMR (400 MHz, DMSO-d₆): δ 11.96 (s, 1H), 8.14 (s, 1H), 7.65 (d, J=8.8 Hz, 2H), 6.89-6.84 (m, 3H), 6.09 (d, J=10 Hz, 1H), 4.32-4.26 (m, 1H), 3.86-3.84 (m, 4H), 3.75-3.73 (m, 4H), 3.49 (s, 2H), 3.14 (s, 3H), 2.86-2.81 (m, 5H), 2.17-2.11 (m, 2H), 1.76-1.70 (m, 3H), 1.52-1.45 (m, 2H).

LCMS=[M+H]⁺: 570.37; Purity=99.88%.

Analytical data of Compound 705:

¹H NMR (400 MHz, DMSO-d₆): δ 11.97 (s, 1H), 8.14 (s, 1H), 7.65 (d, J=8.8 Hz, 2H), 6.89-6.84 (m, 3H), 6.09 (d, J=10 Hz, 1H), 4.30-4.28 (m, 1H), 3.86-3.84 (m, 4H), 3.75-3.73 (m, 4H), 3.49 (s, 2H), 3.14 (s, 3H), 2.86-2.81 (m, 5H), 2.17-2.11 (m, 2H), 1.73-1.70 (m, 3H), 1.52-1.46 (m, 2H).

LCMS=[M+H]⁺: 570.40; Purity=99.78%.

### Example 27

### Compound 706

Preparation of N,N-dimethyl-4-(3-(4-(4-morpholino-7H-pyrrolo[2,3-d]pyrimidin-6-yl)phenyl)-2,4-dioxo-1,3,7-triazaspiro[4.4]nonan-7-yl)but-2-ynamide (Compound 706)

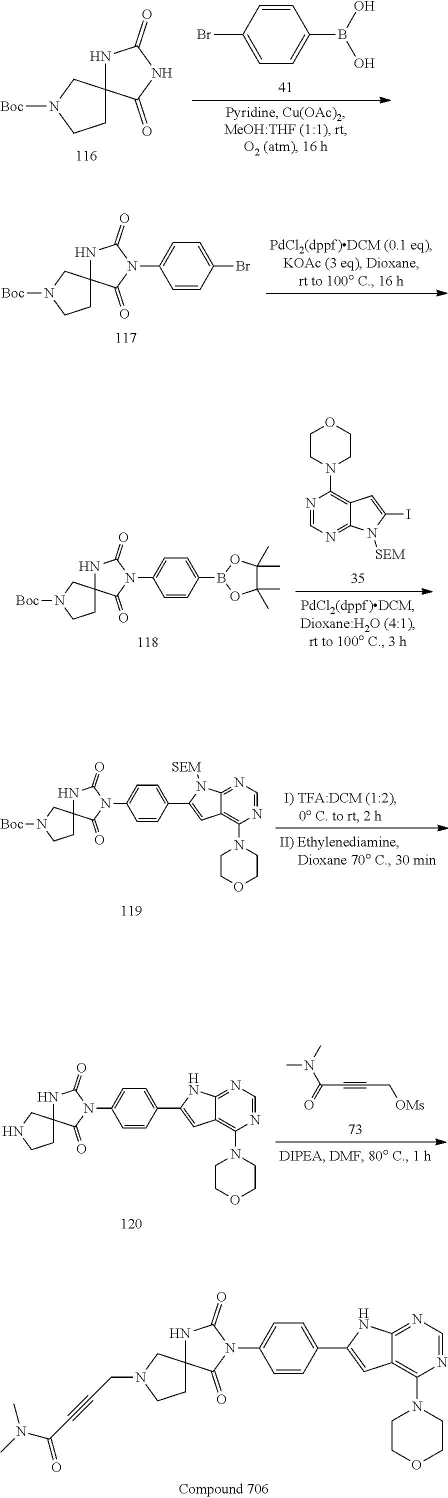

### Synthesis of tert-butyl 3-(4-bromophenyl)-2,4-dioxo-1,3,7-triazaspiro[4.4]nonane-7-carboxylate (117)

To a stirred solution of tert-butyl 2,4-dioxo-1,3,7-triaza-7-spiro[4.4]nonanecarboxylate 116 (2 g, 7.83 mmol) and (4-bromophenyl) boronic acid 41 (2.36 g, 1.5 eq., 11.8 mmol) in MeOH (20 mL) and THF (20 mL) was added pyridine (1.9 mL, 3 eq., 23.5 mmol) was added at RT. The reaction mixture was purged with O₂ gas for 15 min. After that, Cu(OAc) 2 (2.13 g, 11.8 mmol) was added to the reaction mixture and stirred at RT for 16 h under O₂ atmosphere. After the completion of reaction (LCMS monitoring), the reaction mixture was filter through the Celite pad and washed with EtOAc. The filtrate was diluted with EtOAc (100 mL) and washed with water (3×50 mL) and brine (2×50 mL). The organic layer was dried over Na₂SO₄, filtered and evaporated under reduced pressure. The crude was purified by flash chromatography (silica gel 100-200 M, 40 g SNAP) using 45-50% EtOAc in heptane to get the title compound as a white solid 117 (1.5 g, 46%).

LCMS=[M+H]⁺: 410.1; Purity=98.45%.

### Synthesis of tert-butyl 2,4-dioxo-3-(4-(4,4,5,5-tetramethyl-1,3,2-dioxaborolan-2-yl)phenyl)-1,3,7-triazaspiro[4.4]nonane-7-carboxylate (118)

To a stirred solution of tert-butyl 3-(p-bromophenyl)-2,4-dioxo-1,3,7-triaza-7-spiro[4.4]nonanecarboxylate 117 (1.5 g. 3.66 mmol) in 1,4-dioxane (20 mL) were added B₂Pin₂ (1.11 g, 4.39 mmol) and KOAc (1.08 g, 11 mmol). The reaction mixture was degassed with N₂ gas for 15 min. Then, PdCl₂(dppf).DCM (298 mg, 0.366 mmol) was added and the reaction mixture was stirred at 100° C. for 16 h. After completion of reaction (monitoring by TLC), the reaction mixture was filtered through Celite pad and washed with EtOAc. The filtrate was concentrated under reduced pressure to get the title compound as brown sticky solid 118 (1.3 g; crude) which was used in next step without further purification.

### Synthesis of tert-butyl 3-(4-(4-morpholino-7-((2-(trimethylsilyl)ethoxy)methyl)-7H-pyrrolo[2,3-d]pyrimidin-6-yl)phenyl)-2,4-dioxo-1,3,7-triazaspiro[4.4]nonane-7-carboxylate (119)

To a stirred solution of tert-butyl 2,4-dioxo-3-[p-(4,4,5,5-tetramethyl-1,3,2-dioxaborolan-2-yl)phenyl]-1,3,7-triaza-7-spiro[4.4]nonanecarboxylate 119 (1.3 g, 2.84 mmol) in 1,4-dioxane (24 mL) and water (6 mL) were added K₂CO₃ (842 mg, 6.09 mmol) and {2-[(2-iodo-4-morpholino-1H-1,5,7-triazainden-1-yl)methoxy]ethyl}tris(methyl) silane 35 (935 mg, 2.03 mmol) at RT. The reaction mixture was degassed with N₂ gas for 15 min. Then, PdCl₂(dppf).DCM (149 mg, 0.203 mmol) was added to the reaction mixture and stirred at 100° C. for 3 h. After completion of reaction (TLC and LCMS monitoring), the reaction mixture was diluted with EtOAc (100 mL) and washed with water (2×50 mL). The organic layer was dried over Na₂SO₄, filtered and evaporated under reduced pressure. The crude was purified by flash chromatography (silica gel 100-200 M, 40 g SNAP) using 30-40% EtOAc in heptane to get the title compound as a brown solid 119 (1.3 g, 96%). LCMS=[M+H]⁺: 664.85; Purity=82%.

### Synthesis of 3-(4-(4-morpholino-7H-pyrrolo[2,3-d]pyrimidin-6-yl)phenyl)-1,3,7-triazaspiro[4.4]nonane-2,4-dione (120)

To a stirred solution of tert-butyl 3-[p-(4-morpholino-1-{[2-(trimethylsilyl)ethoxy]methyl}-1H-1,5,7-triazainden-2-yl)phenyl]-2,4-dioxo-1,3,7-triaza-7-spiro[4.4]nonanecarboxylate 119 (1.3 g, 1.96 mmol) in DCM (20 mL) was added TFA (10 mL) dropwise at 0° C. and the reaction mixture was stirred at RT for 2 h. After completion of reaction (TLC and LCMS monitoring), the reaction mixture was concentrated under reduced pressure, triturated with diethyl ether and dried under reduced pressure. The crude was dissolved in 1,4-dioxane (10 mL), followed by 1,2-ethanediamine (0.26 mL, 3.92 mmol) was added at RT and stirred at 70° C. for 30 min. After completion of reaction (monitoring by LCMS), the reaction was poured into ice cold water (20 mL) and extracted with 25% IPA in CHCl₃ (3×50 mL). The combined organic layer was dried over Na₂SO₄, filtered and concentrated under reduced pressure to get the title compound as a yellow solid 120 (800 mg; crude) which was used in next step without further purification.

LCMS=[M+H]⁺: 434.47; Purity=84%.

### Synthesis of N,N-dimethyl-4-(3-(4-(4-morpholino-7H-pyrrolo[2,3-d]pyrimidin-6-yl)phenyl)-2,4-dioxo-1,3,7-triazaspiro[4.4]nonan-7-yl)but-2-ynamide (Compound 706)

To a stirred solution of 3-[p-(4-morpholino-1H-1,5,7-triazainden-2-yl)phenyl]-1,3,7-triaza-2,4-spiro[4.4]nonanedione 120 (400 mg, 0.923 mmol) in DMF (6 mL) were added N,N-dimethyl-4-(mesyloxy)-2-butynamide 73 (227 mg, 1.11 mmol) and Et₃N(0.39 mL, 2.77 mmol) at RT and the reaction mixture was stirred for 15 min at 80° C. After completion of reaction (monitoring by TLC), the reaction mixture was poured into ice cold water (50 mL) and extracted with EtOAc (3×50 mL). The combined organic layer was washed with ice cold brine (2×50 mL), dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get Compound 706 (31 mg, 6%).

¹H NMR (400 MHz, DMSO-d₆): δ 12.29 (s, 1H), 9.01 (s, 1H), 8.19 (s, 1H), 7.99 (d, J=8.4 Hz, 2H), 7.44 (d, J=8.8 Hz, 2H), 7.25 (d, J=1.6 Hz, 1H), 3.91-3.88 (m, 4H), 3.76-3.74 (m, 4H), 3.71 (s, 2H), 3.17 (s, 3H), 2.96-2.95 (m, 3H), 2.85 (s, 3H), 2.65-2.64 (m, 1H), 2.41-2.33 (m, 1H), 2.06-1.99 (m, 1H).

LCMS=[M+H]⁺: 543.60; Purity=98.90%.

### Example 28

### Compound 611

### Preparation of (R)-4-((3-(2-((methylsulfonyl)methyl)acrylamido)piperidin-1-yl)methyl)-N-(4-(4-morpholino-7H-pyrrolo[2,3-d]pyrimidin-6-yl)phenyl)picolinamide (Compound 611)

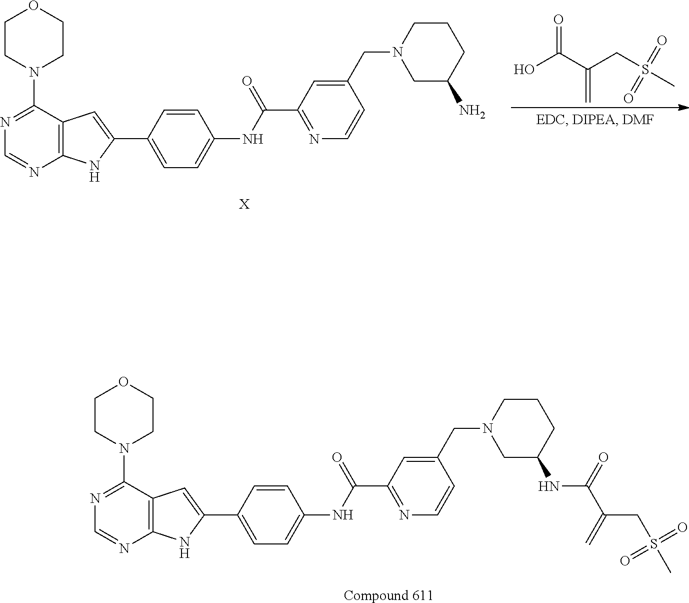

(R)-4-((3-aminopiperidin-1-yl)methyl)-N-(4-(4-morpholino-7H-pyrrolo[2,3-d]pyrimidin-6-yl)phenyl)picolinamide trihydrochloride X (82 mg, 0.132 mmol) was added to a solution of 1-(3-dimethylaminopropyl)-3-ethylcarbodiimide hydrochloride (33 mg, 0.171 mmol), 2-(methylsulfonylmethyl)prop-2-enoic acid (28 mg, 0.171 mmol), and N,N-diisopropylethylamine (0.18 mL, 1.05 mmol) in N,N-dimethylformamide (5 mL). After 18 hours the reaction was subjected to preparative HPLC(5-85% acetonitrile/water, 0.1% trifluoroacetic acid). The fractions containing product were combined and subjected to lyophilization, providing Compound 611 (11 mg, 0.0134 mmol, 10%). ¹H NMR (400 MHz, MeOD) δ 8.74 (t, J=5.1 Hz, 1H), 8.29 (d, J=1.7 Hz, 1H), 8.20 (s, 1H), 7.91-7.83 (m, 2H), 7.81-7.73 (m, 2H), 7.64 (m, 1H), 7.18 (s, 1H), 6.00 (s, 1H), 5.78 (s, 1H), 4.43 (s, 2H), 4.14-4.03 (m, 2H), 3.98 (t, J=4.9 Hz, 4H), 3.83 (dd, J=5.8, 4.0 Hz, 5H), 3.43 (d, J=13.8 Hz, 2H), 2.98 (m, 2H), 2.88 (m, 3H), 2.07-1.88 (m, 2H), 1.80 (m, 1H), 1.59 (s, 1H).

LCMS=[M+H]⁺: 659.30; Purity=95%.

### Example 29

### Compound 612

### Preparation of (R)-4-((3-(4-(dimethylamino)-2-methylenebutanamido)piperidin-1-yl)methyl)-N-(4-(4-morpholino-7H-pyrrolo[2,3-d]pyrimidin-6-yl)phenyl)picolinamide (Compound 612)

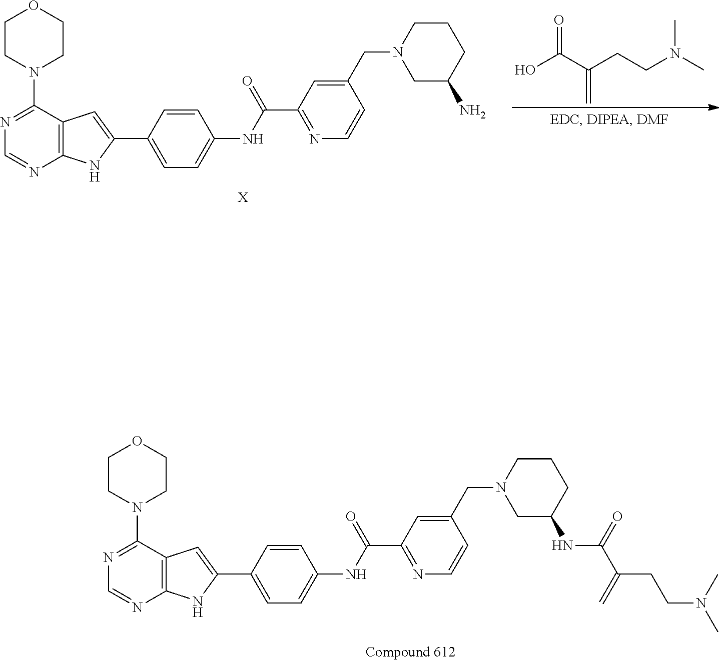

(R)-4-((3-aminopiperidin-1-yl)methyl)-N-(4-(4-morpholino-7H-pyrrolo[2,3-d]pyrimidin-6-yl)phenyl)picolinamide trihydrochloride X (75 mg, 0.121 mmol) was added to a solution of 1-(3-dimethylaminopropyl)-3-ethylcarbodiimide hydrochloride (35 mg, 0.181 mmol), 4-(dimethylamino)-2-methylene-butanoic acid (28 mg, 0.157 mmol), and N,N-diisopropylethylamine (0.17 mL, 0.965 mmol) in N,N-dimethylformamide (5 mL). After 20 hours the reaction was subjected to preparative HPLC(5-85% acetonitrile/water, 0.1% trifluoroacetic acid). The fractions containing product were combined and subjected to lyophilization, providing Compound 612 (65 mg, 0.0813 mmol, 67%). ¹H NMR (400 MHz, MeOD) & 8.87 (d, J=4.9 Hz, 1H), 8.42 (s, 1H), 8.33 (s, 1H), 8.04-7.96 (m, 2H), 7.95-7.87 (m, 2H), 7.80 (dd, J=4.9, 1.8 Hz, 1H), 7.32 (s, 1H), 5.93 (s, 1H), 5.71 (s, 1H), 4.54 (s, 2H), 4.23 (m, 1H), 4.11 (t, J=4.9 Hz, 4H), 3.95 (t, J=4.9 Hz, 4H), 3.55 (m, 1H), 3.47 (m, 1H), 3.27 (dd, J=8.9, 6.1 Hz, 2H), 3.12 (m, 2H), 2.72 (t, J=7.5 Hz, 2H), 2.18-1.99 (m, 2H), 1.99-1.83 (m, 1H), 1.70 (m, 1H).

LCMS=[M+H]⁺: 638.40; Purity=95%.

### Example 30

### Compound 613

### Preparation of (R)-4-((3-(2-(3,3-difluoroazetidin-1-yl)acrylamido)piperidin-1-yl)methyl)-N-(4-(4-morpholino-7H-pyrrolo[2,3-d]pyrimidin-6-yl)phenyl)picolinamide (Compound 613)

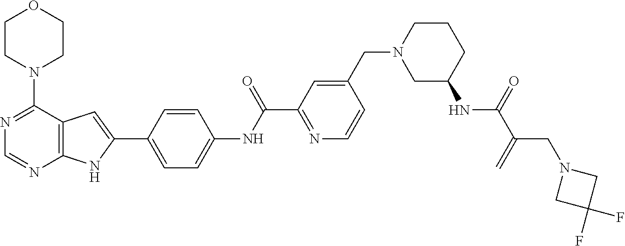 [SMILES: C=C(CN1CC(F)(F)C1)C(=O)N[C@@H]1CCCN(Cc2ccnc(C(=O)Nc3ccc(-c4cc5c(N6CCOCC6)ncnc5[nH]4)cc3)c2)C1]

Step 1:2-((3,3-Difluoroazetidin-1-yl)methyl)acrylic acid

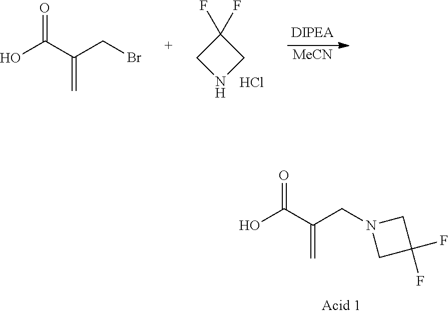

A solution of 2-(bromomethyl)prop-2-enoic acid (250 mg, 1.52 mmol), 3,3-difluoroazetidine hydrochloride (255 mg, 1.97 mmol), and N,N-diisopropylethylamine (0.79 mL, 4.55 mmol) in acetonitrile (4 mL) was stirred at 35° C. for 20 hours, providing a solution of 2-((3,3-difluoroazetidin-1-yl)methyl)acrylic acid (266 mg, 1.50 mmol, 99%) in acetonitrile. The reaction was assumed to be quantitative. Aliquots of this solution were used in the next step without workup.

Step 2: (R)-4-((3-(2-(3,3-difluoroazetidin-1-yl)acrylamido)piperidin-1-yl)methyl)-N-(4-(4-morpholino-7H-pyrrolo[2,3-d]pyrimidin-6-yl)phenyl)picolinamide (Compound 613)

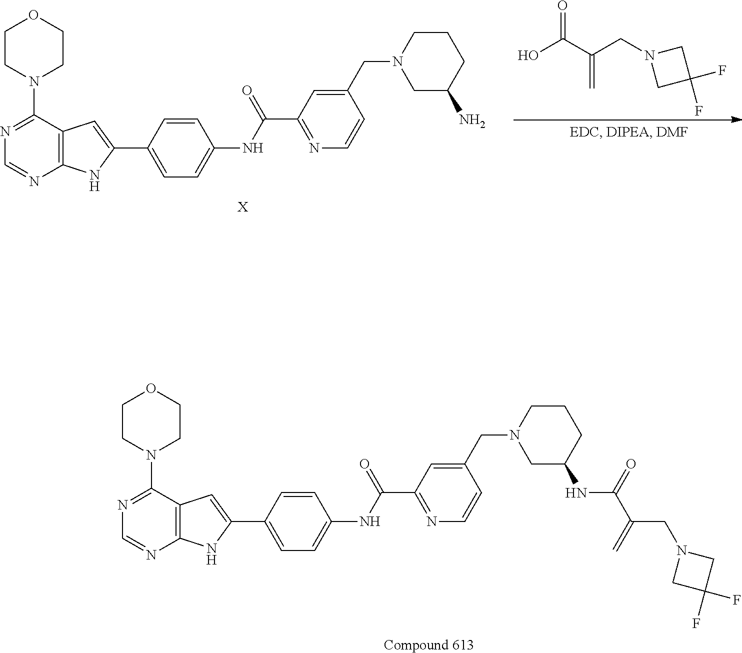

(R)-4-((3-aminopiperidin-1-yl)methyl)-N-(4-(4-morpholino-7H-pyrrolo[2,3-d]pyrimidin-6-yl)phenyl)picolinamide trihydrochloride X (75 mg, 0.121 mmol) was added to a solution of 1-(3-dimethylaminopropyl)-3-ethylcarbodiimide hydrochloride (35 mg, 0.181 mmol), 2-((3,3-difluoroazetidin-1-yl)methyl)acrylic acid (32 mg, 0.181 mmol) (as a solution in acetonitrile), and N,N-diisopropylethylamine (0.17 mL, 0.965 mmol) in N,N-dimethylformamide (5 mL). After 27 hours the mixture was subjected to preparative HPLC(5-85% acetonitrile/water, 0.1% trifluoroacetic acid). The fractions containing product were combined and subjected to lyophilization, providing of Compound 613 (66 mg, 0.0796 mmol, 65%). ¹H NMR (400 MHz, MeOD) & 8.75 (d, J=4.9 Hz, 1H), 8.33-8.27 (m, 1H), 8.22 (s, 1H), 7.92-7.84 (m, 2H), 7.83-7.75 (m, 2H), 7.67 (dd, J=4.9, 1.8 Hz, 1H), 7.21 (s, 1H), 6.11 (s, 1H), 5.89 (s, 1H), 4.49-4.33 (m, 6H), 4.11 (d, J=10.3 Hz, 1H), 4.00 (t, J=4.9 Hz, 4H), 3.93 (s, 2H), 3.89-3.78 (m, 4H), 3.44 (m, 1H), 3.38 (s, 1H), 2.96 (m, 2H), 2.09-1.89 (m, 2H), 1.89-1.72 (m, 1H), 1.60 (m, 1H).

LCMS=[M+H]⁺: 672.40; Purity=95%.

### Example 31

### Compound 614

### Preparation of (R)-4-((3-(2-((3-Fluoro-3-methylazetidin-1-yl)methyl)acrylamido)piperidin-1-yl)methyl)-N-(4-(4-morpholino-7H-pyrrolo[2,3-d]pyrimidin-6-yl)phenyl)picolinamide (Compound 614)

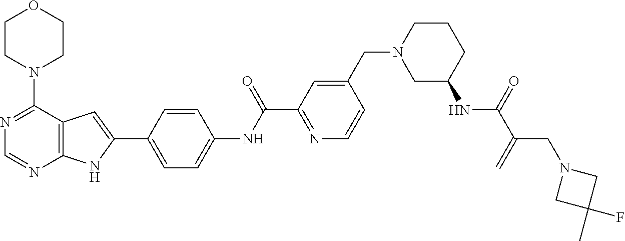 [SMILES: C=C(CN1CC(C)(F)C1)C(=O)N[C@@H]1CCCN(Cc2ccnc(C(=O)Nc3ccc(-c4cc5c(N6CCOCC6)ncnc5[nH]4)cc3)c2)C1]

Step 1:2-((3-fluoro-3-methylazetidin-1-yl)methyl)acrylic acid

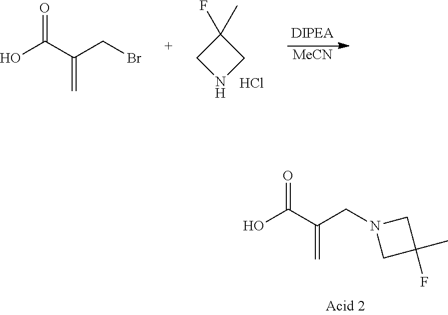

A solution of 2-(bromomethyl)prop-2-enoic acid (125 mg, 0.758 mmol), 3-fluoro-3-methyl-azetidine hydrochloride (124 mg, 0.985 mmol), and N,N-diisopropylethylamine (0.40 mL, 2.27 mmol) in acetonitrile (4 mL) was stirred at 35° C. for 1 hour, providing a solution of 2-((3-fluoro-3-methylazetidin-1-yl)methyl)acrylic acid (131 mg, 0.756 mmol, 99%) in acetonitrile. The reaction was assumed to be quantitative. Aliquots of this solution were used in the next step without workup.

LCMS=[M−H]—: 172.20; Purity=95%.

Step 2: (R)-4-((3-(2-((3-Fluoro-3-methylazetidin-1-yl)methyl)acrylamido)piperidin-1-yl)methyl)-N-(4-(4-morpholino-7H-pyrrolo[2,3-d]pyrimidin-6-yl)phenyl)picolinamide (Compound 614)

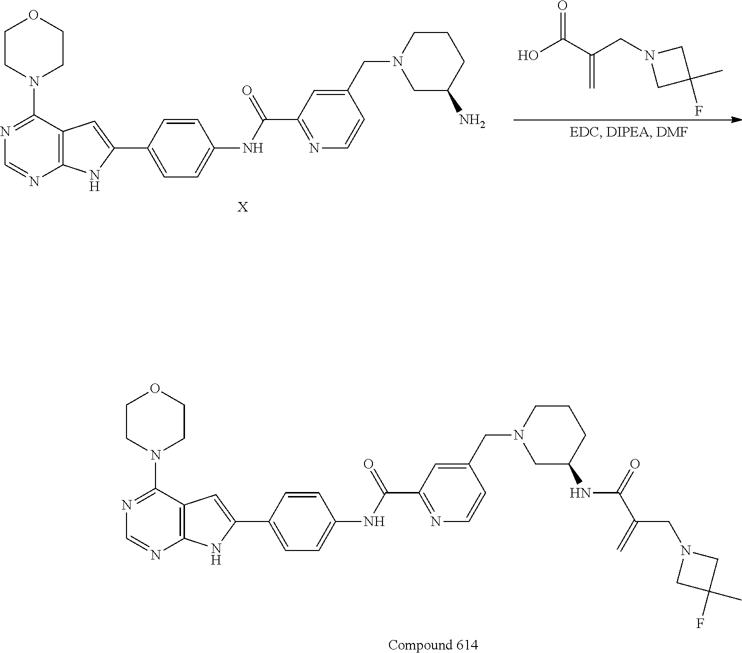

(R)-4-((3-aminopiperidin-1-yl)methyl)-N-(4-(4-morpholino-7H-pyrrolo[2,3-d]pyrimidin-6-yl)phenyl)picolinamide trihydrochloride X (75 mg, 0.121 mmol) was added to a solution of 1-(3-dimethylaminopropyl)-3-ethylcarbodiimide hydrochloride (35 mg, 0.181 mmol), 2-((3-fluoro-3-methylazetidin-1-yl)methyl)acrylic acid (31 mg, 0.181 mmol) (as a solution in acetonitrile) and N,N-diisopropylethylamine (0.17 mL, 0.965 mmol) in N,N-dimethylformamide (5 mL). After 18 hours the reaction was subjected to preparative HPLC(5-85% acetonitrile/water, 0.1% trifluoroacetic acid). The fractions containing product were combined and subjected to lyophilization, providing Compound 614 (30 mg, 0.0360 mmol 29%). ¹H NMR (400 MHz, MeOD) δ 8.75 (d, J=4.9 Hz, 1H), 8.30 (d, J=1.7 Hz, 1H), 8.21 (s, 1H), 7.92-7.84 (m, 2H), 7.84-7.76 (m, 2H), 7.67 (dd, J=5.0, 1.7 Hz, 1H), 7.19 (s, 1H), 6.17 (s, 1H), 5.96 (s, 1H), 4.42 (s, 2H), 4.24 (m, 4H), 4.12 (m, 1H), 4.05-3.90 (m, 6H), 3.83 (dd, J=5.8, 4.0 Hz, 4H), 3.42 (m, 1H), 3.35 (m, 1H), 2.97 (m, 2H), 1.97 (m, 2H), 1.88-1.70 (m, 1H), 1.60 (s, 1H), 1.54 (d, J=21.9 Hz, 3H) LCMS=[M+H]⁺: 668.40; Purity=95%.

### Example 32

### Compound 615

### Preparation of (R)-4-((3-(2-((tert-butyl(methyl)amino)methyl)acrylamido)piperidin-1-yl)methyl)-N-(4-(4-morpholino-7H-pyrrolo[2,3-d]pyrimidin-6-yl)phenyl)picolinamide (Compound 615)

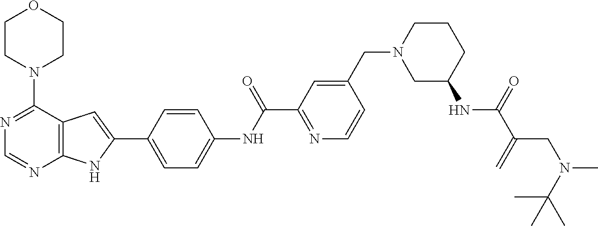 [SMILES: C=C(CN(C)C(C)(C)C)C(=O)N[C@@H]1CCCN(Cc2ccnc(C(=O)Nc3ccc(-c4cc5c(N6CCOCC6)ncnc5[nH]4)cc3)c2)C1]

Step 1:2-((tert-butyl(methyl)amino)methyl)acrylic acid

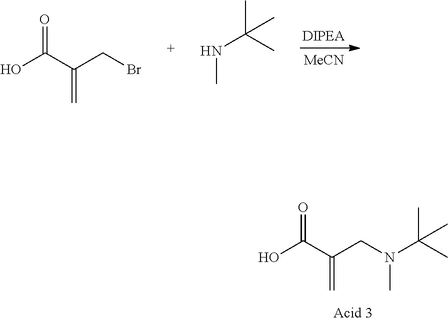

A solution of 2-(bromomethyl)prop-2-enoic acid (250 mg, 1.52 mmol)N-methyl-tert-butylamine (0.20 mL, 1.67 mmol), and N,N-diisopropylethylamine (0.79 mL, 4.55 mmol) in acetonitrile (4 mL) was stirred at 35° C. for 20 hours, providing a solution of 2-((tert-butyl(methyl)amino)methyl)acrylic acid (259 mg, 1.51 mmol, 99%) in acetonitrile. The reaction was assumed to be quantitative. Aliquots of this solution were used in the next step without workup.

Step 2: (R)-4-((3-(2-((tert-butyl(methyl)amino)methyl)acrylamido)piperidin-1-yl)methyl)-N-(4-(4-morpholino-7H-pyrrolo[2,3-d]pyrimidin-6-yl)phenyl)picolinamide (Compound 615)

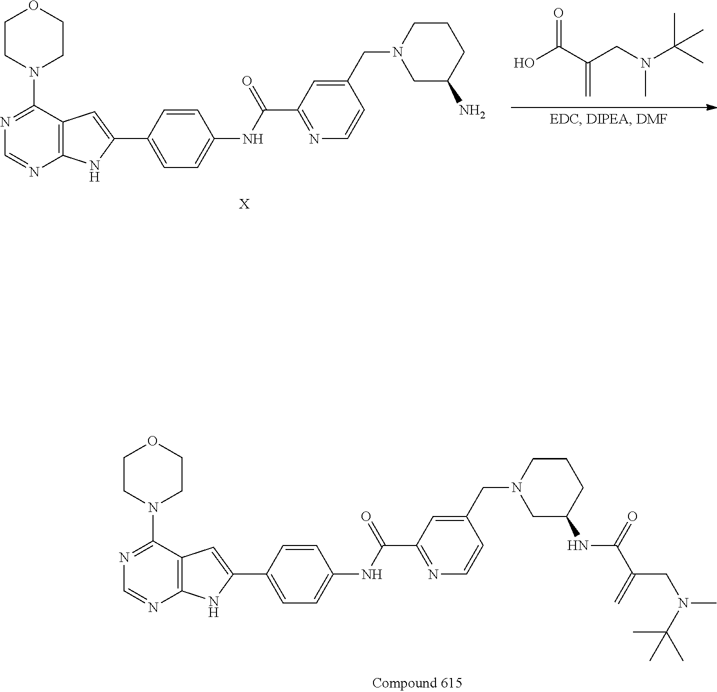

(R)-4-((3-aminopiperidin-1-yl)methyl)-N-(4-(4-morpholino-7H-pyrrolo[2,3-d]pyrimidin-6-yl)phenyl)picolinamide trihydrochloride X (75 mg, 0.121 mmol) was added to a solution of 1-(3-dimethylaminopropyl)-3-ethylcarbodiimide hydrochloride (35 mg, 0.181 mmol, 1.50 eq), 2-((tert-butyl(methyl)amino)methyl)acrylic acid (31 mg, 0.181 mmol) (as a solution in acetonitrile) and N,N-diisopropylethylamine (0.17 mL, 0.965 mmol) in N,N-dimethylformamide (5 mL). After 22 hours at 35° C. the reaction was subjected to preparative HPLC(5-85% acetonitrile/water, 0.1% trifluoroacetic acid). The fractions containing product were combined and subjected to lyophilization, providing Compound 615 (26 mg, 0.0320 mmol, 26%). ¹H NMR (400 MHz, MeOD) & 8.75 (d, J=5.0 Hz, 1H), 8.29 (s, 1H), 8.21 (s, 1H), 7.92-7.85 (m, 2H), 7.84-7.76 (m, 2H), 7.67 (dd, J=5.0, 1.7 Hz, 1H), 7.20 (s, 1H), 6.28 (s, 1H), 6.01 (s, 1H), 4.40 (s, 2H), 4.34 (m, 2H), 4.13 (m, 1H), 4.04-3.94 (m, 4H), 3.83 (m, 4H), 3.38 (m, 2H), 3.09-2.82 (m, 2H), 2.62 (s, 3H), 1.95 (m, 2H), 1.87-1.70 (m, 1H), 1.56 (m, 1H), 1.36 (s, 9H).

LCMS=[M+H]⁺: 666.50; Purity=94%.

### Example 33

### Compound 621

### Preparation of (R)-4-((3-(2-((3-fluoroazetidin-1-yl)methyl)acrylamido)piperidin-1-yl)methyl)-N-(4-(4-morpholino-7H-pyrrolo[2,3-d]pyrimidin-6-yl)phenyl)picolinamide (Compound 621)

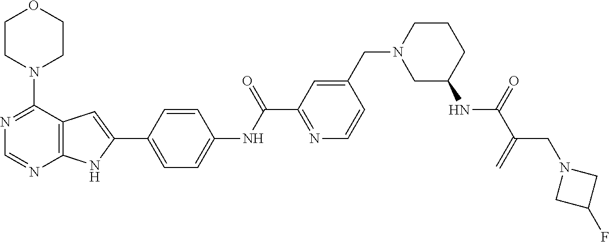 [SMILES: C=C(CN1CC(F)C1)C(=O)N[C@@H]1CCCN(Cc2ccnc(C(=O)Nc3ccc(-c4cc5c(N6CCOCC6)ncnc5[nH]4)cc3)c2)C1]

Step 1:2-((3-Fluoroazetidin-1-yl)methyl)acrylic acid

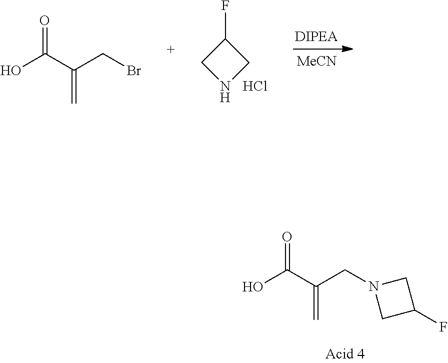

A solution of 2-(bromomethyl)prop-2-enoic acid (250 mg, 1.51 mmol), 3-fluoroazetidine hydrochloride (219 mg, 1.96 mmol), and N,N-diisopropylethylamine (0.79 mL, 4.54 mmol) in acetonitrile (4 mL) was stirred at 35° C. for 1 hour, providing a solution of 2-((3-fluoroazetidin-1-yl)methyl)acrylic acid (241 mg, 1.51 mmol, 99%) in acetonitrile. The reaction was assumed to be quantitative. Aliquots of this solution were used in the next step without workup.

Step 2: (R)-4-((3-(2-((3-fluoroazetidin-1-yl)methyl)acrylamido)piperidin-1-yl)methyl)-N-(4-(4-morpholino-7H-pyrrolo[2,3-d]pyrimidin-6-yl)phenyl)picolinamide (Compound 621)

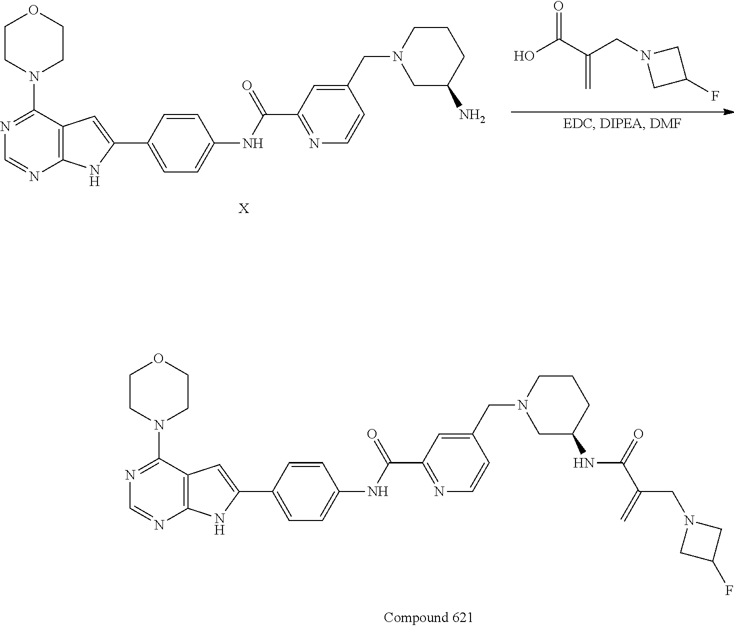

(R)-4-((3-aminopiperidin-1-yl)methyl)-N-(4-(4-morpholino-7H-pyrrolo[2,3-d]pyrimidin-6-yl)phenyl)picolinamide trihydrochloride X (75 mg, 0.121 mmol) was added to a solution of 1-(3-dimethylaminopropyl)-3-ethylcarbodiimide hydrochloride (35 mg, 0.181 mmol), 2-((3-fluoroazetidin-1-yl)methyl)acrylic acid (29 mg, 0.181 mmol) (as a solution in acetonitrile), and N,N-diisopropylethylamine (0.17 mL, 0.965 mmol) in N,N-dimethylformamide (5 mL). After 24 hours the reaction was subjected to preparative HPLC(5-85% acetonitrile/water, 0.1% trifluoroacetic acid). The fractions containing product were combined and subjected to lyophilization, providing Compound 621 (21 mg, 0.0259 mmol, 21%). ¹H NMR (400 MHz, MeOD) & 8.75 (d, J=4.9 Hz, 1H), 8.30 (d, J=1.7 Hz, 1H), 8.23 (d, J=7.7 Hz, 1H), 7.92-7.85 (m, 2H), 7.82-7.76 (m, 2H), 7.68 (dd, J=5.0, 1.7 Hz, 1H), 7.21 (s, 1H), 6.16 (s, 1H), 5.95 (s, 1H), 5.41-5.15 (m, 1H), 4.43 (m, 4H), 4.32-4.17 (m, 2H), 4.12 (m, 2H), 4.05-3.90 (m, 5H), 3.90-3.74 (m, 4H), 3.43 (m, 1H), 3.41-3.26 (m, 1H), 2.97 (m, 2H), 1.98 (m, 2H), 1.81 (m, 1H), 1.60 (m, 1H). LCMS=[M+H]⁺: 654.4; Purity=93%.

### Example 34

### Compound 628

### Preparation of (R)-4-((3-(2-((3-fluoro-3-(hydroxymethyl)azetidin-1-yl)methyl)acrylamido)piperidin-1-yl)methyl)-N-(4-(4-morpholino-7H-pyrrolo[2,3-d]pyrimidin-6-yl)phenyl)picolinamide (Compound 628)

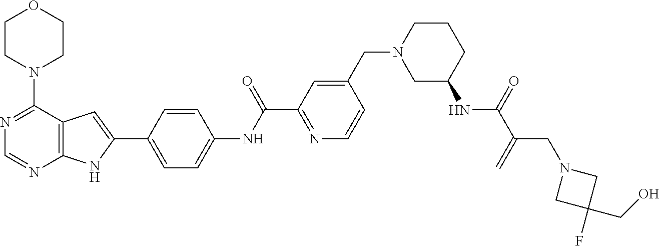 [SMILES: C=C(CN1CC(F)(CO)C1)C(=O)N[C@@H]1CCCN(Cc2ccnc(C(=O)Nc3ccc(-c4cc5c(N6CCOCC6)ncnc5[nH]4)cc3)c2)C1]

Step 1:2-((3-(hydroxymethyl)-3-methylazetidin-1-yl)methyl)acrylic acid

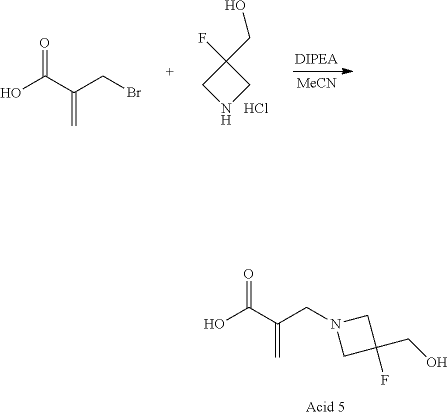

A solution of 2-(bromomethyl)prop-2-enoic acid (50 mg, 0.303 mmol), (3-fluoroazetidin-3-yl)methanol hydrochloride (51 mg, 0.363 mmol), and N,N-diisopropylethylamine (0.158 mL, 0.909 mmol) in acctonitrilc (4 mL) was stirred at 35° C. for 1 hour, providing a solution of 2-((3-fluoro-3-(hydroxymethyl)azetidin-1-yl)methyl)acrylic acid (57 mg, 0.301 mmol, 99%) in acetonitrile. The reaction was assumed to be quantitative. Aliquots of this solution were used in the next step without workup.

Step 2: (R)-4-((3-(2-((3-fluoro-3-(hydroxymethyl)azetidin-1-yl)methyl)acrylamido)piperidin-1-yl)methyl)-N-(4-(4-morpholino-7H-pyrrolo[2,3-d]pyrimidin-6-yl)phenyl)picolinamide (Compound 628)

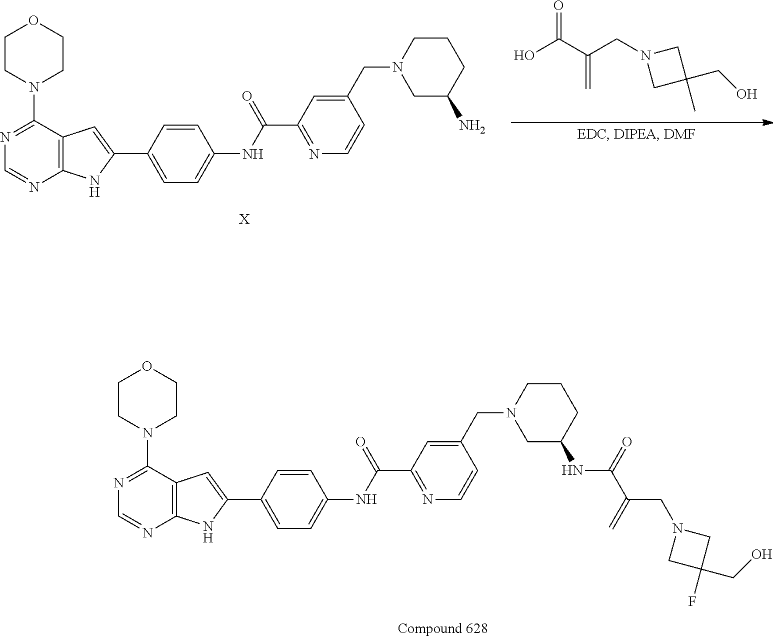

(R)-4-((3-aminopiperidin-1-yl)methyl)-N-(4-(4-morpholino-7H-pyrrolo[2,3-d]pyrimidin-6-yl)phenyl)picolinamide trihydrochloride X (75 mg, 0.121 mmol) was added to a solution of 1-(3-dimethylaminopropyl)-3-ethylcarbodiimide hydrochloride (14 mg, 0.0723 mmol), 2-((3-fluoro-3 -(hydroxymethyl)azetidin-1-yl)methyl)acrylic acid (14 mg, 0.0723 mmol) (as a solution in acetonitrile), and N,N-diisopropylethylamine (0.067 mL, 0.386 mmol) in N,N-dimethylformamide (2 mL). After 4 days at 32° C. the reaction was subjected to preparative HPLC(5-85% acetonitrile/water, 0.1% trifluoroacetic acid). The fractions containing product were combined and subjected to lyophilization, providing Compound 628 (29 mg, 0.0339 mmol, 70%). ¹H NMR (400 MHz, MeOD) & 8.75 (d, J=4.9 Hz, 1H), 8.29 (d, J=1.7 Hz, 1H), 8.21 (s, 1H), 7.92-7.83 (m, 2H), 7.83-7.74 (m, 2H), 7.67 (dd, J=5.0, 1.7 Hz, 1H), 7.19 (s, 1H), 6.17 (s, 1H), 5.94 (s, 1H), 4.40 (s, 2H), 4.28 (m, 4H), 4.20-4.04 (m, 2H), 4.04-3.89 (m, 5H), 3.83 (dd, J=5.8, 4.0 Hz, 4H), 3.66 (d, J=14.2 Hz, 2H), 3.40 (m, 1H), 3.32 (m, 1H), 3.07-2.60 (m, 2H), 1.98 (m, 2H), 1.79 (m, 1H), 1.58 (m, 1H). LCMS=[M+H]⁺: 684.4; Purity=95%.

### Example 35

### Compound 630

### Preparation of N-(4-(4-((1R,5S)-3-oxa-8-azabicyclo[3.2.1]octan-8-yl)-7H-pyrrolo[2,3-d]pyrimidin-6-yl)phenyl)-4-(((R)-3-(2-((methylsulfonyl)methyl)acrylamido)piperidin-1-yl)methyl)picolinamide (Compound 630)

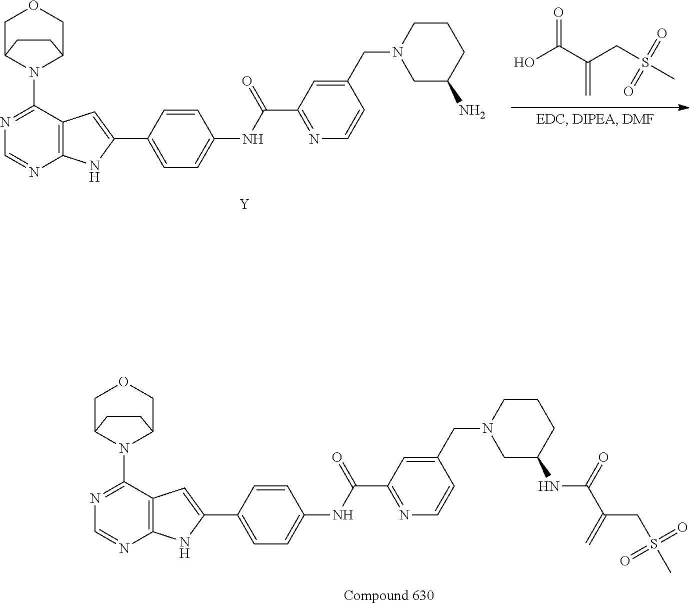

N,N-Diisopropylethylamine (0.052 mL, 0.297 mmol) was added to a solution of 1-(3-dimethylaminopropyl)-3-ethylcarbodiimide hydrochloride (13 mg, 0.0668 mmol), 2-(methylsulfonylmethyl)prop-2-enoic acid (11 mg, 0.0668 mmol), and N-(4-(4-((1R,5S)-3-oxa-8-azabicyclo[3.2.1]octan-8-yl)-7H-pyrrolo[2,3-d]pyrimidin-6-yl)phenyl)-4-(((R)-3-aminopiperidin-1-yl)methyl)picolinamide Y (20 mg, 0.0371 mmol) in N,N-dimethylformamide (5 mL) and heated at 32° C. After 18 hours the reaction mixture was subjected to preparative HPLC(5-85% acetonitrile/water, 0.1% trifluoroacetic acid). The fractions containing product were combined and subjected to lyophilization, providing N-(4-(4-((1R,5S)-3-oxa-8-azabicyclo[3.2.1]octan-8-yl)-7H-pyrrolo[2,3-d]pyrimidin-6-yl)phenyl)-4-(((R)-3-(2-((methylsulfonyl)methyl)acrylamido)piperidin-1-yl)methyl)picolinamide trifluoroacetate Compound 630 (4.9 mg, 0.00583 mmol, 15%). ¹H NMR (400 MHz, MeOD) ô 8.75 (d, J=4.9 Hz, 1H), 8.29 (d, J=1.6 Hz, 1H), 8.16 (s, 1H), 7.93-7.85 (m, 2H), 7.84-7.76 (m, 2H), 7.65 (dd, J=4.9, 1.7 Hz, 1H), 7.18 (s, 1H), 5.99 (s, 1H), 5.77 (s, 1H), 4.94 (m, 2H), 4.41 (m, 2H), 4.19-4.00 (m, 3H), 3.77 (q, J=11.3 Hz, 4H), 3.53-3.31 (m, 2H), 3.08-2.89 (m, 1H), 2.85 (s, 3H), 2.72 (m, 1H), 2.28-2.07 (m, 4H), 1.97 (m, 2H), 1.89-1.69 (m, 1H), 1.58 (m, 1H).

LCMS=[M+H]⁺: 685.30; Purity=94%.

### Example 36

### Compound 631

### Preparation of N-(4-(4-((1R,5S)-3-oxa-8-azabicyclo[3.2.1]octan-8-yl)-7H-pyrrolo[2,3-d]pyrimidin-6-yl)phenyl)-4-(((R)-3-(2-chloroacrylamido)piperidin-1-yl)methyl)picolinamide (Compound 631)

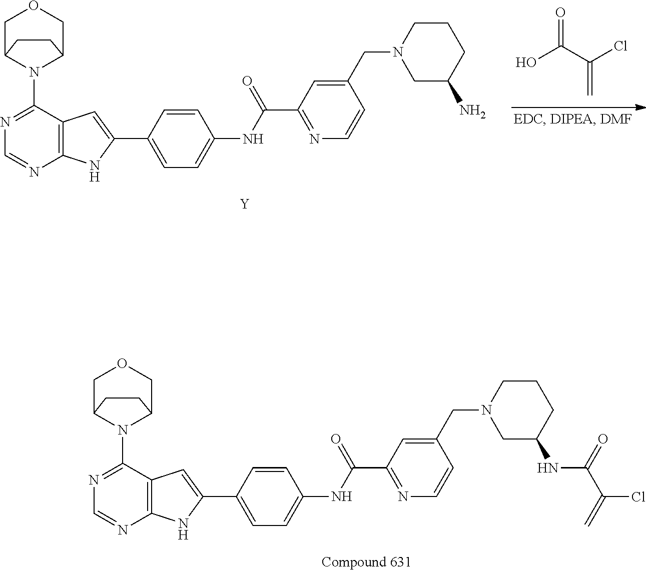

N,N-Diisopropylethylamine (0.052 mL, 0.297 mmol) was added to a solution of 1-(3-dimethylaminopropyl)-3-ethylcarbodiimide hydrochloride (13 mg, 0.0668 mmol), 2-chloroprop-2-enoic acid (6 mg, 0.0557 mmol), and N-(4-(4-((1R,5S)-3-oxa-8-azabicyclo[3.2.1]octan-8-yl)-7H-pyrrolo[2,3-d]pyrimidin-6-yl)phenyl)-4-(((R)-3-aminopiperidin-1-yl)methyl)picolinamide Y (20 mg, 0.0371 mmol) in N,N-dimethylformamide (5 mL) and heated at 32° C. After 18 hours the reaction mixture was subjected to preparative HPLC(5-85% acetonitrile/water, 0.1% trifluoroacetic acid). The fractions containing product were combined and subjected to lyophilization, providing N-(4-(4-((1R,5S)-3-oxa-8-azabicyclo[3.2.1]octan-8-yl)-7H-pyrrolo[2,3-d]pyrimidin-6-yl)phenyl)-4-(((R)-3-(2-chloroacrylamido)piperidin-1-yl)methyl)picolinamide trifluoroacetate Compound 631 (3.7 mg, 0.00474 mmol, 12%). ¹H NMR (400 MHz, MeOD) & 8.76 (d, J=4.9 Hz, 1H), 8.30 (t, J=1.2 Hz, 1H), 8.16 (s, 1H), 7.93-7.85 (m, 2H), 7.84-7.76 (m, 2H), 7.66 (dd, J=5.0, 1.7 Hz, 1H), 7.20 (s, 1H), 6.30 (d, J=1.9 Hz, 1H), 5.81 (d, J=1.9 Hz, 1H), 4.94 (m, 2H), 4.41 (s, 2H), 4.11 (m, 1H), 3.78 (q, J=11.3 Hz, 4H), 3.40 (m, 2H), 3.07-2.80 (m, 2H), 2.26-2.05 (m, 4H), 1.95 (m, 2H), 1.87-1.69 (m, 1H), 1.63 (m, 1H).

LCMS=[M+H]⁺: 627.30; Purity=95%.

### Example 37

### Compound 632

### Preparation of N-(4-(4-((1R,5S)-3-oxa-8-azabicyclo[3.2.1]octan-8-yl)-7H-pyrrolo[2,3-d]pyrimidin-6-yl)phenyl)-4-(((R)-3-((E)-4-methoxybut-2-enamido)piperidin-1-yl)methyl)picolinamide (Compound 632)

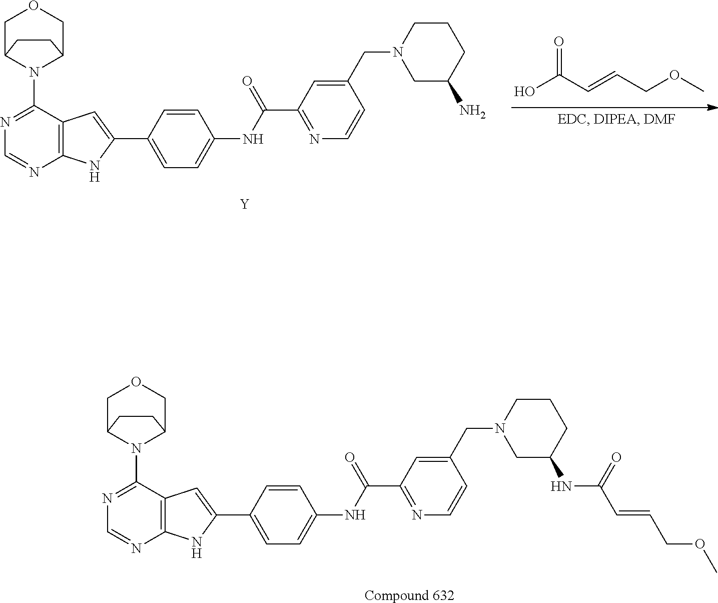

N,N-Diisopropylethylamine (0.052 mL, 0.297 mmol) was added to a solution of 1-(3-dimethylaminopropyl)-3-ethylcarbodiimide hydrochloride (13 mg, 0.0668 mmol), (E)-4-methoxybut-2-enoic acid (6 mg, 0.0557 mmol), and N-(4-(4-((1R,5S)-3-oxa-8-azabicyclo[3.2.1]octan-8-yl)-7H-pyrrolo[2,3-d]pyrimidin-6-yl)phenyl)-4-(((R)-3-aminopiperidin-1-yl)methyl)picolinamide Y (20 mg, 0.0371 mmol) in N,N-dimethylformamide (5 mL) and heated at 32° C. After 18 hours the reaction mixture was subjected to preparative HPLC(5-85% acetonitrile/water, 0.1% trifluoroacetic acid). The fractions containing product were combined and subjected to lyophilization, providing of N-(4-(4-((1R,5S)-3-oxa-8-azabicyclo[3.2.1]octan-8-yl)-7H-pyrrolo[2,3-d]pyrimidin-6-yl)phenyl)-4-(((R)-3-((E)-4-methoxybut-2-enamido)piperidin-1-yl)methyl)picolinamide trifluoroacetate Compound 632 (9.5 mg, 0.012 mmol, 32%). ¹H NMR (400 MHz, MeOD) & 8.76 (d, J=4.9 Hz, 1H), 8.32-8.27 (m, 1H), 8.16 (s, 1H), 7.92-7.84 (m, 2H), 7.83-7.75 (m, 2H), 7.66 (dd, J=5.1, 1.8 Hz, 1H), 7.19 (s, 1H), 6.69 (dt, J=15.5, 4.3 Hz, 1H), 6.04 (dt, J=15.5, 2.0 Hz, 1H), 4.95 (m, 2H), 4.42 (m, 2H), 4.12-3.99 (m, 1H), 3.97 (dd, J=4.3, 2.0 Hz, 2H), 3.77 (q, J=11.3 Hz, 4H), 3.41 (m, 2H), 3.27 (s, 3H), 3.03 (m, 1H), 2.91-2.66 (m, 1H), 2.27-2.07 (m, 4H), 1.97 (m, 2H), 1.87-1.70 (m, 1H), 1.54 (m, 1H).

LCMS=[M+H]⁺: 637.40; Purity=95%.

### Example 38

### Compound 633

### Preparation of N-(4-(4-((1R,5S)-3-oxa-8-azabicyclo[3.2.1]octan-8-yl)-7H-pyrrolo[2,3-d]pyrimidin-6-yl)phenyl)-4-(((R)-3-((E)-4-(dimethylamino)but-2-enamido)piperidin-1-yl)methyl)picolinamide (Compound 633)

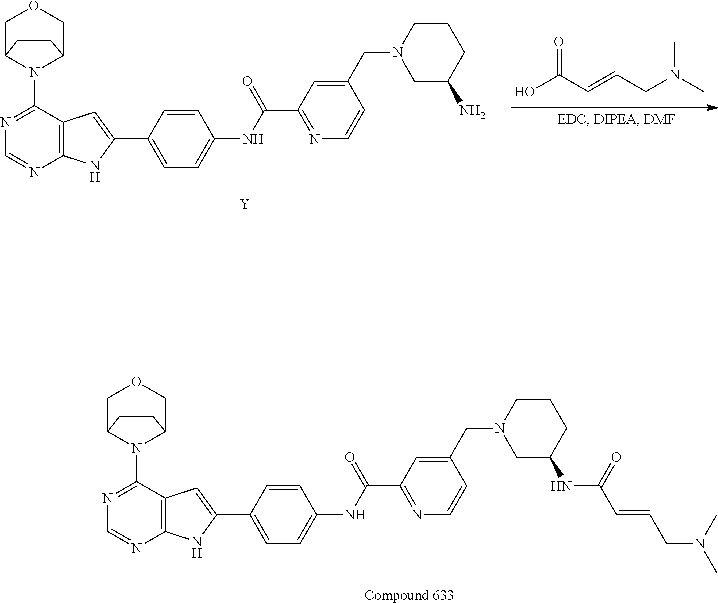

N,N-Diisopropylethylamine (0.052 mL, 0.297 mmol) was added to a solution of 1-(3-dimethylaminopropyl)-3-ethylcarbodiimide hydrochloride (13 mg, 0.0668 mmol), (E)-4-(dimethylamino)but-2-enoic acid (7.2 mg, 0.0557 mmol), and N-(4-(4-((1R,5S)-3-oxa-8-azabicyclo[3.2.1]octan-8-yl)-7H-pyrrolo[ 2,3-d]pyrimidin-6-yl)phenyl)-4-(((R)-3-aminopiperidin-1-yl)methyl)picolinamide Y (20 mg, 0.0371 mmol) in N,N-dimethylformamide (5 mL) and heated at 32° C. After 18 hours the reaction mixture was subjected to preparative HPLC(5-85% acetonitrile/water, 0.1% trifluoroacetic acid). The fractions containing product were combined and subjected to lyophilization, providing of N-(4-(4-((1R,5S)-3-oxa-8-azabicyclo[3.2.1]octan-8-yl)-7H-pyrrolo[2,3-d]pyrimidin-6-yl)phenyl)-4-(((R)-3-((E)-4-(dimethylamino)but-2-enamido)piperidin-1-yl)methyl)picolinamide trifluoroacetate Compound 633 (9.5 mg, 0.012 mmol, 32%). ¹H NMR (400 MHz, MeOD) & 8.74 (d, J=4.9 Hz, 1H), 8.28 (s, 1H), 8.16 (s, 1H), 7.92-7.84 (m, 2H), 7.84-7.77 (m, 2H), 7.68-7.62 (m, 1H), 7.18 (s, 1H), 6.62 (dt, J=14.9, 7.3 Hz, 1H), 6.26 (d, J=15.3 Hz, 1H), 4.94 (m, 2H), 4.34 (s, 2H), 4.06 (m, 1H), 3.88-3.70 (m, 6H), 3.44-3.33 (m, 2H), 3.05-2.86 (m, 2H), 2.78 (s, 6H), 2.26-2.07 (m, 4H), 1.94 (m, 2H), 1.77 (m, 1H), 1.53 (m, 1H).

LCMS=[M+H]⁺: 650.40; Purity=94%.

### Example 39

### Compound 637

### Preparation of (R)-4-((3-(1-methyl-1,2,5,6-tetrahydropyridine-3-carboxamido)piperidin-1-yl)methyl)-N-(4-(4-morpholino-7H-pyrrolo[2,3-d]pyrimidin-6-yl)phenyl)picolinamide (Compound 637)

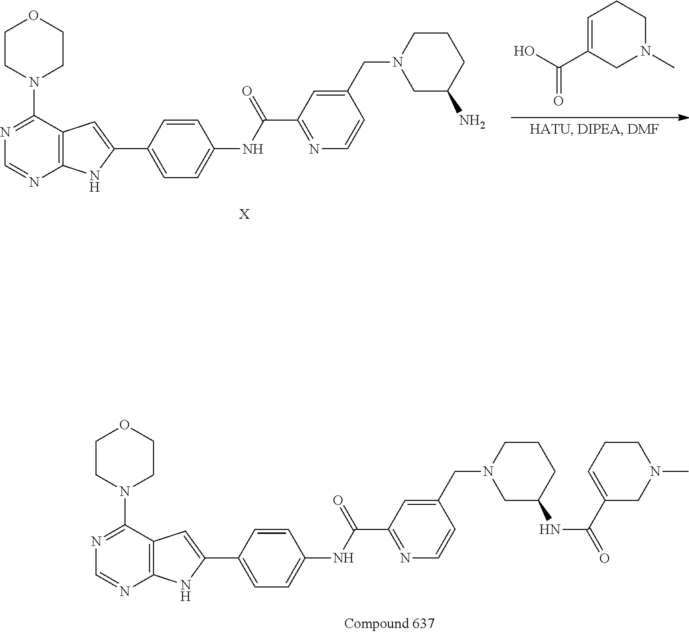

(R)-4-((3-aminopiperidin-1-yl)methyl)-N-(4-(4-morpholino-7H-pyrrolo[2,3-d]pyrimidin-6-yl)phenyl)picolinamide trihydrochloride trihydrochloride X (50 mg, 0.0804 mmol) was added to a solution of O-(7-azabenzotriazol-1-yl)-N,N,N′,N′-tetramethyluronium hexafluorophosphate (40 mg, 0.105 mmol), arecaidine hydrochloride (19 mg, 0.105 mmol), and N,N-diisopropylethylamine (0.11 mL, 0.643 mmol) in N,N-dimethylformamide (5 mL). After 2 hours the reaction was subjected to preparative HPLC(5-85% acetonitrile/water, 0.1% trifluoroacetic acid). The fractions containing product were combined and subjected to lyophilization, providing (R)-4-((3-(1-methyl-1,2,5,6-tetrahydropyridine-3-carboxamido)piperidin-1-yl)methyl)-N-(4-(4-morpholino-7H-pyrrolo[2,3-d]pyrimidin-6-yl)phenyl)picolinamide trifluoroacete Compound 637 (38 mg, 0.0478 mmol, 59%). ¹H NMR (400 MHz, MeOD) & 8.75 (d, J=4.9 Hz, 1H), 8.29 (d, J=1.3 Hz, 1H), 8.21 (s, 1H), 7.93-7.85 (m, 2H), 7.83-7.74 (m, 2H), 7.66 (dd, J=5.0, 1.8 Hz, 1H), 7.21 (s, 1H), 6.72 (s, 1H), 4.42 (s, 2H), 4.08 (m, 1H), 3.99 (dd, J=5.9, 4.0 Hz, 4H), 3.83 (dd, J=5.8, 4.0 Hz, 4H), 3.64 (m, 1H), 3.44 (m, 3H), 3.04 (m, 2H), 2.88 (s, 3H), 2.55 (s, 2H), 2.05-1.96 (m, 2H), 1.92 (m, 2H), 1.79 (m, 1H), 1.60 (m, 1H).

LCMS=[M+H]⁺: 636.40; Purity=95%.

### Example 40

### Compound 638

### Preparation of N-(4-(4-((1R,5S)-3-oxa-8-azabicyclo[3.2.1]octan-8-yl)-7H-pyrrolo[2,3-d]pyrimidin-6-yl)phenyl)-4-(((R)-3-((E)-4-(dimethylamino)but-2-enamido)piperidin-1-yl)methyl)picolinamide (Compound 638)

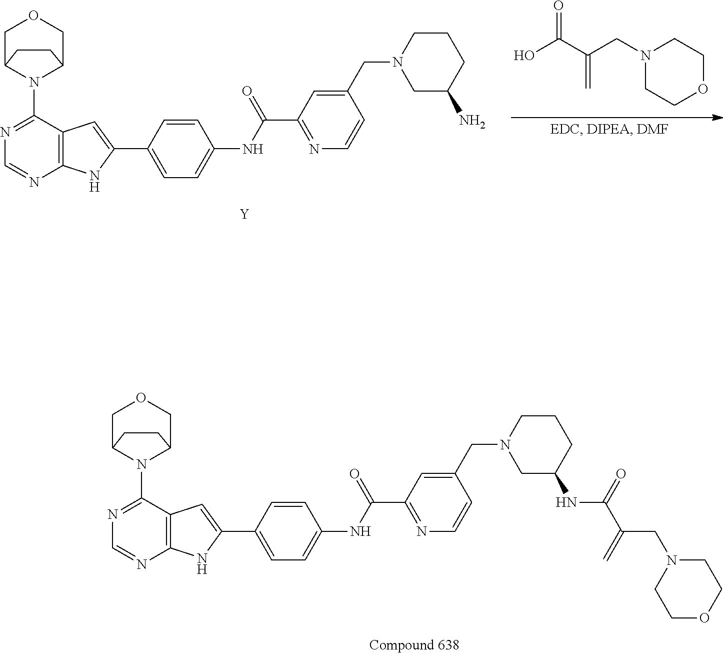

N,N-Diisopropylethylamine (0.026 mL, 0.149 mmol) was added to a solution of 1-(3-dimethylaminopropyl)-3-ethylcarbodiimide hydrochloride (6.4 mg, 0.0334 mmol), 2-(morpholinomethyl)prop-2-enoic acid (5.7 mg, 0.0334 mmol), and N-(4-(4-((1R,5S)-3-oxa-8-azabicyclo[3.2.1]octan-8-yl)-7H-pyrrolo[2,3-d]pyrimidin-6-yl)phenyl)-4-(((R)-3-aminopiperidin-1-yl)methyl)picolinamide Y (10 mg, 0.0186 mmol) in N,N-dimethylformamide (5 mL) and heated at 32° C. After 18 hours the reaction mixture was subjected to preparative HPLC(5-85% acetonitrile/water, 0.1% trifluoroacetic acid). The fractions containing product were combined and subjected to lyophilization, providing of N-(4-(4-((1R,5S)-3-oxa-8-azabicyclo[3.2.1]octan-8-yl)-7H-pyrrolo[2,3-d]pyrimidin-6-yl)phenyl)-4-(((R)-3-(2-(morpholinomethyl)acrylamido)piperidin-1-yl)methyl)picolinamide trifluoroacetate Compound 638 (4.4 mg, 0.00508 mmol, 27%). ¹H NMR (400 MHz, MeOD) ô 8.74 (d, J=4.9 Hz, 1H), 8.29 (s, 1H), 8.16 (s, 1H), 7.93-7.85 (m, 2H), 7.85-7.76 (m, 2H), 7.66 (dd, J=5.0, 1.7 Hz, 1H), 7.19 (s, 1H), 6.23 (s, 1H), 5.97 (s, 1H), 4.94 (m, 2H), 4.37 (s, 2H), 4.08 (m, 1H), 3.95-3.83 (m, 2H), 3.77 (m, 8H), 3.42 (m, 1H), 3.32 (m, 1H), 3.17 (m, 4H), 2.91 (m, 2H), 2.27-2.06 (m, 4H), 1.98 (m, 2H), 1.80 (m, 1H), 1.59 (m, 1H).

LCMS=[M+H]⁺: 692.40; Purity=93%.

### Example 41

### Compound 639

### Preparation of N-(4-(4-((1R,5S)-3-oxa-8-azabicyclo[3.2.1]octan-8-yl)-7H-pyrrolo[2,3-d]pyrimidin-6-yl)phenyl)-4-(((R)-3-(2-((dimethylamino)methyl)acrylamido)piperidin-1-yl)methyl)picolinamide (Compound 639)

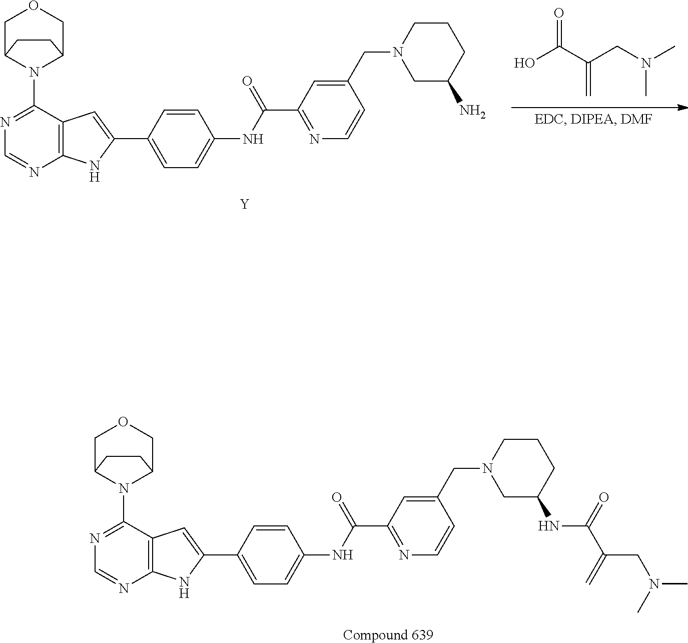

N,N-Diisopropylethylamine (0.026 mL, 0.149 mmol) was added to a solution of 1-(3-dimethylaminopropyl)-3-cthylcarbodiimide hydrochloride (6.4 mg, 0.0334 mmol), 2-[(dimethylamino)methyl]prop-2-enoic acid (4.3 mg, 0.0334 mmol), and N-(4-(4-((1R,5S)-3-oxa-8-azabicyclo[3.2.1]octan-8-yl)-7H-pyrrolo[2,3-d]pyrimidin-6-yl)phenyl)-4-(((R)-3-aminopiperidin-1-yl)methyl)picolinamide Y (10 mg, 0.0186 mmol) in N,N-dimethylformamide (5 mL) and heated at 32° C. After 18 hours the reaction mixture was subjected to preparative HPLC(5-85% acetonitrile/water, 0.1% trifluoroacetic acid). The fractions containing product were combined and subjected to lyophilization, providing of N-(4-(4-((1R,5S)-3-oxa-8-azabicyclo[3.2.1]octan-8-yl)-7H-pyrrolo[2,3-d]pyrimidin-6-yl)phenyl)-4-(((R)-3-(2-((dimethylamino)methyl)acrylamido)piperidin-1-yl)methyl)picolinamide trifluoroacetate Compound 639 (5.9 mg, 0.0072 mmol, 38%). ¹H NMR (400 MHz, MeOD) δ 8.74 (d, J=4.9 Hz, 1H), 8.29 (s, 1H), 8.16 (s, 1H), 7.88 (d, J=8.7 Hz, 2H), 7.81 (d, J=8.7 Hz, 2H), 7.68-7.63 (m, 1H), 7.19 (s, 1H), 6.22 (s, 1H), 5.96 (s, 1H), 4.94 (m, 2H), 4.35 (s, 2H), 4.11 (m, 1H), 3.90-3.81 (m, 2H), 3.81-3.67 (m, 4H), 3.38 (m, 1H), 3.27 (m, 1H), 2.98 (m, 2H), 2.76 (s, 6H), 2.28-2.04 (m, 4H), 2.04-1.87 (m, 2H), 1.78 (m, 1H), 1.58 (m, 1H).

LCMS=[M+H]⁺: 650.40; Purity=93%.

### Example 42

### Compound 634

### Preparation of 3-(4-(4-morpholino-7H-pyrrolo[2,3-d]pyrimidin-6-yl)phenyl)-7-(2-(morpholinomethyl) acryloyl)-1,3,7-triazaspiro[4.4]nonane-2,4-dione (Compound 634)

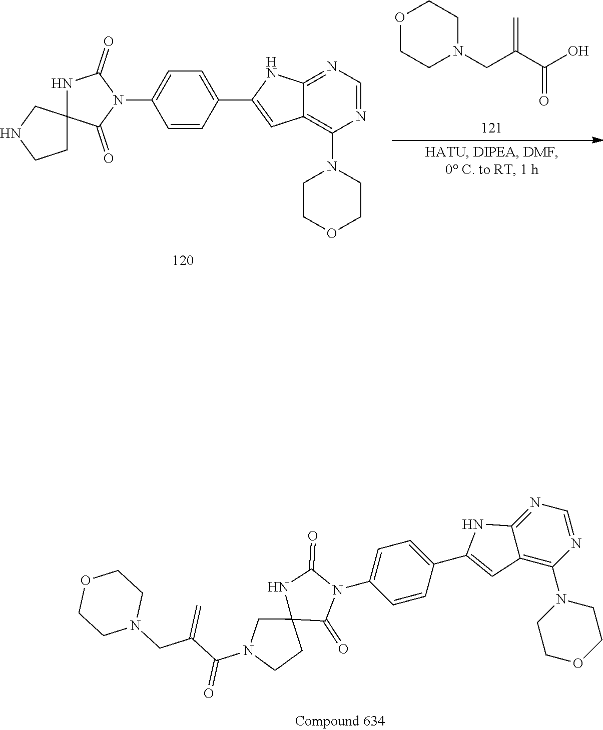 [SMILES: O=C1NC2(CCNC2)C(=O)N1c1ccc(-c2cc3c(N4CCOCC4)ncnc3[nH]2)cc1 ; C=C(CN1CCOCC1)C(=O)O ; C=C(CN1CCOCC1)C(=O)N1CCC2(C1)NC(=O)N(c1ccc(-c3cc4c(N5CCOCC5)ncnc4[nH]3)cc1)C2=O]

### Synthesis of 3-(4-(4-morpholino-7H-pyrrolo[2,3-d]pyrimidin-6-yl)phenyl)-7-(2-(morpholinomethyl) acryloyl)-1,3,7-triazaspiro[4.4]nonane-2,4-dione (Compound 634)

To a stirred solution of 3-[p-(4-morpholino-1H-1,5,7-triazainden-2-yl)phenyl]-1,3,7-triaza-2,4-spiro[4.4]nonanedione 120 (300 mg, 0.692 mmol) and 2-(morpholinomethyl)acrylic acid 121 (118 mg, 0.692 mmol) in DMF (5 mL) were added DIPEA (0.38 mL, 2.08 mmol) and HATU (526 mg, 1.38 mmol) sequentially at 0° C. under N₂ atmosphere. The reaction was stirred at RT for 1 h. After completion of reaction (TLC monitoring), the reaction mixture was poured into ice cold water (50 mL) and extracted with 25% IPA in CHCl₃ (3×50 mL). The combined organic layer was washed with ice brine (2×50 mL), dried over Na₂SO₄, filtered and concentrated under reduced pressure. The crude was purified by RP HPLC [5-95% 5 mM ammonium bicarbonate in water and/ACN; Column: X Select Phenyl Hexyl (19*250) 5μ] to get the Compound 634 (105 mg, 26%).

¹H NMR (400 MHz, DMSO-d₆): δ 12.29 (s, 1H), 9.03 (s, 1H), 8.19 (s, 1H), 8.01-7.98 (m, 2H), 7.49-7.44 (m, 2H), 7.26 (s, 1H), 5.45 (d, J=11.2 Hz, 1H), 5.37 (d, J=18 Hz, 1H), 3.91-3.88 (m, 4H), 3.76-3.74 (m, 6H), 3.61-3.55 (m, 6H), 3.19-3.15 (m, 1H), 3.09-3.03 (m, 1H), 2.43-2.35 (m, 5H), 2.21-2.16 (m, 1H). LCMS=[M+H]⁺: 587.41; Purity=96.32%.

### Example 43

### Compound 802

### Preparation of (R)-2-(1-((2-(((4-(4-morpholino-7H-pyrrolo[2,3-d]pyrimidin-6-yl)phenyl)amino)methyl)pyridin-4-yl)methyl)piperidin-3-yl)-2,3-dihydroisothiazole 1,1-dioxide (Compound 802)

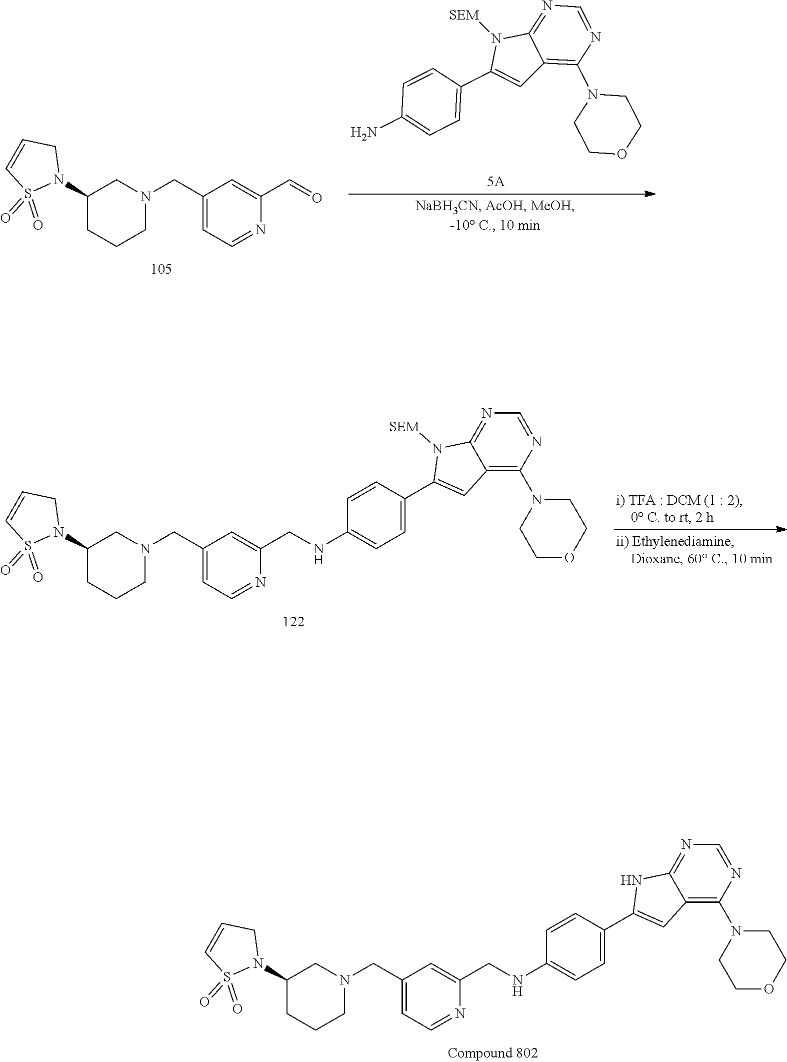 [SMILES: O=Cc1cc(CN2CCC[C@@H](N3CC=CS3(=O)=O)C2)ccn1 ; C[Si](C)(C)CCOCn1c(-c2ccc(N)cc2)cc2c(N3CCOCC3)ncnc21 ; C[Si](C)(C)CCOCn1c(-c2ccc(NCc3cc(CN4CCC[C@@H](N5CC=CS5(=O)=O)C4)ccn3)cc2)cc2c(N3CCOCC3)ncnc21 ; O=S1(=O)C=CCN1[C@@H]1CCCN(Cc2ccnc(CNc3ccc(-c4cc5c(N6CCOCC6)ncnc5[nH]4)cc3)c2)C1]

### Synthesis of (R)-2-(1-((2-(((4-(4-morpholino-7-((2-(trimethylsilyl)ethoxy)methyl)-7H-pyrrolo[2,3-d]pyrimidin-6-yl)phenyl)amino)methyl)pyridin-4-yl)methyl)piperidin-3-yl)-2,3-dihydroisothiazole 1,1-dioxide (122)

To a stirred solution of 4-{[(R)-3-(1,1-dioxo-1,2-dihydro-126-2-isothiazolyl)-1-piperidyl]methyl}-2-pyridinecarbaldehyde 105 (453 mg, 1.41 mmol) and p-(4-morpholino-1-{[2-(trimethylsilyl)ethoxy]methyl}-1H-1,5,7-triazainden-2-yl) aniline 5A (0.4 g, 0.940 mmol) in MeOH (20 mL) was added AcOH (cat) at −10° C. and stirred for 10 min. Then, NaCNBH₃ (112 mg, 1.88 mmol) was added to the reaction mixture at −10° C. and stirred for 10 min. After completion of reaction (LCMS monitoring), the reaction mixture was quenched with sat. NaHCO₃ and extracted with DCM (3×50 mL). The combined organic layer was dried over Na₂SO₄, filtered and concentrated under reduce pressure. The crude was purified by flash chromatography (silica gel 100-200 M, 12 g SNAP) using 2-3% MeOH in DCM to get the desired product as brown solid 122 (600 mg, 68%). LCMS=[M+H]⁺: 731.63; Purity=83%.

### Synthesis of (R)-2-(1-((2-(((4-(4-morpholino-7H-pyrrolo[2,3-d]pyrimidin-6-yl)phenyl)amino)methyl)pyridin-4-yl)methyl)piperidin-3-yl)-2,3-dihydroisothiazole 1,1-dioxide (Compound 802)

To a stirred solution of [(4-{[3-(1,1-dioxo-1,2-dihydro-126-2-isothiazolyl)-1-piperidyl]methyl}-2-pyridyl)methyl][p-(4-morpholino-1-{[2-(trimethylsilyl)ethoxy]methyl}-1H-1,5,7-triazainden-2-yl)phenyl]amine 122 (600 mg, 0.788 mmol) in DCM (5 mL) was added TFA (2.5 mL) dropwise at 0° C. and stirred at RT for 2 h. After completion of reaction (TLC and LCMS monitoring), the reaction mixture was concentrated under reduced pressure. The crude was dissolved in 1,4-dioxane (10 mL), followed by 1,2-ethanediamine (0.16 mL, 2.37 mmol) was added to the reaction mixture and stirred at 70° C. for 10 min. After completion of reaction (LCMS monitoring), the reaction mixture was diluted with water (50 mL) and extracted with 25% IPA in CHCl₃ (3×50 mL). The combined organic layer was dried over Na₂SO₄, filtered and concentrated under reduced pressure. The crude was purified by RP-HPLC [5-95% 5 mM ammonium bicarbonate in water/ACN; Column: X Select Phenyl Hexyl (19*250) 5μ] to get the Compound 802 (500 mg, 63%).

¹H NMR (400 MHz, DMSO-d₆): δ 11.91 (s, 1H), 8.46 (d, J=5.2 Hz, 1H), 8.11 (s, 1H), 7.60 (d, J=8.8 Hz, 2H), 7.32 (s, 1H), 7.18 (d, J=4.8 Hz, 1H), 7.09-7.04 (m, 2H), 6.83 (d, J=1.6 Hz, 1H), 6.62 (d, J=8.8 Hz, 2H), 6.57 (t, J=6 Hz, 1H), 4.39 (d, J=6 Hz, 2H), 4.04-3.96 (m, 2H), 3.84-3.81 (m, 4H), 3.73-3.71 (m, 4H), 3.38-3.35 (m, 3H), 2.88-2.85 (m, 1H), 2.57-2.54 (m, 1H), 2.15 (t, J=10.4 Hz, 1H), 1.89-1.80 (m, 2H), 1.62-1.59 (m, 1H), 1.52-1.38 (m, 2H).

LCMS=[M+H]⁺: 601.36; Purity=99.67%.

### Example 44

### Compound 635 and Compound 636

### Preparation of 3-(4-(4-morpholino-7H-pyrrolo[2,3-d]pyrimidin-6-yl)phenyl)-7-(2-(morpholinomethyl) acryloyl)-1,3,7-triazaspiro[4.5]decane-2,4-dione (Compound 635 and Compound 636)

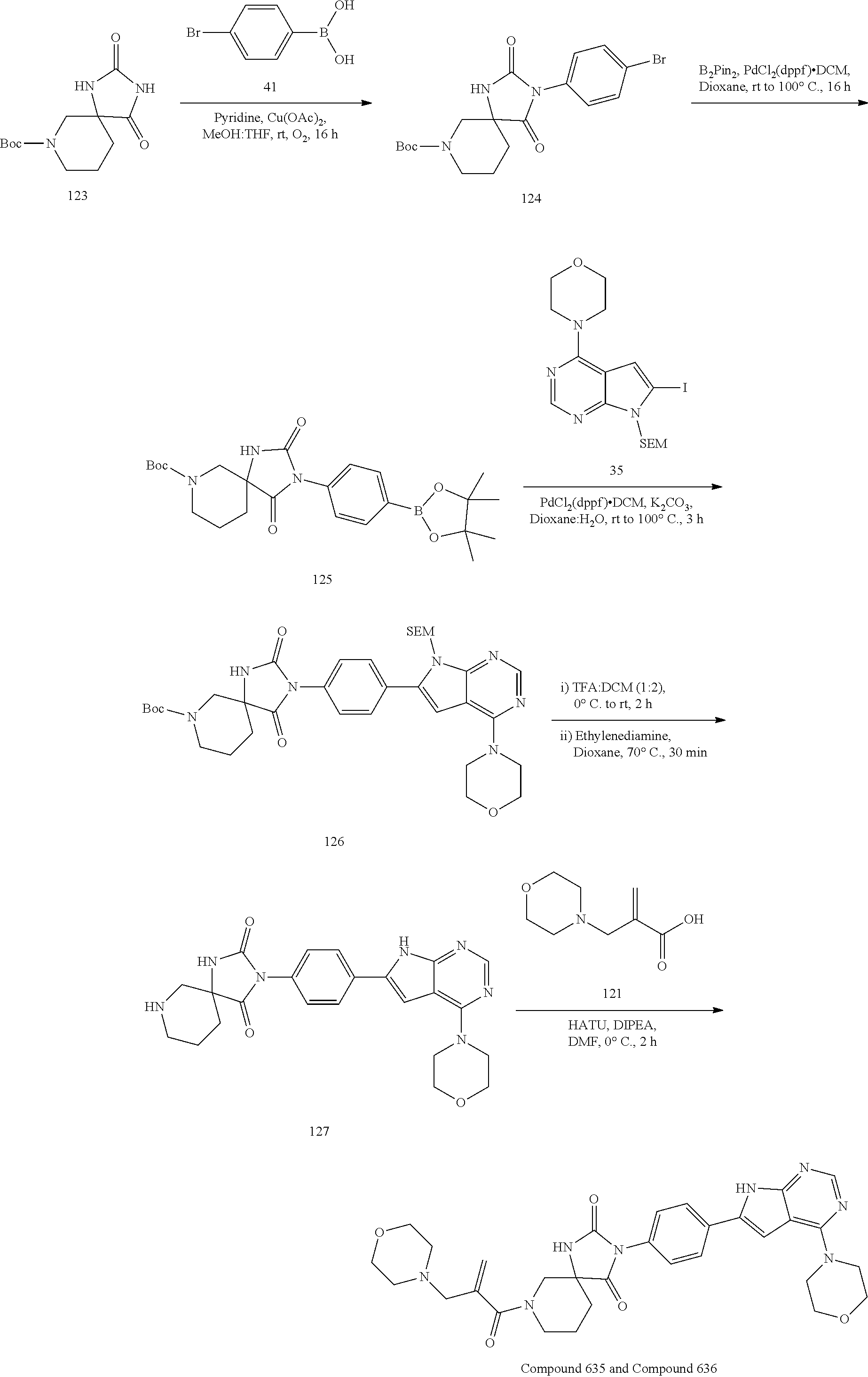

### Synthesis of tert-butyl 3-(4-bromophenyl)-2,4-dioxo-1,3,7-triazaspiro[4.5]decane-7-carboxylate (124)

To a stirred solution of tert-butyl 2,4-dioxo-1,3,7-triaza-7-spiro[4.5]decanecarboxylate 123 (1 g, 3.71 mmol) and 4-bromo phenylboronic acid 41 (1.12 g, 5.57 mmol) in MeOH (10 mL) and THF (10 mL) was added pyridine (0.9 mL, 11.1 mmol) at RT. The reaction mixture was purged with O₂ gas for 15 min. Then, Cu(OAc) 2 (1.01 g, 5.57 mmol) was added to the reaction mixture and stirred at RT for 16 h under 02 atmosphere. After completion of reaction (LCMS monitoring), the reaction mixture was filtered through Celite pad and washed with EtOAc (100 mL). The filtrate was washed with water (3×50 mL) and brine (2×50 mL). The organic layer was dried over Na₂SO₄, filtered and evaporated under reduced pressure. The crude was purified by flash chromatography (silica gel 100-200 M, 40 g SNAP) using 45-50% EtOAc in heptane to get the title compound as a white solid 124 (1 g, 63%).

LCMS=[M+H]⁺: 424.25; Purity=96.45%.

### Synthesis of tert-butyl 2,4-dioxo-3-(4-(4,4,5,5-tetramethyl-1,3,2-dioxaborolan-2-yl)phenyl)-1,3,7-triazaspiro[4.5]decane-7-carboxylate (125)

To a stirred solution of 3-(4-bromophenyl)-2,4-dioxo-1,3,7-triazaspiro[4.5]decane-7-carboxylate 124 (1 g, 2.36 mmol) in 1,4-dioxane (10 mL) were added B₂Pin₂ (718 mg, 2.83 mmol) and KOAc (694 mg, 7.07 mmol) at RT under N₂ atmosphere. The reaction mixture was degassed with N₂ gas for 15 min. After that, PdCl₂(dppf).DCM (192 mg, 0.236 mmol) was added to the reaction mixture and stirred at 100° C. for 16 h. After completion of reaction (monitoring by TLC), the reaction mixture was filtered through Celite pad and washed with EtOAc (100 mL). The filtrate was concentrated under reduced pressure to get the title compound as brown sticky solid 125 (1.3 g; crude) which was used in next step without further purification.

LCMS=[M−H]: 470.32; Purity=96.45%.

### Synthesis of tert-butyl 3-(4-(4-morpholino-7-((2-(trimethylsilyl)ethoxy)methyl)-7H-pyrrolo[2,3-d]pyrimidin-6-yl)phenyl)-2,4-dioxo-1,3,7-triazaspiro[4.5]decane-7-carboxylate (126)

To a stirred solution of tert-butyl 2,4-dioxo-3-(4-(4,4,5,5-tetramethyl-1,3,2-dioxaborolan-2-yl)phenyl)-1,3,7-triazaspiro[4.5]decane-7-carboxylate 125 (1.23 g, 2.61 mmol) and {2-[(2-iodo-4-morpholino-1H-1,5,7-triazainden-1-yl)methoxy]ethyl}tris(methyl) silane 35 (1 g, 2.17 mmol) in 1,4-dioxane (15 mL) and water (5 mL) was added K₂CO₃ (0.5 g, 3.62 mmol) under N₂ atmosphere at RT. The reaction mixture was degassed with N₂ gas for 15 min. Then, PdCl₂(dppf).DCM (177 mg, 0.217 mmol) was added to the reaction mixture and stirred at 100° C. for 3 h. After completion of reaction (TLC and LCMS monitoring), the reaction mixture was diluted with EtOAc (100 mL) and washed with water (2×50 mL). The organic layer was dried over Na₂SO₄, filtered and evaporated under reduced pressure. The crude was purified by flash chromatography (silica gel 100-200 M, 40 g SNAP) using 30-40% EtOAc in heptane to get the desired compound as a brown solid 126 (1 g, 67%).

LCMS=[M+H]⁺: 678.53; Purity=91%.

### Synthesis of 3-(4-(4-morpholino-7H-pyrrolo[2,3-d]pyrimidin-6-yl)phenyl)-1,3,7-triazaspiro[4.5]decane-2,4-dione (127)

To a stirred solution of tert-butyl 3-[p-(4-morpholino-1-{[2-(trimethylsilyl)ethoxy]methyl}-1H-1,5,7-triazainden-2-yl)phenyl]-2,4-dioxo-1,3,7-triaza-7-spiro[4.5]decanecarboxylate 126 (1 g, 1.48 mmol) in DCM (10 mL) was added TFA (5 mL) dropwise at 0° C. The reaction mixture was stirred at RT for 2 h. After completion of reaction (TLC and LCMS monitoring), the reaction mixture was concentrated under reduced pressure, triturated with diethyl ether and dried under reduced pressure. The crude was dissolved in 1,4-dioxane (10 mL), followed by addition of 1,2-ethanediamine (0.3 mL, 4.43 mmol) at RT and stirred at 70° C. for 30 min. After completion of reaction (monitoring by LCMS), the reaction was poured into ice cold water (20 mL) and extracted with 25% IPA in CHCl₃ (3×50 mL). The combined organic layer was dried over Na₂SO₄, filtered and concentrated under reduced pressure to get the title compound as a yellow solid 127 (600 mg; crude) which was used in next step without further purification.

LCMS=[M+H]⁺: 448.26; Purity=78%.

### Synthesis of 3-(4-(4-morpholino-7H-pyrrolo[2,3-d]pyrimidin-6-yl)phenyl)-7-(2-(morpholinomethyl) acryloyl)-1,3,7-triazaspiro[4.5]decane-2,4-dione (Compound 635 and Compound 636)

To a stirred solution of 3-(4-(4-morpholino-7H-pyrrolo[2,3-d]pyrimidin-6-yl)phenyl)-1,3,7-triazaspiro[4.5]decane-2,4-dione 127 (200 mg, 0.447 mmol) and 2-(morpholinomethyl)acrylic acid 121 (115 mg, 0.670 mmol) in DMF (5 mL) were added DIPEA (0.25 mL, 1.34 mmol) and HATU (340 mg, 0.894 mmol) sequentially at 0° C. and the reaction mixture was stirred at RT for 2 h. After completion of reaction (monitoring by TLC), the reaction mixture was poured into ice cold water (20 mL) and extracted with 25% IPA in CHCl₃ (3×50 mL). The combined organic layer was dried over Na₂SO₄, filtered and concentrated under reduced pressure. The crude was purified by RP-HPLC [5-95% 5 mM ammonium bicarbonate in water/ACN; Column: X Select CSH C18 (19*250) 10μ] to get the Compound 635 (20 mg, 7%; Peak 1) and Compound 636 (14 mg, 5%; Peak 2).

Analytical data for Compound 635 (Peak 1): ¹H NMR (400 MHz, DMSO-d₆): δ 12.30 (s, 1H), 9.08 (s, 1H), 8.20 (s, 1H), 8.0 (d, J=8.4 Hz, 2H), 7.46 (d, J=8.4 Hz, 2H), 7.26 (s, 1H), 5.45-5.28 (m, 2H), 4.31-4.28 (m, 1H), 4.15-4.08 (m, 1H), 3.91-3.88 (m, 4H), 3.76-3.74 (m, 4H), 3.57 (brs, 4H), 3.23-3.18 (m, 2H), 3.11-3.05 (m, 2H), 2.40-2.35 (m, 4H), 2.07-2.02 (m, 1H), 1.92-1.89 (m, 1H), 1.76-1.70 (m, 2H).

LCMS=[M+H]⁺: 601.36; Purity=99.68%.

Analytical data for Compound 636 (Peak 2): ¹H NMR (400 MHz, DMSO-d₆): δ 12.30 (s, 1H), 8.87 (s, 1H), 8.19 (s, 1H), 7.99 (d, J=8.8 Hz, 2H), 7.43 (d, J=8.4 Hz, 2H), 7.25 (d, J=1.6 Hz, 1H), 5.53 (s, 1H), 5.21 (s, 1H), 3.91-3.88 (m, 4H), 3.76-3.74 (m, 4H), 3.58-3.52 (m, 8H), 3.23-3.20 (m, 1H), 3.08-3.05 (m, 1H), 2.85-2.79 (m, 2H), 2.36-2.44 (m, 1H), 2.11-2.05 (m, 1H), 1.84-1.66 (m, 4H).

LCMS=[M+H]⁺: 601.38; Purity=93.48%.

### Example 45

### Compound 803

### Preparation of (R)-4-(6-(4-(((4-((3-(1,1-dioxidoisothiazol-2 (3H)-yl)piperidin-1-yl)methyl)pyridin-2-yl)methyl)amino)phenyl)-7H-pyrrolo[2,3-d]pyrimidin-4-yl)thiomorpholine 1,1-dioxide (Compound 803)

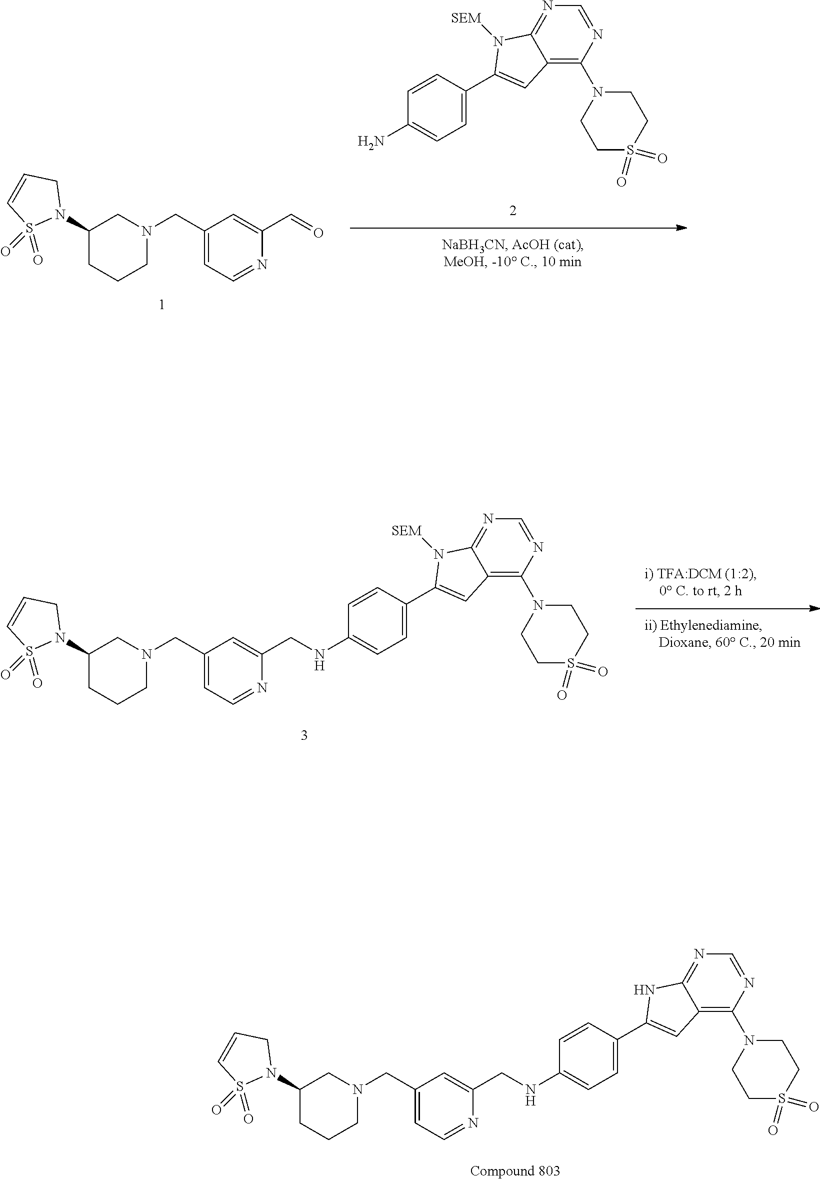 [SMILES: O=Cc1cc(CN2CCC[C@@H](N3CC=CS3(=O)=O)C2)ccn1 ; C[Si](C)(C)CCOCn1c(-c2ccc(N)cc2)cc2c(N3CCS(=O)(=O)CC3)ncnc21 ; C[Si](C)(C)CCOCn1c(-c2ccc(NCc3cc(CN4CCC[C@@H](N5CC=CS5(=O)=O)C4)ccn3)cc2)cc2c(N3CCS(=O)(=O)CC3)ncnc21 ; O=S1(=O)CCN(c2ncnc3[nH]c(-c4ccc(NCc5cc(CN6CCC[C@@H](N7CC=CS7(=O)=O)C6)ccn5)cc4)cc23)CC1]

### Synthesis of (R)-4-(6-(4-(((4-((3-(1,1-dioxidoisothiazol-2 (3H)-yl)piperidin-1-yl)methyl)pyridin-2-yl)methyl)amino)phenyl)-7-((2-(trimethylsilyl)ethoxy)methyl)-7H-pyrrolo[2,3-d]pyrimidin-4-yl)thiomorpholine 1,1-dioxide (3)

To a stirred solution of (R)-4-((3-(1,1-dioxidoisothiazol-2 (3H)-yl)piperidin-1-yl)methyl)picolinaldehyde 1 (1.02 g, 3.17 mmol) and 4-(6-(4-aminophenyl)-7-((2-(trimethylsilyl)ethoxy)methyl)-7H-pyrrolo[2,3-d]pyrimidin-4-yl) thiomorpholinc 1,1-dioxide 2 (1 g, 2.11 mmol) in MeOH (25 mL) was added AcOH (cat) at −10° C. under N₂ atmosphere and stirred 10 min at same temperature. Then NaCNBH₃ (253 mg, 4.22 mmol) was added to the reaction mixture at −10° C. and stirred for 10 min. After completion of reaction (TLC and LCMS monitoring), the reaction mixture was quenched with sat. NaHCO₃ (50 mL) and extracted with DCM (3×50 mL). The combined organic layer was dried over Na₂SO₄, filtered and concentrated under reduce pressure. The crude was purified by column chromatography (silica gel 100-200 M, 40 g SNAP) using eluent 2-3% McOH in DCM to get the title compound as brown solid 3 (1 g, 48%).

LCMS=[M+H]⁺: 779.5; Purity=83%.

### Synthesis of (R)-4-(6-(4-(((4-((3-(1,1-dioxidoisothiazol-2 (3H)-yl)piperidin-1-yl)methyl)pyridin-2-yl)methyl)amino)phenyl)-7H-pyrrolo[2,3-d]pyrimidin-4-yl)thiomorpholine 1,1-dioxide (Compound 803)

To a stirred solution of (R)-4-(6-(4-(((4-((3-(1,1-dioxidoisothiazol-2 (3H)-yl)piperidin-1-yl)methyl)pyridin-2-yl)methyl)amino)phenyl)-7-((2-(trimethylsilyl)ethoxy)methyl)-7H-pyrrolo[2,3-d]pyrimidin-4-yl)thiomorpholine 1,1-dioxide 3 (1 g, 1.28 mmol) in DCM (10 mL) was added TFA (5 mL) dropwise at 0° C. and the reaction mixture was stirred at RT for 2 h. After completion of SM (monitoring by TLC and LCMS), the reaction mixture was concentrated under reduce pressure. The crude was dissolved in 1,4-dioxane (10 mL) followed by 1,2-ethanediamine (0.13 mL, 1.93 mmol) was added to the reaction mixture and stirred at 60° C. for 20 min. After completion of reaction (monitoring by LCMS), the reaction mixture was poured into ice cold water (50 mL) and extracted with 25% IPA in CHCl₃ (3×50 mL). The combined organic layer was dried over Na₂SO₄, filtered and concentrated under reduced pressure. The crude was purified by RP-HPLC [5-95% 5 mM Ammonium acetate in H₂O/ACN; Column: X Bridge C18 (19*250) 10μ] to get Compound 803 (196 mg, 23%).

¹H NMR (400 MHz, DMSO-d₆): δ 12.05 (s, 1H), 8.46 (d, J=4.8 Hz, 1H), 8.18 (s, 1H), 7.62 (d, J=8.8 Hz, 2H), 7.32 (s, 1H), 7.18 (d, J=4.8 Hz, 1H), 7.09-7.07 (m, 2H), 6.82 (d, J=1.6 Hz, 1H), 6.64 (d, J=8.8 Hz, 2H), 6.60 (t, J=6.4 Hz, 1H), 4.39 (d, J=6 Hz, 2H), 4.30 (brs, 4H), 3.99-3.90 (m, 2H), 3.48-3.44 (m, 3H), 3.21 (brs, 4H), 2.87 (d, J=9.6 Hz, 1H), 2.53-2.49 (m, 1H), 2.16 (t, J=10.4 Hz, 1H), 1.90-1.81 (m, 2H), 1.63-1.60 (m, 1H), 1.50-1.45 (m, 2H).

LCMS=[M+H]⁺: 649.22; Purity=98.19%.

### Example 46

### Compound 640

### Preparation of 1-(4-(1-((4-(4-(3-oxa-8-azabicyclo[3.2.1]octan-8-yl)-7H-pyrrolo[2,3-d]pyrimidin-6-yl)phenyl)amino)-2,2,2-trifluoroethyl)piperidin-1-yl)-2-(azetidin-1-ylmethyl)prop-2-en-1-one (Compound 640)

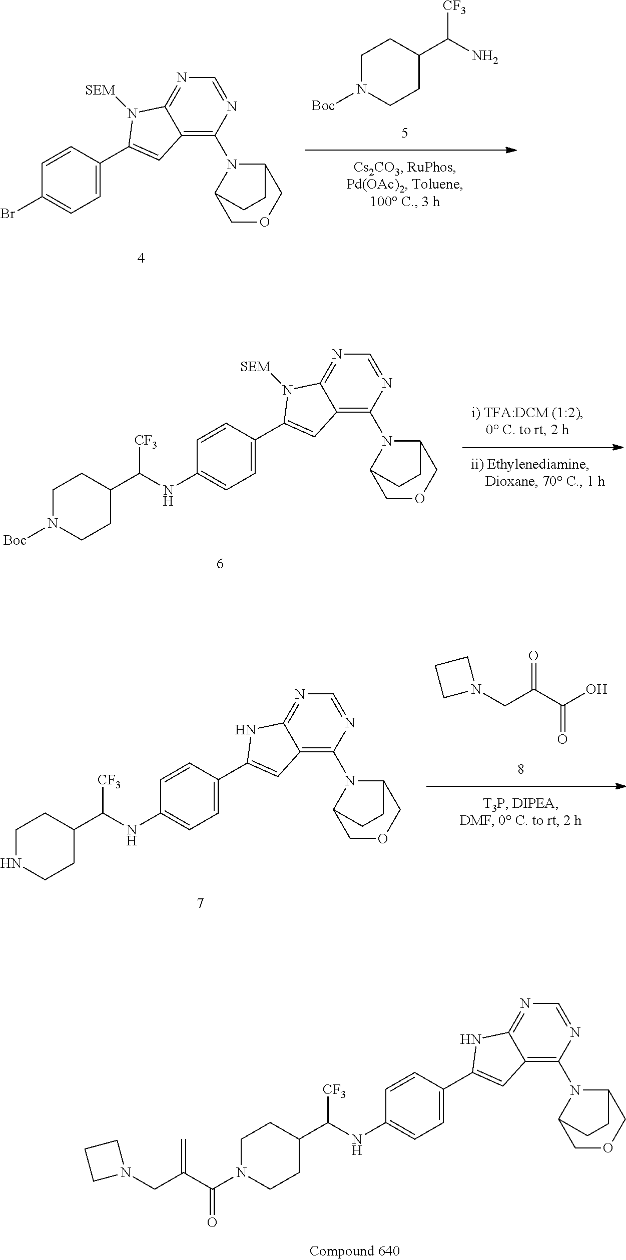 [SMILES: C[Si](C)(C)CCOCn1c(-c2ccc(Br)cc2)cc2c(N3C4CCC3COC4)ncnc21 ; CC(C)(C)OC(=O)N1CCC(C(N)C(F)(F)F)CC1 ; CC(C)(C)OC(=O)N1CCC(C(Nc2ccc(-c3cc4c(N5C6CCC5COC6)ncnc4n3COCC[Si](C)(C)C)cc2)C(F)(F)F)CC1 ; FC(F)(F)C(Nc1ccc(-c2cc3c(N4C5CCC4COC5)ncnc3[nH]2)cc1)C1CCNCC1 ; O=C(O)C(=O)CN1CCC1 ; C=C(CN1CCC1)C(=O)N1CCC(C(Nc2ccc(-c3cc4c(N5C6CCC5COC6)ncnc4[nH]3)cc2)C(F)(F)F)CC1]

### Synthesis of tert-butyl 4-(1-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)-2,2,2-trifluoroethyl)piperidine-1-carboxylate (6)

To a stirred solution of 8-(6-(4-bromophenyl)-7-((2-(trimethylsilyl)ethoxy)methyl)-7H-pyrrolo[2,3-d]pyrimidin-4-yl)-3-oxa-8-azabicyclo[3.2.1]octane 4 (1 g, 1.94 mmol) and tert-butyl 4-(1-amino-2,2,2-trifluoroethyl)piperidine-1-carboxylate 5 (767 mg, 2.72 mmol) in toluene (10 mL) was added Cs₂CO₃ (1.9 g, 5.82 mmol) at RT under N₂ atmosphere. The resulting reaction mixture was degassed with N₂ gas for 10 min, followed by RuPhos (181 mg, 0.388 mmol) and Pd(OAc)₂ (43.5 mg, 0.194 mmol) were added to the reaction mixture and stirred at 100° C. for 3 h. After completion of reaction (monitored by TLC), the reaction mixture was quenched with ice cold water (50 mL) and extracted with EtOAc (3×50 mL). The combined organic layer was washed with brine solution (2×20 mL), dried over Na₂SO₄, filtered and evaporated under reduced pressure. The crude was purified by flash chromatography (silica gel 100-200M, 40 g SNAP) using 65-70% EtOAc in heptane to obtain the desired product as pale yellow 6 (1.2 g, 86%).

LCMS=[M+H]⁺: 717.50; Purity=80%.

### Synthesis of 4-(4-(3-oxa-8-azabicyclo[3.2.1]octan-8-yl)-7H-pyrrolo[2,3-d]pyrimidin-6-yl)-N-(2,2,2-trifluoro-1-(piperidin-4-yl)ethyl) aniline (7)

To a stirred solution of tert-butyl 4-(1-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)-2,2,2-trifluoroethyl)piperidine-1-carboxylate 6 (1.2 g, 1.67 mmol) in DCM (10 mL) was added TFA (5 mL) dropwise at 0° C. and stirred at RT for 2 h. After completion of reaction (TLC monitoring), the reaction mixture was concentrated under reduced pressure. The crude was dissolved in 1,4-dioxane (10 mL), followed by 1,2-ethanediamine (0.22 mL, 3.28 mmol) was added to the reaction and stirred at 70° C. for 1 h. After completion of reaction (LCMS monitoring), the reaction mixture was diluted with 25% IPA in CHCl₃ (100 mL) and washed with ice cold water (2×50 mL). The organic layer was dried over Na₂SO₄, filtered and concentrated under reduced pressure to get the desired compound as an off white solid 7 (650 mg; crude) which was used as such for the next step without further purification.

LCMS=[M+H]⁺: 487.35; Purity=70%.

### Synthesis of (R)-4-(6-(4-(((4-((3-(1,1-dioxidoisothiazol-2 (3H)-yl)piperidin-1-yl)methyl)pyridin-2-yl)methyl)amino)phenyl)-7H-pyrrolo[2,3-d]pyrimidin-4-yl)thiomorpholine 1,1-dioxide (Compound 640)

To a stirred solution of 4-(4-(3-oxa-8-azabicyclo[3.2.1]octan-8-yl)-7H-pyrrolo[2,3-d]pyrimidin-6-yl)-N-(2,2,2-trifluoro-1-(piperidin-4-yl)ethyl) aniline 7 (200 mg, 0.411 mmol) in DMF (10 mL) were added 2-(azetidin-1-ylmethyl)acrylic acid 8 (87 mg, 0.617 mmol), DIPEA (0.21 mL, 1.23 mmol) and T3P (0.24 mL, 0.822 mmol; 50% in EtOAc) sequentially at 0° C. and stirred at RT for 2 h. After completion of reaction (LCMS monitoring), the reaction mixture was poured into ice cold water (50 mL) and extracted with EtOAc (3×50 mL). The combined organic layer was dried over Na₂SO₄, filtered and concentrated under reduced pressure. The crude was purified by RP-HPLC [5-95% 5 mM Ammonium bicarbonate in water/ACN; Column: X select Phenyl Hexyl (19*250) 5μ] to get Compound 640 (22 mg, 9%).

¹H NMR (400 MHz, DMSO-d₆): δ 11.94 (s, 1H), 8.12 (s, 1H), 7.65 (d, J=8.8 Hz, 2H), 6.87-6.84 (m, 3H), 6.10 (d, J=10 Hz, 1H), 5.22 (brs, 1H), 5.00-4.96 (m, 1H), 4.87 (brs, 2H), 4.38-4.36 (m, 2H), 3.91-3.85 (m, 1H), 3.69 (d, J=10.8 Hz, 2H), 3.59 (d, J=10.8 Hz, 2H), 3.08-3.04 (m, 7H), 2.52-2.49 (m, 1H), 2.17-1.94 (m, 7H), 1.77-1.74 (m, 2H), 1.41-1.38 (m, 2H).

LCMS=[M+H]⁺: 610.37; Purity=97.79%.

### Example 47

### Compound 641

### Preparation of tert-butyl(R)-(1-((2-((4-(4-chloro-7-((2-(trimethylsilyl)ethoxy)methyl)-7H-pyrrolo[2,3-d]pyrimidin-6-yl)phenyl)carbamoyl)pyridin-4-yl)methyl)piperidin-3-yl)carbamate (Compound 641)

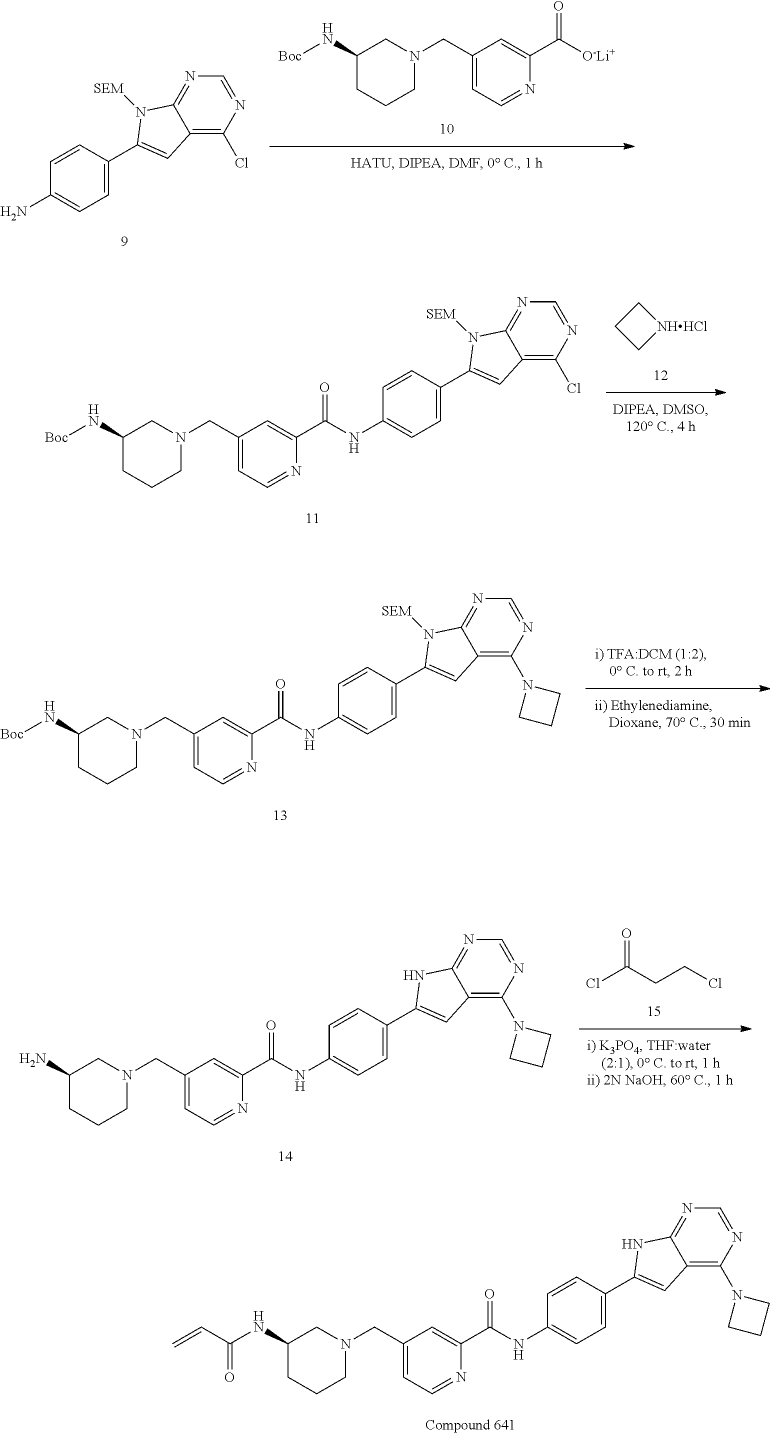

### Synthesis of tert-butyl(R)-(1-((2-((4-(4-chloro-7-((2-(trimethylsilyl)ethoxy)methyl)-7H-pyrrolo[2,3-d]pyrimidin-6-yl)phenyl)carbamoyl)pyridin-4-yl)methyl)piperidin-3-yl)carbamate (11)

To a stirred solution of 4-(4-chloro-7-((2-(trimethylsilyl)ethoxy)methyl)-7H-pyrrolo[2,3-d]pyrimidin-6-yl) aniline 9 (2.5 g, 6.67 mmol) in DMF (20 mL) were added lithium (R)-4-((3-((tert-butoxycarbonyl)amino)piperidin-1-yl)methyl)picolinate 10 (3.41 g, 10 mmol), DIPEA (3.45 mL, 20 mmol) and HATU (5.07 g, 13.3 mmol) sequentially at 0° C. and stirred at RT for 1 h. After completion of reaction (TLC monitoring), the reaction mass was poured into ice-cold water (50 mL) and extracted with EtOAc (3×100 mL). The combined organic layer was washed with ice cold brine solution (2×50 mL), dried over Na₂SO₄, filtered and evaporated under reduced pressure. The crude was purified by flash chromatography (silica gel 100-200 M, 40 g SNAP) using 15-20% EtOAc in heptane to get the title compound as a yellow solid 11 (3.5 g, 72%). ¹H NMR (400 MHz, DMSO-d₆): δ 10.90 (s, 1H), 8.71-8.69 (m, 2H), 8.14-8.12 (m, 3H), 7.84 (d, J=8.4 Hz, 2H), 7.62 (d, J=4.8 Hz, 1H), 6.90 (s, 1H), 6.78-6.73 (m, 1H), 5.66 (s, 2H), 3.65-3.62 (t, J=4.4 Hz, 2H), 3.45-3.40 (m, 1H), 2.88-2.72 (m, 3H), 1.92-1.84 (m, 2H), 1.72-1.66 (m, 2H), 1.49-1.47 (m, 1H), 1.45 (s, 9H), 1.24-1.19 (m, 2H), 0.86 (t, J=8 Hz, 2H), −0.08 (s, 9H).

LCMS=[M+H]⁺: 692.56; Purity=95%.

### Synthesis of tert-butyl(R)-(1-((2-((4-(4-(azetidin-1-yl)-7-((2-(trimethylsilyl)ethoxy)methyl)-7H-pyrrolo[2,3-d]pyrimidin-6-yl)phenyl)carbamoyl)pyridin-4-yl)methyl)piperidin-3-yl)carbamate (13)

To a stirred solution of tert-butyl(R)-(1-((2-((4-(4-chloro-7-((2-(trimethylsilyl)ethoxy)methyl)-7H-pyrrolo[2,3-d]pyrimidin-6-yl)phenyl)carbamoyl)pyridin-4-yl)methyl)piperidin-3-yl)carbamate 11 (800 mg, 1.16 mmol) in DMSO (10 mL) were added DIPEA (0.6 mL, 3.47 mmol) and azetidine hydrochloride 12 (162 mg, 1.73 mmol) sequentially at RT and stirred at 120° C. for 4 h. After completion of reaction (TLC monitoring), the reaction mixture was cooled to RT, poured into ice cold-water (50 mL) and extracted with EtOAc (2×50 mL). The combined organic layer was washed with ice cold brine (2×50 mL), dried over Na₂SO₄, filtered and evaporated under reduced pressure to get the title compound as a yellow solid 13 (700 mg, 73%). LCMS=[M+H]⁺: 713.66; Purity=91%.

### Synthesis of (R)-4-((3-aminopiperidin-1-yl)methyl)-N-(4-(4-(azetidin-1-yl)-7H-pyrrolo[2,3-d]pyrimidin-6-yl)phenyl)picolinamide (14)

To a stirred solution of tert-butyl(R)-(1-((2-((4-(4-(azetidin-1-yl)-7-((2-(trimethylsilyl)ethoxy)methyl)-7H-pyrrolo[2,3-d]pyrimidin-6-yl)phenyl)carbamoyl)pyridin-4 -yl)methyl)piperidin-3-yl)carbamate 13 (600 mg, 0.842 mmol) in DCM (6 mL) was added TFA (3 mL) dropwise at 0° C. under N₂ atmosphere and stirred at RT for 2 h. After completion of reaction (TLC monitoring), the reaction mixture was concentrated under reduced pressure. The crude was dissolved in 1,4-dioxane (10 mL), followed by 1,2-ethanediamine (0.12 mL, 1.68 mmol) was added to the reaction mixture and stirred at 70° C. for 30 min. After completion of reaction (LCMS monitoring), the reaction mixture was poured into ice cold water (50 mL) and extracted with 25% IPA in CHCl₃ (3×50 mL). The combined organic layer was dried over Na₂SO₄, filtered and concentrated under reduced pressure to get the title compound as a yellow solid 14 (350 mg; crude) which was used in next step directly without further purification.

LCMS=[M+H]⁺: 483.44; Purity=93%.

### Synthesis of (R)-4-((3-acrylamidopiperidin-1-yl)methyl)-N-(4-(4-(azetidin-1-yl)-7H-pyrrolo[2,3-d]pyrimidin-6-yl)phenyl)picolinamide (Compound 641)

To a stirred solution of (R)-4-((3-aminopiperidin-1-yl)methyl)-N-(4-(4-(azetidin-1-yl)-7H-pyrrolo[2,3-d]pyrimidin-6-yl)phenyl)picolinamide 14 (360 mg, 0.746 mmol) in THF (10 mL) was added dissolved in water 5 mL at 0° C. Then, 3-Chloropropionyl chloride 15 (17.8 mg, 0.140 mmol) dissolved in THF (1 mL) was added dropwise to the reaction mixture at 0° C. and stirred at RT for 1 h. After completion of reaction (TLC monitoring), 2N aq. NaOH (4.48 mL, 8.95 mmol) was added to the reaction mixture and stirred at 60° C. for 1 h. After the completion of reaction (LCMS monitoring), the reaction mixture was diluted with water (50 mL) and extracted with 25% IPA in CHCl₃ (3×20 mL). The combined organic was dried over Na₂SO₄, filtered and concentrated under reduced pressure. The crude was purified by RP-HPLC [5-95% 0.1% TFA in H₂O/ACN; Column: SunFire C18 (19*250) 10μ] to get Compound 641 (239 mg, 59%).

¹H NMR (400 MHz, DMSO-d₆): δ 12.96 (brs, 1H), 10.87 (s, 1H), 8.85 (d, J=4.0 Hz, 1H), 8.36 (s, 1H), 8.28 (s, 2H), 8.04 (d, J=8.8 Hz, 2H) 7.92 (d, J=8.8 Hz, 2H), 7.78 (brs, 1H), 7.15 (s, 1H), 6.23-6.17 (m, 1H), 6.12-6.08 (m, 1H), 5.63 (d, J=11.6 Hz, 1H), 4.53 (brs, 6H), 4.02 (brs, 1H), 2.95-2.93 (m, 1H), 2.58-2.55 (m, 2H), 1.92-1.84 (m, 2H), 1.70-1.68 (m, 1H), 1.42-1.39 (m, 1H). [3 protons are may merged in solvent peak]

LCMS=[M+H]⁺: 537.28; Purity=99.47%.

### Example 48

### Compound 642

### Preparation of (R)-4-((3-acrylamidopiperidin-1-yl)methyl)-N-(4-(4-(3-hydroxyazetidin-1-yl)-7H-pyrrolo[2,3-d]pyrimidin-6-yl)phenyl)picolinamide (Compound 642)

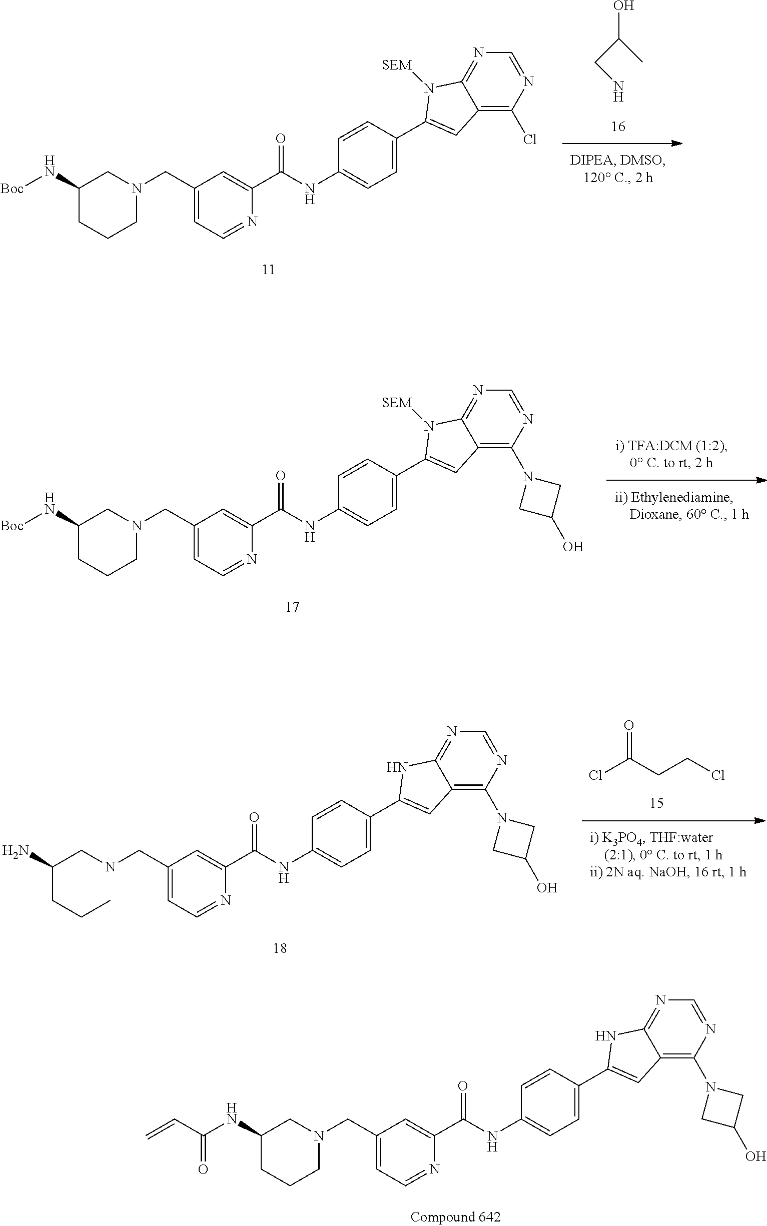

### Synthesis of tert-butyl(R)-(1-((2-((4-(4-(3-hydroxyazetidin-1-yl)-7-((2-(trimethylsilyl)ethoxy)methyl)-7H-pyrrolo[2,3-d]pyrimidin-6-yl)phenyl)carbamoyl)pyridin-4-yl)methyl)piperidin-3-yl)carbamate (17)

To a stirred solution of tert-butyl(R)-(1-((2-((4-(4-chloro-7-((2-(trimethylsilyl)ethoxy)methyl)-7H-pyrrolo[2,3-d]pyrimidin-6-yl)phenyl)carbamoyl)pyridin-4-yl)methyl)piperidin-3-yl)carbamate 11 (800 mg, 1.16 mmol) in DMSO (10 mL) were added DIPEA (0.6 mL, 3.47 mmol) and 3-azetidinol 17 (127 mg, 1.73 mmol) sequentially at RT. The resulting reaction mixture was stirred at 120° C. for 2 h. After completion of reaction (TLC monitoring), the reaction mixture was cooled to RT, poured into ice cold-water (50 mL) and extracted with EtOAc (3×50 mL). The combined organic layer was washed with brine (2×50 mL), dried over Na₂SO₄, filtered and evaporated under reduced pressure to get title compound as an off white solid 17 (800 mg), which was forwarded to next step without further purification.

LCMS: [M+H]⁺: 729.53; Purity=80%.

### Synthesis of (R)-4-((3-aminopiperidin-1-yl)methyl)-N-(4-(4-(3-hydroxyazetidin-1-yl)-7H-pyrrolo[2,3-d]pyrimidin-6-yl)phenyl)picolinamide (18)

To a stirred solution of tert-butyl(R)-(1-((2-((4-(4-(3-hydroxyazetidin-1-yl)-7-((2-(trimethylsilyl)ethoxy)methyl)-7H-pyrrolo[2,3-d]pyrimidin-6-yl)phenyl)carbamoyl)pyridin-4-yl)methyl)piperidin-3-yl)carbamate 17 (800 mg, 1.1 mmol) in DCM (8 mL) was added TFA (4 mL) dropwise at 0° C. and stirred at RT for 2 h. After completion of reaction (TLC and LCMS monitoring), the reaction mixture was concentrated under reduced pressure, triturated with Et₂₀ and dried under reduced pressure. The crude was dissolved in 1,4-dioxane (10 mL), followed by 1,2-ethanediamine (0.11 mL, 1.65 mmol) was added to the reaction mixture and stirred at 60° C. for 1 h. After completion of reaction (monitoring by LCMS), the reaction was diluted with water (50 mL) and extracted with 25% IPA in CHCl₃ (3×50 mL), dried over Na₂SO₄, filtered and concentrated under reduced pressure to get the title compound as a yellow semi solid 18 (350 mg; crude) which was used in next step directly without further purification LCMS=[M+H]⁺: 499.24; Purity=80%.

### Synthesis of (R)-4-((3-acrylamidopiperidin-1-yl)methyl)-N-(4-(4-(3-hydroxyazetidin-1-yl)-7H-pyrrolo[2,3-d]pyrimidin-6-yl)phenyl)picolinamide (Compound 642)

To a stirred solution of (R)-4-((3-aminopiperidin-1-yl)methyl)-N-(4-(4-(3-hydroxyazetidin-1-yl)-7H-pyrrolo[2,3-d]pyrimidin-6-yl)phenyl)picolinamide 18 (320 mg, 0.642 mmol) in THF (6 mL) was added K3PO₄ (272 mg, 1.28 mmol) dissolved in water (3 mL) at 0° C. Then, 3-chloropropionyl chloride 15 (73.5 μL, 0.770 mmol) dissolved in THF (1 mL) was added dropwise to the reaction mixture at 0° C. and stirred at RT for resulting reaction mixture was allowed to stir at RT for 1 h. After completion of SM (monitoring by TLC), 2N aq. NaOH (3.8 mL, 6.16 mmol) was added to the reaction mixture and stirred at RT for 1 h. After the completion of reaction (TLC and LCMS monitoring), the reaction mixture was diluted with water (50 mL) and extracted with 25% IPA in CHCl₃ (3×50 mL). The combined organic was dried over Na₂SO₄, filtered and concentrated under reduce pressure. The crude was purified by RP-HPLC [10-90% 0.1% TFA in H₂O/ACN; Column: Acquity HSS-T3 (2.1×100 mm) 1.8 μm] to get Compound 642 (130 mg, 37%). ¹H NMR (400 MHz, DMSO-d₆): δ 12.10 (s, 1H), 10.74 (s, 1H), 8.68 (d, J=4.8 Hz, 1H), 8.11 (s, 2H), 7.99 (d, J=8.4 Hz, 3H), 7.87 (d, J=8.8 Hz, 2H), 7.62 (d, J=4 Hz, 1H), 6.88 (d, J=2 Hz, 1H), 6.25-6.18 (m, 1H), 6.05 (dd, J=16.8 Hz, 2.2 Hz, 1H), 5.80 (d, J=6.8 Hz, 1H), 5.55 (dd, J=10.4 Hz, 2.4 Hz, 1H), 4.69-4.64 (m, 1H), 4.55-4.49 (m, 2H), 4.06-3.99 (m, 2H), 3.83-3.82 (m, 1H), 3.66 (s, 2H), 2.79-2.77 (m, 1H), 2.64-2.60 (m, 1H), 2.06-2.01 (m, 1H), 1.90 (t, J=10.4 Hz, 1H), 1.77-1.68 (m, 2H), 1.55-1.51 (m, 1H), 1.23-1.17 (m, 1H). LCMS=[M+H]⁺: 553.35; Purity=95.52%.

### Example 49

### Compound 643

### Preparation of (R)-4-((3-acrylamidopiperidin-1-yl)methyl)-N-(4-(4-(pyrrolidin-1-yl)-7H-pyrrolo[2,3-d]pyrimidin-6-yl)phenyl)picolinamide (Compound 643)

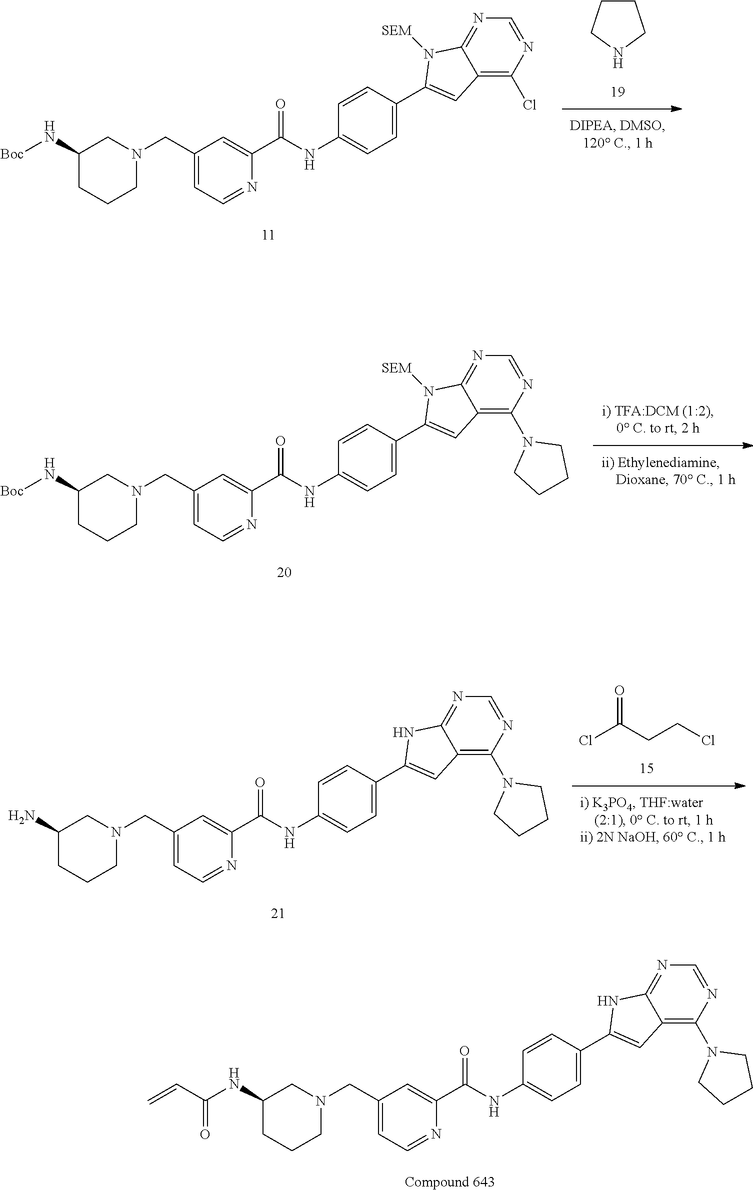 [SMILES: CC(C)(C)OC(=O)N[C@@H]1CCCN(Cc2ccnc(C(=O)Nc3ccc(-c4cc5c(Cl)ncnc5n4COCC[Si](C)(C)C)cc3)c2)C1 ; C1CCNC1 ; CC(C)(C)OC(=O)N[C@@H]1CCCN(Cc2ccnc(C(=O)Nc3ccc(-c4cc5c(N6CCCC6)ncnc5n4COCC[Si](C)(C)C)cc3)c2)C1 ; N[C@@H]1CCCN(Cc2ccnc(C(=O)Nc3ccc(-c4cc5c(N6CCCC6)ncnc5[nH]4)cc3)c2)C1 ; O=C(Cl)CCCl ; C=CC(=O)N[C@@H]1CCCN(Cc2ccnc(C(=O)Nc3ccc(-c4cc5c(N6CCCC6)ncnc5[nH]4)cc3)c2)C1]

### Synthesis of tert-butyl(R)-(1-((2-((4-(4-(pyrrolidin-1-yl)-7-((2-(trimethylsilyl)ethoxy)methyl)-7H-pyrrolo[2,3-d]pyrimidin-6-yl)phenyl)carbamoyl)pyridin-4-yl)methyl)piperidin-3-yl)carbamate (20)

To a stirred solution of tert-butyl(R)-(1-((2-((4-(4-chloro-7-((2-(trimethylsilyl)ethoxy)methyl)-7H-pyrrolo[2,3-d]pyrimidin-6-yl)phenyl)carbamoyl)pyridin-4-yl)methyl)piperidin-3-yl)carbamate 11 (800 mg, 1.16 mmol) in DMSO (10 mL) were added DIPEA (0.60 mL, 3.47 mmol) and pyrrolidine 19 (0.12 mL, 1.73 mmol) sequentially at RT. The resulting reaction mixture was stirred at 120° C. for 4 h. After completion of reaction (TLC monitoring), the reaction mixture was cooled to RT, poured into ice cold water (50 mL), and extracted with EtOAc (2×50 mL). The combined organic layer was washed with brine (3×50 mL), dried over Na₂SO₄, filtered and evaporated under reduced pressure. The crude was purified by flash chromatography (silica gel 100-200 M, 12 g SNAP) using 15-20% EtOAc in heptane to get the title compound as sticky brown liquid 20 (800 mg, 95%).

LCMS=[M+H]⁺: 727.58; Purity=98.36%.

### Synthesis of (R)-4-((3-aminopiperidin-1-yl)methyl)-N-(4-(4-(pyrrolidin-1-yl)-7H-pyrrolo[2,3-d]pyrimidin-6-yl)phenyl)picolinamide (21)

To a stirred solution of tert-butyl(R)-(1-((2-((4-(4-(pyrrolidin-1-yl)-7-((2-(trimethylsilyl)ethoxy)methyl)-7H-pyrrolo[2,3-d]pyrimidin-6-yl)phenyl)carbamoyl)pyridin-4-yl)methyl)piperidin-3-yl)carbamate 20 (1 g, 1.38 mmol) in DCM (10 mL) was added TFA (5 mL) dropwise at 0° C. and stirred at RT for 2 h. After completion of reaction (TLC monitoring), the reaction mixture was concentrated under reduced pressure. The crude was dissolved in 1,4-dioxane (10 mL), followed by 1,2 -ethanediamine (0.14 mL, 2.06 mmol) was added to the reaction mixture and stirred at 70° C. for 1 h. After completion of reaction (LCMS monitoring), the reaction mixture was diluted with water (50 mL) and extracted with 25% IPA in CHCl₃ (3×50 mL). The combined organic layer was dried over Na₂SO₄, filtered and concentrated under reduced pressure to get the title compound as a yellow solid 21 (650 mg; crude) which was used in next step directly without further purification.

LCMS=[M+H]⁺: 497.35; Purity=88%.

### Synthesis of (R)-4-((3-acrylamidopiperidin-1-yl)methyl)-N-(4-(4-(pyrrolidin-1-yl)-7H-pyrrolo[2,3-d]pyrimidin-6-yl)phenyl)picolinamide (Compound 643)

To a stirred solution of (R)-4-((3-aminopiperidin-1-yl)methyl)-N-(4-(4-(pyrrolidin-1-yl)-7H-pyrrolo[2,3-d]pyrimidin-6-yl)phenyl)picolinamide 21 (600 mg, 1.21 mmol) in THF (6 mL) was added K3PO₄ (513 mg, 2.42 mmol) dissolved in water (3 mL) at 0° C. Then, 3-chloropropionyl chloride 15 (184 mg, 1.45 mmol) was added dropwise to the reaction mixture and stirred at RT for 1 h. After consumed of SM (monitoring by TLC), 2M aq. NaOH (7.2 mL, 14.5 mmol) was added dropwise at 0° C. and stirred at 60° C. for 1 h. After completion of reaction (LCMS monitoring), the reaction mixture was poured into ice cold water (50 mL) and extracted with 2-MeTHF (3×50 mL). The combined organic layer was dried over Na₂SO₄, filtered and concentrated under reduced pressure. The crude was purified by RP-HPLC [5-95% 5 mM ammonium bicarbonate in H₂O/ACN; Column: X bridge sheild RP18 (19*250) 10μ] to get Compound 643 (190 mg, 28%). ¹H NMR (400 MHz, DMSO-d₆): δ 12.03 (s, 1H), 10.72 (s, 1H), 8.68 (d, J=5.2 Hz, 1H), 8.11-8.09 (m, 2H), 7.99-7.96 (m, 3H), 7.88-7.86 (m, 2H), 7.62 (d, J=4.8 Hz, 1H), 7.06 (d, J=2 Hz, 1H), 6.25-6.18 (m, 1H), 6.05 (dd, J=17.2 Hz, 2.4 Hz, 1H), 5.55 (dd, J=10.4 Hz, 2 Hz, 1H), 3.88-3.78 (m, 5H), 3.66 (s, 2H), 2.79-2.77 (m, 1H), 2.66-2.64 (m, 1H), 2.06-1.99 (m, 5H), 1.90 (t, J=9.6 Hz, 1H), 1.77-1.68 (m, 2H), 1.57-1.52 (m, 1H), 1.24-1.19 (m, 1H).

LCMS=[M+H]⁺: 551.29; Purity=99.66%.

### Example 50

### Compound 644

### Preparation of N-(4-(4-((1R,5S)-3-oxa-8-azabicyclo[3.2.1]octan-8-yl)-7H-pyrrolo[2,3-d]pyrimidin-6-yl)phenyl)-4-(((R)-3-(2-((3-fluoroazetidin-1-yl)methyl)acrylamido)piperidin-1-yl)methyl)picolinamide (Compound 644)

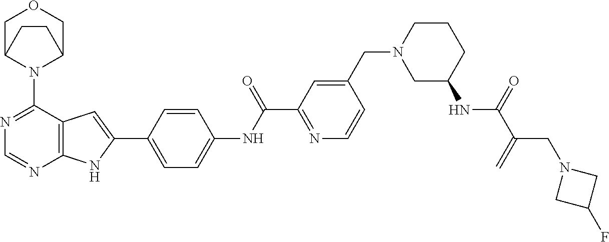 [SMILES: C=C(CN1CC(F)C1)C(=O)N[C@@H]1CCCN(Cc2ccnc(C(=O)Nc3ccc(-c4cc5c(N6C7CCC6COC7)ncnc5[nH]4)cc3)c2)C1]

Step 1:2-((3-Fluoroazetidin-1-yl)methyl)acrylic acid

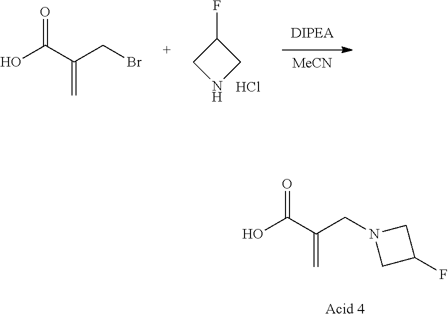

A solution of 2-(bromomethyl)prop-2-enoic acid (250 mg, 1.51 mmol), 3-fluoroazetidine hydrochloride (219 mg, 1.96 mmol), and N,N-diisopropylethylamine (0.79 mL, 4.54 mmol) in acetonitrile (4 mL) was stirred at 35° C. for 1 hour, providing a solution of 2-((3-fluoroazetidin-1-yl)methyl)acrylic acid (241 mg, 1.51 mmol, 99%) in acetonitrile. The reaction was assumed to be quantitative. Aliquots of this solution were used in the next step without workup.

Step 2: N-(4-(4-((1R,5S)-3-oxa-8-azabicyclo[3.2.1]octan-8-yl)-7H-pyrrolo[2,3-d]pyrimidin-6-yl)phenyl)-4-(((R)-3-(2-((3-fluoroazetidin-1-yl)methyl)acrylamido)piperidin-1-yl)methyl)picolinamide (Compound 644)

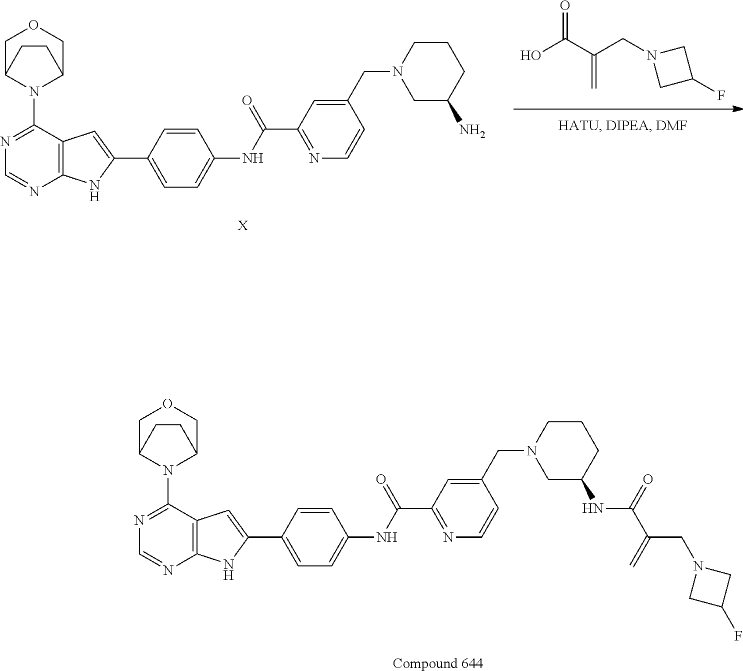

N-(4-(4-((1R,5S)-3-oxa-8-azabicyclo[3.2.1]octan-8-yl)-7H-pyrrolo[2,3-d]pyrimidin-6-yl)phenyl)-4-(((R)-3-aminopiperidin-1-yl)methyl)picolinamide X (10 mg, 0.0186 mmol) was added to a solution of HATU (13 mg, 0.0334 mmol), 2-((3-fluoroazetidin-1-yl)methyl)acrylic acid (5.3 mg, 0.0334 mmol) (as a solution in acetonitrile), and N,N-diisopropylethylamine (0.0025 mL, 0.149 mmol) in N,N-dimethylformamide (0.2 mL). After 3 hours the reaction was subjected to preparative HPLC(5-85% acetonitrile/water, 0.1% trifluoroacetic acid). The fractions containing product were combined and subjected to lyophilization, providing Compound 644 (2.1 mg, 0.0259 mmol, 14.7%). ¹H NMR (400 MHz, MeOD) δ 8.87 (d, J=4.9 Hz, 1H), 8.42 (s, 1H), 8.29 (s, 1H), 8.01 (d, J=8.7 Hz, 2H), 7.93 (d, J=8.7 Hz, 2H), 7.79 (d, J=4.9 Hz, 1H), 7.33 (s, 1H), 6.28 (s, 1H), 6.08 (s, 1H), 5.45 (s, 1H), 5.31 (s, 1H), 5.06 (b, 1H), 4.54 (m, 4H), 4.34 (m, 2H), 4.22 (m, 1H), 4.12 (s, 2H), 3.94-3.83 (m, 4H), 3.53 (m, 2H), 3.02 (m, 1H), 2.41-2.18 (m, 4H), 2.07 (m, 2H), 1.92 (m, 1H), 1.72 (m, 1H).

LCMS=[M+H]⁺: 680.4; Purity=96.6%.

### Example 51

### Compound 645

### Preparation of (R)-4-((3-(2-((methyl (2,2,2-trifluoroethyl)amino)methyl)acrylamido)piperidin-1-yl)methyl)-N-(4-(4-morpholino-7H-pyrrolo[2,3-d]pyrimidin-6-yl)phenyl)picolinamide (Compound 645)

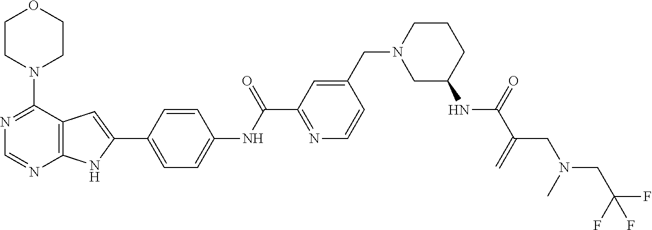 [SMILES: C=C(CN(C)CC(F)(F)F)C(=O)N[C@@H]1CCCN(Cc2ccnc(C(=O)Nc3ccc(-c4cc5c(N6CCOCC6)ncnc5[nH]4)cc3)c2)C1]

Step 1:2-((Methyl (2,2,2-trifluoroethyl)amino)methyl)acrylic acid

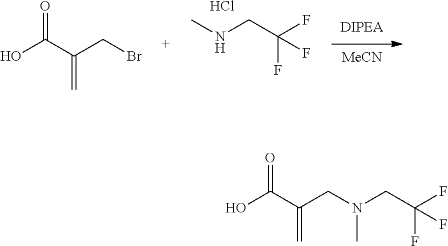

A solution of 2-(bromomethyl)prop-2-enoic acid (250 mg, 1.52 mmol), 2,2,2-trifluoro-N-methyl-ethanamine hydrochloride (295 mg, 1.97 mmol), and N,N-diisopropylethylamine (0.79 mL, 4.55 mmol) in acetonitrile (4 mL) was stirred at 35° C. for 20 hours, providing a solution of 2-((3,3-difluoroazetidin-1-yl)methyl)acrylic acid (298 mg, 1.51 mmol, 99%) in acetonitrile. The reaction was assumed to be quantitative. Aliquots of this solution were used in the next step without workup.

Step 2: (R)-4-((3-(2-((Methyl (2,2,2-trifluoroethyl)amino)methyl)acrylamido)piperidin-1-yl)methyl)-N-(4-(4-morpholino-7H-pyrrolo[2,3-d]pyrimidin-6-yl)phenyl)picolinamide (Compound 645)

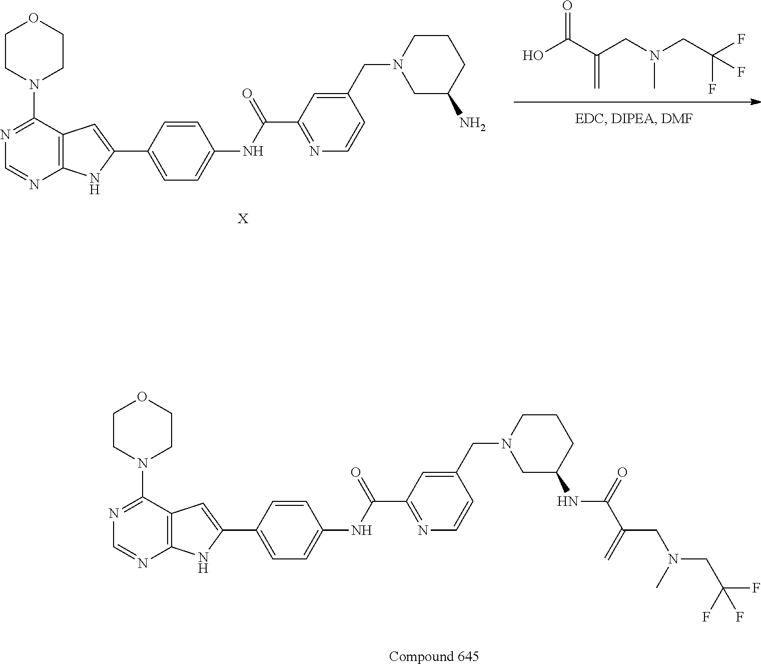

(R)-4-((3-Aminopiperidin-1-yl)methyl)-N-(4-(4-morpholino-7H-pyrrolo[2,3-d]pyrimidin-6-yl)phenyl)picolinamide trihydrochloride X (50 mg, 0.080 mmol) was added to a solution of 1-(3-dimethylaminopropyl)-3-ethylcarbodiimide hydrochloride (23 mg, 0.121 mmol), 2-((methyl (2,2,2-trifluoroethyl)amino)methyl)acrylic acid (24 mg, 0.121 mmol) (as a solution in acetonitrile), and N,N-diisopropylethylamine (0.112 mL, 0.643 mmol) in N,N-dimethylformamide (2 mL). After 16 hours the mixture was subjected to preparative HPLC(5-85% acetonitrile/water, 0.1% trifluoroacetic acid). The fractions containing product were combined and subjected to lyophilization, providing of Compound 645 (29 mg, 0.0338 mmol, 42%). ¹H NMR (400 MHz, MeOD) & 8.76 (d, J=4.9 Hz, 1H), 8.33-8.28 (m, 1H), 8.21 (s, 1H), 7.93-7.85 (m, 2H), 7.83-7.75 (m, 2H), 7.66 (dd, J=4.9, 1.7 Hz, 1H), 7.20 (s, 1H), 5.92 (s, 1H), 5.49 (d, J=1.3 Hz, 1H), 4.48-4.36 (m, 2H), 4.10-3.93 (m, 5H), 3.83 (t, J=4.9 Hz, 4H), 3.46 (m, 1H), 3.42-3.28 (m, 3H), 3.04 (m, 3H), 2.84-2.58 (m, 1H), 2.31 (s, 3H), 1.95 (m, 2H), 1.79 (m, 1H), 1.51 (m, 1H).

LCMS=[M+H]⁺: 679.40; Purity=94%.

### Example 52

### Compound 646

### Preparation of 4-[[(3R)-3-[2-[(3-fluoro-3-methyl-azetidin-1-yl)methyl]prop-2-enoylamino]-1-piperidyl]methyl]-N-[4-[4-(3-oxa-8-azabicyclo[3.2.1]octan-8-yl)-7H-pyrrolo[2,3-d]pyrimidin-6-yl]phenyl]pyridine-2-carboxamide; 2,2,2-trifluoroacetate (Compound 646)

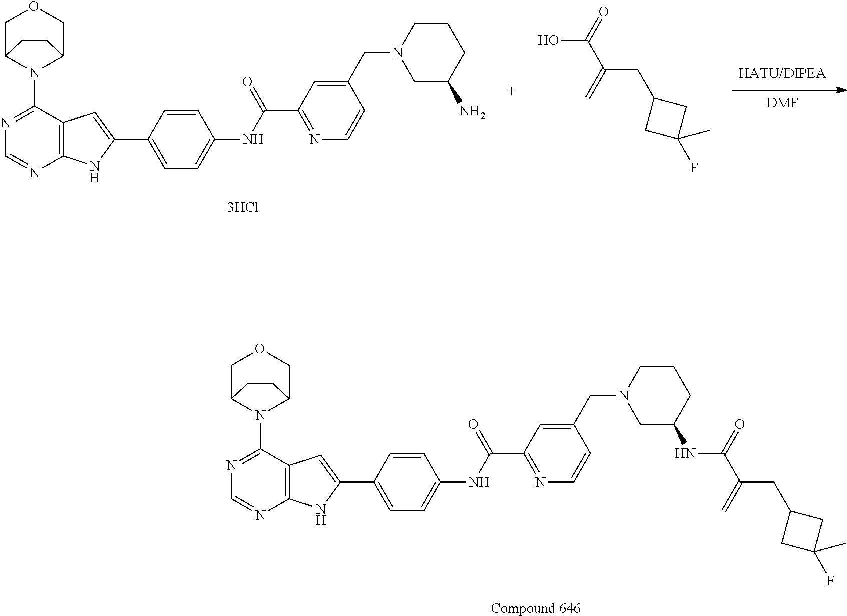

To a solution of 2-[(3-fluoro-3-methyl-azetidin-1-yl)methyl]prop-2-enoic acid (4.8 mg, 0.0278 mmol, 1.80 eq) in DMF (0.16 mL) was added O-(7-azabenzotriazol-1-yl)-N,N,N′,N′-tetramethyuluranium hexafluorophosphate (10 mg, 0.0276 mmol, 1.79 eq) followed by N,N-diisopropylethylamine (0.019 mL, 0.108 mmol, 6.99 eq) and 4-[[(3R)-3-amino-1-piperidyl]methyl]-N-[4-[4-(3-oxa-8-azabicyclo[3.2.1]octan-8-yl)-7H-pyrrolo[2,3-d]pyrimidin-6-yl]phenyl]pyridine-2-carboxamide; trihydrochloride (10 mg, 0.0154 mmol, 1.00 eq). The reaction was stirring at room temperature for 30 minutes. LCMS showed desire product. Water and ethyl acetate was added to the reaction mixture. The combined organic layers were washed with brine. The organic layers were concentrated and was submit to HPLC purification eluting 2% acetonitrile/water to 60% acetonitrile/water 30 minutes. The product fractions were collected and lyophilized to give as 4-[[(3R)-3-[2-[(3-fluoro-3-methyl-azetidin-1-yl)methyl]prop-2-enoylamino]-1-piperidyl]methyl]-N-[4-[4-(3-oxa-8 -azabicyclo[3.2.1]octan-8-yl)-7H-pyrrolo[2,3-d]pyrimidin-6-yl]phenyl]pyridine-2-carboxamide; 2,2,2-trifluoroacetate (1.1 mg, 0.00135 mmol, 8.75% yield).

### Example 53

### Compound 901

### N-(4-(4-((1R,5S)-3-oxa-8-azabicyclo[3.2.1]octan-8-yl)-7H-pyrrolo[2,3-d]pyrimidin-6-yl)phenyl)-4-(((R)-3-(2-fluoroacrylamido)piperidin-1-yl)methyl)picolinamide (Compound 901)

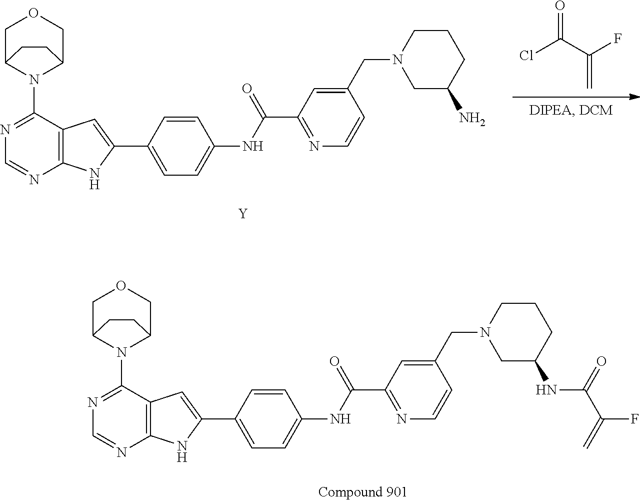

2-Fluoroprop-2-enoyl chloride (4.5 mg, 0.0418 mmol) was added to a solution of N,N-diisopropylethylamine (0.039 mL, 0.223 mmol) and N-(4-(4-((1R,5S)-3-oxa-8-azabicyclo[3.2.1]octan-8-yl)-7H-pyrrolo[2,3-d]pyrimidin-6-yl)phenyl)-4-(((R)-3-aminopiperidin-1-yl)methyl)picolinamide (15 mg, 0.0278 mmol) in dichloromethane (3 mL) and stirred at 22° C. After 45 minutes N,N-dimethylformamide (5 mL) was added, and the dichloromethane was removed under reduced pressure. The solution was subjected to preparative HPLC(5-85% acetonitrile/water, 0.1% trifluoroacetic acid). The fractions containing product were combined and subjected to lyophilization, providing Compound 901 (9.6 mg, 0.0126 mmol, 45% yield). ¹H NMR (400 MHz, MeOD) & 8.76 (d, J=4.9 Hz, 1H), 8.32-8.27 (m, 1H), 8.16 (s, 1H), 7.92-7.85 (m, 2H), 7.84-7.76 (m, 2H), 7.66 (dd, J=4.9, 1.7 Hz, 1H), 7.19 (s, 1H), 5.50 (dd, J=46.6, 3.5 Hz, 1H), 5.13 (dd, J=15.2, 3.5 Hz, 1H), 4.94 (m, 2H), 4.41 (s, 2H), 4.12 (m, 1H), 3.78 (m, 4H), 3.43 (d, J=12.3 Hz, 1H), 3.38 (s, 1H), 3.06-2.61 (m, 2H), 2.28-2.05 (m, 4H), 2.05-1.87 (m, 2H), 1.78 (m, 1H), 1.59 (m, 1H). LCMS=[M+H]⁺: 611.4; Purity=95%.

### Example 54

### Compound 902

### (R)—N-(4-(4-morpholino-7H-pyrrolo[2,3-d]pyrimidin-6-yl)phenyl)-4-((3-(2-((3-(trifluoromethyl)azetidin-1-yl)methyl)acrylamido)piperidin-1-yl)methyl)picolinamide (Compound 902)

Step 1:2-((3-(trifluoromethyl)azetidin-1-yl)methyl)acrylic acid

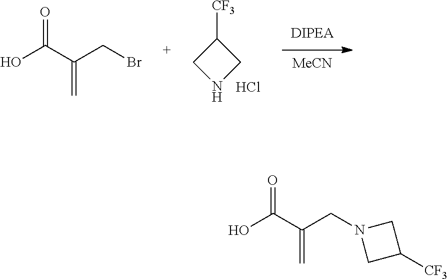

A solution of 2-(bromomethyl)prop-2-enoic acid (50 mg, 0.303 mmol), 3-(trifluoromethyl)azetidine hydrochloride (64 mg, 0.394 mmol), and N,N-diisopropylethylamine (0.16 mL, 0.909 mmol) in acetonitrile (2 mL) was stirred at 35° C. for 20 hours, providing a solution of 2-((3-(trifluoromethyl)azetidin-1-yl)methyl)acrylic acid (63 mg, 0.301 mmol, 99%) in acetonitrile. The solution was used in the next step without workup.

LCMS=[M+H]⁺: 210.1

Step 2: (R)—N-(4-(4-morpholino-7H-pyrrolo[2,3-d]pyrimidin-6-yl)phenyl)-4-((3-(2-((3-(trifluoromethyl)azetidin-1-yl)methyl)acrylamido)piperidin-1-yl)methyl)picolinamide (Compound 902)

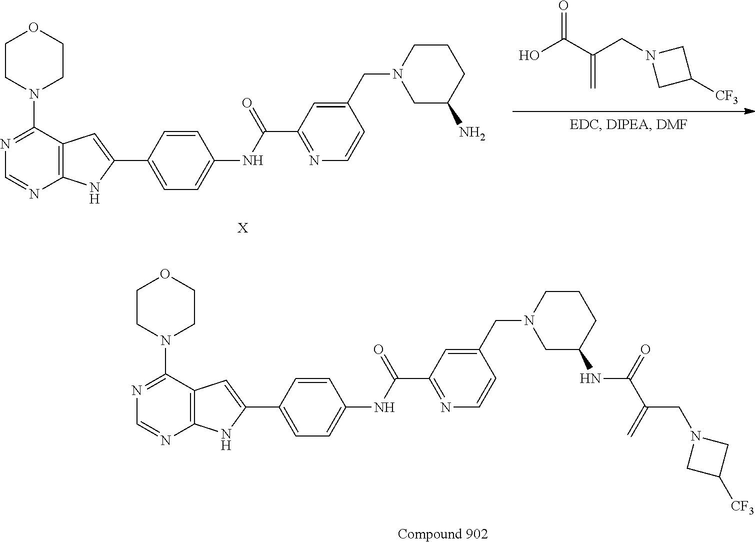

(R)-4-((3-aminopiperidin-1-yl)methyl)-N-(4-(4-morpholino-7H-pyrrolo[2,3-d]pyrimidin-6-yl)phenyl)picolinamide trihydrochloride (50 mg, 0.0804 mmol) was added to a solution of 1-(3-dimethylaminopropyl)-3-ethylcarbodiimide hydrochloride (23 mg, 0.121 mmol), 2-((3-(trifluoromethyl)azetidin-1-yl)methyl)acrylic acid (25 mg, 0.121 mmol) (as a solution in acetonitrile), and N,N-diisopropylethylamine (0.11 mL, 0.643 mmol) in N,N-dimethylformamide (2 mL). After 16 hours at 32° C. the reaction was subjected to preparative HPLC(5-85% acetonitrile/water, 0.1% trifluoroacetic acid). The fractions containing product were combined and subjected to lyophilization, providing Compound 902 (68 mg, 0.0788 mmol, 97% yield).

¹H NMR (400 MHz, MeOD) & 8.74 (d, J=4.9 Hz, 1H), 8.30 (d, J=1.6 Hz, 1H), 8.21 (s, 1H), 7.87 (d, J=8.9 Hz, 2H), 7.77 (d, J=8.8 Hz, 2H), 7.67 (dd, J=4.9, 1.7 Hz, 1H), 7.18 (s, 1H), 6.16 (s, 1H), 5.96 (m, 1H), 4.42 (s, 2H), 4.38-4.26 (m, 2H), 4.21 (m, 2H), 4.11 (m, 1H), 4.06-3.92 (m, 6H), 3.82 (m, 4H), 3.69 (dq, J=16.2, 8.2 Hz, 1H), 3.54-3.39 (m, 1H), 3.34 (m, 1H), 2.96 (m, 2H), 1.96 (m, 2H), 1.87-1.73 (m, 1H), 1.60 (d, J=12.3 Hz, 1H).

LCMS=[M+H]⁺: 704.4; Purity=95%.

### Example 55

### Compound 903

### Preparation of N-(4-(4-(3-oxa-8-azabicyclo[3.2.1]octan-8-yl)-7H-pyrrolo[2,3-d]pyrimidin-6-yl)phenyl)-4-((3-acrylamidoazetidin-1-yl)methyl)picolinamide (Compound 903)

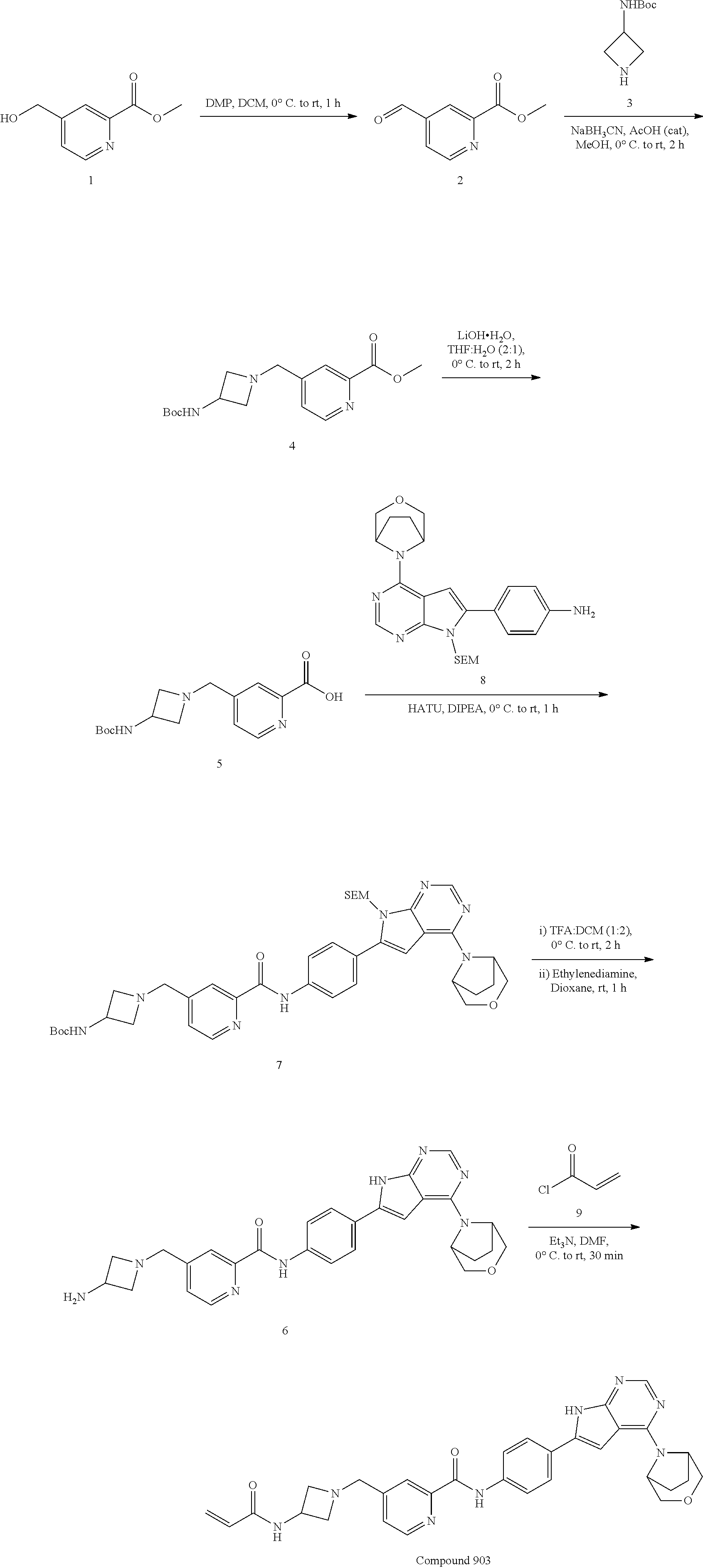

### Synthesis of methyl 4-formylpicolinate (2)

To a stirred solution of methyl 4-(hydroxymethyl)picolinate 1 (1 g, 5.98 mmol) in DCM (30 mL) was added DMP (5.07 g, 12 mmol) at 0° C. The reaction mixture stirred at RT for 1 h. After completion reaction (monitoring by TLC), the reaction mixture was quenched with 1:1 mixture of sat. NaHCO₃ (20 mL) and sat. Na2S2O3 (20 mL) and stirred for 20 min. The reaction mixture was extracted with DCM (3×100 mL). The combined organic layer was washed with brine (2×50 mL), dried over Na₂SO₄, filtered and concentrated under reduced pressure to get the title compound as yellow liquid 2 (900 mg; crude) which was used in next step directly without further purification.

### Synthesis of methyl 4-((3-((tert-butoxycarbonyl)amino) azetidin-1-yl)methyl)picolinate (4)

To a stirred solution of methyl 4-formylpicolinate 2 (800 mg, 4.84 mmol) in MeOH (10 mL) were added and tert-butyl azetidin-3-ylcarbamate 3 (1 g, 5.81 mmol) and AcOH (cat) at 0° C. and stirred at RT for 30 min. Then, NaBH₃CN(609 mg, 9.69 mmol) was added to the reaction mixture at 0° C. and stirred at RT for 2 h. After completion of reaction (LCMS monitoring), the reaction mixture was quenched with sat. NaHCO₃ (10 mL) and extracted with DCM (3×50 mL). The combined organic layer was dried over Na₂SO₄, filtered and concentrated under reduced pressure. The crude was purified by flash chromatography (silica gel 100-200 M, 12 g SNAP) using 2-5% MeOH in DCM to get the title compound as a brown solid 4 (1.2 g, 77%). LCMS=[M+H]⁺: 322.31; Purity=80%.

### Synthesis of 4-((3-((tert-butoxycarbonyl)amino) azetidin-1-yl)methyl)picolinic acid (5)

To a stirred solution of methyl 4-((3-((tert-butoxycarbonyl)amino) azetidin-1-yl)methyl)picolinate 4 (1.1 g, 3.42 mmol) in THF (20 mL) was added LiOH·H₂O (573 mg, 13.6 mmol) dissolved with H₂O (10 mL) dropwise to the reaction mixture at 0° C. The reaction mixture was stirred at RT for 2 h. After completion of reaction (TLC monitoring), the reaction mixture was concentrated under reduced pressure. The crude was dissolved in 25% IPA in CHCl₃ (50 mL) and filtered through sintered funnel. The filtrate was concentrated under reduced pressure to get the desired compound as a pale white solid 5 (860 mg; crude as Li⁺ salt) which was used in the next step directly without any purification.

LCMS=[M+H]⁺: 308.40; Purity=94%.

### Synthesis of tert-butyl(1-((2-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)carbamoyl)pyridin-4-yl)methyl)azetidin-3-yl)carbamate (7)

To an ice cold stirred solution of 4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl) aniline 6 (550 mg, 1.22 mmol) in DMF (10 mL) were added 4-((3-((tert-butoxycarbonyl)amino) azetidin-1-yl)methyl)picolinic acid 5 (458 mg, 1.46 mmol), DIPEA (0.64 mL, 3.65 mmol) and HATU (926 mg, 2.44 mmol) respectively under N₂ atmosphere. The reaction mixture was stirred at RT for 1 h. After completion of reaction (TLC monitoring), the reaction mixture was poured into ice cold water (50 mL) and extracted with EtOAc (3×50 mL). The combined organic layer was dried over Na₂SO₄, filtered and concentrated under reduced pressure. The crude was purified by flash chromatography (silica gel 100-200 M, 12 g SNAP) using 3-5% MeOH in DCM to get the title compound as a white solid 7 (400 mg, 42%)

LCMS=[M+H]⁺: 741.56; Purity=90%.

### Synthesis of N-(4-(4-(3-oxa-8-azabicyclo[3.2.1]octan-8-yl)-7H-pyrrolo[2,3-d]pyrimidin-6-yl)phenyl)-4-((3-aminoazetidin-1-yl)methyl)picolinamide (8)

To a stirred solution of tert-butyl(1-((2-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)carbamoyl)pyridin-4-yl)methyl)azetidin-3-yl)carbamate 7 (400 mg, 0.540 mmol) in DCM (5 mL) was added TFA (2.5 mL) dropwise at 0° C. and stirred at RT for 2 h. After completion of reaction (TLC monitoring), the reaction mixture was concentrated under reduced pressure. The crude was dissolved in 1,4-dioxane (4 mL), followed by 1,2-ethanediamine (36 μL, 0.540 mmol) was added to the reaction mixture at RT and stirred for 1 h. After completion of reaction (LCMS monitoring), the reaction mixture was diluted with 25% IPA in CHCl₃ (50 mL) and washed with water (2×20 mL). The organic layer was dried over Na₂SO₄, filtered and concentrated under reduced pressure to get the desired compound as a white solid 8 (260 mg; crude) which was used in next step directly without further purification.

LCMS=[M+H]′: 511.46; Purity=84%.

### Synthesis of N-(4-(4-(3-oxa-8-azabicyclo[3.2.1]octan-8-yl)-7H-pyrrolo[2,3-d]pyrimidin-6-yl)phenyl)-4-((3-acrylamidoazetidin-1-yl)methyl)picolinamide (Compound 903)

To a stirred solution of N-(4-(4-(3-oxa-8-azabicyclo[3.2.1]octan-8-yl)-7H-pyrrolo[2,3-d]pyrimidin-6-yl)phenyl)-4-((3-aminoazetidin-1-yl)methyl)picolinamide 8 (230 mg, 0.450 mmol) in DMF (5 mL) was added Et₃N(0.19 mL, 1.35 mmol) at 0° C. and stirred for 15 min under N₂ atmosphere. Then, acryl chloride 9 (48.9 mg, 0.541 mmol) dissolved in DMF (1 mL) was added dropwise to the reaction at 0° C. and stirred at RT for 30 min. After completion of reaction (TLC and LCMS monitoring), the reaction mixture was poured into ice cold water (50 mL) and extracted with EtOAc (3×50 mL). The combined organic layer was washed with ice cold brine (2×50 mL), dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get the title compound Compound 903 (33 mg, 13%).

¹H NMR (400 MHz, DMSO-d₆): δ 12.18 (s, 1H), 10.73 (s, 1H), 8.67 (d, J=5.2 Hz, 1H), 8.61 (d, J=7.2 Hz, 1H), 8.17 (s, 1H), 8.08 (s, 1H), 7.99 (d, J=8.8 Hz, 2H), 7.91 (d, J=8.8 Hz, 2H), 7.57 (d, J=3.6 Hz, 1H), 7.12 (d, J=1.6 Hz, 1H), 6.24-6.18 (m, 1H), 6.08 (dd, J=16.8 Hz, 2 Hz, 1H), 5.60 (dd, J=10 Hz, 2.4 Hz, 1H), 4.92 (s, 2H), 4.43-4.38 (m, 1H), 3.37 (s, 2H), 3.67-3.55 (m, 6H), 2.99 (t, J=7.6 Hz, 2H), 2.04-1.96 (m, 4H). LCMS=[M+H]⁺: 565.30; Purity=98%.

### Example 56

### Compound 904 and Compound 905

### Preparation of N-((3R)-1-((2-((1R)-1-((4-(4-(3-oxa-8-azabicyclo[3.2.1]octan-8-yl)-7H-pyrrolo[2,3-d]pyrimidin-6-yl)phenyl)amino)ethyl)pyridin-4-yl)methyl)piperidin-3-yl)-2-(azetidin-1-ylmethyl)acrylamide (Compound 904) and N-((3R)-1-((2-((1S)-1-((4-(4-(3-oxa-8-azabicyclo[3.2.1]octan-8-yl)-7H-pyrrolo[2,3-d]pyrimidin-6-yl)phenyl)amino)ethyl)pyridin-4-yl)methyl)piperidin-3-yl)-2-(azetidin-1-ylmethyl)acrylamide (Compound 905)

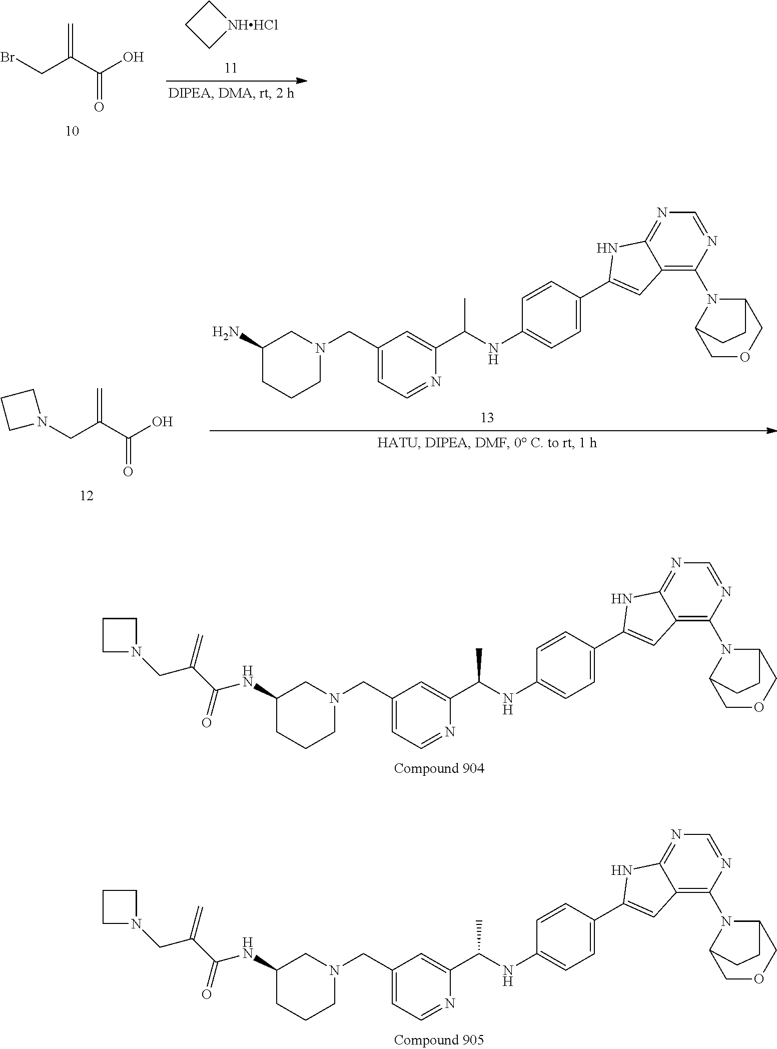

### Synthesis of 2-(azetidin-1-ylmethyl)acrylic acid (12)

To a stirred solution of 2-(azetidin-1-ylmethyl)acrylic acid 10 (1 g, 6.06 mmol) in DMA (10 mL) were added DIPEA (3.17 mL, 18.2 mmol) and azetidine hydrochloride 11 (567 mg, 6.06 mmol) at RT and stirred for 2 h. After completion of reaction (monitoring by TLC and LCMS), the reaction mixture was diluted with ice cold water (50 mL) and extracted with EtOAc (3×50 mL). Aqueous layer was concentrated under reduce pressure. The crude was purified by RP-HPLC to get the title compound as oily liquid 12 (250 mg, 29%). ¹H NMR (400 MHz, DMSO-d₆): >6.39 (s, 1H), 6.05 (s, 1H), 4.03 (t, J=8.4 Hz, 4H), 3.98 (s, 2H), 2.33-2.29 (m, 2H). [CO₂H proton is not appear]

### Synthesis of N-((3R)-1-((2-((1R)-1-((4-(4-(3-oxa-8-azabicyclo[3.2.1]octan-8-yl)-7H-pyrrolo[2,3-d]pyrimidin-6-yl)phenyl)amino)ethyl)pyridin-4-yl)methyl)piperidin-3-yl)-2-(azetidin-1-ylmethyl)acrylamide (Compound 904) and N-((3R)-1-((2-((1S)-1-((4-(4-(3-oxa-8-azabicyclo[3.2.1]octan-8-yl)-7H-pyrrolo[2,3 -d]pyrimidin-6-yl)phenyl)amino)ethyl)pyridin-4-yl)methyl)piperidin-3-yl)-2-(azetidin-1-ylmethyl)acrylamide (Compound 905)

To a stirred solution of 2-(azetidin-1-ylmethyl)acrylic acid 12 (164 mg, 1.16 mmol) and (3R)-1-((2-(1-((4-(4-(3-oxa-8-azabicyclo[3.2.1]octan-8-yl)-7H-pyrrolo[2,3-d]pyrimidin-6-yl)phenyl)amino)ethyl)pyridin-4-yl)methyl)piperidin-3-amine 13 (250 mg 0.464 mmol) in DMF (6 mL) were added DIPEA (0.24 mL, 1.39 mmol) and HATU (353 mg, 0.928 mmol) sequentially at 0° C. under N₂ atmosphere and stirred at RT for 1 h. After completion of reaction (monitoring by TLC and LCMS), the reaction mass was poured into ice cold water (30 mL) and extracted with 25% IPA in CHCl₃ (3×50 mL). The combined organic layer was dried over Na₂SO₄, filtered and concentrated under reduced pressure. The crude was purified by RP-HPLC [15-85% 5 mM Ammonium Bicarbonate in Water/ACN; Column: Symmetry C18 (19*300) 7μ] to get the desired products as separable diastereomers Compound 904 (25 mg, 8%) and Compound 905 (15 mg, 5%).

Analytical data for Compound 904: 1H NMR (400 MHz, DMSO-d₆): δ 11.85 (s, 1H), 8.69 (brs, 1H), 8.44 (d, J=5.2 Hz, 1H), 8.10 (s, 1H), 7.54 (d, J=8.4 Hz, 2H), 7.32 (s, 1H), 7.18 (d, J=5.2 Hz, 1H), 6.75 (s, 1H), 6.52 (d, J=8.8 Hz, 2H), 6.42 (d, J=7.2 Hz, 1H), 5.75 (brs, 1H), 5.36 (brs, 1H), 4.84 (brs, 2H), 4.57-4.53 (m, 1H), 3.82-3.81 (m, 1H), 3.66 (d, J=10.8 Hz, 2H), 3.58 (d, J=10.8 Hz, 2H), 3.52-3.32 (m, 2H), 3.18 (brs, 2H), 3.09 (t, J=6.8 Hz, 4H), 2.53-2.49 (m, 1H), 2.28-2.22 (m, 1H), 2.10-2.02 (m, 2H), 2.00-1.92 (m, 6H), 1.57-1.55 (m, 2H), 1.45 (d, J=6.8 Hz, 3H), 1.38-1.33 (m, 1H), 1.27-1.23 (m, 1H).

LCMS=[M+H]⁺: 662.39; Purity=98.80%.

Analytical data for Compound 905: 1H NMR (400 MHz, DMSO-d₆): δ 11.86 (s, 1H), 8.77 (brs, 1H), 8.45 (d, J=5.2 Hz, 1H), 8.10 (s, 1H), 7.54 (d, J=8.4 Hz, 2H), 7.31 (s, 1H), 7.21 (d, J=4 Hz, 1H), 6.75 (s, 1H), 6.54 (d, J=8.8 Hz, 2H), 6.38 (d, J=6.8 Hz, 1H), 5.77 (d, J=2 Hz, 1H), 5.37 (brs, 1H), 4.84 (brs, 2H), 4.58-4.51 (m, 1H), 3.85-3.83 (m, 1H), 3.67 (d, J=9.6 Hz, 2H), 3.58 (d, J=10.8 Hz, 2H), 3.51-3.43 (m, 2H), 3.17 (d, J=4.8 Hz, 2H), 3.119 (t, J=6.8 Hz, 4H), 2.29-2.23 (m, 1H), 2.20-2.16 (m, 2H), 2.01-1.92 (m, 6H), 1.59-1.57 (m, 2H), 1.45 (d, J=6.4 Hz, 4H), 1.33-1.31 (m, 1H). [1 proton is may merged in solvent peak]LCMS=[M+H]⁺: 662.35; Purity=97.56%.

### Example 57

### Compound 906

### Preparation of 1-(4-(5-((4-(4-morpholino-7H-pyrrolo[2,3-d]pyrimidin-6-yl)phenyl)amino)pyrimidin-2-yl)piperazin-1-yl)prop-2-en-1-one (Compound 906)

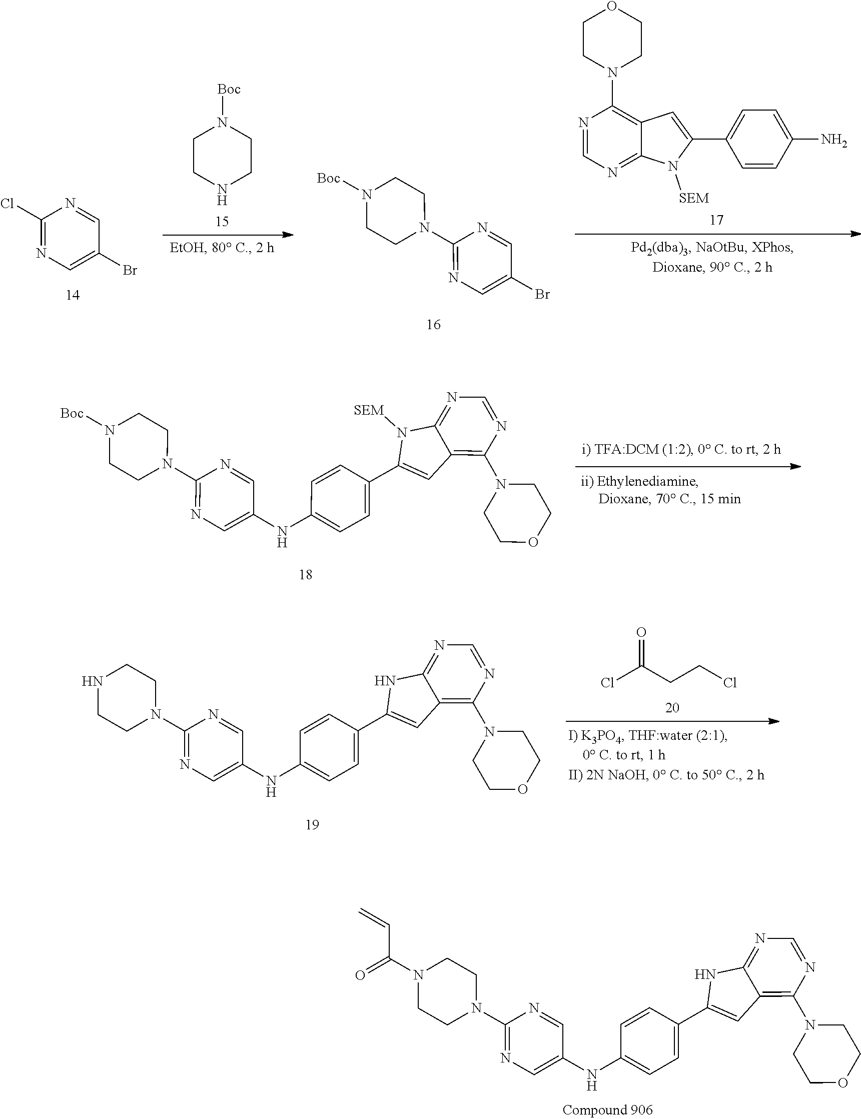

### Synthesis of tert-butyl 4-(5-bromopyrimidin-2-yl)piperazine-1-carboxylate (16)

To a stirred solution of 5-bromo-2-chloropyrimidine 14 (1 g, 5.17 mmol) in EtOH (10 mL) was added tort-butyl piperazine-1-carboxylate 15 (963 mg, 5.17 mmol) at RT and stirred at 80° C. for 2 h. After completion of reaction (monitoring by TLC), the reaction mixture was concentrated under reduced pressure. The crude was purified by flash chromatography using 10-15% EtOAc in heptane to get the desired compound as a brown semi-solid 16 (1 g, 56%).

LCMS=[M+H]⁺: 343.07; Purity=70%.

### Synthesis of tert-butyl 4-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperazine-1-carboxylate (18)

To a stirred solution of tert-butyl 1-(5-bromo-2-pyrimidinyl)-4-piperidinecarboxylate 16 (800 mg, 2.34 mmol) and 4-(4-morpholino-7-((2-(trimethylsilyl)ethoxy)methyl)-7H-pyrrolo[2,3-d]pyrimidin-6-yl) aniline 17 (995 mg, 2.34 mmol) in 1,4-dioxane (15 mL) was added NaO′Bu (337 mg, 3.51 mmol) at RT. The reaction mixture was degassed with N₂ gas for 10 min, followed by XPhos (111 mg, 0.234 mmol) and Pd₂(dba)₃ (189 mg, 0.234 mmol) were added to the reaction mixture and stirred at 90° C. for 2 h. After completion of reaction (TLC and LCMS monitoring), the reaction mixture was diluted with water (20 mL) and extracted with EtOAc (3×50 mL). The combined organic layer was dried over Na₂SO₄, filtered and evaporated under reduced pressure. The crude was purified by flash chromatography (silica gel 100-200 M, 12 g SNAP) using 20-30% EtOAc in heptane to get the title compound as a light yellow solid 18 (1.2 g, 61%) LCMS=[M+H]⁺: 688.37; Purity=82%.

### Synthesis of N-(4-(4-morpholino-7H-pyrrolo[2,3-d]pyrimidin-6-yl)phenyl)-2-(piperazin-1-yl)pyrimidin-5-amine (19)

To a stirred solution of tert-butyl 4-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperazine-1-carboxylate 18 (1.2 g, 1.74 mmol) in DCM (20 mL) was added TFA (10 mL) dropwise at 0° C. and stirred at RT for 2 h. After consumed of starting material (monitoring by TLC), the reaction mixture was concentrated under reduced pressure. The crude was dissolved in 1,4-dioxane (10 mL) and 1,2-ethanediamine (0.35 mL, 5.23 mmol) was added to the reaction mixture at RT and stirred at 70° C. for 15 min. After completion of reaction (LCMS monitoring), the reaction mixture was diluted with 25% IPA in CHCl₃ (100 mL) and washed with ice cold water (2×50 mL). The organic layer was dried over Na₂SO₄, filtered and concentrated under reduced pressure to get the desired compound as a yellow solid (900 mg; crude) which was used in next step directly without further purification. LCMS=[M+H]⁺: 458.23; Purity=77%.

### Synthesis of 1-(4-(5-((4-(4-morpholino-7H-pyrrolo[2,3-d]pyrimidin-6-yl)phenyl)amino)pyrimidin-2-yl)piperazin-1-yl)prop-2-en-1-one (Compound 906)

To a stirred solution of N-(4-(4-morpholino-7H-pyrrolo[2,3-d]pyrimidin-6-yl)phenyl)-2-(piperazin-1-yl)pyrimidin-5-amine 19 (500 mg, 1.09 mmol) in THF (10 mL) was added K₃PO₄ (464 mg, 2.19 mmol) dissolved in water (5 mL) at 0° C. Then, 3-chloropropionyl chloride 20 (0.13 mL, 1.31 mmol) dissolved in THF (2 mL) was added dropwise to the reaction mixture at 0° C. and was stirred at RT for 1 h. After consumed of starting material (monitoring by TLC), 2N NaOH (6.5 mL, 13.08 mmol) was added to the reaction mixture at 0° C. and stirred at 50° C. for 2 h. After completion of reaction (TLC and LCMS monitoring), the reaction mixture was diluted with water (50 mL) and extracted with 2-MeTHF (3×50 mL). The combined organic layer was dried over Na₂SO₄, filtered and concentrated under reduced pressure. The crude was purified by RP-HPLC purification[5-95% 5 mM Ammonium Bicarbonate in Water/ACN; Column: X Select CSH C18 (19*250) 10μ] to get the title compound Compound 906 (147 mg, 26%).

¹H NMR (400 MHz, DMSO-d₆): δ 12.03 (s, 1H), 8.34 (s, 2H), 8.14 (s, 1H), 7.95 (s, 1H), 7.71 (d, J=8.8 Hz, 2H), 6.92 (d, J=2 Hz, 1H), 6.89-6.82 (m, 3H), 6.15 (dd, J=16.8 Hz, 2.4 Hz, 1H), 5.72 (dd, J=10.4 Hz, 2.4 Hz, 1H), 3.86-3.84 (m, 4H), 3.74-3.72 (m, 8H), 3.69-3.61 (m, 4H).

LCMS=[M+H]⁺: 512.30; Purity=99.59%.

### Example 58

### Compound 907

### Preparation of (E)-1-(3,3-difluoroazetidin-1-yl)-4-(4-(5-((4-(4-morpholino-7H-pyrrolo[2,3-d]pyrimidin-6-yl)phenyl)amino)pyrimidin-2-yl)piperazin-1-yl)but-2-en-1-one (Compound 907)

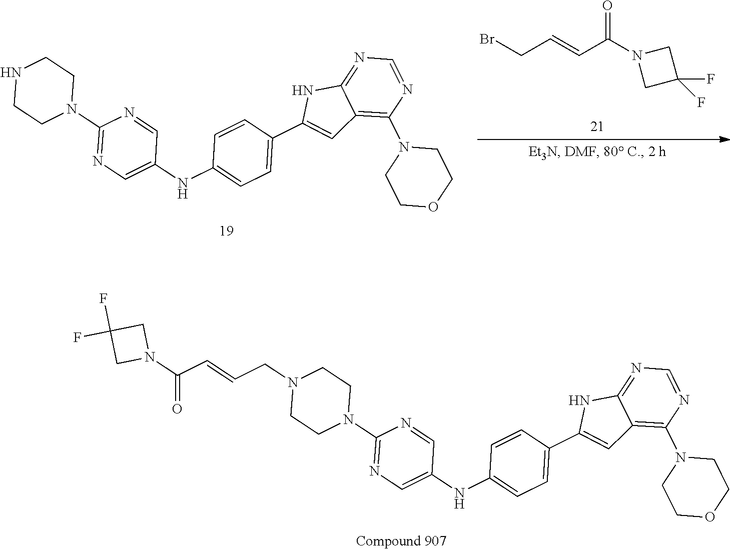 [SMILES: c1nc(N2CCOCC2)c2cc(-c3ccc(Nc4cnc(N5CCNCC5)nc4)cc3)[nH]c2n1 ; O=C(/C=C/CBr)N1CC(F)(F)C1 ; O=C(/C=C/CN1CCN(c2ncc(Nc3ccc(-c4cc5c(N6CCOCC6)ncnc5[nH]4)cc3)cn2)CC1)N1CC(F)(F)C1]

To an ice cold stirred solution of N-(4-(4-morpholino-7H-pyrrolo[2,3-d]pyrimidin-6-yl)phenyl)-2-(piperazin-1-yl)pyrimidin-5-amine 19 (200 mg, 0.437 mmol) in DMF (5 mL) were added (E)-4-bromo-1-(3,3 -difluoroazetidin-1-yl)but-2-en-1-one 21 (157 mg, 0.656 mmol) and Et₃N(0.19 mL, 1.31 mmol) at RT and stirred at 80° C. for 2 h. After completion of reaction (LCMS monitoring), the reaction mixture was diluted with ice cold water (20 mL) and extracted with 25% IPA in CHCl₃ (3×30 mL). The combined organic layer was dried over Na₂SO₄, filtered and evaporated under reduced pressure. The crude was purified by RP-HPLC [15-85% 5 mM Ammonium Bicarbonate in Water/ACN; Column: X Select CSH C18 (19*250) 10μ] to get the title compound Compound 907 (45 mg, 17%).

¹H NMR (400 MHz, DMSO-d₆): δ 12.03 (s, 1H), 8.31 (s, 2H), 8.14 (s, 1H), 7.92 (s, 1H), 7.70 (d, J=8.4 Hz, 2H), 6.92 (d, J=1.6 Hz, 1H), 6.82 (d, J=8.4 Hz, 2H), 6.72-6.65 (m, 1H), 6.20-6.16 (m, 1H), 4.69 (t, J=10.8 Hz, 2H), 4.34 (t, J=11.6 Hz, 2H), 3.86-3.84 (m, 4H), 3.75-3.70 (m, 8H), 3.16 (brs, 2H), 2.49-2.46 (m, 4H). LCMS=[M+H]⁺: 617.25; Purity=97.73%.

### Example 59

### Compound 908

### Preparation of 1-(4-hydroxy-4-(((4-(4-morpholino-7H-pyrrolo[2,3-d]pyrimidin-6-yl)phenyl)amino)methyl)-[1,4′-bipiperidin]-1′-yl)prop-2-en-1-one (Compound 908)

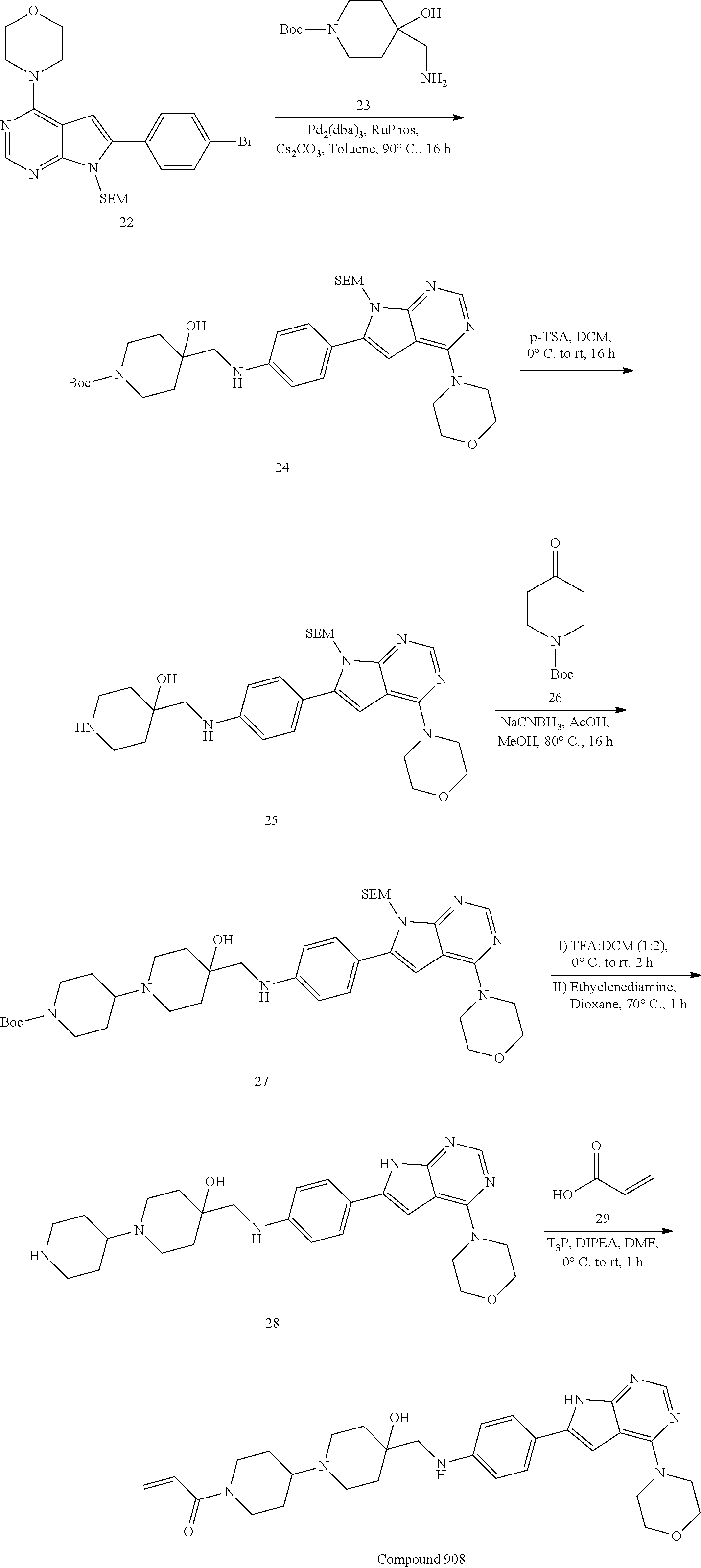

### Synthesis of tert-butyl 4-hydroxy-4-(((4-(4-morpholino-7-((2-(trimethylsilyl)ethoxy)methyl)-7H-pyrrolo[2,3-d]pyrimidin-6-yl)phenyl)amino)methyl)piperidine-1-carboxylate (24)

To a stirred solution of 4-(6-(4-bromophenyl)-7-((2-(trimethylsilyl)ethoxy)methyl)-7H-pyrrolo[2,3-d]pyrimidin-4-yl) morpholine 22 (2.5 g, 5.11 mmol) and tert-butyl 4-(aminomethyl)-4-hydroxypiperidine-1-carboxylate 23 (1.76 g, 7.66 mmol) in toluene (30 mL) was added Cs₂CO₃ (5 g, 15.3 mmol) at RT. The reaction mixture was degassed with N₂ gas for 10 min, followed by RuPhos (477 mg, 1.02 mmol) and Pd₂(dba)₃ (468 mg, 0.51 1 mmol) were added to the reaction mixture and stirred at 90° C. for 16 h. After the completion of reaction (TLC and LCMS monitoring), the reaction mixture was diluted with water (50 mL) and extracted with EtOAc (3×50 mL). The combined organic layer was dried over Na₂SO₄, filtered and evaporated under reduced pressure. The crude was purified by flash chromatography (silica gel 100-200 M, 40 g SNAP) using 50-60% EtOAc in heptane to get the desired compound as a yellow solid 24 (2.8 g, 47%).

LCMS=[M+H]⁺: 639.40; Purity=55%.

### Synthesis of 4-(((4-(4-morpholino-7-((2-(trimethylsilyl)ethoxy)methyl)-7H-pyrrolo[2,3-d]pyrimidin-6-yl)phenyl)amino)methyl)piperidin-4-ol (25)

To a stirred solution of tert-butyl 4-hydroxy-4-(((4-(4-morpholino-7-((2-(trimethylsilyl)ethoxy)methyl)-7H-pyrrolo[2,3-d]pyrimidin-6-yl)phenyl)amino)methyl)piperidine-1-carboxylate 24 (2.8 g, 4.38 mmol) in DCM (30 mL) was added p-TSA (3.33 g, 17.5 mmol) at 0° C. and stirred at RT for 16 h. After completion of reaction (TLC and LCMS monitoring), the reaction mixture was basified with NaHCO₃ and extracted with DCM (3×100 mL). The combined organic layer was dried over Na₂SO₄, filtered and evaporated under reduced pressure to get the title compound as a brown solid 25 (2.3 g; crude) which was used in next step directly without any purification.

LCMS=[M+H]⁺: 539.29

### Synthesis of tert-butyl 4-hydroxy-4-(((4-(4-morpholino-7-((2-(trimethylsilyl)ethoxy)methyl)-7H-pyrrolo[2,3-d]pyrimidin-6-yl)phenyl)amino)methyl)-[1,4′-bipiperidine]-1′-carboxylate (27)

To a stirred solution of 4-(((4-(4-morpholino-7-((2-(trimethylsilyl)ethoxy)methyl)-7H-pyrrolo[2,3-d]pyrimidin-6-yl)phenyl)amino)methyl)piperidin-4-ol 25 (1.25 g, 2.32 mmol) in MeOH (20 mL) were added tert-butyl 4-oxopiperidine-1-carboxylate 26 (1.43 g, 4.64 mmol) and AcOH (0.26 mL, 4.64 mmol) at 0° C. under N2 atmosphere and stirred for 30 min. Then, NaBH₃CN(555 mg, 9.28 mmol) was added to the reaction mixture at 0° C. and stirred at 80° C. for 16 h. After completion of reaction (TLC and LCMS monitoring), the reaction mixture was quenched with sat. NaHCO₃ and extracted with 25% IPA in CHCl₃ (3×50 mL). The combined organic layer was dried over Na₂SO₄, filtered and concentrated under reduced pressure. The crude was purified by flash chromatography on (silica gel 100-200 M, 24 g SNAP) using 7-10% MeOH in DCM to get the title compound as a sticky yellow 27 (1 g, 42%).

LCMS=[M+H]⁺: 722.53; Purity=71%.

### Synthesis of 4-(((4-(4-morpholino-7H-pyrrolo[2,3-d]pyrimidin-6-yl)phenyl)amino)methyl)-[1,4′-bipiperidin]-4-ol (28)

To a stirred solution of tert-butyl 4-hydroxy-4-(((4-(4-morpholino-7-((2-(trimethylsilyl)ethoxy)methyl)-7H-pyrrolo[2,3-d]pyrimidin-6-yl)phenyl)amino)methyl)-[1,4′-bipiperidine]-1′-carboxylate 27 (1 g, 1.39 mmol) in DCM (10 mL) was added TFA (5 mL) dropwise at 0° C. and stirred at RT for 2 h. After consumed of starting material (monitoring by TLC), the reaction mixture was concentrated under reduced pressure. The crude was dissolved in 1,4-dioxane (10 mL) and 1,2-ethanediamine (0.14 mL, 2.08 mmol) was added to the reaction mixture at RT and stirred at 70° C. for 1 h. After completion of reaction (LCMS monitoring), the reaction mixture was diluted with water (50 mL) and extracted with 25% IPA in CHCl₃ (3×50 mL). The combined organic layer was dried over Na₂SO₄, filtered and concentrated under reduced pressure to get the desired compound as a yellow solid 28 (600 mg; crude) which was used in the next step directly without any purification.

LCMS=[M+H]⁺: 492.48; Purity=56%.

### Synthesis of 1-(4-hydroxy-4-(((4-(4-morpholino-7H-pyrrolo[2,3-d]pyrimidin-6-yl)phenyl)amino)methyl)-[1,4′-bipiperidin]-1′-yl)prop-2-en-1-one (Compound 908)

To a solution of 4-(((4-(4-morpholino-7H-pyrrolo[2,3-d]pyrimidin-6-stirred yl)phenyl)amino)methyl)-[1,4′-bipiperidin]-4-ol 28 (500 mg, 1.02 mmol) in DMF (5 mL) were added acrylic acid 29 (58.6 mg, 0.814 mmol), DIPEA (0.5 mL, 3.05 mmol) and T3P (1.2 mL, 203 mmol; 50% w/v in EtOAc) sequentially at 0° C. and stirred at RT for 1 h. After completion of reaction (TLC monitoring), the reaction mixture was poured into ice cold water (20 mL) and extracted with 25% IPA in CHCl₃ (3×20 mL). The combined organic layer was dried over Na₂SO₄, filtered and evaporated under reduced pressure. The crude was purified by RP-HPLC [5-95% 5 mM ammonium bicarbonate in water/ACN; Column: X select phenyl hexyl (19*250) 5μ] to get the title Compound 908 (21 mg, 4%).

¹H NMR (400 MHz, DMSO-d₆): δ 11.91 (s, 1H), 8.12 (s, 1H), 7.59 (d, J=8.8 Hz, 2H), 6.83-6.75 (m, 2H), 6.67 (d, J=8.8 Hz, 2H), 6.06 (dd, J=16.8 Hz, 2.4 Hz, 1H), 5.63 (dd, J=10.4 Hz, 2.8 Hz, 1H), 5.58 (t, J=9.8 Hz, 1H), 4.44-4.41 (m, 1H), 4.26 (s, 1H), 4.06-4.04 (m, 1H), 3.83-3.82 (m, 4H), 3.74-3.72 (m, 4H), 3.03-2.97 (m, 3H), 2.58-2.55 (m, 2H), 2.47-2.44 (m, 2H), 1.77-1.74 (m, 2H), 1.55-1.52 (m, 4H), 1.30-1.26 (m, 2H). [2 protons are may merged in solvent peak]

LCMS=[M+H]⁺: 546.33; Purity=97.40%.

### Example 60

### Compound 909

### Preparation of 1-(3-(4-(2,2,2-trifluoro-1-((4-(4-morpholino-7H-pyrrolo[2,3-d]pyrimidin-6-yl)phenyl)amino)ethyl)piperidin-1-yl) azetidin-1-yl)prop-2-en-1-one (Compound 909)

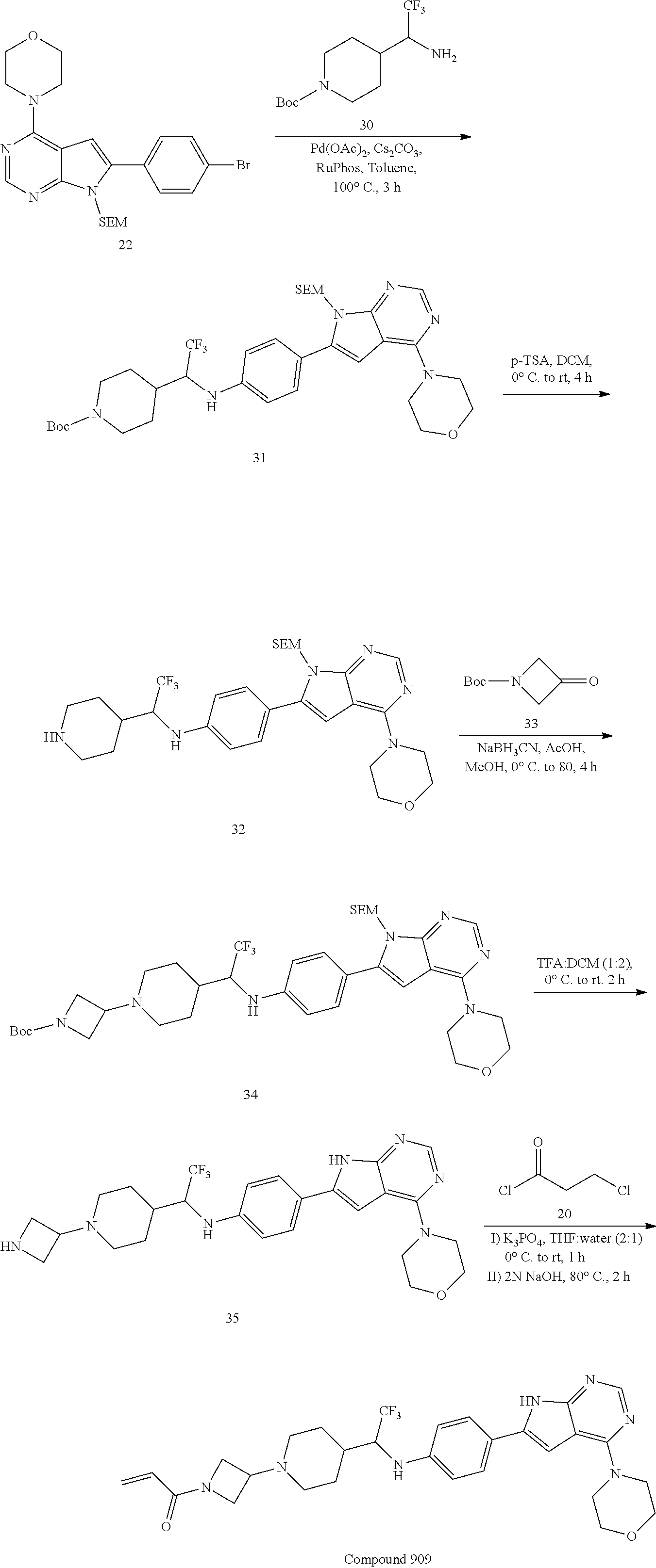

### Synthesis of tert-butyl 4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidine-1-carboxylate (31)

To a stirred solution of 4-(6-(4-bromophenyl)-7-((2-(trimethylsilyl)ethoxy)methyl)-7H-pyrrolo[2,3-d]pyrimidin-4-yl) morpholine 22 (3.5 g, 7.15 mmol) and tert-butyl 4-(1-amino-2,2,2-trifluoroethyl)piperidine-1-carboxylate 30 (2.42 g, 8.58 mmol) in toluene (20 mL) was added Cs₂CO₃ (6.99 g, 21.5 mmol) at RT. The reaction mixture was degassed with N₂ gas for 10 min, followed by RuPhos (667 mg, 1.43 mmol) and Pd(OAc) 2 (161 mg, 0.715 mmol) were added to the reaction mixture and stirred at 100° C. for 3 h. After completion of reaction (monitored by TLC), the reaction mixture was diluted with water (100 mL) and extracted with EtOAc (3×100 mL). The combined organic layer was washed with brine (2×50 mL), dried over Na₂SO₄, filtered and evaporated under reduced pressure. The crude was purified by flash chromatography (silica gel 100-200M, 40 g SNAP) using 65-70% EtOAc in heptane to get the desired product as a pale yellow solid 31 (3 g, 60%). LCMS=[M+H]⁺: 691.65; Purity=65%

### Synthesis of 4-(4-morpholino-7-((2-(trimethylsilyl)ethoxy)methyl)-7H-pyrrolo[2,3-d]pyrimidin-6-yl)-N-(2,2,2-trifluoro-1-(piperidin-4-yl)ethyl) aniline (32)

To a stirred solution of tert-butyl 4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidine-1-carboxylate 31 (2 g, 2.89 mmol) in DCM (20 mL) was added p-TSA (2.2 g, 11.6 mmol) at 0° C. and stirred at RT for 4 h. After completion of reaction (LCMS monitoring), the reaction mixture was basified with sat. NaHCO₃ and extracted with 25% IPA in CHCl₃ (3×50 mL). The combined organic layer was dried over Na₂SO₄, filtered and concentrated under reduced pressure to get the title compound as a brown solid 32 (1.2 g; crude) which was used in next step directly without purification.

LCMS=[M+H]⁺: 591.46; Purity=72%.

### Synthesis of tert-butyl 3-(4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidin-1-yl) azetidine-1-carboxylate (34)

To a stirred solution of 4-(4-morpholino-7-((2-(trimethylsilyl)ethoxy)methyl)-7H-pyrrolo[2,3-d]pyrimidin-6-yl)-N-(2,2,2-trifluoro-1-(piperidin-4-yl)ethyl) aniline 32 (600 mg, 1.02 mmol) and tert-butyl 3-oxoazetidine-1-carboxylate 33 (348 mg, 2.03 mmol) in MeOH (10 mL) was added AcOH (0.12 mL, 2.03 mmol) at 0° C. and stirred at RT for 30 min. Then, NaBH₃CN(128 mg, 2.03 mmol) dissolved with MeOH (1 mL) was added to the reaction mixture at 0° C. and stirred at 80° C. for 4 h. After completion of reaction (TLC and LCMS monitoring), the reaction mixture was quenched with sat. NaHCO₃, extracted with 25% IPA in CHCl₃ (3×50 mL). The combined organic layer was dried over Na₂SO₄, filtered and concentrated under reduced pressure. The crude was purified by flash chromatography (silica gel 100-200 M, 12 g SNAP) using 5-6.5% MeOH in DCM to get the title compound as an off white solid 34 (700 mg, 69%).

LCMS=[M+H]⁺: 746.53; Purity=74%

### Synthesis of N-(1-(1-(azetidin-3-yl)piperidin-4-yl)-2,2,2-trifluoroethyl)-4-(4-morpholino-7H-pyrrolo[2,3-d]pyrimidin-6-yl) aniline (35)

To a stirred solution of tert-butyl 3-(4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidin-1-yl) azetidine-1-carboxylate 34 (700 mg, 0.938 mmol) in DCM (10 mL) was added TFA (5 mL) dropwise at 0° C. and stirred at RT for 2 h. After completion of reaction (TLC and LCMS monitoring), the reaction mixture was concentrated under reduced pressure. The crude was triturated with Et₂O, pentane and dried under reduced pressure to get the title compound as a yellow solid 35 (500 mg; crude as TFA salt) which was used in next step directly without further purification.

LCMS=[M+H]⁺: 516.24; Purity=72%

### Synthesis of 1-(3-(4-(2,2,2-trifluoro-1-((4-(4-morpholino-7H-pyrrolo[2,3-d]pyrimidin-6-yl)phenyl)amino)ethyl)piperidin-1-yl) azetidin-1-yl)prop-2-en-1-one (Compound 909)

To a stirred solution of N-(1-(1-(azetidin-3-yl)piperidin-4-yl)-2,2,2-trifluoroethyl)-4-(4-morpholino-7H-pyrrolo[2,3-d]pyrimidin-6-yl) aniline 35 (400 mg, 0.776 mmol) in THF (6 mL) was added K₃PO₄ (823 mg, 3.88 mmol) dissolved in water (3 mL) at 0° C. Then, 3-chloropropionyl chloride 20 (89 μL, 0.931 mmol) dissolved in THF (2 mL) was added dropwise to the reaction mixture at 0° C. and stirred at RT for 1 h. After consumed of starting material (monitoring by TLC), 2N NaOH (4.5 mL, 9.31 mmol) was added dropwise at RT and stirred at 60° C. for 2 h. After completion of reaction (LCMS monitoring), the reaction mixture was diluted with water (50 mL) and extracted with 2-MeTHF (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get the title Compound 909 (18 mg, 4%).

¹H NMR (400 MHz, DMSO-d₆): δ 11.96 (s, 1H), 8.13 (s, 1H), 7.64 (d, J=8.8 Hz, 2H), 6.87-6.85 (m, 3H), 6.31-6.23 (m, 1H), 6.09-6.05 (m, 2H), 5.64 (d, J=10.4 Hz, 1H), 4.34-4.28 (m, 1H), 4.22-4.17 (m, 1H), 3.97-3.95 (m, 1H), 3.94-3.92 (m, 1H), 3.89-3.83 (m, 4H), 3.79-3.72 (m, 4H), 3.69-3.65 (m, 1H), 3.09-3.03 (m, 1H), 2.87-2.77 (m, 2H), 1.77-1.62 (m, 5H), 1.53-1.42 (m, 2H).

LCMS=[M+H]⁺: 570.37; Purity=98.63%

### Example 61

### Compound 910

### Preparation of N-(1-(5-((4-(4-morpholino-7H-pyrrolo[2,3-d]pyrimidin-6-yl)phenyl)amino)pyrimidin-2-yl)piperidin-4-yl)acrylamide (Compound 910)

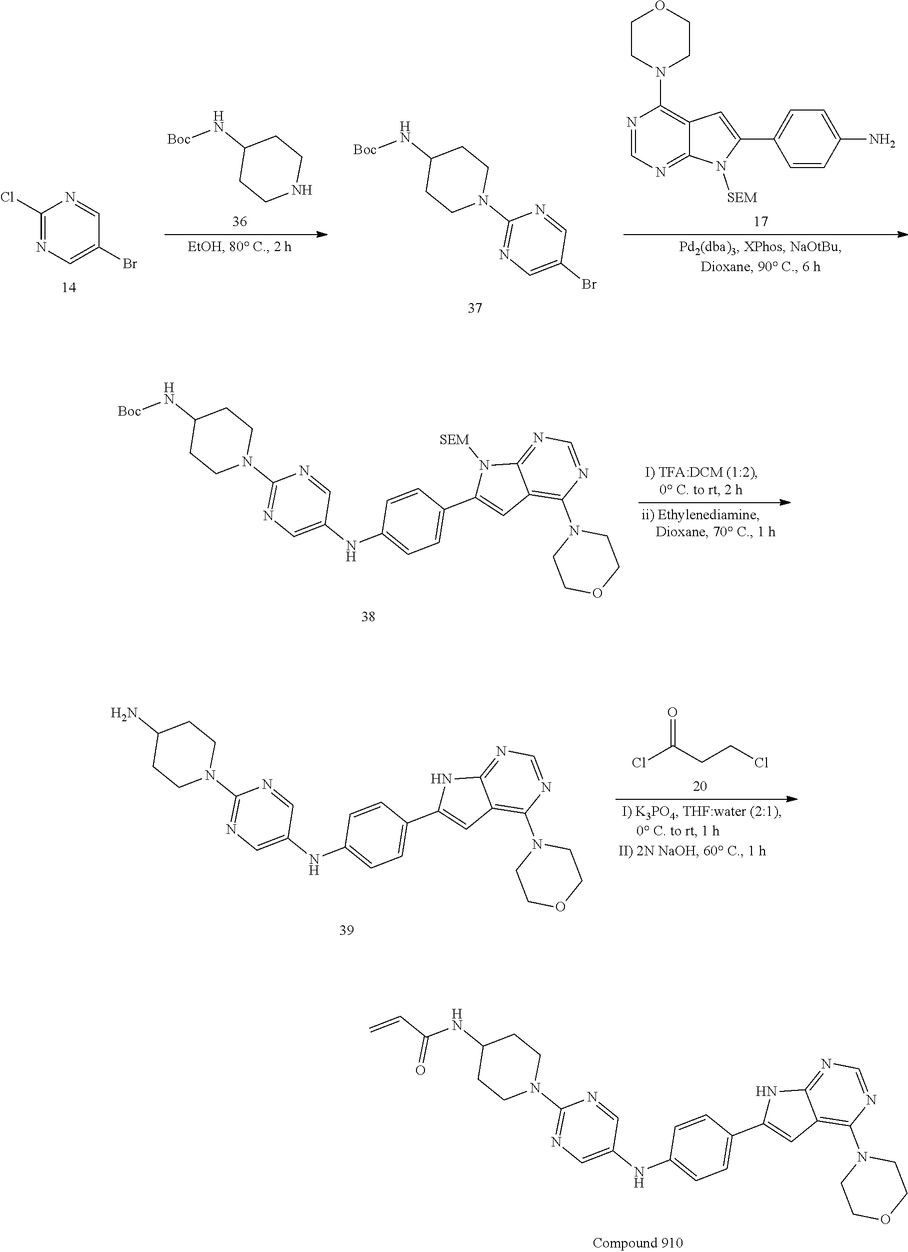

### Synthesis of tert-butyl[1-(5-bromo-2-pyrimidinyl)-4-piperidyl]carbamate (37)

To a stirred solution of 5-bromo-2-chloropyrimidine 14 (1 g, 5.17 mmol) in EtOH (20 mL) was added tert-butyl 4-piperidylcarbamate 36 (2.07 g, 10.3 mmol) at RT and stirred at 80° C. for 2 h. After completion of reaction (TLC monitoring), the reaction mixture was evaporated under reduced pressure. The crude was diluted with water (50 mL) and extracted with EtOAc (3×50 mL). The combined organic layer was dried over Na₂SO₄, filtered and evaporated under reduced pressure to get the title compound as a brown solid 37 (1 g; crude) which was used in next step directly without purification.

LCMS=[M+H]′: 356.08; Purity=70%.

### Synthesis of tert-butyl(1-{5-[p-(4-morpholino-1-{[2-(trimethylsilyl)ethoxy]methyl}-1H-1,5,7-triazainden-2-yl)phenylamino]-2-pyrimidinyl}-4-piperidyl)carbamate (38)

To a stirred solution of tert-butyl[1-(5-bromo-2-pyrimidinyl)-4-piperidyl]carbamate 37 (1 g, 2.8 mmol) in 1,4-dioxane (15 mL) were added p-(4-morpholino-1-{[2-(trimethylsilyl)ethoxy]methyl}-1H-1,5,7-triazainden-2-yl) aniline 17 (1.19 g, 2.8 mmol) and NaO′Bu (404 mg, 4.2 mmol) at RT. The reaction mixture was degassed with N₂ gas for 10 min, followed by XPhos (267 mg, 0.560 mmol) and Pd₂(dba)₃ (227 mg, 0.280 mmol) were added to the reaction mixture and stirred at 90° C. for 6 h. After completion of reaction (TLC monitoring), the reaction mass was diluted with water (50 mL) and extracted with EtOAc (3×50 mL). The combined organic layer was washed with brine solution (2×50 mL), dried over Na₂SO₄, filtered and concentrated under reduced pressure. The crude was purified by flash chromatography using 40-50% EtOAc in heptane to get the title compound as a pale yellow solid 38 (1.2 g, 61%).

LCMS=[M+H]⁺: 702.77; Purity=75%

### Synthesis of 2-(4-amino-1-piperidyl)-5-[p-(4-morpholino-1H-1,5,7-triazainden-2-yl)phenylamino]pyrimidine (39)

To a stirred solution of tert-butyl(1-{5-[p-(4-morpholino-1-{[2-(trimethylsilyl)ethoxy]methyl}-1H-1,5,7-triazainden-2-yl)phenylamino]-2-pyrimidinyl}-4-piperidyl)carbamate 38 (1.2 g, 1.71 mmol) in DCM (20 mL) was added TFA (10 mL) dropwise at 0° C. and stirred at RT for 2 h. After consumed of starting material (monitoring by TLC), the reaction mixture was concentrated under reduced pressure. The crude was dissolved in 1,4-dioxane (10 mL) and 1,2-ethanediamine (0.17 mL, 2.56 mmol) was added to the reaction mixture at RT and stirred at 70° C. for 1 h. After completion of reaction (LCMS monitoring), the reaction mixture was diluted with water (50 mL) and extracted with 25% IPA in CHCl₃ (3×50 mL). The combined organic layer was dried over Na₂SO₄, filtered and concentrated under reduced pressure to get the title compound as a yellow solid 39 (700 mg; crude) which was used in next step directly without further purification.

LCMS=[M+H]⁺: 472.29; Purity=70%

### Synthesis of N-(1-{5-[p-(4-morpholino-1H-1,5,7-triazainden-2-yl)phenylamino]-2-pyrimidinyl}-4-piperidyl)acrylamide (Compound 910)

To a stirred solution of 2-(4-amino-1-piperidyl)-5-[p-(4-morpholino-1H-1,5,7-triazainden-2-yl)phenylamino]pyrimidine 39 (500 mg, 1.06 mmol) in THF (6 mL) was added K₃PO₄ (450 mg, 2.12 mmol) dissolved in water (3 mL) at 0° C. Then, 3-chloropropionyl chloride 20 (0.12 mL, 0.931 mmol) dissolved in THF (2 mL) was added dropwise to the reaction mixture at 0° C. and stirred at RT for 1 h. After consumed of starting material (monitoring by TLC), 2N NaOH (6.36 mL, 12.7 mmol) was added dropwise at RT and stirred at 60° C. for 1 h. After completion of reaction (LCMS monitoring), the reaction mixture was diluted in water (50 mL) and extracted with 2-MeTHF (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get the title compound Compound 910 (140 mg, 25%).

¹H NMR (400 MHz, DMSO-d₆): δ 12.72 (s, 1H), 8.31-8.29 (m, 3H), 8.06 (d, J=7.6 Hz, 1H), 7.98 (brs, 1H), 7.73 (d, J=8.8 Hz, 2H), 7.13 (s, 1H), 6.83 (d, J=8.8 Hz, 2H), 6.24-6.17 (m, 1H), 6.09 (dd, J=16.8 Hz, 2.4 Hz, 1H), 5.58 (dd, J=10 Hz, 2.4 Hz, 1H), 4.50-4.47 (m, 2H), 3.96-3.91 (m, 4H), 3.80-3.78 (m, 4H), 3.08 (t, J=10.8 Hz, 2H), 1.85-1.83 (m, 2H), 1.41-1.31 (m, 2H). [1H may merged in solvent peak]LCMS=[M+H]⁺: 526.29; Purity=99.87%.

### Example 62

### Compound 911

### Preparation of (S)—N-(1-(5-((4-(4-morpholino-7H-pyrrolo[2,3-d]pyrimidin-6-yl)phenyl)amino)pyrimidin-2-yl)pyrrolidin-3-yl)acrylamide (Compound 911)

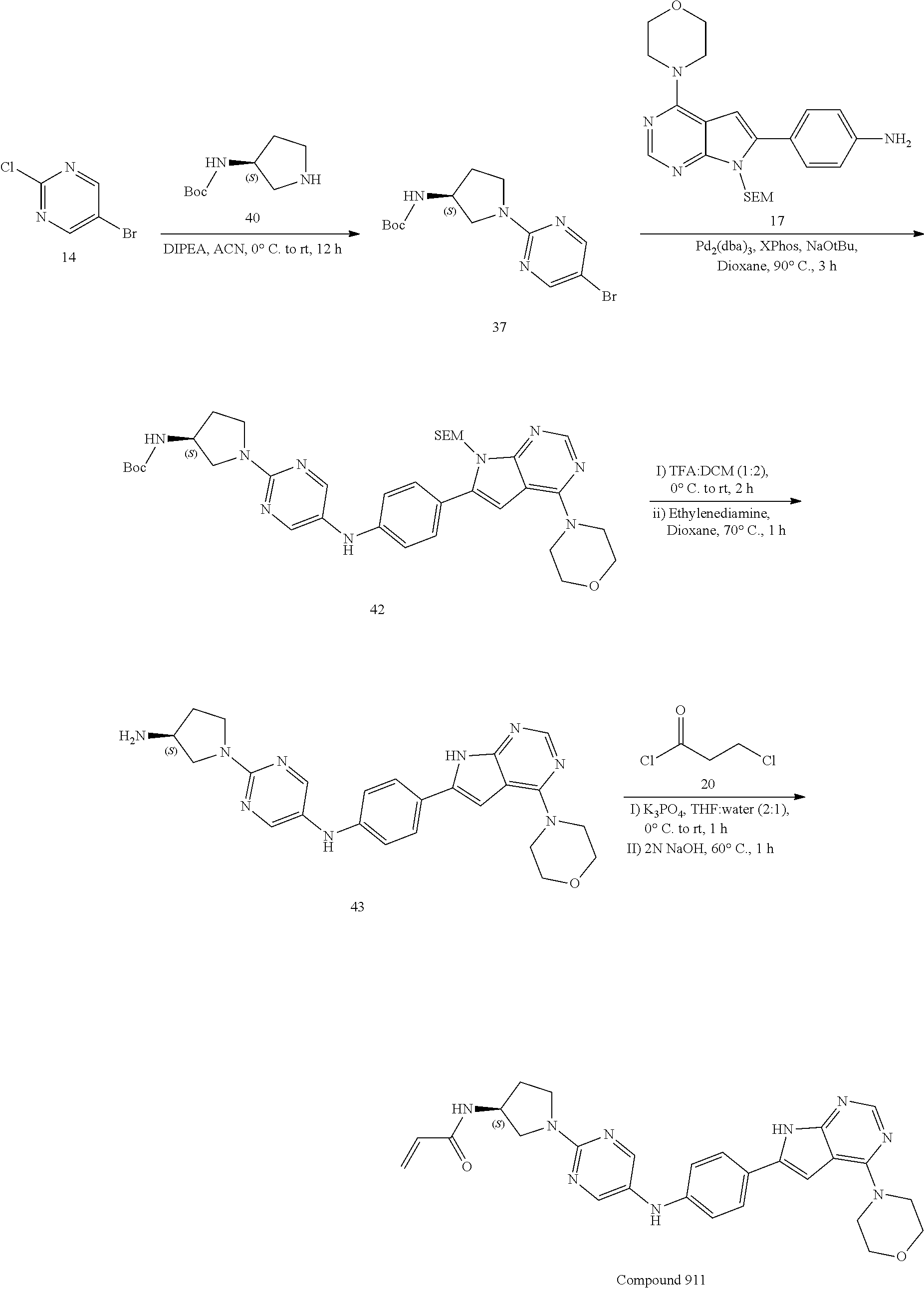

### Synthesis of tert-butyl(S)-(1-(5-bromopyrimidin-2-yl)pyrrolidin-3-yl)carbamate (41)

To a stirred solution of 5-bromo-2-chloropyrimidine 14 (1 g, 5.17 mmol) in ACN(10 mL) were added tert-butyl(S)-pyrrolidin-3-ylcarbamate 40 (770 mg, 4.14 mmol) and DIPEA (2.2 mL, 12.4 mmol) at 0° C. and stirred at RT for 12 h. After completion of reaction (monitoring by TLC), the reaction mixture was concentrated under reduce pressure. The crude was diluted with water (50 mL) and extracted with EtOAc (3×50 mL). The combined organic layer was washed with ice cold brine (2×50 mL), dried over Na₂SO₄, filtered and evaporated under reduced pressure. The crude was purified by flash chromatography using 10-15% EtOAc in heptane to get the titled compound as an off white solid 41 (1 g, 51%).

LCMS=[M−H]: 341.32; Purity=70%.

### Synthesis of tert-butyl(S)-(1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)pyrrolidin-3-yl)carbamate (42)

To a stirred solution of tert-butyl(S)-(1-(5-bromopyrimidin-2-yl)pyrrolidin-3-yl)carbamate 41 (1 g, 2.91 mmol) in 1,4-dioxane (15 mL) were added 4-(4-morpholino-7-((2-(trimethylsilyl)ethoxy)methyl)-7H-pyrrolo[2,3-d]pyrimidin-6-yl) aniline 17 (1.24 g, 2.91 mmol) and NaO′Bu (560 mg, 5.83 mmol) at RT. The reaction mixture was degassed with N₂ gas for 10 min, followed by XPhos (278 mg, 0.583 mmol) and Pd₂(dba)₃ (236 mg, 0.291 mmol) were added to the reaction mixture and stirred at 90° C. for 3 h. After completion of reaction (TLC monitoring), the reaction mixture was diluted with water (50 mL) and extracted with EtOAc (2×100 mL). The combined organic layer was washed with brine solution (2×50 mL), dried over Na₂SO₄, filtered and concentrated under reduced pressure. The crude was purified by flash chromatography (silica gel 100-200 M, 24 g SNAP) using 30-40% EtOAc in heptane to get the title compound as a pale yellow solid 42 (1 g, 50%) LCMS=[M+H]⁺: 688.30; Purity=93%.

### Synthesis of (S)-2-(3-aminopyrrolidin-1-yl)-N-(4-(4-morpholino-7H-pyrrolo[2,3-d]pyrimidin-6-yl)phenyl)pyrimidin-5-amine (43)

To a stirred solution of tert-butyl(S)-(1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)pyrrolidin-3-yl)carbamate 42 (700 mg, 1.02 mmol) in DCM (8 mL) was added TFA (4 mL) dropwise at 0° C. and stirred at RT for 2 h. After consumed of starting material (monitoring by TLC), the reaction mixture was concentrated under reduced pressure. The crude was triturated with Et₂O and dried under reduced pressure to obtain the solid. The solid was dissolved in 1,4-dioxane (5 mL) and 1,2-ethanediamine (0.1 mL, 1.53 mmol) was added to the reaction mixture at RT and stirred at 70° C. for 1 h. After completion of reaction (LCMS monitoring), the reaction mixture was diluted water (50 mL) and extracted with 25% IPA in CHCl₃ (3×50 mL). The organic was dried over Na₂SO₄ and concentrated under reduced pressure to get the title compound as a pale yellow solid 43 (550 mg; crude) which was used in next step directly without further purification.

LCMS=[M+H]⁺: 458.24; Purity=80%.

### Synthesis of (S)—N-(1-(5-((4-(4-morpholino-7H-pyrrolo[2,3-d]pyrimidin-6-yl)phenyl)amino)pyrimidin-2-yl)pyrrolidin-3-yl)acrylamide (Compound 911)

To a stirred solution of(S)-2-(3-aminopyrrolidin-1-yl)-N-(4-(4-morpholino-7H-pyrrolo[2,3-d]pyrimidin-6-yl)phenyl)pyrimidin-5-amine 43 (550 mg, 1.2 mmol) in THF (6 mL) was added K₃PO₄ (510 mg, 2.4 mmol) dissolved in water (3 mL) at 0° C. Then, 3-chloropropanoyl chloride 20 (183 mg, 1.44 mmol) was added dropwise to the reaction mixture and stirred at RT for 1 h. After consumed of starting material (monitoring by TLC and LCMS), 2N NaOH (7.2 mL, 14.4 mmol) was added dropwise to the reaction mixture at 0° C. and stirred at 60° C. for 1 h. After completion of reaction (LCMS monitoring), the reaction mixture was diluted with water (50 mL) and extracted with 2-MeTHF (3×50 mL). The combined organic layer was dried over Na₂SO₄, filtered and concentrated under reduced pressure. The crude was purified by RP-HPLC [5-95% 0.1% TFA in Water/ACN; Column: Symmetry C18 (19*300) 7μ] to get the title Compound 911 (274 mg, 36%).

¹H NMR (400 MHz, DMSO-d₆): δ 12.68 (brs, 1H), 8.40 (d, J=6.4 Hz, 1H), 8.30-8.29 (m, 3H), 7.93 (brs, 1H), 7.72 (d, J=8.8 Hz, 2H), 7.13 (s, 1H), 6.79 (d, J=8.8 Hz, 2H), 6.27-6.20 (m, 1H), 6.11 (dd, J=17.2 Hz, 2.4 Hz, 1H), 5.60 (dd, J=10 Hz, 2.4 Hz, 1H), 4.45-4.40 (m, 1H), 3.94-3.91 (m, 4H), 3.80-3.78 (m, 4H), 3.75-3.71 (m, 1H), 3.62-3.57 (m, 2H), 3.44-3.40 (m, 1H), 2.25-2.16 (m, 1H), 1.96-1.89 (m, 1H).

LCMS=[M+H]⁺: 512.27; Purity=99.53%.

### Example 63

### Compound 912

### Preparation of (S)—N-(1-(5-((4-(4-morpholino-7H-pyrrolo[2,3-d]pyrimidin-6-yl)phenyl)amino)pyrimidin-2-yl)piperidin-3-yl)acrylamide (Compound 912)

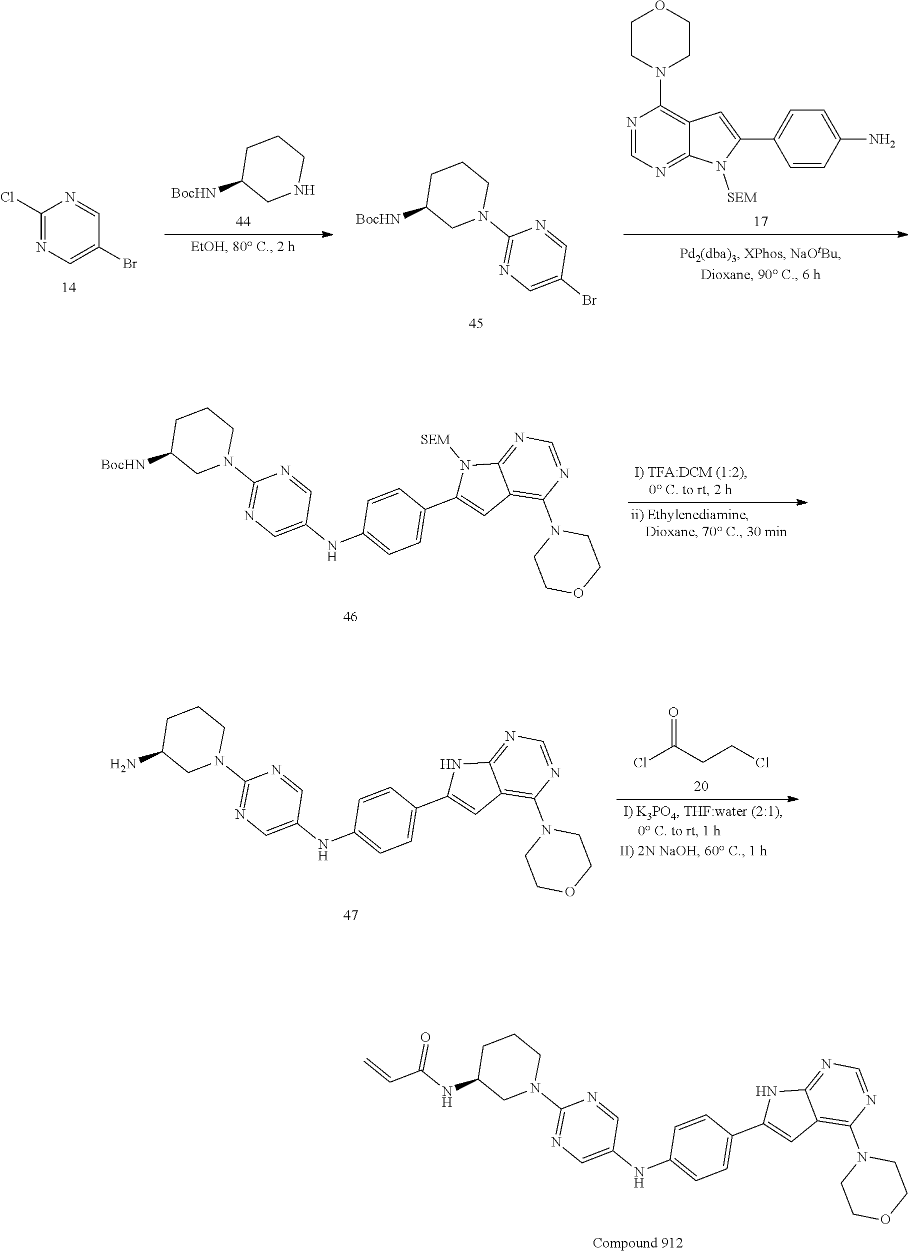

### Synthesis of tert-butyl(S)-(1-(5-bromopyrimidin-2-yl)piperidin-3-yl)carbamate (45)

To a stirred solution of 5-bromo-2-chloropyrimidine 14 (1 g, 5.17 mmol) in EtOH (20 mL) was added tert-butyl(S)-piperidin-3-ylcarbamate 44 (2.07 g, 10.3 mmol) at RT and stirred at 80° C. for 2 h. After completion of reaction (TLC monitoring), the reaction mixture was evaporated under reduced pressure. The crude was diluted with water (50 mL) and extracted with EtOAc (3×50 mL). The combined organic layer was dried over Na₂SO₄, filtered and evaporated under reduced pressure to get the desired compound as a brown solid 45 (1.5 g, 76%).

LCMS=[M+H]⁺: 359.04; Purity=93%.

### Synthesis of tert-butyl(S)-(1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl)carbamate (46)

To a stirred solution of tert-butyl(S)-(1-(5-bromopyrimidin-2-yl)piperidin-3-yl)carbamate 45 (1.5 g, 4.2 mmol) and 4-(4-morpholino-7-((2-(trimethylsilyl)ethoxy)methyl)-7H-pyrrolo[2,3-d]pyrimidin-6-yl) aniline 17 (1.79 g, 4.2 mmol) in 1,4-dioxane (20 mL) was added NaO′Bu (605 mg, 6.3 mmol) at RT. The reaction mixture was degassed with N₂ gas for 10 min, followed by XPhos (200 mg, 0.420 mmol) and Pd₂(dba)₃ (340 mg, 0.420 mmol) were added to the reaction mixture and stirred at 90° C. for 6 h. After completion of reaction (TLC and LCMS monitoring), the reaction mixture was diluted with water (50 mL) and extracted with EtOAc (3×50 mL). The combined organic layer was dried over Na₂SO₄, filtered and evaporated under reduced pressure. The crude was purified by flash chromatography (silica gel 100-200 M, 12 g SNAP) using 50-55% EtOAc in heptane to get the title compound as an off white solid 46 (3 g, 71%).

LCMS=[M+H]⁺: 702.61; Purity=70%

### Synthesis of (S)-2-(3-aminopiperidin-1-yl)-N-(4-(4-morpholino-7H-pyrrolo[2,3-d]pyrimidin-6-yl)phenyl)pyrimidin-5-amine (47)

To a stirred solution of tert-butyl(S)-(1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl)carbamate 46 (3 g, 4.27 mmol) in DCM (20 mL) was added TFA (10 mL) dropwise at 0° C. and stirred at RT for 2 h. After consumed of starting material (monitoring by TLC), the reaction mixture was concentrated under reduced pressure. The crude was dissolved in 1,4-dioxane (10 mL) and 1,2-ethanediamine (0.86 mL, 12.8 mmol) was added to the reaction mixture at RT and stirred at 70° C. for 30 min. After completion of reaction (LCMS monitoring), the reaction mixture was diluted with ice cold water (100 mL) and extracted with 25% IPA in CHCl₃ (3×100 mL). The combined organic layer was dried over Na₂SO₄, filtered and concentrated under reduced pressure to get the title compound as a white solid 47 (2 g; crude) which was used in next step directly without further purification.

LCMS=[M+H]⁺: 472.44; Purity=70%.

### Synthesis of (S)—N-(1-(5-((4-(4-morpholino-7H-pyrrolo[2,3-d]pyrimidin-6-yl)phenyl)amino)pyrimidin-2-yl)piperidin-3-yl)acrylamide (Compound 912)

To a stirred solution of(S)-2-(3-aminopiperidin-1-yl)-N-(4-(4-morpholino-7H-pyrrolo[2,3-d]pyrimidin-6-yl)phenyl)pyrimidin-5-amine 47 (1 g, 2.12 mmol) in THF (20 mL) was added K₃PO₄ (1.35 g, 6.36 mmol) dissolved in water (10 mL) at 0° C. under N₂ atmosphere. Then, 3-chloropropanoyl chloride 20 (0.25 mL, 2.54 mmol) was added dropwise to the reaction mixture at 0° C. and stirred at RT for 1 h. After consumed of starting material (monitoring by TLC and LCMS), 2N NaOH (12.7 mL, 25.4 mmol) was added to the reaction mixture dropwise at 0° C. and stirred at 60° C. for 1 h. After completion of reaction (LCMS monitoring), the reaction mixture was diluted with water (100 mL) and extracted with 2-MeTHF (3×100 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get the title compound Compound 912 (147 mg, 13%).

¹H NMR (400 MHz, DMSO-d₆): δ 12.02 (s, 1H), 8.28 (s, 2H), 8.13 (s, 1H), 8.11 (d, J=8 Hz, 1H), 7.89 (s, 1H), 7.70 (d, J=8.8 Hz, 2H), 6.91 (d, J=1.6 Hz, 1H), 6.80 (d, J=8.4 Hz, 2H), 6.28-6.21 (m, 1H), 6.10 (dd, J=16.8 Hz, 2 Hz, 1H), 5.59 (dd, J=10 Hz, 2.4 Hz, 1H), 4.45-4.41 (m, 1H), 4.30-4.27 (m, 1H), 3.86-3.83 (m, 4H), 3.80-3.72 (m, 5H), 3.08 (t, J=9.6 Hz, 1H), 2.96-2.92 (m, 1H), 1.95-1.90 (m, 1H), 1.80-1.75 (m, 1H), 1.54-1.44 (m, 2H).

LCMS=[M+H]⁺: 526.25; Purity=99.82%.

### Example 64

### Compound 913

### Preparation of N-(1-(5-((4-(4-morpholino-7H-pyrrolo[2,3-d]pyrimidin-6-yl)phenyl)amino)pyrimidin-2-yl) azetidin-3-yl)acrylamide (Compound 913)

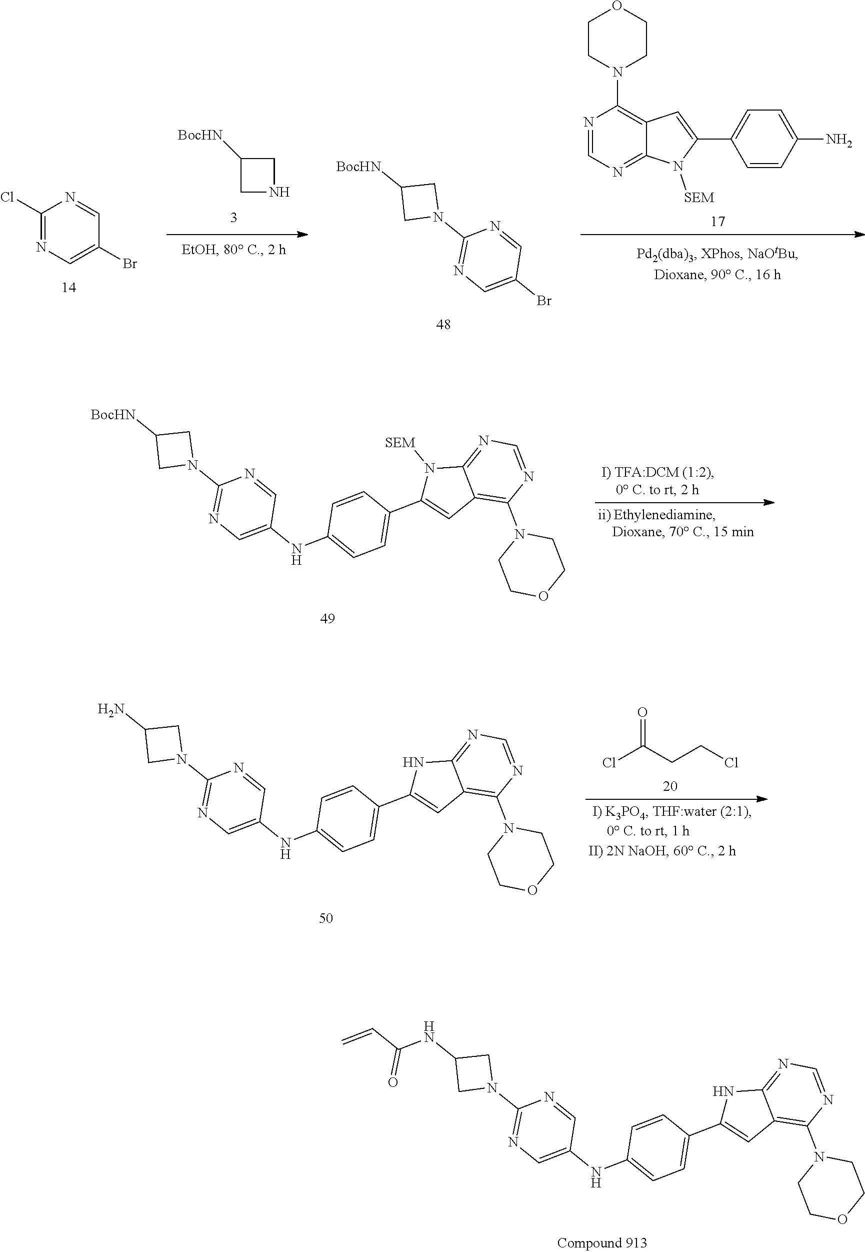

### Synthesis of tert-butyl (1-(5-bromopyrimidin-2-yl)azetidin-3-yl)carbamate (48)

To a stirred solution of 5-bromo-2-chloropyrimidine 14 (800 mg, 4.14 mmol) in EtOH (10 mL) was added tert-butyl 3-azetidinylcarbamate 3 (712 mg, 4.14 mmol) at RT under N₂ atmosphere and stirred at 80° C. for 2 h. After completion of reaction (monitoring by TLC), the reaction mixture was concentrated under reduced pressure. The crude was diluted with water (50 mL) and extracted with EtOAc (3×50 mL). The combined organic layer was washed with brine solution (50 mL), dried over Na₂SO₄, filtered and concentrated under reduced pressure to get the title compound as a pale yellow solid 48 (700 mg, 51%).

LCMS=[M+H]⁺: 329.08; Purity=75%

### Synthesis of tert-butyl (1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)azetidin-3-yl)carbamate (49)

To a stirred solution of tert-butyl [1-(5-bromo-2-pyrimidinyl)-3-azetidinyl]carbamate 48 (700 mg, 2.13 mmol) in 1,4-dioxane (10 mL) were added 4-(4-morpholino-7-((2-(trimethylsilyl)ethoxy)methyl)-7H-pyrrolo[2,3-d]pyrimidin-6-yl)aniline 17 (905 mg, 2.13 mmol) and NaO′Bu (307 mg, 3.19 mmol) sequentially at RT. The reaction mixture was degassed with N₂ gas for 10 min, followed by XPhos (101 mg, 0.213 mmol) and Pd₂(dba)₃ (172 mg, 0.213 mmol) were added to the reaction mixture and stirred at 90° C. for 16 h. After completion of reaction (TLC and LCMS monitoring), the reaction mixture was filtered through Celite pad and washed with EtOAc. The filtrate was diluted with water (50 mL) and extracted with EtOAc (3×50 mL). The combined organic layer was washed with brine (2×50 mL), dried over Na₂SO₄, filtered and evaporated under reduced pressure. The crude was purified by flash chromatography using 40-50% EtOAc in heptane to get the desired product as a yellow solid 49 (400 mg, 28%).

LCMS=[M+H]⁺: 674.65; Purity=64%.

### Synthesis of 2-(3-aminoazetidin-1-yl)-N-(4-(4-morpholino-7H-pyrrolo[2,3-d]pyrimidin-6-yl)phenyl)pyrimidin-5-amine (50)

To a stirred solution of tert-butyl (1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)azetidin-3-yl)carbamate 49 (400 mg, 0.594 mmol) in DCM (5 mL) was added TFA (2.5 mL, 52.3 mmol) dropwise at 0° C. and stirred at RT for 2 h. After consumed of starting material (monitoring by TLC), the reaction mixture was concentrated under reduced pressure. The crude was dissolved in 1,4-dioxane (5 mL) and 1,2-ethanediamine (59.5 μL, 0.89 mmol) was added at RT and stirred at 70° C. for 15 min. After completion of reaction (LCMS monitoring), the reaction mixture was diluted with 25% IPA in CHCl₃ (50 mL) and washed with water (2×30 mL). The organic layer was dried over Na₂SO₄, filtered and concentrated under reduced pressure to get the desired compound as a yellow solid 50 (250 mg; crude) which was used in next step directly without further purification.

LCMS=[M+H]⁺: 444.44; Purity=59%.

### Synthesis of N-(1-(5-((4-(4-morpholino-7H-pyrrolo[2,3-d]pyrimidin-6-yl)phenyl)amino)pyrimidin-2-yl)azetidin-3-yl)acrylamide (Compound 913)

To a, stirred solution of 2-(3-amino-1-azetidinyl)-5-[α-(4-morpholino-1H-1,5,7-triazainden-2-yl)phenylamino]pyrimidine 50 (250 mg, 0.564 mmol) in THF (5 mL) was added K₃PO₄ (598 mg, 2.82 mmol) dissolved in water (2.5 mL) at 0° C. Then, 3-chloropropionyl chloride 20 (64.6 μL, 676 mmol) was dissolved in THF (1 mL) added dropwise to the reaction mixture at 0° C. and stirred at RT for 1 h. After consumed of starting material (monitoring by TLC), 2N NaOH (3.5 mL, 6.76 mmol) was added dropwise at 0° C. and stirred at 60° C. for 2 h. After completion of reaction (LCMS monitoring), the reaction mixture was diluted with water (50 mL) and extracted with 2-MeTHF (3×50 mL). The combined organic layer was dried over Na₂SO₄, filtered and concentrated under reduced pressure. The crude was purified by RP-HPLC [2-98% 5 mM ammonium bicarbonate in water/ACN; Column: Symmetry C18 (19*300) 7μ] to get desired Compound 913 (52 mg, 18%). 1H NMR (400 MHz, DMSO-d₆): δ 12.02 (s, 1H), 8.80 (d, J=6.8 Hz, 1H), 8.31 (s, 2H), 8.14 (s, 1H), 7.94 (s, 1H), 7.71 (d, J=8.8 Hz, 2H), 6.92 (d, J=2 Hz, 1H), 6.81 (d, J=8.8 Hz, 2H), 6.25-6.21 (m, 1H), 6.12 (dd, J=17.2 Hz. 2.4 Hz, 1H), 5.64 (dd, J=9.6 Hz, 2.4 Hz, 1H), 4.70-4.66 (m, 1H), 4.30 (t, J=8 Hz, 2H), 3.90-3.79 (m, 6H), 3.74-3.72 (m, 4H).

LCMS=[M+H]⁺: 498.19; Purity=98.17%.

### Example 65

### Compound 914

### Preparation of (R)—N-(1-(4-methyl-5-((4-(4-morpholino-7H-pyrrolo[2,3-d]pyrimidin-6-yl)phenyl)amino)pyrimidin-2-yl)piperidin-3-yl)acrylamide (Compound 914)

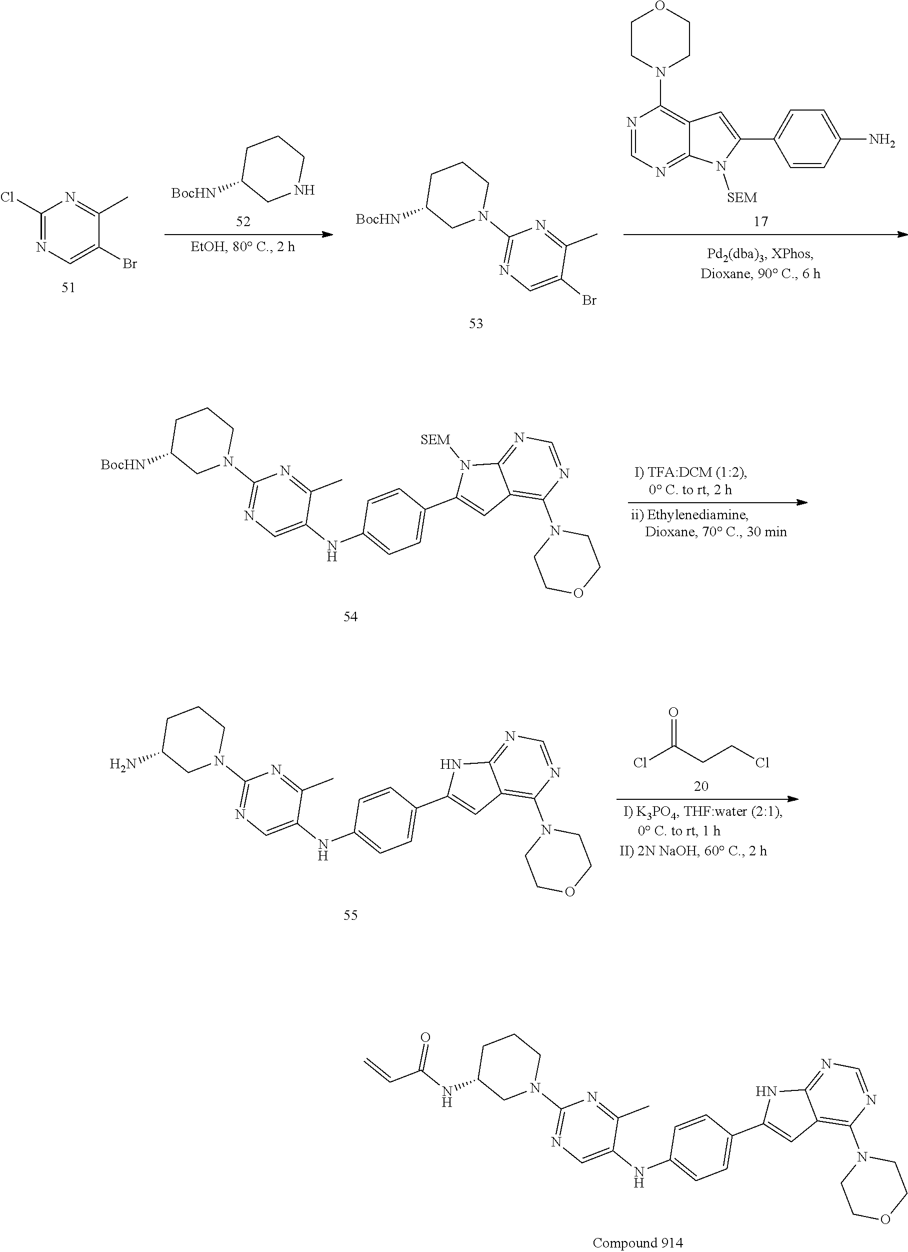

### Synthesis of tert-butyl (R)-(1-(5-bromo-4-methylpyrimidin-2-yl)piperidin-3-yl)carbamate (53)

To a stirred solution of 5-bromo-2-chloro-4-methylpyrimidine 51 (1 g, 4.82 mmol) in in EtOH (10 mL) was added tert-butyl (R)-piperidin-3-ylcarbamate 52 (1.66 g, 8.27 mmol) at RT and stirred at 80° C. for 2 h. After completion of reaction (TLC monitoring), the reaction mixture was evaporated under reduced pressure. The crude was diluted with water (50 mL) and extracted with EtOAc (3×50 mL). The combined organic layer was dried over Na₂SO₄, filtered and evaporated under reduced pressure to get the title compound as a brown solid 53 (1.4 g, 91%).

LCMS=[M+H]⁺: 371.26; Purity: 66%

### Synthesis of tert-butyl (R)-(1-(4-methyl-5-((4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl)carbamate (54)

To a stirred solution of tert-butyl (R)-(1-(5-bromo-4-methylpyrimidin-2-yl)piperidin-3-yl)carbamate 53 (1 g, 2.69 mmol) in 1,4-dioxane (20 mL) added 4-(4-morpholino-7-((2-(trimethylsilyl)ethoxy)methyl)-7H-pyrrolo[2,3-d]pyrimidin-6-yl)aniline 17 (1.15 g, 2.69 mmol) and NaOtBu (518 mg, 5.39 mmol). The reaction mixture was degassed with N₂ gas for 10 min, followed by XPhos (257 mg, 0.539 mmol) and Pd₂(dba)₃ (218 mg, 0.269 mmol) were added to the reaction mixture and stirred at 90° C. for 6 h. After completion of reaction (TLC monitoring), the reaction mixture was diluted with water (50 mL) and extracted with EtOAc (3×50 mL). The combined organic layer was washed with brine solution (2×50 mL), dried over Na₂SO₄, filtered and concentrated under reduced pressure. The crude was purified by flash chromatography (silica gel 100-200 M, 12 g SNAP) using 25-30% EtOAc in heptane to get the title compound as light yellow solid 54 (1 g, 52%)

LCMS=[M+H]⁺: 716.44; Purity=84%

### Synthesis of (R)-2-(3-aminopiperidin-1-yl)-4-methyl-N-(4-(4-morpholino-7H-pyrrolo[2,3-d]pyrimidin-6-yl)phenyl)pyrimidin-5-amine (55)

To a stirred solution of tert-butyl (R)-(1-(4-methyl-5-((4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3 -yl)carbamate 54 (1 g, 1.4 mmol) in DCM (10 mL) was added TFA (5 mL) dropwise at 0° C. and stirred at RT for 2 h. After completion of reaction (TLC monitoring), the reaction mixture was concentrated under reduced pressure. The crude was dissolved in 1,4-dioxane (10 mL) and 1,2-ethanediamine (0.14 mL, 2.1 mmol) was added at RT and stirred at 70° C. for 30 min. After completion of reaction (LCMS monitoring), the reaction mixture was diluted with 25% IPA in CHCl₃ (50 mL) and washed with ice cold water (2×50 mL). The organic layer was dried over Na₂SO₄, filtered and concentrated under reduced pressure to get the title compound as solid an off white solid 55 (800 mg; crude) which was used in next step directly without further purification.

LCMS=[M+H]⁺: 486.46; Purity=57%

### Synthesis of (R)—N-(1-(4-methyl-5-((4-(4-morpholino-7H-pyrrolo[2,3-d]pyrimidin-6-yl)phenyl)amino)pyrimidin-2-yl)piperidin-3-yl)acrylamide (Compound 914)

To a stirred solution of (R)-2-(3-aminopiperidin-1-yl)-4-methyl-N-(4-(4-morpholino-7H-pyrrolo[2,3-d]pyrimidin-6-yl)phenyl)pyrimidin-5-amine 55 (500 mg, 1.03 mmol) in THF (5 mL) was added K₃PO₄ (437 mg, 2.06 mmol) dissolved in water (2.5 mL) at 0° C. Then, 3-chloropropionyl chloride 20 (157 mg, 1.24 mmol) was added dropwise to the reaction mixture at 0° C. and stirred at RT for 1 h. After consumed of starting material (monitoring by TLC), 2N aq. NaOH (6.18 mL, 12.4 mmol) was added dropwise to the reaction mixture at 0° C. and stirred at 60° C. for 2 h. After completion of reaction (LCMS monitoring), the reaction mixture was diluted with ice cold water (50 mL) and extracted with 2-MeTHF (3×50 mL). The combined organic layer was dried over Na₂SO₄, filtered and concentrated under reduced pressure. The crude was purified by RP-HPLC [5-95% 0.1% TFA in Water/ACN; [Column: X Bridge C8 (19*250) 5μ] to get the title compound Compound 914 (181 mg, 33%).

¹H NMR (400 MHz, DMSO-d₆): δ 12.70 (brs, 1H), 8.29 (s, 1H), 8.13-8.10 (m, 2H), 7.69 (d, J=8.8 Hz, 2H), 7.64 (s, 1H), 7.10 (s, 1H), 6.61 (d, J=8.4 Hz, 2H), 6.25 (dd, J=16.8 Hz, 10 Hz, 1H), 6.10 (dd, J=17.2 Hz, 2 Hz, 1H), 5.59 (dd, J=10 Hz, 2 Hz, 1H), 4.47 (d, J=9.6 Hz, 1H), 4.34 (d, J=12.4 Hz, 1H), 3.93-3.91 (m, 4H), 3.80-3.75 (m, 5H), 3.07 (t, J=10 Hz, 1H), 2.92 (q, J=9.2 Hz, 1H), 2.19 (s, 3H), 1.95-1.91 (m, 1H), 1.78-1.70 (m, 1H), 1.51-1.46 (m, 2H).

LCMS=[M+H]: 540.28; Purity=98.42%.

### Example 66

### Compound 915

### Preparation of (R)-2-(azetidin-1-ylmethyl)-N-(1-(5-((4-(4-morpholino-7H-pyrrolo[2,3-d]pyrimidin-6-yl)phenyl)amino)pyrimidin-2-yl)piperidin-3-yl)acrylamide (Compound 915)

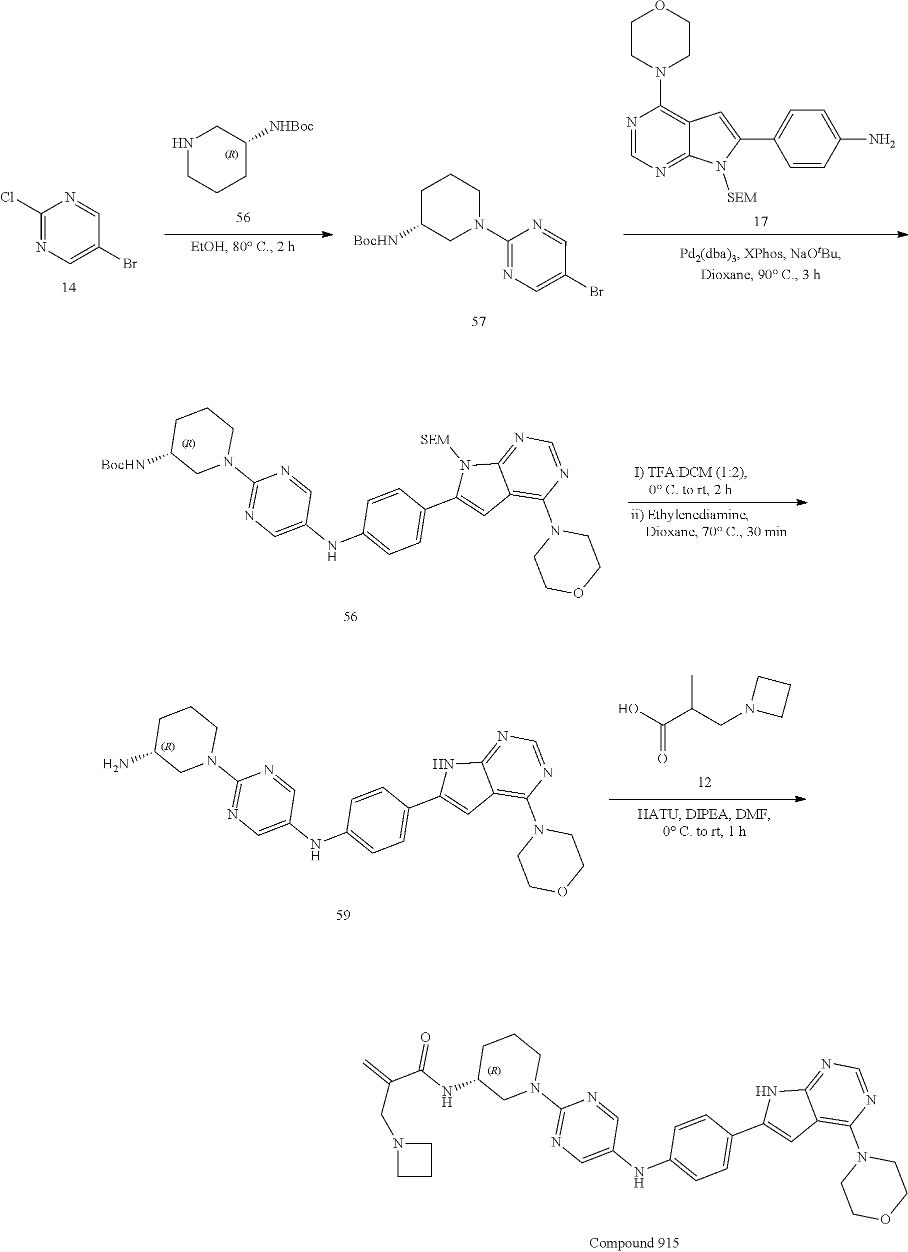

### Synthesis of tert-butyl (R)-(1-(5-bromopyrimidin-2-yl)piperidin-3-yl)carbamate (57)

To a stirred solution of 5-bromo-2-chloropyrimidine 14 (800 mg, 4.14 mmol) in EtOH (10 mL) was added (R)-3-piperidylamino-tert-butylformylate 56 (1.66 g, 8.27 mmol) at RT and stirred at 80° C. for 2 h. After completion of reaction (TLC monitoring), the reaction mass was evaporated under reduced pressure. The crude was diluted with water (50 mL) and extracted with EtOAc (3×50 mL). The combined organic layer was dried over Na₂SO₄, filtered and evaporated under reduced pressure to get the title compound as a brown solid 57 (1.4 g, 95%).

LCMS=[M+H]⁺: 359.35; Purity: 99.37%

### Synthesis of tert-butyl (R)-(1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl)carbamate (58)

To a stirred solution of tert-butyl (R)-(1-(5-bromopyrimidin-2-yl)piperidin-3-yl)carbamate 57 (1.4 g, 3.92 mmol) in 1,4-dioxane (20 mL) were added 4-(4-morpholino-7-((2-(trimethylsilyl)ethoxy)methyl)-7H-pyrrolo[2,3-d]pyrimidin-6-yl)aniline 17 (1.67 g, 3.92 mmol) and NaOtBu (565 mg, 5.88 mmol). The reaction mixture was degassed with N₂ gas for 10 min, followed by XPhos (187 mg, 0.392 mmol) and Pd₂(dba)₃ (317 mg, 0.392 mmol) were added to the reaction mixture and stirred at 90° C. for 3 h. After completion of reaction (TLC and LCMS monitoring), the reaction mixture was diluted with water (100 mL) and extracted with EtOAc (3×100 mL). The combined organic layer was dried over Na₂SO₄, filtered and evaporated under reduced pressure. The crude was purified by flash chromatography (silica gel 100-200 M, 40 g SNAP) using 50-55% EtOAc in heptane to get the desired compound as an off white solid 58 (1.5 g, 38%). LCMS=[M+H]⁺: 702.50; Purity=70%.

### Synthesis of (R)-2-(3-aminopiperidin-1-yl)-N-(4-(4-morpholino-7H-pyrrolo[2,3-d]pyrimidin-6-yl)phenyl)pyrimidin-5-amien (59)

To a stirred solution of tert-butyl (R)-(1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl)carbamate 58 (1.5 g, 2.14 mmol) in DCM (10 mL) was added TFA (5 mL) dropwise at 0° C. and stirred at RT for 2 h. After consumed of starting material (monitoring by TLC), the reaction mixture was concentrated under reduced pressure, triturated with Et2O and dried under reduced pressure. The crude was dissolved in 1,4-dioxane (10 mL) and 1,2-ethanediamine (0.43 mL, 6.41 mmol) was added at RT and stirred at 70° C. for 30 min. After completion of reaction (LCMS monitoring), the reaction mixture was diluted with 25% IPA in CHCl₃ (100 mL) and washed with ice cold water (2×50 mL). The organic layer was dried over Na₂SO₄, filtered and concentrated under reduced pressure to get the title compound as a yellow solid 59 (1 g; crude) which was used in next step directly without further purification.

LCMS=[M+H]⁺: 472.40; Purity=60%.

### Synthesis of (R)-2-(azetidin-1-ylmethyl)-N-(1-(5-((4-(4-morpholino-7H-pyrrolo[2,3-d]pyrimidin-6-yl)phenyl)amino)pyrimidin-2-yl)piperidin-3-yl)acrylamide (Compound 915)

To a stirred solution of (R)-2-(3-aminopiperidin-1-yl)-N-(4-(4-morpholino-7H-pyrrolo[2,3-d]pyrimidin-6-yl)phenyl)pyrimidin-5-amien 59 (500 mg, 1.06 mmol) in DMF (5 mL) were added 2-(azetidin-1-ylmethyl)acrylic acid 12 (225 mg, 1.59 mmol), DIPEA (0.56 mL, 3.18 mmol) and HATU (605 mg, 1.59 mmol) sequentially at 0° C. and stirred at RT for 1 h. After completion of reaction (TLC and LCMS monitoring), the reaction mixture was poured into ice cold water (50 mL) and extracted with 25% IPA in CHCl₃ (3×50 mL). The combined organic layer was washed with ice cold brine solution (3×50 mL), dried over Na₂SO₄, filtered and concentrated under reduced pressure. The crude was purified by RP-HPLC [5-95% 5 mM ammonium bicarbonate in water/ACN; Column: X Bridge C8 (19*250) 5μ] to get the title compound Compound 915 (19 mg, 3%).

¹H NMR (400 MHz, DMSO-d₆): δ 12.00 (s, 1H), 9.11 (d, J=7.6 Hz, 1H), 8.27 (s, 2H), 8.13 (s, 1H), 7.87 (s, 1H), 7.69 (d, J=8.8 Hz, 2H), 6.91 (d, J=1.6 Hz, 1H), 6.76 (d, J=8.8 Hz, 2H), 5.80 (d, J=1.6 Hz, 1H), 5.38 (s, 1H), 3.89-3.83 (m, 6H), 3.77-3.68 (m, 8H), 3.11 (q, J=12.8 Hz, 2H), 3.02 (q, J=6.8 Hz, 2H), 2.94 (q, J=6.8 Hz, 2H), 1.90-1.84 (m, 3H), 1.70-1.64 (m, 2H).

LCMS=[M+H]⁺: 595.38; Purity=99.70%.

### Example 67

### Compound 916

### (R)-4-((3-(2-(azetidin-1-ylmethyl)acrylamido)piperidin-1-yl)methyl)-N-(4-(4-morpholino-7H-pyrrolo[2,3-d]pyrimidin-6-yl)phenyl)picolinamide (Compound 916)

Step 1:2-(azetidin-1-ylmethyl)acrylic acid

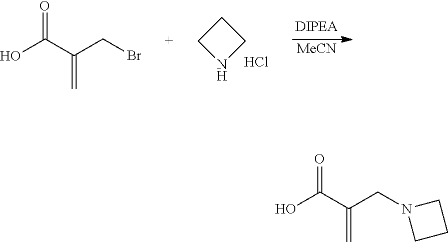

A solution of 2-(bromomethyl)prop-2-enoic acid (250 mg, 1.52 mmol, azetidine hydrochloride (184 mg, 1.97 mmol), and N,N-diisopropylethylamine (0.79 mL, 4.55 mmol) in acetonitrile (4 mL) was stirred at 35° C. for 20 hours, providing a solution of 2-(azetidin-1-ylmethyl)acrylic acid (213 mg, 1.51 mmol, 99% yield) in acetonitrile. The solution was used in the next step without workup.

Step 2: (R)-4-((3-(2-(azetidin-1-ylmethyl)acrylamido)piperidin-1-yl)methyl)-N-(4-(4-morpholino-7H-pyrrolo[2,3-d]pyrimidin-6-yl)phenyl)picolinamide (Compound 916)

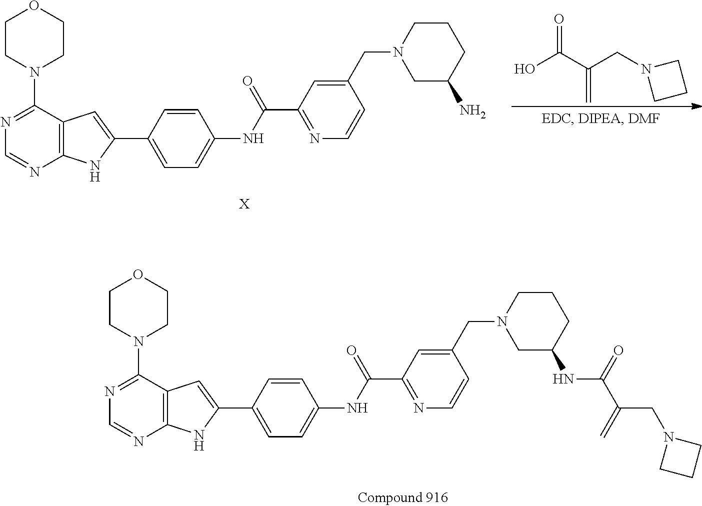

(R)-4-((3-aminopiperidin-1-yl)methyl)-N-(4-(4-morpholino-7H-pyrrolo[2,3-d]pyrimidin-6-yl)phenyl)picolinamide trihydrochloride (50 mg, 0.0804 mmol) was added to a solution of 1-(3-dimethylaminopropyl)-3-ethylcarbodiimide hydrochloride (23 mg, 0.121 mmol), 2-(azetidin-1-ylmethyl)acrylic acid (17 mg, 0.121 mmol)(as a solution in acetonitrile), and N,N-diisopropylethylamine (0.11 mL, 0.643 mmol) in N,N-dimethylformamide (4 mL). After 16 hours at 32° C. the reaction was subjected to preparative HPLC(5-85% acetonitrile/water, 0.1% trifluoroacetic acid). The fractions containing product were combined and subjected to lyophilization, providing, Compound 916 (12 mg, 0.0155 mmol, 19% yield) ¹H NMR (400 MHz, MeOD) δ 8.87 (d, J=4.9 Hz, 1H), 8.45-8.40 (m, 1H), 8.33 (s, 1H), 8.04-7.95 (m, 2H), 7.94-7.87 (m, 2H), 7.80 (dd, J=5.0, 1.7 Hz, 1H), 7.30 (s, 1H), 6.26 (s, 1H), 6.04 (s, 1H), 4.53 (s, 2H), 4.31-4.15 (m, 3H), 4.11 (m, 6H), 4.02 (m, 2H), 3.95 (m, 4H), 3.52 (m, 1H), 3.47 (m, 1H), 3.23-2.88 (m, 2H), 2.57 (m, 1H), 2.42 (m, 1H), 2.19-2.01 (m, 2H), 2.00-1.86 (m, 1H), 1.72 (m, 1H).

LCMS=[M+H]⁺: 636.4; Purity=95%.

### Example 68

### Compound 917

### Preparation of (R)-4-((3-acrylamidopiperidin-1-yl)methyl)-N-(5-(4-morpholino-7H-pyrrolo[2,3-d]pyrimidin-6-yl)pyrazin-2-yl)picolinamide (Compound 917)

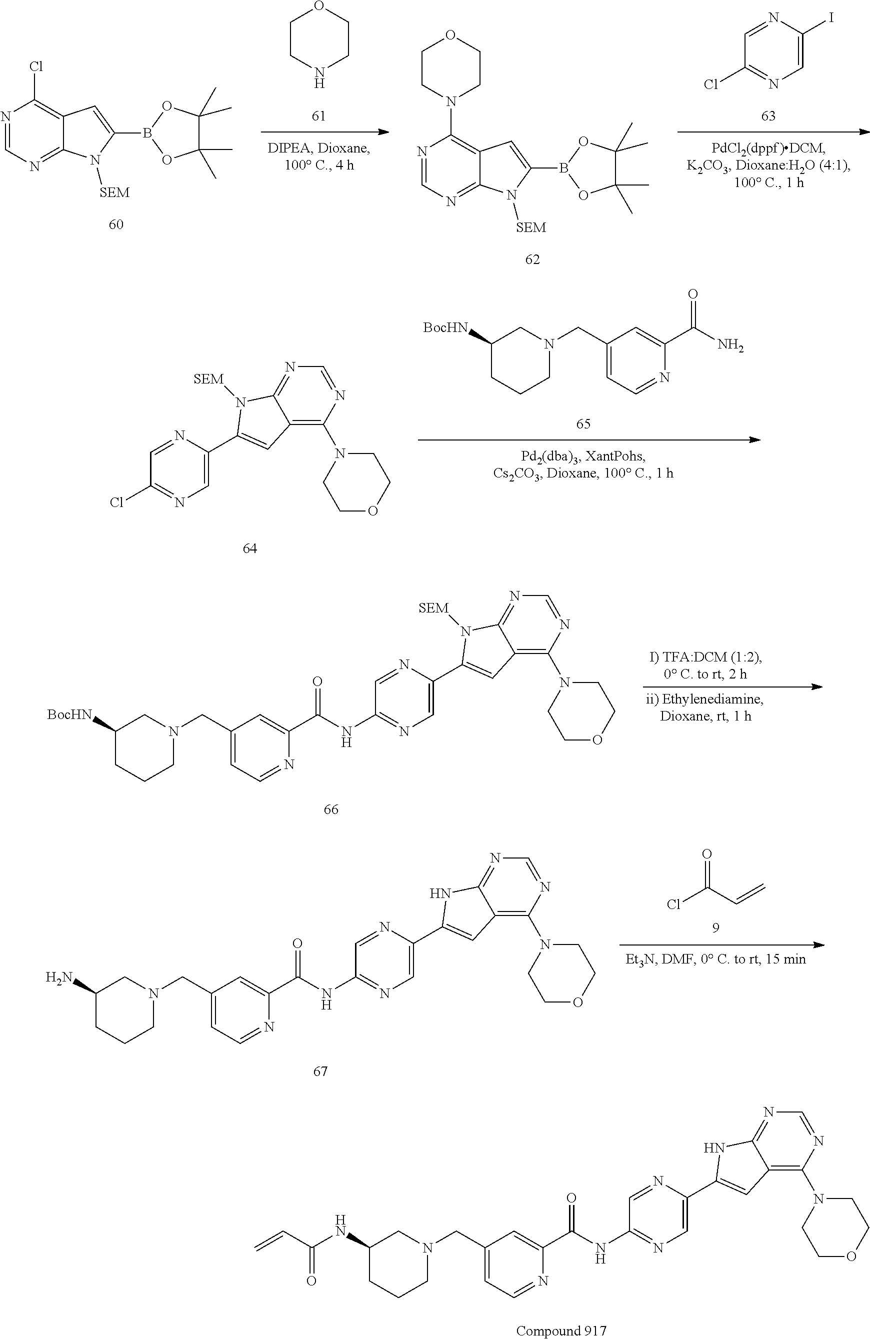

### Synthesis of 4-(6-(4,4,5,5-tetramethyl-1,3,2-dioxaborolan-2-yl)-7-((2-(trimethylsilyl)ethoxy)methyl)-7H-pyrrolo[2,3-d]pyrimidin-4-yl)morpholine (62)

To a stirred solution of 4-chloro-6-(4,4,5,5-tetramethyl-1,3,2-dioxaborolan-2-yl)-7-((2-(trimethylsilyl)ethoxy)methyl)-7H-pyrrolo[2,3-d]pyrimidine 60 (1 g, 2.44 mmol) in 1,4-dioxane (10 mL) were added morpholine 61 (0.25 mL, 2.93 mmol) and DIPEA (1.28 mL, 7.32 mmol) at RT and stirred at 100° C. for 4 h. After completion of reaction (TLC and LCMS monitoring), the reaction mixture was diluted with water (50 mL) and extracted with 25% IPA in CHCl₃ (3×50 mL). The combined organic layer was washed with brine (2×50 mL), dried over Na₂SO₄, filtered and concentrated under reduced pressure to get the title compound as brown liquid (1 g; crude) which was forwarded to next step without any purification.

LCMS=[M+H]⁺: 461.39; Purity=70%

### Synthesis of 4-(6-(5-chloropyrazin-2-yl)-7-((2-(trimethylsilyl)ethoxy)methyl)-7H-pyrrolo[2,3-d]pyrimidin-4-yl)morpholine (64)

To a stirred solution of 2-chloro-5-iodopyrazine 63 (300 mg, 1.25 mmol) in 1,4-dioxane (8 mL) and water (2 mL) were added 4-(6-(4,4,5,5-tetramethyl-1,3,2-dioxaborolan-2-yl)-7-((2-(trimethylsilyl)ethoxy)methyl)-7H-pyrrolo[2,3-d]pyrimidin-4-yl)morpholine 62 (575 mg, 1.25 mmol) and K₂CO₃ (517 mg, 3.74 mmol) at RT. The reaction mixture was degassed with N₂ gas for 15 min, followed by PdCl₂(dppf)·DCM (91.2 mg, 0.125 mmol) was added to the reaction mixture and stirred at 100° C. for 1 h. After completion of reaction (TLC and LCMS monitoring), the reaction mixture was filtered through Celite pad and washed with EtOAc. The filtrate was diluted with water (50 mL) and extracted with EtOAc (3×50 mL). The combined organic layer was dried over Na₂SO₄, filtered and evaporated under reduced pressure. The crude was purified by flash chromatography (silica gel 100-200 M, 12 g SNAP) using 40-45% EtOAc in heptane to get the title compound as an off yellow solid 64 (500 mg, 89%).

LCMS=[M+H]⁺: 447.29; Purity=70%

### Synthesis of tert-butyl (R)-(1-((2-((5-(4-morpholino-7-((2-(trimethylsilyl)ethoxy)methyl)-7H-pyrrolo[2,3-d]pyrimidin-6-yl)pyrazin-2-yl)carbamoyl)pyridin-4-yl)methyl)piperidin-3-yl)carbamate (66)

To a stirred solution of 4-(6-(5-chloropyrazin-2-yl)-7-((2-(trimethylsilyl)ethoxy)methyl)-7H-pyrrolo[2,3-d]pyrimidin-4-yl)morpholine 64 (500 mg, 1.12 mmol) in 1,4-dioxane (10 mL) were added tert-butyl (R)-(1-((2-carbamoylpyridin-4-yl)methyl)piperidin-3-yl)carbamate 65 (449 mg, 1.34 mmol) and Cs₂CO₃ (1.09 g, 3.36 mmol) at RT. The reaction mixture was degassed with N₂ gas for 10 min, followed by Pd₂(dba)₃ (90.5 mg, 0.112 mmol) and XantPhos (129 mg, 0.224 mmol) were added to the reaction mixture and stirred at 100° C. for 1 h. After completion of reaction (TLC and LCMS monitoring), the reaction mixture was diluted with water (50 mL) and extracted with EtOAc (3×50 mL). The combined organic layer was washed with brine solution (2×50 mL), dried over Na₂SO₄, filtered and concentrated under reduced pressure. The crude was purified by flash chromatography (silica gel 100-200 M, 12 g SNAP) using 60-70% EtOAc in heptane to get the title compound as a pale yellow solid 66 (350 mg, 42%).

LCMS=[M+H]⁺: 745.60; Purity=60%.

### Synthesis of (R)-4-((3-aminopiperidin-1-yl)methyl)-N-(5-(4-morpholino-7H-pyrrolo[2,3-d]pyrimidin-6-yl)pyrazin-2-yl)picolinamide (67)

To a stirred solution of tert-butyl (R)-(1-((2-((5-(4-morpholino-7-((2-(trimethylsilyl)ethoxy)methyl)-7H-pyrrolo[2,3-d]pyrimidin-6-yl)pyrazin-2-yl)carbamoyl)pyridin-4-yl)methyl)piperidin-3-yl)carbamate 66 (350 mg, 0.470 mmol) in DCM (5 mL) was added TFA (2.5 mL) dropwise at 0° C. and stirred at RT for 2 h. After consumed of starting material (monitoring by TLC), the reaction mixture was concentrated under reduced pressure. The crude was dissolved in 1,4-dioxane (5 mL) and 1,2-ethanediamine (17.9 μL, 0.268 mmol) was added to the reaction mixture and stirred at RT for 1 h. After completion of reaction (LCMS monitoring), the reaction mixture was diluted with water (20 mL) and extracted with 25% IPA in CHCl₃ (3×50 mL). The combined organic layer was dried over Na₂SO₄, filtered and concentrated under reduced pressure to get the title compound as a yellow solid 67 (200 mg; crude) which was used in next step directly without further purification. LCMS=[M+H]⁺: 515.41; Purity=67%

### Synthesis of (R)-4-((3-acrylamidopiperidin-1-yl)methyl)-N-(5-(4-morpholino-7H-pyrrolo[2,3-d]pyrimidin-6-yl)pyrazin-2-yl)picolinamide (Compound 917)

To a stirred solution of (R)-4-((3-aminopiperidin-1-yl)methyl)-N-(5-(4-morpholino-7H-pyrrolo[2,3-d]pyrimidin-6-yl)pyrazin-2-yl)picolinamide 67 (150 mg, 0.291 mmol) in DMF (3 mL) was added Et₃N (0.12 mL, 0.874 mmol) at 0° C. and stirred for 10 min under N₂ atmosphere. Then, acryloyl chloride 9 (31.7 mg, 0.350 mmol) dissolved in DMF (1 mL) was added dropwise to the reaction mixture at 0° C. and stirred at RT for 15 min. After completion of reaction (TLC and LCMS monitoring), the reaction mixture was poured into ice cold water (50 mL) and extracted with 25% IPA in CHCl₃ (3×50 mL). The combined organic layer was washed with ice cold brine (2×50 mL), dried over Na₂SO₄, filtered and concentrated under reduce pressure. The crude was purified by RP-HPLC [2-98% 0.1% TFA in water/ACN; Column: X Select CSH C18 (19*250) 10μ] to get the title compound Compound 917 (40 mg, 24%).

¹H NMR (400 MHz, DMSO-d₆): δ 12.60 (s, 1H), 9.27 (s, 2H), 8.87 (d, J=4.4 Hz, 1H), 8.41 (s, 1H), 8.25 (s, 1H), 7.85-7.83 (m, 1H), 8.35-8.32 (m, 1H), 7.47 (s, 1H), 6.23-6.01 (m, 2H), 5.66-5.63 (m, 1H), 4.04-4.02 (m, 1H), 3.92-3.91 (m, 4H), 3.78-3.66 (m, 8H), 3.38-3.30 (m, 2H), 2.95-2.93 (m, 1H), 1.90-1.86 (m, 2H), 1.73-1.63 (m, 1H), 1.45-1.39 (m, 1H).

LCMS=[M+H]⁺: 569.39; Purity=99.91%.

### Example 69

### Compound 918

### Preparation of (R)-4-((3-acrylamidopiperidin-1-yl)methyl)-N-(2-(4-morpholino-7H-pyrrolo[2,3-d]pyrimidin-6-yl)pyrimidin-5-yl)picolinamide (Compound 918)

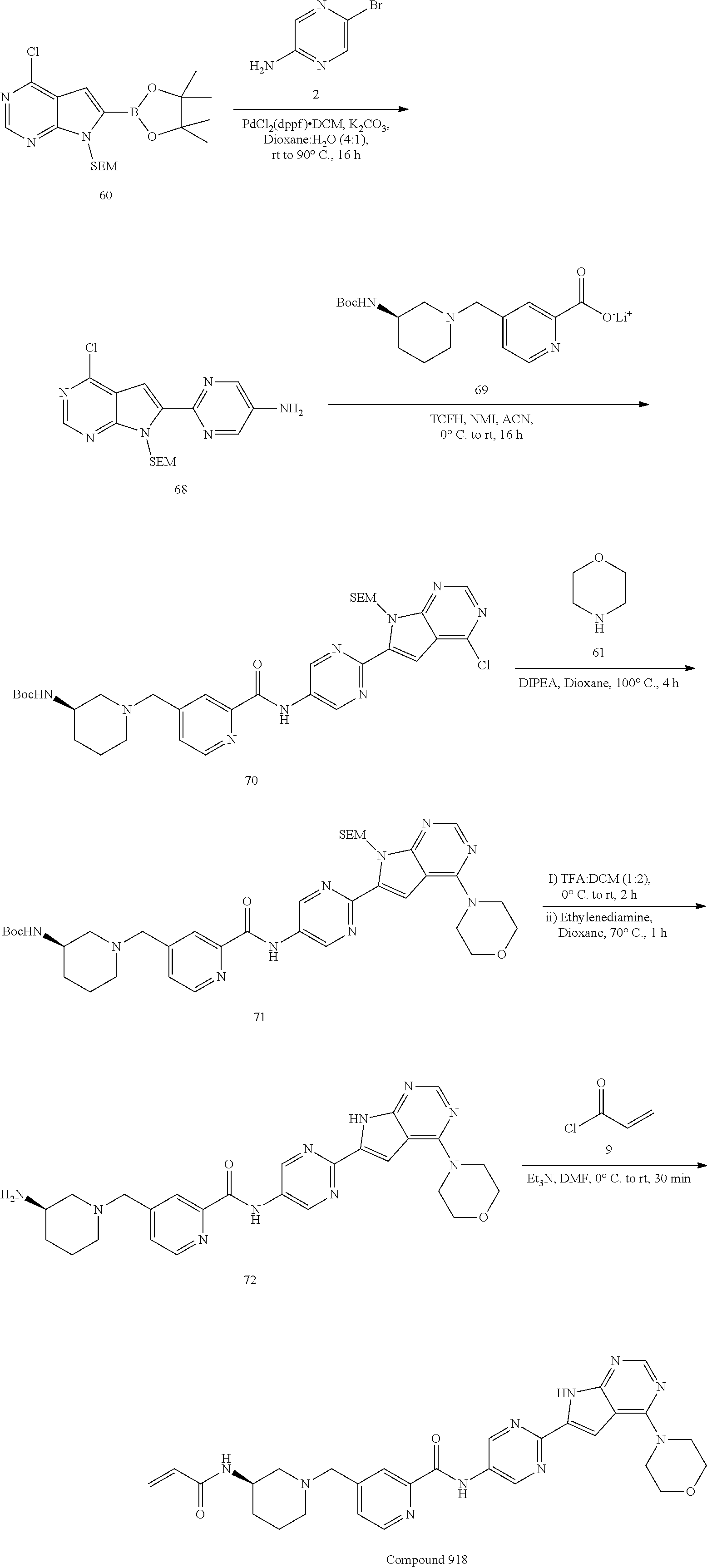

### Synthesis 2-(4-chloro-7-((2-(trimethylsilyl)ethoxy)methyl)-7H-pyrrolo[2,3-d]pyrimidin-6-yl)pyrimidin-5-amine (68)

To a stirred solution of 4-chloro-6-(4,4,5,5-tetramethyl-1,3,2-dioxaborolan-2-yl)-7-((2-(trimethylsilyl)ethoxy)methyl)-7H-pyrrolo[2,3-d]pyrimidine 60 (1 g, 2.44 mmol) in 1,4-dioxane (20 mL) and water (5 mL) were added 2-bromo-5-pyrimidinylamine 2 (467 mg, 2.68 mmol) and K₂CO₃ (1.01 g, 7.32 mmol) at RT. The reaction mixture was degassed with N₂ gas for 10 min, followed by PdCl₂(dppf)·DCM (199 mg, 0.244 mmol) was added to the reaction mixture and stirred at 90° C. for 16 h. After the completion of reaction (TLC monitoring), the reaction mixture was diluted with water (50 mL) and extracted with EtOAc (3×50 mL). The combined organic layer was washed with brine solution (2×50 mL), dried over Na₂SO₄, filtered and evaporated under reduced pressure. The crude was purified by flash chromatography (silica gel 100-200 M, 24 g SNAP) using 65-70% EtOAc in heptane to get the desired product as a pale yellow solid 68 (700 mg, 49%). LCMS=[M+H]⁺: 377.16; Purity=65%.

### Synthesis of tert-butyl (R)-(1-((2-((2-(4-chloro-7-((2-(trimethylsilyl)ethoxy)methyl)-7H-pyrrolo[2,3-d]pyrimidin-6-yl)pyrimidin-5-yl)carbamoyl)pyridin-4-yl)methyl)piperidin-3-yl)carbamate (70)

To a stirred solution of 2-(4-chloro-7-((2-(trimethylsilyl)ethoxy)methyl)-7H-pyrrolo[2,3-d]pyrimidin-6-yl)pyrimidin-5-amine 68 (700 mg, 1.85 mmol) in ACN (10 mL) were added and lithium (R)-4-((3-((tert-butoxycarbonyl)amino)piperidin-1-yl)methyl)picolinate 69 (951 mg, 2.78 mmol), 1-methylimidazole (0.4 mL, 4.84 mmol) and TCFH (1.04 g, 3.71 mmol) to the reaction mixture at RT was added under N₂ atmosphere and stirred for 16 h. After completion of reaction (TLC monitoring and LCMS), the reaction was diluted with water (50 mL) and extracted with EtOAc (3×50 mL). The combined organic layer was washed with brine (2×20 mL), dried over Na₂SO₄, filtered and concentrated under reduced pressure to get desired product as a brown solid 70 (1.10 g; crude) which was used in next step without further purification.

LCMS=[M+H]⁺: 694.30

### Synthesis of tert-butyl (R)-(1-((2-((2-(4-morpholino-7-((2-(trimethylsilyl)ethoxy)methyl)-7H-pyrrolo[2,3-d]pyrimidin-6-yl)pyrimidin-5-yl)carbamoyl)pyridin-4-yl)methyl)piperidin-3-yl)carbamate (71)

To a stirred solution of tert-butyl (R)-(1-((2-((2-(4-chloro-7-((2-(trimethylsilyl)ethoxy)methyl)-7H-pyrrolo[2,3-d]pyrimidin-6-yl)pyrimidin-5-yl)carbamoyl)pyridin-4-yl)methyl)piperidin-3-yl)carbamate 70 (1.1 g, 1.58 mmol) in DMSO (10 mL) were added DIPEA (1.4 mL, 7.92 mmol) and morpholine 61 (0.4 mL, 4.75 mmol) sequentially at RT and stirred at 120° C. for 4 h. After completion of reaction (TLC monitoring), the reaction mixture was cooled to RT, poured into ice cold-water (50 mL) and extracted with EtOAc (3×50 mL). The combined organic layer was washed with ice cold brine (2×30 mL), dried over Na₂SO₄, filtered and evaporated under reduced pressure. The crude was purified by flash chromatography (silica gel 100-200 M, 40 g SNAP) using 20-30% EtOAc in heptane to get the desired product as a yellow solid 71 (1 g). LCMS=[M+H]⁺: 745.39

### Synthesis of (R)-4-((3-aminopiperidin-1-yl)methyl)-N-(2-(4-morpholino-7H-pyrrolo[2,3-d]pyrimidin-6-yl)pyrimidin-5-yl)picolinamide (72)

To a stirred solution of tert-butyl (R)-(1-((2-((2-(4-morpholino-7-((2-(trimethylsilyl)ethoxy)methyl)-7H-pyrrolo[2,3-d]pyrimidin-6-yl)pyrimidin-5-yl)carbamoyl)pyridin-4-yl)methyl)piperidin-3-yl)carbamate 71 (1 g, 1.34 mmol) in DCM (10 mL) was added TFA (5 mL) dropwise at 0° C. and stirred at RT for 2 h. After consumed of starting material (monitoring by TLC), the reaction mixture was concentrated under reduced pressure. The crude was dissolved in 1,4-dioxane (10 mL) and 1,2-ethanediamine (0.18 mL, 2.68 mmol) was added at RT and stirred at 70° C. for 1 h. After completion of reaction (LCMS monitoring), the reaction mixture was diluted with 25% IPA in CHCl₃ (50 mL) and washed with ice cold water (2×30 mL). The organic layer was dried over Na₂SO₄, filtered and concentrated under reduced pressure to get the desired compound as a yellow solid 72 (450 mg; crude) which was used in next step directly without further purification. LCMS=[M+H]′: 515.26; Purity=54%

### Synthesis of N-[2-(4-morpholino-1H-1,5,7-triazainden-2-yl)-5-pyrimidinyl]-4-{[(R)-3-(vinylcarbonylamino)-1-piperidyl]methyl}-2-pyridinecarboxamide (Compound 918)

To a stirred solution of (R)-4-((3-aminopiperidin-1-yl)methyl)-N-(2-(4-morpholino-7H-pyrrolo[2,3-d]pyrimidin-6-yl)pyrimidin-5-yl)picolinamide 72 (430 mg, 0.836 mmol) in DMF (5 mL) was added Et₃N (0.35 mL, 2.51 mmol) at 0° C. under N₂ atmosphere and stirred for 10 min. Then, acryloyl chloride 9 (83.2 mg, 0.919 mmol) dissolved in DMF (2 mL) was added to the reaction mixture at 0° C. and stirred at RT for 30 min. After completion of reaction (TLC and LCMS monitoring), the reaction mixture was quenched with ice cold water (50 mL) and extracted with 25% IPA in CHCl₃ (3×50 mL). The combined organic was washed with brine (2× 20 mL) and dried over Na₂SO₄, filtered and concentrated under reduced pressure. The crude was purified by RP-HPLC to get the title compound Compound 918 (23 mg, 5%).

¹H NMR (400 MHz, DMSO-d₆): δ 12.64 (s, 1H), 11.34 (s, 1H), 9.38 (s, 2H), 8.90 (d, J=4.4 Hz, 1H), 8.40 (s, 1H), 8.32-8.27 (m, 2H), 7.82 (d, J=3.6 Hz, 1H), 7.50 (s, 1H), 6.20-6.19 (m, 1H), 6.16 (dd, J=16.8 Hz, 2.4 Hz, 1H), 5.66 (dd, J=10 Hz, 2 Hz, 1H), 4.54 (s, 2H), 4.05-4.36 (m, 11H), 2.95-2.91 (m, 1H), 2.75-2.71 (m, 1H), 1.94-1.86 (m, 2H), 1.75-1.68 (m, 1H), 1.45-1.33 (m, 1H).

LCMS=[M+H]⁺: 569.25; Purity=98.59%.

### Example 70

### Compound 919

### Preparation of 1-(4-(1-((4-(4-(3-oxa-8-azabicyclo[3.2.1]octan-8-yl)-7H-pyrrolo[2,3-d]pyrimidin-6-yl)phenyl)amino)-2,2,2-trifluoroethyl)piperidin-1-yl)-2-((3,3-difluoroazetidin-1-yl)methyl)prop-2-en-1-one (Compound 919)

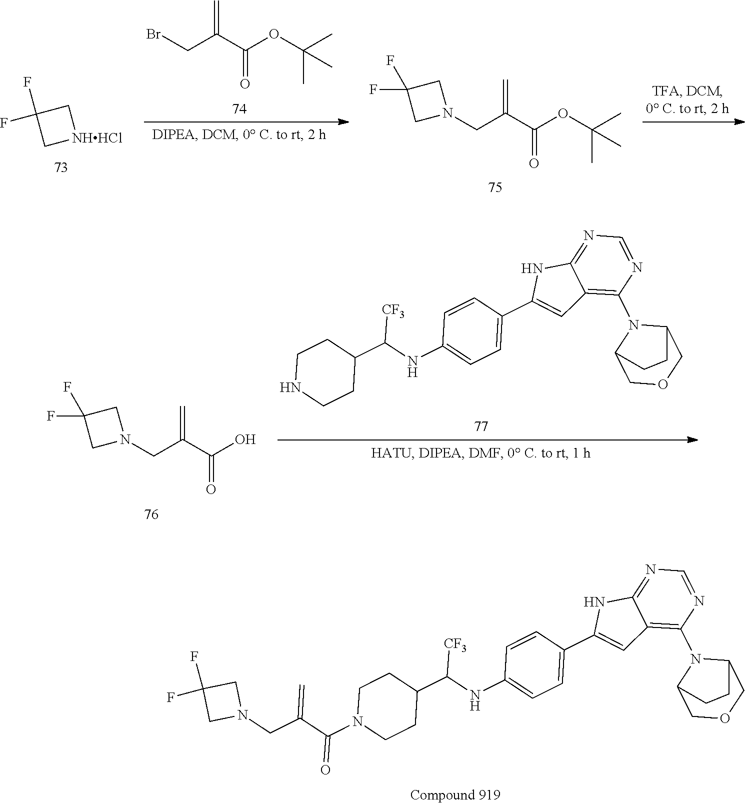

### Synthesis of tert-butyl 2-((3,3-difluoroazetidin-1-yl)methyl)acrylate (75)

To a stirred solution of 3,3-difluoroazetidine hydrochloride 73 (200 mg, 2.15 mmol) in DCM (10 mL) were added and tert-butyl 2-(bromomethyl)acrylate 74 (570 mg, 2.58 mmol) and DIPEA (1.12 mL, 6.45 mmol) at 0° C. under N₂ atmosphere and stirred at RT for 2 h. After completion of reaction (monitoring by TLC), the reaction mass was poured into water (50 mL) and extracted with DCM (3×50 mL). The combined organic layer was dried over Na₂SO₄, filtered and concentrated under reduced pressure to get the desired compound as oily liquid 75 (250 mg, 50%) which was used in next step without purification.

LCMS=[M+H]⁺: 234.36; Purity=85%.

### Synthesis of 2-((3,3-difluoroazetidin-1-yl)methyl)acrylic acid (76)

To a stirred solution of tert-butyl 2-((3,3-difluoroazetidin-1-yl)methyl)acrylate 75 (250 mg, 1.07 mmol) in DCM (5 mL) was added TFA (1.64 mL, 21.4 mmol) dropwise at 0° C. and stirred at RT for 2 h. After completion of reaction (TLC monitoring), the reaction mixture was concentrated under reduced pressure. The crude was triturated with diethyl ether to get the desired product as a yellow solid 76 (100 mg; as TFA salt) which was used in next step directly without further purification.

LCMS=[M+H]⁺: 178.27; Purity=59%

### Synthesis of 1-(4-(1-((4-(4-(3-oxa-8-azabicyclo[3.2.1]octan-8-yl)-7H-pyrrolo[2,3-d]pyrimidin-6-yl)phenyl)amino)-2,2,2-trifluoroethyl)piperidin-1-yl)-2-((3,3-difluoroazetidin-1-yl)methyl)prop-2-en-1-one (Compound 919)

To a stirred solution of 2-((3,3-difluoroazetidin-1-yl)methyl)acrylic acid 76 (87.4 mg, 0.493 mmol) and 4-(4-(3-oxa-8-azabicyclo[3.2.1]octan-8-yl)-7H-pyrrolo[2,3-d]pyrimidin-6-yl)-N-(2,2,2-trifluoro-1-(piperidin-4-yl)ethyl)aniline 77 (200 mg, 0.411 mmol) in DMF (5 mL) were added DIPEA (0.22 mL, 1.23 mmol) and HATU (313 mg, 0.822 mmol) sequentially at 0° C. under N₂ atmosphere and stirred at RT for 1 h. After completion of reaction (TLC and LCMS monitoring), the reaction mixture was diluted with water (20 mL) and extracted with 25% IPA in CHCl₃ (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get the title compound Compound 919 (41 mg, 15%).

¹H NMR (400 MHz, DMSO-d₆): δ 11.94 (brs, 1H), 8.12 (s, 1H), 7.65 (d, J=8.4 Hz, 2H), 6.86-6.84 (m, 3H), 6.09 (d, J=10 Hz, 1H), 5.32 (s, 1H), 5.11-5.10 (m, 1H), 4.87 (brs, 2H), 4.39-4.32 (m, 2H), 3.90-3.82 (m, 1H), 3.69 (d, J=10.8 Hz, 2H), 3.63-3.54 (m, 6H), 3.32-3.28 (m, 2H), 3.05-3.01 (m, 1H), 2.65-2.62 (m, 1H), 2.21-1.94 (m, 5H), 1.78-1.68 (m, 2H), 1.40-1.28 (m, 2H).

LCMS=[M+H]⁺: 646.22; Purity=99.13%.

### Example 71

### Compound 920

### Preparation of (R)—N-(1-(5-((4-(4-morpholino-7H-pyrrolo[2,3-d]pyrimidin-6-yl)phenyl)amino)pyrimidin-2-yl)pyrrolidin-3-yl)acrylamide (Compound 920)

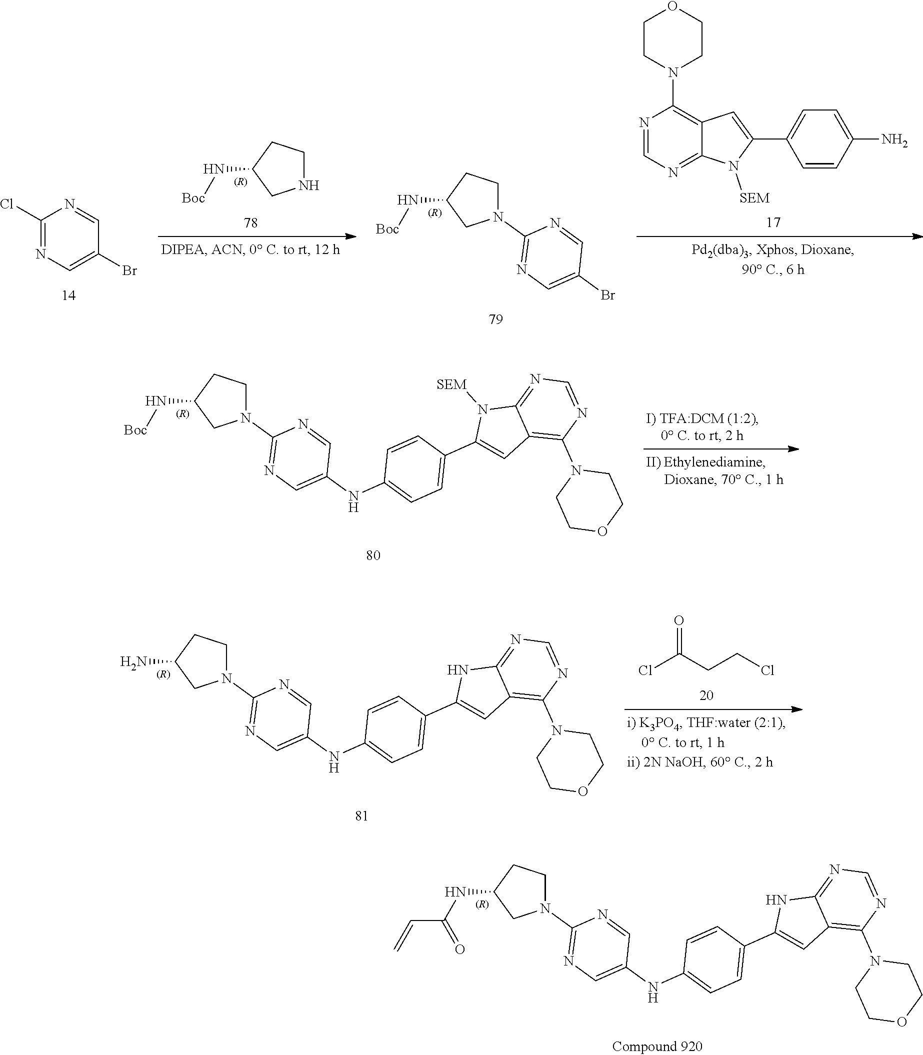

### Synthesis of tert-butyl (R)-(1-(5-bromopyrimidin-2-yl)pyrrolidin-3-yl)carbamate (79)

To a stirred solution of 5-bromo-2-chloropyrimidine 14 (800 mg, 4.14 mmol) in ACN (15 mL) were added tert-butyl (R)-pyrrolidin-3-ylcarbamate 78 (770 mg, 4.14 mmol) and DIPEA (2.15 mL, 12.4 mmol) at 0° C. and stirred at RT for 12 h. After completion of reaction (monitoring by TLC), the reaction mixture was concentrated under reduced pressure. The crude was poured into ice cold water (50 mL) and extracted with EtOAc (3×50 mL). The combined organic layer was washed with ice cold brine (2×50 mL), dried over Na2SO₄, filtered and evaporated under reduced pressure to get the titled compound as an off white solid 79 (1.2 g, 70%).

¹H NMR (400 MHz, DMSO-d₆): δ 8.43 (s, 2H), 7.21 (s, 1H), 4.10-4.09 (m, 1H), 3.69-3.52 (m, 3H), 3.52-3.42 (m, 1H), 2.13-2.08 (m, 1H), 1.87-1.84 (m, 1H), 1.38 (s, 9H).

### Synthesis of tert-butyl (R)-(1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)pyrrolidin-3-yl)carbamate (80)

To a stirred solution of tert-butyl (R)-(1-(5-bromopyrimidin-2-yl)pyrrolidin-3-yl)carbamate 79 (1.2 g, 3.5 mmol) in 1,4-dioxane (20 mL) were added 4-(4-morpholino-7-((2-(trimethylsilyl)ethoxy)methyl)-7H-pyrrolo[2,3-d]pyrimidin-6-yl)aniline 17 (1.49 g, 3.5 mmol) and NaO Bu (672 mg, 6.99 mmol) at RT. The reaction mixture was degassed with N₂ gas for 10 min, followed by XPhos (333 mg, 0.699 mmol) and Pd₂(dba)₃ (283 mg, 0.350 mmol) were added to the reaction mixture and stirred at 90° C. for 6 h. After completion of reaction (monitoring by TLC), the reaction mixture was poured into water (100 mL) and extracted with EtOAc (2×100 mL). The combined organic layer was washed with brine solution (2×50 mL), dried over Na₂SO₄, filtered and concentrated under reduced pressure. The crude was purified by flash chromatography (silica gel 100-200, 24 g SNAP) using 35-40% EtOAc in heptane to get the title compound as a pale yellow solid 80 (1 g, 29%).

LCMS=[M+H]⁺: 688.48; Purity=70%

### Synthesis of (R)-2-(3-aminopyrrolidin-1-yl)-N-(4-(4-morpholino-7H-pyrrolo[2,3-d]pyrimidin-6-yl)phenyl)pyrimidin-5-amine (81)

To a stirred solution of tert-butyl (R)-(1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)pyrrolidin-3-yl)carbamate 80 (1 g, 1.45 mmol) in DCM (10 mL) was added TFA (5 mL) dropwise at 0° C. and stirred at RT for 2 h. After consumed of starting material (monitoring by TLC), the reaction mixture was concentrated under reduced pressure. The crude was triturated with diethyl ether, and dried under reduced pressure. The crude was dissolved in 1,4-dioxane (10 mL) and 1,2-ethanediamine (0.2 mL, 2.91 mmol) was added to the reaction mixture at RT and stirred at 70° C. for 1 h. After completion of reaction (LCMS monitoring), the reaction mixture was diluted water (50 mL) and extracted with 25% IPA in CHCl₃ (3×50 mL). The organic was dried over Na₂SO₄, filtered and concentrated to get the title compound as a pale yellow solid 81 (610 mg; crude) which was used in next step directly without further purification.

LCMS=[M+H]⁺: 458.24; Purity=82%.

### Synthesis of (R)—N-(1-(5-((4-(4-morpholino-7H-pyrrolo[2,3-d]pyrimidin-6-yl)phenyl)amino)pyrimidin-2-yl)pyrrolidin-3-yl)acrylamide (Compound 920)

To a stirred solution of (R)-2-(3-aminopyrrolidin-1-yl)-N-(4-(4-morpholino-7H-pyrrolo[2,3-d]pyrimidin-6-yl)phenyl)pyrimidin-5-amine 81 (610 mg, 1.33 mmol) in THF (10 mL) was added K₃PO₄ (566 mg, 2.67 mmol) dissolved in water (5 mL) at 0° C. Then, 3-chloropropanoyl chloride 20 (203 mg, 1.6 mmol) was added dropwise to the reaction mixture at 0° C. and stirred at RT for 1 h. After consumed of starting material (monitoring by TLC), 2N NaOH (8 mL, 16 mmol) was added dropwise to the reaction mixture at 0° C. and stirred at 60° C. for 2 h. After completion of reaction (LCMS monitoring), the reaction mixture was diluted water (50 mL) and extracted with 2-MeTHF (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get the title Compound 920 (215 mg, 32%).

¹H NMR (400 MHz, DMSO-d₆): $12.05 (s, 1H), 8.39 (d, J=6 Hz, 1H), 8.29 (s, 2H), 8.13 (s, 1H), 7.84 (s, 1H), 7.68 (d, J=8.8 Hz, 2H), 6.90 (s, 1H), 6.76 (d, J=8.4 Hz, 2H), 6.27-6.20 (m, 1H), 6.11 (dd, J=17.2 Hz, 2 Hz, 1H), 5.60 (dd, J=10 Hz, 2.2 Hz, 1H), 4.43-4.40 (m, 1H), 3.86-3.83 (m, 4H), 3.74-3.70 (m, 5H), 3.61-3.58 (m, 2H), 3.44-3.32 (m, 1H), 2.22-2.16 (m, 1H), 1.94-1.90 (m, 1H).

LCMS=[M+H]⁺: 512.26; Purity=99.46%.

### Example 72

### Compound 921

### Preparation of N-((3R)-1-(5-((4-(4-(3-oxa-8-azabicyclo[3.2.1]octan-8-yl)-7H-pyrrolo[2,3-d]pyrimidin-6-yl)phenyl)amino)pyrimidin-2-yl)piperidin-3-yl)acrylamide (Compound 921)

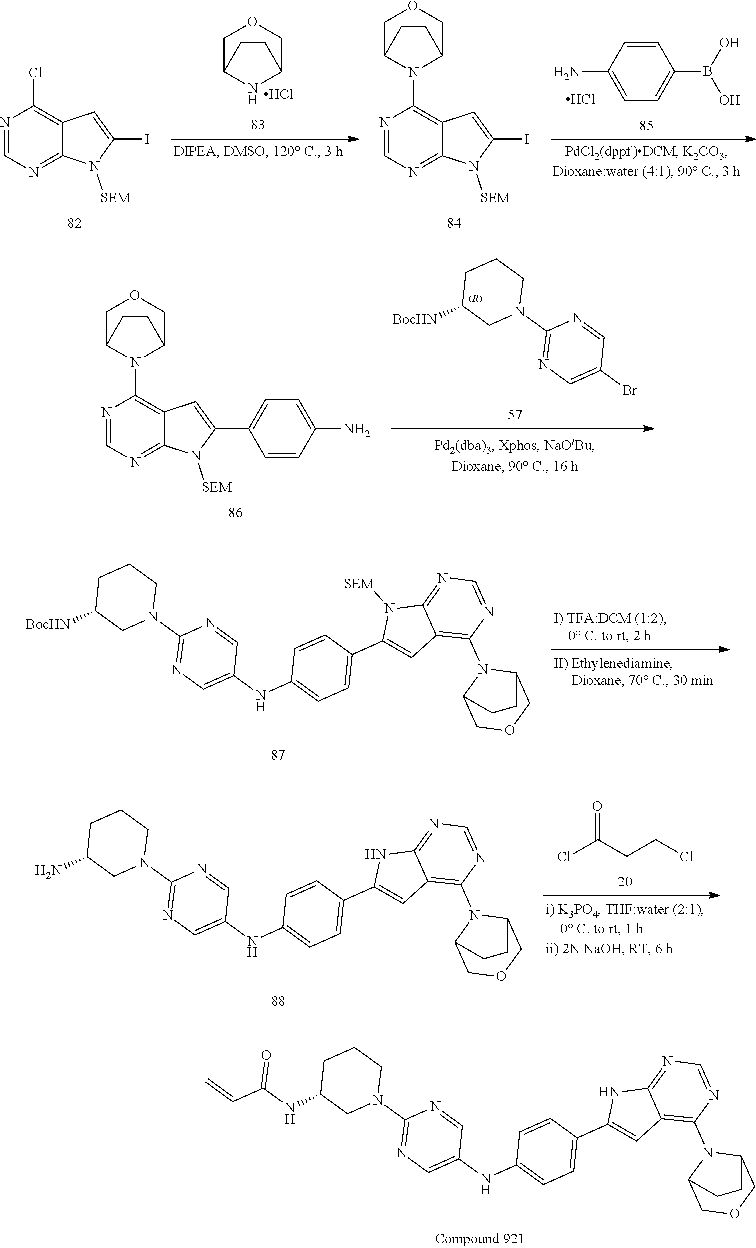

### Synthesis of (1R,5S)-8-(6-iodo-7-((2-(trimethylsilyl)ethoxy)methyl)-7H-pyrrolo[2,3-d]pyrimidin-4-yl)-3-oxa-8-azabicyclo[3.2.1]octane (84)

To a stirred solution of 4-chloro-6-iodo-7-((2-(trimethylsilyl)ethoxy)methyl)-7H-pyrrolo[2,3-d]pyrimidine 82 (16 g, 39.1 mmol) in DMSO (100 mL) were added DIPEA (20.4 mL, 117 mmol) and 3-oxa-8-azabicyclo[3.2.1]octane hydrogen chloride 83 (8.76 g, 58.6 mmol) sequentially at RT and stirred at 120° C. for 3 h. After completion of reaction (TLC monitoring), the reaction mixture was cooled to RT and poured into ice cold water (200 mL) and extracted with EtOAc (2×100 mL). The combined organic layer was washed with ice cold brine (2×100 mL), dried over Na₂SO₄, filtered and evaporated under reduced pressure. The crude was purified by flash chromatography (silica gel 100-200 M, 120 g SNAP) using 30-40% ethyl acetate in heptane to get the title compound as sticky brown liquid 84 (13.7 g, 71%).

LCMS=[M+H]⁺: 487.25; Purity=79%

### Synthesis of 4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)aniline (86)

To a stirred solution of (1R,5S)-8-(6-iodo-7-((2-(trimethylsilyl)ethoxy)methyl)-7H-pyrrolo[2,3-d]pyrimidin-4-yl)-3-oxa-8-azabicyclo[3.2.1]octane 84 (1.5 g, 3.08 mmol) in 1,4-dioxane (20 mL) and water (5 mL) were added (4-aminophenyl) boronic acid hydrochloride 85 (1.07 g, 6.17 mmol) and K₂CO₃ (1.28 g, 9.25 mmol) at RT. The reaction mixture was degassed with N₂ gas for 10 min, followed by PdCl₂(dppf)·DCM (252 mg, 0.308 mmol) was added to the reaction mixture and stirred at 90° C. for 3 h. After the completion of reaction (TLC monitoring), the reaction mixture was diluted with water (100 mL) and extracted with EtOAc (3×100 mL). The combined organic layer was washed with brine solution (2×50 mL), dried over Na₂SO₄, filtered and evaporated under reduced pressure. The crude was purified by flash chromatography (silica gel 100-200 M, 40 g SNAP) using 30-35% EtOAc in heptane to get the desired product as a brown sticky solid 86 (1.2 g, 57%). LCMS=[M+H]⁺: 452.42; Purity=93%

### Synthesis of tert-butyl ((3R)-1-(5-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)pyrimidin-2-yl)piperidin-3-yl)carbamate (87)

To a stirred solution of tert-butyl (R)-(1-(5-bromopyrimidin-2-yl)piperidin-3-yl)carbamate 57 (380 mg, 1.06 mmol) 1,4-dioxane (10 mL) were added 4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)aniline 86 (480 mg, 1.06 mmol) and NaO′Bu (204 mg, 2.13 mmol) at RT. The reaction mixture was purged with N₂ gas for 10 min, followed by XPhos (101 mg, 0.213 mmol) and Pd₂(dba)₃ (86.1 mg, 0.106 mmol) were added to the reaction mixture and stirred at 90° C. for 16 h. After completion of reaction (TLC monitoring), the reaction was diluted with water (50 mL) and extracted with EtOAc (3×50 mL). The combined organic layer was washed with brine solution (2×50 mL), dried over Na₂SO₄, filtered and concentrated under reduced pressure. The crude was purified by flash chromatography (silica gel 100-200 M, 12 g SNAP) using 3-5% MeOH in DCM to afford the title compound as a light brown solid 87 (400 mg, 29%).

LCMS=[M+H]⁺: 728.45; Purity=65%

### N-(4-(4-(3-oxa-8-azabicyclo[3.2.1]octan-8-yl)-7H-pyrrolo[2,3-d]pyrimidin-6-yl)phenyl)-2-((R)-3-aminopiperidin-1-yl)pyrimidin-5-amine (88)

To a stirred solution of tert-butyl ((3R)-1-(5-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)pyrimidin-2-yl)piperidin-3 -yl)carbamate 87 (380 mg, 0.522 mmol) in DCM (10 mL) was added TFA (5 mL) dropwise at 0° C. and stirred at RT for 2 h. After consumed of starting material (TLC monitoring), the reaction mixture was concentrated under reduced pressure. The crude was triturated with diethyl ether and dried to obtain the solid. The crude was dissolved in 1,4-dioxane (5 mL) and 1,2-ethanediamine (69.7 μL, 1.04 mmol) was added to the reaction mixture and stirred at 70° C. for 30 min. After completion of reaction (monitoring by LCMS), the reaction mixture was evaporated under reduced pressure. The crude was dissolved in 25% IPA in CHCl₃ (50 mL) and washed with ice cold water (2×50 mL). The organic was dried over Na₂SO₄, filtered and concentrated under reduced pressure to get the title compound as a yellow solid 88 (215 mg; crude) which was used as such for the next step.

LCMS=[M+H]⁺: 498.31; Purity=60%

### Synthesis of N-((3R)-1-(5-((4-(4-(3-oxa-8-azabicyclo[3.2.1]octan-8-yl)-7H-pyrrolo[2,3-d]pyrimidin-6-yl)phenyl)amino)pyrimidin-2-yl)piperidin-3-yl)acrylamide (Compound 921)

To a stirred solution of N-(4-(4-(3-oxa-8-azabicyclo[3.2.1]octan-8-yl)-7H-pyrrolo[2,3-d]pyrimidin-6-yl)phenyl)-2-((R)-3-aminopiperidin-1-yl)pyrimidin-5-amine 88 (200 mg, 0.402 mmol) in THF (4 mL) was added K₃PO₄ (171 mg, 0.804 mmol) dissolved in water (2 mL) at 0° C. Then, 3-chloropropanoyl chloride 20 (62 mg, 1.6 mmol) dissolved in THF (1 mL) was added dropwise to the reaction mixture at 0° C. and stirred at RT for 1 h. After consumed of starting material (monitoring by TLC), 2N NaOH (2.5 mL, 4.82 mmol) was added dropwise to the reaction mixture at 0° C. and stirred at RT for 6 h. After completion of reaction (LCMS monitoring), the reaction mixture was diluted water (50 mL) and extracted with 2-MeTHF (3×50 mL). The combined organic layer was dried over Na₂SO₄, filtered and concentrated under reduced pressure. The crude was purified by RP-HPLC [2-98% 5 mM Ammonium Bicarbonate in Water/ACN; Column: X Bridge C8 (19*250) 5μ] to get the title Compound 921 (24 mg, 11%).

¹H NMR (400 MHz, DMSO-d₆): $12.00 (s, 1H), 8.29 (s, 2H), 8.13 (s, 1H), 8.10 (d, J=8 Hz, 1H), 7.89 (s, 1H), 7.71 (d, J=8.8 Hz, 2H), 6.87 (d, J=1.2 Hz, 1H), 6.80 (d, J=8.8 Hz, 2H), 6.28-6.21 (m, 1H), 6.10 (dd, J=17.2 Hz, 2.4 Hz, 1H), 5.59 (dd, J=10 Hz, 2 Hz, 1H), 4.87 (brs, 2H), 4.44-4.40 (m, 1H), 4.30-4.26 (m, 1H), 3.76-3.74 (m, 1H), 3.70-3.68 (m, 2H), 3.62-3.59 (m, 2H), 3.13-3.05 (m, 1H), 2.96-2.90 (m, 1H), 2.02-1.93 (m, 3H), 1.90-1.86 (m, 2H), 1.79-1.75 (m, 1H), 1.51-1.46 (m, 2H).

LCMS=[M+H]⁺: 552.26; Purity=96.24%

### Example 73

### Compound 922

### Preparation of (R)—N-(1-(5-((2-methyl-4-(4-morpholino-7H-pyrrolo[2,3-d]pyrimidin-6-yl)phenyl)amino)pyrimidin-2-yl)piperidin-3-yl)acrylamide (Compound 922)

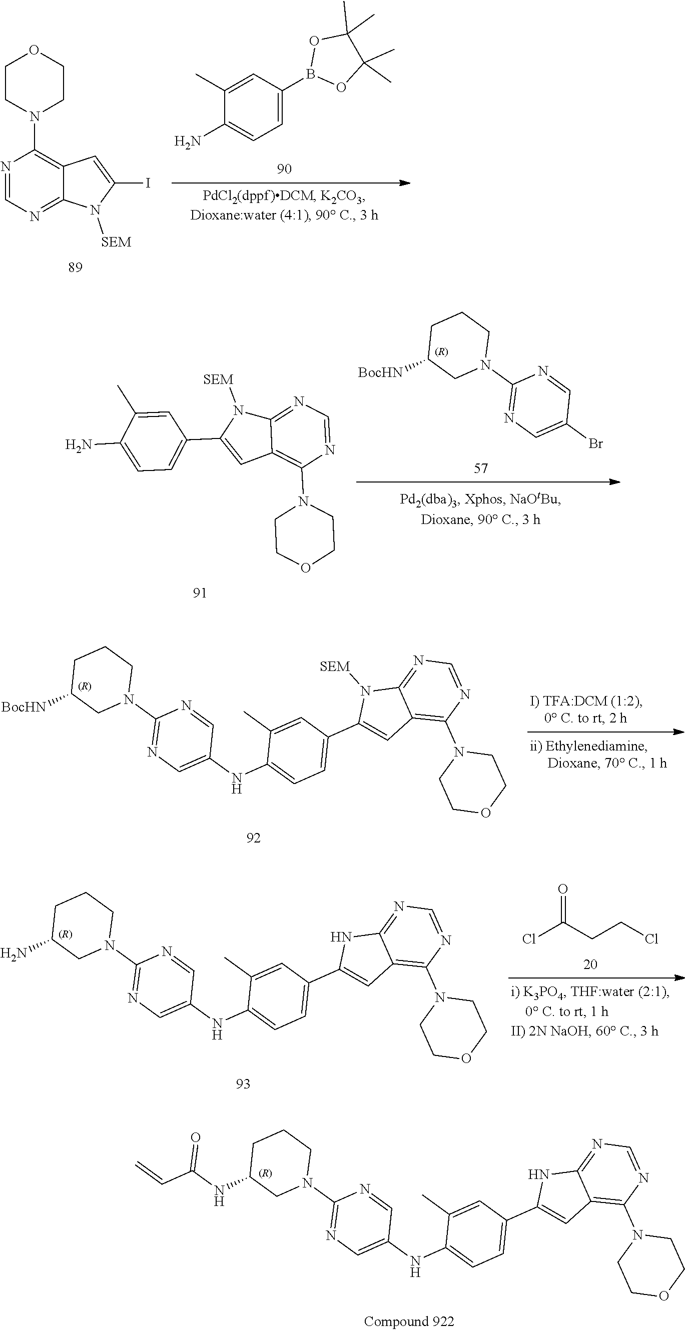

### Synthesis of 2-methyl-4-(4-morpholino-7-((2-(trimethylsilyl)ethoxy)methyl)-7H-pyrrolo[2,3-d]pyrimidin-6-yl)aniline (91)

To a stirred solution of 4-(6-iodo-7-((2-(trimethylsilyl)ethoxy)methyl)-7H-pyrrolo[2,3-d]pyrimidin-4-yl)morpholine 89 (2 g, 4.34 mmol) in 1,4-dioxane (20 mL) and water (5 mL) were added and 4-(4,4,5,5-tetramethyl-1,3,2-dioxaborolan-2-yl)-2-tolylamine 90 (1.01 g, 4.34 g) and K₂CO₃ (1.8 g, 13 mmol) at RT. The reaction mixture was degassed with N₂ gas for 15 min. Then, PdCl₂(dppf)·DCM (354 mg, 0.434 mmol) was added to the reaction mixture and stirred at 90° C. for 3 h. After the completion of reaction (TLC monitoring), the reaction mixture was diluted with water (50 mL) and extracted with EtOAc (2×100 mL). The combined organic layer was washed with brine solution (2×50 mL), dried over Na₂SO₄, filtered and evaporated under reduced pressure. The crude was purified by flash chromatography (silica gel 100-200 M, 40 g SNAP) using 20-30% EtOAc in heptane to get the desired product as a yellow solid 91 (1 g, 52%).

LCMS=[M+H]⁺: 440.24; Purity=89%

### Synthesis of tert-butyl (R)-(1-(5-((2-methyl-4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl)carbamate (92)

To a stirred solution of 2-methyl-4-(4-morpholino-7-((2-(trimethylsilyl)ethoxy)methyl)-7H-pyrrolo[2,3-d]pyrimidin-6-yl)aniline 91 (1 g, 2.27 mmol) in 1,4-dioxane (15 mL) were added and tert-butyl (R)-(1-(5-bromopyrimidin-2-yl)piperidin-3-yl)carbamate 57 (975 mg, 2.73 mmol) and NaO′Bu (328 mg, 3.41 mmol) to the reaction mixture at RT. The resulting reaction mixture was degassed with N₂ gas for 10 min, followed by XPhos (217 mg, 0.455 mmol) and Pd₂(dba)₃ (184 mg, 0.227 mmol) were added to the reaction mixture and stirred at 90° C. for 3 h. After completion of reaction (TLC monitoring), the reaction mass was poured into ice cold water (50 mL) and extracted with EtOAc (3×50 mL). The combined organic layer was washed with brine solution (2×50 mL), dried over Na₂SO₄, filtered and concentrated under reduced pressure. The crude was purified by column chromatography using 30-35% EtOAc in heptane to get the title compound as a pale yellow solid 92 (800 mg, 37%).

LCMS=[M+H]⁺: 716.52; Purity=77%.

### Synthesis of (R)-2-(3-aminopiperidin-1-yl)-N-(2-methyl-4-(4-morpholino-7H-pyrrolo[2,3-d]pyrimidin-6-yl)phenyl)pyrimidin-5-amine (93)

To a stirred solution of tert-butyl (R)-(1-(5-((2-methyl-4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl) carbamate 92 (800 mg, 1.12 mmol) in DCM (8 mL) was added TFA (4 mL) at 0° C. and stirred at RT for 2 h. After consumed of starting material (TLC and LCMS monitoring), the reaction mixture was concentrated under reduced pressure. The crude was dissolved in 1,4-dioxane (8 mL) and 1,2-ethanediamine (0.25 mL, 3.35 mmol) was added to the reaction mixture and stirred at 70° C. for 1 h. After completion of reaction (LCMS monitoring), the reaction mixture was diluted with water (50 mL) and extracted with 25% IPA in CHCl₃ (3×50 mL). The combined organic layer was dried over Na₂SO₄, filtered and concentrated under reduced pressure to get the desired compound as a yellow solid 93 (500 mg; crude) which was in next step directly without further purification.

LCMS=[M+H]⁺: 486.51; Purity=84%

### Synthesis of (R)—N-(1-(5-((2-methyl-4-(4-morpholino-7H-pyrrolo[2,3-d]pyrimidin-6-yl)phenyl)amino)pyrimidin-2-yl)piperidin-3-yl)acrylamide (Compound 922)

To a stirred solution of (R)-2-(3-aminopiperidin-1-yl)-N-(2-methyl-4-(4-morpholino-7H-pyrrolo[2,3-d]pyrimidin-6-yl)phenyl)pyrimidin-5-amine 93 (500 mg, 1.03 mmol) in THF (5 mL) was added K₃PO₄ (1.09 g, 5.15 mmol) dissolved in water (2.5 mL) at 0° C. Then, 3-chloropropionyl chloride 20 (157 mg, 1.24 mmol) was added dropwise to the reaction mixture at 0° C. and stirred at RT for 1 h. After consumed of starting material (monitoring by TLC), 2N NaOH (6.2 mL, 12.4 mmol) was added dropwise to the reaction mixture at 0° C. and stirred at 60° C. for 3 h. After completion of reaction (LCMS monitoring), the reaction mixture was diluted with water (50 mL) and extracted with 2-MeTHF (3×50 mL). The combined organic layer was dried over Na₂SO₄, filtered and concentrated under reduced pressure. The crude was purified by RP-HPLC [2-98% 0.1% TFA in Water/ACN; Column: X Select Phenyl Hexyl (19*250) 5μ] to get the titled compound Compound 922 (290 mg, 52%).

¹H NMR (400 MHz, DMSO-d₆): δ 12.73 (brs, 1H), 8.30 (s, 1H), 8.23 (s, 2H), 8.12 (d, J=8 Hz, 1H), 7.71 (s, 1H), 7.57-7.54 (m, 1H), 7.15 (s, 2H), 6.69 (d, J=8.4 Hz, 1H), 6.28-6.21 (m, 1H), 6.10 (dd, J=16.8 Hz, 2.4 Hz, 1H), 5.59 (dd, J=10 Hz, 2.4 Hz, 1H), 4.48-4.44 (m, 1H), 4.33-4.29 (m, 1H), 3.94-3.92 (m, 4H), 3.81-3.76 (m, 5H), 3.09-3.04 (m, 1H), 2.94-2.88 (m, 1H), 2.29 (s, 3H), 1.95-1.90 (m, 1H), 1.82-1.78 (m, 1H), 1.51-1.47 (m, 2H).

LCMS=[M+H]⁺: 540.29; Purity=99.72%.

### Example 74

### Compound 923

### Preparation of N-((3R)-1-((2-(5-(4-(4-(3-oxa-8-azabicyclo[3.2.1]octan-8-yl)-7H-pyrrolo[2,3-d]pyrimidin-6-yl)phenyl)-1H-imidazol-2-yl)pyridin-4-yl)methyl)piperidin-3-yl)acrylamide (Compound 923)

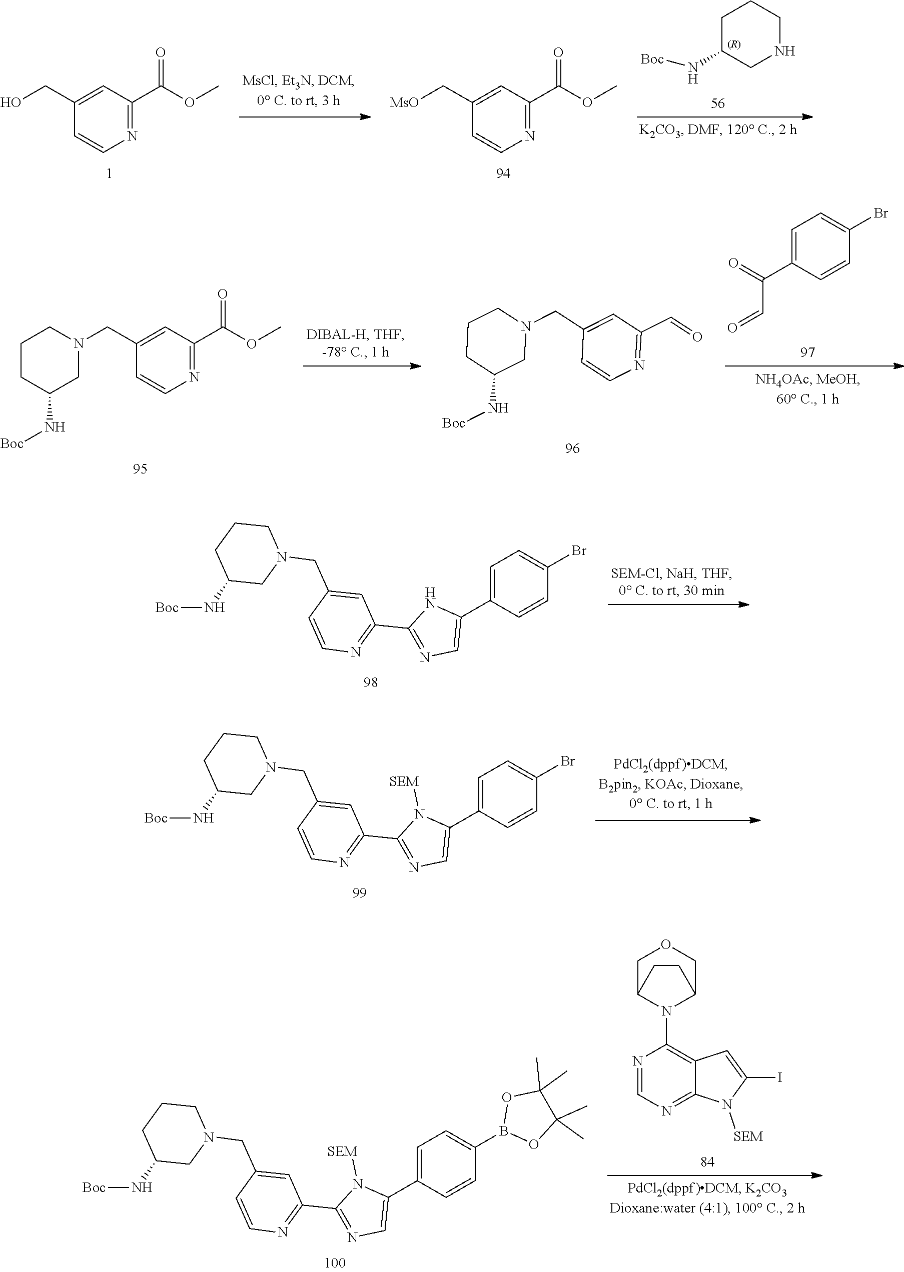

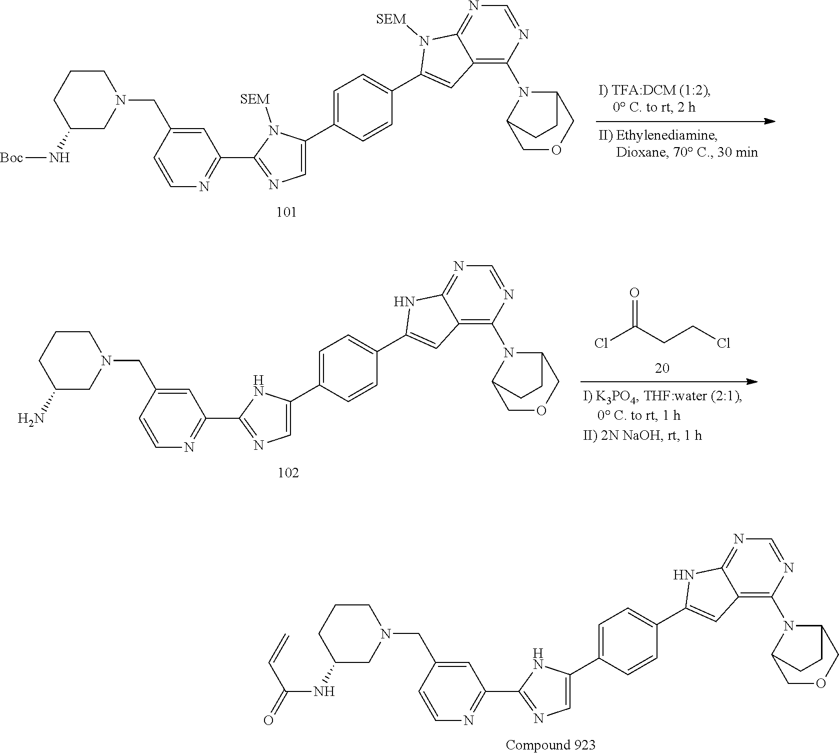 [SMILES: CC(C)(C)OC(=O)N[C@@H]1CCCN(Cc2ccnc(-c3ncc(-c4ccc(-c5cc6c(N7C8CCC7COC8)ncnc6n5COCC[Si](C)(C)C)cc4)n3COCC[Si](C)(C)C)c2)C1 ; N[C@@H]1CCCN(Cc2ccnc(-c3ncc(-c4ccc(-c5cc6c(N7C8CCC7COC8)ncnc6[nH]5)cc4)[nH]3)c2)C1 ; O=C(Cl)CCCl ; C=CC(=O)N[C@@H]1CCCN(Cc2ccnc(-c3ncc(-c4ccc(-c5cc6c(N7C8CCC7COC8)ncnc6[nH]5)cc4)[nH]3)c2)C1]

### Synthesis of methyl 4-(((methylsulfonyl)oxy)methyl)picolinate (94)

To a stirred solution of methyl 4-(hydroxymethyl)picolinate 1 (3 g, 17.9 mmol) in DCM (30 mL) were added Et₃N (7.46 mL, 53.8 mmol) and MsCl (1.67 mL, 21.5 mmol) at 0° C. under N₂ atmosphere. The reaction mixture was stirred at RT for 3 h. After completion of reaction (TLC and LCMS monitoring), the reaction mixture was diluted with ice cold water (50 mL) and extracted with DCM (2×50 mL). The combined organic layer was dried over Na₂SO₄, filtered and evaporated under reduced pressure to get the desired compound as a yellow solid 94 (4.8 g; crude) which was directly used in next step without further purification.

LCMS=[M+H]⁺: 246.27; Purity=88%.

### Synthesis of methyl (R)-4-((3-((tert-butoxycarbonyl)amino)piperidin-1-yl)methyl)picolinate (95)

To a stirred solution of methyl 4-(((methylsulfonyl)oxy)methyl)picolinate 94 (4.8 g, 19.6 mmol) and tert-butyl (R)-piperidin-3-ylcarbamate 56 (7.84 g, 39.1 mmol) in DMF (100 mL) was added K₂CO₃ (13.5 g, 97.9 mmol) at RT and stirred at 120° C. for 2 h. After completion of reaction (TLC monitoring), the reaction mass was poured into ice-cold water (100 mL) and extracted with EtOAc (3×100 mL). The combined organic layer was washed with ice cold brine solution (2×100 mL), dried over Na₂SO₄, filtered and evaporated under reduced pressure. The crude was purified by flash chromatography (silica gel 100-200 M, 80 g SNAP) using 15-20% EtOAc in heptane to get the desired compound as light yellow semisolid 95 (4 g, 58%).

LCMS=[M+H]⁺: 350.34; Purity=97%.

### Synthesis of tert-butyl (R)-(1-((2-formylpyridin-4-yl)methyl)piperidin-3-yl)carbamate (96)

To a stirred solution of methyl (R)-4-((3-((tert-butoxycarbonyl)amino)piperidin-1-yl)methyl)picolinate 95 (2.2 g, 6.3 mmol) in THF (25 mL) was added DIBAL-H (7.6 mL, 7.56 mmol) dropwise at −78° C. under N₂ atmosphere and stirred for 1 h. After completion of reaction (TLC monitoring), the reaction mixture was cooled to 0° C. and quenched with water (8 mL) and 10% aq. NaOH (8 mL) and stirred for 15 minutes. The reaction mixture was filtered through Celite pad and washed with EtOAc. The filtrate was diluted with water (50 mL) and extracted with EtOAc (2×100 mL). The combined organic layer was dried over anhydrous Na₂SO₄, filtered and concentrated under reduced pressure to get the desired compound as a yellow semisolid 96 (2 g; crude) which was used in next step directly without any purification.

LCMS=[M+H]⁺: 320.47; Purity=70%

### Synthesis of tert-butyl (R)-(1-((2-(5-(4-bromophenyl)-1H-imidazol-2-yl)pyridin-4-yl)methyl)piperidin-3-yl)carbamate (98)

To a stirred solution of tert-butyl (R)-(1-((2-formylpyridin-4-yl)methyl)piperidin-3-yl)carbamate 96 (2 g, 6.26 mmol) in MeOH (30 mL) was added and NH₄OAc (1.45 g, 18.8 mmol) and stirred at RT for 30 min. Then, 2-(4-bromophenyl)-2-oxoacetaldehyde 97 (1.33 g, 6.26 mmol) dissolved with MeOH (5 mL) was added to the reaction mixture and stirred the reaction mixture at 60° C. for 1 h. After completion of reaction (TLC and LCMS monitoring), the reaction mixture was concentrated under reduced pressure. The crude was diluted with water (50 mL) and extracted with EtOAc (3×50 mL). The combined organic layer was dried over Na₂SO₄, filtered and evaporated under reduced pressure to get the desired compound as a brown solid 98 (2 g; crude) which was used in next step directly without any purification.

LCMS=[M+H]⁺: 514.30; Purity=65%

### Synthesis of tert-butyl (R)-(1-((2-(5-(4-bromophenyl)-1-((2-(trimethylsilyl)ethoxy)methyl)-1H-imidazol-2-yl)pyridin-4-yl)methyl)piperidin-3-yl)carbamate (99)

To a stirred solution of tert-butyl (R)-(1-((2-(5-(4-bromophenyl)-1H-imidazol-2-yl)pyridin-4-yl)methyl)piperidin-3-yl)carbamate 98 (2 g, 4.88 mmol) in THF (30 mL) was added NaH (319 mg, 7.32 mmol; 60% dispersion in mineral oil) at 0° C. under N₂ atmosphere and stirred at RT for 15 min. Then, SEM-C1 (1.04 mL, 5.85 mmol) was added dropwise to the reaction mixture at 0° C. and stirred at RT for 30 min. After completion of reaction (TLC and LCMS monitoring), the reaction mixture was diluted with ice cold water (50 mL) and extracted with EtOAc (3×50 mL). The combined organic layer was dried over Na₂SO₄, filtered and evaporated under reduced pressure. The crude was purified by column chromatography (12 g SNAP, silica gel 100-200 M) using 25-30% EtOAc in heptane to get the desired compound as a sticky yellow solid 99 (900 mg, 27%).

LCMS=[M+H]⁺: 642.58; Purity=85%

### Synthesis of tert-butyl (R)-(1-((2-(5-(4-(4,4,5,5-tetramethyl-1,3,2-dioxaborolan-2-yl)phenyl)-1-((2-(trimethylsilyl)ethoxy)methyl)-1H-imidazol-2-yl)pyridin-4-yl)methyl)piperidin-3-yl)carbamate (100)

To a stirred solution of tert-butyl (R)-(1-((2-(5-(4-bromophenyl)-1-((2-(trimethylsilyl)ethoxy)methyl)-1H-imidazol-2-yl)pyridin-4-yl)methyl)piperidin-3-yl)carbamate 99 (900 mg, 1.56 mmol) in 1,4-dioxane (10 mL) were added B₂Pin₂ (593 mg, 2.33 mmol) and KOAc (458 mg, 4.67 mmol) at RT. The reaction mixture was degassed with N₂ gas for 15 min. Then, PdCl₂(dppf)·DCM (127 mg, 0.156 mmol) was added to the reaction mixture and stirred at 100° C. for 1 h. After the completion of reaction (TLC and LCMS monitoring), the reaction mixture was filtered through Celite pad and washed with EtOAc. Then the filtrate was evaporated under reduced pressure to get the desired compound as a brown semisolid 100 (1.5 g; crude) which was used in the next step directly without further purification.

LCMS=[M+H]⁺: 690.54; Purity=55%.

### Synthesis of tert-butyl ((3R)-1-((2-(5-(4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)-1-((2-(trimethylsilyl)ethoxy)methyl)-1H-imidazol-2-yl)pyridin-4-yl)methyl)piperidin-3-yl)carbamate (101)

To a stirred solution of (1R,5S)-8-(6-iodo-7-((2-(trimethylsilyl)ethoxy)methyl)-7H-pyrrolo[2,3-d]pyrimidin-4-yl)-3-oxa-8-azabicyclo[3.2.1]octane 84 (400 mg, 0.822 mmol) in 1,4-dioxane (20 mL), tert-butyl (R)-(1-((2-(5-(4-(4,4,5,5-tetramethyl-1,3,2-dioxaborolan-2-yl)phenyl)-1-((2-(trimethylsilyl)ethoxy)methyl)-1H-imidazol-2-yl)pyridin-4-yl)methyl)piperidin-3-yl)carbamate 100 (1.5 g, 2.47 mmol) and aqueous solution of K₂CO₃ (341 mg, 2.47 mmol) in water (5 mL) were added at RT. The reaction mixture was degassed with N₂ gas for 15 min. Then, PdCl₂(dppf)·DCM (67.1 mg, 0.082 mmol) was added to the reaction mixture stirred at 100° C. for 2 h. After the completion of reaction (TLC and LCMS monitoring), the reaction mixture was filtered through Celite pad and washed with EtOAc. The filtrate was diluted with water (50 mL) and extracted with EtOAc (3×50 mL). The combined organic layer was dried over Na₂SO₄, filtered and evaporated under reduced pressure. The crude was purified by column chromatography (12 g SNAP, silica gel 100-200 M) using 35-40% EtOAc in heptane to get the desired compound as a sticky brown 101 (400 mg, 25%).

LCMS=[M+H]⁺: 922.54; Purity=71%

### Synthesis of (3R)-1-((2-(5-(4-(4-(3-oxa-8-azabicyclo[3.2.1]octan-8-yl)-7H-pyrrolo[2,3-d]pyrimidin-6-yl)phenyl)-1H-imidazol-2-yl)pyridin-4-yl)methyl)piperidin-3-amine (102)

To a stirred solution of tert-butyl ((3R)-1-((2-(5-(4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)-1-((2-(trimethylsilyl)ethoxy)methyl)-1H-imidazol-2-yl)pyridin-4-yl)methyl)piperidin-3-yl)carbamate 101 (180 mg, 0.141 mmol) in DCM (3 mL) was added TFA (1.5 mL) dropwise to the reaction mixture at 0° C. and stirred at RT for 2 h. After consumed of starting material (TLC monitoring), the reaction mixture was concentrated under reduced pressure. The crude was dissolved in 1,4-dioxane (3 mL) and 1,2-ethanediamine (14 μL, 0.211 mmol) was added to the reaction mixture and stirred at 70° C. for 30 min. After completion of reaction (LCMS monitoring), the reaction mixture was diluted with water (20 mL) and extracted with 25% IPA in CHCl₃ (3×20 mL). The combined organic layer was dried over Na₂SO₄, filtered and concentrate under reduced pressure to get the desired compound as a yellow solid 102 (70 mg; crude) which was used in next step directly without any purification.

LCMS=[M+H]⁺: 562.55; Purity=65%

### Synthesis of N-((3R)-1-((2-(5-(4-(4-(3-oxa-8-azabicyclo[3.2.1]octan-8-yl)-7H-pyrrolo[2,3-d]pyrimidin-6-yl)phenyl)-1H-imidazol-2-yl)pyridin-4-yl)methyl)piperidin-3-yl)acrylamide (Compound 923)

To a stirred solution of (3R)-1-((2-(5-(4-(4-(3-oxa-8-azabicyclo[3.2.1]octan-8-yl)-7H-pyrrolo[2,3-d]pyrimidin-6-yl)phenyl)-1H-imidazol-2-yl)pyridin-4-yl)methyl)piperidin-3-amine 102 (70 mg, 0.125 mmol) in THF (3 mL) was added K₃PO₄ (52.9 mg, 0.249 mmol) dissolved in water (1.5 mL, 83.3 mmol) at 0° C. Then, 3-chloropropionyl chloride 20 (19 mg, 0.150 mmol) dissolved with THF (0.5 mL) was added dropwise to the reaction mixture at 0° C. and stirred at for 1 h. After consumed of starting material (monitoring by TLC), 2N aq. NaOH (0.7 mL, 1.5 mmol) was added dropwise to the reaction mixture at 0° C. and stirred the reaction mixture at RT for 1 h. After completion of reaction (LCMS monitoring), the reaction mixture was poured into water (10 mL) and extracted with 25% IPA in CHCl₃ (3×10 mL). The combined organic layer was dried over Na₂SO₄, filtered and concentrated under reduced pressure. The crude was purified by RP-HPLC [5-95% 5 mM ammonium bicarbonate in water/ACN; Column: X select phenyl hexyl (19*250) 5μ] to get the desired Compound 923 (12 mg, 15%).

¹H NMR (400 MHz, DMSO-d₆): δ 12.96 (s, 1H), 12.21 (s, 1H), 8.55 (d, J=4.8 Hz, 1H), 8.18 (s, 1H), 8.07 (s, 1H), 8.03-7.94 (m, 5H), 7.83 (s, 1H), 7.34 (d, J=4.8 Hz, 1H), 7.15 (d, J=1.6 Hz, 1H), 6.26-6.18 (m, 1H), 6.04 (dd, J=16.8 Hz, 2 Hz, 1H), 5.54 (dd, J=10 Hz, 2 Hz, 1H), 4.93 (brs, 2H), 3.84-3.83 (m, 1H), 3.72-3.70 (m, 2H), 3.65-3.59 (m, 4H), 2.82-2.79 (m, 1H), 2.72-2.68 (m, 1H), 2.05-1.90 (m, 6H), 1.78-1.69 (m, 2H), 1.56-1.53 (m, 1H), 1.26-1.21 (m, 1H).

LCMS=[M+H]⁺: 616.28; Purity=97.67%.

### Synthesis of 2-(4-bromophenyl)-2-oxoacetaldehyde (97)

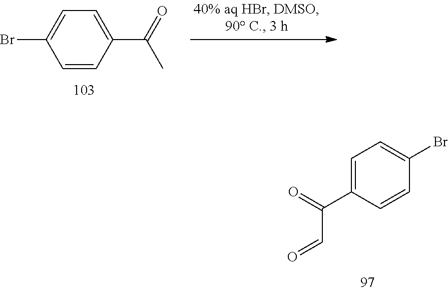

To a stirred solution of 1-(4-bromophenyl) ethan-1-one 103 (2 g, 10 mmol) in DMSO (20 mL) was added HBr (2.32 mL, 20.1 mmol; 47% w/v in water) at RT and stirred at 90° C. for 3 h. After completion of reaction (TLC monitoring), the reaction mixture was poured into water (100 mL) and stirred at 55° C. for 30 min. The yellow solid was collected by filtration and washed with mixture of acetone: water (1 mL: 19 mL) to get the desired compound as a yellow solid (1.3 g; crude) which was used in the next step directly without any purification.

¹H NMR (400 MHz, CDCl₃-d₆): δ 9.63 (s, 1H), 7.99 (d, J=8.4 Hz, 2H), 7.67 (d, J=8 Hz, 2H).

### Example 75

### Compound 924

### Preparation of 2-(azetidin-1-ylmethyl)-1-(4-(2,2,2-trifluoro-1-((4-(4-morpholino-7H-pyrrolo[2,3-d]pyrimidin-6-yl)phenyl)amino)ethyl)-[1,4′-bipiperidin]-1′-yl)prop-2-en-1-one (Compound 924)

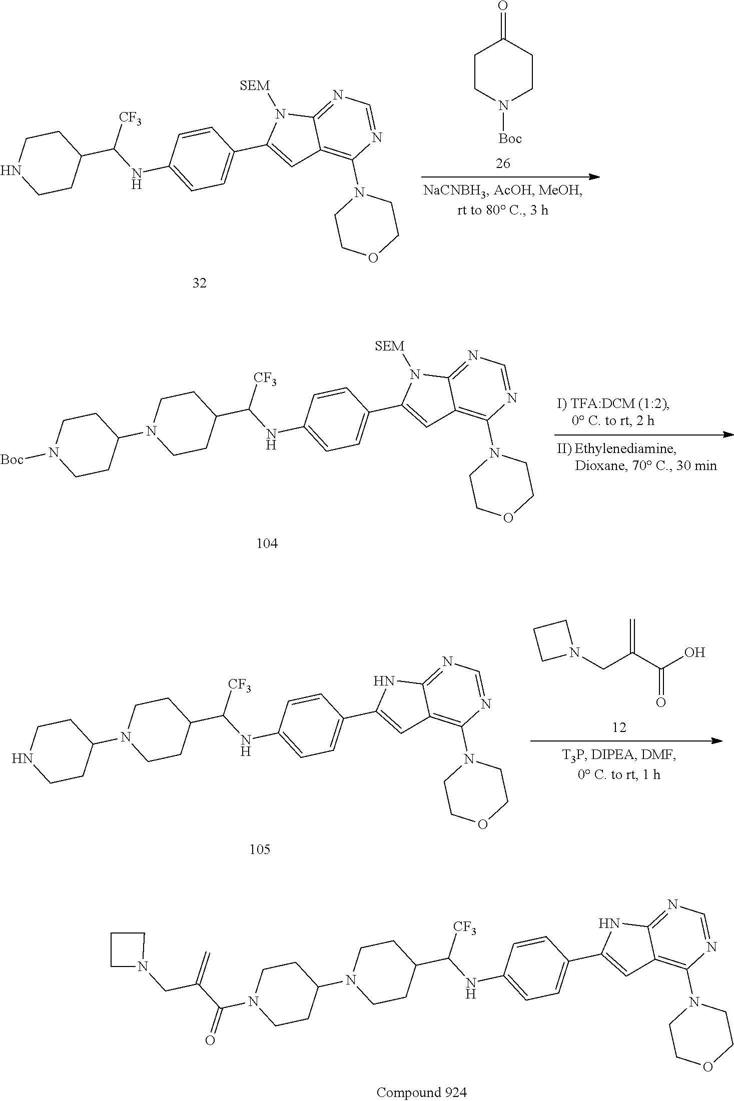

### Synthesis of tert-butyl 4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)-[1,4′-bipiperidine]-1′-carboxylate (104)

To a stirred solution of 4-(4-morpholino-7-((2-(trimethylsilyl)ethoxy)methyl)-7H-pyrrolo[2,3-d]pyrimidin-6-yl)-N-(2,2,2-trifluoro-1-(piperidin-4-yl)ethyl)aniline 32 (500 mg, 0.864 mmol) in MeOH (10 mL) were added tert-butyl 4-oxopiperidine-1-carboxylate 26 (422 mg, 2.12 mmol) and AcOH (0.9 mL, 1.70 mmol) at 0° C. under N₂ atmosphere and stirred for 30 min. Then, NaCNBH₃ (101 mg, 1.69 mmol) was added to the reaction mixture and stirred at 80° C. for 3 h. After completion of reaction (TLC and LCMS monitoring), the reaction mixture was quenched with sat. NaHCO₃ (50 mL) and extracted with 25% IPA in CHCl₃ (3×50 mL). The combined organic layer was dried over Na₂SO₄, filtered and concentrated under reduced pressure. The crude was purified by flash chromatography (silica gel 100-200 M, 12 g SNAP) using 5-10% MeOH in DCM to get the desired compound as a pale yellow solid 104 (800 mg, 90%).

LCMS=[M+H]⁺: 774.45; Purity=75%.

### Synthesis of N-(1-([1,4′-bipiperidin]-4-yl)-2,2,2-trifluoroethyl)-4-(4-morpholino-7H-pyrrolo[2,3-d]pyrimidin-6-yl)aniline (105)

To a stirred solution of tert-butyl 4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)-[1,4′-bipiperidine]-1′-carboxylate 104 (800 mg, 1.03 mmol) in DCM (8 mL) was added TFA (4 mL) dropwise at 0° C. The resulting reaction mixture was stirred at RT for 2 h. After consumed of starting material (LCMS monitoring), the reaction mixture was concentrated under reduced pressure. The crude was dissolved in 1,4-dioxane (5 mL) followed by, 1,2-ethanediamine (0.1 mL, 1.55 mmol) was added to the reaction mixture at RT and stirred at 70° C. for 30 min. After completion of reaction (LCMS monitoring), the reaction mixture was diluted with water (50 mL) and extracted with 25% IPA in CHCl₃ (3×50 mL). The combined organic layer was dried over Na₂SO₄, filtered and concentrated under reduced pressure to get the title compound as a pale yellow solid 105 (400 mg; crude) which was used in next step directly without further purification.

LCMS=[M−H]: 542.27; Purity=50%.

### Synthesis of 2-(azetidin-1-ylmethyl)-1-(4-(2,2,2-trifluoro-1-((4-(4-morpholino-7H-pyrrolo[2,3-d]pyrimidin-6-yl)phenyl)amino)ethyl)-[1,4′-bipiperidin]-1′-yl)prop-2-en-1-one (Compound 924)

To a stirred solution of N-(1-([1,4′-bipiperidin]-4-yl)-2,2,2-trifluoroethyl)-4-(4-morpholino-7H-pyrrolo[2,3-d]pyrimidin-6-yl)aniline 105 (200 mg, 0.668 mmol) in DMF (5 mL) were added 2-(azetidin-1-ylmethyl)acrylic acid 12 (78 mg, 0.553 mmol), DIPEA (0.19 mL, 1.1 mmol) and T3P (0.22 mL 0.736 mmol; 50% w/v in EtOAc) sequentially at 0° C. under N₂ atmosphere. The reaction mixture was stirred at RT for 1 h. After completion of reaction (TLC monitoring), the reaction mixture was poured into ice cold water (50 mL) and extracted with 25% IPA in CHCl₃ (3×50 mL). The combined organic layer was dried over Na₂SO₄, filtered and concentrated under reduced pressure. The crude was purified by RP-HPLC [20-80% 5 mM Ammonium Bicarbonate in Water/ACN; Column: X Select CSH C18 (19*250) 10μ] to get the title compound Compound 924 (10 mg, 4%).

¹H NMR (400 MHz, DMSO-d₆): δ 11.95 (s, 1H), 8.13 (s, 1H), 7.63 (d, J=8.4 Hz, 2H), 6.87-6.84 (m, 3H), 6.05 (d, J=9.6 Hz, 1H), 5.22 (s, 1H), 5.00 (s, 1H), 4.31-4.28 (m, 2H), 3.87-3.83 (m, 5H), 3.74-3.72 (m, 4H), 3.09-3.06 (m, 6H), 2.95-2.85 (m, 3H), 2.59-2.56 (m, 1H), 2.45-2.42 (m, 1H), 2.13-2.06 (m, 2H), 1.97-1.90 (m, 2H), 1.73-1.66 (m, 5H), 1.49-1.42 (m, 2H), 1.39-1.30 (m, 2H).

LCMS=[M+H]⁺: 667.31; Purity=90.29%.

### Example 76

### Compound 927

### Preparation of 1-(4-(1-((4-(4-(3-oxa-8-azabicyclo[3.2.1]octan-8-yl)-7H-pyrrolo[2,3-d]pyrimidin-6-yl)phenyl)amino)-2,2,2-trifluoroethyl)piperidin-1-yl)prop-2-en-1-one (Compound 927)

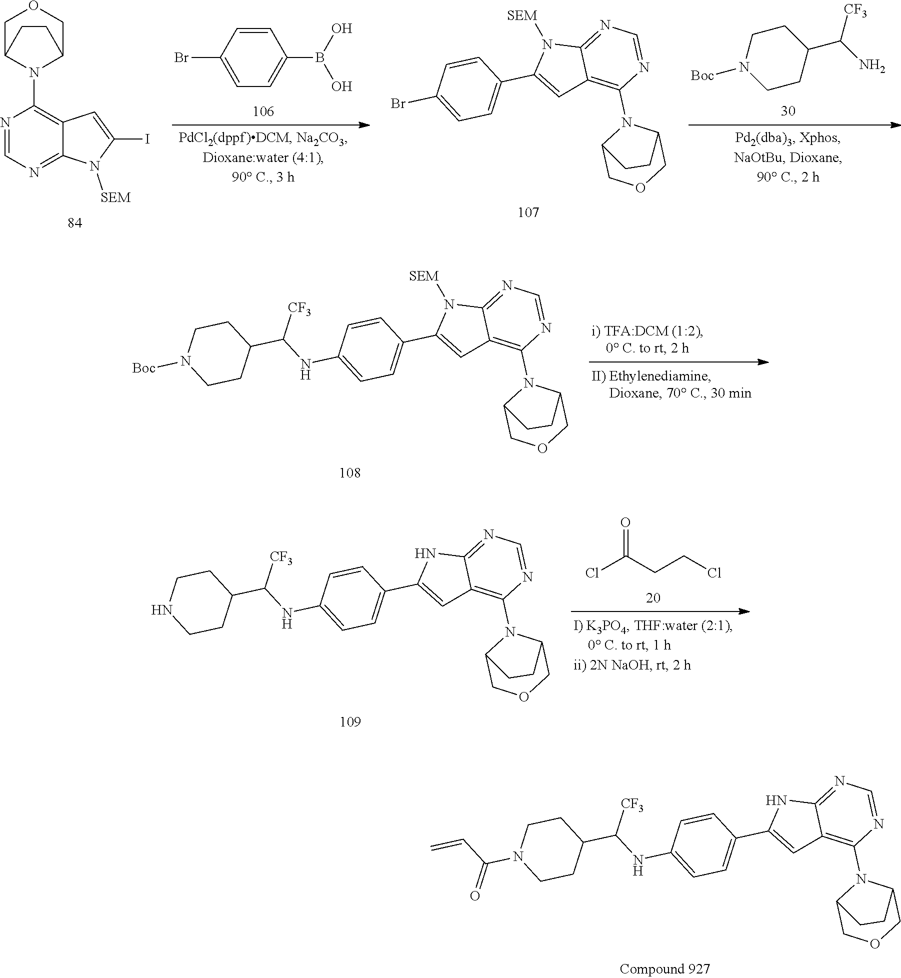

### Synthesis of 8-(6-(4-bromophenyl)-7-((2-(trimethylsilyl)ethoxy)methyl)-7H-pyrrolo[2,3-d]pyrimidin-4-yl)-3-oxa-8-azabicyclo[3.2.1]octane (107)

To a stirred solution of [2-({2-iodo-4-(3-oxa-8-azabicyclo[3.2.1]oct-8-yl)-1H-1,5,7-triazainden-1-yl}methoxy)ethyl]tris(methyl) silane 84 (4 g, 8.22 mmol) in 1,4-dioxane (40 mL) was added aqueous solution of Na2CO₃ (2.61 g, 24.7 mmol) dissolved in H₂O (10 mL) followed by (4-bromophenyl) boronic acid 106 (1.98 g, 9.87 mmol) was added to the reaction mixture at RT. The reaction mixture was degassed with N₂ gas for 15 min. Then, PdCl₂(dppf)·DCM (671 mg, 0.822 mmol) was added to the reaction mixture and stirred at 90° C. for 3 h. After the completion of reaction (TLC and LCMS monitoring), the reaction mixture was filtered through Celite pad and washed with EtOAc. The filtrate was diluted with water (100 mL) and extracted with EtOAc (3×100 mL). The combined organic layer was dried over Na₂SO₄, filtered and evaporated under reduced pressure. The crude was purified by column chromatography (silica gel 100-200 M, 80 g SNAP) using 15-20% EtOAc in heptane to get the title compound as an off white solid 107 (2.8 g, 44%).

LCMS=[M+H]⁺: 517.19; Purity=75%

### Synthesis of tert-butyl 4-(1-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)-2,2,2-trifluoroethyl)piperidine-1-carboxylate (108)

To a stirred solution of tert-butyl 4-(1-aminoethyl)-1-piperidinecarboxylate 30 (1.2 g, 5.26 mmol) in 1,4-dioxane (20 mL) were added and [2-({2-(p-bromophenyl)-4-(3-oxa-8-azabicyclo[3.2.1]oct-8-yl)-1H-1,5,7-triazainden-1-yl}methoxy)ethyl]tris(methyl) silane 107 (2.71 g, 5.26 mmol) and NaO^{t}Bu (758 mg, 7.88 mmol) at RT. The reaction mixture was degassed with N₂ gas for 10 min, followed by XPhos (501 mg, 1.05 mmol) and Pd₂(dba)₃ (425 mg, 0.526 mmol) were added to the reaction mixture and stirred at 90° C. for 2 h. After completion of reaction (TLC monitoring), the reaction mass was poured into ice cold water (50 mL) and extracted with EtOAc (3×100 mL). The combined organic layer was washed with brine solution (2×50 mL), dried over Na₂SO₄, filtered and concentrated under reduced pressure. The crude was purified by column chromatography using 30-35% EtOAc in heptane to get the title compound as a pale yellow solid 108 (2.7 g, 77%).

LCMS=[M+H]⁺: 717.37; Purity=62%

### Synthesis of 4-(4-(3-oxa-8-azabicyclo[3.2.1]octan-8-yl)-7H-pyrrolo[2,3-d]pyrimidin-6-yl)-N-(2,2,2-trifluoro-1-(piperidin-4-yl)ethyl)aniline (109)

To a stirred solution of tert-butyl 4-(2,2,2-trifluoro-1-{p-[4-(3-oxa-8-azabicyclo[3.2.1]oct-8-yl)-1-{[2-(trimethylsilyl) ethoxy]methyl}-1H-1,5,7-triazainden-2-yl]phenylamino}ethyl)-1-piperidinecarboxylate 108 (2.7 g, 3.77 mmol) in DCM (20 mL) was added TFA (10 mL) dropwise at 0° C. and stirred at RT for 2 h. After consumed of starting material (TLC monitoring), the reaction mixture was concentrated under reduced pressure. The crude was dissolved in 1,4-dioxane (20 mL) and 1,2-ethanediamine (0.5 mL, 7.53 mmol) was added at RT and stirred at 70° C. for 30 min. After completion of reaction (LCMS monitoring), the reaction mixture was diluted with 25% IPA in CHCl₃ (100 mL) and washed with ice cold water (2×50 mL). The organic layer was dried over Na₂SO₄, filtered and concentrated under reduced pressure to get the title compound as an off white solid 109 (1.6 g; crude) which was used in next step directly without further purification.

LCMS=[M+H]⁺: 487.22

### Synthesis of 1-(4-(1-((4-(4-(3-oxa-8-azabicyclo[3.2.1]octan-8-yl)-7H-pyrrolo[2,3-d]pyrimidin-6-yl)phenyl)amino)-2,2,2-trifluoroethyl)piperidin-1-yl)prop-2-en-1-one (Compound 927)

To a stirred solution of [2,2,2-trifluoro-1-(4-piperidyl)ethyl](p-{4-(3-oxa-8-azabicyclo[3.2.1]oct-8-yl)-1H-1,5,7-triazainden-2-yl}phenyl)amine 109 (1.5 g, 3.08 mmol) in THF (20 mL) was added K₃PO₄ (1.31 g, 6.17 mmol) dissolved in water (10 mL) at 0° C. Then, 3-chloropropionyl chloride 20 (470 mg, 3.7 mmol) dissolved in THF (2 mL) was added dropwise to the reaction mixture at 0° C. The reaction mixture was stirred at RT for 1 h. After consumed of starting material (monitoring by TLC), 2N aq. NaOH (18.5 mL, 37 mmol) was added dropwise to the reaction mixture at 0° C. and stirred at RT for 2 h. After completion of reaction (LCMS monitoring), the reaction mixture was diluted with water (50 mL) and extracted with 2-MeTHF (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get the title compound Compound 927 (250 mg, 15%).

¹H NMR (400 MHz, DMSO-d₆): δ 11.93 (s, 1H), 8.13 (s, 1H), 7.64 (d, J=8.8 Hz, 2H), 6.86-6.75 (m, 4H), 6.09-6.06 (m, 2H), 5.64 (t, J=7.2 Hz, 1H), 4.87 (s, 2H), 4.49-4.37 (m, 2H), 4.10 (t, J=12.8 Hz, 1H), 3.69 (d, J=10.8 Hz, 2H), 3.60 (d, J=10.8 Hz, 2H), 3.04 (q, J=12.4 Hz, 1H), 2.66-2.6 (m, 1H), 2.09-1.94 (m, 5H), 1.77-1.74 (m, 2H), 1.42-1.27 (m, 2H).

LCMS=[M+H]⁺: 541.23; Purity=95.14%.

### Example 77

### Compound 925 and Compound 926

### Preparation of 1-(4-((1R)-1-((4-(4-(3-oxa-8-azabicyclo[3.2.1]octan-8-yl)-7H-pyrrolo[2,3-d]pyrimidin-6-yl)phenyl)amino)-2,2,2-trifluoroethyl)piperidin-1-yl)prop-2-en-1-one (Compound 925) and 1-(4-((1S)-1-((4-(4-(3-oxa-8-azabicyclo[3.2.1]octan-8-yl)-7H-pyrrolo[2,3-d]pyrimidin-6-yl)phenyl)amino)-2,2,2-trifluoroethyl)piperidin-1-yl)prop-2-en-1-one (Compound 926)

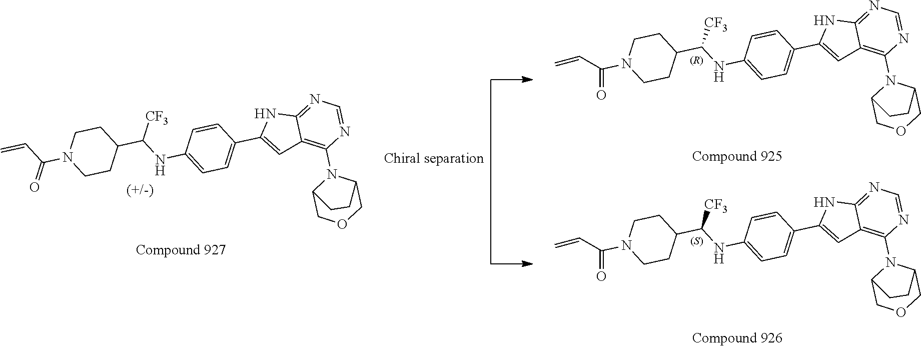

Chiral separation of Compound 927 resulted in both enantiomers 1-(4-((1R)-1-((4-(4-(3-oxa-8-azabicyclo[3.2.1]octan-8-yl)-7H-pyrrolo[2,3-d]pyrimidin-6-yl)phenyl)amino)-2,2,2-trifluoroethyl)piperidin-1-yl)prop-2-en-1-one (Compound 925) and 1-(4-((1S)-1-((4-(4-(3-oxa-8-azabicyclo[3.2.1]octan-8-yl)-7H-pyrrolo[2,3-d]pyrimidin-6-yl)phenyl)amino)-2,2,2-trifluoroethyl)piperidin-1-yl)prop-2-en-1-one (Compound 926)

Note: Chirality arbitrarily assigned

Methods for SFC purification: Column: Chiralcel OX-H (30×250 mm; 5μ); Mobile Phase: A) CO₂ B) 0.2% Et3N in MeOH; Flow rate: Isocratic 100 mL/Min, wavelength: 334 nm

Analytical data of Compound 925:

¹H NMR (400 MHz, DMSO-d₆): δ 11.94 (s, 1H), 8.12 (s, 1H), 7.64 (d, J=8.4 Hz, 2H), 6.86-6.75 (m, 4H), 6.09-6.05 (m, 2H), 5.65-5.63 (m, 1H), 4.87 (s, 2H), 4.50-4.37 (m, 2H), 4.13-4.08 (m, 1H), 3.69 (d, J=10.8 Hz, 2H), 3.60 (d, J=10.8 Hz, 2H), 3.09-2.99 (m, 1H), 2.64-2.62 (m, 1H), 2.09-1.94 (m, 5H), 1.79-1.73 (m, 2H), 1.41-1.28 (m, 2H).

LCMS=[M+H]⁺: 541.23; Purity=98.61% and ee=99.92%

Analytical data of Compound 926:

¹H NMR (400 MHz, DMSO-d₆): δ 11.94 (s, 1H), 8.12 (s, 1H), 7.64 (d, J=8.8 Hz, 2H), 6.86-6.75 (m, 4H), 6.09-6.05 (m, 2H), 5.66-5.62 (m, 1H), 4.87 (s, 2H), 4.50-4.37 (m, 2H), 4.12-4.07 (m, 1H), 3.69 (d, J=10.8 Hz, 2H), 3.60 (d, J=10.4 Hz, 2H), 3.09-2.99 (m, 1H), 2.66-2.59 (m, 1H), 2.09-1.94 (m, 5H), 1.77-1.73 (m, 2H), 1.41-1.28 (m, 2H).

LCMS=[M+H]⁺: 541.23; Purity=98.03% and ee=96%

### Example 78

### Compound 928

### Preparation of (R)—N-(1-(5-((4-(4-morpholino-7H-pyrrolo[2,3-d]pyrimidin-6-yl)phenyl)amino)pyridin-2-yl)piperidin-3-yl)acrylamide (Compound 928)

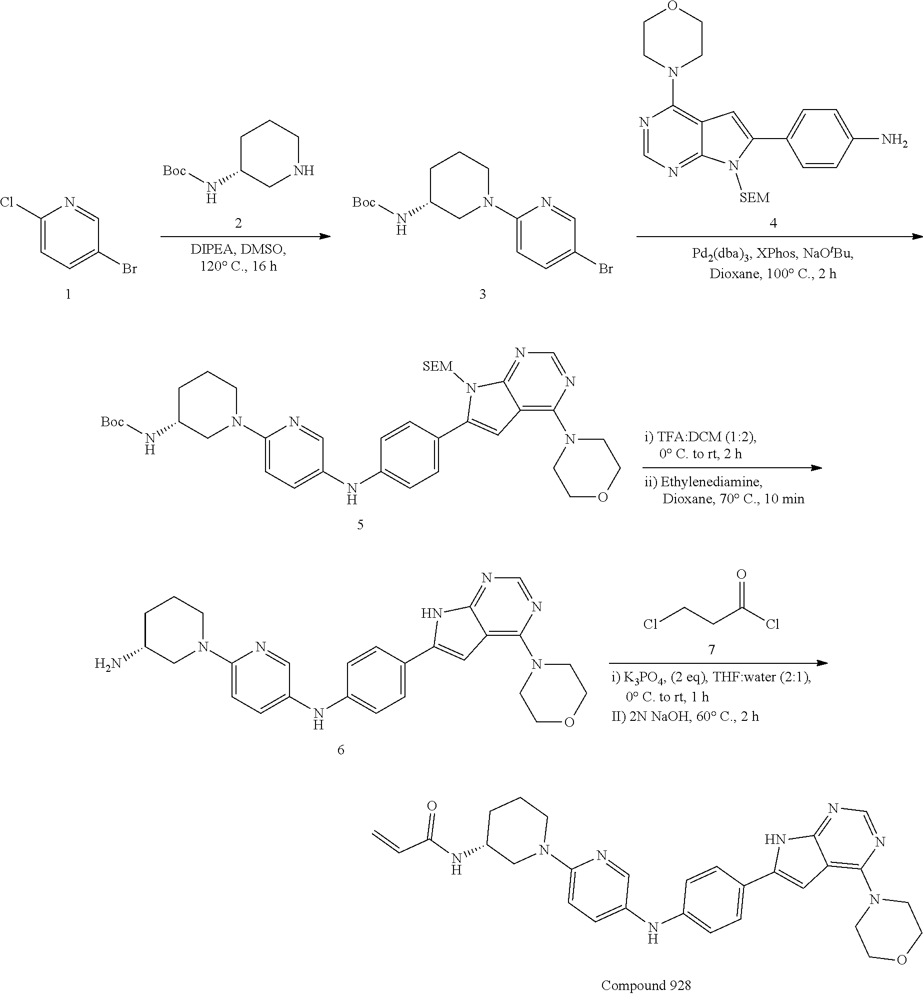

### Synthesis of tert-butyl (R)-(1-(5-bromopyridin-2-yl)piperidin-3-yl)carbamate (3)

To a stirred solution of 5-bromo-2-chloropyridine 1 (1.15 g, 5.99 mmol) and tert-butyl (R)-piperidin-3-ylcarbamate 2 (1 g, 4.99 mmol) in DMSO (20 mL) was added DIPEA (2.6 mL, 15 mmol) at RT under N₂ atmosphere. The resulting reaction mixture was stirred at 120° C. for 16 h. After completion of reaction (monitored by TLC and LCMS), the reaction mixture was poured into ice cold water (100 mL) and extracted with EtOAc (3×100 mL). The combined organic layer was washed with ice cold brine (2×50 mL), dried over Na2SO₄, filtered and concentrated under reduced pressure. The crude was purified by flash column chromatography (silica gel 100-200M, 12 g SNAP) using 8-10% EtOAc in heptane to get the title compound as a white solid 3 (1.1 g, 62%).

LCMS=[M+H]⁺: 356.06; Purity=98%.

### Synthesis of tert-butyl (R)-(1-(5-((4-(4-morpholino-7-((2-(trimethylsilyl)ethoxy)methyl)-7H-pyrrolo[2,3-d]pyrimidin-6-yl)phenyl)amino)pyridin-2-yl)piperidin-3-yl)carbamate (5)

To a stirred solution of tert-butyl (R)-(1-(5-bromopyridin-2-yl)piperidin-3-yl)carbamate 3 (1 g, 2.81 mmol) and 4-(4-morpholino-7-((2-(trimethylsilyl)ethoxy)methyl)-7H-pyrrolo[2,3-d]pyrimidin-6-yl)aniline 4 (1.19 g, 2.81 mmol) in 1,4-dioxane (15 mL) was added NaOtBu (405 mg, 4.21 mmol). The resulting reaction mixture was degassed with N₂ gas for 10 min, followed by XPhos (200 mg, 0.421 mmol) and Pd₂(dba)₃ (227 mg, 0.281 mmol) were added to the reaction mixture at RT. The reaction mixture was stirred at 100° C. for 2 h. After completion of reaction (monitored by TLC and LCMS), the reaction mixture was filtered through celite pad and washed with EtOAc (100 mL). The filtrate was washed with water (2×50 mL). The organic layer was separated, dried over Na₂SO₄, filtered and evaporated under reduced pressure. The crude was purified by flash column chromatography (silica gel 100-200 M, 12 g SNAP) using 20-30% EtOAc in heptane to get the title compound as a light yellow solid 5 (1.3 g, 66%).

LCMS=[M+H]⁺: 701.51; Purity=70%.

### Synthesis of (R)-6-(3-aminopiperidin-1-yl)-N-(4-(4-morpholino-7H-pyrrolo[2,3-d]pyrimidin-6-yl)phenyl)pyridin-3-amine (6)

To a stirred solution of tert-butyl (R)-(1-(5-((4-(4-morpholino-7-((2-(trimethylsilyl)ethoxy)methyl)-7H-pyrrolo[2,3-d]pyrimidin-6-yl)phenyl)amino)pyridin-2-yl)piperidin-3-yl)carbamate 5 (1.3 g, 1.85 mmol) in DCM (20 mL) was added TFA (10 mL) dropwise under N₂ atmosphere at 0° C. The reaction mixture was stirred at RT for 2 h. After completion of reaction (monitored by TLC and LCMS), the reaction mixture was concentrated under reduced pressure, triturated with Et2O and dried under reduced pressure. The obtained crude was dissolved in 1,4-dioxane (10 mL) and ethylenediamine (0.2 mL, 2.94 mmol) was added to the reaction mixture and stirred at 70° C. for 10 min. After completion of reaction (monitored by LCMS), the reaction was poured into ice cold water (50 mL) and extracted with 25% IPA in CHCl₃ (3×50 mL). The combined organic layer was dried over Na₂SO₄, filtered and concentrated under reduced pressure to get the title compound as brown solid 6 (700 mg; crude) which was used in next step directly without further purification.

LCMS=[M+H]⁺: 471.31; Purity=65%.

### Synthesis of (R)—N-(1-(5-((4-(4-morpholino-7H-pyrrolo[2,3-d]pyrimidin-6-yl)phenyl)amino)pyridin-2-yl)piperidin-3-yl)acrylamide (Compound 928)

To an ice cold stirred solution of (R)-6-(3-aminopiperidin-1-yl)-N-(4-(4-morpholino-7H-pyrrolo[2,3-d]pyrimidin-6-yl)phenyl)pyridin-3-amine 6 (700 mg, 1.49 mmol) in THF (10 mL) was added K₃PO₄ (630 mg, 2.98 mmol) dissolved in water (5 mL) under N₂ atmosphere and stirred for 5 min. Then added stock solution of 3-chloropropionyl chloride 7 (170 μL, 1.79 mmol) in THF (1 mL) drop wise to the reaction mixture at 0° C. and stirred at RT for 1 h. After completion of starting material (acylation reaction, monitored by TLC), 2N aq. NaOH (9 mL) was added to the reaction mixture at 0° C. and stirred at 60° C. for 2 h. After completion of reaction (complete elimination, monitored by TLC and LCMS), the reaction mixture was diluted with water (10 mL) and extracted with 2-MeTHF (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X B C18 (19*250) 10μ] to get the title compound Compound 928 (275 mg, 35%).

¹H NMR (400 MHz, DMSO-d₆): δ 12.01 (s, 1H), 8.14-8.10 (m, 2H), 7.99 (d, J=2.4 Hz, 1H), 7.95 (s, 1H), 7.69 (d, J=8.8 Hz, 2H), 7.41 (dd, J=9.2 Hz, 2.8 Hz, 1H), 6.91 (brs, 1H), 6.86-6.83 (m, 3H), 6.26 (dd, J=17.2 Hz, 10 Hz, 1H), 6.10 (dd, J=17.2 Hz, 2 Hz, 1H), 5.59 (dd, J=10 Hz, 2 Hz, 1H), 4.09-4.06 (m, 1H), 3.95-3.92 (m, 1H), 3.86-3.84 (m, 4H), 3.79-3.77 (m, 1H), 3.75-3.72 (m, 4H), 2.95-2.89 (m, 1H), 2.75 (q, J=9.2 Hz, 1H), 1.89-1.86 (m, 1H), 1.78-1.75 (m, 1H), 1.56-1.42 (m, 2H).

LCMS=[M+H]⁺: 525.27; Purity=98.67%.

### Example 79

### Compound 929

### Preparation of 1-(4-(5-((4-(4-(3-oxa-8-azabicyclo[3.2.1]octan-8-yl)-7H-pyrrolo[2,3-d]pyrimidin-6-yl)phenyl)amino)pyrimidin-2-yl)piperazin-1-yl)prop-2-en-1-one (Compound 929)

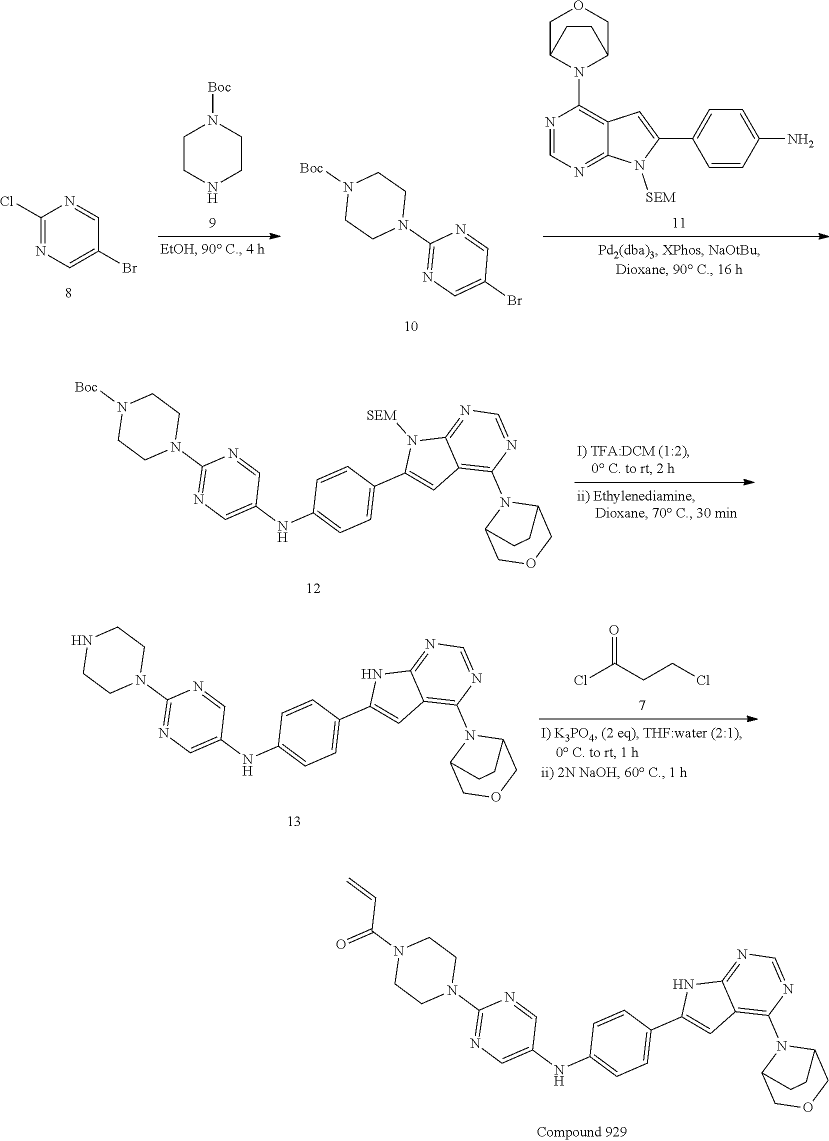

### Synthesis of tert-butyl 4-(5-bromopyrimidin-2-yl)piperazine-1-carboxylate (10)

To a stirred solution of 5-bromo-2-chloropyrimidine 1 (2 g, 8.41 mmol) in EtOH (15 mL) was added tert-butyl piperazine-1-carboxylate 9 (1.6 g, 8.41 mmol) at RT under N₂ atmosphere. The resulting reaction mixture was stirred at 90° C. for 4 h. After completion of reaction (monitored by TLC and LCMS), the reaction mixture was poured into water (50 mL) and extracted with EtOAc (3×100 mL). The combined organic layer was washed with ice cold brine (2×50 mL), dried over Na₂SO₄, filtered and concentrated under reduced pressure. The crude was purified by flash column chromatography (silica gel 100-200M, 40 g SNAP) using 8-10% EtOAc in heptane to get the title compound as a white solid 10 (2.3 g, 80%).

LCMS=[M+H]⁺: 343.06; Purity=95%.

### Synthesis of tert-butyl 4-(5-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)pyrimidin-2-yl)piperazine-1-carboxylate (12)

To a stirred solution of tert-butyl 4-(5-bromopyrimidin-2-yl)piperazine-1-carboxylate 10 (700 mg, 2.04 mmol) and 4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)aniline 11 (920 mg, 2.04 mmol) in 1,4-dioxane (15 mL) was added NaOtBu (300 mg, 3.06 mmol) at RT. The resulting reaction mixture was degassed with N₂ gas for 10 min, followed by XPhos (98 mg, 0.204 mmol) and Pd₂(dba)₃ (165 mg, 0.204 mmol) were added and stirred at 90° C. for 16 h. After completion of reaction (monitored by TLC and LCMS), the reaction mixture was filtered through celite pad and washed with EtOAc (100 mL). The filtrate was washed with water (2×50 mL). The organic layer was separated, dried over Na₂SO₄, filtered and evaporated under reduced pressure. The crude was purified by flash column chromatography (silica gel 100-200 M, 24 g SNAP) using 20-30% EtOAc in heptane to get the title compound as a light yellow solid 12 (1 g, 28%).

LCMS=[M+H]⁺: 714.41; Purity=68%.

### Synthesis of N-(4-(4-(3-oxa-8-azabicyclo[3.2.1]octan-8-yl)-7H-pyrrolo[2,3-d]pyrimidin-6-yl)phenyl)-2-(piperazin-1-yl)pyrimidin-5-amine (13)

To a stirred solution of tert-butyl 4-(5-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)pyrimidin-2-yl)piperazine-1-carboxylate 12 (1 g, 1.4 mmol) in DCM (20 mL) was added TFA (10 mL) dropwise under N₂ atmosphere at 0° C. The reaction mixture was stirred at RT for 2 h. After completion of reaction (monitored by TLC and LCMS), the reaction mixture was concentrated under reduced pressure, triturated with Et2O and dried under reduced pressure. The obtained crude was dissolved in 1,4-dioxane (10 mL) and ethylenediamine (0.3 mL, 4.2 mmol) was added to the reaction mixture and stirred at 70° C. for 30 min. After completion of reaction (monitored by LCMS), the reaction was poured into ice cold water (60 mL) and extracted with 25% IPA in CHCl₃ (3×50 mL). The combined organic layer was dried over Na₂SO₄, filtered and concentrated under reduced pressure to get the title compound as brown solid 15 (600 mg; crude) which was used in next step directly without further purification.

LCMS=[M+H]⁺: 484.23; Purity=60%.

### Synthesis of 1-(4-(5-((4-(4-(3-oxa-8-azabicyclo[3.2.1]octan-8-yl)-7H-pyrrolo[2,3-d]pyrimidin-6-yl)phenyl)amino)pyrimidin-2-yl)piperazin-1-yl)prop-2-en-1-one (Compound 929)

To an ice cold stirred solution of N-(4-(4-(3-oxa-8-azabicyclo[3.2.1]octan-8-yl)-7H-pyrrolo[2,3-d]pyrimidin-6-yl)phenyl)-2-(piperazin-1-yl)pyrimidin-5-amine 15 (600 mg, 1.24 mmol) in THF (10 mL) was added K₃PO₄ (530 mg, 2.48 mmol) dissolved in water (5 mL) under N₂ atmosphere and stirred for 5 min. Then added stock solution of 3-chloropropionyl chloride 7 (145 μL, 1.49 mmol) in THF (1 mL) drop wise to the reaction mixture at 0° C. and stirred at RT for 2 h. After completion of starting material (acylation reaction, monitored by TLC), 2N aq. NaOH (7.5 mL, 14.8 mmol) was added to the reaction mixture at 0° C. and stirred at 60° C. for 1 h. After completion of reaction (complete elimination, monitored by TLC and LCMS), the reaction mixture was diluted with water (50 mL) and extracted with 2-MeTHF (2×100 mL). The combined organic layer was dried over Na₂SO₄, filtered and concentrated under reduced pressure. The crude was purified by RP-HPLC [2-98% 0.1% TFA in water/ACN; Column: X Select Phenyl Hexyl (19*250) 5μ] to get the title Compound 929 (58 mg, 9%).

¹H NMR (400 MHz, DMSO-d₆): δ 12.78 (brs, 1H), 8.34 (s, 2H), 8.29 (s, 1H), 8.04 (brs, 1H), 7.75 (d, J=8.4 Hz, 2H), 7.71 (brs, 1H), 6.85 (q, J=10.4 Hz, 3H), 6.15 (dd, J=16.8 Hz, 2.4 Hz, 1H), 5.72 (dd, J=10.8 Hz, 2 Hz, 1H), 4.99 (brs, 1H), 3.77-3.51 (m, 12H), 2.10-2.06 (m, 4H).

LCMS=[M+H]⁺: 538.26; Purity=98.50%.

### Example 80

### Compound 930

### Preparation of (E)-4-(dimethylamino)-1-(4-(5-((4-(4-morpholino-7H-pyrrolo[2,3-d]pyrimidin-6-yl)phenyl)amino)pyrimidin-2-yl)piperazin-1-yl)but-2-en-1-one (Compound 930)

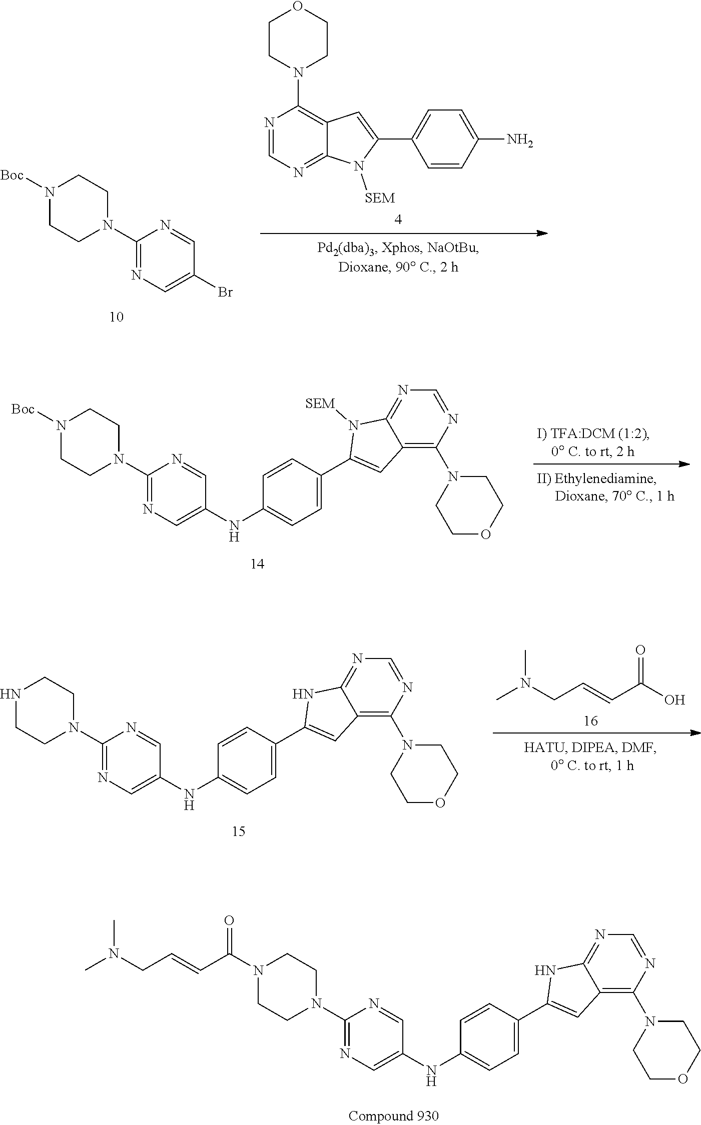

### Synthesis of tert-butyl 4-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperazine-1-carboxylate (14)

To a stirred solution of tert-butyl 4-(5-bromopyrimidin-2-yl)piperazine-1-carboxylate 10 (700 mg, 2.04 mmol) and 4-(4-morpholino-7-((2-(trimethylsilyl)ethoxy)methyl)-7H-pyrrolo[2,3-d]pyrimidin-6-yl)aniline 4 (920 mg, 2.04 mmol) in 1,4-dioxane (15 mL) was added NaOtBu (300 mg, 3.06 mmol). The resulting reaction mixture was degassed with N₂ gas for 10 min, followed by XPhos (98 mg, 0.204 mmol) and Pd₂(dba)₃ (165 mg, 0.204 mmol) were added to the reaction mixture. The reaction mixture was stirred at 90° C. for 2 h. After completion of reaction (monitored by TLC and LCMS), the reaction mixture was filtered through celite pad and washed with EtOAc (100 mL). The filtrate was washed with water (2×50 mL). The organic layer was separated, dried over Na₂SO₄, filtered and evaporated under reduced pressure. The crude was purified by flash column chromatography (silica gel 100-200 M, 12 g SNAP) using 20-30% EtOAc in heptane to get the title compound as a light yellow solid 14 (1 g, 28%).

LCMS=[M+H]⁺: 688.46; Purity=55%.

### Synthesis of N-(4-(4-morpholino-7H-pyrrolo[2,3-d]pyrimidin-6-yl)phenyl)-2-(piperazin-1-yl)pyrimidin-5-amine (15)

To a stirred solution of tert-butyl 4-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperazine-1-carboxylate 14 (1 g, 1.4 mmol) in DCM (20 mL) was added TFA (10 mL) dropwise under N₂ atmosphere at 0° C. and stirred at RT for 2 h. After completion of reaction (monitored by TLC and LCMS), the reaction mixture was concentrated under reduced pressure, triturated with Et2O and dried under reduced pressure. The crude was dissolved in 1,4-dioxane (10 mL) and ethylenediamine (0.3 mL, 4.2 mmol) was added to the reaction mixture and stirred at 70° C. for 1 h. After completion of reaction (monitored by LCMS), the reaction was poured into ice cold water (50 mL) and extracted with 25% IPA in CHCl₃ (3×60 mL). The combined organic layer was dried over Na₂SO₄, filtered and concentrated under reduced pressure to get the title compound as a brown solid 15 (600 mg; crude) which was used in next step directly without further purification.

LCMS=[M+H]⁺: 458.27; Purity=63%.

### Synthesis of (E)-4-(dimethylamino)-1-(4-(5-((4-(4-morpholino-7H-pyrrolo[2,3-d]pyrimidin-6-yl)phenyl)amino)pyrimidin-2-yl)piperazin-1-yl)but-2-en-1-one (Compound 930)

To a stirred solution of N-(4-(4-morpholino-7H-pyrrolo[2,3-d]pyrimidin-6-yl)phenyl)-2-(piperazin-1-yl)pyrimidin-5-amine 15 (500 mg, 1.09 mmol) and (E)-4-(dimethylamino)but-2-enoic acid 16 (215 mg, 1.64 mmol) in DMF (10 mL) were added DIPEA (0.95 mL, 4.46 mmol) and HATU (830 mg, 2.19 mmol) sequentially at 0° C. and stirred at RT for 1 h. After completion of reaction (monitored by TLC), the reaction mixture was poured into ice cold water (50 mL) and extracted with 25% IPA in CHCl₃ (3×50 mL). The combined organic layer was dried over Na₂SO₄, filtered and concentrated under reduced pressure. The crude was purified by RP-HPLC [30-70% 5 mM Ammonium Bicarbonate in Water/ACN; Column: XS PHENYL-HEXYL C8 (19*250) 5μ] to get the title compound Compound 930 (270 mg, 42%).

¹H NMR (400 MHz, DMSO-d₆): δ 12.48 (brs, 1H), 9.72 (brs, 1H), 8.35 (s, 2H), 8.24 (s, 1H), 8.02 (s, 1H), 7.73 (d, J=8.4 Hz, 2H), 7.07 (brs, 1H), 6.96 (d, J=14.8 Hz, 1H), 6.86-6.83 (m, 1H), 6.67-6.60 (m, 1H), 3.91-3.88 (m, 6H), 3.78-3.72 (m, 8H), 3.67-3.63 (m, 4H), 2.79-2.78 (m, 6H).

LCMS=[M+H]⁺: 569.20; Purity=96.23%.

### Example 81

### Compound 931

### Preparation of (E)-4-(azetidin-1-yl)-1-(4-(5-((4-(4-morpholino-7H-pyrrolo[2,3-d]pyrimidin-6-yl)phenyl)amino)pyrimidin-2-yl)piperazin-1-yl)but-2-en-1-one (Compound 931)

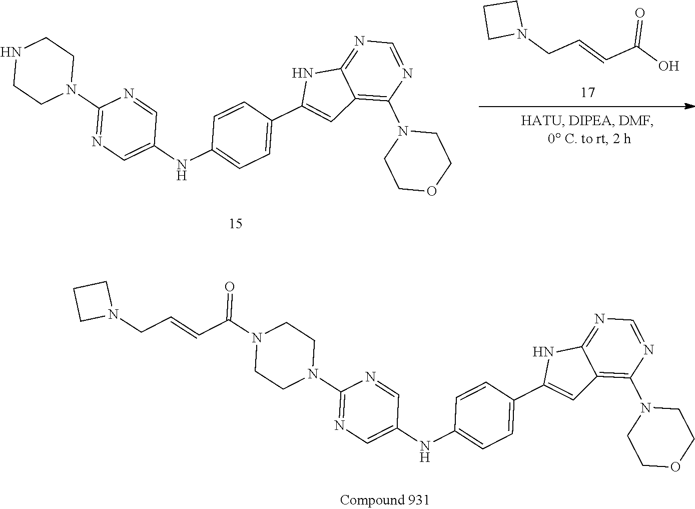 [SMILES: c1nc(N2CCOCC2)c2cc(-c3ccc(Nc4cnc(N5CCNCC5)nc4)cc3)[nH]c2n1 ; O=C(O)/C=C/CN1CCC1 ; O=C(/C=C/CN1CCC1)N1CCN(c2ncc(Nc3ccc(-c4cc5c(N6CCOCC6)ncnc5[nH]4)cc3)cn2)CC1]

To a stirred solution of N-(4-(4-morpholino-7H-pyrrolo[2,3-d]pyrimidin-6-yl)phenyl)-2-(piperazin-1-yl)pyrimidin-5-amine 15 (300 mg, 0.656 mmol) and (E)-4-(azctidin-1-yl)but-2-enoic acid 17 (111 mg, 0.787 mmol) in DMF (8 mL) were added DIPEA (0.35 mL, 1.97 mmol) and HATU (499 mg, 1.310 mmol) sequentially at 0° C. and stirred at RT for 2 h. After completion of reaction (monitored by TLC), the reaction mixture was poured into ice cold water (50 mL) and extracted with 25% IPA in CHCl₃ (3×50 mL). The combined organic layer was dried over Na₂SO₄, filtered and concentrated under reduced pressure. The crude was purified by RP-HPLC [5-95% 0.1% TFA in Water/ACN; Column: X Bridge C8 (19*250) 5μ] to get the title Compound 931 (16 mg, 4%).

¹H NMR (400 MHz, DMSO-d₆): δ 12.26 (brs, 1H), 9.90 (brs, 1H), 8.35 (s, 2H), 8.19 (s, 1H), 7.72 (d, J=8.4 Hz, 2H), 7.08 (brs, 1H), 6.95-6.83 (m, 3H), 6.54-6.47 (m, 1H), 4.18-4.13 (m, 2H), 4.06-3.96 (m, 4H), 3.88-3.82 (m, 4H), 3.75-3.67 (m, 8H), 3.64-3.56 (m, 4H).

LCMS=[M+H]⁺: 581.23; Purity=96.07%.

### Example 82

### Compound 932

### Preparation of 1-(4-(5-((4-(4-morpholino-7H-pyrrolo[2,3-d]pyrimidin-6-yl)phenyl)amino)pyridin-2-yl)piperazin-1-yl)prop-2-en-1-one (Compound 932)

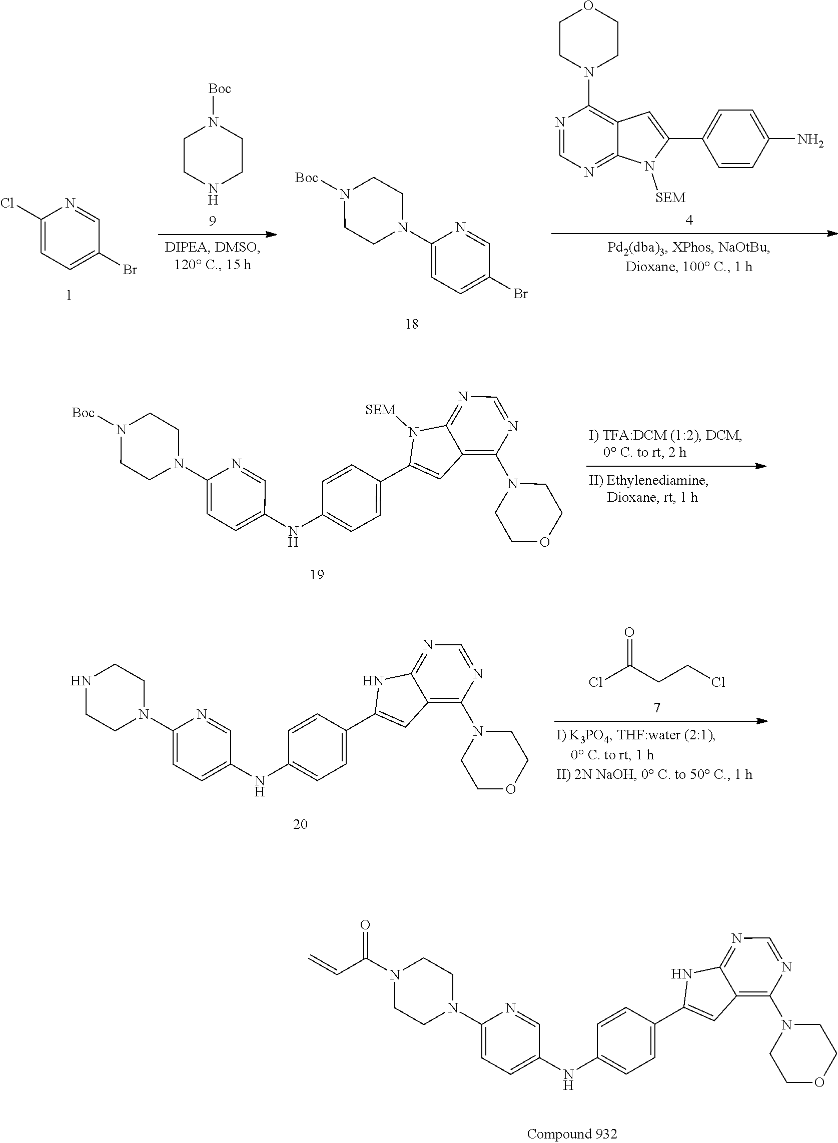

### Synthesis of tert-butyl 4-(5-bromopyridin-2-yl)piperazine-1-carboxylate (18)

To a stirred solution of 5-bromo-2-chloropyridine 1 (1 g, 5.2 mmol) and tert-butyl piperazine-1-carboxylate 9 (1.2 g, 6.24 mmol) in DMSO (15 mL) was added DIPEA (2.7 mL, 15.6 mmol) at RT under N₂ atmosphere. The resulting reaction mixture was stirred at 120° C. for 16 h. After completion of reaction (monitored by TLC and LCMS), the reaction mixture was poured into ice cold water (100 mL) and extracted with EtOAc (3×70 mL). The combined organic layer was washed with ice cold brine (2×60 mL), dried over Na2SO₄, filtered and concentrated under reduced pressure. The crude was purified by flash column chromatography (silica gel 100-200M, 12 g SNAP) using 8-10% EtOAc in heptane to get the title compound as a white solid 3 (600 mg, 34%).

¹H NMR (400 MHz, DMSO-d₆): δ 8.17 (d, J=2.4 Hz, 1H), 7.71-7.68 (m, 1H), 6.83 (d, J=9.2 Hz, 1H), 3.48-3.45 (m, 4H), 3.41-3.31 (m, 4H), 1.41 (s, 9H).

### Synthesis of tert-butyl 4-(5-((4-(4-morpholino-7-((2-(trimethylsilyl)ethoxy)methyl)-7H-pyrrolo[2,3-d]pyrimidin-6-yl)phenyl)amino)pyridin-2-yl)piperazine-1-carboxylate (19)

To a stirred solution of tert-butyl 4-(5-bromopyridin-2-yl)piperazine-1-carboxylate 18 (600 mg, 1.75 mmol) and 4-(4-morpholino-7-((2-(trimethylsilyl)ethoxy)methyl)-7H-pyrrolo[2,3-d]pyrimidin-6-yl)aniline 4 (746 mg, 1.75 mmol) in 1,4-dioxane (10 mL) was added NaOtBu (253 mg, 2.63 mmol). The resulting reaction mixture was degassed with N₂ gas for 10 min, followed by XPhos (84 mg, 0.175 mmol) and Pd₂(dba)₃ (142 mg, 0.175 mmol) were added to the reaction mixture at RT. The reaction mixture was stirred at 100° C. for 1 h. After completion of reaction (monitored by TLC and LCMS), the reaction mixture was filtered through celite pad and washed with EtOAc (100 mL). The filtrate was washed with water (2×50 mL). The organic layer was separated, dried over Na₂SO₄, filtered and evaporated under reduced pressure. The crude was purified by flash column chromatography (silica gel 100-200 M, 12 g SNAP) using 20-30% EtOAc in heptane to get the title compound as brown sticky solid 19 (300 mg, 25%).

LCMS=[M+H]⁺: 687.48; Purity=65%.

### Synthesis of N-(4-(4-morpholino-7H-pyrrolo[2,3-d]pyrimidin-6-yl)phenyl)-6-(piperazin-1-yl)pyridin-3-amine (20)

To a stirred solution of tert-butyl 4-(5-((4-(4-morpholino-7-((2-(trimethylsilyl)ethoxy)methyl)-7H-pyrrolo[2,3-d]pyrimidin-6-yl)phenyl)amino)pyridin-2-yl)piperazine-1-carboxylate 19 (300 mg, 0.437 mmol) in DCM (6 mL) was added TFA (3 mL) dropwise under N₂ atmosphere at 0° C. The reaction mixture was stirred at RT for 2 h. After completion of reaction (monitored by TLC and LCMS), the reaction mixture was concentrated under reduced pressure, triturated with Et2O and dried under reduced pressure. The crude was dissolved in 1,4-dioxane (5 mL) and ethylenediamine (44 μL, 0.655 mmol) was added to the reaction mixture and stirred at RT for 1 h. After completion of reaction (monitored by LCMS), the reaction was poured into ice cold water (50 mL) and extracted with 25% IPA in CHCl₃ (3×50 mL). The combined organic layer was dried over Na₂SO₄, filtered and concentrated under reduced pressure to get the title compound as brown solid 20 (180 mg; crude) which was used in next step directly without further purification.

LCMS=[M+H]⁺: 457.25; Purity=60%.

### Synthesis of 1-(4-(5-((4-(4-morpholino-7H-pyrrolo[2,3-d]pyrimidin-6-yl)phenyl)amino)pyridin-2-yl)piperazin-1-yl)prop-2-en-1-one (Compound 932)

To an ice cold stirred solution of N-(4-(4-morpholino-7H-pyrrolo[2,3-d]pyrimidin-6-yl)phenyl)-6-(piperazin-1-yl)pyridin-3-amine 20 (250 mg, 0.548 mmol) in THF (6 mL) was added K₃PO₄ (232 mg, 1.1 mmol) dissolved in water (3 mL) under N₂ atmosphere and stirred for 5 min. Then added stock solution of 3-chloropropionyl chloride 7 (63 μL, 0.657 mmol) in THF (1 mL) drop wise to the reaction mixture at 0° C. and stirred at RT for 1 h. After completion of starting material (acylation reaction, monitored by TLC), 2N aq. NaOH (3 mL, 6.57 mmol) was added to the reaction mixture at 0° C. and stirred at 50° C. for 1 h. After completion of reaction (complete elimination, monitored by TLC and LCMS), the reaction mixture was diluted with water (10 mL) and extracted with 2-MeTHF (3×50 mL). The combined organic layer was dried over Na₂SO₄, filtered and concentrated under reduced pressure. The crude was purified by RP-HPLC [10-95% 0.1% TFA in Water/ACN; Column: X Select Fluoro Phenyl (19*250) 5μ] to get the title Compound 932 (65 mg, 23%).

¹H NMR (400 MHz, DMSO-d₆): δ 12.65 (brs, 1H), 8.28 (s, 1H), 8.16 (brs, 1H), 8.00 (d, J=2.4 Hz, 1H), 7.74 (d, J=8.4 Hz, 2H), 7.54 (d, J=8.8 Hz, 1H), 7.14 (s, 1H), 6.98 (d, J=8.8 Hz, 1H), 6.92-6.83 (m, 3H), 6.15 (dd, J=16.4 Hz, 2.4 Hz, 1H), 5.72 (dd, J=10.4 Hz, 2.4 Hz, 1H), 3.93-3.91 (m, 4H), 3.80-3.78 (m, 4H), 3.71-3.67 (m, 4H), 3.52-3.48 (m, 4H).

LCMS=[M+H]⁺: 511.22; Purity=95.26%.

### Example 83

### Compound 933

### Preparation of (R)—N-(1-(5-((4-(4-morpholino-7H-pyrrolo[2,3-d]pyrimidin-6-yl)phenyl)amino)pyrazin-2-yl)piperidin-3-yl)acrylamide (Compound 933)

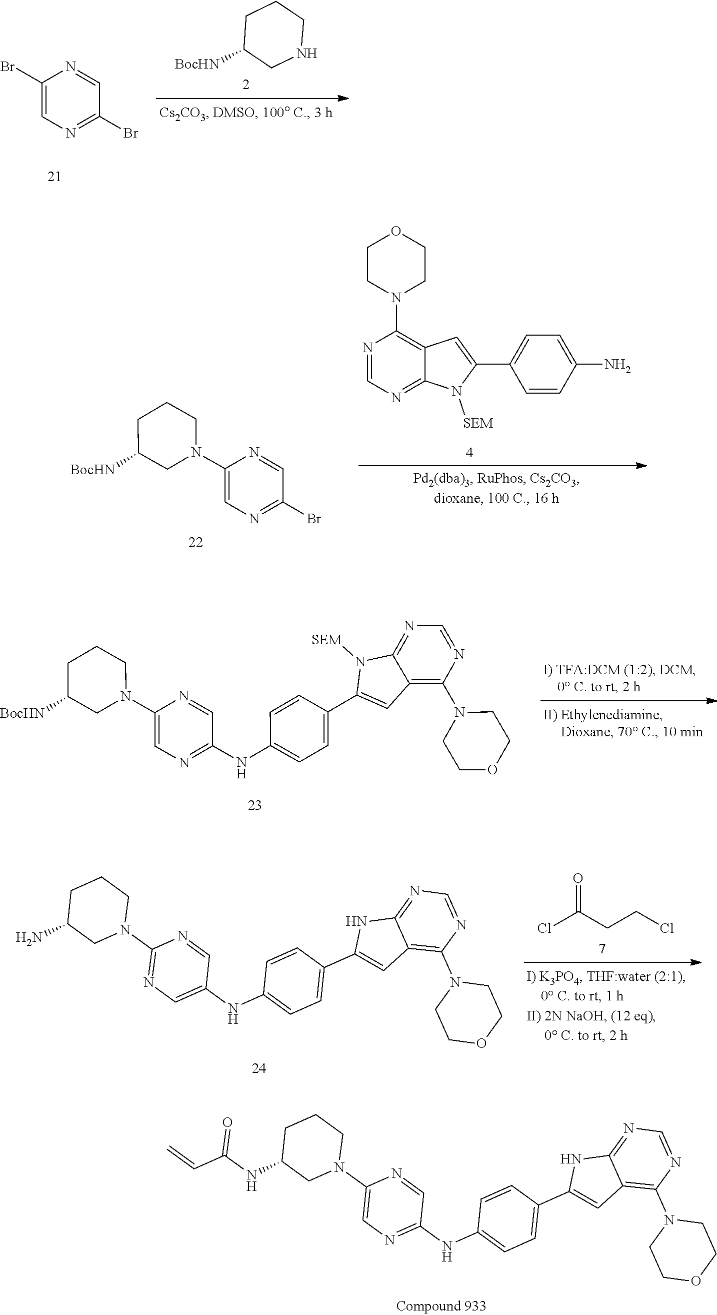

### Synthesis of tert-butyl (R)-(1-(5-bromopyrazin-2-yl)piperidin-3-yl)carbamate (22)

To a stirred solution of 2,5-dibromopyrazine 21 (2 g, 8.41 mmol) and tert-butyl (R)-piperidin-3-ylcarbamate 2 (1.68 g, 8.41 mmol) in DMSO (20 mL) was added Cs₂CO₃ (4.11 g, 12.6 mmol) at RT under N₂ atmosphere. The resulting reaction mixture was stirred at 100° C. for 3 h. After completion of reaction (monitored by TLC and LCMS), the reaction mixture was poured into ice cold water (100 mL) and extracted with EtOAc (3×100 mL). The combined organic layer was washed with ice cold brine (2×100 mL), dried over Na₂SO₄, filtered and concentrated under reduced pressure. The crude was purified by flash column chromatography (silica gel 100-200M, 12 g SNAP) using 8-10% EtOAc in heptane to get the desired compound as an off white solid 22 (2.5 g, 83%).

LCMS=[M+H]⁺: 357.23; Purity=89%.

### Synthesis of tert-butyl (R)-(1-(5-((4-(4-morpholino-7-((2-(trimethylsilyl)ethoxy)methyl)-7H-pyrrolo[2,3-d]pyrimidin-6-yl)phenyl)amino)pyrazin-2-yl)piperidin-3-yl)carbamate (23)

To a stirred solution of tert-butyl (R)-(1-(5-bromopyrazin-2-yl)piperidin-3-yl)carbamate 22 (2.5 g, 7 mmol) and 4-(4-morpholino-7-((2-(trimethylsilyl)ethoxy)methyl)-7H-pyrrolo[2,3-d]pyrimidin-6-yl)aniline 4 (2.98 g, 7 mmol) in 1,4-dioxane (15 mL) was added Cs₂CO₃ (6.84 g, 21 mmol). The resulting reaction mixture was degassed with N₂ gas for 10 min, followed by RuPhos (653 mg, 1.4 mmol) and Pd₂(dba)₃ (566 mg, 0.7 mmol) were added to the reaction mixture. The reaction mixture was stirred at 100° C. for 16 h. After completion of reaction (monitored by TLC and LCMS), the reaction mixture was filtered through celite pad and washed with EtOAc (100 mL). The filtrate was washed with water (2×50 mL). The organic layer was separated, dried over Na₂SO₄, filtered and evaporated under reduced pressure. The crude was purified by flash column chromatography (silica gel 100-200 M, 12 g SNAP) using 20-30% EtOAc in heptane to get the title compound as a brown sticky solid 23 (2.5 g, 51%).

LCMS=[M+H]⁺: 702.48; Purity=32%.

### Synthesis of (R)-5-(3-aminopiperidin-1-yl)-N-(4-(4-morpholino-7H-pyrrolo[2,3-d]pyrimidin-6-yl)phenyl)pyrazin-2-amine (24)

To a stirred solution of tert-butyl (R)-(1-(5-((4-(4-morpholino-7-((2-(trimethylsilyl)ethoxy)methyl)-7H-pyrrolo[2,3-d]pyrimidin-6-yl)phenyl)amino)pyrazin-2-yl)piperidin-3-yl)carbamate 23 (2.5 g 3.56 mmol) in DCM (20 mL) was added TFA (10 mL) dropwise under N₂ atmosphere at 0° C. The reaction mixture was stirred at RT for 2 h. After completion of reaction (monitored by TLC and LCMS), the reaction mixture was concentrated under reduced pressure, triturated with Et2O and dried under reduced pressure. The obtained crude was dissolved in 1,4-dioxane (20 mL) and ethylenediamine (0.4 mL, 5.34 mmol) was added to the reaction mixture and stirred at 70° C. for 10 min. After completion of reaction (monitored by LCMS), the reaction was poured into ice cold water (100 mL) and extracted with 25% IPA in CHCl₃ (3×100 mL). The combined organic layer was dried over Na₂SO₄, filtered and concentrated under reduced pressure to get the title compound as a yellow solid 24 (1.5 g: crude) which was used in next step directly without further purification.

LCMS=[M+H]⁺: 472.31

### Synthesis of (R)—N-(1-(5-((4-(4-morpholino-7H-pyrrolo[2,3-d]pyrimidin-6-yl)phenyl)amino)pyrazin-2-yl)piperidin-3-yl)acrylamide (Compound 933)

To an ice cold stirred solution of (R)-5-(3-aminopiperidin-1-yl)-N-(4-(4-morpholino-7H-pyrrolo[2,3-d]pyrimidin-6-yl)phenyl)pyrazin-2-amine 24 (1.5 g, 3.18 mmol) in THF (14 mL) was added K₃PO₄ (1.35 g, 6.36 mmol) dissolved in water (7 mL) under N₂ atmosphere and stirred for 5 min. Then added stock solution of 3-chloropropionyl chloride 7 (0.4 mL, 3.82 mmol) in THF (1 mL) drop wise to the reaction mixture at 0° C. and stirred at RT for 1 h. After completion of starting material (acylation reaction, monitored by TLC), 2N aq. NaOH (19 mL) was added to the reaction mixture at 0° C. and stirred at RT for 2 h. After completion of reaction (complete elimination, monitored by TLC and LCMS), the reaction mixture was diluted with water (100 mL) and extracted with 2-MeTHF (2×100 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select CSH C18 (19*250) 10μ] to get the title Compound 933 (44 mg, 2.6%).

¹H NMR (400 MHz, DMSO-d₆): δ 12.07 (brs, 1H), 9.07 (s, 1H), 8.15-8.13 (m, 2H), 7.94 (d, J=5.6 Hz, 2H), 7.77 (d, J=8.8 Hz, 2H), 7.56 (d, J=8.8 Hz, 2H), 6.98 (brs, 1H), 6.26 (dd, J=17.2 Hz, 10 Hz, 1H), 6.11 (dd, J=17.2 Hz, 2 Hz, 1H), 5.60 (dd, J=10 Hz, 2 Hz, 1H), 4.01-3.98 (m, 1H), 3.86-3.82 (m, 6H), 3.75-3.74 (m, 4H), 2.92 (t, J=10 Hz, 1H), 2.77-2.74 (m, 1H), 1.88-1.85 (m, 1H), 1.80-1.76 (m, 1H), 1.61-1.42 (m, 2H).

LCMS=[M+H]⁺: 526.25; Purity=98.17%.

### Example 84

### Compound 934

### Preparation of (E)-N-(4-(4-morpholino-7H-pyrrolo[2,3-d]pyrimidin-6-yl)phenyl)-4-((3-(prop-1-en-1-ylsulfonyl)piperidin-1-yl)methyl)picolinamide (Compound 934)

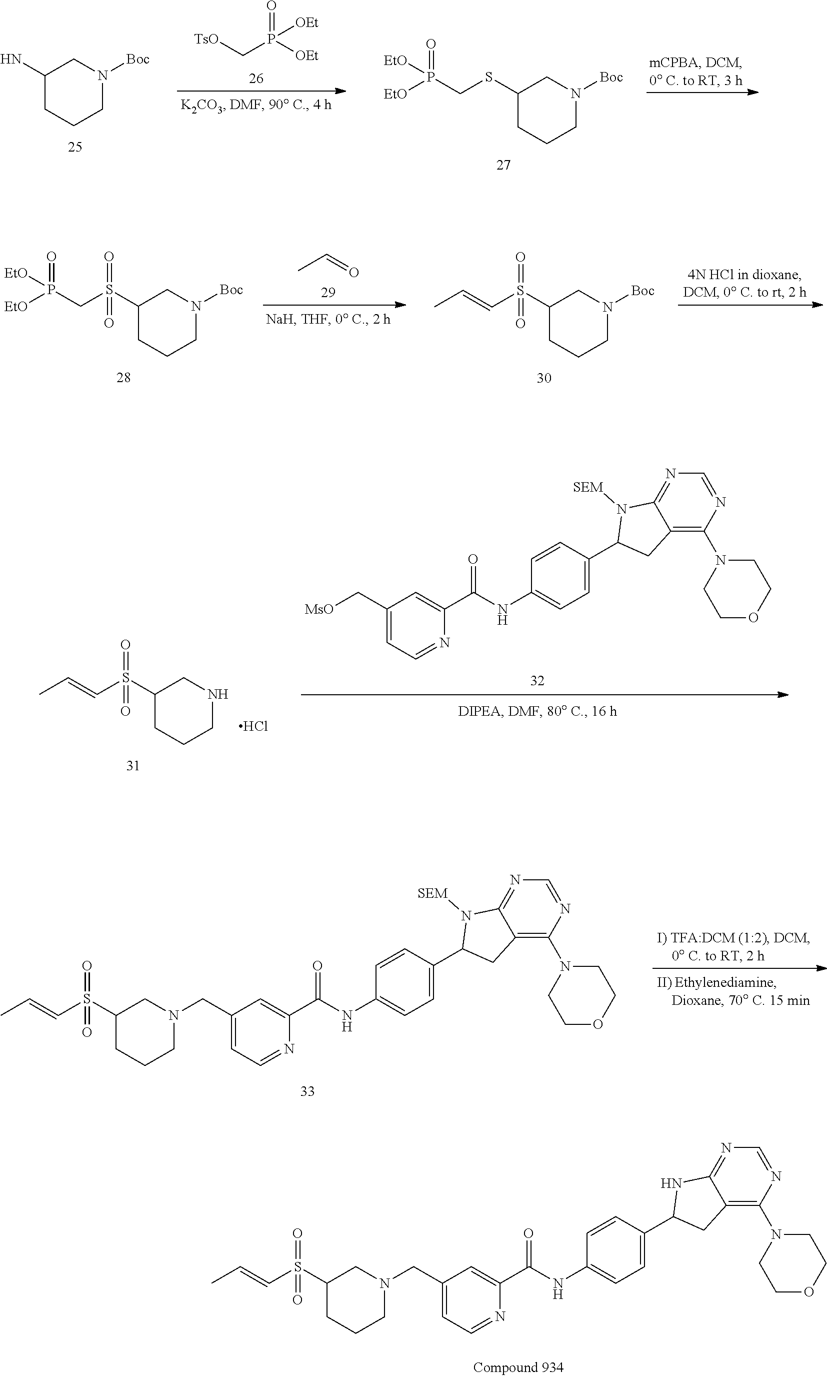

### Synthesis of tert-butyl 3-(((diethoxyphosphoryl)methyl)thio)piperidine-1-carboxylate (27)

To a stirred solution of tert-butyl 3-mercaptopiperidine-1-carboxylate 25 (1 g, 4.6 mmol) in DMF (10 mL) was added K₂CO₃ (1.9 g, 13.8 mmol) at RT and stirred for 5 min under N₂ atmosphere, followed by (diethoxyphosphoryl)methyl 4-methylbenzenesulfonate 26 (1.78 g, 5.52 mmol) was added to the reaction mixture. The resulting reaction mixture was stirred at 90° C. for 4 h. After completion of reaction (monitored by TLC), the reaction mixture was cooled to RT and poured into ice cold-water (100 mL) and extracted with EtOAc (3×100 mL). The combined organic layer was washed with ice cold brine (2×100 mL), dried over Na2SO₄, filtered and evaporated under reduced pressure. The crude was purified by flash column chromatography (silica gel 100-200 M, 40 g SNAP) using 2-3% MeOH in DCM to get the title compound as colourless liquid 27 (1.5 g, 89%).

LCMS=[M+H]⁺: 368.91; Purity=65%.

### Synthesis of tert-butyl 3-(((diethoxyphosphoryl)methyl) sulfonyl)piperidine-1-carboxylate (28)

To a stirred solution of tert-butyl 3-(((diethoxyphosphoryl)methyl)thio)piperidine-1-carboxylate 27 (1.5 g, 4.08 mmol) in DCM (20 mL) was added m-CPBA (1.9 g, 8.16 mmol; ≤77% purity) at 0° C. under N₂ atmosphere. The reaction mixture was stirred at RT for 3 h. After completion of reaction (monitored by TLC), the reaction mixture was quenched with saturated NaHCO₃ and extracted with 25% IPA in CHCl₃ (3×50 mL). The combined organic layer was washed with brine (2×50 mL), dried over Na₂SO₄, filtered and dried under reduced pressure. The crude was purified by flash column chromatography (silica gel 100-200 M, 40 g SNAP) using 4-5% MeOH: DCM to get the title compound as colourless liquid 28 (1 g, 61%).

LCMS=[M−H]: 398.24; Purity=60%.

### Synthesis of tert-butyl (E)-3-(prop-1-en-1-ylsulfonyl)piperidine-1-carboxylate (30)

To a stirred solution of tert-butyl 3-(((diethoxyphosphoryl)methyl) sulfonyl)piperidine-1-carboxylate 28 (1 g, 2.5 mmol) in THF (10 mL) was added NaH (150 mg, 3.76 mmol; 60% dispersion in mineral oil) at 0° C. under N₂ atmosphere and stirred for 30 min. Then stock solution of acetaldehyde (560 μL, 10 mmol) in

THF (2 mL) was added dropwise to the reaction mixture and stirred at 0° C. for 2 h. After completion of reaction (monitored by TLC), the reaction mixture was quenched with ice cold-water (50 mL) and extracted with EtOAc (3×50 mL). The combined organic layer was washed with brine (2×50 mL), dried over Na₂SO₄, filtered and evaporated under reduced pressure. The crude was purified by flash column chromatography (silica gel 100-200 M, 24 g SNAP) using 3-4% MeOH in DCM eluent to get the title compound as colorless liquid 30 (450 mg, 62%).

LCMS=[M+H]⁺: 290.16; Purity=85%.

### Synthesis of (E)-3-(prop-1-en-1-ylsulfonyl)piperidine hydrochloride (31)

To an ice cold stirred solution of tert-butyl (E)-3-(prop-1-en-1-ylsulfonyl)piperidine-1-carboxylate 30 (450 mg, 1.55 mmol) in DCM (5 mL) was added 4N HCl in 1,4-dioxane (4 mL, 15.5 mmol) dropwise under N₂ atmosphere. The resulting reaction mixture was stirred at RT for 2 h. After completion of reaction (monitored by TLC and LCMS), the reaction mixture was evaporated under reduced pressure to get the title compound as white sticky solid 31 (300 mg; crude) which was used in next step without further purification.

### Synthesis of (E)-N-(4-(4-morpholino-7-((2-(trimethylsilyl)ethoxy)methyl)-7H-pyrrolo[2,3-d]pyrimidin-6-yl)phenyl)-4-((3-(prop-1-en-1-ylsulfonyl)piperidin-1-yl)methyl)picolinamide (33)

To a stirred solution of (E)-3-(prop-1-en-1-ylsulfonyl)piperidine hydrochloride 28 (100 mg, 0.443 mmol) and (2-((4-(4-morpholino-7-((2-(trimethylsilyl)ethoxy)methyl)-7H-pyrrolo[2,3-d]pyrimidin-6-yl)phenyl)carbamoyl)pyridin-4-yl)methyl methanesulfonate 32 (285 mg, 0.443 mmol) in DMF (8 mL) was added DIPEA (0.25 mL, 1.33 mmol) at RT under N₂ atmosphere. The reaction mixture was stirred at 80° C. for 16 h. After completion of reaction (monitored by TLC), the reaction mixture was diluted with water (50 mL) and extracted with EtOAc (3×50 mL). The combined organic layer was washed with ice cold brine (2×50 mL), dried over Na₂SO₄, filtered and evaporated under reduced pressure to get the title compound as brown sticky solid 33 (280 mg; crude) which was used in next step without further purification.

LCMS=[M+H]⁺: 732.43; Purity=60%.

### Synthesis of (E)-N-(4-(4-morpholino-7H-pyrrolo[2,3-d]pyrimidin-6-yl)phenyl)-4-((3-(prop-1-en-1-ylsulfonyl)piperidin-1-yl)methyl)picolinamide (Compound 934)

To an ice cold stirred solution of (E)-N-(4-(4-morpholino-7-((2-(trimethylsilyl)ethoxy)methyl)-7H-pyrrolo[2,3-d]pyrimidin-6-yl)phenyl)-4-((3-(prop-1-en-1-ylsulfonyl)piperidin-1-yl)methyl)picolinamide 33 (280 mg, 0.383 mmol) in DCM (5 mL) was added TFA (2.5 mL) dropwise at 0° C. under N₂ atmosphere. The reaction mixture was stirred at RT for 2 h. After completion of reaction (monitored by TLC), the reaction mixture was concentrated under reduced pressure. The obtained crude was dissolved in 1,4-dioxane (5 mL) and ethylenediamine (40 μL, 0.575 mmol) was added to the reaction mixture and stirred at 70° C. for 15 min. After completion of reaction (monitored by LCMS), the reaction mixture was diluted with water (30 mL) and extracted with 25% IPA in CHCl₃ (3×30 mL). The combined organic layer was dried over Na₂SO₄, filtered and concentrate under reduced pressure. The crude was purified by RP-HPLC [2-98% 5 mM Ammonium Bicarbonate in Water/ACN; Column: X Select CSH C18 (19*250) 10μ] to get the title compound Compound 934 (9 mg, 4%).

¹H NMR (400 MHz, DMSO-d₆): δ 12.19 (s, 1H), 10.74 (s, 1H), 8.69 (d, J=4.8 Hz, 1H), 8.18 (s, 1H), 8.11 (s, 1H), 8.00-7.98 (m, 2H), 7.92-7.90 (m, 2H), 7.61 (d, J=4.4 Hz, 1H), 7.16 (s, 1H), 6.77-6.69 (m, 1H), 6.63-6.59 (m, 1H), 3.93-3.87 (m, 4H), 3.76-3.67 (m, 6H), 3.20-3.14 (m, 1H), 3.10-3.07 (m, 1H), 2.77-2.74 (m, 1H), 2.13 (t, J=10.8 Hz, 1H), 2.02-1.97 (m, 2H), 1.91 (d, J=6.4 Hz, 3H), 1.77-1.74 (m, 1H), 1.58-1.49 (m, 1H), 1.41-1.33 (m, 1H).

LCMS=[M+H]⁺: 602.26; Purity=99.04%.

### Synthesis of (2-((4-(4-morpholino-7-((2-(trimethylsilyl)ethoxy)methyl)-7H-pyrrolo[2,3-d]pyrimidin-6-yl)phenyl)carbamoyl)pyridin-4-yl)methyl methanesulfonate (32)

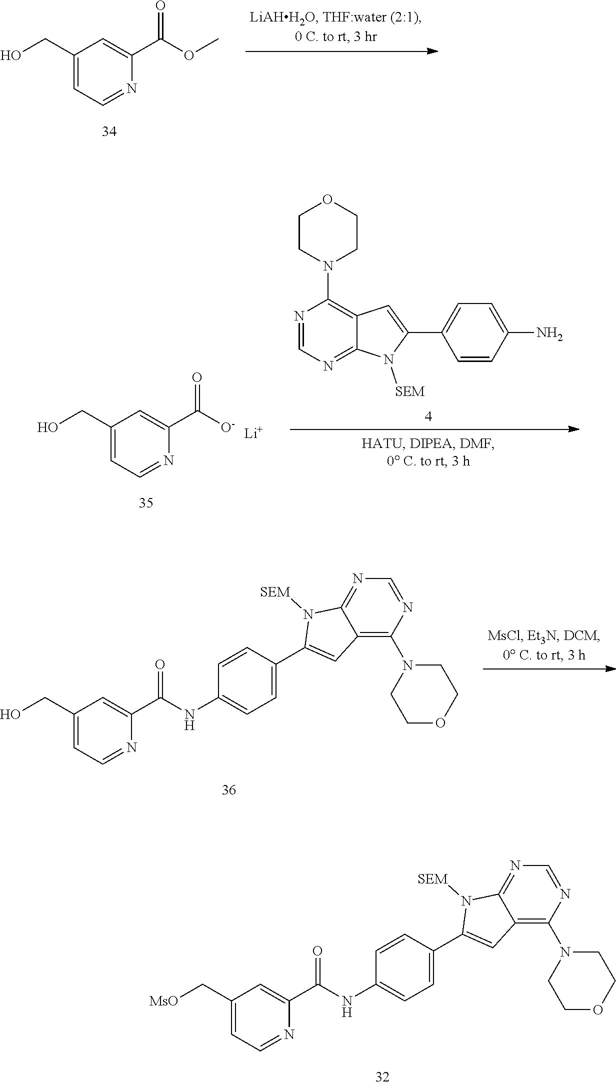 [SMILES: COC(=O)c1cc(CO)ccn1 ; O=C([O-])c1cc(CO)ccn1 ; [Li+] ; C[Si](C)(C)CCOCn1c(-c2ccc(N)cc2)cc2c(N3CCOCC3)ncnc21 ; C[Si](C)(C)CCOCn1c(-c2ccc(NC(=O)c3cc(CO)ccn3)cc2)cc2c(N3CCOCC3)ncnc21 ; C[Si](C)(C)CCOCn1c(-c2ccc(NC(=O)c3cc(COS(C)(=O)=O)ccn3)cc2)cc2c(N3CCOCC3)ncnc21]

### Synthesis of lithium 4-(hydroxymethyl)picolinate (35)

To a stirred solution of methyl 4-(hydroxymethyl)picolinate 34 (1 g, 5.98 mmol) in THF (10 mL) was added LiOH·H₂O (3 g, 71.8 mmol) dissolved in H₂O (5 mL) dropwise at 0° C. and stirred at RT for 3 h. After completion of reaction (monitored by TLC and LCMS), the reaction mixture was concentrated under reduced pressure. The crude was dissolved in 25% IPA in CHCl₃ (100 mL), stirred for 10 min at RT and filtered through sintered funnel. The filtrate was concentrated under reduced pressure to get the title compound as a white solid 35 (900 mg; crude) which was used in next step directly without further purification.

### Synthesis of 4-(4-morpholino-7-((2-(trimethylsilyl)ethoxy)methyl)-7H-pyrrolo[2,3-d]pyrimidin-6-yl)aniline (36)

To a stirred solution of lithium 4-(hydroxymethyl)picolinate 35 (900 mg, 5.66 mmol) and 4-(4-morpholino-7-((2-(trimethylsilyl)ethoxy)methyl)-7H-pyrrolo[2,3-d]pyrimidin-6-yl)aniline 4 (1.93 g, 4.53 mmol) in DMF (15 mL) were added DIPEA (2.3 mL, 13.59 mmol) and HATU (2.6 g, 6.79 mmol) sequentially at 0° C. The reaction mixture was stirred at RT for 3 h. After completion of reaction (monitored by TLC), the reaction mixture was poured into ice cold water (100 mL) and extracted with EtOAc (3×70 mL). The combined organic layer over dried over Na₂SO₄, filtered and concentrated under reduced pressure. The crude was purified by flash column chromatography (silica gel 100-200 M, 24 g SNAP) using 30-50% EtOAc in heptane to get the title compound as a brown solid 36 (900 mg, 30%).

LCMS=[M+H]⁺: 561.28; Purity=75%.

### Synthesis of (2-((4-(4-morpholino-7-((2-(trimethylsilyl)ethoxy)methyl)-7H-pyrrolo[2,3-d]pyrimidin-6-yl)phenyl)carbamoyl)pyridin-4-yl)methyl methanesulfonate (32)

To an ice cold stirred solution of 4-(hydroxymethyl)-N-(4-(4-morpholino-7-((2-(trimethylsilyl)ethoxy)methyl)-7H-pyrrolo[2,3-d]pyrimidin-6-yl)phenyl)picolinamide 36 (300 mg, 0.535 mmol) in DCM (6 mL) was added Et₃N (0.25 mL, 1.61 mmol) under N₂ atmosphere and stirred for 5 min, followed by MsCl (0.82 mL, 1.07 mmol) was added at 0° C. and stirred at RT for 3 h. After completion of reaction (monitored by TLC and LCMS), the reaction mixture was quenched with sat. NaHCO₃ (50 mL) and extracted with DCM (3×50 mL). The combined organic layer was dried over Na₂SO₄, filtered and evaporated under reduced pressure to get the title compound as brown sticky oil 32 (350 mg; crude) which was used in next step directly without further purification.

LCMS=[M+H]⁺: 639.23; Purity=50%.

### Example 85

### Compound 935

### Preparation of 1-(4-(5-((4-(4-morpholino-7H-pyrrolo[2,3-d]pyrimidin-6-yl)phenyl)amino)pyrazin-2-yl)piperazin-1-yl)prop-2-en-1-one (Compound 935)

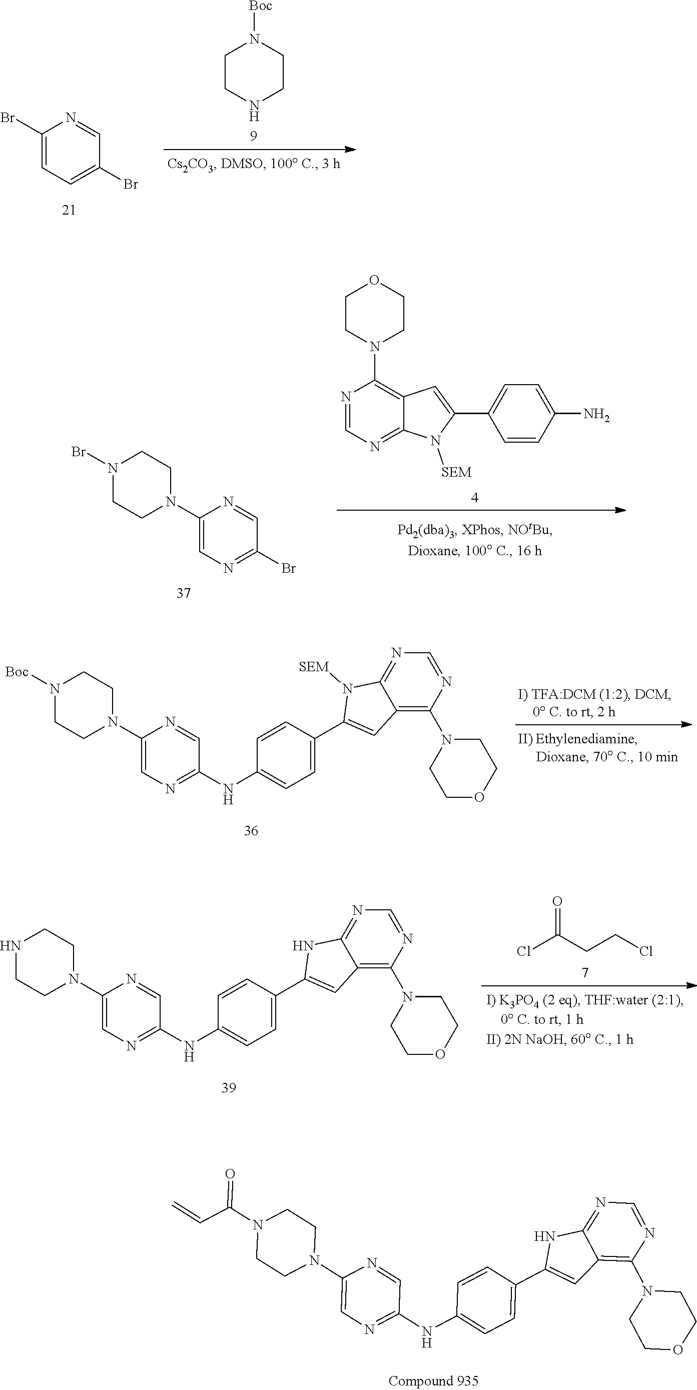

### Synthesis of tert-butyl 4-(5-bromo-2-pyrazinyl)-1-piperazinecarboxylate (37)

To a stirred solution of 2,5-dibromopyrazine 21 (2 g, 8.41 mmol) and tert-butyl piperazine-1-carboxylate 9 (1.57 g, 8.41 mmol) in DMSO (20 mL) was added Cs₂CO₃ (4.1 g, 12.6 mmol) at RT under N₂ atmosphere. The resulting reaction mixture was stirred at 100° C. for 3 h. After completion of reaction (monitored by TLC and LCMS), the reaction mixture was poured into ice cold water (100 mL) and extracted with EtOAc (3×100 mL). The combined organic layer was washed with ice cold brine (2×50 mL), dried over Na₂SO₄, filtered and concentrated under reduced pressure to get the title compound as a light yellow solid 37 (2.3 g, 79%) which was used in next step directly without purification.

¹H NMR (400 MHz, DMSO-d₆): δ 8.24 (s, 1H), 8.14 (s, 1H), 3.55-3.53 (m, 4H), 3.43-3.42 (m, 4H), 1.41 (s, 9H).

### Synthesis of tert-butyl 4-{5-[5-(4-morpholino-1-{[2-(trimethylsilyl) ethoxy]methyl}-1H-1,5,7-triazainden-2-yl)-2-pyrazinylamino]-2-pyrazinyl}-1-piperazinecarboxylate (38)

To a stirred solution of tert-butyl 4-(5-bromopyrazin-2-yl)piperazine-1-carboxylate 37 (1 g, 2.91 mmol) and 4-(4-morpholino-7-((2-(trimethylsilyl)ethoxy)methyl)-7H-pyrrolo[2,3-d]pyrimidin-6-yl)aniline 4 (1.24 g, 2.91 mmol) in 1,4-dioxane (15 mL) was added NaOtBu (420 mg, 4.37 mmol) at RT. The resulting reaction mixture was degassed with N₂ gas for 10 min, followed by Pd₂(dba)₃ (236 mg, 0.291 mmol) and XPhos (278 mg, 0.583 mmol) were added and stirred at 100° C. for 16 h. After completion of reaction (monitored by TLC and LCMS), the reaction mixture was filtered through Celite pad and washed with EtOAc (100 mL). The filtrate was washed with water (2×50 mL). The organic layer was separated, dried over Na₂SO₄, filtered and evaporated under reduced pressure. The crude was purified by flash column chromatography (silica gel 100-200 M, 12 g SNAP) using 20-30% EtOAc in heptane to get the title compound as a light yellow solid 38 (600 mg, 30%).

LCMS=[M+H]⁺: 688.48; Purity=75%.

### Synthesis of N-(4-(4-morpholino-7H-pyrrolo[2,3-d]pyrimidin-6-yl)phenyl)-5-(piperazin-1-yl)pyrazin-2-amine (39)

To a stirred solution of tert-butyl 4-(5-((4-(4-morpholino-7-((2-(trimethylsilyl)ethoxy)methyl)-7H-pyrrolo[2,3-d]pyrimidin-6-yl)phenyl)amino)pyrazin-2-yl)piperazine-1-carboxylate 38 (600 mg, 0.872 mmol) in DCM (8 mL) was added TFA (4 mL) dropwise under N₂ atmosphere at 0° C. The reaction mixture was stirred at RT for 2 h. After completion of reaction (monitored by TLC and LCMS), the reaction mixture was concentrated under reduced pressure, triturated with Et2O and dried under reduced pressure. The obtained crude was dissolved in 1,4-dioxane (10 mL) and ethylenediamine (0.1 mL, 1.31 mmol) was added to the reaction mixture and stirred at 70° C. for 10 min. After completion of reaction (monitored by LCMS), the reaction was poured into ice cold water (50 mL) and extracted with 25% IPA in CHCl₃ (3×50 mL). The combined organic layer was dried over Na₂SO₄, filtered and concentrated under reduced pressure to get the title compound as a brown solid 39 (300 mg; crude) which was used in next step directly without further purification.

LCMS=[M+H]⁺: 458.27; Purity=60%.

### Synthesis of 1-(4-(5-((4-(4-morpholino-7H-pyrrolo[2,3-d]pyrimidin-6-yl)phenyl)amino)pyrazin-2-yl)piperazin-1-yl)prop-2-en-1-one (Compound 935)

To an ice cold stirred solution of N-(4-(4-morpholino-7H-pyrrolo[2,3-d]pyrimidin-6-yl)phenyl)-5-(piperazin-1-yl)pyrazin-2-amine 39 (300 mg, 0.656 mmol) in THF (6 mL) was added K₃PO₄ (278 mg, 1.31 mmol) dissolved in water (3 mL) under N₂ atmosphere and stirred for 5 min. Then added stock solution of 3-chloropropionyl chloride 7 (93 μL, 0.787 mmol) in THF (1 mL) drop wise to the reaction mixture at 0° C. and stirred at RT for 1 h. After completion of starting material (acylation reaction, monitored by TLC), 2N aq. NaOH (4 mL, 7.87 mmol) was added to the reaction mixture at 0° C. and stirred at 60° C. for 1 h. After completion of reaction (complete elimination, monitored by TLC and LCMS), the reaction mixture was diluted with water (20 mL) and extracted with 2-Me THF (3×50 mL). The combined organic layer was dried over Na₂SO₄, filtered and concentrated under reduced pressure. The crude was purified by RP-HPLC [10-90% 0.1% TFA in Water/ACN; Column: X Select Fluoro Phenyl (19*250) 5μ] to get the title compound Compound 935 (47 mg, 10%).

¹H NMR (400 MHz, DMSO-d₆): δ 12.07 (s, 1H), 9.12 (s, 1H), 8.15 (s, 1H), 7.96 (d, J=4.4 Hz, 2H), 7.77 (d, J=8.4 Hz, 2H), 7.58 (d, J=8.8 Hz, 2H), 6.98 (s, 1H), 6.86 (dd, J=16.4 Hz, 10.4 Hz, 1H), 6.14 (dd, J=16.4 Hz, 2.4 Hz, 1H), 5.71 (dd, J=10.4 Hz, 2.4 Hz, 1H), 3.87-3.85 (m, 4H), 3.75-3.73 (m, 4H), 3.70-3.67 (m, 4H), 3.45-3.38 (m, 4H).

LCMS=[M+H]⁺: 512.24; Purity=98.17%.

### Example 86

### Compound 936

### Preparation of 1-(4-(3-(2,2,2-trifluoro-1-((4-(4-morpholino-7H-pyrrolo[2,3-d]pyrimidin-6-yl)phenyl)amino)ethyl)azetidin-1-yl)piperidin-1-yl)prop-2-en-1-one (Compound 936)

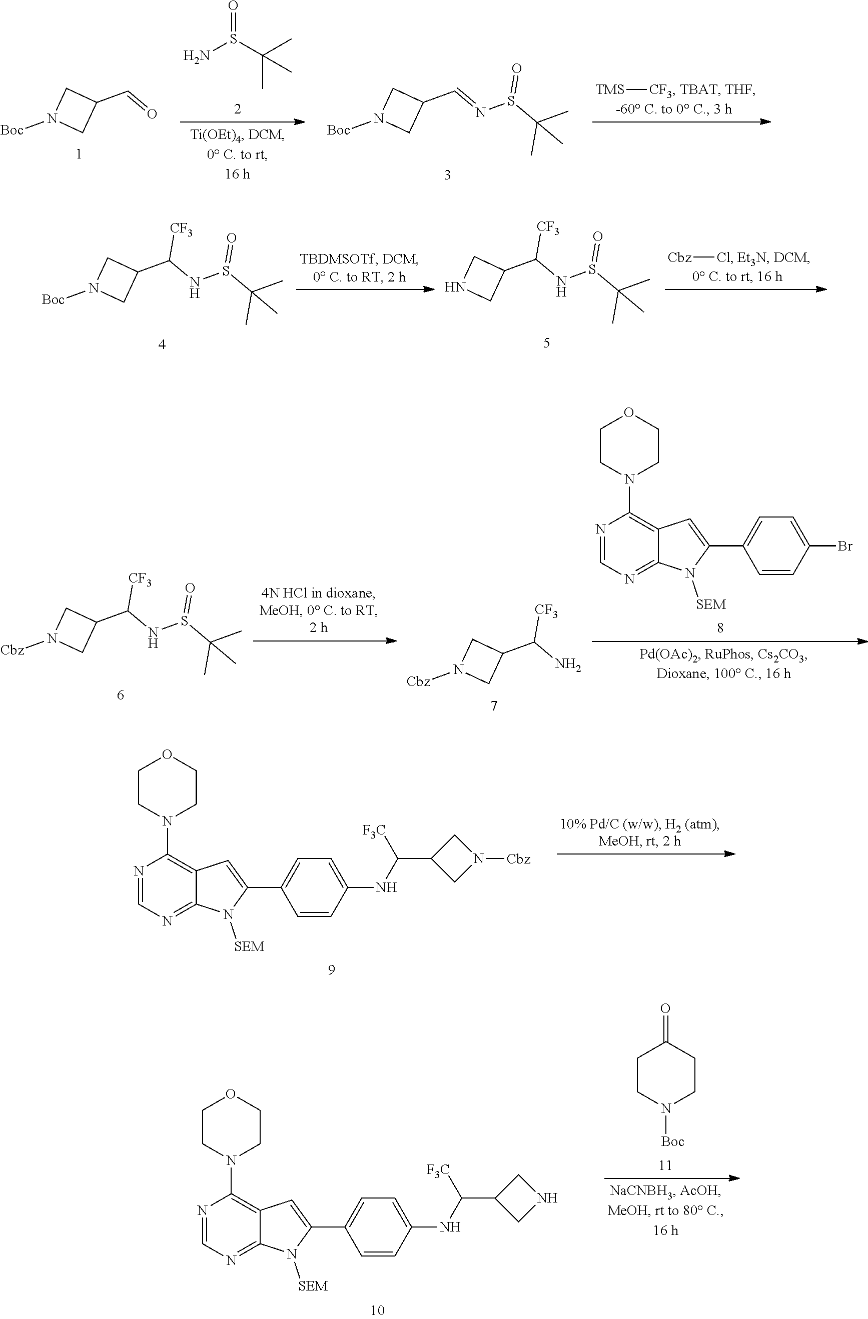

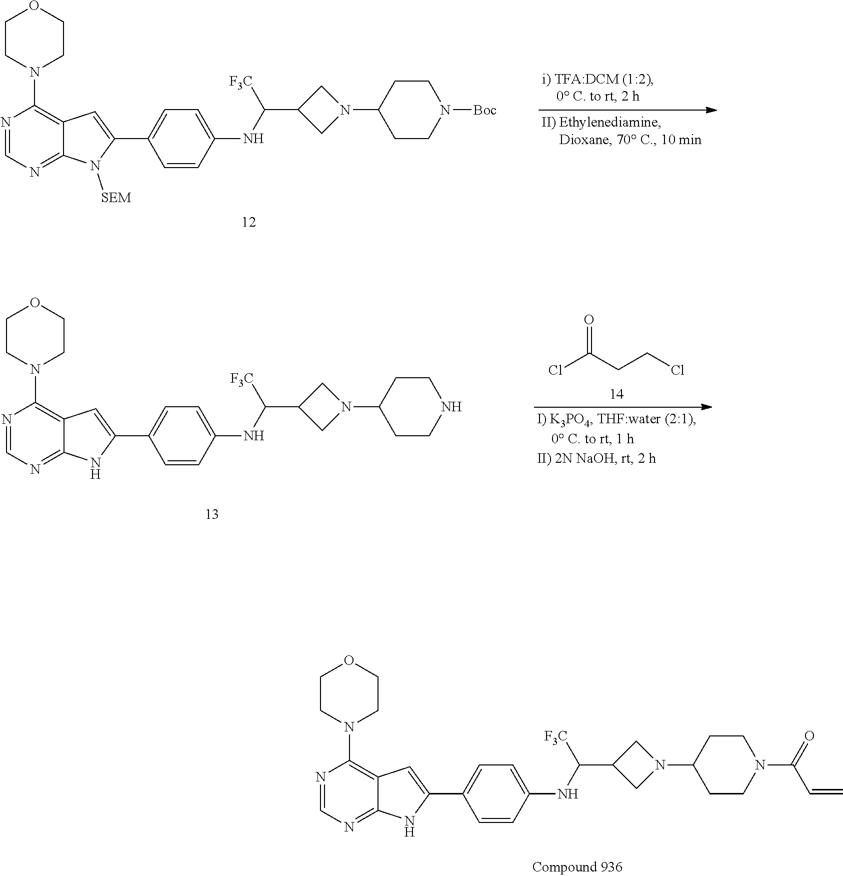 [SMILES: CC(C)(C)OC(=O)N1CCC(N2CC(C(Nc3ccc(-c4cc5c(N6CCOCC6)ncnc5n4COCC[Si](C)(C)C)cc3)C(F)(F)F)C2)CC1 ; FC(F)(F)C(Nc1ccc(-c2cc3c(N4CCOCC4)ncnc3[nH]2)cc1)C1CN(C2CCNCC2)C1 ; O=C(Cl)CCCl ; C=CC(=O)N1CCC(N2CC(C(Nc3ccc(-c4cc5c(N6CCOCC6)ncnc5[nH]4)cc3)C(F)(F)F)C2)CC1]

### Synthesis of tert-butyl (E)-3-(((tert-butylsulfinyl)imino)methyl)azetidine-1-carboxylate (3)

To a stirred solution of tert-butyl 3-formylazetidine-1-carboxylate 1 (10 g, 54 mmol) in DCM (100 mL) were added 2-methylpropane-2-sulfinamide 2 (7.85 g, 64.8 mmol) and Ti (OEt) 4 (23 mL, 108 mmol) sequentially at 0° C. The reaction mixture was stirred at RT for 16 h. After completion of reaction (TLC monitoring), the reaction mixture was quenched with saturated brine solution (100 mL), filtered through celite pad and washed with DCM (100 mL). The filtrate was extracted with DCM (2×100 mL). The combined organic layer was dried over Na₂SO₄, filtered and evaporated under reduced pressure. The crude was purified by flash chromatography (silica gel 100-200 M, 40 g SNAP) using eluents 30-40% EtOAc in heptane to get the title compound as a white solid 3 (12.5 g, 77%).

LCMS=[M+H]⁺: 289.12; Purity=96%.

### Synthesis of tert-butyl 3-(1-((tert-butylsulfinyl)amino)-2,2,2-trifluoroethyl)azetidine-1-carboxylate (4)

To a stirred solution of tert-butyl (E)-3-(((tert-butylsulfinyl)imino)methyl)azetidine-1-carboxylate 3 (12.5 g, 43.3 mmol) in dry THF (150 mL) was added TBAT (25.7 g, 47.7 mmol) under N₂ atmosphere. The reaction mixture was cooled at −65° C., and a solution of TMS-CF₃ (10 mL, 65 mmol) dissolved in dry THF (50 mL) was added slowly and stirred for 1 h. Then the reaction mixture was warmed to 0° C. and stirred for 2 h. After completion of reaction (monitored by TLC), the reaction mixture was quenched with ice cold water (100 mL) and extracted with EtOAc (3×100 mL). The combined organic layer was dried over Na₂SO₄, filtered and evaporated under reduced pressure. The crude was purified by flash chromatography (silica gel 100-200 M, 80 g SNAP) using eluents 25-30% EtOAc in heptane to get the title compound as a white solid 4 (11 g, 67%).

LCMS=[M+H]⁺: 356.9; Purity=95%.

### Synthesis of N-(1-(azetidin-3-yl)-2,2,2-trifluoroethyl)-2-methylpropane-2-sulfinamide (5)

To cold stirred solution f tert-butyl 3-(1-((tert-butylsulfinyl)amino)-2,2,2-trifluoroethyl)azetidine-1-carboxylate 4 (1 g, 2.79 mmol) in DCM (10 mL) was added TBDMSOTf (1.2 mL, 3.35 mmol) at 0° C. under N₂ atmosphere. The reaction was stirred at RT for 2 h. After completion of reaction (monitored by TLC), the reaction mixture was quenched with sat. NaHCO₃ and extracted with 25% IPA in CHCl₃ (3×50 mL). The combined organic layer was dried over Na₂SO₄, filtered and concentrated under reduced pressure to get the title compound as a colorless sticky oil 5 (700 mg; crude) which was used in next step directly without further purification.

LCMS=[M+H]⁺: 258.9; Purity=88%.

### Synthesis of benzyl 3-(1-((tert-butylsulfinyl)amino)-2,2,2-trifluoroethyl)azetidine-1-carboxylate (6)

To a stirred solution of N-(1-(azetidin-3-yl)-2,2,2-trifluoroethyl)-2-methylpropane-2-sulfinamide 5 (700 mg, 2.71 mmol) in DCM (10 mL) were added Et₃N (1.13 mL, 8.13 mmol) and Cbz-Cl (0.6 mL, 4.06 mmol) at 0° C. The reaction was stirred at RT for 16 h. After completion of reaction (monitored by TLC), the reaction mixture was quenched with ice cold water (50 mL) and extracted with DCM (3×50 mL). The combined organic layer was dried over Na₂SO₄, filtered and concentrated under reduced pressure. The crude was purified by flash chromatography (silica gel 100-200 M, 12 g SNAP) using eluents 25-30% EtOAc in heptane to get the title compound as light yellow sticky liquid 6 (600 mg, 56%).

LCMS=[M+H]⁺: 393.12; Purity=72%.

### Synthesis of benzyl 3-(1-amino-2,2,2-trifluoroethyl)azetidine-1-carboxylate (7)

To a stirred solution of benzyl 3-(1-((tert-butylsulfinyl)amino)-2,2,2-trifluoroethyl)azetidine-1-carboxylate 6 (600 mg, 1.53 mmol) in MeOH (6 mL) was added 4N HCl in 1,4-dioxane (4 mL, 15.3 mmol) at 0° C. and stirred at RT for 2 h. After completion of reaction (monitored by TLC), the reaction mixture was concentrated under reduced pressure. The crude was basified with sat. NaHCO₃ to pH~8-9 and extracted with 25% IPA in CHCl₃ (3×50 mL). The combined organic layer was dried over Na₂SO₄, filtered and concentrated under reduced pressure to get the title compound as colorless liquid 7 (480 mg; crude) which was used in next step directly without further purification.

LCMS=[M+H]⁺: 289.05; Purity=72%.

### Synthesis of benzyl 3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)azetidine-1-carboxylate (9)

To a stirred solution of 4-(6-(4-bromophenyl)-7-((2-(trimethylsilyl)ethoxy)methyl)-7H-pyrrolo[2,3-d]pyrimidin-4-yl)morpholine 8 (500 mg, 1.02 mmol) and benzyl 3-(1-amino-2,2,2-trifluoroethyl)azetidine-1-carboxylate 7 (442 mg, 1.53 mmol) in toluene (10 mL) was added Cs₂CO₃ (998 mg, 3.06 mmol) at RT. The reaction mixture was purged with N₂ gas for 10 min, followed by RuPhos (95.3 mg, 0.204 mmol) and Pd (OAc) 2 (22.9 mg, 0.102 mmol) was added, and stirred at 100° C. for 16 h. After completion of reaction (monitored by TLC and LCMS), the reaction mixture was diluted with water (50 mL) and extracted with EtOAc (3×50 mL). The combined organic layer was dried over Na₂SO₄, filtered and evaporated under reduced pressure. The crude was purified by flash chromatography (silica gel 100-200 M, 12 g SNAP) using eluents 20-30% EtOAc in heptane to get the title compound as a brown solid 9 (400 mg, 56%).

LCMS=[M+H]⁺: 697.33; Purity=70%.

### Synthesis of N-(1-(azetidin-3-yl)-2,2,2-trifluoroethyl)-4-(4-morpholino-7-((2-(trimethylsilyl)ethoxy)methyl)-7H-pyrrolo[2,3-d]pyrimidin-6-yl)aniline (10)

To a stirred solution of benzyl 3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)azetidine-1-carboxylate 9 (400 mg, 0.574 mmol) in MeOH (8 mL) was added 10% Pd/C(428 mg) at RT under H₂ atmosphere. The reaction mixture was stirred at RT for 2 h. After completion of reaction (monitored by TLC and LCMS), the reaction mixture was filtered through celite pad and washed with MeOH (20 mL). The filtrate was concentrated under reduced pressure to get the title compound as colorless oil 10 (300 mg; crude) which was used in next

LCMS=[M+H]⁺: 563.28; Purity=77%.

### Synthesis of tert-butyl 4-(3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)azetidin-1-yl)piperidine-1-carboxylate (12)

To a stirred solution of N-(1-(azetidin-3-yl)-2,2,2-trifluoroethyl)-4-(4-morpholino-7-((2-(trimethylsilyl)ethoxy)methyl)-7H-pyrrolo[2,3-d]pyrimidin-6-yl)aniline 10 (300 mg, 0.533 mmol) and tert-butyl 4-oxopiperidine-1-carboxylate 11 (127 mg, 0.640 mmol) in MeOH (5 mL) was added AcOH (cat) at 0° C. The reaction mixture was stirred at 80° C. for 2 h. Then the reaction mixture was cooled to 0° C., and a solution of NaCNBH₃ (67 mg, 1.07 mmol) in MeOH (1 mL) was added and stirred at 80° C. for 16 h. After completion of reaction (monitored by TLC and LCMS), the reaction mixture was quenched with sat. NaHCO₃ (5 mL) extracted with DCM (3×50 mL). The combined organic layer was dried over Na₂SO₄, filtered and concentrated under reduced pressure. The crude was purified by flash chromatography (silica gel 100-200 M, 12 g SNAP) using eluents 5-6.5% MeOH in DCM to get the title compound as a white solid 12 (200 mg, 50%) LCMS=[M+H]⁺: 746.48; Purity=70%.

### Synthesis of 4-(4-morpholino-7H-pyrrolo[2,3-d]pyrimidin-6-yl)-N-(2,2,2-trifluoro-1-(1-(piperidin-4-yl)azetidin-3-yl)ethyl)aniline (13)

To a stirred solution of tert-butyl 4-(3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)azetidin-1-yl)piperidine-1-carboxylate 12 (200 mg, 0.268 mmol) in DCM (3 mL) was added TFA (1.5 mL) dropwise at 0° C. under N₂ atmosphere. The reaction mixture was stirred at RT for 2 h. After completion of reaction (monitored by TLC and LCMS), the reaction mixture was concentrated under reduced pressure, triturated with Et2O and dried under reduced pressure. The obtained crude was dissolved in 1,4-dioxane (3 mL) and ethylenediamine (53 μL, 0.804 mmol) was added and stirred at 70° C. for 10 min. After completion of reaction (monitored by LCMS), the reaction was poured into ice cold water (20 mL) and extracted with 25% IPA in CHCl₃ (3×30 mL). The combined organic layer was dried over Na₂SO₄, filtered and concentrated under reduced pressure to get the title compound as a brown solid 13 (200 mg; crude) which was used in next step directly without further purification.

LCMS=[M+H]⁺: 516.24; Purity=65%.

### Synthesis of 1-(4-(3-(2,2,2-trifluoro-1-((4-(4-morpholino-7H-pyrrolo[2,3-d]pyrimidin-6-yl)phenyl)amino)ethyl)azetidin-1-yl)piperidin-1-yl)prop-2-en-1-one (Compound 936)

To an ice cold stirred solution of 4-(4-morpholino-7H-pyrrolo[2,3-d]pyrimidin-6-yl)-N-(2,2,2-trifluoro-1-(1-(piperidin-4-yl)azetidin-3-yl)ethyl)aniline 13 (200 mg, 0.388 mmol) in THF (4 mL) was added K₃PO₄ (165 mg, 0.776 mmol) dissolved in water (2 mL) under N₂ atmosphere and stirred for 5 min. Then a solution of 3-chloropropionyl chloride 14 (36 μL, 0.388 mmol) in THF (1 mL) was added drop wise to the reaction mixture at 0° C. and stirred at RT for 1 h. After completion of starting material (acylation reaction, monitored by TLC), 2N aq. NaOH (2 mL) was added to the reaction mixture at 0° C. and stirred at RT for 2 h. After completion of reaction (complete elimination, monitored by TLC and LCMS), the reaction mixture was diluted with water (10 mL) and extracted with 2-MeTHF (3×50 mL). The combined organic layer was dried over Na₂SO₄, filtered and concentrated under reduced pressure. The crude was purified by flash column chromatography (silica gel 100-200 M, 4 g SNAP) using eluents 7-10% MeOH in DCM to get the title Compound 936 (32 mg, 14%).

¹H NMR (400 MHz, DMSO-d₆): δ 11.97 (s, 1H), 8.13 (s, 1H), 7.65 (d, J=8.4 Hz, 2H), 6.89 (s, 1H), 6.84 (d, J=8.4 Hz, 2H), 6.79-6.73 (m, 1H), 6.17-6.15 (m, 1H), 6.07-6.02 (m, 1H), 5.63-5.61 (m, 1H), 4.55-4.52 (m, 1H), 3.95-3.92 (m, 1H), 3.85-3.80 (m, 4H), 3.74-3.73 (m, 4H), 3.40-3.37 (m, 1H), 3.17-3.15 (m, 1H), 2.98-2.80 (m, 2H), 2.75-2.74 (m, 1H), 2.67-2.64 (m, 1H), 2.32-2.27 (m, 1H), 1.62-1.58 (m, 2H), 1.08 (t, J=7.2 Hz, 4H).

LCMS=[M+H]⁺: 570.24; Purity=93.71%.

### Example 87

### Compound 937

### Preparation of 1-(4-(5-((4-(4-morpholino-1H-pyrrolo[3,2-c]pyridin-2-yl)phenyl)amino)pyrimidin-2-yl)piperazin-1-yl)prop-2-en-1-one (Compound 937)

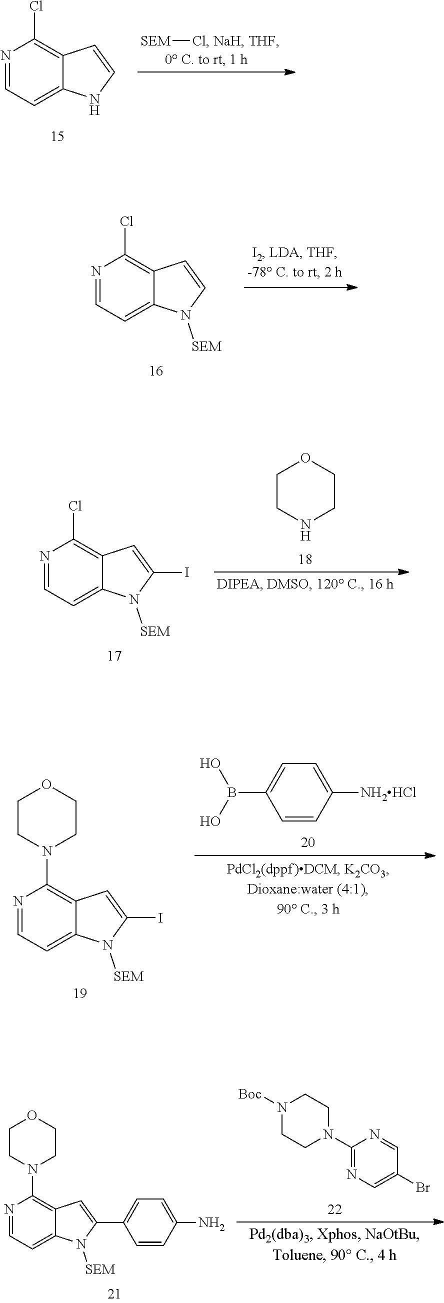

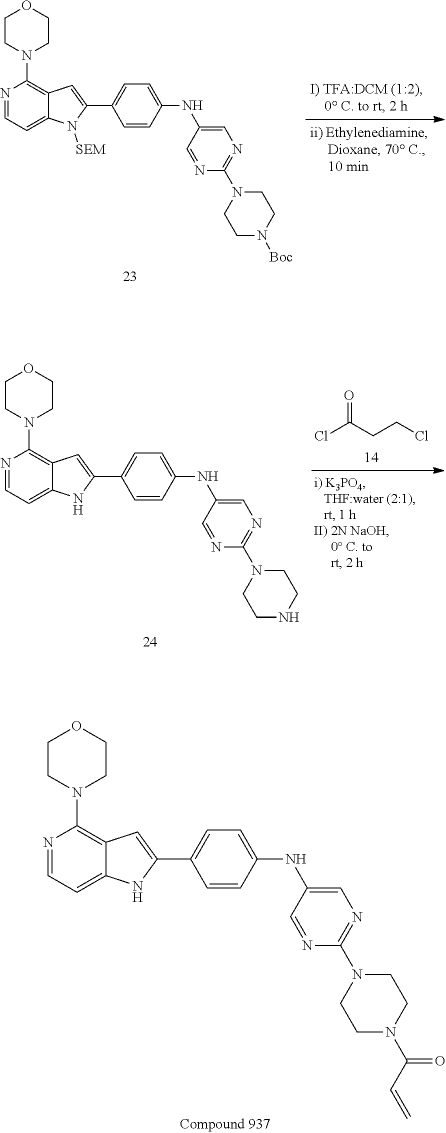 [SMILES: CC(C)(C)OC(=O)N1CCN(c2ncc(Nc3ccc(-c4cc5c(N6CCOCC6)nccc5n4COCC[Si](C)(C)C)cc3)cn2)CC1 ; c1cc2[nH]c(-c3ccc(Nc4cnc(N5CCNCC5)nc4)cc3)cc2c(N2CCOCC2)n1 ; O=C(Cl)CCCl ; C=CC(=O)N1CCN(c2ncc(Nc3ccc(-c4cc5c(N6CCOCC6)nccc5[nH]4)cc3)cn2)CC1]

### Synthesis of 4-chloro-1-((2-(trimethylsilyl)ethoxy)methyl)-1H-pyrrolo[3,2-c]pyridine (16)

To a stirred solution of 4-chloro-1H-pyrrolo[3,2-c]pyridine 15 (10 g, 65.5 mmol) in dry THF (100 mL) was added NaH (5.72 g, 131 mmol; 60% dispersion in mineral oil) at 0° C. and stirred for 30 min. Then SEMCl (17.4 mL, 98.3 mmol) was added dropwise to the reaction mixture at 0° C. and stirred at RT for 1 h. After completion of reaction (monitored by TLC and LCMS), the reaction mixture was quenched with ice cold water (100 mL) and extracted with EtOAc (3×100 mL). The combined organic layer was dried over Na₂SO₄, filtered and concentrated under reduce pressure. The crude was purified by flash column chromatography (silica gel 100-200 mesh, 40 g SNAP) using eluents 5-10% EtOAc in heptane to get the desired compound as colorless oil 16 (9.8 g, 52%).

LCMS=[M+H]⁺: 283.06; Purity=98%.

### Synthesis of 4-chloro-2-iodo-1-((2-(trimethylsilyl)ethoxy)methyl)-1H-pyrrolo[3,2-c]pyridine (17)

To a stirred solution of 4-chloro-1-((2-(trimethylsilyl)ethoxy)methyl)-1H-pyrrolo[3,2-c]pyridine 16 (5.9 g, 20.9 mmol) in THF (60 mL) was added LDA (15.6 mL, 31.3 mmol, 2M in THF) dropwise at −78° C. under N₂ atmosphere and stirred for 1 h. Then a solution of I₂ (6.4 g, 25 mmol) in dry THF (20 mL) was added drop wise at −78° C. and stirred at RT for 2 h. After completion of reaction (monitored by TLC), the reaction mixture was cooled to 0° C., quenched with sat. NH₄Cl solution (100 mL) and extracted with EtOAc (3×100 mL). The combined organic layer was dried over Na₂SO₄, filtered and evaporated under reduced pressure. The crude was purified by flash column chromatography (silica gel, 100-200 M, 40 g SNAP) using eluents 5-10% EtOAc in heptane to get the desired compound as a brown solid 17 (6.8 g, 70%).

LCMS=[M+H]⁺: 408.98; Purity=88%.

### Synthesis of 4-(2-iodo-1-((2-(trimethylsilyl)ethoxy)methyl)-1H-pyrrolo[3,2-c]pyridin-4-yl)morpholine (19)

To a stirred solution of 4-chloro-2-iodo-1-((2-(trimethylsilyl)ethoxy)methyl)-1H-pyrrolo[3,2-c]pyridine 17 (6.7 g, 16.4 mmol) in DMSO (60 mL) were added DIPEA (14.5 mL, 82 mmol) and morpholine 18 (7 mL, 82 mmol) at RT. The reaction mixture was stirred at 120° C. for 16 h. After completion of reaction (monitored by TLC), the reaction mixture was cooled to RT, poured into ice cold-water (100 mL) and extracted with EtOAc (2×100 mL). The combined organic layer was washed with ice cold brine (2×100 mL), dried over Na₂SO₄, filtered and evaporated under reduced pressure. The crude was purified by flash column chromatography (silica gel 100-200 M, 40 g SNAP) using eluents 10-15% EtOAc in heptane to get the title compound as a brown solid 19 (4 g, 53%).

LCMS=[M+H]⁺: 460.13; Purity=93%.

### Synthesis of 4-(4-morpholino-1-((2-(trimethylsilyl)ethoxy)methyl)-1H-pyrrolo[3,2-c]pyridin-2-yl)aniline (21)

To a stirred solution of 4-(2-iodo-1-((2-(trimethylsilyl)ethoxy)methyl)-1H-pyrrolo[3,2-c]pyridin-4-yl)morpholine 19 (2.9 g, 6.31 mmol) and (4-aminophenyl) boronic acid hydrochloride 20 (1.64 g, 9.47 mmol) in 1,4-dioxane (40 mL) and water (10 mL) was added K₂CO₃ (2.62 g, 18.9 mmol) at RT. The reaction mixture was degassed with N₂ gas for 15 min, followed by PdCl₂(dppf)·DCM (515 mg, 0.631 mmol) was added and stirred at 90° C. for 3 h. After completion of reaction (monitored by TLC and LCMS), the reaction mixture was filtered through celite pad and washed with EtOAc (100 mL). The filtrate was diluted with water (50 mL) and extracted with EtOAc (3×100 mL). The combined organic layer was dried over Na₂SO₄, filtered and evaporated under reduced pressure. The crude was purified by flash column chromatography (silica gel 100-200 M, 40 g SNAP) using eluents 30-40% EtOAc in heptane to get the title compound as a light brown solid 21 (2.3 g, 60%).

LCMS=[M+H]⁺: 425.25; Purity=71%.

### Synthesis of tert-butyl 4-(5-((4-(4-morpholino-1-((2-(trimethylsilyl)ethoxy)methyl)-1H-pyrrolo[3,2-c]pyridin-2-yl)phenyl)amino)pyrimidin-2-yl)piperazine-1-carboxylate (23)

To a stirred solution of tert-butyl 4-(5-bromopyrimidin-2-yl)piperazine-1-carboxylate 22 (1 g, 2.91 mmol) and 4-(4-morpholino-1-((2-(trimethylsilyl)ethoxy)methyl)-1H-pyrrolo[3,2-c]pyridin-2-yl)aniline 21 (1.48 g, 3.5 mmol) in toluene (15 mL) was added NaO^{t}Bu (420 mg, 4.37 mmol) at RT. The reaction mixture was degassed with N₂ gas for 10 min, followed by Pd₂(dba)₃ (267 mg, 0.291 mmol) and XPhos (139 mg, 0.291 mmol). The reaction mixture was stirred at 90° C. for 4 h. After completion of reaction (monitored by TLC and LCMS), the reaction mixture was diluted with water (60 mL) and extracted with EtOAc (3×80 mL). The combined organic layer was dried over Na₂SO₄, filtered and evaporated under reduced pressure. The crude was purified by flash column chromatography (silica gel 100-200 M, 24 g SNAP) using eluents 20-30% EtOAc in heptane to get the title compound as a brown solid 23 (1.6 g, 28%).

LCMS=[M+H]⁺: 687.41; Purity=70%.

### Synthesis of N-(4-(4-morpholino-1H-pyrrolo[3,2-c]pyridin-2-yl)phenyl)-2-(piperazin-1-yl)pyrimidin-5-amine (24)

To a stirred solution of tert-butyl 4-(5-((4-(4-morpholino-1-((2-(trimethylsilyl)ethoxy)methyl)-1H-pyrrolo[3,2-c]pyridin-2-yl)phenyl)amino)pyrimidin-2-yl)piperazine-1-carboxylate 23 (800 mg, 1.16 mmol) in DCM (8 mL) was added TFA (4 mL) dropwise at 0° C. and stirred at RT for 2 h. After completion of reaction (monitored by TLC), the reaction mixture was concentrated under reduced pressure, triturated with Et₂O and dried under reduced pressure. The obtained crude was dissolved in 1,4-dioxane (8 mL) and ethylenediamine (0.12 mL, 1.75 mmol) was added and stirred at 70° C. for 10 min. After completion of reaction (monitored by LCMS), the reaction was poured into ice cold water (50 mL) and extracted with 25% IPA in CHCl₃ (3×50 mL). The combined organic layer was dried over Na₂SO₄, filtered and concentrated under reduced pressure to get the title compound as a brown solid 24 (500 mg; crude) which was used in next step directly without further purification.

LCMS=[M+H]⁺: 457.19; Purity=55%.

### Synthesis of 1-(4-(5-((4-(4-morpholino-1H-pyrrolo[3,2-c]pyridin-2-yl)phenyl)amino)pyrimidin-2-yl)piperazin-1-yl)prop-2-en-1-one (Compound 937)

To a stirred solution of N-(4-(4-morpholino-1H-pyrrolo[3,2-c]pyridin-2-yl)phenyl)-2-(piperazin-1-yl)pyrimidin-5-amine 24 (500 mg, 1.1 mmol) in THF (6 mL) was added K₃PO₄ (465 mg, 2.19 mmol) dissolved with water (3 mL) at 0° C. and stirred for 5 min. Then a solution of 3-chloropropanoyl chloride 14 (139 mg, 1.1 mmol) in THF (1 mL) was added dropwise and stirred at RT for 1 h. After completion of starting material (acylation reaction, monitored by TLC), 2N aq. NaOH (6.5 mL, 13.1 mmol) was added to the reaction mixture at 0° C. and stirred at RT for 2 h. After completion of reaction (complete elimination, monitored by TLC and LCMS), the reaction mixture was diluted with water (30 mL) and extracted with 2-MeTHF (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X select CSH C18 (19*250) 10μ] to get the title Compound 937 (71 mg, 12%).

¹H NMR (400 MHz, DMSO-d₆): $11.59 (s, 1H), 8.34 (s, 2H), 7.95 (s, 1H), 7.71-7.66 (m, 3H), 6.89-6.82 (m, 5H), 6.15 (dd, J=16.8 Hz, 2.4 Hz, 1H), 5.72 (dd, J=10.4 Hz, 2.4 Hz, 1H), 3.78-3.76 (m, 4H), 3.75-3.71 (m, 4H), 3.68-3.62 (m, 4H), 3.54-6.52 (m, 4H).

LCMS=[M+H]⁺: 511.19; Purity=98%.

### Example 88

### Compound 938

### Preparation of 1-(4-(2-((4-(4-morpholino-7H-pyrrolo[2,3-d]pyrimidin-6-yl)phenyl)amino)pyrimidin-5-yl)piperazin-1-yl)prop-2-en-1-one (Compound 938)

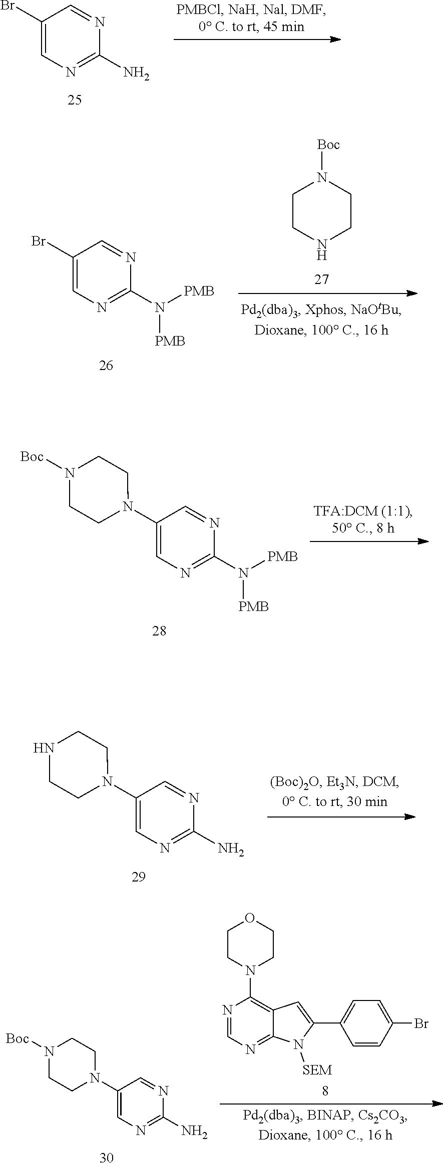

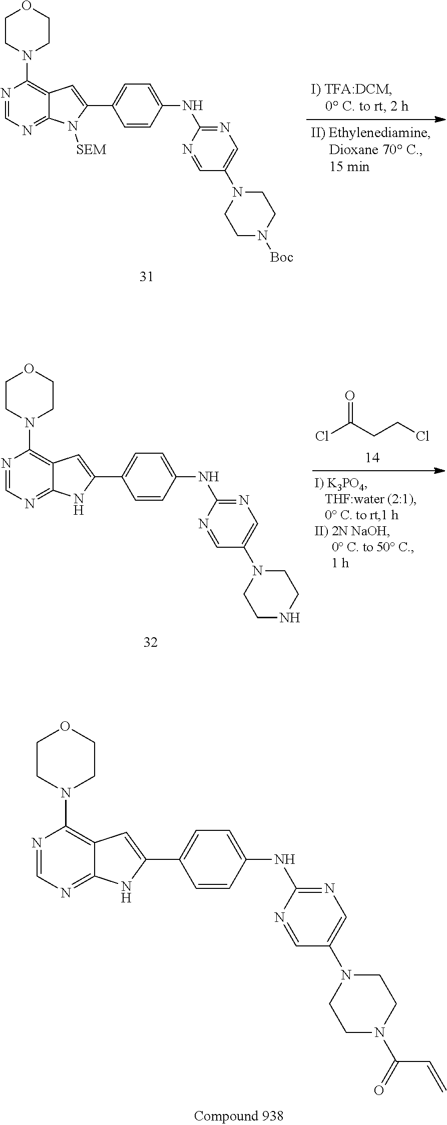 [SMILES: CC(C)(C)OC(=O)N1CCN(c2cnc(Nc3ccc(-c4cc5c(N6CCOCC6)ncnc5n4COCC[Si](C)(C)C)cc3)nc2)CC1 ; c1nc(N2CCOCC2)c2cc(-c3ccc(Nc4ncc(N5CCNCC5)cn4)cc3)[nH]c2n1 ; O=C(Cl)CCCl ; C=CC(=O)N1CCN(c2cnc(Nc3ccc(-c4cc5c(N6CCOCC6)ncnc5[nH]4)cc3)nc2)CC1]

### Synthesis of 5-bromo-N,N-bis(4-methoxybenzyl)pyrimidin-2-amine (26)

To a stirred solution of 5-bromopyrimidin-2-amine 25 (5 g, 28.7 mmol) in dry DMF (50 mL) were added NaH (3.13 g, 71.8 mmol; 60% dispersion in mineral oil) and NaI (431 mg, 2.87 mmol) at 0° C. under N₂ atmosphere and stirred for 15 min. Then PMB-Cl (7.8 mL, 57.5 mmol) was added to the reaction mixture and stirred at RT for 45 min. After completion of reaction (monitored by TLC and LCMS), the reaction mixture was quenched with ice cold water (100 mL) and extracted with EtOAc (3×100 mL). The combined organic layer was washed with ice cold brine (2×100 mL), dried over Na₂SO₄, filtered and concentrated under reduce pressure. The crude was purified by flash column chromatography (silica gel 100-200 mesh, 40 g SNAP) using eluents 5-10% EtOAc in heptane to get the desired compound as a white solid 26 (11 g, 92%).

LCMS=[M+H]⁺: 414.04; Purity=98%.

### Synthesis of tert-butyl 4-(2-(bis(4-methoxybenzyl)amino)pyrimidin-5-yl)piperazine-1-carboxylate (28)

To a stirred solution of 5-bromo-N,N-bis(4-methoxybenzyl)pyrimidin-2-amine 26 (3.5 g, 8.45 mmol) and tert-butyl piperazine-1-carboxylate 27 (2.36 g, 12.7 mmol) in 1,4-Dioxane (40 mL) was added NaO^{t}Bu (1.62 g, 16.9 mmol) at RT. The reaction mixture was added purged with N₂ for 15 min, followed by Pd₂(dba)₃ (684 mg, 0.845 mmol) and XPhos (805 mg, 1.69 mmol) were added at RT. The reaction mixture was stirred at 100° C. for 16 h. After completion of reaction (monitored by TLC and LCMS), the reaction mixture was filtered through celite pad and washed with EtOAc (100 mL). The filtrate was diluted with water (100 mL) and extracted with EtOAc (2×100 mL). The combined organic layer was dried over Na₂SO₄, filtered and evaporated under reduced pressure. The crude was purified by flash column chromatography (silica gel, 100-200 mesh, 12 g SNAP) using eluents 20-30% EtOAc in heptane to get the title compound as a yellow sticky solid 28 (3 g, 68%).

LCMS=[M+H]⁺: 520.36; Purity=70%.

### Synthesis of 5-(piperazin-1-yl)pyrimidin-2-amine (29)

To a stirred solution of tert-butyl 4-(2-(bis(4-methoxybenzyl)amino)pyrimidin-5-yl)piperazine-1-carboxylate 28 (3 g, 5.78 mmol) in DCM (25 mL) was added TFA (25 mL) dropwise at 0° C. and stirred at 50° C. for 8 h. After completion of reaction (monitored by TLC and LCMS), the reaction mixture was concentrated under reduced pressure, triturated with Et₂O and dried under reduced pressure to get the title compound as a brown sticky solid 29 (1 g; crude) which was used in next step without further purification.

LCMS=[M−H]: 178.12; Purity=50%.

### Synthesis of tert-butyl 4-(2-aminopyrimidin-5-yl)piperazine-1-carboxylate (30)

To a stirred solution of 5-(piperazin-1-yl)pyrimidin-2-amine 29 (1 g, 5.58 mmol) in DCM (10 mL) was added Et₃N (1.2 mL 8.37 mmol) at 0° C. and stirred for 5 min, followed by (Boc) 20 (1.6 mL, 6.7 mmol) was added to the reaction mixture at 0° C. and stirred at RT for 30 min. After completion of reaction (monitored by TLC), the reaction mixture was concentrated under reduced pressure. The crude was purified by flash column chromatography (silica gel 100-200 M, 12 g SNAP) using eluents 8-10% MeOH in DCM to get the title compound as a brown sticky solid 30 (1.5 g, 96%).

LCMS=[M+H]⁺: 280.19; Purity=93%.

### Synthesis of tert-butyl 4-(2-((4-(4-morpholino-7-((2-(trimethylsilyl)ethoxy)methyl)-7H-pyrrolo[2,3-d]pyrimidin-6-yl)phenyl)amino)pyrimidin-5-yl)piperazine-1-carboxylate (31)

To a stirred solution of 4-(6-(4-bromophenyl)-7-((2-(trimethylsilyl)ethoxy)methyl)-7H-pyrrolo[2,3-d]pyrimidin-4-yl)morpholine 8 (1.75 g, 3.58 mmol) and tert-butyl 4-(2-aminopyrimidin-5-yl)piperazine-1-carboxylate 30 (1 g, 3.58 mmol) in 1,4-Dioxane (10 mL) was added Cs₂CO₃ (2.33 g, 7.16 mmol) at RT. The reaction mixture was purged with N₂ for 15 min, followed by Pd₂(dba)₃ (328 mg, 0.358 mmol) and BINAP (446 mg, 0.716 mmol) were added at RT. The reaction mixture was stirred at 100° C. for 16 h. After completion of reaction (monitored by TLC and LCMS), the reaction mixture was filtered through celite pad and washed with EtOAc (100 mL). The filtrate was diluted with water (50 mL) and extracted with EtOAc (2×100 mL). The combined organic layer was dried over Na₂SO₄, filtered and evaporated under reduced pressure. The crude was purified by flash column chromatography (silica gel 100-200 M, 12 g SNAP) using 20-30% EtOAc in heptane to get the title compound as a yellow sticky solid 31 (400 mg, 16%).

LCMS=[M+H]⁺: 688.37; Purity=60%.

### Synthesis of N-(4-(4-morpholino-7H-pyrrolo[2,3-d]pyrimidin-6-yl)phenyl)-5-(piperazin-1-yl)pyrimidin-2-amine (32)

To a stirred solution of tert-butyl 4-(2-((4-(4-morpholino-7-((2-(trimethylsilyl)ethoxy)methyl)-7H-pyrrolo[2,3-d]pyrimidin-6-yl)phenyl)amino)pyrimidin-5-yl)piperazine-1-carboxylate 31 (400 mg, 0.221 mmol) in DCM (5 mL) was added TFA (2.5 mL) dropwise at 0° C. and stirred at RT for 2 h. After completion of reaction (monitored by TLC), the reaction mixture was concentrated under reduced pressure, triturated with Et₂O and dried under reduced pressure. The obtained crude was dissolved in 1,4-dioxane (4 mL) and ethylenediamine (25 μL, 0.331 mmol) was added and stirred at 70° C. for 15 min. After completion of reaction (monitored by LCMS), the reaction was poured into ice cold water (50 mL) and extracted with 25% IPA in CHCl₃ (3×50 mL). The combined organic layer was dried over Na₂SO₄, filtered and concentrated under reduced pressure to get the title compound as a brown solid 32 (140 mg; crude) which was used in next step directly without further purification.

LCMS=[M+H]⁺: 458.23; Purity=83%.

### Synthesis of 1-(4-(2-((4-(4-morpholino-7H-pyrrolo[2,3-d]pyrimidin-6-yl)phenyl)amino)pyrimidin-5-yl)piperazin-1-yl)prop-2-en-1-one (Compound 938)

To an ice cold stirred solution of N-(4-(4-morpholino-7H-pyrrolo[2,3-d]pyrimidin-6-yl)phenyl)-5-(piperazin-1-yl)pyrimidin-2-amine 32 (120 mg, 0.262 mmol) in THF (3 mL) was added K₃PO₄ (111 mg, 0.525 mmol) dissolved in water (1.5 mL) at 0° C. and stirred for 5 min. Then a solution of 3-chloropropanoyl chloride 14 (25 μL, 0.262 mmol) in THF (0.5 mL) was added drop wise and stirred at RT for 1 h. After completion of starting material (acylation reaction, monitored by TLC), 2N aq. NaOH (1.6 mL, 3.15 mmol) was added to the reaction mixture at 0° C. and stirred at 50° C. for 1 h. After completion of reaction (complete elimination, monitored by TLC and LCMS), the reaction mixture was diluted with water (30 mL) and extracted with 2-MeTHF (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X B C18 (19*250) 10μ] to get the title Compound 938 (32 mg, 24%).

¹H NMR (400 MHz, DMSO-d₆): δ 12.09 (brs, 1H), 9.50 (s, 1H), 8.34 (s, 2H), 8.16 (s 1H), 7.79 (s, 4H), 7.00 (s, 1H) 6.87 (dd, J=16.8, 10.4 Hz, 1H), 6.14 (dd, J=16.8, 2.4 Hz, 1H), 5.71 (dd, J=10.4, 2.4 Hz, 1H), 3.88-3.85 (m, 4H), 3.76-3.69 (m, 8H), 3.10 (brs, 4H).

LCMS=[M+H]⁺: 512.20; Purity=98.98%.

### Example 89

### Compound 939

### Preparation of 1-(3-(4-((R)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 939)

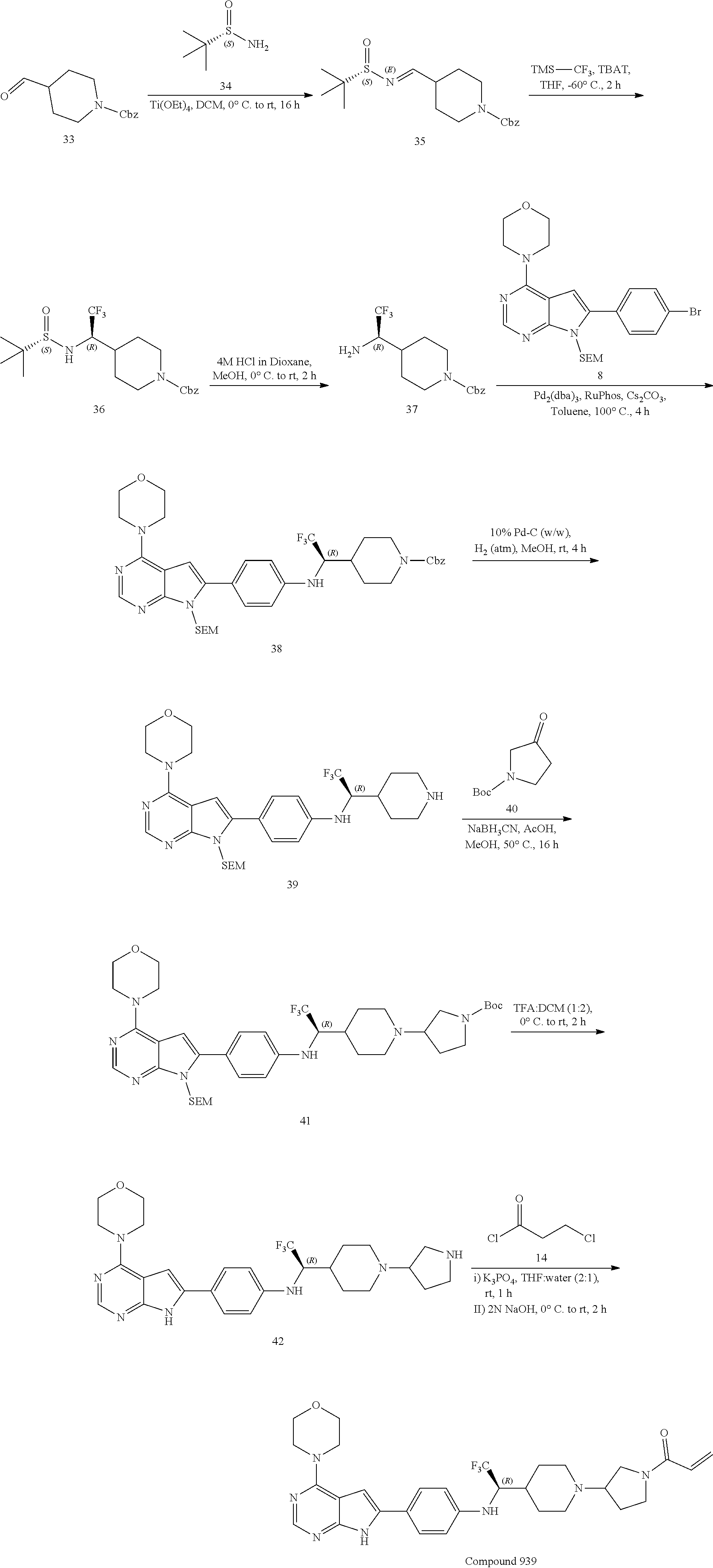

### Synthesis of benzyl (S,E)-4-(((tert-butylsulfinyl)imino)methyl)piperidine-1-carboxylate (35)

To a stirred solution of benzyl 4-formylpiperidine-1-carboxylate 33 (10 g, 40.4 mmol) in DCM (100 mL) was added(S)-2-methylpropane-2-sulfinamide 34 (5.88 g, 48.5 mmol) at 0° C. under N₂ atmosphere, followed by Ti (OEt) 4 (17.3 mL, 80.9 mmol) was added and stirred at RT for 16 h. After completion of reaction (monitored by TLC), the reaction mixture was quenched with brine (100 mL), filtered through celite pad and washed with DCM (100 mL). The filtrate extracted with DCM (3×100 mL). The combined organic layer was dried over Na₂SO₄, filtered and evaporated under reduced pressure. The crude was purified by flash chromatography (silica gel 100-200 M, 120 g SNAP) using eluents 25-30% EtOAc in heptane to afford the tittle compound as a colorless sticky oil 35 (13 g, 81%) LCMS=[M+H]⁺: 351.19; Purity=88%.

### Synthesis of benzyl 4-((R)-1-(((S)-tert-butylsulfinyl)amino)-2,2,2-trifluoroethyl)piperidine-1-carboxylate (36)

To a stirred solution of benzyl (S,E)-4-(((tert-butylsulfinyl)imino)methyl)piperidine-1-carboxylate 35 (13 g, 37.1 mmol) in dry THF (120 mL) was added TBAT (22 g, 40.8 mmol) under N₂ atmosphere. The reaction mixture was cooled at −60° C., and a solution of TMS-CF₃ (11 mL, 74.2 mmol) dissolved in dry THF (20 mL) was added slowly and stirred for 2 h. After completion of reaction (monitored by TLC), the reaction mixture was quenched with ice cold water (150 mL) and extracted with EtOAc (3×150 mL). The combined organic layer was dried over Na₂SO₄, filtered and evaporated under reduced pressure. The crude was purified by flash chromatography (silica gel 100-200 M, 120 g SNAP) using eluents 25-30% EtOAc in heptane to get the title compound as colorless sticky oil 36 (11 g, 69%).

LCMS=[M+H]⁺: 421.7; Purity=98%.

### Synthesis of benzyl (R)-4-(1-amino-2,2,2-trifluoroethyl)piperidine-1-carboxylate (37)

To a stirred solution of benzyl 4-((R)-1-(((S)-tert-butylsulfinyl)amino)-2,2,2-trifluoroethyl)piperidine-1-carboxylate 36 (5 g, 11.9 mmol) in MeOH (50 mL) was added 4N HCl in 1,4-dioxane (30 mL, 119 mmol) dropwise at 0° C. and stirred at RT for 2 h. After completion of reaction (monitored by TLC), the reaction mixture was concentrated under reduced pressure. The crude was diluted with water (20 mL), basified with sat. NaHCO₃ to pH~8-9 and extracted with 25% IPA in CHCl₃ (3×100 mL). The combined organic layer was dried over Na₂SO₄, filtered and concentrated under reduced pressure to get the title compound as sticky oil 37 (3.5 g; crude) which was used in next step directly without further purification.

LCMS=[M+H]⁺: 317.20; Purity=65%.

### Synthesis of benzyl (R)-4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidine-1-carboxylate (38)

To a stirred solution of (2-{[2-(p-bromophenyl)-4-morpholino-1H-1,5,7-triazainden-1-yl]methoxy}ethyl)tris(methyl) silane 8 (1.8 g, 3.68 mmol) and benzyl 4-[(R)-1-amino-2,2,2-trifluoroethyl]-1-piperidinecarboxylate 37 (1.74 g, 5.52 mmol) in toluene (20 mL) was added Cs₂CO₃ (2.4 g, 7.35 mmol) at RT. The reaction mixture was degassed with N₂ gas for 10 min, followed by Pd₂(dba)₃ (336 mg, 0.368 mmol) and RuPhos (343 mg, 0.735 mmol) were added and stirred at 100° C. for 4 h. After completion of reaction (monitored by TLC and LCMS), the reaction mixture was diluted with water (50 mL) and extracted with EtOAc (3×80 mL). The combined organic layer was dried over Na₂SO₄, filtered and concentrated under reduced pressure. The crude was purified by flash column chromatography (silica gel 100-200 M, 40 g SNAP) using eluents 25-35% EtOAc in heptane to get the title compound as a brown semi solid 38 (2 g, 75%).

LCMS=[M+H]⁺: 725.46; Purity=72%.

### Synthesis of (R)-4-(4-morpholino-7-((2-(trimethylsilyl)ethoxy)methyl)-7H-pyrrolo[2,3-d]pyrimidin-6-yl)-N-(2,2,2-trifluoro-1-(piperidin-4-yl)ethyl)aniline (39)

To a solution stirred of benzyl (R)-4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidine-1-carboxylate 38 (1.9 g, 2.62 mmol) in MeOH (30 mL) was added 10% Pd—C(1.95 g) at RT under H₂ atmosphere pressure and stirred at RT for 4 h. After completion of reaction (monitored by TLC and LCMS), the reaction mixture was filtered through celite pad and washed with MeOH (100 mL). The filtrate was concentrated under reduced pressure to get the title compound as a yellow solid 39 (1.5 g; crude) which was used in next step directly without further purification.

LCMS=[M+H]⁺: 591; Purity=85%.

### Synthesis of tert-butyl 3-(4-((R)-2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidin-1-yl)pyrrolidine-1-carboxylate (41)

To a stirred solution of [(R)-2,2,2-trifluoro-1-(4-piperidyl)ethyl][p-(4-morpholino-1-{[2-(trimethylsilyl) ethoxy]methyl}-1H-1,5,7-triazainden-2-yl)phenyl]amine 39 (1.5 g, 2.54 mmol) and tert-butyl 3-oxo-1-pyrrolidinecarboxylate 40 (705 mg, 3.81 mmol) in MeOH (20 mL) was added AcOH (290 μL, 5.08 mmol) at 0° C. under N₂ atmosphere and stirred at RT for 2 h. Then the reaction mixture was cooled to 0° C. and NaBH₃CN (304 mg, 5.08 mmol) was added and stirred at 80° C. for 16 h. After completion of reaction (monitored by TLC and LCMS), the reaction mixture was evaporated under reduced pressure. The crude was basified with sat. NaHCO₃ (100 mL) and extracted with EtOAc (3×100 mL). The combined organic layer was dried over Na2SO₄, filtered and concentrated under reduced pressure. The crude was purified by flash column chromatography (silica gel 100-200 M, 40 g SNAP) using eluents 70-75% EtOAc in heptane to get the title compound as a brown semi solid 41 (900 mg, 46%).

LCMS=[M+H]⁺: 760.40; Purity=72%.

### Synthesis of 4-(4-morpholino-7H-pyrrolo[2,3-d]pyrimidin-6-yl)-N-((1R)-2,2,2-trifluoro-1-(1-(pyrrolidin-3-yl)piperidin-4-yl)ethyl)aniline (42)

To a of tert-butyl 3-{4-[(R)-2,2,2-trifluoro-1-[p-(4-morpholino-1-{[2-stirred solution (trimethylsilyl) ethoxy]methyl}-1H-1,5,7-triazainden-2-yl)phenylamino]ethyl]-1-piperidyl}-1-pyrrolidinecarboxylate 41 (900 mg, 1.18 mmol) in DCM (10 mL) was added TFA (5 mL) dropwise at 0° C. under N₂ atmosphere and stirred at RT for 2 h. After completion of reaction (monitored TLC), the reaction mixture was concentrated under reduced pressure. The crude was basified with sat. NaHCO₃ to pH~8-9 and extracted with 25% IPA in CHCl₃ (3×50 mL). The combined organic layer was dried under over Na₂SO₄, filtered and concentrated under reduced pressure to get the title compound as a brown solid 42 (560 mg; crude) which was used in next step without purification.

LCMS=[M+H]⁺: 530.40; Purity=72%.

### Synthesis of 1-(3-(4-((R)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 939)

To a stirred solution of 4-(4-morpholino-7H-pyrrolo[2,3-d]pyrimidin-6-yl)-N-((1R)-2,2,2-trifluoro-1-(1-(pyrrolidin-3-yl)piperidin-4-yl)ethyl)aniline 42 (550 mg, 0.571 mmol) in THF (6 mL) was added K₃PO₄ (606 mg, 2.86 mmol) dissolved in water (3 mL) at 0° C. under N₂ atmosphere and stirred for 5 min. Then a solution of 3-3-chloropropionyl chloride 14 (87 mg, 0.685 mmol) in THF (1 mL) was added dropwise and stirred at RT for 1 h. After completion of starting material (acylation reaction, monitored by TLC), 2N aq. NaOH (3.4 mL, 6.85 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [2-98% 0.1% TFA in water/ACN; Column: SunFire C18 (19*250) 10μ] to get the title Compound 939 (30 mg, 10%).

¹H NMR (400 MHz, DMSO-d₆): δ 12.21 (brs, 1H), 9.93 (brs, 1H), 8.19 (s, 1H), 7.68 (d, J=8.8 Hz, 2H), 6.95 (brs, 1H), 6.91 (d, J=8.4 Hz, 2H) 6.61-6.53 (m, 1H), 6.30-6.27 (m, 1H), 6.18-6.13 (m, 1H), 5.77-5.70 (m, 1H), 4.54 (brs, 1H), 4.00 (brs, 1H), 3.88-3.83 (m, 4H), 3.77-3.31 (m, 7H), 3.06 (brs, 2H), 2.28-2.14 (m, 3H), 1.98-1.93 (m, 3H), 1.76-1.62 (m, 2H), 1.23 (brs, 1H).

LCMS=[M+H]⁺: 584.24; Purity=95.07%.

### Example 90

### Compound 940

### Preparation of 1-(4-(2,2,2-trifluoro-1-((4-(4-morpholino-1H-pyrrolo[3,2-c]pyridin-2-yl)phenyl)amino)ethyl)-[1,4′-bipiperidin]-1′-yl)prop-2-en-1-one (Compound 940)

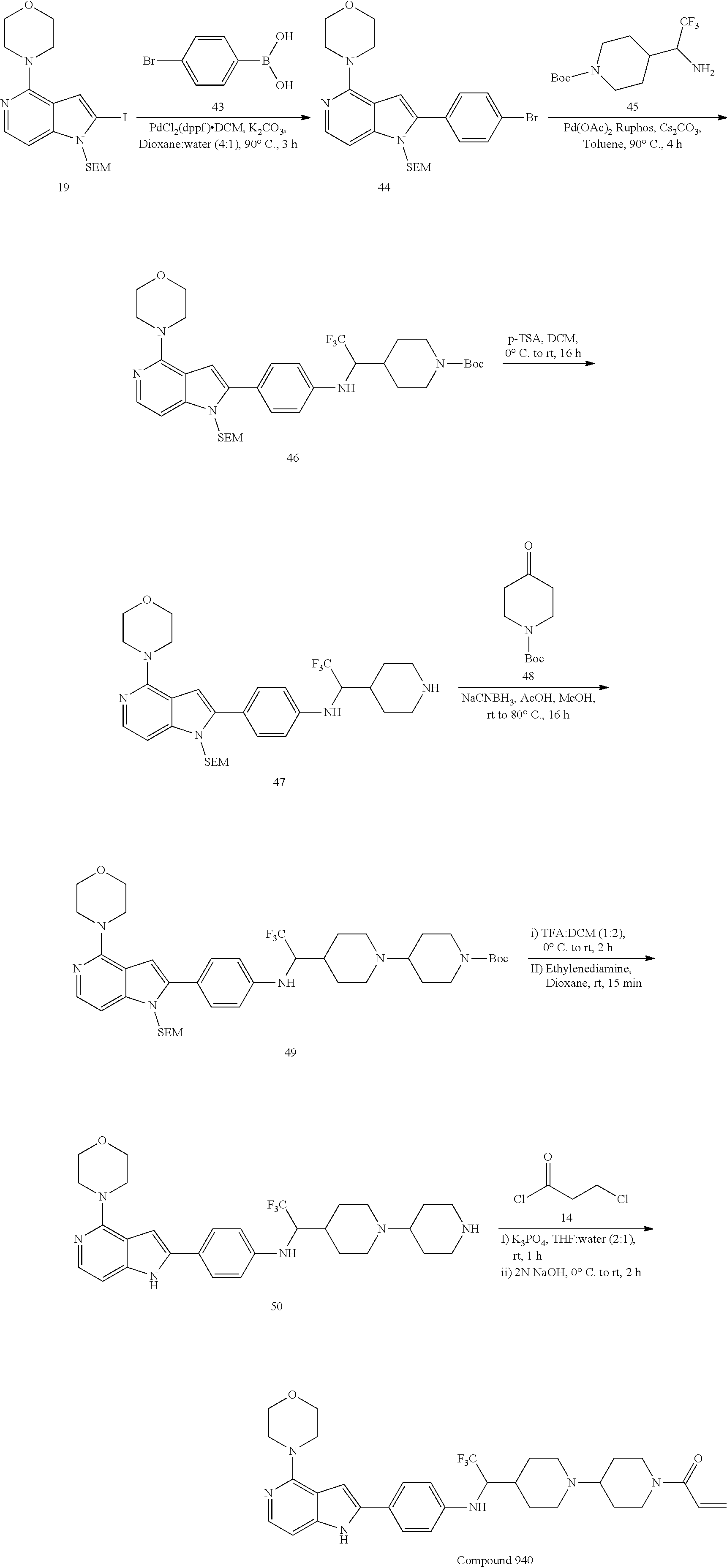

### Synthesis of 4-(2-(4-bromophenyl)-1-((2-(trimethylsilyl)ethoxy)methyl)-1H-pyrrolo[3,2-c]pyridin-4-yl)morpholine (44)

To a stirred solution of 4-(2-iodo-1-((2-(trimethylsilyl)ethoxy)methyl)-1H-pyrrolo[3,2-c]pyridin-4-yl)morpholine 19 (2 g, 4.35 mmol) and (4-bromophenyl) boronic acid 43 (699 mg, 3.48 mmol) in 1,4-dioxane (20 mL) and water (5 mL) was added K₂CO₃ (1.5 g, 10.9 mmol) at RT. The reaction mixture was degassed with N2 gas for 15 min, followed by PdCl₂(dppf)·DCM (355 mg, 0.435 mmol) was added to the reaction mixture at RT. The reaction mixture was stirred at 90° C. for 3 h. After completion of reaction (monitored by TLC and LCMS), the reaction mixture was diluted with water (50 mL) and extracted with EtOAc (3×80 mL). The combined organic layer was dried over Na₂SO₄, filtered and evaporated under reduced pressure. The crude was purified by flash column chromatography (silica gel 100-200 mesh, 12 g SNAP) using eluents 20-25% EtOAc in heptane to get the title compound as a white solid 44 (1.5 g, 46%).

LCMS=[M+H]⁺: 488.20; Purity=78%.

### Synthesis of tert-butyl 4-(2,2,2-trifluoro-1-((4-(4-morpholino-1-((2-(trimethylsilyl)ethoxy)methyl)-1H-pyrrolo[3,2-c]pyridin-2-yl)phenyl)amino)ethyl)piperidine-1-carboxylate (46)

To a stirred solution of 4-(2-(4-bromophenyl)-1-((2-(trimethylsilyl)ethoxy)methyl)-1H-pyrrolo[3,2-c]pyridin-4-yl)morpholine 44 (1.3 g, 2.66 mmol) and tert-butyl 4-(1-amino-2,2,2-trifluoroethyl)piperidine-1-carboxylate 45 (977 mg, 3.46 mmol) in toluene (15 mL) was added Cs₂CO₃ (2.6 g, 7.98 mmol) at RT. The reaction mixture was degassed with N₂ gas for 10 min, followed by RuPhos (248 mg, 0.532 mmol) and Pd (OAc) 2 (59.7 mg, 0.266 mmol) and stirred at 90° C. for 4 h. After completion of reaction (monitored by TLC and LCMS), the reaction mixture was filtered through celite pad and washed with EtOAc (100 mL). The filtrate was dilute with water (60 mL) and extracted with EtOAc (3×80 mL). The combined organic layer was dried over Na₂SO₄, filtered and concentrated under reduced pressure. The crude was purified by flash column chromatography (silica gel 100-200 M, 12 g SNAP) using eluents 30-35% EtOAc in heptane to get the title compound as a light brown solid 46 (1.2 g, 46%).

LCMS=[M+H]⁺: 690.45; Purity=70%.

### Synthesis of 4-(4-morpholino-1-((2-(trimethylsilyl)ethoxy)methyl)-1H-pyrrolo[3,2-c]pyridin-2-yl)-N-(2,2,2-trifluoro-1-(piperidin-4-yl)ethyl)aniline (47)

To a stirred solution of tert-butyl 4-(2,2,2-trifluoro-1-((4-(4-morpholino-1-((2-(trimethylsilyl)ethoxy)methyl)-1H-pyrrolo[3,2-c]pyridin-2-yl)phenyl)amino)ethyl)piperidine-1-carboxylate 46 (650 mg, 0.942 mmol) in DCM (8 mL) was added p-TSA.H₂O (717 mg, 3.77 mmol) at 0° C. and stirred at RT for 16 h. After completion of reaction (monitored by TLC and LCMS), the reaction mixture was quenched with sat. NaHCO₃ to pH~8-9 and extracted with 25% IPA in CHCl₃ (3×50 mL). The combined organic layer was dried over Na₂SO₄, filtered and evaporated under reduced pressure to get the title compound as an off white semi solid 47 (550 mg; crude) which was used in next step directly without any purification.

LCMS=[M−H]: 588.21; Purity=63%.

### Synthesis of tert-butyl 4-(2,2,2-trifluoro-1-((4-(4-morpholino-1-((2-(trimethylsilyl)ethoxy)methyl)-1H-pyrrolo[3,2-c]pyridin-2-yl)phenyl)amino)ethyl)-[1,4′-bipiperidine]-1′-carboxylate (49)

To a stirred solution of 4-(4-morpholino-1-((2-(trimethylsilyl)ethoxy)methyl)-1H-pyrrolo[3,2-c]pyridin-2-yl)-N-(2,2,2-trifluoro-1-(piperidin-4-yl)ethyl)aniline 47 (500 mg, 0.848 mmol) and tert-butyl 4 -oxopiperidine-1-carboxylate 48 (338 mg, 1.7 mmol) in MeOH (8 mL) was added AcOH (97 μL 1.7 mmol) at 0° C. under N₂ atmosphere. The reaction was stirred at RT for 1 h. Then, the reaction mixture was again cooled to 0° C. and NaCNBH₃ (101 mg, 1.7 mmol) was added to the reaction mixture and stirred at 80° C. for 16 h. After completion of reaction (monitored by TLC and LCMS), the reaction mixture was concentrated under reduced pressured, quenched with saturated NaHCO₃ (20 mL) and extracted with EtOAc (3×50 mL). The combined organic layer was dried over Na₂SO₄, filtered and concentrated under reduced pressure. The crude was purified by flash column chromatography (silica gel 100-200 M, 12 g SNAP) using eluents 5-7% MeOH in DCM to get the title compound as a white solid 49 (350 mg, 35%).

LCMS=[M+H]⁺: 773.21; Purity=70%.

### Synthesis of N-(1-([1,4′-bipiperidin]-4-yl)-2,2,2-trifluoroethyl)-4-(4-morpholino-1H-pyrrolo[3,2-c]pyridin-2-yl)aniline (50)

To a stirred solution of tert-butyl 4-(2,2,2-trifluoro-1-((4-(4-morpholino-1-((2-(trimethylsilyl)ethoxy)methyl)-1H-pyrrolo[3,2-c]pyridin-2-yl)phenyl)amino)ethyl)-[1,4′-bipiperidine]-1′-carboxylate 49 (350 mg, 0.453 mmol) in DCM (4 mL) was added TFA (2 mL) dropwise at 0° C. and stirred at RT for 2 h. After completion of reaction (monitored by TLC), the reaction mixture was concentrated under reduced pressure, triturated with Et₂O and dried under reduced pressure. The obtained crude was dissolved in 1,4-dioxane (4 mL) and ethylenediamine (45 μL, 0.679 mmol) stirred at RT for 15 min. After completion of reaction (monitored by LCMS), the reaction was poured into ice cold water (30 mL) and extracted with 25% IPA in CHCl₃ (3×50 mL). The combined organic layer was dried over Na₂SO₄, filtered and concentrated under reduced pressure to get the title compound as a brown solid 50 (180 mg; crude) which was used in next step directly without further purification.

LCMS=[M+H]⁺: 543.32; Purity=60%.

### Synthesis of 1-(4-(2,2,2-trifluoro-1-((4-(4-morpholino-1H-pyrrolo[3,2-c]pyridin-2-yl)phenyl)amino)ethyl)-[1,4′-bipiperidin]-1′-yl)prop-2-en-1-one (Compound 940)

To a stirred solution of N-(1-([1,4′-bipiperidin]-4-yl)-2,2,2-trifluoroethyl)-4-(4-morpholino-1H-pyrrolo[3,2-c]pyridin-2-yl)aniline 50 (150 mg, 0.276 mmol) in THF (4 mL) was added K₃PO₄ (117 mg, 0.553 mmol) dissolved with water (2 mL) at 0° C. and stirred for 5 min. Then a solution of 3-chloropropanoyl chloride (38.6 mg, 0.304 mmol) in THF (0.5 mL) was added dropwise and stirred at RT for 1 h. After completion of starting material (acylation reaction, monitored by TLC), 2N aq. NaOH (1.6 mL, 3.32 mmol) was added to the reaction mixture at 0° C. and stirred at RT for 2 h. After completion of reaction (complete elimination, monitored by TLC and LCMS), the reaction mixture was diluted with water (30 mL) and extracted with 2-MeTHF (3×50 mL). The combined organic layer was dried over Na₂SO₄, filtered and concentrated under reduced pressure. The crude was purified by flash column chromatography (silica gel 100-200 M, 4 g SNAP) using eluents 5-9% MeOH in DCM to get the title Compound 940 (26 mg, 15%). ¹H NMR (400 MHz, DMSO-d₆): δ 11.51 (brs, 1H), 7.69 (d, J=5.6 Hz, 1H), 7.60 (d, J=8.8 Hz, 2H), 6.88-6.81 (m, 3H), 6.80-6.74 (m, 2H), 6.08-6.03 (m, 2H), 5.63 (dd, J=10.4, 2.4 Hz, 1H), 4.43-4.40 (m, 1H), 4.27-4.25 (m, 1H), 4.06-4.03 (m, 1H), 3.78-3.76 (m, 4H), 3.53-3.51 (m, 4H), 3.01-2.95 (m, 1H), 2.89-2.83 (m, 2H), 2.60-2.54 (m, 1H), 2.48-2.43 (m, 1H), 2.13-2.07 (m, 2H), 1.75-1.65 (m, 5H), 1.48-1.39 (m, 2H), 1.29-1.22 (m, 2H).

LCMS=[M+H]⁺: 597.26; Purity=96%.

### Example 91

### Compound 941

### Preparation of 1-(3-(4-((S)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 941)

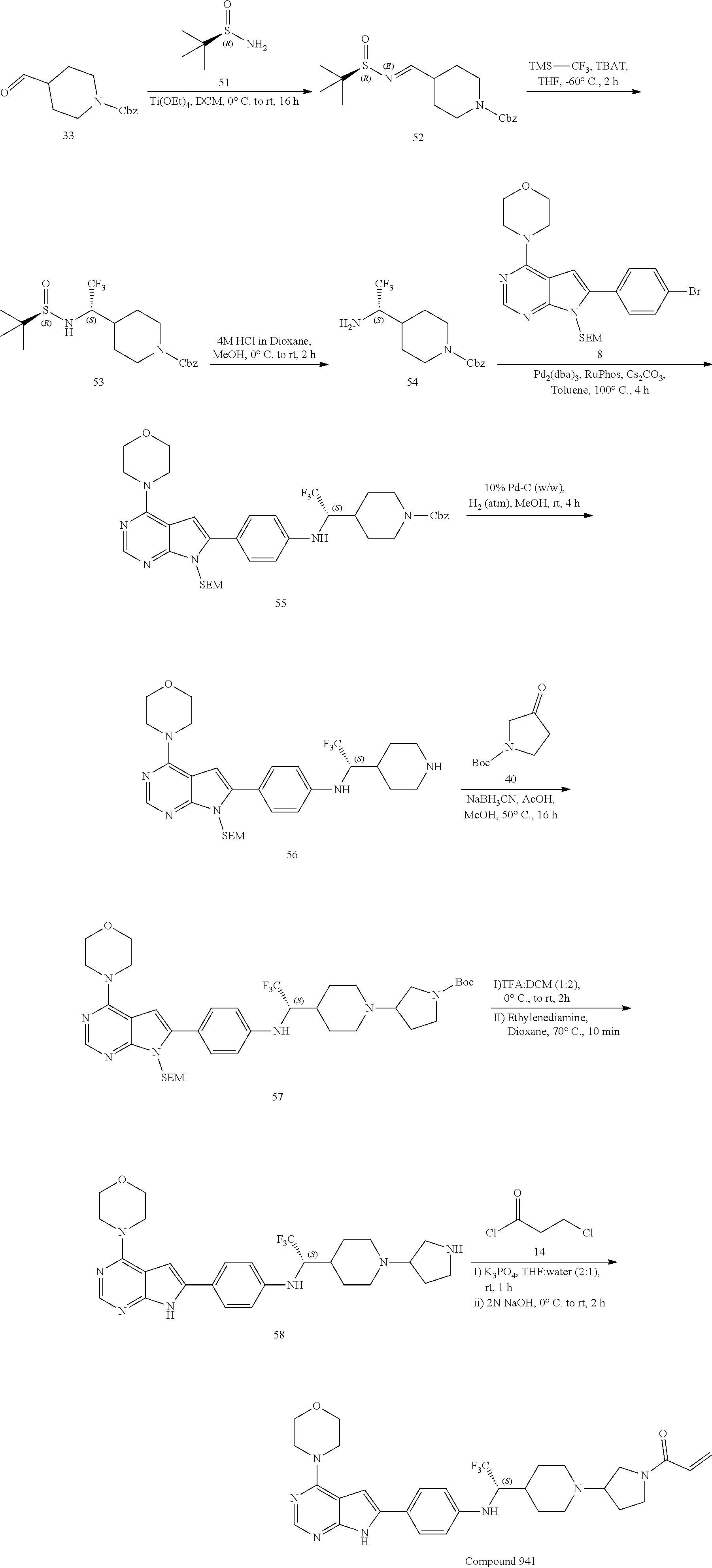

### Synthesis of benzyl (R,E)-4-(((tert-butylsulfinyl)imino)methyl)piperidine-1-carboxylate (52)

To a stirred solution of benzyl 4-formyl-1-piperidinccarboxylate 33 (20 g, 80.9 mmol) in DCM (200 mL) was added (R)-2-methylpropane-2-sulfinamide 51 (11.8 g, 97.1 mmol) at 0° C. under N₂ atmosphere, followed by Ti (OEt) 4 (34.5 mL, 162 mmol) was added slowly and stirred at RT for 16 h. After completion of reaction (monitored by TLC), the reaction mixture was quenched with brine (200 mL), filtered through celite pad and washed with DCM (200 mL). The filtrate extracted with DCM (3×150 mL). The combined organic layer was dried over Na₂SO₄, filtered and evaporated under reduced pressure. The crude was purified by flash chromatography (silica gel 100-200 M, 120 g SNAP) using eluents 25-30% EtOAc in heptane to afford the tittle compound as a colorless sticky oil 52 (22 g, 78%).

LCMS=[M+H]⁺: 351.23; Purity=88%.

### Synthesis of benzyl 4-((S)-1-(((R)-tert-butylsulfinyl)amino)-2,2,2-trifluoroethyl)piperidine-1-carboxylate (53)

To a stirred solution of benzyl (R,E)-4-(((tert-butylsulfinyl)imino)methyl)piperidine-1-carboxylate 52 (12 g, 34.2 mmol) in dry THF (100 mL) was added TBAT (20.3 g, 37.7 mmol) under N₂ atmosphere. The reaction mixture was cooled at −60° C., and a solution of TMS-CF₃ (7.6 mL, 51.4 mmol) dissolved in dry THF (10 mL) was added slowly and stirred for 2 h. After completion of reaction (monitored by TLC), the reaction mixture was quenched with ice cold water (100 mL) and extracted with EtOAc (3×100 mL). The combined organic layer was dried over Na₂SO₄, filtered and evaporated under reduced pressure. The crude was purified by flash chromatography (silica gel 100-200 M, 120 g SNAP) using eluents 25-30% EtOAc in heptane to get the title compound as a white solid 53 (10 g, 70%).

LCMS=[M+H]⁺: 421.68; Purity=70%.

### Synthesis of benzyl(S)-4-(1-amino-2,2,2-trifluoroethyl)piperidine-1-carboxylate (54)

To a stirred solution of benzyl 4-((S)-1-(((R)-tert-butylsulfinyl)amino)-2,2,2-trifluoroethyl)piperidine-1-carboxylate 53 (5 g, 11.9 mmol) in MeOH (50 mL) was added 4N HCl in 1,4-dioxane (30 mL, 119 mmol) dropwise at 0° C. and stirred at RT for 2 h. After completion of reaction (monitored by TLC), the reaction mixture was concentrated under reduced pressure. The crude was diluted with water (20 mL), basified with sat. NaHCO₃ to pH~8-9 and extracted with 25% IPA in CHCl₃ (3×100 mL). The combined organic layer was dried over Na₂SO₄, filtered and concentrated under reduced pressure to get the title compound as sticky oil 54 (3.7 g; crude) which was used in next step directly without further purification.

LCMS=[M+H]⁺: 317.19; Purity=64%.

### Synthesis of benzyl(S)-4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidine-1-carboxylate (55)

To a stirred solution of (2-{[2-(p-bromophenyl)-4-morpholino-1H-1,5,7-triazainden-1-yl]methoxy}ethyl)tris(methyl) silane 8 (1.4 g, 2.86 mmol) and benzyl 4-[(S)-1-amino-2,2,2-trifluoroethyl]-1-piperidinecarboxylate 54 (1.36 g, 4.29 mmol) in toluene (20 mL) was added Cs₂CO₃ (1.86 g, 5.72 mmol) at RT. The reaction mixture was degassed with N₂ gas for 10 min, followed by Pd₂(dba)₃ (262 mg, 0.286 mmol) and RuPhos (272 mg, 0.572 mmol) were added and stirred at 100° C. for 4 h. After completion of reaction (monitored by TLC and LCMS), the reaction mixture was diluted with water (60 mL) and extracted with EtOAc (3×100 mL). The combined organic layer was dried over Na₂SO₄, filtered and concentrated under reduced pressure. The crude was purified by flash column chromatography (silica gel 100-200 M, 40 g SNAP) using eluents 25-35% EtOAc in heptane to get the title compound as a brown semi solid 55 (1.4 g, 67%).

LCMS=[M+H]⁺: 725.33; Purity=70%.

### Synthesis of (S)-4-(4-morpholino-7-((2-(trimethylsilyl)ethoxy)methyl)-7H-pyrrolo[2,3-d]pyrimidin-6-yl)-N-(2,2,2-trifluoro-1-(piperidin-4-yl)ethyl)aniline (56)

To a stirred solution of benzyl(S)-4-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidine-1-carboxylate 55 (1.4 g, 1.93 mmol) in MeOH (20 mL) was added 10% Pd—C(1.4 g) at RT under H₂ atmosphere pressure and stirred at RT for 4 h. After completion of reaction (monitored by TLC and LCMS), the reaction mixture was filtered through celite pad and washed with MeOH (100 mL). The filtrate was concentrated under reduced pressure to get the title compound as a yellow solid 56 (1.1 g; crude) which was used in next step directly without further purification.

LCMS=[M+H]⁺: 591.27; Purity=84%.

### Synthesis of tert-butyl 3-(4-((S)-2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidin-1-yl)pyrrolidine-1-carboxylate (57)

To a stirred solution of [(S)-2,2,2-trifluoro-1-(4-piperidyl)ethyl]p-(4-morpholino-1-{[2-(trimethylsilyl) ethoxy]methyl}-1H-1,5,7-triazainden-2-yl)phenyl]amine 56 (1 g, 1.69 mmol) and tert-butyl 3-oxo-1-pyrrolidinecarboxylate 40 (470 mg, 2.54 mmol) in MeOH (20 mL) was added AcOH (0.2 mL, 3.39 mmol) at 0° C. under N₂ atmosphere and stirred at RT for 2 h. Then the reaction mixture was cooled to 0° C. and NaBH₃ CN (203 mg, 3.39 mmol) was added and stirred at 80° C. for 16 h. After completion of reaction (monitored by TLC and LCMS), the reaction mixture was evaporated under reduced pressure. The crude was basified with sat. NaHCO₃ (100 mL) and extracted with EtOAc (3×100 mL). The combined organic layer was dried over Na2SO₄, filtered and concentrated under reduced pressure. The crude was purified by flash column chromatography (silica gel 100-200 M, 40 g SNAP) using eluents 70-75% EtOAc in heptane to get the title compound as a brown semi solid 57 (700 mg, 54%).

LCMS=[M+H]⁺: 760.51; Purity=73%.

### Synthesis of 4-(4-morpholino-7H-pyrrolo[2,3-d]pyrimidin-6-yl)-N-((1S)-2,2,2-trifluoro-1-(1-(pyrrolidin-3-yl)piperidin-4-yl)ethyl)aniline (58)

To a stirred solution of tert-butyl 3-{4-[(S)-2,2,2-trifluoro-1-[p-(4-morpholino-1-{[2-(trimethylsilyl) ethoxy]methyl}-1H-1,5,7-triazainden-2-yl)phenylamino]ethyl]-1-piperidyl}-1-pyrrolidinecarboxylate 57 (600 mg, 0.789 mmol) in DCM (8 mL) was added TFA (4 mL) dropwise at 0° C. under N₂ atmosphere and stirred at RT for 2 h. After completion of reaction (monitored TLC), the reaction mixture was concentrated under reduced pressure. The crude was basified with sat. NaHCO₃ to pH~8-9 and extracted with 25% IPA in CHCl₃ (3×50 mL). The combined organic layer was dried under over Na₂SO₄, filtered and concentrated under reduced pressure to get the title compound as a brown solid 58 (500 mg; crude) which was used in next step without purification.

LCMS=[M+H]⁺: 530.25; Purity=60%.

### Synthesis of 1-(3-(4-((S)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 941)

To a stirred solution of 4-(4-morpholino-7H-pyrrolo[2,3-d]pyrimidin-6-yl)-N-((1S)-2,2,2-trifluoro-1-(1-(pyrrolidin-3-yl)piperidin-4-yl)ethyl)aniline 58 (400 mg, 0.755 mmol) in THF (6 mL) was added K₃PO₄ (321 mg, 1.51 mmol) dissolved in water (3 mL) at 0° C. under N₂ atmosphere and stirred for 5 min. Then a solution of 3-3-chloropropionyl chloride 14 (115 mg, 0.906 mmol) in THF (1 mL) was added dropwise and stirred at RT for 1 h. After completion of starting material (acylation reaction, monitored by TLC), 2N aq. NaOH (4.5 mL, 9.06 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×70 mL). The combined organic layer was dried over Na₂SO₄, filtered and concentrated under reduced pressure. The crude was purified by RP-HPLC [2-98% 0.1% TFA in Water/ACN; Column: SunFire C18 (19*250) 10 μl to get the title Compound 941 (35 mg, 8%).

¹H NMR (400 MHz, DMSO-d₆): δ 12.24 (brs, 1H), 9.40 (brs, 1H), 8.20 (s, 1H), 7.68 (d, J=8.8 Hz, 2H), 6.98-6.90 (m, 3H), 6.60-6.53 (m, 1H), 6.29 (d, J=10.8 Hz, 1H), 6.18-6.14 (m, 1H), 5.73-5.70 (m, 1H), 4.58-4.53 (m, 1H), 4.10-4.01 (m, 1H), 3.88-3.86 (m, 4H), 3.85-3.83 (m, 2H), 3.77-3.75 (m, 4H), 3.63-3.55 (m, 3H), 3.37-3.31 (m, 1H), 3.20-3.03 (m, 2H), 2.15-2.09 (m, 2H), 1.95-1.92 (m, 2H), 1.76-1.62 (m, 2H).

LCMS=[M+H]⁺: 584.17; Purity=99.85%.

### Example 92

### Compound 942 and Compound 943

### Preparation of 1-((S)-3-((R)-2,2,2-trifluoro-1-((4-(4-morpholino-7H-pyrrolo[2,3-d]pyrimidin-6-yl)phenyl)amino)ethyl)piperidin-1-yl)prop-2-en-1-one (Compound 942) and 1-((R)-3-((R)-2,2,2-trifluoro-1-((4-(4-morpholino-7H-pyrrolo[2,3-d]pyrimidin-6-yl)phenyl)amino)ethyl)piperidin-1-yl)prop-2-en-1-one (Compound 943)

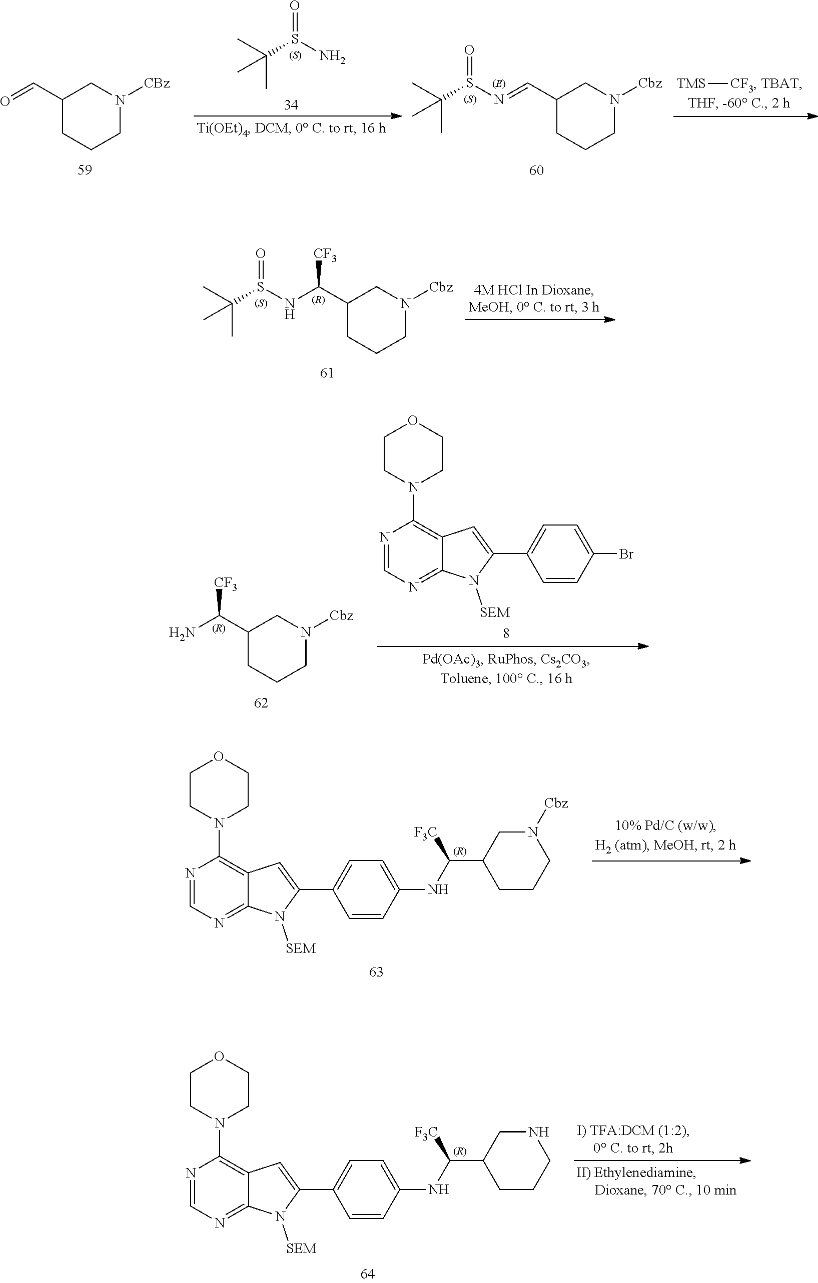

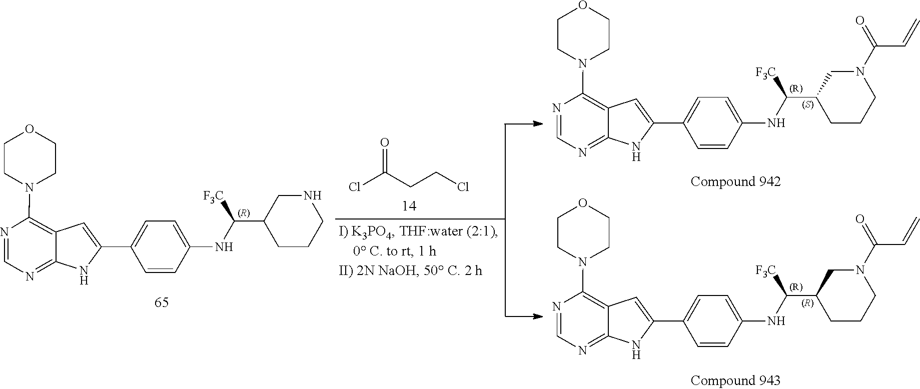

### Synthesis of benzyl 3-((E)-(((S)-tert-butylsulfinyl)imino)methyl)piperidine-1-carboxylate (60)

To a stirred solution of benzyl 3-formylpiperidine-1-carboxylate 59 (10 g, 40.4 mmol) in DCM (100 mL) was added(S)-2-methylpropane-2-sulfinamide 34 (5.88 g, 48.5 mmol) at 0° C. under N₂ atmosphere, followed by Ti (OEt)+ (17.5 mL, 80.6 mmol) was added slowly and stirred at RT for 16 h. After completion of reaction (monitored by TLC), the reaction mixture was quenched with brine (200 mL), filtered through celite pad and washed with DCM (200 mL). The filtrate extracted with DCM (3×100 mL). The combined organic layer was dried over Na₂SO₄, filtered and evaporated under reduced pressure. The crude was purified by flash chromatography (silica gel 100-200 M, 120 g SNAP) using eluents 25-30% EtOAc in heptane to afford the tittle compound as a colorless sticky oil 60 (13 g, 81%).

LCMS=[M+H]⁺: 351.17; Purity=88%.

### Synthesis of benzyl 3-((R)-1-(((S)-tert-butylsulfinyl)amino)-2,2,2-trifluoroethyl)piperidine-1-carboxylate (61)

To a stirred solution of benzyl 3-((E)-(((S)-tert-butylsulfinyl)imino)methyl)piperidine-1-carboxylate 60 (13 g, 37.1 mmol) in dry THF (100 mL) was added TBAT (22 g, 40.8 mmol) under N₂ atmosphere. The reaction mixture was cooled at −60° C., and a solution of TMS-CF₃ (11 mL, 74.2 mmol) dissolved in dry THF (10 mL) was added slowly and stirred for 2 h. After completion of reaction (monitored by TLC), the reaction mixture was quenched with ice cold water (100 mL) and extracted with EtOAc (3×100 mL). The combined organic layer was dried over Na₂SO₄, filtered and evaporated under reduced pressure. The crude was purified by flash chromatography (silica gel 100-200 M, 120 g SNAP) using eluents 25-30% EtOAc in heptane to get the title compound as a colorless sticky gel 61 (11 g, 69%).

LCMS=[M+H]⁺: 421.17; Purity=95%.

### Synthesis of benzyl 3-((R)-1-amino-2,2,2-trifluoroethyl)piperidine-1-carboxylate (62)

To a stirred solution of benzyl 3-((R)-1-(((S)-tert-butylsulfinyl)amino)-2,2,2-trifluoroethyl)piperidine-1-carboxylate 61 (5.5 g, 13.1 mmol) in MeOH (50 mL) was added 4N HCl in 1,4-dioxane (33 mL, 131 mmol) dropwise at 0° C. and stirred at RT for 2 h. After completion of reaction (monitored by TLC), the reaction mixture was concentrated under reduced pressure. The crude was diluted with water (20 mL), basified with sat. NaHCO₃ to pH~8-9 and extracted with 25% IPA in CHCl₃ (3×100 mL). The combined organic layer was dried over Na₂SO₄, filtered and concentrated under reduced pressure to get the title compound as sticky oil 62 (4.0 g; crude) which was used in next step directly without further purification.

LCMS=[M+H]⁺: 317.14; Purity=78%.

### Synthesis of benzyl 3-((R)-2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidine-1-carboxylate (63)

To a stirred solution of (2-{[2-(p-bromophenyl)-4-morpholino-1H-1,5,7-triazainden-1-yl]methoxy}ethyl)tris(methyl) silane 8 (1.4 g, 2.86 mmol) and benzyl 3-((R)-1-amino-2,2,2-trifluoroethyl)piperidine-1-carboxylate 61 (1.36 g, 4.29 mmol) in toluene (25 mL) was added Cs₂CO₃ (1.86 g, 5.72 mmol) at RT. The reaction mixture was degassed with N₂ gas for 10 min, followed by Pd₂(dba)₃ (262 mg, 0.286 mmol) and RuPhos (272 mg, 0.572 mmol) were added and stirred at 100° C. for 4 h. After completion of reaction (monitored by TLC and LCMS), the reaction mixture was diluted with water (60 mL) and extracted with EtOAc (3×100 mL). The combined organic layer was dried over Na₂SO₄, filtered and concentrated under reduced pressure. The crude was purified by flash column chromatography (silica gel 100-200 M, 40 g SNAP) using eluents 25-35% EtOAc in heptane to get the title compound as a brown semi solid 63 (1.2 g, 57%).

LCMS=[M+H]⁺: 725.34; Purity=60%.

### Synthesis of 4-(4-morpholino-7-((2-(trimethylsilyl)ethoxy)methyl)-7H-pyrrolo[2,3-d]pyrimidin-6-yl)-N-((1R)-2,2,2-trifluoro-1-(piperidin-3-yl)ethyl)aniline (64)

To a stirred solution of benzyl 3-((R)-2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidine-1-carboxylate 63 (1 g, 1.38 mmol) in MeOH (20 mL) was added 10% Pd—C(1 g) at RT under H₂ atmosphere pressure and stirred at RT for 4 h. After completion of reaction (monitored by TLC and LCMS), the reaction mixture was filtered through celite pad and washed with MeOH (100 mL). The filtrate was concentrated under reduced pressure to get the title compound as a yellow solid 64 (750 mg; crude) which was used in next step directly without further purification.

LCMS=[M+H]⁺: 591.30; Purity=92%.

### Synthesis of 4-(4-morpholino-7H-pyrrolo[2,3-d]pyrimidin-6-yl)-N-((1R)-2,2,2-trifluoro-1-(piperidin-3-yl)ethyl)aniline (65)

To a stirred solution of 4-(4-morpholino-7-((2-(trimethylsilyl)ethoxy)methyl)-7H-pyrrolo[2,3-d]pyrimidin-6-yl)-N-((1R)-2,2,2-trifluoro-1-(piperidin-3-yl)ethyl)aniline 64 (750 mg, 1.27 mmol) in DCM (8 mL) was added TFA (4 mL) dropwise at 0° C. under N₂ atmosphere and stirred at RT for 2 h. After completion of reaction (monitored TLC), the reaction mixture was concentrated under reduced pressure, triturated with Et₂O and dried under reduced pressure. The crude was dissolved in 1,4-dioxane (10 mL) and was added 1,2-ethanediamine (127 μL, 1.9 mmol) and stirred at 70° C. for 10 min. After completion of reaction (monitored by TLC), the reaction was poured into ice cold water (50 mL) and extracted with 25% IPA in CHCl₃ (3×80 mL). The combined organic layer was dried over Na₂SO₄, filtered and concentrated under reduced pressure to get the title compound as a brown solid 65 (500 mg; crude) which was used in next step directly without further purification.

LCMS=[M+H]⁺: 461.22; Purity=62%.

### Synthesis of 1-((S)-3-((R)-2,2,2-trifluoro-1-((4-(4-morpholino-7H-pyrrolo[2,3-d]pyrimidin-6-yl)phenyl)amino)ethyl)piperidin-1-yl)prop-2-en-1-one (Compound 942) and 1-((R)-3-((R)-2,2,2-trifluoro-1 -((4-(4-morpholino-7H-pyrrolo[2,3-d]pyrimidin-6-yl)phenyl)amino)ethyl)piperidin-1-yl)prop-2-en-1-one (Compound 943)

To an ice cold stirred solution of 4-(4-morpholino-7H-pyrrolo[2,3-d]pyrimidin-6-yl)-N-((IR)-2,2,2-trifluoro-1-(piperidin-3-yl)ethyl)aniline 65 (500 mg, 1.09 mmol) in THF (10 mL) was added K₃PO₄ (461 mg, 2.17 mmol) dissolved in water (5 mL) at 0° C. and stirred for 5 min. Then a solution of 3-chloropropanoyl chloride 14 (155 μL,1.63 mmol) in THF (1 mL) was added drop wise at 0° C. and stirred the reaction mixture at RT for 1 h. After completion of starting material (acylation reaction, monitored by TLC), 2N aq. NaOH (6.5 mL, 13 mmol) was added to the reaction mixture at 0° C. and stirred at 50° C. for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [5-95% 5 mM Ammonium Bicarbonate in water/ACN; Column: X Select Phenyl Hexyl (19*250) 5μ] to get the title Compound 942 (17 mg, 3%) and Compound 943 (121 mg, 21%) as a separable diastereomers.

Analytical data of Compound 942:

¹H NMR (400 MHz, DMSO-d₆): δ 11.97 (s, 1H), 8.13 (s, 1H), 7.66 (d, J=8.4 Hz, 2H), 6.89-6.87 (m, 3H), 6.83-6.76 (m, 1H), 6.15 (d, J=10.4 Hz, 1H), 6.10-6.06 (m, 1H), 5.71-5.64 (m, 1H), 4.61-4.58 (m, 1H), 4.43-4.36 (m, 1H), 4.10-3.98 (m, 1H), 3.85-3.80 (m, 4H), 3.78-3.72 (m, 4H), 2.95-2.89 (m, 1H), 1.96-1.85 (m, 2H), 1.74-1.71 (m, 1H), 1.59-1.42 (m, 1H), 1.38-1.29 (m, 1H) [1H may merged with solvent peak]LCMS=[M+H]⁺: 515.16; Purity=98.08%.

Analytical data of Compound 943:

¹H NMR (400 MHz, DMSO-d₆): δ 8.13 (s, 1H), 7.65 (d, J=8.8 Hz, 2H), 6.97-6.80 (m, 4H), 6.30-6.18 (m, 1H), 6.12-6.08 (m, 1H), 5.67-5.58 (m, 1H), 4.64-4.38 (m, 2H), 4.30-4.15 (m, 2H), 3.86-3.83 (m, 4H), 3.75-3.72 (m, 4H), 2.96-2.87 (m, 1H), 1.93-1.87 (m, 2H), 1.74-1.71 (m, 1H), 1.42-1.35 (m, 2H). [Indole NH proton is not appearing]

LCMS=[M+H]⁺: 515.16; Purity=93%.

### Example 93

### Compound 944

### Preparation of 1-(4-(2,2,2-trifluoro-1-((4-(4-morpholino-1-((2-(trimethylsilyl)ethoxy)methyl)-1H-pyrrolo[3,2-c]pyridin-2-yl)phenyl)amino)ethyl)piperidin-1-yl)prop-2-en-1-one (Compound 944)

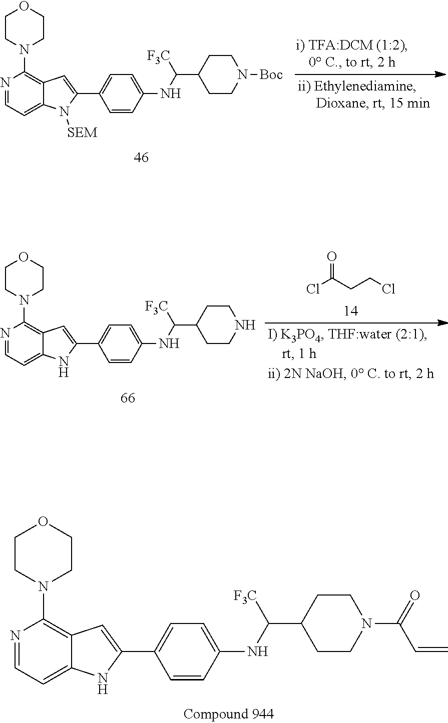 [SMILES: CC(C)(C)OC(=O)N1CCC(C(Nc2ccc(-c3cc4c(N5CCOCC5)nccc4n3COCC[Si](C)(C)C)cc2)C(F)(F)F)CC1 ; FC(F)(F)C(Nc1ccc(-c2cc3c(N4CCOCC4)nccc3[nH]2)cc1)C1CCNCC1 ; O=C(Cl)CCCl ; C=CC(=O)N1CCC(C(Nc2ccc(-c3cc4c(N5CCOCC5)nccc4[nH]3)cc2)C(F)(F)F)CC1]

### Synthesis of 4-(4-morpholino-1H-pyrrolo[3,2-c]pyridin-2-yl)-N-(2,2,2-trifluoro-1-(piperidin-4-yl)ethyl)aniline (66)

To an ice cold stirred solution of tert-butyl 4-(2,2,2-trifluoro-1-((4-(4-morpholino-1-((2-(trimethylsilyl)ethoxy)methyl)-1H-pyrrolo[3,2-c]pyridin-2-yl)phenyl)amino)ethyl)piperidine-1-carboxylate 46 (650 mg, 0.942 mmol) in DCM (8 mL) was added TFA (4 mL) dropwise at 0° C. and stirred at RT for 2 h. After completion of reaction (TLC monitoring), the reaction mixture was concentrated under reduced pressure, triturated with Et₂O and dried under reduced pressure. The obtained crude was dissolved in 1,4-dioxane (5 mL) and ethylenediamine (95 μL, 1.41 mmol) stirred at RT for 15 min. After completion of reaction (monitored by LCMS), the reaction was poured into ice cold water (30 mL) and extracted with 25% IPA in CHCl₃ (3×50 mL). The combined organic layer was dried over Na₂SO₄, filtered and concentrated under reduced pressure to get the title compound as an off white solid 66 (500 mg; crude) which was directly used in the next step without any purification.

LCMS=[M+H]⁺: 460.18; Purity=63%.

### Synthesis of 1-(4-(2,2,2-trifluoro-1-((4-(4-morpholino-1H-pyrrolo[3,2-c]pyridin-2-yl)phenyl)amino)ethyl)piperidin-1-yl)prop-2-en-1-one (Compound 944)

To a stirred solution of 4-(4-morpholino-1H-pyrrolo[3,2-c]pyridin-2-yl)-N-(2,2,2-trifluoro-1-(piperidin-4-yl)ethyl)aniline 66 (500 mg, 1.09 mmol) in THF (8 mL) was added K₃PO₄ (462 mg, 2.18 mmol) dissolved in water (4 mL) at 0° C. and stirred for 5 min. Then a solution of 3-chloropropanoyl chloride 14 (152 mg, 1.2 mmol) in THF (1 mL) was added dropwise and stirred at RT for 1 h. After completion of starting material (acylation reaction, monitored by TLC), 2N aq. NaOH (6.5 mL, 13.1 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [5-95% 0.1% TFA in water/ACN; Column: X bridge C18 (19*100) 10μ] to get the title Compound 944 (349 mg, 49%).

¹H NMR (400 MHz, DMSO-d₆): δ 12.59 (s, 1H), 7.67 (d, J=8.8 Hz, 2H), 7.60 (d, J=6.4 Hz, 1H), 7.28 (s, 1H), 7.13 (d, J=7.2 Hz, 1H), 6.93 (d, J=8.8 Hz, 2H), 6.82-6.75 (m, 1H), 6.28-6.25 (m, 1H), 6.09-6.05 (m, 1H), 5.71-5.64 (m, 1H), 4.50-4.43 (m, 2H), 4.14-4.07 (m, 1H), 3.85-3.80 (m, 8H), 3.10-3.00 (m, 1H), 2.64-2.61 (m, 1H), 2.15-2.10 (m, 1H), 1.77-1.75 (m, 2H), 1.41-1.28 (m, 2H).

LCMS=[M+H]⁺: 514.17; Purity=95.21%.

### Example 94

### Compound 945

### Preparation of 1-(4-(6-((4-(4-morpholino-7H-pyrrolo[2,3-d]pyrimidin-6-yl)phenyl)amino)pyridazin-3-yl)piperazin-1-yl)prop-2-en-1-one (Compound 945)

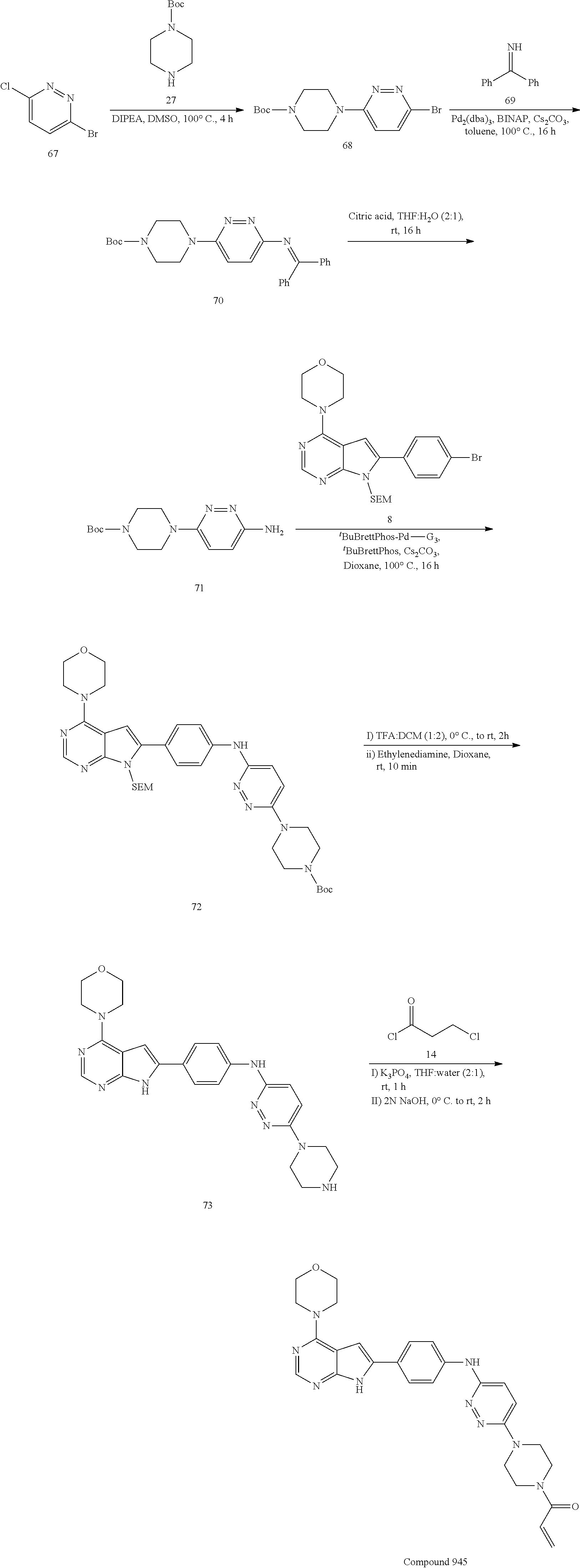

### Synthesis of tert-butyl 4-(6-bromopyridazin-3-yl)piperazine-1-carboxylate (68)

To a stirred solution of 3-bromo-6-chloropyridazine 67 (3 g, 15.5 mmol) in DMSO (30 mL) were added DIPEA (8.1 mL, 46.5 mmol) and tert-butyl piperazine-1-carboxylate 27 (4.33 g, 23.3 mmol) sequentially at RT under N₂ atmosphere and stirred at 100° C. for 4 h. After completion of reaction (monitored by TLC), the reaction mixture was cooled to RT, poured into ice cold water (100 mL) and extracted with EtOAc (3×80 mL). The combined organic layer was washed with ice cold brine (2×80 mL), dried over Na₂SO₄, filtered and evaporated under reduced pressure to get tittle compound as an off white solid 68 (4.2 g; crude), which was used in next step directly without further purification.

LCMS=[M+H]⁺: 342.98; Purity=70%

### Synthesis of tert-butyl 4-(6-((diphenylmethylene)amino)pyridazin-3-yl)piperazine-1-carboxylate (70)

To a stirred solution of tert-butyl 4-(6-bromopyridazin-3-yl)piperazine-1-carboxylate 68 (4 g, 13.4 mmol) and diphenylmethanimine 69 (3.4 mL, 20.1 mmol) in toluene (40 mL) was added Cs₂CO₃ (8.72 g, 26.8 mmol) at RT. The reaction mixture was degassed with N₂ gas for 10 min, followed by BINAP (1.67 g, 2.68 mmol) and Pd₂(dba)₃ (1.08 g, 1.34 mmol) were added and stirred at 100° C. for 16 h. After completion of reaction (monitored by TLC and LCMS), the reaction mixture was filtered through celite pad and washed with EtOAc (200 mL). The filtrate was washed with water (3×80 mL). The organic layer was separated, dried over Na₂SO₄, filtered and evaporated under reduced pressure. The crude was purified by flash column chromatography (silica gel 100-200 M, 40 g SNAP) using eluents 20-30% EtOAc in heptane to get the title compound as a light yellow solid 70 (1.9 g, 32%).

LCMS=[M+H]⁺: 444.20; Purity=75%.

### Synthesis of tert-butyl 4-(6-aminopyridazin-3-yl)piperazine-1-carboxylate (71)

To an ice cold stirred solution of tert-butyl 4-(6-((diphenylmethylene)amino)pyridazin-3-yl)piperazine-1-carboxylate 70 (1.8 g, 4.06 mmol) in THF (30 mL) was added a solution of citric acid (17.9 g, 93.3 mmol) in H₂O (15 mL) dropwise at 0° C. under N₂ atmosphere and stirred at RT for 16 h. After completion of reaction (monitored by TLC), the reaction mixture was concentrated under reduced pressure, quenched with sat. NaHCO₃ and extracted with 25% IPA in CHCl₃ (3×80 mL). The combined organic layer over dried over Na2SO₄, filtered and concentrated under reduced pressure. The crude was triturated with Et20, decanted and dried under reduced pressure to get the tittle compound as a yellow semi solid 71 (600 mg; crude) which was used in next step without purification.

LCMS=[M+H]⁺: 280.28; Purity=98%.

### Synthesis of tert-butyl 4-(6-((4-(4-morpholino-7-((2-(trimethylsilyl)ethoxy)methyl)-7H-pyrrolo[2,3-d]pyrimidin-6-yl)phenyl)amino)pyridazin-3-yl)piperazine-1-carboxylate (72)

To a stirred solution of tert-butyl 4-(6-aminopyridazin-3-yl)piperazine-1-carboxylate 71 (300 mg, 1.07 mmol) and 4-(6-(4-bromophenyl)-7-((2-(trimethylsilyl)ethoxy)methyl)-7H-pyrrolo[2,3-d]pyrimidin-4-yl)morpholine 8 (430 mg, 0.9 mmol) in 1,4-dioxane (8 mL) was added Cs₂CO₃ (583 mg, 1.79 mmol) at RT. The reaction mixture was degassed with N₂ gas for 10 min, followed by ^{t}BuBrettPhos (96.1 mg, 0.18 mmol) and ^{t}BuBrettPhos-Pd-G3 (81.2 mg, 0.9 mmol) were added and stirred at 100° C. for 16 h. After completion of reaction (monitored by TLC and LCMS), the reaction mixture was filtered through celite pad and washed with EtOAc (100 mL). The filtrate was washed with water (2×50 mL). The organic layer was separated, dried over Na2SO₄, filtered and evaporated under reduced pressure to get the title compound as a brown sticky solid 72 (620 mg; crude) which was used in next step without further purification.

LCMS=[M+H]⁺: 688.44; Purity=65%.

### Synthesis of N-(4-(4-morpholino-7H-pyrrolo[2,3-d]pyrimidin-6-yl)phenyl)-6-(piperazin-1-yl)pyridazin-3-amine (73)

To a stirred solution of tert-butyl 4-(6-((4-(4-morpholino-7-((2-(trimethylsilyl)ethoxy)methyl)-7H-pyrrolo[2,3-d]pyrimidin-6-yl)phenyl)amino)pyridazin-3-yl)piperazine-1-carboxylate 72 (600 mg, 0.87 mmol) in DCM (8 mL) was added TFA (4 mL) dropwise under N₂ atmosphere at 0° C. The reaction mixture was stirred at RT for 2 h. After completion of reaction (monitored by TLC and LCMS), the reaction mixture was concentrated under reduced pressure, triturated with Et₂O and dried under reduced pressure. The crude was dissolved in 1,4-dioxane (6 mL) and ethylenediamine (0.14 mL, 2.05 mmol) was added to the reaction mixture and stirred at RT for 10 min. After completion of reaction (monitored by LCMS), the reaction was poured into ice cold water (50 mL) and extracted with 25% IPA in CHCl₃ (3×50 mL). The combined organic layer was dried over Na₂SO₄, filtered and concentrated under reduced pressure to get the title compound as a brown solid 73 (400 mg; crude) which was used in next step directly without further purification.

LCMS=[M+H]⁺: 458.24; Purity=75%.

### Synthesis of 1-(4-(6-((4-(4-morpholino-7H-pyrrolo[2,3-d]pyrimidin-6-yl)phenyl)amino)pyridazin-3-yl)piperazin-1-yl)prop-2-en-1-one (Compound 945)

To an ice cold stirred solution of N-(4-(4-morpholino-7H-pyrrolo[2,3-d]pyrimidin-6-yl)phenyl)-6-(piperazin-1-yl)pyridazin-3-amine 73 (300 mg, 0.6 mmol) in THF (6 mL) was added K₃PO₄ (278 mg, 1.31 mmol) dissolved in H₂O (3 mL) under N₂ atmosphere and stirred for 5 min. Then added stock solution of 3-chloropropanoyl chloride 14 (0.1 mL, 0.98 mmol) in THF (1 mL) drop wise to the reaction mixture at 0° C. and stirred at RT for 1 h. After completion of starting material (acylation reaction, monitored by TLC), 2N aq. NaOH (3.5 mL, 7.2 mmol) was added to the reaction mixture at 0° C. and stirred at RT for 2 h. After completion of reaction (complete elimination, monitored by TLC and LCMS), the reaction mixture was diluted with water (30 mL) and extracted with 2-Me THF (3×50 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get the title Compound 945 (130 mg, 30%).

¹H NMR (400 MHz, DMSO-d₆): δ 12.52 (s, 1H), 9.75 (s, 1H), 8.26 (s, 1H), 7.91 (d, J=8.8 Hz, 2H), 7.85 (d, J=9.6 Hz, 1H), 7.70 (d, J=8.4 Hz, 2H), 7.48 (d, J=10.0 Hz, 1H), 7.20 (s, 1H), 6.90-6.83 (m, 1H), 6.17 (dd, J=16.4 Hz, 2 Hz, 1H), 5.74 (dd, J=10.4 Hz, 2 Hz, 1H), 3.92-3.91 (m, 4H), 3.79-3.76 (m, 8H), 3.72-3.68 (m, 4H).

LCMS=[M+H]⁺: 512.17; Purity=99.26%.

### Example 95

### Compound 946 and Compound 947

### Preparation of 1-((S)-3-((S)-2,2,2-trifluoro-1-((4-(4-morpholino-7H-pyrrolo[2,3-d]pyrimidin-6-yl)phenyl)amino)ethyl)piperidin-1-yl)prop-2-en-1-one (Compound 946) and 1-((S)-3-((R)-2,2,2-trifluoro-1-((4-(4-morpholino-7H-pyrrolo[2,3-d]pyrimidin-6-yl)phenyl)amino)ethyl)piperidin-1-yl)prop-2-en-1-one (Compound 947)

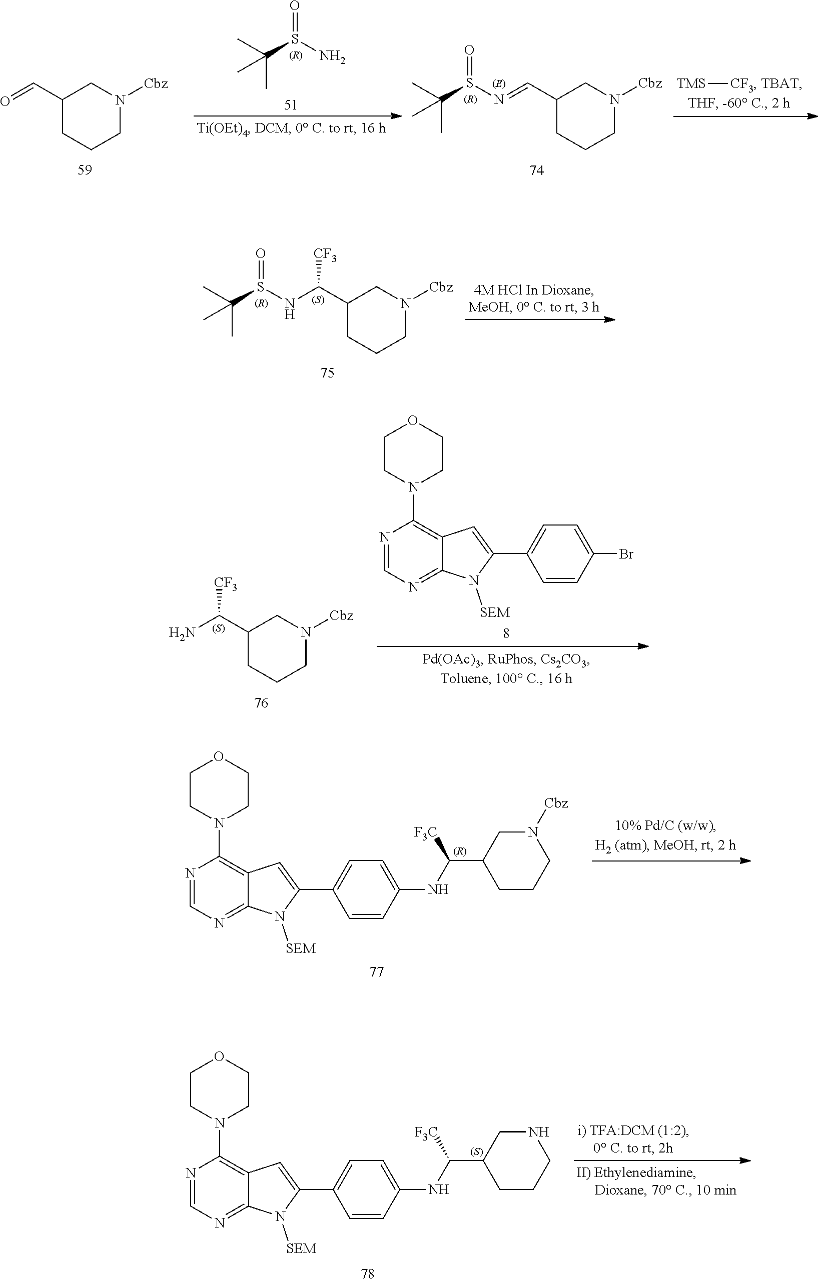

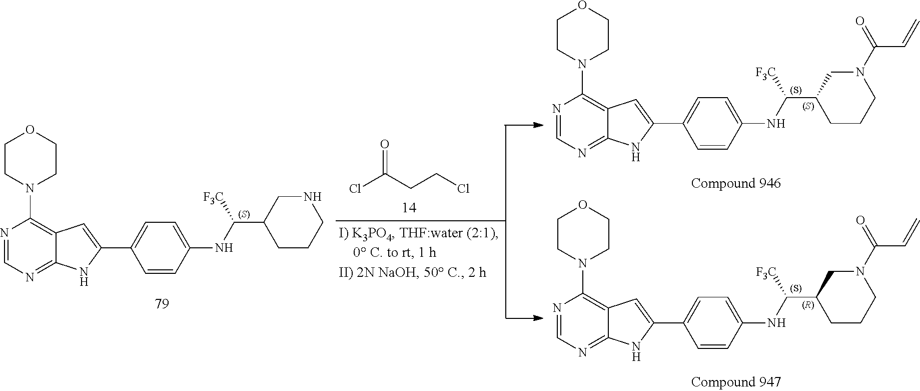

### Synthesis of benzyl 3-((E)-(((R)-tert-butylsulfinyl)imino)methyl)piperidine-1-carboxylate (74)

To a stirred solution of benzyl 3-formylpiperidine-1-carboxylate 59 (5 g, 20.2 mmol) in DCM (50 mL) was added (R)-2-methylpropane-2-sulfinamide 51 (2.94 g, 24.3 mmol) at 0° C. under N₂ atmosphere, followed by Ti (OEt) 4 (8.63 mL, 40.4 mmol) was added at 0° C. and stirred at RT for 16 h. After completion of reaction (monitored by TLC), the reaction mixture was quenched with brine (100 mL), filtered through celite pad and washed with DCM (150 mL). The filtrate extracted with DCM (3×100 mL). The combined organic layer was dried over Na₂SO₄, filtered and evaporated under reduced pressure. The crude was purified by flash chromatography (silica gel 100-200 M, 40 g SNAP) using eluents 25-30% EtOAc in heptane to get the tittle compound as a colorless sticky oil 74 (6 g, 85%) LCMS=[M+H]⁺: 351.14; Purity=98%.

### Synthesis of benzyl 3-((S)-1-(((R)-tert-butylsulfinyl)amino)-2,2,2-trifluoroethyl)piperidine-1-carboxylate (75)

To a stirred solution of benzyl 3-((E)-(((R)-tert-butylsulfinyl)imino)methyl)piperidine-1-carboxylate 74 (5 g, 14.3 mmol) in dry THF (60 mL) was added TBAT (8.47 g, 15.7 mmol) under N₂ atmosphere. The reaction mixture was cooled at −60° C., and a solution of TMS-CF₃ (3.2 mL, 21.4 mmol) dissolved in dry THF (5 mL) was added slowly and stirred for 2 h. After completion of reaction (monitored by TLC), the reaction mixture was quenched with ice cold water (100 mL) and extracted with EtOAc (3×100 mL). The combined organic layer was dried over Na₂SO₄, filtered and evaporated under reduced pressure. The crude was purified by flash chromatography (silica gel 100-200 M, 40 g SNAP) using eluents 25-30% EtOAc in heptane to get the title compound as a white solid 75 (3.5 g, 74%).

LCMS=[M+H]⁺: 421.22; Purity=97%.

### Synthesis of benzyl 3-((S)-1-amino-2,2,2-trifluoroethyl)piperidine-1-carboxylate (76)

To a stirred solution benzyl 3-((S)-1-(((R)-tert-butylsulfinyl)amino)-2,2,2-trifluoroethyl)piperidine-1-carboxylate 75 (3.5 g, 8.32 mmol) in MeOH (30 mL) was added 4N HCl in 1,4-dioxane (21 mL, 83.2 mmol) dropwise at 0° C. and stirred at RT for 2 h. After completion of reaction (monitored by TLC), the reaction mixture was concentrated under reduced pressure. The crude was diluted with water (20 mL), basified with sat. NaHCO₃to pH~8-9 and extracted with 25% IPA in CHCl₃ (3×60 mL). The combined organic layer was dried over Na2SO₄, filtered and concentrated under reduced pressure to get the title compound as sticky oil 76 (2 g; crude) which was used in next step directly without further purification.

LCMS=[M+H]⁺: 317.18; Purity=90%.

### Synthesis of benzyl 3-((R)-2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidine-1-carboxylate (77)

To a stirred solution of 4-(6-(4-bromophenyl)-7-((2-(trimethylsilyl)ethoxy)methyl)-7H-pyrrolo[2,3-d]pyrimidin-4-yl)morpholine 8 (1 g, 2.04 mmol) and benzyl 3-((S)-1-amino-2,2,2-trifluoroethyl)piperidine-1-carboxylate 76 (1.74 g, 5.52 mmol) in toluene (15 mL) was added Cs₂CO₃ (2 g, 6.13 mmol) at RT. The reaction mixture was degassed with N₂ gas for 10 min, followed by Pd (OAc) 2 (45.9 mg, 0.204 mmol) and RuPhos (191 mg, 0.409 mmol) were added and stirred at 100° C. for 16 h. After completion of reaction (monitored by TLC and LCMS), the reaction mixture was diluted with water (50 mL) and extracted with EtOAc (3×80 mL). The combined organic layer was dried over Na₂SO₄, filtered and concentrated under reduced pressure. The crude was purified by flash column chromatography (silica gel 100-200 M, 24 g SNAP) using eluents 25-35% EtOAc in heptane to get the title compound as a brown semi solid 77 (850 mg, 58%).

LCMS=[M+H]⁺: 725.43; Purity=86%.

### Synthesis of 4-(4-morpholino-7-((2-(trimethylsilyl)ethoxy)methyl)-7H-pyrrolo[2,3-d]pyrimidin-6-yl)-N-((1S)-2,2,2-trifluoro-1-(piperidin-3-yl)ethyl)aniline (78)

To a stirred solution of benzyl 3-((R)-2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)piperidine-1-carboxylate 77 (850 mg, 1.17 mmol) in MeOH (10 mL) was added 10% Pd—C(850 mg) at RT under H₂ atmosphere pressure and stirred at RT for 2 h. After completion of reaction (monitored by TLC and LCMS), the reaction mixture was filtered through celite pad and washed with MeOH (80 mL). The filtrate was concentrated under reduced pressure to get the title compound as a yellow solid 78 (600 mg; crude) which was used in next step directly without further purification.

LCMS=[M+H]⁺: 591.24; Purity=86%.

### Synthesis of 4-(4-morpholino-7H-pyrrolo[2,3-d]pyrimidin-6-yl)-N-((1S)-2,2,2-trifluoro-1-(piperidin-3-yl)ethyl)aniline (79)

To a stirred solution of 4-(4-morpholino-7-((2-(trimethylsilyl)ethoxy)methyl)-7H-pyrrolo[2,3-d]pyrimidin-6-yl)-N-((1S)-2,2,2-trifluoro-1-(piperidin-3-yl)ethyl)aniline 78 (600 mg, 1.02 mmol) in DCM (10 mL) was added TFA (5 mL) dropwise at 0° C. under N₂ atmosphere and stirred at RT for 2 h. After completion of reaction (monitored by TLC and LCMS), the reaction mixture was concentrated under reduced pressure, triturated with Et₂O and dried under reduced pressure. The obtained crude was dissolved in 1,4-dioxane (10 mL) and ethylenediamine (0.1 mL, 1.52 mmol) was added to the reaction mixture and stirred at 70° C. for 10 min. After completion of reaction (monitored by LCMS), the reaction was poured into ice cold water (50 mL) and extracted with 25% IPA in CHCl₃ (3×50 mL). The combined organic layer was dried over Na₂SO₄, filtered and concentrated under reduced pressure to get the title compound as a brown solid 79 (500 mg; crude) which was used in next step directly without further purification.

LCMS=[M+H]⁺: 461.30.40; Purity=72%.

### Synthesis of 1-((S)-3-((S)-2,2,2-trifluoro-1-((4-(4-morpholino-7H-pyrrolo[2,3-d]pyrimidin-6-yl)phenyl)amino)ethyl)piperidin-1-yl)prop-2-en-1-one (Compound 946) and 1-((R)-3-((S)-2,2,2-trifluoro-1-((4-(4-morpholino-7H-pyrrolo[2,3-d]pyrimidin-6-yl)phenyl)amino)ethyl)piperidin-1-yl)prop-2-en-1-one (Compound 947)

To a stirred solution of 4-(4-morpholino-7H-pyrrolo[2,3-d]pyrimidin-6-yl)-N-((1S)-2,2,2-trifluoro-1-(piperidin-3-yl)ethyl)aniline 79 (500 mg, 1.09 mmol) in THF (8 mL) was added K₃PO₄ (461 mg, 2.17 mmol) dissolved in water (4 mL) at 0° C. under N₂ atmosphere and stirred for 5 min. Then a solution of 3-3-chloropropionyl chloride 14 (207 mg, 1.63 mmol) in THF (1 mL) was added dropwise and stirred at RT for 1 h. After completion of starting material (acylation reaction, monitored by TLC), 2N aq. NaOH (6.5 mL, 13 mmol) was added to the reaction mixture at 0° C. and stirred at 50° C. for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [5-95% 5 mM Ammonium bicarbonate in water/ACN; Column: X Bridge C18 (19*250) 5μ] to get the title Compound 946 (34 mg, 6%) and Compound 947 (41 mg, 7%) as a separable diastereomers.

Analytical data of Compound 942:

¹H NMR (400 MHz, DMSO-d₆): δ 11.97 (brs, 1H), 8.15 (s, 1H), 7.66 (d, J=8.4 Hz, 2H), 6.89-6.76 (m, 4H), 6.15 (d, J=10.4 Hz, 1H), 6.10-6.06 (m, 1H), 5.71-5.64 (m, 1H), 4.61-4.36 (m, 2H), 4.17-3.98 (m, 1H), 3.85-3.83 (m, 4H), 3.80-3.75 (m, 4H), 2.95-2.89 (m, 1H), 2.45-2.42 (m, 1H), 1.90-1.81 (m, 2H), 1.78-1.59 (m, 1H), 1.59-1.46 (m, 1H), 1.38-1.29 (m, 1H).

LCMS=[M+H]⁺: 515.12; Purity=98.72%.

Analytical data of Compound 943:

¹H NMR (400 MHz, DMSO-d₆): δ 11.97 (brs, 1H), 8.13 (d, J=6.4 Hz, 1H), 7.66 (d, J=8.4 Hz, 2H), 6.89-6.84 (m, 3H), 6.80-6.58 (m, 1H), 6.30-6.20 (m, 1H), 6.09-5.99 (m, 1H), 5.66-5.58 (m, 1H), 4.64-4.16 (m, 2H), 4.02-3.96 (m, 1H), 3.86-3.80 (m, 4H), 3.75-3.67 (m, 4H), 3.15-2.85 (m, 1H), 2.76-2.60 (m, 1H), 1.96-1.86 (m, 2H), 1.79-1.71 (m, 1H), 1.56-1.35 (m, 2H).

LCMS=[M+H]⁺: 515.10; Purity=96.20%.

### Example 96

### Compound 948

### Preparation of 1-(4-((S)-3-((S)-2,2,2-trifluoro-1-((4-(4-morpholino-7H-pyrrolo[2,3-d]pyrimidin-6-yl)phenyl)amino)ethyl)pyrrolidin-1-yl)piperidin-1-yl)prop-2-en-1-one (Compound 948)

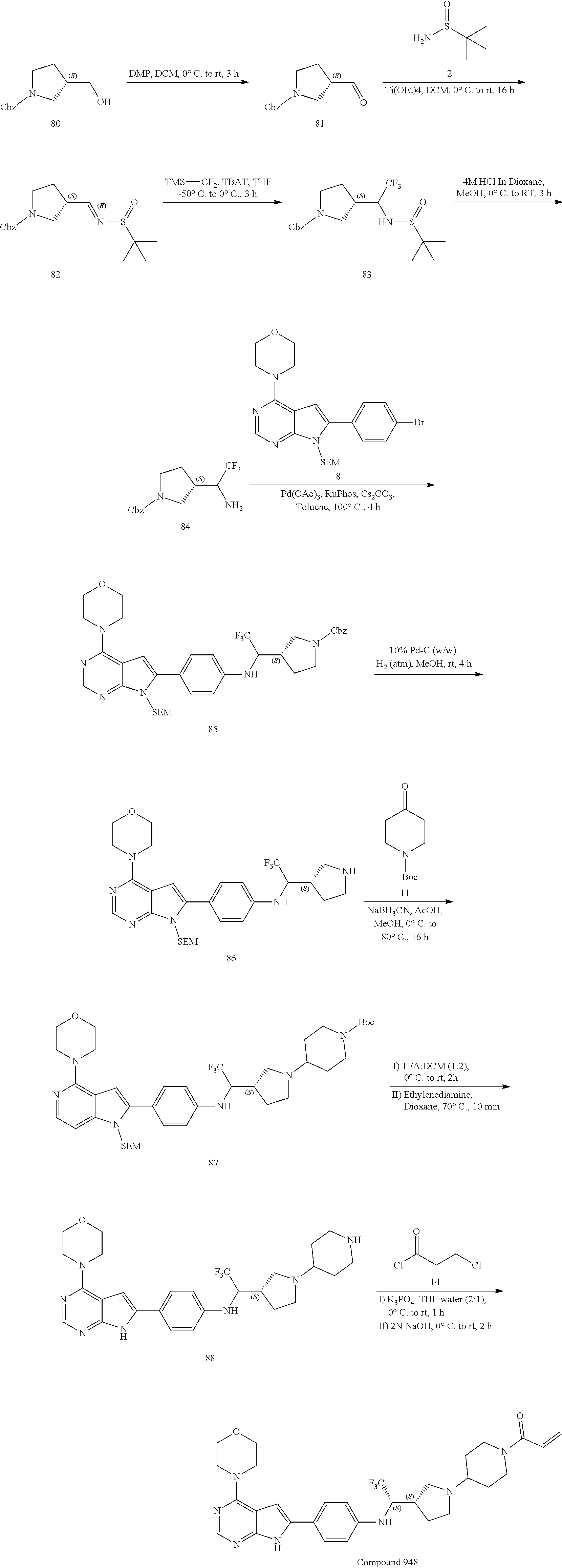

### Synthesis of Benzyl(S)-3-formylpyrrolidine-1-carboxylate (81)

To a stirred solution of benzyl(S)-3-(hydroxymethyl)pyrrolidine-1-carboxylate 80 (5 g, 21.3 mmol) in DCM (50 mL) was added DMP (10.8 g, 25.5 mmol) portion wise at 0° C. under N₂ atmosphere and stirred at RT for 3 h. After completion of reaction (monitored by TLC), the reaction mixture was quenched with sat. Na2S203 (100 mL) and sat. NaHCO₃ (100 mL) and stirred at RT for 15 min, till it became clear solution. The reaction mixture was extracted with DCM (2×100 mL). The combined organic layer was washed with sat. NaHCO₃ (2×50 mL), dried over Na₂SO₄, filtered and concentrated under reduced pressure to get the title compound as a yellow sticky gel 81 (5 g; crude) which was used in next step without further purification.

¹H NMR (400 MHz, DMSO-d₆): δ 9.61 (s, 1H), 7.35-7.25 (m, 5H), 5.06 (s, 2H), 3.66-3.63 (m, 1H), 3.47-3.40 (m, 2H), 3.27-3.18 (m, 2H), 2.14-2.11 (m, 2H).

### Synthesis of Benzyl (3S)-3-((E)-((tert-butylsulfinyl)imino)methyl)pyrrolidine-1-carboxylate (82)

To a stirred solution of benzyl(S)-3-formylpyrrolidine-1-carboxylate 81 (5 g, 21.4 mmol) in DCM (50 mL) was added 2-methylpropane-2-sulfinamide 2 (3.12 g, 25.7 mmol) at 0° C. under N₂ atmosphere, followed by Ti (OEt) 4 (9.15 mL, 42.9 mmol). The reaction mixture was stirred at RT for 16 h. After completion of reaction (monitored by TLC), the reaction mixture was quenched with brine solution (100 mL), filtered through celite pad and washed with DCM (100 mL). The filtrate was extracted with DCM (3×100 mL). The combined organic layer was dried over Na₂SO₄, filtered and evaporated under reduced pressure. The crude was purified by flash column chromatography (silica gel 100-200 M, 40 g SNAP) using eluents 30-40% EtOAc in heptane to get the title compound as a colorless sticky solid 82 (6 g, 83%).

LCMS=[M+H]⁺: 337.21; Purity=95%.

### Synthesis of Benzyl (3S)-3-(1-((tert-butylsulfinyl)amino)-2,2,2-trifluoroethyl)pyrrolidine-1-carboxylate (83)

To a stirred solution of benzyl (3S)-3-((E)-((tert-butylsulfinyl)imino)methyl)pyrrolidine-1-carboxylate 82 (5.5 g, 16.3 mmol) in dry THF (50 mL) was added TBAT (9.71 g, 18 mmol) under N₂ atmosphere. The reaction mixture was cooled at −60° C., and a solution of TMS-CF₃ (4.83 mL, 32.7 mmol) dissolved in dry THF (10 mL) was added slowly and stirred for 1 h. Then the reaction mixture was warmed to 0° C. and stirred for 3 h. After completion of reaction (monitored by TLC), the reaction mixture was quenched with ice cold water (100 mL) and extracted with EtOAc (3×100 mL). The combined organic layer was dried over Na₂SO₄, filtered and evaporated under reduced pressure. The crude was purified by flash chromatography (silica gel 100-200 M, 40 g SNAP) using eluents 25-30% EtOAc in heptane to get the title compound as colorless sticky oil 83 (5 g, 75%) as a 1:1 mixture of diastereomers.

LCMS=[M+H]⁺: 407.20; Purity=94%.

### Synthesis of Benzyl (3S)-3-(1-amino-2,2,2-trifluoroethyl)pyrrolidine-1-carboxylate (84)

To a stirred solution of benzyl (3S)-3-(1-((tert-butylsulfinyl)amino)-2,2,2-trifluoroethyl)pyrrolidine-1-carboxylate 83 (4.9 g, 12.1 mmol) in MeOH (50 mL) was added 4N HCl in 1,4-dioxane (30 mL, 121 mmol) dropwise at 0° C. under N₂ atmosphere. The reaction mixture was stirred at RT for 3 h. After completion of reaction (monitored by TLC), the reaction mixture was concentrated under reduced pressure. The crude was basified with sat. NaHCO₃ to pH~7-8 and extracted with 25% IPA in CHCl₃ (3×80 mL). The combined organic was washed with brine (2×50 mL), dried over Na₂SO₄, filtered and concentrated under reduced pressure to get the title compound as an orange sticky solid 84 (3.4 g; crude) which was used as such in next step without purification.

LCMS=[M+H]⁺: 303.16; Purity=84%.

### Synthesis of Benzyl (3S)-3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)pyrrolidine-1-carboxylate (85)

To a stirred solution of benzyl (3S)-3-(1-amino-2,2,2-trifluoroethyl)pyrrolidine-1-carboxylate 84 (926 mg, 3.06 mmol) and 4-(6-(4-bromophenyl)-7-((2-(trimethylsilyl)ethoxy)methyl)-7H-pyrrolo[2,3-d]pyrimidin-4-yl)morpholine 8 (1 g, 2.04 mmol) in toluene (10 mL) was added Cs₂CO₃ (1.33 g, 4.09 mmol) at RT. The reaction mixture was degassed with N₂ gas for 10 min, followed by RuPhos (95.3 mg, 0.204 mmol) and Pd (OAc) 2 (22.9 mg, 0.102 mmol) were added and stirred at 100° C. for 4 h. After completion of reaction (monitored by TLC and LCMS), the reaction mixture was diluted with water (50 mL) and extracted with EtOAc (3×80 mL). The combined organic layer was washed with brine (2×50 mL), dried over Na₂SO₄, filtered and concentrated under reduced pressure. The crude was purified by flash column chromatography (silica gel 100-200 M, 12 g SNAP) using eluents 30-40% EtOAc in heptane to get the title compound as a yellow sticky solid 85 (620 mg, 31%).

LCMS=[M+H]⁺: 711.29; Purity=71%.

### Synthesis of 4-(4-morpholino-7-((2-(trimethylsilyl)ethoxy)methyl)-7H-pyrrolo[2,3-d]pyrimidin-6-yl)-N-(2,2,2-trifluoro-1-((S)-pyrrolidin-3-yl)ethyl)aniline (86)

To a stirred solution of benzyl (3S)-3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)pyrrolidine-1-carboxylate 85 (620 mg, 0.872 mmol) in MeOH (8 mL) was added 10% Pd—C(650 mg) at RT under H₂ atmosphere and stirred at RT for 4 h. After completion of reaction (monitored by TLC and LCMS), the reaction mixture was filtered through celite pad and washed with MeOH (50 mL). The filtrate was concentrated under reduced pressure to get the title compound as a yellow solid 86 (400 mg; crude) which was used in next step without further purification.

LCMS=[M+H]⁺: 577.30; Purity=70%.

### Synthesis of tert-Butyl 4-((3S)-3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)pyrrolidin-1-yl)piperidine-1-carboxylate (87)

To a stirred solution of 4-(4-morpholino-7-((2-(trimethylsilyl)ethoxy)methyl)-7H-pyrrolo[2,3-d]pyrimidin-6-yl)-N-(2,2,2-trifluoro-1-((S)-pyrrolidin-3-yl)ethyl)aniline 86 (400 mg, 0.694 mmol) and tert-butyl 4-oxopiperidine-1-carboxylate 11 (207 mg, 1.04 mmol) in MeOH (8 mL) was added AcOH (79.3 μL, 1.39 mmol) at 0° C. and stirred at RT for 2 h. Then the reaction mixture was again cooled to 0° C. and NaCNBH₃ (83 mg, 1.39 mmol) was added. The reaction mixture was stirred at 80° C. for 16 h. After completion of reaction (monitored by TLC and LCMS), the reaction mixture was evaporated under reduced pressure. The crude was dissolved in 25% IPA in CHCl₃ (60 mL) and washed brine (2×50 mL). The organic layer was dried over Na2SO₄, filtered and concentrated under reduced pressure. The crude was purified by flash column chromatography (silica gel 100-200 M, 12 g SNAP) using eluents 3-5% MeOH in DCM to get the title compound as an off white solid 87 (400 mg, 52%).

LCMS=[M+H]⁺: 760.43; Purity=68%.

### Synthesis of 4-(4-morpholino-7H-pyrrolo[2,3-d]pyrimidin-6-yl)-N-(2,2,2-trifluoro-1-((S)-1-(piperidin-4-yl)pyrrolidin-3-yl)ethyl)aniline (88)

To a stirred solution of tert-Butyl 4-((3S)-3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)pyrrolidin-1-yl)piperidine-1-carboxylate 87 (400 g, 0.526 mmol) in DCM (5 mL) was added TFA (2.5 mL) dropwise at 0° C. under N₂ atmosphere and stirred at RT for 2 h. After completion of reaction (monitored by TLC), the reaction mixture was concentrated under reduced pressure, triturated with Et₂O and dried over vacuum. The crude was dissolved in 1,4-dioxane (5 mL) and 1,2-ethanediamine (105 μL 1.58 mmol) was added and was stirred at 70° C. for 10 min. After completion of reaction (monitored by LCMS), the reaction mixture was diluted with water (10 mL) and extracted with 25% IPA in CHCl₃ (3×30 mL). The combined organic layer was dried over Na2SO₄, filtered and concentrated under reduced pressure to get the title compound as a yellow solid 88 (242 mg; crude) which was used in next step directly without further purification.

LCMS=[M+H]⁺: 530.30; Purity=70%.

### Synthesis of 1-(4-((S)-3-((S)-2,2,2-trifluoro-1-((4-(4-morpholino-7H-pyrrolo[2,3-d]pyrimidin-6-yl)phenyl)amino)ethyl)pyrrolidin-1-yl)piperidin-1-yl)prop-2-en-1-one (Compound 948)

To a stirred solution of 4-(4-morpholino-7H-pyrrolo[2,3-d]pyrimidin-6-yl)-N-(2,2,2-trifluoro-1-((S)-1-(piperidin-4-yl)pyrrolidin-3-yl)ethyl)aniline 88 (242 mg, 0.457 mmol) in THF (6 mL) was added K₃PO₄ (194 mg, 0.914 mmol) dissolved in water (3 mL) at 0° C. and stirred for 5 min. Then a solution of 3-chloropropanoyl chloride 14 (69.6 mg, 0.548 mmol) in THF (1 mL) was added dropwise and stirred at RT for 1 h. After completion of starting material (acylation reaction, monitored by TLC), 2N aq. NaOH (3 mL, 5.48 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select CSH C18 (19*250) 10μ] to get the title Compound 948 (7.5 mg, 3%). [Note: Chirality arbitrarily assigned]

¹H NMR (400 MHz, DMSO-d₆): δ 11.96 (s, 1H), 8.14 (s, 1H), 7.64 (d, J=8.4 Hz, 2H), 6.88 (d, J=1.6 Hz, 1H), 6.81 (d, J=8.4 Hz, 2H), 6.78-6.72 (m, 1H), 6.19 (d, J=9.6 Hz, 1H), 6.07-6.02 (m, 1H), 5.62 (t, J=8.8 Hz, 1H), 4.33-4.31 (m, 1H), 4.19-4.16 (m, 1H), 3.95-3.90 (m, 1H), 3.85-3.80 (m, 4H), 3.75-3.72 (m, 4H), 3.10-3.07 (m, 1H), 2.88-2.76 (m, 3H), 2.38-2.30 (m, 3H), 2.22-2.20 (m, 1H), 1.92-1.89 (m, 1H), 1.80-1.77 (m, 2H), 1.70-1.67 (m, 1H), 1.23-1.21 (m, 2H).

LCMS=[M+H]⁺: 584.21; Purity=91.93%.

### Example 97

### Compound 949 and Compound 950

### Preparation of (R)-1-(4-(2,2,2-trifluoro-1-((4-(4-morpholino-1H-pyrrolo[3,2-c]pyridin-2-yl)phenyl)amino)ethyl)piperidin-1-yl)prop-2-en-1-one (Compound 949) and(S)-1-(4-(2,2,2-trifluoro-1-((4-(4-morpholino-1H-pyrrolo[3,2-c]pyridin-2-yl)phenyl)amino)ethyl)piperidin-1-yl)prop-2-en-1-one (Compound 950)

Chiral separation of Compound 944 resulted in both enantiomers (R)-1-(4-(2,2,2-trifluoro-1-((4-(4-morpholino-1H-pyrrolo[3,2-c]pyridin-2-yl)phenyl)amino)ethyl)piperidin-1-yl)prop-2-en-1-one (Compound 949) and(S)-1-(4-(2,2,2-trifluoro-1-((4-(4-morpholino-1H-pyrrolo[3,2-c]pyridin-2-yl)phenyl)amino)ethyl)piperidin-1-yl)prop-2-en-1-one (Compound 950)

Note: Chirality arbitrarily assigned

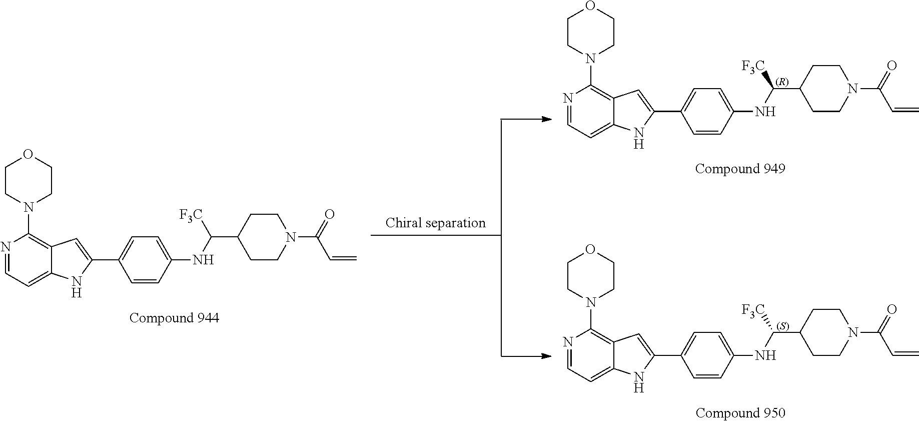

Methods for SFC purification:

Column: CHIRALCEL OJ-H (30 mm)

Mobile Phase: i) CO₂
ii) 0.2% TEA in MeOH

Flow rate: Isocratic 100 mL/Min, wavelength: 265 nm & 329 nm

Analytical data of Compound 949:

¹H NMR (400 MHz, DMSO-d₆): δ 11.51 (s, 1H), 7.69 (d, J=5.6 Hz, 1H), 7.60 (d, J=8.4 Hz, 2H), 6.88-6.75 (m, 5H), 6.10-6.05 (m, 2H), 5.68-5.63 (m, 1H), 4.50-4.47 (m, 1H), 4.43-4.37 (m, 1H), 4.14-4.07 (m, 1H), 3.78-3.76 (m, 4H), 3.54-3.51 (m, 4H), 3.09-3.00 (m, 1H), 2.63-2.57 (m, 1H), 2.15-2.02 (m, 1H), 1.77-1.74 (m, 2H), 1.42-1.29 (m, 2H).

LCMS=[M+H]⁺: 514.17; Purity=97.52%.

Analytical data of Compound 950:

¹H NMR (400 MHz, DMSO-d₆): δ 11.55 (s, 1H), 7.69 (d, J=5.6 Hz, 1H), 7.61 (d, J=8.8 Hz, 2H), 6.88-6.75 (m, 5H), 6.10-6.05 (m, 2H), 5.69-5.62 (m, 1H), 4.50-4.47 (m, 1H), 4.43-4.37 (m, 1H), 4.14-4.08 (m, 1H), 3.79-3.76 (m, 4H), 3.55-3.52 (m, 4H), 3.09-3.00 (m, 1H), 2.63-2.57 (m, 1H), 2.12-2.05 (m, 1H), 1.81-1.74 (m, 2H), 1.43-1.29 (m, 2H).

LCMS=[M+H]⁺: 514.20; Purity=99.61%

### Example 98

### Compound 951

### Preparation of 4-hydroxy-1-(4-(5-((4-(4-morpholino-7H-pyrrolo[2,3-d]pyrimidin-6 yl)phenyl)amino)pyrimidin-2-yl)piperazin-1-yl)but-2-yn-1-one (Compound 951)

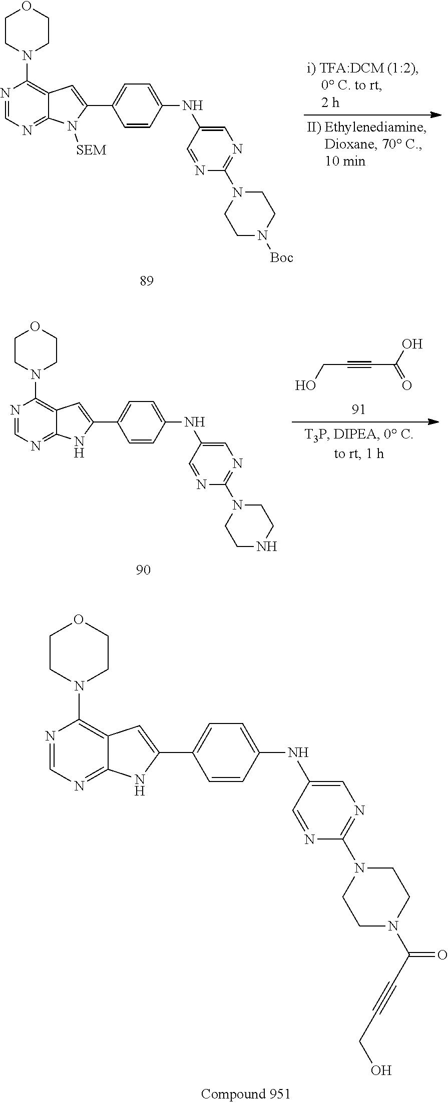 [SMILES: CC(C)(C)OC(=O)N1CCN(c2ncc(Nc3ccc(-c4cc5c(N6CCOCC6)ncnc5n4COCC[Si](C)(C)C)cc3)cn2)CC1 ; c1nc(N2CCOCC2)c2cc(-c3ccc(Nc4cnc(N5CCNCC5)nc4)cc3)[nH]c2n1 ; O=C(O)C#CCO ; O=C(C#CCO)N1CCN(c2ncc(Nc3ccc(-c4cc5c(N6CCOCC6)ncnc5[nH]4)cc3)cn2)CC1]

### Synthesis of N-(4-(4-morpholino-7H-pyrrolo[2,3-d]pyrimidin-6-yl)phenyl)-2-(piperazin-1-yl)pyrimidin-5-amine (90)

To a stirred solution of tert-butyl 4-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperazine-1-carboxylate 89 (1 g, 1.45 mmol) in DCM (20 mL) was added TFA (10 mL) drop wise at 0° C. under N₂ atmosphere. The reaction mixture was stirred at RT for 2 h. After completion of reaction (monitored by TLC), the reaction mixture was concentrated under reduced pressure, triturated with Et₂O and dried over vacuum. The crude was dissolved in 1,4-dioxane (10 mL) and 1,2-ethanediamine (146 μL 2.18 mmol) was added and was stirred at 70° C. for 10 min. After completion of reaction (monitored by LCMS), the reaction mixture was diluted with water (20 mL) and extracted with 25% IPA in CHCl₃ (3×50 mL). The combined organic layer was dried over Na₂SO₄, filtered and concentrated under reduced pressure to get the title compound as a yellow solid 90 (600 mg; crude) which was used in next step directly without further purification.

LCMS=[M+H]⁺: 458.20; Purity=76%.

### Synthesis of 4-hydroxy-1-(4-(5-((4-(4-morpholino-7H-pyrrolo[2,3-d]pyrimidin-6-yl)phenyl)amino)pyrimidin-2-yl)piperazin-1-yl)but-2-yn-1-one (Compound 951)

To a stirred solution of N-(4-(4-morpholino-7H-pyrrolo[2,3-d]pyrimidin-6-yl)phenyl)-2-(piperazin-1-yl)pyrimidin-5-amine 90 (600 mg, 1.13 mmol) in DMF (10 mL) were added DIPEA (0.7 mL, 3.93 mmol), 4-hydroxybut-2-ynoic acid 91 (131 mg, 1.13 mmol) and T3P (1.0 mL, 1.31 mmol) sequentially at 0° C. under N₂ atmosphere. The reaction mixture was stirred at RT for 1 h. After completion of reaction (monitored by TLC), the reaction mixture was poured into ice cold water (50 mL) and extracted with EtOAc (3×50 mL). The combined organic layer was washed with ice cold brine (2×50 mL), dried over Na₂SO₄, filtered and concentrated under reduced pressure to form precipitate. The crude was purified by RP-HPLC [5-95% 5 mM Ammonium Acetate in Water/ACN; Column: X Bridge C18 (19*250) 10μ] to get the title Compound 951 (19 mg, 2.7%).

¹H NMR (400 MHz, DMSO-d₆): δ 12.03 (s, 1H), 8.34 (s, 2H), 8.14 (s, 1H), 7.97 (s, 1H), 7.71 (d, J=8.0 Hz, 2H), 6.92 (s, 1H), 6.83 (d, J=8.8 Hz, 2H), 5.52 (brs, 1H), 4.29 (s, 2H) 3.85-3.83 (m, 4H), 3.78 (s, 4H), 3.74-3.70 (m, 6H), 3.60-3.58 (m, 2H).

LCMS=[M+H]⁺: 540.15; Purity=98.02%.

### Example 172

### Compound 952

### Preparation of 1-((S)-3-((R)-2,2,2-trifluoro-1-((4-(4-morpholino-7H-pyrrolo[2,3-d]pyrimidin-6-yl)phenyl)amino)ethyl)pyrrolidin-1-yl)prop-2-en-1-one (Compound 952)

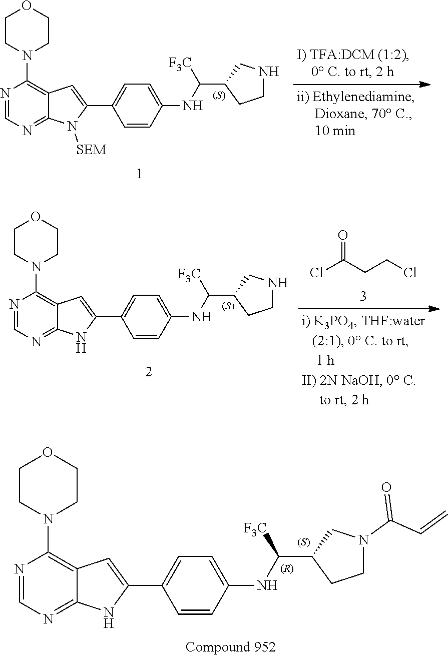

### Synthesis of 4-(4-morpholino-7H-pyrrolo[2,3-d]pyrimidin-6-yl)-N-(2,2,2-trifluoro-1-((S)-pyrrolidin-3-yl)ethyl)aniline (2)

To a stirred solution of {1-[(S)-3-pyrrolidinyl]-2,2,2-trifluoroethyl}[p-(4-morpholino-1-{[2-(trimethylsilyl) ethoxy]methyl}-1H-1,5,7-triazainden-2-yl)phenyl]amine 1 (780 mg, 1.35 mmol) in DCM (8 mL) was added TFA (4 mL) dropwise at 0° C. under N₂ atmosphere and stirred at RT for 2 h. After completion of reaction (monitored TLC), the reaction mixture was concentrated under reduced pressure, triturated with Et₂O and dried under reduced pressure. The crude was dissolved in 1,4-dioxane (6 mL) and was added 1,2-ethanediamine (0.3 mL, 4.06 mmol) at RT and stirred at 70° C. for 10 min. After completion of reaction (monitored by TLC), the reaction was poured into ice cold water (50 mL) and extracted with 25% IPA in CHCl₃ (3×80 mL). The combined organic layer was dried over Na₂SO₄, filtered and concentrated under reduced pressure to get the title compound as a brown solid 2 (450 mg; crude) which was used in next step directly without further purification.

LCMS=[M−H]: 444.18; Purity=58%.

### Synthesis of 1-((S)-3-((R)-2,2,2-trifluoro-1-((4-(4-morpholino-7H-pyrrolo[2,3-d]pyrimidin-6-yl)phenyl)amino)ethyl)pyrrolidin-1-yl)prop-2-en-1-one (Compound 952)

To an ice cold stirred solution of 4-(4-morpholino-7H-pyrrolo[2,3-d]pyrimidin-6-yl)-N-(2,2,2-trifluoro-1-((S)-pyrrolidin-3-yl)ethyl)aniline 2 (450 mg, 1.01 mmol) in THF (6 mL) was added K₃PO₄ (428 mg, 2.02 mmol) dissolved in water (5 mL) at 0° C. and stirred for 5 min. Then a solution of 3-chloropropanoyl chloride 3 (115 μL, 1.21 mmol) in THF (1 mL) was added drop wise at 0° C. and stirred at RT for 1 h. After completion of starting material (acylation reaction, monitored by TLC), 2N aq. NaOH (6.5 mL, 13 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [2-98% 0.1% TFA in water/ACN; Column: X Select CSH C18 (19*250) 10μ] to get the title Compound 952 (121 mg, 21%) as a separable diastereomer. [Note: Chirality arbitrarily assigned]

¹H NMR (400 MHz, DMSO-d₆): δ 12.48 (s, 1H), 8.25 (m, 1H), 7.69 (d, J=8.4 Hz, 2H), 7.07 (brs, 1H), 6.87 (d, J=8.4 Hz, 2H), 6.59-6.46 (m, 1H), 6.38 (t, J=9.6 Hz, 1H), 6.14-6.08 (m, 1H), 5.67-5.61 (m, 1H), 4.60-4.52 (m, 1H), 3.91-3.88 (m, 4H), 3.81-3.78 (m, 4H), 3.75-3.69 (m, 1H), 3.55-3.47 (m, 1H), 3.38 (t, J=10.4 Hz, 1H), 3.32-3.26 (m, 1H), 3.14 (t, J=11.2 Hz, 1H), 2.11-2.01 (m, 1H), 1.92-1.75 (m, 1H).

LCMS=[M+H]⁺: 501.13; Purity=98.58%.

### Example 99

### Compound 953

### Preparation of N-((R)-1-((2-(2-(4-(4-((1R,5S)-3-oxa-8-azabicyclo[3.2.1]octan-8-yl)-7H-pyrrolo[2,3-d]pyrimidin-6-yl)phenyl)-1H-imidazol-5-yl)pyridin-4-yl)methyl)piperidin-3-yl)acrylamide (Compound 953)

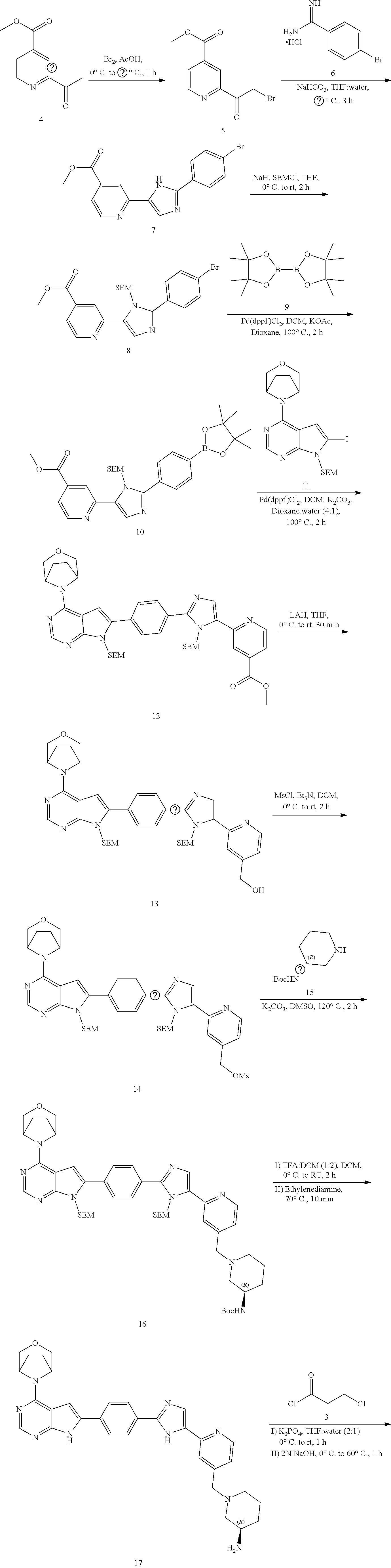

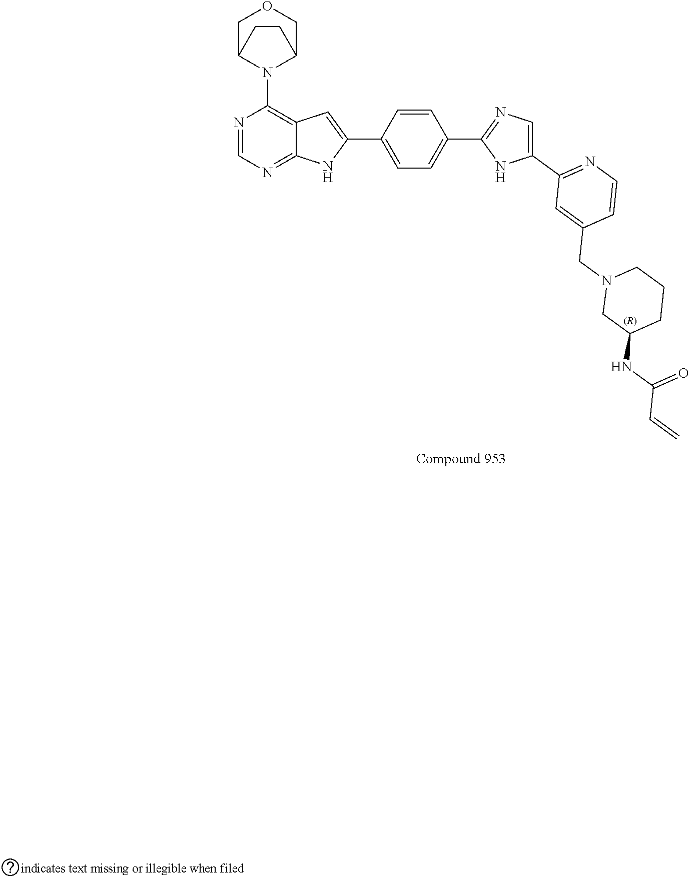

### Synthesis of methyl 2-(2-bromoacetyl)isonicotinate (5)

To a stirred solution of methyl 2-acetylisoindoline 4 (10 g, 55.8 mmol) in AcOH (40 mL) was added Br₂ (2.9 mL, 55.8 mmol) at 0° C. under N₂ atmosphere. The reaction mixture was stirred at 60° C. for 1 h. After completion the reaction (monitored by TLC), the reaction mixture poured into ice water (100 mL) and extracted with EtOAc (3×100 mL). The combined organic layer was dried over Na₂SO₄, filtered and concentrated under reduced pressure to get title compound as brown liquid 5 (11 g; crude), which was used in next step without further purification.

LCMS=[M+H]⁺: 257.97; Purity=71%.

### Synthesis of methyl 2-(2-(4-bromophenyl)-1H-imidazol-5-yl)isonicotinate (7)

To a stirred solution of 4-bromobenzimidamide hydrochloride 6 (5 g, 21.2 mmol) in THF (50 mL) and water (15 mL) was added NaHCO₃ (3.6 g, 42.5 mmol) at RT under N₂ atmosphere and stirred at 80° C. for 20 min. Then a solution of methyl 2-(2-bromoacetyl)isonicotinate 5 (11 g, 42.5 mmol) in THF (30 mL) was added slowly at 80° C. and stirred for 3 h. After completion of reaction (monitored by TLC), the reaction mixture was poured into ice cooled water (100 mL) and extracted with EtOAc (3×100 mL). The combined organic layer was washed with brine solution (2×100 mL), dried over anhydrous Na₂SO₄, filtered and concentrated under reduced pressure to get title compound as brown sticky liquid 7 (2.5 g; crude), which was used in next step without further purification.

LCMS=[M+H]⁺: 358.07; Purity=58%.

### Synthesis of methyl 2-(2-(4-bromophenyl)-1-((2-(trimethylsilyl)ethoxy)methyl)-1H-imidazol-5-yl)isonicotinate (8)

To a stirred solution of methyl 2-(2-(4-bromophenyl)-1H-imidazol-5-yl)isonicotinate 7 (1.2 g, 3.1 mmol) in THF (20 mL) was added NaH (335 mg, 8.38 mmol; 60% dispersion in mineral oil) at 0° C. under N₂ atmosphere and stirred for 10 min. Then SEMCl (1.1 mL, 6.14 mmol) was added to the reaction mixture and stirred at RT for 2 h. After completion of reaction (monitored by TLC and LCMS), the reaction mixture was quenched with ice cold water (50 mL) and extracted with EtOAc (3×50 mL). The combined organic layer dried over Na₂SO₄, filtered and concentrated under reduced pressure. The crude was purified by flash chromatography (silica gel 100-200 M, 12 g SNAP) using eluents 40-50% EtOAc in heptane to get the title compound as brown viscous liquid 8 (1.5 g, 55%).

LCMS=[M+H]⁺: 490.07; Purity=75%

### Synthesis of methyl 2-(2-(4-(4,4,5,5-tetramethyl-1,3,2-dioxaborolan-2-yl)phenyl)-1-((2-(trimethylsilyl)ethoxy)methyl)-1H-imidazol-5-yl)isonicotinate (10)

To a stirred solution of methyl 2-(2-(4-bromophenyl)-1-((2-(trimethylsilyl)ethoxy)methyl)-1H-imidazol-5-yl)isonicotinate 8 (1.2 g, 2.46 mmol) in 1,4-dioxane (15 mL) was added KOAc (723 mg, 7.37 mmol) and B₂ Pin₂ 9 (936 mg, 3.69 mmol) at RT under N₂ atmosphere. The reaction mixture was degassed with N₂ gas for 15 min, followed by PdCl₂(dppf)·DCM (200 mg, 0.246 mmol) was added and stirred at 100° C. for 2 h. After completion of reaction (monitored by TLC and LCMS), the reaction mixture was filtered through celite pad and washed with EtOAc (100 mL). The filtrate was evaporated under reduced pressure to get the title compound as a brown sticky solid 10 (2 g; crude), which was used in next step without further purification.

LCMS=[M+H]⁺: 536.33; Purity=50%.

### Synthesis of methyl 2-(2-(4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)phenyl)-1-((2-(trimethylsilyl)ethoxy)methyl)-1H-imidazol-5-yl)isonicotinate (12)

To a stirred solution of methyl 2-(2-(4-(4,4,5,5-tetramethyl-1,3,2-dioxaborolan-2-yl)phenyl)-1-((2-(trimethylsilyl)ethoxy)methyl)-1H-imidazol-5-yl)isonicotinate 10 (1.98 g, 3.7 mmol) and (1R,5S)-8-(6-iodo-7-((2-(trimethylsilyl)ethoxy)methyl)-7H-pyrrolo[2,3-d]pyrimidin-4-yl)-3-oxa-8-azabicyclo[3.2.1]octane 11 (1.2 g, 2.47 mmol) in 1,4-dioxane (40 mL) was added a solution of K₂CO₃ (784 mg, 7.4 mmol) in water (10 mL) at RT. The reaction mixture was degassed with N₂ gas for 15 min, followed by PdCl₂(dppf)·DCM (201 mg, 0.247 mmol) was added and stirred at 100° C. for 2 h. After the completion of reaction (monitored by TLC and LCMS), the reaction mixture was filtered through celite pad and washed with EtOAc (100 mL). The filtrate was diluted with water (50 mL) and extracted with EtOAc (3×80 mL). The combined organic layer was dried over Na₂SO₄, filtered and evaporated under reduced pressure. The crude was purified by flash column chromatography (silica gel 100-200 M, 24 g SNAP) using eluents 30-35% EtOAc in heptane to get the title compound as a brown solid 12 (600 mg, 24%).

LCMS=[M+H]⁺: 768.43; Purity=73%

### Synthesis of (2-(2-(4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)phenyl)-1-((2-(trimethylsilyl)ethoxy)methyl)-1H-imidazol-5-yl)pyridin-4-yl)methanol (13)

To a stirred solution of methyl 2-(2-(4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)phenyl)-1-((2-(trimethylsilyl)ethoxy)methyl)-1H-imidazol-5-yl)isonicotinate 12 (600 mg, 0.781 mmol) in dry THF (10 mL) was added LiAlH₄ (0.8 mL, 1.63 mmol; 2.5 M in THF) dropwise at 0° C. under N₂ atmosphere. The reaction mixture was stirred at RT for 30 min. After completion the reaction (monitored by TLC), the reaction mixture was cooled to 0° C. and quenched with sat. NH₄Cl and extracted with EtOAc (3×80 mL). The combined organic layer was dried over Na₂SO₄, filtered and concentrated under reduced pressure to get the title compound as brown liquid 13 (500 mg; crude), which was used in next step without further purification.

LCMS=[M+H]⁺: 740.47; Purity=50%

### Synthesis of (2-(2-(4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)phenyl)-1-((2-(trimethylsilyl)ethoxy)methyl)-1H-imidazol-5-yl)pyridin-4-yl)methyl methanesulfonate (14)

To a stirred solution of (2-(2-(4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)phenyl)-1-((2-(trimethylsilyl)ethoxy)methyl)-1H-imidazol-5-yl)pyridin-4-yl)methanol 13 (500 mg, 0.676 mmol) in DCM (8 mL) was added Et₃N (187 μL, 1.35 mmol) at 0° C. under N₂ atmosphere and stirred for 5 min. Then MsCl (53 μL, 0.676 mmol) was added at 0° C. and stirred at RT for 2 h. After completion of reaction (monitored by TLC), the reaction mixture was quenched with sat. NaHCO₃ (50 mL) and extracted with DCM (3×50 mL). The combined organic layer was dried over Na₂SO₄, filtered and concentrated under reduced pressure to get the title compound as yellow semisolid 14 (700 mg; crude), which was used in the next step directly without further purification.

LCMS=[M+H]⁺: 818.36; Purity=52%

### Synthesis of tert-butyl ((R)-1-((2-(2-(4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)phenyl)-1-((2-(trimethylsilyl)ethoxy)methyl)-1H-imidazol-5-yl)pyridin-4-yl)methyl)piperidin-3-yl)carbamate (16)

To a stirred solution of (2-(2-(4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)phenyl)-1-((2-(trimethylsilyl)ethoxy)methyl)-1H-imidazol-5-yl)pyridin-4-yl)methyl methanesulfonate 14 (700 mg, 0.856 mmol) and tert-butyl (R)-piperidin-3-ylcarbamate 15 (343 mg, 1.71 mmol) in DMSO (10 mL) was added K₂CO₃ (591 mg, 4.28 mmol) at RT under N₂ atmosphere and stirred at 120° C. for 2 h. After completion of reaction (mentored by TLC), the reaction mixture was cooled to RT and poured into ice cold water (50 mL) and extracted with EtOAc (3×80 mL). The combined organic layer was washed with ice cold brine (2×50 mL), dried over Na2SO₄, filtered and concentrated under reduced pressure. The crude was purified by flash column chromatography (silica gel 100-200 M, 12 g SNAP) using eluents 40-50% EtOAc in heptane to get the title compound as a white solid 16 (700 mg, 89%).

LCMS=[M+H]⁺: 922.62; Purity=60%

### Synthesis of (R)-1-((2-(2-(4-(4-((1R,5S)-3-oxa-8-azabicyclo[3.2.1]octan-8-yl)-7H-pyrrolo[2,3-d]pyrimidin-6-yl)phenyl)-1H-imidazol-5-yl)pyridin-4-yl)methyl)piperidin-3-amine (17)

To a stirred solution of tert-butyl ((R)-1-((2-(2-(4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)phenyl)-1-((2-(trimethylsilyl)ethoxy)methyl)-1H-imidazol-5-yl)pyridin-4-yl)methyl)piperidin-3-yl)carbamate 16 (700 mg, 0.760 mmol) in DCM (10 mL) was added TFA (5 mL) at 0° C. under N₂ atmosphere and stirred at RT for 2 h. After completion of reaction (monitored by TLC and LCMS), the reaction mixture was concentrated under reduced pressure. The crude was dissolved in 1,4-dioxane (10 mL) and 1,2-ethanediamine (110 μL, 1.63 mmol) was added and stirred at 70° C. for 10 min. After completion of reaction (monitored by LCMS), the reaction mixture was diluted with water (50 mL) and extracted with 25% IPA in CHCl₃ (3×50 mL). The combined organic layer was dried over Na₂SO₄, filtered and concentrated under reduced pressure to get the title compound as a yellow solid 17 (520 mg; crude), which was used in the next step directly without further purification.

LCMS=[M+H]⁺: 562.37; Purity=60%.

### Synthesis of N-((R)-1-((2-(2-(4-(4-((1R,5S)-3-oxa-8-azabicyclo[3.2.1]octan-8-yl)-7H-pyrrolo[2,3-d]pyrimidin-6-yl)phenyl)-1H-imidazol-5-yl)pyridin-4-yl)methyl)piperidin-3-yl)acrylamide (Compound 953)

To a stirred solution of (R)-1-((2-(2-(4-(4-((1R,5S)-3-oxa-8-azabicyclo[3.2.1]octan-8-yl)-7H-pyrrolo[2,3-d]pyrimidin-6-yl)phenyl)-1H-imidazol-5-yl)pyridin-4-yl)methyl)piperidin-3-amine 17 (500 mg, 0.890 mmol) in THF (8 mL) was added K₃PO₄ (378 mg, 1.07 mmol) dissolved in water (4 mL) at 0° C. stirred for 5 min. Then a solution of 3-chloropropionyl chloride 3 (102 μL, 1.27 mmol) in THF (1 mL) was added dropwise and stirred at RT for 1 h. After completion of starting material (acylation reaction, monitored by TLC), 2N aq. NaOH (6 mL, 11.5 mmol) was added to the reaction mixture at 0° C. and stirred at 60° C. for 1 h. After completion of reaction (complete elimination, monitored by TLC and LCMS), the reaction mixture was diluted with water (50 mL) and extracted with 2-MeTHF (3×80 mL). The combined organic layer was dried over Na2SO₄, filtered and concentrated under reduced pressure. The crude was purified by RP-HPLC [2-98% 0.1% TFA in water/ACN; Column: X Select Phenyl Hexyl (19*250) 5μ] to get the title Compound 953 (25 mg, 4%).

¹H NMR (400 MHz, DMSO-d₆): δ 12.77 (brs, 1H), 10.00 (brs, 1H), 8.67 (d, J=4.8 Hz, 1H), 8.30 (s, 2H), 8.16-8.08 (m, 5H), 7.45-7.40 (m, 2H), 6.23-6.08 (m, 2H), 5.65-5.62 (m, 1H), 5.06-5.01 (m, 2H), 4.46-4.06 (m, 6H), 3.73 (q, J=10.8 Hz, 5H), 3.47-3.39 (m, 2H), 2.96-2.72 (m, 1H), 2.11-2.06 (m, 4H), 1.91-1.87 (m, 2H), 1.73-1.70 (m, 1H), 1.44-1.41 (m, 1H).

LCMS=[M+H]⁺: 616.22; Purity=99.50%.

### Synthesis of 4-bromobenzimidamide hydrochloride (6)

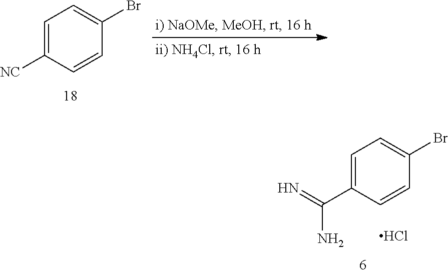 [SMILES: N#Cc1ccc(Br)cc1 ; N=C(N)c1ccc(Br)cc1 ; Cl]

To a stirred solution of 4-bromobenzonitrile 18 (5 g, 27.5 mmol) in MeOH (50 mL) was added NaOMe (2.23 g, 41.2 mmol) at RT under N₂ atmosphere and stirred at RT for 16 h. After completion of starting material (monitored by TLC), solid NH₄Cl (3 g, 54.9 mmol) was added to the reaction mixture and stirred at RT for 16 h. After completion of reaction (monitored by TLC), the reaction mixture was concentrated under reduced pressure to get the title compound as a white solid 6 (7 g; crude as HCl salt), which was used in next step directly without further purification.

LCMS=[M+H]⁺: 199.90; Purity=97%

### Example 100

### Compound 954

### Preparation of (R)—N-(1-(6-((4-(4-morpholino-7H-pyrrolo[2,3-d]pyrimidin-6-yl)phenyl)amino)pyridazin-3-yl)piperidin-3-yl)acrylamide (Compound 954)

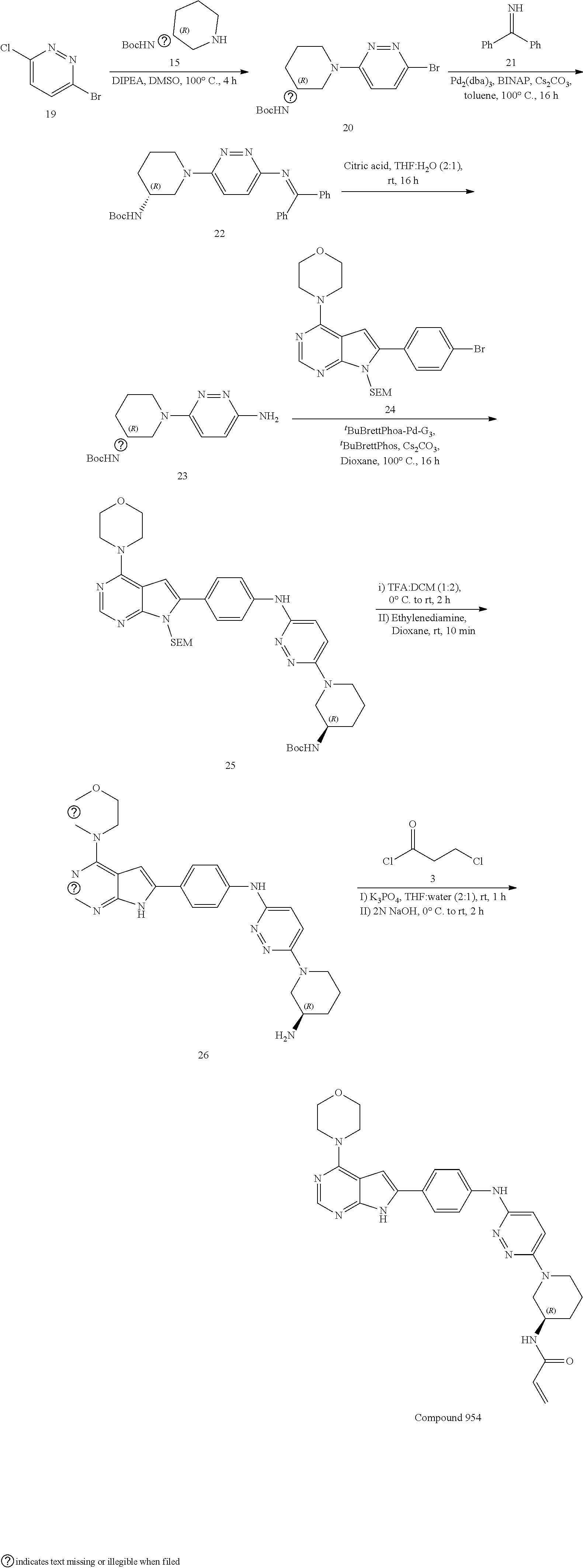

### Synthesis of tert-butyl (R)-(1-(6-bromopyridazin-3-yl)piperidin-3-yl)carbamate (20)

To a stirred solution of 3-bromo-6-chloropyridazine 19 (1.5 g, 9.31 mmol) in DMSO (30 mL) was added DIPEA (5 mL, 27.3 mmol) and tert-butyl (R)-piperidin-3-ylcarbamate 16 (2.8 g, 14 mmol) sequentially at RT under N₂ atmosphere and stirred at 100° C. for 4 h. After completion of reaction (monitored by TLC), the reaction mixture was cooled to RT, poured into ice cold water (100 mL) and extracted with EtOAc (3×80 mL). The combined organic layer was washed with ice cold brine (2×80 mL), dried over Na₂SO₄, filtered and evaporated under reduced pressure to get tittle compound as an off white solid 20 (3 g; crude), which was used in next step directly without further purification.

LCMS=[M+H]⁺: 357.11; Purity=75%

### Synthesis of tert-butyl (R)-(1-(6-((diphenylmethylene)amino)pyridazin-3-yl)piperidin-3-yl)carbamate (22)

To a stirred solution of tert-butyl (R)-(1-(6-bromopyridazin-3-yl)piperidin-3-yl)carbamate 20 (1.5 g, 4.2 mmol) and diphenylmethanimine 21 (1.1 mL, 6.3 mmol) in toluene (20 mL) was added Cs₂CO₃ (4.1 g, 12.6 mmol) at RT. The reaction mixture was degassed with N₂ gas for 10 min, followed by BINAP (523 mg, 0.842 mmol) and Pd₂(dba)₃ (340 g, 0.420 mmol) were added and stirred at 100° C. for 16 h. After completion of reaction (monitored by TLC and LCMS), the reaction mixture was filtered through celite pad and washed with EtOAc (200 mL). The filtrate was washed with water (2×80 mL). The organic layer was dried over Na₂SO₄, filtered and evaporated under reduced pressure. The crude was purified by flash column chromatography (silica gel 100-200 M, 40 g SNAP) using eluents 20-30% EtOAc in heptane to get the title compound as a light yellow sticky solid 22 (940 mg, 49%).

LCMS=[M+H]⁺: 458.30; Purity=90%.

### Synthesis of tert-butyl (R)-(1-(6-aminopyridazin-3-yl)piperidin-3-yl)carbamate (23)

To an ice cold stirred solution of tert-butyl (R)-(1-(6-((diphenylmethylene)amino)pyridazin-3-yl)piperidin-3-yl)carbamate 22 (940 mg, 2.05 mmol) in THF (15 mL) was added a solution of citric acid (9 g, 47.2 mmol) in H₂O (7.5 mL) dropwise at 0° C. under N₂ atmosphere and stirred at RT for 16 h. After completion of reaction (monitored by TLC), the reaction mixture was concentrated under reduced pressure, quenched with sat. NaHCO₃ and extracted with 25% IPA in CHCl₃ (3×80 mL). The combined organic layer over dried over Na2SO₄, filtered and concentrated under reduced pressure. The crude was triturated with Et20, decanted and dried under reduced pressure to get the tittle compound as a yellow semi solid 23 (350 mg; crude) which was used in next step without purification.

LCMS=[M+H]⁺: 294.13; Purity=75%.

### Synthesis of tert-butyl (R)-(1-(6-((4-(4-morpholino-7-((2-(trimethylsilyl)ethoxy)methyl)-7H-pyrrolo[2,3-d]pyrimidin-6-yl)phenyl)amino)pyridazin-3-yl)piperidin-3-yl)carbamate (25)

To a stirred solution of tert-butyl (R)-(1-(6-aminopyridazin-3-yl)piperidin-3-yl)carbamate 23 (300 mg, 1.02 mmol) and 4-(6-(4-bromophenyl)-7-((2-(trimethylsilyl)ethoxy)methyl)-7H-pyrrolo[2,3-d]pyrimidin-4-yl) morpholine 24 (500 mg, 1.02 mmol) in 1,4-dioxane (10 mL) was added Cs₂CO₃ (665 mg, 2.04 mmol) at RT. The reaction mixture was degassed with N₂ gas for 10 min, followed by ^{t}BuBrettPhos (110 mg, 0.205 mmol) and ^{t}BuBrettPhos-Pd-G3 (93 mg, 0.102 mmol) were added sequentially and stirred at 100° C. for 16 h. After completion of reaction (monitored by TLC and LCMS), the reaction mixture was filtered through celite pad and washed with EtOAc (100 mL). The filtrate was washed with water (2×50 mL). The organic layer was separated, dried over Na₂SO₄, filtered and evaporated under reduced pressure to get the title compound as a brown sticky solid 25 (700 mg; crude) which was used in next step without further purification.

LCMS=[M+H]⁺: 702.47; Purity=65%.

### Synthesis of (R)-6-(3-aminopiperidin-1-yl)-N-(4-(4-morpholino-7H-pyrrolo[2,3-d]pyrimidin-6-yl)phenyl)pyridazin-3-amine (26)

To a stirred solution of tert-butyl (R)-(1-(6-((4-(4-morpholino-7-((2-(trimethylsilyl)ethoxy)methyl)-7H-pyrrolo[2,3-d]pyrimidin-6-yl)phenyl)amino)pyridazin-3-yl)piperidin-3-yl)carbamate 25 (700 mg, 0.997 mmol) in DCM (8 mL) was added TFA (4 mL) dropwise under N₂ atmosphere at 0° C. The reaction mixture was stirred at RT for 2 h. After completion of reaction (monitored by TLC and LCMS), the reaction mixture was concentrated under reduced pressure, triturated with Et₂O and dried under reduced pressure. The crude was dissolved in 1,4-dioxane (10 mL) and ethylenediamine (100 μL, 1.5 mmol) was added to the reaction mixture and stirred at RT for 10 min. After completion of reaction (monitored by LCMS), the reaction was poured into ice cold water (50 mL) and extracted with 25% IPA in CHCl₃ (3×70 mL). The combined organic layer was dried over Na₂SO₄, filtered and concentrated under reduced pressure to get the title compound as a brown solid 26 (500 mg; crude), which was used in next step directly without further purification.

LCMS=[M+H]⁺: 472.21; Purity=55%.

### Synthesis of (R)—N-(1-(6-((4-(4-morpholino-7H-pyrrolo[2,3-d]pyrimidin-6-yl)phenyl)amino)pyridazin-3-yl)piperidin-3-yl)acrylamide (Compound 954)

To an ice cold stirred solution of (R)-6-(3-aminopiperidin-1-yl)-N-(4-(4-morpholino-7H-pyrrolo[2,3-d]pyrimidin-6-yl)phenyl)pyridazin-3-amine 26 (500 mg, 1.06 mmol) in THF (6 mL) was added K₃PO₄ (450 mg, 2.12 mmol) dissolved in H₂O (3 mL) under N₂ atmosphere and stirred for 5 min. Then a solution of 3-3-chloropropionyl chloride 3 (162 mg, 1.27 mmol) in THF (1 mL) was added dropwise and stirred at RT for 1 h. After completion of starting material (acylation reaction, monitored by TLC), 2N aq. NaOH (6.5 mL, 12.7 mmol) was added to the reaction mixture at 0° C. and stirred at RT for 2 h. After completion of reaction (complete elimination, monitored by TLC and LCMS), the reaction mixture was diluted with water (30 mL) and extracted with 2-MeTHF (3×80 mL). The combined organic layer was dried over Na₂SO₄, filtered and concentrated under reduced pressure. The crude was purified by RP-HPLC to get the title Compound 954 (95 mg, 17%).

¹H NMR (400 MHz, DMSO-d₆): δ 12.42 (s, 1H), 9.77 (s, 1H), 8.23-8.22 (m, 2H), 7.92-7.85 (m, 3H), 7.69 (d, J=8.4 Hz, 2H), 7.50 (d, J=10.0 Hz, 1H), 7.17 (s, 1H), 6.27-6.20 (m, 1H), 6.12 (dd, J=16.8 Hz, 2 Hz, 1H), 5.63 (dd, J=9.6 Hz, 2.4 Hz, 1H), 4.00-3.97 (m, 1H), 3.91-3.85 (m, 6H), 3.78-3.70 (m, 4H), 3.48-3.40 (m, 1H), 3.31-3.27 (m, 1H), 1.93-1.85 (m, 2H), 1.68-1.57 (m, 2H).

LCMS=[M+H]⁺: 526.19; Purity=97.10%.

### Example 101

### Compound 955

### Preparation of 1-(4-(5-((4-(4-morpholino-1H-imidazo[4,5-c]pyridin-2-yl)phenyl)amino)pyrimidin-2-yl)piperazin-1-yl)prop-2-en-1-one (Compound 955)

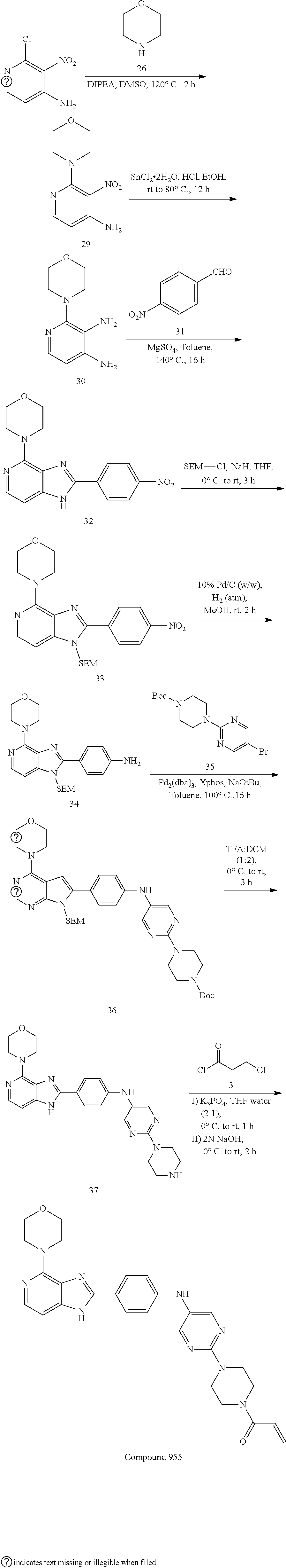

### Synthesis of 2-morpholino-3-nitropyridin-4-amine (29)

To a stirred solution of 2-chloro-3-nitropyridin-4-amine 27 (3 g, 17.3 mmol) and morpholine 28 (2.3 mL, 25.9 mmol) in DMSO (30 mL) was added DIPEA (9.0 mL, 51.9 mmol) at RT under N₂ atmosphere. The reaction mixture was stirred at 120° C. for 2 h. After completion of reaction (monitored by TLC), the reaction mixture was poured into ice water (100 mL) and extracted with EtOAc (3×100 mL). The combined organic layer was washed with ice cold brine solution (2×50 mL), dried over Na₂SO₄, filtered and evaporated under reduced pressure to get the title compound as a yellow solid 29 (2.6 g; crude) which was used in next step directly without further purification.

LCMS=[M+H]⁺: 225.06; Purity=82%.

### Synthesis of 2-morpholinopyridine-3,4-diamine (30)

To a stirred solution of SnCl₂·2H₂O (10.1 g, 44.6 mmol) in Conc. HCl (2.3 mL, 22.3 mmol) was added a solution of 2-morpholino-3-nitro-4-pyridinamine 29 (2.5 g, 11.1 mmol) in EtOH (20 mL) at RT under N₂ atmosphere and stirred at 80° C. for 12 h. After completion of reaction (monitored by TLC), the reaction mixture was concentrated under reduce pressure. The residue was diluted with ice cold water (50 mL) and basified with sat. NaHCO₃ solution to pH~8-9 and extracted with 25% IPA in CHCl₃ (3×80 mL). The combined organic layer was washed with brine (2×50 mL), dried over Na₂SO₄, filtered and evaporated under reduced pressure to get the title compound as a red solid 30 (1.1 g; crude), which was used in next step directly without further purification.

LCMS=[M+H]⁺: 195.14; Purity=80%.

### Synthesis of 4-(2-(4-nitrophenyl)-1H-imidazo[4,5-c]pyridin-4-yl)morpholine (32)

To a stirred solution of 2-morpholino-3,4-pyridinediamine 30 (1 g, 5.15 mmol) in toluene (15 mL) was added MgSO₄ (620 mg, 5.15 mmol) and p-nitrobenzaldehyde 31 (934 mg, 6.18 mmol) at RT under N₂ atmosphere. The reaction mixture was stirred at 140° C. for 16 h. After completion of reaction (monitored by TLC and LCMS), the reaction mixture was filtered and washed with 25% IPA in CHCl₃ (100 mL). The filtrate was concentrated under reduced pressure to get the title compound as a red solid 32 (850 mg; crude), which was used in next step directly without further purification.

LCMS=[M+H]⁺: 326.14; Purity=55%.

### Synthesis of 4-(2-(4-nitrophenyl)-1-((2-(trimethylsilyl)ethoxy)methyl)-1H-imidazo[4,5-c]pyridin-4-yl)morpholine (33)

To a stirred solution of 4-morpholino-2-(p-nitrophenyl)-1H-1,3,5-triazaindene 32 (850 mg, 2.61 mmol) in THF (10 mL) was added NaH (157 mg, 3.92 mmol; 60% dispersion in mineral oil) at 0° C. under N₂ atmosphere and stirred for 30 min. Then SEM-C1 (695 μL, 3.92 mmol) was added at 0° C. and stirred at RT for 3 h. After completion of reaction (monitored by TLC), the reaction mixture was quenched with ice cold water (100 mL) and extracted with EtOAc (3×80 mL). The combined organic layer was washed brine (2×50 mL), dried over Na₂SO₄, filtered and concentrated under reduced pressure. The crude was purified by flash chromatography (silica gel 100-200 M, 12 g SNAP) using eluents 30-40% EtOAc in heptane to get the title compound as a light yellow solid 33 (500 mg, 42%).

LCMS=[M+H]⁺: 456.26; Purity=84%.

### Synthesis of 4-(4-morpholino-1-((2-(trimethylsilyl)ethoxy)methyl)-1H-imidazo[4,5-c]pyridin-2-yl)aniline (34)

To a stirred solution of 4-(2-(4-nitrophenyl)-1-((2-(trimethylsilyl)ethoxy)methyl)-1H-imidazo[4,5-c]pyridin-4-yl)morpholine 33 (500 mg, 1.1 mmol) in MeOH (10 mL) was added 10% Pd—C(500 mg) at RT under H₂ atmosphere and stirred at RT for 2 h. After completion of reaction (monitored by TLC), the reaction mixture was filtered through celite bed and washed with MeOH (50 mL). The filtrate was concentrated under reduce pressure to get the title compound as a light yellow solid 34 (350 mg, 71%). which was used in next step directly without further purification.

LCMS=[M+H]⁺: 426.28; Purity=98%.

### Synthesis of tert-butyl 4-(5-((4-(4-morpholino-1-((2-(trimethylsilyl)ethoxy)methyl)-1H-imidazo[4,5-c]pyridin-2-yl)phenyl)amino)pyrimidin-2-yl)piperazine-1-carboxylate (36)

To a stirred solution of 4-(4-morpholino-1-((2-(trimethylsilyl)ethoxy)methyl)-1H-imidazo[4,5-c]pyridin-2-yl)aniline 34 (330 mg, 0.775 mmol) and tert-butyl 4-(5-bromopyrimidin-2-yl)piperazine-1-carboxylate 35 (319 mg, 0.930 mmol) in toluene (10 mL) was added NaO^{t}Bu (112 mg, 1.16 mmol) at RT. The reaction mixture was purged with N₂ gas for 10 min, followed by Pd₂(dba)₃ (71 mg, 0.775 mmol) and XPhos (37 mg, 0.775 mmol) was added. The reaction mixture was stirred at 100° C. for 16 h. After completion of reaction (monitored by TLC and LCMS), the reaction mixture was diluted with water (20 mL) and extracted with EtOAc (3×30 mL). The combined organic layer was dried over Na₂SO₄, filtered and evaporated under reduced pressure. The crude was purified by flash chromatography (silica gel 100-200 M, 12 g SNAP) using eluents 25-30% EtOAc in heptane to get the title compound as a brown solid 36 (250 mg, 47%).

LCMS=[M+H]⁺: 688.46; Purity=96%.

### Synthesis of N-(4-(4-morpholino-1H-imidazo[4,5-c]pyridin-2-yl)phenyl)-2-(piperazin-1-yl)pyrimidin-5-amine (37)

To a stirred solution of tert-butyl 4-(5-((4-(4-morpholino-1-((2-(trimethylsilyl)ethoxy)methyl)-1H-imidazo[4,5-c]pyridin-2-yl)phenyl)amino)pyrimidin-2-yl)piperazine-1-carboxylate 36 (250 mg, 0.363 mmol) in DCM (5 mL) was added TFA (2.5 mL) dropwise at 0° C. under N₂ atmosphere. The reaction mixture was stirred at RT for 3 h. After completion of reaction (monitored by TLC and LCMS), the reaction mixture was concentrated under reduced pressure, triturated with Et₂O and dried under reduced pressure to get the title compound as a brown solid 37 (160 mg; crude), which was used in next step directly without further purification.

LCMS=[M+H]⁺: 458.27; Purity=50%.

### Synthesis of 1-(4-(5-((4-(4-morpholino-1H-imidazo[4,5-c]pyridin-2-yl)phenyl)amino)pyrimidin-2-yl)piperazin-1-yl)prop-2-en-1-one (Compound 955)

To a stirred solution of N-(4-(4-morpholino-1H-imidazo[4,5-c]pyridin-2-yl)phenyl)-2-(piperazin-1-yl)pyrimidin-5-amine 37 (160 mg, 0.349 mmol) in THF (4 mL) was added K₃PO₄ (148 mg, 0.697 mmol) dissolved in water (2 mL) at 0° C. under N₂ atmosphere and stirred for 5 min. Then a solution of 3-chloropropionyl chloride 3 (40 μL, 0.315 mmol) in THF (1 mL) was added drop wise to the reaction mixture at 0° C. and stirred at RT for 1 h. After completion of starting material (acylation reaction, monitored by TLC), 2N aq. NaOH (2 mL, 4.18 mmol) was added to the reaction mixture at 0° C. and stirred at RT for 2 h. After completion of reaction (complete elimination, monitored by TLC and LCMS), the reaction mixture was diluted with water (10 mL) and extracted with 2-MeTHF (3×40 mL). The combined organic layer was dried over Na2SO₄, filtered and concentrated under reduced pressure. The crude was purified by RP-HPLC [5-95% 0.1% TFA in Water/ACN; Column: X Select CSH C18 (19*250) 10μ] to get the title Compound 955 (7 mg, 14%).

¹H NMR (400 MHz, DMSO-d₆): δ 13.74 (s, 1H), 8.37 (s, 2H), 8.32 (s, 1H), 7.93 (d, J=8.4 Hz, 2H), 7.69 (d, J=6.8 Hz, 1H), 7.21-7.09 (m, 1H), 6.96-6.91 (m, 2H), 6.89-6.82 (m, 1H), 6.15 (d, J=16.8 Hz, 2.4 Hz, 1H), 5.72 (d, J=10.4 Hz, 2.0 Hz, 1H), 4.23-4.19 (m, 4H), 3.87-3.84 (m, 4H), 3.78-3.74 (m, 4H), 3.68-3.65 (m, 4H).

LCMS=[M+H]⁺: 512.17; Purity=99.34%.

### Example 102

### Compound 956

### Preparation of (E)-4-(dimethylamino)-1-(4-(5-((4-(4-morpholino-1H-pyrrolo[3,2-c]pyridin-2-yl)phenyl)amino)pyrimidin-2-yl)piperazin-1-yl)but-2-en-1-one (Compound 956)

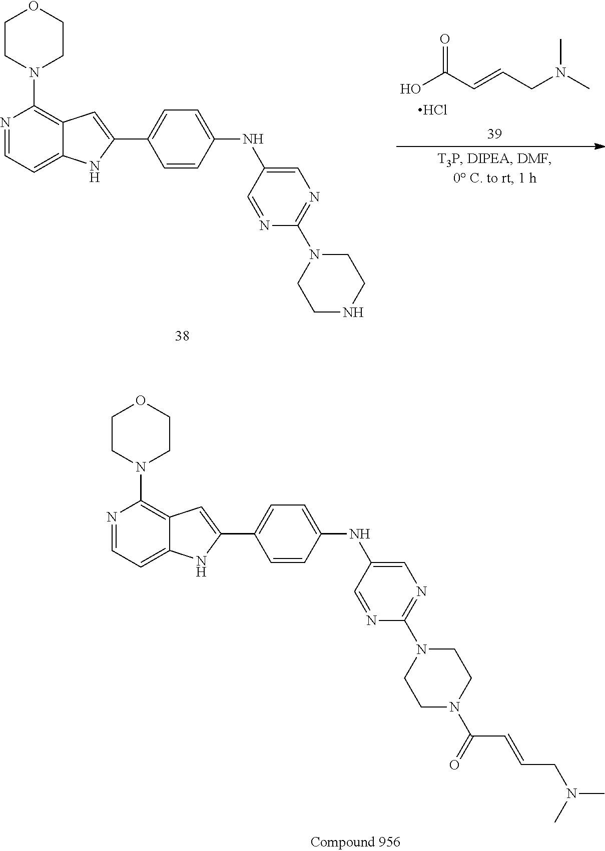

To a stirred solution of N-(4-(4-morpholino-1H-pyrrolo[3,2-c]pyridin-2-yl)phenyl)-2-(piperazin-1-yl)pyrimidin-5-amine 38 (300 mg, 0.657 mmol) and (E)-4-(dimethylamino)but-2-enoic acid·HCl 39 (163 mg, 0.986 mmol) in DMF (5 mL) were added DIPEA (340 μL, 1.97 mmol) and T3P (540 μL, 0.854 mmol) sequentially at 0° C. under N₂ atmosphere and stirred at RT for 1 h. After completion of reaction (monitored by TLC), the reaction mixture was poured into ice cold water (50 mL) and extracted with 25% IPA in CHCl₃ (3×50 mL). The combined organic layer was washed with ice cold brine solution (2×50 mL), dried over Na₂SO₄, filtered and concentrated under reduced pressure. The crude was purified by RP-HPLC [5-95% 0.1% TFA in Water/ACN; Column: X Select Fluoro Phenyl (19*250) 5μ] to get the title Compound 956 (30 mg, 8%).

¹H NMR (400 MHz, DMSO-d₆): δ 12.66 (s, 1H), 9.84 (s, 1H), 8.36 (s, 1H), 8.14 (s, 1H), 7.74 (d, J=8.4 Hz, 2H), 7.62 (d, J=6.8 Hz, 1H), 7.31 (brs, 1H), 7.14 (d, J=6.8 Hz, 1H), 6.99-6.97 (m, 1H), 6.90 (d, J=8.8 Hz, 2H), 6.67-6.60 (m, 1H), 3.89 (d, J=7.2 Hz, 2H), 3.83 (s, 8H), 3.77-3.74 (m, 4H), 3.69-3.64 (m, 4H), 2.79 (s, 6H).

LCMS=[M+H]⁺: 568.32; Purity=95.27%.

### Example 103

### Compound 957

### Preparation of (R)-N-methyl-N-(1-((5-((4-(4-morpholino-7H-pyrrolo[2,3-d]pyrimidin-6-yl)phenyl)amino)pyrimidin-2-yl)methyl)piperidin-3-yl)acrylamide (Compound 957)

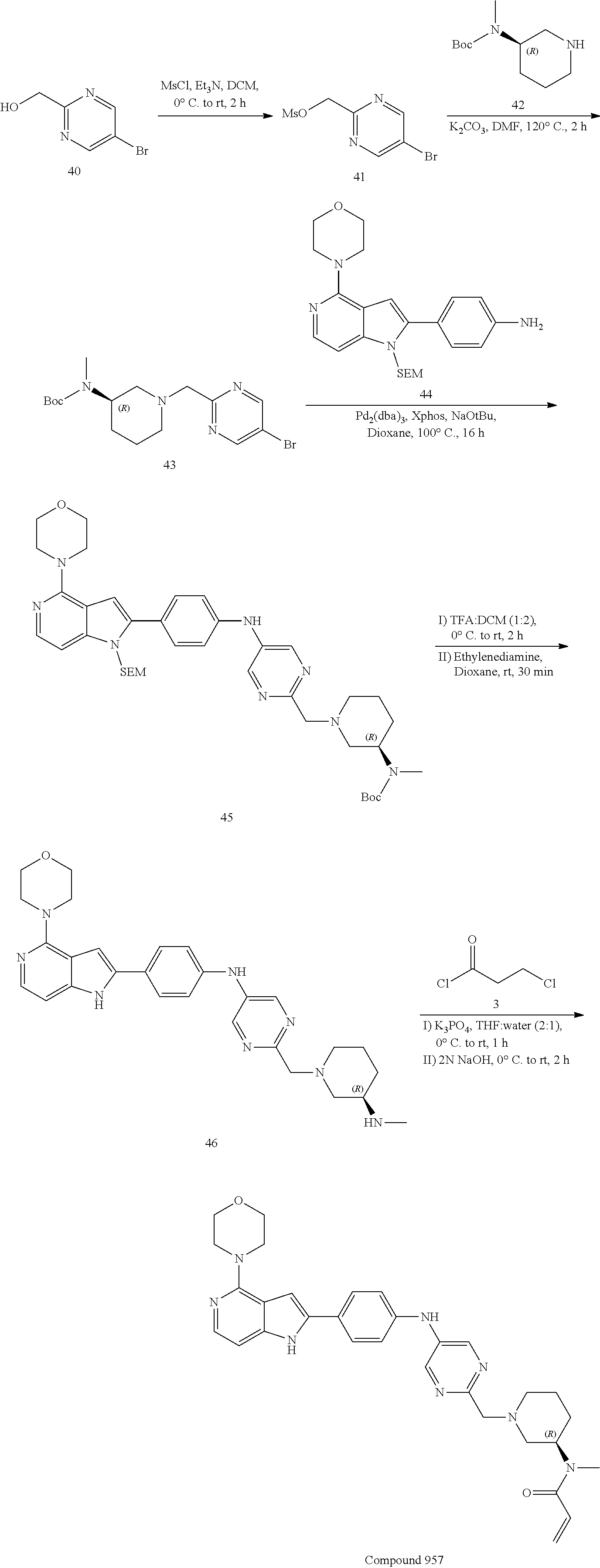

### Synthesis of (5-bromopyrimidin-2-yl)methyl methanesulfonate (41)

To a stirred solution of (5-bromopyrimidin-2-yl)methanol 40 (3 g, 15.9 mmol) in DCM (50 mL) was added Et₃N (6.6 mL, 47.6 mmol) and MsCl (1.85 mL, 23.85 mmol) at 0° C. under N₂ atmosphere. The reaction mixture was stirred at RT for 2 h. After completion of reaction (monitored by TLC), the reaction mixture was quenched with water (50 mL) and extracted with DCM (3×80 mL). The combined organic layer was dried over Na₂SO₄, filtered and concentrated under reduced pressure to get the title compound as brown sticky oil 41 (3 g; crude), which was directly used in the next step without any purification.

LCMS=[M+H]⁺: 268.92; Purity=76%.

### Synthesis of tert-butyl (R)-(1-((5-bromopyrimidin-2-yl)methyl)piperidin-3-yl)(methyl)carbamate (43)

To a stirred solution of (5-bromopyrimidin-2-yl)methyl methanesulfonate 41 (1.3 g, 4.87 mmol) in DMF (20 mL) was added K₂CO₃ (3.4 g, 24.3 mmol) and tert-butyl (R)-methyl (piperidin-3-yl)carbamate 42 (2 g, 9.73 mmol) at 0° C. under N₂ atmosphere. The reaction mixture was stirred at 120° C. for 2 h. After completion of reaction (monitored by TLC), the reaction mixture was poured into ice cold water (100 mL) and extracted with EtOAc (3×80 mL). The combined organic layer was dried over Na₂SO₄, filtered and concentrated under reduced pressure. The crude was purified by flash column chromatography (silica gel 100-200 M, 24 g SNAP) using eluents 20-30% EtOAc in heptane to get the title compound as a brown solid 43 (1 g; 53%).

LCMS=[M+H]⁺: 385.15; Purity=85%.

### Synthesis of tert-butyl (R)-methyl (1-((5-((4-(4-morpholino-7-((2-(trimethylsilyl)ethoxy)methyl)-7H-pyrrolo[2,3-d]pyrimidin-6-yl)phenyl)amino)pyrimidin-2-yl)methyl)piperidin-3-yl)carbamate (45)

To a stirred solution of tert-butyl (R)-(1-((5-bromopyrimidin-2-yl)methyl)piperidin-3-yl)(methyl)carbamate 43 (1 g, 2.6 mmol) and 4-(4-morpholino-7-((2-(trimethylsilyl)ethoxy)methyl)-7H-pyrrolo[2,3-d]pyrimidin-6-yl)aniline 44 (1.1 g, 2.6 mmol) in 1,4-dioxane (20 mL) was added NaO^{t}Bu (374 mg, 3.89 mmol) at RT. The reaction mixture was degassed with N₂ gas for 10 min, followed by Pd₂(dba)₃ (210 mg, 0.260 mmol) and XPhos (124 mg, 0.260 mmol) were added and stirred at 100° C. for 16 h. After completion of reaction (monitored by TLC and LCMS), the reaction mixture was diluted with water (100 mL) and extracted with EtOAc (3×80 mL). The combined organic layer was dried over Na₂SO₄, filtered and concentrated under reduced pressure. The crude was purified by flash column chromatography (silica gel 100-200 M, 24 g SNAP) using eluents 25-35% EtOAc in heptane to get the title compound as a brown solid 45 (1.2 g, 63%).

LCMS=[M+H]⁺: 730.21; Purity=67%.

### Synthesis of (R)-2-((3-(methylamino)piperidin-1-yl)methyl)-N-(4-(4-morpholino-7H-pyrrolo[2,3-d]pyrimidin-6-yl)phenyl)pyrimidin-5-amine (46)

To a stirred solution of tert-butyl (R)-methyl (1-((5-((4-(4-morpholino-7-((2-(trimethylsilyl)ethoxy)methyl)-7H-pyrrolo[2,3-d]pyrimidin-6-yl)phenyl)amino)pyrimidin-2-yl)methyl)piperidin-3-yl)carbamate 45 (1.2 g, 1.64 mmol) in DCM (20 mL) was added TFA (10 mL) dropwise at 0° C. under N₂ atmosphere and stirred at RT for 2 h. After completion of reaction (monitored by TLC), the reaction mixture was concentrated under reduced pressure, triturated with Et₂O and dried under reduced pressure. The crude was dissolved in 1,4-dioxane (10 mL) and ethylenediamine (165 μL, 2.47 mmol) was added to the reaction mixture and stirred at RT for 30 min. After completion of reaction (monitored by LCMS), the reaction was poured into ice cold water (100 mL) and extracted with 25% IPA in CHCl₃ (3×100 mL). The combined organic layer was dried over Na₂SO₄, filtered and concentrated under reduced pressure to get the title compound as a brown solid 46 (750 mg; crude), which was used in next step directly without further purification.

LCMS=[M+H]⁺: 500.27; Purity=63%.

### Synthesis of (R)-N-methyl-N-(1-((5-((4-(4-morpholino-7H-pyrrolo[2,3-d]pyrimidin-6-yl)phenyl)amino)pyrimidin-2-yl)methyl)piperidin-3-yl)acrylamide (Compound 957)

To a stirred solution of (R)-2-((3-(methylamino)piperidin-1-yl)methyl)-N-(4-(4-morpholino-7H-pyrrolo[2,3-d]pyrimidin-6-yl)phenyl)pyrimidin-5-amine 46 (750 mg, 1.50 mmol) in THF (10 mL) was added K₃PO₄ (636 mg, 3 mmol) dissolved in water (5 mL) at 0° C. under N₂ atmosphere and stirred for 5 min. Then a solution of 3-chloropropionyl chloride 3 (172 μL, 1.8 mmol) in THF (1 mL) was added drop wise to the reaction mixture at 0° C. and stirred at RT for 1 h. After completion of starting material (acylation reaction, monitored by TLC), 2N aq. NaOH (9 mL, 18 mmol) was added to the reaction mixture at 0° C. and stirred at RT for 2 h. After completion of reaction (complete elimination, monitored by TLC and LCMS), the reaction mixture was diluted with water (80 mL) and extracted with 2-MeTHF (3×80 mL). The combined organic layer was dried over Na2SO₄, filtered and concentrated under reduced pressure. The crude was purified by flash column chromatography (silica gel 100-200 M, 12 g SNAP) using eluents 5-7% MeOH in DCM to get the title Compound 957 (180 mg, 22%).

¹H NMR (400 MHz, DMSO-d₆): δ 12.10 (s, 1H), 8.64 (s, 1H), 8.57 (s, 2H), 8.16 (s, 1H), 7.83 (d, J=8.8 Hz, 2H), 7.17 (d, J=8.0 Hz, 2H), 7.04 (d, J=2.0 Hz, 1H), 6.74-6.67 (m, 1H), 6.10-6.00 (m, 1H), 5.64 (t, J=9.2 Hz, 1H), 4.43-4.36 (m, 1H), 3.88-3.86 (m, 4H), 3.75-3.73 (m, 4H), 3.64-3.61 (m, 2H), 2.83-2.77 (m, 5H), 2.32-2.11 (m, 1H), 1.99 (t, J=10.4 Hz, 1H), 1.67-1.47 (m, 4H).

LCMS=[M+H]⁺: 554.24; Purity=96.78%.

### Example 104

### Compound 958 and Compound 959

### Preparation of 1-((R)-3-(4-((S)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 958) and 1-((S)-3-(4-((S)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 959)

Chiral separation of Compound 939 resulted in both enantiomers 1-((R)-3-(4-((S)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 958) and 1-((S)-3-(4-((S)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 959)

Note: Chirality arbitrarily assigned

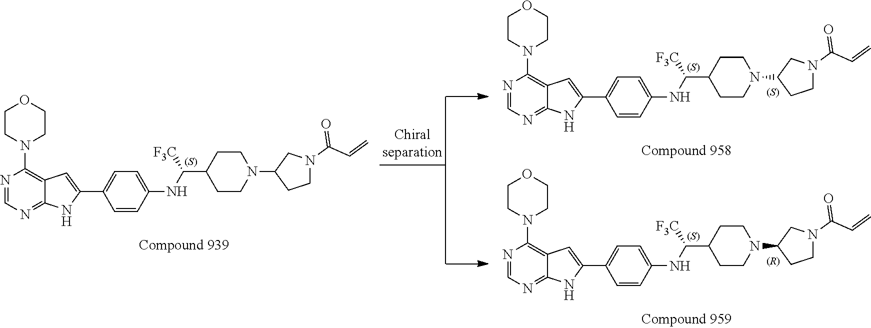

### SFC Purification Conditions:

Column: CHIRALCEL OJ-H (30×250 mm; 5u)

Mobile Phase: i) CO₂
ii) 0.2% TEA in ACN: MeOH (1:1)

Flow rate: Isocratic 100 mL/Min, wavelength: 263 nm.

Analytical data of Compound 958:

¹H NMR (400 MHz, DMSO-d₆): δ 11.95 (s, 1H), 8.13 (s, 1H), 7.64 (d, J=8.4 Hz, 2H), 6.87-6.84 (m, 3H), 6.60-6.51 (m, 1H), 6.13-6.06 (m, 2H), 5.66-5.61 (m, 1H), 4.30-4.28 (m, 1H), 3.85-3.83 (m, 4H), 3.74-3.66 (m, 5H), 3.55-3.40 (m, 1H), 3.22-3.17 (m, 1H), 2.99-2.96 (m, 1H), 2.83-2.78 (m, 2H), 2.13-2.04 (m, 1H), 2.00-1.92 (m, 2H), 1.79-1.62 (m, 4H), 1.59-1.44 (m, 3H).

LCMS=[M+H]⁺: 584.18; Purity=97.26%.

Analytical data of Compound 959:

¹H NMR (400 MHz, DMSO-d₆): δ 11.95 (s, 1H), 8.13 (s, 1H), 7.64 (d, J=8.4 Hz, 2H), 6.88-6.84 (m, 3H), 6.62-6.51 (m, 1H), 6.13-6.05 (m, 2H), 5.66-5.61 (m, 1H), 4.31-4.26 (m, 1H), 3.85-3.83 (m, 4H), 3.75-3.66 (m, 5H), 3.57-3.40 (m, 1H), 3.23-3.18 (m, 1H), 3.00-2.79 (m, 4H), 2.13-2.07 (m, 1H), 2.05-1.92 (m, 2H), 1.82-1.62 (m, 4H), 1.60-1.46 (m, 3H).

LCMS=[M+H]⁺: 584.20; Purity=96.31%.

### Example 105

### Compound 960

### Preparation of 1-(4-((5-((4-(4-morpholino-7H-pyrrolo[2,3-d]pyrimidin-6-yl)phenyl)amino)pyrimidin-2-yl)methyl)piperazin-1-yl)prop-2-en-1-one (Compound 960)

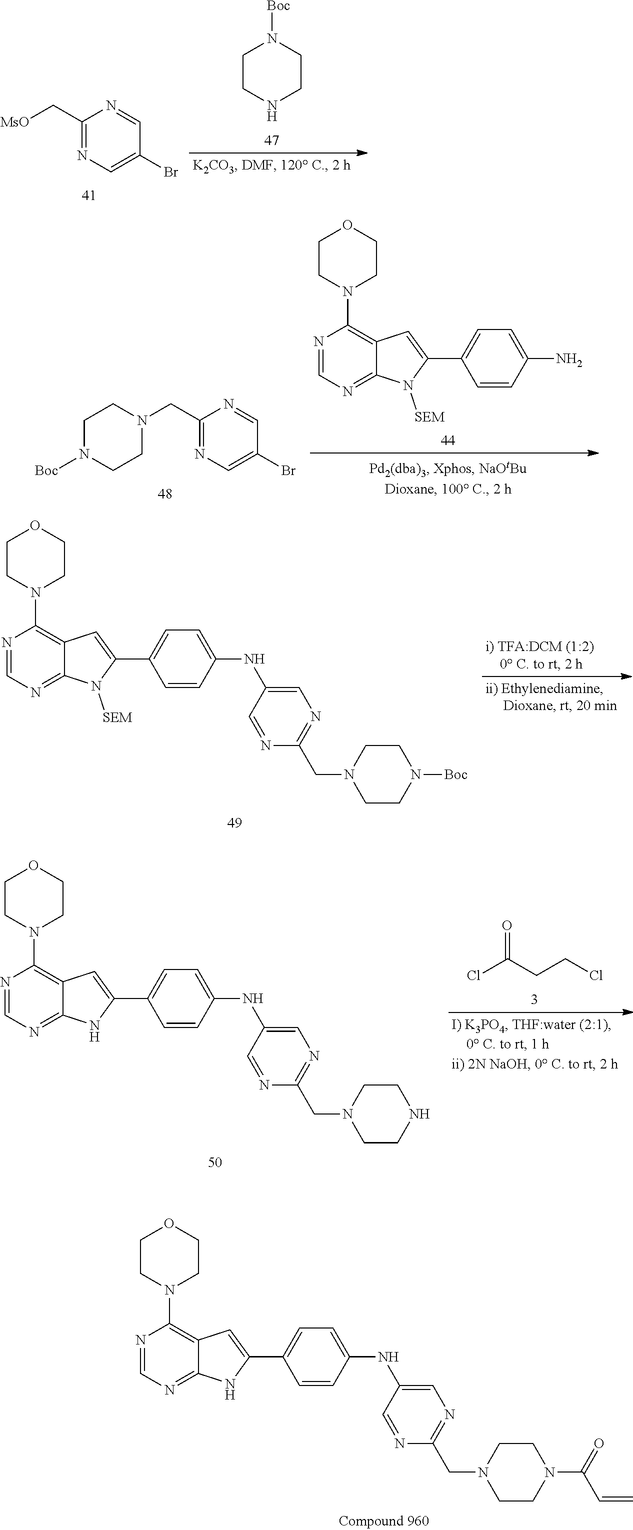

### Synthesis of tert-butyl 4-((5-bromopyrimidin-2-yl)methyl)piperazine-1-carboxylate (48)

To a stirred solution of (5-bromopyrimidin-2-yl)methyl methanesulfonate 41 (1 g, 3.74 mmol) in DMF (10 mL) were added K₂CO₃ (2.59 g, 18.7 mmol) and tert-butyl piperazine-1-carboxylate 47 (1.5 g, 7.49 mmol) sequentially at RT under N₂ atmosphere and stirred at 120° C. for 2 h. After completion of reaction (monitored by TLC), the reaction mixture was cooled to RT, poured into ice cold water (100 mL) and extracted with EtOAc (3×80 mL). The combined organic layer was washed with ice cold brine (2×80 mL), dried over Na₂SO₄, filtered and evaporated under reduced pressure. The crude was purified by flash column chromatography (silica gel 100-200 M, 24 g SNAP) using eluents 20-30% EtOAc in heptane to get the title compound as an orange solid 48 (1.2 g, 85%).

LCMS=[M+H]⁺: 357.12; Purity=98%.

### Synthesis of tert-butyl 4-((5-((4-(4-morpholino-7-((2-(trimethylsilyl)ethoxy)methyl)-7H-pyrrolo[2,3-d]pyrimidin-6-yl)phenyl)amino)pyrimidin-2-yl)methyl)piperazine-1-carboxylate (49)

To a stirred solution of tert-butyl 4-((5-bromopyrimidin-2-yl)methyl)piperazine-1-carboxylate 48 (600 mg, 1.62 mmol) and 4-(4-morpholino-7-((2-(trimethylsilyl)ethoxy)methyl)-7H-pyrrolo[2,3-d]pyrimidin-6-yl)aniline 44 (690 mg, 1.62 mmol) in 1,4-dioxane (10 mL) was added NaO^{t}Bu (0.233 g, 2.42 mmol) at RT. The reaction mixture was degassed with N₂ gas for 10 min, followed by Pd₂(dba)₃ (131 mg, 0.162 mmol) and XPhos (77 mg, 0.162 mmol) were added and stirred at 100° C. for 2 h. After completion of reaction (monitored by TLC and LCMS), the reaction mixture was diluted with water (60 mL) and extracted with EtOAc (3×80 mL). The combined organic layer was dried over Na₂SO₄, filtered and concentrated under reduced pressure. The crude was purified by flash column chromatography (silica gel 100-200 M, 12 g SNAP) using eluents 25-35% EtOAc in heptane to get the title compound as a brown solid 49 (1 g, 86%).

LCMS=[M+H]⁺: 702.43; Purity=65%.

### Synthesis of N-(4-(4-morpholino-7H-pyrrolo[2,3-d]pyrimidin-6-yl)phenyl)-2-(piperazin-1-ylmethyl)pyrimidin-5-amine (50)

To a stirred solution of tert-butyl 4-((5-((4-(4-morpholino-7-((2-(trimethylsilyl)ethoxy)methyl)-7H-pyrrolo[2,3-d]pyrimidin-6-yl)phenyl)amino)pyrimidin-2-yl)methyl)piperazine-1-carboxylate 49 (1 g, 1.42 mmol) in DCM (12 mL) was added TFA (6 mL) dropwise at 0° C. under N₂ atmosphere and stirred at RT for 2 h. After completion of reaction (monitored by TLC), the reaction mixture was concentrated under reduced pressure, triturated with Et₂O and dried under reduced pressure. The crude was dissolved in 1,4-dioxane (10 mL) and ethylenediamine (143 μL, 2.13 mmol) was added to the reaction mixture and stirred at RT for 20 min. After completion of reaction (monitored by LCMS), the reaction was poured into ice cold water (50 mL) and extracted with 25% IPA in CHCl₃ (3×80 mL). The combined organic layer was dried over Na₂SO₄, filtered and concentrated under reduced pressure to get the title compound as a brown solid 50 (600 mg: crude), which was used in next step directly without further purification.

LCMS=[M+H]⁺: 472.21; Purity=68%.

### Synthesis of 1-(4-((5-((4-(4-morpholino-7H-pyrrolo[2,3-d]pyrimidin-6-yl)phenyl)amino)pyrimidin-2-yl)methyl)piperazin-1-yl)prop-2-en-1-one (Compound 960)

To a stirred solution of N-(4-(4-morpholino-7H-pyrrolo[2,3-d]pyrimidin-6-yl)phenyl)-2-(piperazin-1-ylmethyl)pyrimidin-5-amine 50 (600 mg, 1.27 mmol) in THF (8 mL) was added K₃PO₄ (540 mg, 2.54 mmol) dissolved in water (4 mL) at 0° C. under N₂ atmosphere and stirred for 5 min. Then a solution of 3-chloropropionyl chloride 3 (146 μL, 1.53 mmol) in THF (1 mL) was added drop wise to the reaction mixture at 0° C. and stirred at RT for 1 h. After completion of starting material (acylation reaction, monitored by TLC), 2N aq. NaOH (7.6 mL, 15.2 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×50 mL). The combined organic layer was dried over Na2SO₄, filtered and concentrated under reduced pressure. The crude was purified by RP-HPLC [5-95% 5 mM Ammonium Bicarbonate in Water/ACN; Column: X Select Phenyl Hexyl (19*250) 5μ] to get the title Compound 960 (122 mg, 18%).

¹H NMR (400 MHz, DMSO-d₆): & 12.12 (s, 1H), 8.65 (s, 1H), 8.58 (s, 2H), 8.16 (s, 1H), 7.83 (d, J=8.4 Hz, 2H), 7.17 (d, J=8.8 Hz, 2H), 7.04 (d, J=2.0 Hz, 1H), 6.78 (dd, J=16.4 Hz, 10.4 Hz, 1H), 6.09 (dd, J=16.8 Hz, 2 Hz, 1H), 6.09 (dd, J=16.8 Hz, 2 Hz, 1H), 5.66 (dd, J=10.4 Hz, 2.4 Hz, 1H), 3.88-3.86 (m, 4H), 3.75-3.73 (m, 4H), 3.66 (s, 2H), 3.58-3.48 (m, 4H). [4H are merged in solvent peak]

LCMS=[M+H]⁺: 526.25; Purity=99.04%.

### Example 106

### Compound 961

### Preparation of (R)—N-(1-((5-((4-(4-morpholino-7H-pyrrolo[2,3-d]pyrimidin-6-yl)phenyl)amino)pyrimidin-2-yl)methyl)piperidin-3-yl)acrylamide (Compound 961)

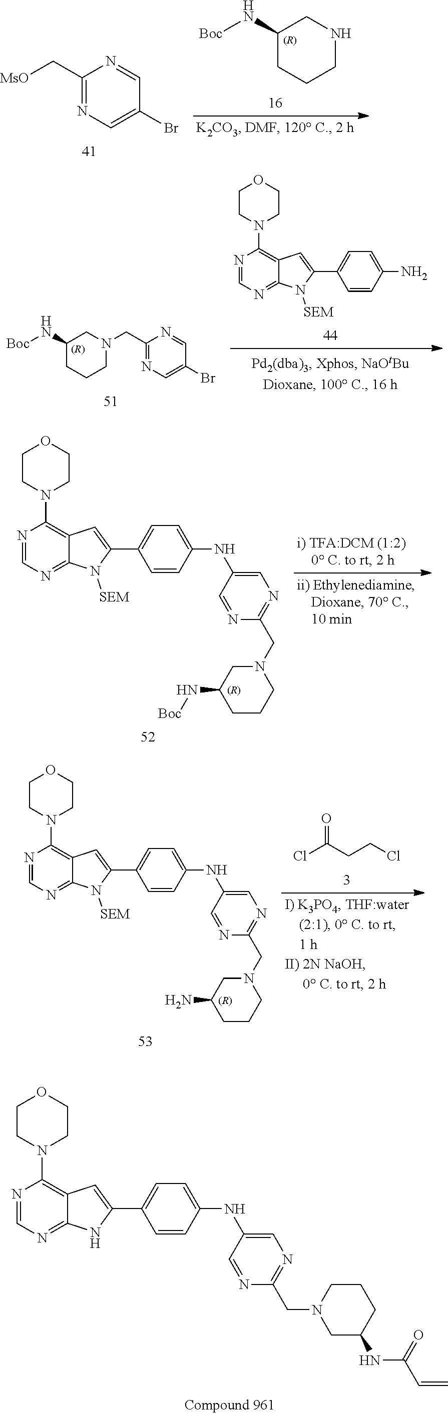

### Synthesis of tert-butyl (R)-(1-((5-bromopyrimidin-2-yl)methyl)piperidin-3-yl)carbamate (51)

To a stirred solution of (5-bromopyrimidin-2-yl)methyl methanesulfonate 41 (1 g, 3.74 mmol) in DMF (10 mL) were added K₂CO₃ (2.6 g, 18.7 mmol) and tert-butyl (R)-piperidin-3-ylcarbamate 16 (1.5 g, 7.49 mmol) sequentially at RT under N₂ atmosphere and stirred at 120° C. for 2 h. After completion of reaction (monitored by TLC), the reaction mixture was cooled to RT, poured into ice cold water (50 mL) and extracted with EtOAc (3×80 mL). The combined organic layer was washed with ice cold brine (2×50 mL), dried over Na₂SO₄, filtered and evaporated under reduced pressure. The crude was purified by flash column chromatography (silica gel 100-200 M, 12 g SNAP) using eluents 20-30% EtOAc in heptane to get the title compound as an orange solid 51 (1.2 g, 85%).

LCMS=[M+H]⁺: 371.07; Purity=99%.

### Synthesis of tert-butyl (R)-(1-((5-((4-(4-morpholino-7-((2-(trimethylsilyl)ethoxy)methyl)-7H-pyrrolo[2,3-d]pyrimidin-6-yl)phenyl)amino)pyrimidin-2-yl)methyl)piperidin-3-yl)carbamate (52)

To a stirred solution of tert-butyl (R)-(1-((5-bromopyrimidin-2-yl)methyl)piperidin-3-yl)carbamate 51 (600 mg, 1.62 mmol) and 4-(4-morpholino-7-((2-(trimethylsilyl)ethoxy)methyl)-7H-pyrrolo[2,3-d]pyrimidin-6-yl)aniline 44 (690 mg, 1.62 mmol) in 1,4-dioxane (10 mL) was added NaO^{t}Bu (233 mg, 2.42 mmol) at RT. The reaction mixture was degassed with N₂ gas for 10 min, followed by Pd₂(dba)₃ (131 mg, 0.162 mmol) and XPhos (77 mg, 0.162 mmol) were added and stirred at 100° C. for 2 h. After completion of reaction (monitored by TLC and LCMS), the reaction mixture was diluted with water (50 mL) and extracted with EtOAc (3×80 mL). The combined organic layer was dried over Na₂SO₄, filtered and concentrated under reduced pressure. The crude was purified by flash column chromatography (silica gel 100-200 M, 12 g SNAP) using eluents 25-35% EtOAc in heptane to get the title compound as a brown solid 52 (1 g, 86%).

LCMS=[M+H]⁺: 716.48; Purity=57%.

### Synthesis of (R)-2-((3-aminopiperidin-1-yl)methyl)-N-(4-(4-morpholino-7H-pyrrolo[2,3-d]pyrimidin-6-yl)phenyl)pyrimidin-5-amine (53)

To a stirred solution of tert-butyl (R)-(1-((5-((4-(4-morpholino-7-((2-(trimethylsilyl)ethoxy)methyl)-7H-pyrrolo[2,3-d]pyrimidin-6-yl)phenyl)amino)pyrimidin-2-yl)methyl)piperidin-3-yl)carbamate 52 (1 g, 1.39 mmol) in DCM (16 mL) was added TFA (8 mL) dropwise at 0° C. and stirred at RT for 2 h. After completion of reaction (monitored by TLC), the reaction mixture was concentrated under reduced pressure, triturated with Et₂O and dried under reduced pressure. The obtained crude was dissolved in 1,4-dioxane (10 mL) and ethylenediamine (140 μL, 2.10 mmol) stirred at 70° C. for 10 min. After completion of reaction (monitored by LCMS), the reaction was poured into ice cold water (100 mL) and extracted with 25% IPA in CHCl₃ (3×80 mL). The combined organic layer was dried over Na₂SO₄, filtered and concentrated under reduced pressure to get the title compound as a brown solid 53 (600 mg; crude), which was used in next step directly without further purification.

LCMS=[M+H]⁺: 486.40; Purity=65%.

### Synthesis of (R)—N-(1-((5-((4-(4-morpholino-7H-pyrrolo[2,3-d]pyrimidin-6-yl)phenyl)amino)pyrimidin-2-yl)methyl)piperidin-3-yl)acrylamide (Compound 961)

To an ice cold stirred solution of (R)-2-((3-aminopiperidin-1-yl)methyl)-N-(4-(4-morpholino-7H-pyrrolo[2,3-d]pyrimidin-6-yl)phenyl)pyrimidin-5-amine 53 (600 mg, 1.24 mmol) in THF (10 mL) was added K₃PO₄ (525 mg, 2.57 mmol) dissolved in water (5 mL) under N₂ atmosphere and stirred for 5 min. Then a solution of 3-chloropropionyl chloride 3 (142 μL, 1.58 mmol) in THF (1 mL) was added drop wise to the reaction mixture at 0° C. and stirred at RT for 1 h. After completion of starting material (acylation reaction, monitored by TLC), 2N aq. NaOH (7.5 mL, 14.88 mmol) was added to the reaction mixture at 0° C. and stirred at RT for 2 h. After completion of reaction (complete elimination, monitored by TLC and LCMS), the reaction mixture was diluted with water (60 mL) and extracted with 2-MeTHF (3×90 mL). The combined organic layer was dried over Na₂SO₄, filtered and concentrated under reduced pressure. The crude was purified by RP-HPLC [2-98% 0.1% TFA in water/ACN; Column: X Select Phenyl Hexyl (19*250) 5μ] to get the title Compound 961 (199 mg, 30%).

¹H NMR (400 MHz, DMSO-d₆): δ 12.44 (s, 1H), 10.26 (s, 1H), 8.94 (s, 1H), 8.67 (s, 2H), 8.29 (d, J=6.4 Hz, 1H), 8.24 (s, 1H), 7.88 (d, J=8.4 Hz, 2H), 7.25 (d, J=8.8 Hz, 2H), 7.16 (s, 1H), 6.23-6.09 (m, 2H), 5.64 (dd, J=9.6 Hz, 2.4 Hz, 1H), 4.53 (s, 2H), 4.21-4.13 (m, 1H), 3.92-3.88 (m, 4H), 3.78-3.75 (m, 4H), 3.61-3.50 (m, 2H), 3.10-2.90 (m, 1H), 2.82-2.76 (m, 1H), 1.90-1.81 (m, 3H).

LCMS=[M+H]⁺: 540.20; Purity=95.63%

### Example 107

### Compound 962 and Compound 952

### Preparation of 1-((S)-3-((S)-2,2,2-trifluoro-1-((4-(4-morpholino-7H-pyrrolo[2,3-d]pyrimidin-6-yl)phenyl)amino)ethyl)pyrrolidin-1-yl)prop-2-en-1-one (Compound 962) and 1-((S)-3-((R)-2,2,2-trifluoro-1-((4-(4-morpholino-7H-pyrrolo[2,3-d]pyrimidin-6-yl)phenyl)amino)ethyl)pyrrolidin-1-yl)prop-2-en-1-one (Compound 952)

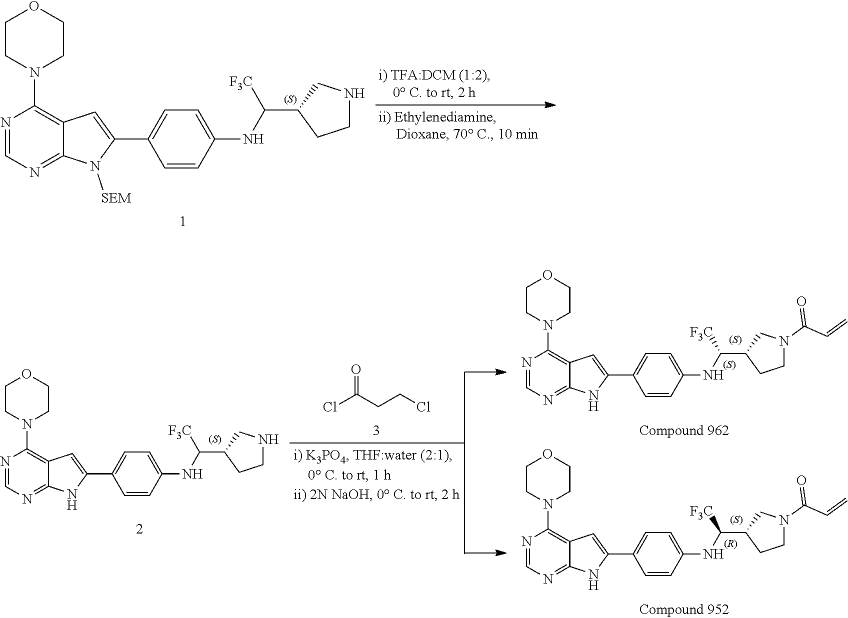

### Synthesis of 4-(4-morpholino-7H-pyrrolo[2,3-d]pyrimidin-6-yl)-N-(2,2,2-trifluoro-1-((S)-pyrrolidin-3-yl)ethyl)aniline (2)

To a stirred solution of {1-[(S)-3-pyrrolidinyl]-2,2,2-trifluoroethyl}[p-(4-morpholino-1-{[2-(trimethylsilyl) ethoxy]methyl}-1H-1,5,7-triazainden-2-yl)phenyl]amine 1 (780 mg, 1.35 mmol) in DCM (8 mL) was added TFA (4 mL) dropwise at 0° C. under N₂ atmosphere and stirred at RT for 2 h. After completion of reaction (monitored TLC), the reaction mixture was concentrated under reduced pressure, triturated with Et₂O and dried under reduced pressure. The crude was dissolved in 1,4-dioxane (6 mL) and was added 1,2-ethanediamine (0.3 mL, 4.06 mmol) at RT and stirred at 70° C. for 10 min. After completion of reaction (monitored by TLC), the reaction was poured into ice cold water (50 mL) and extracted with 25% IPA in CHCl₃ (3×80 mL). The combined organic layer was dried over Na₂SO₄, filtered and concentrated under reduced pressure to get the title compound as a brown solid 2 (450 mg; crude), which was used in next step directly without further purification.

LCMS=[M−H]: 444.18; Purity=58%.

### Synthesis of 1-((S)-3-((S)-2,2,2-trifluoro-1-((4-(4-morpholino-7H-pyrrolo[2,3-d]pyrimidin-6-yl)phenyl)amino)ethyl)pyrrolidin-1-yl)prop-2-en-1-one (Compound 962) and 1-((S)-3-((R)-2,2,2-trifluoro-1-((4-(4-morpholino-7H-pyrrolo[2,3-d]pyrimidin-6-yl)phenyl)amino)ethyl)pyrrolidin-1-yl)prop-2-en-1-one (Compound 952)

To an ice cold stirred solution of 4-(4-morpholino-7H-pyrrolo[2,3-d]pyrimidin-6-yl)-N-(2,2,2-trifluoro-1-((S)-pyrrolidin-3-yl)ethyl)aniline 2 (450 mg, 1.01 mmol) in THF (6 mL) was added K₃PO₄ (428 mg, 2.02 mmol) dissolved in water (5 mL) at 0° C. and stirred for 5 min. Then a solution of 3-chloropropanoyl chloride 3 (115 μL, 1.21 mmol) in THF (1 mL) was added drop wise at 0° C. and stirred at RT for 1 h. After completion of starting material (acylation reaction, monitored by TLC), 2N aq. NaOH (6.5 mL, 13 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [2-98% 0.1% TFA in water/ACN; Column: X Select CSH C18 (19*250) 10μ] to get the title Compound 962 (22 mg, 3%; Peak 1) and Compound 952 (45 mg, 6%; Peak 2) as a separable diastereomer [Note: Chirality arbitrarily assigned].

Analytical data of Compound 962: 1H NMR (400 MHz, DMSO-d₆): δ 12.45 (s, 1H), 8.24 (s, 1H), 7.68 (d, J=8.4 Hz, 2H), 7.07 (brs, 1H), 6.90-6.86 (m, 2H), 6.58-6.49 (m, 1H), 6.35 (d, J=10 Hz, 1H), 6.14-6.08 (m, 1H), 5.67-5.64 (m, 1H), 4.64-4.52 (m, 1H), 3.91-3.89 (m, 4H), 3.81-3.77 (m, 4H), 3.70-3.57 (m, 3H), 3.47-3.34 (m, 1H), 3.24-3.18 (m, 1H), 2.12-2.01 (m, 1H), 1.89-1.69 (m, 1H).

LCMS=[M+H]⁺: 501.23; Purity=96.01%.

Analytical data of Compound 952: 1H NMR (400 MHz, DMSO-d₆): δ 12.48 (s, 1H), 8.25 (m, 1H), 7.69 (d, J=8.4 Hz, 2H), 7.07 (brs, 1H), 6.87 (d, J=8.4 Hz, 2H), 6.59-6.46 (m, 1H), 6.38 (t, J=9.6 Hz, 1H), 6.14-6.08 (m, 1H), 5.67-5.61 (m, 1H), 4.60-4.52 (m, 1H), 3.91-3.88 (m, 4H), 3.81-3.78 (m, 4H), 3.75-3.69 (m, 1H), 3.55-3.47 (m, 1H), 3.38 (t, J=10.4 Hz, 1H), 3.32-3.26 (m, 1H), 3.14 (t, J=11.2 Hz, 1H), 2.11-2.01 (m, 1H), 1.92-1.75 (m, 1H).

LCMS=[M+H]⁺: 501.13; Purity=98.58%.

### Example 108

### Compound 963

### Preparation of 1-((3S,3′R)-3-(2,2,2-trifluoro-1-((4-(4-morpholino-7H-pyrrolo[2,3-d]pyrimidin-6-yl)phenyl)amino)ethyl)-[1,3′-bipyrrolidin]-1′-yl)prop-2-en-1-one (Compound 963)

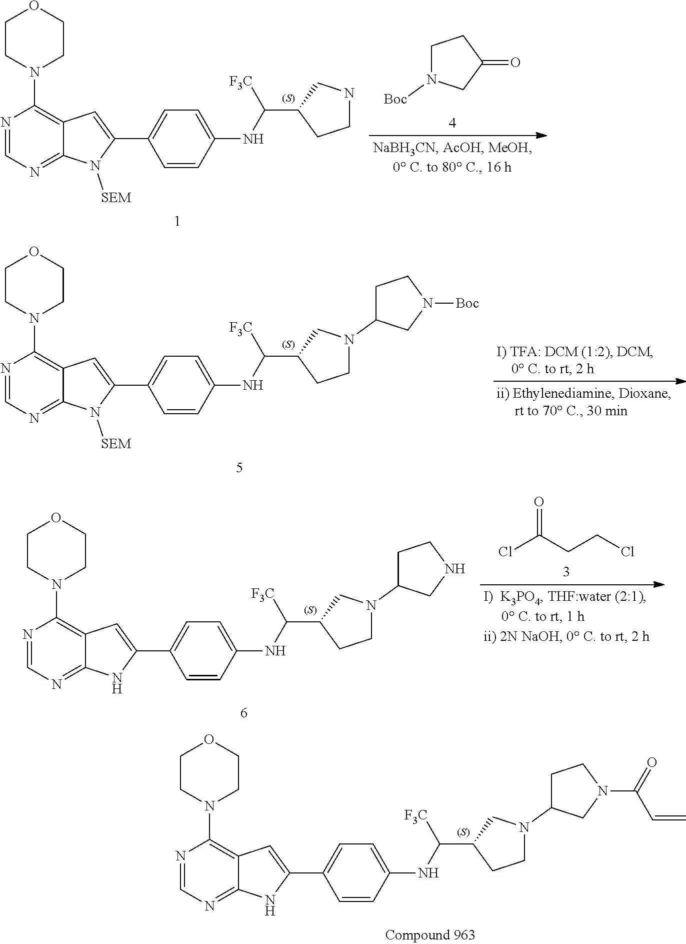

### Synthesis of tert-butyl (3S)-3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)-[1,3′-bipyrrolidine]-1′-carboxylate (5)

To a stirred solution of 4-(4-morpholino-7-((2-(trimethylsilyl)ethoxy)methyl)-7H-pyrrolo[2,3-d]pyrimidin-6-yl)-N-(2,2,2-trifluoro-1-((S)-pyrrolidin-3-yl)ethyl)aniline 1 (800 mg, 1.39 mmol) and tert-butyl 3-oxopyrrolidine-1-carboxylate 4 (385 mg, 2.08 mmol) in MeOH (10 mL) was added AcOH (160 μL, 2.77 mmol) at 0° C. The reaction mixture was stirred at 80° C. for 2 h. Then the reaction mixture was cooled to 0° C., and a solution of NaCNBH₃ (166 mg, 2.77 mmol) in MeOH (1 mL) was added and stirred at 80° C. for 16 h. After completion of reaction (monitored by TLC and LCMS), the reaction mixture was quenched with sat. NaHCO₃ (20 mL) and extracted with DCM (3×80 mL). The combined organic layer was dried over Na₂SO₄, filtered and concentrated under reduced pressure. The crude was purified by flash chromatography (silica gel 100-200 M, 12 g SNAP) using eluents 3-5% MeOH in DCM to get the title compound as a white solid 5 (80 mg, 80%) LCMS=[M+H]⁺: 746.41; Purity=75%.

### Synthesis of N-(1-((3S)-[1,3′-bipyrrolidin]-3-yl)-2,2,2-trifluoroethyl)-4-(4-morpholino-7H-pyrrolo[2,3-d]pyrimidin-6-yl)aniline (6)

To a stirred solution of tert-butyl (3S)-3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)-[1,3′-bipyrrolidine]-1′-carboxylate 5 (800 mg, 1.07 mmol) in DCM (6 mL) was added TFA (3 mL) dropwise at 0° C. under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 1,4-dioxane (5 mL) and ethylenediamine (230 μL, 3.22 mmol) was added and stirred at 70° C. for 30 min. After completion of reaction (monitored by LCMS), the reaction was poured into ice cold water (40 mL) and extracted with 25% IPA in CHCl₃ (3×50 mL). The combined organic layer was dried over Na₂SO₄, filtered and concentrated under reduced pressure to get the title compound as a brown solid 6 (600 mg; crude), which was used in next step directly without further purification.

LCMS=[M+H]: 516.30; Purity=69%.

### Synthesis of 1-((3S,3′R)-3-(2,2,2-trifluoro-1-((4-(4-morpholino-7H-pyrrolo[2,3-d]pyrimidin-6-yl)phenyl)amino)ethyl)-[1,3′-bipyrrolidin]-1′-yl)prop-2-en-1-one (Compound 963)

To an ice cold stirred solution of N-(1-((3S)-[1,3′-bipyrrolidin]-3-yl)-2,2,2-trifluoroethyl)-4-(4-morpholino-7H-pyrrolo[2,3-d]pyrimidin-6-yl)aniline 6 (500 mg, 0.970 mmol) in THF (6 mL) was added K₃PO₄ (412 mg, 1.94 mmol) dissolved in water (3 mL) under N₂ atmosphere and stirred for 5 min. Then a solution of 3-chloropropionyl chloride 3 (140 μL, 1.45 mmol) in THF (1 mL) was added drop wise to the reaction mixture at 0° C. and stirred at RT for 1 h. After completion of starting material (acylation reaction, monitored by TLC), 2N aq. NaOH (5.5 mL, 11.6 mmol) was added to the reaction mixture at 0° C. and stirred at RT for 2 h. After completion of reaction (complete elimination, monitored by TLC and LCMS), the reaction mixture was diluted with water (40 mL) and extracted with 2-MeTHF (3×50 mL). The combined organic layer was dried over Na2SO₄, filtered and concentrated under reduced pressure. The crude was purified by RP-HPLC [5-95% 5 mM Ammonium bicarbonate in water/ACN; Column: X Select Phenyl Hexyl (19*250) 5μ] to get the title Compound 963 (37 mg, 7%). [Note: Chirality arbitrarily assigned].

¹H NMR (400 MHz, DMSO-d₆): δ 11.97 (s, 1H), 8.13 (s, 1H), 7.69-7.67 (m, 2H), 6.90-6.87 (m, 4H), 6.59-6.42 (m, 2H), 6.17-6.13 (m, 1H), 5.69 (brs, 1H), 4.70-4.65 (m, 1H), 4.34-4.25 (m, 2H), 3.99-3.83 (m, 4H), 3.75-3.69 (m, 5H), 3.58-3.54 (m, 4H), 3.12-2.90 (m, 1H), 2.81-2.62 (m, 1H), 2.32-2.24 (m, 2H), 2.12-1.77 (m, 2H).

LCMS=[M+H]⁺: 570.28; Purity=95.06%.

### Example 109

### Compound 964 and Compound 965

### Preparation of 1-((S)-3-(4-((R)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 964) and 1-((R)-3-(4-((R)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 965)

Chiral separation of Compound 939 resulted in both enantiomers 1-((S)-3-(4-((R)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 964) and 1-((R)-3-(4-((R)-2,2,2-trifluoro-1-((4-(4-morpholino-7H-pyrrolo[2,3-d]pyrimidin-6-yl)phenyl)amino)ethyl)piperidin-1-yl)pyrrolidin-1-yl)prop-2-en-1-one (Compound 965).

Note: Chirality arbitrarily assigned

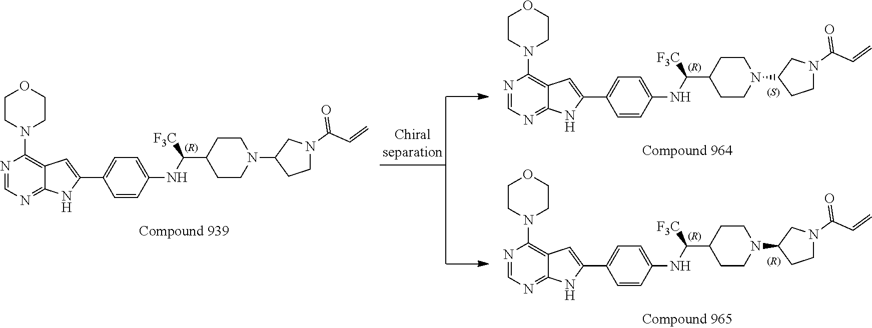

Methods for SFC purification:

Column: CHIRALCEL OJ-H (30 mm)

Mobile Phase: i) CO₂
ii) 0.2% Et3N in ACN: MeOH (1:1)

Flow rate: Isocratic 100 mL/Min, wavelength: 265 nm and 329 nm

Analytical data of Compound 964:

¹H NMR (400 MHz, DMSO-d₆): δ 11.94 (s, 1H), 8.12 (s, 1H), 7.63 (d, J=8.8 Hz, 2H), 6.87-6.83 (m, 3H), 6.61-6.50 (m, 1H), 6.31-6.06 (m, 2H), 5.66-5.61 (m, 1H), 4.27-4.25 (m, 1H), 3.85-3.82 (m, 4H), 3.74-3.65 (m, 6H), 3.20-3.18 (m, 2H), 3.00-2.88 (m, 2H), 2.82-2.76 (m, 2H), 2.22-2.15 (m, 1H), 1.99-1.90 (m, 2H), 1.78-1.74 (m, 1H), 1.70-1.67 (m, 2H), 1.50-1.46 (m, 2H).

LCMS=[M+H]⁺: 584.37; Purity=98.37%.

Analytical data of Compound 965:

¹H NMR (400 MHz, DMSO-d₆): δ 11.95 (s, 1H), 8.13 (s, 1H), 7.64 (d, J=8.4 Hz, 2H), 6.88-6.84 (m, 3H), 6.60-6.51 (m, 1H), 6.13-6.06 (m, 2H), 5.66-5.61 (m, 1H), 4.37-4.28 (m, 1H), 3.85-3.80 (m, 4H), 3.74-3.72 (m, 4H), 3.70-3.66 (m, 1H), 3.58-3.53 (m, 1H), 3.46-3.39 (m, 1H), 3.23-3.18 (m, 1H), 2.99-2.97 (m, 1H), 2.88-2.75 (m, 2H), 2.17-2.10 (m, 1H), 2.06-1.90 (m, 2H), 1.77-1.59 (m, 4H), 1.57-1.41 (m, 2H).

LCMS=[M+H]⁺: 584.36; Purity=98.10%.

### Example 110

### Compound 966

### Preparation of (R)—N-(3-methyl-1-((5-((4-(4-morpholino-7H-pyrrolo[2,3-d]pyrimidin-6-yl)phenyl)amino)pyrimidin-2-yl)methyl)piperidin-3-yl)acrylamide (Compound 966)

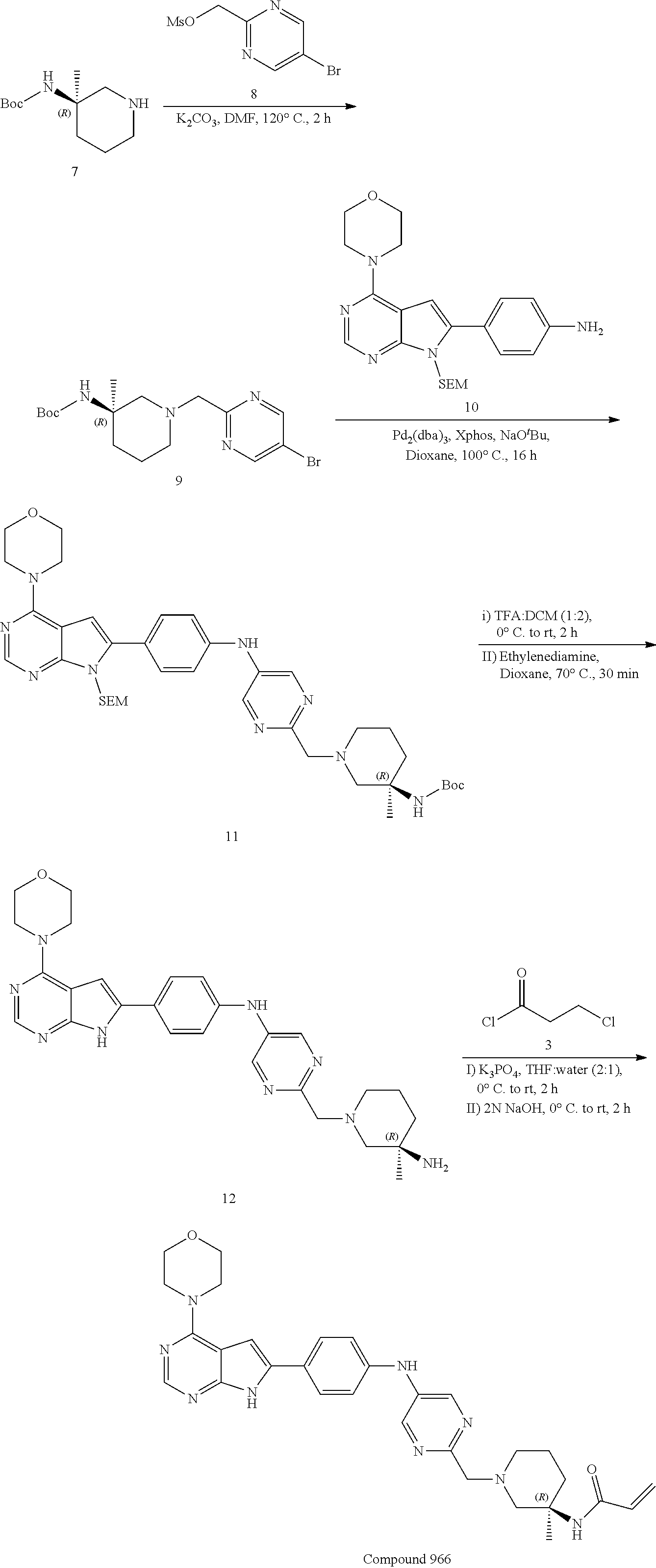

### Synthesis of tert-butyl (R)-(1-((5-bromopyrimidin-2-yl)methyl)-3-methylpiperidin-3-yl)carbamate (9)

To a stirred solution of tert-butyl (R)-(3-methylpiperidin-3-yl)carbamate 7 (250 mg, 1.17 mmol) in DMF (5 mL) was added K₂CO₃ (806 mg, 5.83 mmol) and (5-bromopyrimidin-2-yl)methyl methanesulfonate 8 (623 mg, 2.33 mmol) at 0° C. under N₂ atmosphere. The reaction mixture was stirred at 120° C. for 2 h. After completion of reaction (monitored by TLC), the reaction mixture was poured into ice cold water (50 mL) and extracted with EtOAc (3×80 mL). The combined organic layer was dried over Na₂SO₄, filtered and concentrated under reduced pressure. The crude was purified by flash column chromatography (silica gel 100-200 M, 12 g SNAP) using eluents 20-30% EtOAc in heptane to get the title compound as a brown solid 9 (350 mg, 78%).

LCMS=[M+H]⁺: 385.27; Purity=70%.

### Synthesis of tert-butyl (R)-(3-methyl-1-((5-((4-(4-morpholino-7-((2-(trimethylsilyl)ethoxy)methyl)-7H-pyrrolo[2,3-d]pyrimidin-6-yl)phenyl)amino)pyrimidin-2-yl)methyl)piperidin-3-yl)carbamate (11)

To a stirred solution of tert-butyl (R)-(1-((5-bromopyrimidin-2-yl)methyl)-3-methylpiperidin-3-yl)carbamate 9 (200 mg, 0.519 mmol) and 4-(4-morpholino-7-((2-(trimethylsilyl)ethoxy)methyl)-7H-pyrrolo[2,3-d]pyrimidin-6-yl)aniline 10 (221 mg, 0.519 mmol) in 1,4-dioxane (8 mL) was added NaO^{t}Bu (75 mg, 0.78 mmol) at RT. The reaction mixture was degassed with N₂ gas for 10 min, followed by Pd₂(dba)₃ (42 mg, 0.051 mmol) and XPhos (24.7 mg, 0.051 mmol) were added and stirred at 100° C. for 16 h. After completion of reaction (monitored by TLC and LCMS), the reaction mixture was diluted with water (50 mL) and extracted with EtOAc (3×80 mL). The combined organic layer was dried over Na₂SO₄, filtered and concentrated under reduced pressure. The crude was purified by flash column chromatography (silica gel 100-200 M, 12 g SNAP) using eluents 25-35% EtOAc in heptane to get the title compound as a brown solid 11 (400 mg, 64%).

LCMS=[M+H]⁺: 730.42; Purity=65%.

### Synthesis of (R)-2-((3-amino-3-methylpiperidin-1-yl)methyl)-N-(4-(4-morpholino-7H-pyrrolo[2,3-d]pyrimidin-6-yl)phenyl)pyrimidin-5-amine (12)

To a stirred solution of (R)-2-((3-amino-3-methylpiperidin-1-yl)methyl)-N-(4-(4-morpholino-7H pyrrolo[2,3-d]pyrimidin-6-yl)phenyl)pyrimidin-5-amine 11 (400 mg, 0.548 mmol) in DCM (5 mL) was added TFA (2.5 mL) dropwise at 0° C. under N₂ atmosphere and stirred at RT for 2 h. After completion of reaction (monitored by TLC), the reaction mixture was concentrated under reduced pressure, triturated with Et₂O and dried under reduced pressure. The crude was dissolved in 1,4-dioxane (5 mL) and ethylenediamine (73 μL, 1.1 mmol) was added to the reaction mixture and stirred at RT for 30 min. After completion of reaction (monitored by LCMS), the reaction was poured into ice cold water (50 mL) and extracted with 25% IPA in CHCl₃ (3×50 mL). The combined organic layer was dried over Na₂SO₄, filtered and concentrated under reduced pressure to get the title compound as a brown solid 12 (300 mg; crude), which was used in next step directly without further purification.

LCMS=[M−H]: 498.46; Purity=58%.

### Synthesis of (R)—N-(3-methyl-1-((5-((4-(4-morpholino-7H-pyrrolo[2,3-d]pyrimidin-6-yl)phenyl)amino)pyrimidin-2-yl)methyl)piperidin-3-yl)acrylamide (Compound 966)

To a stirred solution of tert-butyl (R)-(3-methyl-1-((5-((4-(4-morpholino-7-((2-(trimethylsilyl)ethoxy)methyl)-7H-pyrrolo[2,3-d]pyrimidin-6-yl)phenyl)amino)pyrimidin-2-yl)methyl)piperidin-3-yl)carbamate 12 (250 mg, 0.5 mmol) in THF (5 mL) was added K₃PO₄ (212 mg, 1 mmol) dissolved in water (2.5 mL) at 0° C. under N₂ atmosphere and stirred for 5 min. Then a solution of 3-chloropropionyl chloride 3 (72 μL, 1.8 mmol) in THF (1 mL) was added drop wise to the reaction mixture at 0° C. and stirred at RT for 1 h. After completion of starting material (acylation reaction, monitored by TLC), 2N aq. NaOH (3 mL, 6 mmol) was added to the reaction mixture at 0° C. and stirred at RT for 2 h. After completion of reaction (complete elimination, monitored by TLC and LCMS), the reaction mixture was diluted with water (80 mL) and extracted with 2-MeTHF (3×80 mL). The combined organic layer was dried over Na₂SO₄, filtered and concentrated under reduced pressure. The crude was purified RP-HPLC to get the title Compound 966 (17 mg, 5%).

¹H NMR (400 MHz, DMSO-d₆): δ 12.40 (s, 1H), 9.54 (brs, 1H), 8.95 (s, 1H), 8.62 (s, 2H), 8.22 (s, 1H), 7.89 (d, J=8.8 Hz, 2H), 7.25 (d, J=8.8 Hz, 2H), 7.08 (brs, 1H), 6.37-6.30 (m, 1H), 6.16-6.11 (m, 1H), 5.66-5.64 (m, 1H), 4.46-4.42 (m, 1H), 4.36-4.33 (m, 1H), 4.17-4.14 (m, 1H), 3.91-3.89 (m, 4H), 3.78-3.75 (m, 4H), 3.52-3.46 (m, 1H), 3.05-2.86 (m, 2H), 2.08-2.05 (m, 1H), 1.92-1.88 (m, 1H), 1.79-1.76 (m, 1H), 1.52-1.47 (m, 1H), 1.23 (s, 3H).

LCMS=[M+H]⁺: 554.39; Purity=97%.

### Example 111

### Compound 967

### Preparation of 1-(4-((R)-3-((S)-2,2,2-trifluoro-1-((4-(4-morpholino-7H-pyrrolo[2,3-d]pyrimidin-6-yl)phenyl)amino)ethyl)pyrrolidin-1-yl)piperidin-1-yl)prop-2-en-1-one (Compound 967)

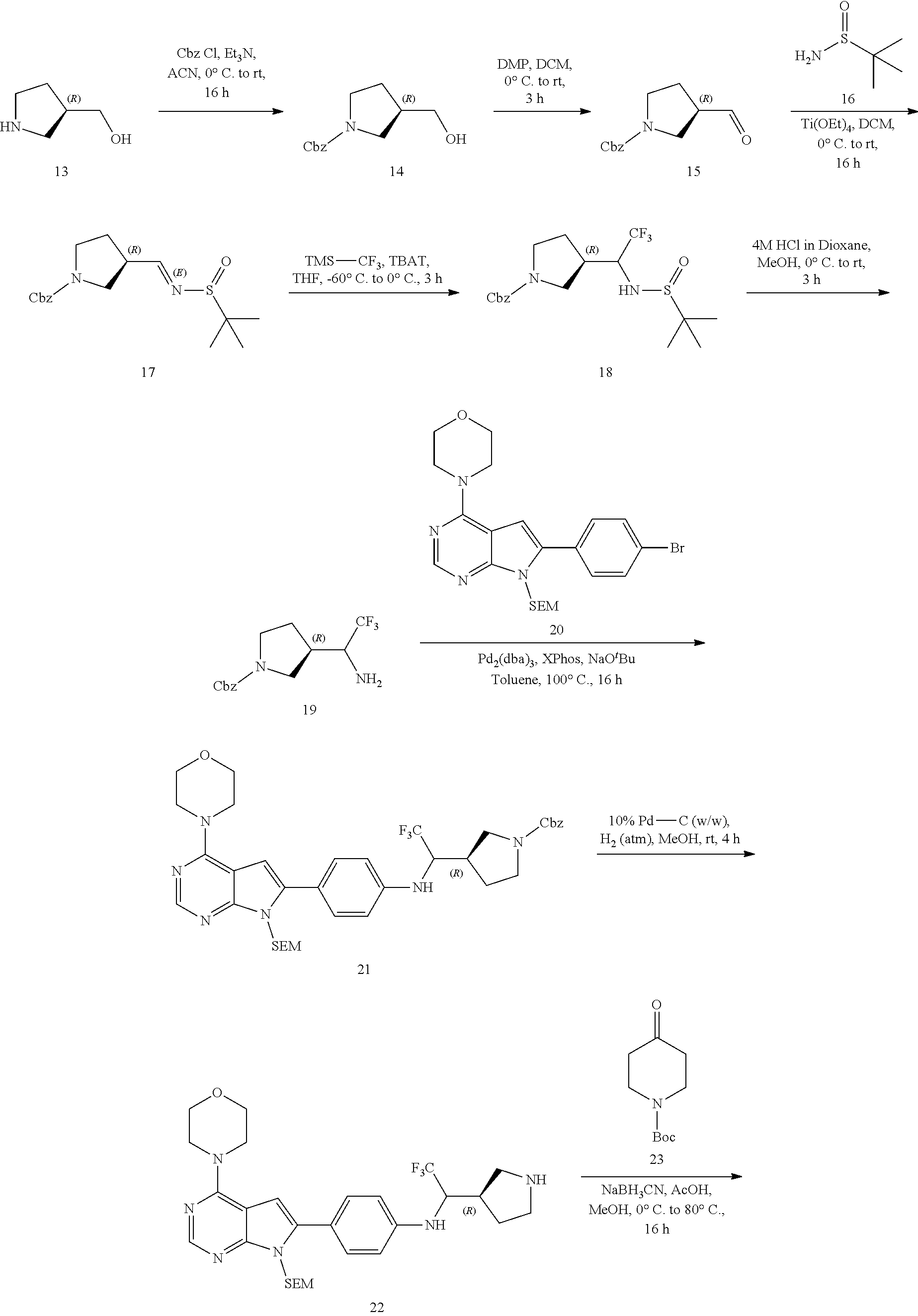

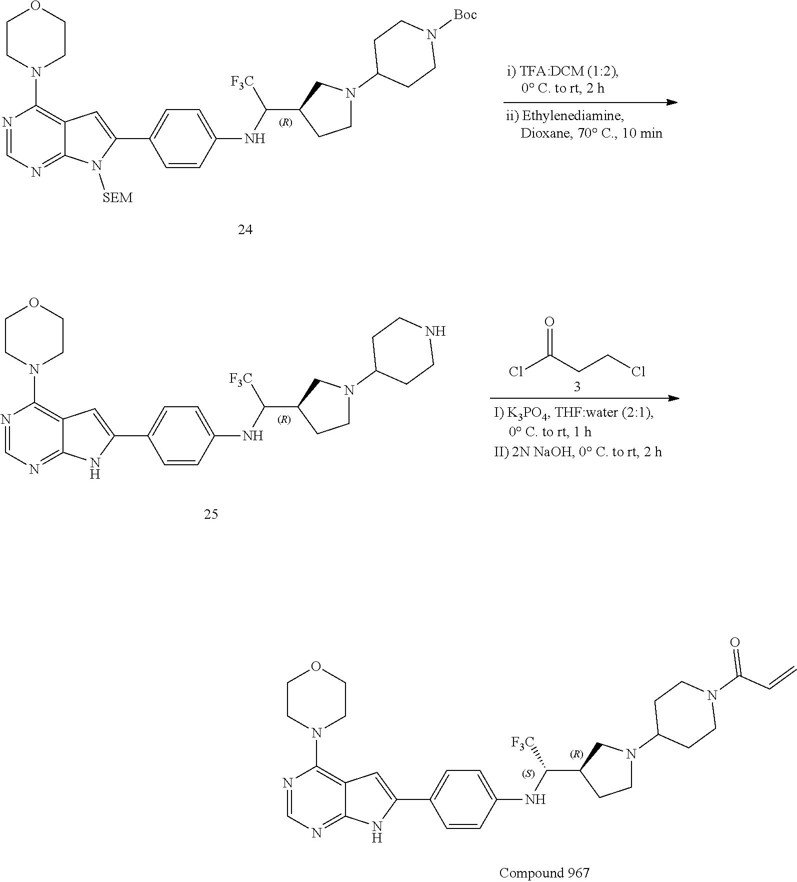

### Synthesis of benzyl (R)-3-(hydroxymethyl)pyrrolidine-1-carboxylate (14)

To a stirred solution of (R)-pyrrolidin-3-ylmethanol 13 (5 g, 49.4 mmol) in ACN (30 mL) was added Et3N (7.6 mL, 54.4 mmol) at 0° C. under N₂ atmosphere, followed by Cbz-Cl (15.5 mL, 54.4 mmol) was added to the reaction mixture at 0° C. and stirred at RT for 16 h. After completion of reaction (monitored by TLC), the reaction mixture was diluted with water (100 mL) and extracted with EtOAc (3×100 mL). The combined organic layer was washed with brine (2×100 mL), dried over Na₂SO₄ and concentrated under reduced pressure. The crude was purified by flash column chromatography (silica gel 100-200M, 40 g SNAP) using eluents 40-50% EtOAc in heptane to get the title compound as a colorless sticky gel 14 (5 g, 40%).

LCMS=[M+H]⁺: 236.14; Purity=94%.

### Synthesis of Benzyl (R)-3-formylpyrrolidine-1-carboxylate (15)

To a stirred solution of benzyl (R)-3-(hydroxymethyl)pyrrolidine-1-carboxylate 14 (5 g, 21.3 mmol) in DCM (50 mL) was added DMP (10.8 g, 25.5 mmol) portion wise at 0° C. under N₂ atmosphere and stirred at RT for 3 h. After completion of reaction (monitored by TLC), the reaction mixture was quenched with sat. Na2S203 (100 mL) and sat. NaHCO₃ (100 mL) and stirred at RT for 15 min, till it became clear solution. The reaction mixture was extracted with DCM (2×100 mL). The combined organic layer was washed with sat. NaHCO₃ (2×50 mL), dried over Na₂SO₄, filtered and concentrated under reduced pressure to get the title compound as a yellow sticky gel 15 (4.5 g; crude), which was used in next step without further purification.

¹H NMR (400 MHz, DMSO-d₆): δ 9.62 (s, 1H), 7.36-7.25 (m, 5H), 5.07 (s, 2H), 3.68-3.62 (m, 1H), 3.47-3.40 (m, 2H), 3.29-3.20 (m, 2H), 2.14-2.09 (m, 2H).

### Synthesis of Benzyl (3R)-3-((E)-((tert-butylsulfinyl)imino)methyl)pyrrolidine-1-carboxylate (17)

To a stirred solution of benzyl (R)-3-formylpyrrolidine-1-carboxylate 15 (4.5 g, 19.3 mmol) in DCM (50 mL) was added 2-methylpropane-2-sulfinamide 16 (2.81 g, 23.1 mmol) at 0° C. under N₂ atmosphere, followed by Ti (OEt) 4 (8.2 mL, 38.6 mmol). The reaction mixture was stirred at RT for 16 h. After completion of reaction (monitored by TLC), the reaction mixture was quenched with brine solution (100 mL), filtered through celite pad and washed with DCM (100 mL). The filtrate was extracted with DCM (3×100 mL). The combined organic layer was dried over Na₂SO₄, filtered and evaporated under reduced pressure. The crude was purified by flash column chromatography (silica gel 100-200 M, 40 g SNAP) using eluents 30-40% EtOAc in heptane to get the title compound as a colorless sticky solid 17 (3.3 g, 46%).

LCMS=[M+H]⁺: 337.18; Purity=94%.

### Synthesis of Benzyl (3R)-3-(1-((tert-butylsulfinyl)amino)-2,2,2-trifluoroethyl)pyrrolidine-1-carboxylate (18)

To a stirred solution of benzyl (3R)-3-((E)-((tert-butylsulfinyl)imino)methyl)pyrrolidine-1-carboxylate 17 (3.3 g, 9.81 mmol) in dry THF (50 mL) was added TBAT (5.82 g, 10.8 mmol) under N₂ atmosphere. The reaction mixture was cooled at −60° C., and a solution of TMS-CF₃ (2.9 mL, 19.6 mmol) dissolved in dry THF (10 mL) was added slowly and stirred for 1 h. Then the reaction mixture was warmed to 0° C. and stirred for 3 h. After completion of reaction (monitored by TLC), the reaction mixture was quenched with ice cold water (100 mL) and extracted with EtOAc (3×100 mL). The combined organic layer was dried over Na₂SO₄, filtered and evaporated under reduced pressure. The crude was purified by flash chromatography (silica gel 100-200 M, 40 g SNAP) using eluents 25-30% EtOAc in heptane to get the title compound as colorless sticky oil 18 (2.8 g, 61%) as a 1:1 mixture of diastereomers.

LCMS=[M+H]⁺: 407.20; Purity=87%.

### Synthesis of Benzyl (3R)-3-(1-amino-2,2,2-trifluoroethyl)pyrrolidine-1-carboxylate (19)

To a stirred solution of benzyl (3R)-3-(1-((tert-butylsulfinyl)amino)-2,2,2-trifluoroethyl)pyrrolidine-1-carboxylate 18 (2.8 g, 12.1 mmol) in MeOH (50 mL) was added 4N HCl in 1,4-dioxane (17.5 mL, 68.9 mmol) dropwise at 0° C. under N₂ atmosphere. The reaction mixture was stirred at RT for 3 h. After completion of reaction (monitored by TLC), the reaction mixture was concentrated under reduced pressure. The crude was basified with sat. NaHCO₃ to pH~7-8 and extracted with 25% IPA in CHCl₃ (3×80 mL). The combined organic was washed with brine (2×50 mL), dried over Na₂SO₄, filtered and concentrated under reduced pressure to get the title compound as an orange sticky solid 19 (2 g; crude), which was used as such in next step without purification.

LCMS=[M+H]⁺: 303.16; Purity=79%.

### Synthesis of Benzyl (3R)-3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)pyrrolidine-1-carboxylate (21)

To a stirred solution of benzyl (3R)-3-(1-amino-2,2,2-trifluoroethyl)pyrrolidine-1-carboxylate 19 (2 g, 6.67 mmol) and 4-(6-(4-bromophenyl)-7-((2-(trimethylsilyl)ethoxy)methyl)-7H-pyrrolo[2,3-d]pyrimidin-4-yl)morpholine 20 (2.2 g, 4.5 mmol) in toluene (20 mL) was added Cs₂CO₃ (2.92 g, 8.98 mmol) at RT. The reaction mixture was degassed with N₂ gas for 10 min, followed by RuPhos (210 mg, 0.450 mmol) and Pd(OAc)₂ (50.2 mg, 0.224 mmol) were added and stirred at 100° C. for 4 h. After completion of reaction (monitored by TLC and LCMS), the reaction mixture was diluted with water (50 mL) and extracted with EtOAc (3×80 mL). The combined organic layer was washed with brine (2×50 mL), dried over Na₂SO₄, filtered and concentrated under reduced pressure. The crude was purified by flash column chromatography (silica gel 100-200 M, 12 g SNAP) using eluents 30-40% EtOAc in heptane to get the title compound as a yellow sticky solid 21 (1.8 g, 42%).

LCMS=[M+H]⁺: 711.33; Purity=74%.

### Synthesis of 4-(4-morpholino-7-((2-(trimethylsilyl)ethoxy)methyl)-7H-pyrrolo[2,3-d]pyrimidin-6-yl)-N-(2,2,2-trifluoro-1-((R)-pyrrolidin-3-yl)ethyl)aniline (22)

To a solution benzyl (3R)-3-(2,2,2-trifluoro-1-((4-(4-morpholino-7-((2-stirred of (trimethylsilyl)ethoxy)methyl)-7H-pyrrolo[2,3-d]pyrimidin-6-yl)phenyl)amino)ethyl)pyrrolidine-1-carboxylate 21 (1.8 g, 2.53 mmol) in MeOH (10 mL) was added 10% Pd—C(1.9 g) at RT under H₂ atmosphere and stirred at RT for 4 h. After completion of reaction (monitored by TLC and LCMS), the reaction mixture was filtered through celite pad and washed with MeOH (50 mL). The filtrate was concentrated under reduced pressure to get the title compound as a yellow solid 22 (1.2 g; crude), which was used in next step without further purification.

LCMS=[M+H]⁺: 577.31; Purity=55%.

### Synthesis of tert-butyl 4-((3R)-3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)pyrrolidin-1-yl)piperidine-1-carboxylate (24)

To a stirred solution of 4-(4-morpholino-7-((2-(trimethylsilyl)ethoxy)methyl)-7H-pyrrolo[2,3-d]pyrimidin-6-yl)-N-(2,2,2-trifluoro-1-((R)-pyrrolidin-3-yl)ethyl)aniline 22 (1.2 g, 2.08 mmol) and tert-butyl 4-oxopiperidine-1-carboxylate 23 (622 mg, 3.12 mmol) in MeOH (10 mL) was added AcOH (238 μL, 4.16 mmol) at 0° C. and stirred at RT for 2 h. Then the reaction mixture was again cooled to 0° C. and NaCNBH₃ (249 mg, 4.16 mmol) was added. The reaction mixture was stirred at 80° C. for 16 h. After completion of reaction (monitored by TLC and LCMS), the reaction mixture was evaporated under reduced pressure. The crude was dissolved in 25% IPA in CHCl₃ (60 mL) and washed brine (2×50 mL). The organic layer was dried over Na2SO₄, filtered and concentrated under reduced pressure. The crude was purified by flash column chromatography (silica gel 100-200 M, 12 g SNAP) using eluents 3-5% MeOH in DCM to get the title compound as an off white solid 24 (1 g, 40%).

LCMS=[M+H]⁺: 760.40; Purity=64%.

### Synthesis of 4-(4-morpholino-7H-pyrrolo[2,3-d]pyrimidin-6-yl)-N-(2,2,2-trifluoro-1-((R)-1-(piperidin-4-yl)pyrrolidin-3-yl)ethyl)aniline (25)

To a stirred solution of tert-butyl 4-((3R)-3-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)pyrrolidin-1-yl)piperidine-1-carboxylate 24 (1 g, 1.32 mmol) in DCM (10 mL) was added TFA (5 mL) dropwise at 0° C. under N₂ atmosphere and stirred at RT for 2 h. After completion of reaction (monitored by TLC), the reaction mixture was concentrated under reduced pressure, triturated with Et₂O and dried over vacuum. The crude was dissolved in 1,4-dioxane (5 mL) and 1,2-ethanediamine (265 μL 3.95 mmol) was added and was stirred at 70° C. for 10 min. After completion of reaction (monitored by LCMS), the reaction mixture was diluted with water (10 mL) and extracted with 25% IPA in CHCl₃ (3×50 mL). The combined organic layer was dried over Na2SO₄, filtered and concentrated under reduced pressure to get the title compound as a yellow solid 25 (742 mg; crude), which was used in next step directly without further purification.

LCMS=[M+H]: 528.45; Purity=64%.

### Synthesis of 1-(4-((R)-3-((S)-2,2,2-trifluoro-1-((4-(4-morpholino-7H-pyrrolo[2,3-d]pyrimidin-6-yl)phenyl)amino)ethyl)pyrrolidin-1-yl)piperidin-1-yl)prop-2-en-1-one (Compound 967)

To a stirred solution of 4-(4-morpholino-7H-pyrrolo[2,3-d]pyrimidin-6-yl)-N-(2,2,2-trifluoro-1-((R)-1-(piperidin-4-yl)pyrrolidin-3-yl)ethyl)aniline 25 (742 mg, 1.4 mmol) in THF (10 mL) was added K₃PO₄ (595 mg, 2.8 mmol) dissolved in water (5 mL) at 0° C. and stirred for 5 min. Then a solution of 3-chloropropanoyl chloride 3 (213 mg, 1.68 mmol) in THF (1 mL) was added dropwise and stirred at RT for 1 h. After completion of starting material (acylation reaction, monitored by TLC), 2N aq. NaOH (8.5 mL, 16.8 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [2-98% 0.1% TFA in water/ACN; Column: X Select CSH C18 (19*250) 10μ] to get the title Compound 967 (14 mg, 2%) as a separable diastereomer. [Note: Chirality arbitrarily assigned]

¹H NMR (400 MHz, DMSO-d₆): δ 11.97 (s, 1H), 8.13 (s, 1H), 7.64 (d, J=8.4 Hz, 2H), 6.88 (brs, 1H), 6.81 (d, J=8.8 Hz, 2H), 6.78-6.71 (m, 1H), 6.19 (d, J=9.6 Hz, 1H), 6.07-6.02 (m, 1H), 5.62 (t, J=8.8 Hz, 1H), 4.35-4.29 (m, 1H), 4.19-4.16 (m, 1H), 3.99-3.90 (m, 1H), 3.85-3.83 (m, 4H), 3.74-3.72 (m, 4H), 3.08-3.05 (m, 1H), 2.87-2.67 (m, 4H), 2.36-2.33 (m, 1H), 2.27-2.20 (m, 1H), 1.96-1.90 (m, 1H), 1.79-1.77 (m, 2H), 1.70-1.68 (m, 1H), 1.25-1.23 (m, 2H). [1H may merged with solvent peak]

LCMS=[M+H]⁺: 584.32; Purity=99.55%.

### Example 112

### Compound 968

### Preparation of (E)-4-(azetidin-1-yl)-1-(4-(5-((4-(4-morpholino-1H-imidazo[4,5-c]pyridin-2-yl)phenyl)amino)pyrimidin-2-yl)piperazin-1-yl)but-2-en-1-one (Compound 968)

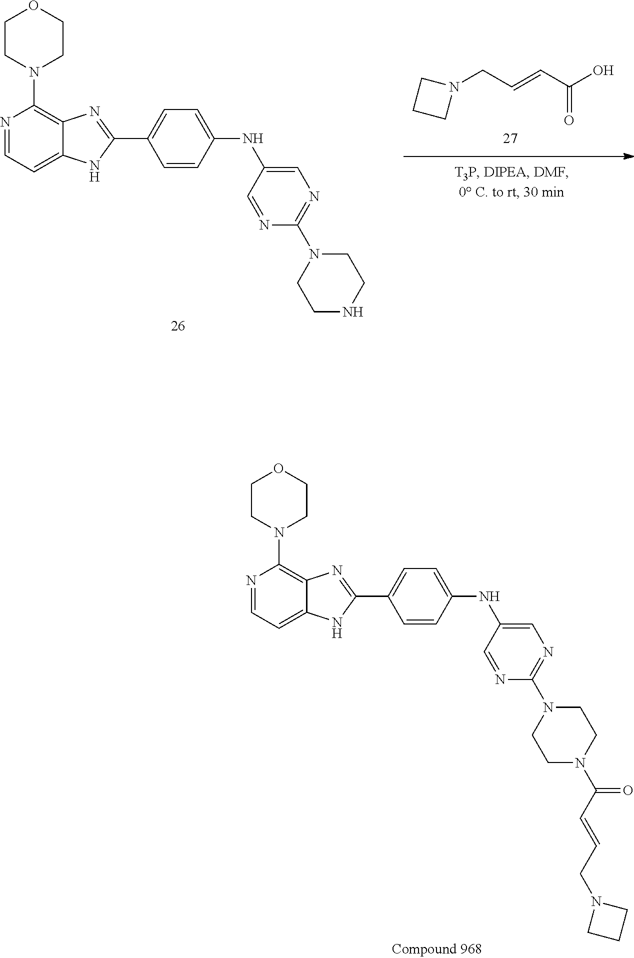 [SMILES: c1cc2[nH]c(-c3ccc(Nc4cnc(N5CCNCC5)nc4)cc3)nc2c(N2CCOCC2)n1 ; O=C(O)/C=C/CN1CCC1 ; O=C(/C=C/CN1CCC1)N1CCN(c2ncc(Nc3ccc(-c4nc5c(N6CCOCC6)nccc5[nH]4)cc3)cn2)CC1]

### Synthesis of (E)-4-(azetidin-1-yl)-1-(4-(5-((4-(4-morpholino-1H-imidazo[4,5-c]pyridin-2-yl)phenyl)amino)pyrimidin-2-yl)piperazin-1-yl)but-2-en-1-one (Compound 968)

To a stirred solution of N-(4-(4-morpholino-1H-imidazo[4,5-c]pyridin-2-yl)phenyl)-2-(piperazin-1-yl)pyrimidin-5-amine 26 (220 mg, 0.481 mmol) in DMF (5 mL) were added DIPEA (0.2 mL, 1.2 mmol), (E)-4-(azetidin-1-yl)but-2-enoic acid 27 (102 mg, 0.721 mmol) and T3P (0.4 mL, 0.625 mmol) sequentially at 0° C. under N₂ atmosphere. The reaction mixture was stirred at RT for 30 min. After completion of reaction (monitored by TLC), the reaction mixture was poured into ice cold water (50 mL) and extracted with EtOAc (3×50 mL). The combined organic layer was washed with ice cold brine (2×40 mL), dried over Na₂SO₄, filtered and concentrated under reduced pressure. The crude was purified by RP-HPLC [2-98% 0.1% TFA in Water/ACN; Column: X Select Fluoro Phenyl C18 (19*250) 5μ] to get the title Compound 968 (4 mg, 14%).

¹H NMR (400 MHz, DMSO-d₆): δ 13.73 (brs, 1H), 8.38 (s, 2H), 8.32 (s, 1H), 7.94 (d, J=8.8 Hz, 2H), 7.70 (d, J=6.4 Hz, 1H), 7.14-7.09 (m, 1H), 6.97-6.87 (m, 3H), 6.54-6.47 (m, 1H), 4.21-4.16 (m, 6H), 4.12-3.98 (m, 4H), 3.87-3.84 (m, 4H), 3.76-3.74 (m, 4H), 3.68-3.65 (m, 4H), 2.40-2.33 (m, 2H).

LCMS=[M+H]⁺: 581.39; Purity=92.25%.

### Example 113

### Compound 969

### Preparation of 1-(4-(5-((4-(4-(4,4-difluoropiperidin-1-yl)-7H-pyrrolo[2,3-d]pyrimidin-6-yl)phenyl)amino)pyrimidin-2-yl)piperazin-1-yl)prop-2-en-1-one (Compound 969)

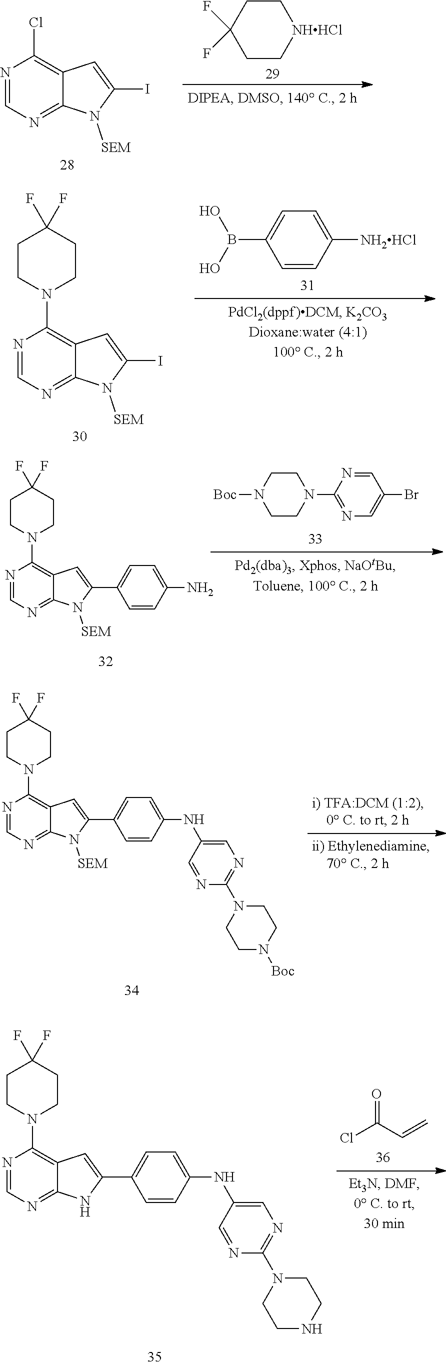

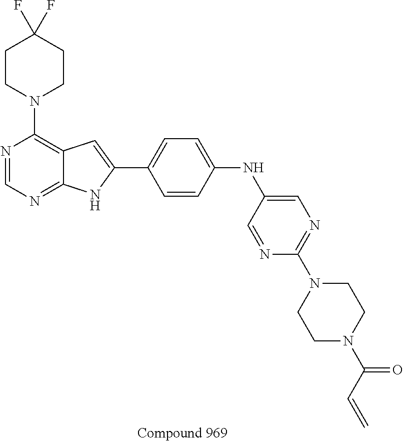 [SMILES: C=CC(=O)N1CCN(c2ncc(Nc3ccc(-c4cc5c(N6CCC(F)(F)CC6)ncnc5[nH]4)cc3)cn2)CC1]

### Synthesis of 4-(4,4-difluoropiperidin-1-yl)-6-iodo-7-((2-(trimethylsilyl)ethoxy)methyl)-7H-pyrrolo[2,3-d]pyrimidine (30)

To a stirred solution of 4-chloro-6-iodo-7-((2-(trimethylsilyl)ethoxy)methyl)-7H-pyrrolo[2,3-d]pyrimidine 28 (1 g, 2.44 mmol) and 4,4-difluoropiperidine hydrochloride 29 (385 mg, 2.44 mmol) in DMSO (10 mL) was added DIPEA (1.3 mL,7.32 mmol) at RT under N₂ atmosphere. The reaction mixture was stirred at 140° C. for 2 h. After completion of reaction (monitored by TLC), the reaction mixture was poured into ice cold water (50 mL). The obtained precipitate was filtered through sintered funnel, washed with Et₂O and dried under reduced pressure to get the title compound as a white solid 30 (1.3 g, 99%).

LCMS=[M+H]⁺: 495.09; Purity=94%.

### Synthesis of 4-(4-(4,4-difluoropiperidin-1-yl)-7-((2-(trimethylsilyl)ethoxy)methyl)-7H-pyrrolo[2,3-d]pyrimidin-6-yl)aniline (32)

To a stirred solution of 4-(4,4-difluoropiperidin-1-yl)-6-iodo-7-((2-(trimethylsilyl)ethoxy)methyl)-7H-pyrrolo[2,3-d]pyrimidine 30 (1.2 g, 2.43 mmol) and (4-aminophenyl) boronic acid hydrochloride 31 (842 mg, 4.85 mmol) in 1,4-dioxane (20 mL) was added K₂CO₃ (1 g, 7.28 mmol) dissolved in water (5 mL) at RT. The resulting reaction mixture was degassed with N₂ gas for 15 min, followed by PdCl₂(dppf)·DCM (198 mg, 0.243 mmol) was added to the reaction mixture and stirred at 100° C. for 2 h. After the completion of reaction (monitored by TLC and LCMS), the reaction mixture was filtered through celite pad and washed with EtOAc (100 mL). Then the filtrate was diluted with water (80 mL) and extracted with EtOAc (3×80 mL). The combined organic layer was dried over Na₂SO₄, filtered and evaporated under reduced pressure. The crude was purified by flash column chromatography (silica gel 100-200 M, 40 g SNAP) using eluents 25-30% EtOAc in heptane to get title compound as a brown solid 32 (900 mg, 80%).

LCMS=[M+H]⁺: 460.26; Purity=90%

### Synthesis of tert-butyl 4-(5-((4-(4-(4,4-difluoropiperidin-1-yl)-7-((2-(trimethylsilyl)ethoxy)methyl)-7H-pyrrolo[2,3-d]pyrimidin-6-yl)phenyl)amino)pyrimidin-2-yl)piperazine-1-carboxylate (34)

To a stirred solution of 4-(4-(4,4-difluoropiperidin-1-yl)-7-((2-(trimethylsilyl)ethoxy)methyl)-7H-pyrrolo[2,3-d]pyrimidin-6-yl)aniline 32 (800 mg, 1.69 mmol) and tert-butyl 4-(5-bromopyrimidin-2-yl)piperazine-1-carboxylate 33 (870 mg, 2.53 mmol) in toluene (10 mL) was added NaO^{t}Bu (487 mg, 5.07 mmol) at RT. The resulting reaction mixture was degassed with N₂ gas for 10 min, followed by XPhos (161 mg, 0.338 mmol) and Pd₂(dba)₃ (137 mg, 0.169 mmol) were added and stirred at 100° C. for 2 h. After completion of reaction (monitored by TLC and LCMS), the reaction mixture was diluted with water (80 mL) and extracted with EtOAc (3×80 mL). The combined organic layer was dried over Na₂SO₄, filtered and evaporated under reduced pressure. The crude was purified by flash column chromatography (silica gel 100-200 M, 40 g SNAP) using eluents 30-40% EtOAc in heptane to get the title compound as a yellow solid 34 (1.1 g, 47%).

LCMS=[M+H]⁺: 722.42; Purity=70%.

### Synthesis of N-(4-(4-(4,4-difluoropiperidin-1-yl)-7H-pyrrolo[2,3-d]pyrimidin-6-yl)phenyl)-2-(piperazin-1-yl)pyrimidin-5-amine (35)

To a stirred solution of tert-butyl 4-(5-((4-(4-(4,4-difluoropiperidin-1-yl)-7-((2-(trimethylsilyl)ethoxy)methyl)-7H-pyrrolo[2,3-d]pyrimidin-6-yl)phenyl)amino)pyrimidin-2-yl)piperazine-1-carboxylate 34 (1 g, 1.39 mmol) in DCM (10 mL) was added TFA (5 mL) dropwise at 0° C. under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 1,4-dioxane (8 mL) and ethylenediamine (0.3 mL, 4.16 mmol) was added and stirred at 70° C. for 30 min. After completion of reaction (monitored by LCMS), the reaction was poured into ice cold water (50 mL) and extracted with 25% IPA in CHCl₃ (3×60 mL). The combined organic layer was dried over Na₂SO₄, filtered and concentrated under reduced pressure to get the title compound as a yellow solid 35 (500 mg; crude), which was used in next step directly without further purification.

LCMS=[M+H]⁺: 492.27; Purity=65%.

### Synthesis of 1-(4-(5-((4-(4-(4,4-difluoropiperidin-1-yl)-7H-pyrrolo[2,3-d]pyrimidin-6-yl)phenyl)amino)pyrimidin-2-yl)piperazin-1-yl)prop-2-en-1-one (Compound 969)

To a stirred solution of N-(4-(4-(4,4-difluoropiperidin-1-yl)-7H-pyrrolo[2,3-d]pyrimidin-6-yl)phenyl)-2-(piperazin-1-yl)pyrimidin-5-amine 35 (500 mg, 1.02 mmol) in DMF (8 mL) was added Et₃N (0.4 mL, 3.05 mmol) at 0° C. under N₂ atmosphere and stirred for 5 min. Then a solution of acryloyl chloride 36 (100 μL, 1.22 mmol) in DMF (1 mL) was added dropwise to the reaction mixture at 0° C. The reaction mixture was stirred at RT for 30 min. After completion of reaction (monitored by TLC and LCMS), the reaction mixture was diluted with ice cold water (30 mL). The obtained precipitate was filtered through sintered funnel, washed with Et₂O and dried under reduced pressure. The crude was purified by RP-HPLC [5-95% 0.1% TFA in Water/ACN; Column: X Select CSH C18 (19*250) 10μ] to obtain the title Compound 969 (20 mg, 4%).

¹H NMR (400 MHz, DMSO-d₆): δ 12.43 (s, 1H), 8.34 (s, 2H), 8.25 (s, 1H), 8.01 (brs, 1H), 7.73 (d, J=8.4 Hz, 2H), 7.04 (brs, 1H), 6.85 (q, J=8 Hz, 3H), 6.15 (dd, J=16.8 Hz, 2.4 Hz, 1H), 5.72 (dd, J=10.4 Hz, 2.0 Hz, 1H), 4.04-4.01 (m, 4H), 3.77-3.71 (m, 4H), 3.67-3.64 (m, 4H), 2.17-2.10 (m, 4H).

LCMS=[M+H]⁺: 546.23; Purity=98.68%.

### Example 114

### Compound 970

### Preparation of 1-(4-(5-((4-(4-(1,1-dioxidothiomorpholino)-7H-pyrrolo[2,3-d]pyrimidin-6-yl)phenyl)amino)pyrimidin-2-yl)piperazin-1-yl)prop-2-en-1-one (Compound 970)

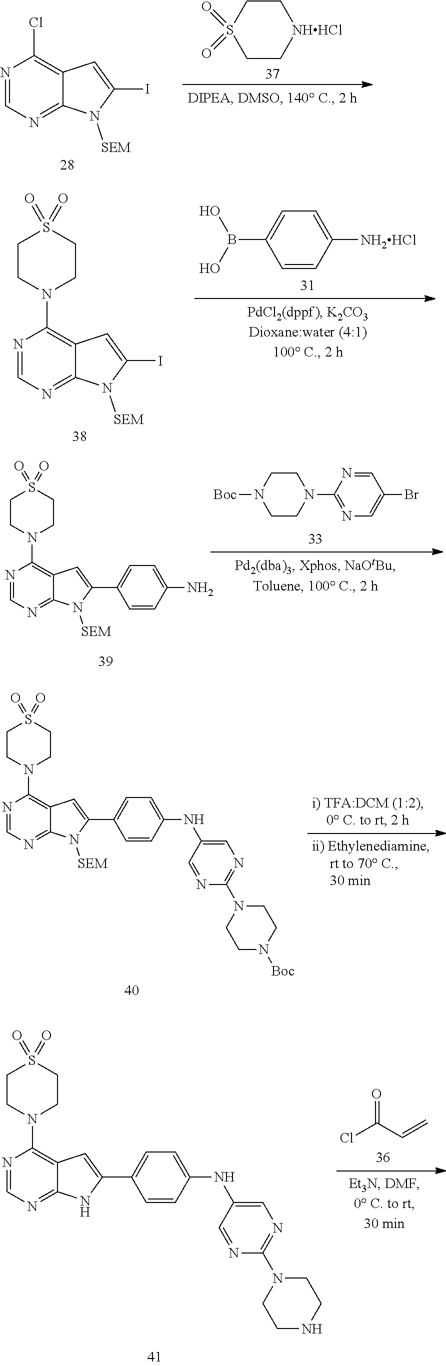

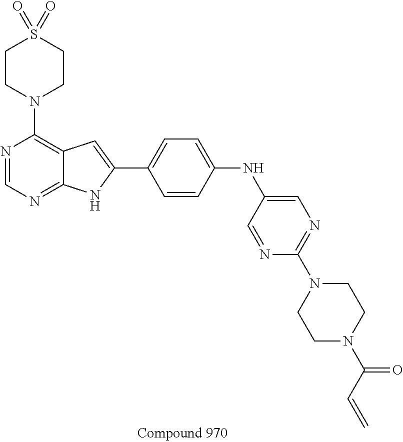 [SMILES: C=CC(=O)N1CCN(c2ncc(Nc3ccc(-c4cc5c(N6CCS(=O)(=O)CC6)ncnc5[nH]4)cc3)cn2)CC1]

### Synthesis of 4-(6-iodo-7-((2-(trimethylsilyl)ethoxy)methyl)-7H-pyrrolo[2,3-d]pyrimidin-4-yl)thiomorpholine 1,1-dioxide (38)

To a stirred solution of 4-chloro-6-iodo-7-((2-(trimethylsilyl)ethoxy)methyl)-7H-pyrrolo[2,3-d]pyrimidine 28 (1 g, 2.44 mmol) and thiomorpholine 1,1-dioxide hydrochloride 37 (419 mg, 2.44 mmol) in DMSO (15 mL) was added DIPEA (1.3 mL, 7.32 mmol) at RT under N₂ atmosphere. The reaction mixture was stirred at 140° C. for 2 h. After completion of reaction (monitored by TLC), the reaction mixture was poured into ice cold water (50 mL). The obtained precipitate was filtered through sintered funnel, washed with Et₂O and dried under reduced pressure to get the title compound as a white solid 38 (1.3 g, 89%).

LCMS=[M+H]⁺: 509.08; Purity=92%.

### Synthesis of 4-(6-(4-aminophenyl)-7-((2-(trimethylsilyl)ethoxy)methyl)-7H-pyrrolo[2,3-d]pyrimidin-4-yl)thiomorpholine 1,1-dioxide (39)

To a stirred solution of 4-(6-iodo-7-((2-(trimethylsilyl)ethoxy)methyl)-7H-pyrrolo[2,3-d]pyrimidin-4-yl)thiomorpholine 1,1-dioxide 38 (1.25 g, 2.46 mmol) and (4-aminophenyl) boronic acid hydrochloride 31 (853 mg, 4.92 mmol) in 1,4-dioxane (20 mL) was added K₂CO₃ (1 g, 7.33 mmol) dissolved in water (5 mL) at RT. The resulting reaction mixture was degassed with N₂ gas for 15 min, followed by PdCl₂(dppf)(201 mg, 0.246 mmol) was added to the reaction mixture and stirred at 100° C. for 2 h. After the completion of reaction (monitored by TLC and LCMS), the reaction mixture was filtered through celite pad and washed with EtOAc (100 mL). Then the filtrate was diluted with water (80 mL) and extracted with EtOAc (3×80 mL). The combined organic layer was dried over Na₂SO₄, filtered and evaporated under reduced pressure. The crude was purified by flash column chromatography (silica gel 100-200 M, 40 g SNAP) using eluents 25-30% EtOAc in heptane to get title compound as a brown solid 39 (850 mg, 73%).

LCMS=[M+H]⁺: 474.25; Purity=95%.

### Synthesis of tert-butyl 4-(5-((4-(4-(1,1-dioxidothiomorpholino)-7-((2-(trimethylsilyl)ethoxy)methyl)-7H-pyrrolo[2,3-d]pyrimidin-6-yl)phenyl)amino)pyrimidin-2-yl)piperazine-1-carboxylate (40)

To a stirred solution of 4-(6-(4-aminophenyl)-7-((2-(trimethylsilyl)ethoxy)methyl)-7H-pyrrolo[2,3-d]pyrimidin-4-yl)thiomorpholine 1,1-dioxide 39 (800 mg, 1.69 mmol) and tert-butyl 4-(5-bromopyrimidin-2-yl)piperazine-1-carboxylate 33 (870 mg, 2.53 mmol) in toluene (10 mL) was added NaO^{t}Bu (487 mg, 5.07 mmol) at RT. The resulting reaction mixture was degassed with N₂ gas for 10 min, followed by XPhos (161 mg, 0.338 mmol) and Pd₂(dba)₃ (137 mg, 0.169 mmol) were added and stirred at 100° C. for 2 h. After completion of reaction (monitored by TLC and LCMS), the reaction mixture was diluted with water (50 mL) and extracted with EtOAc (3×70 mL). The combined organic layer was dried over Na₂SO₄, filtered and evaporated under reduced pressure. The crude was purified by flash column chromatography (silica gel 100-200 M, 40 g SNAP) using eluents 30-40% EtOAc in heptane to get the title compound as a yellow solid 40 (1.1 g, 35%).

LCMS=[M+H]⁺: 736.38; Purity=40%

### Synthesis of 4-(6-(4-((2-(piperazin-1-yl)pyrimidin-5-yl)amino)phenyl)-7H-pyrrolo[2,3-d]pyrimidin-4-yl)thiomorpholine 1,1-dioxide (41)

To a stirred solution of tert-butyl 4-(5-((4-(4-(1,1-dioxidothiomorpholino)-7-((2-(trimethylsilyl)ethoxy)methyl)-7H-pyrrolo[2,3-d]pyrimidin-6-yl)phenyl)amino)pyrimidin-2-yl)piperazine-1-carboxylate 40 (1 g, 1.36 mmol) in DCM (12 mL) was added TFA (6 mL) dropwise at 0° C. under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 1,4-dioxane (8 mL) and ethylenediamine (0.3 mL, 4.08 mmol) was added and stirred at 70° C. for 30 min. After completion of reaction (monitored by LCMS), the reaction was poured into ice cold water (50 mL) and extracted with 25% IPA in CHCl₃ (3×60 mL). The combined organic layer was dried over Na₂SO₄, filtered and concentrated under reduced pressure to get the title compound as a yellow solid 41 (600 mg; crude), which was used in next step directly without further purification.

LCMS=[M+H]⁺: 506.2; Purity=60%.

### Synthesis of 1-(4-(5-((4-(4-(4,4-difluoropiperidin-1-yl)-7H-pyrrolo[2,3-d]pyrimidin-6-yl)phenyl)amino)pyrimidin-2-yl)piperazin-1-yl)prop-2-en-1-one (Compound 970)

To a stirred solution of 4-(6-(4-((2-(piperazin-1-yl)pyrimidin-5-yl)amino)phenyl)-7H-pyrrolo[2,3-d]pyrimidin-4-yl)thiomorpholine 1,1-dioxide 41 (500 mg, 1.02 mmol) in DMF (8 mL) was added Et₃N (0.4 mL, 3.05 mmol) at 0° C. under N₂ atmosphere and stirred for 5 min. Then a solution of acryloyl chloride 36 (100 μL, 1.22 mmol) in DMF (1 mL) was added dropwise to the reaction mixture at 0° C. The reaction mixture was stirred at RT for 30 min. After completion of reaction (monitored by TLC and LCMS), the reaction mixture was diluted with ice cold water (30 mL). The obtained precipitate was filtered through sintered funnel, washed with Et₂O and dried under reduced pressure. The crude was purified by RP-HPLC [5-95% 0.1% TFA in Water/ACN; Column: X TERRA RP18 (19*250) 10μ] to obtain the title Compound 970 (28 mg, 5%).

¹H NMR (400 MHz, DMSO-d₆): δ 12.29 (brs, 1H), 8.34 (m, 2H), 8.24 (s, 1H), 8.00 (brs, 1H), 7.73 (d, J=8.8 Hz, 2H), 6.96-6.95 (m, 1H), 6.89-6.84 (m, 3H), 6.15 (dd, J=16.8 Hz, 2.4 Hz, 1H), 5.72 (dd, J=10.4 Hz, 2.4 Hz, 1H), 4.34-4.32 (m, 4H), 3.78-3.72 (m, 4H), 3.67-3.64 (m, 4H), 3.28-3.26 (m, 4H).

LCMS=[M+H]⁺: 560.38; Purity=98.71%.

### Example 115

### Compound 971

### Preparation of 4-methoxy-1-(4-(5-((4-(4-morpholino-7H-pyrrolo[2,3-d]pyrimidin-6-yl)phenyl)amino)pyrimidin-2-yl)piperazin-1-yl)but-2-yn-1-one (Compound 971)

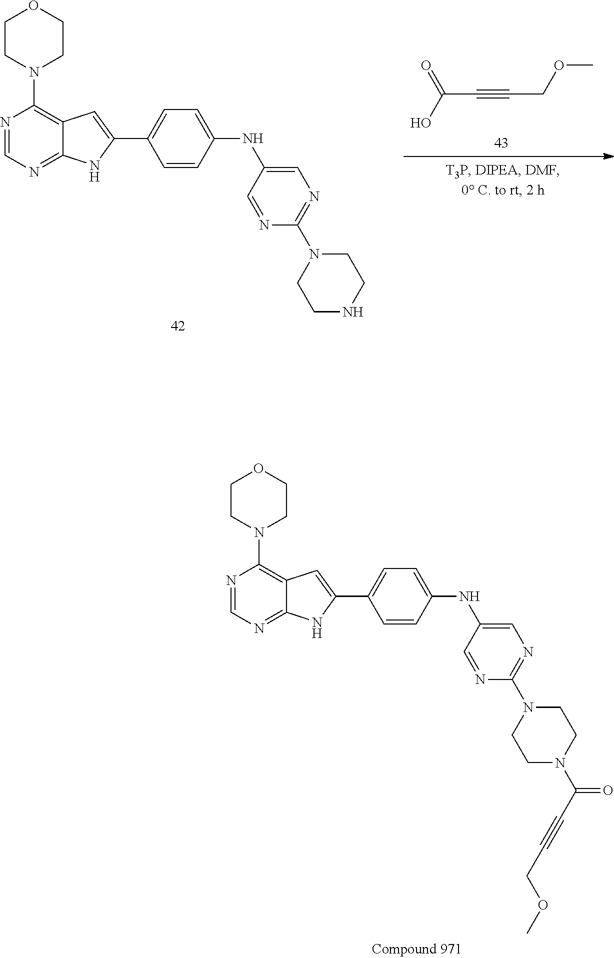

### Synthesis of 4-methoxy-1-(4-(5-((4-(4-morpholino-7H-pyrrolo[2,3-d]pyrimidin-6-yl)phenyl)amino)pyrimidin-2-yl)piperazin-1-yl)but-2-yn-1-one (Compound 971)

To a stirred solution of N-(4-(4-morpholino-7H-pyrrolo[2,3-d]pyrimidin-6-yl)phenyl)-2-(piperazin-1-yl)pyrimidin-5-amine 42 (250 mg, 0.546 mmol) in DMF (5 mL) were added DIPEA (0.3 mL, 1.64 mmol), 4-methoxybut-2-ynoic acid 43 (75 mg, 0.656 mmol) and T3P (0.6 mL, 0.52 mmol) sequentially at 0° C. under N₂ atmosphere. The reaction mixture was stirred at RT for 2 h. After completion of reaction (monitored by TLC), the reaction mixture was poured into ice cold water (50 mL) and extracted with EtOAc (3×50 mL). The combined organic layer was washed with ice cold brine (2×50 mL), dried over Na₂SO₄, filtered and concentrated under reduced pressure. The crude was purified by flash column chromatography (silica gel 100-200 M, 12 g SNAP) using eluents 3-5% MeOH in DCM to get title Compound 971 (77 mg, 25%).

¹H NMR (400 MHz, DMSO-d₆): δ 12.02 (brs, 1H), 8.34 (s, 2H), 8.13 (s, 1H), 7.97 (s, 1H), 7.70 (d, J=8.4 Hz, 2H), 6.92 (d, J=1.2 Hz, 1H), 6.83 (d, J=8.8 Hz, 2H), 4.33 (s, 2H), 3.86-3.83 (m, 4H), 3.78-3.73 (m, 4H), 3.74-3.71 (m, 6H), 3.61-3.58 (m, 2H), 3.32 (s, 3H).

LCMS=[M+H]⁺: 554.37; Purity=98.43%.

### Example 116

### Compound 972

Preparation of(S)-4-hydroxy-1-(4-(2,2,2-trifluoro-1-((4-(4-morpholino-1H-pyrrolo[3,2-c]pyridin-2-yl)phenyl)amino)ethyl)piperidin-1-yl)but-2-yn-1-one (Compound 972)

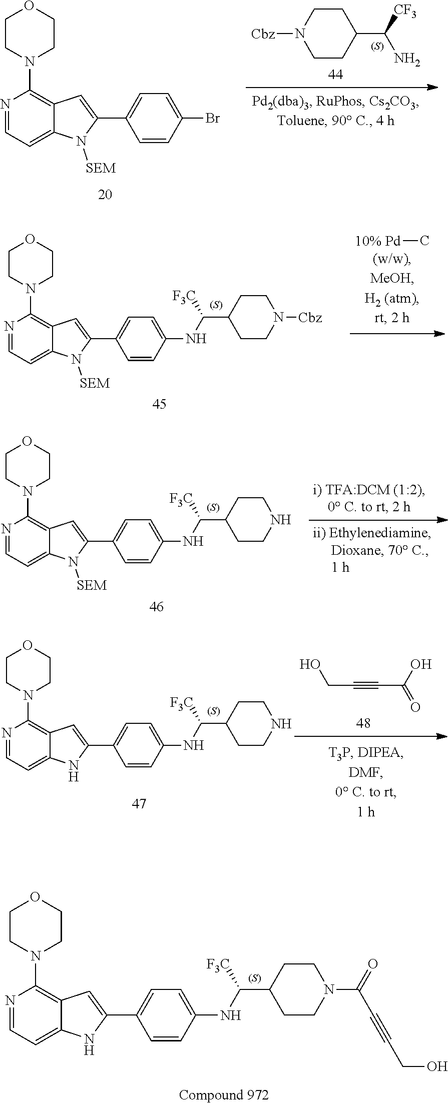

### Synthesis of benzyl(S)-4-(2,2,2-trifluoro-1-((4-(4-morpholino-1-((2-(trimethylsilyl)ethoxy)methyl)-1H-pyrrolo[3,2-c]pyridin-2-yl)phenyl)amino)ethyl)piperidine-1-carboxylate (45)

To a stirred solution of 4-(2-(4-bromophenyl)-1-((2-(trimethylsilyl)ethoxy)methyl)-1H-pyrrolo[3,2-c]pyridin-4-yl)morpholine 20 (1.6 g, 3.28 mmol) and benzyl(S)-4-(1-amino-2,2,2-trifluoroethyl)piperidine-1-carboxylate 44 (1.55 g, 4.91 mmol) in toluene (15 mL) was added Cs₂CO₃ (2.2 g, 6.55 mmol) at RT. The reaction mixture was degassed with N₂ gas for 10 min, followed by RuPhos (306 mg, 0.655 mmol) and Pd₂(dba)₃ (300 mg, 0.328 mmol). The reaction mixture was stirred at 90° C. for 4 h. After completion of reaction (monitored by TLC and LCMS), the reaction mixture was filtered through celite pad and washed with EtOAc (100 mL). The filtrate was dilute with water (60 mL) and extracted with EtOAc (3×80 mL). The combined organic layer was dried over Na₂SO₄, filtered and concentrated under reduced pressure. The crude was purified by flash column chromatography (silica gel 100-200 M, 24 g SNAP) using eluents 30-35% EtOAc in heptane to get the title compound as a light brown solid 45 (2 g, 70%).

LCMS=[M+H]⁺: 724.45; Purity=80%.

### Synthesis of (S)-4-(4-morpholino-1-((2-(trimethylsilyl)ethoxy)methyl)-1H-pyrrolo[3,2-c]pyridin-2-yl)-N-(2,2,2-trifluoro-1-(piperidin-4-yl)ethyl)aniline (46)

To a solution of benzyl(S)-4-(2,2,2-trifluoro-1-((4-(4-morpholino-1-((2-stirred (trimethylsilyl)ethoxy)methyl)-1H-pyrrolo[3,2-c]pyridin-2-yl)phenyl)amino)ethyl)piperidine-1-carboxylate 45 (2 g, 2.76 mmol) in MeOH (30 mL) was added 10% Pd—C(2 g) at RT under H₂ atmosphere pressure and stirred at RT for 2 h. After completion of reaction (monitored by TLC and LCMS), the reaction mixture was filtered through celite pad and washed with MeOH (100 mL). The filtrate was concentrated under reduced pressure to get the title compound as a yellow solid 46 (1 g; crude), which was used in next step directly without further purification.

LCMS=[M+H]⁺: 590.41; Purity=75%.

### Synthesis of (S)-4-(4-morpholino-1H-pyrrolo[3,2-c]pyridin-2-yl)-N-(2,2,2-trifluoro-1-(piperidin-4-yl)ethyl)aniline (47)

To a stirred solution of(S)-4-(4-morpholino-1-((2-(trimethylsilyl)ethoxy)methyl)-1H-pyrrolo[3,2-c]pyridin-2-yl)-N-(2,2,2-trifluoro-1-(piperidin-4-yl)ethyl)aniline 46 (1 g, 1.69 mmol) in DCM (20 mL) was added TFA (10 mL) dropwise at 0° C. and stirred at RT for 2 h. After completion of reaction (monitored by TLC and LCMS), the reaction mixture was concentrated under reduced pressure, triturated with Et₂O and dried under reduced pressure. The obtained crude was dissolved in 1,4-dioxane (10 mL) and ethylenediamine (0.2 mL, 2.54 mmol) was added and stirred at 70° C. for 30 min. After completion of reaction (monitored by LCMS), the reaction was poured into ice cold water (50 mL) and extracted with 25% IPA in CHCl₃ (3×80 mL). The combined organic layer was dried over Na₂SO₄, filtered and concentrated under reduced pressure to get the title compound as a yellow solid 47 (600 mg; crude), which was used in next step directly without further purification.

LCMS=[M+H]⁺: 460.32; Purity=55%.

### Synthesis of (S)-4-hydroxy-1-(4-(2,2,2-trifluoro-1-((4-(4-morpholino-1H-pyrrolo[3,2-c]pyridin-2-yl)phenyl)amino)ethyl)piperidin-1-yl)but-2-yn-1-one (Compound 972)

To a stirred solution of(S)-4-(4-morpholino-1H-pyrrolo[3,2-c]pyridin-2-yl)-N-(2,2,2-trifluoro-1-(piperidin-4-yl)ethyl)aniline 47 (400 mg, 0.87 mmol) in DMF (8 mL) were added DIPEA (0.5 mL, 2.61 mmol), 4-hydroxybut-2-ynoic acid 48 (105 mg, 1.04 mmol) and T3P (1.0 mL, 1.13 mmol) sequentially at 0° C. under N₂ atmosphere. The reaction mixture was stirred at RT for 1 h. After completion of reaction (monitored by TLC), the reaction mixture was poured into ice cold water (50 mL) and extracted with EtOAc (3×50 mL). The combined organic layer was washed with ice cold brine (2×30 mL), dried over Na₂SO₄, filtered and concentrated under reduced pressure. The crude was purified by flash column chromatography (silica gel 100-200 M, 12 g SNAP) using eluents 5-8% MeOH in DCM to get title Compound 972 (18 mg, 3%).

¹H NMR (400 MHz, DMSO-d₆): δ 11.65 (brs, 1H), 7.67 (d, J=6 Hz, 1H), 7.63 (d, J=8.4 Hz, 2H), 6.90-6.88 (m, 4H), 6.15-6.12 (m, 1H), 5.47-5.41 (m, 1H), 4.41-4.27 (m, 3H), 4.23 (t, J=6.4 Hz, 2H), 3.79-3.77 (m, 4H), 3.66-3.63 (m, 4H), 3.26-3.10 (m, 1H), 2.74-2.67 (m, 1H), 2.16-2.13 (m, 1H), 1.90-1.74 (m, 2H), 1.49-1.35 (m, 2H).

LCMS=[M+H]⁺: 542.17; Purity=95.01%.

### Example 117

### Compound 973

### Preparation of (E)-N-(4-(4-morpholino-7H-pyrrolo[2,3-d]pyrimidin-6-yl)phenyl)-2-(3-(prop-1-en-1-ylsulfonyl)piperidin-1-yl)pyrimidin-5-amine (Compound 973)

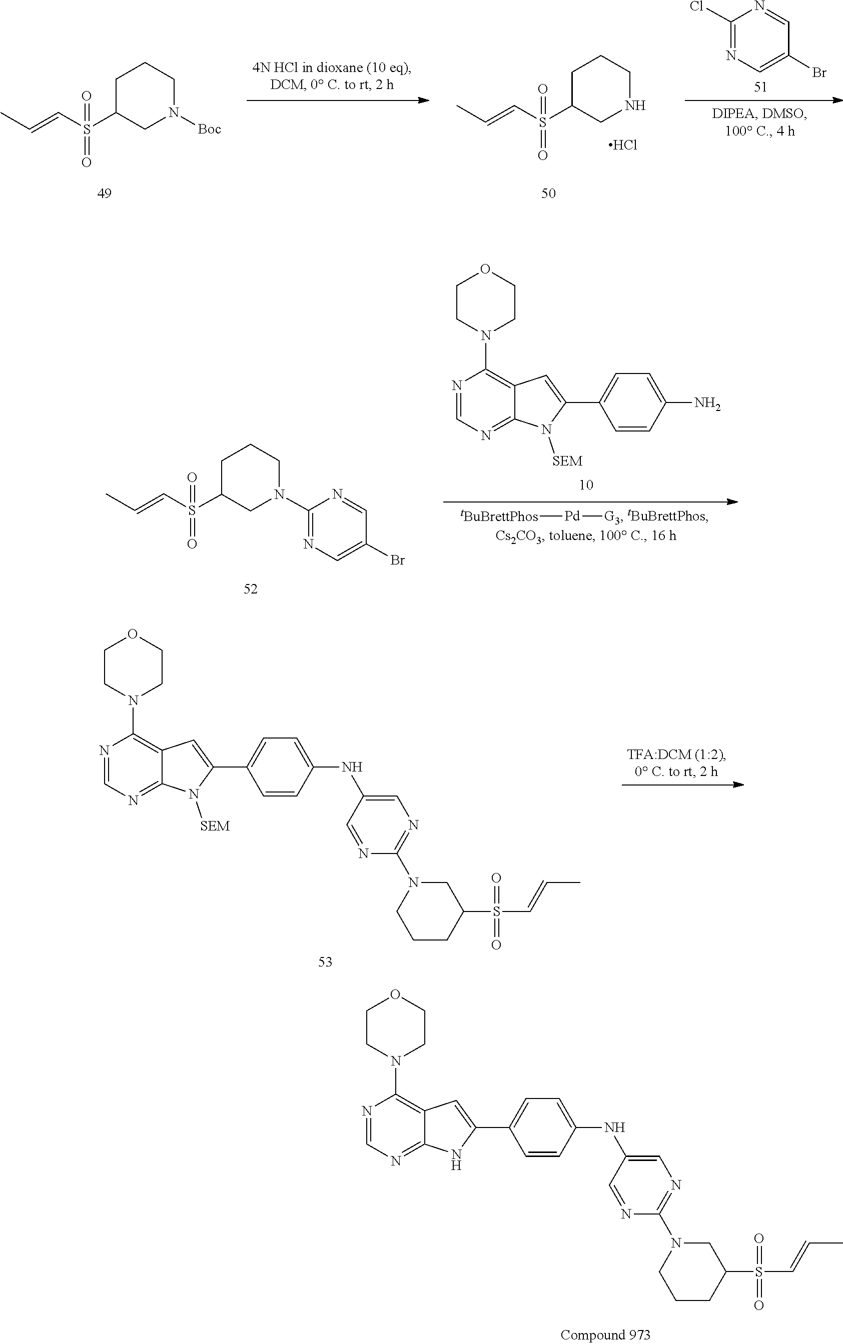

### Synthesis of (E)-3-(prop-1-en-1-ylsulfonyl)piperidine hydrochloride (50)

To a stirred solution of tert-butyl (E)-3-(prop-1-en-1-ylsulfonyl)piperidine-1-carboxylate 49 (400 mg, 1.38 mmol) in DCM (5 mL) was added 4N HCl in 1,4-Dioxane (2.5 mL) drop wise at 0° C. under N₂ atmosphere. The reaction mixture was stirred at RT for 2 h. After completion of reaction (monitored by TLC), the reaction mixture was concentrated under reduced pressure, triturated with Et₂O and dried over reduced pressure to get the title compound as a colorless semi solid 50 (250 mg, crude as HCl salt), which was used in next step directly without further purification.

LCMS=[M+H]⁺: 190.11; Purity=99%.

### Synthesis of (E)-5-bromo-2-(3-(prop-1-en-1-ylsulfonyl)piperidin-1-yl)pyrimidine (52)

To a stirred solution of (E)-3-(prop-1-en-1-ylsulfonyl)piperidine hydrochloride 50 (250 mg, 1.32 mmol) in DMSO (5 mL) was added DIPEA (1.2 mL, 6.60 mmol) at RT and stirred for 5 min. Then 5-bromo-2-chloropyrimidine 51 (380 mg, 1.98 mmol) was added and stirred at 100° C. for 4 h. After completion of reaction (monitored by TLC), the reaction mixture was cooled to RT, quenched with ice cold-water (50 mL) and extracted with EtOAc (3×50 mL). The combined organic layer was washed with brine (2×30 mL), dried over Na2SO₄, filtered and evaporated under reduced pressure. The crude was purified by flash column chromatography (silica gel 100-200 M, 12 g SNAP) using eluents 20-30% EtOAc in heptane to get the title compound as a yellow solid 52 (230 mg, 50%).

LCMS=[M+H]⁺: 346.05; Purity=90%.

### Synthesis of (E)-5-bromo-2-(3-(prop-1-en-1-ylsulfonyl)piperidin-1-yl)pyrimidine (53)

To a stirred solution of (E)-5-bromo-2-(3-(prop-1-en-1-ylsulfonyl)piperidin-1-yl)pyrimidine 52 (150 mg, 0.433 mmol) and 4-(4-morpholino-7-((2-(trimethylsilyl)ethoxy)methyl)-7H-pyrrolo[2,3-d]pyrimidin-6-yl)aniline 10 (221 mg, 0.52 mmol) in toluene (5 mL) was added Cs₂CO₃ (282 mg, 0.866 mmol) at RT. The reaction mixture was purged with N₂ gas for 10 min, followed by ^{t}BuBrettPhos-Pd-G3 (39.3 mg, 0.043 mmol) and ^{t}BuBrettPhos (46.5 mg, 0.086 mmol) was added and stirred at 100° C. for 16 h. After completion of reaction (monitored by TLC and LCMS), the reaction mixture was diluted with water (20 mL) and extracted with EtOAc (3×30 mL). The combined organic layer was dried over Na₂SO₄, filtered and evaporated under reduced pressure. The crude was purified by flash chromatography (silica gel 100-200 M, 12 g SNAP) using eluents 30-35% EtOAc in heptane to get the title compound as a brown solid 53 (200 mg, 67%).

LCMS=[M+H]⁺: 691.36; Purity=78%.

### Synthesis of (E)-N-(4-(4-morpholino-7H-pyrrolo[2,3-d]pyrimidin-6-yl)phenyl)-2-(3-(prop-1-en-1-ylsulfonyl)piperidin-1-yl)pyrimidin-5-amine (Compound 973)

To a stirred solution of (E)-N-(4-(4-morpholino-7-((2-(trimethylsilyl)ethoxy)methyl)-7H-pyrrolo[2,3-d]pyrimidin-6-yl)phenyl)-2-(3-(prop-1-en-1-ylsulfonyl)piperidin-1-yl)pyrimidin-5-amine 53 (160 mg, 0.289 mmol) in DCM (3 mL) was added TFA (1.5 mL) dropwise at 0° C. under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The crude was diluted with water (20 mL) and extracted with 25% IPA in CHCl₃ (3×40 mL). The combined organic layer was dried over Na₂SO₄, filtered and concentrated under reduced pressure. The crude was purified by RP-HPLC [2-98% 0.1% TFA in Water/ACN; Column: X Select Fluoro Phenyl C18 (19*250) 5μ] to get the title Compound 973 (22 mg, 17%).

¹H NMR (400 MHz, DMSO-d₆): δ 12.98 (brs, 1H), 8.35-8.34 (m, 2H), 8.27 (s, 1H), 8.02 (brs, 1H), 7.73 (d, J=8.4 Hz, 2H), 7.12 (brs, 1H), 6.85 (d, J=8.4 Hz, 2H), 6.68-6.64 (m, 1H), 5.54-5.44 (m, 1H), 5.00-4.90 (m, 1H), 4.56-4.50 (m, 1H), 4.00-3.98 (m, 1H), 3.92-3.90 (m, 4H), 3.80-3.78 (m, 4H), 3.23-3.11 (m, 3H), 2.93-2.87 (m, 1H), 2.22-2.09 (m, 1H), 1.96-1.94 (m, 2H), 1.83-1.69 (m, 2H), 1.53-1.44 (m, 1H).

LCMS=[M+H]⁺: 561.31; Purity=98%.

### Example 118

### Compound 1002

### Preparation of (R)-4-(dimethylamino)-N-methyl-N-(1-(5-((4-(4-morpholino-7H-pyrrolo[2,3-d]pyrimidin-6-yl)phenyl)amino)pyrimidin-2-yl)piperidin-3-yl)but-2-ynamide (Compound 1002)

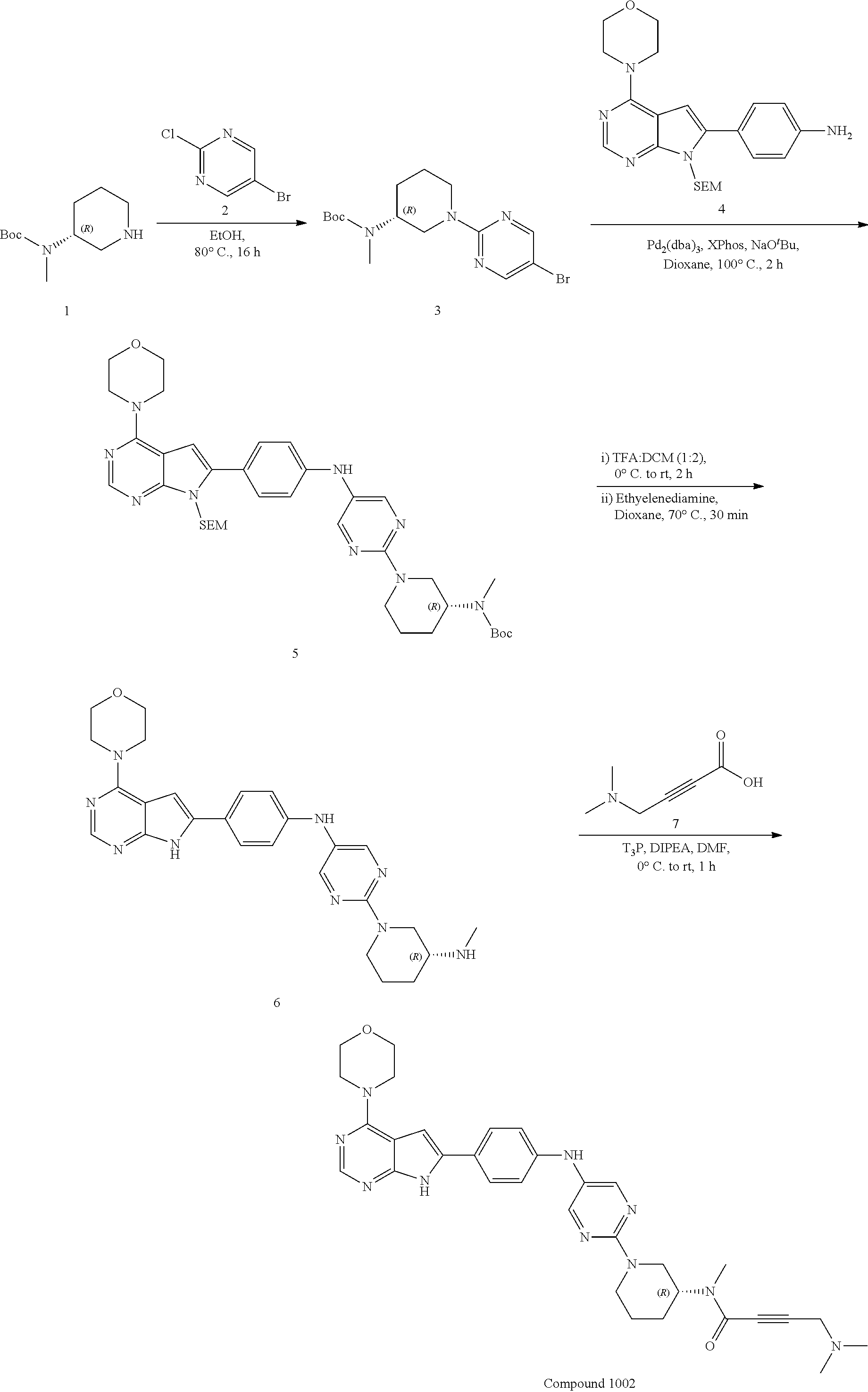

### Synthesis of tert-butyl (R)-(1-(5-bromopyrimidin-2-yl)piperidin-3-yl)(methyl)carbamate (3)

To a stirred solution of tert-butyl (R)-methyl (piperidin-3-yl)carbamate 1 (2 g, 9.33 mmol) in EtOH (20 mL) was added 5-bromo-2-chloropyrimidine 2 (1.81 g, 9.33 mmol) at RT under N₂ atmosphere and stirred the reaction mixture at 80° C. for 16 h. After completion of reaction (monitored by TLC), the reaction mixture was concentrated under reduced pressure. The crude was purified by flash column chromatography (silica gel 100-200 M, 40 g SNAP) using eluents 10-15% EtOAc in heptane to get the title compound 3 as white solid (1.7 g, 50%).

LCMS=[M+H]⁺: 371.28; Purity=99%.

### Synthesis of tert-butyl (R)-methyl (1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl)carbamate (5)

To a stirred solution of tert-butyl (R)-(1-(5-bromopyrimidin-2-yl)piperidin-3-yl)(methyl)carbamate 3 (1.6 g, 4.31 mmol) and 4-(4-morpholino-7-((2-(trimethylsilyl)ethoxy)methyl)-7H-pyrrolo[2,3-d]pyrimidin-6-yl)aniline 4 (1.83 g, 4.31 mmol) in 1,4-dioxane (20 mL) was added NaO^{t}Bu (621 mg, 6.46 mmol) at RT. The resulting reaction mixture was degassed with N₂ gas for 10 min, followed by added XPhos (411 mg, 0.862 mmol) and Pd₂(dba)₃ (395 mg, 0.431 mmol) and stirred at 100° C. for 2 h. After completion of reaction (monitored by TLC and LCMS), the reaction mixture was diluted with water (80 mL) and extracted with EtOAc (3×80 mL). The combined organic layer was dried over Na₂SO₄, filtered and evaporated under reduced pressure. The crude was purified by flash column chromatography (silica gel 100-200 M, 40 g SNAP) using eluents 30-40% EtOAc in heptane to get the title compound as a yellow solid 5 (2 g, 60%).

LCMS=[M+H]⁺: 716; Purity=90%

### Synthesis of (R)-2-(3-(methylamino)piperidin-1-yl)-N-(4-(4-morpholino-7H-pyrrolo[2,3-d]pyrimidin-6-yl)phenyl)pyrimidin-5-amine (6)

To an ice cold stirred solution of tert-butyl (R)-methyl (1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl)carbamate 5 (1 g, 1.40 mmol) in DCM (20 mL) was added TFA (10 mL) dropwise under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 1,4-dioxane (10 mL) and ethylenediamine (0.28 mL, 4.19 mmol) was added and stirred at 70° C. for 30 min. After completion of reaction (monitored by LCMS), the reaction was poured into ice cold water (50 mL) and extracted with 25% IPA in CHCl₃ (3×80 mL). The combined organic layer was dried over Na₂SO₄, filtered and concentrated under reduced pressure to get the title compound as a yellow solid 6 (500 mg; crude), which was used in next step directly without further purification.

LCMS=[M+H]⁺: 486.33; Purity=65%.

### Synthesis of (R)-4-(dimethylamino)-N-methyl-N-(1-(5-((4-(4-morpholino-7H-pyrrolo[2,3-d]pyrimidin-6-yl)phenyl)amino)pyrimidin-2-yl)piperidin-3-yl)but-2-ynamide (Compound 1002)

To a stirred solution of (R)-2-(3-(methylamino)piperidin-1-yl)-N-(4-(4-morpholino-7H-pyrrolo[2,3-d]pyrimidin-6-yl)phenyl)pyrimidin-5-amine 6 (500 mg, 1.03 mmol) in DMF (10 mL) was added DIPEA (0.5 mL, 3.09 mmol) at 0° C. under N₂ atmosphere. Then, 4-(dimethylamino)but-2-ynoic acid 7 (157 mg, 1.24 mmol) and T3P (1 mL, 1.14 mmol) were added sequentially to the reaction mixture and stirred at RT for 1 h. After completion of reaction (monitored by TLC and LCMS), the reaction mixture was poured into ice cold water (50 mL) and extracted with EtOAc (3×70 mL). The combined organic layer was washed with ice cold brine (2×50 mL), dried over Na₂SO₄, filtered and concentrated under reduced pressure. The crude was purified by flash column chromatography (silica gel 230-400 M, 12 g SNAP) using eluents 5-6% MeOH in DCM to get Compound 1002 (250 mg, 40%).

¹H NMR (400 MHz, DMSO-d₆): δ 12.01 (s, 1H), 8.31-8.30 (m, 2H), 8.14 (s, 1H), 7.93-7.92 (m, 1H), 7.72-7.69 (m, 2H), 6.92 (s, 1H), 6.81 (t, J=8.4 Hz, 2H), 4.60-4.51 (m, 2H), 4.35-4.17 (m, 1H), 3.86-3.83 (m, 4H), 3.74-3.72 (m, 4H), 3.48-3.38 (m, 2H), 3.15-2.98 (m, 1H), 2.85 (s, 3H), 2.82-2.75 (m, 1H), 2.22 (s, 3H), 2.04 (s, 3H), 1.91-1.82 (m, 3H), 1.50-1.44 (m, 1H).

LCMS=[M+H]⁺: 595.24; Purity=96.06%.

### Example 119

### Compound 1006 and Compound 967

### Preparation of 1-(4-((R)-3-((R)-2,2,2-trifluoro-1-((4-(4-morpholino-7H-pyrrolo[2,3-d]pyrimidin-6-yl)phenyl)amino)ethyl)pyrrolidin-1-yl)piperidin-1-yl)prop-2-en-1-one (Compound 1006) and 1-(4-((R)-3-((S)-2,2,2-trifluoro-1-((4-(4-morpholino-7H-pyrrolo[2,3-d]pyrimidin-6-yl)phenyl)amino)ethyl)pyrrolidin-1-yl)piperidin-1-yl)prop-2-en-1-one (Compound 967)

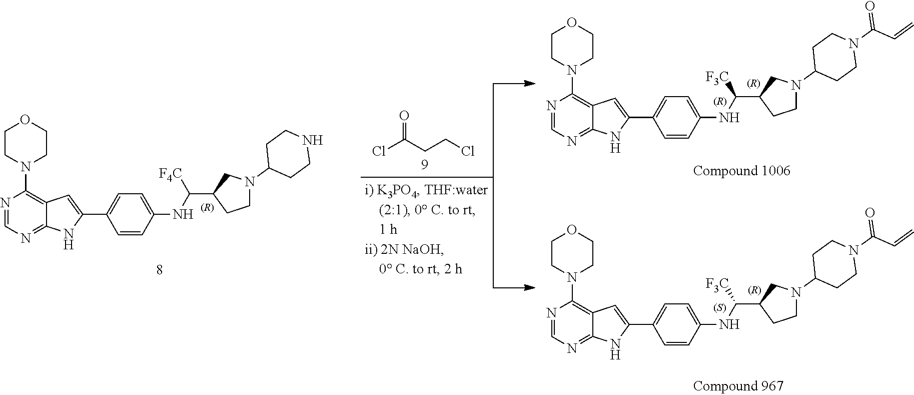

To an ice cold stirred solution of 4-(4-morpholino-7H-pyrrolo[2,3-d]pyrimidin-6-yl)-N-(2,2,2-trifluoro-1-((R)-1-(piperidin-4-yl)pyrrolidin-3-yl)ethyl)aniline 8 (742 mg, 1.4 mmol) in THF (10 mL) was added K₃PO₄ (595 mg, 2.8 mmol) dissolved in water (5 mL) and stirred for 5 min. Then a solution of 3-chloropropanoyl chloride 9 (213 mg, 1.68 mmol) in THF (1 mL) was added dropwise at 0° C. and stirred at RT for 1 h. After completion of starting material (acylation reaction, monitored by TLC), 2N aq. NaOH (8.5 mL, 16.8 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [2-98% 0.1% TFA in water/ACN; Column: X Select CSH C18 (19*250) 10μ] to get Compound 1006 (20 mg, 2.5%) and Compound 967 (14 mg, 2%) as a separable diastereomers. [Note: Chirality arbitrarily assigned]

Analytical data for Compound 1006: 1H NMR (400 MHz, DMSO-d₆): δ 12.24 (s, 1H), 8.20 (s, 1H), 7.72-7.69 (m, 2H), 6.99 (brs, 1H), 6.88-6.79 (m, 3H), 6.43-6.35 (m, 1H), 6.11 (dd, J=16.8 Hz, 2.4 Hz, 1H), 5.69 (dd, J=10.4 Hz, 2.0 Hz, 1H), 4.67-4.63 (m, 1H), 4.52-4.49 (m, 1H), 4.18-4.15 (m, 1H), 3.88-3.86 (m, 4H), 3.77-3.75 (m, 4H), 3.71-3.68 (m, 2H), 3.57-3.55 (m, 1H), 3.44-3.38 (m, 1H), 3.28-3.12 (m, 2H), 3.02-2.97 (m, 1H), 2.62-2.56 (m, 1H), 2.20-2.07 (m, 4H), 1.42 (brs, 2H).

LCMS=[M+H]⁺: 584.32; Purity=96.63%.

Analytical data for Compound 967: 1H NMR (400 MHz, DMSO-d₆): δ 11.97 (s, 1H), 8.13 (s, 1H), 7.64 (d, J=8.4 Hz, 2H), 6.88 (brs, 1H), 6.81 (d, J=8.8 Hz, 2H), 6.78-6.71 (m, 1H), 6.19 (d, J=9.6 Hz, 1H), 6.07-6.02 (m, 1H), 5.62 (t, J=8.8 Hz, 1H), 4.35-4.29 (m, 1H), 4.19-4.16 (m, 1H), 3.99-3.90 (m, 1H), 3.85-3.83 (m, 4H), 3.74-3.72 (m, 4H), 3.08-3.05 (m, 1H), 2.87-2.67 (m, 4H), 2.36-2.33 (m, 1H), 2.27-2.20 (m, 1H), 1.96-1.90 (m, 1H), 1.79-1.77 (m, 2H), 1.70-1.68 (m, 1H), 1.25-1.23 (m, 2H). [1H may merged with solvent peak]LCMS=[M+H]⁺: 584.32; Purity=99.55%.

### Example 120

### Compound 1010 and Compound 948

### Preparation of 1-(4-((S)-3-((R)-2,2,2-trifluoro-1-((4-(4-morpholino-7H-pyrrolo[2,3-d]pyrimidin-6-yl)phenyl)amino)ethyl)pyrrolidin-1-yl)piperidin-1-yl)prop-2-en-1-one (Compound 1010) and 1-(4-((S)-3-((S)-2,2,2-trifluoro-1-((4-(4-morpholino-7H-pyrrolo[2,3-d]pyrimidin-6-yl)phenyl)amino)ethyl)pyrrolidin-1-yl)piperidin-1-yl)prop-2-en-1-one (Compound 948)

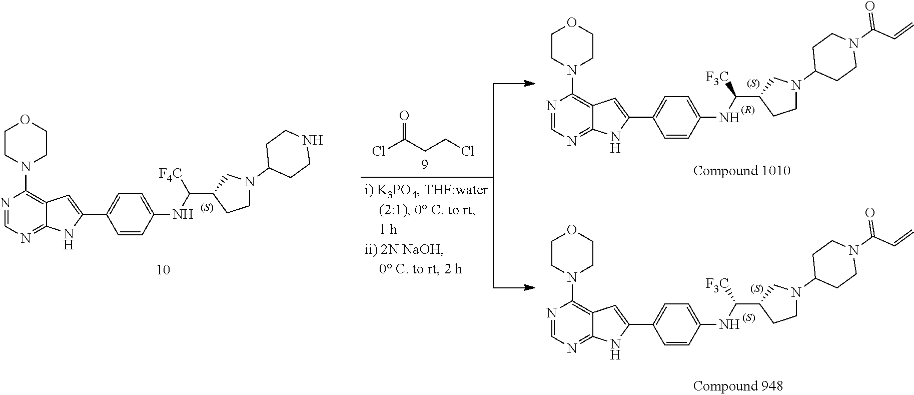

To a stirred solution of 4-(4-morpholino-7H-pyrrolo[2,3-d]pyrimidin-6-yl)-N-(2,2,2-trifluoro-1-((S)-1-(piperidin-4-yl)pyrrolidin-3-yl)ethyl)aniline 10 (616 mg, 1.17 mmol) in THF (10 mL) was added K₃PO₄ (495 mg, 2.33 mmol) dissolved in water (5 mL) at 0° C. and stirred for 5 min. Then a solution of 3-chloropropanoyl chloride 9 (178 mg, 1.4 mmol) in THF (1.5 mL) was added dropwise and stirred at RT for 1 h. After completion of starting material (acylation reaction, monitored by TLC), 2N aq. NaOH (7 mL, 14 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [5-95% 5 mM Ammonium Bicarbonate in Water/ACN; Column: X Select CSH C18 (19*250) 10μ] to get Compound 1010 (33 mg, 4.5%) and Compound 948 (53 mg, 7%) as a separable diastereomers. [Note: Chirality arbitrarily assigned]

Analytical data for Compound 1010: ¹H NMR (400 MHz, DMSO-d₆): δ 11.97 (s, 1H), 8.13 (s, 1H), 7.64 (d, J=8.4 Hz, 2H), 6.88 (d, J=1.7 Hz, 1H), 6.83 (d, J=8.8 Hz, 2H), 6.77-6.73 (m, 1H), 6.15 (d, J=9.6 Hz, 1H), 6.05 (dd, J=16.4 Hz, 2.4 Hz, 1H), 5.64-5.61 (m, 1H), 4.36-4.30 (m, 1H), 4.19-4.16 (m, 1H), 3.96-3.89 (m, 1H), 3.85-3.83 (m, 4H), 3.78-3.73 (m, 4H), 3.14-3.04 (m, 1H), 2.89-2.82 (m, 2H), 2.72-2.66 (m, 1H), 2.41-2.36 (m, 2H), 2.32-2.23 (m, 2H), 1.97-1.90 (m, 1H), 1.87-1.78 (m, 2H), 1.69-1.59 (m, 1H), 1.25-1.17 (m, 2H).

LCMS=[M+H]⁺: 584.32; Purity=93.53%.

Analytical data for Compound 948: 1H NMR (400 MHz, DMSO-d₆): δ 11.96 (s, 1H), 8.14 (s, 1H), 7.64 (d, J=8.4 Hz, 2H), 6.88 (d, J=1.6 Hz, 1H), 6.81 (d, J=8.4 Hz, 2H), 6.78-6.72 (m, 1H), 6.19 (d, J=9.6 Hz, 1H), 6.07-6.02 (m, 1H), 5.62 (t, J=8.8 Hz, 1H), 4.33-4.31 (m, 1H), 4.19-4.16 (m, 1H), 3.95-3.90 (m, 1H), 3.85-3.80 (m, 4H), 3.75-3.72 (m, 4H), 3.10-3.07 (m, 1H), 2.88-2.76 (m, 3H), 2.38-2.30 (m, 3H), 2.22-2.20 (m, 1H), 1.92-1.89 (m, 1H), 1.80-1.77 (m, 2H), 1.70-1.67 (m, 1H), 1.23-1.21 (m, 2H).

LCMS=[M+H]⁺: 584.29; Purity=88.11%.

### Example 121

### Compound 963 and Compound 1018

### Preparation of 1-((3S,3′R)-3-(2,2,2-trifluoro-1-((4-(4-morpholino-7H-pyrrolo[2,3-d]pyrimidin-6-yl)phenyl)amino)ethyl)-[1,3′-bipyrrolidin]-1′-yl)prop-2-en-1-one (Compound 963) and 1-((3S,3'S)-3-(2,2,2-trifluoro-1-((4-(4-morpholino-7H-pyrrolo[2,3-d]pyrimidin-6-yl)phenyl)amino)ethyl)-[1,3′-bipyrrolidin]-1′-yl)prop-2-en-1-one (Compound 1018)

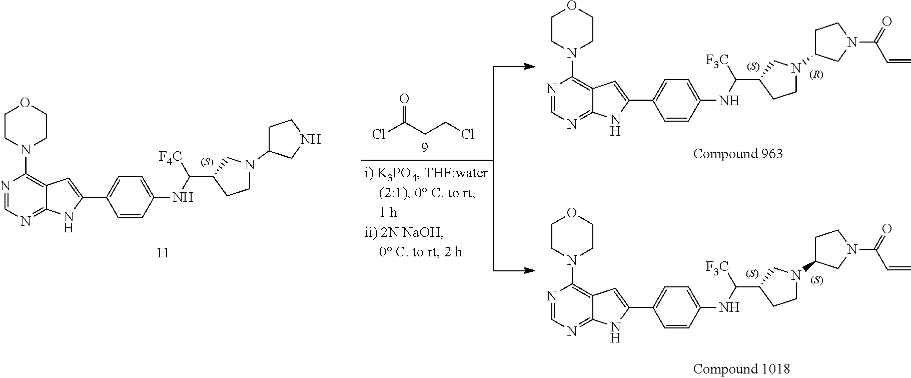

To an ice cold stirred solution of N-(1-((3S)-[1,3′-bipyrrolidin]-3-yl)-2,2,2-trifluoroethyl)-4-(4-morpholino-7H-pyrrolo[2,3-d]pyrimidin-6-yl)aniline 11 (500 mg, 0.970 mmol) in THF (6 mL) was added K₃PO₄ (412 mg, 1.94 mmol) dissolved in water (3 mL) under N₂ atmosphere and stirred for 5 min. Then a solution of 3-chloropropionyl chloride 9 (140 μL, 1.45 mmol) in THF (1 mL) was added drop wise to the reaction mixture at 0° C. and stirred at RT for 1 h. After completion of starting material (acylation reaction, monitored by TLC), 2N aq. NaOH (5.5 mL, 11.6 mmol) was added to the reaction mixture at 0° C. and stirred at RT for 2 h. After completion of reaction (complete elimination, monitored by TLC and LCMS), the reaction mixture was diluted with water (40 mL) and extracted with 2-MeTHF (3×50 mL). The combined organic layer was dried over Na₂SO₄, filtered and concentrated under reduced pressure. The crude was purified by RP-HPLC [5-95% 5 mM Ammonium bicarbonate in water/ACN; Column: X Select Phenyl Hexyl (19*250) 5μ] to get Compound 963 (37 mg, 7%) and Compound 1018 (7 mg, 1.3%) as a separable diastereomers. [Note: Chirality arbitrarily assigned].

Analytical data for Compound 963: 1H NMR (400 MHz, DMSO-d₆): δ11.97 (s, 1H), 8.13 (s, 1H), 7.69-7.67 (m, 2H), 6.90-6.87 (m, 4H), 6.59-6.42 (m, 2H), 6.17-6.13 (m, 1H), 5.69 (brs, 1H), 4.70-4.65 (m, 1H), 4.34-4.25 (m, 2H), 3.99-3.83 (m, 4H), 3.75-3.69 (m, 5H), 3.58-3.54 (m, 4H), 3.12-2.90 (m, 1H), 2.81-2.62 (m, 1H), 2.32-2.24 (m, 2H), 2.12-1.77 (m, 2H).

LCMS=[M+H]⁺: 570.28; Purity=95.06%.

Analytical data for Compound 1018: 1H NMR (400 MHz, DMSO-d₆): δ 11.97 (s, 1H), 8.13 (s, 1H), 7.65 (d, J=8.4 Hz, 2H), 6.88 (s, 1H), 6.83 (d, J=8.4 Hz, 2H), 6.62-6.51 (m, 1H), 6.17 (d, J=10.0 Hz, 1H), 6.13-6.07 (m, 1H), 5.66-5.60 (m, 1H), 4.35-4.31 (m, 1H), 3.84-3.83 (m, 4H), 3.74-3.73 (m, 4H), 3.65-3.45 (m, 2H), 3.27-3.26 (m, 1H), 3.18-3.09 (m, 1H), 2.90-2.79 (m, 2H), 2.72-2.66 (m, 1H), 2.38-2.34 (m, 2H), 2.32-2.26 (m, 1H), 2.01-1.93 (m, 2H), 1.77-1.65 (m, 2H).

LCMS=[M+H]⁺: 570.30; Purity=95.09%.

### Example 122

### Compound 1019

### Preparation of (R)-2-(azetidin-1-ylmethyl)-N-(1-(5-((4-(4-morpholino-7H-pyrrolo[2,3-d]pyrimidin-6-yl)phenyl)amino)pyridin-2-yl)piperidin-3-yl)acrylamide (Compound 1019)

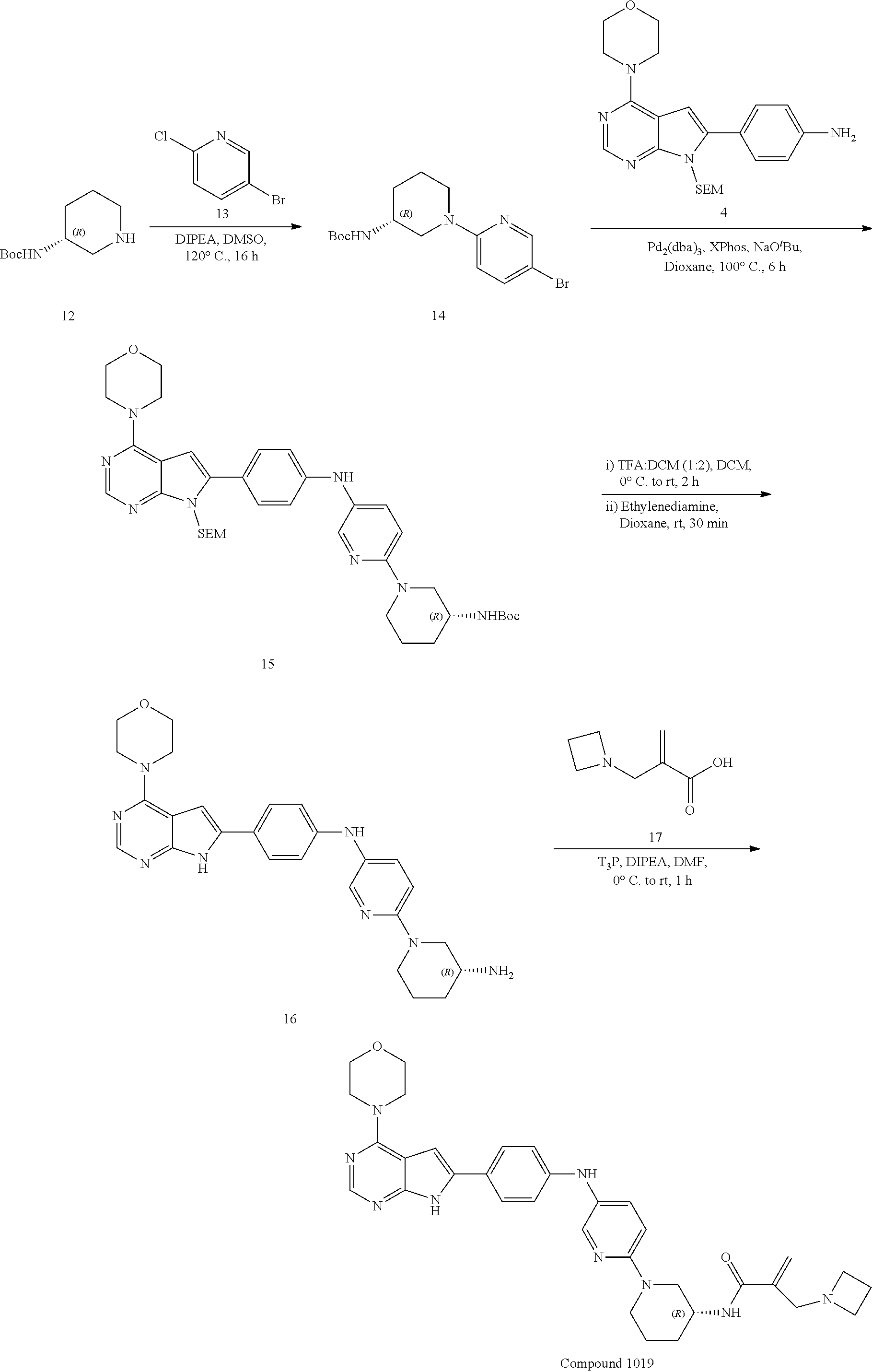

### Synthesis of tert-butyl (R)-(1-(5-bromopyridin-2-yl)piperidin-3-yl)carbamate (13)

To a stirred solution of tert-butyl (R)-piperidin-3-ylcarbamate 12 (2 g, 9.99 mmol) and 5-bromo-2-chloropyridine 13 (3.21 g, 12 mmol) in DMSO (20 mL) was added DIPEA (5.2 mL, 30 mmol) at RT. The resulting reaction mixture was stirred at 120° C. for 16 h. After completion of reaction (monitored by TLC and LCMS), the reaction mixture was poured into ice cold water (100 mL) and extracted with EtOAc (3×80 mL). The combined organic layer was washed with ice cold brine (2×50 mL), dried over Na₂SO₄ and concentrated under reduced pressure. The crude was purified by flash chromatography (silica gel 100-200 M, 24 g SNAP) using eluents 8-10% EtOAc in heptane to get the title as white solid compound 14 (1.8 g, 60%).

LCMS=[M+H]⁺: 358.11; Purity=97%.

### Synthesis of tert-butyl (R)-(1-(5-((4-(4-morpholino-7-((2-(trimethylsilyl)ethoxy)methyl)-7H-pyrrolo[2,3-d]pyrimidin-6-yl)phenyl)amino)pyridin-2-yl)piperidin-3-yl)carbamate (15)

To a stirred solution of tert-butyl (R)-(1-(5-bromopyridin-2-yl)piperidin-3-yl)carbamate 14 (1 g, 2.81 mmol) and 4-(4-morpholino-7-((2-(trimethylsilyl)ethoxy)methyl)-7H-pyrrolo[2,3-d]pyrimidin-6-yl)aniline 4 (1.19 g, 2.81 mmol) in 1,4-Dioxane (15 mL) was added NaO^{t}Bu (405 mg, 4.21 mmol) at RT. The resulting reaction mixture was degassed with N₂ gas for 10 min, followed by added XPhos (134 mg, 0.281 mmol) and Pd₂(dba)₃ (227 mg, 0.281 mmol) and stirred at 100° C. for 6 h. After completion of reaction (monitored by TLC and LCMS), the reaction mixture was diluted with water (80 mL) and extracted with EtOAc (3×80 mL). The combined organic layer was dried over Na₂SO₄, filtered and evaporated under reduced pressure. The crude was purified by flash column chromatography (silica gel 100-200 M, 40 g SNAP) using eluents 30-40% EtOAc in heptane to get the title compound as a yellow solid 15 (1.3 g, 67%).

LCMS=[M+H]⁺: 702.48; Purity=67%

### Synthesis of (R)-6-(3-aminopiperidin-1-yl)-N-(4-(4-morpholino-7H-pyrrolo[2,3-d]pyrimidin-6-yl)phenyl)pyridin-3-amine (16)

To an ice cold stirred solution of tert-butyl (R)-(1-(5-((4-(4-morpholino-7-((2-(trimethylsilyl)ethoxy)methyl)-7H-pyrrolo[2,3-d]pyrimidin-6-yl)phenyl)amino)pyridin-2-yl)piperidin-3-yl)carbamate 15 (1.2 g, 1.71 mmol) in DCM (20 mL) was added TFA (10 mL) dropwise under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 1,4-dioxane (10 mL) and ethylenediamine (0.170 mL, 2.57 mmol) was added and stirred at RT for 30 min. After completion of reaction (monitored by LCMS), the reaction was poured into ice cold water (60 mL) and extracted with 25% IPA in CHCl₃ (3×60 mL). The combined organic layer was dried over Na₂SO₄, filtered and concentrated under reduced pressure to get the title compound as a yellow solid 16 (600 mg; crude), which was used in next step directly without further purification.

LCMS=[M+H]⁺: 471.33; Purity=62%.

### Synthesis of (R)-2-(azetidin-1-ylmethyl)-N-(1-(5-((4-(4-morpholino-7H-pyrrolo[2,3-d]pyrimidin-6-yl)phenyl)amino)pyridin-2-yl)piperidin-3-yl)acrylamide (Compound 1019)

To an ice cold stirred solution of (R)-6-(3-aminopiperidin-1-yl)-N-(4-(4-morpholino-7H-pyrrolo[2,3-d]pyrimidin-6-yl)phenyl)pyridin-3-amine 16 (250 mg, 0.532 mmol) in DMF (5 mL) was added DIPEA (0.28 mL, 1.59 mmol). Then, 2-(azetidin-1-ylmethyl)acrylic acid 17 (0.15 mg, 1.06 mmol) and T3P (0.5 mL, 0.798 mmol) were added sequentially to the reaction mixture at 0° C. under N₂ atmosphere. The reaction mixture was stirred at RT for 1 h. After completion of reaction (monitored by TLC), the reaction mixture was poured into ice cold water (50 mL) and extracted with EtOAc (3×50 mL). The combined organic layer was washed with ice cold brine (2×30 mL), dried over Na₂SO₄, filtered and concentrated under reduced pressure. The crude residue was purified by RP-HPLC [5-95% 5 mM Ammonium Bicarbonate in Water/ACN; Column: X Bridge Sheild RP18 (19*250) 10μ] to get Compound 1019 (14 mg, 5%).

¹H NMR (400 MHz, DMSO-d₆): & 12.01 (s, 1H), 9.03 (d, J=7.6 Hz, 1H), 8.13 (s, 1H), 7.97 (d, J=2.4 Hz, 1H), 7.92 (s, 1H), 7.68 (d, J=8.4 Hz, 2H), 7.40 (dd, J=11.6 Hz, 2.4 Hz, 1H), 6.91 (s, 1H), 6.86-6.80 (m, 3H), 5.80 (s, 1H), 5.38 (s, 1H), 3.86-3.83 (m, 5H), 3.74-3.72 (m, 5H), 3.57-3.54 (m, 1H), 3.46-3.41 (m, 1H), 3.32-3.27 (m, 1H), 3.17-3.11 (m, 2H), 3.08-2.96 (m, 4H), 1.91-1.82 (m, 3H), 1.74-1.73 (m, 1H), 1.63-1.58 (m, 2H).

LCMS=[M+H]⁺: 594.34; Purity=95.58%.

### Example 123

### Compound 1033

### Preparation of 1-(4-(5-(hydroxy (4-(4-morpholino-7H-pyrrolo[2,3-d]pyrimidin-6-yl)phenyl)methyl)pyrimidin-2-yl)piperazin-1-yl)prop-2-en-1-one (Compound 1033)

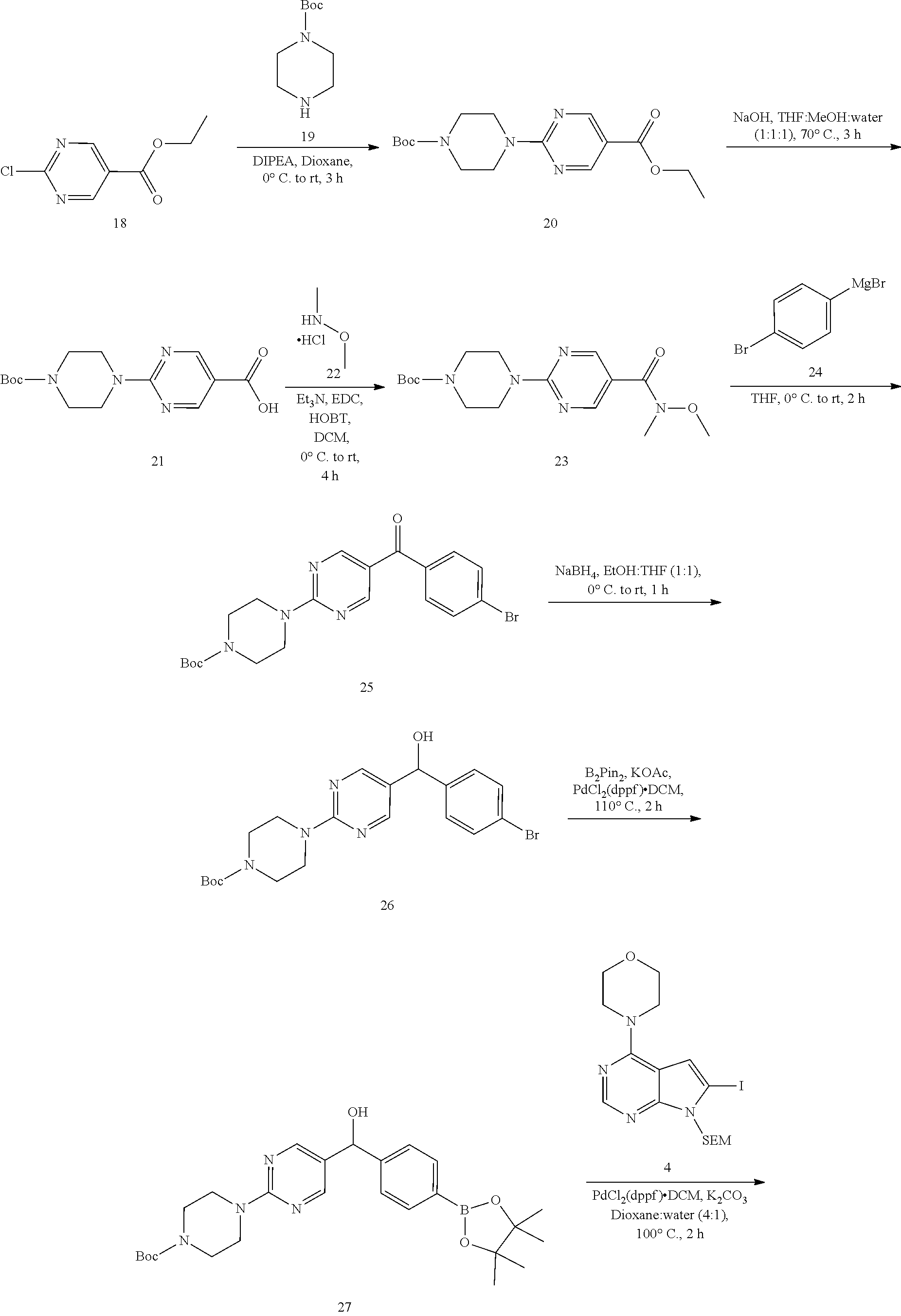

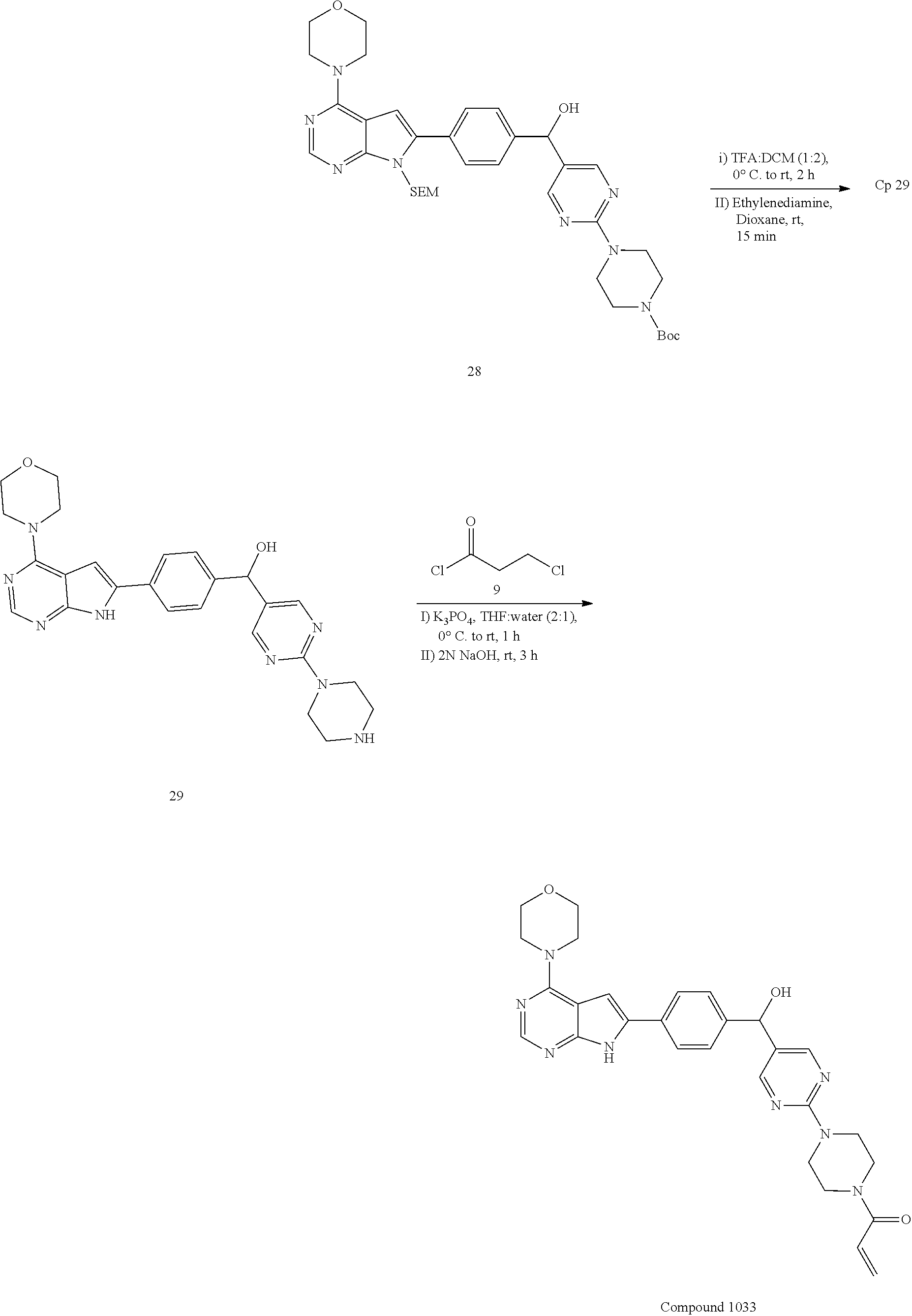 [SMILES: CC(C)(C)OC(=O)N1CCN(c2ncc(C(O)c3ccc(-c4cc5c(N6CCOCC6)ncnc5n4COCC[Si](C)(C)C)cc3)cn2)CC1 ; OC(c1ccc(-c2cc3c(N4CCOCC4)ncnc3[nH]2)cc1)c1cnc(N2CCNCC2)nc1 ; O=C(Cl)CCCl ; C=CC(=O)N1CCN(c2ncc(C(O)c3ccc(-c4cc5c(N6CCOCC6)ncnc5[nH]4)cc3)cn2)CC1]

### Synthesis of ethyl 2-(4-(tert-butoxycarbonyl) piperazin-1-yl)pyrimidine-5-carboxylate (20)

To an ice cold stirred solution of ethyl 2-chloropyrimidine-5-carboxylate 18 (1 g, 5.36 mmol) and tert-butyl piperazine-1-carboxylate 19 (998 mg, 5.36 mmol) in 1,4-dioxane (15 mL) was added DIPEA (2 mL, 11.8 mmol) at under N₂ atmosphere. The reaction mixture was stirred at RT for 3 h. After completion of reaction (TLC monitoring), the reaction mixture was concentrated under reduced pressure. The crude was purified by flash column chromatography (silica gel 230-400 M, 24 g SNAP) using eluents 25-30% EtOAc in heptane to get title compound as a white solid 20 (1.8 g, 98%).

LCMS=[M+H]⁺: 337.21; Purity=98.55%.

### Synthesis of 2-(4-(tert-butoxycarbonyl)piperazin-1-yl)pyrimidine-5-carboxylic acid (21)

To a stirred solution of ethyl 2-(4-(tert-butoxycarbonyl)piperazin-1-yl)pyrimidine-5-carboxylate 20 (1.8 g, 5.36 mmol) in THF (10 mL) and MeOH (10 mL) was added a aqueous solution of NaOH (429 mg, 10.7 mmol) in water (10 mL). The reaction mixture was stirred at 70° C. for 3 h. After completion of reaction (monitored by TLC), the reaction mixture was concentrated under reduced pressure. The crude was diluted with water (10 mL) and acidified with 1M HCl to pH~5. The obtained white solid precipitate was filtered and washed with ice cold water and dried under vacuum to get title compound as a white solid 21 (1.4 g; crude), which was used in next step directly without further purification.

LCMS=[M+H]⁺: 337.21; Purity=98.55%.

### Synthesis of tert-butyl 4-(5-(methoxy(methyl)carbamoyl)pyrimidin-2-yl)piperazine-1-carboxylate (23)

To a stirred solution of 2-(4-(tert-butoxycarbonyl)piperazin-1-yl)pyrimidine-5-carboxylic acid 21 (1.4 g, 4.54 mmol) in DCM (20 mL) were added Et₃N (2.53 mL, 18.2 mmol), HOBT (736 mg, 5.45 mmol) and EDC·HCl (1.31 g, 6.81 mmol) sequentially to the reaction mixture. The resulting reaction mixture was stirred under nitrogen atmosphere at room temperature for 1 h. Then, N,O-dimethylhydroxylamine hydrochloride 22 (531 mg, 5.45 mmol) was added to the reaction mixture. The resulting reaction mixture was stirred at RT for 4 h. After completion of reaction (monitored by TLC and LCMS), the reaction mixture was poured into water (50 mL) and extracted with DCM (3×60 mL). The combined organic layer was washed with ice cold brine solution (2×50 mL), dried over Na₂SO₄, filtered and evaporated under reduced pressure. The crude was purified by the flash chromatography (silica gel 230-400 M, 40 g SNAP) using eluents 30-40% EtOAc in heptane to get the title compound as a white solid 23 (1.5 g, 92%).

LCMS=[M+H]⁺: 352.17; Purity=98%.

### Synthesis of tert-butyl 4-(5-(4-bromobenzoyl)pyrimidin-2-yl)piperazine-1-carboxylate (25)

To an ice cold stirred solution of tert-butyl 4-(5-(methoxy(methyl)carbamoyl)pyrimidin-2-yl)piperazine-1-carboxylate 23 (500 mg, 1.42 mmol) and dry THF (10 mL) was added (4-bromophenyl) magnesium bromide 24 (22.8 mL, 11.4 mmol; 0.5 M in THF) dropwise to the reaction mixture under N₂ atmosphere. The reaction mixture was stirred at RT for 2 h. After completion of reaction (monitored by TLC and LCMS), the reaction mixture was quenched with 1N HCl (50 mL) and extracted with EtOAc (3×50 mL). The combined organic layer was dried over Na₂SO₄, filtered and concentrated under reduced pressure. The crude was purified by flash column chromatography (silica gel 230-400 M, 12 g SNAP) using eluents 10-12% EtOAc in heptane to get the title compound as a white solid 25 (500 mg, 51%).

LCMS=[M+H]⁺: 447.08; Purity=75%.

### Synthesis of tert-butyl 4-(5-((4-bromophenyl)(hydroxy)methyl)pyrimidin-2-yl)piperazine-1-carboxylate (26)

To an ice cold stirred solution of tert-butyl 4-(5-(4-bromobenzoyl)pyrimidin-2-yl)piperazine-1-carboxylate 25 (450 mg, 1.01 mmol) in EtOH (5 mL) and THF (5 mL) was added NaBH₄ (76.1 mg, 2.01 mmol) at same temperature under N₂ atmosphere. The reaction mixture was stirred at RT for 1 h. After completion of reaction (monitored by TLC), the reaction mixture was concentrated under reduced pressure. The crude residue was diluted with water (50 mL) and extracted with DCM (3×50 mL). The combined organic layer was washed with brine solution (2×30 mL), dried over Na₂SO₄, filtered and evaporated under reduced pressure. The crude was purified by flash column chromatography (silica gel 230-400M, 12 g SNAP) using eluents 25-30% EtOAc in heptane to get the title compound as white solid 26 (400 mg, 81%) LCMS=[M+H]⁺: 449.14; Purity=92%.

### Synthesis of tert-butyl 4-(5-(hydroxy (4-(4,4,5,5-tetramethyl-1,3,2-dioxaborolan-2-yl)phenyl)methyl)pyrimidin-2-yl)piperazine-1-carboxylate (27)

To a stirred solution of tert-butyl 4-(5-((4-bromophenyl)(hydroxy)methyl)pyrimidin-2-yl)piperazine-1-carboxylate 26 (350 mg, 0.779 mmol) and B₂Pin₂ (297 mg, 1.17 mmol) in 1,4-dioxane (10 mL) was added KOAc (229 mg, 2.34 mmol). The resulting reaction mixture was degassed with N₂ gas for 10 min, followed by added PdCl₂(dppf)·DCM (63.6 mg, 0.78 mmol) to the reaction mixture and stirred at 100° C. for 2 h. After completion of reaction (monitored by TLC and LCMS), the reaction mixture was filtered through celite bed and washed with EtOAc (100 mL). The filtrate was evaporated under reduced pressure to get the title compound as brown sticky oil 27 (490 mg; crude), which was used in next step directly without further purification.

### Synthesis of tert-butyl 4-(5-(hydroxy (4-(4-morpholino-7-((2-(trimethylsilyl)ethoxy)methyl)-7H-pyrrolo[2,3-d]pyrimidin-6-yl)phenyl)methyl)pyrimidin-2-yl)piperazine-1-carboxylate (28)

To a stirred solution of tert-butyl 4-(5-(hydroxy (4-(4,4,5,5-tetramethyl-1,3,2-dioxaborolan-2-yl)phenyl)methyl)pyrimidin-2-yl)piperazine-1-carboxylate 27 (485 mg, 0.978 mmol) and 4-(6-iodo-7-((2-(trimethylsilyl)ethoxy)methyl)-7H-pyrrolo[2,3-d]pyrimidin-4-yl)morpholine 4 (300 mg, 0.652 mmol) in 1,4-dioxane (10 mL) and water (2.5 mL) was added K₂CO₃ (270 mg, 1.95 mmol) at RT. The resulting reaction mixture was degassed with argon gas for 15 min. Then was added PdCl₂(dppf)·DCM (53 mg, 0.0652 mmol) and the resulting reaction mixture was stirred at 100° C. for 2 h. After the completion of reaction (monitored by TLC), the reaction mixture was diluted with water (50 mL) and extracted with EtOAc (3×80 mL). The combined organic layer was washed with brine solution (2×50 mL), dried over Na₂SO₄, filtered and evaporated under reduced pressure. The crude was purified by flash column chromatography (silica gel 230-400 M, 12 g SNAP) using eluents 50-60% EtOAc in heptane to get the title compound as a brown solid 28 (300 mg, 65%).

LCMS=[M+H]⁺: 703.45; Purity=93%.

### Synthesis of (4-(4-morpholino-7H-pyrrolo[2,3-d]pyrimidin-6-yl)phenyl)(2-(piperazin-1-yl)pyrimidin-5-yl)methanol (29)

To an ice cold stirred solution of tert-butyl 4-(5-(hydroxy (4-(4-morpholino-7-((2-(trimethylsilyl)ethoxy)methyl)-7H-pyrrolo[2,3-d]pyrimidin-6-yl)phenyl)methyl)pyrimidin-2-yl)piperazine-1-carboxylate 28 (300 mg, 0.427 mmol) in DCM (6 mL) was added TFA (3 mL) dropwise under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 1,4-dioxane (6 mL) and ethylenediamine (43 μL, 0.640 mmol) was added and stirred at RT for 15 min. After completion of reaction (monitored by LCMS), the reaction was poured into ice cold water (50 mL) and extracted with 25% IPA in CHCl₃ (3×40 mL). The combined organic layer was dried over Na₂SO₄, filtered and concentrated under reduced pressure to get the title compound as a pale yellow solid 29 (200 mg; crude), which was used in next step directly without further purification.

LCMS=[M+H]⁺: 473.24; Purity=93%.

### Synthesis of 1-(4-(5-(hydroxy (4-(4-morpholino-7H-pyrrolo[2,3-d]pyrimidin-6-yl)phenyl)methyl)pyrimidin-2-yl)piperazin-1-yl)prop-2-en-1-one (Compound 1033)

To an ice cold stirred solution of (4-(4-morpholino-7H-pyrrolo[2,3-d]pyrimidin-6-yl)phenyl)(2-(piperazin-1-yl)pyrimidin-5-yl)methanol 29 (210 mg, 0.444 mmol) in THF (6 mL) was added K₃PO₄ (189 mg, 0.889 mmol) dissolved in water (3 mL) at same temperature under N₂ atmosphere. Then a solution of 3-chloropropanoyl chloride 9 (51 μL, 0.533 mmol) in THF (1 mL) was added dropwise to the reaction mixture and stirred at RT for 1 h. After completion of starting material (acylation reaction, monitored by TLC), 2N aq. NaOH (2.7 mL, 5.33 mmol) was added to the reaction mixture and stirred at RT for 3 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by flash column chromatography (silica gel 230-400 M, 12 g SNAP) using eluents 5-7% MeOH in DCM to get Compound 1033 (105 mg, 42%).

¹H NMR (400 MHz, DMSO-d₆): δ 12.22 (s, 1H), 8.37 (s, 2H), 8.17 (s, 1H), 7.87 (d, J=8.0 Hz, 2H), 7.43 (d, J=8.0 Hz, 2H), 7.15 (s, 1H), 6.82 (dd, J=16.8 Hz, 10.4 Hz, 1H), 6.13 (dd, J=16.8 Hz, 2.0 Hz, 1H), 5.98 (d, J=4.0 Hz, 1H), 5.71-5.66 (m, 2H), 3.88-3.86 (m, 4H), 3.75-3.73 (m, 8H), 3.62-3.59 (m, 4H).

LCMS=[M+H]⁺: 527.23; Purity=93.84%.

### Example 124

### Compound 1036

### Preparation of 1-(4-(5-((4-(4-(3-oxa-8-azabicyclo[3.2.1]octan-8-yl)-7H-pyrrolo[2,3-d]pyrimidin-6-yl)phenyl)amino)pyrimidin-2-yl)piperazin-1-yl)-4-methoxybut-2-yn-1-one (Compound 1036)

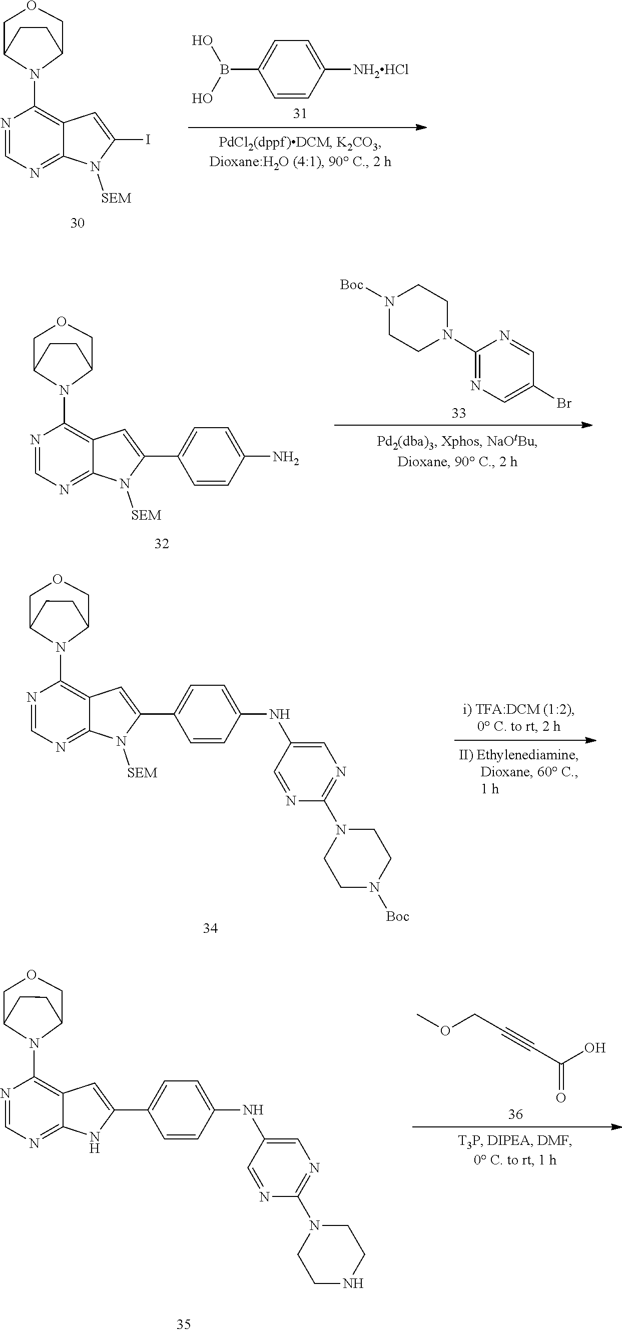

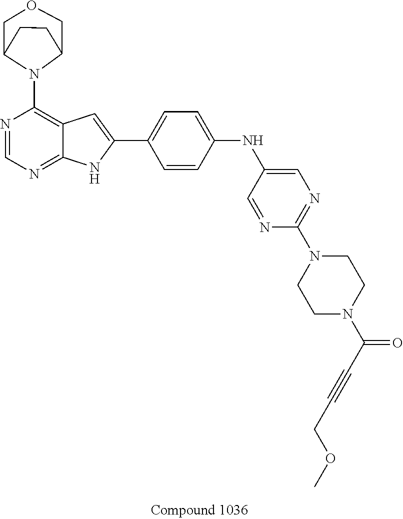 [SMILES: COCC#CC(=O)N1CCN(c2ncc(Nc3ccc(-c4cc5c(N6C7CCC6COC7)ncnc5[nH]4)cc3)cn2)CC1]

### Synthesis of 4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)aniline (32)

To a stirred solution of 8-(6-iodo-7-((2-(trimethylsilyl)ethoxy)methyl)-7H-pyrrolo[2,3-d]pyrimidin-4-yl)-3-oxa-8-azabicyclo[3.2.1]octane 30 (1 g, 2.06 mmol) and (4-aminophenyl)boronic acid hydrochloride 31 (713 mg, 4.11 mmol) in 1,4-dioxane (12 mL) was added K₂CO₃ (852 mg, 6.17 mmol) dissolved in water (3 mL). The reaction mixture was degassed with N₂ gas for 10 min, followed by added PdCl₂(dppf)·DCM (168 mg, 0.206 mmol) and stirred at 90° C. for 2 h. After completion of reaction (monitored by TLC), the reaction mixture was diluted with water (50 mL) and extracted with EtOAc (3×80 mL). The combined organic layer was washed with brine (2×50 mL), dried over Na₂SO₄, filtered and concentrated under reduced pressure. The crude was purified by flash column chromatography (silica gel 230-400 M, 12 g SNAP) using eluents 25-30% EtOAc in heptane to get the title compound as brown sticky solid 32 (730 mg, 76%).

¹H NMR (400 MHz, DMSO): δ 8.22 (s, 1H), 7.42 (d, J=8.8 Hz, 2H), 6.65-6.63 (m, 3H), 5.48 (s, 2H), 5.38 (s, 2H), 3.69-3.59 (m, 7H), 2.02-1.95 (m, 4H), 1.24 (m, 1H), 0.867-0.79 (m, 2H), −0.075 (s, 9H).

LCMS=[M+H]⁺: 452.09; Purity=97.34%.

### Synthesis of tert-butyl 4-(5-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)pyrimidin-2-yl)piperazine-1-carboxylate (34)

To a stirred solution of 4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)aniline 32 (720 mg, 1.6 mmol) and tert-butyl 4-(5-bromopyrimidin-2-yl)piperazine-1-carboxylate 33 (550 mg, 1.6 mmol) in 1,4-dioxane (15 mL) was added NaO^{t}Bu (231 mg, 2.4 mmol). The reaction mixture was degassed with N₂ gas for 10 min, followed by Pd₂(dba)₃ (147 mg, 0.16 mmol) and XPhos (76.2 mg, 0.16 mmol) were added to the reaction mixture and stirred at 90° C. for 2 h. After completion of reaction (monitored by TLC), the reaction mixture was diluted with water (50 mL) and extracted with EtOAc (3×80 mL). The combined organic layer was washed with brine (2×50 mL), dried over Na₂SO₄, filtered and concentrated under reduced pressure. The crude was purified by flash column chromatography (silica gel 230-400 M, 12 g SNAP) using eluents 30% EtOAc in heptane to get the title compound as brown sticky solid 34 (850 mg, 69%).

LCMS=[M+H]⁺: 714.46; Purity=94.02%.

### Synthesis of N-(4-(4-(3-oxa-8-azabicyclo[3.2.1]octan-8-yl)-7H-pyrrolo[2,3-d]pyrimidin-6-yl)phenyl)-2-(piperazin-1-yl)pyrimidin-5-amine (35)

To an ice cold stirred solution of tert-butyl 4-(5-((4-(4-(3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)pyrimidin-2-yl)piperazine-1-carboxylate 34 (850 mg, 1.19 mmol) in DCM (12 mL) was added TFA (6 mL) dropwise under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 1,4-dioxane (8 mL) and ethylenediamine (240 μL, 3.57 mmol) was added and stirred at 60° C. for 1 h. After completion of reaction (monitored by LCMS), the reaction was poured into ice cold water (50 mL) and extracted with 25% IPA in CHCl₃ (3×60 mL). The combined organic layer was dried over Na₂SO₄, filtered and concentrated under reduced pressure to get the title compound as a pale yellow solid 35 (560 mg; crude), which was used in next step directly without further purification.

LCMS=[M+H]⁺: 484.26; Purity=83.45%.

### Synthesis of 1-(4-(5-((4-(4-((1R,5S)-3-oxa-8-azabicyclo[3.2.1]octan-8-yl)-7H-pyrrolo[2,3-d]pyrimidin-6-yl)phenyl)amino)pyrimidin-2-yl)piperazin-1-yl)-4-methoxybut-2-yn-1-one (Compound 1036)

To an ice cold stirred solution of N-(4-(4-((1R,5S)-3-oxa-8-azabicyclo[3.2.1]octan-8-yl)-7H-pyrrolo[2,3-d]pyrimidin-6-yl)phenyl)-2-(piperazin-1-yl)pyrimidin-5-amine 35 (200 mg, 0.414 mmol) and 4-methoxybut-2-ynoic acid 36 (70.8 mg, 0.620 mmol) in DMF (5 mL) was added DIPEA (0.2 mL, 1.24 mmol) and T3P (0.5 mL, 0.827 mmol) under N₂ atmosphere. The reaction mixture was stirred at RT for 1 h. After completion of reaction (monitored by TLC), the reaction mixture was diluted with ice cold water (30 mL) and extracted with 25% IPA in CHCl₃ (3×30 mL). The combined organic layer was washed with ice cold brine (2×20 mL), dried over Na₂SO₄, filtered and concentrated under reduced pressure. The crude was purified by flash column chromatography (silica gel 230-400 M, 4 g SNAP) using eluents 5-10% MeOH in DCM to get Compound 1036 (77 mg, 32%).

¹H NMR (400 MHz, DMSO): § 12.01 (s, 1H), 8.34 (s, 2H), 8.13 (s, 1H), 7.97 (s, 1H), 7.72 (d, J=8.8 Hz, 2H), 6.88 (d, J=1.6 Hz, 1H), 6.84 (d, J=8.8 Hz, 2H), 4.87 (s, 2H), 4.34 (s, 2H), 3.79 (m, 4H), 3.74-3.70 (m, 3H), 3.67 (s, 1H), 3.62-3.59 (m, 4H), 2.03-1.95 (m, 4H). [3H merged in solvent residual peak]LCMS=[M+H]⁺: 580.26; Purity=95.04%.

### Example 125

### Compound 1037

### Preparation of (R)-4-((3-(4-hydroxybut-2-ynamido)piperidin-1-yl)methyl)-N-(4-(4-morpholino-7H-pyrrolo[2,3-d]pyrimidin-6-yl)phenyl)picolinamide (Compound 1037)

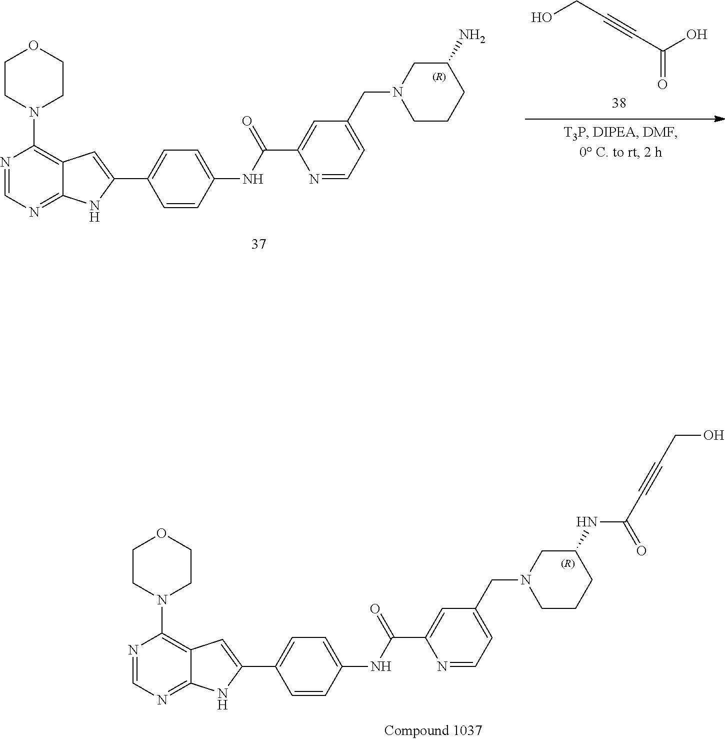

To an ice cold stirred solution of (R)-4-((3-aminopiperidin-1-yl)methyl)-N-(4-(4-morpholino-7H-pyrrolo[2,3-d]pyrimidin-6-yl)phenyl)picolinamide 37 (200 mg, 0.390 mmol) in DMF (6 mL) was added DIPEA (0.20 mL, 1.17 mmol) and stirred for 5 min. Then, 4-hydroxybut-2-ynoic acid 38 (50 mg, 0.468 mmol) and T3P (0.25 mL, 0.798 mmol) were added sequentially to the reaction mixture at same temperature under N₂ atmosphere. The reaction mixture was stirred at RT for 2 h. After completion of reaction (monitored by TLC), the reaction mixture was poured into ice cold water (50 mL) and extracted with EtOAc (3×50 mL). The combined organic layer was washed with ice cold brine (2×30 mL), dried over Na₂SO₄, filtered and concentrated under reduced pressure. The crude residue was purified by RP-HPLC [5-95% 5 mM Ammonium Bicarbonate in Water/ACN; Column X Select Phenyl Hexyl (19*250) 5μ] to get Compound 1037 (24 mg, 10.34%).

¹H NMR (400 MHz, DMSO-d₆): δ 12.21 (s, 1H), 10.76 (s, 1H), 8.68 (d, J=5.2 Hz, 1H), 8.58 (d, J=8.0 Hz, 1H), 8.18 (s, 1H), 8.10 (s, 1H), 8.00 (d, J=8.8 Hz, 2H), 7.91 (d, J=8.8 Hz, 2H), 7.61 (d, J=4.0 Hz, 1H), 7.16 (s, 1H), 5.49 (t, J=5.6 Hz, 1H), 4.19 (d, J=6.0 Hz, 2H), 3.88-3.86 (m, 4H), 3.76-3.73 (m, 5H), 3.64 (s, 2H), 2.77-2.75 (m, 1H), 2.66-2.63 (m, 1H), 1.95-1.86 (m, 2H), 1.78-1.63 (m, 2H), 1.50-1.47 (m, 1H), 1.21-1.19 (m, 1H).

LCMS=[M+H]⁺: 595.23; Purity=96.66%

### Example 126

### Compound 1040

### Preparation of 1-(4-(5-(4-(4-morpholino-7H-pyrrolo[2,3-d]pyrimidin-6-yl)benzyl)pyrimidin-2-yl)piperazin-1-yl)prop-2-en-1-one (Compound 1040)

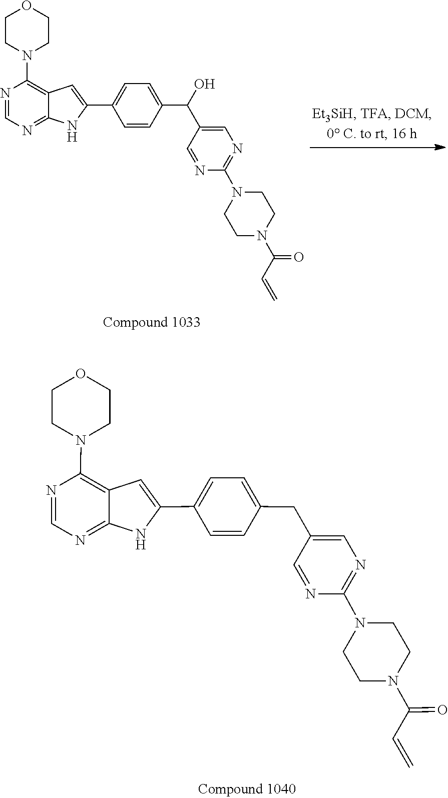 [SMILES: C=CC(=O)N1CCN(c2ncc(C(O)c3ccc(-c4cc5c(N6CCOCC6)ncnc5[nH]4)cc3)cn2)CC1 ; C=CC(=O)N1CCN(c2ncc(Cc3ccc(-c4cc5c(N6CCOCC6)ncnc5[nH]4)cc3)cn2)CC1]

To an ice cold stirred solution of 1-(4-(5-(hydroxy (4-(4-morpholino-7H-pyrrolo[2,3-d]pyrimidin-6-yl)phenyl)methyl)pyrimidin-2-yl)piperazin-1-yl)prop-2-en-1-one Compound 1030 (40 mg, 0.076 mmol) in DCM (3 mL) was added Et₃SiH (35 μL, 0.38 mmol) followed by TFA (50 μL, 0.60 mmol) under N₂ atmosphere.

The reaction mixture was stirred at RT for 16 h. After completion of reaction (monitored by TLC and LCMS), the reaction mixture was concentrated under reduced pressure. The resulting crude was purified by RP-HPLC [2-98% 0.1% TFA in Water/ACN; Column: SunFire C18 (19*250) 10μ] to get Compound 1040 (7 mg, 18%).

¹H NMR (400 MHz, DMSO): § 12.56 (s, 1H), 8.34 (s, 2H), 8.25 (s, 1H), 7.85 (d, J=8.4 Hz, 2H), 7.32 (d, J=8.4 Hz, 2H), 7.25 (s, 1H), 6.83 (dd, J=10.4 Hz, 16.8 Hz, 1H), 6.13 (dd, J=2.4 Hz, 16.4 Hz, 1H), 5.70 (dd, J=2.0 Hz, 10.4 Hz, 1H), 3.91-3.90 (m, 4H), 3.82 (s, 2H), 3.78-3.77 (m, 4H), 3.71-3.69 (m, 4H), 3.63-3.60 (m, 4H).

LCMS=[M+H]⁺: 511.21; Purity=95.79%.

### Example 127

### Compound 1050

### Preparation of N-((R)-1-(5-((4-(4-((1R,5S)-3-oxa-8-azabicyclo[3.2.1]octan-8-yl)-7H-pyrrolo[2,3-d]pyrimidin-6-yl)phenyl)amino)pyrimidin-2-yl)piperidin-3-yl)-2-(azetidin-1-ylmethyl)acrylamide (Compound 1050)

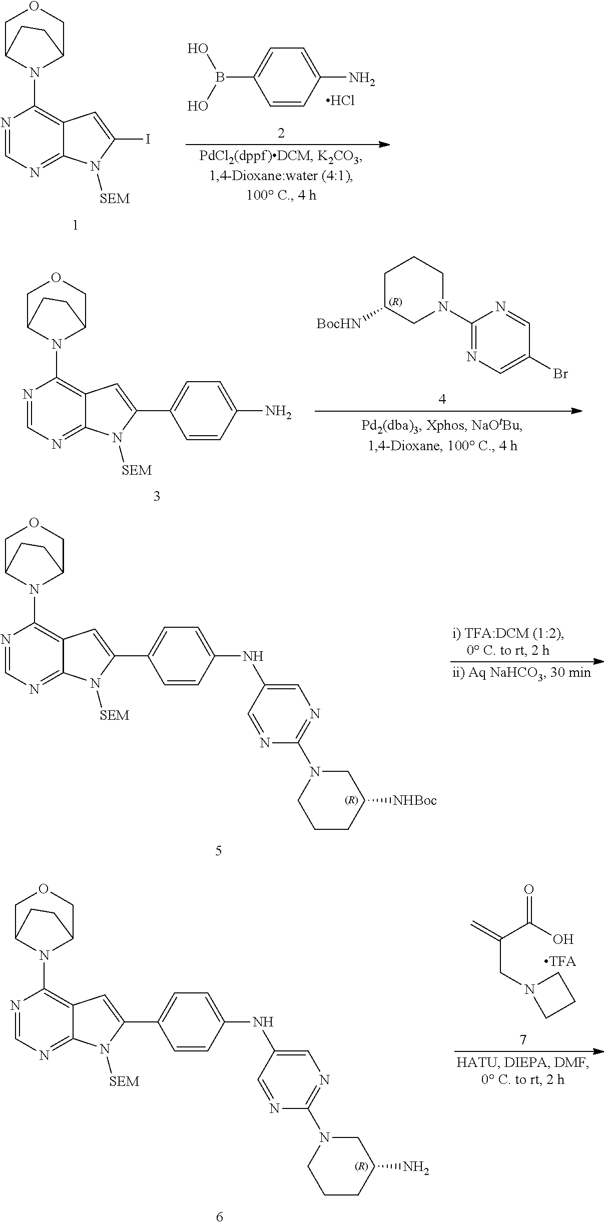

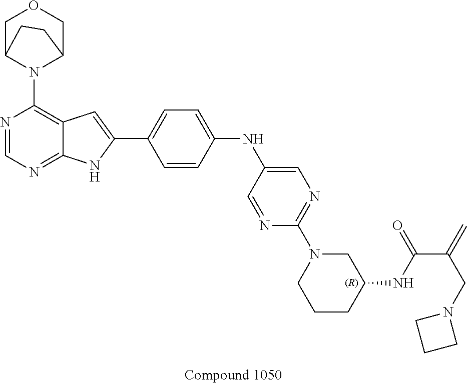

### Synthesis of 4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)aniline (3)

To a stirred solution of (1R,5S)-8-(6-iodo-7-((2-(trimethylsilyl)ethoxy)methyl)-7H-pyrrolo[2,3-d]pyrimidin-4-yl)-3-oxa-8-azabicyclo[3.2.1]octane 1 (1 g, 2.06 mmol) and (4-aminophenyl) boronic acid·HCl ₂ (713 mg, 4.11 mmol) in 1,4-Dioxane (12 mL) and water (3 mL) was added K₂CO₃ (852 mg, 6.17 mmol) at RT. The resulting reaction mixture was degassed with argon gas for 15 min. Then, PdCl₂(dppf)·DCM (168 mg, 0.206 mmol) was added and the resulting reaction mixture was stirred at 100° C. for 4 h. After the completion of reaction (monitored by TLC), the reaction mixture was diluted with water (50 mL) and extracted with EtOAc (3×80 mL). The combined organic layer was washed with brine solution (2×50 mL), dried over Na₂SO₄, filtered and evaporated under reduced pressure. The crude was purified by flash column chromatography (silica gel 230-400 M, 40 g SNAP) using eluents 30-40% EtOAc in heptane to get the title compound as a brown solid 3 (750 mg, 74%).

LCMS=[M+H]⁺: 452.10; Purity=80%.

### Synthesis of tert-butyl ((R)-1-(5-((4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)pyrimidin-2-yl)piperidin-3-yl)carbamate (5)

To a stirred solution of 4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)aniline 3 (700 mg, 1.55 mmol) and tert-butyl (R)-(1-(5-bromopyrimidin-2-yl)piperidin-3-yl)carbamate 4 (692 mg, 1.94 mmol) in 1,4-Dioxane (10 mL) was added NaO^{t}Bu (225 mg, 2.32 mmol) at RT. The resulting reaction mixture was degassed with N₂ gas for 10 min, followed by added XPhos (74 mg, 0.155 mmol) and Pd₂(dba)₃ (125 mg, 0.155 mmol) and stirred at 100° C. for 4 h. After completion of reaction (monitored by TLC and LCMS), the reaction mixture was diluted with water (80 mL) and extracted with EtOAc (3×80 mL). The combined organic layer was dried over Na₂SO₄, filtered and evaporated under reduced pressure. The crude was purified by flash column chromatography (silica gel 100-200 M, 12 g SNAP) using eluents 50-60% EtOAc in heptane to get the title compound as an off white solid 5 (760 g, 67%).

LCMS=[M+H]⁺: 728.11; Purity=80%

### Synthesis of N-(4-(4-((1R,5S)-3-oxa-8-azabicyclo[3.2.1]octan-8-yl)-7H-pyrrolo[2,3-d]pyrimidin-6-yl)phenyl)-2-((R)-3-aminopiperidin-1-yl)pyrimidin-5-amine (6)

To an ice cold stirred solution of tert-butyl ((R)-1-(5-((4-(4-((1R,5S)-3-oxa-8-azabicyclo[3.2.1]octan-8-yl)-7-((2-(trimethylsilyl)ethoxy)methyl)-7H-pyrrolo[2,3-d]pyrimidin-6-yl)phenyl)amino)pyrimidin-2-yl)piperidin-3-yl)carbamate 5 (700 mg, 0.962 mmol) in DCM (10 mL) was added TFA (5 mL) dropwise under N2 atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 25% IPA in CHCl₃ (10 mL), followed by sat. NaHCO₃ (20 mL) was added at RT. The resulting reaction mixture was stirred at RT for 30 min. After completion of reaction (monitored by LCMS), the reaction was extracted with 25% IPA in CHCl₃ (3×20 mL). The combined organic layer was dried over Na₂SO₄, filtered and concentrated under reduced pressure to get the title compound as a yellow solid 6 (430 mg; crude), which was used in next step directly without further purification.

LCMS=[M+H]⁺: 498.10; Purity=70%.

### Synthesis of N-((R)-1-(5-((4-(4-((1R,5S)-3-oxa-8-azabicyclo[3.2.1]octan-8-yl)-7H-pyrrolo[2,3-d]pyrimidin-6-yl)phenyl)amino)pyrimidin-2-yl)piperidin-3-yl)-2-(azetidin-1-ylmethyl)acrylamide (Compound 1050)

To an ice cold stirred solution of N-(4-(4-((1R,5S)-3-oxa-8-azabicyclo[3.2.1]octan-8-yl)-7H-pyrrolo[2,3-d]pyrimidin-6-yl)phenyl)-2-((R)-3-aminopiperidin-1-yl)pyrimidin-5-amine 6 (200 mg, 0.402 mmol) and 2-(azetidin-1-ylmethyl)acrylic acid. TFA 7 (308 mg, 1.21 mmol) in DMF (5 mL) was added DIPEA (0.42 mL, 2.41 mmol) under N₂ atmosphere. Then, HATU (7 (230 mg, 0.603 mmol) was added to the reaction mixture at 0° C. and stirred at RT for 2 h. After completion of reaction (monitored by TLC and LCMS), the reaction mixture was poured into ice cold water (50 mL) and extracted with 25% IPA in CHCl₃ (3×50 mL). The combined organic layer was washed with ice cold brine (2×50 mL), dried over Na₂SO₄, filtered and concentrated under reduced pressure. The crude was purified by RP-HPLC [5-95% 5 mM Ammonium Bicarbonate in water/ACN; Column: X Bridge C18 (19*250) 10μ] to get Compound 1050 (22 mg, 9%).

¹H NMR (400 MHz, DMSO-d₆): δ 12.01 (s, 1H), 9.11 (d, J=7.6 Hz, 1H), 8.28 (s, 2H), 8.14 (s, 1H), 7.88 (s, 1H), 7.70 (d, J=8.4 Hz, 2H), 6.88 (d, J=1.6 Hz, 1H), 6.76 (d, J=8.4 Hz, 2H), 5.80 (d, J=1.6 Hz, 1H), 5.38 (s, 1H), 4.87 (brs, 2H), 3.89-3.86 (m, 2H), 3.79-3.75 (m, 2H), 3.73-3.72 (m, 2H), 3.70-3.67 (m, 2H), 3.11 (q, J=13.2 Hz, 2H), 3.02 (q, J=6.8 Hz, 2H), 2.94 (q, J=6.8 Hz, 2H), 2.02-1.90 (m, 4H), 1.86-1.80 (m, 3H), 1.75-1.67 (m, 2H), 1.60-1.55 (m, 1H). [1H is not appeared may merged in solvent peak]LCMS=[M+H]⁺: 621.42; Purity=95.08%.

### Example 128

### Compound 1051

### Preparation of (R)-4-hydroxy-N-(1-(5-((4-(4-morpholino-7H-pyrrolo[2,3-d]pyrimidin-6-yl)phenyl)amino)pyrimidin-2-yl)piperidin-3-yl)but-2-ynamide (Compound 1051)

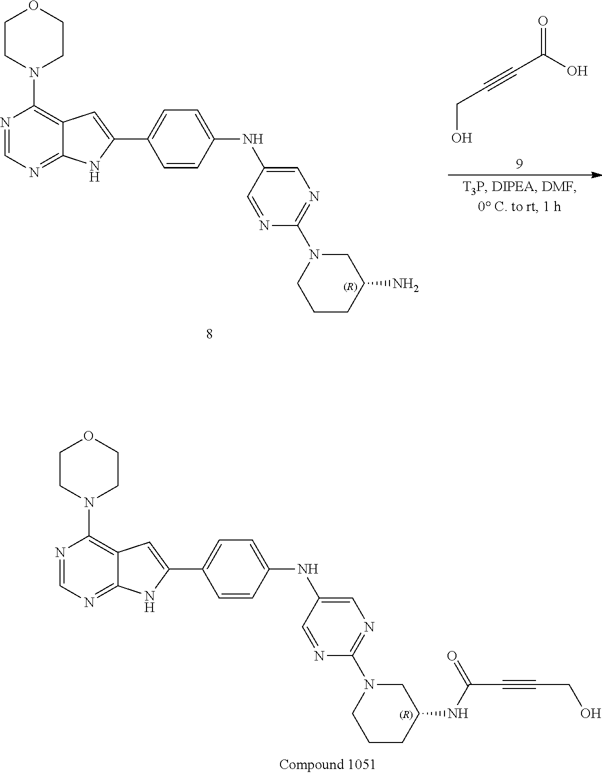

To a stirred solution of (R)-2-(3-aminopiperidin-1-yl)-N-(4-(4-morpholino-7H-pyrrolo[2,3-d]pyrimidin-6-yl)phenyl)pyrimidin-5-amine 8 (150 mg, 0.318 mmol) and 4-hydroxybut-2-ynoic acid 9 (47.7 mg, 0.477 mmol) in DMF (3 mL) were added DIPEA (0.17 mL, 0.954 mmol) and T3P (304 mg, 0.477 mmol; 50 wt. % in ethyl acetate) at 0° C. The resulting reaction mixture was stirred at RT for 1 h. After completion of reaction (monitored by TLC and LCMS), the reaction mixture was poured into ice cold water (20 mL) and extracted with 25% IPA in CHCl₃ (3×50 mL). The combined organic layer was dried over Na₂SO₄, filtered and evaporated under reduced pressure. The crude was purified by RP-HPLC [2-98% 0.1% TFA in water/ACN; Column: Sunfire C18 (19*250) 10μ] to get Compound 1051 (27 mg, 12%).

¹H NMR (400 MHz, DMSO): & 12.59 (s, 1H), 8.73 (d, J=8.0 Hz, 1H), 8.29 (s, 2H), 8.26 (s, 1H), 7.98 (brs, 1H), 7.72 (d, J=8.4 Hz, 2H), 7.11 (brs, 1H), 6.83 (d, J=8.4 Hz, 2H), 4.48-4.44 (m, 1H), 4.36-4.33 (m, 1H), 4.22 (s, 2H), 4.91-4.90 (m, 4H), 3.79-3.77 (m, 4H), 3.69-3.65 (m, 2H), 2.99-2.94 (m, 1H), 2.88-2.82 (m, 1H), 1.85-1.81 (m, 1H), 1.77-1.75 (m, 1H), 1.53-1.43 (m, 2H).

LCMS=[M+H]⁺: 554.31; Purity=99.8%.

### Example 129

### Compound 1053

### Preparation of (R)—N-(4-(4-morpholino-7H-pyrrolo[2,3-d]pyrimidin-6-yl)phenyl)-4-((3-(2-((4-nicotinoylpiperazin-1-yl)methyl)acrylamido)piperidin-1-yl)methyl)picolinamide (Compound 1053)

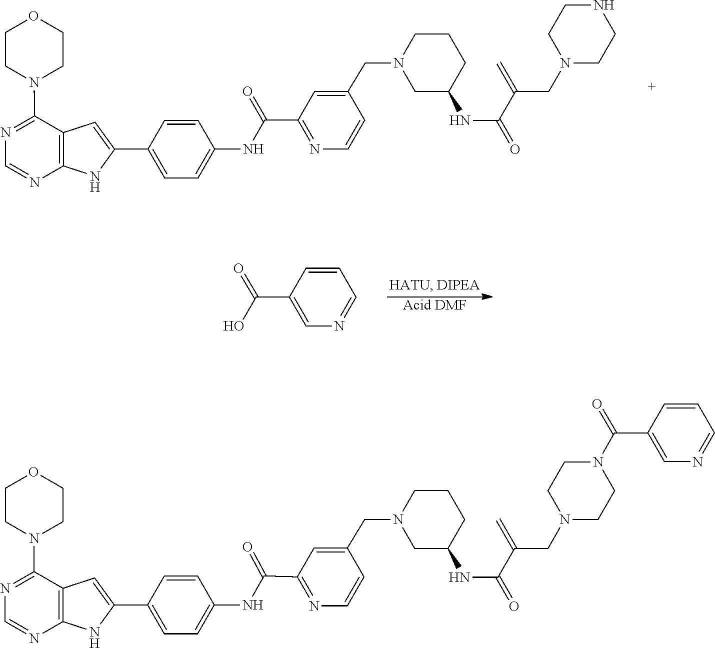 [SMILES: C=C(CN1CCNCC1)C(=O)N[C@@H]1CCCN(Cc2ccnc(C(=O)Nc3ccc(-c4cc5c(N6CCOCC6)ncnc5[nH]4)cc3)c2)C1 ; O=C(O)c1cccnc1 ; "Acid DMF" ; C=C(CN1CCN(C(=O)c2cccnc2)CC1)C(=O)N[C@@H]1CCCN(Cc2ccnc(C(=O)Nc3ccc(-c4cc5c(N6CCOCC6)ncnc5[nH]4)cc3)c2)C1]

N,N-Diisopropylethylamine (0.026 mL, 0.150 mmol) was added to a solution of O-(7-azabenzotriazol-1-yl)-N,N,N′,N′-tetramethyluronium hexafluorophosphate (9.3 mg, 0.0243 mmol) and nicotinic acid (2.8 mg, 0.0225 mmol) in N,N-dimethylformamide (1 mL). After 5 minutes (R)—N-(4-(4-morpholino-7H-pyrrolo[2,3-d]pyrimidin-6-yl)phenyl)-4-((3-(2-(piperazin-1-ylmethyl)acrylamido)piperidin-1-yl)methyl)picolinamide trihydrochloride (15 mg, 0.0187 mmol) was added. After 30 minutes the reaction was diluted with ethyl acetate (20 mL), washed with 5% lithium chloride (3×10 mL) saturated sodium bicarbonate (2×10 mL) and ammonium chloride (10 mL). The organic phase was dried over sodium sulfate and the solvent was removed under reduced pressure. The residue was subjected to preparative HPLC(5-85% acetonitrile/water, 0.1% trifluoroacetic acid). The fractions containing product were combined and subjected to lyophilization providing, (R)—N-(4-(4-morpholino-7H-pyrrolo[2,3-d]pyrimidin-6-yl)phenyl)-4-((3-(2-((4-nicotinoylpiperazin-1-yl)methyl)acrylamido)piperidin-1-yl)methyl)picolinamide trifluoroacetate (Compound 1053)(5.6 mg, 0.00634 mmol, 33% yield).

LCMS=[M+H]⁺: 770.40; Purity=95%

¹H NMR (400 MHz, MeOD) δ 8.74 (d, J=5.0 Hz, 1H), 8.56 (d, J=4.3 Hz, 2H), 8.30 (s, 1H), 8.21 (s, 1H), 7.86 (t, J=8.7 Hz, 3H), 7.77 (d, J=8.4 Hz, 2H), 7.67 (d, J=4.9 Hz, 1H), 7.48-7.40 (m, 1H), 7.18 (s, 1H), 6.20 (s, 1H), 5.92 (s, 1H), 4.42 (m, 2H), 4.11 (m, 1H), 3.99 (t, J=4.9 Hz, 4H), 3.89-3.80 (m, 7H), 3.80-3.60 (m, 3H), 3.37 (m, 3H), 3.15 (m, 5H), 1.97 (m, 2H), 1.82 (m, 1H), 1.62 (m, 1H).

### Example 130

### Compound 1054

### Preparation of (R)-4-((3-(2-((4-acetylpiperazin-1-yl)methyl)acrylamido)piperidin-1-yl)methyl)-N-(4-(4-morpholino-7H-pyrrolo[2,3-d]pyrimidin-6-yl)phenyl)picolinamide (Compound 1054)

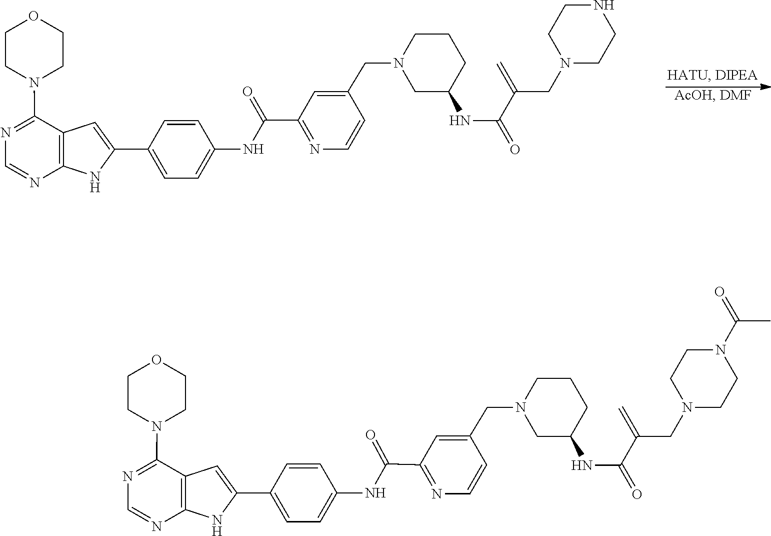

N,N-Diisopropylethylamine (0.026 mL, 0.150 mmol) was added to a solution of O-(7-azabenzotriazol-1-yl)-N,N,N′,N′-tetramethyluronium hexafluorophosphate (9.3 mg, 0.0243 mmol) and acetic acid (1.3 mg, 0.0225 mmol) in N,N-dimethylformamide (1 mL). After 5 minutes (R)—N-(4-(4-morpholino-7H-pyrrolo[2,3-d]pyrimidin-6-yl)phenyl)-4-((3-(2-(piperazin-1-ylmethyl)acrylamido)piperidin-1-yl)methyl)picolinamide trihydrochloride (15 mg, 0.0187 mmol) was added. After 30 minutes the reaction was diluted with ethyl acetate (20 mL). The mixture was washed with 5% lithium chloride (3×10 mL) saturated sodium bicarbonate (2×10 mL) and ammonium chloride (10 mL). The organic phase was dried over sodium sulfate and the solvent was removed under reduced pressure. The residue was subjected to preparative HPLC (5-85% acetonitrile/water, 0.1% trifluoroacetic acid). The fractions containing product were combined and subjected to lyophilization providing, (R)-4-((3-(2-((4-acetylpiperazin-1-yl)methyl)acrylamido)piperidin-1-yl)methyl)-N-(4-(4-morpholino-7H-pyrrolo[2,3-d]pyrimidin-6-yl)phenyl)picolinamide trifluoroacetate (Compound 1054)(5.7 mg, 0.00694 mmol, 37%).

LCMS=[M+H]⁺: 707.40; Purity=95%

¹H NMR (400 MHz, MeOD) δ 8.74 (d, J=4.9 Hz, 1H), 8.30 (s, 1H), 8.20 (d, J=1.7 Hz, 1H), 7.87 (d, J=8.1 Hz, 2H), 7.78 (d, J=8.3 Hz, 2H), 7.66 (d, J=5.0 Hz, 1H), 7.18 (s, 1H), 6.21 (s, 1H), 5.92 (s, 1H), 4.39 (m, 2H), 4.11 (m, 1H), 3.99 (t, J=4.9 Hz, 4H), 3.83 (m, 6H), 3.68 (m, 4H), 3.54-3.25 (m, 2H), 2.96-3.17 (m, 5H), 2.83 (m, 1H), 2.00 (s, 3H), 1.95 (m, 2H), 1.81 (m, 1H), 1.60 (m, 1H).

### Example 131

### Compound 1058

### Preparation of 4-(((R)-3-(2-((4-(acetyl-L-valyl)piperazin-1-yl)methyl)acrylamido)piperidin-1-yl)methyl)-N-(4-(4-morpholino-7H-pyrrolo[2,3-d]pyrimidin-6-yl)phenyl)picolinamide (Compound 1058)

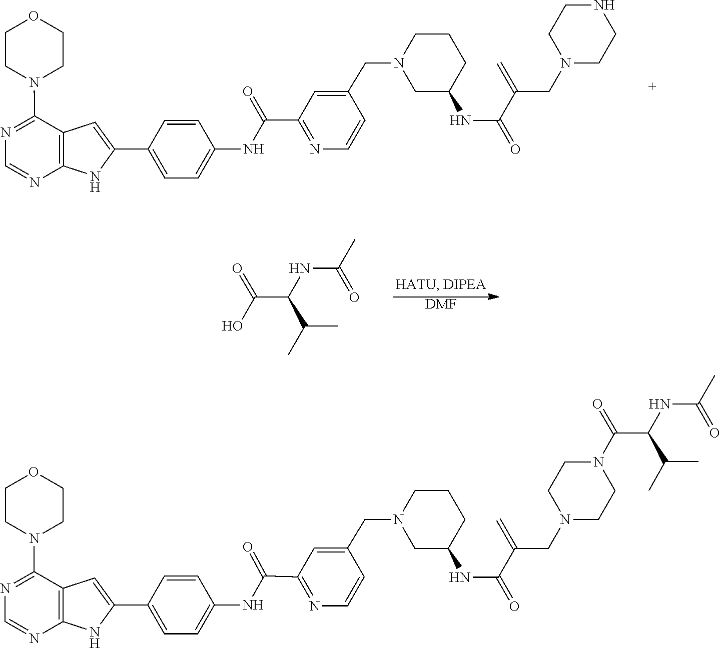

N,N-Diisopropylethylamine (0.045 mL, 0.258 mmol) was added to a solution of O-(7-azabenzotriazol-1-yl)-N,N,N′,N′-tetramethyluronium hexafluorophosphate (16 mg, 0.0420 mmol) and acetyl-L-valine (6.1 mg, 0.0388 mmol) in N,N-dimethylformamide (1 mL). After 5 min (R)—N-(4-(4-morpholino-7H-pyrrolo[2,3-d]pyrimidin-6-yl)phenyl)-4-((3-(2-(piperazin-1-ylmethyl)acrylamido)piperidin-1-yl)methyl)picolinamide trihydrochloride (25 mg, 0.0323 mmol) was added. After 20 minutes the reaction was diluted with ethyl acetate (20 mL). The mixture was washed with 5% lithium chloride (3×10 mL), saturated sodium bicarbonate (2×10 mL) and saturated ammonium chloride (10 mL). The organic phase was dried over sodium sulfate and the solvent was removed under reduced pressure. The residue was subjected to preparative HPLC(5-85% acetonitrile/water, 0.1% trifluoroacetic acid). The fractions containing product were combined and subjected to lyophilization, providing 4-(((R)-3-(2-((4-(acetyl-L-valyl)piperazin-1-yl)methyl)acrylamido)piperidin-1-yl)methyl)-N-(4-(4-morpholino-7H-pyrrolo[2,3-d]pyrimidin-6-yl)phenyl)picolinamide trifluoroacetate (Compound 1058)(3.9 mg, 0.00403 mmol, 12%).

LCMS=[M+H]⁺: 806.50; Purity=94%

¹H NMR (400 MHz, MeOD) δ 8.87 (d, J=5.0 Hz, 1H), 8.42 (s, 1H), 8.33 (s, 1H), 8.00 (d, J=8.4 Hz, 2H), 7.91 (d, J=8.4 Hz, 2H), 7.78 (d, J=5.0 Hz, 1H), 7.32 (s, 1H), 6.33 (s, 1H), 6.05 (s, 1H), 4.55 (m, 1H), 4.52 (m, 2H), 4.23 (m, 1H), 4.11 (m, 4H), 3.96 (m, 6H), 3.85 (m, 2H), 3.71-3.41 (m, 2H), 3.32-3.20 (m, 3H), 3.20-2.91 (m, 5H), 2.09 (m, 2H), 2.05 (m, 1H), 2.00 (s, 3H), 1.93 (m, 1H), 1.72 (m, 1H), 0.99 (d, J=6.7 Hz, 3H), 0.94 (d, J=6.8 Hz, 3H).

### Example 132

### Compound 1059

### Preparation of 4-(((R)-3-(2-((4-(acetyl-L-alanyl)piperazin-1-yl)methyl)acrylamido)piperidin-1-yl)methyl)-N-(4-(4-morpholino-7H-pyrrolo[2,3-d]pyrimidin-6-yl)phenyl)picolinamide (Compound 1059)

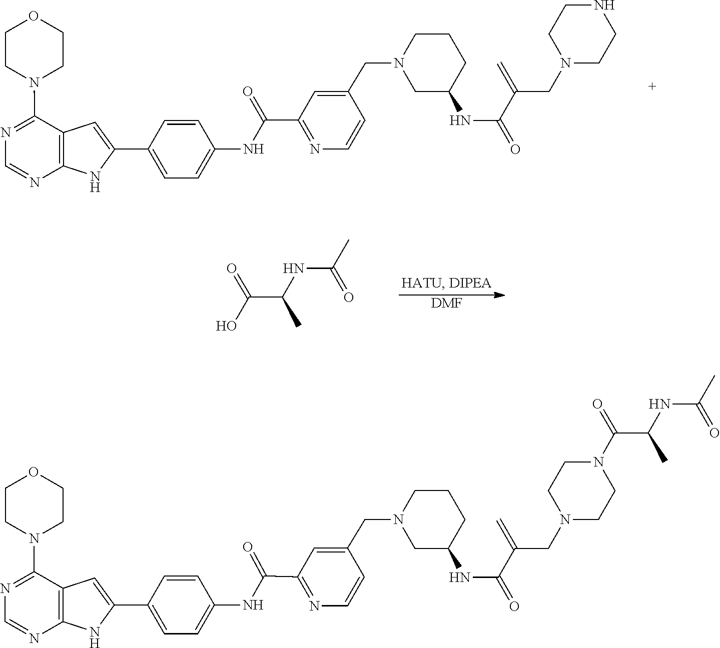

N,N-Diisopropylethylamine (0.045 mL, 0.258 mmol) was added to a solution of O-(7-azabenzotriazol-1-yl)-N,N,N′,N′-tetramethyluronium hexafluorophosphate (16 mg, 0.0420 mmol) and acetyl-L-alanine (6.1 mg, 0.0388 mmol) in N,N-dimethylformamide (1 mL). After 5 min (R)—N-(4-(4-morpholino-7H-pyrrolo[2,3-d]pyrimidin-6-yl)phenyl)-4-((3-(2-(piperazin-1-ylmethyl)acrylamido)piperidin-1-yl)methyl)picolinamide trihydrochloride (25 mg, 0.0323 mmol) was added. After 20 minutes the reaction was diluted with ethyl acetate (20 mL). The mixture was washed with 5% lithium chloride (3×10 mL), saturated sodium bicarbonate (2×10 mL) and saturated ammonium chloride (10 mL). The organic phase was dried over sodium sulfate and the solvent was removed under reduced pressure. The residue was subjected to preparative HPLC(5-85% acetonitrile/water, 0.1% trifluoroacetic acid). The fractions containing product were combined and subjected to lyophilization, providing 4-(((R)-3-(2-((4-(acetyl-L-alanyl)piperazin-1-yl)methyl)acrylamido)piperidin-1-yl)methyl)-N-(4-(4-morpholino-7H-pyrrolo[2,3-d]pyrimidin-6-yl)phenyl)picolinamide trifluoroacetate (Compound 1059)(3.9 mg, 0.00415 mmol, 12%).

LCMS=[M+H]⁺: 778.50; Purity=95%

¹H NMR (400 MHz, MeOD) δ 8.87 (d, J=4.9 Hz, 1H), 8.42 (s, 1H), 8.33 (s, 1H), 8.00 (d, J=8.4 Hz, 2H), 7.91 (d, J=8.4 Hz, 2H), 7.79 (d, J=5.0 Hz, 1H), 7.32 (s, 1H), 6.34 (s, 1H), 6.06 (s, 1H), 4.78 (m, H), 4.53 (m, 2H), 4.23 (m, 1H), 4.12 (t, J=4.9 Hz, 4H), 3.96 (m, 8H), 3.81 (m, 1H), 3.54 (m, 1H), 3.47 (m, 1H), 3.32-3.23 (m, 2H), 2.90-3.23 (m, 5H), 2.07 (m, 2H), 1.97 (s, 3H), 1.92 (m, 1H), 1.73 (m, 1H), 1.30 (d, J=7.0 Hz, 3H).

### Example 133

### Compound 1062

### Preparation of 1-(4-(5-((4-(4-morpholino-7H-pyrrolo[2,3-d]pyrimidin-6-yl)phenyl) sulfonyl)pyrimidin-2-yl)piperazin-1-yl)prop-2-en-1-one (Compound 1062)

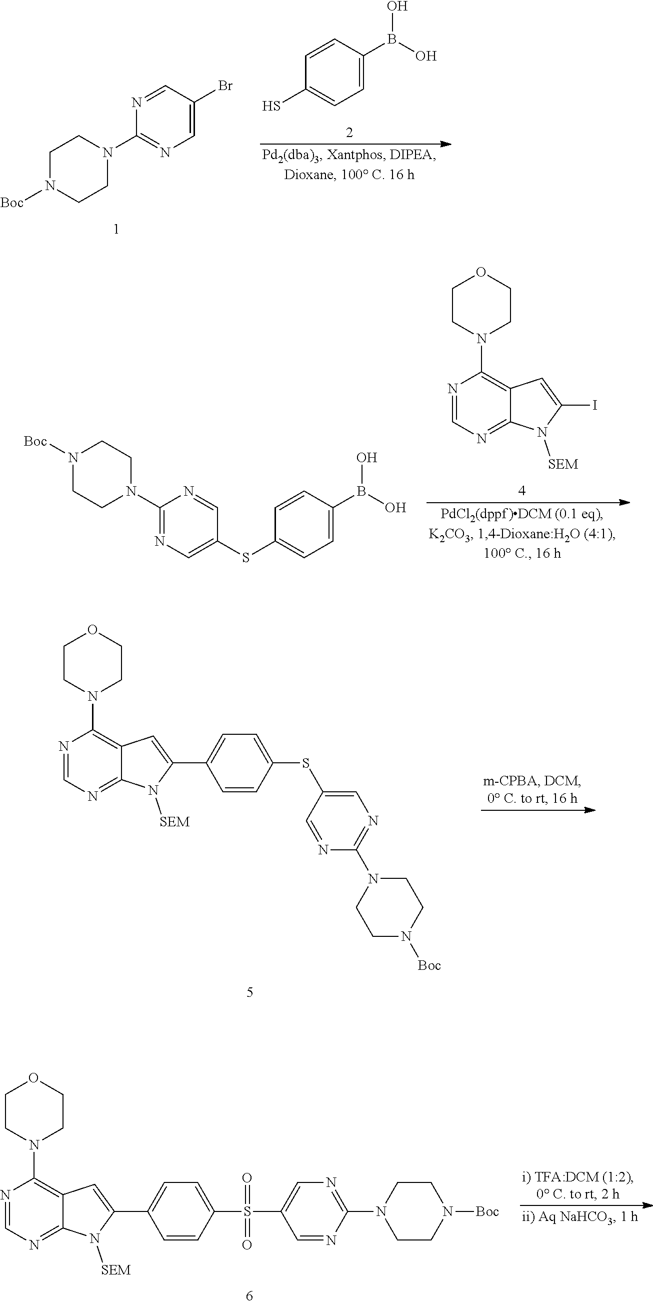 [SMILES: CC(C)(C)OC(=O)N1CCN(c2ncc(Br)cn2)CC1 ; OB(O)c1ccc(S)cc1 ; CC(C)(C)OC(=O)N1CCN(c2ncc(Sc3ccc(B(O)O)cc3)cn2)CC1 ; C[Si](C)(C)CCOCn1c(I)cc2c(N3CCOCC3)ncnc21 ; CC(C)(C)OC(=O)N1CCN(c2ncc(Sc3ccc(-c4cc5c(N6CCOCC6)ncnc5n4COCC[Si](C)(C)C)cc3)cn2)CC1 ; CC(C)(C)OC(=O)N1CCN(c2ncc(S(=O)(=O)c3ccc(-c4cc5c(N6CCOCC6)ncnc5n4COCC[Si](C)(C)C)cc3)cn2)CC1]

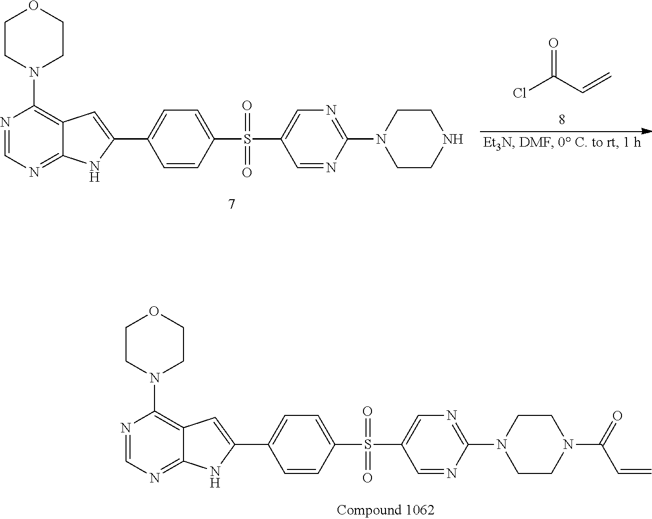

### Synthesis of (4-((2-(4-(tert-butoxycarbonyl)piperazin-1-yl)pyrimidin-5-yl)thio)phenyl) boronic acid (3)

To a stirred solution of tert-butyl 4-(5-bromopyrimidin-2-yl)piperazine-1-carboxylate 1 (3 g, 8.74 mmol) and (4-mercaptophenyl) boronic acid 2 (2.1 g, 13.1 mmol) in 1,4-Dioxane (30 mL) was added DIPEA (3.0 mL, 17.5 mmol) at RT. The resulting reaction mixture was degassed with N₂ gas for 10 min, followed by added Xanthphos (506 mg, 0.874 mmol) and Pd₂(dba)₃ (800 mg, 0.874 mmol) and stirred at 100° C. for 16 h. After completion of reaction (monitored by TLC and LCMS), the reaction mixture was diluted with water (100 mL) and extracted with EtOAc (3×100 mL). The combined organic layer was dried over Na₂SO₄, filtered and evaporated under reduced pressure. The crude was purified by flash column chromatography (silica gel 100-200 M, 40 g SNAP) using eluents 30-40% EtOAc in heptane to get the title compound as an yellow solid 3 (2.2 g, 60%).

LCMS=[M+H]⁺: 417.05; Purity=70%

### Synthesis of tert-butyl 4-(5-((4-(4-morpholino-7-((2-(trimethylsilyl)ethoxy)methyl)-7H-pyrrolo[2,3-d]pyrimidin-6-yl)phenyl)thio)pyrimidin-2-yl)piperazine-1-carboxylate (5)

To a stirred solution of (4-((2-(4-(tert-butoxycarbonyl)piperazin-1-yl)pyrimidin-5-yl)thio)phenyl) boronic acid 3 (1.5 g, 3.26 mmol) and 4-(6-iodo-7-((2-(trimethylsilyl)ethoxy)methyl)-7H-pyrrolo[2,3-d]pyrimidin-4-yl)morpholine 4 (2.0 g, 4.89 mmol) in 1,4-Dioxane (16 mL) and H₂O (4 mL) was added K₂CO₃ (1.35 g, 9.75 mmol) at RT. The resulting reaction mixture was degassed with argon gas for 15 min. Then was added PdCl₂(dppf)·DCM (265 mg, 0.326 mmol) and the resulting reaction mixture was stirred at 100° C. for 16 h. After the completion of reaction (monitored by TLC), the reaction mixture was diluted with water (50 mL) and extracted with EtOAc (3×80 mL). The combined organic layer was washed with brine solution (2×50 mL), dried over Na₂SO₄, filtered and evaporated under reduced pressure. The crude was purified by flash column chromatography (silica gel 230-400 M, 12 g SNAP) using eluents 30-40% EtOAc in heptane to get the title compound as a brown solid 5 (1.3 g, 45%).

LCMS=[M+H]⁺: 705.46; Purity=95%.

### Synthesis of tert-butyl 4-(5-((4-(4-morpholino-7-((2-(trimethylsilyl)ethoxy)methyl)-7H-pyrrolo[2,3-d]pyrimidin-6-yl)phenyl) sulfonyl)pyrimidin-2-yl)piperazine-1-carboxylate (6)

To a stirred solution of tert-butyl 4-(5-((4-(4-morpholino-7-((2-(trimethylsilyl)ethoxy)methyl)-7H-pyrrolo[2,3-d]pyrimidin-6-yl)phenyl)thio)pyrimidin-2-yl)piperazine-1-carboxylate 5 (1 g, 1.42 mmol) in DCM (20 mL) was added m-CPBA (980 mg, 4.26 mmol) portion wise at 0° C. under N₂ atmosphere. The resulting reaction mixture was stirred at RT for 16 h. After the completion of reaction (monitored by TLC), the reaction mixture was diluted with water (50 mL) and extracted with DCM (3×80 mL). The combined organic layer was washed with brine solution (2×50 mL), dried over Na₂SO₄, filtered and evaporated under reduced pressure. The crude was purified by flash column chromatography (silica gel 230-400 M, 12 g SNAP) using eluents 3-5% MeOH in DCM to get the title compound as a light yellow solid 6 (500 mg, 47%).

LCMS=[M+H]⁺: 737.42; Purity=70%.

### Synthesis of 4-(6-(4-((2-(piperazin-1-yl)pyrimidin-5-yl) sulfonyl)phenyl)-7H-pyrrolo[2,3-d]pyrimidin-4-yl)morpholine (7)

To an ice cold stirred solution of tert-butyl 4-(5-((4-(4-morpholino-7-((2-(trimethylsilyl)ethoxy)methyl)-7H-pyrrolo[2,3-d]pyrimidin-6-yl)phenyl) sulfonyl)pyrimidin-2-yl)piperazine-1-carboxylate 6 (500 mg, 0.679 mmol) in DCM (6 mL) was added TFA (3 mL) dropwise under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 25% IPA in CHCl₃ (10 mL) and followed by saturated aq. NaHCO₃ (10 mL) was added and stirred at RT for 1 h. After completion of reaction (monitored by LCMS), the reaction was poured into ice-cold water (50 mL) and extracted with 25% IPA in CHCl₃ (3×50 mL). The combined organic layer was dried over Na₂SO₄, filtered and concentrated under reduced pressure to get the title compound as a pale yellow solid 7 (350 mg; crude), which was used in next step directly without further purification.

LCMS=[M−H]: 505.04; Purity=65%.

### Synthesis of 1-(4-(5-((4-(4-morpholino-7H-pyrrolo[2,3-d]pyrimidin-6-yl)phenyl) sulfonyl)pyrimidin-2-yl)piperazin-1-yl)prop-2-en-1-one (Compound 1062)

To an ice cold stirred solution of 4-(6-(4-((2-(piperazin-1-yl)pyrimidin-5-yl) sulfonyl)phenyl)-7H-pyrrolo[2,3-d]pyrimidin-4-yl)morpholine 7 (350 mg, 0.691 mmol) in DMF (5 mL) was added Et₃N (0.3 mL, 2.07 mmol) and stirred for 5 min. Then, a solution of acryloyl chloride 8 (67 μL, 0.829 mmol) in DMF (1 mL) was added dropwise to the reaction mixture at same temperature under N₂ atmosphere. The reaction mixture was stirred at RT for 1 h. After completion of reaction (monitored by TLC), the reaction mixture was poured into ice-cold water (50 mL), obtained precipitate was filtered through sintered funnel, washed with Et₂O and dried under reduced pressure. The crude residue was purified by RP-HPLC [5-95% 0.1% TFA in Water/ACN; Column: X Bridge C18 (19*250) 10μ] to get Compound 1062 (12 mg, 3%).

¹H NMR (400 MHz, DMSO-d₆): δ 12.61 (s, 1H), 8.88 (s, 2H), 8.24 (s, 1H), 8.13 (d, J=8.4 Hz, 2H), 8.02 (d, J=8.8 Hz, 2H), 7.49 (s, 1H), 6.81 (dd, J=16.8 Hz, 10.4 Hz, 1H), 6.13 (dd, J=16.4 Hz, 2.4 Hz, 1H), 5.71 (dd, J=10.4 Hz, 2.4 Hz, 1H), 3.93-3.90 (m, 4H), 3.86-3.81 (m, 4H), 3.77-3.74 (m, 4H), 3.66-3.58 (m, 4H).

LCMS=[M+H]⁺: 561.35; Purity=95.44%

### Example 134

### Compound 1064

### Preparation of (R)-4-((3-(2-((4-(acetylglycyl)piperazin-1-yl)methyl)acrylamido)piperidin-1-yl)methyl)-N-(4-(4-morpholino-7H-pyrrolo[2,3-d]pyrimidin-6-yl)phenyl)picolinamide (Compound X)

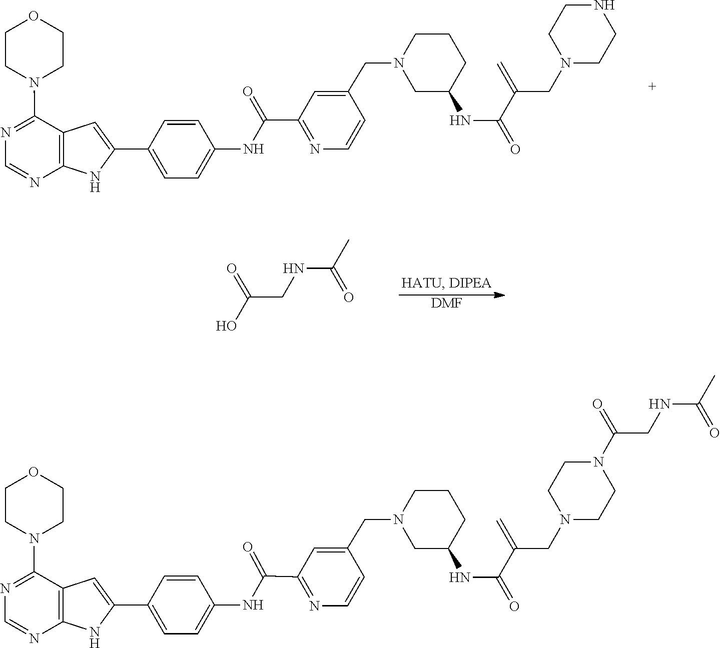

N,N-Diisopropylethylamine (0.045 mL, 0.258 mmol) was added to a solution of O-(7-azabenzotriazol-1-yl)-N,N,N′. N′-tetramethyluronium hexafluorophosphate (16 mg, 0.0420 mmol) and acetylglycine (4.5 mg, 0.0388 mmol) in N,N-dimethylformamide (1 mL). After 5 minutes (R)—N-(4-(4-morpholino-7H-pyrrolo[2,3-d]pyrimidin-6-yl)phenyl)-4-((3-(2-(piperazin-1-ylmethyl)acrylamido)piperidin-1-yl)methyl)picolinamide trihydrochloride (25 mg, 0.0323 mmol) was added. After 20 minutes the reaction was diluted with ethyl acetate (20 mL). The mixture was washed with 5% lithium chloride (2×10 mL), saturated sodium bicarbonate (2×10 mL) and saturated ammonium chloride (10 mL). The organic phase was dried over sodium sulfate and the solvent was removed under reduced pressure. The residue was subjected to preparative HPLC(5-85% acetonitrile/water, 0.1% trifluoroacetic acid). The fractions containing product were combined and subjected to lyophilization, providing (R)-4-((3-(2-((4-(acetylglycyl)piperazin-1-yl)methyl)acrylamido)piperidin-1-yl)methyl)-N-(4-(4-morpholino-7H-pyrrolo[2,3-d]pyrimidin-6-yl)phenyl)picolinamide trifluoroacetate (Compound X)(6.6 mg, 0.0071 mmol, 22%).

LCMS=[M+H]⁺: 764.50; Purity=95%

¹H NMR (400 MHz, MeOD) δ 8.75 (d, J=4.9 Hz, 1H), 8.31 (s, 1H), 8.22 (s, 1H), 7.87 (d, J=8.4 Hz, 2H), 7.81 (s, OH), 7.67 (d, J=5.0 Hz, 1H), 7.20 (s, 1H), 6.23 (s, 1H), 5.96 (s, 1H), 4.43 (m, 2H), 4.12 (m, 1H), 4.00 (t, J=4.9 Hz, 4H), 3.95 (s, 2H), 3.88 (s, 2H), 3.83 (t, J=4.8 Hz, 4H), 3.79-3.56 (m, 4H), 3.43 (m, 1H), 3.35 (m, 1H), 3.50-3.19 (m, 6H), 2.09-1.93 (m, 1H), 1.90 (s, 3H), 1.83 (m, 1H), 1.62 (m, 1H).

### Example 135

### Compound 1067

### Preparation of 1-(4-(5-(4-(4-morpholino-7H-pyrrolo[2,3-d]pyrimidin-6-yl) phenoxy)pyrimidin-2-yl)piperazin-1-yl)prop-2-en-1-one (Compound 1067)

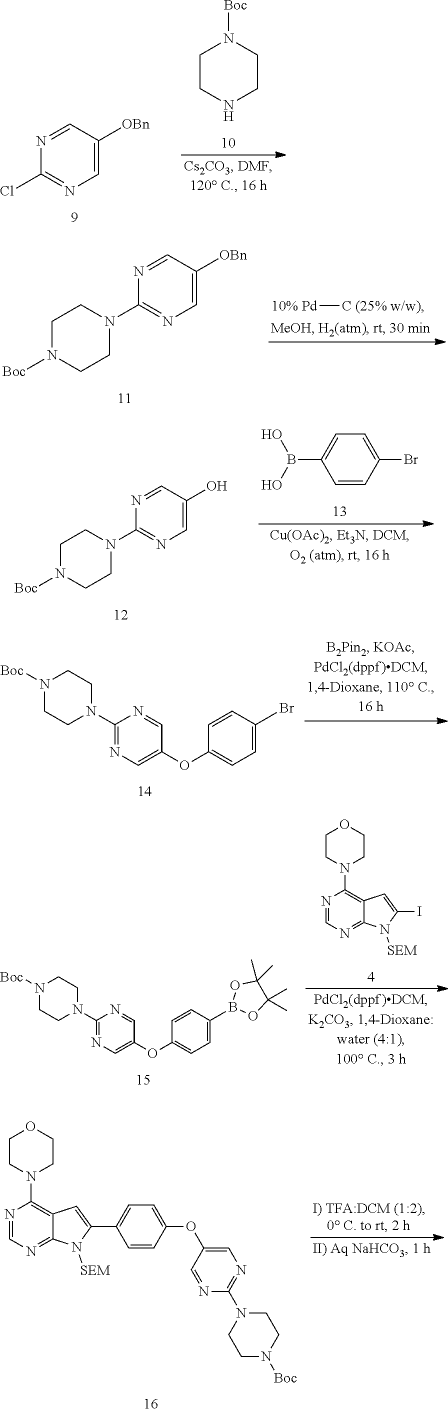

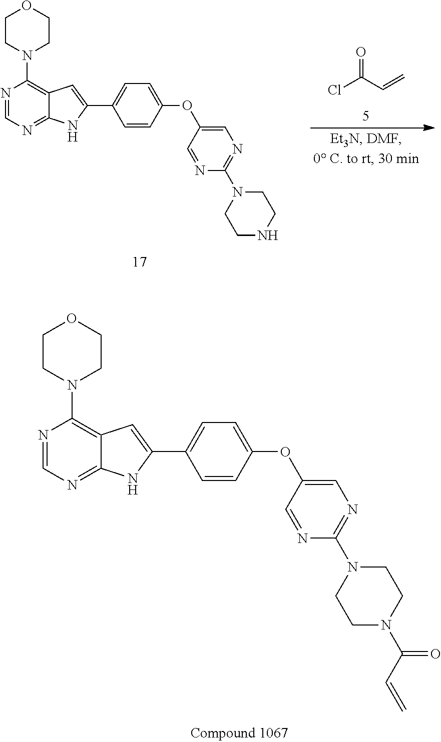

### Synthesis of tert-butyl 4-(5-(benzyloxy)pyrimidin-2-yl)piperazine-1-carboxylate (11)

To a stirred solution of 5-(benzyloxy)-2-chloropyrimidine 9 (2 g, 9.06 mmol) and tert-butyl piperazine-1-carboxylate 10 (2.53 g, 13.6 mmol) in DMF (20 mL) was added Cs₂CO₃ (8.86 g, 27.2 mmol) at RT. The resulting reaction mixture was stirred at 120° C. for 16 h. After completion of reaction (monitored by TLC), the reaction mixture was poured into ice-cold water (100 mL) and extracted with EtOAc (3×100 mL). The combined organic layer was washed with ice-cold brine solution (2×80 mL), dried over Na₂SO₄, filtered and evaporated under reduced pressure. The Crude was purified by flash chromatography (silica gel 230-400 M, 24 g SNAP) using eluents 40-50% EtOAc in heptane to get the title compound as a white solid 11 (1.4 g, 39%).

LCMS=[M+H]⁺: 371.36; Purity=99.7%.

### Synthesis of tert-butyl 4-(5-hydroxypyrimidin-2-yl)piperazine-1-carboxylate (12)

To a stirred solution of tert-butyl 4-(5-(benzyloxy)pyrimidin-2-yl)piperazine-1-carboxylate 11 (500 mg, 1.35 mmol) in MeOH (10 mL) was added 10% Pd—C(130 mg) at RT under H₂ atmosphere. The resulting reaction mixture was stirred at RT for 30 min. After completion of reaction (monitored by TLC), the reaction mixture was passed through celite bed and washed with MeOH (100 mL). The filtrate was concentrated under reduced pressure to get the title compound as a white solid 12 (350 mg, 90%), which was directly used in the next step without any purification.

LCMS=[M−H]: 279.19; Purity=95%.

### Synthesis of tert-butyl 4-(5-(4-bromophenoxy)pyrimidin-2-yl)piperazine-1-carboxylate (14)

To a stirred solution of tert-butyl 4-(5-hydroxypyrimidin-2-yl)piperazine-1-carboxylate 12 (300 mg, 1.07 mmol) and (4-bromophenyl) boronic acid 13 (322 mg, 1.61 mmol) in DCM (10 mL) was added Et₃N (0.5 mL, 3.21 mmol) at RT. The reaction mixture was purged with O2 for 15 min, followed by Cu (OAc) 2 (292 mg, 1.61 mmol) was added to this reaction mixture. The resulting reaction mixture was stirred at RT for 3 h. After the completion of reaction (monitored by TLC and LCMS), the reaction mass was filter through the celite bed was washed with DCM (100 mL). The filtrate was concentrated evaporated under reduced pressure. The crude was purified by flash chromatography (silica gel 230-400 M, 12 g SNAP) using eluents 45-50% EtOAc in heptane to get the title compound as white solid 14 (250 mg, 54%).

LCMS=[M+H]⁺: 437.21; Purity=86%.

### Synthesis of tert-butyl 4-(5-(4-(4,4,5,5-tetramethyl-1,3,2-dioxaborolan-2-yl) phenoxy)pyrimidin-2-yl)piperazine-1-carboxylate (15)

To a stirred solution of tert-butyl 4-(5-(4-bromophenoxy)pyrimidin-2-yl)piperazine-1-carboxylate 14 (250 mg, 0.574 mmol) and B₂Pin₂ (175 mg, 0.689 mmol) in 1,4-dioxane (8 mL) was added KOAc (170 mg, 1.72 mmol). The resulting reaction mixture was degassed with N₂ gas for 10 min, followed by added PdCl₂(dppf)·DCM (47 mg, 0.057 mmol) to the reaction mixture and stirred at 110° C. for 16 h. After completion of reaction (monitored by TLC and LCMS), the reaction mixture was filtered through celite bed and washed with EtOAc (80 mL). The filtrate was evaporated under reduced pressure to get the title compound as brown sticky oil 15 (260 mg; crude), which was used in next step directly without further purification.

### Synthesis of tert-butyl 4-(5-(4-(4-morpholino-7-((2-(trimethylsilyl)ethoxy)methyl)-7H-pyrrolo[2,3-d]pyrimidin-6-yl) phenoxy)pyrimidin-2-yl)piperazine-1-carboxylate (16)

To a stirred solution of tert-butyl 4-(5-(4-(4,4,5,5-tetramethyl-1,3,2-dioxaborolan-2-yl) phenoxy)pyrimidin-2-yl)piperazine-1-carboxylate 15 (250 mg, 0.518 mmol) and 4-(6-iodo-7-((2-(trimethylsilyl)ethoxy)methyl)-7H-pyrrolo[2,3-d]pyrimidin-4-yl)morpholine 4 (159 mg, 0.346 mmol) in 1,4 -Dioxane (8 mL) and H₂O (2 mL) was added K₂CO₃ (143 mg, 1.04 mmol) at RT. The resulting reaction mixture was degassed with N₂ gas for 10 min, followed by PdCl₂(dppf)·DCM (28.2 mg, 34.6 μmol) was added to the reaction mixture and stirred at 100° C. for 3 h. After the completion of reaction (monitored by TLC and LCMS monitoring), the reaction mixture was diluted with the water (50 mL) and extracted with EtOAc (3×60 mL). The combined organic layer was dried over Na₂SO₄, filtered and concentrated under reduced pressure. The crude was purified by flash chromatography (silica gel 230-400 M, 12 g SNAP) using eluent 40-50% EtOAc in heptane to get the title compound as a brown solid 16 (190 mg, 80%).

LCMS=[M+H]⁺: 689.42; Purity=92%.

### Synthesis of 4-(6-(4-((2-(piperazin-1-yl)pyrimidin-5-yl)oxy)phenyl)-7H-pyrrolo[2,3-d]pyrimidin-4-yl)morpholine (17)

To an ice cold stirred solution of tert-butyl 4-(5-(4-(4-morpholino-7-((2-(trimethylsilyl)ethoxy)methyl)-7H-pyrrolo[2,3-d]pyrimidin-6-yl) phenoxy)pyrimidin-2-yl)piperazine-1-carboxylate 16 (190 mg, 0.276 mmol) in DCM (4 mL) was added TFA (2 mL) dropwise at 0° C. under N₂ atmosphere. The reaction mixture was stirred at RT for 2 h. After completion of reaction (monitored by TLC and LCMS), the reaction mixture was concentrated under reduced pressure. The obtained crude was dissolved in 25% IPA in CHCl₃ (6 mL) and followed by saturated aq. NaHCO₃ (6 mL) was added and stirred at RT for 1 h. After completion of reaction (monitored by LCMS), the reaction was poured into ice-cold water (50 mL) and extracted with 25% IPA in CHCl₃ (3×30 mL). The combined organic layer was dried over Na₂SO₄, filtered and concentrated under reduced pressure to get the title compound as a brown solid 17 (100 mg; crude), which was used in next step directly without further purification.

LCMS=[M+H]⁺: 459.24; Purity=80%. Synthesis of 1-(4-(5-(4-(4-morpholino-7H-pyrrolo[2,3-d]pyrimidin-6-yl) phenoxy)pyrimidin-2-yl)piperazin-1-yl)prop-2-en-1-one (Compound 1067)

To an ice cold stirred solution of 44-(6-(4-((2-(piperazin-1-yl)pyrimidin-5-yl)oxy)phenyl)-7H-pyrrolo[2,3-d]pyrimidin-4-yl)morpholine 17 (100 mg, 0.218 mmol) in DMF (4 mL) was added Et₃N (0.15 mL, 1.09 mmol) and stirred for 5 min. Then, a solution of acryloyl chloride 8 (22 mg, 0.240 mmol) in DMF (0.5 mL) was added dropwise to the reaction mixture at same temperature under N₂ atmosphere. The reaction mixture was stirred at RT for 1 h. After completion of reaction (monitored by TLC), the reaction mixture was poured into ice-cold water (50 mL), obtained precipitate was filtered through sintered funnel, washed with Et20 and dried under reduced pressure. The crude residue was purified by RP-HPLC [2-98% 0.1% TFA in Water/ACN; Column: SunFire C18 (19*250) 10μ] to get Compound 1067 (10 mg, 7%).

¹H NMR (400 MHz, DMSO-d₆): δ 12.61 (s, 1H), 8.40 (s, 2H), 8.26 (s, 1H), 7.90 (d, J=8.4 Hz, 2H), 8.23 (s, 1H), 7.07 (d, J=8.8 Hz, 2H), 6.86 (dd, J=16.8 Hz, 10.4 Hz, 1H), 6.15 (dd, J=16.4 Hz, 2.4 Hz, 1H), 5.72 (dd, J=10.4 Hz, 2.4 Hz, 1H), 3.92-3.90 (m, 4H), 3.83-3.76 (m, 8H), 3.69-3.65 (m, 4H).

LCMS=[M+H]⁺: 513.31; Purity=97.38%

### Example 136

### Compound 1068

### Preparation of (R)-4-((3-(2-((4-(methylsulfonyl)piperazin-1-yl)methyl)acrylamido)piperidin-1-yl)methyl)-N-(4-(4-morpholino-7H-pyrrolo[2,3-d]pyrimidin-6-yl)phenyl)picolinamide (Compound 1068) Step 1:2-((4-(methylsulfonyl)piperazin-1-yl)methyl)acrylic acid

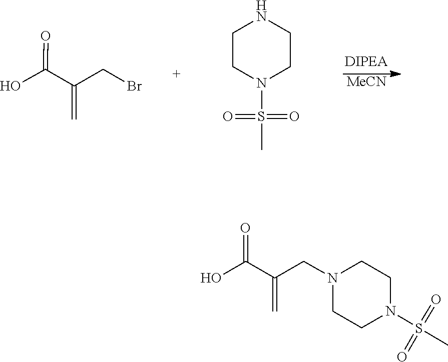

A solution of 2-(bromomethyl)prop-2-enoic acid (158 mg, 0.957 mmol), 1-(methylsulfonyl)piperazine (204.4 mg, 1.24 mmol), and N,N-diisopropylcthylamine (0.50 mL, 2.87 mmol) in acetonitrile (4 mL) was stirred at 35° C. for 18 hours, providing a solution of 2-((4-(methylsulfonyl)piperazin-1-yl)methyl)acrylic acid (237 mg, 0.954 mmol, 99%) in acetonitrile. The solution was used in the next step without workup.

LCMS=[M+H]⁺: 249.10

Step 2: (R)-4-((3-(2-((4-(methylsulfonyl)piperazin-1-yl)methyl)acrylamido)piperidin-1-yl)methyl)-N-(4-(4-morpholino-7H-pyrrolo[2,3-d]pyrimidin-6-yl)phenyl)picolinamide (Compound 1068)

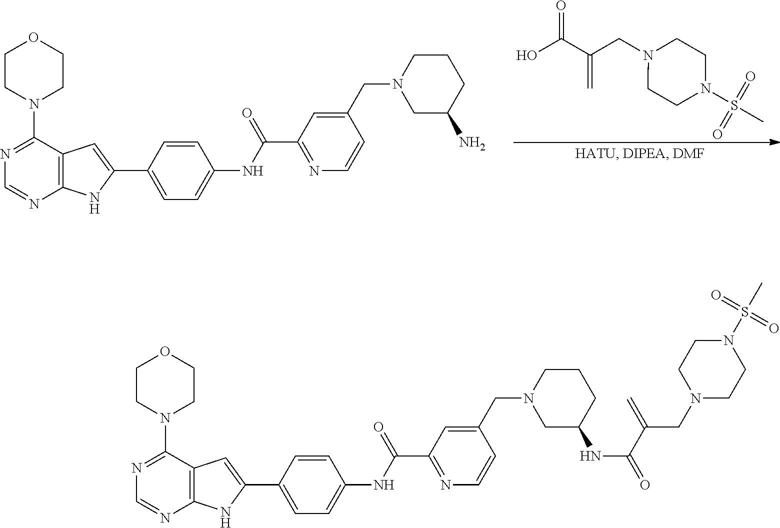 [SMILES: N[C@@H]1CCCN(Cc2ccnc(C(=O)Nc3ccc(-c4cc5c(N6CCOCC6)ncnc5[nH]4)cc3)c2)C1 ; C=C(CN1CCN(S(C)(=O)=O)CC1)C(=O)O ; C=C(CN1CCN(S(C)(=O)=O)CC1)C(=O)N[C@@H]1CCCN(Cc2ccnc(C(=O)Nc3ccc(-c4cc5c(N6CCOCC6)ncnc5[nH]4)cc3)c2)C1]

A solution of 2-((4-(methylsulfonyl)piperazin-1-yl)methyl)acrylic acid (0.23 mL, 0.0482 mmol)(0.21 M, reaction solution without workup) was added to a solution of O-(7-azabenzotriazol-1-yl)-N,N,N′,N′-tetramethyluronium hexafluorophosphate (20 mg, 0.0523 mmol) and N,N-diisopropylethylamine (0.042 mL, 0.241 mmol) in N,N-dimethylformamide (2 mL). After 5 minutes (R)-4-((3-aminopiperidin-1-yl)methyl)-N-(4-(4-morpholino-7H-pyrrolo[2,3-d]pyrimidin-6-yl)phenyl)picolinamide trihydrochloride (25 mg, 0.0402 mmol) was added. After 20 minutes the reaction was diluted with ethyl acetate (30 mL) and washed with 5% lithium chloride (2×10 mL), saturated sodium bicarbonate (2×10 mL) and saturated ammonium chloride (10 mL). The organic phase was dried over sodium sulfate and the solvent was removed under reduced pressure. The residue was subjected to preparative HPLC(5-85% acetonitrile/water, 0.1% trifluoroacetic acid). The fractions containing product were combined and subjected to lyophilization providing, (R)-4-((3-(2-((4-(methylsulfonyl) piperazin-1-yl)methyl)acrylamido)piperidin-1-yl)methyl)-N-(4-(4-morpholino-7H-pyrrolo[ 2,3-d]pyrimidin-6-yl)phenyl)picolinamide trifluoroacetate (Compound 1068)(14.8 mg, 0.0164 mmol, 40% yield)

LCMS=[M+H]⁺: 743.40; Purity=95%

¹H NMR (400 MHz, MeOD) δ 8.74 (d, J=4.9 Hz, 1H), 8.30 (s, 1H), 8.21 (d, J=1.8 Hz, 1H), 7.90-7.83 (m, 2H), 7.77 (dd, J=8.7, 1.9 Hz, 2H), 7.67 (d, J=5.0 Hz, 1H), 7.18 (d, J=1.9 Hz, 1H), 6.24 (s, 1H), 5.97 (s, 1H), 4.44 (s, 2H), 4.12 (m, 1H), 4.00 (t, J=4.9 Hz, 4H), 3.91 (s, 2H), 3.83 (t, J=4.4 Hz, 4H), 3.35-3.52 (m, 5H), 3.24 (m, 5H), 2.90-3.15 (m, 2H), 2.82 (d, J=1.7 Hz, 3H), 1.98 (m, 2H), 1.83 (m, 1H), 1.63 (m, 1H).

### Example 137

### Compound 1069

### Preparation of N-(4-(4-morpholino-7H-pyrrolo[2,3-d]pyrimidin-6-yl)phenyl)-4-(((3R)-3-(2-((3-(trifluoromethyl)pyrrolidin-1-yl)methyl)acrylamido)piperidin-1-yl)methyl)picolinamide (Compound 1069) Step 1:2-((3-(trifluoromethyl)pyrrolidin-1-yl)methyl)acrylic acid

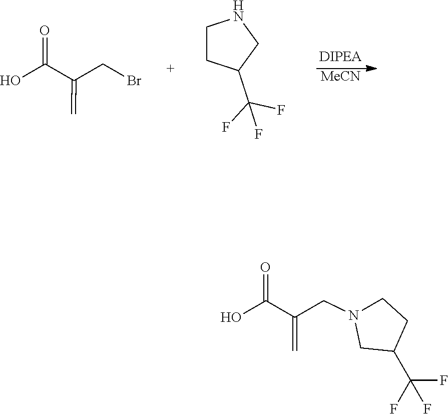

A solution of 2-(bromomethyl)prop-2-enoic acid (150 mg, 0.909 mmol), 3-(trifluoromethyl)pyrrolidine hydrochloride (207.5 mg, 1.18 mmol), and N,N-diisopropylethylamine (0.63 mL, 3.63 mmol) in acetonitrile (4 mL) was stirred at 35° C. for 18 hours, providing a solution of 2-((3-(trifluoromethyl)pyrrolidin-1-yl)methyl)acrylic acid (202 mg, 0.905 mmol, 99%) in acetonitrile. The solution was used in the next step without workup.

LCMS=[M−H]−: 222.10.

Step 2: N-(4-(4-morpholino-7H-pyrrolo[2,3-d]pyrimidin-6-yl)phenyl)-4-(((3R)-3-(2-((3-(trifluoromethyl)pyrrolidin-1-yl)methyl)acrylamido)piperidin-1-yl)methyl)picolinamide (Compound 1069)

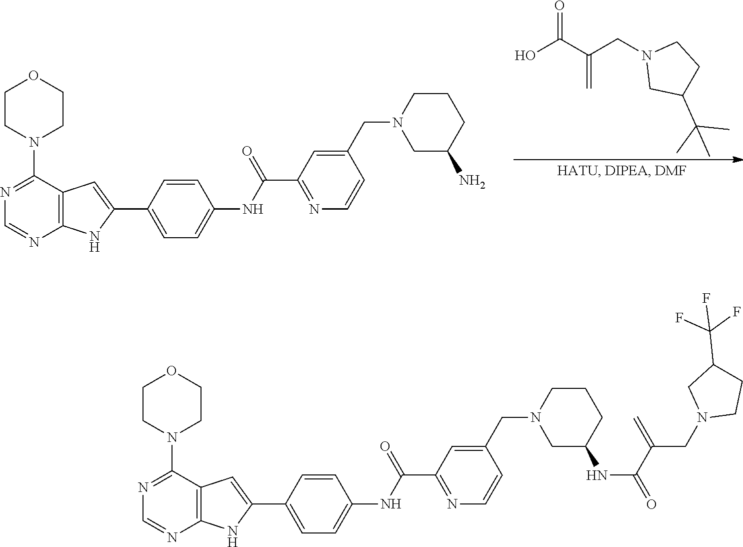 [SMILES: N[C@@H]1CCCN(Cc2ccnc(C(=O)Nc3ccc(-c4cc5c(N6CCOCC6)ncnc5[nH]4)cc3)c2)C1 ; C=C(CN1CCC(C(C)(C)C)C1)C(=O)O ; C=C(CN1CCC(C(F)(F)F)C1)C(=O)N[C@@H]1CCCN(Cc2ccnc(C(=O)Nc3ccc(-c4cc5c(N6CCOCC6)ncnc5[nH]4)cc3)c2)C1]

A solution of 2-((3-(trifluoromethyl)pyrrolidin-1-yl)methyl)acrylic acid (0.25 mL, 0.0482 mmol) (0.19 M, reaction solution without workup) was added to a solution of O-(7-azabenzotriazol-1-yl)-N,N,N′,N′ -tetramethyluronium hexafluorophosphate (20 mg, 0.0523 mmol) and N,N-diisopropylethylamine (0.042 mL, 0.241 mmol) in N,N-dimethylformamide (2 mL). After 5 minutes (R)-4-((3-aminopiperidin-1-yl)methyl)-N-(4-(4-morpholino-7H-pyrrolo[2,3-d]pyrimidin-6-yl)phenyl)picolinamide trihydrochloride (25 mg, 0.0402 mmol) was added. After 20 minutes the reaction was diluted with ethyl acetate (30 mL) and washed with 5% lithium chloride (2×10 mL), saturated sodium bicarbonate (2×10 mL) and saturated ammonium chloride (10 mL). The organic phase was dried over sodium sulfate and the solvent was removed under reduced pressure. The residue was subjected to preparative HPLC(5-85% acetonitrile/water, 0.1% trifluoroacetic acid). The fractions containing product were combined and subjected to lyophilization providing, N-(4-(4-morpholino-7H-pyrrolo[2,3-d]pyrimidin-6-yl)phenyl)-4-(((3R)-3-(2-((3-(trifluoromethyl)pyrrolidin-1-yl)methyl)acrylamido)piperidin-1-yl)methyl)picolinamide trifluoroacetate (Compound 1069)(6.3 mg, 0.0071 mmol, 17%).

LCMS=[M+H]⁺: 718.40; Purity=94%

¹H NMR (400 MHz, MeOD) δ 8.74 (d, J=5.0 Hz, 1H), 8.29 (s, 1H), 8.21 (s, 1H), 7.87 (d, J=8.3 Hz, 2H), 7.79 (d, J=8.3 Hz, 2H), 7.67 (d, J=4.9 Hz, 1H), 7.18 (s, 1H), 6.19 (s, 1H), 5.98 (s, 1H), 4.40 (s, 2H), 4.12 (m, 1H), 3.98 (m, 6H), 3.83 (m, 4H), 3.62 (m, 1H), 3.25-3.45 (m, 6H), 3.10-2.81 (m, 2H), 2.30 (dd, J=14.5, 7.5 Hz, 1H), 2.13 (dd, J=14.2, 7.1 Hz, 1H), 1.97 (t, J=17.4 Hz, 2H), 1.87-1.77 (m, 1H), 1.60 (m, 1H). 19F NMR (377 MHz, MeOD) δ-72.70.

### Example 138

### Compound 1070

### Preparation of 4-(((R)-3-(2-((4-(acetyl-D-valyl)piperazin-1-yl)methyl)acrylamido)piperidin-1-yl)methyl)-N-(4-(4-morpholino-7H-pyrrolo[2,3-d]pyrimidin-6-yl)phenyl)picolinamide (Compound 1070)

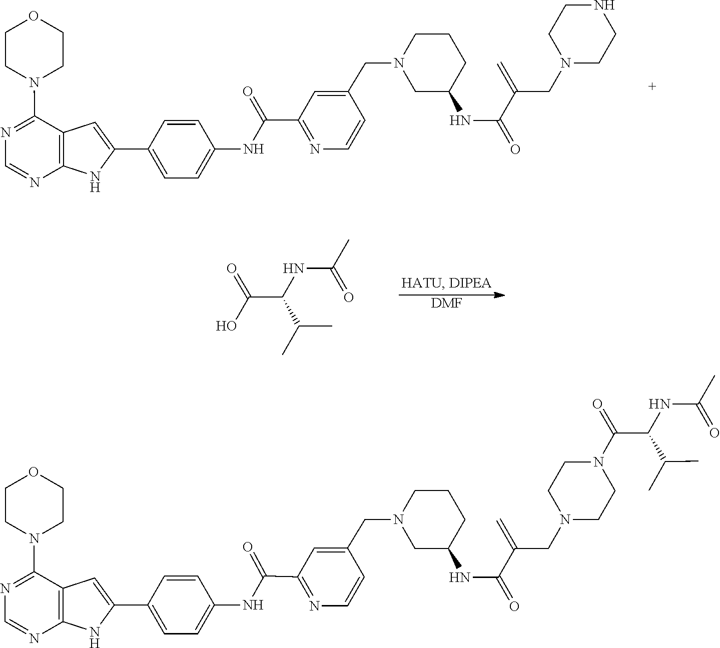

N,N-Diisopropylethylamine (0.045 mL, 0.258 mmol) was added to a solution of O-(7-azabenzotriazol-1-yl)-N,N,N′,N′-tetramethyluronium hexafluorophosphate (16 mg, 0.0420 mmol) and acetyl-D-valine (6.1 mg, 0.0388 mmol) in N,N-dimethylformamide (1 mL). After 5 minutes (R)—N-(4-(4-morpholino-7H-pyrrolo[2,3-d]pyrimidin-6-yl)phenyl)-4-((3-(2-(piperazin-1-ylmethyl)acrylamido)piperidin-1-yl)methyl)picolinamide trihydrochloride (25 mg, 0.0323 mmol) was added. After 45 minutes the reaction was diluted with ethyl acetate (20 mL). The mixture was washed with 5% lithium chloride (2×10 mL), saturated sodium bicarbonate (2×10 mL) and saturated ammonium chloride (10 mL). The organic phase was dried over sodium sulfate and the solvent was removed under reduced pressure. The residue was subjected to preparative HPLC(5-85% acetonitrile/water, 0.1% trifluoroacetic acid). The fractions containing product were combined and subjected to lyophilization, providing 4-(((R)-3-(2-((4-(acetyl-D-valyl)piperazin-1-yl)methyl)acrylamido)piperidin-1-yl)methyl)-N-(4-(4-morpholino-7H-pyrrolo[2,3-d]pyrimidin-6-yl)phenyl)picolinamide (Compound 1070)(8.4 mg, 0.0087 mmol, 26%).

LCMS=[M+H]⁺: 806.50; Purity=94%

¹H NMR (400 MHz, MeOD) δ 8.75 (d, J=4.9 Hz, 1H), 8.30 (s, 1H), 8.21 (s, 1H), 7.88 (d, J=8.3 Hz, 2H), 7.79 (d, J=8.4 Hz, 2H), 7.68 (d, J=5.0 Hz, 1H), 7.19 (s, 1H), 6.23 (s, 1H), 5.96 (s, 1H), 4.44 (m, 3H), 4.12 (s, 2H), 4.00 (t, J=4.9 Hz, 5H), 3.89 (d, J=4.3 Hz, 2H), 3.83 (t, J=4.9 Hz, 5H), 3.67 (m, 1H), 3.26-3.55 (m, 2H), 3.14 (m, 4H), 2.96 (m, 4H), 2.08-1.91 (m, 2H), 1.88 (s, 3H), 1.86-1.72 (m, 1H), 1.62 (m, 1H), 0.87 (d, J=6.7 Hz, 3H), 0.82 (d, J=6.7 Hz, 3H).

### Example 139

### Compound 1071

### Preparation of 4-(((R)-3-(2-((4-(acetyl-D-alanyl)piperazin-1-yl)methyl)acrylamido)piperidin-1-yl)methyl)-N-(4-(4-morpholino-7H-pyrrolo[2,3-d]pyrimidin-6-yl)phenyl)picolinamide (Compound 1071)

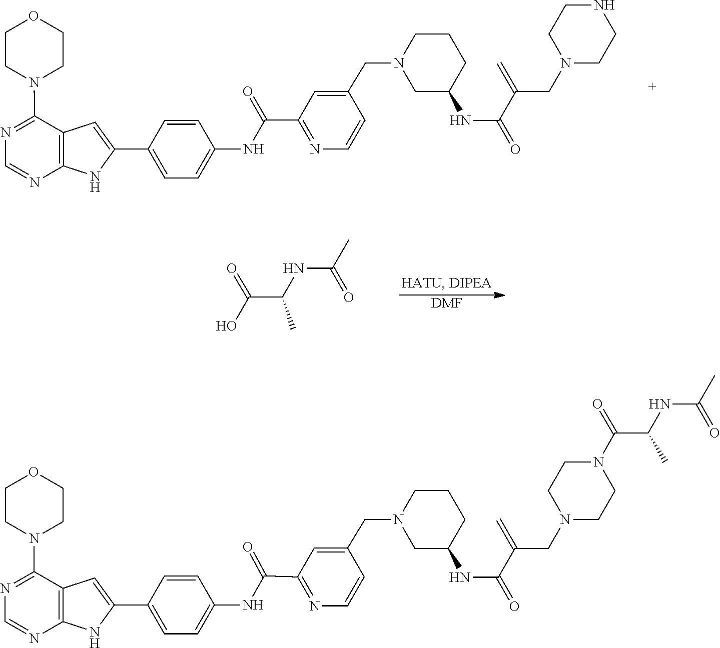

N,N-Diisopropylethylamine (0.045 mL, 0.258 mmol) was added to a solution of O-(7-azabenzotriazol-1-yl)-N,N,N′.N′-tetramethyluronium hexafluorophosphate (16 mg, 0.0420 mmol) and acetyl-D-alanine (6.1 mg, 0.0388 mmol) in N,N-dimethylformamide (1 mL). After 5 minutes (R)—N-(4-(4-morpholino-7H-pyrrolo[2,3-d]pyrimidin-6-yl)phenyl)-4-((3-(2-(piperazin-1-ylmethyl)acrylamido)piperidin-1-yl)methyl)picolinamide trihydrochloride (25 mg, 0.0323 mmol) was added. After 20 minutes the reaction was diluted with ethyl acetate (20 mL). The mixture was washed with 5% lithium chloride (2×10 mL), saturated sodium bicarbonate (2×10 mL) and saturated ammonium chloride (10 mL). The organic phase was dried over sodium sulfate and the solvent was removed under reduced pressure. The residue was subjected to preparative HPLC(5-85% acetonitrile/water, 0.1% trifluoroacetic acid). The fractions containing product were combined and subjected to lyophilization, providing 4-(((R)-3-(2-((4-(acetyl-D-alanyl)piperazin-1-yl)methyl)acrylamido)piperidin-1-yl)methyl)-N-(4-(4-morpholino-7H-pyrrolo[2,3-d]pyrimidin-6-yl)phenyl)picolinamide trifluoroacetate (Compound 1071)(7.5 mg, 0.0080 mmol, 24%).

LCMS=[M+H]⁺: 778.50; Purity=95%.

¹H NMR (400 MHz, MeOD) δ 8.75 (d, J=4.9 Hz, 1H), 8.30 (s, 1H), 8.21 (s, 1H), 7.88 (d, J=8.4 Hz, 2H), 7.79 (d, J=8.3 Hz, 2H), 7.67 (d, J=5.0 Hz, 1H), 7.19 (s, 1H), 6.23 (s, 1H), 5.95 (s, 1H), 4.66 (d, J=7.7 Hz, 2H), 4.42 (s, 2H), 4.12 (m, 1H), 4.00 (t, J=4.9 Hz, 4H), 3.88 (m, 2H), 3.83 (m, 5H), 3.70 (m, 2H), 3.54- 3.29 (m, 2H), 3.15 (m 2H), 2.85-3.10 (m, 4H), 1.97 (m, 1H), 1.85 (s, 3H), 1.82 (m, 1H), 1.62 (m, 1H), 1.18 (d, J=7.0 Hz, 3H).

### Example 140

### Compound 1072 and Compound 1085

### Preparation of (R)-1-(4-(5-(hydroxy (4-(4-morpholino-7H-pyrrolo[2,3-d]pyrimidin-6-yl)phenyl)methyl)pyrimidin-2-yl)piperazin-1-yl)prop-2-en-1-one (Compound 1072) and(S)-1-(4-(5-(hydroxy (4-(4-morpholino-7H-pyrrolo[2,3-d]pyrimidin-6-yl)phenyl)methyl)pyrimidin-2-yl)piperazin-1-yl)prop-2-en-1-one (Compound 1085)

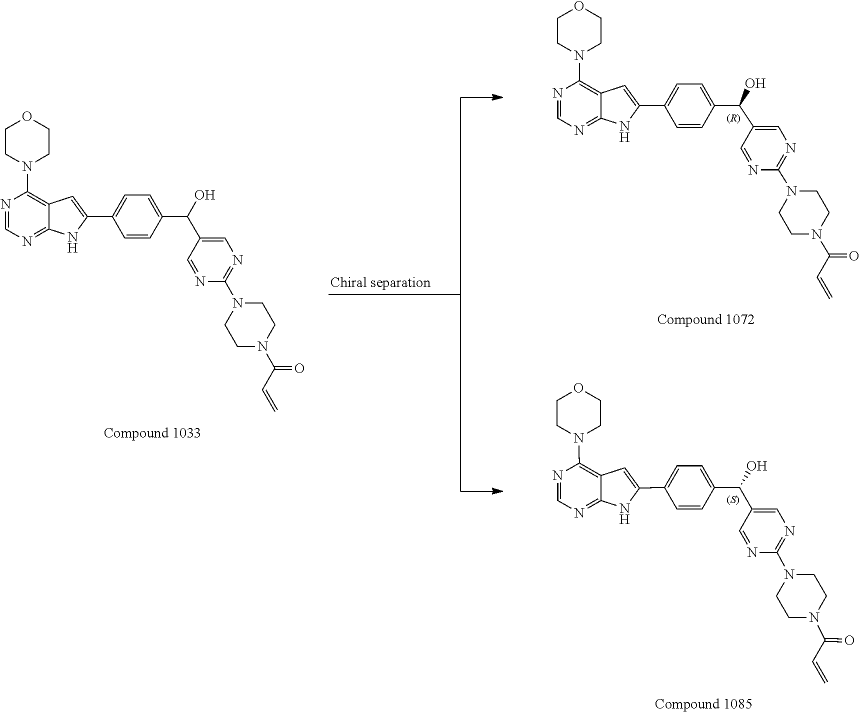

Chiral separation of Compound 1033 resulted in both enantiomers (R)-1-(4-(5-(hydroxy (4-(4-morpholino-7H-pyrrolo[2,3-d]pyrimidin-6-yl)phenyl)methyl)pyrimidin-2-yl)piperazin-1-yl)prop-2-en-1-one (Compound 1072) and(S)-1-(4-(5-(hydroxy (4-(4-morpholino-7H-pyrrolo[2,3-d]pyrimidin-6-yl)phenyl)methyl)pyrimidin-2-yl)piperazin-1-yl)prop-2-en-1-one (Compound 1085)

Note: Chirality arbitrarily assigned

Method for Chiral HPLC purification: Column: CHIRALPAK-IE (21×250 mm; 5u) Mobile Phase: A) 0.1% TEA in MeOH; B) DCM; Flow rate: Isocratic 24 mL/Min, wavelength: 250 nm & 330 nm

Analytical data of Compound 1072:

¹H NMR (400 MHz, DMSO-d₆): δ 12.33 (s, 1H), 8.35 (s, 2H), 8.20 (s, 1H), 7.87 (d, J=8.0 Hz, 2H), 7.44 (d, J=8.4 Hz, 2H), 7.19 (s, 1H), 6.82 (dd, J=16.8 Hz, 10.4 Hz, 1H), 6.13 (dd, J=16.8 Hz, 2.0 Hz, 1H), 5.97 (brs, 1H), 5.71-5.67 (m, 2H), 3.90-3.88 (m, 4H), 3.75-3.72 (m, 8H), 3.62-3.59 (m, 4H).

LCMS=[M+H]⁺: 527.33; Purity=96.11% and ee=99%

Analytical data of 1085:

¹H NMR (400 MHz, DMSO-d₆): δ 12.26 (s, 1H), 8.35 (s, 2H), 8.19 (s, 1H), 7.87 (d, J=8.4 Hz, 2H), 7.43 (d, J=8.4 Hz, 2H), 7.17 (s, 1H), 6.82 (dd, J=16.7 Hz, 10.4 Hz, 1H), 6.13 (dd, J=16.8 Hz, 2.2 Hz, 1H), 5.97 (brs, 1H), 5.71-5.67 (m, 2H), 3.89-3.87 (m, 4H), 3.76-3.73 (m, 8H), 3.62-3.59 (m, 4H).

LCMS=[M+H]⁺: 527.33; Purity=96.98% and ee=98%

### Example 141

### Compound 1074

### Preparation of 2-(azetidin-1-ylmethyl)-N-((3R)-1-(5-(hydroxy (4-(4-morpholino-7H-pyrrolo[2,3-d]pyrimidin-6-yl)phenyl)methyl)pyrimidin-2-yl)piperidin-3-yl)acrylamide (Compound 1074)

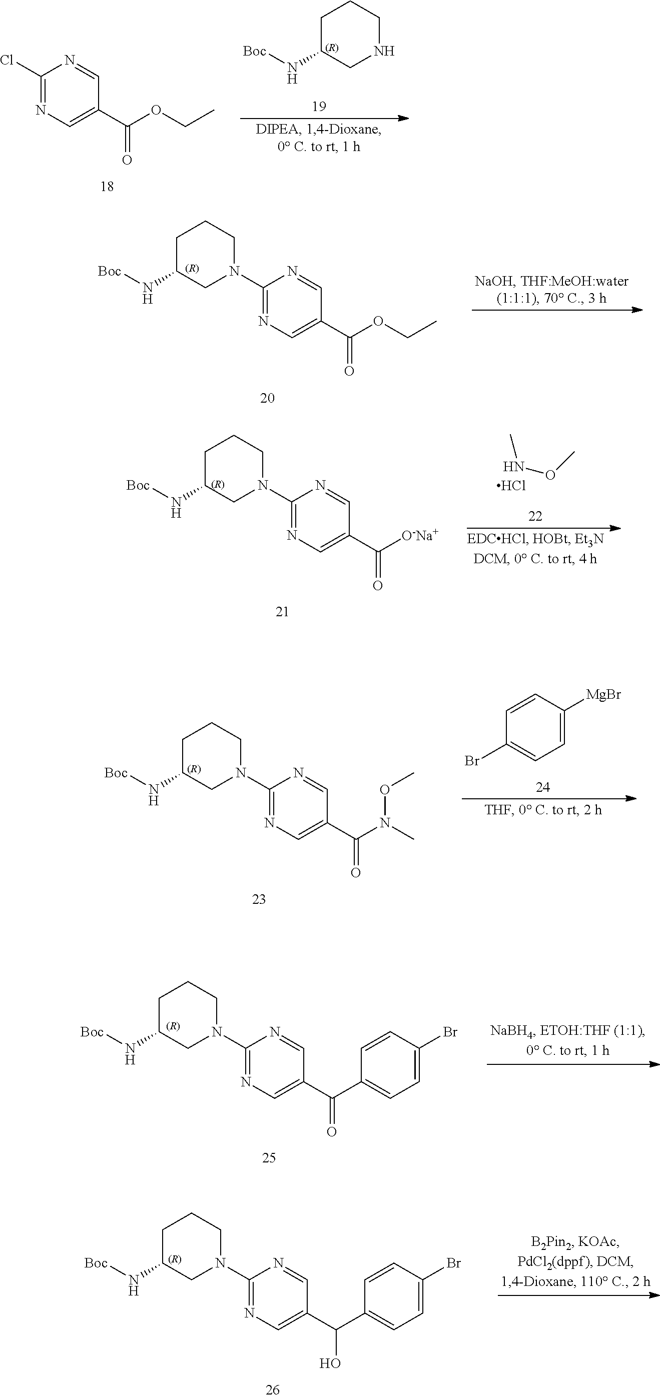

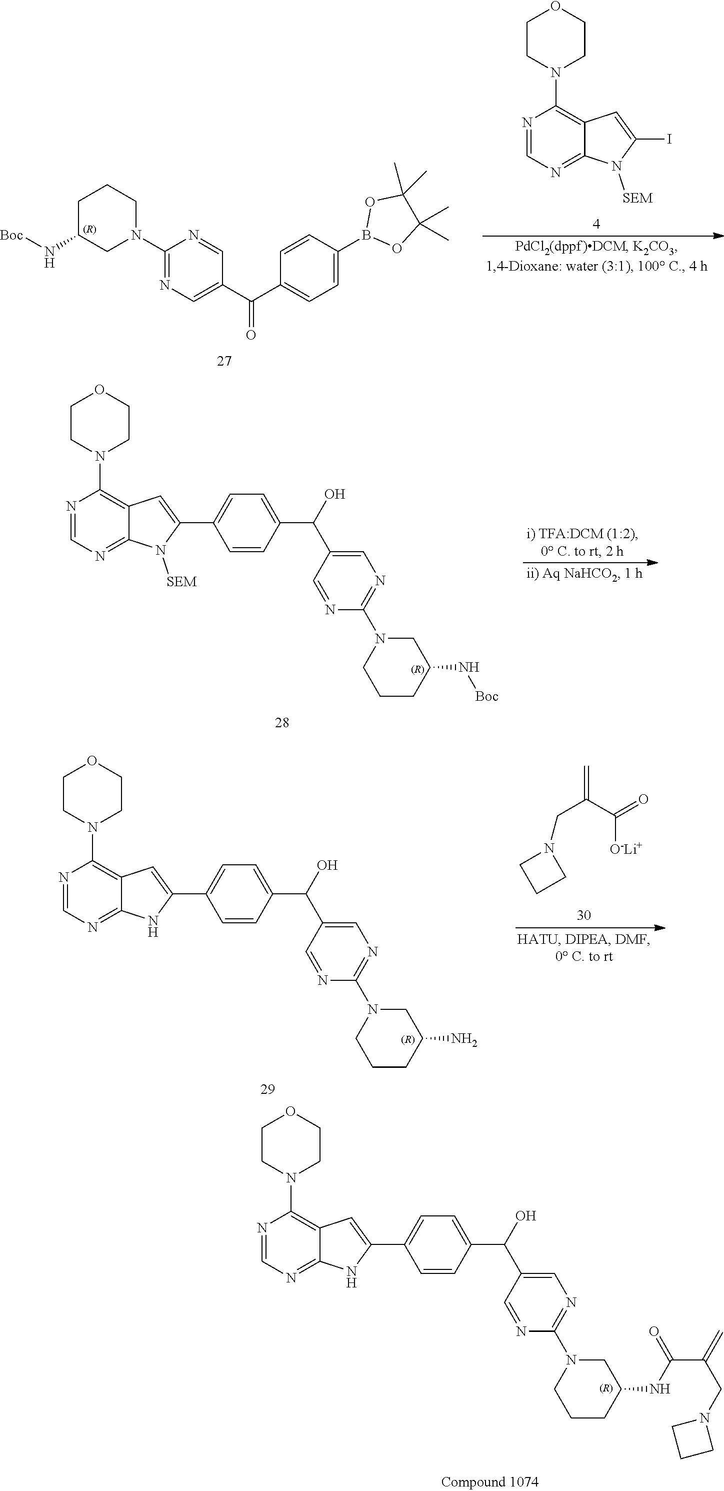

### Synthesis of ethyl (R)-2-(3-((tert-butoxycarbonyl)amino)piperidin-1-yl)pyrimidine-5-carboxylate (20)

To an ice cold stirred solution of ethyl 2-chloropyrimidine-5-carboxylate 18 (5 g, 26.08 mmol) and tert-butyl (R)-piperidin-3-ylcarbamate 19 (6.44, 32.2 mmol) in 1,4-Dioxane (50 mL) was added DIPEA (10.2 mL, 59 mmol) at RT and stirred for 1 h. After completion of reaction (monitored by TLC), the reaction mixture was concentrated under reduced pressure. The crude was purified by flash chromatography (silica gel 230-400 M, 80 g SNAP) using eluents 25-30% EtOAc in heptane to get the title compound as a white solid 20 (8 g, 85%).

LCMS=[M+H]⁺: 351.43; Purity=95%.

### Synthesis of sodium (R)-2-(3-((tert-butoxycarbonyl)amino)piperidin-1-yl)pyrimidine-5-carboxylate (21)

To a stirred solution of ethyl (R)-2-(3-((tert-butoxycarbonyl)amino)piperidin-1-yl)pyrimidine-5-carboxylate 20 (8 g, 22.8 mmol) in THF (30 mL) and MeOH (30 mL) was added aqueous solution of NaOH (1.83 g, 45.7 mmol) dissolved in H₂O (30 mL) at RT. The reaction mixture was stirred at 70° C. for 3 h. After completion of reaction (monitored by TLC), the reaction mixture was concentrated under reduced pressure. The crude was dissolved in 25% IPA in CHCl₃ (100 mL), stirred for 10 min and filtered through sintered funnel. The filtrate was concentrated under reduced pressure to get the title compound as a white solid 21 (7 g, 93%).

LCMS=[M−H]: 321.30; Purity=98.16%.

### Synthesis of tert-butyl (R)-(1-(5-(methoxy(methyl)carbamoyl)pyrimidin-2-yl)piperidin-3-yl)carbamate (23)

To an ice cold stirred solution of sodium (R)-2-(3-((tert-butoxycarbonyl)amino)piperidin-1-yl)pyrimidine-5-carboxylate 21 (5 g, 15.5 mmol) in DCM (50 mL) were added Et₃N (8.7 mL, 62 mmol), HOBt (2.52 g, 18.6 mmol) and EDC·HCl (4.46 g, 23.3 mmol) sequentially to this reaction under N₂ atmosphere. The reaction mixture was stirred at RT for 1 h. Then, N,O-dimethylhydroxylamine hydrochloride 22 (1.82 g, 18.6 mmol) was added to the reaction mixture and stirred at RT for 4 h. After completion of reaction (monitored by TLC and LCMS), the reaction mixture was poured into water (100 mL) and extracted with DCM (2×100 mL). The combined organic layer was washed with brine solution (2×100 mL), dried over Na₂SO₄, filtered and evaporated under reduced pressure. The crude was purified over the flash chromatography (silica gel 230-400 M, 80 g SNAP) using eluents 30-40% EtOAc in heptane to get the title compound as a white solid 23 (1.8 g, 32%).

LCMS=[M+H]⁺: 366.35; Purity=98.52%.

### Synthesis of tert-butyl (R)-(1-(5-(4-bromobenzoyl)pyrimidin-2-yl)piperidin-3-yl)carbamate (25)

To an ice cold stirred solution of tert-butyl (R)-(1-(5-(methoxy(methyl)carbamoyl)pyrimidin-2-yl)piperidin-3-yl)carbamate 23 (1.6 g, 4.38 mmol) and dry THF (20 mL) was added (4-bromophenyl) magnesium bromide 24 (35 mL, 17.5 mmol; 0.5 M in THF) dropwise to the reaction mixture under N₂ atmosphere. The reaction mixture was stirred at RT for 2 h. After completion of reaction (monitored by TLC and LCMS), the reaction mixture was quenched with 1N HCl (60 mL) and extracted with EtOAc (3×80 mL). The combined organic layer was dried over Na₂SO₄, filtered and concentrated under reduced pressure. The crude was purified by flash column chromatography (silica gel 230-400 M, 24 g SNAP) using eluents 10-12% EtOAc in heptane to get the title compound as a white solid 25 (1.1 g, 54%).

LCMS=[M+H]⁺: 461.19; Purity=82%.

### Synthesis of tert-butyl ((3R)-1-(5-((4-bromophenyl)(hydroxy)methyl)pyrimidin-2-yl)piperidin-3-yl)carbamate (26)

To an ice cold stirred solution of tert-butyl (R)-(1-(5-(4-bromobenzoyl)pyrimidin-2-yl)piperidin-3-yl)carbamate 25 (1.1 g, 2.38 mmol) in EtOH (10 mL) and THF (10 mL) was added NaBH₄ (180 mg, 4.77 mmol) at same temperature under N₂ atmosphere. The reaction mixture was stirred at RT for 1 h. After completion of reaction (monitored by TLC), the reaction mixture was concentrated under reduced pressure. The crude residue was diluted with water (50 mL) and extracted with DCM (3×80 mL). The combined organic layer was washed with brine solution (2×80 mL), dried over Na₂SO₄, filtered and evaporated under reduced pressure. The crude was purified by flash column chromatography (silica gel 230-400 M, 24 g SNAP) using eluents 25-30% EtOAc in heptane to get the title compound as white solid 26 (1 g, 90%).

LCMS=[M+H]⁺: 463.16; Purity=86%.

### Synthesis of tert-butyl ((3R)-1-(5-(hydroxy (4-(4,4,5,5-tetramethyl-1,3,2-dioxaborolan-2-yl)phenyl)methyl)pyrimidin-2-yl)piperidin-3-yl)carbamate (27)

To a stirred solution of tert-butyl ((3R)-1-(5-((4-bromophenyl)(hydroxy)methyl)pyrimidin-2-yl)piperidin-3-yl)carbamate 26 (1 g, 2.16 mmol) and B₂Pin₂ (822 mg, 3.24 mmol) in 1,4-dioxane (15 mL) was added KOAc (635 mg, 6.47 mmol) at RT. The resulting reaction mixture was degassed with N₂ gas for 10 min, followed by added PdCl₂(dppf)·DCM (176 mg, 0.216 mmol) to the reaction mixture and stirred at 100° C. for 2 h. After completion of reaction (monitored by TLC and LCMS), the reaction mixture was filtered through celite bed and washed with EtOAc (100 mL). The filtrate was evaporated under reduced pressure to get the title compound as brown sticky oil 27 (1 g; crude), which was used in next step directly without further purification.

### Synthesis of tert-butyl ((3R)-1-(5-(hydroxy (4-(4-morpholino-7-((2-(trimethylsilyl)ethoxy)methyl)-7H-pyrrolo[2,3-d]pyrimidin-6-yl)phenyl)methyl)pyrimidin-2-yl)piperidin-3-yl)carbamate (28)

To a stirred solution of tert-butyl ((3R)-1-(5-(hydroxy (4-(4,4,5,5-tetramethyl-1,3,2-dioxaborolan-2-yl)phenyl)methyl)pyrimidin-2-yl)piperidin-3-yl)carbamate 27 (1 g, 2.09 mmol) and 4-(6-iodo-7-((2-(trimethylsilyl)ethoxy)methyl)-7H-pyrrolo[2,3-d]pyrimidin-4-yl)morpholine 4 (800 mg, 1.74 mmol) in 1,4-dioxane (20 mL) and water (5 mL) was added K₂CO₃ (720 mg, 5.21 mmol) at RT. The resulting reaction mixture was degassed with argon gas for 15 min. Then was added PdCl₂(dppf)·DCM (142 mg, 0.174 mmol) and the resulting reaction mixture was stirred at 100° C. for 4 h. After the completion of reaction (monitored by TLC), the reaction mixture was diluted with water (50 mL) and extracted with EtOAc (3×80 mL). The combined organic layer was washed with brine solution (2×50 mL), dried over Na₂SO₄, filtered and evaporated under reduced pressure. The crude was purified by flash column chromatography (silica gel 230-400 M, 24 g SNAP) using eluents 50-60% EtOAc in heptane to get the title compound as a brown solid 28 (700 mg, 56%).

LCMS=[M+H]⁺: 717.54; Purity=85%.

### Synthesis of (4-(4-morpholino-7H-pyrrolo[2,3-d]pyrimidin-6-yl)phenyl)(2-(piperazin-1-yl)pyrimidin-5-yl)methanol (29)

To an ice cold stirred solution of tert-butyl ((3R)-1-(5-(hydroxy (4-(4-morpholino-7-((2-(trimethylsilyl)ethoxy)methyl)-7H-pyrrolo[2,3-d]pyrimidin-6-yl)phenyl)methyl)pyrimidin-2-yl)piperidin-3-yl)carbamate 28 (600 mg, 0.837 mmol) in DCM (6 mL) was added TFA (3 mL) dropwise under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 25% IPA in CHCl₃ (10 mL) and saturated NaHCO₃ (10 mL) was added and stirred at RT for 1 h. Then, the reaction mixture was extracted with 25% IPA in CHCl₃ (3×50 mL), dried over Na₂SO₄, filtered and concentrated under reduced pressure to get the title compound as brownish semi solid 29 (400 mg; crude), which was used in next step directly without further purification.

LCMS=[M+H]⁺: 487.36; Purity=83%.

### Synthesis of N,N-diethyl-4-(4-(5-(hydroxy (4-(4-morpholino-7H-pyrrolo[2,3-d]pyrimidin-6-yl)phenyl)methyl)pyrimidin-2-yl)piperazin-1-yl)but-2-ynamide (Compound 1074)

To an ice cold stirred solution of (4-(4-morpholino-7H-pyrrolo[2,3-d]pyrimidin-6-yl)phenyl)(2-(piperazin-1-yl)pyrimidin-5-yl)methanol 29 (400 mg, 0.822 mmol) and lithium 2-(azetidin-1-ylmethyl)acrylate 30 (335 mg, 2.47 mmol) in DMF (6 mL) were added DIPEA (0.45 mL, 2.47 mmol) and HATU (470 mg, 1.23 mmol) sequentially to the reaction mixture under N₂ atmosphere. The resulting reaction mixture was stirred at RT for 2 h. After completion of reaction (monitored by TLC and LCMS), the reaction mixture was quenched with ice-cold water (50 mL) and extracted with 25% IPA in CHCl₃ (3×50 mL). The combined organic layer was dried over Na₂SO₄, filtered and concentrated under reduced pressure. The crude was purified by RP-HPLC [5-95% 5 mM Ammonium Bicarbonate in water/ACN; Column: X Bridge C18 (19*250) 10μ] to get Compound 1074 (55 mg, 11%).

¹H NMR (400 MHz, DMSO-d₆): δ 12.22 (s, 1H), 9.05-9.02 (m, 1H), 8.29 (s, 2H), 8.17 (s, 1H), 7.86 (d, J=8.4 Hz, 2H), 7.40 (d, J=8.4 Hz, 2H), 7.16 (s, 1H), 5.93 (d, J=4.0 Hz, 1H), 5.75 (t, J=2.0 Hz, 1H), 5.63 (d, J=4.0 Hz, 1H), 5.32 (brs, 1H), 3.89-3.80 (m, 7H), 3.76-3.65 (m, 6H), 3.01 (q, J=13.2 Hz, 2H), 2.89 (q, J=7.2 Hz, 2H), 2.81 (q, J=6.4 Hz, 2H), 1.90-1.79 (m, 1H), 1.77-1.71 (m, 2H), 1.69-1.59 (m, 2H), 1.53-1.48 (m, 1H).

LCMS=[M+H]⁺: 610.46; Purity=95.99%

### Example 142

### Compound 1076

### Preparation of (R)-4-((3-(2-((2-oxa-6-azaspiro[3.3]heptan-6-yl)methyl)acrylamido)piperidin-1-yl)methyl)-N-(4-(4-morpholino-7H-pyrrolo[2,3-d]pyrimidin-6-yl)phenyl)picolinamide (Compound 1076)

Step 1:2-((2-oxa-6-azaspiro[3.3]heptan-6-yl)methyl)acrylic acid

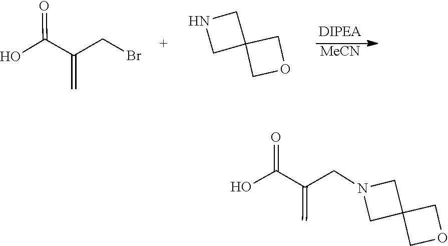

A solution of 2-(bromomethyl)prop-2-enoic acid (100 mg, 0.606 mmol), 2-oxa-6-azaspiro[3.3]heptane (78 mg, 0.788 mmol), and N,N-diisopropylethylamine (0.32 mL, 1.81 mmol) in acetonitrile (4 mL) was stirred at 35° C. for 18 hours, providing a solution of 2-((2-oxa-6-azaspiro[3.3]heptan-6-yl)methyl)acrylic acid (111 mg, 0.606 mmol, 99%) in acetonitrile. The solution was used in the next step without workup.

LCMS=[M−H]−: 182.10.

Step 2: (R)-4-((3-(2-((2-oxa-6-azaspiro[3.3]heptan-6-yl)methyl)acrylamido)piperidin-1-yl)methyl)-N-(4-(4-morpholino-7H-pyrrolo[2,3-d]pyrimidin-6-yl)phenyl)picolinamide (Compound 1076)

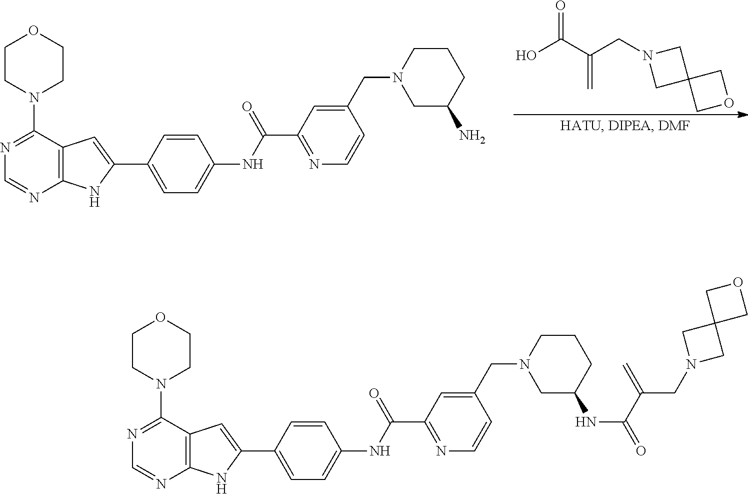 [SMILES: N[C@@H]1CCCN(Cc2ccnc(C(=O)Nc3ccc(-c4cc5c(N6CCOCC6)ncnc5[nH]4)cc3)c2)C1 ; C=C(CN1CC2(COC2)C1)C(=O)O ; C=C(CN1CC2(COC2)C1)C(=O)N[C@@H]1CCCN(Cc2ccnc(C(=O)Nc3ccc(-c4cc5c(N6CCOCC6)ncnc5[nH]4)cc3)c2)C1]

A solution of 2-((2-oxa-6-azaspiro[3.3]heptan-6-yl)methyl)acrylic acid (0.34 mL, 0.0482 mmol)(0.21 M, reaction solution without workup) was added to a solution of O-(7-azabenzotriazol-1-yl)-N,N,N′,N′-tetramethyluronium hexafluorophosphate (20 mg, 0.0523 mmol) and N,N-diisopropylethylamine (0.042 mL, 0.241 mmol) in N,N-dimethylformamide (2 mL). After 5 minutes (R)-4-((3-aminopiperidin-1-yl)methyl)-N-(4-(4-morpholino-7H-pyrrolo[2,3-d]pyrimidin-6-yl)phenyl)picolinamide trihydrochloride (25 mg, 0.0402 mmol) was added. After 20 minutes the reaction was diluted with ethyl acetate (30 mL) and washed with 5% lithium chloride (2×10 mL), saturated sodium bicarbonate (2×10 mL), saturated ammonium chloride (10 mL). The organic phase was dried over sodium sulfate and the solvent was removed under reduced pressure. The residue was subjected to preparative HPLC(5-85% acetonitrile/water, 0.1% trifluoroacetic acid). The fractions containing product were combined and subjected to lyophilization providing, (R)-4-((3-(2-((2-oxa-6-azaspiro[3.3]heptan-6-yl)methyl)acrylamido)piperidin-1-yl)methyl)-N-(4-(4-morpholino-7H-pyrrolo[2,3-d]pyrimidin-6-yl)phenyl)picolinamide trifluoroacetate (Compound 1076)(10.0 mg, 0.0120 mmol, 29%).

LCMS=[M+H]⁺: 678.40; Purity=95%

¹H NMR (400 MHz, MeOD) § 8.74 (d, J=4.9 Hz, 1H), 8.29 (s, 1H), 8.21 (s, 1H), 7.87 (d, J=8.4 Hz, 2H), 7.78 (d, J=8.4 Hz, 2H), 7.67 (d, J=4.9 Hz, 1H), 7.18 (s, 1H), 6.13 (s, 1H), 5.90 (s, 1H), 4.66 (m, 4H), 4.41 (s, 2H), 4.22 (m, 4H), 4.10 (m, 1H), 4.00 (t, J=4.9 Hz, 4H), 3.86 (s, 2H), 3.83 (t, J=4.8 Hz, 4H), 3.40 (d, J=11.2 Hz, 1H), 3.34 (d, J=11.8 Hz, 1H), 3.12-2.72 (m, 2H), 1.96 (m, 2H), 1.87-1.73 (m, 1H), 1.59 (m, 1H).

### Example 143

### Compound 1078

### (R)-4-((3-(2-((6-acetyl-2,6-diazaspiro[3.4]octan-2-yl)methyl)acrylamido)piperidin-1-yl)methyl)-N-(4-(4-morpholino-7H-pyrrolo[2,3-d]pyrimidin-6-yl)phenyl)picolinamide

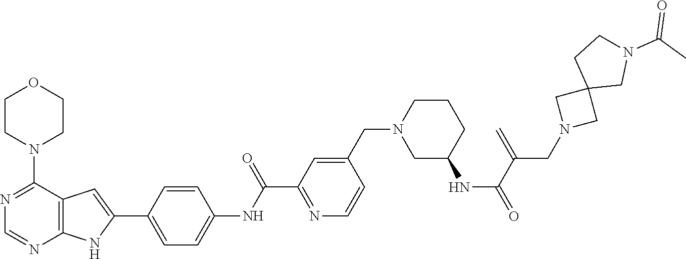 [SMILES: C=C(CN1CC2(CCN(C(C)=O)C2)C1)C(=O)N[C@@H]1CCCN(Cc2ccnc(C(=O)Nc3ccc(-c4cc5c(N6CCOCC6)ncnc5[nH]4)cc3)c2)C1]

Step 1:2-((6-(tert-butoxycarbonyl)-2,6-diazaspiro[3.4]octan-2-yl)methyl)acrylic acid

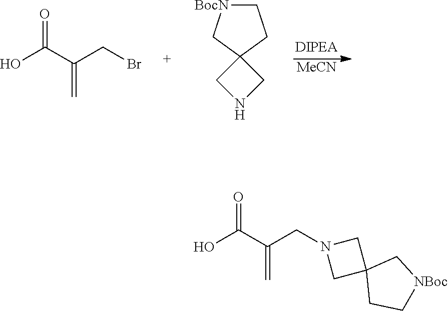

A solution of 2-(bromomethyl)prop-2-enoic acid (225 mg, 1.36 mmol), tert-butyl 2,6-diazaspiro[3.4]octane-6-carboxylate (376 mg, 1.77 mmol) and N,N-diisopropylethylamine (0.71 mL, 4.09 mmol) in acetonitrile (6 mL) was stirred at 35° C. for 16 hours, providing a solution of 2-((6-(tert-butoxycarbonyl)-2,6-diazaspiro[3.4]octan-2-yl)methyl)acrylic acid (404 mg, 1.36 mmol, 99%) in acetonitrile. The solution (0.2 M) was used in the next step without workup.

LCMS=[M+H]⁺: 297.20.

Step 2: tert-Butyl (R)-2-(2-((1-((2-((4-(4-morpholino-7H-pyrrolo[2,3-d]pyrimidin-6-yl)phenyl)carbamoyl)pyridin-4-yl)methyl)piperidin-3-yl)carbamoyl) allyl)-2,6-diazaspiro[3.4]octane-6-carboxylate

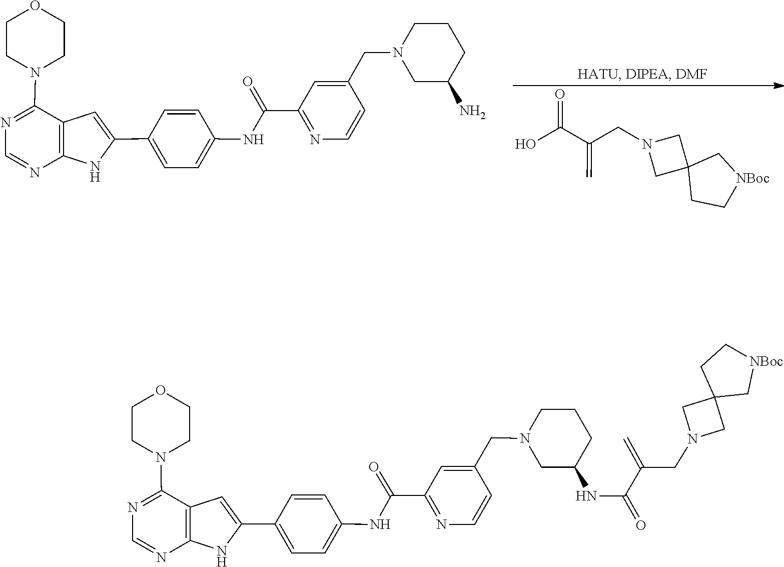 [SMILES: N[C@@H]1CCCN(Cc2ccnc(C(=O)Nc3ccc(-c4cc5c(N6CCOCC6)ncnc5[nH]4)cc3)c2)C1 ; C=C(CN1CC2(CCN(C(=O)OC(C)(C)C)C2)C1)C(=O)O ; C=C(CN1CC2(CCN(C(=O)OC(C)(C)C)C2)C1)C(=O)N[C@@H]1CCCN(Cc2ccnc(C(=O)Nc3ccc(-c4cc5c(N6CCOCC6)ncnc5[nH]4)cc3)c2)C1]

A solution of 2-((6-(tert-butoxycarbonyl)-2,6-diazaspiro[3.4]octan-2-yl)methyl)acrylic acid (0.87 mL, 0.192 mmol)(0.2 M, reaction mixture without workup) was added to a solution of O-(7-azabenzotriazol-1-yl)-N,N,N′.N′-tetramethyluronium hexafluorophosphate (79 mg, 0.209 mmol) and N,N-diisopropylethylamine (0.17 mL, 0.964 mmol) in N,N-dimethylformamide (2 mL). After 5 minutes (R)-4-((3-aminopiperidin-1-yl)methyl)-N-(4-(4-morpholino-7H-pyrrolo[2,3-d]pyrimidin-6-yl)phenyl)picolinamide trihydrochloride (100 mg, 0.161 mmol) was added. After 30 minutes the mixture was diluted with ethyl acetate (30 mL) and washed with 5% lithium chloride (2×10 mL), saturated sodium bicarbonate (2×10 mL), saturated ammonium chloride (10 mL). The organic phase was dried over sodium sulfate and the solvent was removed under reduced pressure. The residue was subjected to flash chromatography (0-100% 20% methanol in ethyl acetate/hexanes). The fractions containing product were combined and the solvent was removed under reduced pressure, providing tert-butyl (R)-2-(2-((1-((2-((4-(4-morpholino-7H-pyrrolo[2,3-d]pyrimidin-6-yl)phenyl)carbamoyl)pyridin-4-yl)methyl)piperidin-3-yl)carbamoyl) allyl)-2,6-diazaspiro[3.4]octane-6-carboxylate (71 mg, 0.0740 mmol, 46%).

LCMS=[M+H]⁺: 791.50.

Step 3: (R)-4-((3-(2-((2,6-diazaspiro[3.4]octan-2-yl)methyl)acrylamido)piperidin-1-yl)methyl)-N-(4-(4-morpholino-7H-pyrrolo[2,3-d]pyrimidin-6-yl)phenyl)picolinamide

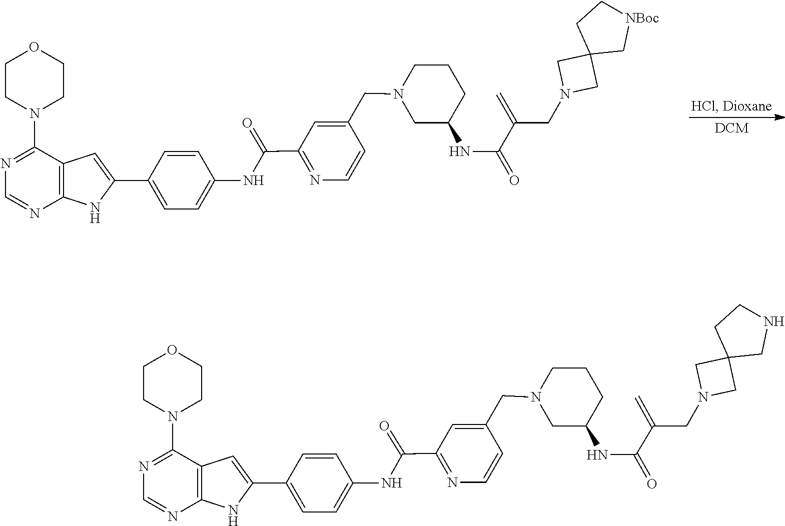 [SMILES: C=C(CN1CC2(CCN(C(=O)OC(C)(C)C)C2)C1)C(=O)N[C@@H]1CCCN(Cc2ccnc(C(=O)Nc3ccc(-c4cc5c(N6CCOCC6)ncnc5[nH]4)cc3)c2)C1 ; C=C(CN1CC2(CCNC2)C1)C(=O)N[C@@H]1CCCN(Cc2ccnc(C(=O)Nc3ccc(-c4cc5c(N6CCOCC6)ncnc5[nH]4)cc3)c2)C1]

A solution of hydrochloric acid in 1,4-dioxane (4N, 0.50 mL, 2.00 mmol) was added to a solution of tert-butyl (R)-2-(2-((1-((2-((4-(4-morpholino-7H-pyrrolo[2,3-d]pyrimidin-6-yl)phenyl)carbamoyl)pyridin-4-yl)methyl)piperidin-3-yl)carbamoyl) allyl)-2,6-diazaspiro[3.4]octane-6-carboxylate (71 mg, 0.0891 mmol) in dichloromethane (5 mL). After 20 minutes the volatiles were removed under reduced pressure, providing (R)-4-((3-(2-((2,6-diazaspiro[3.4]octan-2-yl)methyl)acrylamido)piperidin-1-yl)methyl)-N-(4-(4-morpholino-7H-pyrrolo[2,3-d]pyrimidin-6-yl)phenyl)picolinamide trihydrochloride (72 mg, 0.0089 mmol 99%).

LCMS=[M+H]⁺: 691.40.

Step 4: (R)-4-((3-(2-((6-acetyl-2,6-diazaspiro[3.4]octan-2-yl)methyl)acrylamido)piperidin-1-yl)methyl)-N-(4-(4-morpholino-7H-pyrrolo[2,3-d]pyrimidin-6-yl)phenyl)picolinamide (Compound 1078)

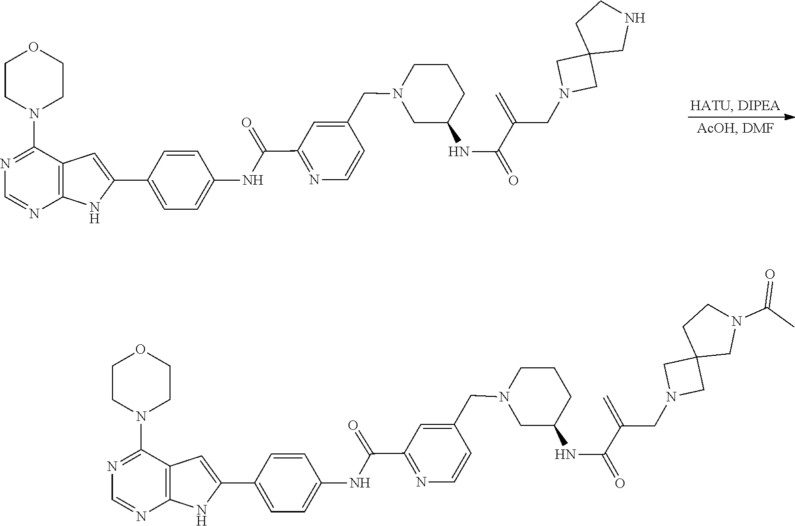 [SMILES: C=C(CN1CC2(CCNC2)C1)C(=O)N[C@@H]1CCCN(Cc2ccnc(C(=O)Nc3ccc(-c4cc5c(N6CCOCC6)ncnc5[nH]4)cc3)c2)C1 ; C=C(CN1CC2(CCN(C(C)=O)C2)C1)C(=O)N[C@@H]1CCCN(Cc2ccnc(C(=O)Nc3ccc(-c4cc5c(N6CCOCC6)ncnc5[nH]4)cc3)c2)C1]

N,N-Diisopropylethylamine (0.027 mL, 0.152 mmol) was added to a solution of O-(7-azabenzotriazol-1-yl)-N,N,N′.N′-tetramethyluronium hexafluorophosphate (9.4 mg, 0.0248 mmol) and acetic acid (1.3 mg, 0.0225 mmol) in N,N-dimethylformamide (1 mL). After 5 minutes (R)-4-((3-(2-((2,6 -diazaspiro[3.4]octan-2-yl)methyl)acrylamido)piperidin-1-yl)methyl)-N-(4-(4-morpholino-7H-pyrrolo[2,3-d]pyrimidin-6-yl)phenyl)picolinamide trihydrochloride (15.2 mg, 0.0191 mmol) was added. After 20 minutes the reaction was diluted with ethyl acetate (20 mL). The mixture was washed with 5% lithium chloride (3×10 mL) saturated sodium bicarbonate (2×10 mL) and ammonium chloride (10 mL). The organic phase was dried over sodium sulfate and the solvent was removed under reduced pressure. The residue was subjected to preparative HPLC(5-85% acetonitrile/water, 0.1% trifluoroacetic acid). The fractions containing product were combined and subjected to lyophilization providing, (R)-4-((3-(2-((6-acetyl-2,6-diazaspiro[3.4]octan-2-yl)methyl)acrylamido)piperidin-1-yl)methyl)-N-(4-(4-morpholino-7H-pyrrolo[2,3-d]pyrimidin-6-yl)phenyl)picolinamide trifluoroacetate (Compound 1078)(54.5 mg, 0.0052 mmol, 27%).

LCMS=[M+H]⁺: 733.40; Purity=95%

¹H NMR (400 MHz, MeOD) δ 8.74 (d, J=5.0 Hz, 1H), 8.30 (s, 1H), 8.21 (s, 1H), 7.88 (d, J=8.3 Hz, 2H), 7.79 (dd, J=8.8, 3.1 Hz, 2H), 7.67 (d, J=5.0 Hz, 1H), 7.20 (s, 1H), 6.16 (s, 1H), 5.95 (s, 1H), 4.42 (s, 2H), 4.10 (m, 2H), 4.06 (m, 3H), 3.99 (m, 6H), 3.83 (m, 4H), 3.64 (m, 1H), 3.55 (m, 1H), 3.42 (m, 2H), 3.31 (m, 2H), 3.14-2.80 (m, 2H), 2.20 (t, J=7.0 Hz, 1H), 2.10 (t, J=7.2 Hz, 1H), 2.00 (m, 1H), 1.90 (m, 3H), 1.81 (m, 1H), 1.62 (m, 1H).

### Example 144

### Compound 1082

### Preparation of (R)-2-((2-oxa-6-azaspiro[3.3]heptan-6-yl)methyl)-N-(1-(5-((4-(4-morpholino-7H-pyrrolo[2,3-d]pyrimidin-6-yl)phenyl)amino)pyrimidin-2-yl)piperidin-3-yl)acrylamide (Compound 1082)

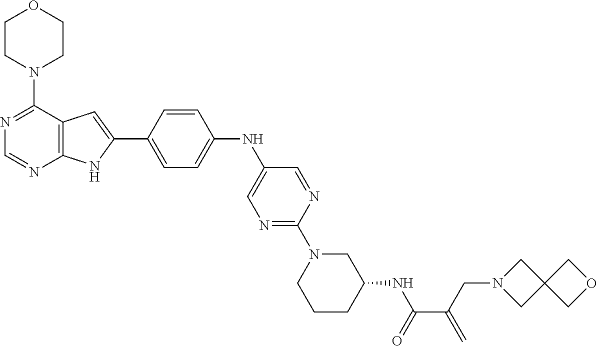 [SMILES: C=C(CN1CC2(COC2)C1)C(=O)N[C@@H]1CCCN(c2ncc(Nc3ccc(-c4cc5c(N6CCOCC6)ncnc5[nH]4)cc3)cn2)C1]

Step 1:2-((2-oxa-6-azaspiro[3.3]heptan-6-yl)methyl)acrylic acid

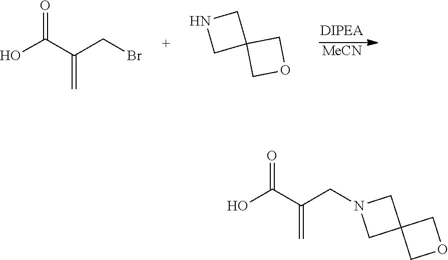

A solution of 2-(bromomethyl)prop-2-enoic acid (100 mg, 0.606 mmol), 2-oxa-6-azaspiro[3.3]heptane (78 mg, 0.788 mmol), and N,N-diisopropylethylamine (0.32 mL, 1.81 mmol) in acetonitrile (4 mL) was stirred at 35° C. for 18 hours, providing a solution of 2-((2-oxa-6-azaspiro[3.3]heptan-6-yl)methyl)acrylic acid (111 mg, 0.606 mmol, 99%) in acetonitrile. The solution was used in the next step without workup.

LCMS=[M−H]−: 182.10.

### tert-Butyl (R)-(1-(5-((4-(4-morpholino-7H-pyrrolo[2,3-d]pyrimidin-6-yl)phenyl)amino)pyrimidin-2-yl)piperidin-3-yl)carbamate

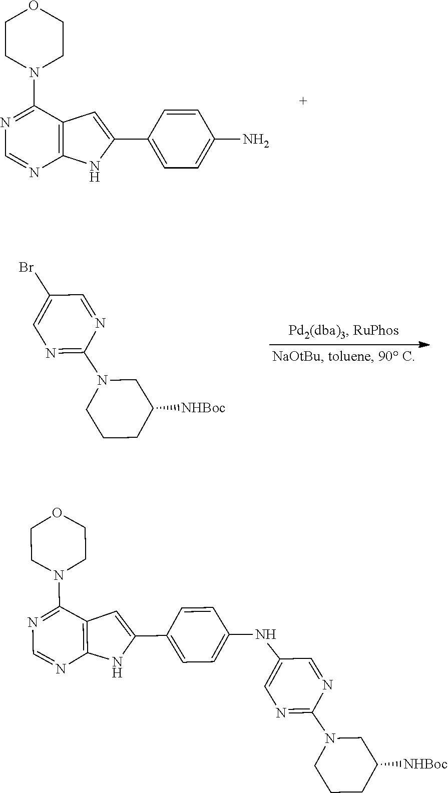 [SMILES: Nc1ccc(-c2cc3c(N4CCOCC4)ncnc3[nH]2)cc1 ; CC(C)(C)OC(=O)N[C@@H]1CCCN(c2ncc(Br)cn2)C1 ; CC(C)(C)OC(=O)N[C@@H]1CCCN(c2ncc(Nc3ccc(-c4cc5c(N6CCOCC6)ncnc5[nH]4)cc3)cn2)C1]

A mixture of 4-(4-morpholino-7H-pyrrolo[2,3-d]pyrimidin-6-yl)aniline (413 mg, 1.40 mmol), dicyclohexyl-[2-(2,4,6-triisopropylphenyl)phenyl]phosphane (67 mg, 0.140 mmol) and tert-butyl (R)-(1-(5-bromopyrimidin-2-yl)piperidin-3-yl)carbamate (500 mg, 1.40 mmol) in toluene (10 mL) was degassed for 2 minutes. Tris (dibenzylideneacetone) dipalladium (0)(128 mg, 0.140 mmol) was added and the Mixture was degassed for 2 minutes. sodium tert butoxide (202 mg, 2.10 mmol) was added and the mixture was degassed for 2 minutes. The was stirred at 90° C. for 4 hours. The mixture was partitioned with ethyl acetate (100 mL) and saturated ammonium chloride (50 mL). The solid which formed was removed by filtration. The organic phase was washed with saturated ammonium chloride (50 mL) and dried over sodium sulfate. The solvent was removed under reduced pressure. The residue was subjected to flash chromatography (0-100%, 20% methanol in ethyl acetate/hexanes). The fractions containing product were combined and the solvent was removed under reduced pressure. Fractions containing product were combined and the solvent was removed under reduced pressure. The residue was subjected to preparative HPLC(5-85% acetonitrile/water 0.1% trifluoroacetic acid). The fractions containing product were combined and subjected to lyophilization, providing tert-butyl (R)-(1-(5-((4-(4-morpholino-7H-pyrrolo[2,3-d]pyrimidin-6-yl)phenyl)amino)pyrimidin-2-yl)piperidin-3-yl) carbamate trifluoroacetate (190 mg, 0.278 mmol, 19%).

LCMS=[M+H]⁺: 572.40; Purity=95%

### (R)-2-(3-aminopiperidin-1-yl)-N-(4-(4-morpholino-7H-pyrrolo[2,3-d]pyrimidin-6-yl)phenyl)pyrimidin-5-amine

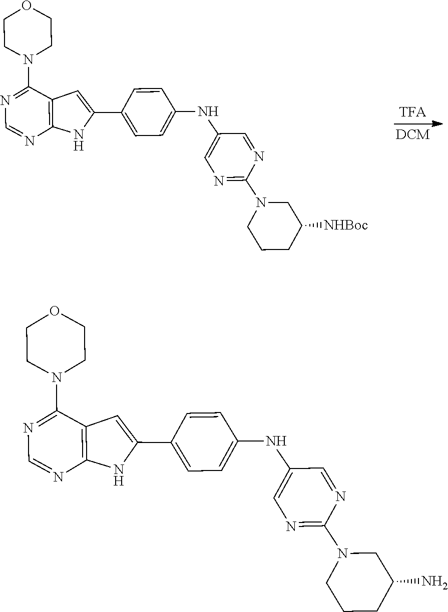 [SMILES: CC(C)(C)OC(=O)N[C@@H]1CCCN(c2ncc(Nc3ccc(-c4cc5c(N6CCOCC6)ncnc5[nH]4)cc3)cn2)C1 ; N[C@@H]1CCCN(c2ncc(Nc3ccc(-c4cc5c(N6CCOCC6)ncnc5[nH]4)cc3)cn2)C1]

### A solution of tert-butyl (R)-(1-(5-((4-(4-morpholino-7H-pyrrolo[2,3-d]pyrimidin-6-yl)phenyl)amino)pyrimidin-2-yl)piperidin-3-yl)carbamate

trifluoroacetate (190 mg, 0.278 mmol) and hydrochloric acid in 1,4-dioxane (4N, 2.1 mL, 8.33 mmol) in dichloromethane (5 mL) was stirred for 20 minutes. The volatiles were removed under reduced pressure. The residue was subjected to preparative HPLC(5-85%, acetonitrile/water, 0.1% trifluoroacetic acid). The fractions containing product were combined and subjected to lyophilization, providing (R)-2-(3-aminopiperidin-1-yl)-N-(4-(4-morpholino-7H-pyrrolo[2,3-d]pyrimidin-6-yl)phenyl)pyrimidin-5-amine trifluoroacetate (142 mg, 0.243 mmol, 87%).

LCMS=[M+H]⁺: 472.30; Purity=95%

Step 2: (R)-4-((3-(2-((2-oxa-6-azaspiro[3.3]heptan-6-yl)methyl)acrylamido)piperidin-1-yl)methyl)-N-(4-(4-morpholino-7H-pyrrolo[2,3-d]pyrimidin-6-yl)phenyl)picolinamide (Compound 1082)

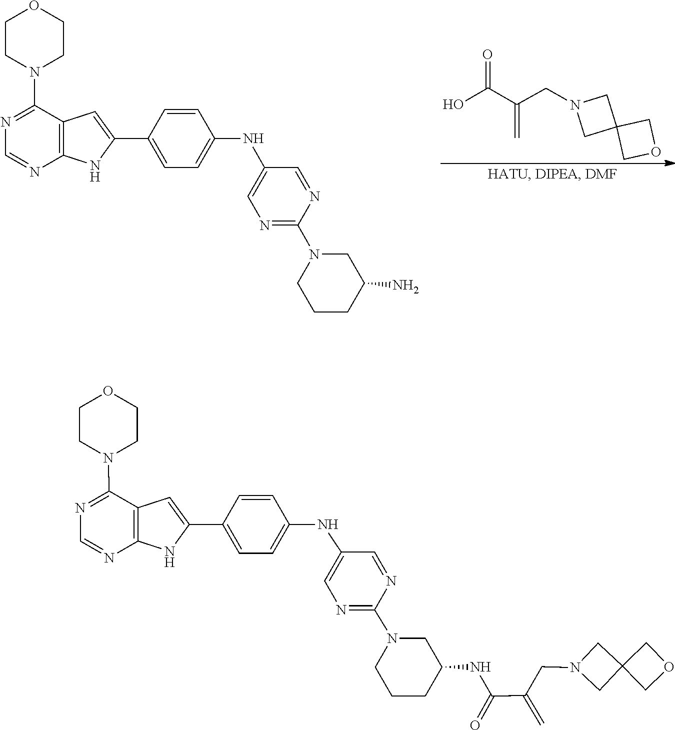 [SMILES: N[C@@H]1CCCN(c2ncc(Nc3ccc(-c4cc5c(N6CCOCC6)ncnc5[nH]4)cc3)cn2)C1 ; C=C(CN1CC2(COC2)C1)C(=O)O ; C=C(CN1CC2(COC2)C1)C(=O)N[C@@H]1CCCN(c2ncc(Nc3ccc(-c4cc5c(N6CCOCC6)ncnc5[nH]4)cc3)cn2)C1]

(R)-2-(3-Aminopiperidin-1-yl)-N-(4-(4-morpholino-7H-pyrrolo[2,3-d]pyrimidin-6-yl)phenyl)pyrimidin-5-amine trifluoroacetate (21 mg, 0.0359 mmol) was added to a solution of 2-((2-oxa-6-azaspiro[3.3]heptan-6-yl)methyl)acrylic acid (0.38 mL, 0.0538 mmol)(0.14 M, reaction solution without workup), O-(7-azabenzotriazol-1-yl)-N,N,N′,N′-tetramethyluronium hexafluorophosphate (20.4 mg, 0.0538 mmol) and N,N-diisopropylethylamine (0.050 mL, 0.287 mmol) in N,N-dimethylformamide (1 mL). After 30 minutes the reaction was diluted with ethyl acetate (25 mL) and washed with 5% lithium chloride (2×10 mL), saturated sodium bicarbonate (2×10 mL), and brine (10 mL). The organic phase was dried over sodium sulfate and the solvent was removed under reduced pressure. The residue was subjected to preparative HPLC(5-85% acetonitrile/water, 0.1% trifluoroacetic acid). The fractions containing product were combined and subjected to lyophilization providing, (R)-4-((3-(2-((2-oxa-6-azaspiro[3.3]heptan-6-yl)methyl)acrylamido)piperidin-1-yl)methyl)-N-(4-(4-morpholino-7H-pyrrolo[2,3-d]pyrimidin-6-yl)phenyl)picolinamide trifluoroacetate (Compound 1082)(7.0 mg, 0.0093 mmol, 26%).

LCMS=[M+H]⁺: 637.40; Purity=95%

¹H NMR (400 MHz, MeOD) § 8.18 (d, J=2.9 Hz, 3H), 7.60-7.52 (m, 2H), 7.00 (s, 1H), 6.85-6.75 (m, 2H), 6.08 (s, 1H), 5.87 (s, 1H), 4.69-4.60 (m, 4H), 4.50 (dd, J=12.6, 4.2 Hz, 1H), 4.36 (d, J=13.6 Hz, 1H), 4.15-4.32 (m, 4H), 3.97 (t, J=4.9 Hz, 4H), 3.93-3.85 (m, 2H), 3.82 (m, 5H), 3.06-2.97 (m, 1H), 2.93 (dd, J=12.7, 9.8 Hz, 1H), 1.94 (m, 1H), 1.77 (m, 1H), 1.54 (m, 2H).

### Example 145

### Compound 1083

Preparation 1-(3-(5-((4-(4-morpholino-7H-pyrrolo[2,3-d]pyrimidin-6-yl)phenyl)amino)pyrimidin-2-yl)-3,8-diazabicyclo[3.2.1]octan-8-yl)prop-2-en-1-one (Compound 1083)

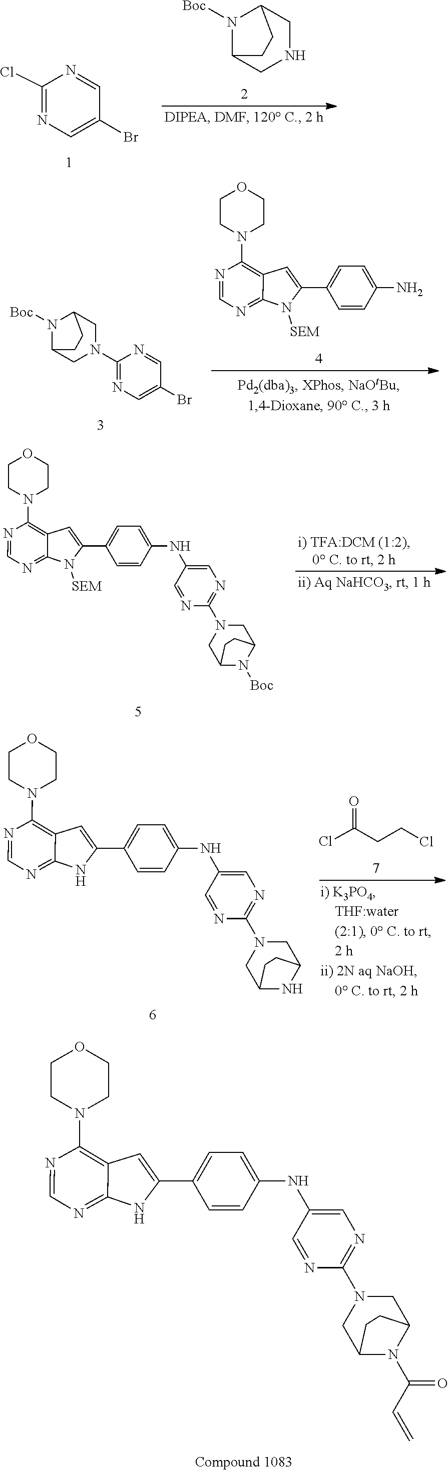

### Synthesis of tert-butyl 3-(5-bromopyrimidin-2-yl)-3,8-diazabicyclo[3.2.1]octane-8-carboxylate (3)

To an ice cold stirred solution of 5-bromo-2-chloropyrimidine 1 (2 g, 10.3 mmol) and tert-butyl 3,8-diazabicyclo[3.2.1]octane-8-carboxylate 2 (4.4 g, 20.7 mmol) in DMF (20 mL) was added DIPEA (5.3 mL, 31 mmol). The reaction mixture was stirred at 120° C. for 2 h. After completion of reaction (monitored by TLC and LCMS), the reaction mixture was diluted with ice water (80 mL) and extracted with EtOAc (3×80 mL). The combined organic layer was dried over Na₂SO₄, filtered and evaporated under reduced pressure. The crude was purified by flash chromatography (silica gel 230-400 M, 40 g SNAP) using eluents 30-40% EtOAc in heptane to get the title compound as light yellowish liquid 3 (3.8 g, 99%).

LCMS=[M+H]⁺: 369.01; Purity=99%.

### Synthesis of tert-butyl 3-(5-((4-(4-morpholino-7-((2-(trimethylsilyl)ethoxy)methyl)-7H-pyrrolo[2,3-d]pyrimidin-6-yl)phenyl)amino)pyrimidin-2-yl)-3,8-diazabicyclo[3.2.1]octane-8-carboxylate (5)

To a stirred solution of tert-butyl 3-(5-bromopyrimidin-2-yl)-3,8-diazabicyclo[3.2.1]octane-8-carboxylate 3 (1 g, 2.71 mmol) and 4-(4-morpholino-7-((2-(trimethylsilyl)ethoxy)methyl)-7H-pyrrolo[2,3-d]pyrimidin-6-yl)aniline 4 (1.73 g, 4.06 mmol) in 1,4-Dioxane (20 mL) was added NaO^{t}Bu (390 mg, 4.06 mmol) at RT. The resulting reaction mixture was degassed with N₂ gas for 10 min, followed by Pd₂(dba)₃ (248 mg, 0.271 mmol) and XPhos (129 mg, 0.271 mmol) were added and the reaction mixture was stirred at 90° C. for 3 h. After completion of reaction (monitored by TLC and LCMS), the reaction mixture was diluted with water (50 mL) and extracted with EtOAc (3×80 mL). The combined organic layer was dried over Na₂SO₄, filtered and evaporated under reduced pressure. The crude was purified by flash chromatography (silica gel 230-400 M, 40 g SNAP) using eluents 30-40% EtOAc in heptane to get the title compound as a yellow solid 5 (1.5 g, 77%).

LCMS=[M+H]⁺: 714.05; Purity=70%.

### Synthesis of 2-(3,8-diazabicyclo[3.2.1]octan-3-yl)-N-(4-(4-morpholino-7H-pyrrolo[2,3-d]pyrimidin-6-yl)phenyl)pyrimidin-5-amine (6)

To an ice cold stirred solution of tert-butyl 3-{5-[p-(4-morpholino-1-{[2-(trimethylsilyl) ethoxy]methyl}-1H-1,5,7-triazainden-2-yl)phenylamino]-2-pyrimidinyl}-3,8-diazabicyclo[3.2.1]octane-8-carboxylate 5 (1.5 g, 2.1 mmol) in DCM (16 mL) was added TFA (8 mL) dropwise under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 25% IPA in CHCl₃ (15 mL) and followed by saturated aq. NaHCO₃ (10 mL) was added and stirred at RT for 30 min. After completion of reaction (monitored by LCMS), the reaction was poured into ice-cold water (50 mL) and extracted with 25% IPA in CHCl₃ (3×70 mL). The combined organic layer was dried over Na₂SO₄, filtered and concentrated under reduced pressure to get the title compound as a pale yellow solid 6 (950 mg; crude), which was used in next step directly without further purification.

LCMS=[M+H]⁺: 484.31; Purity=70%.

### Synthesis of 1-(3-(5-((4-(4-morpholino-7H-pyrrolo[2,3-d]pyrimidin-6-yl)phenyl)amino)pyrimidin-2-yl)-3,8-diazabicyclo[3.2.1]octan-8-yl)prop-2-en-1-one (Compound 1083)

To an ice cold stirred solution of 2-(3,8-diazabicyclo[3.2.1]octan-3-yl)-N-(4-(4-morpholino-7H-pyrrolo[2,3-d]pyrimidin-6-yl)phenyl)pyrimidin-5-amine 6 (200 mg, 0.414 mmol) in THF (5 mL) was added K₃PO₄ (176 mg, 0.827 mmol) dissolved with water (2.5 mL) dropwise under N₂ atmosphere. Then, a solution of 3-chloropropanoyl chloride 7 (53 mg, 0.414 mmol) dissolved with THF (0.5 mL) was added dropwise. The resulting reaction mixture was stirred at RT for 1 h. After completion of starting material (acylation reaction, monitored by TLC), 2N aq. NaOH (2.5 mL, 4.96 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [2-98% 5 mM Ammonium bicarbonate in water/ACN; Column: X Bridge Sheild RP18 (19*250) 10μ] to get Compound 1083 (79 mg, 35%).

¹H NMR (400 MHz, DMSO-d₆): δ 12.03 (s, 1H), 8.31 (s, 2H), 8.14 (s, 1H), 7.93 (s, 1H), 7.70 (d, J=8.4 Hz, 2H), 6.92 (d, J=1.6 Hz, 1H), 6.83-6.75 (m, 3H), 6.20 (dd, J=16.4 Hz, 2.0 Hz, 1H), 5.72 (dd, J=10.4 Hz, 2.4 Hz, 1H), 4.69 (t, J=11.2 Hz, 2H), 4.35 (t, J=9.6 Hz, 2H), 3.86-3.84 (m, 4H), 3.75-3.72 (m, 4H), 3.02-2.94 (m, 2H), 1.94-1.91 (m, 1H), 1.82-1.65 (m, 3H).

LCMS=[M+H]⁺: 538.35; Purity=97.07%

### Example 146

### Compound 1084

### Preparation of 2-(azetidin-1-ylmethyl)-1-(7-(2,2,2-trifluoro-1-((4-(4-morpholino-7H-pyrrolo[2,3-d]pyrimidin-6-yl)phenyl)amino)ethyl)-2-azaspiro[3.5]nonan-2-yl)prop-2-en-1-one (Compound 1084)

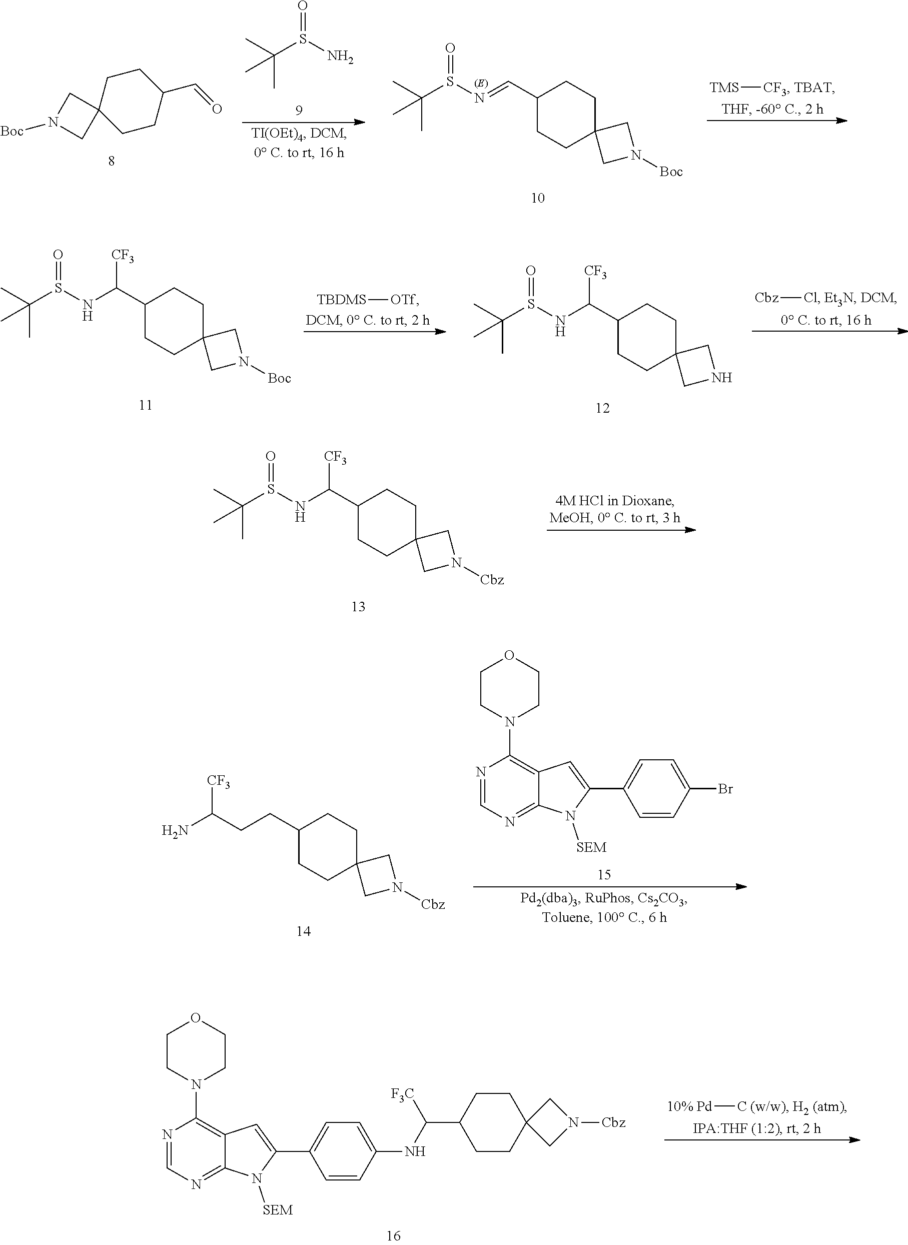

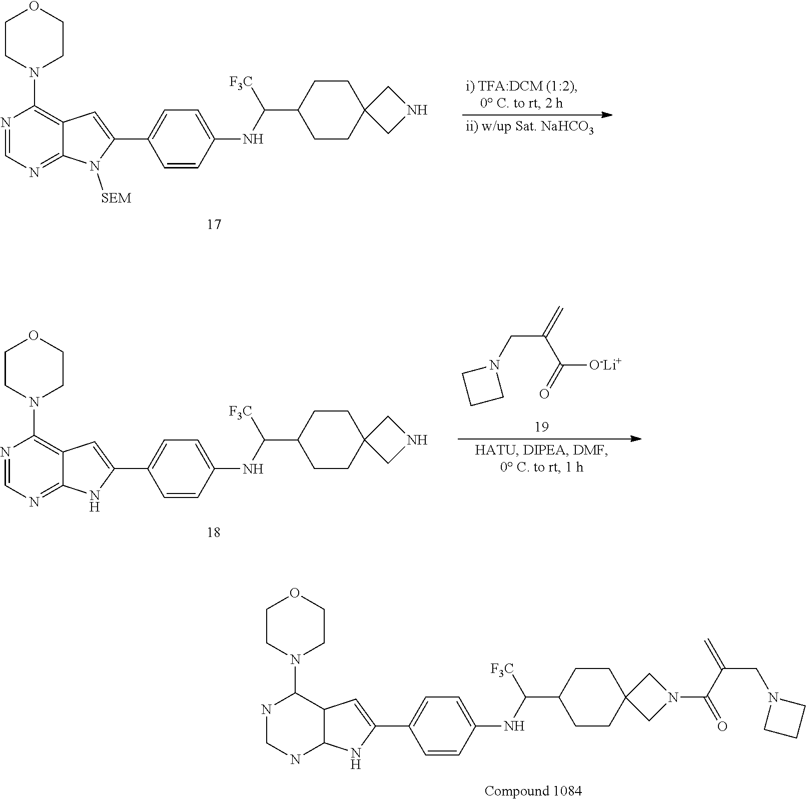

### Synthesis of tert-butyl (E)-7-(((tert-butylsulfinyl)imino)methyl)-2-azaspiro[3.5]nonane-2-carboxylate (10)

To a stirred solution of tert-butyl 7-formyl-2-azaspiro[3.5]nonane-2-carboxylate 8 (1 g, 3.95 mmol) in DCM (20 mL) were added 2-methylpropane-2-sulfinamide 9 (478 mg, 3.95 mmol) and Ti (OEt) 4 (1.68 mL, 7.89 mmol) sequentially at 0° C. under N₂ atmosphere. The reaction mixture was stirred at RT for 16 h. After completion of reaction (monitored by TLC), the reaction mixture was quenched with saturated brine solution (50 mL), filtered through celite pad and washed with DCM (100 mL). The filtrate was extracted with DCM (2×100 mL). The combined organic layer was dried over Na₂SO₄, filtered and evaporated under reduced pressure. The crude was purified by flash chromatography (silica gel 230-400 M, 24 g SNAP) using eluents 30-40% EtOAc in heptane to get the title compound as a white solid 10 (1.2 g, 85%).

LCMS=[M+H]⁺: 357.29; Purity=99%.

### Synthesis of tert-butyl 7-(1-((tert-butylsulfinyl)amino)-2,2,2-trifluoroethyl)-2-azaspiro[3.5]nonane-2-carboxylate (11)

To a stirred solution of tert-butyl (E)-7-(((tert-butylsulfinyl)imino)methyl)-2-azaspiro[3.5]nonane-2-carboxylate 10 (1.2 g, 3.37 mmol) in dry THF (30 mL) was added TBAT (2 g, 3.7 mmol) under N₂ atmosphere. The reaction mixture was cooled at −65° C., and a solution of TMS-CF₃ (0.76 mL, 5.05 mmol) dissolved in dry THF (2 mL) was added slowly and stirred for 1 h at same temperature. Then the reaction mixture was warmed to 0° C. and stirred for 2 h. After completion of reaction (monitored by TLC and LCMS), the reaction mixture was quenched with ice-cold water (50 mL) and extracted with EtOAc (3×70 mL). The combined organic layer was dried over Na₂SO₄, filtered and evaporated under reduced pressure. The crude was purified by flash chromatography (silica gel 230-400 M, 24 g SNAP) using eluents 25-30% EtOAc in heptane to get the title compound as a white solid 11 (900 mg, 56%).

LCMS=[M+H]⁺: 427.32; Purity=90%.

### Synthesis of 2-methyl-N-(2,2,2-trifluoro-1-(2-azaspiro[3.5]nonan-7-yl)ethyl) propane-2-sulfinamide (12)

To an ice cold stirred solution of tert-butyl 7-(1-((tert-butylsulfinyl)amino)-2,2,2-trifluoroethyl)-2-azaspiro[3.5]nonane-2-carboxylate 11 (900 mg, 2.11 mmol) in DCM (10 mL) was added TBDMS-OTf (0.55 mL, 2.53 mmol) at 0° C. under N₂ atmosphere. The reaction was stirred at RT for 2 h. After completion of reaction (monitored by TLC), the reaction mixture was quenched with sat. NaHCO₃ and extracted with 25% IPA in CHCl₃ (3×50 mL). The combined organic layer was dried over Na₂SO₄, filtered and concentrated under reduced pressure to get the title compound as a colorless sticky oil 12 (700 mg; crude), which was used in next step directly without further purification.

LCMS=[M+H]⁺: 327.19; Purity=80%.

### Synthesis of benzyl 7-(1-((tert-butylsulfinyl)amino)-2,2,2-trifluoroethyl)-2-azaspiro[3.5]nonane-2-carboxylate (13)

To a stirred solution of 2-methyl-N-(2,2,2-trifluoro-1-(2-azaspiro[3.5]nonan-7-yl)ethyl) propane-2-sulfinamide 12 (700 mg, 2.71 mmol) in DCM (10 mL) were added Et₃N (3.8 mL, 27.2 mmol) and Cbz-Cl (0.8 mL, 2.36 mmol) at 0° C. under N₂ atmosphere. The reaction was stirred at RT for 16 h. After completion of reaction (monitored by TLC), the reaction mixture was quenched with ice-cold water (50 mL) and extracted with DCM (3×50 mL). The combined organic layer was dried over Na₂SO₄, filtered and concentrated under reduced pressure. The crude was purified by flash chromatography (silica gel 230-400 M, 12 g SNAP) using eluents 25-30% EtOAc in heptane to get the title compound as light yellow sticky liquid 13 (700 mg, 72%).

LCMS=[M+H]⁺: 461.27; Purity=88%.

### Synthesis of benzyl 7-(1-amino-2,2,2-trifluoroethyl)-2-azaspiro[3.5]nonane-2-carboxylate (14)

To a stirred solution of benzyl 7-(1-((tert-butylsulfinyl)amino)-2,2,2-trifluoroethyl)-2-azaspiro[3.5]nonane-2-carboxylate 13 (700 mg, 1.52 mmol) in MeOH (10 mL) was added 4N HCl in 1,4-Dioxane (4 mL, 15.2 mmol) at 0° C. and stirred at RT for 3 h. After completion of reaction (monitored by TLC), the reaction mixture was concentrated under reduced pressure. The crude was basified with sat. NaHCO₃ to pH~8-9 and extracted with 25% IPA in CHCl₃ (3×60 mL). The combined organic layer was dried over Na₂SO₄, filtered and concentrated under reduced pressure to get the title compound as colorless liquid 14 (415 mg; crude), which was used in next step directly without further purification.

LCMS=[M+H]⁺: 357.18; Purity=85%.

### Synthesis of benzyl 7-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)-2-azaspiro[3.5]nonane-2-carboxylate (16)

To a stirred solution of 4-(6-(4-bromophenyl)-7-((2-(trimethylsilyl)ethoxy)methyl)-7H-pyrrolo[2,3-d]pyrimidin-4-yl)morpholine 15 (380 mg, 0.77 mmol) and benzyl 7-(1-amino-2,2,2-trifluoroethyl)-2-azaspiro[3.5]nonane-2-carboxylate 14 (415 mg, 1.53 mmol) in toluene (10 mL) was added Cs₂CO₃ (506 mg, 1.55 mmol) at RT. The reaction mixture was purged with N₂ gas for 10 min, followed by RuPhos (72.5 mg, 0.155 mmol) and Pd₂(dba)₃ (71 mg, 0.077 mmol) was added, and stirred at 100° C. for 6 h. After completion of reaction (monitored by TLC and LCMS), the reaction mixture was diluted with water (50 mL) and extracted with EtOAc (3×70 mL). The combined organic layer was dried over Na₂SO₄, filtered and evaporated under reduced pressure. The crude was purified by flash chromatography (silica gel 230-400 M, 12 g SNAP) using eluents 20-30% EtOAc in heptane to get the title compound as a brown solid 16 (450 mg, 76%).

LCMS=[M+H]⁺: 765.43; Purity=70%.

### Synthesis of 4-(4-morpholino-7-((2-(trimethylsilyl)ethoxy)methyl)-7H-pyrrolo[2,3-d]pyrimidin-6-yl)-N-(2,2,2-trifluoro-1-(2-azaspiro[3.5]nonan-7-yl)ethyl)aniline (17)

To a stirred solution of benzyl 7-(2,2,2-trifluoro-1-((4-(4-morpholino-7-((2-(trimethylsilyl)ethoxy)methyl)-7H-pyrrolo[2,3-d]pyrimidin-6-yl)phenyl)amino)ethyl)-2-azaspiro[3.5]nonane-2-carboxylate 16 (450 mg, 0.588 mmol) in a mixture of IPA (3 mL) and THF (6 mL) was added 10% Pd-C (450 mg, w/w) at RT under H₂ atmosphere. The reaction mixture was stirred at RT for 2 h. After completion of reaction (monitored by TLC and LCMS), the reaction mixture was filtered through celite pad and washed with MeOH (100 mL). The filtrate was concentrated under reduced pressure to get the title compound as colorless oil 17 (300 mg; crude), which was used in next step directly without further purification.

LCMS=[M+H]⁺: 631.44; Purity=75%.

### Synthesis of 4-(4-morpholino-7H-pyrrolo[2,3-d]pyrimidin-6-yl)-N-(2,2,2-trifluoro-1-(2-azaspiro[3.5]nonan-7-yl)ethyl)aniline (18)

To a stirred solution of 4-(4-morpholino-7-((2-(trimethylsilyl)ethoxy)methyl)-7H-pyrrolo[2,3-d]pyrimidin-6-yl)-N-(2,2,2-trifluoro-1-(2-azaspiro[3.5]nonan-7-yl)ethyl)aniline 17 (330 mg, 0.523 mmol) in DCM (5 mL) was added TFA (2.5 mL) at 0° C. under N₂ atmosphere. The reaction mixture was stirred at RT for 2 h. After completion of reaction (monitoring by TLC), the reaction mixture was concentrated under reduced pressure, triturated with Et₂O and dried under reduced pressure. The crude was reaction dissolved in 25% IPA in CHCl₃ (50 mL) and saturated NaHCO₃ (30 mL) was added and stirred for 30 min. After completion of reaction (monitored by LCMS), the reaction mixture was extracted with 25% IPA in CHCl₃ (2×50 mL) and washed with brine (2×30 mL). The organic layer was dried over Na₂SO₄, filtered and concentrated under reduced pressure to get the title compound as an off-white solid 18 (250 mg; crude), which was used in next step without further purification.

LCMS=[M+H]⁺: 501.26; Purity=90%.

### Synthesis of 2-(azetidin-1-ylmethyl)-1-(7-(2,2,2-trifluoro-1-((4-(4-morpholino-7H-pyrrolo[2,3-d]pyrimidin-6-yl)phenyl)amino)ethyl)-2-azaspiro[3.5]nonan-2-yl)prop-2-en-1-one (Compound 1084)

To an ice cold stirred solution of 4-(4-morpholino-7H-pyrrolo[2,3-d]pyrimidin-6-yl)-N-(2,2,2-trifluoro-1-(2-azaspiro[3.5]nonan-7-yl)ethyl)aniline 18 (225 mg, 0.449 mmol) and lithium 2-(azetidin-1-ylmethyl)acrylate 19 (198 mg, 1.35 mmol) in DMF (5 mL) were added DIPEA (0.3 mL, 1.80 mmol) and T3P (50% w/w)(0.85 mL, 1.35 mmol) under N₂ atmosphere. The resulting reaction mixture was stirred at RT for 1 h. After completion of reaction (monitored by TLC and LCMS), the reaction mixture was poured into ice-cold water (40 mL) and extracted with 25% IPA in CHCl₃ (3×60 mL). The combined organic layer was dried over Na2SO₄, filtered and evaporated under reduced pressure. The crude residue was purified by RP-HPLC [2-98% 0.1% TFA in Water/ACN; Column: X Select Fluoro Phenyl (19*250) 5μ] to get the desired Compound 1084 (30 mg, 10%).

¹H NMR (400 MHz, DMSO-d₆): δ 12.32 (brs, 1H), 9.65 (brs, 1H), 8.22 (s, 1H), 7.67-7.64 (m, 2H), 6.99-6.97 (m, 1H), 6.87-6.84 (m, 2H), 6.10-6.07 (m, 1H), 5.93 (d, J=6.8 Hz, 1H), 5.83 (d, J=9.6 Hz, 1H), 4.30-4.21 (m, 2H), 4.04-3.98 (m, 4H), 3.96-3.87 (m, 6H), 3.77-3.75 (m, 4H), 3.59 (d, J=18.8 Hz, 2H), 2.41-2.33 (m, 1H), 2.88-2.19 (m, 1H), 1.90-1.85 (m, 2H), 1.80-1.62 (m, 3H), 1.45 (q, J=12.8 Hz, 2H), 1.33-1.14 (m, 2H).

LCMS=[M+H]⁺: 624.46; Purity=97.17%.

### Example 138 and Example 147

### Compound 1072 and Compound 1085

### Preparation of (R)-1-(4-(5-(hydroxy (4-(4-morpholino-7H-pyrrolo[2,3-d]pyrimidin-6-yl)phenyl)methyl)pyrimidin-2-yl)piperazin-1-yl)prop-2-en-1-one (Compound 1072) and(S)-1-(4-(5-(hydroxy (4-(4-morpholino-7H-pyrrolo[2,3-d]pyrimidin-6-yl)phenyl)methyl)pyrimidin-2-yl)piperazin-1-yl)prop-2-en-1-one (Compound 1085)

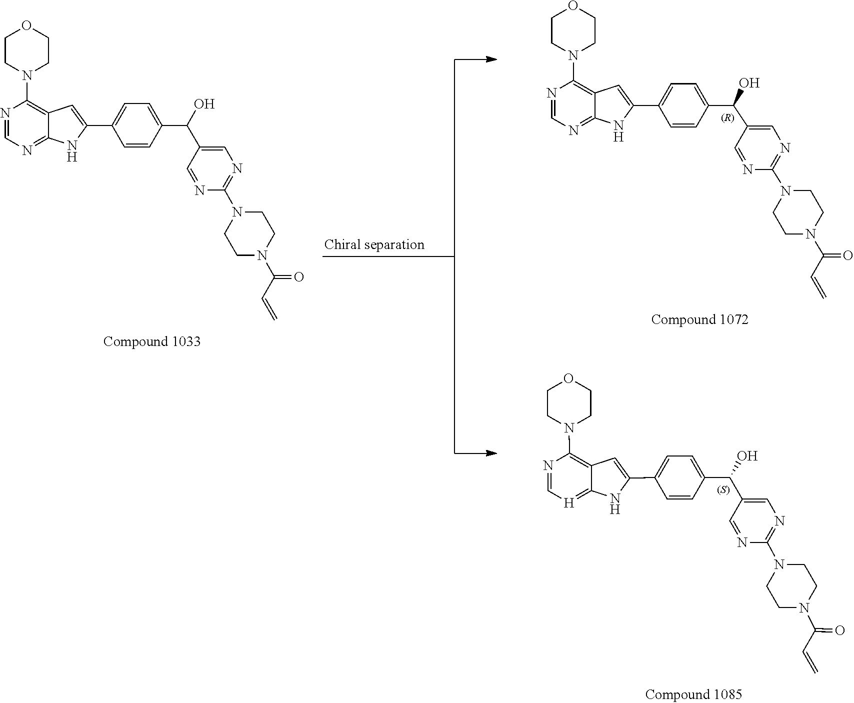

Chiral separation of Compound 1033 resulted in both enantiomers (R)-1-(4-(5-(hydroxy (4-(4-morpholino-7H-pyrrolo[2,3-d]pyrimidin-6-yl)phenyl)methyl)pyrimidin-2-yl)piperazin-1-yl)prop-2-en-1-one (Compound 1072) and(S)-1-(4-(5-(hydroxy (4-(4-morpholino-7H-pyrrolo[2,3-d]pyrimidin-6-yl)phenyl)methyl)pyrimidin-2-yl)piperazin-1-yl)prop-2-en-1-one (Compound 1085)

Note: Chirality arbitrarily assigned

Method for Chiral HPLC purification:

Column: CHIRALPAK-IE (21×250 mm; 5μ)

Mobile Phase: A) 0.1% TEA in McOH
B) DCM

Flow rate: Isocratic 24 mL/Min, wavelength: 250 nm & 330 nm

Analytical data of Compound 1072:

¹H NMR (400 MHz, DMSO-d₆): δ 12.33 (s, 1H), 8.35 (s, 2H), 8.20 (s, 1H), 7.87 (d, J=8.0 Hz, 2H), 7.44 (d, J=8.4 Hz, 2H), 7.19 (s, 1H), 6.82 (dd, J=16.8 Hz, 10.4 Hz, 1H), 6.13 (dd, J=16.8 Hz, 2.0 Hz, 1H), 5.97 (brs, 1H), 5.71-5.67 (m, 2H), 3.90-3.88 (m, 4H), 3.75-3.72 (m, 8H), 3.62-3.59 (m, 4H).

LCMS=[M+H]⁺: 527.33; Purity=96.11% and ee=99.8%.

Analytical data of Compound 1085:

¹H NMR (400 MHz, DMSO-d₆): δ 12.26 (s, 1H), 8.35 (s, 2H), 8.19 (s, 1H), 7.87 (d, J=8.4 Hz, 2H), 7.43 (d, J=8.4 Hz, 2H), 7.17 (s, 1H), 6.82 (dd, J=16.7 Hz, 10.4 Hz, 1H), 6.13 (dd, J=16.8 Hz, 2.2 Hz, 1H), 5.97 (brs, 1H), 5.71-5.67 (m, 2H), 3.89-3.87 (m, 4H), 3.76-3.73 (m, 8H), 3.62-3.59 (m, 4H).

LCMS=[M+H]⁺: 527.33; Purity=96.98% and ee=99%.

### Example 148

### Compound 1086

Preparation of N,N-diethyl-4-(4-(5-(hydroxy (4-(4-morpholino-7H-pyrrolo[2,3-d]pyrimidin-6-yl)phenyl)methyl)pyrimidin-2-yl)piperazin-1-yl)but-2-ynamide (Compound 1086)

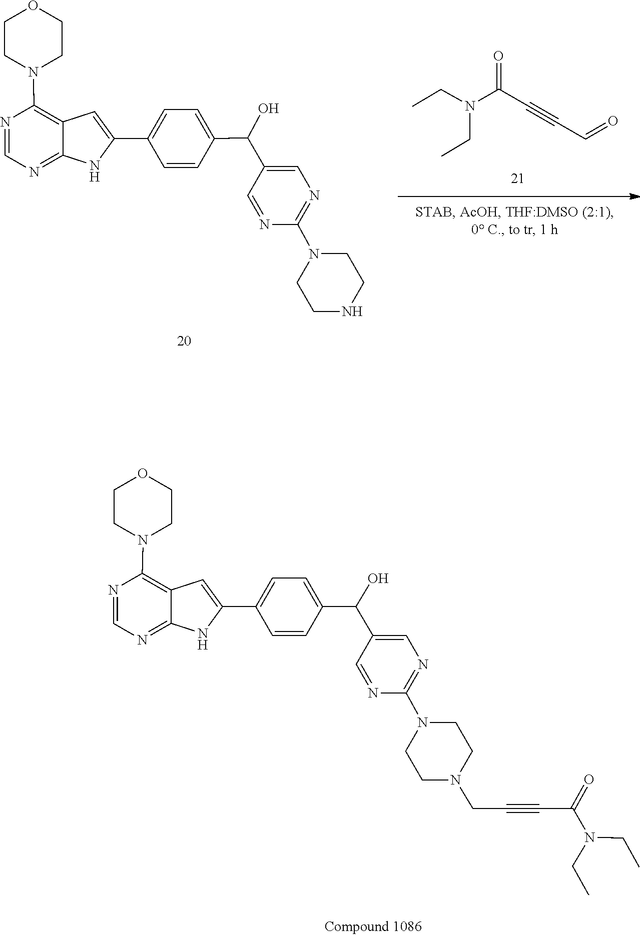 [SMILES: OC(c1ccc(-c2cc3c(N4CCOCC4)ncnc3[nH]2)cc1)c1cnc(N2CCNCC2)nc1 ; CCN(CC)C(=O)C#CC=O ; CCN(CC)C(=O)C#CCN1CCN(c2ncc(C(O)c3ccc(-c4cc5c(N6CCOCC6)ncnc5[nH]4)cc3)cn2)CC1]

To an ice cold stirred solution of (4-(4-morpholino-7H-pyrrolo[2,3-d]pyrimidin-6-yl)phenyl)(2-(piperazin-1-yl)pyrimidin-5-yl)methanol 20 (240 mg, 0.508 mmol) in a mixture of THF (8 mL) and DMSO (4 mL) were added N,N-diethyl-4-oxobut-2-ynamide 21 (120 mg, 0.762 mmol) and AcOH (60 μL, 1.02 mmol) under N₂ atmosphere stirred at RT for 30 min. Then, STAB (161 mg, 0.762 mmol) was added to the reaction mixture and stirred at RT for 1 h. After completion of reaction (LCMS monitoring), the reaction mixture was quenched with sat. NaHCO₃ (10 mL) and extracted with DCM (3×50 mL). The combined organic layer was dried over Na₂SO₄, filtered and concentrated under reduced pressure. The crude residue was purified by RP-HPLC [5-95% 0.1% TFA in Water/ACN; Column: Atlantis T3 (19*250) 10μ] to get Compound 1086 (15 mg, 5%).

¹H NMR (400 MHz, DMSO-d₆): δ 12.43 (s, 1H), 8.43 (s, 2H), 8.22 (s, 1H), 7.89 (d, J=8.4 Hz, 2H), 7.46 (d, J=8.4 Hz, 2H), 7.23 (s, 1H), 6.04 (brs, 1H), 5.70 (s, 1H), 4.10-4.02 (m, 2H), 3.91-3.88 (m, 6H), 3.80-3.76 (m, 8H), 3.43 (q, J=6.4 Hz, 2H), 3.25 (q, J=7.2 Hz, 2H), 3.16-2.96 (m, 2H), 1.03-0.95 (m, 6H).

LCMS=[M+H]⁺: 610.40; Purity=96.01%.

### Example 149

### Compound 1087

### Preparation of 1-(8-(5-((4-(4-morpholino-7H-pyrrolo[2,3-d]pyrimidin-6-yl)phenyl)amino)pyrimidin-2-yl)-3,8-diazabicyclo[3.2.1]octan-3-yl)prop-2-en-1-one (Compound 1087)

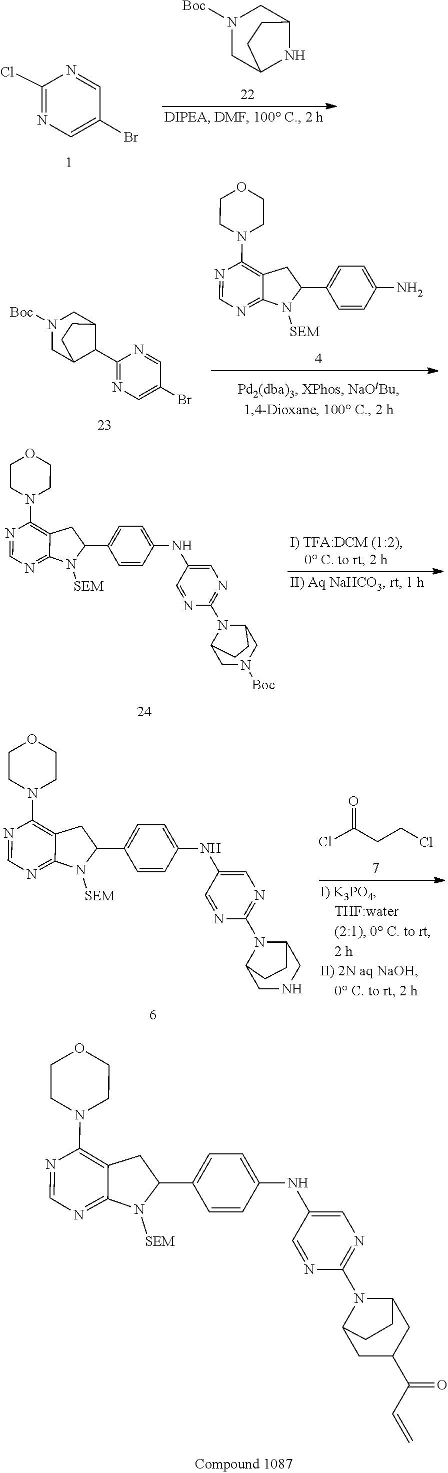

### Synthesis of tert-butyl 8-(5-bromopyrimidin-2-yl)-3,8-diazabicyclo[3.2.1]octane-3-carboxylate (23)

To a stirred solution of 5-bromo-2-chloropyrimidine 1 (2 g, 10.3 mmol) and tert-butyl 3,8-diazabicyclo[3.2.1]octane-3-carboxylate 22 (4.39 g, 20.7 mmol) in DMF (20 mL) was added DIPEA (5.4 mL, 31 mmol) at 0° C. The resulting reaction mixture was stirred at 100° C. for 2 h. After completion of reaction (monitored by TLC), the reaction mixture was quenched with ice cold-water (50 mL) and extracted with EtOAc (3×80 mL). The combined organic layer was dried over Na₂SO₄, filtered and evaporated under reduced pressure. The crude was purified by flash column chromatography (silica gel 230-400 M, 24 g SNAP) using eluents 10-12% EtOAc in heptane to get the title compound as white solid 23 (3.5 g, 91%).

LCMS=[M+H]⁺: 369.13; Purity=98.74%

### Synthesis of tert-butyl 8-(5-((4-(4-morpholino-7-((2-(trimethylsilyl)ethoxy)methyl)-7H-pyrrolo[2,3-d]pyrimidin-6-yl)phenyl)amino)pyrimidin-2-yl)-3,8-diazabicyclo[3.2.1]octane-3-carboxylate (24)

To a stirred solution of tert-butyl 8-(5-bromopyrimidin-2-yl)-3,8-diazabicyclo[3.2.1]octane-3-carboxylate 23 (1.5 g, 4.06 mmol) and 4-(4-morpholino-7-((2-(trimethylsilyl)ethoxy)methyl)-7H-pyrrolo[2,3-d]pyrimidin-6-yl)aniline 4 (2.07 g, 4.87 mmol) in 1,4-Dioxane (20 mL) was added NaO^{t}Bu (586 mg, 6.09 mmol) at RT. The resulting reaction mixture was degassed with N₂ gas for 10 min, followed by Pd₂(dba)₃ (372 mg, 0.406 mmol) and XPhos (194 mg, 0.406 mmol) were added. The resulting reaction mixture was stirred at 100° C. for 2 h. After completion of reaction (monitored by TLC and LCMS), the reaction mixture was diluted with water (50 mL) and extracted with EtOAc (3×80 mL). The combined organic layer was dried over Na₂SO₄, filtered and evaporated under reduced pressure. The crude was purified by combi-flash column chromatography (silica gel 230-400 M, 24 g SNAP) using eluents 15-20% EtOAc in heptane to get the title compound as a yellow solid 24 (2 g, 45%).

LCMS=[M−H]: 712.0; Purity=65%

### Example 150

### Compound 1092

### Preparation of 1-(8-(5-(hydroxy (4-(4-morpholino-7H-pyrrolo[2,3-d]pyrimidin-6-yl)phenyl)methyl)pyrimidin-2-yl)-3,8-diazabicyclo[3.2.1]octan-3-yl)prop-2-en-1-one (Compound 1092)

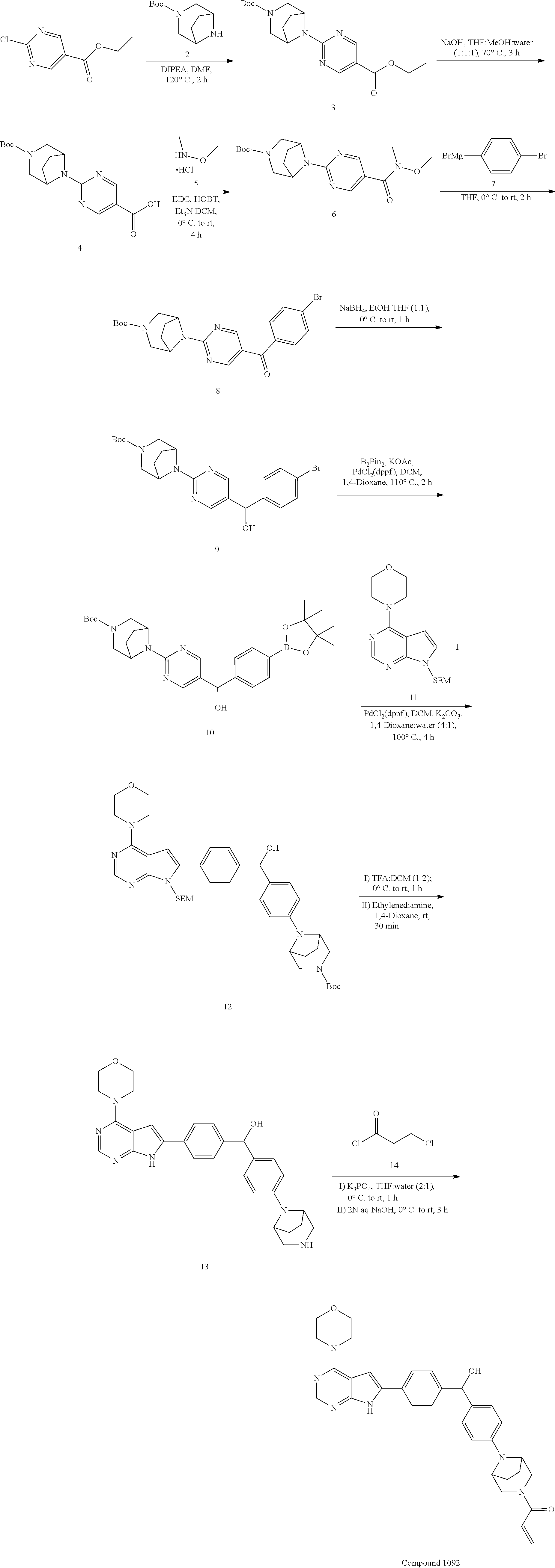

### Synthesis of tert-butyl 8-(5-(ethoxycarbonyl)pyrimidin-2-yl)-3,8-diazabicyclo[3.2.1]octane-3-carboxylate (3)

To a stirred solution of ethyl 2-chloropyrimidine-5-carboxylate 1 (1.5 g, 8.03 mmol) and tert-butyl 3,8-diazabicyclo[3.2.1]octane-3-carboxylate 2 (2.55 g, 12.05 mmol) in 1,4-Dioxane (20 mL) was added DIPEA (4.2 mL, 24.1 mmol) at RT under N₂ atmosphere. The reaction mixture was stirred at 120° C. for 2 h. After completion of reaction (monitored by TLC), the reaction mixture was concentrated under reduced pressure. The crude was purified by flash column chromatography (silica gel 230-400 M, 40 g SNAP) using eluents 25-30% EtOAc in heptane to get title compound as a white solid 3 (2.6 g, 90%).

LCMS=[M+H]⁺: 363.13; Purity=95%.

### Synthesis of 2-(3-(tert-butoxycarbonyl)-3,8-diazabicyclo[3.2.1]octan-8-yl)pyrimidine-5-carboxylic acid (4)

To an ice cold stirred solution of tert-butyl 8-(5-(ethoxycarbonyl)pyrimidin-2-yl)-3,8-diazabicyclo[3.2.1]octane-3-carboxylate 3 (2.6 g, 7.17 mmol) in THF (20 mL) and MeOH (20 mL) was added a aqueous solution of NaOH (574 mg, 7.17 mmol) dissolved in water (20 mL). The reaction mixture was stirred at 70° C. for 3 h. After completion of reaction (monitored by TLC), the reaction mixture was concentrated under reduced pressure. The crude was diluted with water (10 mL) and acidified with 1N HCl to pH~5. The obtained white solid precipitate was filtered, washed with ice cold water and dried under reduced pressure to get title compound as a white solid 4 (2.4 g; crude), which was used in next step directly without further purification.

LCMS=[M+H]⁺: 335.26; Purity=99%.

### Synthesis of tert-butyl 8-(5-(methoxy(methyl)carbamoyl)pyrimidin-2-yl)-3,8-diazabicyclo[3.2.1]octane-3-carboxylate (6)

To a stirred solution of 2-(3-(tert-butoxycarbonyl)-3,8-diazabicyclo[3.2.1]octan-8-yl)pyrimidine-5-carboxylic acid 4 (2.4 g, 7.18 mmol) in DCM (25 mL) were added Et₃N (3.91 mL, 28.7 mmol), HOBT (1.16 g, 8.61 mmol) and EDC·HCl (2.06 g, 10.8 mmol) sequentially to the reaction mixture at 0° C. under N₂ atmosphere. The resulting reaction mixture was stirred at RT for 1 h. Then, N,O-dimethylhydroxylamine hydrochloride 5 (840 mg, 8.61 mmol) was added to the reaction mixture and stirred at RT for 4 h. After completion of reaction (monitored by TLC and LCMS), the reaction mixture was poured into water (60 mL) and extracted with DCM (3×100 mL). The combined organic layer was washed with ice-cold brine solution (2×70 mL), dried over Na2SO₄, filtered and evaporated under reduced pressure. The crude was purified by the flash chromatography (silica gel 230-400 M, 40 g SNAP) using eluents 30-40% EtOAc in heptane to get the title compound as a white solid 6 (2.3 g, 85%).

LCMS=[M+H]⁺: 378.34; Purity=98%.

### Synthesis of tert-butyl 8-(5-(4-bromobenzoyl)pyrimidin-2-yl)-3,8-diazabicyclo[3.2.1]octane-3-carboxylate (8)

To an ice cold stirred solution of tert-butyl 8-(5-(methoxy(methyl)carbamoyl)pyrimidin-2-yl)-3,8-diazabicyclo[3.2.1]octane-3-carboxylate 6 (2.3 g, 6.09 mmol) in dry THF (30 mL) was added (4-bromophenyl) magnesium bromide 7 (49 mL, 24.7 mmol; 0.5 M in THF) dropwise to the reaction mixture under N2 atmosphere. The reaction mixture was stirred at RT for 2 h. After completion of reaction (monitored by TLC and LCMS), the reaction mixture was quenched with 1N HCl (60 mL) and extracted with EtOAc (3×100 mL). The combined organic layer was dried over Na₂SO₄, filtered and concentrated under reduced pressure. The crude was purified by flash column chromatography (silica gel 230-400 M, 24 g SNAP) using eluents 10-12% EtOAc in heptane to get the title compound as a white solid 8 (2 g, 70%).

LCMS=[M+H]⁺: 473.19; Purity=73%.

### Synthesis of tert-butyl 8-(5-((4-bromophenyl)(hydroxy)methyl)pyrimidin-2-yl)-3,8-diazabicyclo[3.2.1]octane-3-carboxylate (9)

To an ice cold stirred solution of tert-butyl 8-(5-(4-bromobenzoyl)pyrimidin-2-yl)-3,8-diazabicyclo[3.2.1]octane-3-carboxylate 8 (2 g, 4.23 mmol) in EtOH (15 mL) and THF (15 mL) was added NaBH₄ (320 mg, 8.45 mmol) at 0° C. under N₂ atmosphere. The reaction mixture was stirred at RT for 1 h. After completion of reaction (monitored by TLC), the reaction mixture was concentrated under reduced pressure. The crude was dissolved in water (60 mL) and extracted with DCM (3×80 mL). The combined organic layer was washed with brine solution (2×60 mL), dried over Na₂SO₄, filtered and evaporated under reduced pressure. The crude was purified by flash column chromatography (silica gel 230-400 M, 24 g SNAP) using eluents 25-30% EtOAc in heptane to get the title compound as a white solid 9 (1.9 g, 94%).

LCMS=[M+H]⁺: 475.11; Purity=77%.

### Synthesis of tert-butyl 8-(5-(hydroxy (4-(4,4,5,5-tetramethyl-1,3,2-dioxaborolan-2-yl)phenyl)methyl)pyrimidin-2-yl)-3,8-diazabicyclo[3.2.1]octane-3-carboxylate (10)

To a stirred solution of tert-butyl 8-(5-((4-bromophenyl)(hydroxy)methyl)pyrimidin-2-yl)-3,8-diazabicyclo[3.2.1]octane-3-carboxylate 9 (1.9 g, 4.01 mmol) and B₂Pin₂ (1.53 g, 6.01 mmol) in 1,4-Dioxane (20 mL) was added KOAc (1.2 g, 12 mmol). The resulting reaction mixture was degassed with N₂ gas for 10 min, followed by added PdCl₂(dppf)·DCM (330 mg, 0.401 mmol) and the reaction mixture was stirred at 110° C. for 2 h. After completion of reaction (monitored by TLC and LCMS), the reaction mixture was filtered through celite bed and washed with EtOAc (100 mL). The filtrate was evaporated under reduced pressure to get the title compound as brown sticky oil 10 (2 g; crude), which was used in next step directly without further purification.

LCMS=[M+H]⁺: 523.33; Purity=65%.

### Synthesis of tert-butyl 8-(5-(hydroxy (4-(4-morpholino-7-((2-(trimethylsilyl)ethoxy)methyl)-7H-pyrrolo[2,3-d]pyrimidin-6-yl)phenyl)methyl)pyrimidin-2-yl)-3,8-diazabicyclo[3.2.1]octane-3-carboxylate (12)

To a stirred solution of tert-butyl 8-(5-(hydroxy (4-(4,4,5,5-tetramethyl-1,3,2-dioxaborolan-2-yl)phenyl)methyl)pyrimidin-2-yl)-3,8-diazabicyclo[3.2.1]octane-3-carboxylate 10 (1.82 g, 3.47 mmol) and 4-(6-iodo-7-((2-(trimethylsilyl)ethoxy)methyl)-7H-pyrrolo[2,3-d]pyrimidin-4-yl)morpholine 11 (1 g, 2.17 mmol) in 1,4-Dioxane (24 mL) and water (6 mL) was added K₂CO₃ (900 mg, 6.51 mmol) at RT. The resulting reaction mixture was degassed with argon gas for 10 min, followed by added PdCl₂(dppf)·DCM (180 mg, 0.217 mmol) and the resulting reaction mixture was stirred at 100° C. for 4 h. After the completion of reaction (monitored by TLC), the reaction mixture was diluted with water (50 mL) and extracted with EtOAc (3×80 mL). The combined organic layer was washed with brine solution (2×80 mL), dried over Na₂SO₄, filtered and evaporated under reduced pressure. The crude was purified by flash column chromatography (silica gel 100- 200 mesh, 24 g SNAP) using eluents 30-40% EtOAc in heptane to get the desired compound as a brown solid 12 (1.1 g, 70%).

LCMS=[M+H]⁺: 729.43; Purity=83%.

### Synthesis of (2-(3,8-diazabicyclo[3.2.1]octan-8-yl)pyrimidin-5-yl)(4-(4-morpholino-7H-pyrrolo[2,3-d]pyrimidin-6-yl)phenyl)methanol (13)

To an ice cold stirred solution of tert-butyl 8-(5-(hydroxy (4-(4-morpholino-7-((2-(trimethylsilyl)ethoxy)methyl)-7H-pyrrolo[2,3-d]pyrimidin-6-yl)phenyl)methyl)pyrimidin-2-yl)-3,8-diazabicyclo[3.2.1]octane-3-carboxylate 12 (1.1 g, 1.51 mmol) in DCM (20 mL) was added TFA (10 mL) dropwise under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 1,4-Dioxane (10 mL) and ethylenediamine (151 μL, 2.26 mmol) was added and stirred at RT for 30 min. After completion of reaction (monitored by LCMS), the reaction was poured into ice-cold water (50 mL) and extracted with 25% IPA in CHCl₃ (3×60 mL). The combined organic layer was dried over Na₂SO₄, filtered and concentrated under reduced pressure to get the title compound as a pale yellow solid 13 (800 mg; crude), which was used in next step directly without further purification.

LCMS=[M+H]⁺: 499.20; Purity=64%.

### Synthesis of 1-(8-(5-(hydroxy (4-(4-morpholino-7H-pyrrolo[2,3-d]pyrimidin-6-yl)phenyl)methyl)pyrimidin-2-yl)-3,8-diazabicyclo[3.2.1]octan-3-yl)prop-2-en-1-one (Compound 1092)

To an ice cold stirred solution of (2-(3,8-diazabicyclo[3.2.1]octan-8-yl)pyrimidin-5-yl)(4-(4-morpholino-7H-pyrrolo[2,3-d]pyrimidin-6-yl)phenyl)methanol 13 (300 mg, 0.602 mmol) in THF (8 mL) was added K₃PO₄ (255 mg, 1.2 mmol) dissolved in water (4 mL) under N₂ atmosphere. Then a solution of 3-chloropropanoyl chloride 14 (70 μL, 0.722 mmol) in THF (1 mL) was added dropwise to the reaction mixture and stirred at RT for 1 h. After completion of starting material (acylation reaction, monitored by TLC), 2N aq. NaOH (3.6 mL, 7.22 mmol) was added to the reaction mixture and stirred at RT for 3 h. After completion of reaction (complete elimination, monitored by TLC and LCMS), the reaction mixture was diluted with water (50 mL) and extracted with 2-Me THF (3×60 mL). The combined organic layer was dried over Na₂SO₄, filtered and concentrated under reduced pressure. The crude was purified by RP-HPLC [2-98% 5 mM Ammonium bicarbonate in water/ACN; Column: X Select CSH C18 (19*250) 10μ] to get Compound 1092 (59 mg, 18%).

¹H NMR (400 MHz, DMSO-d₆): δ 12.22 (s, 1H), 8.37 (s, 2H), 8.17 (s, 1H), 7.87 (d, J=8.0 Hz, 2H), 7.44 (d, J=8.4 Hz, 2H), 7.15 (d, J=1.6 Hz, 1H), 6.73 (dd, J=16.8 Hz, 10.4 Hz, 1H), 6.09 (dd, J=16.8 Hz, 2.0 Hz, 1H), 5.99 (d, J=4.0 Hz, 1H), 5.68-5.65 (m, 2H), 4.70 (brs, 2H), 4.23 (d, J=12.8 Hz, 1H), 3.88-3.84 (m, 5H), 3.75-3.73 (m, 4H), 3.29-3.26 (m, 1H), 2.82 (d, J=13.2 Hz, 1H), 1.89-1.78 (m, 2H), 1.66-1.56 (m, 2H).

LCMS=[M+H]⁺: 553.43; Purity=94.08%

### Example 151

### Compound 1093

### Preparation of 1-(3-(5-(hydroxy (4-(4-morpholino-7H-pyrrolo[2,3-d]pyrimidin-6-yl)phenyl)methyl)pyrimidin-2-yl)-3,8-diazabicyclo[3.2.1]octan-8-yl)prop-2-en-1-one (Compound 1093)

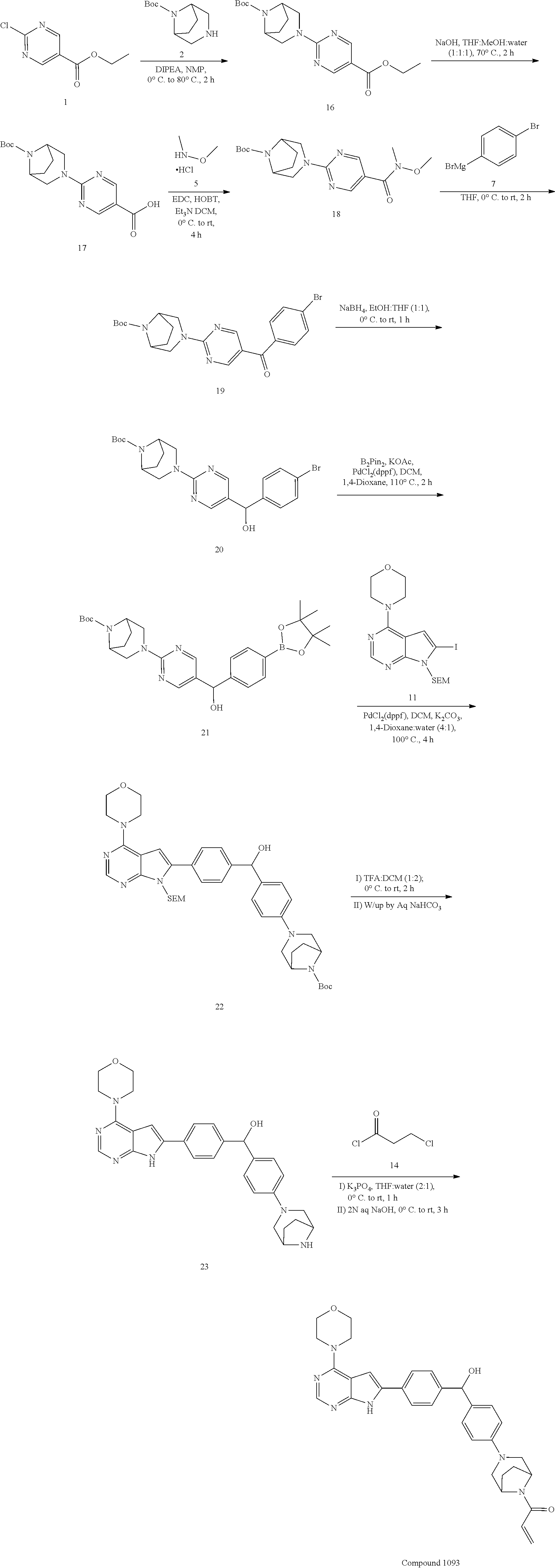

### Synthesis of tert-butyl 3-(5-(ethoxycarbonyl)pyrimidin-2-yl)-3,8-diazabicyclo[3.2.1]octane-8-carboxylate (16)

To a stirred solution of ethyl 2-chloropyrimidine-5-carboxylate 1 (5 g, 26.8 mmol) and tert-butyl 3,8-diazabicyclo[3.2.1]octane-8-carboxylate 15 (6.83 g, 32.2 mmol) in NMP (100 mL) was DIPEA (9.5 mL, 53.6 mmol) at 0° C. under N₂ atmosphere. The reaction mixture was stirred at 80° C. for 2 h. After completion of reaction (monitored by TLC), the reaction mixture was poured into ice-cold water (200 mL) and extracted with EtOAc (3×100 mL). The combined organic layer washed ice-cold brine (2×100 mL), dried over Na₂SO₄, filtered and concentrated under reduced pressure. The crude was purified by flash column chromatography (silica gel 230-400 M, 40 g SNAP) using eluents 25-30% EtOAc in heptane to get title compound as a white solid 16 (9 g, 92%).

LCMS=[M+H]⁺: 363.30; Purity=85%.

### Synthesis of 2-(8-(tert-butoxycarbonyl)-3,8-diazabicyclo[3.2.1]octan-3-yl)pyrimidine-5-carboxylic acid (17)

To an ice cold stirred solution of tert-butyl 3-(5-(ethoxycarbonyl)pyrimidin-2-yl)-3,8-diazabicyclo[3.2.1]octane-8-carboxylate 16 (5 g, 13.8 mmol) in THF (30 mL) and MeOH (30 mL) was added aqueous solution of NaOH (1.1 g, 27.6 mmol) dissolved in H₂O (30 mL). The reaction mixture was stirred at 70° C. for 2 h. After completion of reaction (monitored by TLC), the reaction mixture was concentrated under reduced pressure. The crude was diluted with water (20 mL) and acidified with 1N HCl to pH~5. The obtained white solid precipitate was filtered, washed with ice cold water and dried under reduced pressure to get title compound as a white solid 17 (4.6 g; crude), which was used in next step directly without further purification.

LCMS=[M+H]⁺: 335.12; Purity=98%.

### Synthesis of tert-butyl 3-(5-(methoxy(methyl)carbamoyl)pyrimidin-2-yl)-3,8-diazabicyclo[3.2.1]octane-8-carboxylate (18)

To an ice cold stirred solution of 2-(8-(tert-butoxycarbonyl)-3,8-diazabicyclo[3.2.1]octan-3-yl)pyrimidine-5-carboxylic acid 17 (4.6 g, 13.8 mmol) in DCM (50 mL) were added Et₃N (7.67 mL, 55 mmol), HOBt (2.23 g, 16.5 mmol) and EDC·HCl (3.96 g, 20.6 mmol) sequentially to the reaction mixture under N₂ atmosphere. The reaction mixture was stirred at RT for 4 h. Then, N,O-dimethylhydroxylamine hydrochloride 5 (1.61 g, 16.5 mmol) was added to the reaction mixture and stirred at RT for 4 h. After completion of reaction (monitored by TLC and LCMS), the reaction mixture was poured into water (100 mL) and extracted with DCM (3×100 mL). The combined organic layer was washed with brine solution (2×100 mL), dried over Na₂SO₄, filtered and evaporated under reduced pressure. The crude was purified over the flash chromatography (silica gel 230-400 M, 80 g SNAP) using eluents 30-40% EtOAc in heptane to get the title compound as a white solid 18 (2.7 g, 52%).

LCMS=[M+H]⁺: 378.10; Purity=98%.

### Synthesis of tert-butyl 3-(5-(4-bromobenzoyl)pyrimidin-2-yl)-3,8-diazabicyclo[3.2.1]octane-8-carboxylate (19)

To an ice cold stirred solution of tert-butyl 3-(5-(methoxy(methyl)carbamoyl)pyrimidin-2-yl)-3,8-diazabicyclo[3.2.1]octane-8-carboxylate 18 (2.7 g, 7.15 mmol) and dry THF (20 mL) was added (4-bromophenyl) magnesium bromide 7 (57.2 mL, 28.6 mmol; 0.5 M in THF) dropwise to the reaction mixture under N₂ atmosphere. The reaction mixture was stirred at RT for 2 h. After completion of reaction (monitored by TLC and LCMS), the reaction mixture was quenched with 1N HCl (60 mL) and extracted with EtOAc (3×100 mL). The combined organic layer was dried over Na₂SO₄, filtered and concentrated under reduced pressure. The crude was purified by flash column chromatography (silica gel 230-400 M, 24 g SNAP) using eluents 10-12% EtOAc in heptane to get the title compound as a white solid 19 (1.9 g, 56%).

LCMS=[M+H]⁺: 473.09; Purity=82%.

### Synthesis of tert-butyl 3-(5-((4-bromophenyl)(hydroxy)methyl)pyrimidin-2-yl)-3,8-diazabicyclo[3.2.1]octane-8-carboxylate (20)

To an ice cold stirred solution of tert-butyl 3-(5-(4-bromobenzoyl)pyrimidin-2-yl)-3,8-diazabicyclo[3.2.1]octane-8-carboxylate 19 (1.9 g, 4.02 mmol) in EtOH (20 mL) and THF (20 mL) was added NaBH₄ (304 mg, 8.03 mmol) at same temperature under N₂ atmosphere. The reaction mixture was stirred at RT for 1 h. After completion of reaction (monitored by TLC), the reaction mixture was concentrated under reduced pressure. The crude residue was diluted with water (50 mL) and extracted with DCM (3×100 mL). The combined organic layer was washed with brine solution (2×100 mL), dried over Na₂SO₄, filtered and evaporated under reduced pressure. The crude was purified by flash column chromatography (silica gel 230-400 M, 24 g SNAP) using eluents 25-30% EtOAc in heptane to get the title compound as a white solid 20 (1.9 g, 98%).

LCMS=[M+H]⁺: 475.31; Purity=82%.

### Synthesis of tert-butyl 3-(5-(hydroxy (4-(4,4,5,5-tetramethyl-1,3,2-dioxaborolan-2-yl)phenyl)methyl)pyrimidin-2-yl)-3,8-diazabicyclo[3.2.1]octane-8-carboxylate (21)

To a stirred solution of tert-butyl 3-(5-((4-bromophenyl)(hydroxy)methyl)pyrimidin-2-yl)-3,8-diazabicyclo[3.2.1]octane-8-carboxylate 20 (1.9 g, 4.01 mmol) and B₂Pin₂ (1.53 g, 6.01 mmol) in 1,4-Dioxane (20 mL) was added KOAc (1.8 g, 12 mmol). The resulting reaction mixture was degassed with N₂ gas for 10 min, followed by added PdCl₂(dppf)·DCM (327 mg, 0.401 mmol) and the reaction mixture was stirred at 110° C. for 2 h. After completion of reaction (monitored by TLC and LCMS), the reaction mixture was filtered through celite bed and washed with EtOAc (100 mL). The filtrate was evaporated under reduced pressure to get the title compound as brown sticky oil 21 (2.2 g; crude), which was used in next step directly without further purification.

LCMS=[M+H]⁺: 523.33; Purity=65%.

### Synthesis of tert-butyl 3-(5-(hydroxy (4-(4-morpholino-7-((2-(trimethylsilyl)ethoxy)methyl)-7H-pyrrolo[2,3-d]pyrimidin-6-yl)phenyl)methyl)pyrimidin-2-yl)-3,8-diazabicyclo[3.2.1]octane-8-carboxylate (22)

To a stirred solution of tert-butyl ((3R)-1-(5-(hydroxy (4-(4,4,5,5-tetramethyl-1,3,2-dioxaborolan-2-yl)phenyl)methyl)pyrimidin-2-yl)piperidin-3-yl)carbamate 21 (2.2 g, 4.23 mmol) and 4-(6-iodo-7-((2-(trimethylsilyl)ethoxy)methyl)-7H-pyrrolo[2,3-d]pyrimidin-4-yl)morpholine 11 (1.3 g, 2.82 mmol) in 1,4-dioxane (40 mL) and water (10 mL) was added K₂CO₃ (1.16 g, 8.46 mmol)) at RT. The resulting reaction mixture was degassed with argon gas for 15 min, followed by was added PdCl₂(dppf)·DCM (230 mg, 0.282 mmol) and the resulting reaction mixture was stirred at 100° C. for 4 h. After the completion of reaction (monitored by TLC), the reaction mixture was diluted with water (100 mL) and extracted with EtOAc (3×100 mL). The combined organic layer was washed with brine solution (2×100 mL), dried over Na₂SO₄, filtered and evaporated under reduced pressure. The crude was purified by flash column chromatography (silica gel 230-400 M, 40 g SNAP) using eluents 50-60% EtOAc in heptane to get the title compound as a brown solid 22 (900 mg, 43%).

LCMS=[M+H]⁺: 729.54; Purity=85%.

### Synthesis of (2-(3,8-diazabicyclo[3.2.1]octan-3-yl)pyrimidin-5-yl)(4-(4-morpholino-7H-pyrrolo[2,3-d]pyrimidin-6-yl)phenyl)methanol (23)

To an ice cold stirred solution of tert-butyl 3-(5-hydroxy(4-(4-morpholine-7-((2-(trimethylsilyl)ethoxy)methyl)-7H-pyrrolo[2,3-d]pyrimidin-6-yl)phenyl)methyl)pyrimidin-2-yl)-3,8-diazabicyclo; 22 (600 mg, 0.837 mmol) in DCM (6 mL) was added TFA (3 mL) dropwise under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The obtained crude was dissolved in 25% IPA in CHCl₃ (10 mL) and saturated NaHCO₃ (10 mL) was added and stirred at RT for 1 h. Then, the reaction mixture was extracted with 25% IPA in CHCl₃ (3×50 mL), dried over Na₂SO₄, filtered and concentrated under reduced pressure to get the title compound as a brownish semi solid 23 (350 mg; crude), which was used in next step directly without further purification.

LCMS=[M+H]⁺: 499.36; Purity=80%.

### Synthesis of 1-(3-(5-(hydroxy (4-(4-morpholino-7H-pyrrolo[2,3-d]pyrimidin-6-yl)phenyl)methyl)pyrimidin-2-yl)-3,8-diazabicyclo[3.2.1]octan-8-yl)prop-2-en-1-one (Compound 1093)

To an ice cold stirred solution of {2-(3,8-diazabicyclo[3.2.1]oct-3-yl)-5-pyrimidinyl}[p-(4-morpholino-1H-1,5,7-triazainden-2-yl)phenyl]methanol 23 (150 mg, 0.301 mmol) in THF (4 mL) was added K₃PO₄ (128 mg, 0.602 mmol) dissolved in water (2 mL) under N₂ atmosphere. Then a solution of 3-chloropropanoyl chloride 14 (35 μL, 0.361 mmol) in THF (0.5 mL) was added dropwise to the reaction mixture and stirred at RT for 1 h. After completion of starting material (acylation reaction, monitored by TLC), 2N aq. NaOH (1.8 mL, 3.61 mmol) was added to the reaction mixture and stirred at RT for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [2-98% 5 mM Ammonium bicarbonate in water/ACN; Column: X Select CSH C18 (19*250) 10μ] to get Compound 1093 (32 mg, 20%).

¹H NMR (400 MHz, DMSO-d₆): δ 12.21 (s, 1H), 8.32 (s, 2H), 8.17 (s, 1H), 7.87 (d, J=8.4 Hz, 2H), 7.43 (d, J=8.4 Hz, 2H), 7.15 (d. J=2.0 Hz, 1H), 6.76 (dd, J=16.8 Hz, 10.4 Hz, 1H), 6.18 (dd, J=16.8 Hz, 2.0 Hz, 1H), 5.96 (d, J=4.0 Hz, 1H), 5.71 (dd, J=10.4 Hz, 2.0 Hz, 1H), 5.66 (d, J=4.0 Hz, 1H), 4.65 (t, J=8.8 Hz, 2H), 4.40-4.36 (m, 2H), 3.88-3.86 (m, 4H), 3.75-3.73 (m, 4H), 2.94 (q, J=12.4 Hz, 2H), 1.90-1.82 (m, 1H), 1.77-1.72 (m, 1H), 1.66-1.53 (m, 2H).

LCMS=[M+H]⁺: 553.47; Purity=99.56%.

### Synthesis of 2-(3,8-diazabicyclo[3.2.1]octan-8-yl)-N-(4-(4-morpholino-7H-pyrrolo[2,3-d]pyrimidin-6-yl)phenyl)pyrimidin-5-amine (25)

To an ice cold stirred solution of tert-butyl 8-(5-((4-(4-morpholino-7-((2-(trimethylsilyl)ethoxy)methyl)-7H-pyrrolo[2,3-d]pyrimidin-6-yl)phenyl)amino)pyrimidin-2-yl)-3,8-diazabicyclo[3.2.1]octane-3-carboxylate 24 (1.9 g, 2.66 mmol) in DCM (20 mL) was added TFA (10 mL) dropwise under N₂ atmosphere. The reaction mixture was stirred at RT for 2 h. After completion of reaction (TLC and LCMS monitoring), the reaction mixture was concentrated under reduced pressure triturated with Et₂O and dried under reduced pressure. The crude was reaction dissolved in 25% IPA in CHCl₃ (50 mL) and saturated NaHCO₃ (30 mL) was added and stirred for 30 min. After completion of reaction (monitored by LCMS), the reaction mixture was extracted with 25% IPA in CHCl₃ (2×50 mL) and washed with brine (2×30 mL). The organic layer was dried over Na₂SO₄, filtered and concentrate under reduced pressure to get the title compound as yellow solid 25 (1.4 g, crude), which was directly used in the next step without any purification.

LCMS=[M+H]⁺: 484.28; Purity=75%.

### Synthesis of 1-(8-(5-((4-(4-morpholino-7H-pyrrolo[2,3-d]pyrimidin-6-yl)phenyl)amino)pyrimidin-2-yl)-3,8-diazabicyclo[3.2.1]octan-3-yl)prop-2-en-1-one (Compound 1087)

To an ice cold stirred solution of 2-(3,8-diazabicyclo[3.2.1]octan-8-yl)-N-(4-(4-morpholino-7H-pyrrolo[2,3-d]pyrimidin-6-yl)phenyl)pyrimidin-5-amine 25 (300 mg, 0.620 mmol) in THF (4 mL) was added K₃PO₄ (263 mg, 1.24 mmol) dissolved with water (2 mL) dropwise under N₂ atmosphere. Then, a solution of 3-chloropropanoyl chloride 7 (94.5 mg, 0.744 mmol) dissolved with THF (0.5 mL) was added dropwise. The resulting reaction mixture was stirred at RT for 1 h. After completion of starting material (acylation reaction, monitored by TLC), 2N aq. NaOH (3.8 mL, 7.44 mmol) was added to the reaction mixture at 0° C. and stirred at RT for 2 h. After completion of reaction (complete elimination, monitored by TLC and LCMS), the reaction mixture was diluted with water (50 mL) and extracted with 2-MeTHF (3×60 mL). The combined organic layer was dried over Na₂SO₄, filtered and concentrated under reduced pressure. The crude was purified by RP-HPLC [5-95% 5 mM Ammonium bicarbonate in water/ACN; Column: X Select CSH C18 (19*250) 10μ] to get Compound 1087 (82 mg, 24%).

¹H NMR (400 MHz, DMSO-d₆): δ 12.69 (s, 1H), 8.36 (s, 2H), 8.29 (s, 1H), 8.07 (brs, 1H), 7.74 (d, J=8.8 Hz, 2H), 7.15 (s, 1H), 6.88 (d, J=8.4 Hz, 2H), 6.77 (dd, J=16.4 Hz, 10.4 Hz, 1H), 6.12 (dd, J=16.4 Hz, 2.4 Hz, 1H), 5.69 (dd, J=10.4 Hz, 2.4 Hz, 1H), 4.70 (brs, 2H), 4.25 (d, J=13.6 Hz, 1H), 3.93-3.87 (m, 5H), 3.80-3.78 (m, 4H), 3.36 (d, J=12.4 Hz, 1H), 2.91 (d, J=12.8 Hz, 1H), 1.94-1.82 (m, 2H), 1.70-1.61 (m, 2H).

LCMS=[M+H]⁺: 538.35; Purity=98.57%.

### Example 152

### Compound 1094

### 4-(((R)-3-(2-(((2R,6S)-2,6-dimethylpiperidin-1-yl)methyl)acrylamido)piperidin-1-yl)methyl)-N-(4-(4-morpholino-7H-pyrrolo[2,3-d]pyrimidin-6-yl)phenyl)picolinamide

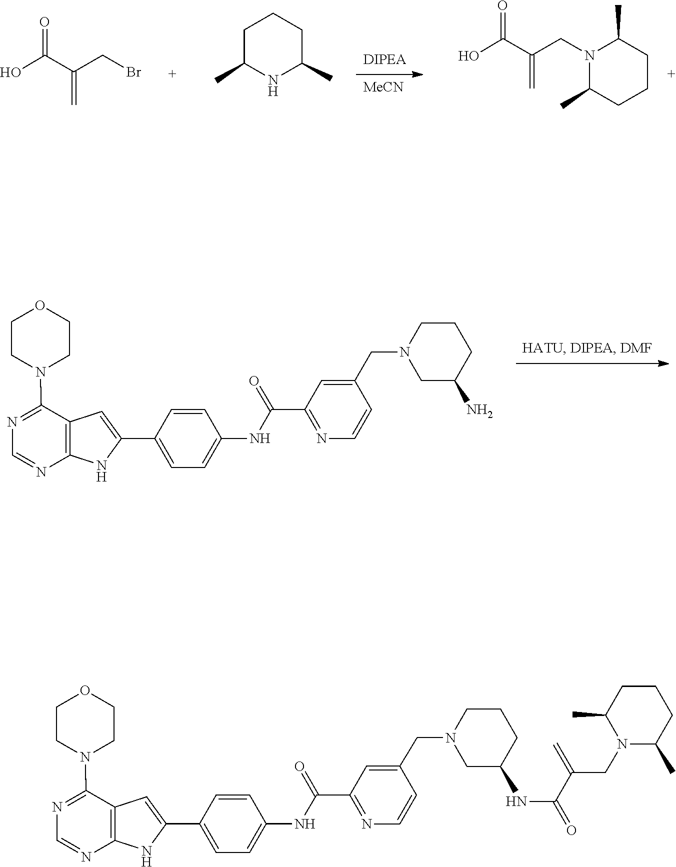

Step 1:2-(((2R,6S)-2,6-dimethylpiperidin-1-yl)methyl)acrylic acid

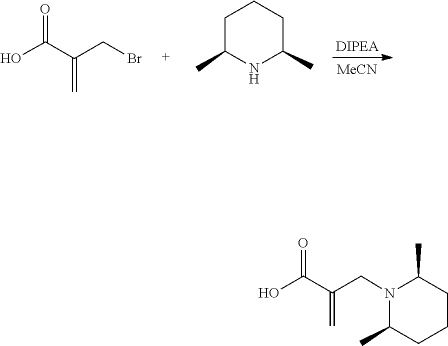

A solution of 2-(bromomethyl)prop-2-enoic acid (100 mg, 0.606 mmol), (2R,6S)-2,6-dimethylpiperidine (89.1 mg, 0.787 mmol) and N,N-diisopropylethylamine (0.32 mL, 1.82 mmol) in acetonitrile (2 mL) was stirred at 35° C. for 18 hours, providing a solution of 2-(((2R,6S)-2,6-dimethylpiperidin-1-yl)methyl)acrylic acid (119 mg, 0.603 mmol, 99%) in acetonitrile. The solution (0.26 M) was used in the next step without workup. LCMS=[M−H]−: 196.00.

Step 2:4-(((R)-3-(2-(((2R,6S)-2,6-dimethylpiperidin-1-yl)methyl)acrylamido)piperidin-1-yl)methyl)-N-(4-(4-morpholino-7H-pyrrolo[2,3-d]pyrimidin-6-yl)phenyl)picolinamide

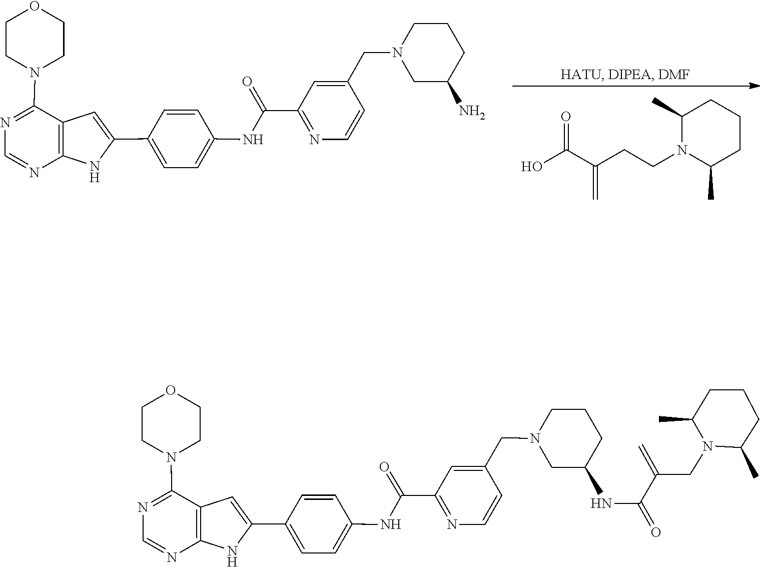 [SMILES: N[C@@H]1CCCN(Cc2ccnc(C(=O)Nc3ccc(-c4cc5c(N6CCOCC6)ncnc5[nH]4)cc3)c2)C1 ; C=C(CCN1[C@H](C)CCC[C@@H]1C)C(=O)O ; C=C(CN1[C@H](C)CCC[C@@H]1C)C(=O)N[C@@H]1CCCN(Cc2ccnc(C(=O)Nc3ccc(-c4cc5c(N6CCOCC6)ncnc5[nH]4)cc3)c2)C1]

A solution of 2-(((2R,6S)-2,6-dimethylpiperidin-1-yl)methyl)acrylic acid (0.18 mL, 0.0482 mmol) (0.26 M, reaction mixture without workup) was added to a solution of O-(7-azabenzotriazol-1-yl)-N,N,N′,N′-tetramethyluronium hexafluorophosphate (19.8 mg, 0.0523 mmol) and N,N-diisopropylethylamine (0.42 mL, 0.242 mmol) in N,N-dimethylformamide (2 mL). After 5 minutes (R)-4-((3-aminopiperidin-1-yl)methyl)-N-(4-(4-morpholino-7H-pyrrolo[2,3-d]pyrimidin-6-yl)phenyl)picolinamide trihydrochloride (25 mg, 0.0402 mmol) was added. After 45 minutes the mixture was diluted with ethyl acetate (30 mL) and washed with 5% lithium chloride (2×10 mL), saturated sodium bicarbonate (2×10 mL) and saturated ammonium chloride (10 mL). The organic phase was dried over sodium sulfate and the solvent was removed under reduced pressure. The residue was subjected to preparative HPLC(5-85% acetonitrile/water, 0.1% TFA). The fractions containing product were combined and subjected to lyophilization, providing 4-(((R)-3-(2-(((2R,6S)-2,6-dimethylpiperidin-1-yl)methyl)acrylamido)piperidin-1-yl)methyl)-N-(4-(4-morpholino-7H-pyrrolo[2,3-d]pyrimidin-6-yl)phenyl)picolinamide trifluoroacetate (11.6 mg, 0.0137 mmol, 34%). LCMS=[M+H]+: 692.40; Purity=95%, ¹H NMR (400 MHz, MeOD) δ 8.74 (dd, J=4.9, 0.8 Hz, 1H), 8.29 (s, 1H), 8.20 (s, 1H), 7.90-7.82 (m, 2H), 7.81-7.73 (m, 2H), 7.68 (dd, J=5.0, 1.8 Hz, 1H), 7.16 (s, 1H), 6.11-6.04 (m, 1H), 5.95 (s, 1H), 4.46-4.36 (m, 2H), 4.21-4.05 (m, 2H), 3.99 (t, J=4.9 Hz, 4H), 3.92 (s, 1H), 3.82 (dd, J=5.7, 4.1 Hz, 4H), 3.50-3.38 (m, 2H), 3.38-3.27 (m, 1H), 3.14-2.97 (m, 2H), 2.97-2.78 (m, 1H), 2.06-1.91 (m, 2H), 1.91-1.79 (m, 2H), 1.79-1.70 (m, 1H), 1.70-1.55 (m, 4H), 1.55-1.38 (m, 2H), 1.38-1.28 (m, 3H), 1.28-1.14 (m, 4H).

### Example 153

### Compound 1095

### 4-(((3R)-3-(2-((8-azabicyclo[3.2.1]octan-8-yl)methyl)acrylamido)piperidin-1-yl)methyl)-N-(4-(4-morpholino-7H-pyrrolo[2,3-d]pyrimidin-6-yl)phenyl)picolinamide

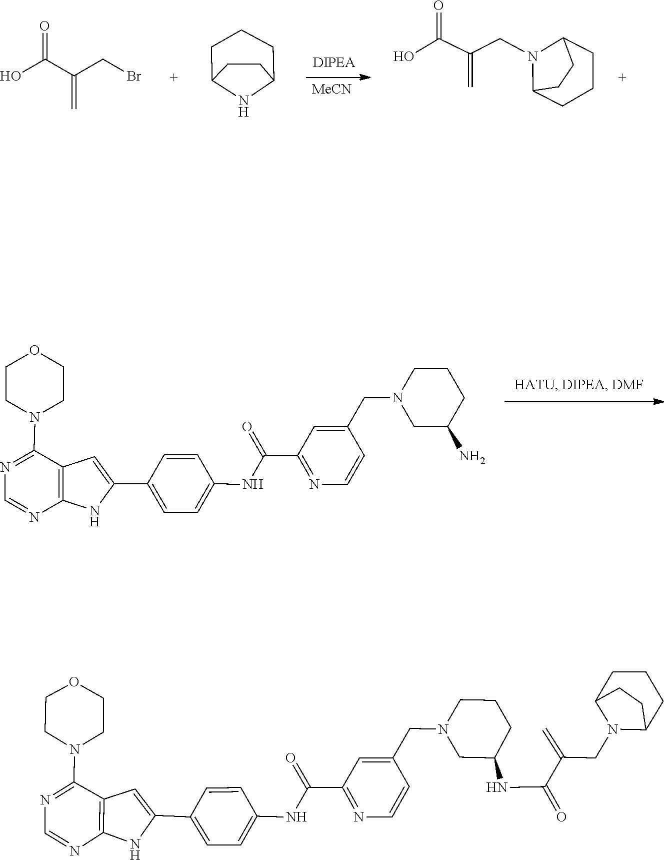

Step 1:2-((8-azabicyclo[3.2.1]octan-8-yl)methyl)acrylic acid

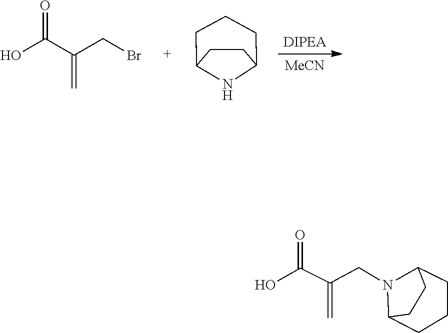

A solution of 2-(bromomethyl)prop-2-enoic acid (100 mg, 0.606 mmol), 8-azabicyclo[3.2.1]octane hydrochloride (116.3 mg, 0.787 mmol) and N,N-diisopropylethylamine (0.32 mL, 1.82 mmol) in acetonitrile (2 mL) was stirred at 35° C. for 18 hours, providing a solution of 2-((8-azabicyclo[3.2.1]octan-8-yl)methyl)acrylic acid (118 mg, 0.604 mmol, 99%) in acetonitrile. The solution (0.26 M) was used in the next step without workup. LCMS=[M−H]−: 195.80

Step 2:4-(((3R)-3-(2-((8-azabicyclo[3.2.1]octan-8-yl)methyl)acrylamido)piperidin-1-yl)methyl)-N-(4-(4-morpholino-7H-pyrrolo[2,3-d]pyrimidin-6-yl)phenyl)picolinamide

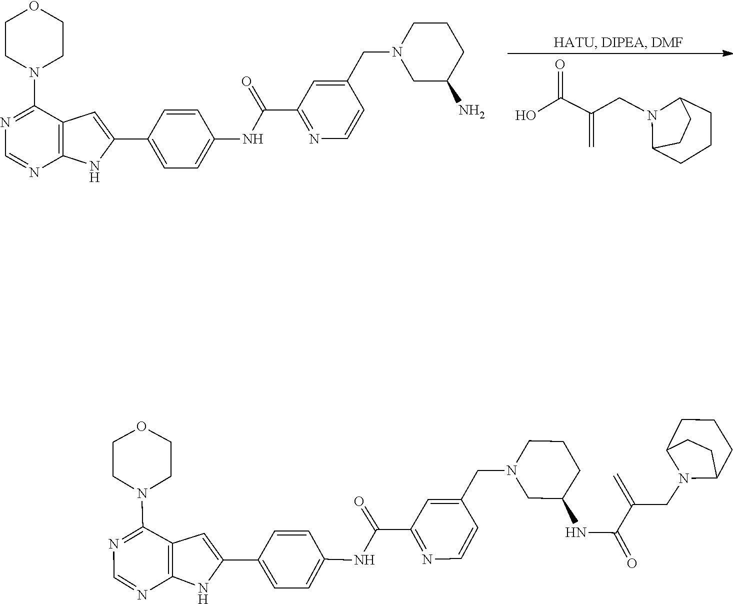 [SMILES: N[C@@H]1CCCN(Cc2ccnc(C(=O)Nc3ccc(-c4cc5c(N6CCOCC6)ncnc5[nH]4)cc3)c2)C1 ; C=C(CN1C2CCCC1CC2)C(=O)O ; C=C(CN1C2CCCC1CC2)C(=O)N[C@@H]1CCCN(Cc2ccnc(C(=O)Nc3ccc(-c4cc5c(N6CCOCC6)ncnc5[nH]4)cc3)c2)C1]

A solution of 2-((8-azabicyclo[3.2.1]octan-8-yl)methyl)acrylic acid (0.18 mL, 0.0482 mmol)(0.26 M, reaction mixture without workup) was added to a solution of O-(7-azabenzotriazol-1-yl)-N,N,N′,N′-tetramethyluronium hexafluorophosphate (19.8 mg, 0.0523 mmol) and N,N-diisopropylethylamine (0.42 mL, 0.242 mmol) in N,N-dimethylformamide (2 mL). After 5 minutes (R)-4-((3-aminopiperidin-1-yl)methyl)-N-(4-(4-morpholino-7H-pyrrolo[2,3-d]pyrimidin-6-yl)phenyl)picolinamide trihydrochloride (25 mg, 0.0402 mmol) was added. After 45 minutes the mixture was diluted with ethyl acetate (30 mL) and washed with 5% lithium chloride (2×10 mL), saturated sodium bicarbonate (2×10 mL) and saturated ammonium chloride (10 mL). The organic phase was dried over sodium sulfate and the solvent was removed under reduced pressure. The residue was subjected to preparative HPLC(5-85% acetonitrile/water, 0.1% TFA). The fractions containing product were combined and submitted to lyophilization, providing 4-(((3R)-3-(2-((8-azabicyclo[3.2.1]octan-8-yl)methyl)acrylamido)piperidin-1-yl)methyl)-N-(4-(4-morpholino-7H-pyrrolo[2,3-d]pyrimidin-6-yl)phenyl)picolinamide trifluoroacetate (9.3 mg, 0.0110 mmol, 27%). LCMS=[M+H]⁺: 690.40; Purity=95%, ¹H NMR (400 MHz, MeOD) δ 8.74 (d, 1H), 8.29 (s, 1H), 8.20 (s, 1H), 7.91-7.83 (m, 2H), 7.82-7.74 (m, 2H), 7.67 (dd, J=5.0, 1.7 Hz, 1H), 7.17 (s, 1H), 6.19 (s, 1H), 5.97 (s, 1H), 4.45-4.37 (m, 2H), 4.21-4.08 (m, 1H), 3.99 (dd, J=5.9, 3.9 Hz, 4H), 3.86-3.75 (m, 6H), 3.73 (s, 2H), 3.49-3.37 (m, 1H), 3.37-3.27 (m, 1H), 3.12-2.97 (m, 1H), 2.97-2.79 (m, 1H), 2.23-2.06 (m, 2H), 2.05-1.95 (m, 2H), 1.95-1.90 (m, 1H), 1.90-1.87 (m, 1H), 1.87-1.74 (m, 3H), 1.74-1.59 (m, 4H), 1.59-1.46 (m, 1H).

### Example 154

### Compound 1096

### 4-(((R)-3-((R)-3-amino-2-methylenebutanamido)piperidin-1-yl)methyl)-N-(4-(4-morpholino-7H-pyrrolo[2,3-d]pyrimidin-6-yl)phenyl)picolinamide

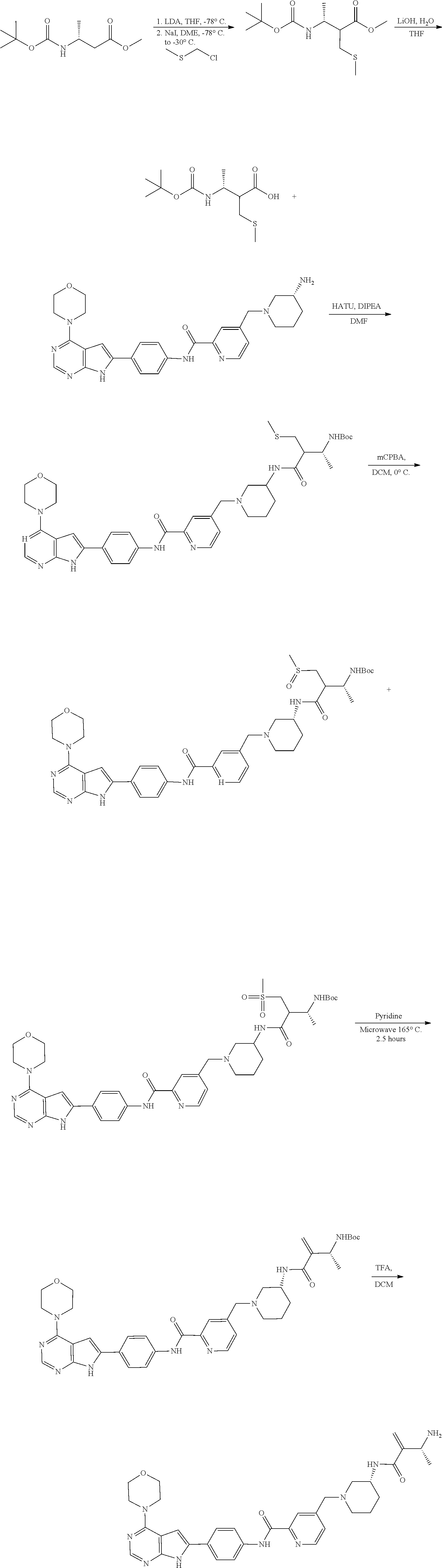

Step 1: Methyl (3R)-3-((tert-butoxycarbonyl)amino)-2-((methylthio)methyl) butanoate

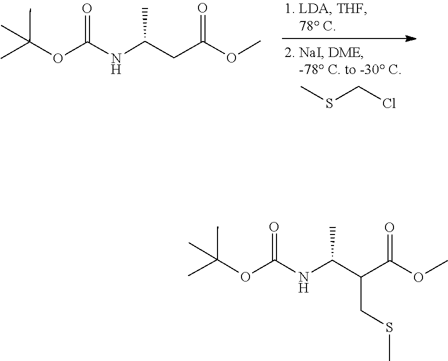

A solution of methyl (3R)-3-(tert-butoxycarbonylamino)butanoate (500 mg, 2.30 mmol) in tetrahydrofuran (5 mL) was added dropwise to lithium diisopropylamide (1.0 M, 5.5 mL, 5.52 mmol) at −78° C. and stirred for 1 hour. In a separate flask chloromethyl methyl sulfide (0.46 mL, 5.52 mmol) was added to a solution of sodium iodide (862 mg, 5.75 mmol) in dimethoxyethane (50 mL) and stirred at 21° C. for 1 hour.

This mixture was added to the LDA solution at −78° C. over a period of 10 minutes. The temperature was not allowed to go above −60° C. The mixture was stirred for 2 hours at −30° C. The reaction was quenched with saturated ammonium chloride (20 mL) and extracted with dichloromethane (3×50 mL). The combined organic phases were washed with saturated ammonium chloride (2×50 mL) and dried over sodium sulfate. The solvent was removed under reduced pressure and the residue was subjected to flash chromatography (0-40% ethyl acetate/hexanes). The fractions containing product were combined and the solvent was removed under reduced pressure, providing methyl (3R)-3-(tert-butoxycarbonylamino)-2-(methylsulfanylmethyl) butanoate (149 mg, 0.537 mmol, 23%). LCMS=[M+Na]+: 300.10;

Step 2: (3R)-3-((tert-butoxycarbonyl)amino)-2-((methylthio)methyl) butanoic acid

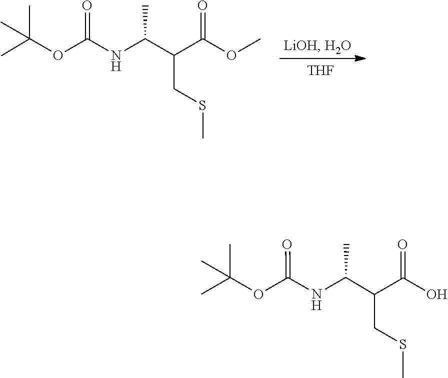

A solution of methyl (3R)-3-(tert-butoxycarbonylamino)-2-(methylsulfanylmethyl) butanoate (149 mg, 0.537 mmol) and lithium hydroxide monohydrate (43 mg, 1.02 mmol) in tetrahydrofuran (3 mL), methanol (0.5 mL) and water (0.5 mL) was stirred at 21° C. for 22 hours. The reaction was neutralized with hydrochloric acid (1.0 M, 1.9 mL, 1.88 mmol) and diluted with brine (10 ml). The mixture was extracted with ethyl acetate (5×10 mL). The combined organic phases were dried over sodium sulfate and the solvent was removed under reduced pressure, providing (3R)-3-(tert-butoxycarbonylamino)-2-(methylsulfanylmethyl) butanoic acid (135 mg, 0.512 mmol, 95%). LCMS=[M+Na]+: 286.10.

Step 3: tert-butyl ((2R)-3-((methylthio)methyl)-4-(((R)-1-((2-((4-(4-morpholino-7H-pyrrolo[2,3-d]pyrimidin-6-yl)phenyl)carbamoyl)pyridin-4-yl)methyl)piperidin-3-yl)amino)-4-oxobutan-2-yl)carbamate

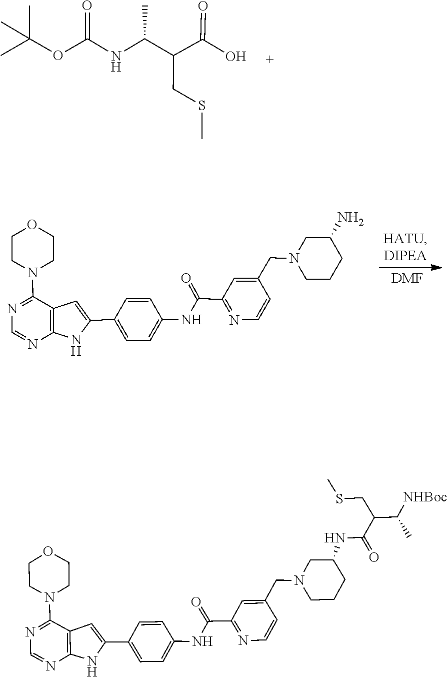

### N,N-Diisopropylethylamine (0.096 mL, 0.550 mmol) was added to a solution of

(3R)-3-(tert-butoxycarbonylamino)-2-(methylsulfanylmethyl) butanoic acid (28 mg, 0.105 mmol), O-(7-azabenzotriazol-1-yl)-N,N,N′.N′-tetramethyluronium hexafluorophosphate (45 mg, 0.119 mmol) in N,N-dimethylformamide (2 mL). After 5 minutes (R)-4-((3-aminopiperidin-1-yl)methyl)-N-(4-(4-morpholino-7H-pyrrolo[2,3-d]pyrimidin-6-yl)phenyl)picolinamide trihydrochloride (57 mg, 0.0916 mmol) was added. After 30 minutes the reaction was diluted with ethyl acetate (30 mL) and washed with 5% lithium chloride (2×10 mL), saturated sodium bicarbonate (2×10 mL) and saturated ammonium chloride (10 mL). The organic phase was dried over sodium sulfate and the solvent was removed under reduced pressure, providing tert-butyl ((2R)-3-((methylthio)methyl)-4-(((R)-1-((2-((4-(4-morpholino-7H-pyrrolo[2,3-d]pyrimidin-6-yl)phenyl)carbamoyl)pyridin-4-yl)methyl)piperidin-3-yl)amino)-4-oxobutan-2-yl)carbamate (67 mg, 0.0877 mmol, 95% yield). LCMS=[M+H]⁺: 758.40.

Step 4: tert-butyl ((2R)-3-((methylsulfinyl)methyl)-4-(((R)-1-((2-((4-(4-morpholino-7H-pyrrolo[2,3-d]pyrimidin-6-yl)phenyl)carbamoyl)pyridin-4-yl)methyl)piperidin-3-yl)amino)-4-oxobutan-2-yl)carbamate and tert-butyl ((2R)-3-((methylsulfonyl)methyl)-4-(((R)-1-((2-((4-(4-morpholino-7H-pyrrolo[2,3-d]pyrimidin-6-yl)phenyl)carbamoyl)pyridin-4-yl)methyl)piperidin-3-yl)amino)-4-oxobutan-2-yl)carbamate

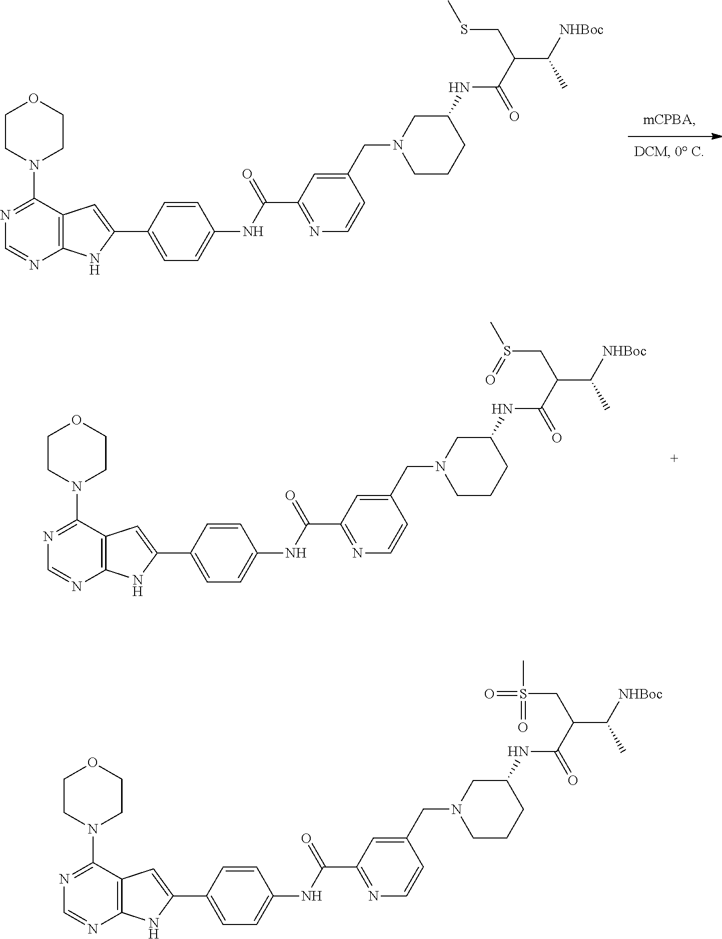 [SMILES: CSCC(C(=O)N[C@@H]1CCCN(Cc2ccnc(C(=O)Nc3ccc(-c4cc5c(N6CCOCC6)ncnc5[nH]4)cc3)c2)C1)[C@@H](C)NC(=O)OC(C)(C)C ; C[C@@H](NC(=O)OC(C)(C)C)C(CS(C)=O)C(=O)N[C@@H]1CCCN(Cc2ccnc(C(=O)Nc3ccc(-c4cc5c(N6CCOCC6)ncnc5[nH]4)cc3)c2)C1 ; C[C@@H](NC(=O)OC(C)(C)C)C(CS(C)(=O)=O)C(=O)N[C@@H]1CCCN(Cc2ccnc(C(=O)Nc3ccc(-c4cc5c(N6CCOCC6)ncnc5[nH]4)cc3)c2)C1]

3-Chloroperoxybenzoic acid (20 mg, 0.0877 mmol) was added to a solution of tert-butyl ((2R)-3-((methylthio)methyl)-4-(((R)-1-((2-((4-(4-morpholino-7H-pyrrolo[2,3-d]pyrimidin-6-yl)phenyl)carbamoyl)pyridin-4-yl)methyl)piperidin-3-yl)amino)-4-oxobutan-2-yl)carbamate (67 mg, 0.0877 mmol) in dichloromethane (5 mL) at 0° C. After 30 minutes the mixture was diluted with dichloromethane (20 mL) and quenched with saturates bicarbonate (10 mL). The mixture was extracted with dichloromethane (3×50 ml) and ethyl acetate (30 ml). The combined organic phases were dried over sodium sulfate and the solvent was removed under reduced pressure, providing a mixture of tert-butyl ((2R)-3-((methylsulfinyl)methyl)-4-(((R)-1-((2-((4-(4-morpholino-7H-pyrrolo[2,3-d]pyrimidin-6-yl)phenyl)carbamoyl)pyridin-4-yl)methyl)piperidin-3-yl)amino)-4-oxobutan-2-yl)carbamate and tert-butyl ((2R)-3-((methylsulfonyl)methyl)-4-(((R)-1-((2-((4-(4-morpholino-7H-pyrrolo[2,3-d]pyrimidin-6-yl)phenyl)carbamoyl)pyridin-4-yl)methyl)piperidin-3-yl)amino)-4-oxobutan-2-yl)carbamate (59 mg, 0.0741 mmol, 84%). LCMS=[M+H]+: 774.40, 790.40.

Step 5: tert-butyl ((R)-3-(((R)-1-((2-((4-(4-morpholino-7H-pyrrolo[2,3-d]pyrimidin-6-yl)phenyl)carbamoyl)pyridin-4-yl)methyl)piperidin-3-yl)carbamoyl)but-3-en-2-yl)carbamate

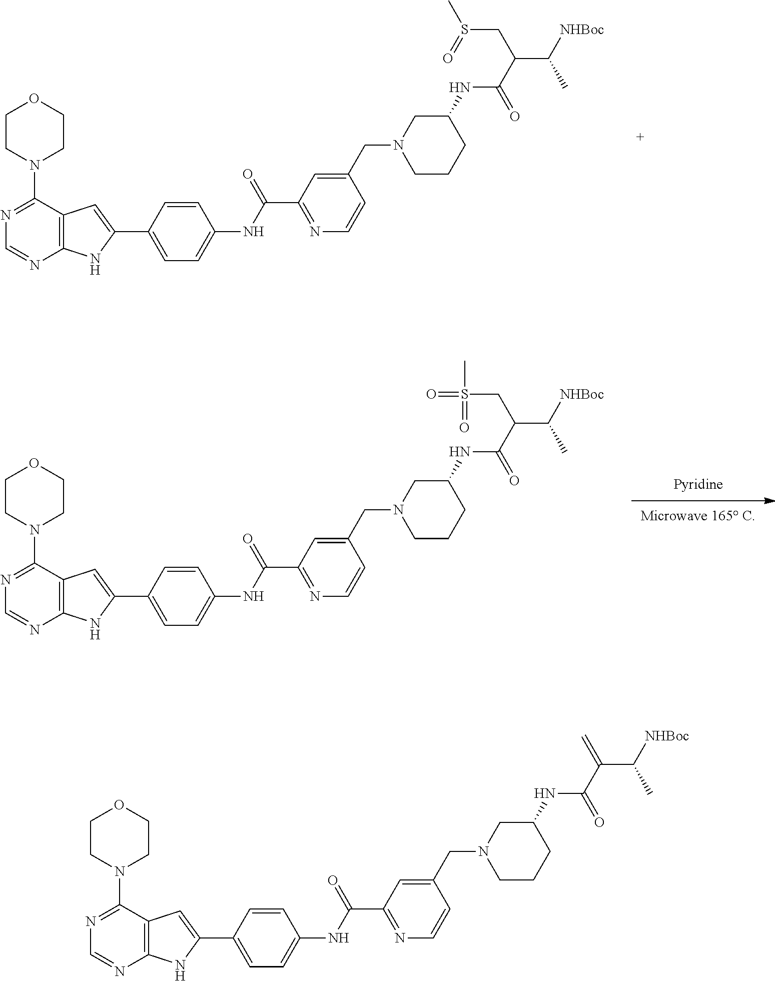 [SMILES: C[C@@H](NC(=O)OC(C)(C)C)C(CS(C)=O)C(=O)N[C@@H]1CCCN(Cc2ccnc(C(=O)Nc3ccc(-c4cc5c(N6CCOCC6)ncnc5[nH]4)cc3)c2)C1 ; C[C@@H](NC(=O)OC(C)(C)C)C(CS(C)(=O)=O)C(=O)N[C@@H]1CCCN(Cc2ccnc(C(=O)Nc3ccc(-c4cc5c(N6CCOCC6)ncnc5[nH]4)cc3)c2)C1 ; C=C(C(=O)N[C@@H]1CCCN(Cc2ccnc(C(=O)Nc3ccc(-c4cc5c(N6CCOCC6)ncnc5[nH]4)cc3)c2)C1)[C@@H](C)NC(=O)OC(C)(C)C]

A solution of tert-butyl ((2R)-3-((methylsulfinyl)methyl)-4-(((R)-1-((2-((4-(4-morpholino-7H-pyrrolo[2,3-d]pyrimidin-6-yl)phenyl)carbamoyl)pyridin-4-yl)methyl)piperidin-3-yl)amino)-4-oxobutan-2-yl)carbamate and tert-butyl ((2R)-3-((methylsulfonyl)methyl)-4-(((R)-1-((2-((4-(4-morpholino-7H-pyrrolo[2,3-d]pyrimidin-6-yl)phenyl)carbamoyl)pyridin-4-yl)methyl)piperidin-3-yl)amino)-4-oxobutan-2-yl)carbamate (36 mg, 0.0461 mmol) in pyridine (3 mL) was heated in a microwave reactor at 165° C. for 2.5 hours. The volatiles were removed under reduced pressure, providing tert-butyl ((R)-3-(((R)-1-((2-((4-(4-morpholino-7H-pyrrolo[2,3-d]pyrimidin-6-yl)phenyl)carbamoyl)pyridin-4-yl)methyl)piperidin-3-yl)carbamoyl)but-3-en-2-yl)carbamate (24 mg, 0.0333 mmol, 74% yield). LCMS=[M+H]⁺: 710.50.

Step 6:4-(((R)-3-((R)-3-amino-2-methylenebutanamido)piperidin-1-yl)methyl)-N-(4-(4-morpholino-7H-pyrrolo[2,3-d]pyrimidin-6-yl)phenyl)picolinamide

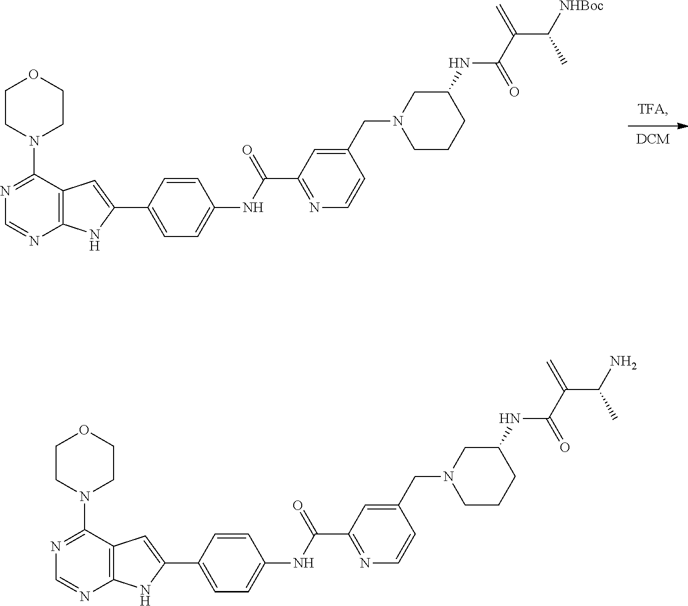

A solution of tert-butyl ((R)-3-(((R)-1-((2-((4-(4-morpholino-7H-pyrrolo[2,3-d]pyrimidin-6-yl)phenyl)carbamoyl)pyridin-4-yl)methyl)piperidin-3-yl)carbamoyl)but-3-en-2-yl)carbamate (32 mg, 0.0451 mmol) and trifluoroacetic acid (0.69 mL, 9.02 mmol) in dichloromethane (3 mL) was stirred at 21° C. for 15 minutes. The volatiles were removed under reduced pressure. The residue was subjected to preparative HPLC (5-85% acetonitrile/water, 0.1% trifluoroacetic acid). The fractions containing product were combined and subjected to lyophilization, providing 4-(((R)-3-((R)-3-amino-2-methylenebutanamido)piperidin-1-yl)methyl)-N-( 4-(4-morpholino-7H-pyrrolo[2,3-d]pyrimidin-6-yl)phenyl)picolinamide trifluoroacetate (26 mg, 0.0352 mmol, 78%). LCMS=[M+H]⁺: 610.40, Purity 95%, ¹H NMR (400 MHz, MeOD) δ 8.74 (d, J=4.9 Hz, 1H), 8.29 (d, J=1.7 Hz, 1H), 8.20 (s, 1H), 7.89-7.81 (m, 2H), 7.79-7.71 (m, 2H), 7.68 (dd, J=5.0, 1.8 Hz, 1H), 7.15 (s, 1H), 6.00-5.96 (m, 1H), 5.73 (t, J=1.1 Hz, 1H), 4.49-4.37 (m, 2H), 4.15 (m, 1H), 4.08 (q, J=6.9 Hz, 1H), 3.99 (m, 1H), 3.82 (m, 4H), 3.46 (m, 1H), 3.34 (m, 1H), 3.10-2.84 (m, 2H), 2.06-1.88 (m, 2H), 1.81 (m, 1H), 1.60 (m, 1H), 1.35 (d, J=6.8 Hz, 3H).

### Example 155

### Compound 1097

Step 1:4-(((R)-3-((R)-3-(dimethylamino)-2-methylenebutanamido)piperidin-1-yl)methyl)-N-(4-(4-morpholino-7H-pyrrolo[2,3-d]pyrimidin-6-yl)phenyl)picolinamide

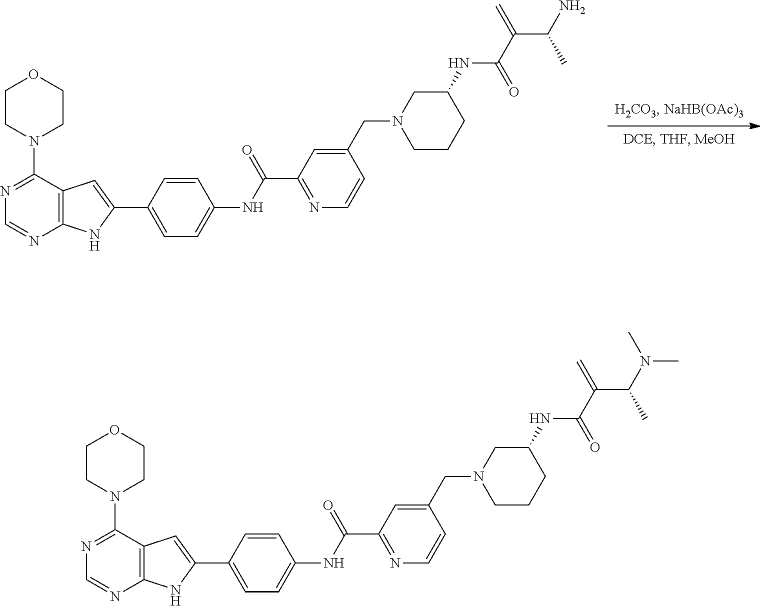 [SMILES: C=C(C(=O)N[C@@H]1CCCN(Cc2ccnc(C(=O)Nc3ccc(-c4cc5c(N6CCOCC6)ncnc5[nH]4)cc3)c2)C1)[C@@H](C)N ; "DCE, THF, MeOH" ; C=C(C(=O)N[C@@H]1CCCN(Cc2ccnc(C(=O)Nc3ccc(-c4cc5c(N6CCOCC6)ncnc5[nH]4)cc3)c2)C1)[C@@H](C)N(C)C]

A solution of 4-(((R)-3-((R)-3-amino-2-methylenebutanamido)piperidin-1-yl)methyl)-N-(4-(4-morpholino-7H-pyrrolo[2,3-d]pyrimidin-6-yl)phenyl)picolinamide trifluoroacetate (22 mg, 0.0304 mmol) in dichloroethane (1 mL), methanol (0.1 mL) and tetrahydrofuran (1 mL) was stirred at 21° C. for 5 minutes. Sodium triacetoxyborohydride (19 mg, 0.0912 mmol) was added. After 45 minutes the reaction was partitioned with ethyl acetate (20 mL) and saturated sodium bicarbonate (10 mL). The aqueous phase was extracted with ethyl acetate (3×10 mL). The combined organic phases were dried over sodium sulfate and the solvent was removed under reduced pressure. The residue was taken up in N,N-dimethylformamide (2 mL) and trifluoroacetic acid (4 drops). The solution was subjected to preparative HPLC(5-85% acetonitrile/water 0.1% trifluoroacetic acid). The fractions containing product were combined and subjected to lyophilization, providing 4-(((R)-3-((R)-3-(dimethylamino)-2-methylenebutanamido)piperidin-1-yl)methyl)-N-(4-(4-morpholino-7H-pyrrolo[2,3-d]pyrimidin-6-yl)phenyl)picolinamide trifluoroacetate (8.1 mg, 0.0108 mmol, 35%). LCMS=[M+H]⁺: 638.40, Purity 90%, ¹H NMR (400 MHz, MeOD) § 8.74 (dd, J=4.9, 0.8 Hz, 1H), 8.29 (d, J=1.6 Hz, 1H), 8.20 (s, 1H), 7.91-7.83 (m, 2H), 7.82-7.74 (m, 2H), 7.68 (dd, J=5.0, 1.8 Hz, 1H), 7.17 (s, 1H), 6.10 (s, 1H), 5.87 (s, 1H), 4.40 (m, 2H), 4.15 (m, 1H), 4.06-3.96 (m, 5H), 3.86-3.79 (m, 4H), 3.45 (m, 1H), 3.39-3.29 (m, 1H), 3.07-2.82 (m, 2H), 2.73 (s, 6H), 2.03-1.91 (m, 2H), 1.81 (m, 1H), 1.60 (m, 1H), 1.40 (d, J=6.8 Hz, 3H).

### Example 156

### Compound 1098

### Preparation of (R)-4-ethoxy-N-(1-(5-((4-(4-morpholino-7H-pyrrolo[2,3-d]pyrimidin-6-yl)phenyl)amino)pyrimidin-2-yl)piperidin-3-yl)but-2-ynamide (Compound 1098)

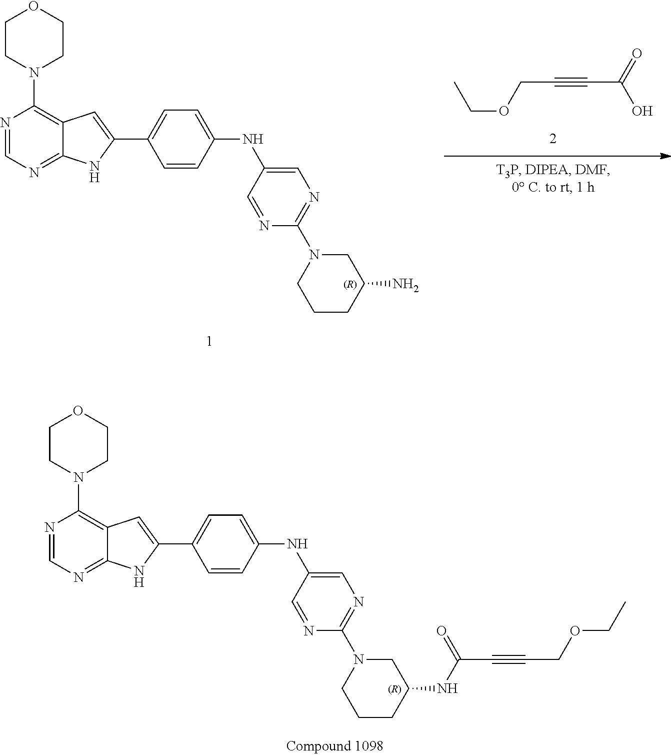

To a stirred solution of (R)-2-(3-aminopiperidin-1-yl)-N-(4-(4-morpholino-7H-pyrrolo[2,3-d]pyrimidin-6-yl)phenyl)pyrimidin-5-amine 1 (200 mg, 0.424 mmol) and 4-ethoxybut-2-ynoic acid 2 (110 mg, 0.848 mmol) in DMF (5 mL) was added DIPEA (0.25 mL, 1.27 mmol) and T3P (0.405 mL, 0.636 mmol; 50% solution in EtOAc) at 0° C. under N₂ atmosphere. The reaction mixture was stirred at RT for 1 h. After completion of reaction (monitored by TLC and LCMS), the reaction mixture was poured into ice cold water (50 mL) and extracted with EtOAc (3×70 mL). The combined organic layer was washed with ice cold brine (2×50 mL), dried over Na₂SO₄, filtered and concentrated under reduced pressure. The crude was purified by RP-HPLC [10-90% 5 mM Ammonium bicarbonate in water/ACN; Column: X Select CSH C18 (19*250) 10μ] to get Compound 1098 (8 mg, 3%).

¹H NMR (400 MHz, DMSO-d₆): δ 12.01 (s, 1H), 8.77 (d, J=8.0 Hz, 1H), 8.30 (s, 2H), 8.14 (s, 1H), 7.90 (s, 1H), 7.70 (d, J=8.4 Hz, 2H), 6.92 (d, J=2.0 Hz, 1H), 6.82 (d, J=8.8 Hz, 2H), 4.47-4.43 (m, 1H), 4.35-4.32 (m, 1H), 4.29 (s, 2H), 3.87-3.84 (m, 4H), 3.75-3.70 (m, 5H), 3.52 (q, J=6.8 Hz, 2H), 3.02-2.96 (m, 1H), 2.91-2.85 (m, 1H), 1.91-1.86 (m, 1H), 1.76-1.72 (m, 1H), 1.54-1.45 (m, 2H), 1.14 (t, J=6.8 Hz, 3H).

LCMS=[M+H]⁺: 582.46; Purity=97.23%.

### Synthesis of 4-ethoxybut-2-ynoic acid (2)

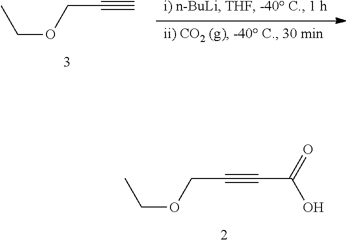

To a stirred solution of 3-ethoxyprop-1-yne 3 (1 g, 11.9 mmol) in dry THF (20 mL) was added n-BuLi (9.5 mL, 23.8 mmol) dropwise at −40° C. under N₂ atmosphere. The reaction mixture was stirred at −40° C. for 1h. Then CO₂ gas was purged into the reaction mixture for 30 min at same temperature. After completion of reaction (monitored by TLC and LCMS), the reaction mixture was diluted with water (50 mL) and extracted with Et20 (2×50 mL). The aqueous layer was acidified with conc. HCl to PH~4 and extracted with EtOAc (3×50 mL). The combined organic layer was washed with brine solution (2×30 mL), dried over Na₂SO₄, filtered and evaporated under reduced pressure to get the title compound as a brown oily liquid 2 (800 mg; crude), which was used in next step reaction without further purification.

¹H NMR (400 MHz, DMSO-d₆): δ 4.30 (s, 2H), 3.49 (q, J=6.8 Hz, 2H), 1.11 (t, J=7.2 Hz, 3H). [—CO₂H proton is not appeared]

### Example 157

### Compound 1099

### Preparation of (E)-4-(dimethylamino)-1-(8-(5-(hydroxy (4-(4-morpholino-7H-pyrrolo[2,3-d]pyrimidin-6-yl)phenyl)methyl)pyrimidin-2-yl)-3,8-diazabicyclo[3.2.1]octan-3-yl)but-2-en-1-one (Compound 1099)

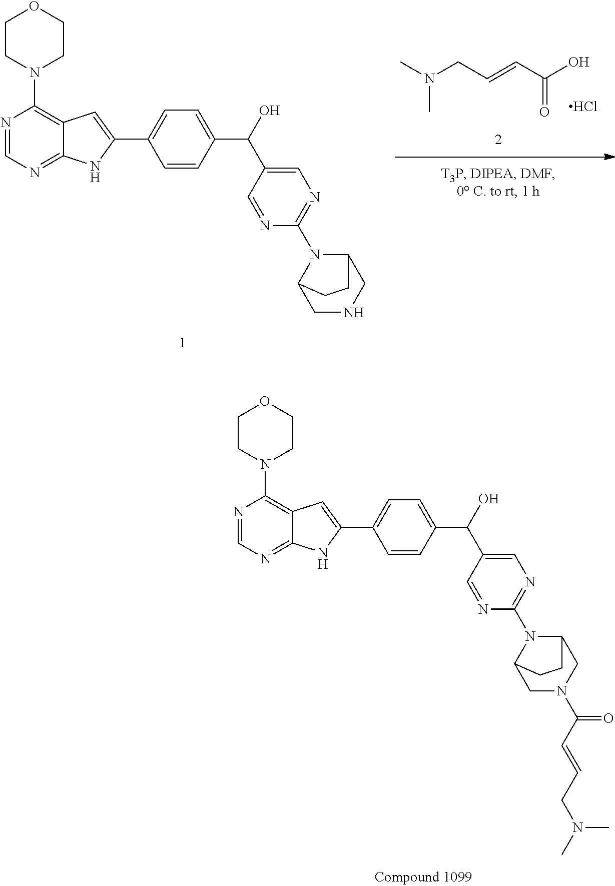

To a stirred solution of (2-(3,8-diazabicyclo[3.2.1]octan-8-yl)pyrimidin-5-yl)(4-(4-morpholino-7H-pyrrolo[2,3-d]pyrimidin-6-yl)phenyl)methanol 1 (200 mg, 0.401 mmol) and (E)-4-(dimethylamino)but-2-enoic acid hydrochloride 2 (100 mg, 0.602 mmol) in DMF (5 mL) was added DIPEA (0.2 mL, 1.2 mmol) and T3P (0.5 mL, 0.802 mmol; 50% solution in EtOAc) at 0° C. under N₂ atmosphere. The reaction mixture was stirred at RT for 1 h. After completion of reaction (monitored by TLC and LCMS), the reaction mixture was concentrated under reduced pressure. The crude was purified by RP-HPLC [2-98% 0.1% TFA in Water/ACN; Column: SunFire C18 (19*250) 10μ] to get Compound 1099 (22 mg, 9%).

¹H NMR (400 MHz, DMSO-d₆): δ 12.50 (s, 1H), 8.44 (s, 2H), 8.25 (s, 1H), 7.90 (d, J=8.4 Hz, 2H), 7.47 (d, J=8.0 Hz, 2H), 7.25 (s, 1H), 6.84 (d, J=15.2 Hz, 1H), 6.61-6.54 (m, 1H), 5.70 (s, 1H), 4.73 (brs, 2H), 4.23 (d, J=12.8 Hz, 1H), 3.92-3.90 (m, 4H), 3.84-3.81 (m, 4H), 3.78-3.76 (m, 4H), 3.33 (d, J=12.4 Hz, 1H), 2.85 (d, J=12.4 Hz, 1H), 2.77 (t, J=4.4 Hz, 6H), 1.90-1.85 (m, 2H), 1.68-1.62 (m, 2H).

LCMS=[M+H]⁺: 610.54; Purity=97.40%.

### Example 158

### Compound 1100

### Preparation of (R)-2-(azetidin-1-ylmethyl)-N-(3-methyl-1-(5-((4-(4-morpholino-7H-pyrrolo[2,3-d]pyrimidin-6-yl)phenyl)amino)pyrimidin-2-yl)piperidin-3-yl)acrylamide (Compound 1100)

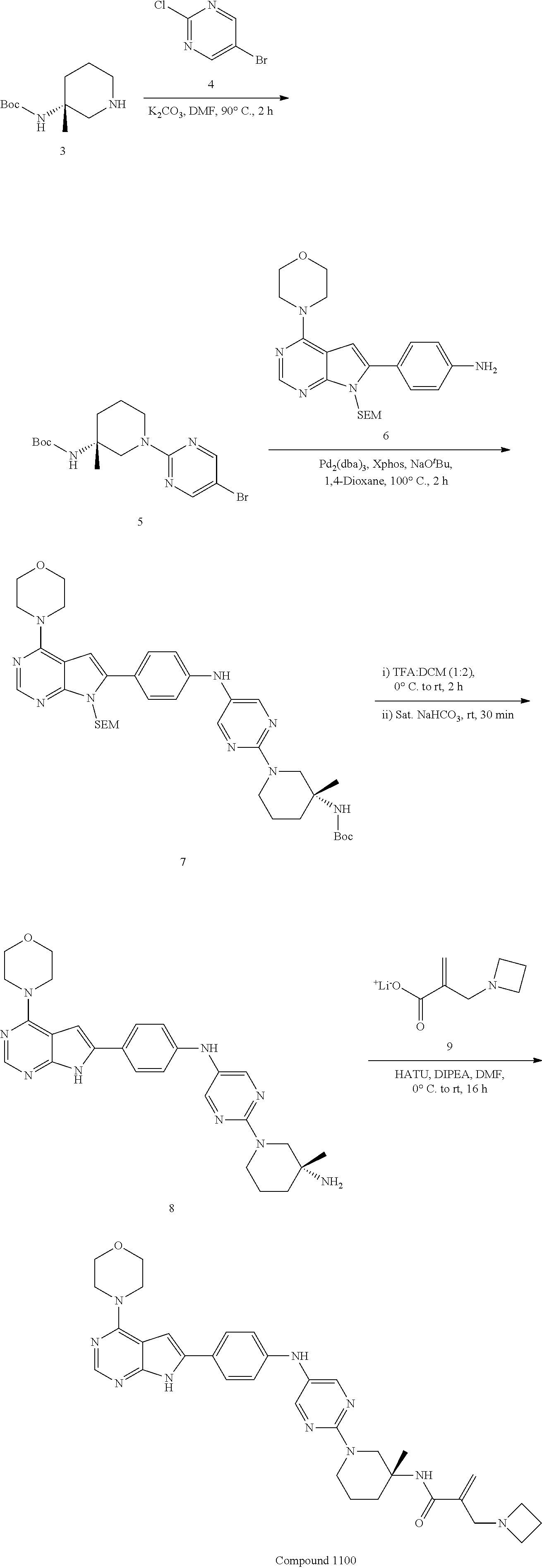

### Synthesis of tert-butyl (R)-(1-(5-bromopyrimidin-2-yl)-3-methylpiperidin-3-yl)carbamate (5)

To a stirred solution of tert-butyl (R)-(3-methylpiperidin-3-yl)carbamate 3 (400 mg, 1.87 mmol) in DMF (5 mL) was added 5-bromo-2-chloropyrimidine 4 (397 mg, 2.05 mmol) and K₂CO₃ (516 mg, 3.73 mmol) at RT under N₂ atmosphere. The reaction mixture was stirred at 90° C. for 2 h. After completion of reaction (monitored by TLC), the reaction mixture was diluted with ice cold water (50 mL) and extracted with EtOAc (3×50 mL). The combined organic layer was washed with ice cold brine (2×50 mL), dried over Na₂SO₄, filtered and evaporated under reduced pressure. The crude was purified by flash column chromatography (silica gel 230-400 M, 12 g SNAP) using eluents 20-30% EtOAc in heptane to get the title compound as a white solid 5 (600 mg, 86%).

LCMS=[M+H]⁺: 371.18; Purity=99%.

### Synthesis of tert-butyl (R)-(3-methyl-1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl)carbamate (7)

To a stirred solution of tert-butyl (R)-(1-(5-bromopyrimidin-2-yl)-3-methylpiperidin-3-yl)carbamate 5 (500 mg, 1.35 mmol) and 4-(4-morpholino-7-((2-(trimethylsilyl)ethoxy)methyl)-7H-pyrrolo[2,3-d]pyrimidin-6-yl)aniline 6 (688 mg, 1.62 mmol) in 1,4-Dioxane (10 mL) was added NaO^{t}Bu (194 mg, 2.02 mmol) at RT. The resulting reaction mixture was degassed with N₂ gas for 10 min, followed by added XPhos (128 mg, 0.269 mmol) and Pd₂(dba)₃ (109 mg, 0.135 mmol). The reaction mixture was stirred at 100° C. for 2 h. After completion of reaction (monitored by TLC and LCMS), the reaction mixture was diluted with water (60 mL) and extracted with EtOAc (3×70 mL). The combined organic layer was dried over Na₂SO₄, filtered and evaporated under reduced pressure. The crude was purified by flash column chromatography (silica gel 230-400 M, 12 g SNAP) using eluents 30-40% EtOAc in heptane to get the title compound as a yellow solid 7 (500 mg, 52%).

LCMS=[M+H]⁺: 716.56; Purity=72%.

### Synthesis of (R)-2-(3-amino-3-methylpiperidin-1-yl)-N-(4-(4-morpholino-7H-pyrrolo[2,3-d]pyrimidin-6-yl)phenyl)pyrimidin-5-amine (8)

To an ice cold stirred solution of tert-butyl (R)-(3-methyl-1-(5-((4-(4-morpholino-7-((2-(trimethylsilyl)ethoxy)methyl)-7H-pyrrolo[2,3-d]pyrimidin-6-yl)phenyl)amino)pyrimidin-2-yl)piperidin-3-yl)carbamate 7 (400 mg, 0.698 mmol) in DCM (6 mL) was added TFA (3 mL) dropwise under N₂ atmosphere. The reaction mixture was stirred at RT for 2 h. After completion of reaction (monitored by TLC and LCMS), the reaction mixture was concentrated under reduced pressure, triturated with Et₂O and dried under reduced pressure. The crude was dissolved in 25% IPA in CHCl₃ (5 mL), followed by added sat. NaHCO₃ (10 mL) and stirred for 30 min at RT. Then, the reaction mixture was extracted with 25% IPA in CHCl₃ (3×50 mL). The combined organic layer was dried over Na₂SO₄, filtered and concentrated under reduced pressure to get the title compound as a yellow solid 8 (300 mg; crude), which was used in next step directly without further purification.

LCMS=[M+H]⁺: 486.30; Purity=65%.

### Synthesis of (R)-2-(azetidin-1-ylmethyl)-N-(3-methyl-1-(5-((4-(4-morpholino-7H-pyrrolo[2,3-d]pyrimidin-6-yl)phenyl)amino)pyrimidin-2-yl)piperidin-3-yl)acrylamide (Compound 1100)

To an ice cold stirred solution of (R)-2-(3-amino-3-methylpiperidin-1-yl)-N-(4-(4-morpholino-7H-pyrrolo[2,3-d]pyrimidin-6-yl)phenyl)pyrimidin-5-amine 8 (200 mg, 0.412 mmol) in DMF (5 mL) was added lithium 2-(azetidin-1-ylmethyl)acrylate 9 (303 mg, 2.06 mmol), DIPEA (0.2 mL, 1.24 mmol) and HATU (235 mg, 0.618 mmol) sequentially under N₂ atmosphere. The resulting reaction mixture was stirred at RT for 16 h. After completion of reaction (monitored by TLC and LCMS), the reaction was diluted with ice cold water (50 mL) and extracted with 25% IPA in CHCl₃ (3×50 mL). The combined organic layer washed with ice cold brine (2×30 mL), dried over Na₂SO₄, filtered and concentrated under reduced pressure to get crude. The crude was purified by RP-HPLC [2-98% 0.1% TFA in Water and ACN; Column: SunFire C18 (19*250) 10μ] to get Compound 1100 (7 mg, 3%).

¹H NMR (400 MHz, DMSO-d₆): & 12.44 (s, 1H), 8.27 (s, 2H), 8.23 (s, 1H), 7.91 (brs, 1H), 7.71 (d, J=8.4 Hz, 2H), 7.62 (s, 1H), 7.04 (brs, 1H), 6.80 (d, J=8.8 Hz, 2H), 5.91 (s, 1H), 5.79 (s, 1H), 4.15-3.92 (m, 4H), 3.91-3.84 (m, 8H), 3.78-3.70 (m, 6H), 2.41-2.32 (m, 1H), 2.27-2.16 (m, 1H), 2.12-2.09 (m, 1H), 1.73-1.62 (m, 2H), 1.60-1.56 (m, 1H), 1.35 (s, 3H).

LCMS=[M+H]⁺: 609.53; Purity=96.90%.

### Example 159

### Compound 1101

### Preparation of 2-(azetidin-1-ylmethyl)-N-((3R)-1-(5-(hydroxy (4-(4-morpholino-7H-pyrrolo[2,3-d]pyrimidin-6-yl)phenyl)methyl)pyrimidin-2-yl)-3-methylpiperidin-3-yl)acrylamide (Compound 1101)

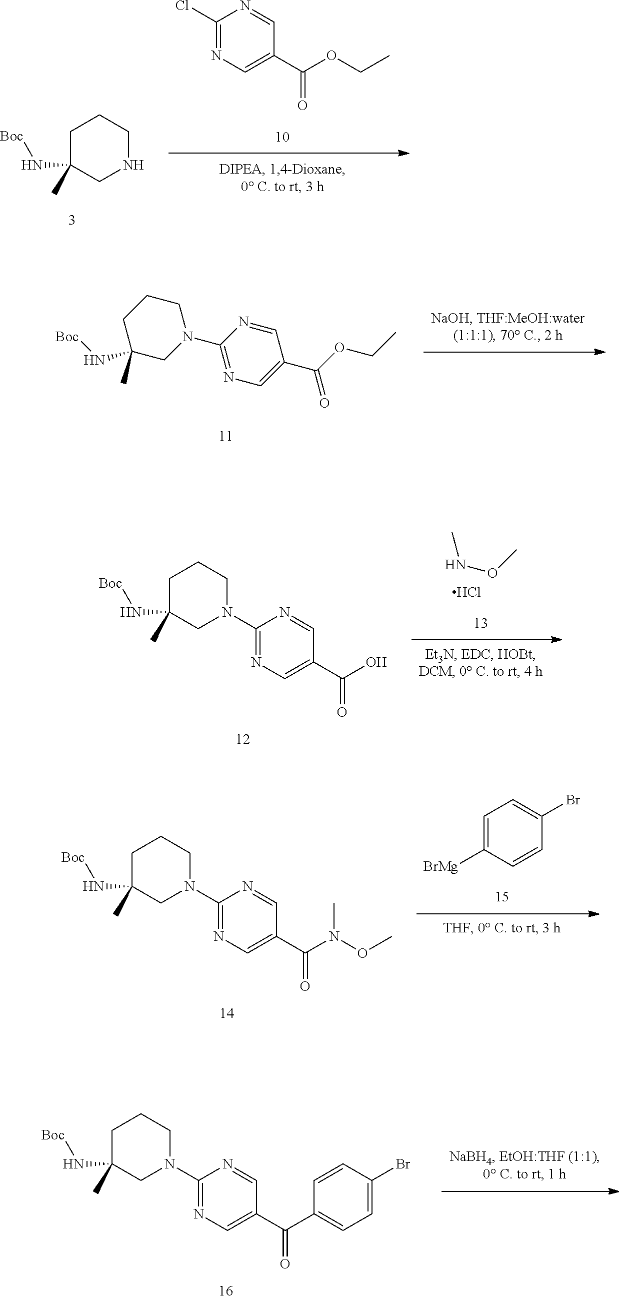

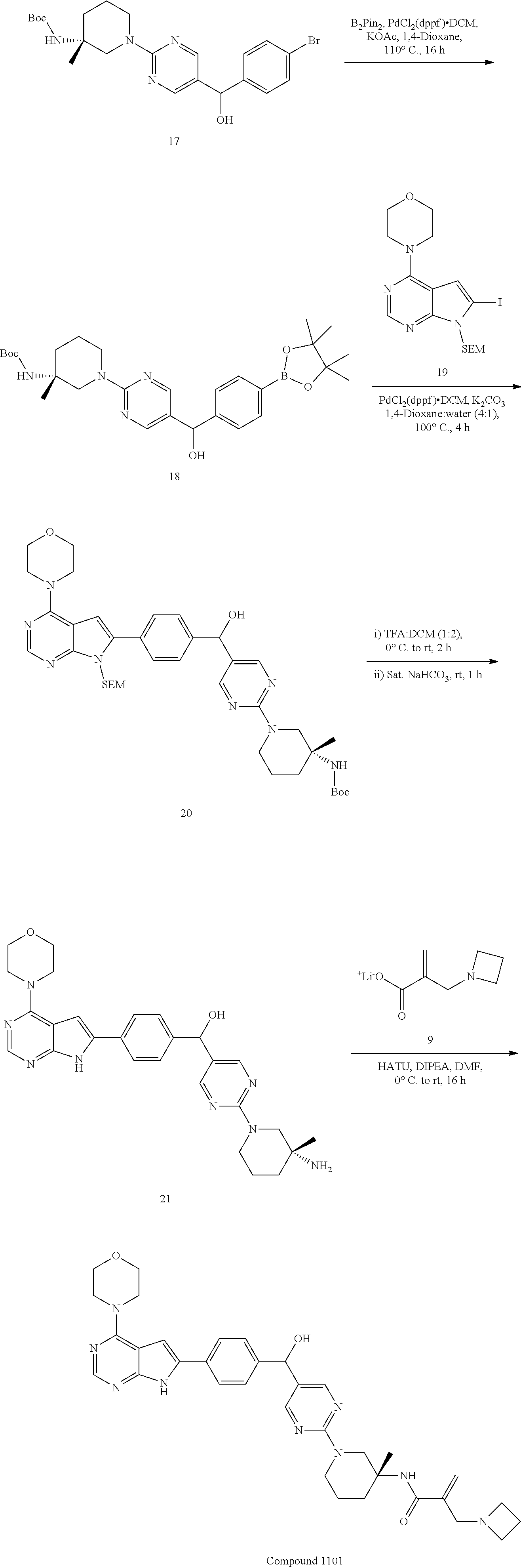

### Synthesis of ethyl (R)-2-(3-((tert-butoxycarbonyl)amino)-3-methylpiperidin-1-yl)pyrimidine-5-carboxylate (11)

To an ice cold stirred solution of tert-butyl (R)-(3-methylpiperidin-3-yl)carbamate 3 (1 g, 4.34 mmol) in 1,4-Dioxane (10 mL) was added DIPEA (2.3 mL, 13 mmol) dropwise under N₂ atmosphere. Then, ethyl 2-chloropyrimidine-5-carboxylate 10 (891 mg, 4.78 mmol) was added to the reaction mixture and stirred at RT for 3 h. After completion of reaction (monitored by TLC), the reaction mixture was concentrated under reduced pressure. The crude was purified by flash column chromatography (silica gel 230-400 M, 24 g SNAP) using eluents 25-30% EtOAc in heptane to get the title compound as pale yellow thick liquid 11 (1.3 g, 76%).

LCMS=[M+H]⁺: 365.20; Purity=92%.

### Synthesis of (R)-2-(3-((tert-butoxycarbonyl)amino)-3-methylpiperidin-1-yl)pyrimidine-5-carboxylic acid (12)

To a stirred solution of ethyl (R)-2-(3-((tert-butoxycarbonyl)amino)-3-methylpiperidin-1-yl)pyrimidine-5-carboxylate 11 (1.3 g, 3.57 mmol) in THF (10 mL) and MeOH (10 mL) was added an aqueous solution of NaOH (285 mg, 7.13 mmol) dissolved in water (10 mL) at RT. The resulting mixture was stirred at 70° C. for 2 h. After completion of reaction (monitored by TLC), the reaction mixture was concentrated under reduced pressure. The crude was diluted with water (30 mL) and acidified with 1N HCl to pH~5. The obtained precipitate was filtered through sintered funnel, washed with Et₂O and dried under reduced pressure to get the title compound as a white solid 12 (940 mg, 79%).

¹H NMR (400 MHz, DMSO-d₆): δ 12.79 (s, 1H), 8.71 (s, 2H), 6.41 (s, 1H), 4.21-4.28 (m, 1H), 3.83-3.72 (m, 3H), 1.87-1.83 (m, 1H), 1.69-1.50 (m, 3H), 1.49 (s, 9H), 1.21 (s, 3H).

LCMS=[M+H]⁺: 337.26; Purity=99%.

### Synthesis of tert-butyl (R)-(1-(5-(methoxy(methyl)carbamoyl)pyrimidin-2-yl)-3-methylpiperidin-3-yl) carbamate (14)

To an ice cold stirred solution of (R)-2-(3-((tert-butoxycarbonyl)amino)-3-methylpiperidin-1-yl)pyrimidine-5-carboxylic acid 12 (940 mg, 2.79 mmol) in DCM (20 mL) was added Et₃N (1.6 mL, 11.2 mmol) under N₂ atmosphere. Then, EDC·HCl (804 mg, 4.19 mmol) and HOBT (453 mg, 3.35 mmol) were added to the reaction mixture. The resulting reaction mixture was stirred at RT for 1 h. Then, the reaction mixture was cooled to 0° C. and N,O-dimethylhydroxylamine hydrochloride 13 (327 mg, 3.35 mmol) was added and stirred at RT for 4 h. After completion of reaction (monitored by TLC and LCMS), the reaction mixture was diluted with water (50 mL) and extracted with DCM (3×70 mL). The combined organic layer was dried over Na₂SO₄, filtered and concentrated under reduced pressure. The crude was purified by flash column chromatography (silica gel 230-400 M, 24 g SNAP) using eluents 15-20% EtOAc in heptane to get the title compound as a white solid 14 (1 g, 94%).

LCMS=[M+H]⁺: 380.25; Purity=98%.

### Synthesis of tert-butyl (R)-(1-(5-(4-bromobenzoyl)pyrimidin-2-yl)-3-methylpiperidin-3-yl)carbamate (16)

To an ice cold stirred solution of tert-butyl (R)-(1-(5-(methoxy(methyl)carbamoyl)pyrimidin-2-yl)-3-methylpiperidin-3-yl)carbamate 14 (1 g, 2.64 mmol) in dry THF (40 mL) was added (4-bromophenyl) magnesium bromide 15 (21 mL, 10.5 mmol; 0.5 M in THF) dropwise under N₂ atmosphere. The resulting reaction mixture was stirred at RT for 3 h. After completion of reaction (monitored by TLC), the reaction mixture was quenched with sat. NH₄Cl solution (50 mL) and extracted with EtOAc (3×80 mL). The combined organic layer was dried over Na₂SO₄, filtered and concentrated under reduced pressure. The crude was purified by flash column chromatography (silica gel 230-400 M, 24 g SNAP) using eluents 15-18% EtOAc in heptane to get the title compound as a colorless sticky solid 16 (750 mg, 60%).

LCMS=[M+H]⁺: 475.16; Purity=88%.

### Synthesis of tert-butyl ((3R)-1-(5-((4-bromophenyl)(hydroxy)methyl)pyrimidin-2-yl)-3-methylpiperidin-3-yl) carbamate (17)

To an ice cold stirred solution of tert-butyl (R)-(1-(5-(4-bromobenzoyl)pyrimidin-2-yl)-3-methylpiperidin-3-yl)carbamate 16 (700 mg, 1.47 mmol) in EtOH (5 mL) and THF (5 mL) was added NaBH₄ (111 mg, 2.94 mmol) under N₂ atmosphere. The resulting reaction mixture was stirred at RT for 1 h. After completion of reaction (monitored by TLC), the reaction mixture was quenched with sat. NH₄Cl solution (50 mL). The reaction mixture was concentrated under reduced pressure. The crude was diluted with H₂O (30 mL) and extracted with EtOAc (3×70 mL). The combined organic layer was dried over Na₂SO₄, filtered and concentrated under reduced pressure to get the title compound as a white solid 17 (690 mg, 98%), which was used in next step directly without further purification.

LCMS=[M+H]⁺: 477.18; Purity=88%.

### Synthesis of tert-butyl ((3R)-1-(5-(hydroxy (4-(4,4,5,5-tetramethyl-1,3,2-dioxaborolan-2-yl)phenyl)methyl)pyrimidin-2-yl)-3-methylpiperidin-3-yl)carbamate (18)

To a stirred solution of tert-butyl ((3R)-1-(5-((4-bromophenyl)(hydroxy)methyl)pyrimidin-2-yl)-3-methylpiperidin-3-yl)carbamate 17 (690 mg, 1.45 mmol) in 1,4-Dioxane (10 mL) were added KOAc (426 mg, 4.34 mmol) and B₂Pin₂ (440 mg, 1.73 mmol) at RT under N₂ atmosphere. The reaction mixture was purged with N2 gas for 10 min, followed by added PdCl₂(dppf)·DCM (118 mg, 0.145 mmol). The reaction mixture was stirred at 100° C. for 16 h. After completion of reaction (monitored by LCMS), the reaction mixture was diluted with EtOAc (80 mL) and filtered through celite bed. The filtrate was washed with water (2×30 mL). The organic layer was dried over Na₂SO₄, filtered and concentrated under reduced pressure to get the title compound as a brown sticky solid 18 (755 mg; crude), which was used in next reaction without further purification.

LCMS=[M+H]⁺: 525.35; Purity=60%.

### Synthesis of tert-butyl ((3R)-1-(5-(hydroxy (4-(4-morpholino-7-((2-(trimethylsilyl)ethoxy)methyl)-7H-pyrrolo[2,3-d]pyrimidin-6-yl)phenyl)methyl)pyrimidin-2-yl)-3-methylpiperidin-3-yl)carbamate (20)

To a stirred solution of 4-(6-iodo-7-((2-(trimethylsilyl)ethoxy)methyl)-7H-pyrrolo[2,3-d]pyrimidin-4-yl)morpholine 19 (500 mg, 1.09 mmol) in 1,4-Dioxane (8 mL) was added tert-butyl ((3R)-1-(5-(hydroxy (4-(4,4,5,5-tetramethyl-1,3,2-dioxaborolan-2-yl)phenyl)methyl)pyrimidin-2-yl)-3-methylpiperidin-3-yl)carbamate 18 (854 mg, 1.33 mmol) and K₂CO₃ (450 mg, 3.26 mmol) dissolved in water (2 mL) at RT under N2 atmosphere. The reaction mixture was purged with N₂ gas for 10 min, followed by added PdCl₂(dppf)·DCM (88 mg, 0.109 mmol). The resulting reaction was stirred at 100° C. for 4 h. After completion of reaction (monitored by TLC and LCMS), the reaction mixture was diluted with water (60 mL) and extracted with EtOAc (3×80 mL). The combined organic layer was dried over Na₂SO₄, filtered and concentrated under reduced pressure. The crude was purified by flash column chromatography (silica gel 230-400 M, 12 g SNAP) using eluents 50-60% EtOAc in heptane to get the title compound as a white solid 20 (510 mg, 64%).

LCMS=[M+H]⁺: 731.54; Purity=87.37%.

### Synthesis of (2-((R)-3-amino-3-methylpiperidin-1-yl)pyrimidin-5-yl)(4-(4-morpholino-7H-pyrrolo[2,3-d]pyrimidin-6-yl)phenyl)methanol (21)

To a stirred solution of tert-butyl ((3R)-1-(5-(hydroxy (4-(4-morpholino-7-((2-(trimethylsilyl)ethoxy)methyl)-7H-pyrrolo[2,3-d]pyrimidin-6-yl)phenyl)methyl)pyrimidin-2-yl)-3-methylpiperidin-3-yl)carbamate 20 (500 mg, 0.684 mmol) in DCM (8 mL) was added TFA (4 mL) dropwise at 0° C. under N₂ atmosphere. The resulting reaction mixture was stirred at RT for 2 h. After completion of reaction (monitored by TLC and LCMS), the reaction mixture was concentrated under reduced pressure. The crude was dissolved in 25% IPA in CHCl₃ (5 mL), followed by added sat. NaHCO₃ solution (10 mL) and stirred at RT for 1 h. The resulting mixture was further diluted with water (20 mL) and extracted with 25% IPA in CHCl₃ (3×40 mL). The combined organic layer were dried over Na₂SO₄, filtered and concentrated under reduced pressure to get the title compound as a light yellow solid 21 (340 mg; crude), which was used in next step reaction directly without further purification.

LCMS=[M+H]⁺: 501.34; Purity=78%.

### Synthesis of 2-(azetidin-1-ylmethyl)-N-((3R)-1-(5-(hydroxy (4-(4-morpholino-7H-pyrrolo[2,3-d]pyrimidin-6-yl)phenyl)methyl)pyrimidin-2-yl)-3-methylpiperidin-3-yl)acrylamide (Compound 1101)

To an ice cold stirred solution of (2-((R)-3-amino-3-methylpiperidin-1-yl)pyrimidin-5-yl)(4-(4-morpholino-7H-pyrrolo[2,3-d]pyrimidin-6-yl)phenyl)methanol 21 (200 mg, 0.4 mmol) in DMF (5 mL) was added lithium 2-(azetidin-1-ylmethyl)acrylate 9 (294 mg, 2 mmol), DIPEA (0.2 mL, 1.2 mmol) and HATU (228 mg, 0.599 mmol) sequentially at RT under N₂ atmosphere. The reaction mixture was stirred at RT for 16 h. After completion of reaction (monitored by TLC and LCMS), the reaction mixture was poured into ice cold water (20 mL) and extracted with 25% IPA in CHCl₃ (3×40 mL). The combined organic layer was washed with chilled brine (2×30 mL), dried over Na₂SO₄, filtered and concentrated under reduced pressure. The crude was purified by RP-HPLC [2-98% 0.1% TFA in water/ACN; Column: SunFire C18 (19*250) 10μ] to get Compound 1101 (19 mg, 8%).

¹H NMR (400 MHz, DMSO-d₆): δ 12.43 (s, 1H), 8.27 (d, J=2.0 Hz, 2H), 8.23 (s, 1H), 7.88 (d, J=8.4 Hz, 2H), 7.57 (s, 1H), 7.43 (d, J=7.6 Hz, 2H), 7.23 (brs, 1H), 5.79 (d, J=8.8 Hz, 1H), 5.70 (d, J=7.2 Hz, 1H), 5.64 (s, 1H), 4.07 (t, J=12.4 Hz, 2H), 3.96-3.81 (m, 10H), 3.79-3.77 (m, 4H), 3.75-3.64 (m, 2H), 2.34-2.30 (m, 2H), 2.27-2.12 (m, 2H), 1.68-1.62 (m, 2H), 1.53-1.51 (m, 1H), 1.29 (s, 3H).

LCMS=[M+H]⁺: 624.56; Purity=96.95%.

### Example 160

### Compound 1102

### Preparation of (E)-4-(dimethylamino)-1-(8-(5-((4-(4-morpholino-7H-pyrrolo[2,3-d]pyrimidin-6-yl)phenyl)amino)pyrimidin-2-yl)-3,8-diazabicyclo[3.2.1]octan-3-yl)but-2-en-1-one (Compound 1102)

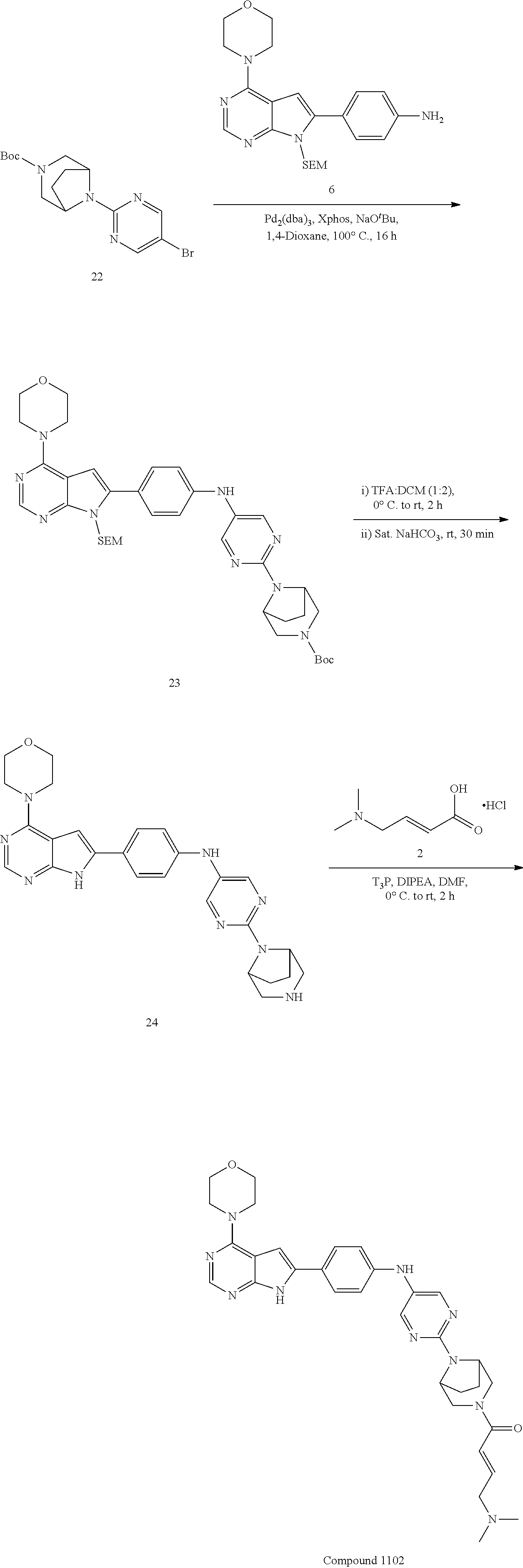

### Synthesis of tert-butyl 8-(5-((4-(4-morpholino-7-((2-(trimethylsilyl)ethoxy)methyl)-7H-pyrrolo[2,3-d]pyrimidin-6-yl)phenyl)amino)pyrimidin-2-yl)-3,8-diazabicyclo[3.2.1]octane-3-carboxylate (23)

To a stirred solution of tert-butyl 8-(5-bromopyrimidin-2-yl)-3,8-diazabicyclo[3.2.1]octane-3-carboxylate 22 (1 g, 2.71 mmol) and 4-(4-morpholino-7-((2-(trimethylsilyl)ethoxy)methyl)-7H-pyrrolo[2,3-d]pyrimidin-6-yl)aniline 6 (1.38 g, 3.25 mmol) in 1,4-Dioxane (10 mL) was added NaO^{t}Bu (390 mg, 4.06 mmol) at RT. The resulting reaction mixture was degassed with N₂ gas for 10 min, followed by added Pd₂dba₃ (248 mg, 0.271 mmol) and XPhos (129 mg, 0.271 mmol). The resulting reaction mixture was stirred at 100° C. for 16 h. After completion of reaction (monitored by TLC and LCMS), the reaction mixture was diluted with water (80 mL) and extracted with EtOAc (3×80 mL). The combined organic layer was dried over Na₂SO₄, filtered and evaporated under reduced pressure. The crude was purified by flash chromatography (silica gel 100-200 M, 40 g SNAP) using eluents 15-20% EtOAc in heptane to get the title compound as a yellow solid 23 (1.5 g, 74%).

LCMS=[M+H]⁺: 714.53; Purity=97%.

### Synthesis of 2-(3,8-diazabicyclo[3.2.1]octan-8-yl)-N-(4-(4-morpholino-7H-pyrrolo[2,3-d]pyrimidin-6-yl)phenyl)pyrimidin-5-amine (24)

To an ice cold stirred solution of tert-butyl 8-(5-((4-(4-morpholino-7-((2-(trimethylsilyl)ethoxy)methyl)-7H-pyrrolo[2,3-d]pyrimidin-6-yl)phenyl)amino)pyrimidin-2-yl)-3,8-diazabicyclo[3.2.1]octane-3-carboxylate 23 (1.5 g, 2.1 mmol) in DCM (16 mL) was added a solution of TFA (8 mL, 103 mmol) in DCM (10 mL) at 0° C. and stirred the reaction mixture at RT for 2 h. After completion of reaction (monitored by TLC and LCMS), the reaction mixture was concentrated under reduced pressure. The crude was quenched with NaHCO₃ and stirred for 30 min at RT. Then, the reaction mixture was extracted with 25% IPA in CHCl₃ (3×80 mL). The combined organic layer was dried over Na₂SO₄, filtered and concentrate under reduced pressure to get the title compound as a yellow solid 24 (1 g; crude), which was directly used in next step reaction without any purification.

LCMS=[M+H]⁺: 484.28; Purity=85%

### Synthesis of (E)-4-(dimethylamino)-1-(8-(5-((4-(4-morpholino-7H-pyrrolo[2,3-d]pyrimidin-6-yl)phenyl)amino)pyrimidin-2-yl)-3,8-diazabicyclo[3.2.1]octan-3-yl)but-2-en-1-one (Compound 1102)

To an ice cold stirred solution of 2-(3,8-diazabicyclo[3.2.1]octan-8-yl)-N-(4-(4-morpholino-7H-pyrrolo[2,3-d]pyrimidin-6-yl)phenyl)pyrimidin-5-amine 24 (300 mg, 0.620 mmol) and (E)-4-(dimethylamino)but-2-enoic acid hydrochloride 2 (154 mg, 0.931 mmol) in DMF (5 mL) were added DIPEA (0.56 mL, 3.1 mmol) and T3P (0.6 mL, 0.931 mmol; 50% solution in EtOAc) at 0° C. under N₂ atmosphere. The resulting reaction mixture was stirred at RT for 2 h. After completion of reaction (monitored by TLC and LCMS), the reaction mixture was poured into ice cold water (20 mL) and extracted with 25% IPA in CHCl₃ (3×40 mL). The combined organic layer was dried over Na₂SO₄, filtered and evaporated under reduced pressure. The crude was purified by RP-HPLC [2-98% 0.1% TFA in water/ACN; Column: SunFire C18 (19*250) 10μ] to get Compound 1102 (36 mg, 8%).

¹H NMR (400 MHz, DMSO-d₆): δ 12.47 (s, 1H), 8.37 (s, 2H), 8.24 (s, 1H), 7.73 (d, J=8.8 Hz, 2H), 7.07 (s, 1H), 6.96-6.85 (m, 3H), 6.63-6.56 (m, 1H), 4.72 (brs, 2H), 4.24 (d, J=13.2 Hz, 1H), 3.91-3.83 (m, 8H), 3.78-3.76 (m, 4H), 3.41 (d, J=12.0 Hz, 1H), 2.94 (d, J=12.4 Hz, 1H), 2.78 (t, J=4.8 Hz, 6H), 1.93-1.89 (m, 2H), 1.71-1.60 (m, 2H).

LCMS=[M+H]⁺: 595.54; Purity=98.12%.

### Example 161

### Compound 1103

### Preparation of 1-(4-(5-(2,2,2-trifluoro-1-hydroxy-1-(4-(4-morpholino-7H-pyrrolo[2,3-d]pyrimidin-6-yl)phenyl)ethyl)pyrimidin-2-yl)piperazin-1-yl)prop-2-en-1-one (Compound 1103)

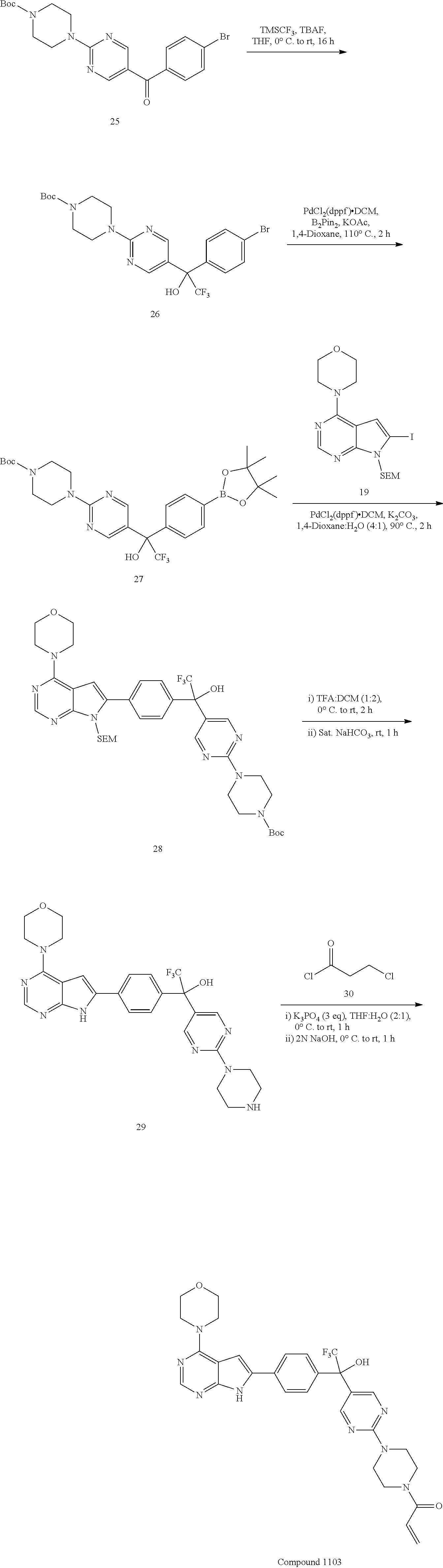

### Synthesis of tert-butyl 4-(5-(1-(4-bromophenyl)-2,2,2-trifluoro-1-hydroxyethyl)pyrimidin-2-yl)piperazine-1-carboxylate (26)

To a stirred solution of tert-butyl 4-(5-(4-bromobenzoyl)pyrimidin-2-yl)piperazine-1-carboxylate 25 (500 mg, 1.12 mmol) in dry THF (10 mL) was added TMSCF₃ (0.3 mL, 2.24 mmol) at 0° C. under N₂ atmosphere and stirred for 5 min. Then, TBAF (1.12 mL, 1.12 mmol; IM in THF) was added to the reaction mixture at 0° C. and stirred at RT for 16 h. After completion of reaction (monitored by TLC and LCMS), the reaction mixture was diluted with water (50 mL) and extracted with EtOAc (3×60 mL). The combined organic layer was dried over Na₂SO₄, filtered and concentrated under reduced pressure. The crude was purified by flash column chromatography (silica gel 230-400 M, 12 g SNAP) using eluents 30-35% EtOAc in heptane to get the title compound as a light white solid 26 (400 mg, 69%).

LCMS=[M+H]⁺: 517.13; Purity=83%.

### Synthesis of tert-butyl 4-(5-(2,2,2-trifluoro-1-hydroxy-1-(4-(4,4,5,5-tetramethyl-1,3,2-dioxaborolan-2-yl)phenyl)ethyl)pyrimidin-2-yl)piperazine-1-carboxylate (27)

To a stirred solution of tert-butyl 4-(5-(1-(4-bromophenyl)-2,2,2-trifluoro-1-hydroxyethyl)pyrimidin-2-yl)piperazine-1-carboxylate 26 (400 mg, 0.773 mmol) and B₂Pin₂ (295 mg, 1.16 mmol) in 1,4-Dioxane (10 mL) was added KOAc (228 mg, 2.32 mmol). The resulting reaction mixture was degassed with N₂ gas for 10 min, followed by added PdCl₂(dppf)·DCM (63 mg, 0.077 mmol). The reaction mixture was stirred at 110° C. for 2 h. After completion of reaction (monitored by TLC and LCMS), the reaction mixture was filtered through celite bed and washed with EtOAc (50 mL). The filtrate was evaporated under reduced pressure to get the title compound as brown sticky oil 27 (450 mg; crude), which was used in next step directly without further purification.

LCMS=[M+H]⁺: 565.29; Purity=65%.

### Synthesis of tert-butyl 4-(5-(4-bromobenzoyl)pyrimidin-2-yl)piperazine-1-carboxylate (28)

To a stirred solution of tert-butyl 4-(5-(2,2,2-trifluoro-1-hydroxy-1-(4-(4,4,5,5-tetramethyl-1,3,2-dioxaborolan-2-yl)phenyl)ethyl)pyrimidin-2-yl)piperazine-1-carboxylate 27 (442 mg, 0.782 mmol) and 4-(6-iodo-7-((2-(trimethylsilyl)ethoxy)methyl)-7H-pyrrolo[2,3-d]pyrimidin-4-yl)morpholine 19 (300 mg, 0.652 mmol) in 1,4-dioxane (8 mL) and water (2 mL) was added K₂CO₃ (270 mg, 0.652 mmol) at RT. The resulting reaction mixture was degassed with N₂ gas for 15 min, followed by added PdCl₂(dppf)·DCM (53.2 mg, 0.065 mmol). The reaction mixture was stirred at 90° C. for 2 h. After completion of reaction (monitored by TLC), the reaction mixture was diluted with water (30 mL) and extracted with EtOAc (3×50 mL). The combined organic layer was washed with brine solution (2×0 mL), dried over Na₂SO₄, filtered and evaporated under reduced pressure. The crude was purified by flash column chromatography (silica gel 230-400 M, 12 g SNAP) using eluents 30-40% EtOAc in heptane to get the title compound as a yellow solid 28 (450 mg, 89%).

LCMS=[M+H]⁺: 771.57; Purity=70%

### Synthesis of 2,2,2-trifluoro-1-(4-(4-morpholino-7H-pyrrolo[2,3-d]pyrimidin-6-yl)phenyl)-1-(2-(piperazin-1-yl)pyrimidin-5-yl) ethan-1-ol (29)

To an ice cold stirred solution of tert-butyl 4-(5-(4-bromobenzoyl)pyrimidin-2-yl)piperazine-1-carboxylate 28 (400 mg, 0.276 mmol) in DCM (4 mL) was added TFA (2 mL) dropwise at 0° C. under N₂ atmosphere. The reaction mixture was stirred at RT for 2 h. After completion of reaction (monitored by TLC and LCMS), the reaction mixture was concentrated under reduced pressure. The obtained crude was dissolved in 25% IPA in CHCl₃ (6 mL) and followed by added saturated aq. NaHCO₃ (6 mL). The reaction mixture was stirred at RT for 1 h. After completion of reaction (monitored by LCMS), the reaction was diluted with water (50 mL) and extracted with 25% IPA in CHCl₃ (3×50 mL). The combined organic layer was dried over Na₂SO₄, filtered and concentrated under reduced pressure to get the title compound as a brown solid 29 (180 mg; crude), which was used in next step directly without further purification.

LCMS=[M+H]⁺: 541.23; Purity=75%.

### Synthesis of 1-(4-(5-(2,2,2-trifluoro-1-hydroxy-1-(4-(4-morpholino-7H-pyrrolo[2,3-d]pyrimidin-6-yl)phenyl)ethyl)pyrimidin-2-yl)piperazin-1-yl)prop-2-en-1-one (Compound 1103)

To an ice cold stirred solution of 2,2,2-trifluoro-1-(4-(4-morpholino-7H-pyrrolo[2,3-d]pyrimidin-6-yl)phenyl)-1-(2-(piperazin-1-yl)pyrimidin-5-yl) ethan-1-ol 29 (180 mg, 0.33 mmol) in THF (4 mL) was added K₃PO₄ (212 mg, 0.99 mmol) dissolved with water (2 mL) dropwise under N₂ atmosphere. Then, a solution of 3-chloropropanoyl chloride 30 (51 mg, 0.40 mmol) dissolved in THF (0.5 mL) was added dropwise. The resulting reaction mixture was stirred at RT for 1 h. After completion of starting material (acylation reaction, monitored by TLC), 2N aq. NaOH (2 mL, 0.333 mmol) was added to the reaction mixture at 0° C. and stirred at RT for 1 h. After completion of reaction (complete elimination, monitored by TLC and LCMS), the reaction mixture was diluted with water (20 mL) and extracted with 2-MeTHF (3×50 mL). The combined organic layer was dried over Na₂SO₄, filtered and concentrated under reduced pressure. The crude residue was purified by flash column chromatography (silica gel 230-400 M, 4 g SNAP) using eluents 3-4% MeOH in DCM to get Compound 1103 (7 mg, 3.5%).

¹H NMR (400 MHz, DMSO-d₆): δ 12.31 (s, 1H), 8.34 (s, 2H), 8.19 (s, 1H), 7.95 (d, J=8.0 Hz, 2H), 7.52-7.50 (m, 3H), 7.26 (s, 1H), 6.83 (q, J=10.4 Hz, 1H), 6.14 (d, J=16.8 Hz, 1H), 5.71 (d, J=10.0 Hz, 1H), 3.90-3.82 (m, 4H), 3.79-3.73 (m, 8H), 3.65-3.62 (m, 4H).

LCMS=[M+H]⁺: 595.36; Purity=98.17%.

### Additional Exemplary Compounds

Other compounds provided herein have been or can be prepared according to the synthetic methods, or some variations thereof, described herein. The compounds can be prepared from readily available starting materials using the general methods and procedures described herein or described in U.S. Pat. No. 11,084,825 or in PCT/US2021/063671 (incorporated by reference in their entireties). It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The representative compounds prepared or can be prepared from readily available starting materials using the general methods and procedures described herein or described in U.S. Pat. No. 11,084,825 or in PCT/US2021/063671 (incorporated by reference in their entireties) are depicted in Table 1.

### Example B1a

### In Vitro Inhibitory Activity

The present example evaluates the ability of compounds described herein, inhibitors of menin or menin/MLL interaction, to inhibit cell proliferation. The proliferation inhibitory effect was investigated in human MLL-leukemia cells selected on the bases of MLL fusion proteins and listed in the table below.

| Cell | MLL gene | | Cat. |
|---|---|---|---|
| Type | fusion | Supplier/Vendor | Number |
| K-562 | BCR-ABL | ATCC | CCL-243 |
| MV4-11 | MLL-AF4 | ATCC/LCC | CRL-9591 |
| MOLM-13 | MLL-AF9 | AddexBio/DBA | C003003 |
| OCI-AML3 | AML | ATCC | CRL-1593.2 |
| U-937 | MLL-wt | Creative Bioarray | CSC-C9119W |

ATP is present in all metabolically active cells and is considered as a marker for cell viability and proliferation. The metabolic cell activity was determined using the CellTiter-Glo kit from Promega, an ATP monitoring system based on the production of luminescence by the reaction of ATP with added UltraGlo® recombinant luciferase (Kawano et al., 2016, PLOS One, 8: 11 (7): e0158888), according to the supplier's experimental recommendations. The assay is based on a 96 well plate format. All experiments were carried out in duplicate in one test occasion.

Test items were dissolved at 20 mM in DMSO with purity ≥99.9% (Sigma catalog no. D8418) and stored at −20° C. Cells were maintained in RPMI-1640 medium (Invitrogen catalog no. 618700) supplemented with 10% of Heat Inactivated FBS (Invitrogen catalog no. 10500) and 1% Pen-Strep (Invitrogen catalog no. 15140) and cultured at 37° C. in a humidified incubator with 5% CO₂. Cell lines were grown in suspension and the cell density was maintained in a range of 5×10⁴-1×10⁶ viable cells/ml where possible. Cells were pelleted at 130 g×5 min.

Serial dilutions of compounds 1 to 3 in DMSO 100% were prepared starting from a 2.5 mM solutions (prepared by diluting 1:8 the 20 mM stock solution) to generate a 7 point concentration response curve (CRC). Seven concentrations of test compounds (e.g., 5.00×10⁻⁶-1.67×10⁻⁶-5.56×10⁻⁷-1.85×10⁻⁷-6.17×10⁻⁸-2.06×10⁻⁸-6.86×10⁻⁹ M) were assessed in duplicate in an individual test occasion in parallel in the selected cell lines. KO-539 was used as a reference compound and tested in duplicate at seven similar concentrations. 100% of proliferation was represented by the untreated cells (0.2% DMSO).

Cell proliferation inhibition was monitored at both the following end points: Day 4 (T4) and Day 7 (T7). For the appropriate cell density during the exponential cell growth and re-supply with fresh compound solution at T4, cells were diluted (1:4) with fresh medium containing the compound concentration (1X).

For each plate to test, one 0.4 μL copy plate and four 0.3 μL copy plates were stamped into 96-well plates not treated for cell adhesion (Sarstedt-cat. no. 82.1581.001) by acoustic liquid handling (Echo) at a concentration which was 500 fold the final assay concentration. Stamped plates were stored at −20° C.

On Day 0, the day of the experiment cell line suspensions were counted by Cell Viability Analyser, Vi-CELL (Beckman Coulter AY15292), and diluted with fresh medium to obtain the following cell densities: K-562 500 c/ml, MOLM-13 1,000 c/ml, MV4-11 10,000 c/ml, OCI-AML-3 500 c/ml, and U-937 500-1,000 c/ml. 200 μL/well and 150 μL/well of cell suspension were added into the 0.4 μL/well and 0.3 μL/well compound plates, respectively. Cell plate containing 200 μL/well suspension were incubated at 37° C. in a humidified incubator with 5% CO₂. From each well of the 150 μL/well cell assay plate, 100 μL were harvested and transferred into a 96-well Optiplate (Perkin Elmer, cat. no. 6005290). Cell viability was measured as described below.

On Day 4, 150 μL/well of fresh medium was added into a new 0.3 μL/well copy compound plates. From each well of the 200 μL/well cell assay plate, 100 μL was sampled for the cell viability measurement as described below. 50 μL was harvested and added to the 150 μL/well compound plate prepared as described in the first point to dilute 1:4 the cell suspension. The cell assay plate diluted and containing 200 μL/well suspension was incubated at 37° C. in a humidified incubator with 5% CO₂.

On Day 4, 150 μL/well of fresh medium was added into a new 0.3 μL/well copy compound plates. From each well of the 200 μL/well cell assay plate, 100 μL was sampled for the cell viability measurement as described below.

Cell viability measurement. Plates containing the samples to be tested were equilibrated at room temperature for approximately 30 min and then 30 μL/well of the Promega CellTiterGlo® reagent (Promega catalog no. G7572) were added. Contents were mixed for 5 min on an orbital shaker to induce cell lysis and then incubated at room temperature for an additional 10 min to stabilize the luminescent signal (in the dark). Luminescence was read by using VictorV (Perkin Elmer) multilabel plate reader using the luminescence for 96 well plate standard protocol.

Data handling. Data was expressed as % of inhibition compared to the 0.2% DMSO negative control, and is calculated as follows: % inhibition=100−[(RLU sample)×100/(RLU average controls)]. CRCs were analysed by GraphPad (Prism) and IC₅₀ values were be calculated by non-linear regression using a 4 parameter-logistic equation. IC₅₀ (μM) values are reported in the table below.

### Example B1b

### Antileukemic Activity

The present example evaluates the compounds described herein, inhibitors of menin, for their ability to inhibit cell proliferation. The proliferation inhibitory effect was investigated in MLL fusion harboring human leukemia cell lines listed in the table below.

| Cell | MLL gene | | Cat. |
|---|---|---|---|
| Type | fusion | Supplier/Vendor | Number |
| MV4-11 | MLL-AF4 | ATCC/LCC | CRL-9591 |
| MOLM-13 | MLL-AF9 | DSMZ | ACC-554 |
| K-562 | BCR-ABL | ATCC | CCL-243 |

ATP is present in all metabolically active cells and is considered a marker for cell viability and proliferation. The metabolic cell activity was determined using the CellTiter-Glo 2.0 kit from Promega (Catalog #G9243), an ATP monitoring system based on the production of luminescence by the reaction with added UltraGlo recombinant luciferase. The amount of ATP produced is directly proportional to the number of cells present in the culture (Kawano et al., 2016, PLOS One, 8: 11 (7): e0158888). The cell proliferation assay was based on a 96 well plate format.

Test compounds were dissolved at 10 mM in DMSO with purity ≥99.9% (Sigma catalog no. D8418) and stored at −20° C. Cell lines were grown in suspension in RPMI-1640 medium (Gibco catalog no. 11875-093) supplemented with 10% of Heat Inactivated FBS (Gibco catalog no. 10082-147) and 1% Pen-Strep (Cytiva catalog no. SV30010) and cultured at 37° C. in a humidified incubator with 5% CO₂. Following thaw, cells were maintained for at least 3 passages before use in the proliferation assay.

On Day 0, the day of the experiment, cells were counted and resuspended in fresh medium at a cell density of 5×10∧4 cells/mL. Cells were seeded at 1×10∧4 cells per well in 200 μL, in 96-well cell-culture plates using MINI 96 (Integra Biosciences). Eight concentrations of test compound, in 3-fold serial dilutions, were assessed in duplicate in an individual test occasion. Appropriate volumes of the stock solutions of test compound, and/or DMSO were added to the wells using a Tecan D300e digital dispenser (Tecan Group Ltd.) to achieve the final desired concentrations. The final DMSO concentration of 0.2% was maintained across all wells of the assay plate. Wells with untreated cells (0.2% DMSO) represent 100% proliferation. Assay plates were incubated at 37° C. in a humidified incubator with 5% CO₂.

On Day 4, viable cell counts were determined by quantifying ATP content using the CellTiter-Glo kit from Promega. For this, assay plates were equilibrated to room temperature for approximately 15 min and 40 μL of the CellTiterGlo reagent added per well and contents mixed for 30 min on an orbital shaker to induce cell lysis. Luminescence was read by using Synergy Neo 2 (BioTek Instruments, Inc.) multilabel plate reader.

Results were expressed as percent of inhibition compared to the 0.2% DMSO negative control, and was calculated as follows:

$$\% inhibition = 100 - [ ( RLU of sample ) \times 100 / ( Average RLU of no - drug control ) ]$$

Dose response curves were plotted using GraphPad and IC₅₀ values were calculated by non-linear regression using 4 parameter-logistic equation. Additional data is included in Table 1.

IC₅₀ values measured on day T4 (4 days) are reported in the Table 1 below: *****≤1 nM, **** 1-10 nM, *** 10-100 nM, ** 100-500 nM, *>500 nM.

TABLE 1
IC₅₀ values of representative compounds of the invention

| | | | IC₅₀ MOLM13 | IC₅₀ MV-4: 11 | IC₅₀ K-562 |
|---|---|---|---|---|---|
| Compd ID | Structure | MW | Day 4 MEAN | Day 4 MEAN | Day 4 MEAN |
| | TABLE 1D | | | | |
| 310 | 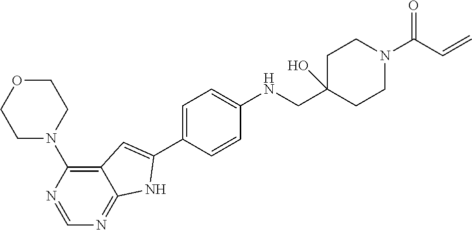 [SMILES: C=CC(=O)N1CCC(O)(CNc2ccc(-c3cc4c(N5CCOCC5)ncnc4[nH]3)cc2)CC1] | | ** | ** | * |
| | C₂₅H₃₀N₆O₃ | | | | |
| 314 | 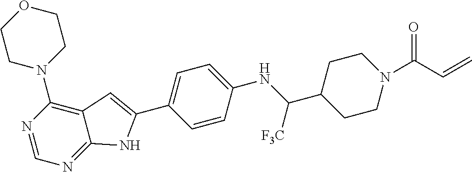 [SMILES: C=CC(=O)N1CCC(C(Nc2ccc(-c3cc4c(N5CCOCC5)ncnc4[nH]3)cc2)C(F)(F)F)CC1] | 514.55 | ** | ** | * |
| | C₂₆H₂₉F₃N₆O₂ | | | | |
| | TABLE 1E | | | | |
| 401 | 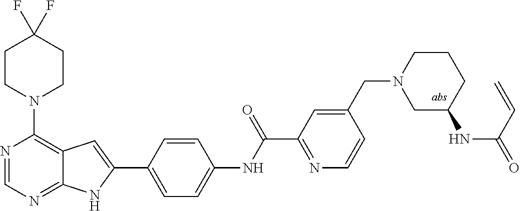 [SMILES: C=CC(=O)N[C@@H]1CCCN(Cc2ccnc(C(=O)Nc3ccc(-c4cc5c(N6CCC(F)(F)CC6)ncnc5[nH]4)cc3)c2)C1] | 600.66 | * | * | * |
| | C₃₂H₃₄F₂N₈O₂ | | | | |
| 402 | 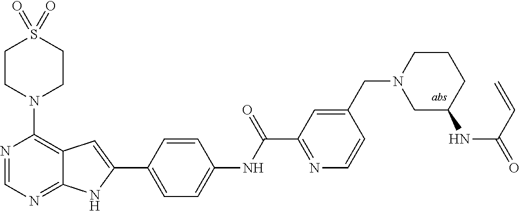 [SMILES: C=CC(=O)N[C@@H]1CCCN(Cc2ccnc(C(=O)Nc3ccc(-c4cc5c(N6CCS(=O)(=O)CC6)ncnc5[nH]4)cc3)c2)C1] | 614.72 | ** | ** | * |
| | C₃₁H₃₄N₈O₄S | | | | |
| 407 | 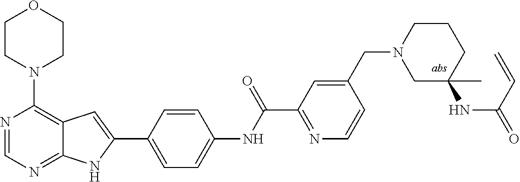 [SMILES: C=CC(=O)N[C@]1(C)CCCN(Cc2ccnc(C(=O)Nc3ccc(-c4cc5c(N6CCOCC6)ncnc5[nH]4)cc3)c2)C1] | 580.68 | ** | ** | * |
| | C₃₂H₃₆N₈O₃ | | | | |
| 419 | 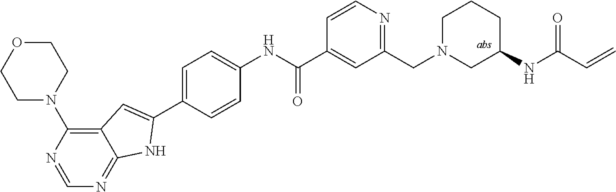 [SMILES: C=CC(=O)N[C@@H]1CCCN(Cc2cc(C(=O)Nc3ccc(-c4cc5c(N6CCOCC6)ncnc5[nH]4)cc3)ccn2)C1] | 566.67 | * | ** | * |
| | C₃₁H₃₄N₈O₃ | | | | |
| 429 | 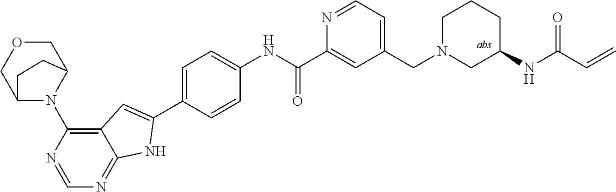 [SMILES: C=CC(=O)N[C@@H]1CCCN(Cc2ccnc(C(=O)Nc3ccc(-c4cc5c(N6C7CCC6COC7)ncnc5[nH]4)cc3)c2)C1] | 592.7 | ** | ** | * |
| | C₃₃H₃₆N₈O₃ | | | | |
| 451 | 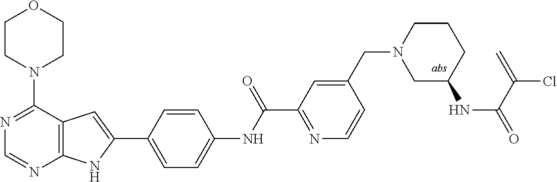 [SMILES: C=C(Cl)C(=O)N[C@@H]1CCCN(Cc2ccnc(C(=O)Nc3ccc(-c4cc5c(N6CCOCC6)ncnc5[nH]4)cc3)c2)C1] | 601.11 | *** | *** | * |
| | C₃₁H₃₃ClN₈O₃ | | | | |
| | TABLE 1G | | | | |
| 601 | 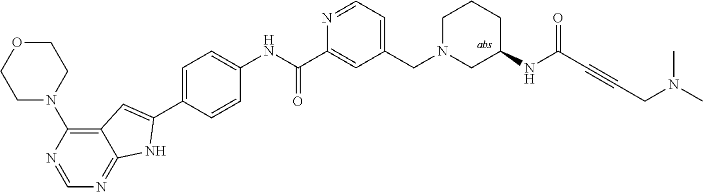 [SMILES: CN(C)CC#CC(=O)N[C@@H]1CCCN(Cc2ccnc(C(=O)Nc3ccc(-c4cc5c(N6CCOCC6)ncnc5[nH]4)cc3)c2)C1] | 621.75 | *** | *** | *** |
| | C34H39N9O3 | | | | |
| 602 | 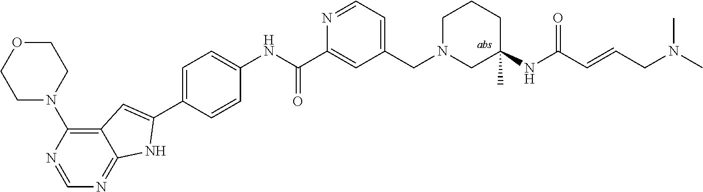 [SMILES: CN(C)C/C=C/C(=O)N[C@]1(C)CCCN(Cc2ccnc(C(=O)Nc3ccc(-c4cc5c(N6CCOCC6)ncnc5[nH]4)cc3)c2)C1] | 637.79 | ** | * | * |
| | C35H43N9O3 | | | | |
| 603 | 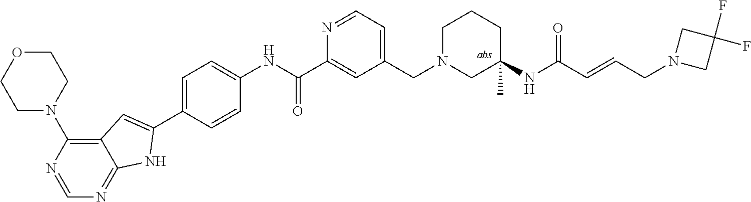 [SMILES: C[C@@]1(NC(=O)/C=C/CN2CC(F)(F)C2)CCCN(Cc2ccnc(C(=O)Nc3ccc(-c4cc5c(N6CCOCC6)ncnc5[nH]4)cc3)c2)C1] | 685.78 | ** | * | * |
| | C36H41F2N9O3 | | | | |
| 604 | 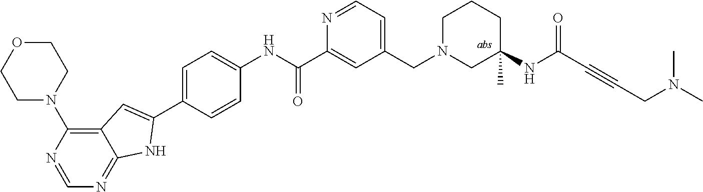 [SMILES: CN(C)CC#CC(=O)N[C@]1(C)CCCN(Cc2ccnc(C(=O)Nc3ccc(-c4cc5c(N6CCOCC6)ncnc5[nH]4)cc3)c2)C1] | 635.77 | *** | *** | *** |
| | C35H41N9O3 | | | | |
| 605 | 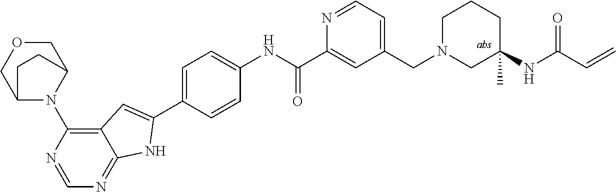 [SMILES: C=CC(=O)N[C@]1(C)CCCN(Cc2ccnc(C(=O)Nc3ccc(-c4cc5c(N6C7CCC6COC7)ncnc5[nH]4)cc3)c2)C1] | 606.73 | * | * | * |
| | C34H38N8O3 | | | | |
| 606 | 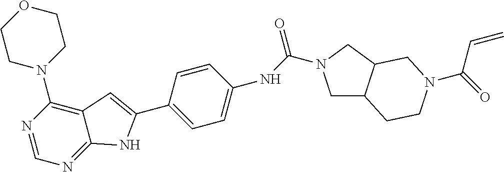 [SMILES: C=CC(=O)N1CCC2CN(C(=O)Nc3ccc(-c4cc5c(N6CCOCC6)ncnc5[nH]4)cc3)CC2C1] | 501.59 | * | * | * |
| | C27H31N7O3 | | | | |
| 607 | 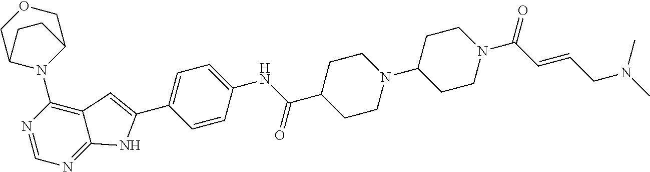 [SMILES: CN(C)C/C=C/C(=O)N1CCC(N2CCC(C(=O)Nc3ccc(-c4cc5c(N6C7CCC6COC7)ncnc5[nH]4)cc3)CC2)CC1] | 626.81 | * | * | * |
| | C35H46N8O3 | | | | |
| 608 | 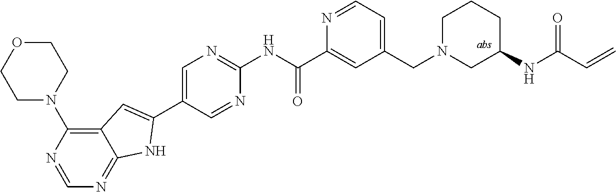 [SMILES: C=CC(=O)N[C@@H]1CCCN(Cc2ccnc(C(=O)Nc3ncc(-c4cc5c(N6CCOCC6)ncnc5[nH]4)cn3)c2)C1] | 568.64 | * | * | * |
| | C29H32N10O3 | | | | |
| 609 | 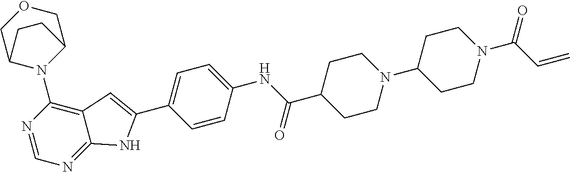 [SMILES: C=CC(=O)N1CCC(N2CCC(C(=O)Nc3ccc(-c4cc5c(N6C7CCC6COC7)ncnc5[nH]4)cc3)CC2)CC1] | 569.71 | ** | ** | * |
| | C32H39N7O3 | | | | |
| 610 | 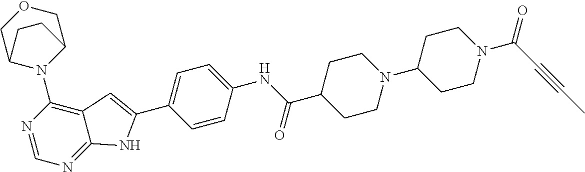 [SMILES: CC#CC(=O)N1CCC(N2CCC(C(=O)Nc3ccc(-c4cc5c(N6C7CCC6COC7)ncnc5[nH]4)cc3)CC2)CC1] | 581.72 | ** | ** | * |
| | C33H39N7O3 | | | | |
| 611 | 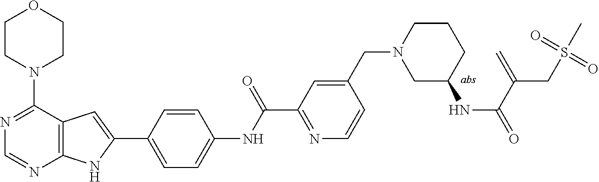 [SMILES: C=C(CS(C)(=O)=O)C(=O)N[C@@H]1CCCN(Cc2ccnc(C(=O)Nc3ccc(-c4cc5c(N6CCOCC6)ncnc5[nH]4)cc3)c2)C1] | 658.78 | *** | *** | ** |
| | C33H38N8O5S | | | | |
| 612 | 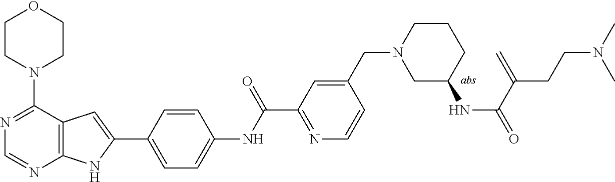 [SMILES: C=C(CCN(C)C)C(=O)N[C@@H]1CCCN(Cc2ccnc(C(=O)Nc3ccc(-c4cc5c(N6CCOCC6)ncnc5[nH]4)cc3)c2)C1] | 637.79 | ** | * | * |
| | C35H43N9O3 | | | | |
| 613 | 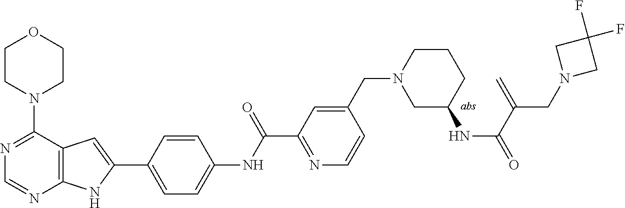 [SMILES: C=C(CN1CC(F)(F)C1)C(=O)N[C@@H]1CCCN(Cc2ccnc(C(=O)Nc3ccc(-c4cc5c(N6CCOCC6)ncnc5[nH]4)cc3)c2)C1] | 671.75 | ** | ** | * |
| | C35H39F2N9O3 | | | | |
| 614 | 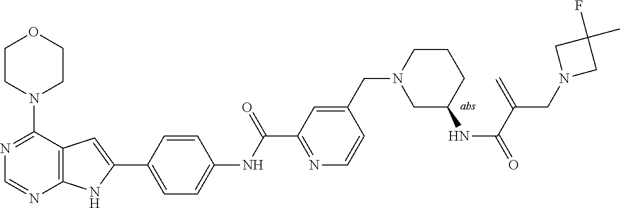 [SMILES: C=C(CN1CC(C)(F)C1)C(=O)N[C@@H]1CCCN(Cc2ccnc(C(=O)Nc3ccc(-c4cc5c(N6CCOCC6)ncnc5[nH]4)cc3)c2)C1] | 667.79 | *** | *** | ** |
| | C36H42FN9O3 | | | | |
| 615 | 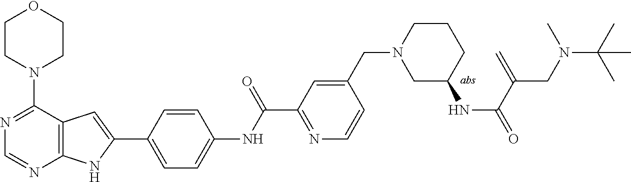 [SMILES: C=C(CN(C)C(C)(C)C)C(=O)N[C@@H]1CCCN(Cc2ccnc(C(=O)Nc3ccc(-c4cc5c(N6CCOCC6)ncnc5[nH]4)cc3)c2)C1] | 665.84 | *** | **** | *** |
| | C37H47N9O3 | | | | |
| 616 | 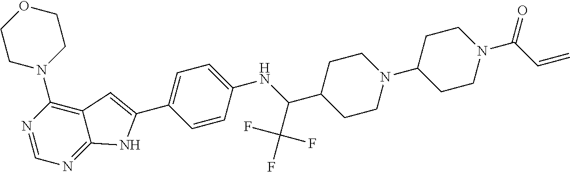 [SMILES: C=CC(=O)N1CCC(N2CCC(C(Nc3ccc(-c4cc5c(N6CCOCC6)ncnc5[nH]4)cc3)C(F)(F)F)CC2)CC1] | 597.69 | *** | *** | ** |
| | C31H38F3N7O2 | | | | |
| 617 | 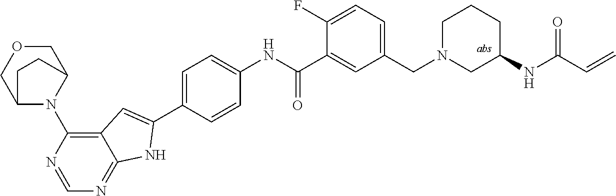 [SMILES: C=CC(=O)N[C@@H]1CCCN(Cc2ccc(F)c(C(=O)Nc3ccc(-c4cc5c(N6C7CCC6COC7)ncnc5[nH]4)cc3)c2)C1] | 609.71 | ** | ** | * |
| | C34H36FN7O3 | | | | |
| 618 | 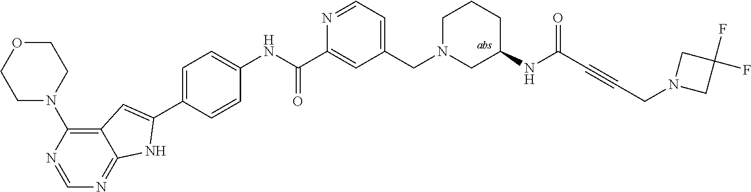 [SMILES: O=C(C#CCN1CC(F)(F)C1)N[C@@H]1CCCN(Cc2ccnc(C(=O)Nc3ccc(-c4cc5c(N6CCOCC6)ncnc5[nH]4)cc3)c2)C1] | 669.74 | *** | *** | ** |
| | C35H37F2N9O3 | | | | |
| 619 | 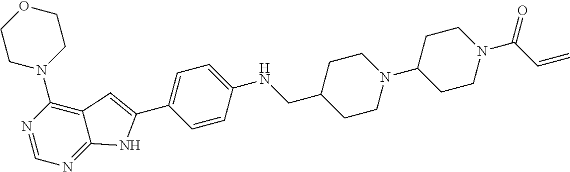 [SMILES: C=CC(=O)N1CCC(N2CCC(CNc3ccc(-c4cc5c(N6CCOCC6)ncnc5[nH]4)cc3)CC2)CC1] | 529.69 | *** | *** | ** |
| | C30H39N7O2 | | | | |
| 620 | 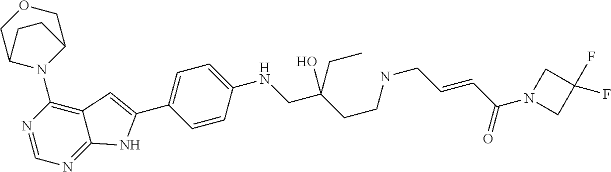 [SMILES: CCC(O)(CCNC/C=C/C(=O)N1CC(F)(F)C1)CNc1ccc(-c2cc3c(N4C5CCC4COC5)ncnc3[nH]2)cc1] | 593.68 | ** | *** | * |
| | C31H37F2N7O3 | | | | |
| 621 | 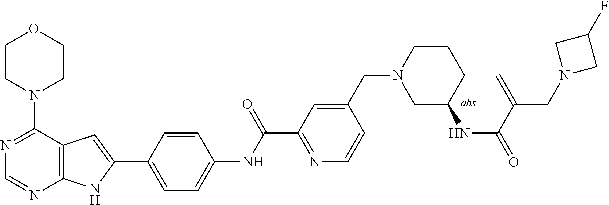 [SMILES: C=C(CN1CC(F)C1)C(=O)N[C@@H]1CCCN(Cc2ccnc(C(=O)Nc3ccc(-c4cc5c(N6CCOCC6)ncnc5[nH]4)cc3)c2)C1] | 653.76 | *** | *** | *** |
| | C35H40FN9O3 | | | | |
| 622 | 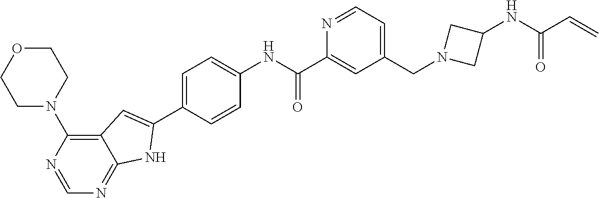 [SMILES: C=CC(=O)NC1CN(Cc2ccnc(C(=O)Nc3ccc(-c4cc5c(N6CCOCC6)ncnc5[nH]4)cc3)c2)C1] | 538.61 | ** | ** | * |
| | C29H30N8O3 | | | | |
| 623 | 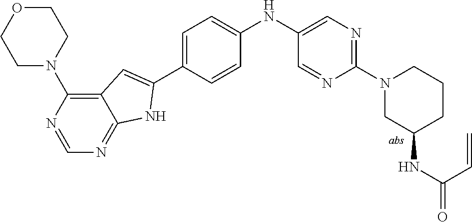 [SMILES: C=CC(=O)N[C@@H]1CCCN(c2ncc(Nc3ccc(-c4cc5c(N6CCOCC6)ncnc5[nH]4)cc3)cn2)C1] | 525.62 | *** | *** | * |
| | C28H31N9O2 | | | | |
| 624 | 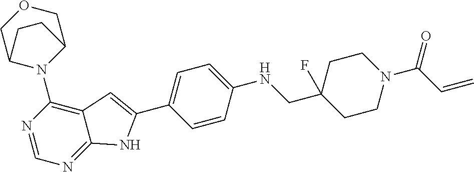 [SMILES: C=CC(=O)N1CCC(F)(CNc2ccc(-c3cc4c(N5C6CCC5COC6)ncnc4[nH]3)cc2)CC1] | 490.58 | * | * | * |
| | C27H31FN6O2 | | | | |
| 625 | 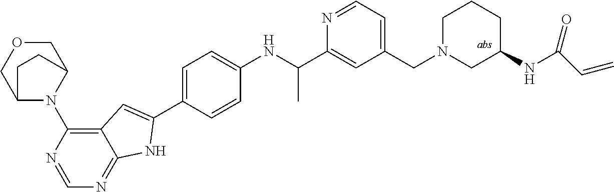 [SMILES: C=CC(=O)N[C@@H]1CCCN(Cc2ccnc(C(C)Nc3ccc(-c4cc5c(N6C7CCC6COC7)ncnc5[nH]4)cc3)c2)C1] | 592.75 | * | * | * |
| | C34H40N8O2 | | | | |
| 626 | 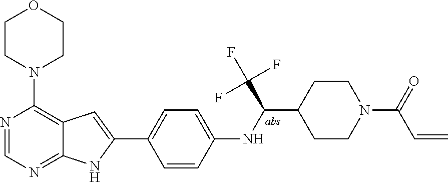 [SMILES: C=CC(=O)N1CCC([C@@H](Nc2ccc(-c3cc4c(N5CCOCC5)ncnc4[nH]3)cc2)C(F)(F)F)CC1] | 514.55 | ** | ** | * |
| | C26H29F3N6O2 | | | | |
| 627 | 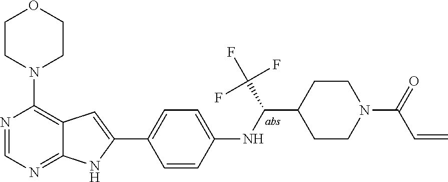 [SMILES: C=CC(=O)N1CCC([C@H](Nc2ccc(-c3cc4c(N5CCOCC5)ncnc4[nH]3)cc2)C(F)(F)F)CC1] | 514.55 | ** | ** | * |
| | C26H29F3N6O2 | | | | |
| 628 | 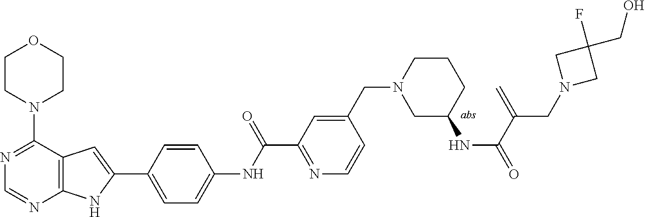 [SMILES: C=C(CN1CC(F)(CO)C1)C(=O)N[C@@H]1CCCN(Cc2ccnc(C(=O)Nc3ccc(-c4cc5c(N6CCOCC6)ncnc5[nH]4)cc3)c2)C1] | 683.79 | *** | *** | *** |
| | C36H42FN9O4 | | | | |
| 629 | 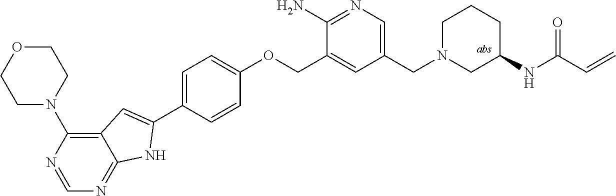 [SMILES: C=CC(=O)N[C@@H]1CCCN(Cc2cnc(N)c(COc3ccc(-c4cc5c(N6CCOCC6)ncnc5[nH]4)cc3)c2)C1] | 568.68 | ** | ** | * |
| | C31H36N8O3 | | | | |
| 630 | 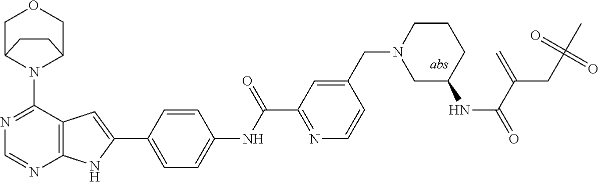 | 684.82 | *** | *** | ** |
| | C35H40N8O5S | | | | |
| 631 | 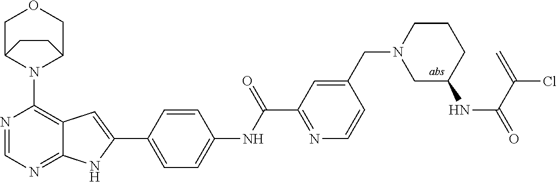 [SMILES: C=C(Cl)C(=O)N[C@@H]1CCCN(Cc2ccnc(C(=O)Nc3ccc(-c4cc5c(N6C7CCC6COC7)ncnc5[nH]4)cc3)c2)C1] | 627.15 | *** | *** | ** |
| | C33H35ClN8O3 | | | | |
| 632 | 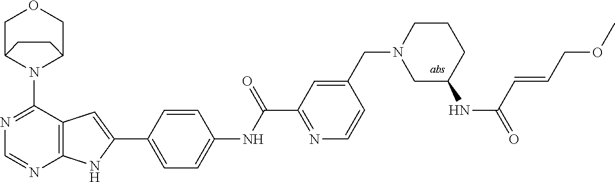 [SMILES: COC/C=C/C(=O)N[C@@H]1CCCN(Cc2ccnc(C(=O)Nc3ccc(-c4cc5c(N6C7CCC6COC7)ncnc5[nH]4)cc3)c2)C1] | 636.76 | * | * | * |
| | C35H40N8O4 | | | | |
| 633 | 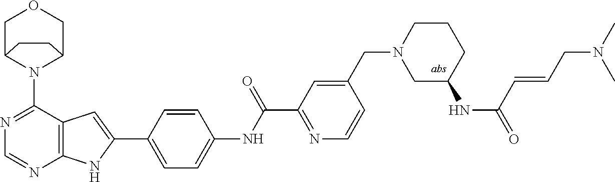 [SMILES: CN(C)C/C=C/C(=O)N[C@@H]1CCCN(Cc2ccnc(C(=O)Nc3ccc(-c4cc5c(N6C7CCC6COC7)ncnc5[nH]4)cc3)c2)C1] | 649.8 | ** | ** | * |
| | C36H43N9O3 | | | | |
| 634 | 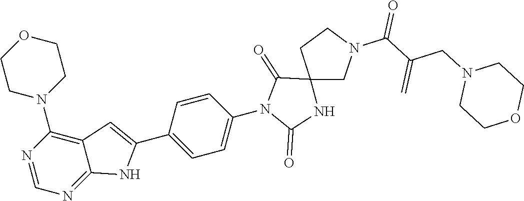 [SMILES: C=C(CN1CCOCC1)C(=O)N1CCC2(C1)NC(=O)N(c1ccc(-c3cc4c(N5CCOCC5)ncnc4[nH]3)cc1)C2=O] | 586.65 | * | * | * |
| | C30H34N8O5 | | | | |
| 635 | 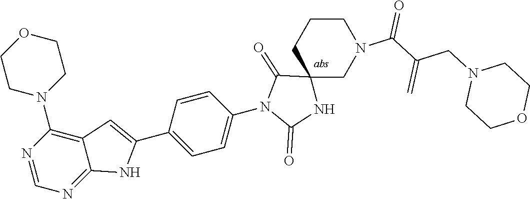 [SMILES: C=C(CN1CCOCC1)C(=O)N1CCC[C@@]2(C1)NC(=O)N(c1ccc(-c3cc4c(N5CCOCC5)ncnc4[nH]3)cc1)C2=O] | 600.68 | | | |
| | C31H36N8O5 | | | | |
| 636 | 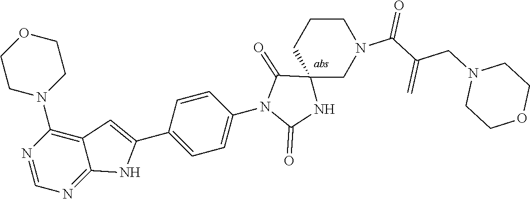 [SMILES: C=C(CN1CCOCC1)C(=O)N1CCC[C@]2(C1)NC(=O)N(c1ccc(-c3cc4c(N5CCOCC5)ncnc4[nH]3)cc1)C2=O] | 600.68 | ** | ** | * |
| | C31H36N8O5 | | | | |
| 637 | 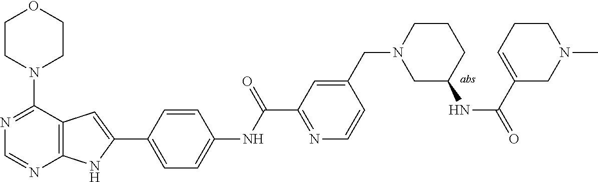 [SMILES: CN1CCC=C(C(=O)N[C@@H]2CCCN(Cc3ccnc(C(=O)Nc4ccc(-c5cc6c(N7CCOCC7)ncnc6[nH]5)cc4)c3)C2)C1] | 635.77 | ** | ** | * |
| | C35H41N9O3 | | | | |
| 638 | 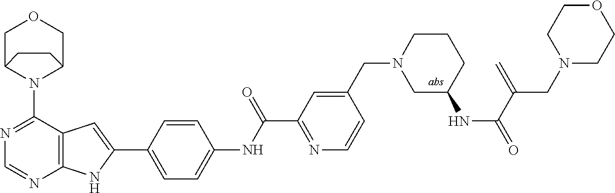 [SMILES: C=C(CN1CCOCC1)C(=O)N[C@@H]1CCCN(Cc2ccnc(C(=O)Nc3ccc(-c4cc5c(N6C7CCC6COC7)ncnc5[nH]4)cc3)c2)C1] | 691.84 | *** | *** | *** |
| | C38H45N9O4 | | | | |
| 639 | 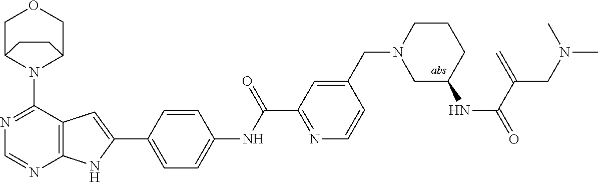 [SMILES: C=C(CN(C)C)C(=O)N[C@@H]1CCCN(Cc2ccnc(C(=O)Nc3ccc(-c4cc5c(N6C7CCC6COC7)ncnc5[nH]4)cc3)c2)C1] | 649.8 | *** | *** | *** |
| | C36H43N9O3 | | | | |
| 640 | 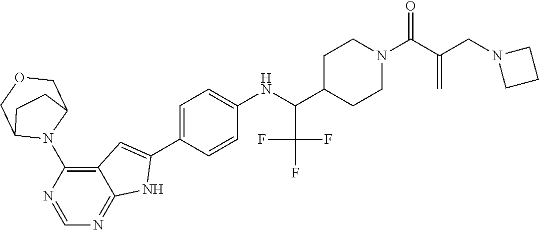 [SMILES: C=C(CN1CCC1)C(=O)N1CCC(C(Nc2ccc(-c3cc4c(N5C6CCC5COC6)ncnc4[nH]3)cc2)C(F)(F)F)CC1] | 609.7 | ** | ** | * |
| | C₃₂H₃₈F₃N₈O₂ | | | | |
| 641 | 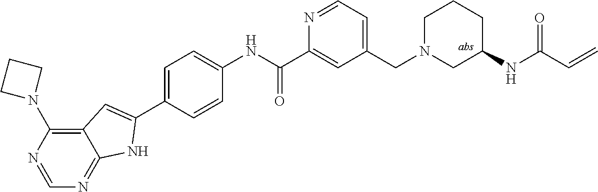 [SMILES: C=CC(=O)N[C@@H]1CCCN(Cc2ccnc(C(=O)Nc3ccc(-c4cc5c(N6CCC6)ncnc5[nH]4)cc3)c2)C1] | 536.64 | * | * | * |
| | C₃₀H₃₂N₈O₂ | | | | |
| 642 | 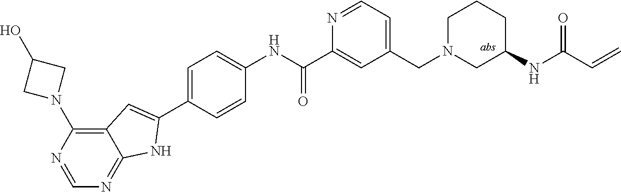 [SMILES: C=CC(=O)N[C@@H]1CCCN(Cc2ccnc(C(=O)Nc3ccc(-c4cc5c(N6CC(O)C6)ncnc5[nH]4)cc3)c2)C1] | 552.64 | * | * | * |
| | C₃₀H₃₂N₈O₃ | | | | |
| 643 | 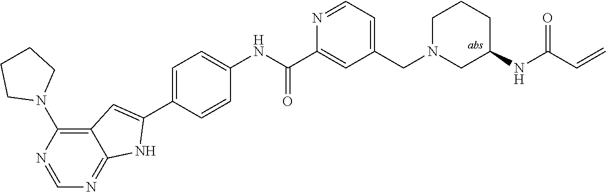 [SMILES: C=CC(=O)N[C@@H]1CCCN(Cc2ccnc(C(=O)Nc3ccc(-c4cc5c(N6CCCC6)ncnc5[nH]4)cc3)c2)C1] | 550.67 | * | * | * |
| | C₃₁H₃₄N₈O₂ | | | | |
| 644 | 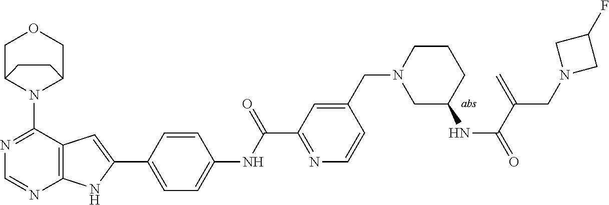 [SMILES: C=C(CN1CC(F)C1)C(=O)N[C@@H]1CCCN(Cc2ccnc(C(=O)Nc3ccc(-c4cc5c(N6C7CCC6COC7)ncnc5[nH]4)cc3)c2)C1] | 679.8 | *** | *** | *** |
| | C₃₇H₄₂FN₉O₃ | | | | |
| 645 | 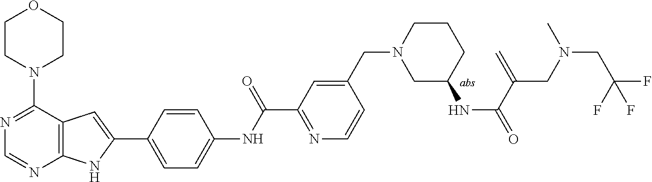 [SMILES: C=C(CN(C)CC(F)(F)F)C(=O)N[C@@H]1CCCN(Cc2ccnc(C(=O)Nc3ccc(-c4cc5c(N6CCOCC6)ncnc5[nH]4)cc3)c2)C1] | 691.76 | * | * | * |
| | C₃₅H₄₀F₃N₉O₃ | | | | |
| 646 | 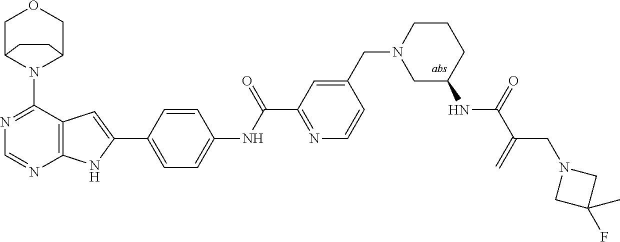 [SMILES: C=C(CN1CC(C)(F)C1)C(=O)N[C@@H]1CCCN(Cc2ccnc(C(=O)Nc3ccc(-c4cc5c(N6C7CCC6COC7)ncnc5[nH]4)cc3)c2)C1] | 693.83 | *** | *** | *** |
| | C₃₈H₄₄FN₉O₃ | | | | |
| | TABLE 1H | | | | |
| 701 | 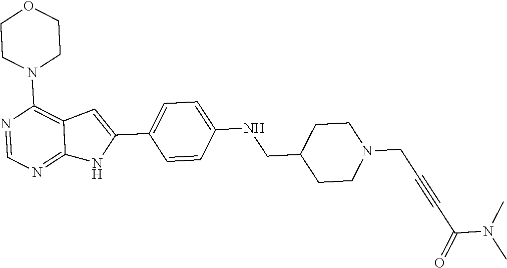 [SMILES: CN(C)C(=O)C#CCN1CCC(CNc2ccc(-c3cc4c(N5CCOCC5)ncnc4[nH]3)cc2)CC1] | 501.63 | *** | *** | ** |
| | C28H35N7O2 | | | | |
| 702 | 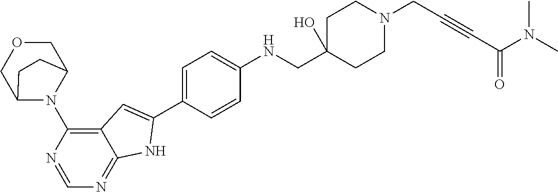 [SMILES: CN(C)C(=O)C#CCN1CCC(O)(CNc2ccc(-c3cc4c(N5C6CCC5COC6)ncnc4[nH]3)cc2)CC1] | 543.67 | ** | *** | * |
| | C30H37N7O3 | | | | |
| 703 | 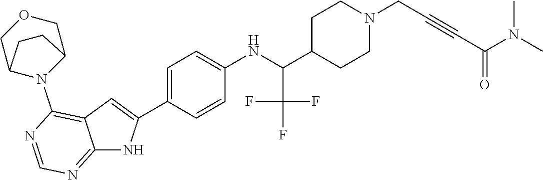 [SMILES: CN(C)C(=O)C#CCN1CCC(C(Nc2ccc(-c3cc4c(N5C6CCC5COC6)ncnc4[nH]3)cc2)C(F)(F)F)CC1] | 595.67 | ** | ** | ** |
| | C₃₁H₃₆F₃N₇O₂ | | | | |
| 704 | 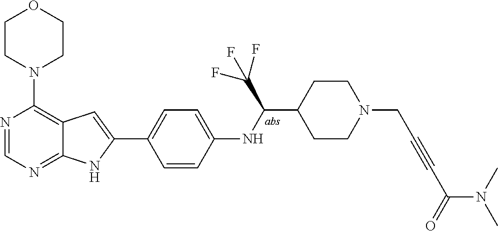 [SMILES: CN(C)C(=O)C#CCN1CCC([C@@H](Nc2ccc(-c3cc4c(N5CCOCC5)ncnc4[nH]3)cc2)C(F)(F)F)CC1] | 569.63 | *** | *** | * |
| | C29H34F3N7O2 | | | | |
| 705 | 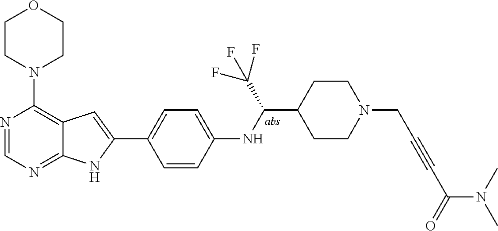 [SMILES: CN(C)C(=O)C#CCN1CCC([C@H](Nc2ccc(-c3cc4c(N5CCOCC5)ncnc4[nH]3)cc2)C(F)(F)F)CC1] | 569.63 | *** | *** | ** |
| | C29H34F3N7O2 | | | | |
| 706 | 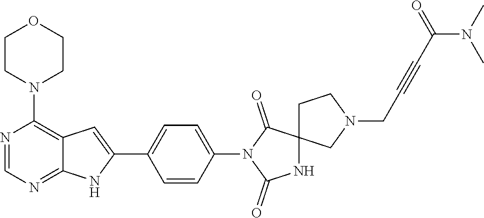 [SMILES: CN(C)C(=O)C#CCN1CCC2(C1)NC(=O)N(c1ccc(-c3cc4c(N5CCOCC5)ncnc4[nH]3)cc1)C2=O] | 542.6 | ** | ** | * |
| | C28H30N8O4 | | | | |
| | TABLE 1J | | | | |
| 801 | 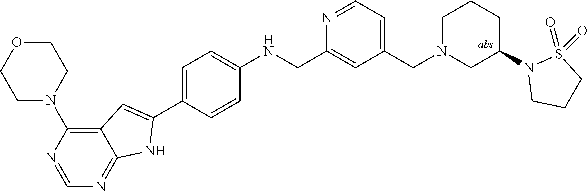 [SMILES: O=S1(=O)CCCN1[C@@H]1CCCN(Cc2ccnc(CNc3ccc(-c4cc5c(N6CCOCC6)ncnc5[nH]4)cc3)c2)C1] | 602.76 | * | * | * |
| | C31H38N8O3S | | | | |
| 802 | 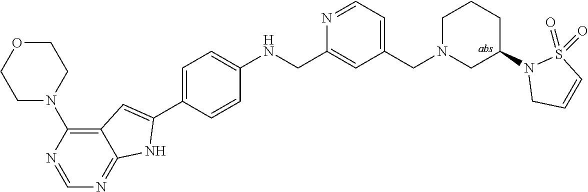 [SMILES: O=S1(=O)C=CCN1[C@@H]1CCCN(Cc2ccnc(CNc3ccc(-c4cc5c(N6CCOCC6)ncnc5[nH]4)cc3)c2)C1] | 600.74 | ** | ** | * |
| | C31H36N8O3S | | | | |
| 803 | 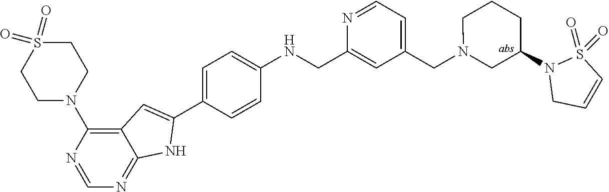 [SMILES: O=S1(=O)CCN(c2ncnc3[nH]c(-c4ccc(NCc5cc(CN6CCC[C@@H](N7CC=CS7(=O)=O)C6)ccn5)cc4)cc23)CC1] | 648.8 | ** | ** | * |
| | C31H36N8O4S2 | | | | |
| | TABLE 1K | | | | |
| 901 | 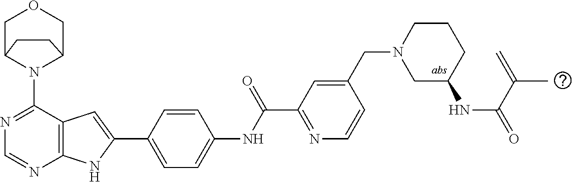 [SMILES: C=C(C)C(=O)N[C@@H]1CCCN(Cc2ccnc(C(=O)Nc3ccc(-c4cc5c(N6C7CCC6COC7)ncnc5[nH]4)cc3)c2)C1] | 610.69 | ** | ** | * |
| | C33H35FN8O3 | | | | |
| 902 | 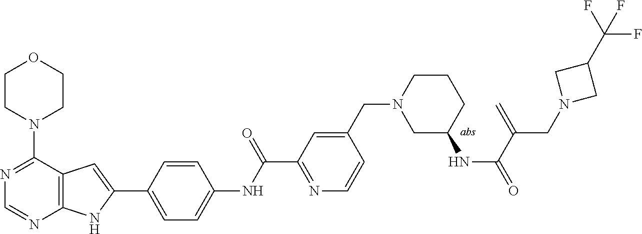 [SMILES: C=C(CN1CC(C(F)(F)F)C1)C(=O)N[C@@H]1CCCN(Cc2ccnc(C(=O)Nc3ccc(-c4cc5c(N6CCOCC6)ncnc5[nH]4)cc3)c2)C1] | 703.77 | ** | ** | ** |
| | C36H40F3N9O3 | | | | |
| 903 | 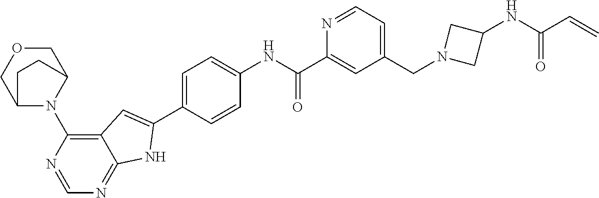 [SMILES: C=CC(=O)NC1CN(Cc2ccnc(C(=O)Nc3ccc(-c4cc5c(N6C7CCC6COC7)ncnc5[nH]4)cc3)c2)C1] | 564.65 | ** | ** | * |
| | C31H32N8O3 | | | | |
| 904 | 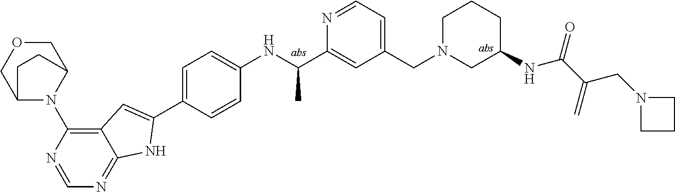 [SMILES: C=C(CN1CCC1)C(=O)N[C@@H]1CCCN(Cc2ccnc([C@@H](C)Nc3ccc(-c4cc5c(N6C7CCC6COC7)ncnc5[nH]4)cc3)c2)C1] | 661.85 | ** | ** | * |
| | C38H47N9O2 | | | | |
| 905 | 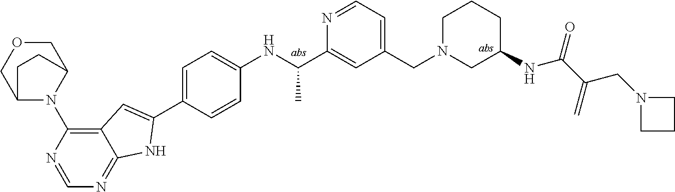 [SMILES: C=C(CN1CCC1)C(=O)N[C@@H]1CCCN(Cc2ccnc([C@H](C)Nc3ccc(-c4cc5c(N6C7CCC6COC7)ncnc5[nH]4)cc3)c2)C1] | 661.85 | ** | ** | * |
| | C38H47N9O2 | | | | |
| 906 | 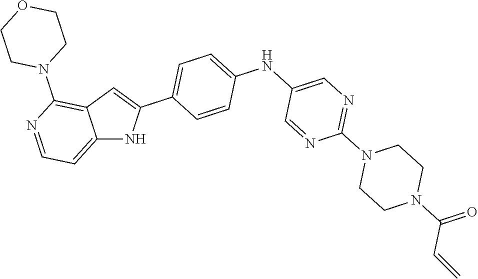 [SMILES: C=CC(=O)N1CCN(c2ncc(Nc3ccc(-c4cc5c(N6CCOCC6)nccc5[nH]4)cc3)cn2)CC1] | 511.59 | *** | *** | * |
| | C27H29N9O2 | | | | |
| 907 | 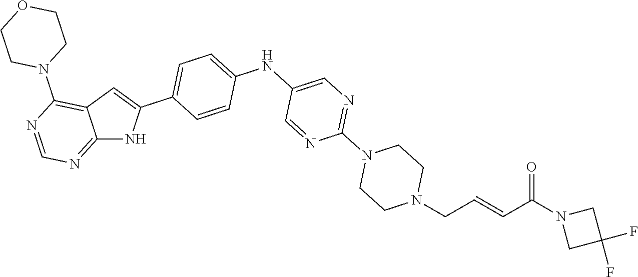 [SMILES: O=C(/C=C/CN1CCN(c2ncc(Nc3ccc(-c4cc5c(N6CCOCC6)ncnc5[nH]4)cc3)cn2)CC1)N1CC(F)(F)C1] | 616.68 | ** | *** | * |
| | C31H34F2N10O2 | | | | |
| 908 | 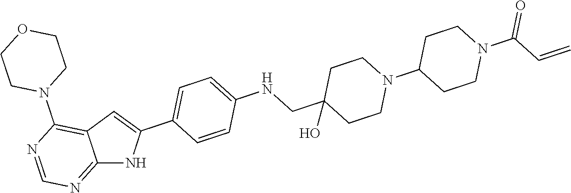 [SMILES: C=CC(=O)N1CCC(N2CCC(O)(CNc3ccc(-c4cc5c(N6CCOCC6)ncnc5[nH]4)cc3)CC2)CC1] | 545.69 | ** | *** | * |
| | C30H39N7O3 | | | | |
| 909 | 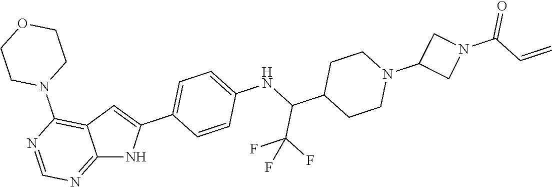 [SMILES: C=CC(=O)N1CC(N2CCC(C(Nc3ccc(-c4cc5c(N6CCOCC6)ncnc5[nH]4)cc3)C(F)(F)F)CC2)C1] | 569.63 | *** | *** | * |
| | C29H34F3N7O2 | | | | |
| 910 | 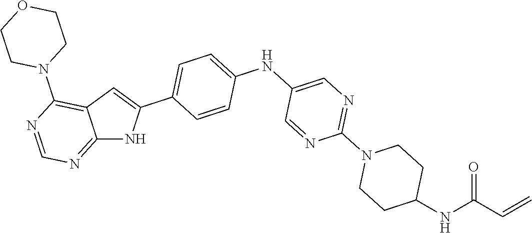 [SMILES: C=CC(=O)NC1CCN(c2ncc(Nc3ccc(-c4cc5c(N6CCOCC6)ncnc5[nH]4)cc3)cn2)CC1] | 525.62 | ** | ** | * |
| | C28H31N9O2 | | | | |
| 911 | 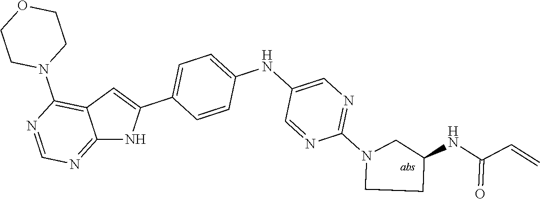 [SMILES: C=CC(=O)N[C@H]1CCN(c2ncc(Nc3ccc(-c4cc5c(N6CCOCC6)ncnc5[nH]4)cc3)cn2)C1] | 511.59 | ** | ** | * |
| | C27H29N9O2 | | | | |
| 912 | 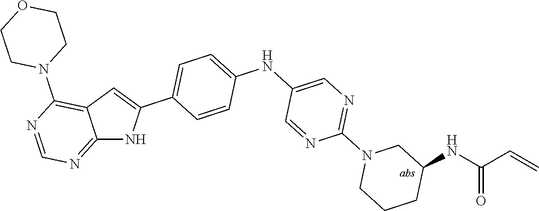 [SMILES: C=CC(=O)N[C@H]1CCCN(c2ncc(Nc3ccc(-c4cc5c(N6CCOCC6)ncnc5[nH]4)cc3)cn2)C1] | 525.62 | ** | ** | * |
| | C28H31N9O2 | | | | |
| 913 | 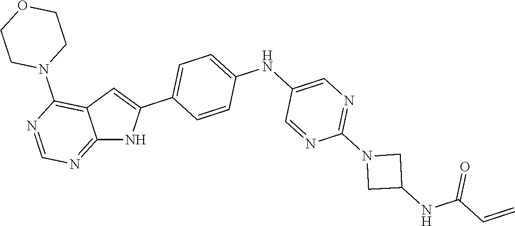 [SMILES: C=CC(=O)NC1CN(c2ncc(Nc3ccc(-c4cc5c(N6CCOCC6)ncnc5[nH]4)cc3)cn2)C1] | 497.56 | ** | ** | * |
| | C26H27N9O2 | | | | |
| 914 | 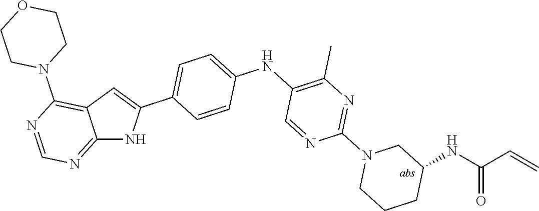 [SMILES: C=CC(=O)N[C@@H]1CCCN(c2ncc(Nc3ccc(-c4cc5c(N6CCOCC6)ncnc5[nH]4)cc3)c(C)n2)C1] | 539.64 | *** | *** | * |
| | C29H33N9O2 | | | | |
| 915 | 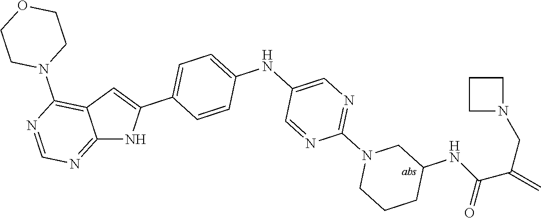 [SMILES: C=C(CN1CCC1)C(=O)NC1CCCN(c2ncc(Nc3ccc(-c4cc5c(N6CCOCC6)ncnc5[nH]4)cc3)cn2)C1] | 594.72 | *** | *** | ** |
| | C32H38N10O2 | | | | |
| 916 | 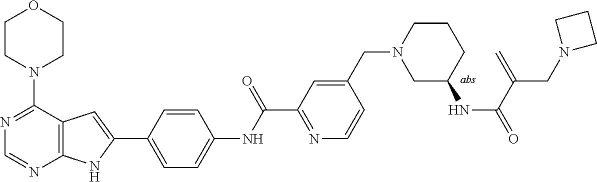 [SMILES: C=C(CN1CCC1)C(=O)N[C@@H]1CCCN(Cc2ccnc(C(=O)Nc3ccc(-c4cc5c(N6CCOCC6)ncnc5[nH]4)cc3)c2)C1] | 635.77 | *** | *** | ** |
| | C35H41N9O3 | | | | |
| 917 | 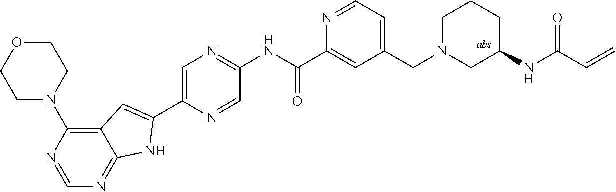 [SMILES: C=CC(=O)N[C@@H]1CCCN(Cc2ccnc(C(=O)Nc3cnc(-c4cc5c(N6CCOCC6)ncnc5[nH]4)cn3)c2)C1] | 568.64 | * | * | * |
| | C29H32N10O3 | | | | |
| 918 | 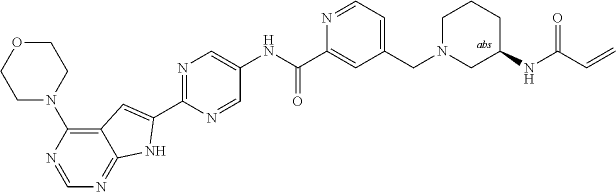 [SMILES: C=CC(=O)N[C@@H]1CCCN(Cc2ccnc(C(=O)Nc3cnc(-c4cc5c(N6CCOCC6)ncnc5[nH]4)nc3)c2)C1] | 568.64 | * | * | * |
| | C29H32N10O3 | | | | |
| 919 | 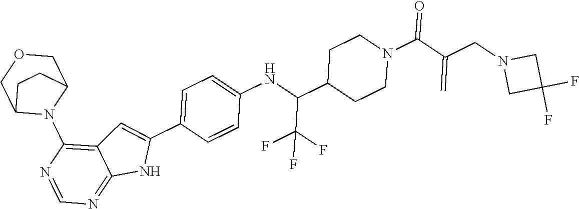 [SMILES: C=C(CN1CC(F)(F)C1)C(=O)N1CCC(C(Nc2ccc(-c3cc4c(N5C6CCC5COC6)ncnc4[nH]3)cc2)C(F)(F)F)CC1] | 645.68 | * | * | * |
| | C32H36F5N7O2 | | | | |
| 920 | 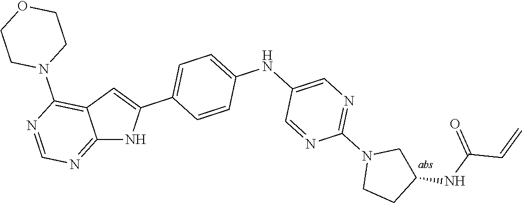 [SMILES: C=CC(=O)N[C@@H]1CCN(c2ncc(Nc3ccc(-c4cc5c(N6CCOCC6)ncnc5[nH]4)cc3)cn2)C1] | 511.59 | ** | ** | * |
| | C27H29N9O2 | | | | |
| 921 | 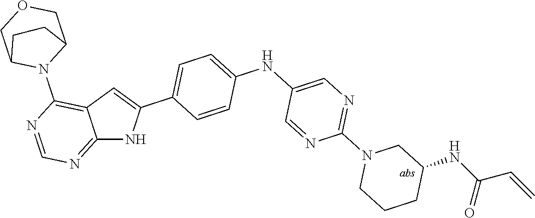 [SMILES: C=CC(=O)N[C@@H]1CCCN(c2ncc(Nc3ccc(-c4cc5c(N6C7CCC6COC7)ncnc5[nH]4)cc3)cn2)C1] | 551.65 | ** | ** | * |
| | C30H33N9O2 | | | | |
| 922 | 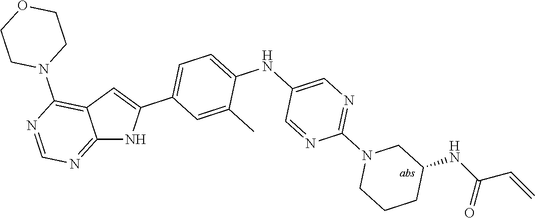 [SMILES: C=CC(=O)N[C@@H]1CCCN(c2ncc(Nc3ccc(-c4cc5c(N6CCOCC6)ncnc5[nH]4)cc3C)cn2)C1] | 539.64 | ** | ** | * |
| | C29H33N9O2 | | | | |
| 923 | 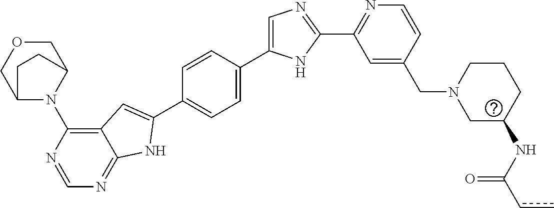 | 615.74 | * | * | * |
| | C35H37N9O2 | | | | |
| 924 | 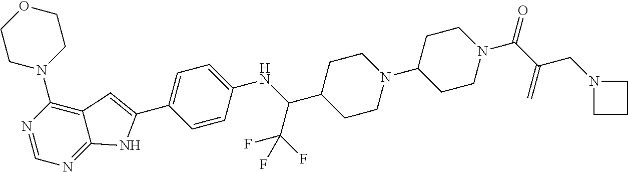 [SMILES: C=C(CN1CCC1)C(=O)N1CCC(N2CCC(C(Nc3ccc(-c4cc5c(N6CCOCC6)ncnc5[nH]4)cc3)C(F)(F)F)CC2)CC1] | 666.79 | ** | *** | * |
| | C35H45F3N8O2 | | | | |
| 925 | 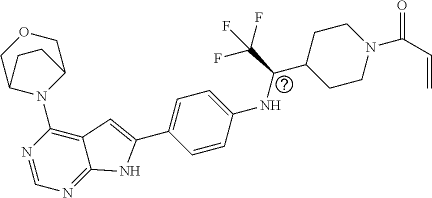 [SMILES: C=CC(=O)N1CCC([C@@H](Nc2ccc(-c3cc4c(N5C6CCC5COC6)ncnc4[nH]3)cc2)C(F)(F)F)CC1] | 540.59 | * | * | * |
| | C28H31F3N6O2 | | | | |
| 926 | 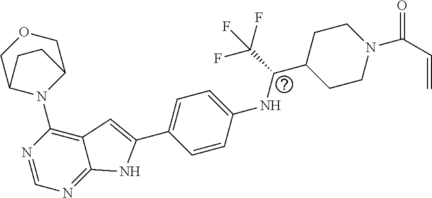 [SMILES: C=CC(=O)N1CCC([C@H](Nc2ccc(-c3cc4c(N5C6CCC5COC6)ncnc4[nH]3)cc2)C(F)(F)F)CC1] | 540.59 | * | * | * |
| | C28H31F3N6O2 | | | | |
| 927 | 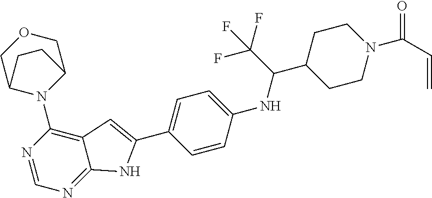 [SMILES: C=CC(=O)N1CCC(C(Nc2ccc(-c3cc4c(N5C6CCC5COC6)ncnc4[nH]3)cc2)C(F)(F)F)CC1] | 540.59 | * | * | * |
| | C28H31F3N6O2 | | | | |
| 928 | 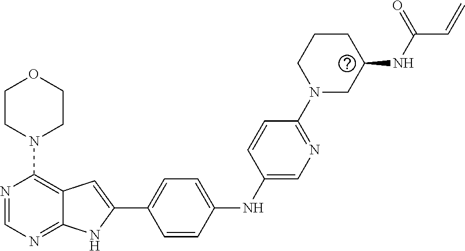 | 524.63 | ** | ** | * |
| | C29H32N8O2 | | | | |
| 929 | 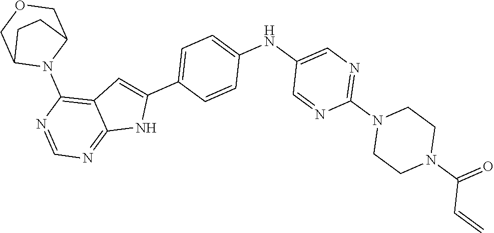 [SMILES: C=CC(=O)N1CCN(c2ncc(Nc3ccc(-c4cc5c(N6C7CCC6COC7)ncnc5[nH]4)cc3)cn2)CC1] | 537.63 | ** | *** | * |
| | C29H31N9O2 | | | | |
| 930 | 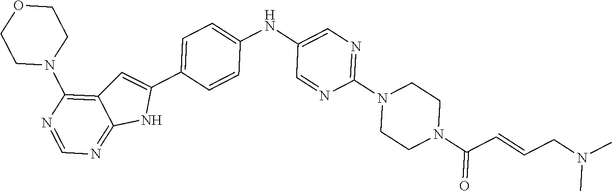 [SMILES: CN(C)C/C=C/C(=O)N1CCN(c2ncc(Nc3ccc(-c4cc5c(N6CCOCC6)ncnc5[nH]4)cc3)cn2)CC1] | 568.69 | ** | *** | * |
| | C30H36N10O2 | | | | |
| 931 | 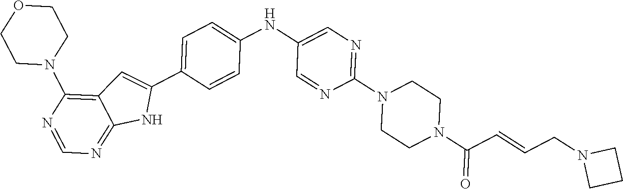 [SMILES: O=C(/C=C/CN1CCC1)N1CCN(c2ncc(Nc3ccc(-c4cc5c(N6CCOCC6)ncnc5[nH]4)cc3)cn2)CC1] | 580.7 | *** | *** | * |
| | C31H36N10O2 | | | | |
| 932 | 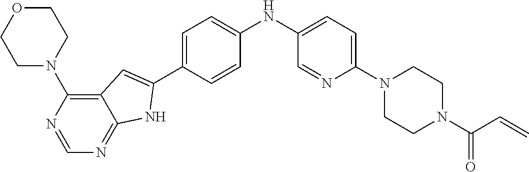 [SMILES: C=CC(=O)N1CCN(c2ccc(Nc3ccc(-c4cc5c(N6CCOCC6)ncnc5[nH]4)cc3)cn2)CC1] | 510.6 | ** | *** | * |
| | C28H30N8O2 | | | | |
| 933 | 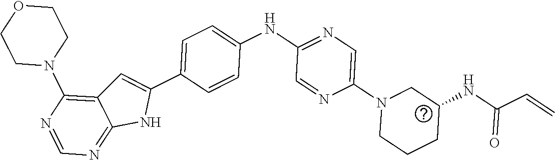 [SMILES: C=CC(=O)N[C@@H]1CCCN(c2cnc(Nc3ccc(-c4cc5c(N6CCOCC6)ncnc5[nH]4)cc3)cn2)C1] | 525.62 | | ** | * |
| | C28H31N9O2 | | | | |
| 934 | 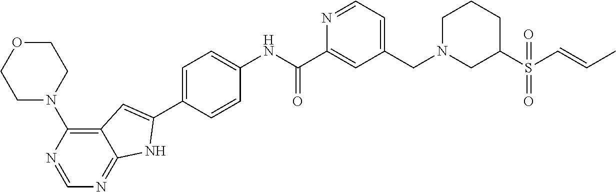 [SMILES: C/C=C/S(=O)(=O)C1CCCN(Cc2ccnc(C(=O)Nc3ccc(-c4cc5c(N6CCOCC6)ncnc5[nH]4)cc3)c2)C1] | 601.73 | | ** | * |
| | C31H35N7O4S | | | | |
| 935 | 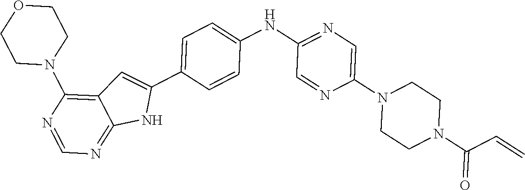 [SMILES: C=CC(=O)N1CCN(c2cnc(Nc3ccc(-c4cc5c(N6CCOCC6)ncnc5[nH]4)cc3)cn2)CC1] | 511.59 | | ** | * |
| | C27H29N9O2 | | | | |
| 936 | 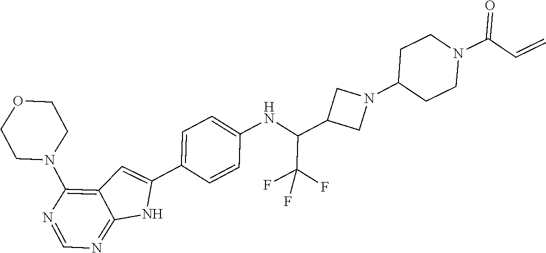 [SMILES: C=CC(=O)N1CCC(N2CC(C(Nc3ccc(-c4cc5c(N6CCOCC6)ncnc5[nH]4)cc3)C(F)(F)F)C2)CC1] | 569.63 | *** | *** | * |
| | C29H34F3N7O2 | | | | |
| 937 | 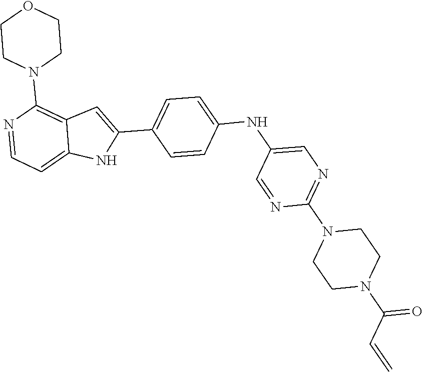 [SMILES: C=CC(=O)N1CCN(c2ncc(Nc3ccc(-c4cc5c(N6CCOCC6)nccc5[nH]4)cc3)cn2)CC1] | 510.6 | * | ** | * |
| | C28H30N8O2 | | | | |
| 938 | 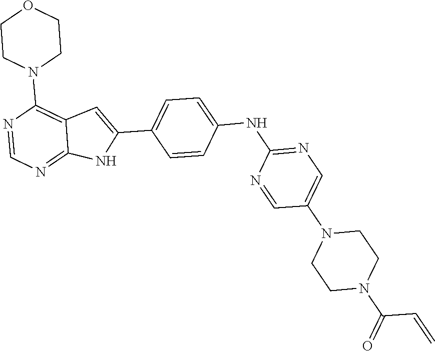 [SMILES: C=CC(=O)N1CCN(c2cnc(Nc3ccc(-c4cc5c(N6CCOCC6)ncnc5[nH]4)cc3)nc2)CC1] | 511.59 | | ** | * |
| | C27H29N9O2 | | | | |
| 939 | 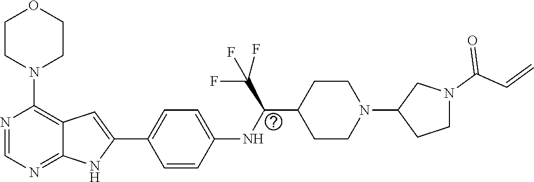 [SMILES: C=CC(=O)N1CCC(N2CCC([C@@H](Nc3ccc(-c4cc5c(N6CCOCC6)ncnc5[nH]4)cc3)C(F)(F)F)CC2)C1] | 583.66 | ** | *** | * |
| | C30H36F3N7O2 | | | | |
| 940 | 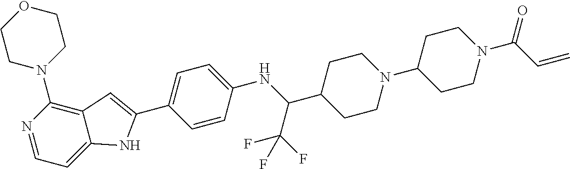 [SMILES: C=CC(=O)N1CCC(N2CCC(C(Nc3ccc(-c4cc5c(N6CCOCC6)nccc5[nH]4)cc3)C(F)(F)F)CC2)CC1] | 596.7 | * | * | * |
| | C32H39F3N6O2 | | | | |
| 941 | 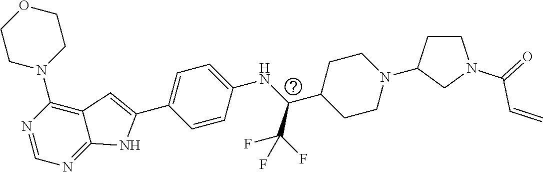 [SMILES: C=CC(=O)N1CCC(N2CCC([C@H](Nc3ccc(-c4cc5c(N6CCOCC6)ncnc5[nH]4)cc3)C(F)(F)F)CC2)C1] | 583.66 | *** | *** | * |
| | C30H36F3N7O2 | | | | |
| 942 | 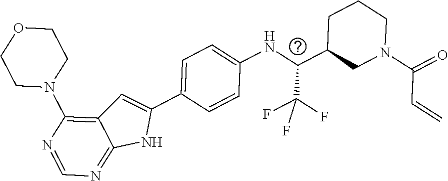 [SMILES: C=CC(=O)N1CCC[C@H]([C@@H](Nc2ccc(-c3cc4c(N5CCOCC5)ncnc4[nH]3)cc2)C(F)(F)F)C1] | 514.55 | | ** | * |
| | C26H29F3N6O2 | | | | |
| 943 | 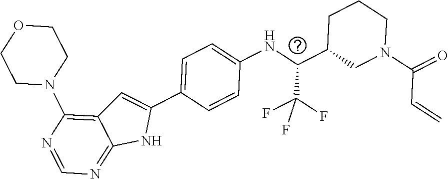 [SMILES: C=CC(=O)N1CCC[C@@H]([C@@H](Nc2ccc(-c3cc4c(N5CCOCC5)ncnc4[nH]3)cc2)C(F)(F)F)C1] | 514.55 | | * | * |
| | C26H29F3N6O2 | | | | |
| 944 | 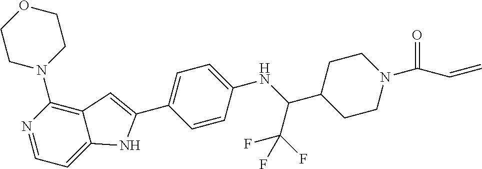 [SMILES: C=CC(=O)N1CCC(C(Nc2ccc(-c3cc4c(N5CCOCC5)nccc4[nH]3)cc2)C(F)(F)F)CC1] | 513.57 | | * | * |
| | C27H30F3N5O2 | | | | |
| 945 | 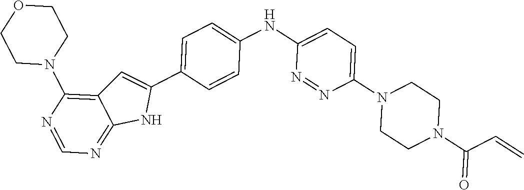 [SMILES: C=CC(=O)N1CCN(c2ccc(Nc3ccc(-c4cc5c(N6CCOCC6)ncnc5[nH]4)cc3)nn2)CC1] | 511.59 | | * | * |
| | C27H29N9O2 | | | | |
| 946 | 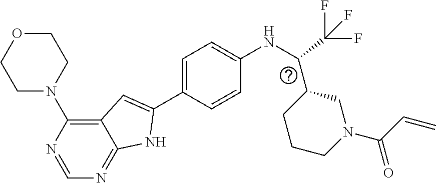 [SMILES: C=CC(=O)N1CCC[C@H]([C@H](Nc2ccc(-c3cc4c(N5CCOCC5)ncnc4[nH]3)cc2)C(F)(F)F)C1] | 514.55 | | ** | * |
| | C26H29F3N6O2 | | | | |
| 947 | 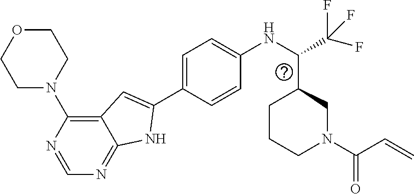 [SMILES: C=CC(=O)N1CCC[C@@H]([C@H](Nc2ccc(-c3cc4c(N5CCOCC5)ncnc4[nH]3)cc2)C(F)(F)F)C1] | 514.55 | | ** | * |
| | C26H29F3N6O2 | | | | |
| 948 | 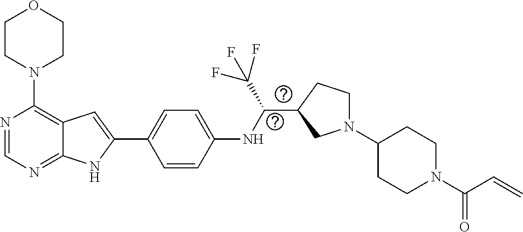 [SMILES: C=CC(=O)N1CCC(N2CC[C@H]([C@H](Nc3ccc(-c4cc5c(N6CCOCC6)ncnc5[nH]4)cc3)C(F)(F)F)C2)CC1] | 583.66 | *** | *** | * |
| | C30H36F3N7O2 | | | | |
| 949 | 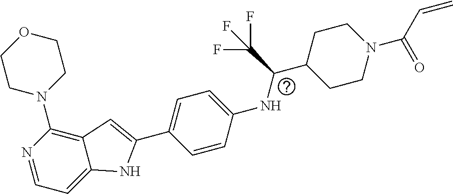 [SMILES: C=CC(=O)N1CCC([C@@H](Nc2ccc(-c3cc4c(N5CCOCC5)nccc4[nH]3)cc2)C(F)(F)F)CC1] | 513.57 | | * | * |
| | C27H30F3N5O2 | | | | |
| 950 | 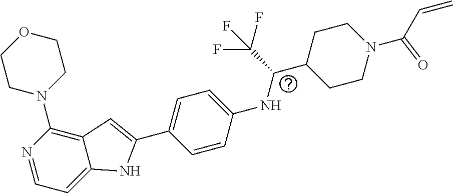 [SMILES: C=CC(=O)N1CCC([C@H](Nc2ccc(-c3cc4c(N5CCOCC5)nccc4[nH]3)cc2)C(F)(F)F)CC1] | 513.57 | | * | * |
| | C27H30F3N5O2 | | | | |
| 951 | 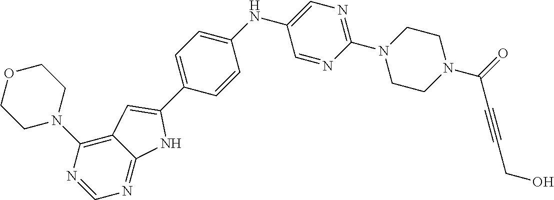 [SMILES: O=C(C#CCO)N1CCN(c2ncc(Nc3ccc(-c4cc5c(N6CCOCC6)ncnc5[nH]4)cc3)cn2)CC1] | 539.6 | *** | *** | * |
| | C28H29N9O3 | | | | |
| 952 | 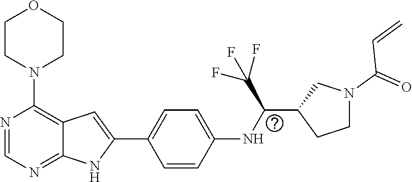 [SMILES: C=CC(=O)N1CC[C@H]([C@@H](Nc2ccc(-c3cc4c(N5CCOCC5)ncnc4[nH]3)cc2)C(F)(F)F)C1] | 500.53 | | ** | * |
| | C25H27F3N6O2 | | | | |
| 953 | 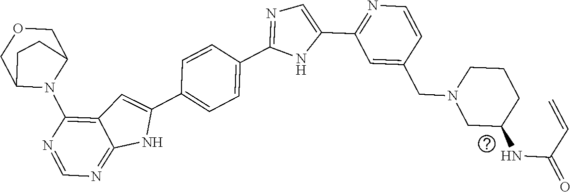 [SMILES: C=CC(=O)N[C@@H]1CCCN(Cc2ccnc(-c3cnc(-c4ccc(-c5cc6c(N7C8CCC7COC8)ncnc6[nH]5)cc4)[nH]3)c2)C1] | 615.74 | | * | * |
| | C35H37N9O2 | | | | |
| 954 | 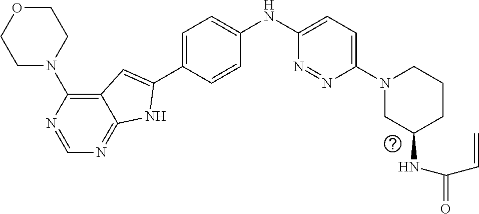 [SMILES: C=CC(=O)N[C@@H]1CCCN(c2ccc(Nc3ccc(-c4cc5c(N6CCOCC6)ncnc5[nH]4)cc3)nn2)C1] | 525.62 | | * | * |
| | C28H31N9O2 | | | | |
| 955 | 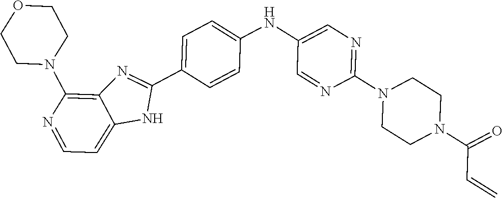 [SMILES: C=CC(=O)N1CCN(c2ncc(Nc3ccc(-c4nc5c(N6CCOCC6)nccc5[nH]4)cc3)cn2)CC1] | 511.59 | | * | * |
| | C27H29N9O2 | | | | |
| 956 | 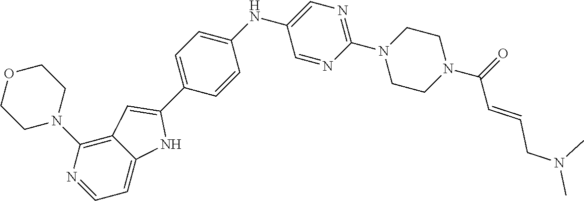 [SMILES: CN(C)C/C=C/C(=O)N1CCN(c2ncc(Nc3ccc(-c4cc5c(N6CCOCC6)nccc5[nH]4)cc3)cn2)CC1] | 567.7 | | ** | * |
| | C31H37N9O2 | | | | |
| 957 | 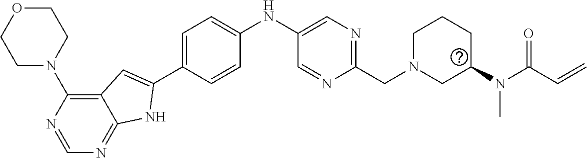 [SMILES: C=CC(=O)N(C)[C@@H]1CCCN(Cc2ncc(Nc3ccc(-c4cc5c(N6CCOCC6)ncnc5[nH]4)cc3)cn2)C1] | 553.67 | *** | *** | * |
| | C30H35N9O2 | | | | |
| 958 | 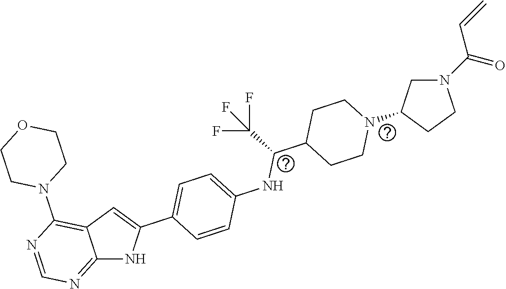 [SMILES: C=CC(=O)N1CC[C@H](N2CCC([C@H](Nc3ccc(-c4cc5c(N6CCOCC6)ncnc5[nH]4)cc3)C(F)(F)F)CC2)C1] | 583.66 | *** | *** | * |
| | C30H36F3N7O2 | | | | |
| 959 | 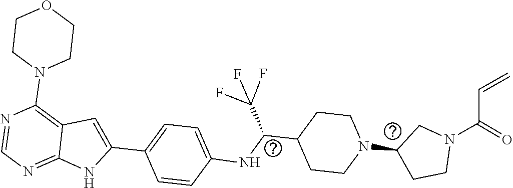 [SMILES: C=CC(=O)N1CC[C@@H](N2CCC([C@H](Nc3ccc(-c4cc5c(N6CCOCC6)ncnc5[nH]4)cc3)C(F)(F)F)CC2)C1] | 583.66 | *** | *** | * |
| | C30H36F3N7O2 | | | | |
| 960 | 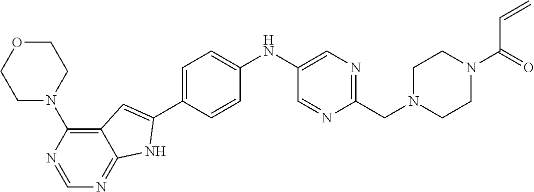 [SMILES: C=CC(=O)N1CCN(Cc2ncc(Nc3ccc(-c4cc5c(N6CCOCC6)ncnc5[nH]4)cc3)cn2)CC1] | 525.62 | *** | *** | * |
| | C28H31N9O2 | | | | |
| 961 | 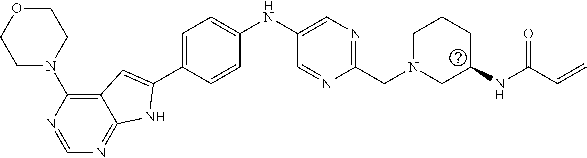 [SMILES: C=CC(=O)N[C@@H]1CCCN(Cc2ncc(Nc3ccc(-c4cc5c(N6CCOCC6)ncnc5[nH]4)cc3)cn2)C1] | 539.64 | ** | *** | * |
| | C29H33N9O2 | | | | |
| 962 | 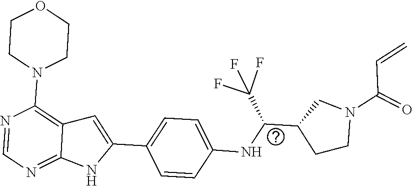 [SMILES: C=CC(=O)N1CC[C@H]([C@H](Nc2ccc(-c3cc4c(N5CCOCC5)ncnc4[nH]3)cc2)C(F)(F)F)C1] | 500.53 | | ** | * |
| | C25H27F3N6O2 | | | | |
| 963 | 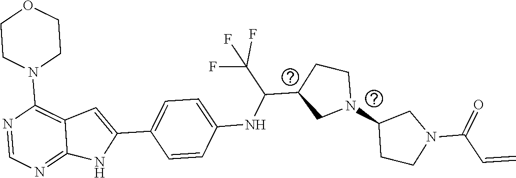 [SMILES: C=CC(=O)N1CC[C@@H](N2CC[C@H](C(Nc3ccc(-c4cc5c(N6CCOCC6)ncnc5[nH]4)cc3)C(F)(F)F)C2)C1] | 569.63 | ** | ** | * |
| | C29H34F3N7O2 | | | | |
| 964 | 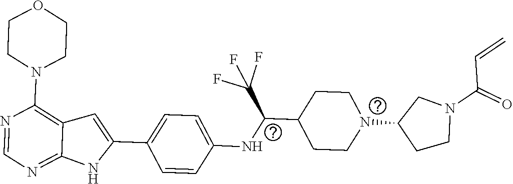 [SMILES: C=CC(=O)N1CC[C@H](N2CCC([C@@H](Nc3ccc(-c4cc5c(N6CCOCC6)ncnc5[nH]4)cc3)C(F)(F)F)CC2)C1] | 583.66 | *** | *** | * |
| | C30H36F3N7O2 | | | | |
| 965 | 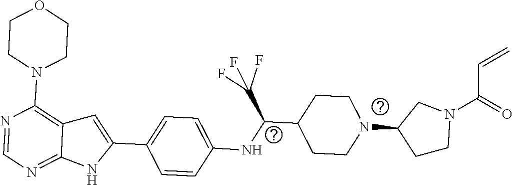 [SMILES: C=CC(=O)N1CC[C@@H](N2CCC([C@@H](Nc3ccc(-c4cc5c(N6CCOCC6)ncnc5[nH]4)cc3)C(F)(F)F)CC2)C1] | 583.66 | ** | *** | * |
| | C30H36F3N7O2 | | | | |
| 966 | 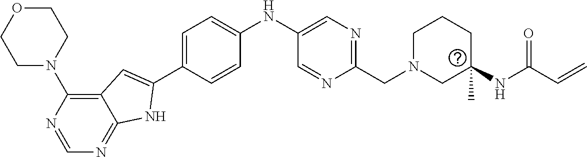 [SMILES: C=CC(=O)N[C@]1(C)CCCN(Cc2ncc(Nc3ccc(-c4cc5c(N6CCOCC6)ncnc5[nH]4)cc3)cn2)C1] | 553.67 | | ** | * |
| | C30H35N9O2 | | | | |
| 967 | 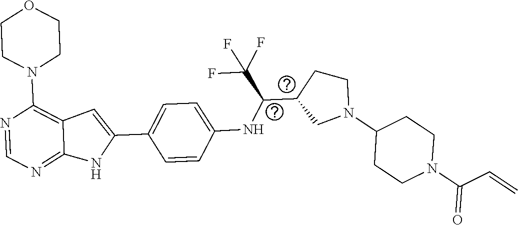 [SMILES: C=CC(=O)N1CCC(N2CC[C@@H]([C@@H](Nc3ccc(-c4cc5c(N6CCOCC6)ncnc5[nH]4)cc3)C(F)(F)F)C2)CC1] | 583.66 | *** | *** | * |
| | C30H36F3N7O2 | | | | |
| 968 | 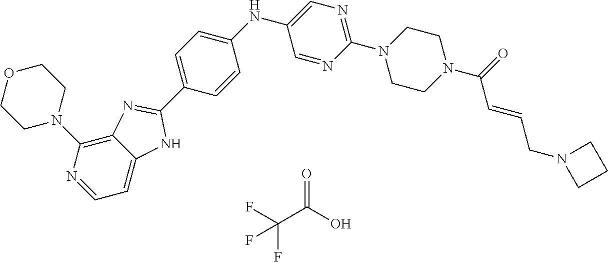 [SMILES: O=C(/C=C/CN1CCC1)N1CCN(c2ncc(Nc3ccc(-c4nc5c(N6CCOCC6)nccc5[nH]4)cc3)cn2)CC1 ; O=C(O)C(F)(F)F] | 694.72 | | * | * |
| | C33H37F3N10O4 | | | | |
| 969 | 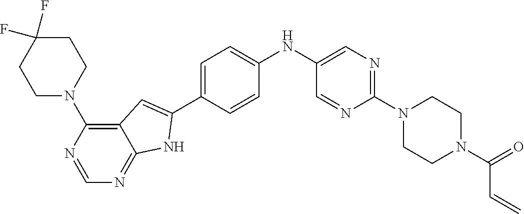 [SMILES: C=CC(=O)N1CCN(c2ncc(Nc3ccc(-c4cc5c(N6CCC(F)(F)CC6)ncnc5[nH]4)cc3)cn2)CC1] | 545.6 | *** | *** | * |
| | C28H29F2N9O | | | | |
| 970 | 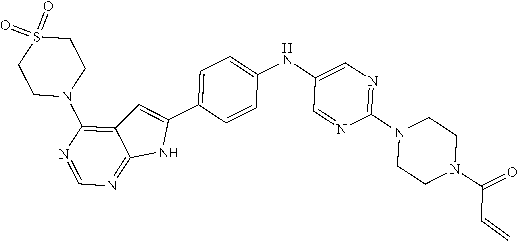 [SMILES: C=CC(=O)N1CCN(c2ncc(Nc3ccc(-c4cc5c(N6CCS(=O)(=O)CC6)ncnc5[nH]4)cc3)cn2)CC1] | 559.65 | *** | *** | * |
| | C27H29N9O3S | | | | |
| 971 | 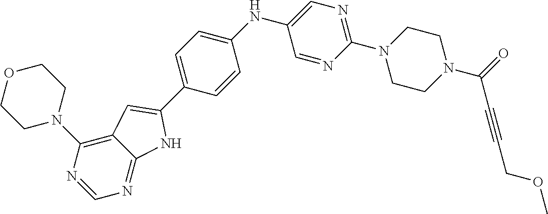 [SMILES: COCC#CC(=O)N1CCN(c2ncc(Nc3ccc(-c4cc5c(N6CCOCC6)ncnc5[nH]4)cc3)cn2)CC1] | 553.63 | *** | *** | ** |
| | C29H31N9O3 | | | | |
| 972 | 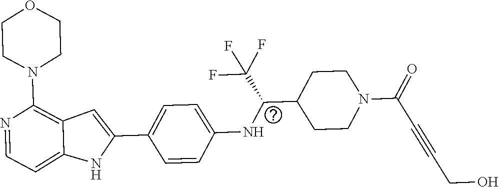 [SMILES: O=C(C#CCO)N1CCC([C@H](Nc2ccc(-c3cc4c(N5CCOCC5)nccc4[nH]3)cc2)C(F)(F)F)CC1] | 541.58 | | ** | * |
| | C28H30F3N5O3 | | | | |
| 973 | 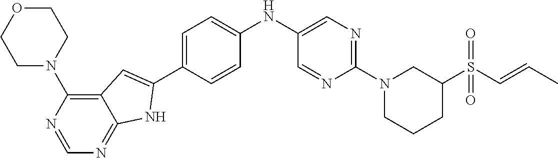 [SMILES: C/C=C/S(=O)(=O)C1CCCN(c2ncc(Nc3ccc(-c4cc5c(N6CCOCC6)ncnc5[nH]4)cc3)cn2)C1] | 560.68 | | ** | * |
| | C28H32N8O3S | | | | |
| 1001 | 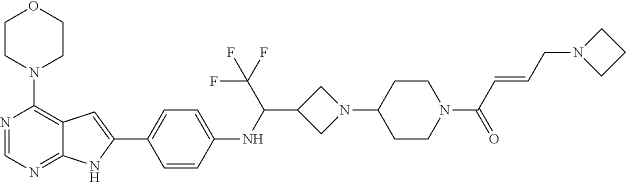 [SMILES: O=C(/C=C/CN1CCC1)N1CCC(N2CC(C(Nc3ccc(-c4cc5c(N6CCOCC6)ncnc5[nH]4)cc3)C(F)(F)F)C2)CC1] | 638.74 | | ** | * |
| | C33H41F3N8O2 | | | | |
| 1002 | 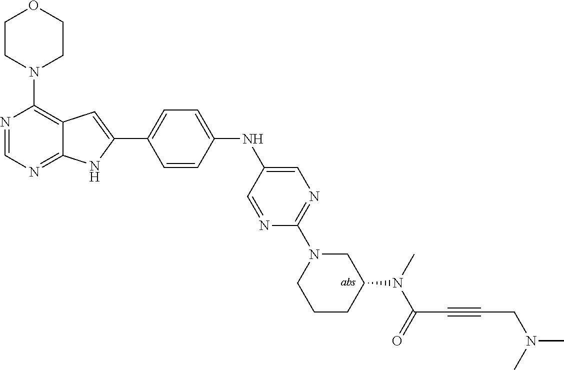 [SMILES: CN(C)CC#CC(=O)N(C)[C@@H]1CCCN(c2ncc(Nc3ccc(-c4cc5c(N6CCOCC6)ncnc5[nH]4)cc3)cn2)C1] | 594.72 | *** | *** | ** |
| | C32H38N10O2 | | | | |
| 1003 | 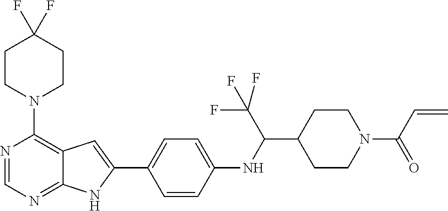 [SMILES: C=CC(=O)N1CCC(C(Nc2ccc(-c3cc4c(N5CCC(F)(F)CC5)ncnc4[nH]3)cc2)C(F)(F)F)CC1] | 548.56 | | ** | * |
| | C27H29F5N6O | | | | |
| 1004 | 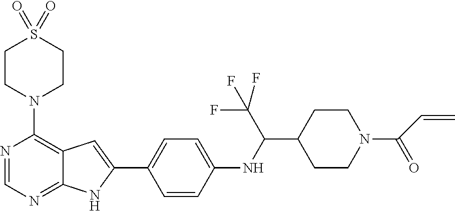 [SMILES: C=CC(=O)N1CCC(C(Nc2ccc(-c3cc4c(N5CCS(=O)(=O)CC5)ncnc4[nH]3)cc2)C(F)(F)F)CC1] | 562.61 | | ** | * |
| | C26H29F3N6O3S | | | | |
| 1005 | 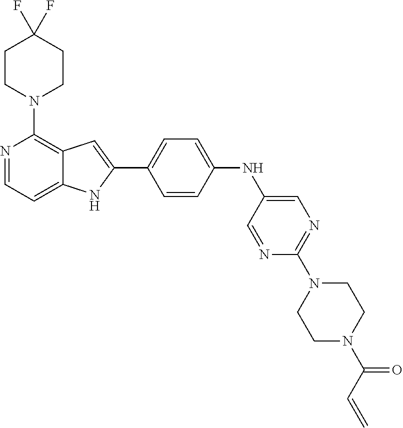 [SMILES: C=CC(=O)N1CCN(c2ncc(Nc3ccc(-c4cc5c(N6CCC(F)(F)CC6)nccc5[nH]4)cc3)cn2)CC1] | 544.61 | | * | * |
| | C29H30F2N8O | | | | |
| 1006 | 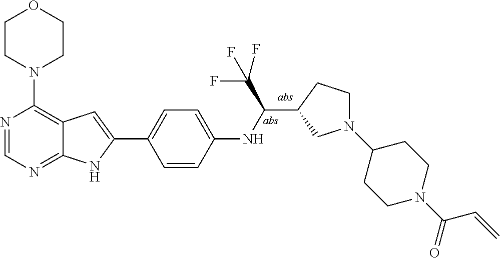 [SMILES: C=CC(=O)N1CCC(N2CC[C@@H]([C@@H](Nc3ccc(-c4cc5c(N6CCOCC6)ncnc5[nH]4)cc3)C(F)(F)F)C2)CC1] | 583.66 | *** | *** | * |
| | C30H36F3N7O2 | | | | |
| 1007 | 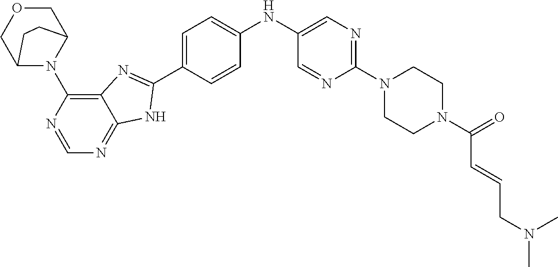 [SMILES: CN(C)C/C=C/C(=O)N1CCN(c2ncc(Nc3ccc(-c4nc5c(N6C7CCC6COC7)ncnc5[nH]4)cc3)cn2)CC1] | 594.72 | | * | * |
| | C32H38N10O2 | | | | |
| 1008 | 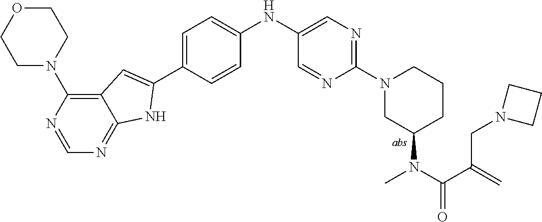 [SMILES: C=C(CN1CCC1)C(=O)N(C)[C@@H]1CCCN(c2ncc(Nc3ccc(-c4cc5c(N6CCOCC6)ncnc5[nH]4)cc3)cn2)C1] | 608.75 | | ** | * |
| | C33H40N10O2 | | | | |
| 1009 | 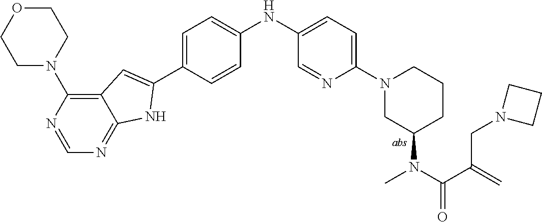 [SMILES: C=C(CN1CCC1)C(=O)N(C)[C@@H]1CCCN(c2ccc(Nc3ccc(-c4cc5c(N6CCOCC6)ncnc5[nH]4)cc3)cn2)C1] | 607.76 | | ** | * |
| | C34H41N9O2 | | | | |
| 1010 | 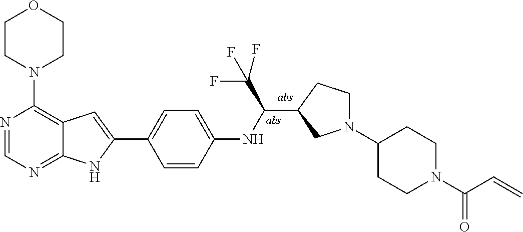 [SMILES: C=CC(=O)N1CCC(N2CC[C@H]([C@@H](Nc3ccc(-c4cc5c(N6CCOCC6)ncnc5[nH]4)cc3)C(F)(F)F)C2)CC1] | 583.66 | *** | *** | * |
| | C30H36F3N7O2 | | | | |
| 1011 | 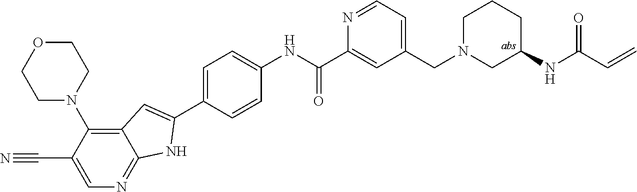 [SMILES: C=CC(=O)N[C@@H]1CCCN(Cc2ccnc(C(=O)Nc3ccc(-c4cc5c(N6CCOCC6)c(C#N)cnc5[nH]4)cc3)c2)C1] | 590.69 | | ** | * |
| | C33H34N8O3 | | | | |
| 1012 | 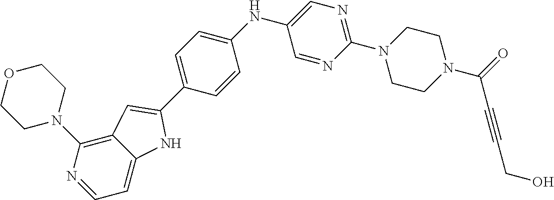 [SMILES: O=C(C#CCO)N1CCN(c2ncc(Nc3ccc(-c4cc5c(N6CCOCC6)nccc5[nH]4)cc3)cn2)CC1] | 538.61 | | ** | * |
| | C29H30N8O3 | | | | |
| 1013 | 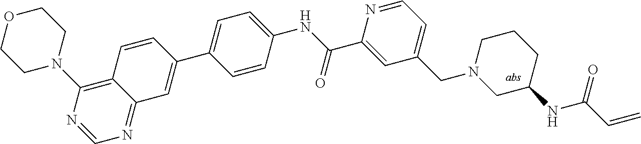 [SMILES: C=CC(=O)N[C@@H]1CCCN(Cc2ccnc(C(=O)Nc3ccc(-c4ccc5c(N6CCOCC6)ncnc5c4)cc3)c2)C1] | 577.69 | | * | * |
| | C33H35N7O3 | | | | |
| 1014 | 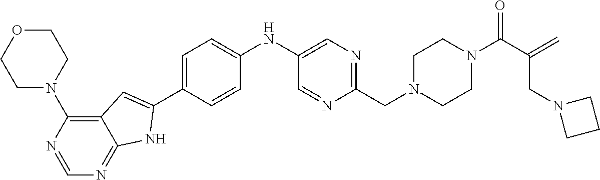 [SMILES: C=C(CN1CCC1)C(=O)N1CCN(Cc2ncc(Nc3ccc(-c4cc5c(N6CCOCC6)ncnc5[nH]4)cc3)cn2)CC1] | 594.72 | | ** | * |
| | C32H38N10O2 | | | | |
| 1015 | 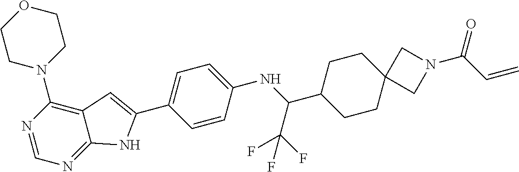 [SMILES: C=CC(=O)N1CC2(CCC(C(Nc3ccc(-c4cc5c(N6CCOCC6)ncnc5[nH]4)cc3)C(F)(F)F)CC2)C1] | 554.62 | | ** | * |
| | C29H33F3N6O2 | | | | |
| 1016 | 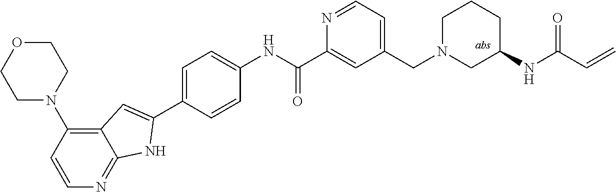 [SMILES: C=CC(=O)N[C@@H]1CCCN(Cc2ccnc(C(=O)Nc3ccc(-c4cc5c(N6CCOCC6)ccnc5[nH]4)cc3)c2)C1] | 565.68 | | ** | * |
| | C32H35N7O3 | | | | |
| 1017 | 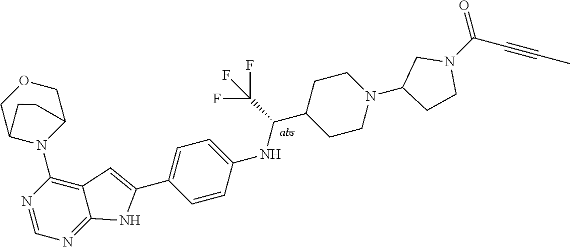 [SMILES: CC#CC(=O)N1CCC(N2CCC([C@H](Nc3ccc(-c4cc5c(N6C7CCC6COC7)ncnc5[nH]4)cc3)C(F)(F)F)CC2)C1] | 621.71 | | ** | * |
| | C33H38F3N7O2 | | | | |
| 1018 | 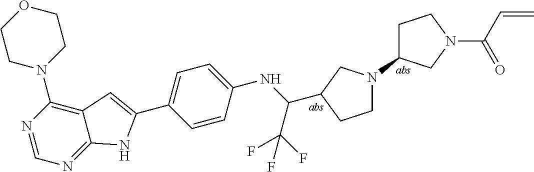 [SMILES: C=CC(=O)N1CC[C@H](N2CCC(C(Nc3ccc(-c4cc5c(N6CCOCC6)ncnc5[nH]4)cc3)C(F)(F)F)C2)C1] | 569.63 | ** | *** | * |
| | C29H34F3N7O2 | | | | |
| 1019 | 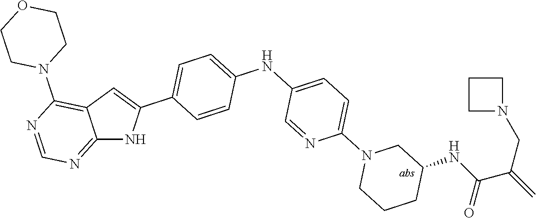 [SMILES: C=C(CN1CCC1)C(=O)N[C@@H]1CCCN(c2ccc(Nc3ccc(-c4cc5c(N6CCOCC6)ncnc5[nH]4)cc3)cn2)C1] | 593.74 | *** | *** | ** |
| | C33H39N9O2 | | | | |
| 1020 | 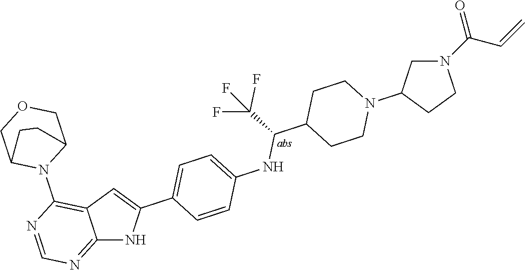 [SMILES: C=CC(=O)N1CCC(N2CCC([C@H](Nc3ccc(-c4cc5c(N6C7CCC6COC7)ncnc5[nH]4)cc3)C(F)(F)F)CC2)C1] | 609.7 | ** | *** | * |
| | C32H38F3N7O2 | | | | |
| 1021 | 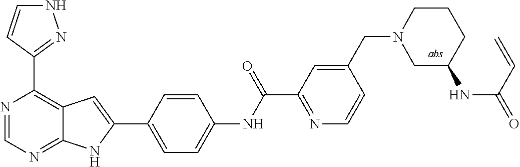 [SMILES: C=CC(=O)N[C@@H]1CCCN(Cc2ccnc(C(=O)Nc3ccc(-c4cc5c(-c6cc[nH]n6)ncnc5[nH]4)cc3)c2)C1] | 547.62 | | * | * |
| | C30H29N9O2 | | | | |
| 1022 | 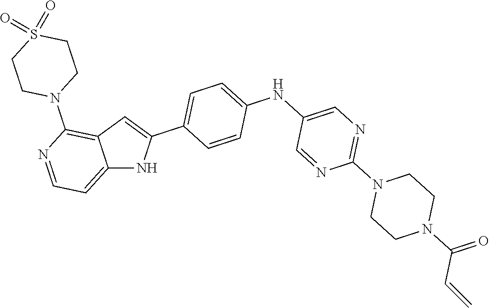 [SMILES: C=CC(=O)N1CCN(c2ncc(Nc3ccc(-c4cc5c(N6CCS(=O)(=O)CC6)nccc5[nH]4)cc3)cn2)CC1] | 558.66 | | * | * |
| | C28H30N8O3S | | | | |
| 1023 | 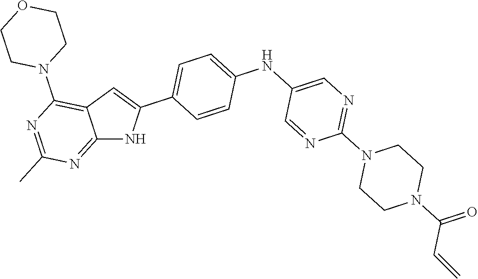 [SMILES: C=CC(=O)N1CCN(c2ncc(Nc3ccc(-c4cc5c(N6CCOCC6)nc(C)nc5[nH]4)cc3)cn2)CC1] | 525.62 | | * | * |
| | C28H31N9O2 | | | | |
| 1024 | 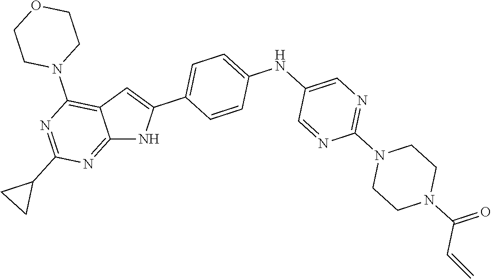 [SMILES: C=CC(=O)N1CCN(c2ncc(Nc3ccc(-c4cc5c(N6CCOCC6)nc(C6CC6)nc5[nH]4)cc3)cn2)CC1] | 551.65 | | * | * |
| | C30H33N9O2 | | | | |
| 1026 | 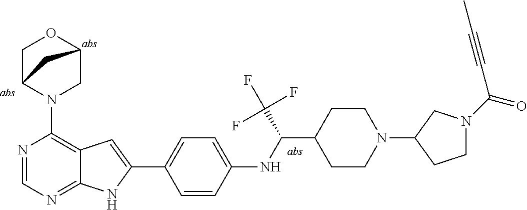 [SMILES: CC#CC(=O)N1CCC(N2CCC([C@H](Nc3ccc(-c4cc5c(N6C[C@@H]7C[C@H]6CO7)ncnc5[nH]4)cc3)C(F)(F)F)CC2)C1] | 607.68 | | * | * |
| | C32H36F3N7O2 | | | | |
| 1027 | 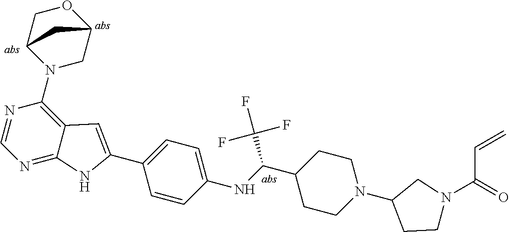 [SMILES: C=CC(=O)N1CCC(N2CCC([C@H](Nc3ccc(-c4cc5c(N6C[C@@H]7C[C@H]6CO7)ncnc5[nH]4)cc3)C(F)(F)F)CC2)C1] | 595.67 | | ** | * |
| | C31H36F3N7O2 | | | | |
| 1028 | 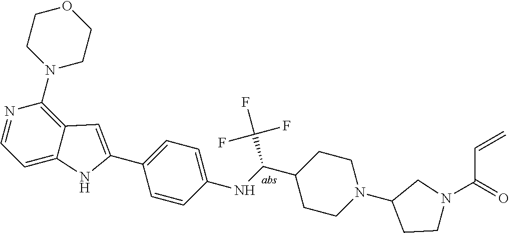 [SMILES: C=CC(=O)N1CCC(N2CCC([C@H](Nc3ccc(-c4cc5c(N6CCOCC6)nccc5[nH]4)cc3)C(F)(F)F)CC2)C1] | 582.67 | | * | * |
| | C31H37F3N6O2 | | | | |
| 1029 | 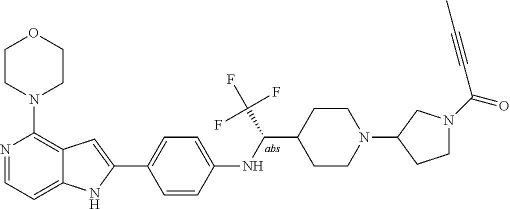 [SMILES: CC#CC(=O)N1CCC(N2CCC([C@H](Nc3ccc(-c4cc5c(N6CCOCC6)nccc5[nH]4)cc3)C(F)(F)F)CC2)C1] | 594.68 | | * | * |
| | C32H37F3N6O2 | | | | |
| 1031 | 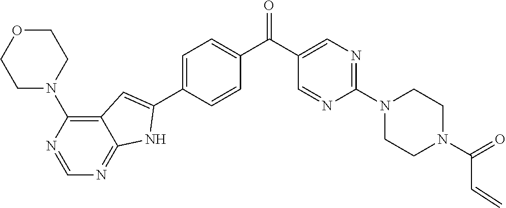 [SMILES: C=CC(=O)N1CCN(c2ncc(C(=O)c3ccc(-c4cc5c(N6CCOCC6)ncnc5[nH]4)cc3)cn2)CC1] | 524.58 | | ** | * |
| | C28H28N8O3 | | | | |
| 1032 | 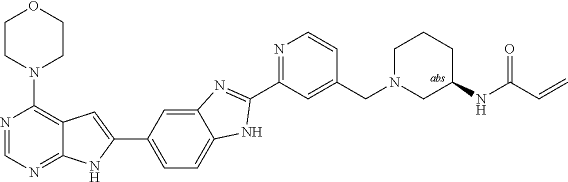 [SMILES: C=CC(=O)N[C@@H]1CCCN(Cc2ccnc(-c3nc4cc(-c5cc6c(N7CCOCC7)ncnc6[nH]5)ccc4[nH]3)c2)C1] | 563.67 | | * | * |
| | C31H33N9O2 | | | | |
| 1033 | 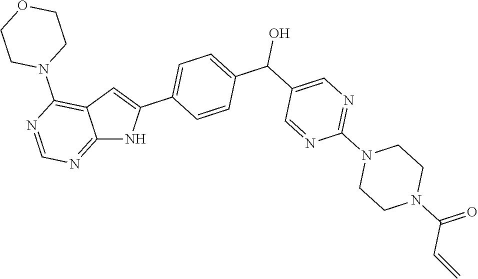 [SMILES: C=CC(=O)N1CCN(c2ncc(C(O)c3ccc(-c4cc5c(N6CCOCC6)ncnc5[nH]4)cc3)cn2)CC1] | 526.6 | *** | *** | ** |
| | C28H30N8O3 | | | | |
| 1034 | 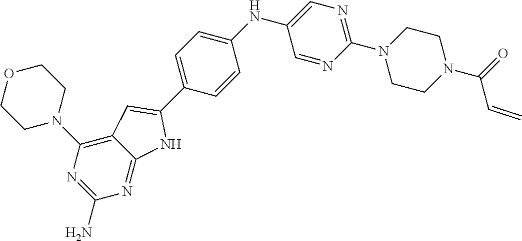 [SMILES: C=CC(=O)N1CCN(c2ncc(Nc3ccc(-c4cc5c(N6CCOCC6)nc(N)nc5[nH]4)cc3)cn2)CC1] | 526.6 | | ** | * |
| | C27H30N10O2 | | | | |
| 1035 | 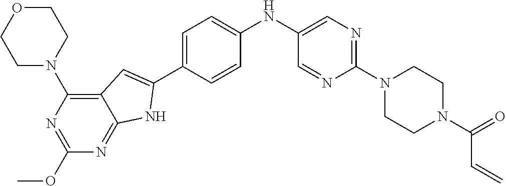 [SMILES: C=CC(=O)N1CCN(c2ncc(Nc3ccc(-c4cc5c(N6CCOCC6)nc(OC)nc5[nH]4)cc3)cn2)CC1] | 541.62 | | * | * |
| | C28H31N9O3 | | | | |
| 1036 | 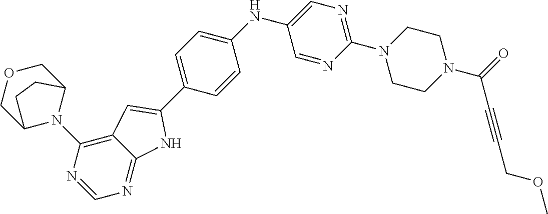 [SMILES: COCC#CC(=O)N1CCN(c2ncc(Nc3ccc(-c4cc5c(N6C7CCC6COC7)ncnc5[nH]4)cc3)cn2)CC1] | 579.66 | *** | *** | ** |
| | C31H33N9O3 | | | | |
| 1037 | 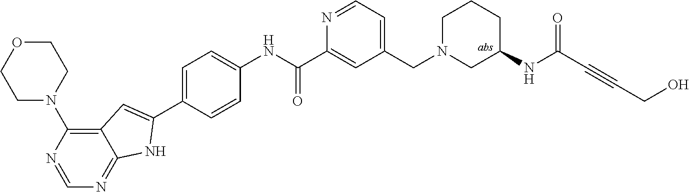 [SMILES: O=C(C#CCO)N[C@@H]1CCCN(Cc2ccnc(C(=O)Nc3ccc(-c4cc5c(N6CCOCC6)ncnc5[nH]4)cc3)c2)C1] | 594.68 | *** | *** | *** |
| | C32H34N8O4 | | | | |
| 1038 | 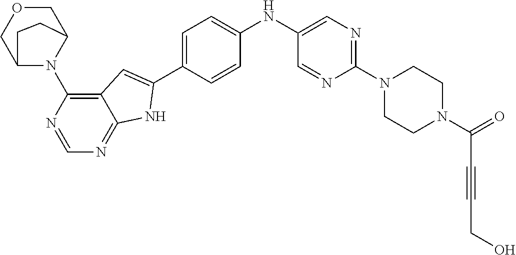 [SMILES: O=C(C#CCO)N1CCN(c2ncc(Nc3ccc(-c4cc5c(N6C7CCC6COC7)ncnc5[nH]4)cc3)cn2)CC1] | 565.64 | ** | *** | * |
| | C30H31N9O3 | | | | |
| 1039 | 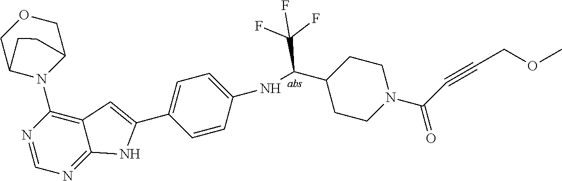 [SMILES: COCC#CC(=O)N1CCC([C@@H](Nc2ccc(-c3cc4c(N5C6CCC5COC6)ncnc4[nH]3)cc2)C(F)(F)F)CC1] | 582.63 | | ** | * |
| | C30H33F3N6O3 | | | | |
| 1040 | 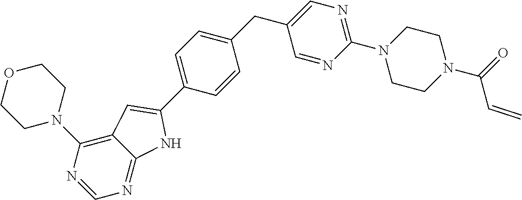 [SMILES: C=CC(=O)N1CCN(c2ncc(Cc3ccc(-c4cc5c(N6CCOCC6)ncnc5[nH]4)cc3)cn2)CC1] | 510.6 | ** | *** | * |
| | C28H30N8O2 | | | | |
| 1041 | 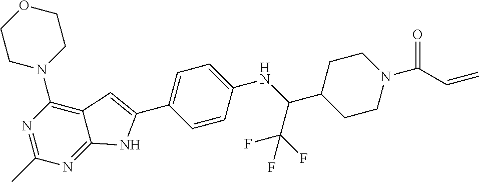 [SMILES: C=CC(=O)N1CCC(C(Nc2ccc(-c3cc4c(N5CCOCC5)nc(C)nc4[nH]3)cc2)C(F)(F)F)CC1] | 528.58 | | * | * |
| | C27H31F3N6O2 | | | | |
| 1042 | 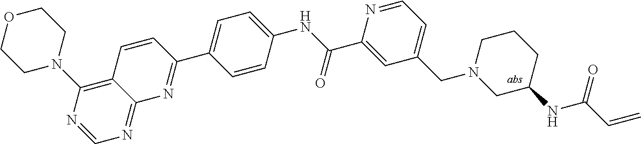 [SMILES: C=CC(=O)N[C@@H]1CCCN(Cc2ccnc(C(=O)Nc3ccc(-c4ccc5c(N6CCOCC6)ncnc5n4)cc3)c2)C1] | 578.68 | | * | * |
| | C32H34N8O3 | | | | |
| 1044 | 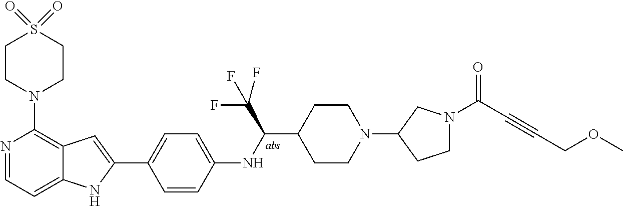 [SMILES: COCC#CC(=O)N1CCC(N2CCC([C@@H](Nc3ccc(-c4cc5c(N6CCS(=O)(=O)CC6)nccc5[nH]4)cc3)C(F)(F)F)CC2)C1] | 672.77 | | ** | * |
| | C33H39F3N6O4S | | | | |
| 1045 | 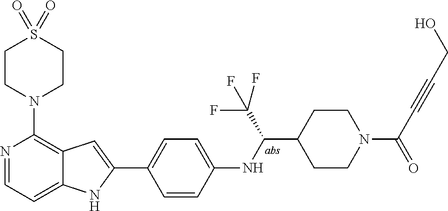 [SMILES: O=C(C#CCO)N1CCC([C@H](Nc2ccc(-c3cc4c(N5CCS(=O)(=O)CC5)nccc4[nH]3)cc2)C(F)(F)F)CC1] | 589.63 | | * | * |
| | C28H30F3N5O4S | | | | |
| 1046 | 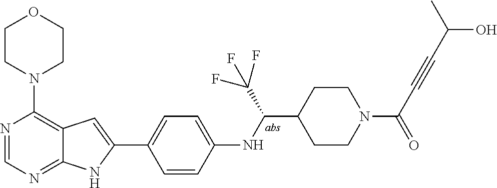 [SMILES: CC(O)C#CC(=O)N1CCC([C@H](Nc2ccc(-c3cc4c(N5CCOCC5)ncnc4[nH]3)cc2)C(F)(F)F)CC1] | 556.59 | | ** | * |
| | C28H31F3N6O3 | | | | |
| 1047 | 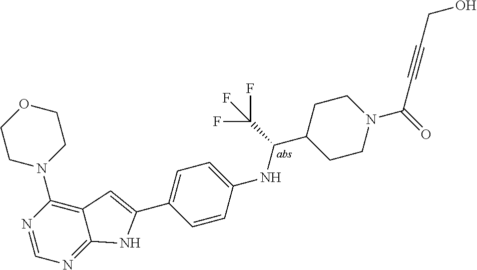 [SMILES: O=C(C#CCO)N1CCC([C@H](Nc2ccc(-c3cc4c(N5CCOCC5)ncnc4[nH]3)cc2)C(F)(F)F)CC1] | 542.56 | | ** | * |
| | C27H29F3N6O3 | | | | |
| 1048 | 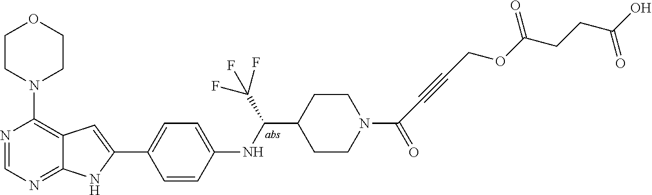 [SMILES: O=C(O)CCC(=O)OCC#CC(=O)N1CCC([C@H](Nc2ccc(-c3cc4c(N5CCOCC5)ncnc4[nH]3)cc2)C(F)(F)F)CC1] | 642.64 | | ** | * |
| | C31H33F3N6O6 | | | | |
| 1049 | 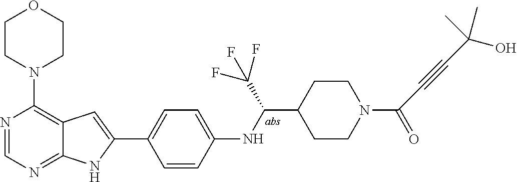 [SMILES: CC(C)(O)C#CC(=O)N1CCC([C@H](Nc2ccc(-c3cc4c(N5CCOCC5)ncnc4[nH]3)cc2)C(F)(F)F)CC1] | 570.62 | | ** | * |
| | C29H33F3N6O3 | | | | |
| 1050 | 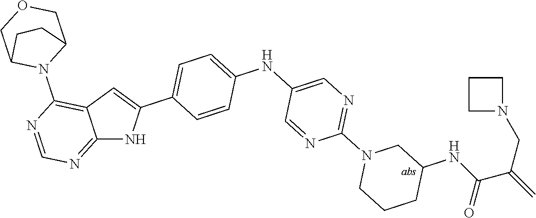 [SMILES: C=C(CN1CCC1)C(=O)NC1CCCN(c2ncc(Nc3ccc(-c4cc5c(N6C7CCC6COC7)ncnc5[nH]4)cc3)cn2)C1] | 620.76 | ** | *** | * |
| | C34H40N10O2 | | | | |
| 1051 | 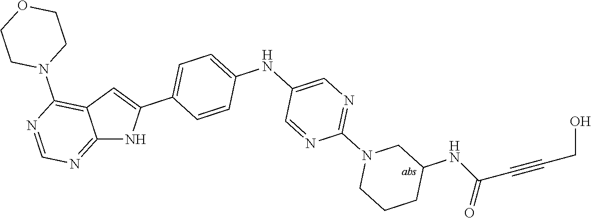 [SMILES: O=C(C#CCO)NC1CCCN(c2ncc(Nc3ccc(-c4cc5c(N6CCOCC6)ncnc5[nH]4)cc3)cn2)C1] | 553.63 | *** | *** | * |
| | C29H31N9O3 | | | | |
| 1052 | 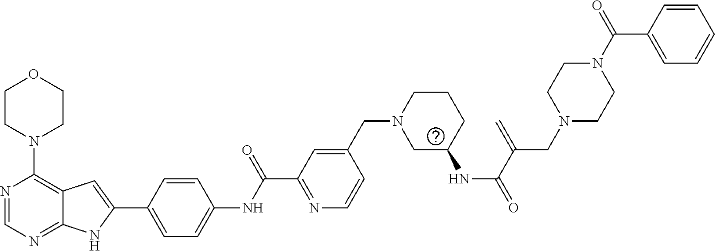 [SMILES: C=C(CN1CCN(C(=O)c2ccccc2)CC1)C(=O)N[C@@H]1CCCN(Cc2ccnc(C(=O)Nc3ccc(-c4cc5c(N6CCOCC6)ncnc5[nH]4)cc3)c2)C1] | 768.92 | | ** | ** |
| | C43H48N10O4 | | | | |
| 1053 | 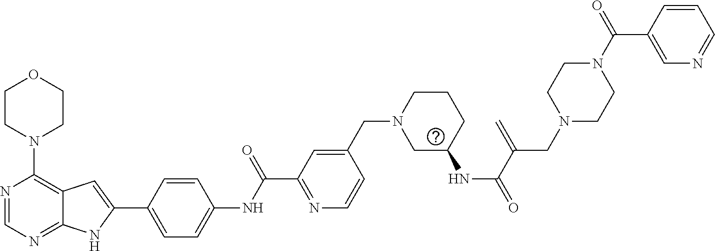 [SMILES: C=C(CN1CCN(C(=O)c2cccnc2)CC1)C(=O)N[C@@H]1CCCN(Cc2ccnc(C(=O)Nc3ccc(-c4cc5c(N6CCOCC6)ncnc5[nH]4)cc3)c2)C1] | 769.91 | *** | *** | ** |
| | C42H47N11O4 | | | | |
| 1054 | 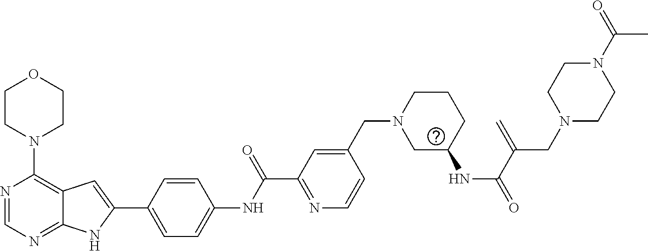 [SMILES: C=C(CN1CCN(C(C)=O)CC1)C(=O)N[C@@H]1CCCN(Cc2ccnc(C(=O)Nc3ccc(-c4cc5c(N6CCOCC6)ncnc5[nH]4)cc3)c2)C1] | 706.85 | *** | *** | *** |
| | C38H46N10O4 | | | | |
| 1055 | 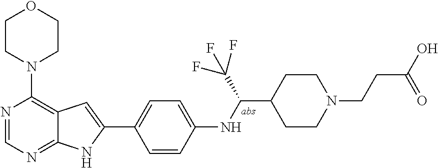 [SMILES: O=C(O)CCN1CCC([C@H](Nc2ccc(-c3cc4c(N5CCOCC5)ncnc4[nH]3)cc2)C(F)(F)F)CC1] | 532.57 | | * | * |
| | C26H31F3N6O3 | | | | |
| 1056 | 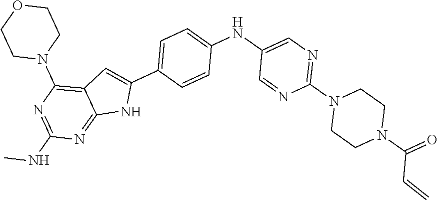 [SMILES: C=CC(=O)N1CCN(c2ncc(Nc3ccc(-c4cc5c(N6CCOCC6)nc(NC)nc5[nH]4)cc3)cn2)CC1] | 540.63 | | ** | * |
| | C28H32N10O2 | | | | |
| 1057 | 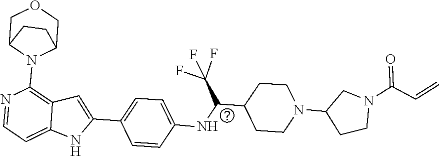 | 608.71 | | * | * |
| | C33H39F3N6O2 | | | | |
| 1058 | 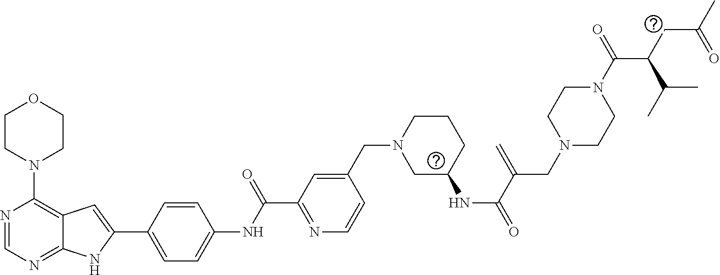 [SMILES: C=C(CN1CCN(C(=O)[C@@H](CC(C)=O)C(C)C)CC1)C(=O)N[C@@H]1CCCN(Cc2ccnc(C(=O)Nc3ccc(-c4cc5c(N6CCOCC6)ncnc5[nH]4)cc3)c2)C1] | 805.98 | *** | *** | ** |
| | C43H55N11O5 | | | | |
| 1059 | 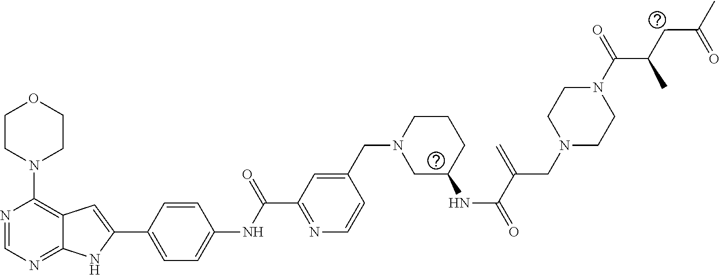 [SMILES: C=C(CN1CCN(C(=O)[C@H](C)CC(C)=O)CC1)C(=O)N[C@@H]1CCCN(Cc2ccnc(C(=O)Nc3ccc(-c4cc5c(N6CCOCC6)ncnc5[nH]4)cc3)c2)C1] | 777.93 | *** | *** | ** |
| | C41H51N11O5 | | | | |
| 1060 | 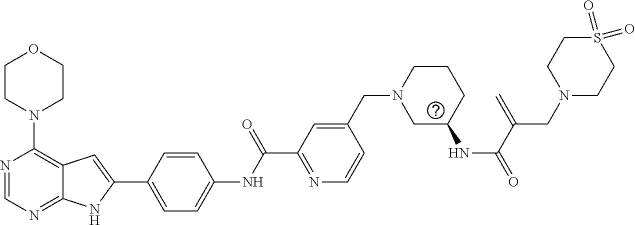 [SMILES: C=C(CN1CCS(=O)(=O)CC1)C(=O)N[C@@H]1CCCN(Cc2ccnc(C(=O)Nc3ccc(-c4cc5c(N6CCOCC6)ncnc5[nH]4)cc3)c2)C1] | 713.86 | | * | * |
| | C36H43N9O5S | | | | |
| 1061 | 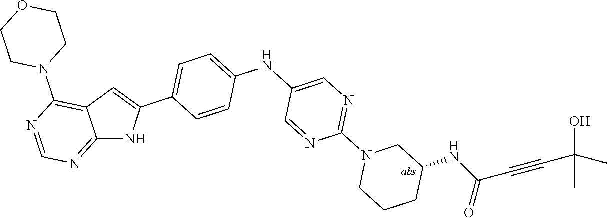 [SMILES: CC(C)(O)C#CC(=O)N[C@@H]1CCCN(c2ncc(Nc3ccc(-c4cc5c(N6CCOCC6)ncnc5[nH]4)cc3)cn2)C1] | 581.68 | | ** | * |
| | C31H35N9O3 | | | | |
| 1062 | 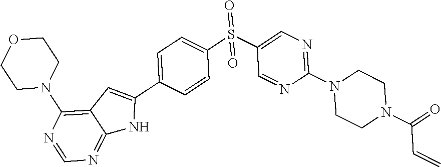 [SMILES: C=CC(=O)N1CCN(c2ncc(S(=O)(=O)c3ccc(-c4cc5c(N6CCOCC6)ncnc5[nH]4)cc3)cn2)CC1] | 560.63 | *** | *** | ** |
| | C27H28N8O4S | | | | |
| 1064 | 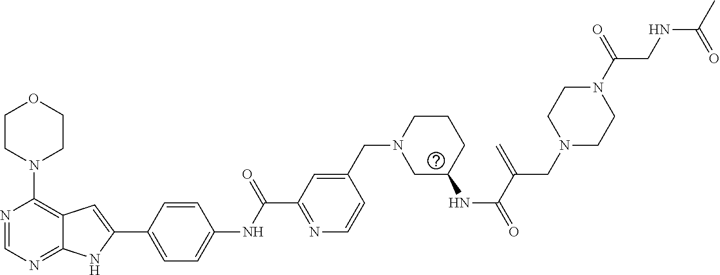 [SMILES: C=C(CN1CCN(C(=O)CNC(C)=O)CC1)C(=O)N[C@@H]1CCCN(Cc2ccnc(C(=O)Nc3ccc(-c4cc5c(N6CCOCC6)ncnc5[nH]4)cc3)c2)C1] | 763.9 | *** | *** | ** |
| | C40H49N11O5 | | | | |
| 1065 | 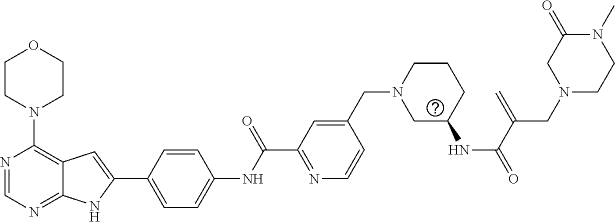 [SMILES: C=C(CN1CCN(C)C(=O)C1)C(=O)N[C@@H]1CCCN(Cc2ccnc(C(=O)Nc3ccc(-c4cc5c(N6CCOCC6)ncnc5[nH]4)cc3)c2)C1] | 692.82 | | ** | * |
| | C37H44N10O4 | | | | |
| 1066 | 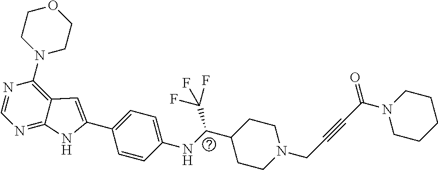 [SMILES: O=C(C#CCN1CCC([C@H](Nc2ccc(-c3cc4c(N5CCOCC5)ncnc4[nH]3)cc2)C(F)(F)F)CC1)N1CCCCC1] | 597.69 | | ** | * |
| | C31H38F3N7O2 | | | | |
| 1067 | 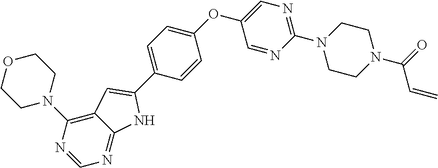 [SMILES: C=CC(=O)N1CCN(c2ncc(Oc3ccc(-c4cc5c(N6CCOCC6)ncnc5[nH]4)cc3)cn2)CC1] | 512.57 | *** | *** | * |
| | C27H28N8O3 | | | | |
| 1068 | 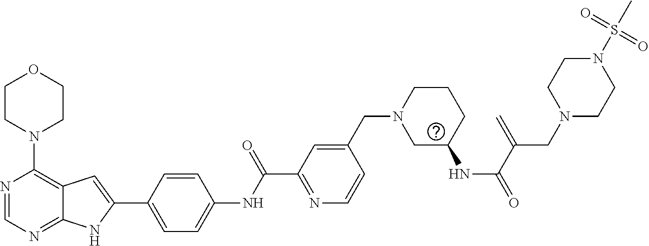 [SMILES: C=C(CN1CCN(S(C)(=O)=O)CC1)C(=O)N[C@@H]1CCCN(Cc2ccnc(C(=O)Nc3ccc(-c4cc5c(N6CCOCC6)ncnc5[nH]4)cc3)c2)C1] | 742.9 | *** | *** | ** |
| | C37H46N10O5S | | | | |
| 1069 | 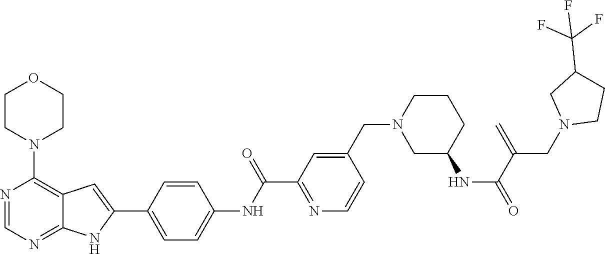 [SMILES: C=C(CN1CCC(C(F)(F)F)C1)C(=O)N[C@@H]1CCCN(Cc2ccnc(C(=O)Nc3ccc(-c4cc5c(N6CCOCC6)ncnc5[nH]4)cc3)c2)C1] | 717.8 | *** | *** | ** |
| | C37H42F3N9O3 | | | | |
| 1070 | 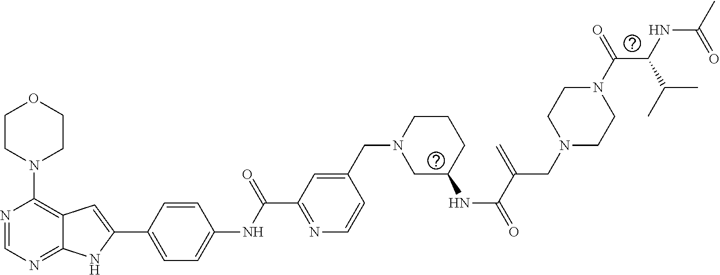 [SMILES: C=C(CN1CCN(C(=O)[C@H](NC(C)=O)C(C)C)CC1)C(=O)N[C@@H]1CCCN(Cc2ccnc(C(=O)Nc3ccc(-c4cc5c(N6CCOCC6)ncnc5[nH]4)cc3)c2)C1] | 805.98 | *** | *** | ** |
| | C43H55N11O5 | | | | |
| 1071 | 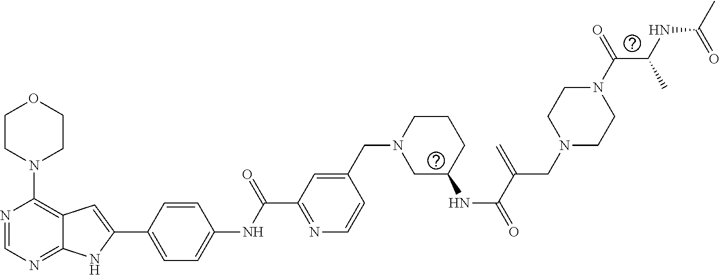 | 777.93 | *** | *** | ** |
| | C41H51N11O5 | | | | |
| 1072 | 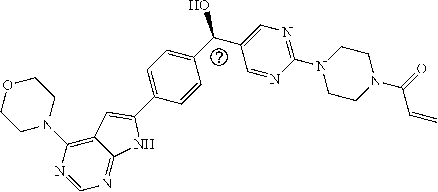 [SMILES: C=CC(=O)N1CCN(c2ncc([C@H](O)c3ccc(-c4cc5c(N6CCOCC6)ncnc5[nH]4)cc3)cn2)CC1] | 526.6 | *** | *** | *** |
| | C28H30N8O3 | | | | |
| 1073 | 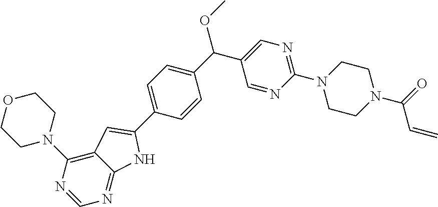 [SMILES: C=CC(=O)N1CCN(c2ncc(C(OC)c3ccc(-c4cc5c(N6CCOCC6)ncnc5[nH]4)cc3)cn2)CC1] | 540.63 | *** | *** | * |
| | C29H32N8O3 | | | | |
| 1074 | 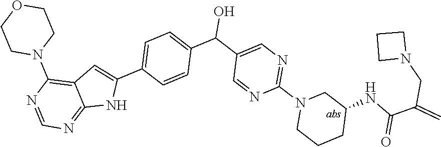 [SMILES: C=C(CN1CCC1)C(=O)N[C@@H]1CCCN(c2ncc(C(O)c3ccc(-c4cc5c(N6CCOCC6)ncnc5[nH]4)cc3)cn2)C1] | 609.73 | *** | *** | ** |
| | C33H39N9O3 | | | | |
| 1075 | 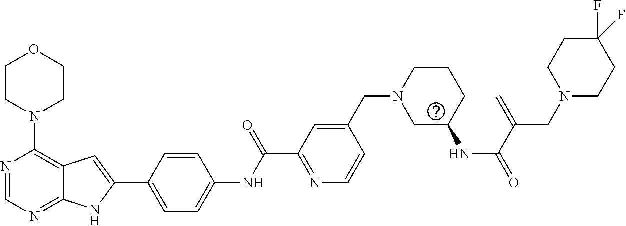 [SMILES: C=C(CN1CCC(F)(F)CC1)C(=O)N[C@@H]1CCCN(Cc2ccnc(C(=O)Nc3ccc(-c4cc5c(N6CCOCC6)ncnc5[nH]4)cc3)c2)C1] | 699.81 | | ** | ** |
| | C37H43F2N9O3 | | | | |
| 1076 | 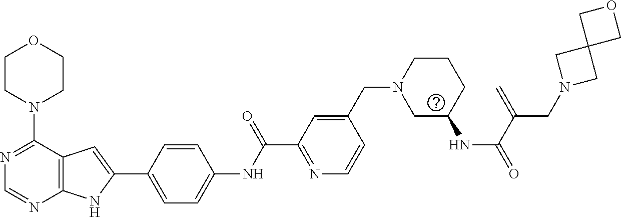 [SMILES: C=C(CN1CC2(COC2)C1)C(=O)N[C@@H]1CCCN(Cc2ccnc(C(=O)Nc3ccc(-c4cc5c(N6CCOCC6)ncnc5[nH]4)cc3)c2)C1] | 677.81 | *** | *** | *** |
| | C37H43N9O4 | | | | |
| 1077 | 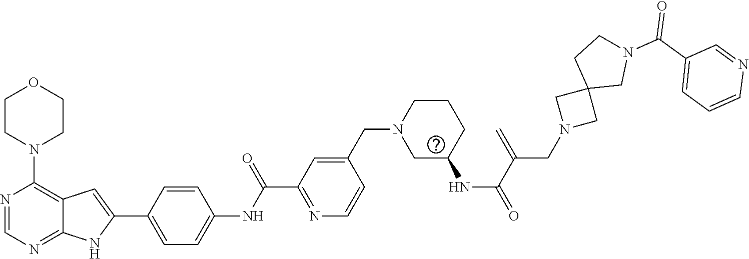 [SMILES: C=C(CN1CC2(CCN(C(=O)c3cccnc3)C2)C1)C(=O)N[C@@H]1CCCN(Cc2ccnc(C(=O)Nc3ccc(-c4cc5c(N6CCOCC6)ncnc5[nH]4)cc3)c2)C1] | 795.95 | *** | *** | ** |
| | C44H49N11O4 | | | | |
| 1078 | 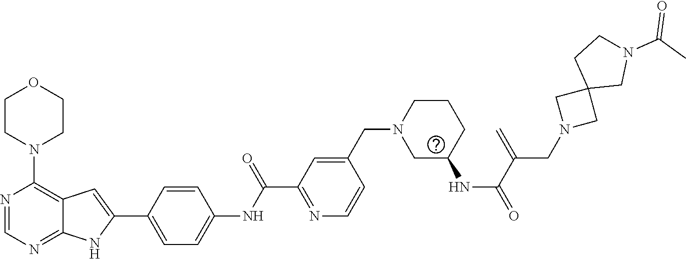 [SMILES: C=C(CN1CC2(CCN(C(C)=O)C2)C1)C(=O)N[C@@H]1CCCN(Cc2ccnc(C(=O)Nc3ccc(-c4cc5c(N6CCOCC6)ncnc5[nH]4)cc3)c2)C1] | 732.89 | *** | *** | *** |
| | C40H48N10O4 | | | | |
| 1079 | 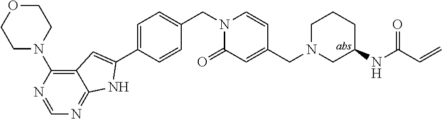 [SMILES: C=CC(=O)N[C@@H]1CCCN(Cc2ccn(Cc3ccc(-c4cc5c(N6CCOCC6)ncnc5[nH]4)cc3)c(=O)c2)C1] | 553.67 | | ** | * |
| | C31H35N7O3 | | | | |
| 1080 | 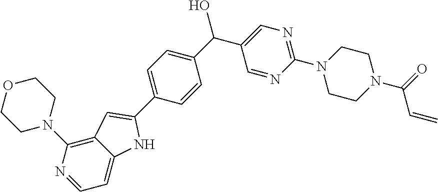 [SMILES: C=CC(=O)N1CCN(c2ncc(C(O)c3ccc(-c4cc5c(N6CCOCC6)nccc5[nH]4)cc3)cn2)CC1] | 525.61 | | * | * |
| | C29H31N7O3 | | | | |
| 1081 | 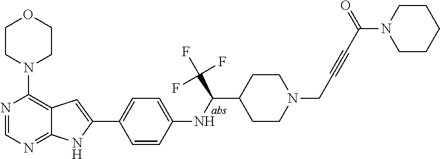 [SMILES: O=C(C#CCN1CCC([C@@H](Nc2ccc(-c3cc4c(N5CCOCC5)ncnc4[nH]3)cc2)C(F)(F)F)CC1)N1CCCCC1] | 597.69 | | ** | ** |
| | C31H38F3N7O2 | | | | |
| 1082 | 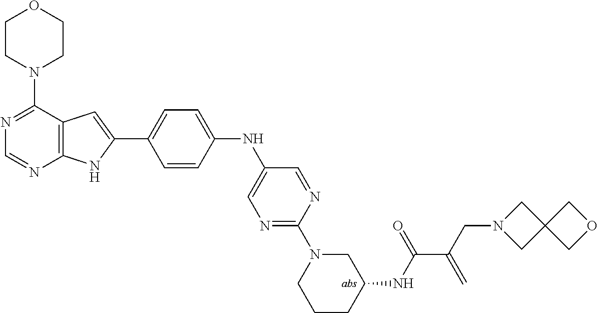 [SMILES: C=C(CN1CC2(COC2)C1)C(=O)N[C@@H]1CCCN(c2ncc(Nc3ccc(-c4cc5c(N6CCOCC6)ncnc5[nH]4)cc3)cn2)C1] | 636.76 | *** | *** | * |
| | C34H40N10O3 | | | | |
| 1083 | 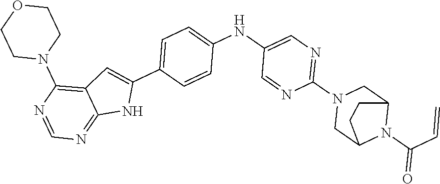 [SMILES: C=CC(=O)N1C2CCC1CN(c1ncc(Nc3ccc(-c4cc5c(N6CCOCC6)ncnc5[nH]4)cc3)cn1)C2] | 537.63 | *** | *** | * |
| | C29H31N9O2 | | | | |
| 1084 | 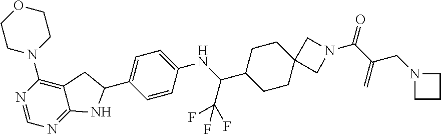 [SMILES: C=C(CN1CCC1)C(=O)N1CC2(CCC(C(Nc3ccc(C4Cc5c(ncnc5N5CCOCC5)N4)cc3)C(F)(F)F)CC2)C1] | 623.73 | *** | *** | * |
| | C33H40F3N7O2 | | | | |
| 1085 | 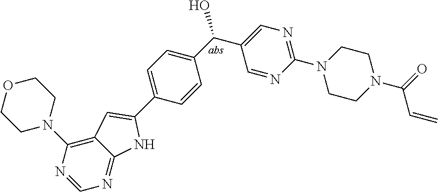 [SMILES: C=CC(=O)N1CCN(c2ncc([C@@H](O)c3ccc(-c4cc5c(N6CCOCC6)ncnc5[nH]4)cc3)cn2)CC1] | 526.6 | *** | *** | ** |
| | C28H30N8O3 | | | | |
| 1086 | 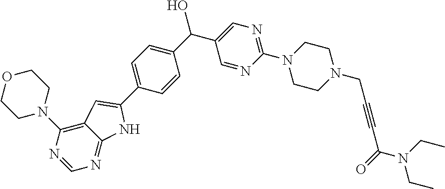 [SMILES: CCN(CC)C(=O)C#CCN1CCN(c2ncc(C(O)c3ccc(-c4cc5c(N6CCOCC6)ncnc5[nH]4)cc3)cn2)CC1] | 609.73 | ** | * | * |
| | C33H39N9O3 | | | | |
| 1087 | 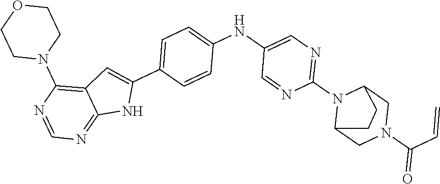 [SMILES: C=CC(=O)N1CC2CCC(C1)N2c1ncc(Nc2ccc(-c3cc4c(N5CCOCC5)ncnc4[nH]3)cc2)cn1] | 537.63 | **** | **** | * |
| | C29H31N9O2 | | | | |
| 1088 | 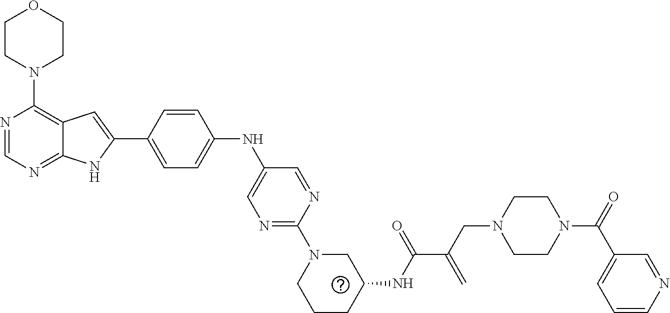 [SMILES: C=C(CN1CCN(C(=O)c2cccnc2)CC1)C(=O)N[C@@H]1CCCN(c2ncc(Nc3ccc(-c4cc5c(N6CCOCC6)ncnc5[nH]4)cc3)cn2)C1] | 728.86 | | ** | * |
| | C39H44N12O3 | | | | |
| 1089 | 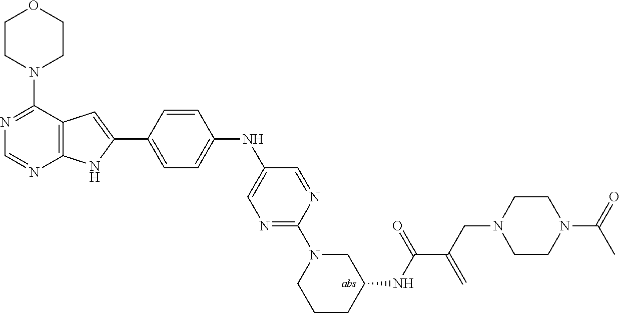 [SMILES: C=C(CN1CCN(C(C)=O)CC1)C(=O)N[C@@H]1CCCN(c2ncc(Nc3ccc(-c4cc5c(N6CCOCC6)ncnc5[nH]4)cc3)cn2)C1] | 665.8 | | ** | * |
| | C35H43N11O3 | | | | |
| 1091 | 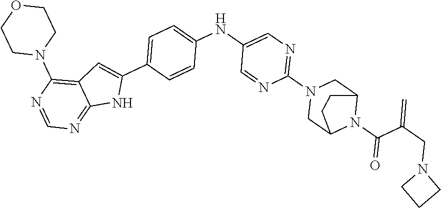 [SMILES: C=C(CN1CCC1)C(=O)N1C2CCC1CN(c1ncc(Nc3ccc(-c4cc5c(N6CCOCC6)ncnc5[nH]4)cc3)cn1)C2] | 606.73 | | ** | * |
| | C33H38N10O2 | | | | |
| 1092 | 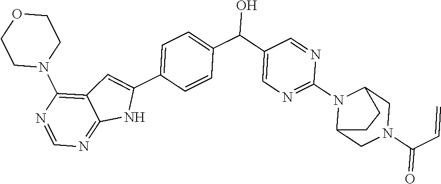 [SMILES: C=CC(=O)N1CC2CCC(C1)N2c1ncc(C(O)c2ccc(-c3cc4c(N5CCOCC5)ncnc4[nH]3)cc2)cn1] | 552.64 | *** | **** | ** |
| | C30H32N8O3 | | | | |
| 1093 | 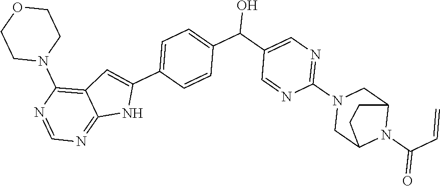 [SMILES: C=CC(=O)N1C2CCC1CN(c1ncc(C(O)c3ccc(-c4cc5c(N6CCOCC6)ncnc5[nH]4)cc3)cn1)C2] | 552.64 | *** | *** | ** |
| | C30H32N8O3 | | | | |
| 1094 | 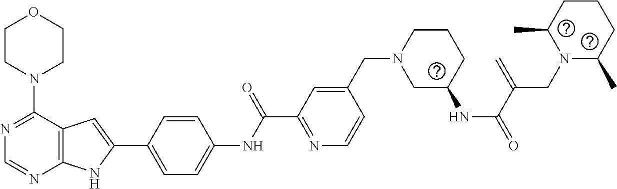 [SMILES: C=C(CN1[C@H](C)CCC[C@@H]1C)C(=O)N[C@@H]1CCCN(Cc2ccnc(C(=O)Nc3ccc(-c4cc5c(N6CCOCC6)ncnc5[nH]4)cc3)c2)C1] | 691.88 | *** | *** | *** |
| | C39H49N9O3 | | | | |
| 1095 | 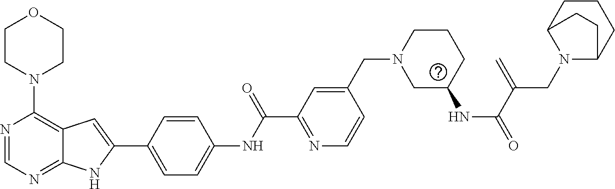 [SMILES: C=C(CN1C2CCCC1CC2)C(=O)N[C@@H]1CCCN(Cc2ccnc(C(=O)Nc3ccc(-c4cc5c(N6CCOCC6)ncnc5[nH]4)cc3)c2)C1] | 689.86 | *** | *** | *** |
| | C39H47N9O3 | | | | |
| 1096 | 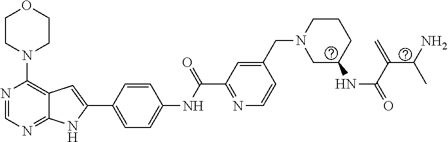 [SMILES: C=C(C(=O)N[C@@H]1CCCN(Cc2ccnc(C(=O)Nc3ccc(-c4cc5c(N6CCOCC6)ncnc5[nH]4)cc3)c2)C1)C(C)N] | 609.73 | ** | ** | * |
| | C33H39N9O3 | | | | |
| 1097 | 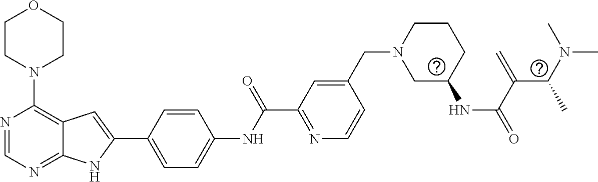 [SMILES: C=C(C(=O)N[C@@H]1CCCN(Cc2ccnc(C(=O)Nc3ccc(-c4cc5c(N6CCOCC6)ncnc5[nH]4)cc3)c2)C1)[C@@H](C)N(C)C] | 637.79 | *** | **** | *** |
| | C35H43N9O3 | | | | |
| 1098 | 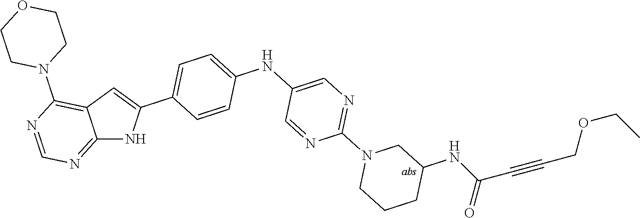 [SMILES: CCOCC#CC(=O)NC1CCCN(c2ncc(Nc3ccc(-c4cc5c(N6CCOCC6)ncnc5[nH]4)cc3)cn2)C1] | 581.68 | *** | *** | ** |
| | C31H35N9O3 | | | | |
| 1099 | 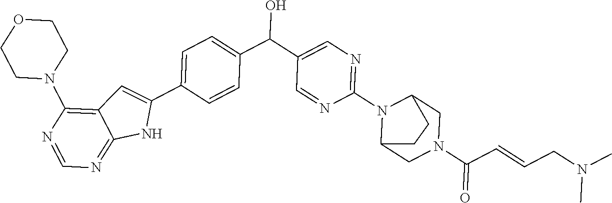 [SMILES: CN(C)C/C=C/C(=O)N1CC2CCC(C1)N2c1ncc(C(O)c2ccc(-c3cc4c(N5CCOCC5)ncnc4[nH]3)cc2)cn1] | 609.73 | *** | *** | * |
| | C33H39N9O3 | | | | |
| 1100 | 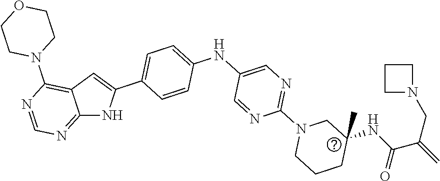 [SMILES: C=C(CN1CCC1)C(=O)N[C@]1(C)CCCN(c2ncc(Nc3ccc(-c4cc5c(N6CCOCC6)ncnc5[nH]4)cc3)cn2)C1] | 608.75 | *** | *** | * |
| | C33H40N10O2 | | | | |
| 1101 | 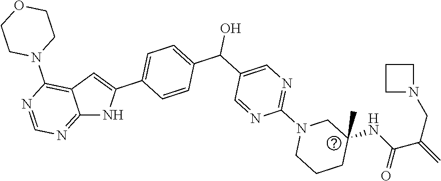 [SMILES: C=C(CN1CCC1)C(=O)N[C@]1(C)CCCN(c2ncc(C(O)c3ccc(-c4cc5c(N6CCOCC6)ncnc5[nH]4)cc3)cn2)C1] | 623.76 | *** | *** | * |
| | C34H41N9O3 | | | | |
| 1102 | 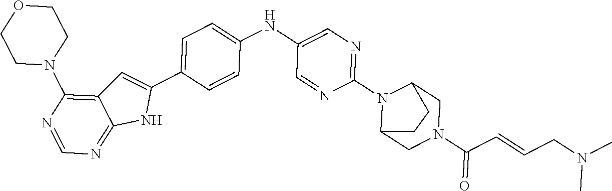 [SMILES: CN(C)C/C=C/C(=O)N1CC2CCC(C1)N2c1ncc(Nc2ccc(-c3cc4c(N5CCOCC5)ncnc4[nH]3)cc2)cn1] | 594.72 | **** | **** | * |
| | C32H38N10O2 | | | | |
| 1103 | 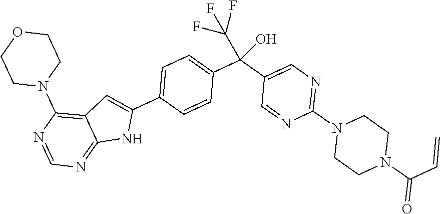 [SMILES: C=CC(=O)N1CCN(c2ncc(C(O)(c3ccc(-c4cc5c(N6CCOCC6)ncnc5[nH]4)cc3)C(F)(F)F)cn2)CC1] | 594.6 | *** | ** | ** |
| | C29H29F3N8O3 | | | | |

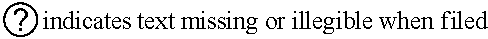

TABLE 2
Comparative Compounds

| | | | | MV- | | | |
|---|---|---|---|---|---|---|---|
| | | MOLM13- | | 4:11 | MV- | K-562- | |
| Com- | | DAY 4 | MOLM13- | DAY 4 | 4:11- | DAY 4 | K-562- |
| pound | | MEAN | DAY 4 | MEAN | DAY 4 | MEAN | DAY 4 |
| ID | | IC₅₀ | IC₅₀ | IC₅₀ | IC₅₀ | IC₅₀ | IC₅₀ |
| C-1 | 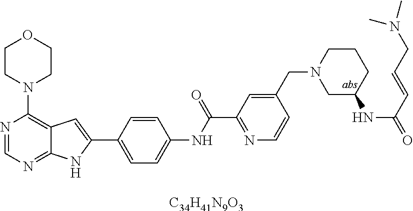 [SMILES: CN(C)C/C=C/C(=O)N[C@@H]1CCCN(Cc2ccnc(C(=O)Nc3ccc(-c4cc5c(N6CCOCC6)ncnc5[nH]4)cc3)c2)C1 ; "C34H41N9O3"] | 0.5152 | * | 0.581 | * | 3.1276 | * |
| C-2 | 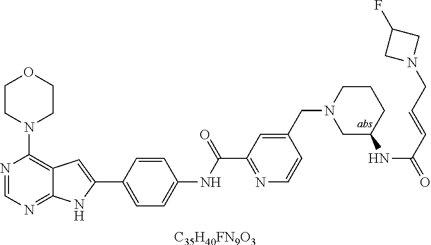 [SMILES: O=C(/C=C/CN1CC(F)C1)N[C@@H]1CCCN(Cc2ccnc(C(=O)Nc3ccc(-c4cc5c(N6CCOCC6)ncnc5[nH]4)cc3)c2)C1 ; "C35H40FN9O3"] | 0.6751 | * | 1.117 | * | 6.4878 | * |
| C-3 | 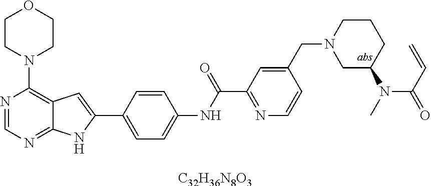 [SMILES: C=CC(=O)N(C)[C@@H]1CCCN(Cc2ccnc(C(=O)Nc3ccc(-c4cc5c(N6CCOCC6)ncnc5[nH]4)cc3)c2)C1 ; "C32H36N8O3"] | 0.4114 | ** | 0.4931 | ** | 1.9741 | * |
| C-4 | 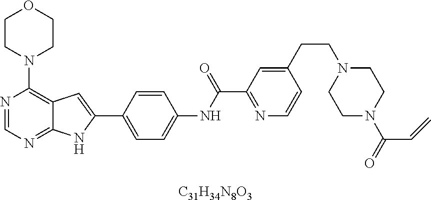 [SMILES: C=CC(=O)N1CCN(CCc2ccnc(C(=O)Nc3ccc(-c4cc5c(N6CCOCC6)ncnc5[nH]4)cc3)c2)CC1 ; "C31H34N8O3"] | 0.4838 | ** | 0.4528 | ** | 0.7704 | * |
| C-5 | 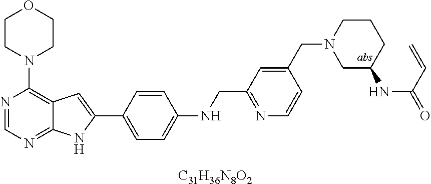 [SMILES: C=CC(=O)N[C@@H]1CCCN(Cc2ccnc(CNc3ccc(-c4cc5c(N6CCOCC6)ncnc5[nH]4)cc3)c2)C1 ; "C31H36N8O2"] | 1.0569 | * | 0.6709 | * | 10 | * |
| C-6 | 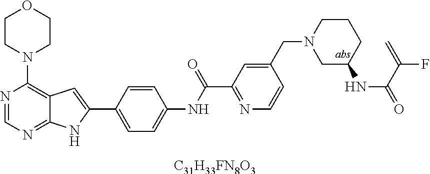 [SMILES: C=C(F)C(=O)N[C@@H]1CCCN(Cc2ccnc(C(=O)Nc3ccc(-c4cc5c(N6CCOCC6)ncnc5[nH]4)cc3)c2)C1 ; "C31H33FN8O3"] | 0.2614 | ** | 1.5471 | * | 10 | * |
| C-7 | 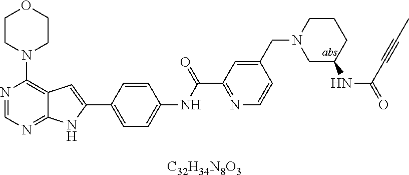 [SMILES: CC#CC(=O)N[C@@H]1CCCN(Cc2ccnc(C(=O)Nc3ccc(-c4cc5c(N6CCOCC6)ncnc5[nH]4)cc3)c2)C1 ; "C32H34N8O3"] | 0.2101 | ** | 0.2776 | ** | 0.9871 | * |
| C-8 | 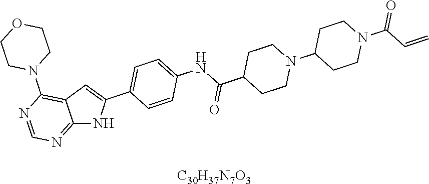 [SMILES: C=CC(=O)N1CCC(N2CCC(C(=O)Nc3ccc(-c4cc5c(N6CCOCC6)ncnc5[nH]4)cc3)CC2)CC1 ; "C30H37N7O3"] | | | 0.1825 | | 0.9911 | |

### Example B1C

The present example evaluates the ability of compounds to inhibit proliferation of solid tumor cells. The inhibitory effect on cell proliferation was investigated in lines comprising human pancreatic adenocarcinoma, colorectal carcinoma, and non-small cell lung cancer. Cells were maintained following supplier recommended conditions, in RPMI-1640 medium (ThermoFisher catalog no. 61870036) supplemented with 10% of heat inactivated FBS (ThermoFisher catalog no. A31605) and 1% Pen-Strep (ThermoFisher catalog no. 10378016) and cultured at 37° C. in a humidified incubator with 5% CO₂.

TABLE 3a

| Cell Line | Disease | Source | Catalog Number |
|---|---|---|---|
| A427 | NSCLC | ATCC | HTB-53 |
| A549 | NSCLC | ATCC | CCL-185 |
| ASPC-1 | PDAC | ATCC | CRL-1682 |
| COLO205 | CRC | ATCC | CCL-222 |
| COLO320 | CRC | ATCC | CCL-220 |
| HCT116 | CRC | ATCC | CCL-247 |
| LU99 | NSCLC | RIKEN | RCB1900 |
| MIA PACA 2 | PDAC | ATCC | CRL-1420 |
| NCI-H2122 | NSCLC | ATCC | CRL-5985 |
| NCI-H358 | NSCLC | ATCC | CRL-5807 |
| NCI-H460 | NSCLC | ATCC | HTB-177 |
| PANC1 | PDAC | ATCC | CRL-1469 |
| PK45H | PDAC | RIKEN | RCB1973 |
| Suit 2 | PDAC | DSMZ | JCRB1094 |
| SW48 | CRC | ATCC | CCL-231 |
| SW620 | CRC | ATCC | CCL-227 |

Cell viability is measured through the detection of metabolically active live cells. ATP is present in all metabolically active cells and is considered as a marker for viable cells and is determined with the CellTiter-Glo kit (Promega catalog no. G7572). The kit measures ATP based on the production of luminescence by the reaction of ATP with added UltraGlo recombinant luciferase (Kawano et al., 2016, PLOS One, 8; 11 (7): e0158888).

One day before the experiment, cells were detached with TrypLE (ThermoFisher catalog no. 12604054), quantified using the Vi-CELL XR Cell Viability (Beckman Coulter) analyzer and resuspended in fresh medium at a cell density of 5000 cells/mL, with the exception of HCT116 and SW620, which were at 2500 cells/mL. Cells were seeded at 200 μL/well in 96-well cell-culture plates and incubated overnight at 37° C. in a humidified incubator with 5% CO₂. Test compounds are dissolved in DMSO (Sigma catalog no. D8418; purity ≥99.9%) and prepared as 10 mM stock solutions. Eight concentrations of test compound in 3-fold serial dilutions were assessed in duplicate in an individual test occasion. Compounds were added at desired concentrations using a D300e compound dispenser (Tecan Trading AG) with shaking after each compound addition. The final DMSO concentration was maintained at 0.1% across all wells of the assay plate. Wells with untreated cells (0.1% DMSO) represent 100% proliferation. Assay plates were incubated at 37° C. in a humidified incubator with 5% CO₂.

On Day 4, viable cell counts were determined by quantifying ATP content using the CellTiter-Glo reagent from Promega. For this, assay plates were equilibrated to room temperature for approximately 15 min, and the CellTiter-Glo reagent was added at 40 μL/well. Contents were mixed for 30 minutes on an orbital shaker at 500 rpm to induce cell lysis. Luminescence was read using a Synergy Neo 2 (Agilent Technologies) plate reader.

Results were expressed as percentage of inhibition compared to the 0.1% DMSO negative control and was calculated as follows: % inhibition=100−[(RLU sample)×100/(RLU no-drug control)].

Dose response curves were plotted using GraphPad and IC₅₀ values were calculated by non-linear regression using 4 parameter-logistic equation.

### Summary of Findings:

As observed from the results, compounds exhibit potent anti-leukemic activity against solid tumors comprising human pancreatic adenocarcinoma, colorectal carcinoma, and non-small cell lung cancer.

TABLE 3b
Comparative data in various cell lines

| | IC₅₀ | | | IC₅₀ | | IC₅₀ | | | | | |
|---|---|---|---|---|---|---|---|---|---|---|---|
| | (uM) | IC₅₀ | IC₅₀ | (uM) | IC₅₀ | (uM) | IC₅₀ | IC₅₀ | IC₅₀ | IC₅₀ | IC₅₀ |
| | OCI- | (uM) | (uM) | MIA- | (uM) | NCI- | (uM) | (uM) | (uM) | (uM) | (uM) |
| | AML3- | HCT116- | COLO205- | PACA-2- | LU99- | H358- | COLO320- | ASPC-1- | A427- | PK45H- | SW48- |
| Compound | Day 4 | Day 4 | Day 4 | Day 4 | Day 4 | Day 4 | Day 4 | Day 4 | Day 4 | Day 4 | Day 4 |
| ID | MEAN | MEAN | MEAN | MEAN | MEAN | MEAN | MEAN | MEAN | MEAN | MEAN | MEAN |
| C-1 | 0.5537 | 0.691 | 0.7187 | 1.050 | 1.478 | 1.043 | 5.556 | 2.191 | 0.6675 | 1.397 | 0.477 |
| C-2 | | 2.883 | 2.970 | 4.413 | 5.904 | 4.973 | 10.000 | 3.901 | 1.162 | 4.783 | 2.064 |
| C-3 | | 2.561 | 2.793 | 4.707 | 4.407 | 4.101 | 6.150 | 5.492 | 3.823 | 5.430 | 1.972 |
| C-4 | | 0.1488 | 0.1604 | 0.633 | 0.4166 | 0.1665 | 0.368 | 0.2594 | 0.1213 | 0.7958 | 0.2163 |
| C-5 | | 3.202 | 3.255 | 4.508 | 4.964 | 4.344 | 8.216 | 6.653 | 3.118 | 5.336 | 4.178 |
| C-6 | | 0.3337 | 0.7521 | 5.319 | 4.113 | 3.919 | 0.9313 | 10.000 | 0.4257 | 5.042 | 0.8149 |
| C-7 | 0.372 | 0.2815 | 0.1923 | 0.4624 | 0.3925 | 0.3692 | 0.4275 | 0.4106 | 0.181 | 0.4964 | 0.2531 |
| C-8 | 0.2819 | 1.655 | | | | | 10.000 | | | | |
| 310 | | 1.321 | 1.403 | 1.502 | 4.755 | 2.500 | 10.000 | 10.000 | 1.222 | 2.752 | 1.159 |
| 314 | | 4.640 | 4.656 | 4.651 | 4.979 | 4.475 | 5.189 | 7.813 | 4.215 | 5.552 | 3.451 |
| 401 | | 0.7677 | 0.4858 | 0.9458 | 0.805 | 0.7087 | 0.6441 | 0.7986 | 0.3998 | 1.056 | 0.4223 |
| 402 | 2.107 | 0.3442 | 0.1732 | 0.4437 | 10.000 | 10.000 | 10.000 | 10.000 | 0.1295 | 3.911 | 0.1605 |
| 407 | 0.397 | 0.2898 | 0.3287 | 1.059 | 10.000 | 4.287 | 10.000 | 10.000 | 0.1374 | 10.000 | 0.1308 |
| 419 | | 1.417 | 1.229 | 1.365 | 3.291 | 2.069 | 10.000 | 7.601 | 0.6552 | 2.365 | 1.105 |
| 429 | 0.1527 | 0.1556 | 0.1541 | 0.2931 | 0.2293 | 0.2789 | 0.2253 | 0.4676 | 0.1717 | 0.3193 | 0.1228 |
| 451 | | 0.0499 | 0.0434 | 0.0502 | 0.0605 | 0.047 | 0.0475 | 0.0816 | 0.0479 | 0.0884 | 0.0472 |
| 601 | 0.0068 | 0.0076 | 0.0064 | 0.0114 | 0.0119 | 0.0113 | 0.0451 | 0.0249 | 0.0055 | 0.0127 | 0.0123 |
| 610 | 0.1576 | | | 0.1262 | | | | | | | |
| 616 | 0.3406 | | | 0.1597 | | | | | | | |
| 626 | 3.494 | 4.437 | 2.824 | 4.797 | 4.163 | 3.800 | 5.934 | 6.542 | 2.845 | 4.609 | 2.711 |
| 627 | 2.901 | 1.830 | | | | 2.805 | | | 1.574 | | 1.975 |
| 639 | 0.0074 | 0.0053 | 0.0075 | | | 0.0102 | 0.0407 | 0.0161 | 0.0045 | 0.0169 | |
| 703 | 0.1143 | | | 0.0971 | | | | | | | |
| 705 | 0.0704 | 0.0759 | 0.0576 | 0.0648 | 0.1177 | 0.1034 | 0.3519 | 0.5215 | 0.05 | 0.0917 | 0.0521 |
| 906 | 0.4431 | 0.2511 | 0.4274 | 0.46 | 0.5183 | 0.4636 | 1.226 | 0.732 | 0.2331 | 0.6983 | 0.4411 |
| 915 | 0.0674 | 0.0568 | 0.0912 | 0.0876 | 0.2707 | 0.1474 | 3.578 | 0.9575 | 0.0676 | 0.3006 | 0.0611 |
| 921 | 0.9231 | | | 1.912 | | | | | | | |
| 929 | 0.3705 | | | 0.7447 | | | | | | | |
| 956 | 0.5215 | 1.034 | | 1.997 | | 1.784 | 10.000 | 3.456 | 0.4177 | | |
| 958 | 0.4557 | | | 0.4889 | | | | | | | |
| 960 | 0.4029 | | | 0.5131 | | | | | | | |
| 970 | 0.1805 | 0.2089 | | 0.3524 | | 0.4067 | 5.248 | 2.524 | 0.1511 | | |
| 971 | 0.0431 | 0.0061 | 0.0109 | 0.0565 | 0.0296 | 0.0253 | 0.0532 | 0.1132 | 0.0145 | 0.0955 | 0.0144 |
| 972 | 0.3916 | 0.8515 | | 0.9864 | | 0.8021 | 5.237 | 4.342 | 0.6329 | | |
| 1002 | 0.0227 | 0.0148 | | 0.0292 | | 0.0426 | 0.2082 | 0.2735 | 0.0203 | | |
| 1004 | 1.032 | 1.228 | | 0.8511 | | 1.198 | 10.000 | 5.988 | 0.8963 | | |
| 1011 | 10.000 | 10.323 | | 0.41 | | 16.576 | 10.000 | 10.000 | 1.662 | | |
| 1012 | 0.6595 | 0.8915 | | 1.226 | | 1.306 | 5.217 | 4.449 | 0.7585 | | |
| 1033 | 0.0467 | 0.027 | 0.0266 | 0.0467 | 0.0514 | 0.0575 | 0.6868 | 0.1378 | 0.0376 | 0.0632 | 0.0422 |
| 1036 | 0.0587 | 0.0154 | 0.0346 | 0.0662 | 0.0647 | 0.0491 | 0.0767 | 0.186 | 0.0327 | 0.107 | 0.0341 |

TABLE 3c
Comparative data in various cell lines

| | | | | | | | | IC₅₀ | | | |
|---|---|---|---|---|---|---|---|---|---|---|---|
| | IC₅₀ | IC₅₀ | IC₅₀ | IC₅₀ | IC₅₀ | IC₅₀ | IC₅₀ | (uM) | IC₅₀ | IC₅₀ | IC₅₀ |
| | (uM) | (uM) | (uM) | (uM) | (uM) | (uM) | (uM) | NCI- | (uM) | (uM) | (uM) |
| | Suit-2- | SW620- | PANC1- | JJN3- | MM.1S- | KMS-20- | OPM2- | H2122- | KMS-12-BM- | SKMM2- | SKMM1- |
| Compound | Day 4 | Day 4 | Day 4 | Day 4 | Day 4 | Day 4 | Day 4 | Day 4 | Day 4 | Day 4 | Day 4 |
| ID | MEAN | MEAN | MEAN | MEAN | MEAN | MEAN | MEAN | MEAN | MEAN | MEAN | MEAN |
| C-1 | 3.6021 | 1.3217 | | | | | | | | | |
| C-2 | 7.6233 | 5.0732 | | | | | | | | | |
| C-3 | 7.8792 | 4.1857 | | | | | | | | | |
| C-4 | 1.1261 | 0.7464 | | | | | | | | | |
| C-5 | 10.087 | 3.9661 | | | | | | | | | |
| C-6 | 10 | 6.8764 | | | | | | | | | |
| C-7 | 0.6561 | 0.4006 | | | | | | | | | |
| C-8 | | 6.7246 | | | | | | | | | |
| 310 | 10.43 | 2.2993 | | | | | | | | | |
| 314 | 10.782 | 7.2779 | | | | | | | | | |
| 401 | 1.7751 | 0.9818 | | | | | | | | | |
| 402 | 10 | 10 | | | | | | | | | |
| 407 | 10 | 10 | | | | | | | | | |
| 419 | 8.7875 | 2.8702 | | | | | | | | | |
| 429 | 0.5338 | 0.3561 | | | | | | | | | |
| 451 | 0.1219 | 0.0597 | | | | | | | | | |
| 601 | 0.0543 | 0.0046 | | | | | | | | | |
| 610 | | | 1.1763 | | | | | | | | |
| 616 | | | 0.3372 | | | | | | | | |
| 626 | 7.8205 | 5.2072 | 4.3243 | 1.704 | 2.5564 | 2.1004 | 2.901 | 1.963 | 3.2667 | 3.4494 | 3.3466 |
| 627 | 5.455 | 5.3521 | | | | | | | | | |
| 639 | | 0.0089 | | 0.0036 | 0.0035 | 0.0105 | 0.0071 | 0.0108 | 0.0043 | 0.0302 | 0.0044 |
| 703 | | | 0.6526 | | | | | | | | |
| 705 | 0.295 | 0.0548 | | | | | | | | | |
| 906 | 1.3552 | 0.685 | 0.7367 | 0.2225 | 0.2307 | 0.4437 | 0.4023 | 0.3191 | 0.3732 | 0.7277 | 0.2708 |
| 915 | 1.3912 | 0.1824 | | 0.0523 | 0.0767 | 0.3438 | 0.1648 | 0.3633 | 0.0957 | 3.6629 | 0.1233 |
| 921 | | | 3.4834 | | | | | | | | |
| 929 | | | 1.1006 | | | | | | | | |
| 956 | | | 1.403 | | | | | | | | |
| 958 | | | 1.5758 | | | | | | | | |
| 960 | | | 6.3405 | | | | | | | | |
| 971 | 0.173 | 0.0128 | | | | | | | | | |
| 1033 | 0.4384 | 1.0744 | | | | | | | | | |
| 1036 | 0.215 | 0.0282 | | | | | | | | | |

TABLE 3d
Comparative data in various cell lines

| | | | IC₅₀ | | IC₅₀ | | | | | | |
|---|---|---|---|---|---|---|---|---|---|---|---|
| | IC₅₀ | IC₅₀ | (uM) | IC₅₀ | (uM) | IC₅₀ | IC₅₀ | IC₅₀ | IC₅₀ | IC₅₀ | IC₅₀ |
| | (uM) | (uM) | OCI- | (uM) | NCI- | (uM) | (uM) | (uM) | (uM) | (uM) | (uM) |
| | A549- | DB- | AML2- | KOPN-8- | H460- | OCI-Ly7- | OCI-Ly3- | DOHH2- | Pfeiffer- | U2932- | RS: 411- |
| Compound | Day 4 | Day 4 | Day 4 | Day 4 | Day 4 | Day 4 | Day 4 | Day 4 | Day 4 | Day 4 | Day 4 |
| ID | MEAN | MEAN | MEAN | MEAN | MEAN | MEAN | MEAN | MEAN | MEAN | MEAN | MEAN |
| 626 | 4.3324 | 5.6162 | 3.8046 | 4.4808 | 4.8806 | 5.9292 | 7.4607 | 3.2102 | 3.5573 | 4.3354 | 3.4131 |
| 627 | 4.3903 | 4.1847 | 3.2641 | 4.3384 | 4.362 | 3.9013 | 4.4788 | 2.2961 | 3.4324 | 3.5286 | 2.39 |
| 906 | 0.5665 | 0.6068 | 0.5039 | 0.4886 | 0.5398 | 0.5989 | 0.7938 | 0.2776 | 0.4473 | 0.508 | 0.3647 |

TABLE 3d
Comparative data in various cell lines

| | | IC₅₀ | | | | | | | | | IC₅₀ |
|---|---|---|---|---|---|---|---|---|---|---|---|
| | IC₅₀ | (uM) | IC₅₀ | IC₅₀ | IC₅₀ | IC₅₀ | IC₅₀ | IC₅₀ | IC₅₀ | IC₅₀ | (uM) |
| | (uM) | SU- | (uM) | (uM) | (uM) | (uM) | (uM) | (uM) | (uM) | (uM) | NCI- |
| | VAL- | DHL-8- | TOLEDO- | PBMC- | THP-1- | U937- | MV-4:11- | K-562- | AGS- | LS513- | H1792- |
| Compound | Day 4 | Day 4 | Day 4 | Day 5 | Day 4 | Day 4 | Day 7 | Day 7 | Day 4 | Day 4 | Day 4 |
| ID | MEAN | MEAN | MEAN | MEAN | MEAN | MEAN | MEAN | MEAN | MEAN | MEAN | MEAN |
| 626 | 3.3779 | 4.3862 | 4.0748 | 2.2415 | 6.0925 | 4.8712 | | | | | |
| 627 | 2.5106 | 3.2187 | 3.2777 | 1.9286 | 3.9846 | 3.4019 | | | | | |
| 906 | 0.4368 | 0.4906 | 0.4715 | 0.1991 | 0.5018 | 0.5094 | | | | | |

### Example B2: Pharmaceutical Compositions

The compositions described below are presented with a compound of Formula (L-I), (L-II), and (I) for illustrative purposes.

### Example B2a: Parenteral Composition

To prepare a parenteral pharmaceutical composition suitable for administration by injection, 100 mg of a water-soluble salt of a compound of Formula (L-I), (L-II), and (I) is dissolved in DMSO and then mixed with 10 mL of 0.9% sterile saline. The mixture is incorporated into a dosage unit form suitable for administration by injection.

### Example B2b: Oral Composition

To prepare a pharmaceutical composition for oral delivery, 100 mg of a compound of Formula (L-I), (L-II), and (I) is mixed with 750 mg of starch. The mixture is incorporated into an oral dosage unit for, such as a hard gelatin capsule, which is suitable for oral administration.

### Example B2c: Sublingual (Hard Lozenge) Composition

To prepare a pharmaceutical composition for buccal delivery, such as a hard lozenge, mix 100 mg of a compound of Formula (L-I), (L-II), and (I) with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example B2d: Inhalation Composition

To prepare a pharmaceutical composition for inhalation delivery, 20 mg of a compound of Formula (L-I), (L-II), and (I) is mixed with 50 mg of anhydrous citric acid and 100 mL of 0.9% sodium chloride solution. The mixture is incorporated into an inhalation delivery unit, such as a nebulizer, which is suitable for inhalation administration.

### Example B2e: Rectal Gel Composition

To prepare a pharmaceutical composition for rectal delivery, 100 mg of a compound of Formula (L-I), (L-II), and (I) is mixed with 2.5 g of methylcelluose (1500 mPa), 100 mg of methylparaben, 5 g of glycerin and 100 mL of purified water. The resulting gel mixture is then incorporated into rectal delivery units, such as syringes, which are suitable for rectal administration.

### Example B2f: Topical Gel Composition

To prepare a pharmaceutical topical gel composition, 100 mg of a compound of Formula (L-I), (L-II), and (I) is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topical administration.

### Example B2 g: Ophthalmic Solution Composition

To prepare a pharmaceutical ophthalmic solution composition, 100 mg of a compound of Formula (L-I), (L-II), and (I) is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

At least some of the chemical names of compounds provided herein as given and set forth in this application, may have been generated on an automated basis by use of a commercially available chemical naming software program, and have not been independently verified In the instance where the indicated chemical name and the depicted structure differ, the depicted structure will control. In the chemical structures where a chiral center exists in a structure but no specific stereochemistry is shown for the chiral center, both enantiomers associated with the chiral structure are encompassed by the structure.

What is claimed is:

1. A compound according to formula (L-I): 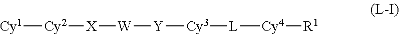
or a stereoisomer thereof; or a pharmaceutically acceptable salt thereof,
wherein:
Cy¹ is substituted or unsubstituted 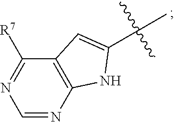
and the substitution on Cy¹ is C₁-C₄ alkyl, CN, or halo;
each of Cy² and Cy³ is independently substituted or unsubstituted cycloalkylene, substituted or unsubstituted heterocycloalkylene, substituted or unsubstituted arylene, or substituted or unsubstituted heteroarylene;
Cy⁴ is absent, substituted or unsubstituted cycloalkylene, substituted or unsubstituted heterocycloalkylene, substituted or unsubstituted arylene, or substituted or unsubstituted heteroarylene;
L is a single bond, or substituted or unsubstituted —CH₂—; and the substitution on CH₂ is Me, Et, OH, OMe, OEt, or O-i-Pr.
R⁷ is a 4-10 membered substituted or unsubstituted heterocycloalkyl ring having 1-2 heteroatoms independently selected from nitrogen, oxygen, and sulfur; substituted or unsubstituted phenyl; an 8-10 membered substituted or unsubstituted bicyclic aryl ring; or a 5-6 membered substituted or unsubstituted heteroaryl ring having 1-4 heteroatoms independently selected from nitrogen, oxygen, and sulfur; and the substitution on the phenyl, and the bicyclic aryl is 1, 2, or 3 groups independently selected from C₁-C₄ alkyl, hydroxy, CN, C₁-C₄ alkoxy;
i) X-W-Y is —NH—, —O—, —NH—C(R^{3a})(R^{3b})-, —S(O)₂- or —NH—C(O)—; and R¹ is —B—C(R^{6a})═C(R^{6b})-C(O)-R^{6c},-B-C(R^{6a})═C(R^{6b})-S(O)-R^{6c},-B-C(R^{6a})═C(R^{6b})-S(O)₂-R^{6c},-B-C(R^{6a})-C(R^{6b})-P (O)-R^{6a}R^{6b}; or —B—C(R^{6a})-C(R^{6b})-P(O)-OR^{6a}OR^{6b}; B is substituted or unsubstituted C₁₋₄ alkylene; R^{6c} is substituted or unsubstituted alkoxy, substituted or unsubstituted amino, or substituted or unsubstituted heterocycloalkyl, having 1-2 heteroatoms independently selected from nitrogen, oxygen, and sulfur; each R^{6a} and R^{6b} is independently H, CN, halo, or substituted or unsubstituted C₁₋₆ alkyl; and the substitution on C1-6 alkyl is 1, 2, or 3 groups independently selected from halo, alkoxy, alkylamino, dialkylamino, and heterocycloalkyl;
ii) X-W-Y is —NH—, —O—, —NH—C(R^{3a})(R^{3b})-, or —S(O)₂—; and R¹ is —C(O)—C(R^{6a})═C(R^{6b})R⁶,-S(O)-C(R^{6a})═C(R^{6b})R^{6c},-S(O)₂-C(R^{6a})-C(R^{6b})R^{6c},-NR^{3c}-C(O)—C(R^{6a})-C(R^{6b})R^{6c},-NR^{3c}—S(O)-C(R^{6a})═C(R^{6b})R^{6c}, or —NR^{3c}—S(O)₂-C(R^{6a})-C(R^{6b})Roc, and each R^{6a}, R^{6b}, and R^{6c} is independently H, CN, halo, or substituted or unsubstituted C₁₋₆ alkyl; or R^{6a} and R^{6b} are joined together to form a bond, and R^{6c} is independently H, CN, halo, or substituted or unsubstituted C₁₋₆ alkyl; and wherein at least one of R^{3a} and R^{3b} is substituted alkyl;
iii) X-W-Y is —NH—C(O)—; and R¹ is —C(O)—C(R^{6a})═C(R^{6b})R^{6c},-S(O)-C(R^{6a})═C(R^{6b})R^{6c},-S(O)₂-C(R^{6a})═C(R^{6b}) R^{6c},-NR^{3c}-C(O)₂-C(R^{6a})═C(R^{6b})R^{6c},-NR^{3c}—S(O)-C(R^{6a})═C(R^{6b})R^{6c}, or -NR^{3c}—S(O)₂-C(R^{6a})-C(R^{6b})R^{6c}; and R^{6a} is alkyl substituted with alkoxy, alkylamino, dialkylamino, or substituted or unsubstituted heterocycloalkyl, and each R^{6b}, and R^{6c} is independently H, CN, halo, or substituted or unsubstituted C₁₋₆ alkyl; or
iv) X-W-Y is —NH—C(O)—; and R¹ is —C(O)—C(R^{6a})═C(R^{6b})R^{6c},-S(O)-C(R^{6a})═C(R^{6b})R^{6c},-S(O)₂-C(R^{6a})═C(R^{6b}) R^{6c},-NR^{3c}-C(O)—C(R^{6a})═C(R^{6b})R^{6c},-NR^{3c}—S(O)-C(R^{6a})═C(R^{6b})R^{6c}, or -NR^{3c}—S(O)₂-C(R^{6a})═C(R^{6b})R^{6c}; each R^{6a}, R^{6b}, and R^{6c} is independently H, CN, halo, or substituted or unsubstituted C₁₋₆ alkyl; or R^{6a} and R^{6b} are joined together to form a bond, and Roc is independently H, CN, halo, or substituted or unsubstituted C₁₋₆ alkyl; and Cy⁴ is substituted or unsubstituted heterocycloalkyl, and the heterocycloalkyl is a bridged heterocycloalkyl;
R^{3a} is H or substituted or unsubstituted C₁₋₄ alkyl; and each of R^{3b}, and R^{3c} is independently H, OH, substituted or unsubstituted C₁₋₄ alkyl, substituted or unsubstituted C₁₋₄ alkoxy;
and wherein
the substitution on each alkyl is 1, 2, or 3 groups independently selected from halo, CN, hydroxy, and C₁-C₄ alkoxy;
the substitution on alkylene is 1, 2, or 3 groups independently selected from C₁-C₄ alkyl, CN, hydroxy, and halo;
the substitution on —N(H)— is C₁-C₄ alkyl;
the substitution on each alkoxy is 1, 2, or 3 groups independently selected from halo, CN, and substituted or unsubstituted C₁-C₄ alkyl;
the substitution on each cycloalkyl is 1, 2, or 3 groups independently selected from halo, CN, hydroxy, substituted or unsubstituted C₁-C₄ alkyl, and substituted or unsubstituted C₁-C₄ alkoxy;
the substitution on each heterocycloalkyl is 1, 2, or 3 groups independently selected from halo, CN, hydroxy, substituted or unsubstituted C₁-C₄ alkyl, and substituted or unsubstituted C₁-C₄ alkoxy;
the substitution on each aryl is 1, 2, or 3 groups independently selected from halo, haloalkyl, amino, dialkylamino, amido, CN, hydroxy, substituted or unsubstituted C₁-C₄ alkyl, and substituted or unsubstituted C₁-C₄ alkoxy;
the substitution on each heteroaryl is 1-3 groups independently selected from halo, haloalkyl, amino, dialkylamino, amido, CN, hydroxy, substituted or unsubstituted C₁-C₄ alkyl, substituted or unsubstituted C₁-C₄ alkoxy, and oxo;
or the compound is any one of the compounds listed in Table 1G, 1H, 1J, and 1K;
provided that the compound is other than:

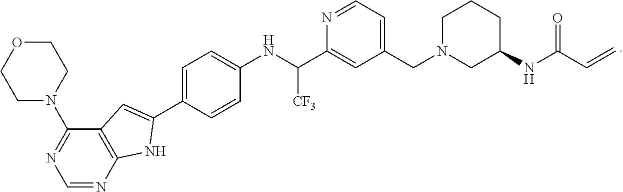 [SMILES: C=CC(=O)N[C@@H]1CCCN(Cc2ccnc(C(Nc3ccc(-c4cc5c(N6CCOCC6)ncnc5[nH]4)cc3)C(F)(F)F)c2)C1]

2. The compound according to claim 1, wherein X-W-Y is —NH—, —O—, —NH—C(R^{3a})(R^{3b})-, or —S(O)₂—; and R¹ is-C(O)—C(R^{6a})═C(R^{6b})R^{6c},-S(O)-C(R^{6a})═C(R^{6b})R^{6c},-S(O)₂-C(R^{6a})═C(R^{6b})R^{6c},-NR^{3c}-C(O)—C(R^{6a})═C(R^{6b}) R^{6c},-NR^{3c}—S(O)-C(R^{6a})-C(R^{6b})R^{6c}, or -NR^{3c}—S(O)₂-C(R^{6a})-C(R^{6b})R^{6c}; and each R^{6a}, R^{6b}, and R^{6c} is independently H, CN, halo, or substituted or unsubstituted C₁₋₆ alkyl; or R^{6a} and R^{6b} are joined together to form a bond, and R^{6c} is independently H, CN, halo, or substituted or unsubstituted C₁₋₆ alkyl; and wherein at least one of R^{3a} and R^{3b} is substituted alkyl.

3. The compound according to claim 1, X-W-Y is —NH—C(O)—; and R¹ is —C(O)—C(R^{6a})-C(R^{6b})R⁶,-S(O)-C(R^{6a})═C(R^{6b})R^{6c},-S(O)₂-C(R^{6a})═C(R^{6b})R^{6c},-NR^{3c}-C(O)—C(R^{6a})═C(R^{6b})R^{6c},-NR^{3c}—S(O)-C(R^{6a})═C(R^{6b})R^{6c}, or —NR^{3c}—S(O)₂-C(R^{6a})-C(R^{6b})R^{6c}, and R^{6a} is alkyl, substituted with alkoxy, alkylamino, dialkylamino, or substituted or unsubstituted heterocycloalkyl, and each R^{6b}, and R^{6c} is independently H, CN, halo, or substituted or unsubstituted C₁₋₆ alkyl.

4. The compound according to claim 1, X-W-Y is —NH—C(O)—; and R¹ is —C(O)—C(R^{6a})-C(R^{6b})R^{6c},-S(O)-C(R^{6a})═C(R^{6b})R^{6c},-S(O)₂-C(R^{6a})═C(R^{6b})R^{6c},-NR^{3c}-C(O)—C(R^{6a})═C(R^{6b})R^{6c},-NR^{3c}—S(O)-C(R^{6a})═C(R^{6b})R^{6c}, or —NR^{3c}—S(O)₂-C(R^{6a})═C(R^{6b})R^{6c}; each R^{6a}, R^{6b}, and R^{6c} is independently H, CN, halo, or substituted or unsubstituted C₁₋₆ alkyl; or R^{6a} and R^{6b} are joined together to form a bond, and R^{6c} is independently H, CN, halo, or substituted or unsubstituted C₁₋₆ alkyl; and Cy⁴ is substituted or unsubstituted heterocycloalkylene, and the heterocycloalkylene is a bridged heterocycloalkylene.

5. The compound according to any one of claims 1-4, wherein-X—W-Y— is —NH—, —O—, —NH—C(R^{3a})(R^{3b})-, or —S(O)₂-.

6. The compound according to any one of claims 1-4, wherein-X—W-Y— is —NH—C(O)—.

7. The compound according to any one of claims 1-4, wherein-X—W-Y— is -N(H)- or —O—.

8. The compound according to any one of claims 1-4, wherein-X—W-Y— is —NH—C(R^{3a})(R^{3b})-; and one of R^{3a} and R^{3b} is CF₃.

9. The compound according to any one of claims 1-4, wherein-X—W-Y— is -N(H)-C(H)(CF₃)—.

10. The compound according to any one of claims 1-4, wherein the compound is according to formula LXIa, LXIb, LXII, LXIII, LXIV, LXV, or LXVI, or a stereoisomer thereof; or a pharmaceutically acceptable salt thereof:

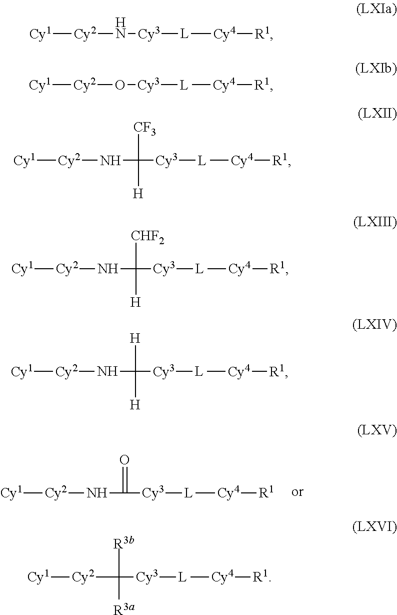

11. The compound according to any one of claims 1-10, wherein Cy² is substituted or unsubstituted phenylene, or substituted or unsubstituted pyridinylene; and each substitution is independently selected from 1, 2, or 3 of C₁-C₄ alkyl, hydroxy, CN, C₁-C₄ alkoxy, and halo.

12. The compound according to any one of claims 1-10, wherein Cy² is unsubstituted phenylene.

13. The compound according to any one of claims 1-12, wherein Cy³ is substituted or unsubstituted phenylene, pyridylene, pyrimidinylene, pyrrolidinylene, piperidinylene, or piperazinylene; and each substitution is independently selected from 1, 2, or 3 of C₁-C₄ alkyl, amino, alkylamino, dialkylamino, hydroxy, C₁-C₄ hydroxyalkyl, CN, C₁-C₄ alkoxy, and halo.

14. The compound according to any one of claims 1-13, wherein L is a single bond.

15. The compound according to any one of claims 1-13, wherein L is -CH₂—, or -C(Me)H-.

16. The compound according to any one of claims 1-15, wherein Cy⁴ is absent.

17. The compound according to any one of claims 1-15, wherein Cy⁴ is substituted or unsubstituted heterocycloalkylene; and each substitution is independently selected from 1, 2, or 3 of C₁-C₄ alkyl, amino, alkylamino, dialkylamino, hydroxy, C₁-C₄ hydroxyalkyl, CN, C₁-C₄ alkoxy, and halo.

18. The compound according to any one of claims 1-15, wherein Cy⁴ is substituted or unsubstituted azetidinylene, substituted or unsubstituted pyrrolidinylene, substituted or unsubstituted piperidinylene, substituted or unsubstituted piperazinylene, substituted or unsubstituted diazabicycloheptanylene, or substituted or unsubstituted diazabicyclooctanylene; and each substitution is independently selected from 1, 2, or 3 of C₁-C₄ alkyl, amino, alkylamino, dialkylamino, hydroxy, C₁-C₄ hydroxyalkyl, CN, C₁-C₄ alkoxy, and halo.

19. The compound according to any one of claims 1-15, wherein Cy⁴ is unsubstituted azetidinylene, unsubstituted pyrrolidinylene, unsubstituted piperidinylene, or unsubstituted piperazinylene.

20. The compound according to any one of claims 1-15, wherein Cy⁴ is azetidinylene, pyrrolidinylene, piperidinylene, or piperazinylene, each substituted with one or more alkyl, halo, or alkoxy.

21. The compound according to any one of claims 1-15, wherein Cy⁴ is azetidinylene, pyrrolidinylene, piperidinylene, or piperazinylene, each substituted with one or more Me, Et, F, (F)₂, or Cl.

22. The compound according to any one of claims 1-15, wherein Cy⁴ is diazabicycloheptanylene or diazabicyclooctanylene, each unsubstituted or substituted with one or more alkyl, halo, or alkoxy.

23. The compound according to any one of claims 1-15, wherein Cy⁴ is 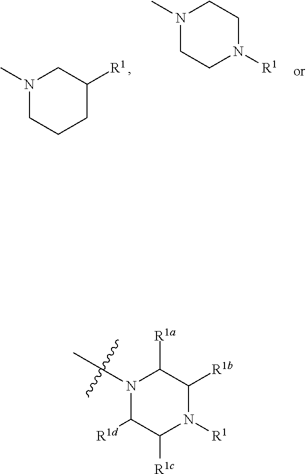
and wherein at least one pair of R^{1a} and R^{1c}, R^{1a} and R^{1d}, R^{1b} and R^{1c}, or R^{1b} and R^{1d} is joined together to form heterocycloalkyl, and the rest of R^{1a}, R^{1b}, R^{1c}, and R^{1d} are each H.

24. The compound according to any one of claims 1-15, wherein Cy⁴ is

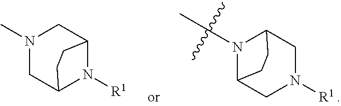

25. The compound according to any one of claims 1-24, wherein R⁷ is morpholinyl, piperidinyl, thiomorpholinyl; each unsubstituted or substituted with one or more Me, Et, F, (F)₂, (O)₂, or Cl.

26. The compound according to any one of claims 1-24, wherein R⁷ is

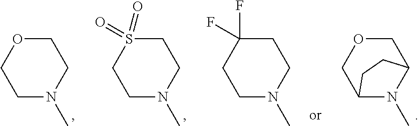

27. The compound according to claim 1, wherein the compound is according to formula (LXVIIa) or LXVIIb) 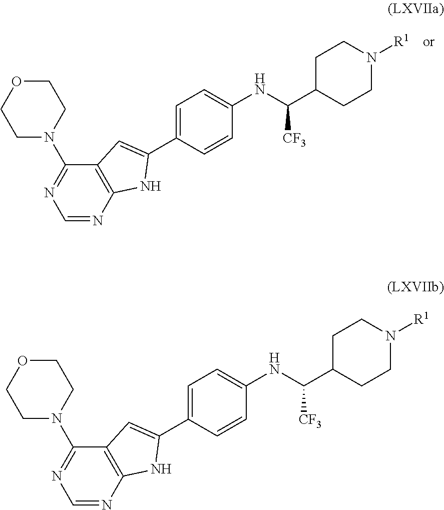
or a stereoisomer thereof; or a pharmaceutically acceptable salt thereof.

28. The compound according to claim 1, wherein the compound is according to formula (LXVIII) 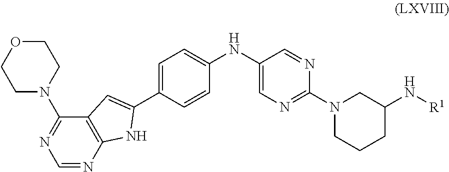
or a stereoisomer thereof; or a pharmaceutically acceptable salt thereof.

29. The compound according to claim 1, wherein the compound is according to formula (LXIXa), (LXIXb), or (LXIXc) 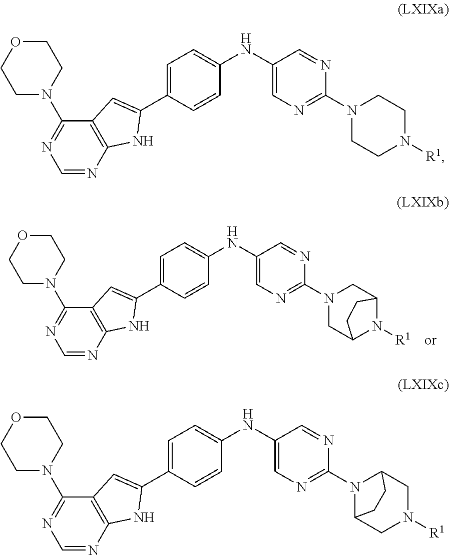
or a stereoisomer thereof; or a pharmaceutically acceptable salt thereof.

30. The compound according to claim 1, wherein the compound is according to formula (LXX) 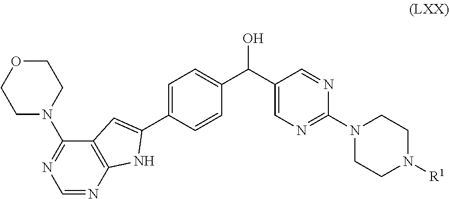
or a stereoisomer thereof; or a pharmaceutically acceptable salt thereof.

31. The compound according to claim 1, wherein the compound is according to formula (LXXIa) or (LXXIb) 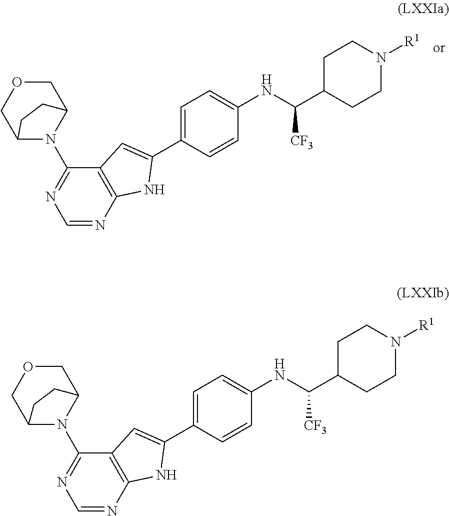
or a stereoisomer thereof; or a pharmaceutically acceptable salt thereof.

32. The compound according to claim 1, wherein the compound is according to formula (LXXII) 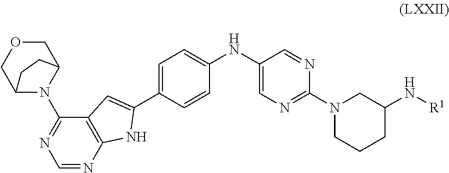
or a stereoisomer thereof; or a pharmaceutically acceptable salt thereof.

33. The compound according to claim 1, wherein the compound is according to formula (LXXIIIa), (LXXIIIb), or (LXXIIIc) 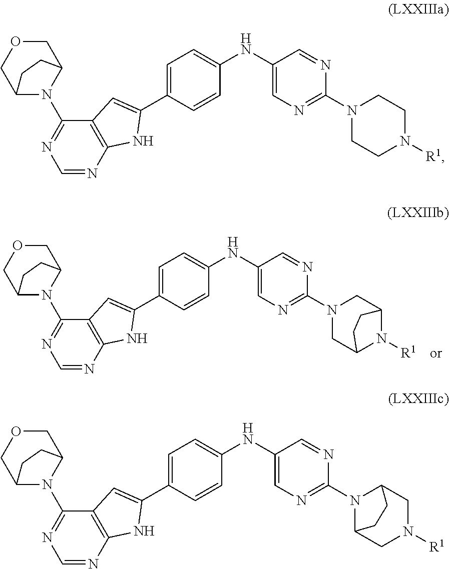
or a stereoisomer thereof; or a pharmaceutically acceptable salt thereof.

34. The compound according to claim 1, wherein the compound is according to formula (LXXIV) 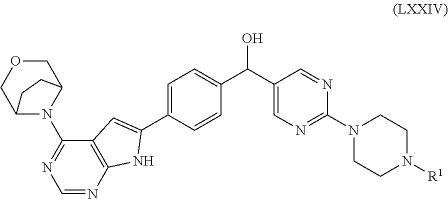
or a stereoisomer thereof; or a pharmaceutically acceptable salt thereof.

35. The compound according to claim 1, wherein the compound is according to formula (LXXVa) or (LXXVb) 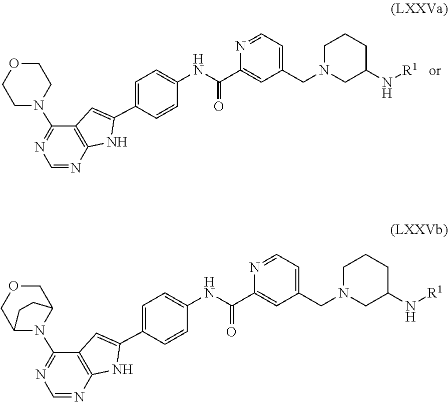
or a stereoisomer thereof; or a pharmaceutically acceptable salt thereof.

36. The compound according to claim 1, wherein the compound is according to formula (LXXVIa), (LXXVIb), (LXXVIc), (LXXVId), (LXXVIe), or (LXXVIf). 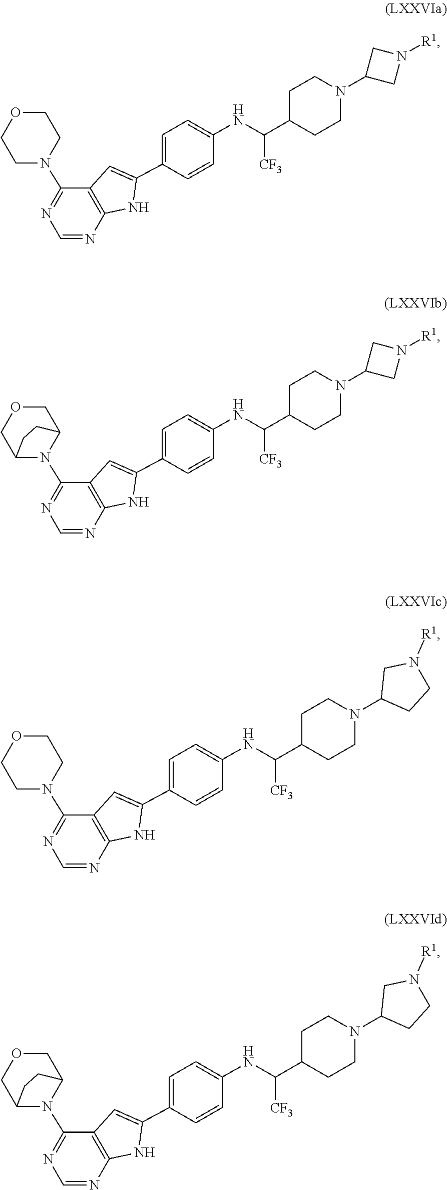 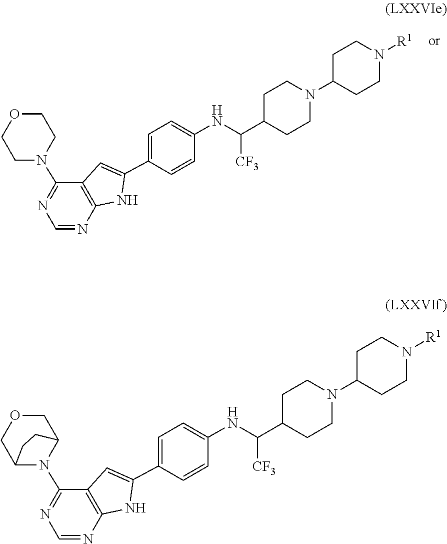
or a stereoisomer thereof; or a pharmaceutically acceptable salt thereof.

37. The compound according to claim 1, wherein the compound is according to formula (LXXVIIa), (LXXVIIb), (LXXVIIc), (LXXVIId), (LXXVIIe), or (LXXVIIf) 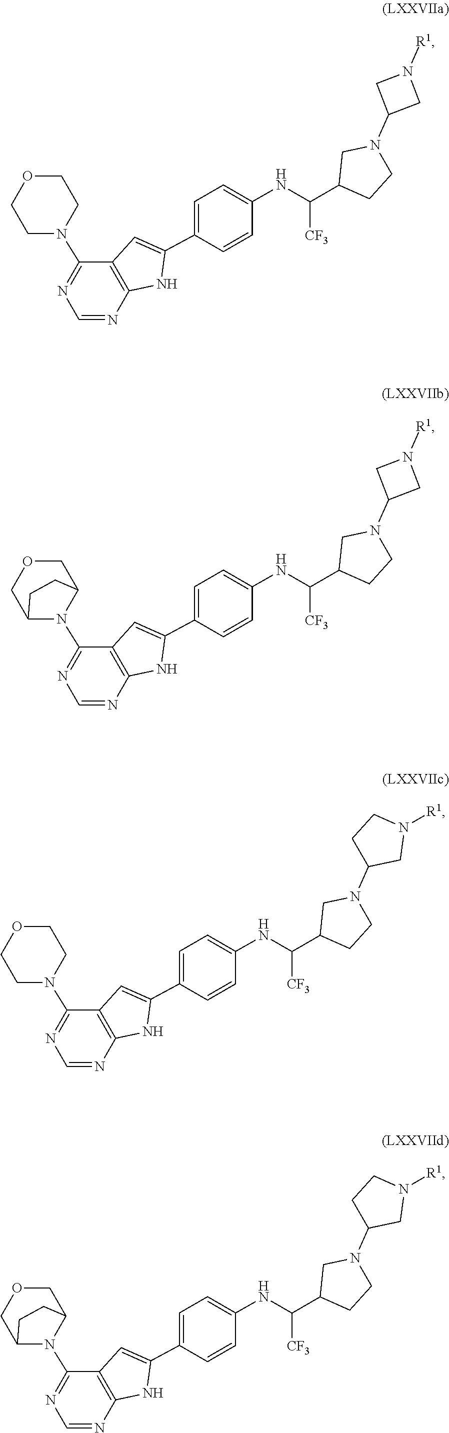 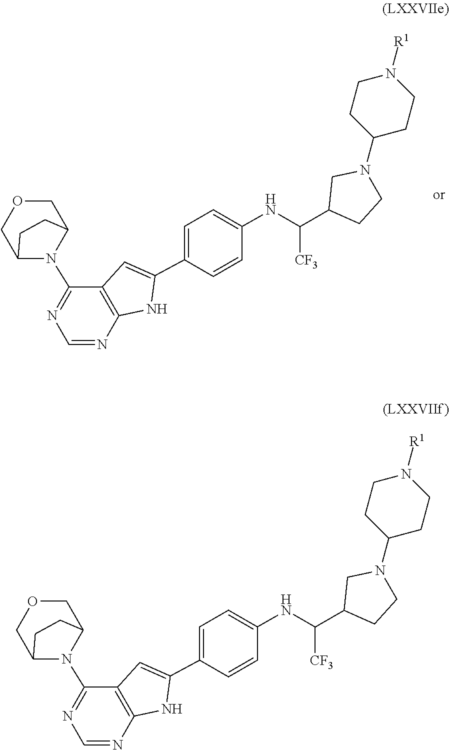
or a stereoisomer thereof; or a pharmaceutically acceptable salt thereof.

38. The compound according to claim 1, wherein the compound is according to formula (LXXVIII) 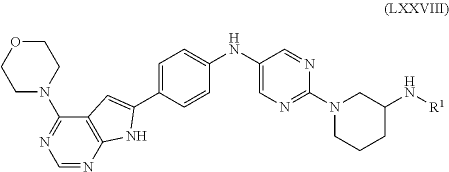
or a stereoisomer thereof; or a pharmaceutically acceptable salt thereof.

39. The compound according to any one of claims 1-38, wherein R¹ is —S(O)₂—CH═CH₂, —NH—C(O)—C≡CH,-C(O)-CH═CH₂,-C(O)—C≡CH, —NH—C(O)—CH═CH₂,-C(O)—C≡C-Me, or —NH—S(O)₂—CH═CH₂.

40. The compound according to any one of claims 1-38, wherein R¹ is —C(O)—CH═CH—CH₂OH, or -C(O)-CH═CH-OH.

41. The compound according to any one of claims 1-38, wherein R¹ is —NH—C(O)—C(CI)=CH₂, —NH—C(O)-C(F)=CH₂, —NH—C(O)—C(CH₂-NMe₂)=CH₂, —NH—S(O)₂-C(Me)=CH₂, —NH—C(O)—C(CH₂-morpholin-1-yl)=CH₂, or —NH—C(O)—C(CH₂-OMe)=CH₂.

42. The compound according to any one of claims 1-38, wherein R¹ is —NH—C(O)—C(CH₂-morpholin-1-yl)=CH₂.

43. The compound according to any one of claims 1-38, wherein R¹ is —NH—C(O)—C≡C-CH₂-NMe₂,-NH-C(O)-C(H)═C(H)-CH₂-NMe₂, —NH—C(O)—C(H)═C(H)-CH₂-(3,3-difluoroazetidin-1-yl), —NH—C(O)—C≡C-Me, —NH—C(O)—C(CH₂-SO₂Me)=CH₂, —NH—C(O)—C(CH₂-CH₂-NMe₂)=CH₂, —NH—C(O)—C(CH₂-(3,3-difluoroazetidin-1-yl))=CH₂, —NH—C(O)—C(CH₂-(3-F-3-Me-azetidin-1-yl))=CH₂, —NH—C(O)—C(CH₂-N(Me)(t-Bu))=CH₂, —NH—C(O)—C≡C-CH₂-(3,3-difluoroazetidin-1-yl), —NH—C(O)—C(CH₂-(3-F-azetidin-1-yl))=CH₂, —NH—C(O)—C(CH₂-(3-F-3-hydroxymethyl-azetidin-1-yl))=CH₂, or —NH—C(O)—C(H)═C(H)-CH₂-OMe.

44. The compound according to any one of claims 1-38, wherein R¹ is —C(O)—C≡C-CH₂-NMe₂,-C(O)-C(H)═C(H)-CH₂-NMe₂,-C(O)—C(H)-C(H)-CH₂-(3,3-difluoroazetidin-1-yl),-C(O)—C≡C-Me,-C(O)-C(CH₂-SO₂Me)-CH₂,-C(O)—C(CH₂-CH₂-NMe₂)=CH₂,-C(O)—C(CH₂-(3,3-difluoroazetidin-1-yl))=CH₂, —C(O)—C(CH₂-(3-F-3-Mc-azctidin-1-yl))=CH₂,-C(O)—C(CH₂-N(Mc)(t-Bu))=CH₂,-C(O)—C≡C-CH₂-(3,3-difluoroazetidin-1-yl),-C(O)—C(CH₂-(3-F-azetidin-1-yl))=CH₂,-C(O)—C(CH₂-(3-F-3-hydroxymethyl-azetidin-1-yl))-CH₂,-C(O)—C(H)═C(H)-CH₂-OMe, or -C(O)—C≡C-CH₂-OMe.

45. The compound according to any one of claims 1-38, wherein R¹ is -CH₂—C≡C—C(O)-NMe₂ or C(O)-C≡C-CH₂-OMe.

46. The compound according to any one of claims 1-38, wherein R¹ is

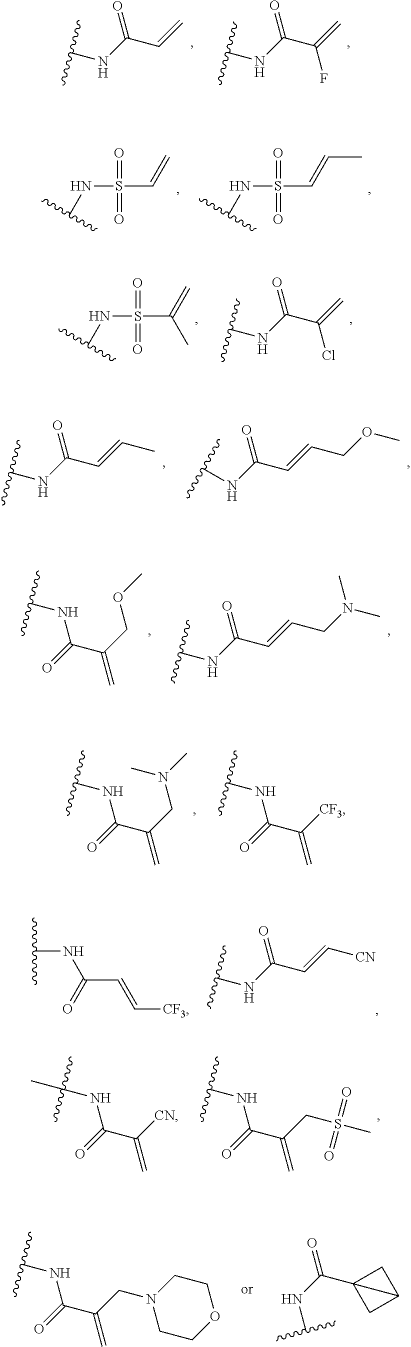

47. The compound according to any one of claims 1-38, wherein R¹ is

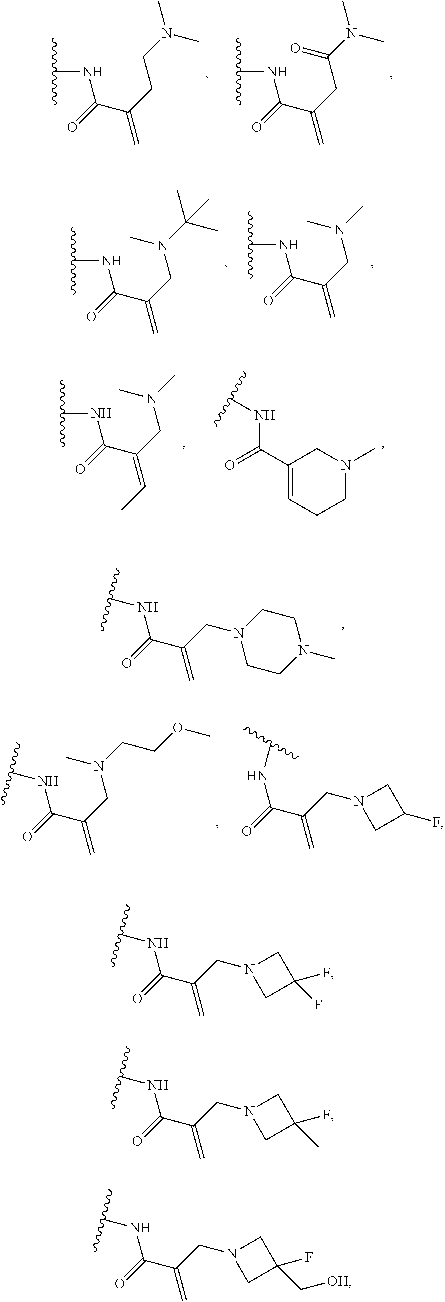

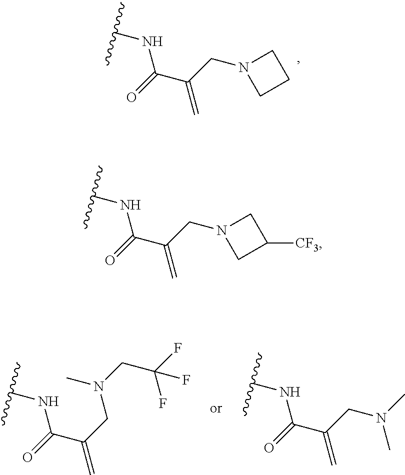

48. The compound according to any one of claims 1-38, wherein R¹ is

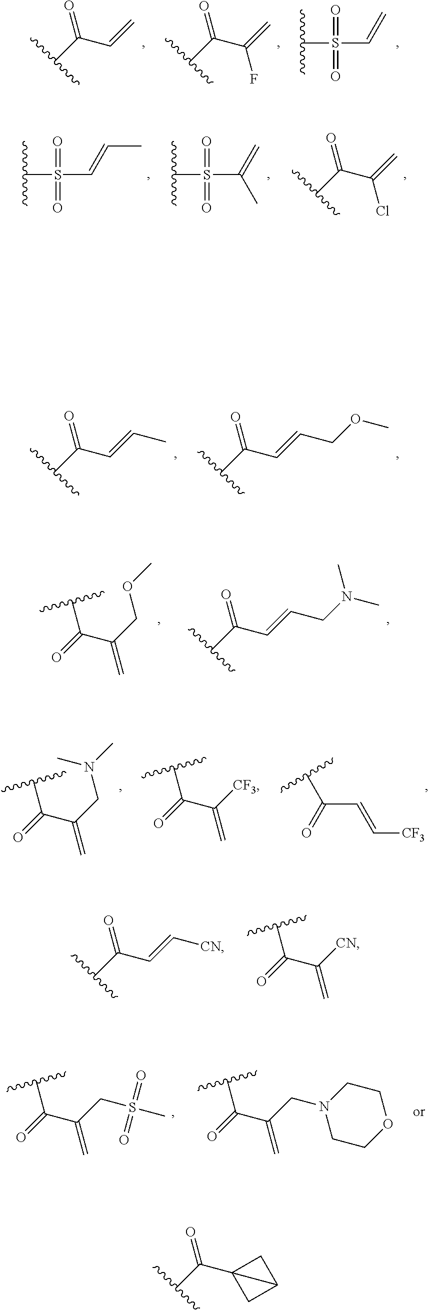

49. The compound according to any one of claims 1-38, wherein R¹ is

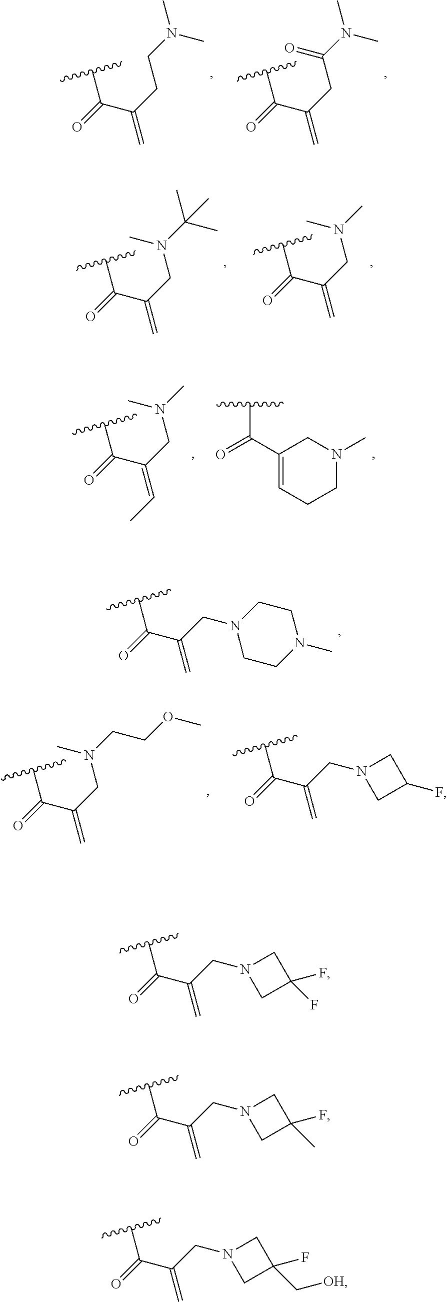

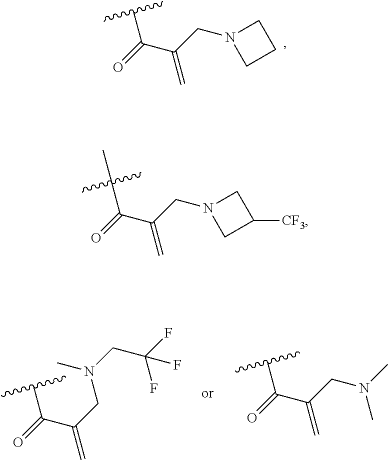

50. The compound according to any one of claims 1-38, wherein R¹ is -CH₂—C≡C—C(O)-NMe₂,-CH₂—C≡C-C(O)-NEt₂, or -CH₂-C(H)═C(H)-C(O)—(3,3-difluoroazetidin-1-yl).

51. The compound according to any one of claims 1-38, wherein R¹ is

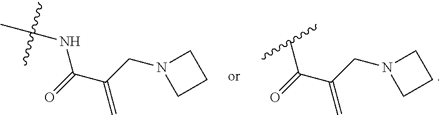

52. The compound according to any one of claims 1-38, wherein R¹ is —C(O)—C≡C-CH₂OMc, C(O)-C≡C-CH₂OH, C(O)-C≡C-C(Me)HOH.

53. The compound according to any one of claims 1-38, wherein R¹ is 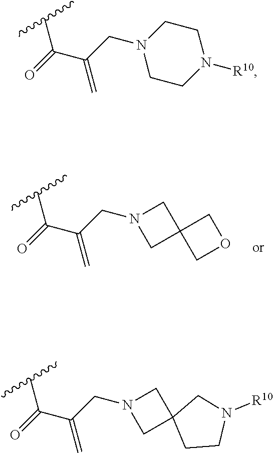
and R¹⁰ is —C(O)R¹¹ or —S(O)₂R¹¹; and R¹¹ is substituted or unsubstituted alkyl, substituted or unsubstituted aryl, or substituted or unsubstituted heteroaryl.

54. The compound according to claim 53, wherein R¹¹ is Me, Et, i-Pr, Ph, or pyridyl.

55. The compound according to claim 530, wherein R¹¹ is -CH₂—NH—C(O) Me,-C(Me) H—NH—C(O) Me, or -C(i-Pr) H—NH—C(O) Me.

56. The compound according to claim 1, wherein the substitution on each alkyl is 1, 2, or 3 groups independently selected from halo, CN, hydroxy, and alkoxy.

57. The compound according to claim 1, wherein the substitution on each alkoxy is 1, 2, or 3 groups independently selected from halo, CN, and substituted or unsubstituted alkyl.

58. The compound according to claim 1, wherein the substitution on each cycloalkyl is 1, 2, or 3 groups independently selected from halo, CN, hydroxy, substituted or unsubstituted alkyl, and substituted or unsubstituted alkoxy.

59. The compound according to claim 1, wherein the substitution on each heterocycloalkyl is 1, 2, or 3 groups independently selected from halo, CN, hydroxy, substituted or unsubstituted alkyl, and substituted or unsubstituted alkoxy.

60. The compound according to claim 1, wherein the substitution on each aryl is independently 1-3 groups independently selected from halo, haloalkyl, amino, dialkylamino, amido, CN, hydroxy, substituted or unsubstituted alkyl, and substituted or unsubstituted alkoxy.

61. The compound according to claim 1, wherein the substitution on each heteroaryl is independently 1-3 groups independently selected from halo, haloalkyl, amino, dialkylamino, amido, CN, hydroxy, substituted or unsubstituted alkyl, substituted or unsubstituted alkoxy, and oxo.

62. The compound according to claim 1, wherein the compound is any one of compounds listed in Table 1G.

63. The compound according to claim 1, wherein the compound is any one of compounds listed in Table 1H.

64. The compound according to claim 1, wherein the compound is any one of compounds listed in Table 1J.

65. The compound according to claim 1, wherein the compound is any one of compounds listed in Table IK.

66. The compound according to claim 1, wherein the compound is a compound listed in Table 1K; and wherein the compound ID is 915, 1033, 1036, 1087, 1092, 1094, 1095, 1097, 1098, or 1102.

67. A pharmaceutical composition comprising a therapeutically effective amount of a compound of any one of claims 1-66; or a pharmaceutically acceptable salt, solvate, or prodrug thereof; and a pharmaceutically acceptable excipient.

68. The pharmaceutical composition of claim 67 that is formulated for a route of administration selected from oral administration, parenteral administration, buccal administration, nasal administration, topical administration, or rectal administration.

69. A method for treating an autoimmune disease or condition comprising administering to a patient in need thereof a therapeutically effective amount of the pharmaceutical composition of claim 67 or 68.

70. A method for treating a heteroimmune disease or condition comprising administering to a patient in need thereof the pharmaceutical composition of claim 67 or 68.

71. A method for treating a cancer comprising administering to a patient in need thereof a therapeutically effective amount of the pharmaceutical composition of claim 67 or 68.

72. The method of claim 52, wherein the cancer is a B-cell proliferative disorder.

73. The method of claim 52, wherein the B-cell proliferative disorder is diffuse large B cell lymphoma, follicular lymphoma, chronic lymphocytic leukemia, lymphoid leukemia, ALL, soft tissue tumor, Glioblastoma, pancreatic tumor, or renal cell cancer.

74. The use of a compound or a metabolite, a solvate, a pharmaceutically acceptable salt, or a prodrug thereof, according to any one of claims 1-66, or a pharmaceutical composition of either of claim 67 or 68, in the manufacture of a medicament.

75. A compound or a metabolite, a solvate, a pharmaceutically acceptable salt, or a prodrug thereof, according to any one of claims 1-66, or a pharmaceutical composition of either of claim 67 or 68, for use as a medicament.

76. A compound or a metabolite, a solvate, a pharmaceutically acceptable salt, or a prodrug thereof, according to any one of claims 1-66, or a pharmaceutical composition of either of claim 67 or 68, for use in the treatment, prevention, or prophylaxis of autoimmune diseases, heteroimmune diseases, proliferative diseases, and inflammatory conditions.

77. A compound or a metabolite, a solvate, a pharmaceutically acceptable salt, or a prodrug thereof, according to any one of claims 1-66, or a pharmaceutical composition of either of claim 67 or 68, for use in the treatment, prevention, or prophylaxis of cancer, mastocytosis, B-cell lymphoma, lupus, and osteoporosis/bone resorption.

78. The use of a compound or a metabolite, a solvate, a pharmaceutically acceptable salt, or a prodrug thereof, according to any one of claims 1-66 in the preparation of a medicament for the treatment, prevention, or prophylaxis of an autoimmune disease, heteroimmune disease, proliferative disease, or inflammatory condition.

79. The use of a compound or a metabolite, a solvate, a pharmaceutically acceptable salt, or a prodrug thereof, according to any one of claims 1-66 in the preparation of a medicament for the treatment, prevention, or prophylaxis of cancer, mastocytosis, B-cell lymphoma, lupus, and osteoporosis/bone resorption.

80. The compound according to any one of claims 1-66 and the method according to any one of claims 69-73, or the use according to any one of claims 74-79, wherein the compound is an inhibitor of menin.

81. A method of treating diabetes mellitus in a mammal comprising administering to the mammal a pharmaceutical formulation according to any one of claims 66-67.

82. The method of claim 81, wherein the diabetes mellitus is type 1 diabetes mellitus.

83. The method of claim 81, wherein the diabetes mellitus is type 2 diabetes mellitus.
